(19) United States
(12) Patent Application Publication
(10) Pub. No.: US 2026/0297089 A1
(43) Pub. Date: Oct. 1, 2026

(54) COMPOUNDS AND METHODS OF USE

(71) Applicant: Tango Therapeutics, Inc., Boston, MA (US)

(72) Inventor: Kevin M. Cottrell, Arlington, MA (US)

(21) Appl. No.: 19/666,017

(22) Filed: May 1, 2026

Related U.S. Application Data
(63) Continuation of application No. 18/833,315, filed on Jan. 26, 2023.
(60) Provisional application No. 63/303,409, filed on Jan. 26, 2022.
(60) Provisional application No. 63/435,210, filed on Dec. 23, 2022.

(51) Int. Cl. C07D 471/04; A61K 31/437; A61K 31/444; A61K 31/4725; A61K 31/506; A61K 45/06; C07D 213/75; C07D 213/82; C07D 401/14

(52) U.S. Cl. CPC C07D 471/04; A61K 31/437; A61K 31/444; A61K 31/4725; A61K 31/506; A61K 45/06; C07D 213/75; C07D 213/82; C07D 401/14

(57) ABSTRACT

Provided are compounds of Formula (A):

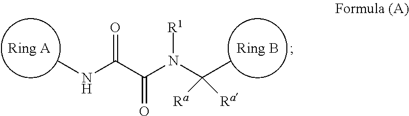

and pharmaceutically acceptable salts thereof, and pharmaceutical compositions, processes of preparing and methods of treating thereof; wherein R^{a}, R^{a}′, Ring A, Ring B, and R¹ are as defined in any of the embodiments described herein.

### CROSS-REFERENCE TO RELATED APPLICATIONS

This application is a continuation of U.S. application Ser. No. 18/833,315, filed on Jul. 25, 2024, which is a national phase filing under 35 U.S.C. § 371 of International (PCT) Patent Application No. PCT/US2023/011654, filed on Jan. 26, 2023, which claims priority to U.S. Provisional Application No. 63/303,409, filed on Jan. 26, 2022, and to U.S. Provisional Application No. 63/435,210, filed on Dec. 23, 2022, which are incorporated by reference herein in their entireties and for all purposes.

### FIELD

Provided herein are compounds, and compositions and methods thereof. In some embodiments, provided are compounds for inhibiting protein arginine methyltransferase 5 (PRMT5). In some embodiments, provided are methods for treatment of diseases or disorders, such as cancer.

### BACKGROUND

Protein arginine methyltransferase 5 (PRMT5) is a type II arginine methyltransferase that regulates essential cellular functions, including the regulation of cell cycle progression, apoptosis and the DNA-damage response (Koh, C. et al., Curr Mol Bio Rep 2015; Wu et al., Nat Rev Drug Discovery 2021). MTAP is a critical enzyme in the methionine salvage pathway, a six-step process that recycles methionine from the product of polyamine synthesis, methylthioadenosine (MTA). Loss of MTAP causes the accumulation of its substrate, MTA, which has been described to function as a SAM-competitive PRMT5 inhibitor (Kruykov et al., 2016; Marjon et al., 2016 and Markarov et al., 2016). Data from genome-wide genetic perturbation screens using shRNA suggests a selective requirement for PRMT5 activity particularly in MTAP-deleted cancer cell lines (Kruykov et al., 2016; Marjon et al., 2016 and Markarov et al., 2016). It is proposed that the accumulation of MTA caused by MTAP-deletion in these cell lines partially inhibits PRMT5, rendering those cells selectively sensitive to additional PRMT5 inhibition.

A PRMT5 inhibitor that leverages the accumulation of MTA by binding in an MTA-uncompetitive, non-competitive or mixed mode manner or in a MTA-cooperative binding manner may demonstrate selectivity for MTAP-deleted tumor cells. Some PRMT5 inhibitors are currently being explored for therapeutic uses (e.g., for treating cancer), however there are currently no such PRMT5 therapies approved by the United States Food and Drug Administration that demonstrate selectivity for MTAP-deleted cancer cell lines.

Accordingly, there is a need for PRMT5 inhibitors for treating diseases, such as cancers.

### SUMMARY

In one embodiment, provided herein are compounds of Formula (A) or pharmaceutically acceptable salts thereof:
wherein:

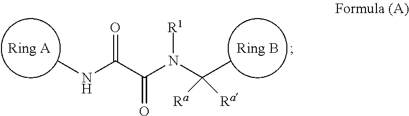

Ring A is selected from the group consisting of:

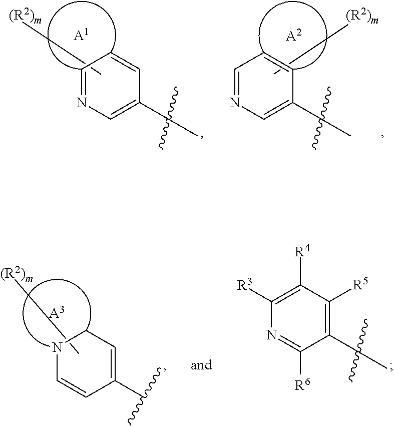

Ring B is selected from the group consisting of C₆-C₁₀ aryl and 5-10 membered heteroaryl, each optionally substituted at any available position;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₁-C₆ alkyl, —C₂-C₆ heteroalkyl, —C₂-C₆ haloalkyl, —C₃-C₁₀ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position;
each R² is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(—O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a} and R^{a}′ are independently selected from H and C₁-C₆ alkyl;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, C(═O)R^{b}, —C(═O)OR^{b}, —NR^{b}C(═O)R′, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu) and C₃-C₉ cycloalkyl (e.g., cyclopropyl, cyclobutyl, cyclopentyl, cyclohexyl); and
m is 0, 1, 2 or 3; wherein
(i) when R⁴ is —CH₃ then R³ is not H and;
(ii) the compound is not one of compounds a) to k) or a pharmaceutically acceptable salt thereof:
a)N1-benzyl-N2-(8-fluoroquinolin-3-yl)-N1-(2-(2-methoxyethoxy)ethyl)oxalamide:

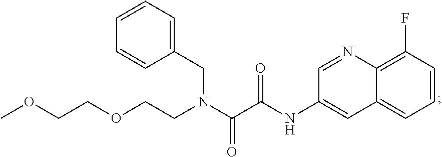 [SMILES: COCCOCCN(Cc1ccccc1)C(=O)C(=O)Nc1cnc2c(F)cccc2c1]

b)N1-(8-fluoroquinolin-3-yl)-N2-phenethyl-N2-(pyridin-4-ylmethyl)oxalamide:

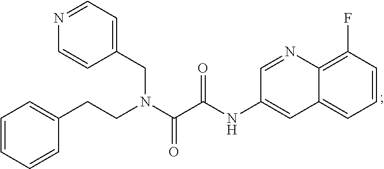 [SMILES: O=C(Nc1cnc2c(F)cccc2c1)C(=O)N(CCc1ccccc1)Cc1ccncc1]

c)N1-(6-amino-5,6,7,8-tetrahydroquinolin-3-yl)-N2-(3-fluoro-4-(pyridin-3-yl)benzyl)-N2-methyloxalamide:

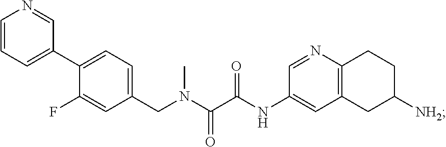 [SMILES: CN(Cc1ccc(-c2cccnc2)c(F)c1)C(=O)C(=O)Nc1cnc2c(c1)CC(N)CC2]

d)N1-cyclopentyl-N1-(3-fluorobenzyl)-N2-(8-fluoroquinolin-3-yl)oxalamide:

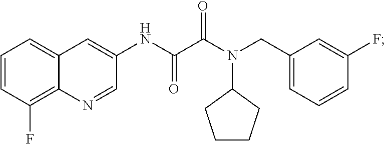 [SMILES: O=C(Nc1cnc2c(F)cccc2c1)C(=O)N(Cc1cccc(F)c1)C1CCCC1]

e)N1-(4-carbamoylbenzyl)-N2-(8-fluoroquinolin-3-yl)-N1-methyloxalamide:

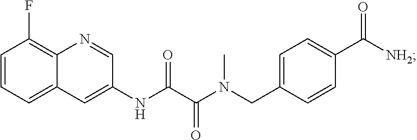 [SMILES: CN(Cc1ccc(C(N)=O)cc1)C(=O)C(=O)Nc1cnc2c(F)cccc2c1]

f)N1-benzyl-N1-methyl-N2-(quinolin-3-yl)oxalamide:

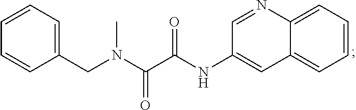 [SMILES: CN(Cc1ccccc1)C(=O)C(=O)Nc1cnc2ccccc2c1]

g)N1-([1,2,4]triazolo[4,3-a]pyridin-3-ylmethyl)-N2-(5-((dimethylamino)methyl)pyridin-3-yl)-N1-methyloxalamide:

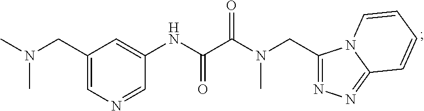 [SMILES: CN(C)Cc1cncc(NC(=O)C(=O)N(C)Cc2nnc3ccccn23)c1 ; ";"]

h)N1-(4-bromo-2-(3-chlorophenoxy)benzyl)-N2-(5-bromo-2-(4-methylpiperazin-1-yl)pyridin-3-yl)-N1-methyloxalamide:

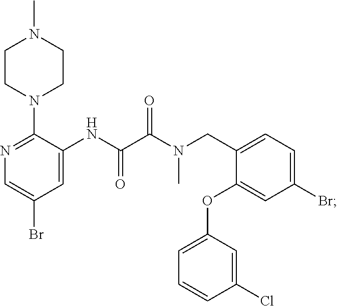 [SMILES: CN1CCN(c2ncc(Br)cc2NC(=O)C(=O)N(C)Cc2ccc(Br)cc2Oc2cccc(Cl)c2)CC1]

i) methyl 4-((N-methyl-2-oxo-2-((5-(trifluoromethyl)pyridin-3-yl)amino)acetamido)methyl)benzoate:

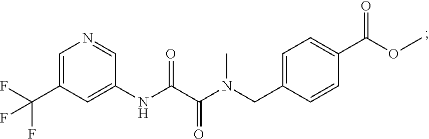 [SMILES: COC(=O)c1ccc(CN(C)C(=O)C(=O)Nc2cncc(C(F)(F)F)c2)cc1]

j)N1-methyl-N1-(2-methylbenzyl)-N2-(2-oxo-5-(trifluoromethyl)-1,2-dihydropyridin-3-yl)oxalamide:

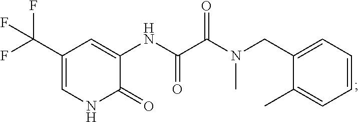 [SMILES: Cc1ccccc1CN(C)C(=O)C(=O)Nc1cc(C(F)(F)F)c[nH]c1=O ; ";"]

k)N1-(furan-2-ylmethyl)-N1-methyl-N2-(2-oxo-5-(trifluoromethyl)-1,2-dihydropyridin-3-yl)oxalamide:

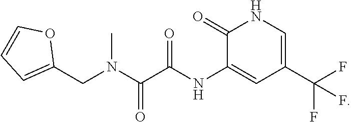 [SMILES: CN(Cc1ccco1)C(=O)C(=O)Nc1cc(C(F)(F)F)c[nH]c1=O]

In one embodiment, provided is a pharmaceutical composition comprising a compound of Formula (A), or a pharmaceutically acceptable salt thereof, as defined in any of the embodiments described herein and a pharmaceutically acceptable carrier. In some embodiments, the pharmaceutical composition further comprises a second therapeutic agent.

In one embodiment, provided is a method of treating an MTAP-deficient and/or an MTA-accumulating disease in a subject in need thereof by administering to the subject an effective amount (e.g., a therapeutically effective amount) of compound of Formula (A), or a pharmaceutically acceptable salt thereof, as defined in any of the embodiments described herein or a pharmaceutically acceptable composition thereof. In some embodiments, the compound or composition is administered in combination with a second therapeutic agent.

In one embodiment, provided is a method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering an effective amount (e.g., a therapeutically effective amount) of a compound of Formula (A) as defined in any of the embodiments described herein or a pharmaceutical composition thereof to the subject identified in step b).

In an embodiment, provided is a use of a compound of Formula (A), or a pharmaceutically acceptable salt thereof, as defined in any of the embodiments described herein, or of a pharmaceutically acceptable composition as described herein for treating an MTAP-deficient and/or an MTA-accumulating disease in a subject in need thereof. In an embodiment, the compound or composition is configured to be administered in combination with a second therapeutic agent.

In an embodiment, provided is a compound of Formula (A), or a pharmaceutically acceptable salt thereof, as defined in any of the embodiments described herein, or a pharmaceutically acceptable composition as described herein for treating an MTAP-deficient and/or an MTA-accumulating disease in a subject in need thereof. In an embodiment, the compound or composition is configured to be administered in combination with a second therapeutic agent.

In an embodiment, provided is a use of a compound of compound of Formula (A), or a pharmaceutically acceptable salt thereof, as defined in any of the embodiments described herein, or of a pharmaceutically acceptable composition as described herein in the manufacturing of a medicament for treating an MTAP-deficient and/or an MTA-accumulating disease in a subject in need thereof. In an embodiment, the medicament is configured to be administered in combination with a second therapeutic agent.

### DETAILED DESCRIPTION

The disclosure herein sets forth exemplary methods, parameters and the like. It should be recognized, however, that such description is not intended as a limitation on the scope of the present disclosure but is instead provided as a description of exemplary embodiments.

As generally described herein, provided are compounds (e.g., compounds of Formula (A) or compounds of Table 1, or pharmaceutically acceptable salts thereof) that are MTA-uncompetitive PRMT5 inhibitors useful for treating proliferating disorders (e.g., cancers) associated with MTAP deficiencies and/or MTA accumulation.

In some embodiments, provided are compounds (e.g., compounds of Formula (A) or compounds of Table 1, or pharmaceutically acceptable salts thereof) that are MTA-uncompetitive, non-competitive or mixed mode PRMT5 inhibitor or an MTA cooperative binding agent useful for treating proliferating disorders (e.g., cancers) associated with MTAP deficiencies and/or MTA accumulation.

### Definitions

As used in the present disclosure, the following words and phrases are generally intended to have the meanings as set forth below unless expressly indicated otherwise or the context in which they are used indicates otherwise.

### MTAP

“MTAP” as used herein refers to methylthioadenosine phosphorylase, an enzyme in the methionine salvage pathway, also known as S-methyl-5′-thioadenosine phosphorylase; also known as BDMF; DMSFH; DMSMFH; LGMBF; MSAP; and c86fus. External IDs: OMIM: 156540 MGI: 1914152 HomoloGene: 1838 chEMBL: 4941 GeneCards: MTAP Gene; Entrez 4507; RefSeq (mRNA): NM_002451; location: Chr 9:21.8-21.93 Mb. By “wild-type” MTAP is meant that encoded by NM_002451 or having the same amino acid sequence (NP_002442). (Schmid et al. Oncogene 2000, 19, pp 5747-54).

As used herein, the term “MTAP-deficient”, “MTAP-deficiency”, “MTAP-null” and the like refer to cells (including, but not limited to, cancer cells, cell lines, tissues, tissue types, tumors, etc.) that have a significant reduction in post-translational modification, production, expression, level, stability and/or activity of MTAP relative to that in a control, e.g., reference or normal or non-cancerous cells. The reduction can be at least about 20%, 30%, 40%, 50%, 60%, 70%, 80% or 90%. In some embodiments, the reduction is at least 20%. In some embodiments, the reduction is at least 50%. The terms “MTAP-deficient and/or MTA accumulating”, “MTAP-deficient and/or MTA-accumulating”, MTAP deficient and/or MTA upregulated” and the like, regarding a cell or cells, etc., indicate that the cell or cells, etc., either are deficient in MTAP and/or overproduce or accumulate MTA. MTAP-deficient cells include those wherein the MTAP gene has been mutated, deleted, or transcriptionally silenced. As a non-limiting example, MTAP-deficient cells can have a homozygous deletion. MTAP knockdown is not lethal. In some embodiments, the MTAP-deficient cells are also CDKN2A-deficient. The MTAP deficiency can be detected using any reagent or technique known in the art, for example: immunohistochemistry utilizing an antibody to MTAP, and/or genomic sequencing, and/or nucleic acid hybridization and/or amplification utilizing at least one probe or primer comprising a sequence of at least 12 contiguous nucleotides (nt) of the sequence of MTAP, wherein the primer is no longer than about 30 nt.

An “MTAP-deficiency-related” or “MTAP-deficiency” or “MTAP deficient” disease (for example, a proliferating disease, e.g., a cancer) or a disease (for example, a proliferating disease, e.g., a cancer) “associated with MTAP deficiency” or a disease (for example, a proliferating disease, e.g., a cancer) “characterized by MTAP deficiency” and the like refer to an ailment (for example, a proliferating disease, e.g., a cancer) wherein a significant number of cells are MTAP-deficient. For example, in a MTAP-deficiency-related disease, one or more disease cells can have a significantly reduced post-translational modification, production, expression, level, stability and/or activity of MTAP. Examples of MTAP-deficiency-related diseases include, but are not limited to, cancers, including but not limited to: glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma (See FIG. 1). In a patient afflicted with a MTAP-deficiency-related disease, it is possible that some disease cells (e.g., cancer cells) can be MTAP-deficient while others are not. Similarly, some disease cells may be MTA-accumulating while others are not.

Thus, the present disclosure encompasses methods of treatment involving diseases of these tissues, or any other tissues, wherein the proliferation of MTAP-deficient and/or MTA-accumulating cells can be inhibited by administration of a PRMT5 inhibitor. Some cancer cells which are MTAP-deficient are also deficient in CDKN2A; the post-translational modification, production, expression, level, stability and/or activity of the CDKN2A gene or its product are decreased in these cells. The genes for MTAP and CDKN2A are in close proximity on chromosome 9p21; MTAP is located approximately 100 kb telomeric to CDKN2A. Many cancer cell types harbor CDKN2A/MTAP loss (loss of both genes). Thus, in some embodiments, a MTAP-deficient cell is also deficient in CDKN2A.

### MTA and MTA Accumulation

By “MTA” is meant the PRMT5 inhibitor also known as methyl-thioadenosine, S-methyl-5′-thioadenosine, [5′deoxy-5′-(methylthio)-fl-D-ribofuranosyl]adenine, 5′-methyl-thioadenosine, 5′-deoxy, 5′-methyl thioadenosine, and the like. MTA selectively inhibits PRMT5 methyltransferase activity. MTA is the sole known catabolic substrate for MTAP. The terms “MTA accumulating”, “MTA overproducing”, “MTA upregulated” and the like refer to cells (including, but not limited to, cancer cells, cell lines, tissues, tissue types, tumors, etc.) that have a significantly increased production, level and/or stability of MTA. MTA-accumulating cells include those wherein the cells comprise at least about 20%, 30%, 40%, 50%, 60%, 70%, 80%, 90%, 100%, or greater than 100%, higher production, level and/or stability of MTA than that in normal or non-cancerous cells. In some embodiments, MTA-accumulating cells include those wherein the cells comprise at least 20% higher production, level and/or stability of MTA than that in normal or non-cancerous cells. In some embodiments, MTA-accumulating cells include those wherein the cells comprise at least 50% higher production, level and/or stability of MTA than that in normal or non-cancerous cells. Determination of MTA accumulation in test samples (e.g., cells such as cancer cells being tested for MTA accumulation) and reference samples, and other cells, tissues, samples, etc., can be performed using any method known in the art. Such methods for detecting MTA include, as a non-limiting example, liquid chromatography-electrospray ionization-tandem mass spectrometry (LC-ESI-MS/MS), as described in Stevens et al. J. Chromatogr. A. 2010, 1217, pp 3282-3288; and Kirovski et al. Am. J. Pathol. 2011, 178, pp 1145-1152; and references cited therein. Loss of MTAP is associated with accumulation of MTA (Williams-Ashman et al. Biochem. Pharm. 1982, 31, pp 277-288; and Limm et al. Eur. J. Cancer. 2013, 49, Issue 6.

An “MTA-accumulation-related”, “MTA-accumulation”, “MTA-accumulating”, “MTA overproducing”, “MTA upregulated” disease (for example, a proliferating disease, e.g., a cancer) or a disease (for example, a proliferating disease, e.g., a cancer) “associated with MTA accumulation” or a disease (for example, a proliferating disease, e.g., a cancer) “characterized by MTA accumulation” and the like refer to an ailment (for example, a proliferating disease, e.g., a cancer) wherein a significant number of cells are MTA accumulating. Examples of MTA-accumulating diseases include, but are not limited to, cancers, including but not limited to: glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma (See FIG. 1). In a patient afflicted with a MTAP-deficiency-related disease, it is possible that some disease cells (e.g., cancer cells) can be MTAP-deficient while others are not.

In a patient having or having been diagnosed with an MTA-accumulating disease, some cells may be MTA-accumulating while others are not.

An increase in therapeutic window between normal cells and MTAP-deleted/MTA accumulating cells could be achieved by using an inhibitor that binds PRMT5 uncompetitively with MTA. As used herein, “uncompetitive binding” and “uncompetitive inhibition” and “cooperative binding” and “cooperative inhibition” (e.g., MTA-uncompetitive binding, MTA-uncompetitive inhibition, MTA-cooperative binding, MTA-cooperative inhibition) refers to binding of an inhibitor to a protein (e.g., PRMT5) that is increased in the presence of a co-factor (e.g., MTA) over the binding of the same inhibitor in the absence of the co-factor. The PRMT5 inhibitors known in the art are generally either SAM (S-adenosylmethionine) uncompetitive or SAM competitive. As the concentration of SAM in wild-type and MTAP-null cells is similar, these inhibitors are expected to bind with similar potency to both cell types. By contrast, an MTA-cooperative (and either SAM competitive or showing enhanced cooperativity with MTA relative to SAM) inhibitor would bind with apparent greater potency in the presence of high concentrations of MTA and would therefore result in preferential inhibition of PRMT5 in MTA-accumulating cells relative to normal cells.

As described further herein, a cancer cell, a cancer type, or a subject with cancer, is “PRMT5 inhibitor sensitive,” sensitive to treatment with PRMT5 inhibitors,” sensitive to PRMT5 therapeutic inhibition,” or described in similar terms if it is amenable to treatment with a PRMT5 inhibitor, e.g., due to its MTAP deficiency and/or MTA accumulation character.

### PRMT5

“PRMT5” as used herein is the gene or protein Protein Arginine Methyltransferase 5, also known as HRMTIL5; IBP72; JBP1; SKB1; or SKB1Hs External IDs: OMIM: 604045, MGI: 1351645, HomoloGene: 4454, ChEMBL: 1795116, GeneCards: PRMT5 Gene; EC number 2.1.1.125. Ensembl ENSG00000100462; UniProt 014744; Entrez Gene ID: 10419; RefSeq (mRNA): NM_001039619. The mouse homolog is NM_013768. Methyltransferases such as PRMT5 catalyze the transfer of one to three methyl groups from the co-factor S-adenosylmethionine (also known as SAM or AdoMet) to lysine or arginine residues of histone proteins. Arginine methylation is carried out by 9 different protein arginine methyltransferases (PRMT) in humans. Three types of methylarginine species exist: (1) Monomethylarginine (MMA); (2) Asymmetric dimethyl arginine (ADMA), which is produced by Type I methyl transferases (PRMT1, PRMT2, PRMT3, CARMI, PRMT6 and PRMT8); and (3) Symmetrical dimethylarginine (SDMA), which is produced by Type II methyl transferases (PRMT5 and PRMT7). PRMT1 and PRMT5 are the major asymmetric and symmetric arginine methyltransferases, respectively. PRMT5 promotes symmetric dimethylation on histones at H3R8 and H4R³ (H4R3me2). Symmetric methylation of H4R3 is associated with transcriptional repression and can act as a binding site for DNMT3A. Loss of PRMT5 results in reduced DNMT3A binding and gene activation. Tumor suppressor gene ST7 and chemokines RNATES, IP10, CXCL11 are targeted and silenced by PRMT5. WO 2011/079236.

Additional substrates include E2F1, p53, EGFR and CRAF. PRMT5 is part of a multi-protein complex comprising the co-regulatory factor WDR77 (also known as MEP50, a CDK4 substrate) during G1/S transition. Phosphorylation increases PRMT5/WDR77 activity. WDR77 is the non-catalytic component of the complex and mediates interactions with binding partners and substrates. PRMT5 can also interact with pICIn or RioK1 adaptor proteins in a mutually exclusive fashion to modulate complex composition and substrate specificity.

PRMT5 has either a positive or negative effect on its substrates by arginine methylation when interacting with a number of complexes and is involved in a variety of cellular processes, including RNA processing, signal transduction, transcriptional regulation, and germ cell development. PRMT5 is a major pro-survival factor regulating eIF4E expression and p53 translation. PRMT5 triggers p53-dependent apoptosis and sensitized various cancer cells to Tumor necrosis factor (TNF)-related apoptosis-inducing ligand (TRAIL) without affecting TRAIL resistance in non-transformed cells.

The term “PRMT5 inhibitor” refers to any compound capable of inhibiting the production, level, activity, expression or presence of PRMT5. These include, as non-limiting examples, any compound inhibiting the transcription of the gene, the maturation of RNA, the translation of mRNA, the posttranslational modification of the protein, the enzymatic activity of the protein, the interaction of same with a substrate, etc. The term also refers to any agent that inhibits the cellular function of the PRMT5 protein, either by ATP-competitive inhibition of the active site, allosteric modulation of the protein structure, disruption of protein-protein interactions, or by inhibiting the transcription, translation, post-translational modification, or stability of PRMT5 protein.

In some embodiments, a PRMT5 inhibitor competes with another compound, protein or other molecule which interacts with PRMT5 and is necessary for PRMT5 function. As a non-limiting example, a PRMT5 inhibitor can compete with the co-factor S-adenosylmethionine (also known as SAM or AdoMet).

In some embodiments, the PRMT5 inhibitor is uncompetitive with MTA. In some embodiments, the PRMT5 inhibitor is uncompetitive with MTA and competitive with SAM. In some embodiments, the PRMT5 inhibitor is uncompetitive with MTA and uncompetitive with SAM but binds with a higher degree of potency for the MTA complex relative to the SAM complex.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). Additionally encompassed are compounds described herein as individual isomers substantially free of other isomers, and alternatively, as mixtures of various isomers.

The “enantiomeric excess” (“e.e.”) or “% enantiomeric excess” (“% e.e.”) of a composition as used herein refers to an excess of one enantiomer relative to the other enantiomer present in the composition. For example, a composition can contain 90% of one enantiomer, e.g., the S enantiomer, and 10% of the other enantiomer, i.e., the R enantiomer.

$$e . e . = ( 90 - 10 ) / 100 = 80 \% .$$

Thus, a composition containing 90% of one enantiomer and 10% of the other enantiomer is said to have an enantiomeric excess of 80%.

The “diastereomeric excess” (“d.e.”) or “% diastereomeric excess” (“% d.e.”) of a composition as used herein refers to an excess of one diastereomer relative to one or more different diastereomers present in the composition. For example, a composition can contain 90% of one diastereomer, and 10% of one or more different diastereomers.

$$d . e . = ( 90 - 10 ) / 100 = 80 \% .$$

Thus, a composition containing 90% of one diastereomers and 10% of one or more different diastereomers is said to have a diastereomeric excess of 80%.

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

In a formula, [symbol] is a single bond where the stereochemistry of the moieties immediately attached thereto is not specified.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

The term “azido” refers to the radical —N₃.

“Aliphatic” refers to an alkyl, alkenyl, alkynyl, or carbocyclyl group, as defined herein. “Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group (e.g., a 3-10 membered heterocyclyl containing 1, 2 or 3 heteroatoms selected from N, O, S and oxidized forms thereof). In some embodiments, a heterocyclylalkyl is a C₁₋₂ alkyl-heterocyclyl (e.g., —CH₂-heterocyclyl, —CH₂CH₂-heterocyclyl, —CH(CH₃)-heterocyclyl). In some embodiments, a heterocyclylalkyl is a —CH₂-heterocyclyl. Typical heterocyclylalkyl groups include, but are not limited to, tetrahydrofuranylmethyl, tetrahydropyranylmethyl, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Aralkyl” or “arylalkyl” is a subset of alkyl and aryl, as defined herein, and refers to an alkyl group substituted by an aryl group (e.g., a C₆-C₁₀ aryl group). In some embodiments, arylalkyl is a C₁₋₂ alkyl-aryl (e.g., —CH₂-aryl, —CH₂CH₂-aryl, —CH(CH₃)-aryl). In some embodiments, arylalkyl is a —CH₂-aryl (e.g., —CH₂-phenyl, —CH₂-naphthyl). “Alkyl” refers to a radical of a straight-chain or branched saturated hydrocarbon group having from 1 to 20 carbon atoms (“C₁₋₂₀ alkyl” or “C₁-C₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C1-6 alkyl groups include methyl(C₁), ethyl(C₂), n-propyl(C₃), isopropyl(C₃), n-butyl(C₄), tert-butyl(C₄), sec-butyl(C₄), iso-butyl(C₄), n-pentyl(C₅), 3-pentanyl(C₅), amyl(C₅), neopentyl(C₅), 3-methyl-2-butanyl(C₅), tertiary amyl(C₅), and n-hexyl(C₆). Additional examples of alkyl groups include n-heptyl(C₇), n-octyl(C₅) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me(—CH₃), Et(—CH₂CH₃), ⁱPr(—CH(CH₃)₂), ⁿPr(—CH₂CH₂CH₃), ⁿBu(—CH₂CH₂CH₂CH₃), or ⁱBu(—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl(methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C (CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C (CH₃)₂CH₂—, —CH₂CH₂C (CH₃)₂—), and the like. When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds)(“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl(C₂), 1-propenyl(C₃), 2-propenyl(C₃), 1-butenyl(C₄), 2-butenyl(C₄), butadienyl(C₄), and the like. Examples of C₂₋₆ alkenyl groups include the aforementioned C₂₋₄ alkenyl groups as well as pentenyl(C₅), pentadienyl(C₅), hexenyl(C₆), and the like. Additional examples of alkenyl include heptenyl(C₇), octenyl(C₈), octatrienyl(C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds)(“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl(C₂), 1-propynyl(C₃), 2-propynyl(C₃), 1-butynyl(C₄), 2-butynyl(C₄), and the like. Examples of C2-6 alkenyl groups include the aforementioned C₂₋₄ alkynyl groups as well as pentynyl(C₅), hexynyl(C₆), and the like. Additional examples of alkynyl include heptynyl(C₇), octynyl(C₅), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl. Exemplary heteroalkyl groups include: —CH₂OH, —CH₂OCH₃, —CH₂NH₂, —CH₂NH(CH₃), —CH₂N(CH₃)₂, —CH₂CH₂OH, —CH₂CH₂OCH₃, —CH₂CH₂NH₂, —CH₂CH₂NH(CH₃), —CH₂CH₂N(CH₃)₂.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group is substituted with one or more of groups selected from halo, C₁-C₈alkyl, C₁-C₈ haloalkyl, cyano, hydroxy, C₁-C₈ alkoxy, and amino.

Examples of representative substituted aryls include the following

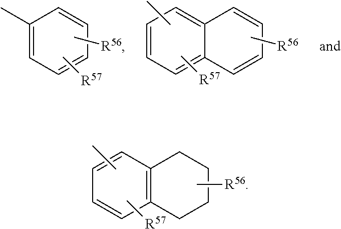

wherein one of R⁵⁶ and R⁵⁷ may be hydrogen and at least one of R⁵⁶ and R⁵⁷ is each independently selected from C₁-C₈alkyl, C₁-C₈ haloalkyl, 4-10 membered heterocyclyl, alkanoyl, C₁-C₈alkoxy, heteroaryloxy, alkylamino, arylamino, heteroarylamino, NR⁵⁸COR⁵⁹, NR⁵⁸SOR⁵⁹ NR⁵⁸SO₂R⁵⁹, COOalkyl, COOaryl, CONR⁵⁸R⁵⁹, CONR⁵⁸OR⁵⁹, NR⁵⁸R⁵⁹ SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group consisting of N, O, or S. R⁶⁰ and R⁶¹ are independently hydrogen, C₁-C₈alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbons in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, In such instances, unless otherwise specified, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In some embodiments, a heteroaryl group is a bicyclic 8-12 membered aromatic ring system having ring carbon atoms and 1-6 ring heteroatoms provided in the aromatic ring system, wherein each heteroatom is independently selected from nitrogen, oxygen, and sulfur (“8-12 membered bicyclic heteroaryl”). In some embodiments, a heteroaryl group is an 8-10 membered bicyclic aromatic ring system having ring carbon atoms and 1-6 ring heteroatoms provided in the aromatic ring system, wherein each heteroatom is independently selected from nitrogen, oxygen, and sulfur (“8-10 membered bicyclic heteroaryl”). In some embodiments, a heteroaryl group is a 9-10 membered bicyclic aromatic ring system having ring carbon atoms and 1-6 ring heteroatoms provided in the aromatic ring system, wherein each heteroatom is independently selected from nitrogen, oxygen, and sulfur (“9-10 membered bicyclic heteroaryl”). Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

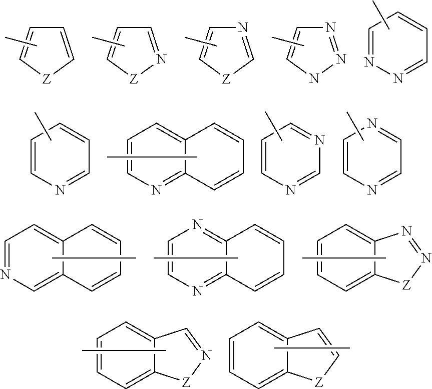

wherein each Z is selected from carbonyl, N, NR⁶⁵, O, and S; and R⁶⁵ is independently hydrogen, C₁-C₈alkyl, C₃-C₁₀ cycloalkyl, 4-10 membered heterocyclyl, C₆-C₁₀ aryl, and 5-10 membered heteroaryl.

In the structures described herein, a substituent attached to a polycyclic (e.g., bicyclic or tricyclic)cycloalkyl, heterocyclyl, aryl or heteroaryl with a bond that spans two or more rings is understood to mean that the substituent can be attached at any position in each of the rings.

“Heteroaralkyl” or “heteroarylalkyl” is a subset of “alkyl” and refers to an alkyl group substituted by a heteroaryl group (e.g., a 5-10 membered heteroaryl containing 1, 2 or 3 heteroatoms selected from O, N and S and oxidized forms thereof), wherein the point of attachment is on the alkyl moiety. In some embodiments, a heteroarylalkyl is a C₁₋₂ alkyl-heteroaryl (e.g., —CH₂-heteroaryl, —CH₂CH₂-heteroaryl, —CH(CH₃)-heteroaryl). In some embodiments, a heteroarylalkyl is a —CH₂-heteroaryl. Typical heteroarylalkyl groups include, but are not limited to, pyridinylmethyl, pyrimidinylmethyl, furanylmethyl, thiophenylmethyl, pyrolylmethyl, pyrazolylmethyl, imidazolylmethyl, thiazolylmethyl, oxazolylmethyl, thiazolylmethyl, pyridinylethyl, pyrimidinylethyl, furanylethyl, thiophenylethyl, pyrolylethyl, pyrazolylethyl, imidazolylethyl, thiazolylethyl, oxazolylethyl, thiazolylethyl and the like.

The term “carbocyclyl” or “carbocyclic” refers to a radical of a non-aromatic monocyclic, bicyclic, or tricyclic or polycyclic hydrocarbon ring system having from 3 to 14 ring carbon atoms (“C₃₋₁₄ carbocyclyl”) and zero heteroatoms in the non-aromatic ring system. Carbocyclyl groups include fully saturated ring systems (e.g., cycloalkyls), and partially saturated ring systems. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), and the like. Exemplary C₃₋₈ carbocyclyl groups include, without limitation, the aforementioned C₃₋₆ carbocyclyl groups as well as cycloheptyl(C₇), cycloheptenyl(C₇), cycloheptadienyl(C₇), cycloheptatrienyl(C₇), cyclooctyl(C₅), cyclooctenyl(C₈), bicyclo[2.2.1]heptanyl(C₇), bicyclo[2.2.2]octanyl(C₈), and the like. Exemplary C₃₋₁₀ carbocyclyl groups include, without limitation, the aforementioned C₃₋₈ carbocyclyl groups as well as cyclononyl(C₉), cyclononenyl(C₉), cyclodecyl(C₁₀), cyclodecenyl(C₁₀), octahydro-1H-indenyl(C₉), decahydronaphthalenyl(C₁₀), spiro[4.5]decanyl(C₁₀), and the like.

As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “cycloalkyl” as employed herein includes saturated cyclic, bicyclic, tricyclic, or polycyclic hydrocarbon groups having 3 to 14 carbons containing the indicated number of rings and carbon atoms (for example a C₃-C₁₄ monocyclic, C₄-C₁₄ bicyclic, C₅-C₁₄ tricyclic, or C₆-C₁₄ polycyclic cycloalkyl). In some embodiments “cycloalkyl” is a monocyclic cycloalkyl. In some embodiments, a monocyclic cycloalkyl has 3-14 ring carbon atoms. (“C₃₋₁₄ monocyclic cycloalkyl”). In some embodiments, a monocyclic cycloalkyl group has 3 to 10 ring carbon atoms (“C₃₋₁₀ monocyclic cycloalkyl”). In some embodiments, a monocyclic cycloalkyl group has 3 to 8 ring carbon atoms (“C₃₋₈ monocyclic cycloalkyl”). In some embodiments, a monocyclic cycloalkyl group has 3 to 6 ring carbon atoms (“C₃₋₆ monocyclic cycloalkyl”). In some embodiments, a monocyclic cycloalkyl group has 4 to 6 ring carbon atoms (“C₄₋₆ monocyclic cycloalkyl”). In some embodiments, a monocyclic cycloalkyl group has 5 to 6 ring carbon atoms (“C₅₋₆ monocyclic cycloalkyl”). In some embodiments, a monocyclic cycloalkyl group has 5 to 10 ring carbon atoms (“C₅₋₁₀ monocyclic cycloalkyl”). Examples of monocyclic C₅₋₆ cycloalkyl groups include cyclopentyl(C₅) and cyclohexyl(C₅). Examples of C₃₋₆ cycloalkyl groups include the aforementioned C₅₋₆ cycloalkyl groups as well as cyclopropyl(C₃) and cyclobutyl(C₄). Examples of C₃₋₈ cycloalkyl groups include the aforementioned C₃₋₆ cycloalkyl groups as well as cycloheptyl(C₇) and cyclooctyl(C₈).

In some embodiments “cycloalkyl” is a bicyclic cycloalkyl. In some embodiments, a bicyclic cycloalkyl has 4-14 ring carbon atoms. (“C₄₋₁₄ bicyclic cycloalkyl”). In some embodiments, a bicyclic cycloalkyl group has 4 to 12 ring carbon atoms (“C₄₋₁₂ bicyclic cycloalkyl”). In some embodiments, a bicyclic cycloalkyl group has 4 to 10 ring carbon atoms (“C₄₋₁₀ bicyclic cycloalkyl”). In some embodiments, a bicyclic cycloalkyl group has 5 to 10 ring carbon atoms (“C₅₋₁₀ bicyclic cycloalkyl”). In some embodiments, a bicyclic cycloalkyl group has 6 to 10 ring carbon atoms (“C₆₋₁₀ bicyclic cycloalkyl”). In some embodiments, a bicyclic cycloalkyl group has 8 to 10 ring carbon atoms (“C₈₋₁₀ bicyclic cycloalkyl”). In some embodiments, a bicyclic cycloalkyl group has 7 to 9 ring carbon atoms (“C₇₋₉ bicyclic cycloalkyl”). Examples of bicyclic cycloalkyls include bicyclo[1.1.0]butane (C4), bicyclo[1.1.1]pentane (C₅), spiro[2.2]pentane (C₅), bicyclo[2.1.0]pentane (C₅), bicyclo[2.1.1]hexane (C₆), bicyclo[3.1.0]hexane (C₆), spiro[2.3]hexane (C₆), bicyclo[2.2.1]heptane (norbornane)(C₇), bicyclo[3.2.0]heptane (C₇), bicyclo[3.1.1]heptane (C₇), bicyclo[3.1.1]heptane (C₇), bicyclo[4.1.0]heptane (C₇), spiro[2.4]heptane (C₇), spiro[3.3]heptane (C₇), bicyclo[2.2.2]octane (C₅), bicyclo[4.1.1]octane (C₅) octahydropentalene (C₈), bicyclo[3.2.1]octane (C₉), bicyclo[4.2.0]octane (C8), spiro[2.5]octane (C₉), spiro[3.4]octane (C₉), bicyclo[3.3.1]nonane (C₉), octahydro-1H-indene (C₉), bicyclo[4.2.1]nonane (C₉), spiro[3.5]nonane (C₉), spiro[4.4]nonane (C₉), bicyclo[3.3.2]decane (C₁₀), bicyclo[4.3.1]decane (C₁₀), spiro[4.5]decane (C₁₀), bicyclo[3.3.3]undecane (C₁₁), decahydronaphthalene (C₁₀), bicyclo[4.3.2]undecane (C₁₁), spiro[5.5]undecane (C₁₁) and bicyclo[4.3.3]dodecane (C₁₂).

In some embodiments “cycloalkyl” is a tricyclic cycloalkyl. In some embodiments, a tricyclic cycloalkyl has 6-14 ring carbon atoms. (“C₆₋₁₄ tricyclic cycloalkyl”). In some embodiments, a tricyclic cycloalkyl group has 8 to 12 ring carbon atoms (“C₈₋₁₂ tricyclic cycloalkyl”). In some embodiments, a tricyclic cycloalkyl group has 10 to 12 ring carbon atoms (“C ₁₀₋₁₂ tricyclic cycloalkyl. Examples of tricyclic cycloalkyls include adamantine (C₁₂).

Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In some embodiments, the heterocyclyl is a 3- to 10-membered non-aromatic ring system having ring carbon atoms and 1, 2 or 3 heteroatoms independently selected from nitrogen, oxygen and sulfur, including oxidized forms thereof. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. “Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical-C(═O)R20, where R20 is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O) Ph), benzylcarbonyl (—C(═O)CH₂Ph), —C(═O)—C₁-C₈ alkyl, —C(═O)—(CH₂):(C₆-C₁₀ aryl), —C(═O)—(CH₂):(5-10 membered heteroaryl), —C(═O)—(CH₂):(C₃-C₁₀ cycloalkyl), and —C(═O)—(CH₂); (4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

The term aminoalkyl refers to a substituted alkyl group wherein one or more of the hydrogen atoms are independently replaced by an —NH₂ group.

The term hydroxyalkyl refers to a substituted alkyl group wherein one or more of the hydrogen atoms are independently replaced by an —OH group.

The terms “alkylamino” and “dialkylamino” refer to —NH(alkyl) and —N(alkyl)₂ radicals respectively. In some embodiments the alkylamino is a —NH(C₁-C₄ alkyl). In some embodiments the alkylamino is methylamino, ethylamino, propylamino, isopropylamino, n-butylamino, iso-butylamino, sec-butylamino or tert-butylamino. In some embodiments the dialkylamino is —N(C₁-C₆ alkyl)₂. In some embodiments the dialkylamino is a dimethylamino, a methylethylamino, a diethylamino, a methylpropylamino, a methylisopropylamino, a methylbutylamino, a methylisobutylamino or a methyltertbutylamino.

The term “aryloxy” refers to an —O-aryl radical. In some embodiments the aryloxy group is phenoxy.

The term “haloalkoxy” refers to alkoxy structures that are substituted with one or more halo groups or with combinations thereof. For example, the term “fluoroalkoxy” includes haloalkoxy groups, in which the halo is fluorine. In some embodiments haloalkoxy groups are difluoromethoxy and trifluoromethoxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂):(C₆-C₁₀ aryl), —O—(CH₂):(5-10 membered heteroaryl), —O—(CH₂):(C₃-C₁₀ cycloalkyl), and —O—(CH₂)ₜ (4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂N(CH₃)₂.

“Amino” refers to the radical —NH₂.

“Oxo group” refers to —C(═O)—.

“Substituted amino” refers to an amino group of the formula —N(R³⁸)₂ wherein R³⁸ is

hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈alkyl, C₃-C₈ alkenyl, C₃-C₈ alkynyl, C₆-C₁₀ aryl, 5-10 membered heteroaryl, 4-10 membered heterocyclyl, or C₃-C10 cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)(C₆-C₁₀ aryl), —(CH₂):(5-10 membered heteroaryl), —(CH₂)(C₃-C₁₀ cycloalkyl), or —(CH₂)(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂):(C₆-C₁₀ aryl), —NR³⁹—(CH₂):(5-10 membered heteroaryl), —NR³⁹—(CH₂):(C₃-C₁₀ cycloalkyl), and —NR³⁹—(CH₂)ₜ (4-10 membered heterocyclyl), wherein t is an integer from 0 to 4, for instance 1 or 2, each R³⁹ independently represents H or C₁-C₈alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

In certain embodiments, the substituent present on the nitrogen atom is a nitrogen protecting group (also referred to herein as an “amino protecting group”). Nitrogen protecting groups include, but are not limited to, —OH, OR^{aa}, —N(RC)₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{c})₂, —SO₂R^{cc}, —SO₂OR^{cc}, —SOR^{aa}, —C(═S)N(R^{c})₂, —C(═O) SR^{cc}, C(═S)SR^{cc}, C₁₋₁₀ alkyl (e.g., aralkyl, heteroaralkyl), —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.
each instance of R^{aa} is, independently, selected from —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{c})₂, —CN, —C(═O)R^{aa}, —C(═O)N(R^{c})₂, —CO₂R^{aa}, —SO₂R^{aa}, —C(═NR^{cc})OR^{aa}, —C(═NR^{cc})N(R^{c})₂, —SO₂N(R^{c})₂, —SO₂R^{cc}, —SO₂OR^{cc}, —SOR^{aa}, —C(═S)N(R^{cc})₂, C(═O) SR^{cc}, —C(═S) SR^{cc}, —P(═O)(R^{aa})₂, —P(═O)(OR^{cc})₂, —P(═O)(N(R^{cc})₂)₂, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion.
each instance of R^{cc} is, independently, selected from hydrogen, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two Rec groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(Re) 3, —OSi(R^{ee})₃, —C(═S)N(R^{ff})₂, C(═O) SR^{ee}, —C(═S) SR^{ee}, —SC(═S) SR^{ee}, —P(═O)(OR^{ee})₂, —P(═O)(R^{ee})₂, —OP(═O)(R^{ee})₂, —OP(═O)(OR^{ee})R₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R 88 groups, or two geminal Rad substituents can be joined to form ═O or ═S; wherein X is a counterion;
each instance of Ree is, independently, selected from —C₁₋₆ alkyl, —C₁₋₆ perhaloalkyl, —C₂₋₆ alkenyl, —C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, —C₁₋₆ perhaloalkyl, C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R88 groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂ (C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS (C₁₋₆ alkyl), —C(═O)(C₁₋₆ alkyl), CO₂H, —CO₂ (C₁₋₆ alkyl), —OC(═O)(C₁₋₆ alkyl),0.0 (C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂ (C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, C(═NH)NH(C₁₋₆ alkyl), —C(═NH)NH₂, —OC(═NH)N(C₁₋₆ alkyl)₂, —OC(NH)NH(C₁₋₆ alkyl), —OC (NH)NH₂, —NHC (NH)N(C₁₋₆ alkyl)₂, —NHC(═NH)NH₂, —NHSO₂ (C₁₋₆ alkyl), —SO₂N(C₁₋₆ alkyl)₂, SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, OSi(C₁₋₆ alkyl)₃—C(═S)N(C₁₋₆ alkyl)₂, —C(═S)NH(C₁₋₆ alkyl), —C(═S)NH₂, —C(═O)S(C₁₋₆ alkyl), —C(═S) SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

For example, nitrogen protecting groups such as amide groups (e.g., C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo) fluorenylmethyl carbamate, 9-(2,7-dibromo) fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl) methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O) SR^{aa}, —C(═O)R^{aa}, —CO₂R^{aa}, C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{aa})₂, —P(OR)₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl(p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri (p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O) SR^{aa}, —C(═O)R^{aa}, —CO₂R^{a4}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR)₂, —P(OR^{cc})₃⁺X⁻, —P(═O)(R^{aa})₂, —P(═O)(OR° C.)₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

The term “leaving group” is given its ordinary meaning in the art of synthetic organic chemistry and refers to an atom or a group capable of being displaced by a nucleophile. Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In certain embodiments, the leaving group is halogen, alkanesulfonyloxy, arenesulfonyloxy, diazonium, alkyl diazenes, aryl diazenes, alkyl triazenes, aryl triazenes, nitro, alkyl nitrate, aryl nitrate, alkyl phosphate, aryl phosphate, alkyl carbonyl oxy, aryl carbonyl oxy, alkoxcarbonyl oxy, aryoxcarbonyl oxy ammonia, alkyl amines, aryl amines, hydroxyl group, alkyloxy group, or aryloxy. In some cases, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂ (CF₂)₃CF₃ (nonaflate, -ONf), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

“Carboxy” refers to the radical-C(═O)OH.

“Cyano” refers to the radical-CN.

“Halo” or “halogen” refers to fluoro (F), chloro (CI), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl (—CF₃), difluoromethyl (—CHF₂), fluoromethyl (—CH₂F), chloromethyl (—CH₂Cl), dichloromethyl (—CHCl₂), tribromomethyl (—CH₂Br), and the like.

“Hydroxy” refers to the radical-OH.

“Nitro” refers to the radical —NO₂.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. Any and all such combinations are contemplated in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb}) 3+X-, —N(OR)R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —S(═O)(═NR^{bb})R^{aa}, —OS(═O)R^{aa}, —Si(R^{aa})₃, —OSi(R^{aa})₃—C(═S)N(R^{bb})₂, —C(═O) SR^{aa}, —C(═S) SR^{aa}, —SC(═S) SR^{aa}, —SC(═O) SR^{aa}, —OC(═O) SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{c})₃, —B (R^{aa})₂, —B (OR^{cc})₂, —BR^{aa} (OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C (═O)R^{aa}, ═NNR^{bb}C (═O)OR^{aa}, -NNR^{bb}s (═O)₂R^{aa}, ═NR^{bb}, or ═NOR^{cc}; each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{c})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{c})₂, —SO₂N(R^{c})₂, —SO₂R°, —SO₂OR^{cc}, —SOR^{aa}, —C(═S)N(R^{cc})₂, —C(═O) SR^{cc}, —C(═S) SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})RR^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O) SR^{ee}, —C(═S) SR^{ee}, —SC(═S) SR^{ee}, —P(═O)₂R^{ee}, —P(═O)(R^{ee})₂, —OP(═O)(R^{ee})₂, —OP(═O)(OR^{ee})R₂, C₁₋₆ alkyl, C₁-6 haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of Ree is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R⁸⁸ is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂ X, —NH₂ (C₁₋₆ alkyl)⁺X⁻, —NH₃ X, —N(OC₁₋₆ alkyl)(C₁₋₆ alkyl), —N(OH)(C₁₋₆ alkyl), —NH(OH), —SH, —SC₁₋₆ alkyl, —SS (C₁₋₆ alkyl), —C(═O)(C₁₋₆ alkyl), —CO₂H, —CO₂ (C₁₋₆ alkyl), —OC(═O)(C₁₋₆ alkyl), —OCO₂ (C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂ (C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC (NH)NH(C₁₋₆ alkyl), —OC (NH)NH₂, —NHC (NH)N(C₁₋₆ alkyl)₂, —NHC(═NH)NH₂, —NHSO₂ (C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OCO C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S) SC₁₋₆ alkyl, —SC(═S)SC₁₋₆ alkyl, —P(═O)₂ (C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X- is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄-2sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{c})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{c})₂, —SO₂N(R^{c})₂, —SO₂R°, —SO₂OR^{cc}, —SOR^{aa}, —C(═S)N(R^{cc})₂, —C(═O) SR^{cc}, —C(═S) SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above. These and other exemplary substituents are described in more detail in the Detailed Description, Examples, and Claims. The invention is not intended to be limited in any manner by the above exemplary listing of substituents.

### Other Definitions

As used herein, the term “salt” refers to any and all salts and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomologus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In one embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response e.g., to treat a disease or disorder described herein. As will be appreciated by those of ordinary skill in this art, the effective amount of a compound of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the therapeutic treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the therapeutic treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Compounds

Provided herein are compounds of Formula (A). Unless the context requires otherwise, reference throughout this specification to “a compound of Formula (A)” or “compounds of Formula (A)” refers to all embodiments of Formula (A), including, for example, compounds of Formula (A), Formula (I), Formula (I_1), Formula (I_la), Formula (I_1b), Formula (I_1c), Formula (I_2), Formula (I_2a), Formula (I_3), Formula (I_3a), Formula (I_4), Formula (I_4a), Formula (I_4b), Formula (I_5), Formula (I_5a), Formula (I_5b), Formula (I_5c), Formula (I_5d), Formula (II), Formula (II_1), Formula (II_2), Formula (III), Formula (III_1), Formula (IV), Formula (V), Formula (VI), Formula (A_1), Formula (A_la), Formula (A_1b), Formula (A_1c), Formula (A_2), Formula (A_2a), Formula (A_3), Formula (A_3a), Formula (A_4), Formula (A_4a), Formula (A_4b), Formula (A_5), Formula (A_5a), Formula (A_5b), Formula (A_5c), Formula (A_5d), Formula (A-II), Formula (A-II_1), Formula (A-II_2), Formula (A-III), Formula (A-III_1), Formula (A-IV), Formula (A-V), Formula (A-VI)(i.e., Formula (A)-Formula (A-VI)) as well as the compounds of Table 1.

In an embodiment, the invention provides compounds of Formula (A) and pharmaceutically acceptable salts thereof. In an embodiment, the invention provides compounds of Formula (A) as the free base. In an embodiment, the invention provides compounds of Formula (A) as pharmaceutically acceptable salts).

In an embodiment, provided herein are compounds of Formula (A) or pharmaceutically acceptable salt thereof;
wherein:

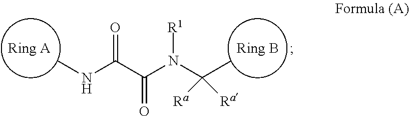

Ring A is selected from the group consisting of:

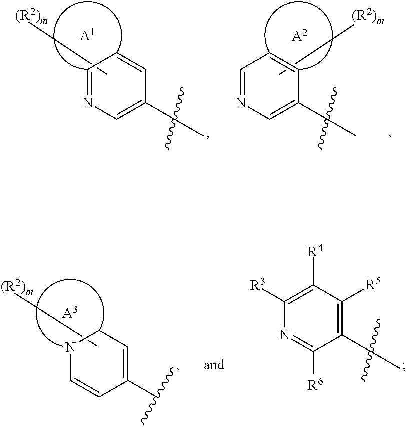

Ring B is selected from the group consisting of C₆-C₁₀ aryl and 5-10 membered heteroaryl, each optionally substituted at any available position;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₁-C₆ alkyl, —C₂-C₆ heteroalkyl, —C₂-C₆ haloalkyl, —C₃-C₁₀ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position;
each R² is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C6 alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a} and R^{a}′ are independently selected from H and C₁-C₆ alkyl;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, -—OR^{b}, —N(R^{b})₂, —C(═O)R^{b}, —C(═O)OR′, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R′, -NR'S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu) and C₃-C₉ cycloalkyl (e.g., cyclopropyl, cyclobutyl, cyclopentyl, cyclohexyl); and
m is 0, 1, 2 or 3; wherein
(i) when R⁴ is —CH₃ then R³ is not H and;
(ii) the compound is not one of compounds a) to k) or a pharmaceutically acceptable salt thereof:
a)N1-benzyl-N2-(8-fluoroquinolin-3-yl)-N1-(2-(2-methoxyethoxy)ethyl)oxalamide:

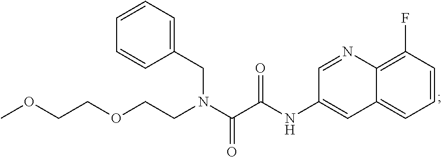 [SMILES: COCCOCCN(Cc1ccccc1)C(=O)C(=O)Nc1cnc2c(F)cccc2c1]

b)N1-(8-fluoroquinolin-3-yl)-N2-phenethyl-N2-(pyridin-4-ylmethyl)oxalamide:

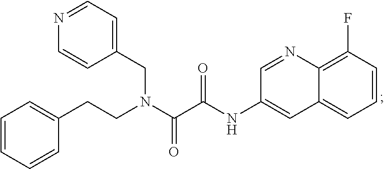 [SMILES: O=C(Nc1cnc2c(F)cccc2c1)C(=O)N(CCc1ccccc1)Cc1ccncc1]

c)N1-(6-amino-5,6,7,8-tetrahydroquinolin-3-yl)-N2-(3-fluoro-4-(pyridin-3-yl)benzyl)-N2-methyloxalamide:

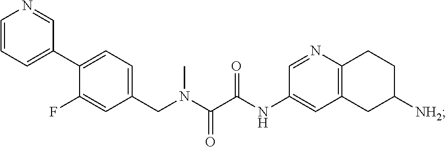 [SMILES: CN(Cc1ccc(-c2cccnc2)c(F)c1)C(=O)C(=O)Nc1cnc2c(c1)CC(N)CC2]

d)N1-cyclopentyl-N1-(3-fluorobenzyl)-N2-(8-fluoroquinolin-3-yl)oxalamide:

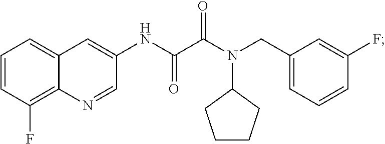 [SMILES: O=C(Nc1cnc2c(F)cccc2c1)C(=O)N(Cc1cccc(F)c1)C1CCCC1]

e)N1-(4-carbamoylbenzyl)-N2-(8-fluoroquinolin-3-yl)-N1-methyloxalamide:

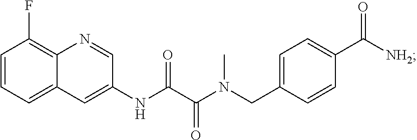 [SMILES: CN(Cc1ccc(C(N)=O)cc1)C(=O)C(=O)Nc1cnc2c(F)cccc2c1]

f)N1-benzyl-N1-methyl-N2-(quinolin-3-yl)oxalamide:

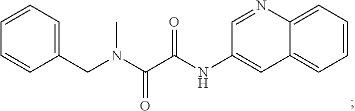 [SMILES: CN(Cc1ccccc1)C(=O)C(=O)Nc1cnc2ccccc2c1]

g)N1-([1,2,4]triazolo[4,3-a]pyridin-3-ylmethyl)-N2-(5-((dimethylamino)methyl)pyridin-3-yl)-N1-methyloxalamide:

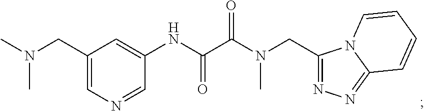 [SMILES: CN(C)Cc1cncc(NC(=O)C(=O)N(C)Cc2nnc3ccccn23)c1]

h)N1-(4-bromo-2-(3-chlorophenoxy)benzyl)-N2-(5-bromo-2-(4-methylpiperazin-1-yl)pyridin-3-yl)-N1-methyloxalamide:

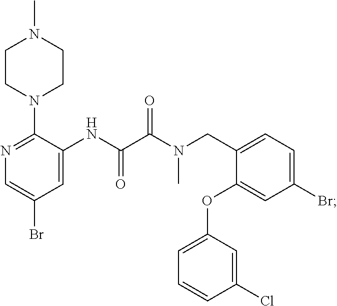 [SMILES: CN1CCN(c2ncc(Br)cc2NC(=O)C(=O)N(C)Cc2ccc(Br)cc2Oc2cccc(Cl)c2)CC1]

i) methyl 4-((N-methyl-2-oxo-2-((5-(trifluoromethyl)pyridin-3-yl)amino)acetamido)methyl)benzoate:

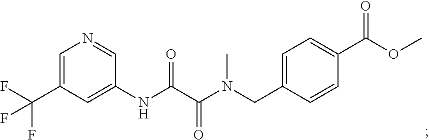 [SMILES: COC(=O)c1ccc(CN(C)C(=O)C(=O)Nc2cncc(C(F)(F)F)c2)cc1]

j)N1-methyl-N1-(2-methylbenzyl)-N2-(2-oxo-5-(trifluoromethyl)-1,2-dihydropyridin-3-yl)oxalamide:

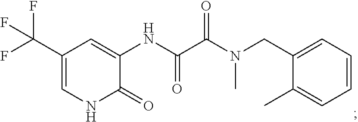 [SMILES: Cc1ccccc1CN(C)C(=O)C(=O)Nc1cc(C(F)(F)F)c[nH]c1=O]

k)N1-(furan-2-ylmethyl)-N1-methyl-N2-(2-oxo-5-(trifluoromethyl)-1,2-dihydropyridin-3-yl)oxalamide:

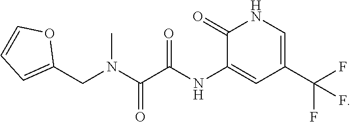 [SMILES: CN(Cc1ccco1)C(=O)C(=O)Nc1cc(C(F)(F)F)c[nH]c1=O]

In an embodiment, provided is a compound of Formula (A) or a pharmaceutically acceptable salt thereof;
wherein:

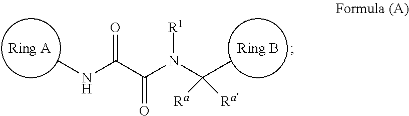

Ring A is selected from the group consisting of:

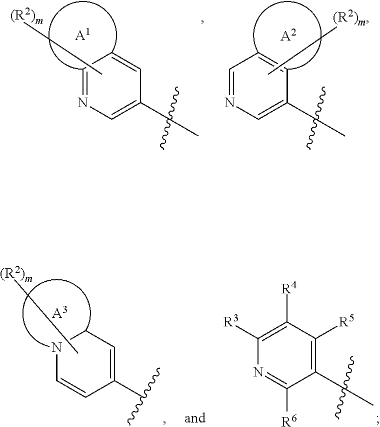

Ring B is selected from the group consisting of C₆-C₁₀ aryl and 5-10 membered heteroaryl, each substituted at any available position with 0, 1, 2 or 3 instances of R7;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₁-C₆ alkyl, —C₂-C₆ heteroalkyl, —C₂-C₆ haloalkyl, —C₃-C₁₀ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each substituted at any available position with 0, 1, 2 or 3 instances of R8; each R² is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C6 alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R⁷ is independently selected from the group consisting of -D, —O, —CN, halo, —SF5, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a7}, —N(R^{a7})₂, —C(═O)R^{a7}, —C(═O)OR^{a7}, —NR^{a7}C(═O)R^{a7}, —NR^{a7}C(═O)OR^{a7}, —C(═O)N(R^{a7})₂, —OC(═O)R^{a7}, —OC(═O)N(R^{a7})₂, —S(═O)R^{a7}, —S(═O)₂R^{a7}, —SR^{a7}, —S(═O)(═NR^{a7})R^{a7}, —NR^{a7}S(═O)₂R^{a7} and —S(═O)₂N(R^{a7})₂, wherein each alkyl, cycloalkyl, heterocyclyl, aryl, heteroaryl, cycloalkylalkyl, heterocyclylalkyl, arylalkyl and heteroarylalkyl of R⁷ is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof);
each R⁸ is independently selected from the group consisting of halo, —O, —CN, —OH, —NH₂, —C₁-C₆ alkyl, —C₁-C₆ haloalkyl, —O(C₁-C₆ alkyl), —O(C₁-C₆ haloalkyl), —NH(C₁-C₆ alkyl), —NH(C₁-C₆ haloalkyl), —N(C₁-C₆ alkyl)₂, —N(C₁-C₆ haloalkyl)₂, —C(O)NH₂, —NHC (O)(C₁-C₆ alkyl), C₃-C₉ cycloalkyl and C₁-C₆ heteroalkyl;
each R^{a} and R^{a}′ are independently selected from H and C₁-C₆ alkyl;
each R^{a2}, R^{a3}, R^{a4}, R^{a5} and R^{a6} is independently selected from the group consisting of H, —C₁-C₆ alkyl, —C₁-C₆ heteroalkyl, C₃—Co cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each Ro is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu) and C₃-C₉ cycloalkyl (e.g., cyclopropyl, cyclobutyl, cyclopentyl, cyclohexyl);
each R^{a7} is independently H; —C₁-C₆ alkyl; —C₁-C₆ haloalkyl; —C₁-C₆ heteroalkyl substituted with 0 or 1 instance of ═O; C₃-C₉ cycloalkyl; or 3-10 membered heterocyclyl substituted with 0 or 1 instances of ═O, -Me or a combination thereof; and
m is 0, 1, 2 or 3; wherein
(i) when R⁴ is —CH₃ then R³ is not H and;
(ii) the compound is not one of compounds a) to k) or a pharmaceutically acceptable salt thereof:
a)N1-benzyl-N2-(8-fluoroquinolin-3-yl)-N1-(2-(2-methoxyethoxy)ethyl)oxalamide:

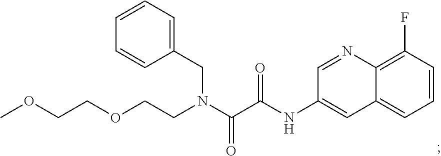 [SMILES: COCCOCCN(Cc1ccccc1)C(=O)C(=O)Nc1cnc2c(F)cccc2c1]

b)N1-(8-fluoroquinolin-3-yl)-N2-phenethyl-N2-(pyridin-4-ylmethyl)oxalamide:

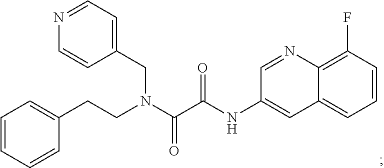 [SMILES: O=C(Nc1cnc2c(F)cccc2c1)C(=O)N(CCc1ccccc1)Cc1ccncc1]

c)N1-(6-amino-5,6,7,8-tetrahydroquinolin-3-yl)-N2-(3-fluoro-4-(pyridin-3-yl)benzyl)-N2-methyloxalamide:

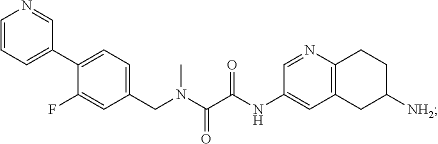 [SMILES: CN(Cc1ccc(-c2cccnc2)c(F)c1)C(=O)C(=O)Nc1cnc2c(c1)CC(N)CC2]

d)N1-cyclopentyl-N1-(3-fluorobenzyl)-N2-(8-fluoroquinolin-3-yl)oxalamide:

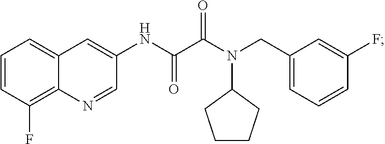 [SMILES: O=C(Nc1cnc2c(F)cccc2c1)C(=O)N(Cc1cccc(F)c1)C1CCCC1]

e)N1-(4-carbamoylbenzyl)-N2-(8-fluoroquinolin-3-yl)-N1-methyloxalamide:

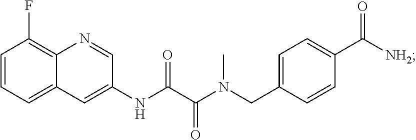 [SMILES: CN(Cc1ccc(C(N)=O)cc1)C(=O)C(=O)Nc1cnc2c(F)cccc2c1]

f)N1-benzyl-N1-methyl-N2-(quinolin-3-yl)oxalamide:

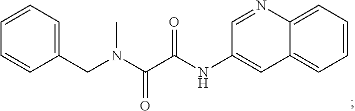 [SMILES: CN(Cc1ccccc1)C(=O)C(=O)Nc1cnc2ccccc2c1]

g)N1-([1,2,4]triazolo[4,3-a]pyridin-3-ylmethyl)-N2-(5-((dimethylamino)methyl)pyridin-3-yl)-N1-methyloxalamide:

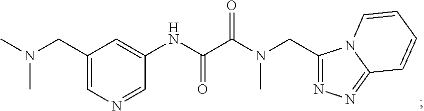 [SMILES: CN(C)Cc1cncc(NC(=O)C(=O)N(C)Cc2nnc3ccccn23)c1]

h)N1-(4-bromo-2-(3-chlorophenoxy)benzyl)-N2-(5-bromo-2-(4-methylpiperazin-1-yl)pyridin-3-yl)-N1-methyloxalamide:

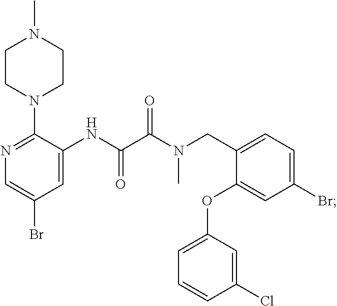 [SMILES: CN1CCN(c2ncc(Br)cc2NC(=O)C(=O)N(C)Cc2ccc(Br)cc2Oc2cccc(Cl)c2)CC1]

i) methyl 4-((N-methyl-2-oxo-2-((5-(trifluoromethyl)pyridin-3-yl)amino)acetamido)methyl)benzoate:

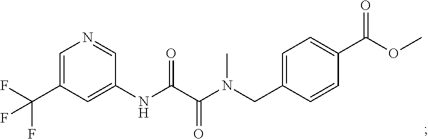 [SMILES: COC(=O)c1ccc(CN(C)C(=O)C(=O)Nc2cncc(C(F)(F)F)c2)cc1]

j)N1-methyl-N1-(2-methylbenzyl)-N2-(2-oxo-5-(trifluoromethyl)-1,2-dihydropyridin-3-yl)oxalamide:

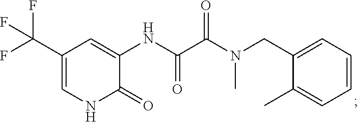 [SMILES: Cc1ccccc1CN(C)C(=O)C(=O)Nc1cc(C(F)(F)F)c[nH]c1=O ; ";"]

k)N1-(furan-2-ylmethyl)-N1-methyl-N2-(2-oxo-5-(trifluoromethyl)-1,2-dihydropyridin-3-yl)oxalamide:

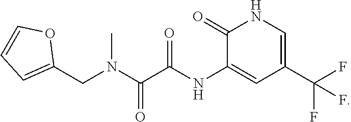 [SMILES: CN(Cc1ccco1)C(=O)C(=O)Nc1cc(C(F)(F)F)c[nH]c1=O]

In one embodiment, provided herein are compounds of Formula (A) or pharmaceutically acceptable salts thereof:
wherein

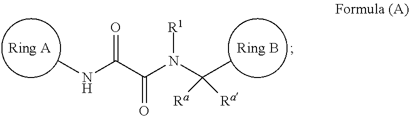

Ring A is selected from the group consisting of:

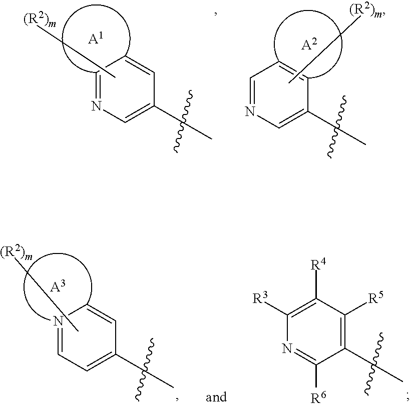

Ring B is selected from the group consisting of C₆-C₁₀ aryl and 5-10 membered heteroaryl, each optionally substituted at any available position;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₂-C₆ alkyl, —C₂-C₆ heteroalkyl, —C₂-C₆ haloalkyl, —C₃-C₉ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position;
each R² is independently selected from the group consisting of halo, ═O, —CN, —C₁-C₆ alkyl, —C₁-C₆ heteroalkyl, —C₁-C₆ haloalkyl, —C₁-C₆ haloalkoxy, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a} and R^{a}′ are independently selected from H and C₁-C₆ alkyl;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R′, —C(═O)OR^{b}, —NR^{b}C(═O)R^{b}, —NR^{b}C(═O)OR′, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)>R^{b} and —S(═O)₂N(R²)₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu).and C₃-C₉ cycloalkyl (e.g., cyclopropyl, cyclobutyl, cyclopentyl, cyclohexyl); and
m is 0, 1, 2 or 3; wherein
when R⁴ is —CH₃ then R³ is not H and;
the compound is not:
a)N1-benzyl-N2-(8-fluoroquinolin-3-yl)-N1-(2-(2-methoxyethoxy)ethyl)oxalamide;
b)N1-(8-fluoroquinolin-3-yl)-N2-phenethyl-N2-(pyridin-4-ylmethyl)oxalamide.

In one embodiment, provided is a compound of Formula (A), or a pharmaceutically acceptable salt thereof, wherein:

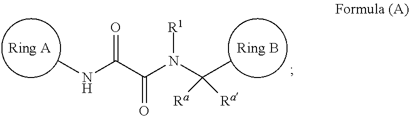

Ring A is selected from the group consisting of:

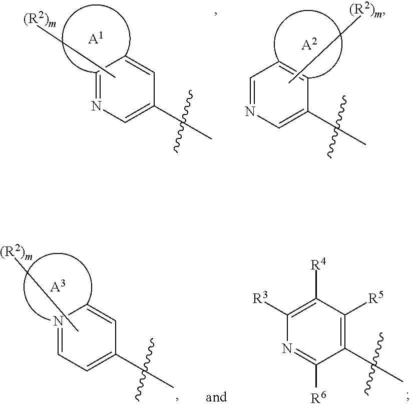

Ring B is selected from the group consisting of C₆-C₁₀ aryl and 5-10 membered heteroaryl, each substituted at any available position with 0, 1, 2 or 3 instances of R7;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₂-C₆ alkyl, —C₂-C₆ heteroalkyl, —C₂-C₆ haloalkyl, —C₃-C₉ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each substituted at any available position with 0, 1, 2 or 3 instances of R8;
   each R² is independently selected from the group consisting of halo, ═O, —CN, —C₁-C₆ alkyl, —C₁-C₆ heteroalkyl, —C₁-C₆ haloalkyl, —C₁-C₆ haloalkoxy, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
   each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
   each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
   each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
   each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R⁷ is independently selected from the group consisting of -D, —O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a7}, —N(R^{a7})₂, —C(═O)R^{a7}, —C(═O)OR^{a7}, —NR^{a7}C(═O)R^{a7}, —NR^{a7}C(═O)OR^{a7}, —C(═O)N(R^{a7})₂, —OC(═O)R^{a7}, —OC(═O)N(R^{a7})₂, —S(═O)R^{a7}, —S(═O)₂R^{a7}, —SR^{a7}, —S(═O)(═NR^{a7})R^{a7}, —NR^{a7}S(═O)₂R^{a7} and —S(═O)₂N(R^{a7})₂, wherein each alkyl, cycloalkyl, heterocyclyl, aryl, heteroaryl, cycloalkylalkyl, heterocyclylalkyl, arylalkyl and heteroarylalkyl of R⁷ is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof);
each R⁸ is independently selected from the group consisting of halo, —O, —CN, —OH, —NH₂, —C₁-C₆ alkyl, —C₁-C₆ haloalkyl, —O(C₁-C₆ alkyl), —O(C₁-C₆ haloalkyl), —NH(C₁-C₆ alkyl), —NH(C₁-C₆ haloalkyl), —N(C₁-C₆ alkyl)₂, —N(C₁-C₆ haloalkyl)₂, C₃-C₉ cycloalkyl and C₁-C₆ heteroalkyl;
each R^{a} and R^{a}′ are independently selected from H and C₁-C₆ alkyl;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu). and C₃-C₉ cycloalkyl (e.g., cyclopropyl, cyclobutyl, cyclopentyl, cyclohexyl).
each R^{a7} is independently H; —C₁-C₆ alkyl; —C₁-C₆ haloalkyl; —C₁-C₆ heteroalkyl substituted with 0 or 1 instance of ═O; C₃-C₉ cycloalkyl; or 3-10 membered heterocyclyl substituted with 0 or 1 instances of ═O, -Me or a combination thereof.
m is 0, 1, 2 or 3; wherein
when R⁴ is —CH₃ then R³ is not H and;
the compound is not:
a)N1-benzyl-N2-(8-fluoroquinolin-3-yl)-N1-(2-(2-methoxyethoxy)ethyl)oxalamide;
b)N1-(8-fluoroquinolin-3-yl)-N2-phenethyl-N2-(pyridin-4-ylmethyl)oxalamide.

As generally defined herein, each R^{a} is independently H or C₁-C₆ alkyl. In some embodiments, each R^{a} is independently H or -Me. In some embodiments, each R^{a} is independently H. In some embodiments, each R^{a} is independently-Me.

As generally defined herein, each R^{a}′ is independently H or C₁-C₆ alkyl. In some embodiments, each R^{a}′ is independently H or -Me. In some embodiments, each Ra′ is independently H. In some embodiments, each R^{a}′ is independently-Me. In some embodiments, each R^{a}′ is -Et.

In some embodiments, R^{a} is H and R^{a}′ is -Me. In some embodiments, R^{a} is H and R^{a}′ is -Me or -Et. In some embodiments, R^{a} is H and R^{a}′ is -Et.

In some embodiments, provided is a compound of Formula (I) or a pharmaceutically acceptable salt thereof;
wherein:

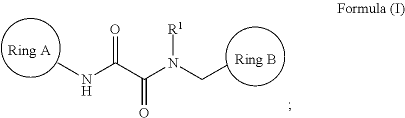

Ring A is selected from the group consisting of:

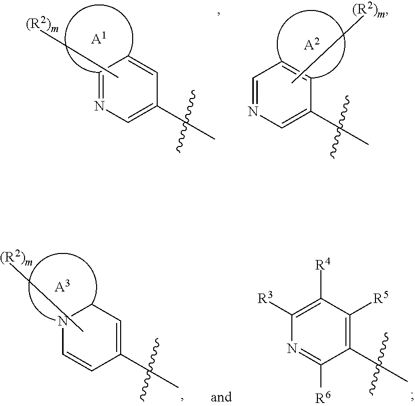

Ring B is selected from the group consisting of C₆-C₁₀ aryl and 5-10 membered heteroaryl, each optionally substituted at any available position;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₁-C₆ alkyl, —C₂-C₆ heteroalkyl, —C₂-C₆ haloalkyl, —C₃-C₁₀ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position;
each R² is independently selected from the group consisting of halo, —O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, -C1-C6 alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, -NRDC(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu) and C₃-C₉ cycloalkyl (e.g., cyclopropyl, cyclobutyl, cyclopentyl, cyclohexyl); and
m is 0, 1, 2 or 3; wherein
(i) when R⁴ is —CH₃ then R³ is not H and;
(ii) the compound is not one of compounds a) to k) or a pharmaceutically acceptable salt thereof:
a)N1-benzyl-N2-(8-fluoroquinolin-3-yl)-N1-(2-(2-methoxyethoxy)ethyl)oxalamide:

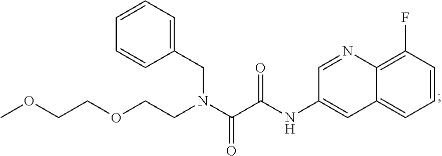 [SMILES: COCCOCCN(Cc1ccccc1)C(=O)C(=O)Nc1cnc2c(F)cccc2c1]

b)N1-(8-fluoroquinolin-3-yl)-N2-phenethyl-N2-(pyridin-4-ylmethyl)oxalamide:

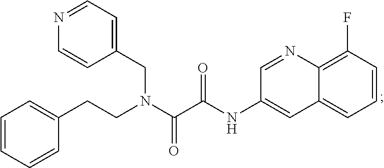 [SMILES: O=C(Nc1cnc2c(F)cccc2c1)C(=O)N(CCc1ccccc1)Cc1ccncc1]

c)N1-(6-amino-5,6,7,8-tetrahydroquinolin-3-yl)-N2-(3-fluoro-4-(pyridin-3-yl)benzyl)-N2-methyloxalamide:

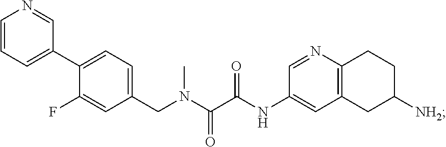 [SMILES: CN(Cc1ccc(-c2cccnc2)c(F)c1)C(=O)C(=O)Nc1cnc2c(c1)CC(N)CC2]

d)N1-cyclopentyl-N1-(3-fluorobenzyl)-N2-(8-fluoroquinolin-3-yl)oxalamide:

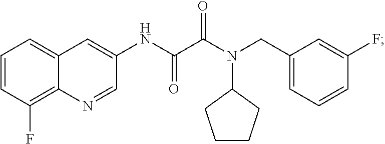 [SMILES: O=C(Nc1cnc2c(F)cccc2c1)C(=O)N(Cc1cccc(F)c1)C1CCCC1]

e)N1-(4-carbamoylbenzyl)-N2-(8-fluoroquinolin-3-yl)-N1-methyloxalamide:

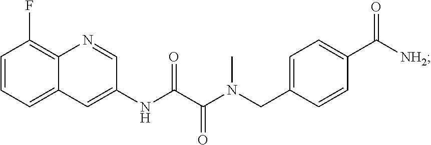 [SMILES: CN(Cc1ccc(C(N)=O)cc1)C(=O)C(=O)Nc1cnc2c(F)cccc2c1]

f)N1-benzyl-N1-methyl-N2-(quinolin-3-yl)oxalamide:

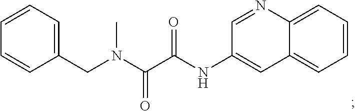 [SMILES: CN(Cc1ccccc1)C(=O)C(=O)Nc1cnc2ccccc2c1]

g)N1-([1,2,4]triazolo[4,3-a]pyridin-3-ylmethyl)-N2-(5-((dimethylamino)methyl)pyridin-3-yl)-N1-methyloxalamide:

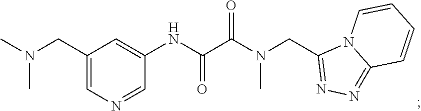 [SMILES: CN(C)Cc1cncc(NC(=O)C(=O)N(C)Cc2nnc3ccccn23)c1]

h)N1-(4-bromo-2-(3-chlorophenoxy)benzyl)-N2-(5-bromo-2-(4-methylpiperazin-1-yl)pyridin-3-yl)-N1-methyloxalamide:

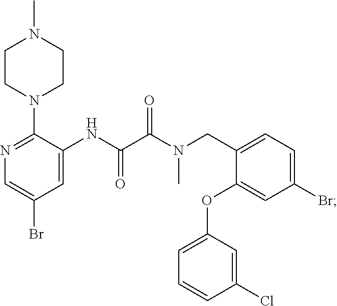 [SMILES: CN1CCN(c2ncc(Br)cc2NC(=O)C(=O)N(C)Cc2ccc(Br)cc2Oc2cccc(Cl)c2)CC1]

i) methyl 4-((N-methyl-2-oxo-2-((5-(trifluoromethyl)pyridin-3-yl)amino)acetamido)methyl)benzoate:

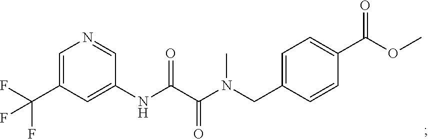 [SMILES: COC(=O)c1ccc(CN(C)C(=O)C(=O)Nc2cncc(C(F)(F)F)c2)cc1]

j)N1-methyl-N1-(2-methylbenzyl)-N2-(2-oxo-5-(trifluoromethyl)-1,2-dihydropyridin-3-yl)oxalamide:

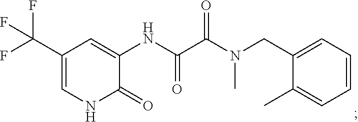 [SMILES: Cc1ccccc1CN(C)C(=O)C(=O)Nc1cc(C(F)(F)F)c[nH]c1=O]

k)N1-(furan-2-ylmethyl)-N1-methyl-N2-(2-oxo-5-(trifluoromethyl)-1,2-dihydropyridin-3-yl)oxalamide:

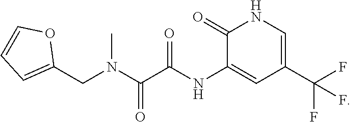 [SMILES: CN(Cc1ccco1)C(=O)C(=O)Nc1cc(C(F)(F)F)c[nH]c1=O]

In some embodiments, provided is a compound of Formula (I) or a pharmaceutically acceptable salt thereof;
wherein:

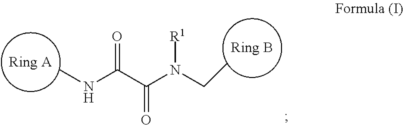

Ring A is selected from the group consisting of:

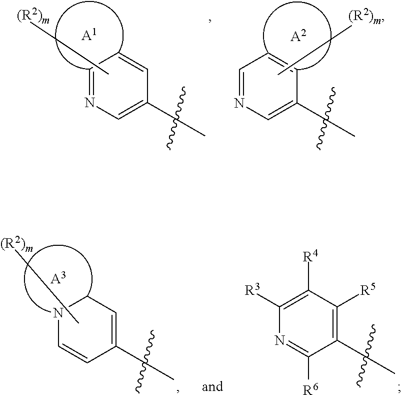

Ring B is selected from the group consisting of C₆-C₁₀ aryl and 5-10 membered heteroaryl, each optionally substituted at any available position;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₂-C₆ alkyl, —C₂-C₆ heteroalkyl, —C₂-C₆ haloalkyl, —C₃-C₉ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position;
each R² is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, -C1-C6 alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R′, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)>R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu) and C₃-C₉ cycloalkyl (e.g., cyclopropyl, cyclobutyl, cyclopentyl, cyclohexyl); and
m is 0, 1, 2 or 3; wherein
when R⁴ is —CH₃ then R³ is not H and;
the compound is not:
N1-benzyl-N2-(8-fluoroquinolin-3-yl)-N1-(2-(2-methoxyethoxy)ethyl)oxalamide;
N1-(8-fluoroquinolin-3-yl)-N2-phenethyl-N2-(pyridin-4-ylmethyl)oxalamide.

In some embodiments, the compounds of Formula (A) are of Formula (A′):

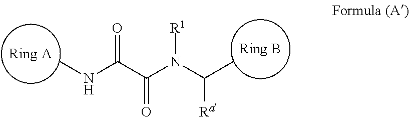

wherein Ring A, Ra′, Ring B and R¹ are as defined in any of the embodiments described herein. In some embodiments, the stereochemistry at the center connected to R^{a}′ is(S). In some embodiments, the stereochemistry at the center connected to R^{a}′ is (R).

In some embodiments, the compounds of Formula (A) are of Formula (I):

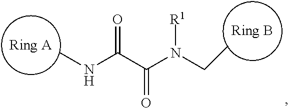

wherein Ring A, Ra, Ra′, Ring B and R¹ are as defined in any of the embodiments described herein.

As generally defined herein, Ring A is selected from the group consisting of:

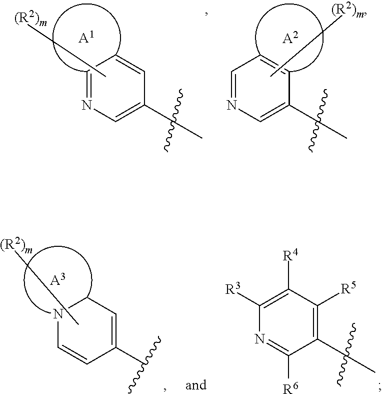

wherein
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R², R³, R₄, R⁵, R6 and m are as defined in any of the embodiments herein.

In one embodiment, rings A¹ and A² are each independently a 5-6 membered carbocyclyl, a 5-6 membered heterocyclyl containing 1, 2 or 3 heteroatoms selected from the group consisting of O, N, S or oxidized forms thereof, a 5-6 membered heteroaryl containing 1, 2 or 3 heteroatoms selected from the group consisting of O, N, S or oxidized forms thereof or a phenyl.

In one embodiment, each ring A³ is independently a 5-6 membered heterocyclyl or 5-6 membered heteroaryl, wherein the heterocyclyl and heteroaryl contain at least one nitrogen atom and 0, 1 or 2 additional heteroatoms selected from the group consisting of N, O or S or oxidized forms thereof.

In one embodiment, Ring A is

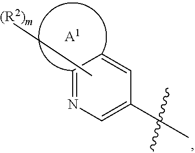

wherein A¹, R² and m are as defined in any of the embodiments described herein.

In one embodiment, Ring A is

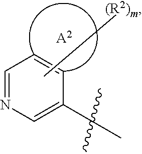

wherein A², R² and m are as defined in any of the embodiments described herein.

In one embodiment, Ring A is

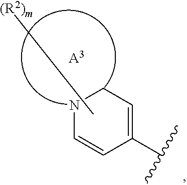

wherein A³, R² and m are as defined in any of the embodiments described herein.

In some embodiments, Ring A is selected from the group consisting of:

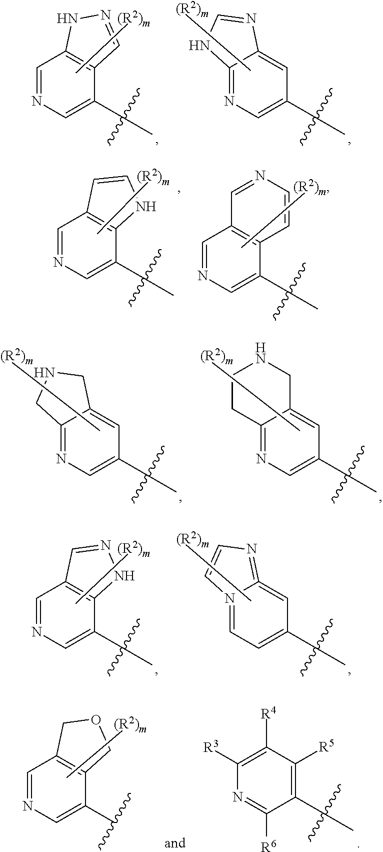

wherein R², R³, R₄, R⁵, Re and m are as defined in any of the embodiments described herein.

In some embodiments, Ring A is selected from the group consisting of:

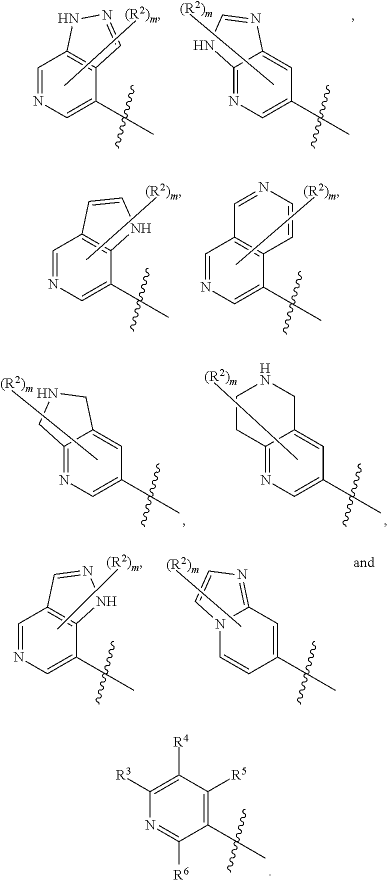

wherein R², R³, R₄, R⁵, R6 and m are as defined in any of the embodiments described herein.

In some embodiments, Ring A is selected from the group consisting of:

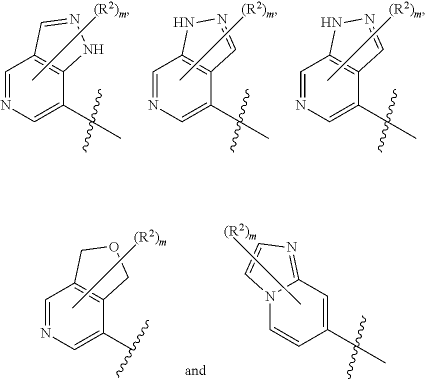

wherein R² and m are as defined in any of the embodiments described herein.

In some embodiments, Ring A is selected from the group consisting of:

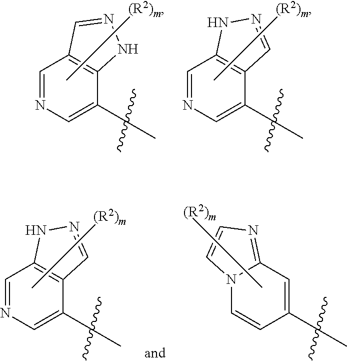

wherein R² and m are as defined in any of the embodiments described herein.

In some embodiments, Ring A is selected from the group consisting of:

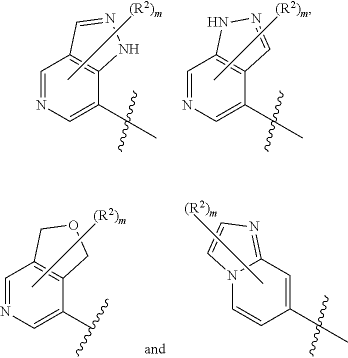

wherein R² and m are as defined in any of the embodiments described herein.

In some embodiments, Ring A is selected from the group consisting of:

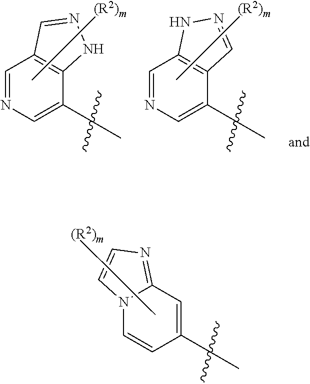

wherein R² and m are as defined in any of the embodiments described herein.

In some embodiments, Ring A is selected from the group consisting of:

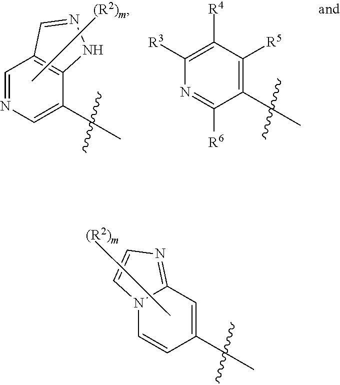

wherein R², R³, R₄, R⁵, Re and m are as defined in any of the embodiments described herein.

In some embodiments, Ring A is selected from the group consisting of:

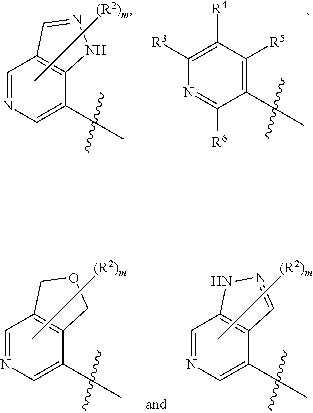

In some embodiments, Ring A is selected from the group consisting of:

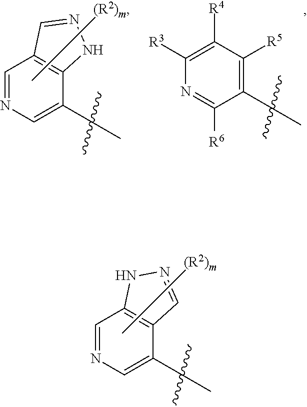

wherein R², R³, R₄, R⁵, R6 and m are as defined in any of the embodiments described herein.

In some embodiments, Ring A is selected from the group consisting of:

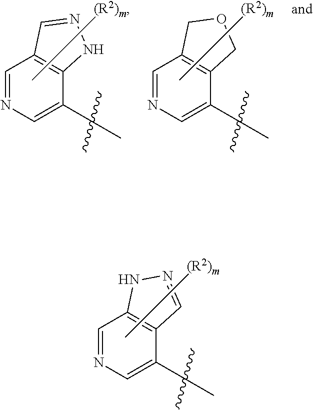

wherein R² and m are as defined in any of the embodiments described herein.

In some embodiments, Ring A is selected from the group consisting of:

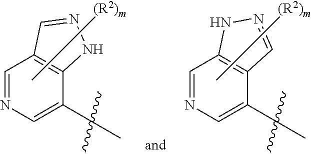

wherein R² and m are as defined in any of the embodiments described herein.

In some embodiments, Ring A is selected from the group consisting of:

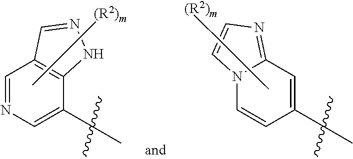

wherein R² and m are as defined in any of the embodiments described herein.

In some embodiments, Ring A is:

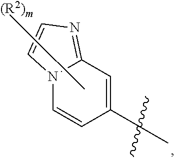

wherein R² and m are as defined in any of the embodiments described herein.

In some embodiments, Ring A is:

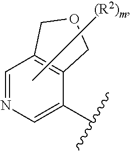

wherein R² and m are as defined in any of the embodiments described herein.

In some embodiments, Ring A is:

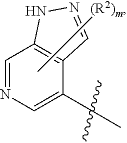

wherein R² and m are as defined in any of the embodiments described herein.

In some embodiments, Ring A is:

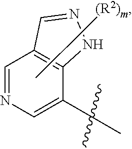

wherein R² and m are as defined in any of the embodiments described herein.

In some embodiments, Ring A is:

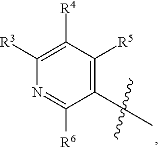

wherein R³, R₄, R⁵ and R⁶ are as defined in any of the embodiments described herein.

In some embodiments, Ring A is:

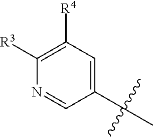

wherein R³ and R⁴ are as defined in any of the embodiments described herein.

As generally defined herein, m is 0, 1, 2 or 3. In some embodiments, m is 0, 1 or 2.

In some embodiments, m is 0 or 1. In some embodiments, m is 1 or 2.

In some embodiments, m is 0.

In some embodiments, m is 1.

In some embodiments, m is 2.

In some embodiments, m is 3.

In some embodiments, Ring A is selected from the group consisting of:

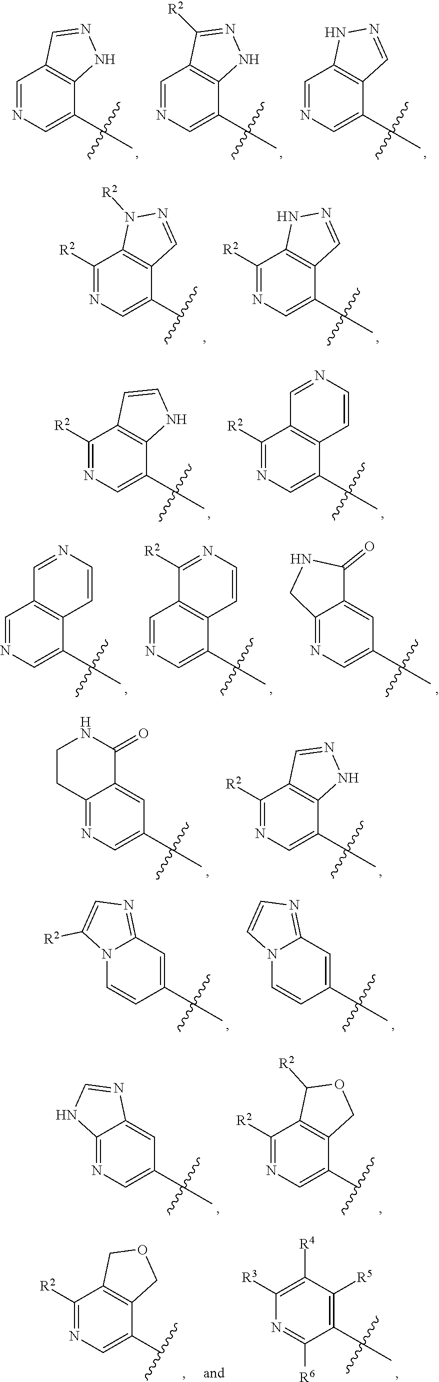

wherein R², R³, R₄, R⁵ and R⁶ are as defined in any of the embodiments described herein.

In some embodiments, Ring A is selected from the group consisting of:

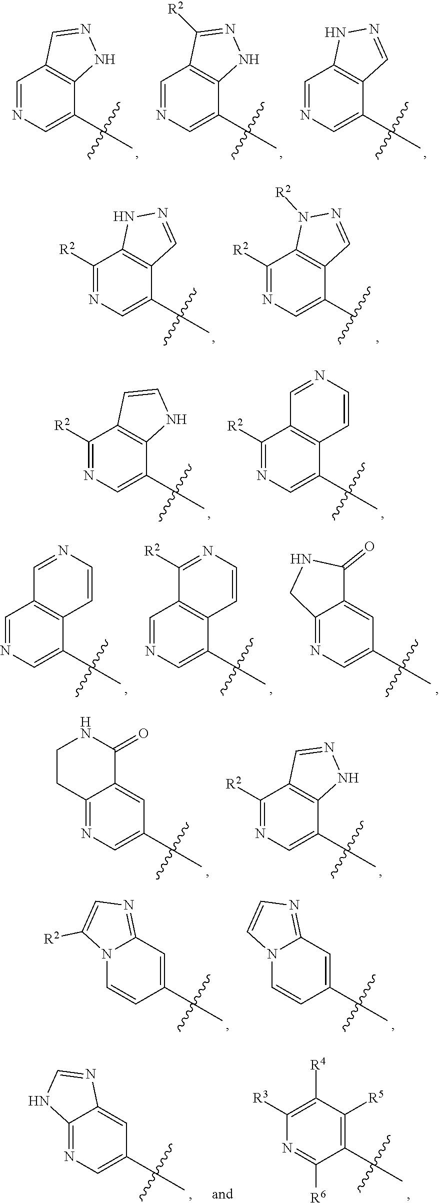

wherein R², R³, R₄, R⁵ and R⁶ are as defined in any of the embodiments described herein.

In some embodiments, Ring A is selected from the group consisting of:

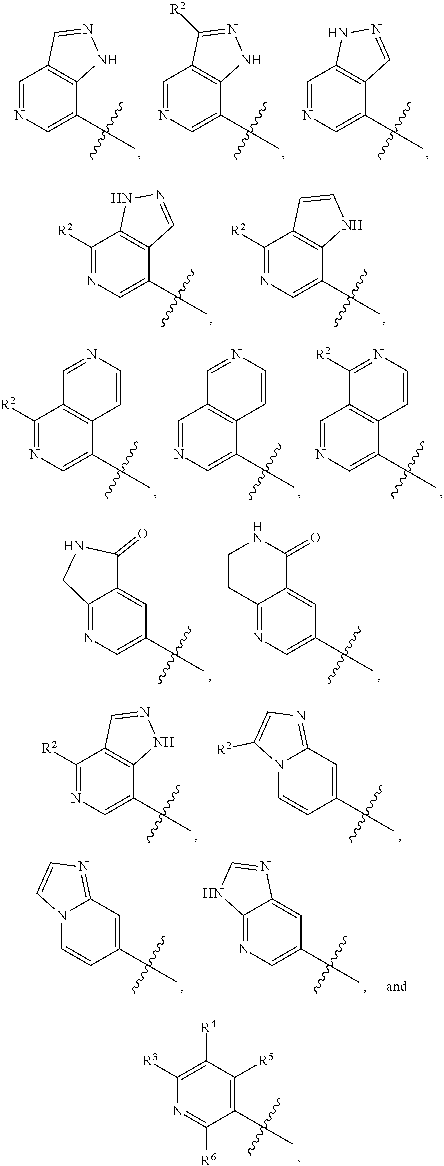

wherein R², R³, R₄, R⁵ and R^{e} are as defined in any of the embodiments described herein.

In some embodiments, Ring A is selected from the group consisting of:

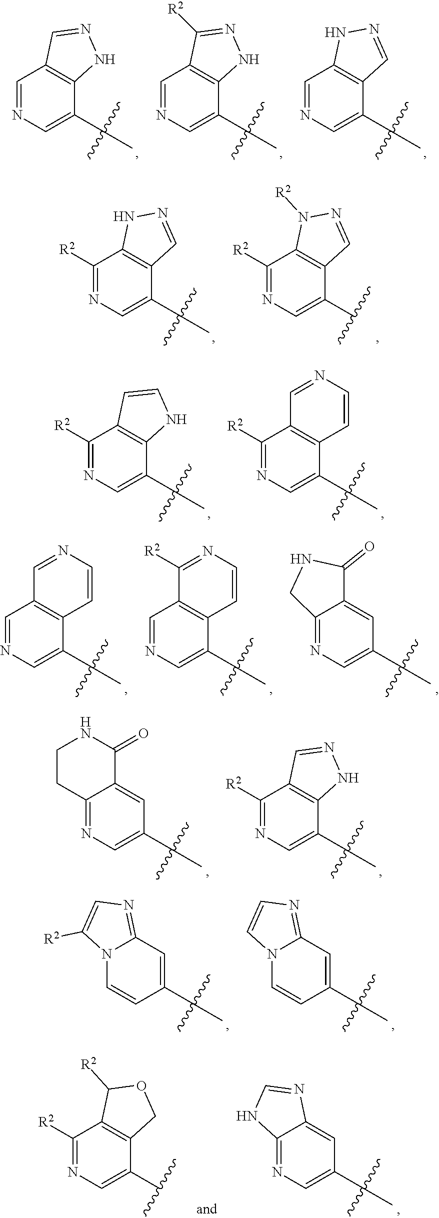

wherein R² is as defined in any of the embodiments described herein.

In some embodiments, Ring A is selected from the group consisting of:

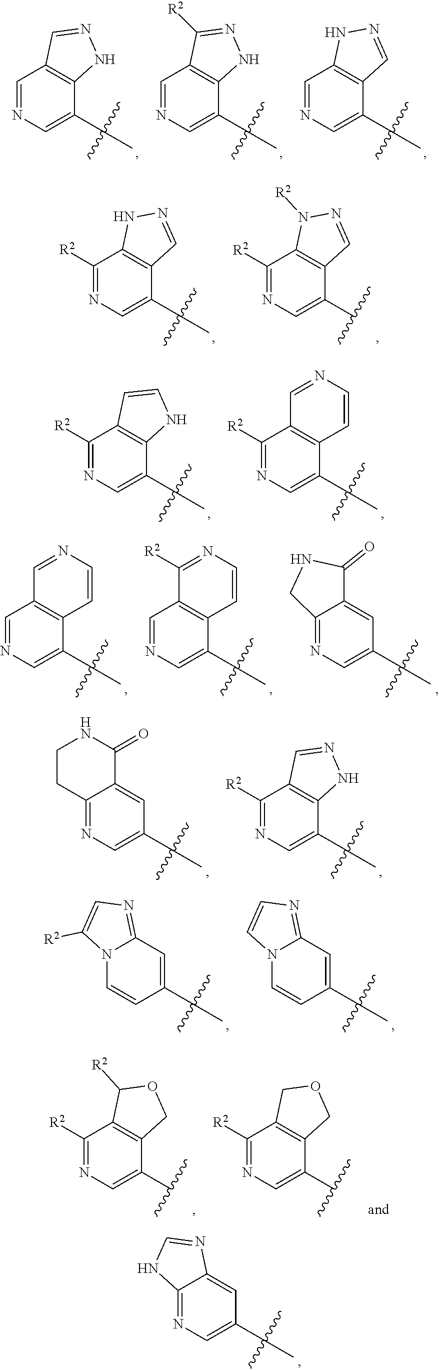

wherein R² is as defined in any of the embodiments described herein.

In some embodiments, Ring A is selected from the group consisting of:

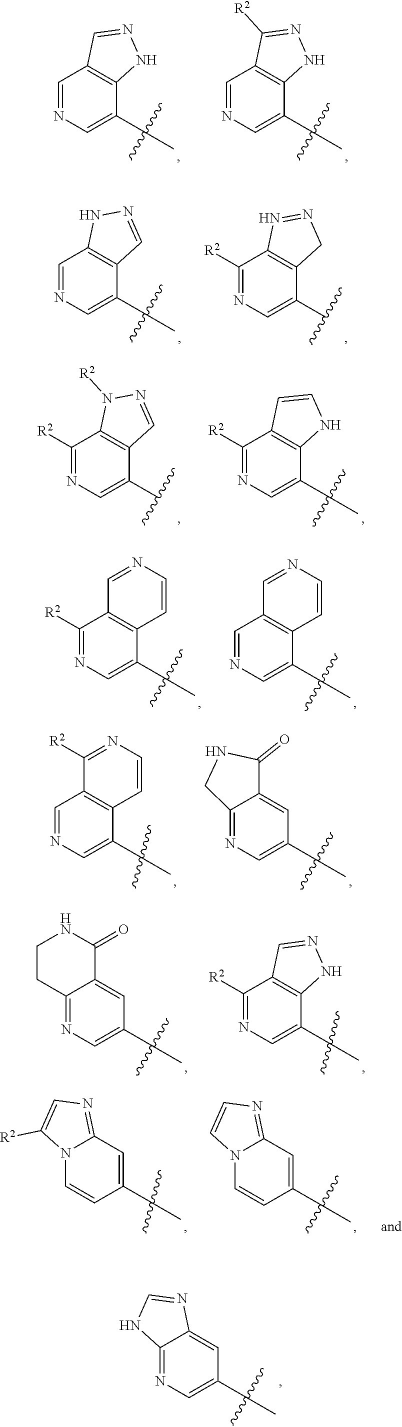

wherein R² is as defined in any of the embodiments described herein.

In some embodiments, Ring A is selected from the group consisting of:

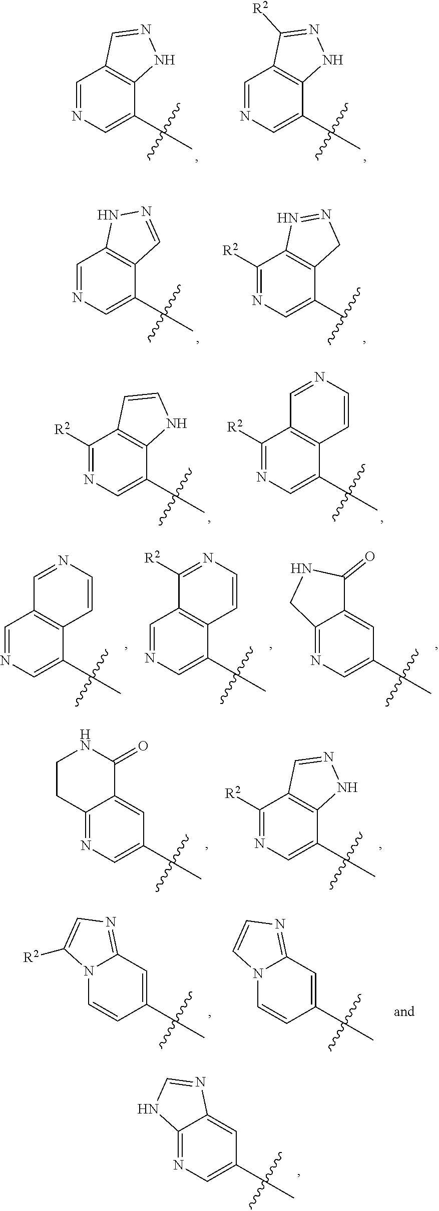

wherein R² is as defined in any of the embodiments described herein.

In some embodiments, Ring A is selected from the group consisting of:

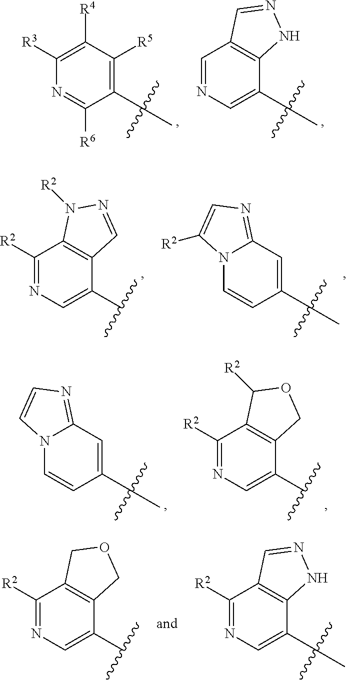

wherein R², R³, R₄, R⁵ and R^{e} are as defined in any of the embodiments described herein.

In some embodiments, Ring A is selected from the group consisting of:

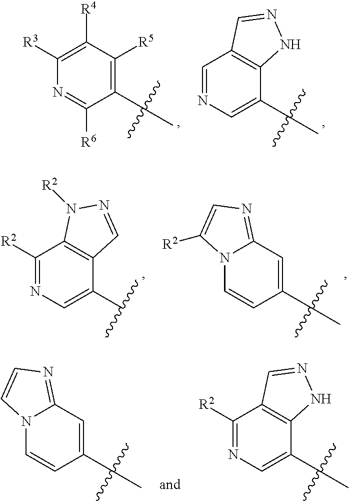

wherein R², R³, R₄, R⁵ and R^{e} are as defined in any of the embodiments described herein.

In some embodiments, Ring A is selected from the group consisting of:

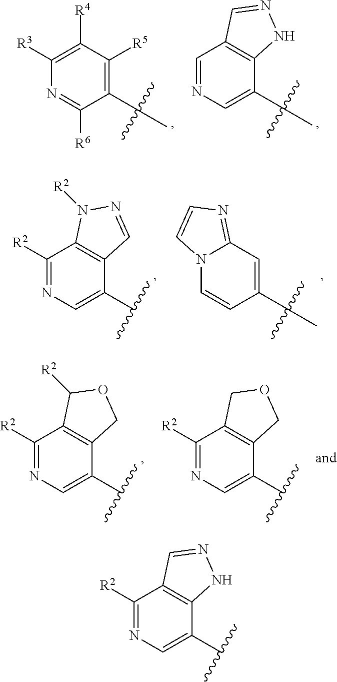

wherein R², R³, R₄, R⁵ and R⁶ are as defined in any of the embodiments described herein.

In some embodiments, Ring A is selected from the group consisting of:

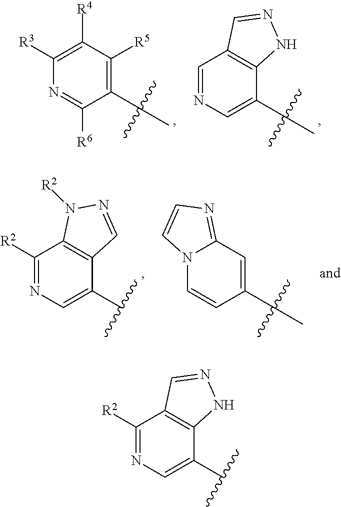

wherein R², R³, R₄, R⁵ and R⁶ are as defined in any of the embodiments described herein. In some embodiments, Ring A is selected from the group consisting of:

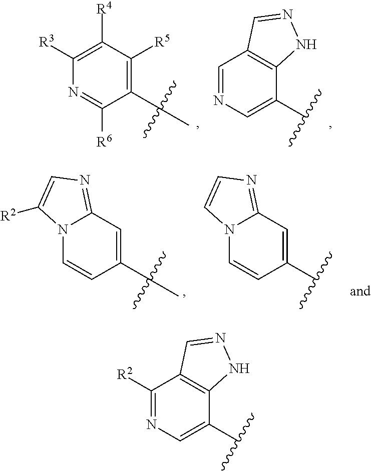

wherein R², R³, R4, R5 and R^{e} are as defined in any of the embodiments described herein.

In some embodiments, Ring A is selected from the group consisting of:

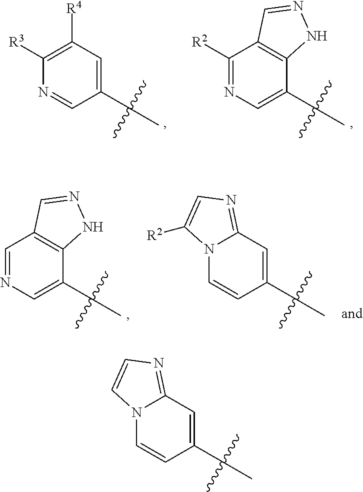

wherein R², R³ and R⁴ are as defined in any of the embodiments described herein.

In some embodiments, Ring A is selected from the group consisting of:

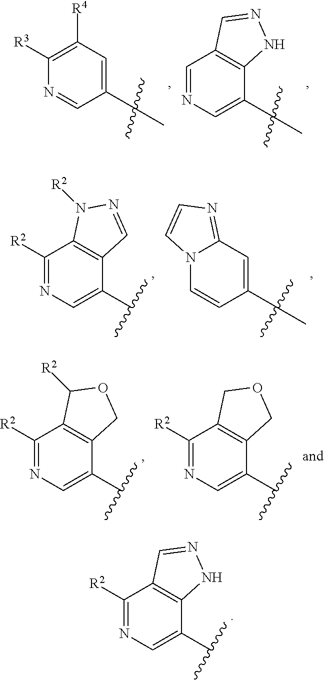

wherein R², R³, R₄, R⁵ and R⁶ are as defined in any of the embodiments described herein.

In some embodiments, Ring A is selected from the group consisting of:

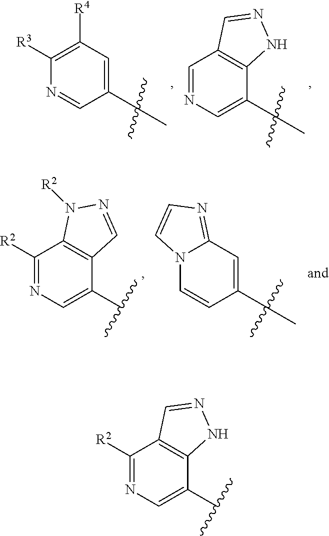

wherein R², R³, R₄, R⁵ and R6 are as defined in any of the embodiments described herein.

In some embodiments, Ring A is selected from the group consisting of:

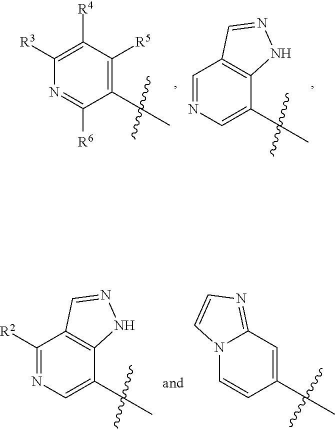

wherein R², R³, R₄, R⁵ and R⁶ are as defined in any of the embodiments described herein.

In some embodiments, Ring A is selected from the group consisting of:

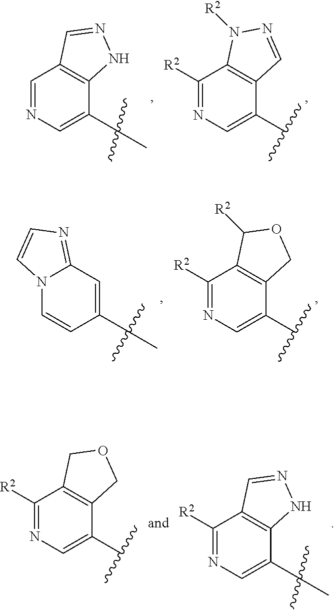

In some embodiments, Ring A is selected from the group consisting of:

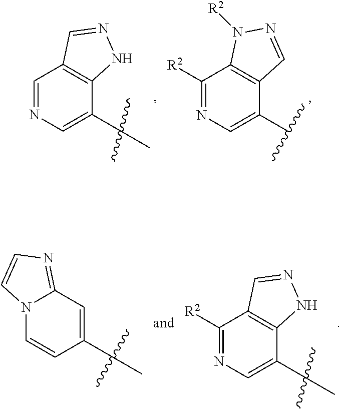

In some embodiments, Ring A is selected from the group consisting of:

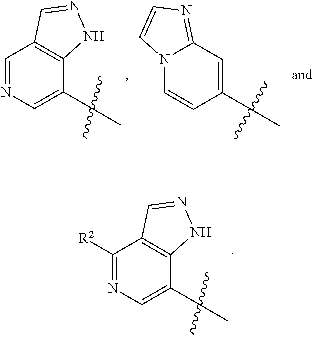

In some embodiments, Ring A is selected from the group consisting of:

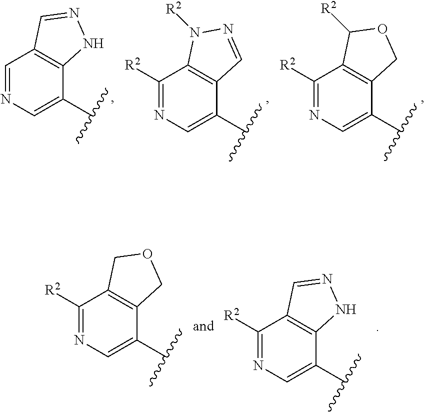

In some embodiments, Ring A is selected from the group consisting of:

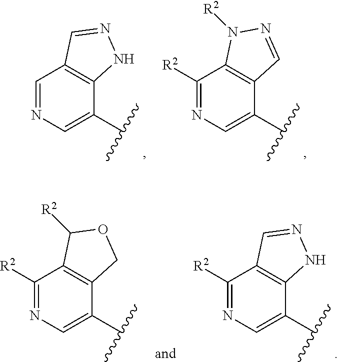

In some embodiments, Ring A is selected from the group consisting of:

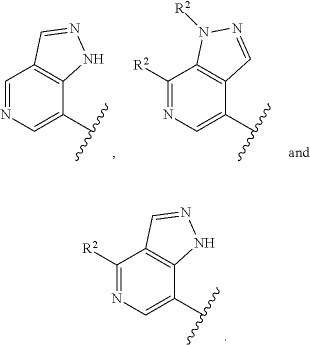

In some embodiments, Ring A is selected from the group consisting of:

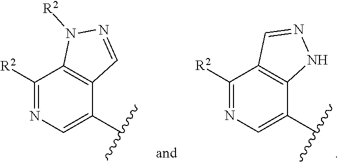

In some embodiments, Ring A is selected from the group consisting of:

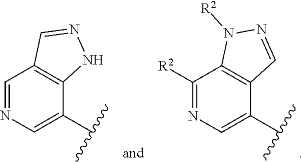

In some embodiments, Ring A is selected from the group consisting of:

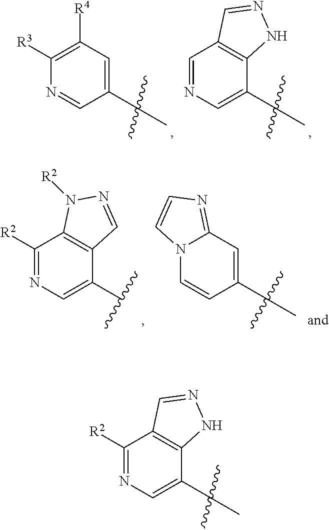

wherein R², R³ and R⁴ are as defined in any of the embodiments described herein.

In some embodiments, Ring A is selected from the group consisting of:

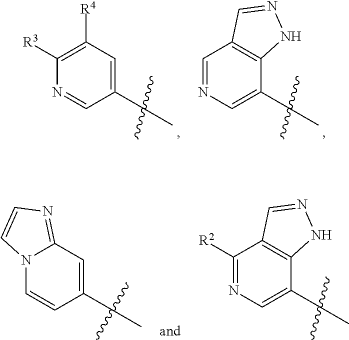

wherein R², R³ and R⁴ are as defined in any of the embodiments described herein.

In some embodiments, Ring A is

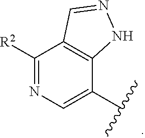

In some embodiments, Ring A is

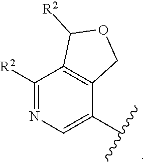

In some embodiments, Ring A is

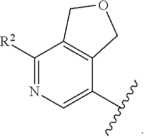

In some embodiments, Ring A is

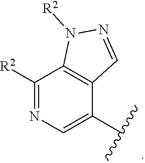

In some embodiments, Ring A is

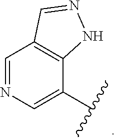 [SMILES: *c1cncc2cn[nH]c12]

In some embodiments, Ring A is

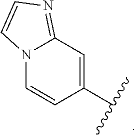 [SMILES: *c1ccn2ccnc2c1]

In some embodiments, Ring A is selected from the group consisting of:

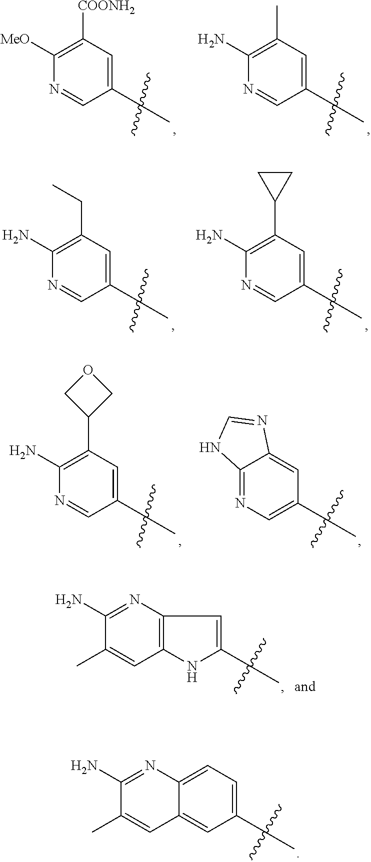

In some embodiments, Ring A is selected from the group consisting of:

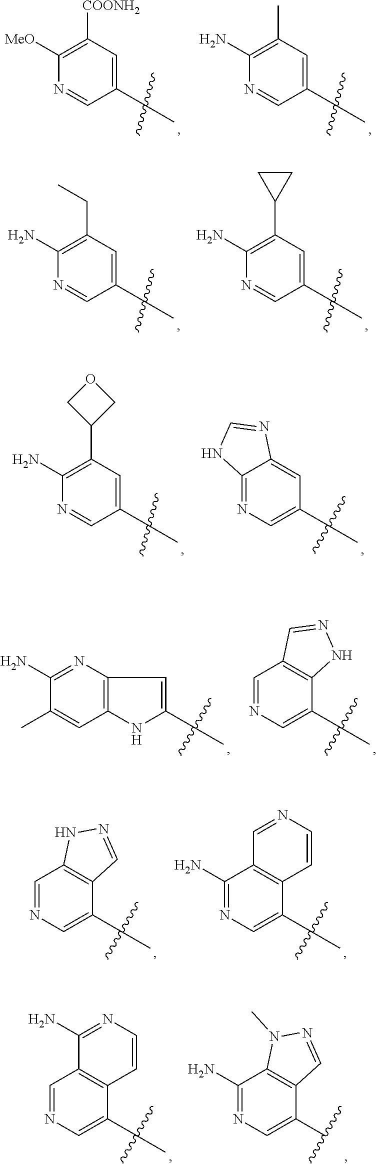

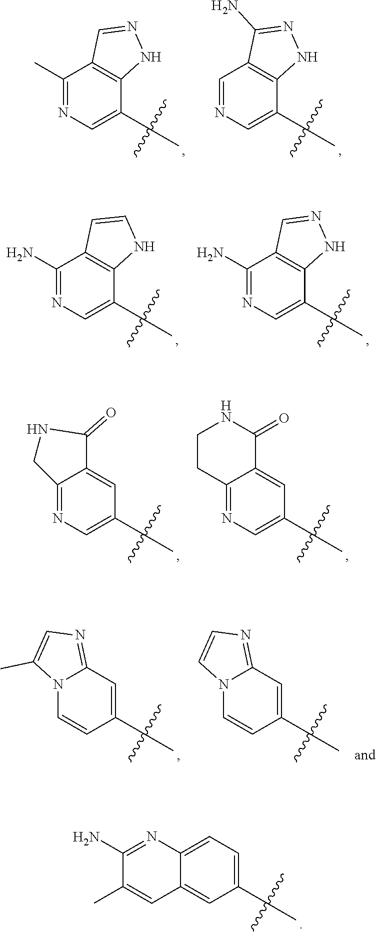

In some embodiments, Ring A is selected from the group consisting of:

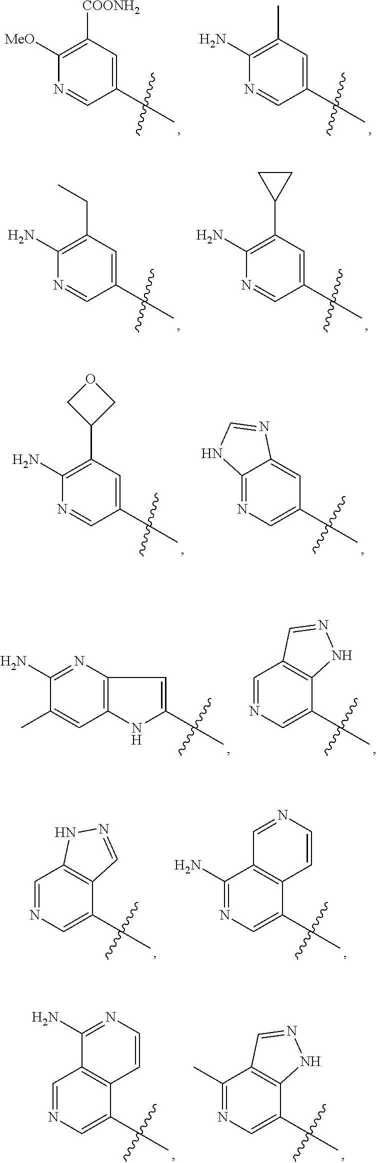

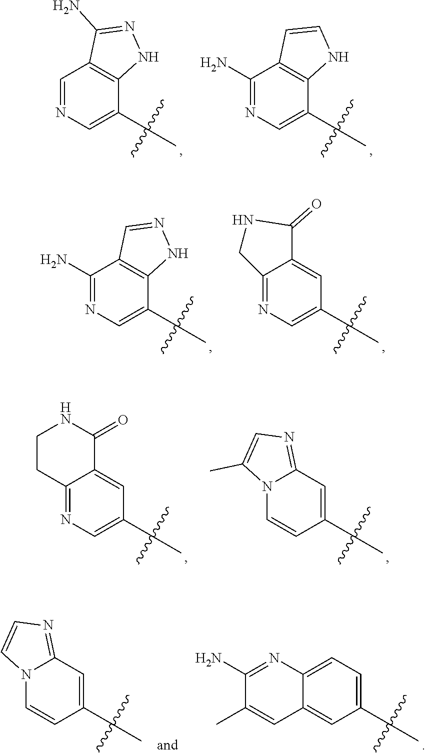

In some embodiments, Ring A is selected from the group consisting of:

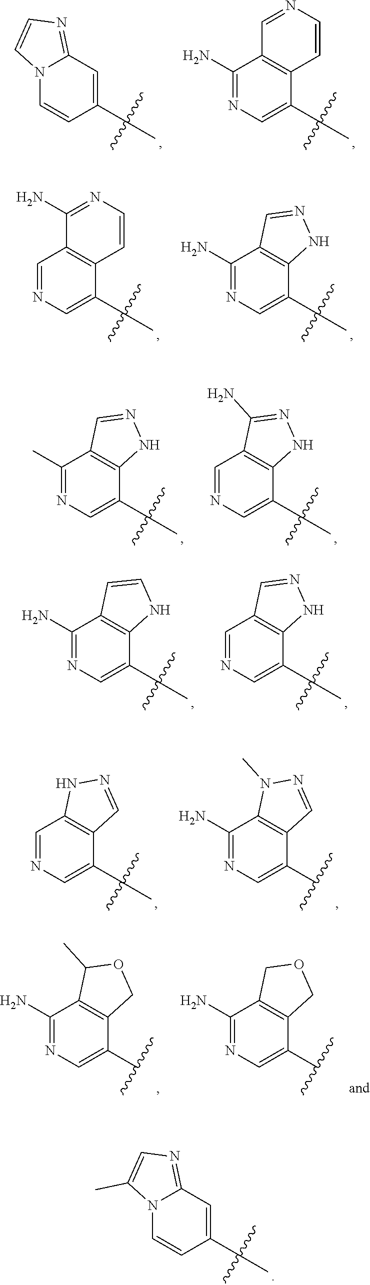

In some embodiments, Ring A is selected from the group consisting of:

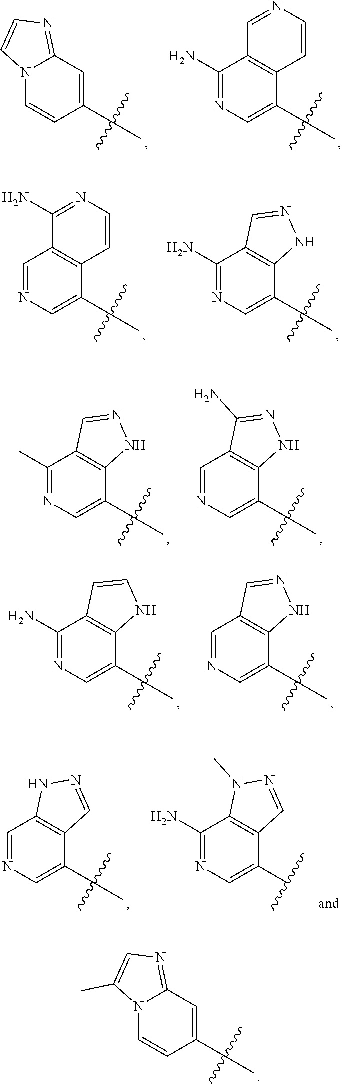

In some embodiments, Ring A is selected from the group consisting of:

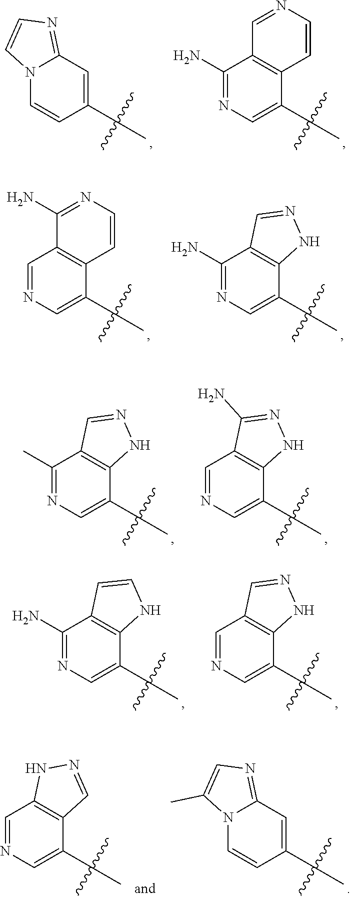

In some embodiments, Ring A is selected from the group consisting of:

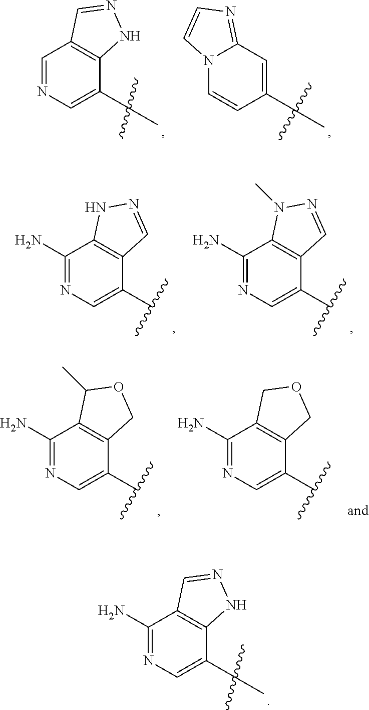

In some embodiments, Ring A is selected from the group consisting of:

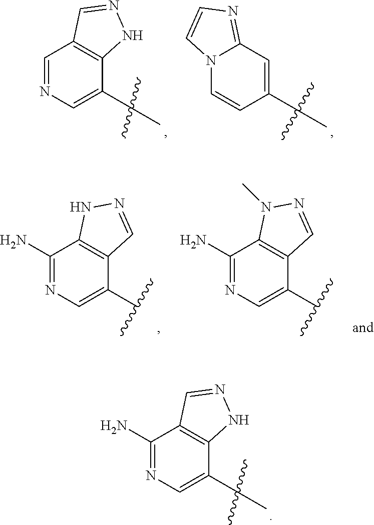 [SMILES: *c1cncc2cn[nH]c12 ; *c1ccn2ccnc2c1 ; *c1cnc(N)c2[nH]ncc12 ; *c1cnc(N)c2c1cnn2C ; *c1cnc(N)c2cn[nH]c12]

In some embodiments, King A is selected from the group consisting of:

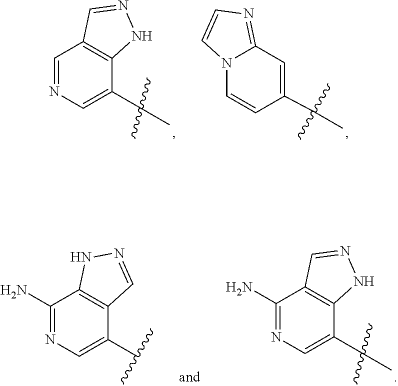 [SMILES: *c1cncc2cn[nH]c12 ; *c1ccn2ccnc2c1 ; *c1cnc(N)c2[nH]ncc12 ; *c1cnc(N)c2cn[nH]c12]

In some embodiments, Ring A is selected from the group consisting of:

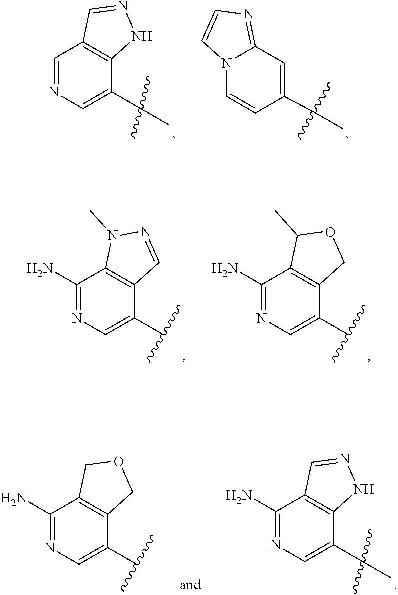 [SMILES: *c1cncc2cn[nH]c12 ; *c1ccn2ccnc2c1 ; *c1cnc(N)c2c1cnn2C ; *c1cnc(N)c2c1COC2C ; *c1cnc(N)c2c1COC2 ; *c1cnc(N)c2cn[nH]c12]

In some embodiments, Ring A is selected from the group consisting of:

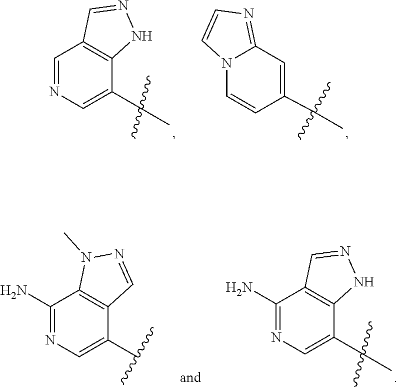 [SMILES: *c1cncc2cn[nH]c12 ; *c1ccn2ccnc2c1 ; *c1cnc(N)c2c1cnn2C ; *c1cnc(N)c2cn[nH]c12]

In some embodiments, Ring A is selected from the group consisting of:

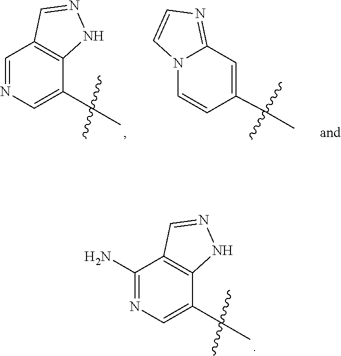 [SMILES: *c1cncc2cn[nH]c12 ; *c1ccn2ccnc2c1 ; *c1cnc(N)c2cn[nH]c12]

In some embodiments, Ring A is selected from the group consisting of:

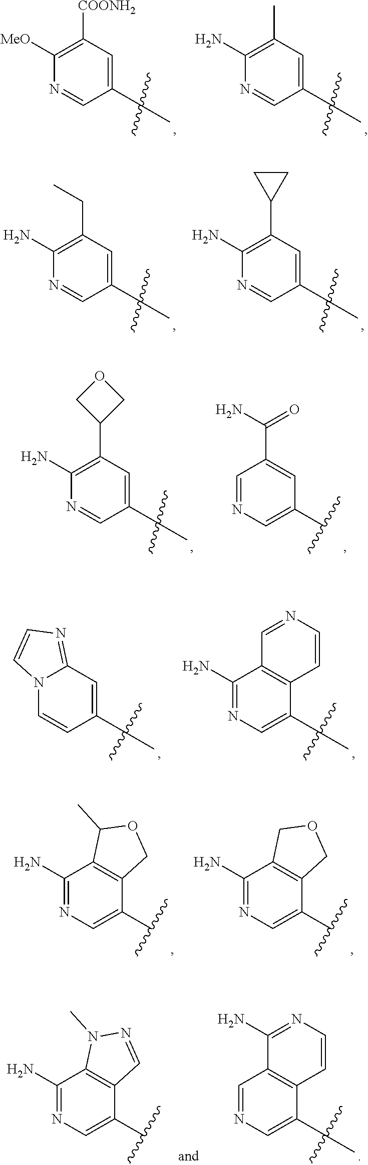

In some embodiments, Ring A is selected from the group consisting of:

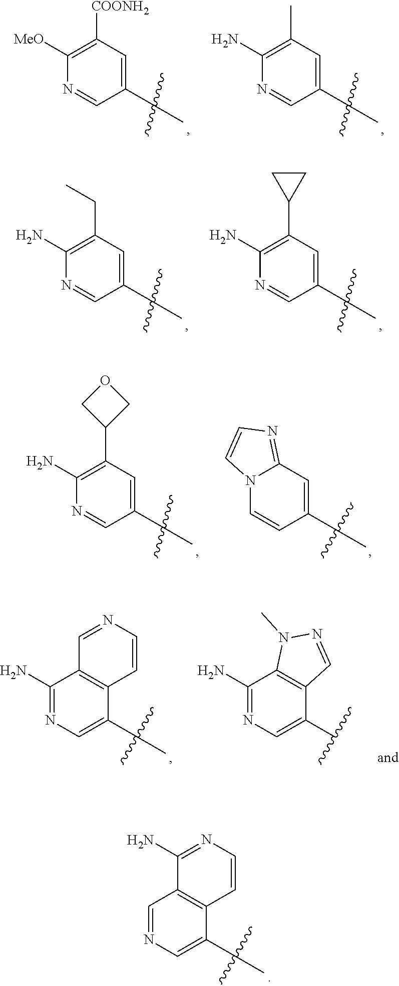

In some embodiments, Ring A is selected from the group consisting of:

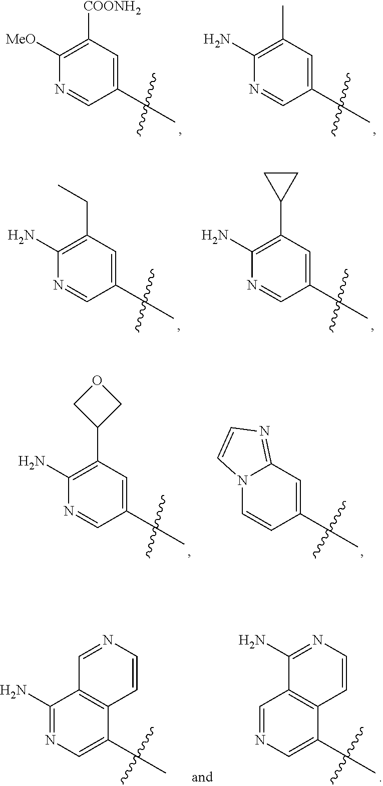

In certain embodiments, Ring A is selected from the group consisting of:

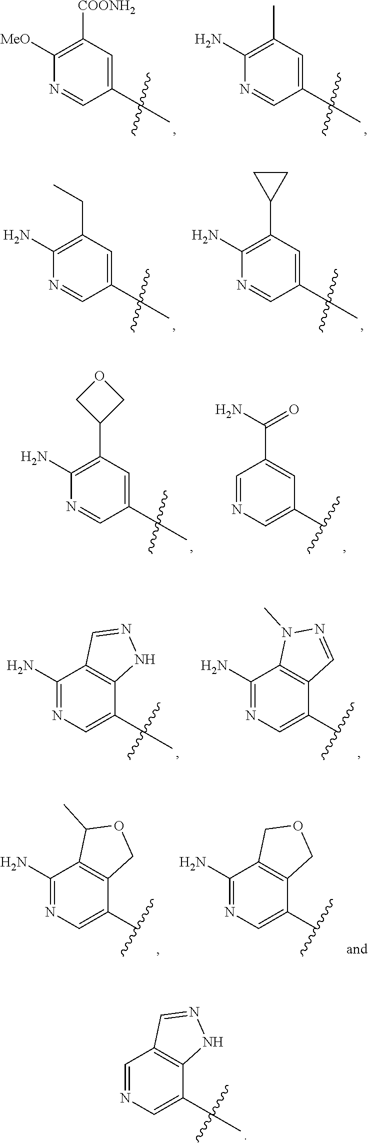

In certain embodiments, Ring A is selected from the group consisting of:

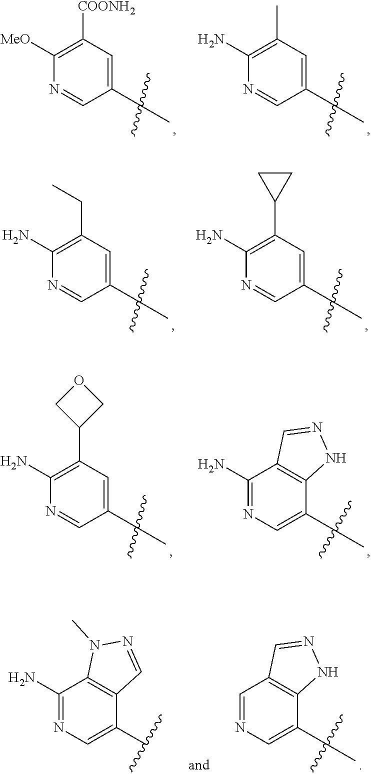

In certain embodiments, Ring A is selected from the group consisting of:

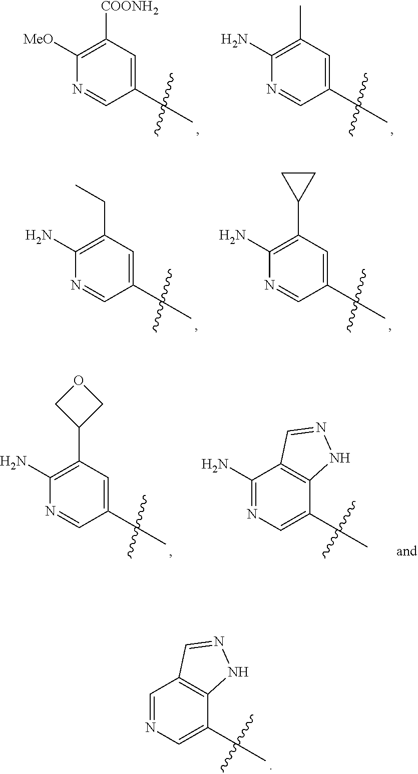

In some embodiments, Ring A is selected from the group consisting of:

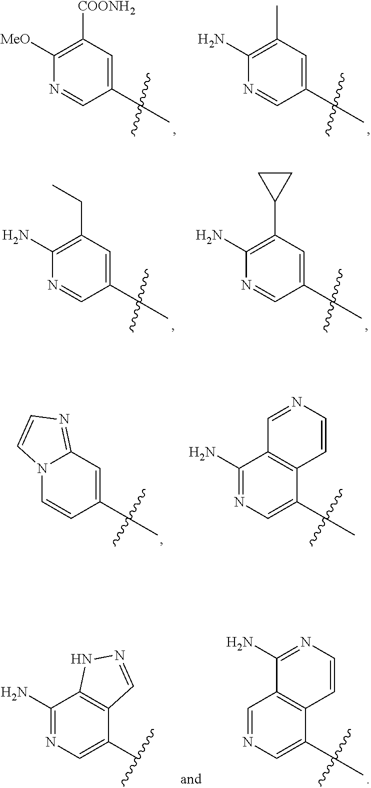

In some embodiments, Ring A is selected from the group consisting of:

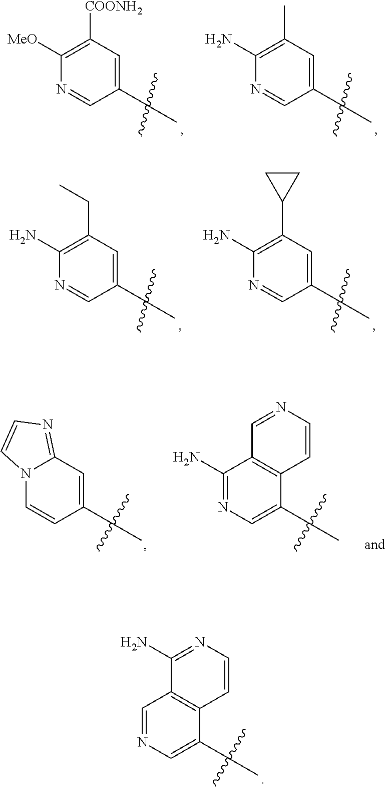

In certain embodiments, Ring A is selected from the group consisting of:

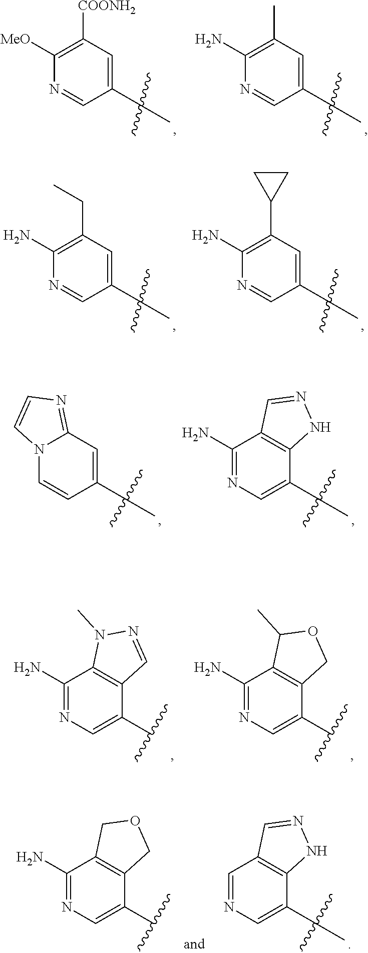

In certain embodiments, Ring A is selected from the group consisting of:

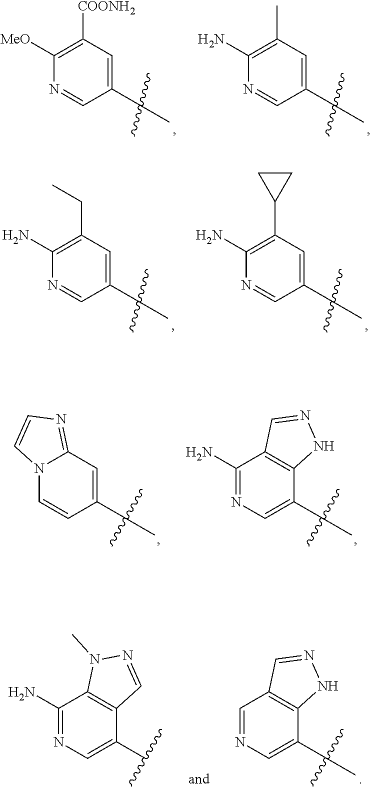

In some embodiments, Ring A is selected from the group consisting of:

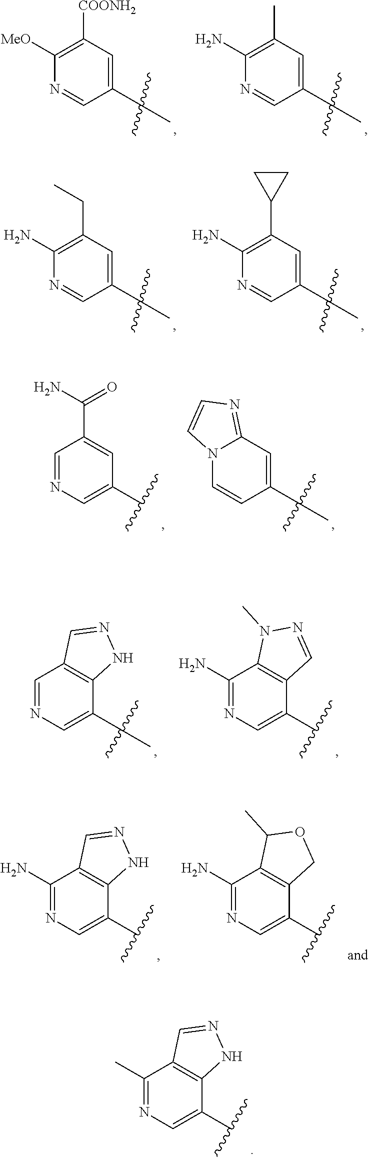

In some embodiments, Ring A is selected from the group consisting of:

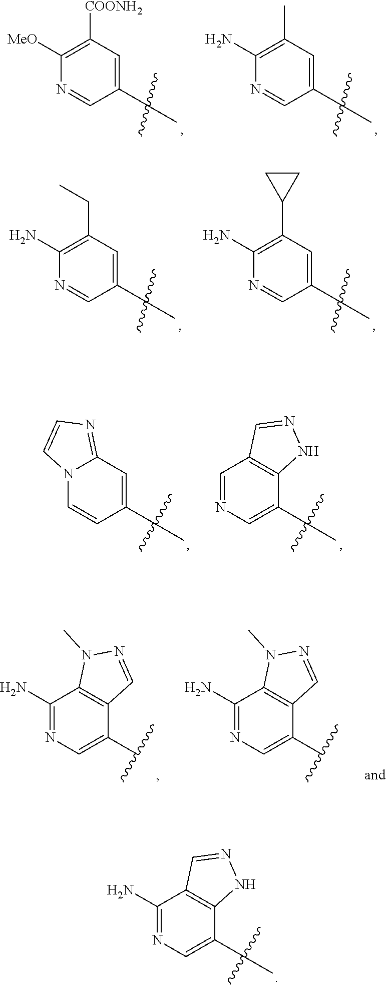

In certain embodiments, Ring A is selected from the group consisting of:

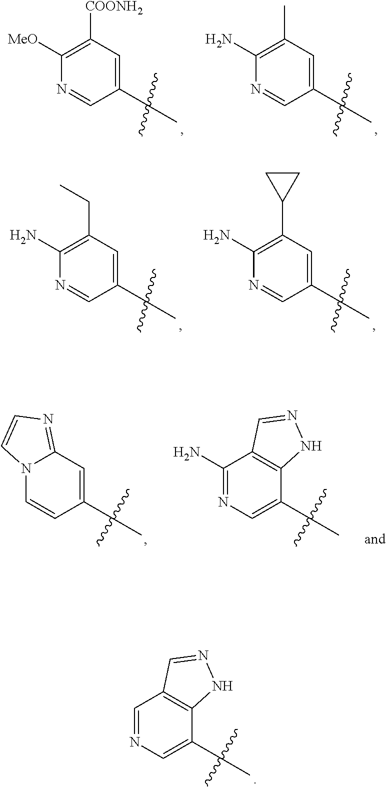

In some embodiments, Ring A is

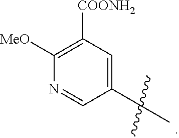

In some embodiments, Ring A is

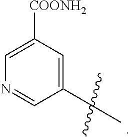

In some embodiments, Ring A is

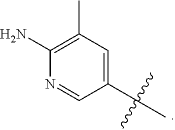 [SMILES: *c1cnc(N)c(C)c1]

In some embodiments, Ring A is

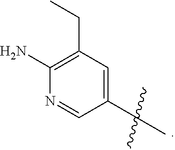 [SMILES: *c1cnc(N)c(CC)c1]

In some embodiments, Ring A is

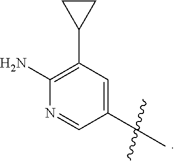 [SMILES: *c1cnc(N)c(C2CC2)c1]

In some embodiments, Ring A is

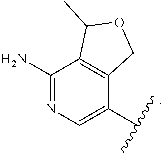 [SMILES: *c1cnc(N)c2c1COC2C]

In some embodiments, Ring A is

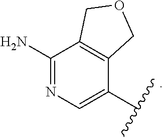 [SMILES: *c1cnc(N)c2c1COC2]

In some embodiments, Ring A is

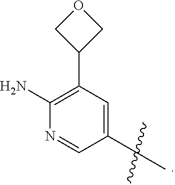 [SMILES: *c1cnc(N)c(C2COC2)c1]

In some embodiments, Ring A is

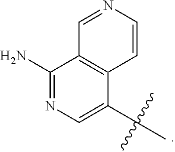 [SMILES: *c1cnc(N)c2cnccc12]

In some embodiments, Ring A is

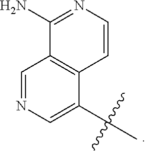 [SMILES: *c1cncc2c(N)nccc12]

In some embodiments, Ring A is

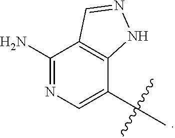 [SMILES: *c1cnc(N)c2cn[nH]c12]

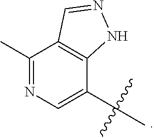 [SMILES: *c1cnc(C)c2cn[nH]c12]

In some embodiments, Ring A is

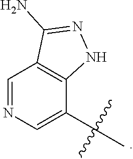 [SMILES: *c1cncc2c(N)n[nH]c12]

In some embodiments, Ring A is

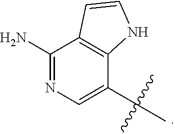 [SMILES: *c1cnc(N)c2cc[nH]c12]

In some embodiments, Ring A is

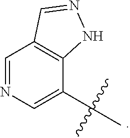 [SMILES: *c1cncc2cn[nH]c12]

In some embodiments, Ring A is

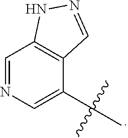 [SMILES: *c1cncc2[nH]ncc12]

In some embodiments, Ring A is.

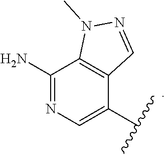 [SMILES: *c1cnc(N)c2c1cnn2C]

In some embodiments, Ring A is

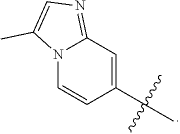 [SMILES: *c1ccn2c(C)cnc2c1]

In some embodiments, Ring A is

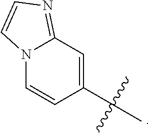 [SMILES: *c1ccn2ccnc2c1]

In some embodiments the compounds of Formula (A) are of Formula (A_1):

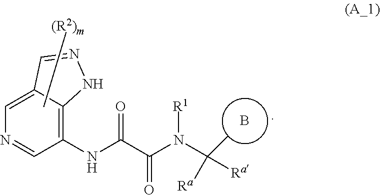

wherein R^{a}, R^{a}′, Ring B, R¹, R² and m are as defined in any of the embodiments described herein.

In some embodiments the compounds of Formula (A) are of Formula (A_1a):

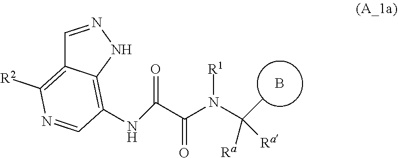

wherein R^{a}, R^{a}′, Ring B, R¹ and R² are as defined in any of the embodiments described herein.

In some embodiments the compounds of Formula (A) are of Formula (A_1b):

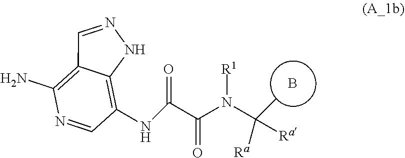

wherein R^{a}, R^{a}′, Ring B and R¹ are as defined in any of the embodiments described herein.

In some embodiments the compounds of Formula (I) are of Formula (A_1c):

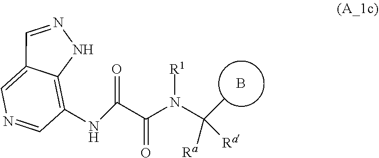

wherein Ring R^{a}, R^{a}′, B and R¹ are as defined in any of the embodiments described herein.

In some embodiments the compounds of Formula (A) are of Formula (A_2):

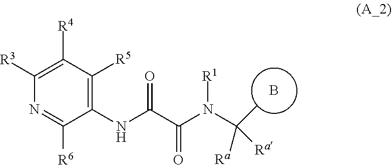

wherein R^{a}, R^{a}′, Ring B, R¹, R³, R⁴, R⁵ and R⁶ are as defined in any of the embodiments described herein.

In some embodiments the compounds of Formula (A) are of Formula (A_2a):

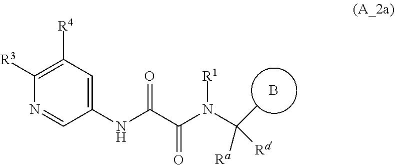

wherein R^{a4}, R^{a}′, Ring B, R¹, R³ and R⁴ are as defined in any of the embodiments described herein.

In some embodiments, the compounds of Formula (A) are of Formula (A_3):

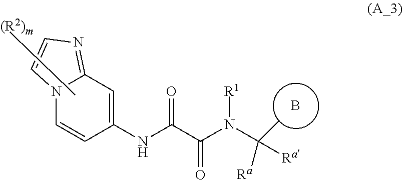

wherein R^{a}, R^{a}′, Ring B, R¹, R², R³, R⁴ and m are as defined in any of the embodiments described herein.

In some embodiments the compounds of Formula (A) are of Formula (A_3a):

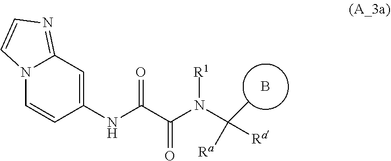

wherein R^{a}, R^{a}′, Ring B and R¹ are as defined in any of the embodiments described herein.

In some embodiments the compounds of Formula (A) are of Formula (A_4):

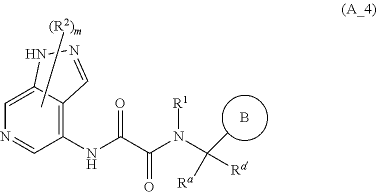

wherein R^{a}, R^{a}′, Ring B, R¹, R² and m are as defined in any of the embodiments described herein.

In some embodiments the compounds of Formula (A) are of Formula (A_4a):

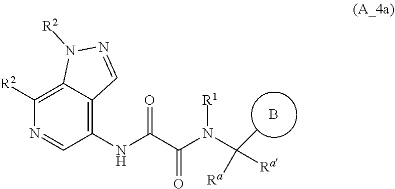

wherein R^{a}, R^{a}′, Ring B, R¹ and R² are as defined in any of the embodiments described herein.

In some embodiments the compounds of Formula (A) are of Formula (A_4b):

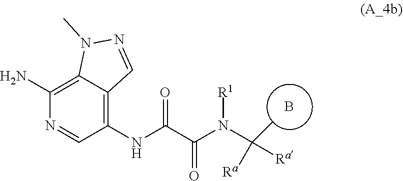

wherein R^{a}, R^{a}′, Ring B and R¹ are as defined in any of the embodiments described herein.

In some embodiments the compounds of Formula (A) are of Formula (A_5):

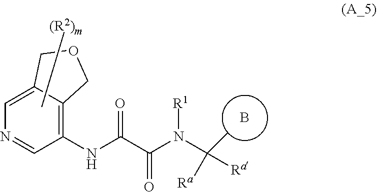

wherein R^{a}, R^{a}′, Ring B, R¹, R² and m are as defined in any of the embodiments described herein.

In some embodiments the compounds of Formula (A) are of Formula (A_5a):

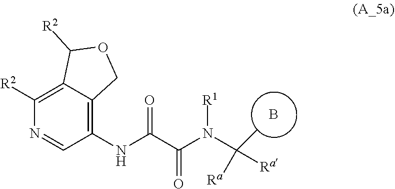

wherein R^{a4}, R^{a}′, Ring B, R¹ and R² are as defined in any of the embodiments described herein.

In some embodiments the compounds of Formula (A) are of Formula (A_5b):

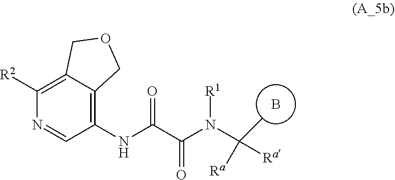

wherein R^{a}, R^{a}′, Ring B, R¹ and R² are as defined in any of the embodiments described herein.

In some embodiments the compounds of Formula (A) are of Formula (A_5c):

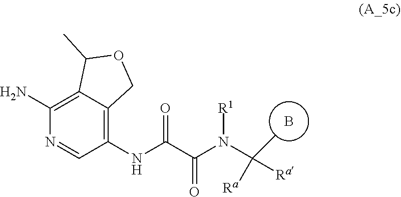

wherein R^{a}, R^{a}′, Ring B and R¹ are as defined in any of the embodiments described herein.

In some embodiments the compounds of Formula (A) are of Formula (A_5d):

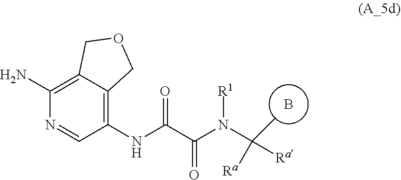

wherein R^{a4}, R^{a}′, Ring B and R¹ are as defined in any of the embodiments described herein.

In some embodiments the compounds of Formula (I) are of Formula (I_1):

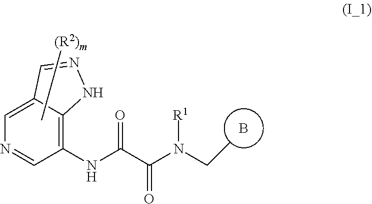

wherein Ring B, R¹, R² and m are as defined in any of the embodiments described herein.

In some embodiments the compounds of Formula (I) are of Formula (I_1a):

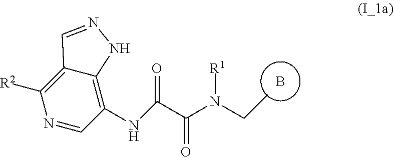

wherein Ring B, R¹ and R² are as defined in any of the embodiments described herein.

In some embodiments the compounds of Formula (I) are of Formula (I_1b):

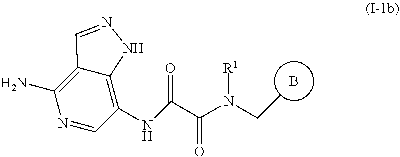

wherein Ring B and R¹ are as defined in any of the embodiments described herein.

In some embodiments the compounds of Formula (I) are of Formula (I_1c):

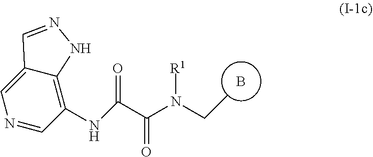

wherein Ring B and R¹ are as defined in any of the embodiments described herein.

In some embodiments the compounds of Formula (I) are of Formula (I_2):

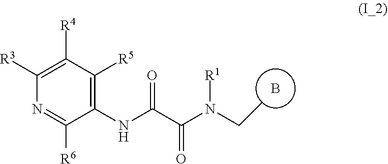

wherein Ring B, R¹, R³, R₄, R⁵ and R⁶ are as defined in any of the embodiments described herein.

In some embodiments the compounds of Formula (I) are of Formula (I_2a):

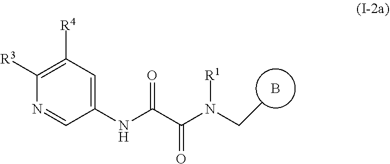

wherein Ring B, R¹, R³ and R⁴ are as defined in any of the embodiments described herein.

In some embodiments, the compounds are of Formula (I_3):

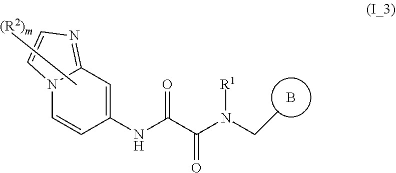

wherein Ring B, R¹, R², R³, R⁴ and m are as defined in any of the embodiments described herein.

In some embodiments the compounds of Formula (I) are of Formula (I_3a):

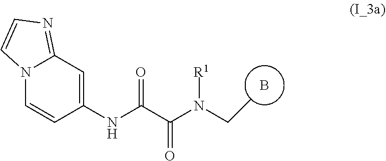

wherein Ring B and R¹ are as defined in any of the embodiments described herein.

In some embodiments the compounds of Formula (I) are of Formula (I_4):

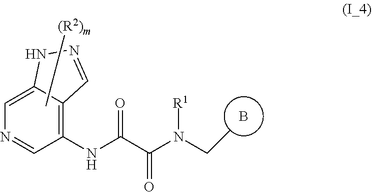

wherein Ring B, R¹, R² and m are as defined in any of the embodiments described herein.

In some embodiments the compounds of Formula (I) are of Formula (I_4a):

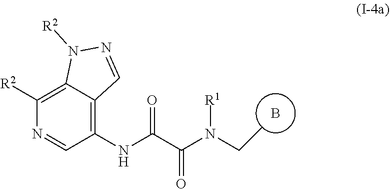

wherein Ring B, R¹ and R² are as defined in any of the embodiments described herein.

In some embodiments the compounds of Formula (I) are of Formula I_4b):

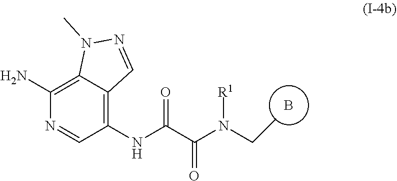

wherein Ring B and R^{I} are as defined in any of the embodiments described herein.

In some embodiments the compounds of Formula (I) are of Formula (I_5):

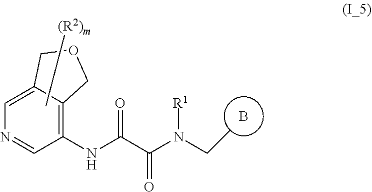

wherein Ring B, R¹, R² and m are as defined in any of the embodiments described herein.

In some embodiments the compounds of Formula (I) are of Formula (I_5a):

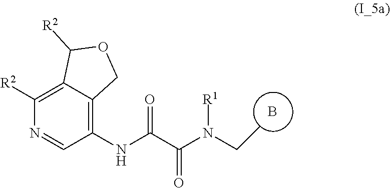

wherein Ring B, R¹ and R² are as defined in any of the embodiments described herein.

In some embodiments the compounds of Formula (I) are of Formula I_5b):

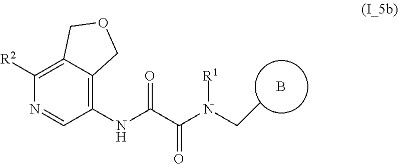

wherein Ring B, R¹ and R² are as defined in any of the embodiments described herein.

In some embodiments the compounds of Formula (I) are of Formula I_5c):

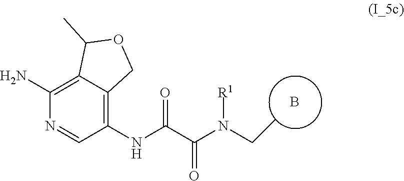

wherein Ring B and R^{I} are as defined in any of the embodiments described herein.

In some embodiments the compounds of Formula (I) are of Formula I_5d):

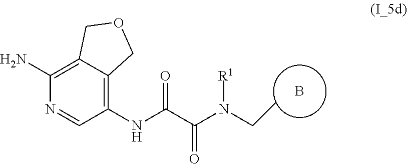

wherein Ring B and R¹ are as defined in any of the embodiments described herein.

As generally defined herein, each R¹ is independently selected from the group consisting of —C₁-C₆ alkyl, —C₂-C₆ heteroalkyl, —C₂-C₆ haloalkyl, —C₃-C₁₀ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position.

In some embodiments, each R¹ is independently selected from the group consisting of —C₁-C₆ alkyl, —C₂-C₆ heteroalkyl, —C₂-C₆ haloalkyl, —C₃-C₁₀ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each substituted at any available position with 0, 1, 2 or 3 instances of R8, wherein each R⁸ is as defined in any of the embodiments described herein.

In some embodiments, each R¹ is independently selected from the group consisting of —C₂-C₆ alkyl, —C₂-C₆ heteroalkyl, —C₂-C₆ haloalkyl, —C₃-C₉ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position (e.g., substituted with 0, 1, 2 or 3 instances of R8, wherein each R⁸ is as defined in any of the embodiments described herein).

In some embodiments, each R¹ is independently selected from the group consisting of —C₁-C₆ alkyl (e.g., -Me, -Et, —Pr, —Pr, -sec-Bu, -^{t}Bu, —CH(CH₃)CH(CH₃)₂, —CH₂CH(CH₃)₂, —CH₂CH(CH₃)CH₂CH₃), —C₂-C₆ heteroalkyl (e.g., —CH₂CH₂OCH₃), —C₂-C₆ haloalkyl (e.g., —CH₂CH₂CF₃), —C₃-C₁₀ carbocyclyl (e.g., cyclopropyl, cyclobutyl, cyclopentyl, cyclohexyl, 2,3-dihydro-1H-indenyl, 1,2,3,4 tetrahydronaphthalenyl), 3-10 membered heterocyclyl (e.g., chromanyl), heteroarylalkyl (e.g., —CH₂-pyridinyl, —CH(CH₃)-pyridinyl-CH₂-pyrimidinyl, —CH(CH₃)-pyrimidinyl, —CH₂-pyrazolyl), arylalkyl (e.g., benzyl, —CH(CH₃)phenyl, —CH₂-naphthalenyl, —CH₂-chromanyl, —CH₂CH₂-phenyl), heterocyclylalkyl (e.g., CH₂-tetrahydropyranyl) and cycloalkylalkyl (e.g., —CH₂-cyclopropyl, —CH₂-cyclobutyl, —CH₂-cyclopentyl, —CH₂-cyclohexyl, —CH(CH₃)cyclopropyl, —CH₂CH₂-cyclopropyl), each substituted at any available position with 0, 1, 2 or 3 instances of R⁸ wherein each R⁸ is as defined in any of the embodiments described herein.

In some embodiments, R¹ is selected from the group consisting of —C₂-C₆ alkyl (e.g., -Et, —Pr, -ⁱPr, -sec-Bu, -^{t}Bu, —CH(CH₃)CH(CH₃)₂), —C₂-C₆ heteroalkyl (e.g., —CH₂CH₂OCH₃), —C₃-C₉ carbocyclyl (e.g., cyclopropyl, cyclobutyl, cyclopentyl, cyclohexyl, 2,3-dihydro-1H-indenyl, 1,2,3,4 tetrahydronaphthalenyl), heteroarylalkyl (e.g., —CH₂-pyridinyl, —CH(CH₃)-pyridinyl-CH₂-pyrimidinyl, —CH(CH₃)-pyrimidinyl), arylalkyl (e.g., benzyl, —CH(CH₃)phenyl, —CH₂CH₂-phenyl), and cycloalkylalkyl (e.g., —CH₂-cyclopropyl, —CH₂-cyclobutyl, —CH₂-cyclopentyl, —CH₂-cyclohexyl, —CH(CH₃)cyclopropyl, —CH₂CH₂-cyclopropyl), each substituted at any available position with 0, 1, 2 or 3 instances of R8, wherein each R⁸ is as defined in any of the embodiments described herein.

In some embodiments, R¹ is independently selected from the group consisting of —C₂-C₆ alkyl (e.g., -Me, -Et, —Pr, -ⁱPr, -sec-Bu, -^{t}Bu, —CH(CH₃)CH(CH₃)₂, —CH₂CH(CH₃)₂, —CH₂CH(CH₃)CH₂CH₃), —C₂-C₆ heteroalkyl (e.g., —CH₂CH₂OCH₃), —C₃-C₉ carbocyclyl (e.g., cyclopropyl, cyclobutyl, cyclopentyl, cyclohexyl, 2,3-dihydro-1H-indenyl, 1,2,3,4 tetrahydronaphthalenyl), heteroarylalkyl (e.g., —CH₂-pyridinyl, —CH(CH₃)-pyridinyl-CH₂-pyrimidinyl, —CH(CH₃)-pyrimidinyl), arylalkyl (e.g., benzyl, —CH(CH₃)phenyl, —CH₂CH₂-phenyl), and cycloalkylalkyl (e.g., —CH₂-cyclopropyl, —CH₂-cyclobutyl, —CH₂-cyclopentyl, —CH₂-cyclohexyl, —CH(CH₃)cyclopropyl, —CH₂CH₂-cyclopropyl), each substituted at any available position with 0, 1, 2 or 3 instances of R8, wherein each R⁸ is as defined in any of the embodiments described herein.

In some embodiments, each R¹ is independently selected from the group consisting of —C₁-C₆ alkyl (e.g., -Me, -Et, —Pr, -ⁱPr, -sec-Bu, -^{t}Bu, —CH(CH₃)CH(CH₃)₂, —CH₂CH(CH₃)₂, —CH₂CH(CH₃)CH₂CH₃), —C₂-C₆ heteroalkyl (e.g., —CH₂CH₂OCH₃) and arylalkyl (e.g., benzyl, —CH(CH₃)phenyl, —CH₂-naphthalenyl, —CH₂-chromanyl), each substituted at any available position with 0, 1 or 2 instances of R⁸ wherein each R⁸ is as defined in any of the embodiments described herein.

In some embodiments, each R¹ is independently selected from the group consisting of —C₁-C₆ alkyl (e.g., -Me, -Et, —Pr, -ⁱPr, -sec-Bu, -^{t}Bu, —CH(CH₃)CH(CH₃)₂, -CH₂CH(CH₃)₂, —CH₂CH(CH₃)CH₂CH₃), —C₂-C₆ heteroalkyl (e.g., —CH₂CH₂OCH₃) and arylalkyl (e.g., benzyl, —CH(CH₃)phenyl, —CH₂-naphthalenyl, —CH₂-chromanyl) wherein the alkyl and the arylalkyl are not further substituted.

In some embodiments, each R¹ is independently selected from the group consisting of -Me, -Et, —Pr, -ⁱPr, -sec-Bu, -^{t}Bu, —CH(CH₃)CH(CH₃)₂, —CH₂CH(CH₃)₂, —CH₂CH(CH₃)CH₂CH₃, —CH₂CH₂OCH₃, —CH₂CH₂CF₃, cyclopropyl, cyclobutyl, cyclopentyl, cyclohexyl, 2,3-dihydro-1H-indenyl, 1,2,3,4 tetrahydronaphthalenyl, chromanyl, —CH₂-pyridinyl, —CH(CH₃)-pyridinyl-CH₂-pyrimidinyl, —CH(CH₃)-pyrimidinyl, —CH₂-pyrazolyl, benzyl, —CH(CH₃)phenyl, —CH₂CH₂-phenyl, CH₂-naphthyl, —CH₂-chromanyl, —CH₂-tetrahydropyranyl, —CH₂-cyclopropyl, —CH₂-cyclobutyl, —CH₂-cyclopentyl, —CH₂-cyclohexyl, —CH(CH₃)cyclopropyl, and —CH₂CH₂-cyclopropyl, each substituted at any available position with 0, 1, 2 or 3 instances of R⁸ wherein each R⁸ is as defined in any of the embodiments described herein.

In some embodiments, R¹ is independently selected from the group consisting of -Me,-Et, —Pr, —Pr, -sec-Bu, -^{t}Bu, —CH(CH₃)CH(CH₃)₂, —CH₂CH(CH₃)₂, —CH₂CH(CH₃)CH₂CH₃, —CH₂CH₂OCH₃, cyclopropyl, cyclobutyl, cyclopentyl, cyclohexyl, 2,3-dihydro-1H-indenyl, 1,2,3,4 tetrahydronaphthalenyl, —CH₂-pyridinyl, —CH(CH₃)-pyridinyl-CH₂-pyrimidinyl, —CH(CH₃)-pyrimidinyl, benzyl, —CH(CH₃)phenyl, —CH₂CH₂-phenyl, —CH₂-cyclopropyl, —CH₂-cyclobutyl, —CH₂-cyclopentyl, —CH₂-cyclohexyl, —CH(CH₃)cyclopropyl, and —CH₂CH₂-cyclopropyl, each substituted at any available position with 0, 1, 2 or 3 instances of R⁸ wherein each R⁸ is as defined in any of the embodiments described herein.

In some embodiments, R¹ is independently selected from the group consisting of -Et, —Pr, -ⁱPr, -sec-Bu, -^{t}Bu, —CH(CH₃)CH(CH₃)₂, —CH₂CH₂OCH₃, cyclopropyl, cyclobutyl, cyclopentyl, cyclohexyl, 2,3-dihydro-1H-indenyl, 1,2,3,4 tetrahydronaphthalenyl, —CH₂-pyridinyl, —CH(CH₃)-pyridinyl-CH₂-pyrimidinyl, —CH(CH₃)-pyrimidinyl, benzyl, —CH(CH₃)phenyl, —CH₂CH₂-phenyl, —CH₂-cyclopropyl, —CH₂-cyclobutyl, —CH₂-cyclopentyl, —CH₂-cyclohexyl, —CH(CH₃)cyclopropyl, and —CH₂CH₂-cyclopropyl, each substituted at any available position with 0, 1, 2 or 3 instances of R8, wherein each R⁸ is as defined in any of the embodiments described herein.

In some embodiments, each R¹ is independently selected from the group consisting of -Me, -Et, —Pr, —CH(CH₃)CH(CH₃)₂, —CH₂CH(CH₃)₂, —CH₂CH(CH₃)CH₂CH₃, —CH₂CH₂OCH₃, —CH₂CH₂CF₃, cyclopropyl, cyclobutyl, cyclopentyl, 2,3-dihydro-1H-indenyl, 1,2,3,4 tetrahydronaphthalenyl, chromanyl, —CH₂-cyclopropyl, —CH₂-cyclohexyl, —CH₂CH₂-cyclopropyl, —CH₂-tetrahydropyranyl, —CH₂-pyridinyl, —CH₂-pyrimidinyl, —CH₂-pyrazolyl, -benzyl CH₂-chromanyl, CH₂-naphthyl, and —CH₂-cyclopropyl, each substituted at any available position with 0, 1 or 2 instances of R⁸ wherein each wherein R⁸ is as defined in any of the embodiments described herein.

In some embodiments, R¹ is independently selected from the group consisting of -Me,-Et, —Pr, —Pr, —CH(CH₃)CH(CH₃)₂, —CH₂CH(CH₃)₂, —CH₂CH(CH₃)CH₂CH₃, —CH₂CH₂OCH₃, cyclopentyl, 2,3-dihydro-1H-inden-1-yl, 1,2,3,4 tetrahydronaphthalen-1-yl, —CH₂-pyrimidinyl, —CH(CH₃)-pyrimidinyl, benzyl and —CH₂-cyclopropyl, each substituted at any available position with 0 or 1 instances of R⁸ wherein each R⁸ is as defined in any of the embodiments described herein.

In some embodiments, each R¹ is independently selected from the group consisting of -Et, —Pr, —CH(CH₃)CH(CH₃)₂, —CH₂CH₂OCH₃, cyclopentyl, 2,3-dihydro-1H-inden-1-yl, 1,2,3,4 tetrahydronaphthalen-1-yl, —CH₂-pyrimidinyl, —CH(CH₃)-pyrimidinyl, benzyl and —CH₂-cyclopropyl, each substituted at any available position with 0 or 1 instances of R⁸ wherein each R⁸ is independently selected from the group consisting of —F, -Me, —OCHF₂, -cyclopropyl, and —CH₂OCH₃.

In some embodiments, each R¹ is independently selected from the group consisting of -Me, -Et, —Pr, —CH(CH₃)CH(CH₃)₂, CH₂CH(CH₃)₂, —CH₂CH(CH₃)CH₂CH₃, —CH₂CH₂CF₃, —CH₂CH₂OCH₃,

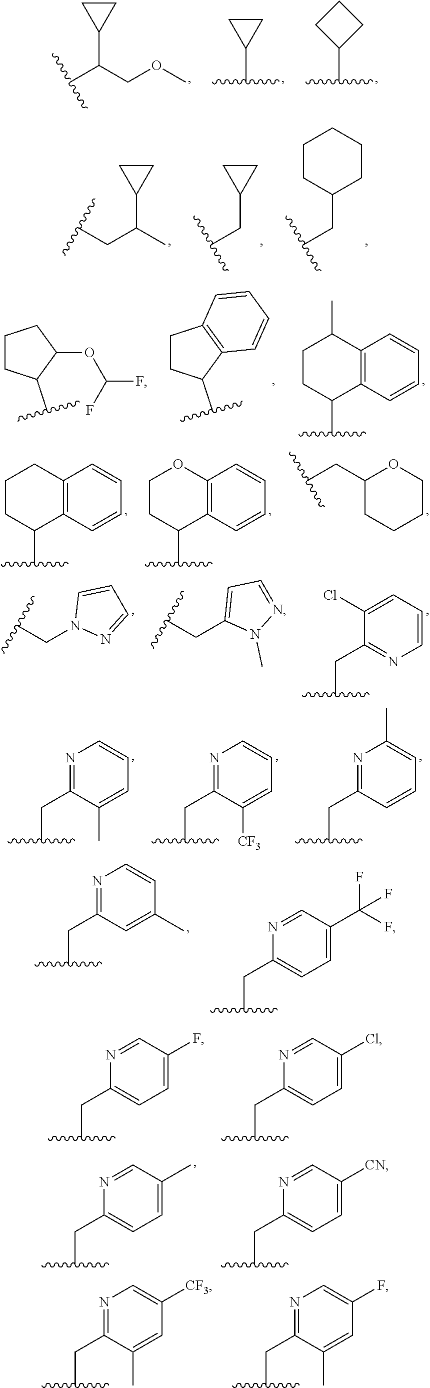

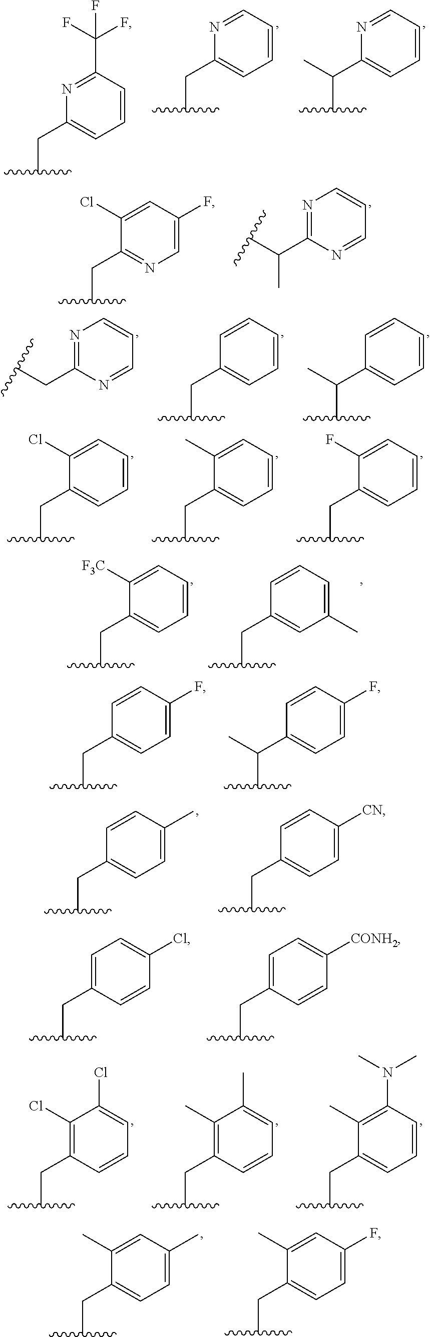

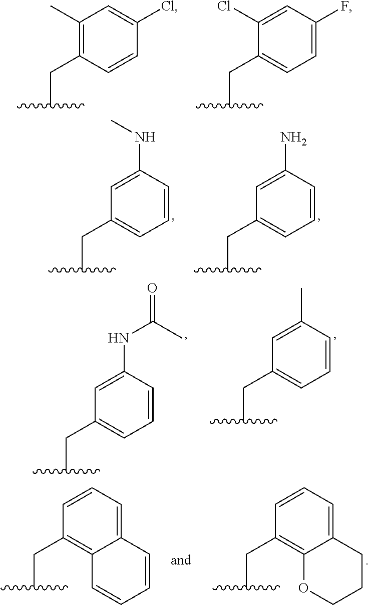

In some embodiments, R¹ is independently selected from the group consisting of -Me,-Et, —Pr, —CH(CH₃)CH(CH₃)₂, —CH₂CH(CH₃)₂, —CH₂CH(CH₃)CH₂CH₃, —CH₂CH₂OCH₃,

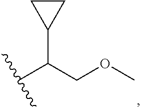 [SMILES: *C(COC)C1CC1]

cyclobutyl,

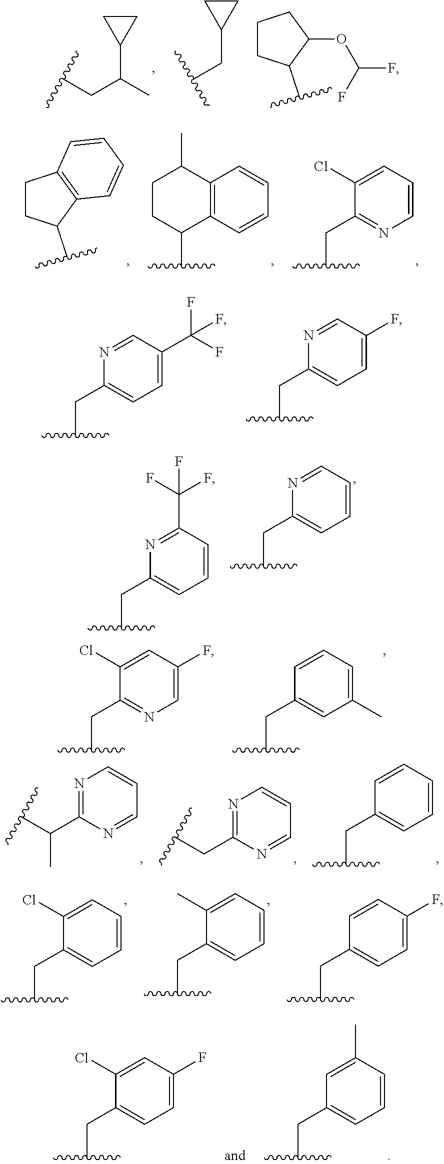

In some embodiments, R¹ is independently selected from the group consisting of -Me,-Et, —Pr, —CH(CH₃)CH(CH₃)₂, —CH₂CH(CH₃)₂, —CH₂CH(CH₃)CH₂CH₃, -CH₂CH₂OCH₃

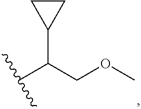 [SMILES: *C(COC)C1CC1]

cyclobutyl,

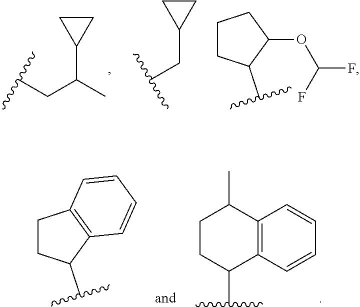

In some embodiments, R¹ is selected from the group consisting of -Et, —Pr, —CH(CH₃)CH(CH₃)₂, —CH₂CH₂OCH₃, cyclopentyl, 2,3-dihydro-1H-inden-1-yl, 1,2,3,4 tetrahydronaphthalen-1-yl, —CH₂-pyrimidinyl, —CH(CH₃)-pyrimidinyl, benzyl and —CH₂-cyclopropyl, each substituted at any available position with 0 or 1 instances of R8, wherein R⁸ is as defined in any of the embodiments described herein. In some embodiments, R⁸ is selected from the group consisting of —F, -Me, —OCHF₂, -cyclopropyl, and —CH₂OCH₃;

In some embodiments, each R¹ is independently selected from the group consisting of -Me, -Et, —Pr, —CH(CH₃)CH(CH₃)₂, —CH₂CH(CH₃)₂, —CH₂CH(CH₃)CH₂CH₃, —CH₂CH₂OCH₃, —CH₂CH₂CF₃, cyclopropyl, cyclobutyl, cyclopentyl, 2,3-dihydro-1H-indenyl, 1,2,3,4 tetrahydronaphthalenyl, chromanyl, —CH₂-cyclopropyl, —CH₂-cyclohexyl, —CH(CH₃)cyclopropyl, and —CH₂CH₂-cyclopropyl, each substituted at any available position with 0 or 1 instances of R⁸ wherein each R⁸ is independently selected from the group consisting of -Me and —OCHF₂.

In some embodiments, each R¹ is independently selected from the group consisting of -Me, -Et, —Pr, —CH(CH₃)CH(CH₃)₂, CH₂CH(CH₃)₂, —CH₂CH(CH₃)CH₂CH₃, -CH₂CH₂CF₃, —CH₂CH₂OCH₃,

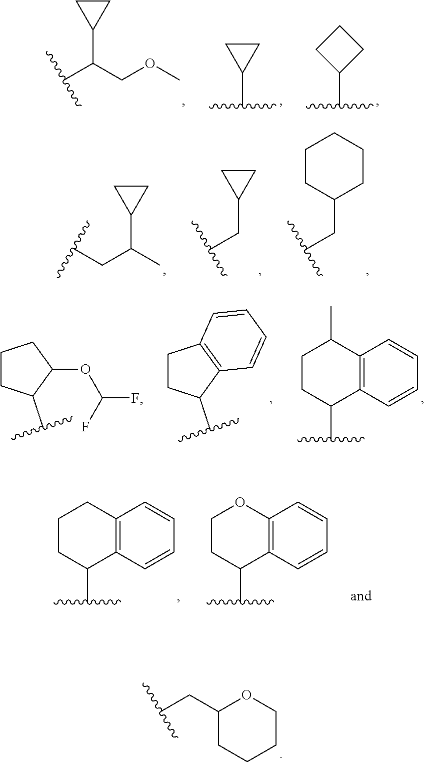

In some embodiments, each R′ is independently selected from the group consisting of -Me, -Et, —Pr, —CH(CH₃)CH(CH₃)₂, —CH₂CH(CH₃)₂ and —CH₂CH(CH₃)CH₂CH₃.

In some embodiments, each R¹ is independently selected from the group consisting of -Me, -Et, benzyl, —CH₂-pyridinyl and CH₂-pyrimidinyl, wherein the benzyl, —CH₂-pyridinyl and CH₂-pyrimidinyl are substituted at any available positions with 0, 1 or 2 substituents independently selected from -Me, —F, —Cl and —CF₃.

In some embodiments, each R¹ is independently selected from the group consisting of benzyl, —CH₂-pyridinyl and CH₂-pyrimidinyl, wherein the benzyl, —CH₂-pyridinyl and —CH₂-pyrimidinyl are substituted at any available positions with 0, 1 or 2 substituents independently selected from -Me, —F, —Cl and —CF₃.

In some embodiments, each R¹ is independently selected from the group consisting of -Me, -Et and benzyl wherein the benzyl is substituted at any available positions with 0, 1 or 2 substituents independently selected from Me, —F, —Cl and —CF₃.

In some embodiments, each R¹ is independently selected from the group consisting of -Me, -Et, —CH₂-phenyl and —CH(CH₃)phenyl, wherein the phenyl is substituted at any available positions with 0, 1 or 2 substituents independently selected from Me, —F, —Cl and —CF₃.

In some embodiments, R¹ is —C₂-C₆ alkyl (e.g., -Et, —Pr, —Pr, -Bu, -sec-Bu, -iso-Bu,-^{t}Bu, -Pentyl, -iso-Pentyl, -neo-Pentyl, —CH(CH₃)CH(CH₃)₂), substituted with 0, 1, 2 or 3 instances of R⁸ (i.e., wherein one or more hydrogens of the alkyl group is replaced with R⁸), wherein R⁸ is as defined in any of the embodiments described herein. In some embodiments, each R′ is independently selected from the group consisting of -Me, -Et, —Pr, —CH(CH₃)CH(CH₃)₂, —CH₂CH(CH₃)₂, —CH₂CH(CH₃)CH₂CH₃ and

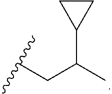 [SMILES: *CC(C)C1CC1]

In some embodiments, each R¹ is independently selected from the group consisting of -Me and -Et, In some embodiments, R¹ is -Me. In some embodiments, R¹ is -Et. In some embodiments, R¹ is —Pr. In some embodiments, R¹ is -ⁱPr. In some embodiments, R¹ is -Bu. In some embodiments, R¹ is -sec-Bu. In some embodiments, R¹ is -iso-Bu. In some embodiments, R¹ is -^{t}Bu. In some embodiments, R¹ is -Pentyl. In some embodiments, R¹ is -iso-Pentyl. In some embodiments, R¹ is -neo-Pentyl. In some embodiments, R¹ is —CH(CH₃)CH(CH₃)₂. In some embodiments, R¹ is —CH₂CH(CH₃)CH₂CH₃. In some embodiments, R¹ is —CH₂CH(CH₃)₂. In some embodiments, the —C₂-C₆ alkyl is unsubstituted. In some embodiments, the —C₂-C₆ alkyl is substituted with 1 instance of R⁸. In some embodiments, the —C₂-C₆ alkyl is substituted with 2 instances of R⁸. In some embodiments, the —C₂-C₆ alkyl is substituted with 3 instances of R⁸. In some embodiments, R′ is —CH₂CH(CH₃)₂ substituted with cyclopropyl. In some embodiments, R′ is

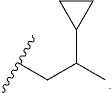 [SMILES: *CC(C)C1CC1]

In some embodiments, R¹ is —C₂-C₆ heteroalkyl substituted with 0, 1, 2 or 3 instances of R⁸ (i.e., wherein one or more hydrogens of the alkyl group is replaced with R⁸), wherein R⁸ is as defined in any of the embodiments described herein. In some embodiments, R¹ is alkoxymethyl (e.g., —CH₂OCH₃, —CH₂CH₂OCH₃). In some embodiments, R¹ is methoxymethyl (—CH₂OCH₃). In some embodiments, R¹ is —CH₂CH₂OCH₃. In some embodiments, R¹ is aminomethyl (e.g., —CH₂NHCH₃, —CH₂N(CH₃)₂). In some embodiments, the —C₂-C₆ heteroalkyl is substituted with 1 instance of R⁸. In some embodiments, the —C₂-C₆ heteroalkyl is substituted with 2 instances of R⁸. In some embodiments, the —C₂-C₆ heteroalkyl is substituted with 3 instances of R⁸. In some embodiments, R¹ is —CH₂CH₂OCH₃ substituted with cyclopropyl. In some embodiments, R¹ is

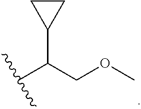 [SMILES: *C(COC)C1CC1]

In some embodiments, R¹ is —C₂-C₆ haloalkyl (e.g., —CH₂CF₃, —CF₂CH₃, —CH₂CHF2, —CH₂CH₂CF₃) substituted with 0, 1, 2 or 3 instances of R⁸ (i.e., wherein one or more hydrogens of the alkyl group is replaced with R⁸), wherein R⁸ is as defined in any of the embodiments described herein. In some embodiments, R¹ is —CH₂CH₂CF₃.

In some embodiments, R¹ is C₃-C₁₀ carbocyclyl (e.g., cyclopropyl, cyclobutyl, cyclopentyl, cyclohexyl, 2,3-dihydro-1H-indenyl, 1,2,3,4 tetrahydronaphthalenyl) substituted with 0, 1, 2 or 3 instances of R⁸ wherein R⁸ is as defined in any of the embodiments described herein. In some embodiments, R¹ is selected from the group consisting of C₃-C₇ monocyclic cycloalkyl (e.g., cyclopropyl, cyclobutyl, cyclopentyl, cyclohexyl, cycloheptyl), C₄-C₁₀ bicyclic cycloalkyl, C₅-C₇ monocyclic cycloalkenyl, C₆-C₁₀ bicyclic cycloalkenyl and C₄-C₆ cycloalkenyl fused with a phenyl ring to form a C₈-C₁₀ partially aromatic carbocyclyl (e.g., 2,3-dihydro-1H-indenyl, 1,2,3,4 tetrahydronaphthalenyl).

In some embodiments, R¹ is selected from the group consisting of C₃-C₇ monocyclic cycloalkyl (e.g., cyclopropyl, cyclobutyl, cyclopentyl, cyclohexyl, cycloheptyl) and C₄-C₆ cycloalkenyl fused with a phenyl ring to form a C5-C₁₀ partially aromatic carbocyclyl (e.g., 2,3-dihydro-1H-indenyl, 1,2,3,4 tetrahydronaphthalenyl).

In some embodiments, R¹ is selected from the group consisting of cyclopropyl, cyclobutyl, cyclopentyl, cyclohexyl, 2,3-dihydro-1H-indenyl (e.g., 2,3-dihydro-1H-inden-1-yl) and 1,2,3,4 tetrahydronaphthalenyl (e.g., 1,2,3,4 tetrahydronaphthalen-1-yl). In some embodiments, R′ is selected from the group consisting of cyclopropyl, cyclobutyl, cyclopentyl, 2,3-dihydro-1H-indenyl (e.g., 2,3-dihydro-1H-inden-1-yl) and 1,2,3,4 tetrahydronaphthalenyl (e.g., 1,2,3,4 tetrahydronaphthalen-1-yl). In some embodiments, R¹ is selected from the group consisting of cyclopentyl, 2,3-dihydro-1H-indenyl (e.g., 2,3-dihydro-1H-inden-1-yl) and 1,2,3,4 tetrahydronaphthalenyl (e.g., 1,2,3,4 tetrahydronaphthalen-1-yl). In some embodiments, R′ is cyclopropyl. In some embodiments R¹ is cyclobutyl. In some embodiments, R¹ is cyclopentyl. In some embodiments, R¹ is cyclohexyl. In some embodiments, R¹ is 2,3-dihydro-1H-indenyl (e.g., 2,3-dihydro-1H-inden-1-yl). In some embodiments, R′ is 1,2,3,4 tetrahydronaphthalenyl (e.g., 1,2,3,4 tetrahydronaphthalen-1-yl). In some embodiments, the C₃-C₁₀ carbocyclyl is unsubstituted. In some embodiments, the C₃-C₁₀ carbocyclyl is substituted with 1 instance of R⁸. In some embodiments, the C₃-C₁₀ carbocyclyl is substituted with 2 instances of R⁸. In some embodiments, the C3-C₁₀ carbocyclyl is substituted with 3 instances of R⁸. In some embodiments, R¹ is selected from the group consisting of 2,3-dihydro-1H-inden-1-yl, 4-methyl-1,2,3,4-tetrahydronaphthalen-1-yl and 2-(difluoromethoxy)cyclopentyl. In some embodiments, R¹ is In some

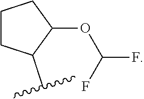 [SMILES: *C1CCCC1OC(F)F]

In some embodiments, R¹ is

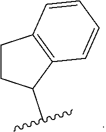 [SMILES: *C1CCc2ccccc21]

In some embodiments, R¹ is

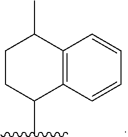 [SMILES: *C1CCC(C)c2ccccc21]

In some embodiments, R¹ is

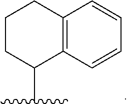 [SMILES: *C1CCCc2ccccc21]

In some embodiments, R¹ is 3-10 membered heterocyclyl substituted with 0, 1, 2 or 3 instances of R⁸ wherein R⁸ is as defined in any of the embodiments described herein. In some embodiments, R¹ is 3-6 membered heterocyclyl (e.g., oxetanyl, tetrahydropyranyl, tetrahydrofuranyl, azetidinyl, pyrrolidinyl, piperidinyl, piperazinyl, morpholinyl, azepanyl, chromanyl). In some embodiments, R¹ is oxetanyl (e.g., oxetan-3-yl). In some embodiments, R¹ is tetrahydropyranyl. In some embodiments, R¹ is tetrahydrofuranyl. In some embodiments, R¹ is azetidinyl. In some embodiments, R¹ is pyrrolidinyl. In some embodiments, R¹ is piperidinyl. In some embodiments, R¹ is piperazinyl. In some embodiments, R¹ is morpholinyl. In some embodiments, R¹ is azepanyl. In some embodiments, R¹ is chromanyl. In some embodiments, R¹ is

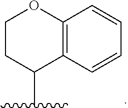 [SMILES: *C1CCOc2ccccc21]

In some embodiments, R¹ is a 5-10 membered heteroaryl (e.g., a 5-6 membered monocyclic heteroaryl or an 8-10 membered bicyclic heteroaryl containing 1-3 heteroatoms selected from the group consisting of N, O and S), substituted with 0, 1, 2 or 3 instances of R⁸ wherein R⁸ is as defined in any of the embodiments described herein. In some embodiments, R¹ is a 5-6 membered monocyclic heteroaryl (e.g., a 5-membered monocyclic heteroaryl containing 1-3 heteroatoms selected from the group consisting of O, N and S, a 6-membered monocyclic heteroaryl containing 1-3 N heteroatoms). In some embodiments, R¹ is a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, R¹ is a 6-membered monocyclic heteroaryl (e.g., pyridyl, pyrimidinyl, triazinyl, pyrazinyl, pyridazinyl). In some embodiments, the heteroaryl is substituted with 0, 1, 2 or 3 instances of -Me, —OH, —C(═O)CH₃, —C(═O)NHCH₃, —NH₂, —NHC(═O)CH₃ or a combination thereof.

In some embodiments, R¹ is a 6-10 membered mono or bicyclic aryl substituted with 0, 1, 2 or 3 instances of R⁸ wherein R⁸ is as defined in any of the embodiments described herein. In some embodiments, R¹ is phenyl substituted with 0, 1, 2 or 3 instances of R⁸ wherein R⁸ is as defined in any of the embodiments described herein.

In some embodiments R¹ is cycloalkylalkyl (e.g., cyclopropylmethyl, cyclopropylethyl, cyclopropylpropyl, —CH₂CH(CH₃)cyclopropyl, cyclobutylmethyl, cyclobutylethyl, cyclopentylmethyl, cyclopentylethyl, cyclohexylmethyl, cyclohexylethyl, cycloheptylmethyl, cycloheptylethyl), substituted with 0, 1, 2 or 3 instances of R⁸ wherein R⁸ is as defined in any of the embodiments described herein. In some embodiments, R¹ is selected from the group consisting of

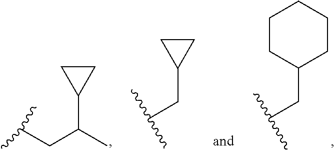

In some embodiments R¹ is cyclopropylmethyl (—CH₂cyclopropyl). In some embodiments R¹ is cyclopropylethyl (—CH₂CH₂cyclopropyl). In some embodiments, R¹ is cyclopropylpropyl (—CH₂CH₂CH₂cyclopropyl). In some embodiments, R¹ is —CH₂CH(CH₃)cyclopropyl. In some embodiments R¹ is cyclobutylmethyl (—CH₂cyclobutyl). In some embodiments R¹ is cyclobutylethyl (—CH₂CH₂cyclobutyl). In some embodiments R¹ is cyclopentylmethyl (—CH₂cyclopentyl). In some embodiments R¹ is cyclopentylethyl (—CH₂CH₂cyclopentyl). In some embodiments R¹ is cyclohexylmethyl (—CH₂cyclohexyl). In some embodiments R¹ is cyclohexylethyl (—CH₂CH₂cyclohexyl). In some embodiments R′ is cycloheptylmethyl (—CH₂cycloheptyl). In some embodiments R′ is cycloheptylethyl (—CH₂CH₂cycloheptyl).

In some embodiments, the cycloalkylalkyl is unsubstituted. In some embodiments, the cycloalkylalkyl is substituted with 1 instance of R⁸. In some embodiments, the cycloalkylalkyl is substituted with 2 instances of R⁸. In some embodiments, the cycloalkylalkyl is substituted with 3 instances of R⁸. In some embodiments, R¹ is -CH₂cyclopropyl substituted with —CH₂OCH₃.

In some embodiments, R¹ is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl) substituted with 0, 1, 2 or 3 instances of R³ wherein R⁸ is as defined in any of the embodiments described herein. In some embodiments, R′ is tetrahydropyranylmethyl. In some embodiments, R¹ is

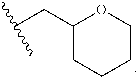 [SMILES: *CC1CCCCO1]

In some embodiments, R¹ is arylalkyl (e.g., benzyl) substituted with 0, 1, 2 or 3 instances of R⁸ wherein R⁸ is as defined in any of the embodiments described herein. In some embodiments, R¹ is benzyl. In some embodiments, the arylalkyl (e.g., benzyl) is unsubstituted. In some embodiments, the arylalkyl (e.g., benzyl) is substituted with 1 instance of R⁸. In some embodiments, the arylalkyl (e.g., benzyl) is substituted with 2 instances of R⁸ In some embodiments, the arylalkyl (e.g., benzyl) is substituted with 3 instances of R⁸. In some embodiments, R¹ is benzyl substituted with 0, 1, 2 or 3 instances of R⁸ wherein R⁸ is as defined in any of the embodiments described herein. In some embodiments, R¹ is benzyl substituted at any available positions with 0, 1 or 2 substituents independently selected from Me, —F, —Cl and —CF₃.

In some embodiments, R^{I} is selected from the group consisting of:

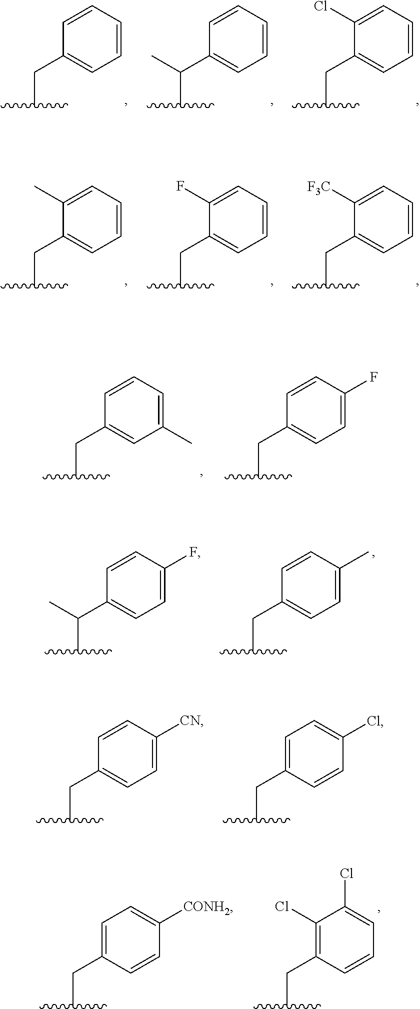

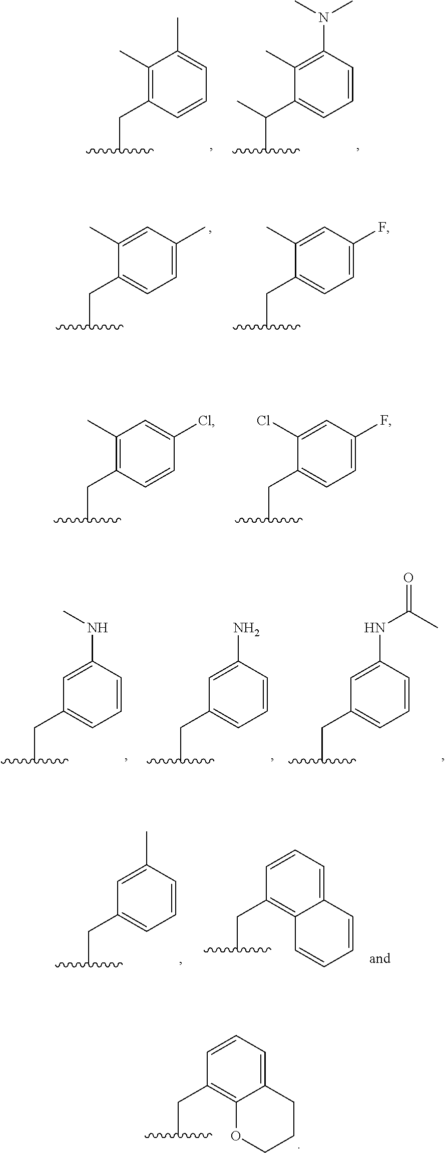

In some embodiments R¹ is selected from

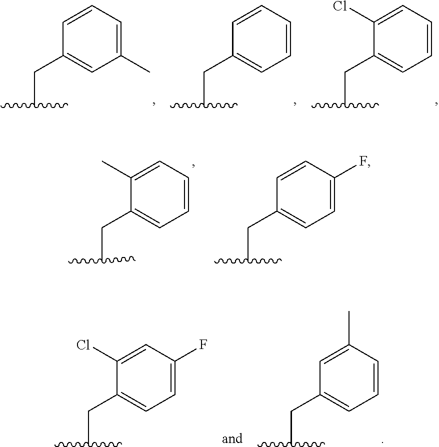

In some embodiments, R¹ is

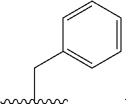 [SMILES: *Cc1ccccc1]

In some embodiments, R¹ is

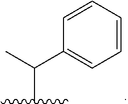 [SMILES: *C(C)c1ccccc1]

In some embodiments, R¹ is

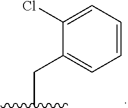 [SMILES: *Cc1ccccc1Cl]

In some embodiments, R¹ is.

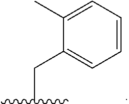 [SMILES: *Cc1ccccc1C]

In some embodiments, R¹ is

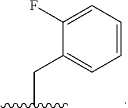 [SMILES: *Cc1ccccc1F]

In some embodiments, R¹ is

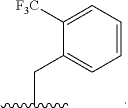 [SMILES: *Cc1ccccc1C(F)(F)F]

In some embodiments, R¹ is

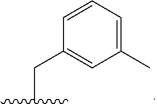 [SMILES: *Cc1cccc(C)c1]

In some embodiments, R¹ is

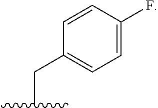 [SMILES: *Cc1ccc(F)cc1]

In some embodiments, R¹ is

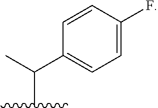 [SMILES: *C(C)c1ccc(F)cc1]

In some embodiments, R¹ is

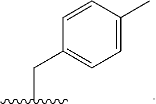 [SMILES: *Cc1ccc(C)cc1]

In some embodiments, R¹ is

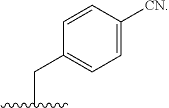 [SMILES: *Cc1ccc(C#N)cc1]

In some embodiments, R¹ is

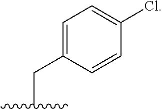 [SMILES: *Cc1ccc(Cl)cc1]

In some embodiments, R¹ is

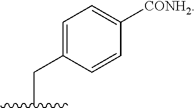 [SMILES: *Cc1ccc(C(N)=O)cc1]

In some embodiments, R¹ is

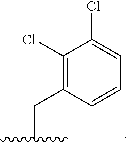 [SMILES: *Cc1cccc(Cl)c1Cl]

In some embodiments, R′ is

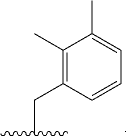 [SMILES: *Cc1cccc(C)c1C]

In some embodiments, R¹ is

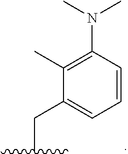 [SMILES: *Cc1cccc(N(C)C)c1C]

In some embodiments, R¹ is

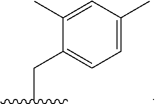 [SMILES: *Cc1ccc(C)cc1C]

In some embodiments, R¹ is

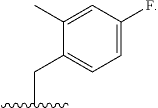 [SMILES: *Cc1ccc(F)cc1C]

In some embodiments, R¹ is

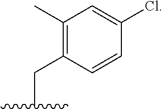 [SMILES: *Cc1ccc(Cl)cc1C]

In some embodiments, R¹ is

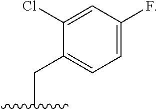 [SMILES: *Cc1ccc(F)cc1Cl]

In some embodiments, R¹ is

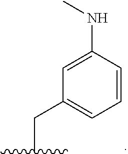 [SMILES: *Cc1cccc(NC)c1]

In some embodiments, R¹ is

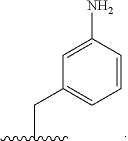 [SMILES: *Cc1cccc(N)c1]

In some embodiments, R¹ is

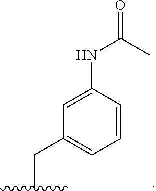 [SMILES: *Cc1cccc(NC(C)=O)c1]

In some embodiments, R¹ is

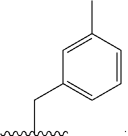 [SMILES: *Cc1cccc(C)c1]

In some embodiments, R¹ is

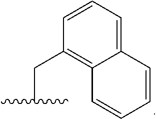 [SMILES: *Cc1cccc2ccccc12]

In some embodiments, R¹ is

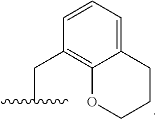 [SMILES: *Cc1cccc2c1OCCC2]

In some embodiments, R¹ is heteroarylalkyl (e.g., pyridinylmethyl, pyridinylethyl, thiazolylmethyl, triazolylmethyl, pyrazolylmethyl, pyrimidinylmethyl, pyrimidinylethyl) substituted with 0, 1, 2 or 3 instances of R⁸ wherein R⁸ is as defined in any of the embodiments described herein. In some embodiments, R¹ is selected from the group consisting of pyridinylmethyl (—CH₂pyridinyl), pyridinylethyl (—CH₂CH₂pyridinyl, —CH(CH₃)pyridinyl), thiazolylmethyl (—CH₂thiazolyl), triazolylethyl (—CH₂CH₂triazolyl), pyrazolylmethyl (—CH₂pyrazolyl), pyrimidinylmethyl (—CH₂pyrimidinyl) and pyrimidinylethyl (—CH₂CH₂pyrimidinyl, —CH(CH₃)pyrimidinyl)) substituted with 0, 1, 2 or 3 instances of R³ wherein R⁸ is as defined in any of the embodiments described herein. In some embodiments, R¹ is pyridinylmethyl (—CH₂pyridinyl) substituted with 0, 1, 2 or 3 instances of R8 wherein R⁸ is as defined in any of the embodiments described herein. In some embodiments, R¹ is pyridinylethyl (—CH₂CH₂pyridinyl, —CH(CH₃)pyridinyl) substituted with 0, 1, 2 or 3 instances of R⁸ wherein R⁸ is as defined in any of the embodiments described herein. In some embodiments, R¹ is thiazolylmethyl (—CH₂thiazolyl) substituted with 0, 1, 2 or 3 instances of R⁸ wherein R⁸ is as defined in any of the embodiments described herein. In some embodiments, R¹ is triazolylethyl (—CH₂CH₂triazolyl) substituted with 0, 1, 2 or 3 instances of R& wherein R⁸ is as defined in any of the embodiments described herein. In some embodiments, R¹ is pyrazolylmethyl (—CH₂pyrazolyl) substituted with 0, 1, 2 or 3 instances of R8 wherein R⁸ is as defined in any of the embodiments described herein. In some embodiments, R¹ is pyrimidinylmethyl (—CH₂pyrimidinyl) substituted with 0, 1, 2 or 3 instances of R⁸ wherein R⁸ is as defined in any of the embodiments described herein. In some embodiments, R¹ is pyrimidinylethyl (—CH₂CH₂pyrimidinyl, —CH(CH₃)pyrimidinyl) substituted with 0, 1, 2 or 3 instances of R⁸ wherein R⁸ is as defined in any of the embodiments described herein.

In some embodiments, each R¹ is —CH₂-pyridinyl substituted at any available positions with 0, 1 or 2 substituents independently selected from -Me, —F, —Cl and —CF₃.

In some embodiments, each R¹ is CH₂-pyrimidinyl substituted at any available positions with 0, 1 or 2 substituents independently selected from -Me, —F, —Cl and —CF₃.

In some embodiments, the heteroarylalkyl is unsubstituted. In some embodiments, the heteroarylalkyl is substituted with 1 instance of R⁸. In some embodiments, the heteroarylalkyl is substituted with 2 instances of R⁸. In some embodiments, the heteroarylalkyl is substituted with 3 instances of R⁸. In some embodiments, R¹ is selected from the group consisting of —CH₂-pyridin-2-yl substituted with one or two substituents independently selected from —F, —Cl and —CF₃, —CH₂-pyrimidin-2-yl and —CH(CH₃)-pyrimidin-2-yl. In some embodiments, R¹ is selected from the group consisting of —CH₂-pyrimidin-2-yl and —CH(CH₃)-pyrimidin-2-yl.

In some embodiments, R^{I} is selected from a group consisting of:

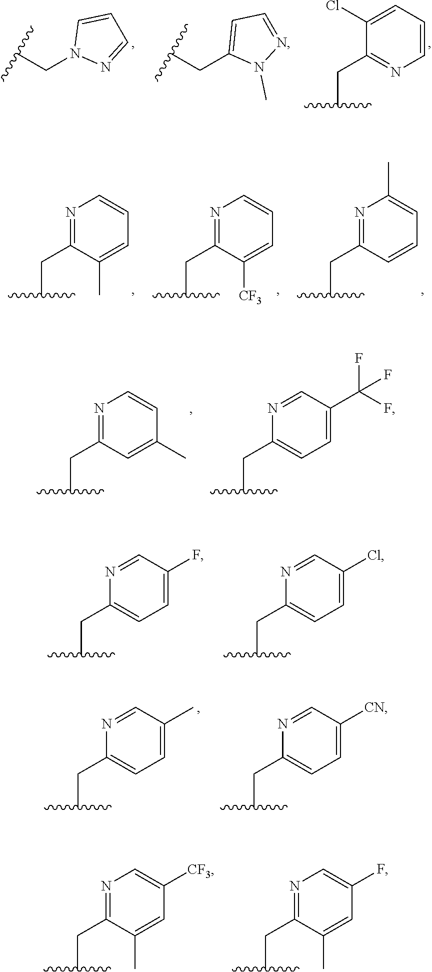

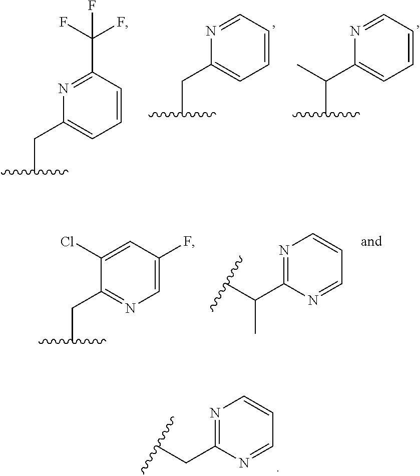

In some embodiments, R¹ is selected from the group consisting of

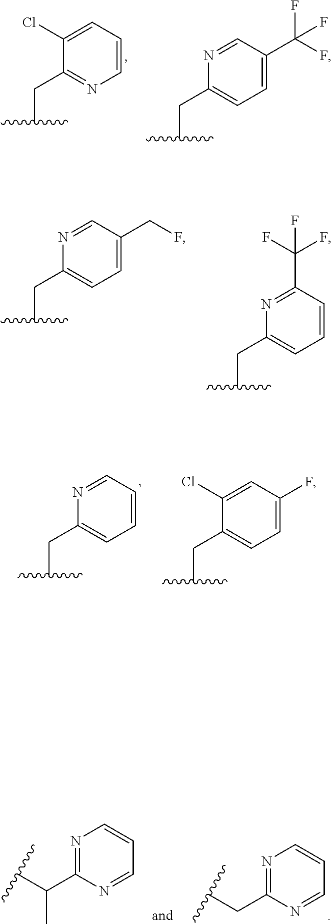

In some embodiments, R¹ is

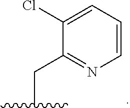 [SMILES: *Cc1ncccc1Cl]

In some embodiments, R¹ is

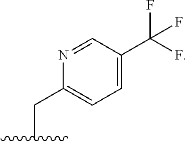 [SMILES: *Cc1ccc(C(F)(F)F)cn1]

In some embodiments, R¹ is

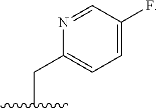 [SMILES: *Cc1ccc(F)cn1]

In some embodiments, R¹ is.

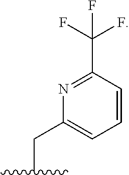 [SMILES: *Cc1cccc(C(F)(F)F)n1]

In some embodiments, R¹ is

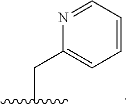 [SMILES: *Cc1ccccn1]

In some embodiments, R¹ is

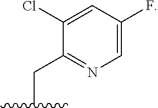 [SMILES: *Cc1ncc(F)cc1Cl]

In some embodiments, R¹ is

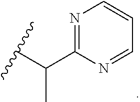 [SMILES: *C(C)c1ncccn1]

In some embodiments, R¹ is

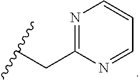 [SMILES: *Cc1ncccn1]

In some embodiments, R¹ is

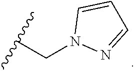 [SMILES: *Cn1cccn1]

In some embodiments, R¹ is

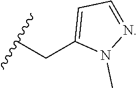 [SMILES: *Cc1ccnn1C]

In some embodiments, R¹ is

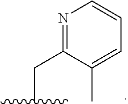 [SMILES: *Cc1ncccc1C]

In some embodiments, R¹ is

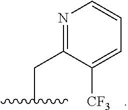 [SMILES: *Cc1ncccc1C(F)(F)F]

In some embodiments, R¹ is

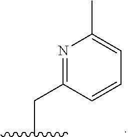 [SMILES: *Cc1cccc(C)n1]

In some embodiments, R¹ is

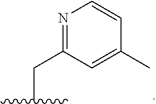 [SMILES: *Cc1cc(C)ccn1]

In some embodiments, R¹ is

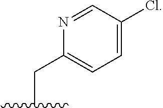 [SMILES: *Cc1ccc(Cl)cn1]

In some embodiments, R¹ is

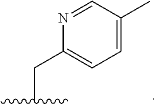 [SMILES: *Cc1ccc(C)cn1]

In some embodiments, R¹ is

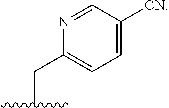 [SMILES: *Cc1ccc(C#N)cn1]

In some embodiments, R¹ is

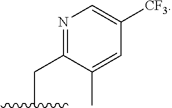 [SMILES: *Cc1ncc(C(F)(F)F)cc1C]

In some embodiments, R¹ is.

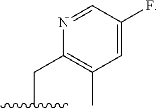 [SMILES: *Cc1ncc(F)cc1C]

In some embodiments, R¹ is

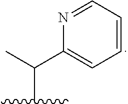 [SMILES: *C(C)c1ccccn1]

As generally defined herein, each R² is independently selected from the group consisting of -D, halo, ═O, —CN, —C₁-C₆ alkyl, —C₁-C₆ heteroalkyl, —C₁-C₆ haloalkyl, -C₃—C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂, wherein R^{a2} is as defined in any of the embodiments described herein.

In some embodiments, each R² is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}) and —OC(═O)N(R^{a2}), wherein R^{a2} is as defined in any of the embodiments described herein.

In some embodiments, each R² is independently selected from the group consisting of halo, ═O, —CN, —C₁-C₆ alkyl, —C₁-C₆ heteroalkyl, —C₁-C₆ haloalkyl, —C₃-C₉ cycloalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}) and —OC(═O)N(R^{a2})₂, wherein R^{a2} is as defined in any of the embodiments described herein. In some embodiments, each R² is independently selected from the group consisting of ═O, —C₁-C₆ alkyl, 3-10 membered heterocyclyl, —OR^{a2}, —C(═O)N(R^{a2})₂ and —N(R^{a2})₂, wherein R^{a2} is as defined in any of the embodiments described herein. In some embodiments, each R² is independently selected from the group consisting of ═O, —C₁-C₆ alkyl, —C₁-C₆ haloalkyl, —C₃-C₉ cycloalkyl, —OR^{a2} and —N(R^{a2})₂.

In some embodiments, each R^{a2} is independently selected from the group consisting of H and C₁-C₆ alkyl. In some embodiments, each R^{a2} is independently selected from the group consisting of H and -Me. In some embodiments, each R^{a2} is H.

In some embodiments, each R² is independently selected from the group consisting of ═O, —C₁-C₆ alkyl and —N(R^{a2})₂, wherein R^{a2} is as defined in any of the embodiments described herein.

In some embodiments, each R² is independently selected from the group consisting of -D, ═O, -Me, -Et, -ⁱPr, -^{t}Bu, —NH₂, —NHCH₃ and —NH(CH₃)₂.

In some embodiments, each R² is independently selected from the group consisting of ═O, -Me, -Et, —Pr, -^{t}Bu, —NH₂, —NHCH₃ and —NH(CH₃)₂.

In some embodiments, R² is independently selected from the group consisting of -D, —NH₂ and -Me.

In some embodiments, R² is independently selected from the group consisting of —NH₂ and -Me.

In some embodiments, R² is -D.

In some embodiments, R² is ═O.

In certain embodiments, R² is halo (e.g., fluoro, chloro, bromo, iodo). In some embodiments, R² is —Cl. In some embodiments, R² is —F. In some embodiments, R² is —Br. In some embodiments, R² is —I.

In some embodiments, R² is —CN.

In certain embodiments, R² is —C₁-C₆ alkyl. In some embodiments, R² is -Me. In some embodiments, R² is -Et. In some embodiments R² is —Pr or -iPr.

In some embodiments, R² is —C₁-C₆ heteroalkyl. In some embodiments, R² is methoxymethyl (—CH₂OCH₃). In some embodiments, R² is hydroxymethyl (—CH₂OH). In some embodiments, R² is aminomethyl (e.g., —CH₂NH₂, —CH₂NHCH₃, —CH₂N(CH₃)₂.

In some embodiments, R² is —C₁-C₆ haloalkyl. In some embodiments, R² is trifluoromethyl (—CF₃). In other embodiments, R² is difluoromethyl (—CHF₂).

In some embodiments, R² is C₃-C₉ cycloalkyl (e.g., cyclopropyl, cyclobutyl, cyclopentyl, cyclohexyl). In some embodiments, R² is cyclopropyl. In some embodiments R² is cyclobutyl. In some embodiments, R² is cyclopentyl. In some embodiments, R² is cyclohexyl.

In some embodiments, R² is 3-10 membered heterocyclyl. In some embodiments, R² is 3-6 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R² is oxetanyl (e.g., oxetan-3-yl). In some embodiments, R² is tetrahydropyranyl. In some embodiments, R² is tetrahydrofuranyl. In some embodiments, R² is azetidinyl. In some embodiments, R² is pyrrolidinyl. In some embodiments, R² is piperidinyl. In some embodiments, R² is piperazinyl. In some embodiments, R² is morpholinyl. In some embodiments, R² is azepanyl.

In some embodiments R² is cycloalkylalkyl (e.g., cyclopropylmethyl, cyclobutylmethyl, cyclopentylmethyl, cyclohexylmethyl, cycloheptylmethyl).

In some embodiments, R² is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, R² is arylalkyl. In some embodiments, R² is benzyl.

In some embodiments, R² is heteroarylalkyl (e.g., pyridinylmethyl, thiazolylmethyl, triazolylmethyl, pyrazolylmethyl).

In some embodiments, R² is —OR^{a2} wherein R^{a2} is as defined in any of the embodiments described herein (e.g., hydroxy (—OH), methoxy, ethoxy, propoxy, isopropoxy, cyclopropyloxy, cyclobutyloxy). In some embodiments, R² is hydroxy. In some embodiments, R² is methoxy. In some embodiments, R² is ethoxy. In some embodiments, R² is propoxy. In some embodiments, R² is isopropoxy. In some embodiments, R² is —C₁-C₆ haloalkoxy. In some embodiments, R² is trifluoromethoxy (—OCF₃), In other embodiments, R² is difluoromethoxy (—OCHF₂).

In some embodiments, R² is —N(R^{a2})₂ wherein R^{a2} is as defined in any of the embodiments described herein (e.g., —NH₂, —NHR^{a2}, —N(CH₃)Ra2). In some embodiments, R² is —NH₂. In some embodiments, R² is —NHR^{a2} (e.g., —NHCH₃, —NHCH₂CH₃, —NHPr, —NHⁱPr, -NHcyclopropyl, -NHcyclobutyl). In some embodiments, R² is —N(CH₃)R^{a2} (e.g., —N(CH₃)₂, —N(CH₃)CH₂CH₃, —N(CH₃)CH₂CH₂CH₃, —N(CH₃)′Pr, —N(CH₃)cyclopropyl, —N(CH₃)cyclobutyl).

In some embodiments, R² is —C(═O)R^{a2} or —C(═O)OR^{a2} wherein R^{a2} is as defined in any of the embodiments described herein. In some embodiments, R² is —C(═O)R^{a2} wherein R^{a2} is as defined in any of the embodiments described herein. In some embodiments, R² is —C(═O)alkyl. In some embodiments, R² is —C(═O)CH₃, —C(═O)cyclopropyl, —C(═O)cyclobutyl, —C(═O)^{t}Bu, —C(═O)′Pr, —C(═O)CH₂CH₂CH₃ or —C(═O)OCH₃. In some embodiments, R² is acetyl (—C(═O)CH₃). In some embodiments, R² is —C(═O)OR^{a2}. In some embodiments, R² is —COOH. In some embodiments, R² is COOCH₃.

In some embodiments, R² is —NR^{a2}C(═O)R^{a2} wherein R^{a2} is as defined in any of the embodiments described herein. In certain embodiments, R² is —NHC(═O)R^{a2} (e.g., —NHC(═O)CH₃, —NHC(═O)CH₂CH₃, —NHC(═O)CH₂CH₂CH₃, —NHC(═O)′Pr, —NHC(═O)Bu, —NHC(═O)^{t}Bu, —NHC(═O)Cyclopropyl, —NHC(═O)Cyclobutyl). In some embodiments, R² is —N(CH₃) C(═O)R^{a2} (e.g., —N(CH₃) C(═O)CH₃, —N(CH₃) C(═O)CH₂CH₃, —N(CH₃) C(═O)CH₂CH₂CH₃, —N(CH₃) C(═O) iPr, —N(CH₃) C(═O)Bu, —N(CH₃) C(═O)^{t}Bu, —N(CH₃) C(═O)Cyclopropyl, —N(CH₃) C(═O)Cyclobutyl).

In some embodiments, R² is —NR^{a2}C(═O)OR^{a2} wherein R^{a2} is as defined in any of the embodiments described herein. In certain embodiments, R² is —NHC(═O)OR^{a2} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O'Bu, —NHC(═O)OCyclopropyl, —NHC(═O)OCyclobutyl). In some embodiments, R² is —N(CH₃) C(═O)OR^{a2} (e.g., —N(CH₃) C(═O)OCH₃, —N(CH₃) C(═O)OCH₂CH₃, —N(CH₃) C(═O)OCH₂CH₂CH₃, —N(CH₃) C(═O)OⁱPr, —N(CH₃) C(═O)OBu, —N(CH₃) C(═O)O'Bu, —N(CH₃) C(═O)OCyclopropyl, —N(CH₃) C(═O)OCyclobutyl).

In some embodiments, R² is —C(═O)N(R^{a2})₂ wherein R^{a2} is as defined in any of the embodiments described herein (e.g., —C(═O)NH₂, —C(═O)NHR^{a2}, —C(═O)N(CH₃)Ra2). In some embodiments, R² is —C(═O)NH₂. In certain embodiments, R² is —C(═O)NHR^{a2} (e.g., —C(═O)NHCH₃, —C(═O)NHCH₂CH₃, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R² is —C(═O)N(CH₃)R^{a2} (e.g., —C(═O)N(CH₃)₂, —C(═O)N(CH₃)CH₂CH₃, —C(═O)N(CH₃)CH₂CH₂CH₃, —C(═O)N(CH₃)′Pr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R² is —C(═O)N(OR^{a2})(R^{a2}). In certain embodiments, R² is —C(═O)NH(OR^{a2})(e.g., —C(═O)NHOH, —C(═O)NHOCH₃). In some embodiments, R² is —C(═O)NHOH.

In some embodiments, R² is —OC(═O)N(R^{a2})₂ wherein R^{a2} is as defined in any of the embodiments described herein. In certain embodiments, R² is —OC(═O)NHR^{a2} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC (═O)NHCyclobutyl). In certain embodiments, R² is —OC(═O)N(CH₃)R^{a2} (e.g., —OC(═O)N(CH₃)₂, —OC(═O)N(CH₃)CH₂CH₃, —OC(═O)N(CH₃)CH₂CH₂CH₃, —OC(═O)N(CH₃)/Pr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R² is —S(═O)R^{a2} wherein R^{a2} is as defined in any of the embodiments described herein. In certain embodiments, R² is —S(═O)alkyl (e.g., —S(═O)CH₃, —S(═O)CH₂CH₃, —S(═O)CH₂CH₂CH₃, —S(═O)′Pr). In certain embodiments, R² is —S(═O)cycloalkyl (e.g., —S(═O)cyclopropyl, —S(═O)cyclobutyl, —S(═O)cyclopentyl, —S(═O)cyclohexyl).

In some embodiments, R² is —S(═O)₂R^{a2} wherein R^{a2} is as defined in any of the embodiments described herein. In certain embodiments, R² is —S(═O)₂alkyl (e.g., —S(═O)₂CH₃, —S(═O)₂CH₂CH₃, —S(═O)₂Pr, —S(═O)₂ⁱ Pr). In certain embodiments, R² is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R² is S(═O)₂aryl (e.g., —S(═O)₂phenyl).

In some embodiments, R² is —SR^{a2} wherein R^{a2} is as defined in any of the embodiments described herein. In certain embodiments, R² is -Salkyl (e.g., —SCH₃, —SCH₂CH₃, —SPr, —SⁱPr). In certain embodiments, R² is -Scycloalkyl (e.g., -Scyclopropyl, -Scyclobutyl,-Scyclopentyl, -Scyclohexyl). In certain embodiments, R² is -Saryl (e.g., -Sphenyl).

In some embodiments, R² is —S(═O)(═NR^{a2})R^{a2} wherein R^{a2} is as defined in any of the embodiments described herein. In certain embodiments, R² is —S(═O)(═NH)R^{a2} (e.g., —S(═O)(═NH)CH₃, —S(═O)(═NH)CH₂CH₃, —S(═O)(═NH)CH₂CH₂CH₃, —S(═O)(═NH)Pr, —S(═O)(═NH)Bu, —S(═O)(═NH)^{t}Bu, —S(═O)(═NH)Cyclopropyl, —S(═O)(═NH)Cyclobutyl). In some embodiments, R² is —S(═O)(═NCH₃)R^{a2} (e.g., —S(═O)(═NCH₃)CH₃, —S(═O)(═NCH₃)CH₂CH₃, —S(═O)(═NCH₃)CH₂CH₂CH₃, —S(═O)(═NCH₃)Pr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R² is —NR^{a2}S(═O)₂R^{a2} wherein R^{a2} is as defined in any of the embodiments described herein. In certain embodiments, R² is —NHS(═O)₂alkyl (e.g., —NHS(═O)₂CH₃, —NHS(═O)₂CH₂CH₃, —NHS(═O)₂Pr, —NHS(═O)₂/Pr). In certain embodiments, R² is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R² is —N(CH₃)S(═O)₂alkyl (e.g., —N(CH₃)S(═O)₂CH₃, —N(CH₃)S(═O)₂CH₂CH₃, —N(CH₃)S(═O)₂Pr, —N(CH₃)S(═O)₂ⁱ Pr). In certain embodiments, R² is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R² is —S(═O)₂N(R^{a2})₂ wherein R^{a2} is as defined in any of the embodiments described herein. (e.g., —S(═O)₂NH₂, —S(═O)₂NHR^{a2}, —S(═O)₂N(CH₃)Ra2). In some embodiments, R² is —S(═O)₂NH₂. In some embodiments, R² is —S(═O)₂NHR^{a2} (e.g., —S(═O)₂NHCH₃, —S(═O)₂NHCH₂CH₃, —S(═O)₂NHPr, —S(═O)₂NHⁱPr, —S(═O)₂NHcyclopropyl, —S(═O)₂NHcyclobutyl). In some embodiments, R² is —S(═O)₂N(CH₃)R^{a2} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)′Pr, —S(═O)₂N(CH₃)cyclopropyl, —S(═O)₂N(CH₃)cyclobutyl).

As generally defined herein, each R³ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂, wherein R^{a3} is as defined in any of the embodiments described herein.

In some embodiments, R³ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂ and —OC(═O)N(R^{a3})₂ wherein R^{a3} is as defined in any of the embodiments described herein.

In certain embodiments, R³ is selected from the group consisting of H, halo, —CN, —C₁-C₆ alkyl, —C₁-C₆ haloalkyl, —OR^{a3} and —N(R^{a3})₂ wherein R^{a3} is as defined in any of the embodiments described herein.

In some embodiments, R³ is selected from the group consisting of H, —C₁-C₆ alkyl, —C₁-C₆ haloalkyl, —OR^{a3} and —N(R^{a3})₂ wherein R^{a3} is as defined in any of the embodiments described herein.

In some embodiments, R³ is selected from the group consisting of OR^{a3} and —N(R^{a3}) 2 wherein R^{a3} is as defined in any of the embodiments described herein.

In some embodiments, each R^{a3} is independently selected from the group consisting of H, —C₁-C₆ alkyl (e.g., -Me, -Et, —Pr, —Pr, -nBu, -^{t}Bu, -sec-Bu, -iso-Bu) and —C₁-C₆ haloalkyl (e.g., —CHF2, —CF₃).

In some embodiments, R³ is selected from the group consisting of H, —C₁-C₆ alkyl (e.g., -Me, -Et, —Pr, -iPr, -nBu, -tBu, -sec-Bu, -iso-Bu), —C₁-C₆ alkyl (e.g., —CF₃, —CHF2), —OH, —O—(C₁-C₆ alkyl)(e.g., —OCH₃, -OEt), —O—(C₁-C₆ haloalkyl)(e.g., —OCF₃, —OCHF₂), —NH₂, —NH—(C₁-C₆ alkyl)(e.g., —NHCH₃) and —N—(C₁-C₆ alkyl)₂ (e.g, —N(CH₃)₂).

In certain embodiments, R³ is selected from the group consisting of H, -Me, -Et, —CHF2, —OCH₃, -OEt, —OCHF2, —OCF₃, —OH and —NH₂. In some embodiments, R³ is selected from the group consisting of H, -Et, —OCH₃, -OEt, —OCHF2, —OCF₃ and —OH.

In certain embodiments, R³ is selected from the group consisting of H, -Me, —CHF2, —OCH₃ and —NH2.

In other embodiments, R³ is selected from the group consisting of H, -Me, —CHF₂ and —NH₂. In some embodiments, R³ is selected from the group consisting of -Me and —NH₂.

In some embodiments, R³ is selected from the group consisting of H, —NH₂ and —OCH₃.

In some embodiments, R³ is selected from the group consisting of —NH₂ and —OCH₃.

In some embodiments, R³ is H. In some embodiments R³ is -D.

In certain embodiments, R³ is halo (e.g., fluoro, chloro, bromo, iodo). In some embodiments, R³ is —Cl. In some embodiments, R³ is —F. In some embodiments, R³ is —Br. In some embodiments, R³ is —I.

In some embodiments, R³ is —CN.

In certain embodiments, R³ is —C₁-C₆ alkyl. In some embodiments, R³ is -Me. In some embodiments, R³ is -Et. In some embodiments R³ is —Pr or -iPr.

In some embodiments, R³ is —C₁-C₆ heteroalkyl. In some embodiments, R³ is methoxymethyl (—CH₂OCH₃). In some embodiments, R³ is hydroxymethyl (—CH₂OH). In some embodiments, R³ is aminomethyl (e.g., —CH₂NH₂, —CH₂NHCH₃, —CH₂N(CH₃)₂.

In some embodiments, R³ is —C₁-C₆ haloalkyl. In some embodiments, R³ is trifluoromethyl (—CF₃). In other embodiments, R³ is difluoromethyl (—CHF₂).

In some embodiments, R³ is —C₃-C₉ cycloalkyl (e.g., cyclopropyl, cyclobutyl, cyclopentyl, cyclohexyl). In some embodiments, R³ is cyclopropyl. In some embodiments R3 is cyclobutyl. In some embodiments, R³ is cyclopentyl. In some embodiments, R³ is cyclohexyl.

In some embodiments, R³ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R³ is oxetanyl. In some embodiments, R³ is tetrahydropyranyl. In some embodiments, R³ is tetrahydrofuranyl. In some embodiments, R³ is azetidinyl. In some embodiments, R³ is pyrrolidinyl. In some embodiments, R³ is piperidinyl. In some embodiments, R³ is piperazinyl. In some embodiments, R³ is morpholinyl. In some embodiments, R³ is azepanyl.

In some embodiments R³ is cycloalkylalkyl (e.g., cyclopropylmethyl, cyclobutylmethyl, cyclopentylmethyl, cyclohexylmethyl, cycloheptylmethyl). In some embodiments, R³ is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, R³ is arylalkyl. In some embodiments, R³ is benzyl. In some embodiments, R³ is heteroarylalkyl (e.g., pyridinylmethyl, thiazolylmethyl, triazolylmethyl, pyrazolylmethyl).

In some embodiments, R³ is —OR^{a3} wherein R^{a3} is as defined in any of the embodiments described herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy). In some embodiments, R³ is hydroxy. In some embodiments, R³ is methoxy. In some embodiments, R³ is ethoxy. In some embodiments, R³ is propoxy. In some embodiments, R³ is isopropoxy. In some embodiments R³ is difluoromethoxy. (—OCHF₂). In some embodiments, R³ is trifluoromethoxy (—OCF₃).

In some embodiments, R³ is —N(R^{a3})₂ wherein R^{a3} is as defined in any of the embodiments described herein (e.g., —NH₂, —NHR^{a3}, —N(CH₃)Ra3). In some embodiments, R³ is —NH₂. In some embodiments, R³ is —NHR^{a3} (e.g., —NHCH₃, -NHEt, —NHPr, —NHⁱPr, -NHcyclopropyl,-NHcyclobutyl). In some embodiments, R³ is —N(CH₃)R^{a3} (e.g., —N(CH₃)₂, —N(CH₃) Et, —N(CH₃)Pr, —N(CH₃)ⁱPr, —N(CH₃)cyclopropyl, —N(CH₃)cyclobutyl). In some embodiments, R³ is —C(═O)R^{a3} or —C(═O)OR^{a3}. In some embodiments, R³ is —C(═O)R^{a3} wherein R^{a3} is as defined in any of the embodiments described herein. In some embodiments, R³ is —C(═O)alkyl. In some embodiments, R³ is —C(═O)CH₃, —C(═O)cyclopropyl, —C(═O)cyclobutyl, —C(═O)^{t}Bu, —C(═O)ⁱPr, —C(═O)Pr or —C(═O)OCH₃. In some embodiments, R³ is acetyl (—C(═O) Me). In some embodiments, R³ is —C(═O)OR^{a3}. In some embodiments, R³ is —COOH. In some embodiments, R³ is COOCH₃.

In some embodiments, R³ is —NR^{a3}C(═O)R^{a3} wherein R^{a3} is as defined in any of the embodiments described herein. In certain embodiments, R³ is —NHC(═O)R^{a3} (e.g., —NHC(═O) Me, —NHC(═O) Et, —NHC(═O)Pr, —NHC(═O)ⁱPr, —NHC(═O)Bu, —NHC(═O)^{t}Bu, —NHC(═O)Cyclopropyl, —NHC(═O)Cyclobutyl). In some embodiments, R³ is —N(CH₃) C(═O)R^{a3} (e.g., —N(CH₃) C(═O) Me, —N(CH₃) C(═O) Et, —N(CH₃) C(═O)Pr, —N(CH₃) C(═O) iPr, —N(CH₃) C(═O)Bu, —N(CH₃) C(═O)*Bu, —N(CH₃) C(═O)Cyclopropyl, —N(CH₃) C(═O)Cyclobutyl).

In some embodiments, R³ is —NR^{a3}C(═O)OR^{a3} wherein R^{a3} is as defined in any of the embodiments described herein. In certain embodiments, R³ is —NHC(═O)OR^{a3} (e.g., —NHC(═O)OCH₃, —NHC(═O)OEt, —NHC(═O)OPr, —NHC(═O)OⁱPr, —NHC(═O)OBu, —NHC(═O)O'Bu, —NHC(═O)OCyclopropyl, —NHC(═O)OCyclobutyl). In some embodiments, R³ is —N(CH₃) C(═O)OR^{a3} (e.g., —N(CH₃) C(═O)OCH₃, —N(CH₃) C(═O)OEt, —N(CH₃) C(═O)OPr, —N(CH₃) C(═O)OⁱPr, —N(CH₃) C(═O)OBu, —N(CH₃) C(═O)O'Bu, —N(CH₃) C(═O)OCyclopropyl, —N(CH₃) C(═O)OCyclobutyl).

In some embodiments, R³ is —C(═O)N(R^{a3})₂ wherein R^{a3} is as defined in any of the embodiments described herein (e.g., —C(═O)NH₂, —C(═O)NHR^{a3}, —C(═O)N(CH₃)Ra3). In some embodiments, R³ is —C(═O)NH₂. In certain embodiments, R³ is —C(═O)NHR^{a3} (e.g., —C(═O)NHCH₃, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R³ is —C(═O)N(CH₃)R^{a3} (e.g., —C(═O)N(CH₃)₂, —C(═O)N(CH₃) Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R³ is —OC(═O)N(R^{a3})₂ wherein R^{a3} is as defined in any of the embodiments described herein. In certain embodiments, R³ is —OC(═O)NHR^{a3} (e.g., —OC(═O)NHCH₃, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R³ is —OC(═O)N(CH₃)R^{a3} (e.g., —OC(═O)N(CH₃)₂, —OC(═O)N(CH₃) Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R³ is —S(═O)R^{a3} wherein R^{a3} is as defined in any of the embodiments described herein. In certain embodiments, R³ is —S(═O)alkyl (e.g., —S(═O) Me, —S(═O) Et, —S(═O)Pr, —S(═O)ⁱPr). In certain embodiments, R³ is —S(═O)cycloalkyl (e.g., —S(═O)cyclopropyl, —S(═O)cyclobutyl, —S(═O)cyclopentyl, —S(═O)cyclohexyl).

In some embodiments, R³ is —S(═O)₂R^{a3} wherein R^{a3} is as defined in any of the embodiments described herein. In certain embodiments, R³ is —S(═O)₂alkyl (e.g., —S(═O)₂Me, —S(═O)₂Et, —S(═O)₂Pr, —S(═O)₂ⁱ Pr). In certain embodiments, R³ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R³ is S(═O)₂aryl (e.g., —S(═O)₂phenyl).

In some embodiments, R³ is —SR^{a3} wherein R^{a3} is as defined in any of the embodiments described herein. In certain embodiments, R³ is -Salkyl (e.g., —SMe, -SEt, —SPr, —SⁱPr). In certain embodiments, R³ is -Scycloalkyl (e.g., -Scyclopropyl, -Scyclobutyl, -Scyclopentyl,-Scyclohexyl). In certain embodiments, R³ is -Saryl (e.g., -Sphenyl).

In some embodiments, R³ is —S(═O)(═NR^{a3})R^{a3} wherein R^{a3} is as defined in any of the embodiments described herein. In certain embodiments, R³ is —S(═O)(═NH)R^{a3} (e.g., —S(═O)(═NH) Me, —S(═O)(═NH) Et, —S(═O)(═NH)Pr, —S(═O)(═NH) iPr, —S(═O)(═NH)Bu, —S(═O)(═NH)^{t}Bu, —S(═O)(═NH)Cyclopropyl, —S(═O)(═NH)Cyclobutyl). In some embodiments, R³ is —S(═O)(═NCH₃)R^{a3} (e.g., —S(═O)(═NCH₃) Me, —S(═O)(═NCH₃) Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R³ is —NR^{a3}S(═O)₂R^{a3} wherein R^{a3} is as defined in any of the embodiments described herein. In certain embodiments, R³ is —NHS(═O)₂alkyl (e.g., —NHS(═O)₂Me, —NHS(═O)₂Et, —NHS(═O)₂Pr, —NHS(═O)₂ⁱ Pr). In certain embodiments, R³ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R³ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂ⁱ Pr). In certain embodiments, R³ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R³ is —S(═O)₂N(R^{a3})₂ wherein R^{a3} is as defined in any of the embodiments described herein. (e.g., —S(═O)₂NH₂, —S(═O)₂NHR^{a3}, —S(═O)₂N(CH₃)Ra3). In some embodiments, R³ is —S(═O)₂NH₂. In some embodiments, R³ is —S(═O)₂NHR^{a3} (e.g., —S(═O)₂NHCH₃, —S(═O)₂NHEt, —S(═O)₂NHPr, —S(═O)₂NHⁱPr, —S(═O)₂NHcyclopropyl, —S(═O)₂NHcyclobutyl). In some embodiments, R³ is —S(═O)₂N(CH₃)R^{a3} (e.g., —S(═O)₂N(CH₃)₂, —S(═O)₂N(CH₃) Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R⁴ is independently selected from the group consisting of -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂, wherein R^{a4} is as defined in any of the embodiments described herein.

In some embodiments, R⁴ is selected from the group consisting of halo, —CN, -C1-C6 alkyl, —C₁-C₆ heteroalkyl, —C₁-C₆ haloalkyl, —C₃-C₉ cycloalkyl (e.g., cyclopropyl), 3-10 membered heterocyclyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, wherein R^{a4} is as defined in any of the embodiments described herein.

In certain embodiments, R⁴ is selected from the group consisting of halo, —CN, -C1-C6 alkyl, —C₁-C₆ haloalkyl, 3-10 membered heterocyclyl (e.g., oxetanyl), —C₃-C₉ cycloalkyl (e.g., cyclopropyl), —OR^{a4}, —N(R^{a4})₂, —C(═O)R^{a4} and —C(═O)N(R^{a4})₂ wherein R^{a4} is as defined in any of the embodiments described herein.

In some embodiments, R⁴ is selected from the group consisting of halo, —C₁-C₆ alkyl, —C₁-C₆ haloalkyl, 3-10 membered heterocyclyl (e.g., oxetanyl), —C₃-C₉ cycloalkyl (e.g., cyclopropyl), —OR^{a4}, —C(═O)R^{a4} and —C(═O)N(R^{a4})₂, wherein R^{a4} is as defined in any of the embodiments described herein.

In some embodiments, R⁴ is selected from the group consisting of —C₁-C₆ alkyl, 3-10 membered heterocyclyl (e.g., oxetanyl), —C₃-C₉ cycloalkyl (e.g., cyclopropyl) and —C(═O)N(R^{a4})₂, wherein each R^{a4} is as defined in any of the embodiments described herein. In some embodiments, each R^{a4} is independently selected from the group consisting of H and —C₁-C₆ alkyl (e.g., -Me, -Et, —Pr, -iPr, -nBu, -tBu, -sec-Bu, -iso-Bu).

In some embodiments, R⁴ is selected from the group consisting of —Cl, -Me, -Et, -ⁱPr, —CF₃, —CHF2, —OCHF2, —OCF₃, cyclopropyl, —OCH₃, oxetan-3-yl, tetrahydrofuran-3-yl, —C(═O)NHOH, —C(═O) H and —C(═O)NH₂.

In certain embodiments, R⁴ is selected from the group consisting of —C₁-C₆ alkyl (e.g., -Me, -Et, —Pr, -iPr, -nBu, -tBu, -sec-Bu, -iso-Bu), 3-10 membered heterocyclyl (e.g., oxetan-3-yl), —C₃-C₉ cycloalkyl (e.g., cyclopropyl) and —C(═O)NH₂.

In some embodiments, R⁴ is selected from the group consisting of —Cl, -Me, -Et, -ⁱPr, —CF₃, —CHF2, —OCHF2, —OCF₃, and cyclopropyl. In some embodiments, R⁴ is selected from the group consisting of cyclopropyl, -Me and -Et.

In some embodiments, R⁴ is selected from the group consisting of -Me, -Et, oxetan-3-yl, cyclopropyl and —C(═O)NH₂.

In some embodiments, R⁴ is selected from the group consisting of -Me, -Et, cyclopropyl and —C(═O)NH₂.

In some embodiments, R⁴ is selected from the group consisting of -Me, -Et, oxetan-3-yl and cyclopropyl.

In some embodiments, R⁴ is selected from the group consisting of -Me, -Et and cyclopropyl.

In some embodiments, R⁴ is D.

In certain embodiments, R⁴ is halo (e.g., fluoro, chloro, bromo, iodo). In some embodiments, R⁴ is —Cl. In some embodiments, R⁴ is —F. In some embodiments, R⁴ is —Br. In some embodiments, R⁴ is —I.

In some embodiments, R⁴ is —CN.

In certain embodiments, R⁴ is —C₁-C₆ alkyl. In some embodiments, R⁴ is -Me. In some embodiments, R⁴ is -Et. In some embodiments R4 is —Pr or -iPr.

In some embodiments, R⁴ is —C₁-C₆ heteroalkyl. In some embodiments, R⁴ is methoxymethyl (—CH₂OCH₃). In some embodiments, R⁴ is hydroxymethyl (—CH₂OH). In some embodiments, R4 is aminomethyl (e.g., —CH₂NH₂, —CH₂NHCH₃, —CH₂N(CH₃)₂.

In some embodiments, R⁴ is —C₁-C₆ haloalkyl. In some embodiments, R⁴ is trifluoromethyl (—CF₃). In other embodiments, R⁴ is difluoromethyl (—CHF2).

In some embodiments, R⁴ is —C₃-C₉ cycloalkyl (e.g., cyclopropyl, cyclobutyl, cyclopentyl, cyclohexyl). In some embodiments, R⁴ is cyclopropyl. In some embodiments R4 is cyclobutyl. In some embodiments, R⁴ is cyclopentyl. In some embodiments, R⁴ is cyclohexyl.

In some embodiments, R⁴ is 3-6 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R⁴ is oxetanyl (e.g., oxetan-3-yl). In some embodiments, R⁴ is tetrahydropyranyl. In some embodiments, R⁴ is tetrahydrofuranyl. In some embodiments, R⁴ is azetidinyl. In some embodiments, R⁴ is pyrrolidinyl. In some embodiments, R⁴ is piperidinyl. In some embodiments, R⁴ is piperazinyl. In some embodiments, R⁴ is morpholinyl. In some embodiments, R⁴ is azepanyl.

In some embodiments R4 is cycloalkylalkyl (e.g., cyclopropylmethyl, cyclobutylmethyl, cyclopentylmethyl, cyclohexylmethyl, cycloheptylmethyl). In some embodiments, R⁴ is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, R⁴ is arylalkyl. In some embodiments, R⁴ is benzyl.

In some embodiments, R⁴ is heteroarylalkyl (e.g., pyridinylmethyl, thiazolylmethyl, triazolylmethyl, pyrazolylmethyl).

In some embodiments, R⁴ is —OR^{a4} wherein R^{a4} is as defined in any of the embodiments described herein (e.g., hydroxy (—OH), methoxy, ethoxy, propoxy, isopropoxy, cyclopropyloxy, cyclobutyloxy). In some embodiments, R⁴ is hydroxy. In some embodiments, R⁴ is methoxy. In some embodiments, R⁴ is ethoxy. In some embodiments, R⁴ is propoxy. In some embodiments, R⁴ is isopropoxy. In some embodiments, R⁴ is —C₁-C₆ haloalkoxy. In some embodiments, R⁴ is trifluoromethoxy (—OCF₃), In other embodiments, R⁴ is difluoromethoxy (—OCHF₂).

In some embodiments, R⁴ is —N(R^{a4})₂ wherein R^{a4} is as defined in any of the embodiments described herein (e.g., —NH₂, —NHR^{a4}, —N(CH₃)R^{a4}). In some embodiments, R⁴ is —NH₂. In some embodiments, R⁴ is —NHR^{a4} (e.g., —NHCH₃, -NHEt, —NHPr, —NHⁱPr, -NHcyclopropyl,-NHcyclobutyl). In some embodiments, R⁴ is —N(CH₃)R^{a4} (e.g., —N(CH₃)₂, —N(CH₃) Et, —N(CH₃)Pr, —N(CH₃)ⁱPr, —N(CH₃)cyclopropyl, —N(CH₃)cyclobutyl). In some embodiments, R⁴ is —C(═O)R^{a4} or —C(═O)OR^{a4} wherein R^{a4} is as defined in any of the embodiments described herein. In some embodiments, R⁴ is —C(═O)R^{a4} wherein R^{a4} is as defined in any of the embodiments described herein. In some embodiments, R⁴ is —C(═O)alkyl. In some embodiments, R⁴ is —C(═O)CH₃, —C(═O)cyclopropyl, —C(═O)cyclobutyl, —C(═O)^{t}Bu, —C(═O)ⁱPr, —C(═O)Pr or —C(═O)OCH₃. In some embodiments, R⁴ is acetyl (—C(═O) Me). In some embodiments, R⁴ is —C(═O)OR^{a4}. In some embodiments, R⁴ is —COOH. In some embodiments, R⁴ is COOCH₃.

In some embodiments, R⁴ is —NR^{a4}C(═O)R^{a4} wherein R^{a4} is as defined in any of the embodiments described herein. In certain embodiments, R⁴ is —NHC(═O)R^{a4} (e.g., —NHC(═O) Me, —NHC(═O) Et, —NHC(═O)Pr, —NHC(═O)ⁱPr, —NHC(═O)Bu, —NHC(═O)^{t}Bu, —NHC(═O)Cyclopropyl, —NHC(═O)Cyclobutyl). In some embodiments, R⁴ is —N(CH₃) C(═O)R^{a4} (e.g., —N(CH₃) C(═O) Me, —N(CH₃) C(═O) Et, —N(CH₃) C(═O)Pr, —N(CH₃) C(═O)ⁱPr, —N(CH₃) C(═O)Bu, —N(CH₃) C(═O)^{t}Bu, —N(CH₃) C(═O)Cyclopropyl, —N(CH₃) C(═O)Cyclobutyl).

In some embodiments, R⁴ is —NR^{a4}C(═O)OR^{a4} wherein R^{a4} is as defined in any of the embodiments described herein. In certain embodiments, R⁴ is —NHC(═O)OR^{a4} (e.g., —NHC(═O)OCH₃, —NHC(═O)OEt, —NHC(═O)OPr, —NHC(═O)OⁱPr, —NHC(═O)OBu, —NHC(═O)O'Bu, —NHC(═O)OCyclopropyl, —NHC (═O)OCyclobutyl). In some embodiments, R⁴ is —N(CH₃) C(═O)OR^{a4} (e.g., —N(CH₃) C(═O)OCH₃, —N(CH₃) C(═O)OEt, —N(CH₃) C(═O)OPr, —N(CH₃) C(═O)OⁱPr, —N(CH₃) C(═O)OBu, —N(CH₃) C(═O)O'Bu, —N(CH₃) C(═O)OCyclopropyl, —N(CH₃) C(═O)OCyclobutyl).

In some embodiments, R⁴ is —C(═O)N(R^{a4})₂ wherein R^{a4} is as defined in any of the embodiments described herein (e.g., —C(═O)NH₂, —C(═O)NHR^{a4}, —C(═O)N(CH₃)R^{a4}). In some embodiments, R⁴ is —C(═O)NH₂. In certain embodiments, R⁴ is —C(═O)NHR^{a4} (e.g., —C(═O)NHCH₃, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁴ is —C(═O)N(CH₃)R^{a4} (e.g., —C(═O)N(CH₃)₂, —C(═O)N(CH₃) Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R⁴ is —C(═O)N(OR^{a4})(R^{a4}) wherein R^{a4} is as defined in any of the embodiments described herein. In certain embodiments, R⁴ is —C(═O)NH(OR^{a4})(e.g., —C(═O)NHOH, —C(═O)NHOCH₃). In some embodiments, R⁴ is —C(═O)NHOH.

In some embodiments, R⁴ is —OC(═O)N(R^{a4})₂ wherein R^{a4} is as defined in any of the embodiments described herein. In certain embodiments, R⁴ is —OC(═O)NHR^{a4} (e.g., —OC(═O)NHCH₃, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁴ is —OC(═O)N(CH₃)R^{a4} (e.g., —OC(═O)N(CH₃)₂, —OC(═O)N(CH₃) Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁴ is —S(═O)R^{a4} wherein R^{a4} is as defined in any of the embodiments described herein. In certain embodiments, R⁴ is —S(═O)alkyl (e.g., —S(═O) Me, —S(═O) Et, —S(═O)Pr, —S(═O)ⁱPr). In certain embodiments, R⁴ is —S(═O)cycloalkyl (e.g., —S(═O)cyclopropyl, —S(═O)cyclobutyl, —S(═O)cyclopentyl, —S(═O)cyclohexyl).

In some embodiments, R⁴ is —S(═O)₂R^{a4} wherein R^{a4} is as defined in any of the embodiments described herein. In certain embodiments, R⁴ is —S(═O)₂alkyl (e.g., —S(═O)₂Me, —S(═O)₂Et, —S(═O)₂Pr, —S(═O)₂ⁱ Pr). In certain embodiments, R⁴ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁴ is S(═O)₂aryl (e.g., —S(═O)₂phenyl).

In some embodiments, R⁴ is —SR^{a4} wherein R^{a4} is as defined in any of the embodiments described herein. In certain embodiments, R⁴ is -Salkyl (e.g., —SMe, -SEt, —SPr, —SⁱPr). In certain embodiments, R⁴ is -Scycloalkyl (e.g., -Scyclopropyl, -Scyclobutyl, -Scyclopentyl,-Scyclohexyl). In certain embodiments, R⁴ is -Saryl (e.g., -Sphenyl).

In some embodiments, R⁴ is —S(═O)(═NR^{a4})R^{a4} wherein R^{a4} is as defined in any of the embodiments described herein. In certain embodiments, R⁴ is —S(═O)(═NH)R^{a4} (e.g., —S(═O)(═NH) Me, —S(═O)(═NH) Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R⁴ is —S(═O)(═NCH₃)R^{a4} (e.g., —S(═O)(═NCH₃) Me, —S(═O)(═NCH₃) Et, —S(═O)(═NCH₃)Pr, —S(═O)(═NCH₃)Pr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁴ is —NR^{a4}S(═O)₂R^{a4} wherein R^{a4} is as defined in any of the embodiments described herein. In certain embodiments, R⁴ is —NHS(═O)₂alkyl (e.g., —NHS(═O)₂Me, —NHS(═O)₂Et, —NHS(═O)₂Pr, —NHS(═O)₂ⁱ Pr). In certain embodiments, R⁴ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁴ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂ⁱ Pr). In certain embodiments, R⁴ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁴ is —S(═O)₂N(R^{a4})₂ wherein R^{a4} is as defined in any of the embodiments described herein. (e.g., —S(═O)₂NH₂, —S(═O)₂NHR^{a4}, —S(═O)₂N(CH₃)R^{a4}). In some embodiments, R⁴ is —S(═O)₂NH₂. In some embodiments, R⁴ is —S(═O)₂NHR^{a4} (e.g., —S(═O)₂NHCH₃, —S(═O)₂NHEt, —S(═O)₂NHPr, —S(═O)₂NHⁱPr, —S(═O)₂NHcyclopropyl, —S(═O)₂NHcyclobutyl). In some embodiments, R⁴ is —S(═O)₂N(CH₃)R^{a4} (e.g., —S(═O)₂N(CH₃)₂, —S(═O)₂N(CH₃) Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

Some embodiments of feature certain combinations of R³ and R⁴. In one embodiment, R³ is selected from the group consisting of H, —OCH₃, -OEt, —OCF₃, —OCHF2, —CHF2, -Me, -Et, —OH and —NH₂ and R⁴ is selected from the group consisting of —Cl, -Me, -Et, —Pr, —CF₃, —CHF2, —OCHF2, cyclopropyl and —C(═O)NH₂. In one embodiment, R³ is selected from the group consisting of H, —CHF2, -Me and —NH₂ and R⁴ is selected from the group consisting of —Cl, -Me, -Et, —CF₃, —CHF2, —OCHF2, oxetan-3-yl and cyclopropyl. In a further embodiment, R³ is selected from the group consisting of —NH₂ and -Me and R⁴ is selected from the group consisting of -Me, -Et, oxetan-3-yl and cyclopropyl.

In one embodiment, R³ is —NH₂ and R⁴ is selected from the group consisting of -Me,-Et, oxetan-3-yl and cyclopropyl. In some embodiments, R³ is —NH₂ and R⁴ is selected from the group consisting of -Me, -Et and cyclopropyl.

In another embodiment, R³ is selected from the group consisting of H, —OCH₃, -OEt, —OCF₃, —OCHF2, -Et and —OH and R⁴ is —C(═O)NH₂. In some embodiments, R³ is selected from the group consisting of H and —OCH₃ and R⁴ is —C(═O)NH₂. In some embodiments, R³ is —OCH₃ and R⁴ is —C(═O)NH₂.

As generally defined herein, each R⁵ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, -NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂, wherein R^{a5} is as defined in any of the embodiments described herein.

In some embodiments, R⁵ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a5}, —N(R^{a5})₂, —C(═O)R^{a5}, —C(═O)OR^{a5}, —NR^{a5}C(═O)R^{a5}, —NR^{a5}C (═O)OR^{a5}, —C(═O)N(R^{a5})₂ and —OC(═O)N(R^{a5}), wherein R^{a5} is as defined in any of the embodiments described herein.

In certain embodiments, R⁵ is selected from the group consisting of H, halo, —CN, —C₁-C₆ alkyl, —C₁-C₆ haloalkyl and —N(R^{a5})₂ wherein R^{a5} is as defined in any of the embodiments described herein. In some embodiments, R^{a5} is selected from the group consisting of H and —C₁-C₆ alkyl (e.g., -Me, -Et, —Pr, -iPr, -nBu, -tBu, -sec-Bu, -iso-Bu). In some embodiments, R⁵ is selected from the group consisting of —C₁-C₆ alkyl (e.g., -Me, -Et, —Pr, -iPr, -nBu, -tBu, -sec-Bu, -iso-Bu), —OH, —O—(C₁-C₆ alkyl)(e.g., —OCH₃), —NH₂, —NH—(C₁-C₆ alkyl)(e.g., —NHCH₃) and —N—(C₁-C₆ alkyl)₂ (e.g, —N(CH₃)₂). In some embodiments, R⁵ is selected from the group consisting of H, -Me and —NH₂. In certain embodiments, R⁵ is selected from the group consisting of H and -Me.

In some embodiments, R⁵ is H. In some embodiments R⁵ is -D.

In certain embodiments, R⁵ is halo (e.g., fluoro, chloro, bromo, iodo). In some embodiments, R⁵ is —Cl. In some embodiments, R⁵ is —F. In some embodiments, R⁵ is —Br. In some embodiments, R⁵ is —I.

In some embodiments, R⁵ is —CN.

In certain embodiments, R⁵ is —C₁-C₆ alkyl. In some embodiments, R⁵ is -Me. In some embodiments, R⁵ is -Et. In some embodiments R⁵ is —Pr or -iPr.

In some embodiments, R⁵ is —C₁-C₆ heteroalkyl. In some embodiments, R⁵ is methoxymethyl (—CH₂OCH₃). In some embodiments, R⁵ is hydroxymethyl (—CH₂OH). In some embodiments, R⁵ is aminomethyl (e.g., —CH₂NH₂, —CH₂NHCH₃, —CH₂N(CH₃)₂.

In some embodiments, R⁵ is —C₁-C₆ haloalkyl. In some embodiments, R⁵ is trifluoromethyl (—CF₃). In other embodiments, R⁵ is difluoromethyl (—CHF2).

In some embodiments, R⁵ is —C₃-C₉ cycloalkyl (e.g., cyclopropyl, cyclobutyl, cyclopentyl, cyclohexyl). In some embodiments, R⁵ is cyclopropyl. In some embodiments R⁵ is cyclobutyl. In some embodiments, R⁵ is cyclopentyl. In some embodiments, R⁵ is cyclohexyl.

In some embodiments, R⁵ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R⁵ is oxetanyl. In some embodiments, R⁵ is tetrahydropyranyl. In some embodiments, R⁵ is tetrahydrofuranyl. In some embodiments, R⁵ is azetidinyl. In some embodiments, R⁵ is pyrrolidinyl. In some embodiments, R⁵ is piperidinyl. In some embodiments, R⁵ is piperazinyl. In some embodiments, R⁵ is morpholinyl. In some embodiments, R⁵ is azepanyl.

In some embodiments R⁵ is cycloalkylalkyl (e.g., cyclopropylmethyl, cyclobutylmethyl, cyclopentylmethyl, cyclohexylmethyl, cycloheptylmethyl). In some embodiments, R⁵ is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, R⁵ is arylalkyl. In some embodiments, R⁵ is benzyl.

In some embodiments, R⁵ is heteroarylalkyl (e.g., pyridinylmethyl, thiazolylmethyl, triazolylmethyl, pyrazolylmethyl).

In some embodiments, R⁵ is —OR^{a5} wherein R^{a5} is as defined in any of the embodiments described herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy). In some embodiments, R⁵ is hydroxy. In some embodiments, R⁵ is methoxy. In some embodiments, R⁵ is ethoxy. In some embodiments, R⁵ is propoxy. In some embodiments, R⁵ is isopropoxy. In some embodiments R⁵ is difluoromethoxy. (—OCHF₂). In some embodiments, R⁵ is trifluoromethoxy (—OCF₃).

In some embodiments, R⁵ is —N(R^{a5})₂ wherein R^{a5} is as defined in any of the embodiments described herein (e.g., —NH₂, —NHR^{a5}, —N(CH₃)R^{a5}). In some embodiments, R⁵ is —NH₂. In some embodiments, R⁵ is —NHR^{a5} (e.g., —NHCH₃, -NHEt, —NHPr, —NHⁱPr, -NHcyclopropyl,-NHcyclobutyl). In some embodiments, R⁵ is —N(CH₃)R^{a5} (e.g., —N(CH₃)₂, —N(CH₃) Et, —N(CH₃)Pr, —N(CH₃)ⁱPr, —N(CH₃)cyclopropyl, —N(CH₃)cyclobutyl).

In some embodiments, R⁵ is —C(═O)R^{a5} or —C(═O)OR^{a5} wherein R^{a5} is as defined in any of the embodiments described herein. In some embodiments, R⁵ is —C(═O)R^{a5} wherein R^{a5} is as defined in any of the embodiments described herein. In some embodiments, R⁵ is —C(═O)alkyl. In some embodiments, R⁵ is —C(═O)CH₃, —C(═O)cyclopropyl, —C(═O)cyclobutyl, —C(═O)^{t}Bu, —C(═O)ⁱPr, —C(═O)Pr or —C(═O)OCH₃. In some embodiments, R⁵ is acetyl (—C(═O) Me). In some embodiments, R⁵ is —C(═O)OR^{a5}. In some embodiments, R⁵ is —COOH. In some embodiments, R⁵ is COOCH₃.

In some embodiments, R⁵ is —NR^{a5}C(═O)R^{a5} wherein R^{a5} is as defined in any of the embodiments described herein. In certain embodiments, R⁵ is —NHC(═O)R^{a5} (e.g., —NHC(═O) Me, —NHC(═O) Et, —NHC(═O)Pr, —NHC(═O)ⁱPr, —NHC(═O)Bu, —NHC(═O)^{t}Bu, —NHC(═O)Cyclopropyl, —NHC (═O)Cyclobutyl). In some embodiments, R⁵ is —N(CH₃) C(═O)R^{a5} (e.g., —N(CH₃) C(═O) Me, —N(CH₃) C(═O) Et, —N(CH₃) C(═O)Pr, —N(CH₃) C(═O)+Pr, —N(CH₃) C(═O)Bu, —N(CH₃) C(═O)^{t}Bu, —N(CH₃) C(═O)Cyclopropyl, —N(CH₃) C(═O)Cyclobutyl).

In some embodiments, R⁵ is —NR^{a5}C(═O)OR^{a5} wherein R^{a5} is as defined in any of the embodiments described herein. In certain embodiments, R⁵ is —NHC(═O)OR^{a5} (e.g., —NHC(═O)OCH₃, —NHC(═O)OEt, —NHC(═O)OPr, —NHC(═O)OⁱPr, —NHC(═O)OBu, —NHC(═O)O'Bu, —NHC(═O)OCyclopropyl, —NHC(═O)OCyclobutyl). In some embodiments, R⁵ is —N(CH₃) C(═O)OR^{a5} (e.g., —N(CH₃) C(═O)OCH₃, —N(CH₃) C(═O)OEt, —N(CH₃) C(═O)OPr, —N(CH₃) C(═O)OⁱPr, —N(CH₃) C(═O)OBu, —N(CH₃) C(═O)O'Bu, —N(CH₃) C(═O)OCyclopropyl, —N(CH₃) C(═O)OCyclobutyl).

In some embodiments, R⁵ is —C(═O)N(R^{a5})₂ wherein R^{a5} is as defined in any of the embodiments described herein (e.g., —C(═O)NH₂, —C(═O)NHR^{a5}, —C(═O)N(CH₃)R^{a5}). In some embodiments, R⁵ is —C(═O)NH₂. In certain embodiments, R⁵ is —C(═O)NHR^{a5} (e.g., —C(═O)NHCH₃, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁵ is —C(═O)N(CH₃)R^{a5} (e.g., —C(═O)N(CH₃)₂, —C(═O)N(CH₃) Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R⁵ is —OC(═O)N(R^{a5})₂ wherein R^{a5} is as defined in any of the embodiments described herein. In certain embodiments, R⁵ is —OC (═O)NHR^{a5} (e.g., —OC(═O)NHCH₃, —OC(═O)NHEt, —OC(═O)NHPr, —OC(═O)NHⁱPr, —OC(═O)NHBu, —OC(═O)NH^{t}Bu, —OC(═O)NHCyclopropyl, —OC (═O)NHCyclobutyl). In certain embodiments, R⁵ is —OC(═O)N(CH₃)R^{a5} (e.g., —OC(═O)N(CH₃)₂, —OC(═O)N(CH₃) Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁵ is —S(═O)R^{a5} wherein R^{a5} is as defined in any of the embodiments described herein. In certain embodiments, R⁵ is —S(═O)alkyl (e.g., —S(═O) Me, —S(═O) Et, —S(═O)Pr, —S(═O)ⁱPr). In certain embodiments, R⁵ is —S(═O)cycloalkyl (e.g., —S(═O)cyclopropyl, —S(═O)cyclobutyl, —S(═O)cyclopentyl, —S(═O)cyclohexyl).

In some embodiments, R⁵ is —S(═O)₂R^{a5} wherein R^{a5} is as defined in any of the embodiments described herein. In certain embodiments, R⁵ is —S(═O)₂alkyl (e.g., —S(═O)₂Me, —S(═O)₂Et, —S(═O)₂Pr, —S(═O)₂ⁱ Pr). In certain embodiments, R⁵ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁵ is S(═O) 2aryl (e.g., —S(═O)₂phenyl).

In some embodiments, R⁵ is —SR^{a5} wherein R^{a5} is as defined in any of the embodiments described herein. In certain embodiments, R⁵ is -Salkyl (e.g., —SMe, -SEt, —SPr, —SⁱPr). In certain embodiments, R⁵ is -Scycloalkyl (e.g., -Scyclopropyl, -Scyclobutyl, -Scyclopentyl,-Scyclohexyl). In certain embodiments, R⁵ is -Saryl (e.g., -Sphenyl).

In some embodiments, R⁵ is —S(═O)(═NR^{a5})R^{a5} wherein R^{a5} is as defined in any of the embodiments described herein. In certain embodiments, R⁵ is —S(═O)(═NH)R^{a5} (e.g., —S(═O)(═NH) Me, —S(═O)(═NH) Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R⁵ is —S(═O)(═NCH₃)R^{a5} (e.g., —S(═O)(═NCH₃) Me, —S(═O)(═NCH₃) Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁵ is —NR^{a5}S(═O)₂R^{a5} wherein R^{a5} is as defined in any of the embodiments described herein. In certain embodiments, R⁵ is —NHS(═O)₂alkyl (e.g., —NHS(═O)₂Me, —NHS(═O)₂Et, —NHS(═O)₂Pr, —NHS(═O)₂ⁱ Pr). In certain embodiments, R⁵ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁵ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂ⁱ Pr). In certain embodiments, R⁵ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁵ is —S(═O)₂N(R^{a5})₂ wherein R^{a5} is as defined in any of the embodiments described herein. (e.g., —S(═O)₂NH₂, —S(═O)₂NHR^{a5}, —S(═O)₂N(CH₃)R^{a5}). In some embodiments, R⁵ is —S(═O)₂NH₂. In some embodiments, R⁵ is —S(═O)₂NHR^{a5} (e.g., —S(═O)₂NHCH₃, —S(═O)₂NHEt, —S(═O)₂NHPr, —S(═O)₂NHⁱPr, —S(═O)₂NHcyclopropyl, —S(═O)₂NHcyclobutyl). In some embodiments, R⁵ is —S(═O)₂N(CH₃)R^{a5} (e.g., —S(═O)₂N(CH₃)₂, —S(═O)₂N(CH₃) Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R⁶ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂, wherein each R^{a6} is as defined in any of the embodiments described herein.

In certain embodiments, R⁶ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂ and —OC(═O)N(R^{a6})₂, wherein each R^{a6} is as defined in any of the embodiments described herein.

In some embodiments, R⁶ is selected from the group consisting of H, halo, —CN, —C₁-C₆ alkyl, —C₁-C₆ haloalkyl and —N(R^{a6})₂, wherein each R^{a6} is as defined in any of the embodiments described herein. In some embodiments, each R^{a6} is independently selected from the group consisting of H and —C₁-C₆ alkyl (e.g., -Me, -Et, —Pr, -iPr, -nBu, -tBu, -sec-Bu,-iso-Bu). In some embodiments, R⁶ is selected from the group consisting of —C₁-C₆ alkyl (e.g., -Me, -Et, —Pr, -iPr, -nBu, -tBu, -sec-Bu, -iso-Bu), —OH, —O—(C₁-C₆ alkyl)(e.g., —OCH₃), —NH₂, —NH—(C₁-C₆ alkyl)(e.g., —NHCH₃) and —N—(C₁-C₆ alkyl)₂ (e.g, —N(CH₃)₂). In some embodiments, R⁶ is H. In some embodiments R6 is -D.

In certain embodiments, R⁶ is halo (e.g., fluoro, chloro, bromo, iodo). In some embodiments, R⁶ is —Cl. In some embodiments, R⁶ is —F. In some embodiments, R⁶ is —Br. In some embodiments, R⁶ is —I.

In some embodiments, R⁶ is —CN.

In certain embodiments, R⁶ is —C₁-C₆ alkyl. In some embodiments, R⁶ is -Me. In some embodiments, R⁶ is -Et. In some embodiments R⁶ is —Pr or -iPr.

In some embodiments, R⁶ is —C₁-C₆ heteroalkyl. In some embodiments, R⁶ is methoxymethyl (—CH₂OCH₃). In some embodiments, R⁶ is hydroxymethyl (—CH₂OH). In some embodiments, R⁶ is aminomethyl (e.g., —CH₂NH₂, —CH₂NHCH₃, —CH₂N(CH₃)₂.

In some embodiments, R⁶ is —C₁-C₆ haloalkyl. In some embodiments, R⁶ is trifluoromethyl (—CF₃). In other embodiments, R⁶ is difluoromethyl (—CHF2). In some embodiments, R⁶ is —C₃-C₉ cycloalkyl (e.g., cyclopropyl, cyclobutyl, cyclopentyl, cyclohexyl). In some embodiments, R⁶ is cyclopropyl. In some embodiments R⁶ is cyclobutyl. In some embodiments, R⁶ is cyclopentyl. In some embodiments, R⁶ is cyclohexyl. In some embodiments, R⁶ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R⁶ is oxetanyl. In some embodiments, R⁶ is tetrahydropyranyl. In some embodiments, R⁶ is tetrahydrofuranyl. In some embodiments, R⁶ is azetidinyl. In some embodiments, R⁶ is pyrrolidinyl. In some embodiments, R⁶ is piperidinyl. In some embodiments, R⁶ is piperazinyl. In some embodiments, R⁶ is morpholinyl. In some embodiments, R⁶ is azepanyl.

In some embodiments R⁶ is cycloalkylalkyl (e.g., cyclopropylmethyl, cyclobutylmethyl, cyclopentylmethyl, cyclohexylmethyl, cycloheptylmethyl). In some embodiments, R⁶ is heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl).

In some embodiments, R⁶ is arylalkyl. In some embodiments, R⁶ is benzyl.

In some embodiments, R⁶ is heteroarylalkyl (e.g., pyridinylmethyl, thiazolylmethyl, triazolylmethyl, pyrazolylmethyl).

In some embodiments, R⁶ is —OR^{a6} wherein R^{a6} is as defined in any of the embodiments described herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy). In some embodiments, R⁶ is hydroxy. In some embodiments, R⁶ is methoxy. In some embodiments, R⁶ is ethoxy. In some embodiments, R⁶ is propoxy. In some embodiments, R⁶ is isopropoxy. In some embodiments R⁶ is difluoromethoxy. (—OCHF₂). In some embodiments, R⁶ is trifluoromethoxy (—OCF₃).

In some embodiments, R⁶ is —N(R^{a6})₂ wherein R^{a6} is as defined in any of the embodiments described herein (e.g., —NH₂, —NHR^{a6}, —N(CH₃)R^{a6}). In some embodiments, R⁶ is —NH₂. In some embodiments, R⁶ is —NHR^{a6} (e.g., —NHCH₃, -NHEt, —NHPr, —NHⁱPr, -NHcyclopropyl,-NHcyclobutyl). In some embodiments, R⁶ is —N(CH₃)R^{a6} (e.g., —N(CH₃)₂, —N(CH₃) Et, —N(CH₃)Pr, —N(CH₃)ⁱPr, —N(CH₃)cyclopropyl, —N(CH₃)cyclobutyl).

In some embodiments, R⁶ is —C(═O)R^{a6} or —C(═O)OR^{a6} wherein R^{a6} is as defined in any of the embodiments described herein. In some embodiments, R⁶ is —C(═O)R^{a6} wherein R^{a6} is as defined in any of the embodiments described herein. In some embodiments, R⁶ is —C(═O)alkyl. In some embodiments, R⁶ is —C(═O)CH₃, —C(═O)cyclopropyl, —C(═O)cyclobutyl, —C(═O)^{t}Bu, —C(═O)ⁱPr, —C(═O)Pr or —C(═O)OCH₃. In some embodiments, R⁶ is acetyl (—C(═O) Me). In some embodiments, R⁶ is —C(═O)OR^{a6}. In some embodiments, R⁶ is —COOH. In some embodiments, R⁶ is COOCH₃.

In some embodiments, R⁶ is —NR^{a6}C(═O)R^{a6} wherein R^{a6} is as defined in any of the embodiments described herein. In certain embodiments, R⁶ is —NHC(═O)R^{a6} (e.g., —NHC(═O) Me, —NHC(═O) Et, —NHC(═O)Pr, —NHC(═O)ⁱPr, —NHC(═O)Bu, —NHC(═O)^{t}Bu, —NHC(═O)Cyclopropyl, —NHC(═O)Cyclobutyl). In some embodiments, R⁶ is —N(CH₃) C(═O)R^{a6} (e.g., —N(CH₃) C(═O) Me, —N(CH₃) C(═O) Et, —N(CH₃) C(═O)Pr, —N(CH₃) C(═O)ⁱPr, —N(CH₃) C(═O)Bu, —N(CH₃) C(═O)^{t}Bu, —N(CH₃) C(═O)Cyclopropyl, —N(CH₃) C(═O)Cyclobutyl).

In some embodiments, R⁶ is —NR^{a6}C(═O)OR^{a6} wherein R^{a6} is as defined in any of the embodiments described herein. In certain embodiments, R⁶ is —NHC (═O)OR^{a6} (e.g., —NHC(═O)OCH₃, —NHC(═O)OEt, —NHC(═O)OPr, —NHC(═O)OⁱPr, —NHC(═O)OBu, —NHC(═O)O*Bu, —NHC(═O)OCyclopropyl, —NHC(═O)OCyclobutyl). In some embodiments, R⁶ is —N(CH₃) C(═O)OR^{a6} (e.g., —N(CH₃) C(═O)OCH₃, —N(CH₃) C(═O)OEt, —N(CH₃) C(═O)OPr, —N(CH₃) C(═O)OⁱPr, —N(CH₃) C(═O)OBu, —N(CH₃) C(═O)O'Bu, —N(CH₃) C(═O)OCyclopropyl, —N(CH₃) C(═O)OCyclobutyl).

In some embodiments, R⁶ is —C(═O)N(R^{a6})₂ wherein R^{a6} is as defined in any of the embodiments described herein (e.g., —C(═O)NH₂, —C(═O)NHR^{a6}, —C(═O)N(CH₃)R^{a6}). In some embodiments, R⁶ is —C(═O)NH₂. In certain embodiments, R⁶ is —C(═O)NHR^{a6} (e.g., —C(═O)NHCH₃, —C(═O)NHEt, —C(═O)NHPr, —C(═O)NH/Pr, —C(═O)NHBu, —C(═O)NH^{t}Bu, —C(═O)NHCyclopropyl, —C(═O)NHCyclobutyl). In certain embodiments, R⁶ is —C(═O)N(CH₃)R^{a6} (e.g., —C(═O)N(CH₃)₂, —C(═O)N(CH₃) Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R⁶ is —OC(═O)N(R^{a6})₂ wherein R^{a6} is as defined in any of the embodiments described herein. In certain embodiments, R⁶ is —OC(═O)NHR^{a6} (e.g., —OC(═O)NHCH₃, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁶ is —OC(═O)N(CH₃)R^{a6} (e.g., —OC(═O)N(CH₃)₂, —OC(═O)N(CH₃) Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁶ is —S(═O)R^{a6} wherein R^{a6} is as defined in any of the embodiments described herein. In certain embodiments, R⁶ is —S(═O)alkyl (e.g., —S(═O) Me, —S(═O)Et, —S(═O)Pr, —S(═O)ⁱPr). In certain embodiments, R⁶ is —S(═O)cycloalkyl (e.g., —S(═O)cyclopropyl, —S(═O)cyclobutyl, —S(═O)cyclopentyl, —S(═O)cyclohexyl).

In some embodiments, R⁶ is —S(═O)₂R^{a6} wherein R^{a6} is as defined in any of the embodiments described herein. In certain embodiments, R⁶ is —S(═O)₂alkyl (e.g., —S(═O)₂Me, —S(═O)₂Et, —S(═O)₂Pr, —S(═O) 2′Pr). In certain embodiments, R⁶ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁶ is S(═O)₂aryl (e.g., —S(═O)₂phenyl).

In some embodiments, R⁶ is —SR^{a6} wherein R^{a6} is as defined in any of the embodiments described herein. In certain embodiments, R⁶ is -Salkyl (e.g., —SMe, -SEt, —SPr, —SⁱPr). In certain embodiments, R⁶ is -Scycloalkyl (e.g., -Scyclopropyl, -Scyclobutyl, -Scyclopentyl,-Scyclohexyl). In certain embodiments, R⁶ is -Saryl (e.g., -Sphenyl).

In some embodiments, R⁶ is —S(═O)(═NR^{a6})R^{a6} wherein R^{a6} is as defined in any of the embodiments described herein. In certain embodiments, R⁶ is —S(═O)(═NH)R^{a6} (e.g., —S(═O)(═NH) Me, —S(═O)(═NH) Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R⁶ is —S(═O)(═NCH₃)R^{a6} (e.g., —S(═O)(═NCH₃) Me, —S(═O)(═NCH₃) Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁶ is —NR^{a6}S(═O)₂R^{a6} wherein R^{a6} is as defined in any of the embodiments described herein. In certain embodiments, R⁶ is —NHS(═O)₂alkyl (e.g., —NHS(═O)₂Me, —NHS(═O)₂Et, —NHS(═O)₂Pr, —NHS(═O)₂ⁱ Pr). In certain embodiments, R⁶ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl).

In certain embodiments, R⁶ is —N(CH₃)S(═O)₂alkyl wherein R^{a6} is as defined in any of the embodiments described herein (e.g., —N(CH₃)S(═O)₂Me, —N(CH₃)S(═O)₂Et, —N(CH₃)S(═O)₂Pr, —N(CH₃)S(═O)₂ⁱ Pr). In certain embodiments, R⁶ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁶ is —S(═O)₂N(R^{a6})₂ wherein R^{a6} is as defined in any of the embodiments described herein. (e.g., —S(═O)₂NH₂, —S(═O)₂NHR^{a6}, —S(═O)₂N(CH₃)R^{a6}). In some embodiments, R⁶ is —S(═O)₂NH₂. In some embodiments, R⁶ is —S(═O)₂NHR^{a6} (e.g., —S(═O)₂NHCH₃, —S(═O)₂NHEt, —S(═O)₂NHPr, —S(═O)₂NHⁱPr, —S(═O)₂NHcyclopropyl, —S(═O)₂NHcyclobutyl). In some embodiments, R⁶ is —S(═O)₂N(CH₃)R^{a6} (e.g., —S(═O)₂N(CH₃)₂, —S(═O)₂N(CH₃) Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, Ring B is selected from the group consisting of C₆-C₁₀ aryl and 5-10 membered heteroaryl, each optionally substituted at any available position.

In some embodiments, each aryl and heteroaryl of Ring B is substituted at any available position with 0, 1, 2 or 3 instances of R⁷, wherein each R⁷ is as defined in any of the embodiments described herein. In some embodiments, Ring B is substituted with 0, 1 or 2 instances of R⁷. In some embodiments, Ring B is substituted with 0 or 1 instances of R⁷. In some embodiments, Ring B is substituted with 1 or 2 instances of R⁷. In some embodiments, Ring B is unsubstituted. In some embodiments, Ring B is substituted with 1 instance of R⁷. In some embodiments, Ring B is substituted with 2 instances of R⁷. In some embodiments, Ring B is substituted with 3 instances of R⁷.

In some embodiments, Ring B is independently selected from the group consisting of —C₆-C₁₀ mono or bicyclic aryl (e.g., phenyl, fully aromatic 9-10 membered bicyclic aryl, bicyclic aryl containing a phenyl ring fused with a C₅-C₆ carbocycle, bicyclic aryl containing a phenyl ring fused with a 5-6 membered heterocycle containing 1-3 heteroatoms independently selected from the group consisting of N, O and S or oxidized forms thereof), 5-6 membered monocyclic heteroaryl (e.g., containing 1-4 heteroatoms independently selected from the group consisting of N, O and S) and an 8-10 membered bicyclic heteroaryl (e.g., containing 1-4 heteroatoms independently selected from the group consisting of N, O and S) wherein each aryl and heteroaryl is optionally substituted (e.g., substituted with 0, 1, 2 or 3 instances of R⁷ wherein R⁷ is as defined in any of the embodiments described herein).

In some embodiments, Ring B is selected from the group consisting of C₆-C₁₀ mono or bicyclic aryl (e.g., phenyl, naphthyl, 1,2,3,4-tetrahydronaphthalenyl, 2,3-dihydro-1H-indenyl, 1,2,3,4 tetrahydroquinolinyl, 1,2 dihydroquinolinyl, 1,2-dihydroisoquinolinyl, 1,2,3,4 tetrahydroisoquinolinyl, chromanyl, indolinyl, isoindolinyl, 3,4-dihydro-2H-benzo[b][1,4]oxazinyl, 2,3-dihydrobenzofuranyl, benzo[d][1,3]dioxolyl, 2,3-dihydro-1H-benzo[d]imidazolyl), 5-6 membered monocyclic heteroaryl (e.g., thiophenyl, thiazolyl, pyrazolyl, imidazolyl, oxazolyl, pyridinyl, pyrimidinyl), 8-10 membered bicyclic heteroaryl (e.g., benzo[d]isothiazolyl, indolyl, benzofuranyl, 1H-indazolyl, 2-H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, isoquinolinyl, benzo[d]imidazolyl, benzo[d]thiazolyl, benzo[d]oxazoly], [1,2,4]triazolo[4,3-a]pyridinyl, imidazo[1,2-a]pyridinyl, imidazo[1,5-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl) wherein each aryl and heteroaryl is optionally substituted (e.g., substituted with 0, 1, 2 or 3 instances of R⁷ wherein R⁷ is as defined in any of the embodiments described herein).

In some embodiments, Ring B is selected from the group consisting of thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl, chromanyl and 1,2,3,4-tetrahydro-1,8-naphthyridinyl), each optionally substituted (e.g., substituted with 0, 1, 2 or 3 instances of R⁷ wherein R⁷ is as defined in any of the embodiments described herein).

In some embodiments, Ring B is selected from the group consisting of thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl), each optionally substituted (e.g., substituted with 0, 1, 2 or 3 instances of R⁷ wherein R⁷ is as defined in any of the embodiments described herein).

In some embodiments, Ring B is selected from the group consisting of pyridinyl, pyrimidinyl, isoquinolinyl, pyrazolyl, chromanyl and phenyl, each optionally substituted (e.g., substituted with 0, 1, 2 or 3 instances of R⁷ wherein R⁷ is as defined in any of the embodiments described herein).

In some embodiments, Ring B is selected from the group consisting of pyridinyl, pyrimidinyl and phenyl, each optionally substituted (e.g., substituted with 0, 1, 2 or 3 instances of R⁷ wherein R⁷ is as defined in any of the embodiments described herein).

In some embodiments, Ring B is selected from the group consisting of pyrazol-5-yl, pyrazol-1-yl, thiophen-2-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, isoquinolin-1-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl, chroman-5-yl and thiazolo[5,4-b]pyridin-6-yl), each optionally substituted (e.g., substituted with 0, 1, 2 or 3 instances of R⁷ wherein R⁷ is as defined in any of the embodiments described herein).

In some embodiments, Ring B is selected from the group consisting of pyrazol-5-yl, thiophen-2-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted (e.g., substituted with 0, 1, 2 or 3 instances of R⁷ wherein R⁷ is as defined in any of the embodiments described herein).

In some embodiments, Ring B is selected from the group consisting of pyridin-2-yl, pyridin-3-yl, pyridin-4-yl, pyrimidin-2-yl, pyrazol-5-yl, pyrazol-1-yl, isoquinolin-1-yl, chroman-5-yl and phenyl, each optionally substituted (e.g., substituted with 0, 1, 2 or 3 instances of R⁷ wherein R⁷ is as defined in any of the embodiments described herein).

In some embodiments, Ring B is independently selected from the group consisting of a-C₆-C₁₀ mono or bicyclic aryl (e.g., phenyl, fully aromatic 9-10 membered bicyclic aryl, bicyclic aryl containing a phenyl ring fused with a C₅-C₆ carbocycle, bicyclic aryl containing a phenyl ring fused with a 5-6 membered heterocycle containing 1-3 heteroatoms independently selected from the group consisting of N, O and S or oxidized forms thereof) and an 8-10 membered bicyclic heteroaryl (e.g., containing 1-4 heteroatoms independently selected from the group consisting of N, O and S) wherein each aryl and heteroaryl is optionally substituted (e.g., substituted with 0, 1, 2 or 3 instances of R⁷ wherein R⁷ is as defined in any of the embodiments described herein).

In some embodiments, Ring B is independently selected from the group consisting of a-C₆-C₁₀ mono or bicyclic aryl (e.g., phenyl, fully aromatic 9-10 membered bicyclic aryl, bicyclic aryl containing a phenyl ring fused with a C₅-C₆ carbocycle, bicyclic aryl containing a phenyl ring fused with a 5-6 membered heterocycle containing 1-3 heteroatoms independently selected from the group consisting of N, O and S or oxidized forms thereof) and a 5-6 membered monocyclic heteroaryl (e.g., containing 1-4 heteroatoms independently selected from the group consisting of N, O and S) wherein each aryl and heteroaryl is optionally substituted (e.g., substituted with 0, 1, 2 or 3 instances of R⁷ wherein R⁷ is as defined in any of the embodiments described herein).

In some embodiments, Ring B is independently selected from the group consisting of phenyl and a 5-6 membered monocyclic heteroaryl (e.g., containing 1-4 heteroatoms independently selected from the group consisting of N, O and S) wherein the phenyl and the heteroaryl is optionally substituted (e.g., substituted with 0, 1, 2 or 3 instances of R⁷ wherein R⁷ is as defined in any of the embodiments described herein).

In some embodiments, Ring B is selected from the group consisting of pyridinyl, pyrimidinyl and phenyl, each optionally substituted (e.g., substituted with 0, 1, 2 or 3 instances of R⁷ wherein R⁷ is as defined in any of the embodiments described herein).

In some embodiments, Ring B is selected from the group consisting of pyridinyl and phenyl, each optionally substituted (e.g., substituted with 0, 1, 2 or 3 instances of R⁷ wherein R⁷ is as defined in any of the embodiments described herein).

In some embodiments, Ring B is selected from the group consisting of pyridin-2-yl, pyridin-3-yl, pyridin-4-yl, pyrimidin-2-yl and phenyl, each optionally substituted (e.g., substituted with 0, 1, 2 or 3 instances of R⁷ wherein R⁷ is as defined in any of the embodiments described herein).

In some embodiments, Ring B is selected from the group consisting of pyridin-2-yl and phenyl, each optionally substituted (e.g., substituted with 0, 1, 2 or 3 instances of R⁷ wherein R⁷ is as defined in any of the embodiments described herein).

In some embodiments, Ring B is unsubstituted. In some embodiments, Ring B is substituted with 1 instance of R⁷. In some embodiments, Ring B is substituted with 2 instances of R⁷. In some embodiments, Ring B is substituted with 3 instances of R⁷.

In some embodiments, Ring B is selected from the group consisting of:

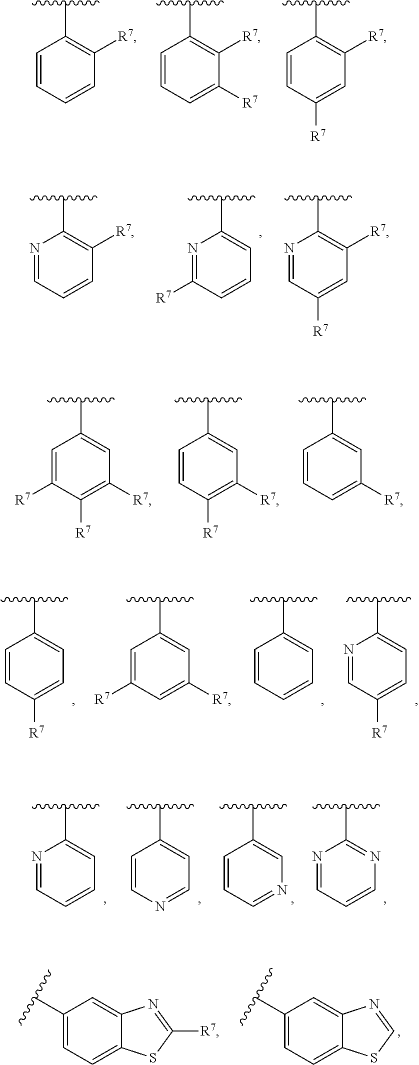

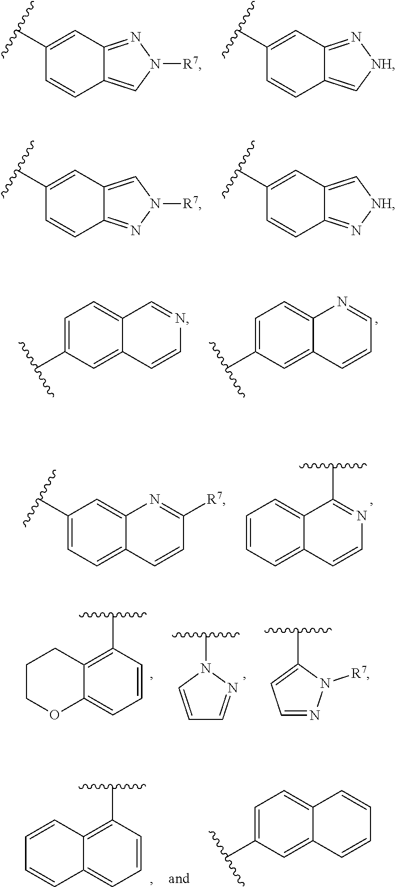

wherein each R⁷ is as defined in any of the embodiments described herein.

In some embodiments, Ring B is selected from the group consisting of:

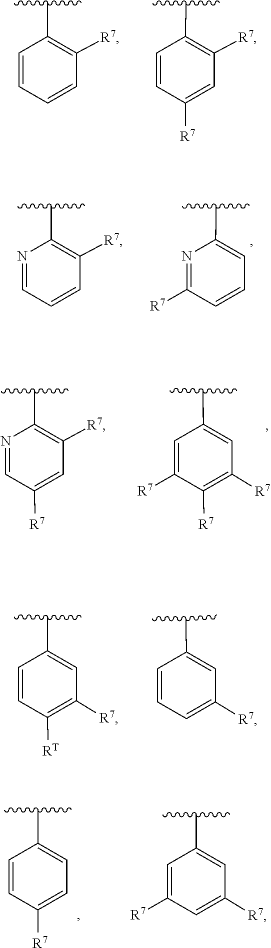

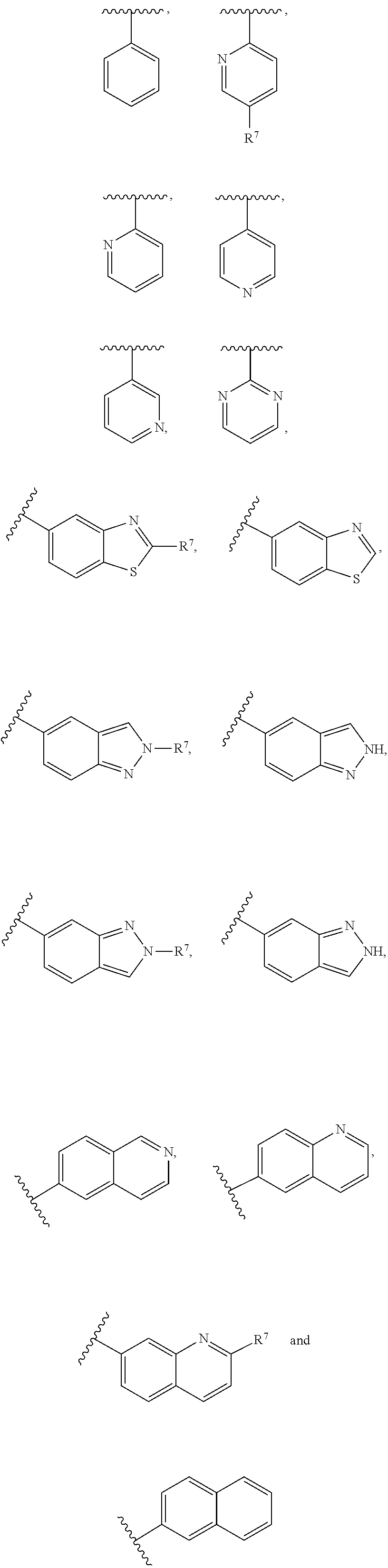

wherein each R⁷ is as defined in any of the embodiments described herein.

In some embodiments, Ring B is selected from the group consisting of:

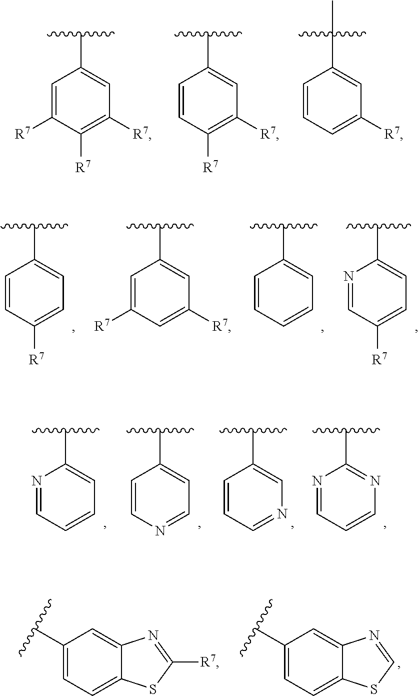

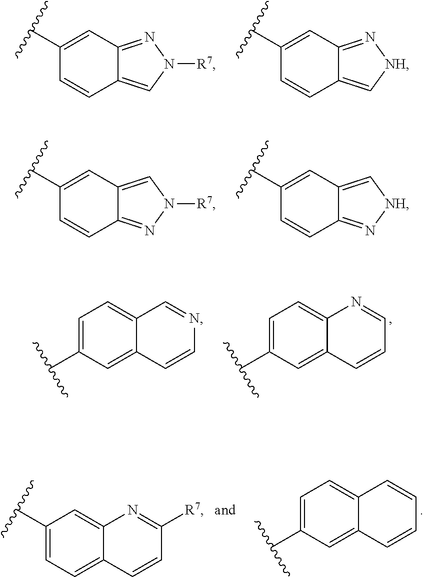

wherein each R⁷ is as defined in any of the embodiments described herein.

In some embodiments, Ring B is selected from the group consisting of:

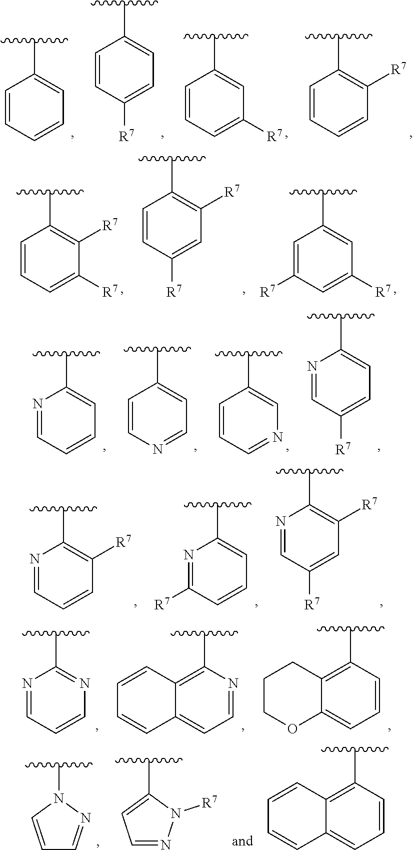

wherein each R⁷ is as defined in any of the embodiments described herein.

In some embodiments, Ring B is selected from the group consisting of

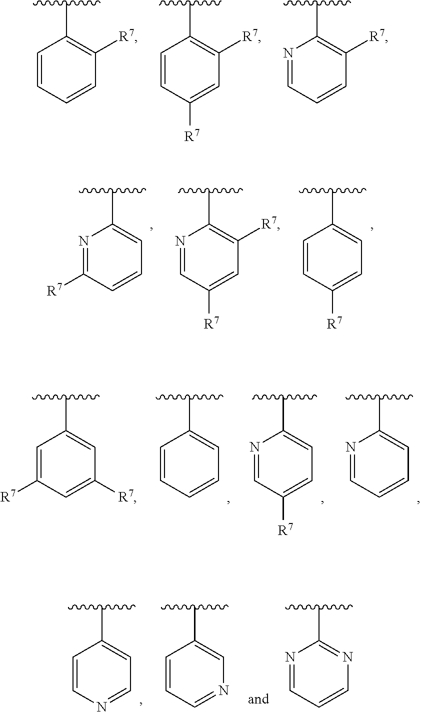

wherein each R⁷ is as defined in any of the embodiments described herein.

In some embodiments, Ring B is selected from the group consisting of

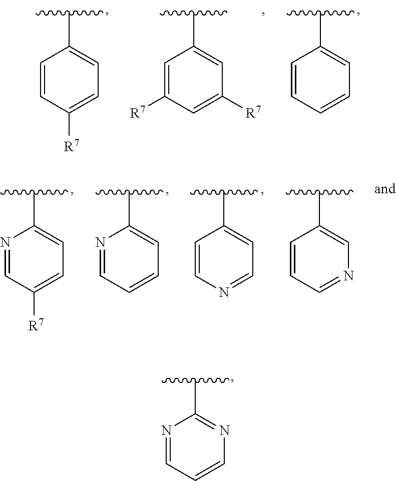

wherein each R⁷ is as defined in any of the embodiments described herein.

In some embodiments, Ring B is selected from the group consisting of:

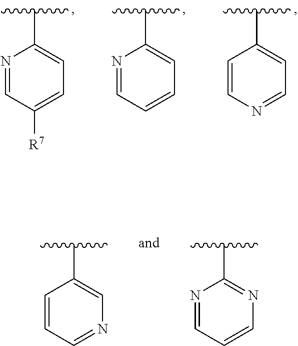

wherein each R⁷ is as defined in any of the embodiments described herein.

In some embodiments, Ring B is an optionally substituted 6-10 membered mono or bicyclic aryl. In some embodiments, Ring B is substituted with 0, 1, 2 or 3 instances of R⁷, wherein R⁷ is as defined in any of the embodiments described herein. In some embodiments, Ring B is naphthalenyl or phenyl, each optionally substituted at any available position (e.g., with 0, 1, 2 or 3 instances of R⁷, wherein R⁷ is as defined in any of the embodiments described herein).

In some embodiments, Ring B is optionally substituted phenyl. In some embodiments, Ring B is phenyl substituted with 0, 1, 2 or 3 instances of R⁷, wherein each R⁷ is independently as defined in any of the embodiments described herein. In some embodiments, the phenyl is unsubstituted. In some embodiments, the phenyl is substituted with one instance of R⁷. In some embodiments, the phenyl is substituted with 1 instance of R⁷ at the position ortho- to the attachment point. In some embodiments, the phenyl is substituted with 1 instance of R⁷ at the position para- to the attachment point. In some embodiments, the phenyl is substituted with 1 instance of R⁷ at the position meta- to the attachment point. In some embodiments, the phenyl is substituted with 2 instances of R⁷. In some embodiments, the phenyl is substituted with 3 instances of R⁷.

In some embodiments, Ring B is selected from the group consisting of:

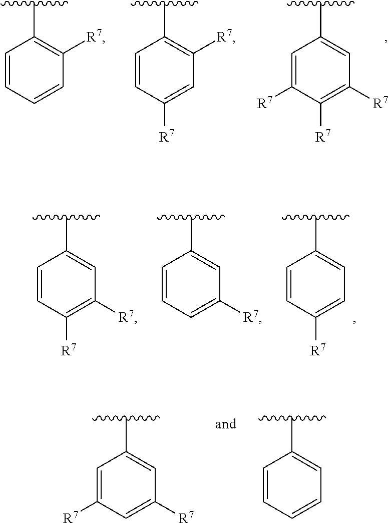

wherein each R⁷ is as defined in any of the embodiments described herein.

In some embodiments, Ring B is selected from the group consisting of:

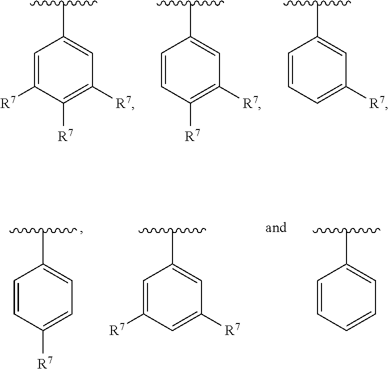

wherein each R⁷ is as defined in any of the embodiments described herein.

In some embodiments, Ring B is selected from the group consisting of:

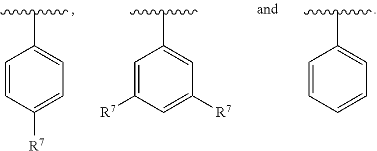

wherein each R⁷ is as defined in any of the embodiments described herein.

In some embodiments, Ring B is selected from the group consisting of:

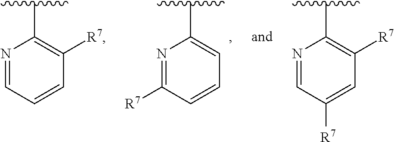

wherein each R⁷ is as defined in any of the embodiments described herein.

In some embodiments, Ring B is selected from the group consisting of:

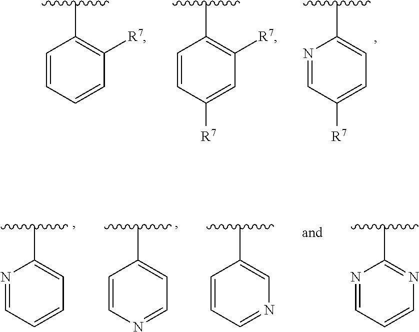

wherein each R⁷ is as defined in any of the embodiments described herein.

In some embodiments, Ring B is

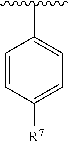

wherein each R⁷ is as defined in any of the embodiments described herein.

In some embodiments, Ring B is

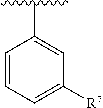

wherein each R⁷ is as defined in any of the embodiments described herein.

In some embodiments, Ring B is

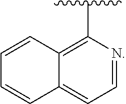 [SMILES: *c1nccc2ccccc12]

In some embodiments, Ring B is

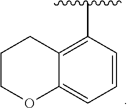 [SMILES: *c1cccc2c1CCCO2]

In some embodiments, Ring B is

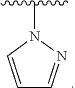 [SMILES: *n1cccn1 ; "."]

In some embodiments, Ring B is

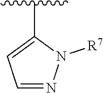

wherein each R⁷ is as defined in any of the embodiments described herein.

In some embodiments, Ring B is

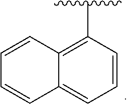 [SMILES: *c1cccc2ccccc12]

In some embodiments, Ring B is

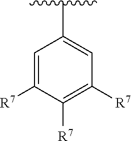

wherein each R⁷ is as defined in any of the embodiments described herein.

In some embodiments, Ring B is

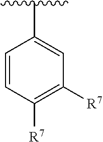

wherein each R⁷ is as defined in any of the embodiments described herein.

In some embodiments, Ring B is

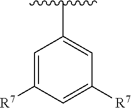

wherein each R⁷ is as defined in any of the embodiments described herein.

In some embodiments, Ring B is

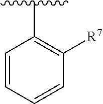

wherein each R⁷ is as defined in any of the embodiments described herein.

In some embodiments, Ring B

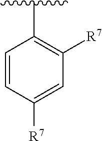

is wherein each R⁷ is as defined in any of the embodiments described herein.

In some embodiments, Ring B is

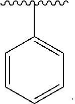 [SMILES: *c1ccccc1]

In some embodiments, Ring B is

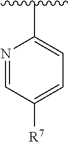

wherein each R⁷ is as defined in any of the embodiments described herein.

In some embodiments, Ring B is

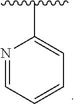 [SMILES: *c1ccccn1]

In some embodiments, Ring B is

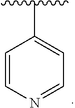 [SMILES: *c1ccncc1]

In some embodiments, Ring B is

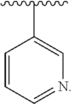 [SMILES: *c1cccnc1]

In some embodiments, Ring B is

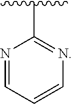 [SMILES: *c1ncccn1]

In some embodiments, Ring B is

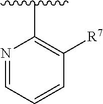

wherein each R⁷ is as defined in any of the embodiments described herein.

In some embodiments, Ring B

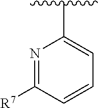

wherein each R⁷ is as defined in any of the embodiments described herein.

In some embodiments, Ring B is

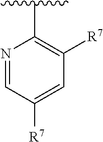

wherein each R⁷ is as defined in any of the embodiments described herein.

In some embodiments, Ring B is selected from the group consisting of:

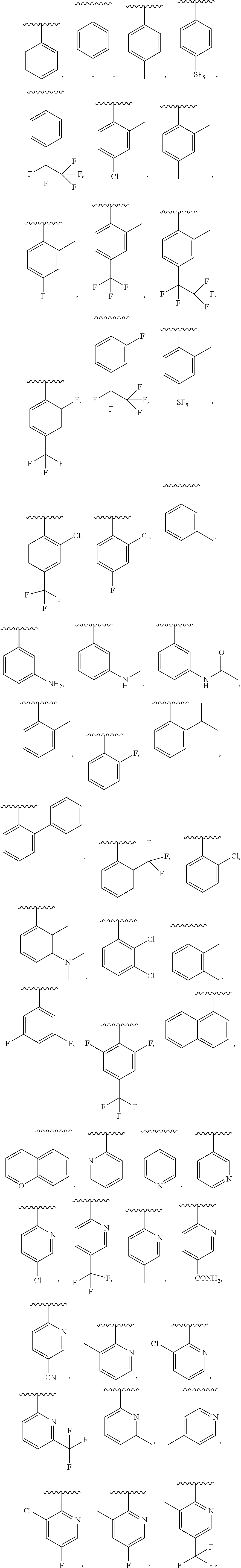

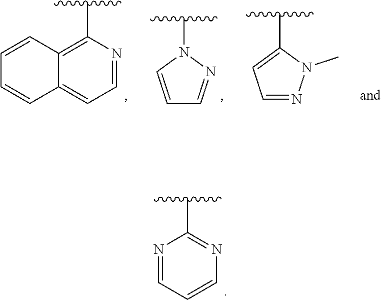

In some embodiments, Ring B is

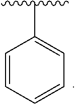 [SMILES: *c1ccccc1]

In some embodiments, Ring B is

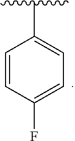 [SMILES: *c1ccc(F)cc1]

In some embodiments, Ring B is

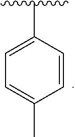 [SMILES: *c1ccc(C)cc1]

In some embodiments, Ring B is

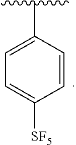

In some embodiments, Ring B is

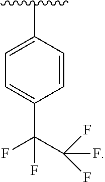 [SMILES: *c1ccc(C(F)(F)C(F)(F)F)cc1]

In some embodiments, Ring B is

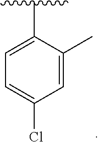 [SMILES: *c1ccc(Cl)cc1C]

In some embodiments, Ring B is

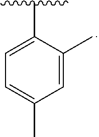 [SMILES: *c1ccc(C)cc1C]

In some embodiments, Ring B is

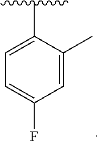 [SMILES: *c1ccc(F)cc1C]

In some embodiments, Ring B is

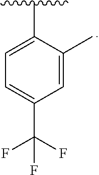 [SMILES: *c1ccc(C(F)(F)F)cc1C]

In some embodiments, Ring B is

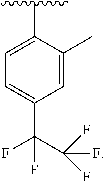 [SMILES: *c1ccc(C(F)(F)C(F)(F)F)cc1C]

In some embodiments, Ring B is

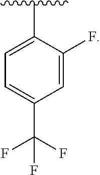 [SMILES: *c1ccc(C(F)(F)F)cc1F]

In some embodiments, Ring B is

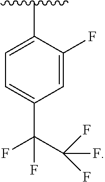 [SMILES: *c1ccc(C(F)(F)C(F)(F)F)cc1F]

In some embodiments, Ring B is

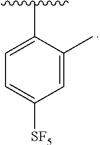

In some embodiments, Ring B is

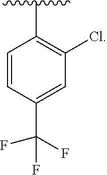 [SMILES: *c1ccc(C(F)(F)F)cc1Cl]

In some embodiments, Ring B is

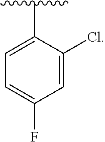 [SMILES: *c1ccc(F)cc1Cl]

In some embodiments, Ring B is

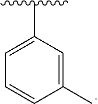 [SMILES: *c1cccc(C)c1]

In some embodiments, Ring B is

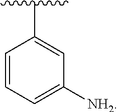 [SMILES: *c1cccc(N)c1]

In some embodiments, Ring B is

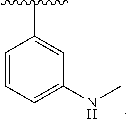 [SMILES: *c1cccc(NC)c1]

In some embodiments, Ring B is

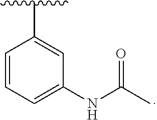 [SMILES: *c1cccc(NC(C)=O)c1]

In some embodiments, Ring B is

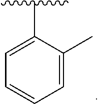 [SMILES: *c1ccccc1C]

In some embodiments, Ring B is

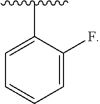 [SMILES: *c1ccccc1F]

In some embodiments, Ring B is

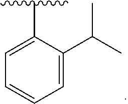 [SMILES: *c1ccccc1C(C)C]

In some embodiments, Ring B is

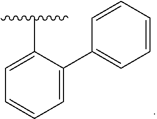 [SMILES: *c1ccccc1-c1ccccc1]

In some embodiments, Ring B is

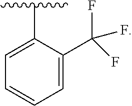 [SMILES: *c1ccccc1C(F)(F)F]

In some embodiments, Ring B

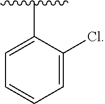 [SMILES: *c1ccccc1Cl]

In some embodiments, Ring B is

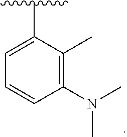 [SMILES: *c1cccc(N(C)C)c1C]

In some embodiments, Ring B is

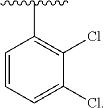 [SMILES: *c1cccc(Cl)c1Cl]

In some embodiments, Ring B is

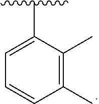 [SMILES: *c1cccc(C)c1C]

In some embodiments, Ring B is

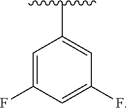 [SMILES: *c1cc(F)cc(F)c1]

In some embodiments, Ring B is

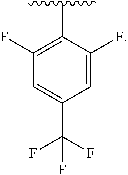 [SMILES: *c1c(F)cc(C(F)(F)F)cc1F]

In some embodiments, Ring B is

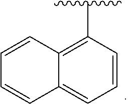 [SMILES: *c1cccc2ccccc12]

In some embodiments, Ring B is

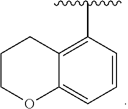 [SMILES: *c1cccc2c1CCCO2]

In some embodiments, Ring B is

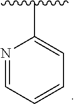 [SMILES: *c1ccccn1]

In some embodiments, Ring B is

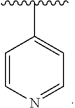 [SMILES: *c1ccncc1]

In some embodiments, Ring B is

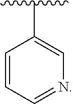 [SMILES: *c1cccnc1]

In some embodiments, Ring B is

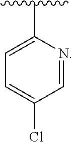 [SMILES: *c1ccc(Cl)cn1]

In some embodiments, Ring B is

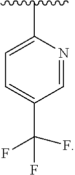 [SMILES: *c1ccc(C(F)(F)F)cn1]

In some embodiments, Ring B is

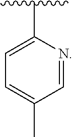 [SMILES: *c1ccc(C)cn1]

In some embodiments, Ring B is

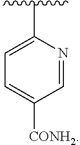 [SMILES: *c1ccc(C(N)=O)cn1]

In some embodiments, Ring B is

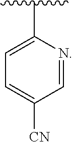 [SMILES: *c1ccc(C#N)cn1]

In some embodiments, Ring B is

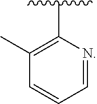 [SMILES: *c1ncccc1C]

In some embodiments, Ring B is

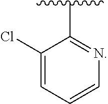 [SMILES: *c1ncccc1Cl]

In some embodiments, Ring B is

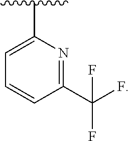 [SMILES: *c1cccc(C(F)(F)F)n1]

In some embodiments, Ring B is

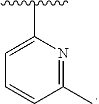 [SMILES: *c1cccc(C)n1]

In some embodiments, Ring B is

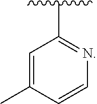 [SMILES: *c1cc(C)ccn1]

In some embodiments, Ring B is

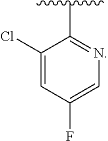 [SMILES: *c1ncc(F)cc1Cl]

In some embodiments, Ring B is

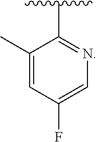 [SMILES: *c1ncc(F)cc1C]

In some embodiments, Ring B is

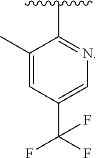 [SMILES: *c1ncc(C(F)(F)F)cc1C]

In some embodiments, Ring B is

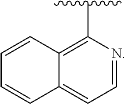 [SMILES: *c1nccc2ccccc12]

In some embodiments, Ring B is

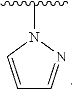 [SMILES: *n1cccn1]

In some embodiments, Ring B is

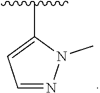 [SMILES: *c1ccnn1C]

In some embodiments, Ring B is

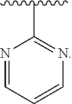 [SMILES: *c1ncccn1]

In some embodiments, the compounds of Formula (A) are of Formula (A-II):

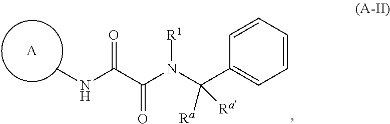

wherein R^{a}, R^{a}′, Ring A and R¹ are as defined in any of the embodiments described herein and the phenyl is substituted with 0, 1, 2 or 3 instances of R⁷ as defined in any of the embodiments described herein. In some embodiments, the compounds of Formula (I) are of Formula (II):

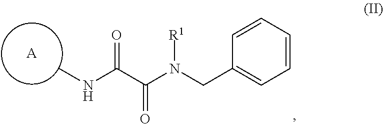

wherein Ring A and R¹ are as defined in any of the embodiments described herein and the phenyl is substituted with 0, 1, 2 or 3 instances of R⁷ as defined in any of the embodiments described herein. In some embodiments, the phenyl is unsubstituted. In some embodiments, the phenyl is substituted with one instance of R⁷. In some embodiments, the phenyl is substituted with 1 instance of R⁷ at the position para- to the attachment point to the piperidine. In some embodiments, the phenyl is substituted with 1 instance of R⁷ at the position meta- to the attachment point to the piperidine. In some embodiments, the phenyl is substituted with 2 instances of R⁷. In some embodiments, the phenyl is substituted with 3 instances of R⁷.

In some embodiments, compounds of Formula (A) are of Formula (A-II_1):

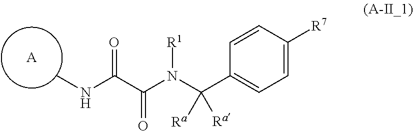

wherein R^{a}, R^{a}′, Ring A, R¹ and R⁷ are as defined in any of the embodiments described herein. In yet some embodiments, compounds of Formula (I) are of Formula (II_1):

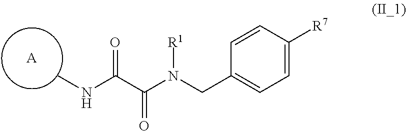

wherein Ring A, R¹ and R⁷ are as defined in any of the embodiments described herein.

In some embodiments, compounds of Formula (A) are of Formula (A-II_2):

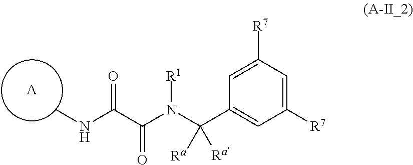

wherein R^{a}, R^{a}′, Ring A, R¹ and R⁷ are as defined in any of the embodiments described herein.

In other embodiments, compounds of Formula (I) are of Formula (II_2):

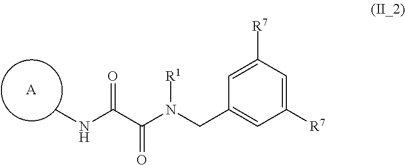

wherein Ring A, R¹ and R⁷ are as defined in any of the embodiments described herein.

In some embodiments, Ring B is phenyl substituted with halo (e.g., fluoro, chloro, bromo), —C₁-C₆ alkyl (e.g., -Me), —C₁-C₆ haloalkyl (e.g., —CF₃), —C₁-C₆ heteroalkoxy (e.g., —OCH₂CH₂N(CH₃)₂), or 3-10 member heterocyclyl (e.g., piperazinyl (e.g., N-Me piperazinyl)). In some embodiments, Ring B is phenyl substituted with —F, —Cl, -Me, —CF₃, —OCH₂CH₂N(CH₃)₂) or N-Me piperazinyl. In some embodiments, Ring B is phenyl substituted with halo (e.g., —F, —Cl, —Br). In some embodiments, Ring B is phenyl substituted with —Me. In some embodiments, Ring B is phenyl substituted with —CF₃.

In some embodiments, Ring B is an optionally substituted 9-10 membered bicyclic aryl (e.g., naphthalenyl). In some embodiments, Ring B is naphthalenyl (e.g., naphthalen-1-yl, naphthalen-2-yl). In some embodiments, Ring B is naphthalen-2-yl. In some embodiments, Ring B is an optionally substituted bicyclic aryl containing a phenyl ring fused with a C₅-C₆ carbocycle (e.g., tetrahydronaphthyl, dihydroindenyl). In some embodiments, Ring B is 1,2,3,4-tetrahydronaphthalenyl. In some embodiments, Ring B is 2,3-dihydro-1H-indenyl. In some embodiments, Ring B is an optionally substituted bicyclic aryl containing a phenyl ring fused with a 5-6 membered heterocycle containing 1-3 heteroatoms independently selected from the group consisting of N, O and S or oxidized forms thereof (e.g., tetrahydronaphthalenyl, dihydroindenyl, 1,2,3,4 tetrahydroquinolinyl, 1,2 dihydroquinolinyl, 1,2-dihydroisoquinolinyl, tetrahydroisoquinolinyl, chromanyl, indolinyl, isoindolinyl, dihydrobenzoxazinyl, dihydrobenzofuranyl, benzodioxolyl, dihydrobenzimidazolyl).

In some embodiments, the bicyclic aryl is unsubstituted. In some embodiments, Ring B is unsubstituted naphthalenyl (e.g., naphthalen-1-yl, naphthalen-2-yl). In some embodiments, Ring B is unsubstituted naphthalen-2-yl. In some embodiments, the bicyclic aryl is substituted with 0, 1, 2 or 3 instances of R⁷, wherein each R⁷ is as defined in any of the embodiments described herein. In some embodiments, the bicyclic aryl is substituted with 1 instance of R⁷. In some embodiments, the bicyclic aryl is substituted with one instance of R⁷ wherein R⁷ is selected from the group consisting of halo (e.g., —F, —CI, —Br), -Me, —O.

In some embodiments, Ring B is an optionally substituted 5-6 membered monocyclic heteroaryl (e.g., a 5-membered monocyclic heteroaryl containing 1-3 heteroatoms independently selected from the group consisting of O, N and S, a 6-membered monocyclic heteroaryl containing 1-3 N heteroatoms).

In some embodiments, the 5-6 membered monocyclic heteroaryl is unsubstituted. In some embodiments, the 5-6 membered monocyclic heteroaryl is substituted with 0, 1, 2 or 3 instances of R⁷, wherein each R⁷ is as defined in any of the embodiments described herein. In some embodiments, the 5-6 membered monocyclic heteroaryl is substituted with 1 instance of R⁷. In some embodiments, the 5-6 membered monocyclic heteroaryl is substituted with 2 instances of R⁷. In some embodiments, the 5-6 membered monocyclic heteroaryl is substituted with 2 instances of R⁷. In some embodiments, the 5-6 membered monocyclic heteroaryl is substituted with 3 instances of R⁷.

In some embodiments, Ring B is selected from the group consisting of thiophenyl, oxazolyl, isoxazolyl, thiazolyl, isothiazolyl, furanyl, pyrrolyl, pyrazolyl, imidazolyl, triazolyl, pyridinyl, pyrimidinyl, pyrazinyl and pyridazinyl, each optionally substituted (e.g., substituted at any available position with 0, 1, 2 or 3 instances of R⁷). In some embodiments, Ring B is selected from the group consisting of pyrazol-5-yl, pyrazol-1-yl, thiophene-2-yl, thiophen-3-yl, oxazol-5-yl, thiazol-5-yl, pyridin-2-yl, pyridin-3-yl, pyridin-4-yl and pyrimidin-2-yl, each optionally substituted (e.g., substituted at any available position with 0, 1, 2 or 3 instances of R⁷).

In some embodiments, Ring B is selected from the group consisting of pyrazol-5-yl, thiophene-2-yl, thiophen-3-yl, oxazol-5-yl, thiazol-5-yl, pyridin-2-yl, pyridin-3-yl, pyridin-4-yl and pyrimidin-2-yl, each optionally substituted (e.g., substituted at any available position with 0, 1, 2 or 3 instances of R⁷).

In some embodiments, Ring B is a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl) each optionally substituted (e.g., substituted at any available position with 0, 1, 2 or 3 instances of R⁷).

In some embodiments, Ring B is selected from the group consisting of thiophenyl, oxazolyl, isoxazolyl, thiazolyl, isothiazolyl, furanyl, pyrrolyl, pyrazolyl, imidazolyl and triazolyl, each optionally substituted (e.g., substituted at any available position with 0, 1, 2 or 3 instances of R⁷). In some embodiments, Ring B is selected from the group consisting of pyrazol-5-yl, pyrazol-1-yl, thiophene-2-yl, thiophen-3-yl, oxazol-5-yl and thiazol-5-yl each optionally substituted (e.g., substituted at any available position with 0, 1, 2 or 3 instances of R⁷).

In some embodiments, Ring B is pyrazolyl (e.g., pyrazol-3-yl, pyrazol-4-yl, pyrazol-5-yl). In some embodiments, Ring B is pyrrolyl (e.g., pyrrol-1-yl, pyrrol-2-yl, pyrrol-3-yl). In some embodiments, Ring B is thiophenyl (e.g., thiophen-2-yl, thiophen-3-yl). In some embodiments, Ring B is furyl (e.g., fur-2-yl, fur-3-yl). In some embodiments, Ring B is thiazolyl (e.g., thiazol-2-yl, thiazol-4-yl, thiazol-5-yl). In some embodiments, Ring B is isothiazolyl (e.g., isothiazol-3-yl, isothiazol-4-yl, isothiazol-5-yl). In some embodiments, Ring B is oxazolyl (e.g., oxazol-2-yl, oxazol-4-yl, oxazol-5-yl). In some embodiments, Ring B is isoxazolyl (e.g., isoxazol-3-yl, isoxazol-4-yl, isoxazol-5-yl). In some embodiments, Ring B is imidazolyl (e.g., imidazol-2-yl, imidazol-4-yl). In some embodiments, Ring B is triazolyl. In some embodiments, Ring B is thiadiazolyl. In some embodiments, Ring B is oxadiazolyl. In certain embodiments, the 5-membered monocyclic heteroaryl is unsubstituted. In some embodiments, the 5-membered monocyclic heteroaryl is substituted with 1 instance of R⁷. In some embodiments, the 5-membered monocyclic heteroaryl is substituted with 2 instances of R⁷. In some embodiments, the 5-membered monocyclic heteroaryl is substituted with 3 instances of R⁷.

In some embodiments, Ring B is a 6-membered monocyclic heteroaryl (e.g., pyridyl, pyrimidinyl, triazinyl, pyrazinyl, pyridazinyl). In some embodiments, the 6-membered monocyclic heteroaryl is unsubstituted. In some embodiments, the 6-membered monocyclic heteroaryl is substituted with 0, 1, 2 or 3 instances of R⁷. In some embodiments, the 6-membered monocyclic heteroaryl is substituted with 1 instance of R⁷. In some embodiments, the 6-membered monocyclic heteroaryl is substituted with 2 instances of R⁷. In some embodiments, the 6-membered monocyclic heteroaryl is substituted with 3 instances of R⁷. In some embodiments, Ring B is selected from the group consisting of pyridinyl (e.g., pyridin-2-yl, pyridin-3-yl, pyridin-4-yl) and pyrimidinyl(e.g, pyrimidin-2-yl, pyrimidin-4-yl, pyrimidin-5-yl) each optionally substituted (e.g, substituted at any available position with 0, 1, 2 or 3 instances of R⁷). In some embodiments, Ring B is selected from the group consisting of pyridin-2-yl, pyridin-3-yl, pyridin-4-yl and pyrimidin-2-yl, each optionally substituted (e.g, substituted at any available position with 0, 1, 2 or 3 instances of R⁷). In some embodiments, Ring B is selected from the group consisting of pyridin-2-yl and pyrimidin-2-yl, each optionally substituted (e.g, substituted at any available position with 0, 1, 2 or 3 instances of R⁷). In some embodiments, Ring B is pyridinyl (e.g., pyridin-2-yl, pyridin-3-yl, pyridin-4-yl), optionally substituted (e.g, substituted at any available position with 0, 1, 2 or 3 instances of R⁷. In some embodiments, Ring B is pyridin-2-yl, optionally substituted (e.g, substituted at any available position with 0, 1, 2 or 3 instances of R⁷. In some embodiments, Ring B is pyridin-3-yl, optionally substituted (e.g, substituted at any available position with 0, 1, 2 or 3 instances of R⁷. In some embodiments, Ring B is pyridin-4-yl, optionally substituted (e.g, substituted at any available position with 0, 1, 2 or 3 instances of R⁷. In some embodiments, Ring B is pyrimidinyl(e.g, pyrimidin-2-yl, pyrimidin-4-yl, pyrimidin-5-yl), optionally substituted (e.g, substituted at any available position with 0, 1, 2 or 3 instances of R⁷. In some embodiments, Ring B is pyrimidin-2-yl, optionally substituted (e.g, substituted at any available position with 0, 1, 2 or 3 instances of R⁷. In some embodiments, Ring B is pyrimidin-4-yl. In some embodiments, Ring B is pyrimidin-5-yl, optionally substituted (e.g, substituted at any available position with 0, 1, 2 or 3 instances of R⁷.

In one embodiment, Ring B is

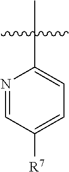

wherein R⁷ is as defined in any of the embodiments described herein. In one embodiment, Ring B is

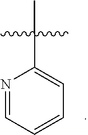 [SMILES: *c1ccccn1]

In one embodiment, Ring B is

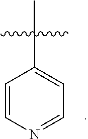 [SMILES: *c1ccncc1]

In one embodiment, Ring B is

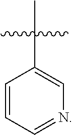 [SMILES: *c1cccnc1]

In one embodiment, Ring B is

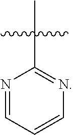 [SMILES: *c1ncccn1]

In some embodiments, the compounds of Formula (A) are of Formula (A-III):

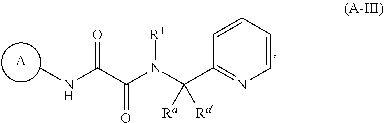

wherein R^{a}, R^{a}′, Ring A and R¹ are as defined in any of the embodiments described herein and the pyridinyl is substituted with 0, 1, 2 or 3 instances of R⁷ as defined in any of the embodiments described herein.

In some embodiments, the compounds of Formula (I) are of Formula (III):

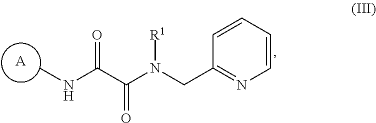

wherein Ring A and R¹ are as defined in any of the embodiments described herein and the pyridinyl is substituted with 0, 1, 2 or 3 instances of R⁷ as defined in any of the embodiments described herein. In some embodiments, the pyridinyl is unsubstituted. In some embodiments, the pyridinyl is substituted with one instance of R⁷. In some embodiments, the pyridinyl is substituted with 1 instance of R⁷ at the 5-position. In some embodiments, the pyridinyl is substituted with 2 instances of R⁷. In some embodiments, the pyridinyl is substituted with 3 instances of R⁷.

In some embodiments, the compounds of Formula (A) are of Formula (A-III_1):

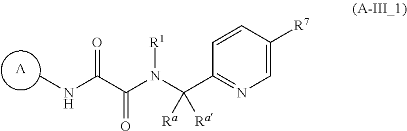

wherein R^{a}, R^{a}′, Ring A, R¹ and R⁷ are as defined in any of the embodiments described herein.

In some embodiments, the compounds of Formula (I) are of Formula (III_1):

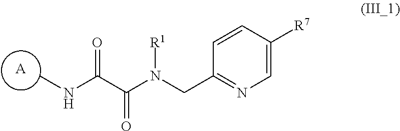

wherein Ring A, R¹ and R⁷ are as defined in any of the embodiments described herein.

In some embodiments, the compounds of Formula (A) are of Formula (A-IV):

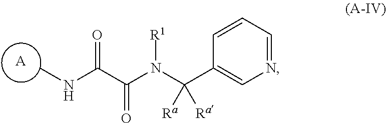

wherein R^{a}, R^{a}′, Ring A and R^{I} are as defined in any of the embodiments described herein and the pyridinyl is substituted with 0, 1, 2 or 3 instances of R⁷ as defined in any of the embodiments described herein.

In some embodiments, the compounds of Formula (I) are of Formula (IV):

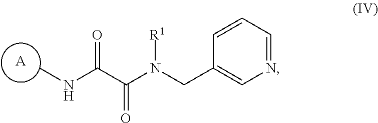

wherein Ring A and R^{I} are as defined in any of the embodiments described herein and the pyridinyl is substituted with 0, 1, 2 or 3 instances of R⁷ as defined in any of the embodiments described herein. In some embodiments, the pyridinyl is unsubstituted. In some embodiments, the pyridinyl is substituted with one instance of R⁷. In some embodiments, the pyridinyl is substituted with 2 instances of R⁷. In some embodiments, the pyridinyl is substituted with 3 instances of R⁷.

In some embodiments, the compounds of Formula (A) are of Formula (A-V):

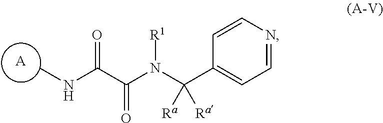

wherein R^{a}, R^{a}′, Ring A and R¹ are as defined in any of the embodiments described herein and the pyridinyl is substituted with 0, 1, 2 or 3 instances of R⁷ as defined in any of the embodiments described herein.

In some embodiments, the compounds of Formula (I) are of Formula (V):

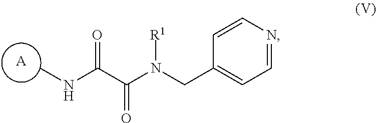

wherein Ring A and R¹ are as defined in any of the embodiments described herein and the pyridinyl is substituted with 0, 1, 2 or 3 instances of R⁷ as defined in any of the embodiments described herein. In some embodiments, the pyridinyl is unsubstituted. In some embodiments, the pyridinyl is substituted with one instance of R⁷. In some embodiments, the pyridinyl is substituted with 2 instances of R⁷. In some embodiments, the pyridinyl is substituted with 3 instances of R⁷.

In some embodiments, the compounds of Formula (A) are of Formula (A-VI):

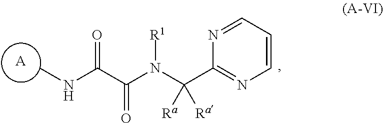

wherein R^{a}, R^{a}′, Ring A and R¹ are as defined in any of the embodiments described herein and the pyrimidinyl is substituted with 0, 1, 2 or 3 instances of R⁷ as defined in any of the embodiments described herein.

In some embodiments, the compounds of Formula (I) are of Formula (VI):

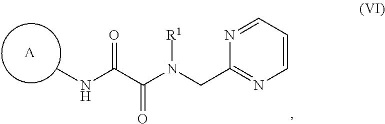

wherein Ring A and R^{I} are as defined in any of the embodiments described herein and the pyrimidinyl is substituted with 0, 1, 2 or 3 instances of R⁷ as defined in any of the embodiments described herein. In some embodiments, the pyrimidinyl is unsubstituted. In some embodiments, the pyrimidinyl is substituted with one instance of R⁷. In some embodiments, the pyrimidinyl is substituted with 2 instances of R⁷. In some embodiments, the pyrimidinyl is substituted with 3 instances of R⁷.

In some embodiments, Ring B is an 8-10 membered bicyclic heteroaryl, wherein the bicyclic heteroaryl is optionally substituted (e.g., substituted with 0, 1, 2 or 3 instances of R⁷ wherein R⁷ is as defined in any of the embodiments described herein). In certain embodiments, Ring B is an 8-10 membered bicyclic heteroaryl (e.g., a 5,5 bicyclic heteroaryl (e.g., 1H-thieno[2,3-c]pyrazolyl, 1H-thieno[3,2-c]pyrazolyl), a 5,6 bicyclic heteroaryl (e.g., indolyl, benzofuranyl, 1H-indazolyl, 2H-indazolyl, benzo[b]thiophenyl, benzo[d]imidazolyl, benzo[d]thiazolyl, benzo[d]oxazoly], [1,2,4]triazolo[4,3-a]pyridinyl, benzo[d]isothiazolyl, imidazo[1,2-a]pyridinyl, imidazo[1,2-a]pyridinyl, 1H-pyrazolo[4,3-b]pyridinyl), 1H-pyrazolo[3,4-b]pyridinyl, thiazolo[5,4-b]pyridinyl), or a 6, 6 bicyclic heteroaryl (e.g., quinolinyl, 1,5-naphthyridinyl, 1,2-dihydro-1,5-naphthyridinyl, 1,2,3,4-tetrahydro-1,8-naphthyridinyl, isoquinolinyl), wherein each bicyclic heteroaryl contains 1, 2, 3 or 4 heteroatoms independently selected from the group consisting of O, N and S, and wherein each bicyclic heteroaryl is optionally substituted (e.g., substituted with 0, 1, 2 or 3 instances of R⁷ wherein R⁷ is as defined in any of the embodiments described herein). In some embodiments, Ring B is a 5,6 bicyclic heteroaryl (e.g., indolyl, benzofuranyl, 1H-indazolyl, 2H-indazolyl, benzo[b]thiophenyl, benzo[d]imidazolyl, benzo[d]thiazolyl, benzo[d]oxazolyl, [1,2,4]triazolo[4,3-a]pyridinyl, benzo[d]isothiazolyl, imidazo[1,2-a]pyridinyl, imidazo[1,2-a]pyridinyl, 1H-pyrazolo[4,3-b]pyridinyl), 1H-pyrazolo[3,4-b]pyridinyl, thiazolo[5,4-b]pyridinyl) or a 6,6 bicyclic heteroaryl (e.g., quinolinyl, isoquinolinyl, 1,5-naphthyridinyl, 1,2-dihydro-1,5-naphthyridinyl, 1,2,3,4-tetrahydro-1,8-naphthyridinyl, isoquinolinyl).

In some embodiments, Ring B is a 5,6 bicyclic heteroaryl (e.g., indolyl, benzofuranyl, 1H-indazolyl, 2H-indazolyl, benzo[b]thiophenyl, benzo[d]imidazolyl, benzo[d]thiazolyl, benzo[d]oxazoly], [1,2,4]triazolo[4,3-a]pyridinyl, benzo[d]isothiazolyl, imidazo[1,2-a]pyridinyl, imidazo[1,2-a]pyridinyl, 1H-pyrazolo[4,3-b]pyridinyl), 1H-pyrazolo[3,4-b]pyridinyl, thiazolo[5,4-b]pyridinyl).

In some embodiments, Ring B is a 6,6 bicyclic heteroaryl (e.g., quinolinyl, isoquinolinyl, 1,5-naphthyridinyl, 1,2-dihydro-1,5-naphthyridinyl, 1,2,3,4-tetrahydro-1,8-naphthyridinyl, isoquinolinyl).

In some embodiments, the bicyclic heteroaryl (e.g., the 5,5 bicyclic heteroaryl, 5,6 bicyclic heteroaryl, 6,6 bicyclic heteroaryl) contains 1, 2 or 3 heteroatoms selected from the group consisting of O, N and S. In some embodiments, the bicyclic heteroaryl contains 1 or 2 heteroatoms selected from the group consisting of O, N and S. In some embodiments, the bicyclic heteroaryl contains 1 heteroatom selected from the group consisting of O, N and S. In some embodiments, the bicyclic heteroaryl contains 2 heteroatoms selected from the group consisting of O, N and S. In some embodiments, the bicyclic heteroaryl contains 3 heteroatoms selected from the group consisting of O, N and S. In some embodiments, the bicyclic heteroaryl contains 4 heteroatoms selected from the group consisting of O, N and S.

In some embodiments, Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl, chromanyl and 1,2,3,4-tetrahydro-1,8-naphthyridinyl, each optionally substituted (e.g., substituted at any available position with 0, 1, 2 or 3 instances of R⁷).

In some embodiments Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazoly], [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each optionally substituted (e.g., substituted with 0, 1, 2 or 3 instances of R⁷).

In certain embodiments, Ring B is selected from the group consisting of 2H-indazolyl, quinolinyl, isoquinolinyl and benzo[d]thiazolyl, each optionally substituted (e.g., substituted with 0, 1, 2 or 3 instances of R⁷). In some embodiments, Ring B is optionally substituted 2H-indazolyl (e.g., substituted with 0, 1, 2 or 3 instances of R⁷). In some embodiments, Ring B is optionally substituted quinolinyl (e.g., substituted with 0, 1, 2 or 3 instances of R⁷). In some embodiments, Ring B is optionally substituted isoquinolinyl (e.g., substituted with 0, 1, 2 or 3 instances of R⁷). In some embodiments, Ring B is optionally substituted benzo[d]thiazolyl (e.g., substituted with 0, 1, 2 or 3 instances of R⁷).

In some embodiments, Ring B is selected from the group consisting of isoquinolinyl and chromanyl, each optionally substituted (e.g., substituted at any available position with 0, 1, 2 or 3 instances of R⁷).

In some embodiments, Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-1-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-y], [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl, chroman-5-yl and thiazolo[5,4-b]pyridin-6-yl, each optionally substituted (e.g., substituted at any available position with 0, 1, 2 or 3 instances of R⁷).

In some embodiments, Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl, each optionally substituted (e.g., substituted with 0, 1, 2 or 3 instances of R⁷).

In some embodiments, Ring B is independently selected from the group consisting of 2H-indazol-6-yl, 2H-indazol-5-yl, quinolin-6-yl, quinolin-7-yl, isoquinolin-6-yl and benzo[d]thiazol-5-yl, each optionally substituted (e.g., substituted with 0, 1, 2 or 3 instances of R⁷). In some embodiments, Ring B is selected from the group consisting of isoquinolin-1-yl and chroman-5-yl, each optionally substituted (e.g., substituted at any available position with 0, 1, 2 or 3 instances of R⁷).

In some embodiments, Ring B is optionally substituted 2H-indazol-6-yl (e.g., substituted with 0, 1, 2 or 3 instances of R⁷). In some embodiments, Ring B is optionally substituted 2H-indazol-5-yl (e.g., substituted with 0, 1, 2 or 3 instances of R⁷). In some embodiments, Ring B is optionally substituted quinolin-6-yl (e.g., substituted with 0, 1, 2 or 3 instances of R⁷). In some embodiments, Ring B is optionally substituted quinolin-7-yl (e.g., substituted with 0, 1, 2 or 3 instances of R⁷). In some embodiments, Ring B is optionally substituted isoquinolin-6-yl (e.g., substituted with 0, 1, 2 or 3 instances of R⁷). In some embodiments, Ring B is optionally substituted benzo[d]thiazol-5-yl (e.g., substituted with 0, 1, 2 or 3 instances of R⁷). In some embodiments, Ring B is isoquinolin-1-yl, optionally substituted (e.g., substituted at any available position with 0, 1, 2 or 3 instances of R⁷). In some embodiments, Ring B is chroman-5-yl, optionally substituted (e.g., substituted at any available position with 0, 1, 2 or 3 instances of R⁷).

In some embodiments Ring B is an 8-10 membered bicyclic heteroaryl selected from the group consisting of:

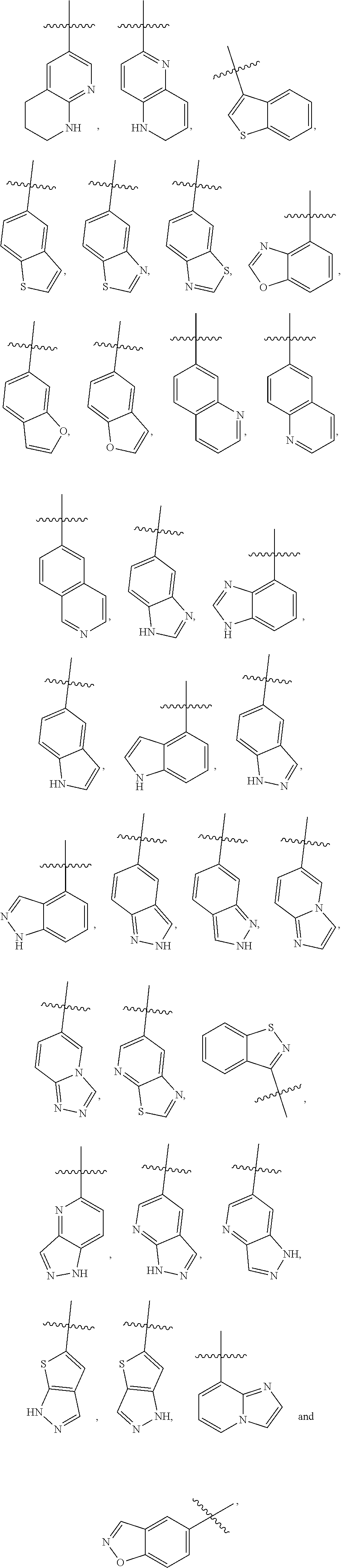

each optionally substituted (e.g., substituted with 0, 1, 2 or 3 instances of R⁷).

As generally defined herein, each R⁷ is independently selected from the group consisting of -D, ═O, —CN, halo, -SFs, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a7}, —N(R^{a7})₂, —C(═O)R^{a7}, —C(═O)OR^{a7}, —NR^{a7}C (═O)R^{a7}, —NR^{a7}C(═O)OR^{a7}, —C(═O)N(R^{a7})₂, —OC(═O)R^{a7}, —OC(═O)N(R^{a7})₂, —S(═O)R^{a7}, —S(═O)₂R^{a7}, —SR^{a7}, —S(═O)(═NR^{a7})R^{a7}, —NR^{a7}S(═O)₂R^{a7} and —S(═O)₂N(R^{a7})₂, wherein each alkyl, cycloalkyl, heterocyclyl, aryl, heteroaryl, cycloalkylalkyl, heterocyclylalkyl, arylalkyl and heteroarylalkyl of R⁷ is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof) wherein R^{a7} is as defined in any of the embodiments described herein.

In some embodiments, each R⁷ is independently selected from the group consisting of -D, ═O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a7}, —N(R^{a7})₂, —C(═O)R^{a7}, —C(═O)OR^{a7}, —NR^{a7}C (═O)R^{a7}, —NR^{a7}C(═O)OR^{a7}, —C(═O)N(R^{a7})₂, —OC(═O)R^{a7}, —OC(═O)N(R^{a7})₂, —S(═O)R^{a7}, —S(═O)₂R^{a7}, —SR^{a7}, —S(═O)(═NR^{a7})R^{a7}, —NR^{a7}S(═O)₂R^{a7} and —S(═O)₂N(R^{a7})₂, wherein each alkyl, cycloalkyl, heterocyclyl, aryl, heteroaryl, cycloalkylalkyl, heterocyclylalkyl, arylalkyl and heteroarylalkyl of R⁷ is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof) wherein R^{a7} is as defined in any of the embodiments described herein.

In one embodiment, each R^{a7} is independently H; —C₁-C₆ alkyl; —C₁-C₆ haloalkyl; —C₁-C₆ heteroalkyl substituted with 0 or 1 instance of ═O; C₃-C₉ cycloalkyl; or 3-10 membered heterocyclyl substituted with 0 or 1 instances of ═O, -Me or a combination thereof.

In some embodiments, each alkyl, cycloalkyl, heterocyclyl, aryl, heteroaryl, cycloalkylalkyl, heterocyclylalkyl, arylalkyl and heteroarylalkyl of R⁷ is unsubstituted. In some embodiments, each alkyl, cycloalkyl, heterocyclyl, aryl, heteroaryl, cycloalkylalkyl, heterocyclylalkyl, arylalkyl and heteroarylalkyl of R⁷ is independently substituted with 1 instance of -Me, —OH, —C(═O)CH₃, —C(═O)NHCH₃, —NH₂ or —NHC(═O)CH₃. In some embodiments, each alkyl, cycloalkyl, heterocyclyl, aryl, heteroaryl, cycloalkylalkyl, heterocyclylalkyl, arylalkyl and heteroarylalkyl of R⁷ is independently substituted with 2 instances of -Me, —OH, —C(═O)CH₃, —C(═O)NHCH₃, —NH₂, —NHC(═O)CH₃ or a combination thereof. In some embodiments, each alkyl, cycloalkyl, heterocyclyl, aryl, heteroaryl, cycloalkylalkyl, heterocyclylalkyl, arylalkyl and heteroarylalkyl of R⁷ is independently substituted with 3 instances of -Me, —OH, —C(═O)CH₃, —C(═O)NHCH₃, —NH₂, —NHC(═O)CH₃ or a combination thereof.

In some embodiments, each R⁷ is independently selected from the group consisting of -D, ═O, —SF₅, halo (e.g., —F, —Cl, —Br), —CN, —C₁-C₆ alkyl (e.g., -Me, -Et, —Pr, -ⁱPr, -sec-Bu, -‘Bu), —C₁-C₆ heteroalkyl (e.g., —CH₂OH, —CH(OH)(CH₃), —C(OH)(CH₃)₂, —CH₂NH₂), -C1-C6 haloalkyl (e.g., —CHF2, —CH₂CF₃, —CF₃, —CF₂CF₃), —C₃-C₉ cycloalkyl (e.g., cyclopropyl, cyclobutyl, cyclopentyl, cyclohexyl), 3-10 membered heterocyclyl (e.g., oxetanyl, pyrolidinyl, piperidinyl, piperazinyl), phenyl, 5-10 membered heteroaryl (e.g., pyrazolyl, thiazolyl, thiophenyl, pyridinyl), cycloalkylalkyl (e.g.—CH₂-cyclopropyl), heterocyclylalkyl (e.g., —CH₂-morpholinyl), heteroarylalkyl (e.g., —CH₂-triazolyl, —CH₂-imidazolyl, —CH₂-pyrazolyl), —OR^{a7} (e.g., —OH, —OCH₃, —O-tetrahydrofuranyl, —O-tetrahydropyran-4-yl, —OCF₃, —OCHF₂), —N(R^{a7})₂, (e.g., —NH₂, —NHR^{a7}, —NHCH₃, —N(CH₃)₂), —NR^{a7}C(═O)R^{a7} (e.g., —NHC(═O)CH₃), —C(═O)N(R^{a7})₂, (e.g., —C(═O)NH₂, —C(═O)NHCH₃), —OC(═O)R^{a7} (e.g., —OC(═O)CH₃), —S(═O)R^{a7} (e.g., —SO₂CH₃), —NR^{a7}S(═O)₂R^{a7} (e.g., —NHSO₂CH₃) and —S(═O)₂N(R^{a7})₂ (e.g., —SO₂NH₂, —SO₂NHCH₃), wherein each alkyl, cycloalkyl, heterocyclyl, phenyl, heteroaryl, cycloalkylalkyl, heterocyclylalkyl, arylalkyl and heteroarylalkyl is optionally substituted (e.g., substituted with 0, 1, 2 or 3 instances of -Me, —OH, —C(═O)CH₃, —NHC(═O)CH₃ or a combination thereof) wherein each R^{a7} is as defined in any of the embodiments described herein. In some embodiments, each R^{a7} is independently selected from the group consisting of H, —C₁-C₆ alkyl, (e.g., -Me, -Et, —Pr, -ⁱPr, -sec-Bu, -^{t}Bu), -C1-C6 haloalkyl (e.g., —CF₃, —CHF2, —CF₂CF₃, —CH₂CF₃), —C₁-C₆ heteroalkyl substituted with 0 or 1 instances of ═O(e.g., —CH₂CH₂N(CH₃)₂, —CH₂C(═O)N(CH₃)₂, —CH(CH₃)CH₂N(CH₃)₂, —CH(CH₃) C(═O)N(CH₃)₂), C₃-C₉ cycloalkyl and 3-10 membered heterocyclyl substituted with 0 or 1 instances of ═O, -Me or a combination thereof (e.g. tetrahydrofuran-3-yl, tetrahydropyran-4-yl, oxetan-3-yl, N—CH₃-2-oxo-pyrrolidin-3-yl).

In some embodiments, each R⁷ is independently selected from the group consisting of -D, ═O, halo (e.g., —F, —Cl, —Br), —CN, —C₁-C₆ alkyl (e.g., -Me, -Et, —Pr, -ⁱPr, -sec-Bu, -‘Bu), —C₁-C₆ heteroalkyl (e.g., —CH₂OH, —CH(OH)(CH₃), —C(OH)(CH₃)₂, —CH₂NH₂), -C1-C6 haloalkyl (e.g., —CHF2, —CH₂CF₃, —CF₃), —C₃-C₉ cycloalkyl (e.g., cyclopropyl, cyclobutyl, cyclopentyl, cyclohexyl), 3-10 membered heterocyclyl (e.g., oxetanyl, pyrolidinyl, piperidinyl, piperazinyl), 5-10 membered heteroaryl (e.g., pyrazolyl, thiazolyl, thiophenyl, pyridinyl), cycloalkylalkyl (e.g.—CH₂-cyclopropyl), heterocyclylalkyl (e.g., -CH₂-morpholinyl), heteroarylalkyl (e.g., —CH₂-triazolyl, —CH₂-imidazolyl, —CH₂-pyrazolyl), —OR^{a7} (e.g., —OH, —OCH₃, —O-tetrahydrofuranyl, —O-tetrahydropyran-4-yl, —OCF₃, —OCHF₂), —N(R^{a7})₂, (e.g., —NH₂, —NHR^{a7}, —NHCH₃, —N(CH₃)₂), —NR^{a7}C(═O)R^{a7} (e.g., —NHC(═O)CH₃), —C(═O)N(R^{a7})₂, (e.g., —C(═O)NH₂, —C(═O)NHCH₃), —OC(═O)R^{a7} (e.g., —OC(═O)CH₃), —S(═O)R^{a7} (e.g., —SO₂CH₃), —NR^{a7}S(═O)₂R^{a7} (e.g., —NHSO₂CH₃) and —S(═O)₂N(R^{a7})₂ (e.g., —SO₂NH₂, —SO₂NHCH₃), wherein each alkyl, cycloalkyl, heterocyclyl, aryl, heteroaryl, cycloalkylalkyl, heterocyclylalkyl, arylalkyl and heteroarylalkyl is optionally substituted (e.g., substituted with 0, 1, 2 or 3 instances of -Me, —OH, —C(═O)CH₃, —NHC(═O)CH₃ or a combination thereof) wherein R^{a7} is as defined in any of the embodiments described herein. In some embodiments, each R^{a7} is independently selected from the group consisting of H, —C₁-C₆ alkyl, (e.g., -Me, -Et, —Pr, -ⁱPr, -sec-Bu, -^{t}Bu), —C₁-C₆ haloalkyl (e.g., —CF₃, —CHF2, —CH₂CF₃), —C₁-C₆ heteroalkyl substituted with 0 or 1 instances of ═O (e.g., —CH₂CH₂N(CH₃)₂, —CH₂C(═O)N(CH₃)₂, —CH(CH₃)CH₂N(CH₃)₂, —CH(CH₃) C(═O)N(CH₃)₂), C₃-C₉ cycloalkyl and 3-10 membered heterocyclyl substituted with 0 or 1 instances of ═O, -Me or a combination thereof (e.g. tetrahydrofuran-3-yl, tetrahydropyran-4-yl, oxetan-3-yl, N—CH₃-2-oxo-pyrrolidin-3-yl).

In some embodiments, each R⁷ is independently selected from the group consisting of -D, halo (e.g., —F, —Cl, Br), —SF5, —CN, —C₁-C₆ alkyl (e.g., -Me, -Et, —Pr, —Pr, -sec-Bu,-^{t}Bu), —C₁-C₆ heteroalkyl (e.g., —CH₂OH, —CH(OH)(CH₃), —C(OH)(CH₃)₂, —CH₂NH₂), -C1-C6 haloalkyl (e.g., —CHF2, —CH₂CF₃, —CF₃), —C₃-C₉ cycloalkyl (e.g., cyclopropyl, cyclobutyl, cyclopentyl, cyclohexyl), phenyl, —OR^{a7} (e.g., —OH, —OCH₃, —O-tetrahydrofuranyl, —O-tetrahydropyran-4-yl, —OCF₃, —OCHF₂), —N(R^{a7})₂, (e.g., —NH₂, —NHR^{a7}, —NHCH₃, —N(CH₃)₂), —NR^{a7}C(═O)R^{a7} (e.g., —NHC(═O)CH₃) and —C(═O)N(R^{a7})₂, (e.g., —C(═O)NH₂, —C(═O)NHCH₃), wherein each alkyl and cycloalkyl is optionally substituted (e.g., substituted with 0, 1, 2 or 3 instances of -Me, —OH, —C(═O)CH₃, —NHC(═O)CH₃ or a combination thereof) wherein R^{a7} is as defined in any of the embodiments described herein. In some embodiments, each R^{a7} is independently selected from the group consisting of H, —C₁-C₆ alkyl, (e.g., -Me, -Et, —Pr, -ⁱPr, -sec-Bu, -^{t}Bu) and —C₁-C₆ haloalkyl (e.g., —CF₃, —CHF2, —CH₂CF₃). In some embodiments, each R^{a7} is independently selected from the group consisting of H and -Me.

In some embodiments, each R⁷ is independently selected from the group consisting of -D, halo (e.g., —F, —Cl, Br), —CN, —C₁-C₆ alkyl (e.g., -Me, -Et, —Pr, -ⁱPr, -sec-Bu, -^{t}Bu), —C₁-C₆ heteroalkyl (e.g., —CH₂OH, —CH(OH)(CH₃), —C(OH)(CH₃)₂, —CH₂NH₂), —C₁-C₆ haloalkyl (e.g., —CHF2, —CH₂CF₃, —CF₃), —C₃-C₉ cycloalkyl (e.g., cyclopropyl, cyclobutyl, cyclopentyl, cyclohexyl), —OR^{a7} (e.g., —OH, —OCH₃, —O-tetrahydrofuranyl, —O-tetrahydropyran-4-yl, —OCF₃, —OCHF₂) and —N(R^{a7})₂, (e.g., —NH₂, —NHR^{a7}, —NHCH₃, —N(CH₃)₂), wherein each alkyl and cycloalkyl is optionally substituted (e.g., substituted with 0, 1, 2 or 3 instances of -Me, —OH, —C(═O)CH₃, —NHC(═O)CH₃ or a combination thereof), wherein R^{a7} is as defined in any of the embodiments described herein. In some embodiments, each R^{a7} independently selected from the group consisting of H, —C₁-C₆ alkyl, (e.g., -Me, -Et, —Pr, -ⁱPr, -sec-Bu, -^{t}Bu) and —C₁-C₆ haloalkyl (e.g., —CF₃, —CHF2, —CH₂CF₃). In some embodiments, each R^{a7} is independently selected from the group consisting of H, -Me, -Et, —Pr,-ⁱPr, -sec-Bu, -^{t}Bu, —CF₃, —CHF₂ and —CH₂CF₃. In some embodiments, each R^{a7} is independently selected from the group consisting of H and -Me.

In some embodiments, each R⁷ is independently selected from the group consisting of -D, halo (e.g., —F, —Cl, Br), —SF5, —CN, —C₁-C₆ alkyl (e.g., -Me, -Et, —Pr, -ⁱPr, -sec-Bu,-^{t}Bu), —C₁-C₆ haloalkyl (e.g., —CHF2, —CH₂CF₃, —CF₃), —N(R^{a7})₂, (e.g., —NH₂, —NHR^{a7}, —NHCH₃, —N(CH₃)₂), —NR^{a7}C (═O)R^{a7} (e.g., —NHC(═O)CH₃) and —C(═O)N(R^{a7})₂, (e.g., —C(═O)NH₂, —C(═O)NHCH₃, wherein each R^{a7} is as defined in any of the embodiments described herein. In some embodiments, each R^{a7} is independently selected from the group consisting of H and —C₁-C₆ alkyl, (e.g., -Me, -Et, —Pr, -ⁱPr, -sec-Bu, -^{t}Bu). In some embodiments, each R^{a7} is independently selected from the group consisting of H, -Me, -Et, —Pr, —Pr, -sec-Bu, -^{t}Bu, —CF₃, —CHF₂ and —CH₂CF₃. In some embodiments, each R^{a7} is independently selected from the group consisting of H and -Me.

In some embodiments, each R⁷ is independently selected from the group consisting of -D, halo (e.g., —F, —Cl, Br), —CN, —C₁-C₆ alkyl (e.g., -Me, -Et, —Pr, -ⁱPr, -sec-Bu, -^{t}Bu) and —C₁-C₆ haloalkyl (e.g., —CHF2, —CH₂CF₃, —CF₃).

In some embodiments, each R⁷ is independently selected from the group consisting of halo (e.g., —F, —Cl, Br), —C₁-C₆ alkyl (e.g., -Me, -Et, —Pr, -ⁱPr, -sec-Bu, -^{t}Bu), and -C1-C6 haloalkyl (e.g., —CHF2, —CH₂CF₃, —CF₂CF₃, —CF₃).

In some embodiments, each R⁷ is independently selected from the group consisting of halo (e.g., —F, —Cl, Br) and —C₁-C₆ haloalkyl (e.g., —CHF2, —CH₂CF₃, —CF₂CF₃, —CF₃).

In some embodiments, each R⁷ is independently selected from the group consisting of -D, —F, —Cl, Br, —CN, —SF5, -Me, -Et, —Pr, -ⁱPr, -sec-Bu, -^{t}Bu, —CHF2, —CH₂CF₃, —CF₂CF₃, —CF₃, —CH₂OH, —CH(OH)(CH₃), —C(OH)(CH₃)₂, —CH₂NH₂, cyclopropyl, cyclobutyl, cyclopentyl, cyclohexyl, phenyl, pyrrolidin-1-yl, piperidin-4-yl, piperazin-4-yl, pyridin-4-yl, pyrazol-1-yl, pyrazol-3-yl, pyrazol-4-yl, pyrazol-5-yl, thiazol-2-yl, thiazol-5-yl, thiophen-2-yl, —CH₂-cyclopropyl, —CH₂-morpholin-4-yl, —CH₂-1,2,4-triazol-1-yl, —CH₂-imidazol-1-yl, —CH₂-pyrazol-1-yl, —OH, —OCH₃, —OCF₃, —OCHF2, —O-tetrahydrofuran-3-yl, —O-tetrahydropyran-4-yl, —O—(N—CH₃-2-oxo-pyrrolidin-3-yl), —OCF₃, —OCHF2, —NH₂, —NHCH₃, —NHCH₂CF₃, —NH-oxetan-3-yl, —NH—(N—CH₃-2-oxo-pyrrolidin-3-yl), —N(CH₃)₂, —NHC(═O)CH₃, —NHCH₂C(═O)N(CH₃)₂, —NHCH(CH₃) C(═O)N(CH₃)₂, —C(═O)NH₂, —C(═O)NHCH₃, —OC(═O)CH₃, —SO₂CH₃, —NHSO₂CH₃, —SO₂NH₂ and —SO₂NHCH₃, wherein each cyclopropyl, cyclobutyl, cyclopentyl, cyclohexyl, phenyl, pyrrolidin-1-yl, piperidin-4-yl, piperazin-4-yl, pyrazol-1-yl, pyrazol-3-yl, pyrazol-4-yl, pyrazol-5-yl, thiazol-2-yl, thiophen-2-yl, —CH₂-cyclopropyl, —CH₂-morpholin-4-yl, —CH₂-1,2,4-triazol-1-yl-CH₂-imidazol-1-yl and —CH₂-pyrazol-1-yl, can be independently substituted with 0, 1, 2 or 3 instances of -Me, —OH, —C(═O)CH₃, —NHC(═O)CH₃ or a combination thereof.

In some embodiments, each R⁷ is independently selected from the group consisting of -D, —F, —Cl, Br, —CN, -Me, -Et, —Pr, -ⁱPr, -sec-Bu, -^{t}Bu, —CHF2, —CH₂CF₃, —CF₂CF₃, —CF₃, —CH₂OH, —CH(OH)(CH₃), —C(OH)(CH₃)₂, —CH₂NH₂, cyclopropyl, cyclobutyl, cyclopentyl, cyclohexyl, pyrrolidin-1-yl, piperidin-4-yl, piperazin-4-yl, pyridin-4-yl, pyrazol-1-yl, pyrazol-3-yl, pyrazol-4-yl, pyrazol-5-yl, thiazol-2-yl, thiazol-5-yl, thiophen-2-yl, —CH₂-cyclopropyl, —CH₂-morpholin-4-yl, —CH₂-1,2,4-triazol-1-yl, —CH₂-imidazol-1-yl, —CH₂-pyrazol-1-yl, —OH, —OCH₃, —OCF₃, —OCHF2, —O-tetrahydrofuran-3-yl, —O-tetrahydropyran-4-yl, —O—(N—CH₃-2-oxo-pyrrolidin-3-yl), —OCF₃, —OCHF2, —NH₂, —NHCH₃, —NHCH₂CF₃, —NH-oxetan-3-yl, —NH—(N—CH₃-2-oxo-pyrrolidin-3-yl), —N(CH₃)₂, —NHC(═O)CH₃, —NHCH₂C(═O)N(CH₃)₂, —NHCH(CH₃) C(═O)N(CH₃)₂, —C(═O)NH₂, —C(═O)NHCH₃, —OC(═O)CH₃, —SO₂CH₃, —NHSO₂CH₃, —SO₂NH₂ and —SO₂NHCH₃, wherein each cyclopropyl, cyclobutyl, cyclopentyl, cyclohexyl, pyrrolidin-1-yl, piperidin-4-yl, piperazin-4-yl, pyrazol-1-yl, pyrazol-3-yl, pyrazol-4-yl, pyrazol-5-yl, thiazol-2-yl, thiophen-2-yl, —CH₂-cyclopropyl, —CH₂-morpholin-4-yl, —CH₂-1,2,4-triazol-1-yl-CH₂-imidazol-1-yl and —CH₂-pyrazol-1-yl, can be independently substituted with 0, 1, 2 or 3 instances of -Me, —OH, —C(═O)CH₃, —NHC(═O)CH₃ or a combination thereof.

In some embodiments, each R⁷ is independently selected from the group consisting of -D, —F, —Cl, Br, —CN, -Me, -Et, —Pr, -ⁱPr, -sec-Bu, -^{t}Bu, —CHF2, —CH₂CF₃, —CF₃, —CH₂OH, —CH(OH)(CH₃), —C(OH)(CH₃)₂, —CH₂NH₂, cyclopropyl, cyclobutyl, cyclopentyl, cyclohexyl, pyrrolidin-1-yl, piperidin-4-yl, piperazin-4-yl, pyridin-4-yl, pyrazol-1-yl, pyrazol-3-yl, pyrazol-4-yl, pyrazol-5-yl, thiazol-2-yl, thiazol-5-yl, thiophen-2-yl, -CH₂-cyclopropyl, —CH₂-morpholin-4-yl, —CH₂-1,2,4-triazol-1-yl, —CH₂-imidazol-1-yl, —CH₂-pyrazol-1-yl, —OH, —OCH₃, —OCF₃, —OCHF2, —O-tetrahydrofuran-3-yl, —O-tetrahydropyran-4-yl, —O—(N—CH₃-2-oxo-pyrrolidin-3-yl), —OCF₃, —OCHF2, —NH₂, —NHCH₃, —NHCH₂CF₃, —NH-oxetan-3-yl, —NH—(N—CH₃-2-oxo-pyrrolidin-3-yl), —N(CH₃)₂, —NHC(═O)CH₃, —NHCH₂C(═O)N(CH₃)₂, —NHCH(CH₃) C(═O)N(CH₃)₂, —C(═O)NH₂, —C(═O)NHCH₃, —OC(═O)CH₃, —SO₂CH₃, —NHSO₂CH₃, —SO₂NH₂ and —SO₂NHCH₃, wherein each cyclopropyl, cyclobutyl, cyclopentyl, cyclohexyl, pyrrolidin-1-yl, piperidin-4-yl, piperazin-4-yl, pyrazol-1-yl, pyrazol-3-yl, pyrazol-4-yl, pyrazol-5-yl, thiazol-2-yl, thiophen-2-yl, —CH₂-cyclopropyl, —CH₂-morpholin-4-yl, —CH₂-1,2,4-triazol-1-yl-CH₂-imidazol-1-yl and —CH₂-pyrazol-1-yl, can be independently substituted with 0, 1, 2 or 3 instances of -Me, —OH, —C(═O)CH₃, —NHC(═O)CH₃ or a combination thereof.

In some embodiments, each R⁷ is independently selected from the group consisting of -D, ═O, —F, —Cl, -Me, -ⁱPr, —CHF2, —CF₂CF₃, —CF₃, —CN, —SF5, cyclopropyl, piperidin-4-yl, piperazin-4-yl, phenyl, pyrazol-1-yl, pyrazol-3-yl, pyrazol-4-yl, pyrazol-5-yl, —OH, —OCH₃, —OCF₃, —OCHF2, —NH₂, —NHCH₃, —N(CH₃)₂, —NHC (O)CH₃, —CONH₂, wherein each cyclopropyl, phenyl, piperazin-4-yl, pyrazol-1-yl, pyrazol-3-yl, pyrazol-4-yl and pyrazol-5-yl, can be independently substituted with 0, 1, 2 or 3 instances of -Me, —OH, —C(═O)CH₃, —NHC(═O)CH₃ or a combination thereof.

In some embodiments, each R⁷ is independently selected from the group consisting of -D, ═O, —F, —Cl, -Me, -ⁱPr, —CHF2, —CF₃, cyclopropyl, piperidin-4-yl, piperazin-4-yl, pyrazol-1-yl, pyrazol-3-yl, pyrazol-4-yl, pyrazol-5-yl, —OH, —OCH₃, —OCF₃, —OCHF2, wherein each cyclopropyl, piperazin-4-yl, pyrazol-1-yl, pyrazol-3-yl, pyrazol-4-yl and pyrazol-5-yl, can be independently substituted with 0, 1, 2 or 3 instances of -Me, —OH, —C(═O)CH₃, —NHC(═O)CH₃ or a combination thereof.

In some embodiments, each R⁷ is independently selected from the group consisting of -D, halo (e.g., —F, —Cl, Br), —CN, —C₁-C₆ alkyl (e.g., -Me, -Et, —Pr, -ⁱPr, -sec-Bu, -^{t}Bu) and —C₁-C₆ haloalkyl (e.g., —CHF2, —CH₂CF₃, —CF₃).

In some embodiments, each R⁷ is independently selected from the group consisting of halo (e.g., —F, —Cl, Br) and —C₁-C₆ haloalkyl (e.g., —CHF2, —CH₂CF₃, —CF₃).

In some embodiments, each R⁷ is independently selected from the group consisting of —F, —Cl, -Me, —Pr, —CF₂CF₃, —CF₃, —CN, —SF₅, phenyl, —NH₂, —NHCH₃, —N(CH₃)₂, —NHC (O)CH₃ and —CONH₂.

In some embodiments, each R⁷ is independently selected from the group consisting of —F, —Cl, -Me, —CF₂CF₃ and —CF₃.

In some embodiments, each R⁷ is independently selected from the group consisting of —F, —Cl, and —CF₃.

In some embodiments, each R⁷ is independently selected from the group consisting of —F and —CF₃.

In some embodiments, R⁷ is H. In some embodiments R⁷ is -D.

In certain embodiments, R⁷ is —C₁-C₆ alkyl. In some embodiments, R⁷ is -Me. In some embodiments, R⁷ is -Et. In some embodiments R⁷ is —Pr or -ⁱPr. In some embodiments R⁷ is -^{t}Bu or -sec-Bu.

In certain embodiments, R⁷ is halo (e.g., fluoro, chloro, bromo, iodo). In some embodiments, R⁷ is —F or —Cl. In some embodiments, R⁷ is —Cl. In some embodiments, R⁷ is —F. In some embodiments, R⁷ is —Br. In some embodiments, R⁷ is —I.

In some embodiments, R⁷ is —CN.

In some embodiments, R⁷ is —C₁-C₆ heteroalkyl. In some embodiments, R⁷ is methoxymethyl (—CH₂OCH₃). In some embodiments, R⁷ is hydroxymethyl (—CH₂OH). In some embodiments, R⁷ is —CH(OH)CH₃, —C(OH)(CH₃)₂. In some embodiments, R⁷ is aminomethyl (e.g., —CH₂NH₂, —CH₂NHCH₃, —CH₂NHCH₂CH₃—CH₂N(CH₃)₂, —CH(CH₃)(N(CH₃)₂), —CH(CH₃)CH₂ (N(CH₃)₂), —CH₂CH₂N(CH₃)₂, —CH₂CH₂CH₂N(CH₃)₂, —CH₂CH₂N(Me)(oxetan-3-yl), —CH(CH₃)N(CH₃)₂, —CH₂C (CH₃)₂N(CH₃)₂, —CH₂CH(CH₃)(N(CH₃)₂). In some embodiments, the heteroalkyl is further substituted with ═O (e.g., —CH₂NHC(═O)CH₃).

In some embodiments, R⁷ is —C₁-C₆ haloalkyl (e.g., —CF₃, —CHF2, —CH₂CF₃, —CF₂CF₃). In some embodiments, R⁷ is trifluoromethyl (—CF₃). In other embodiments, R⁷ is difluoromethyl (—CHF2). In some embodiments R⁷ is trifluoroethyl (—CH₂CF₃). In some embodiments, R⁷ is —CF₂CF₃.

In some embodiments, R⁷ is C₃-C₉ cycloalkyl (e.g., cyclopropyl, cyclobutyl, cyclopentyl, cyclohexyl). In some embodiments, R⁷ is cyclopropyl. In some embodiments, the cyclopropyl is substituted with 1 instance of -Me, —OH, —C(═O)CH₃, —C(═O)NHCH₃, —NH₂, or —NHC(═O)CH₃. In some embodiments, R⁷ is cycloprop-1-yl substituted at the 1 position with 1 instance of -Me, —OH, —C(═O)CH₃, —C(═O)NHCH₃, —NH₂ or —NHC(═O)CH₃. In some embodiments R⁷ is cyclobutyl. In some embodiments, R⁷ is cyclopentyl.

In some embodiments, R⁷ is 3-10 membered heterocyclyl (e.g., oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl, piperazin-2-onyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), each substituted with 0, 1, 2 or 3 instances of -Me, —OH, —C(═O)CH₃, —C(═O)NHCH₃, —NH₂, —NHC(═O)CH₃ or a combination thereof.

In some embodiments, R⁷ is 3-8 membered monocyclic heterocyclyl (e.g., oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl, piperazin-2-onyl) or 5-10 membered bicyclic heterocyclyl(decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl) each substituted with 0, 1, 2 or 3 instances of -Me, —OH, —C(═O)CH₃, —C(═O)NHCH₃, —NH₂, —NHC(═O)CH₃ or a combination thereof.

In some embodiments, R⁷ is 3-8 membered monocyclic heterocyclyl (e.g., oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl, piperazin-2-onyl), each substituted with 0, 1, 2 or 3 instances of -Me, —OH, —C(═O)CH₃, —C(═O)NHCH₃, —NH₂, —NHC(═O)CH₃ or a combination thereof. In some embodiments, R⁷ is selected from the group consisting of oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl and piperazin-2-onyl, each substituted with 0, 1, 2 or 3 instances of -Me, —OH, —C(═O)CH₃, —C(═O)NHCH₃, —NH₂, —NHC(═O)CH₃ or a combination thereof. In some embodiments, R⁷ is oxetanyl. In some embodiments, R⁷ is tetrahydropyranyl. In some embodiments, R⁷ is tetrahydrofuranyl. In some embodiments, R⁷ is azetidinyl. In some embodiments, R⁷ is pyrrolidinyl. In some embodiments, R⁷ is piperidinyl. In some embodiments, R⁷ is tetrahydropyridinyl (e.g., 1,2,3,6 tetrahydropyridinyl). In some embodiments, R⁷ is piperazinyl. In some embodiments, R⁷ is morpholinyl. In some embodiments, R⁷ is azepanyl. In some embodiments, R⁷ is piperidin-2-onyl. In some embodiments, R⁷ is piperazin-2-onyl.

In some embodiments, R⁷ is selected from the group consisting of azetidin-3-yl, tetrahydrofuran-3-yl, tetrahydropyran-4-yl, oxetan-3-yl, morpholin-2-yl, pyrrolidin-1-yl, pyrrolidin-3-yl, piperidin-4-yl, 1,2,3,6 tetrahydropyridin-4-yl, piperidin-3-yl, piperidin-2-one-4-yl, piperazin-4-yl and piperazin-2-on-5-yl. In some embodiments, R⁷ is selected from the group consisting of azetidin-3-yl, tetrahydropyran-4-yl, oxetan-3-yl, morpholin-2-yl, pyrrolidin-3-yl, piperidin-4-yl, piperidin-3-yl, 1,2,3,6 tetrahydropyridin-4-yl, piperidin-2-one-4-yl and piperazin-4-yl.

In some embodiments, R⁷ is tetrahydrofuran-3-yl. In some embodiments, R⁷ is tetrahydropyran-4-yl. In some embodiments, R⁷ is oxetan-3-yl. In some embodiments, R⁷ is morpholin-2-yl. In some embodiments, R⁷ is pyrrolidin-1-yl. In some embodiments, R⁷ is pyrrolidin-3-yl. In some embodiments, R⁷ is piperidin-4-yl. In some embodiments, R⁷ is piperidin-3-yl. In some embodiments, R⁷ is 1,2,3,6 tetrahydropyridin-4-yl. In some embodiments, R⁷ is piperidin-2-one-4-yl. In some embodiments, R⁷ is piperazin-4-yl (e.g., 1-methyl-piperazin-4-yl). In some embodiments, R⁷ is piperazin-2-on-5-yl. In some embodiments, R⁷ is azetidin-3-yl.

In some embodiments, R⁷ is a 5-10 membered bicyclic heterocyclyl (e.g., decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), each substituted with 0, 1, 2 or 3 instances of -Me, —OH, —C(═O)CH₃, —C(═O)NHCH₃, —NH₂, —NHC(═O)CH₃ or a combination thereof.

In some embodiments, the heterocyclyl is substituted with 0, 1, 2 or 3 instances of -D, ═O, -Me, -CD₃, -Et, —C(═O)CH₃ cyclopropyl, oxetan-3-yl, —OH, —N(CH₃)₂, —CH₂N(CH₃)₂ or —C(═O)NHCH₃. In some embodiments, the heterocyclyl is substituted with 0 or 1 instances of -Me.

In some embodiments, R⁷ is monocyclic heterocyclylalkyl (e.g., oxetanylmethyl, aziridinylmethyl, tetrahydrofuranylmethyl, pyrolidinylmethyl, pyrrolidinylethyl, pyrrolidinylpropyl, —CH(CH₃)CH₂-pyrolidinyl, tetrahydropyranylmethyl, piperidinylmethyl, piperazinylmethyl, morpholinylmethyl, azepanylmethyl). In some embodiments, R⁷ is selected from the group consisting of —CH₂-oxetan-3-yl, —CH₂-piperidin-4-yl, —CH₂-pyrrolidin-1-yl, —(CH₂) 2-pyrrolidin-1-yl, —CH(CH₃)CH₂-pyrrolidin-1-yl. In some embodiments, R⁷ is selected from the group consisting of —CH₂-piperidin-4-yl, —CH₂-pyrrolidin-1-yl, —(CH₂) 2-pyrrolidin-1-yl, —CH(CH₃)CH₂-pyrrolidin-1-yl. In some embodiments, R⁷ is selected from the group consisting of —CH₂-piperidin-4-yl, —CH₂-pyrrolidin-1-yl, —(CH₂) 2-pyrrolidin-1-yl, —CH(CH₃)CH₂-pyrrolidin-1-yl.

In some embodiments, the monocyclic heterocyclylalkyl is unsubstituted. In some embodiments, the monocyclic heterocyclylalkyl is optionally substituted (e.g., substituted with 0, 1, 2 or 3 instances of -Me, —OH, —C(═O) Me, —NHC (═O) Me or a combination thereof). In some embodiments, the monocyclic heterocyclylalkyl is substituted with 0, 1 or 2 instances of -Me. In some embodiments R⁷ is cycloalkylalkyl (e.g., cyclopropylmethyl, cyclobutylmethyl, cyclopentylmethyl, cyclohexylmethyl, cycloheptylmethyl). In some embodiments, R⁷ is cyclopropylmethyl.

In some embodiments, R⁷ is a 5-10 membered heteroaryl (e.g., a 5-6 membered monocyclic heteroaryl or an 8-10 membered bicyclic heteroaryl containing 1-3 heteroatoms selected from the group consisting of N, O and S). In some embodiments, R⁷ is a 5-6 membered monocyclic heteroaryl (e.g., a 5-membered monocyclic heteroaryl containing 1-3 heteroatoms selected from the group consisting of O, N and S, a 6-membered monocyclic heteroaryl containing 1-3 N heteroatoms). In some embodiments, R⁷ is a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, R⁷ is a 6-membered monocyclic heteroaryl (e.g., pyridyl, pyrimidinyl, triazinyl, pyrazinyl, pyridazinyl). In some embodiments, the heteroaryl is substituted with 0, 1, 2 or 3 instances of-Me, —OH, —C(═O)CH₃, —C(═O)NHCH₃, —NH₂, —NHC(═O)CH₃ or a combination thereof.

In some embodiments, R⁷ is a 6-10 membered mono or bicyclic aryl. In some embodiments, R⁷ is phenyl. In some embodiments, the phenyl is substituted with 0, 1, 2 or 3 instances of -Me, —OH, —C(═O) Me, —NHC(═O) Me or a combination thereof.

In some embodiments, R⁷ is arylalkyl. In some embodiments, R⁷ is benzyl.

In some embodiments, R⁷ is heteroarylalkyl (e.g., pyridinylmethyl, thiazolylmethyl, triazolylmethyl, pyrazolylmethyl).

In some embodiments, R⁷ is —OR^{a7} wherein R^{a7} is as defined in any of the embodiments described herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy, —O(CH₂)₂N(CH₃)₂, —O-tetrahydrofuran-3-yl, —O-tetrahydropyran-4-yl, —O(N-methyl piperidin-4-yl), —O—(N-Me-2-oxo-pyrrolidin-3-yl), —OCH₂CH(CH₃)N(CH₃)₂, —OCH₂ (N-Methylpyrrolidin-2-yl), —O(N-methyl piperidin-4-yl), —O—(CH₂) 2-pyrrolidin-2-yl, —O—CH₂-piperidin-4-yl, —O—CH₂-oxetan-3-yl). In some embodiments, R⁷ is hydroxy. In some embodiments, R⁷ is methoxy. In some embodiments, R⁷ is difluoromethoxy (—OCHF₂). In some embodiments, R⁷ is trifluoromethoxy (—OCF₃). In some embodiments, R⁷ is ethoxy. In some embodiments, R⁷ is propoxy. In some embodiments, R⁷ is isopropoxy. In some embodiments, R⁷ is cyclopropyloxy. In some embodiments, R′ is cyclobutyloxy. In some embodiments, R⁷ is —O(CH₂)₂N(CH₃)₂.

In some embodiments, R⁷ is hydroxy. In some embodiments, R⁷ is methoxy. In some embodiments, R⁷ is ethoxy. In some embodiments, R⁷ is propoxy. In some embodiments, R⁷ is isopropoxy. In some embodiments R⁷ is difluoromethoxy. (—OCHF₂). In some embodiments, R⁷ is trifluoromethoxy (—OCF₃). In some embodiments, R⁷ is —O(CH₂)₂N(CH₃)₂.

In some embodiments, R⁷ is —N(R^{a7})₂ wherein R^{a7} is as defined in any of the embodiments described herein (e.g., —NH₂, —NHR^{a7}, —N(CH₃)R^{a7}). In some embodiments, R⁷ is —NH₂. In some embodiments, R⁷ is —NHR^{a7} (e.g., —NHCH₃, —NHCH₂CH₃, —NHPr, —NHCH₂CF₃, —NHⁱPr, -NHcyclopropyl, -NHcyclobutyl). In some embodiments, R⁷ is NHCH₂CF₃. In some embodiments, R⁷ is —N(CH₃)R^{a7} (e.g., —N(CH₃)₂, —N(CH₃)CH₂CH₃, —N(CH₃)CH₂CH₂CH₃, —N(CH₃)ⁱPr, —N(CH₃)cyclopropyl, —N(CH₃)cyclobutyl).

In some embodiments, R⁷ is —C(═O)R^{a7} or —C(═O)OR^{a7} wherein R^{a7} is as defined in any of the embodiments described herein. In some embodiments, R⁷ is —C(═O)R^{a7} wherein R^{a7} is as defined in any of the embodiments described herein. In some embodiments, R⁷ is —C(═O)alkyl. In some embodiments, R⁷ is —C(═O)CH₃, —C(═O)cyclopropyl, —C(═O)cyclobutyl, —C(═O)^{t}Bu, —C(═O)ⁱPr, —C(═O)CH₂CH₂CH₃, —C(═O)OCH₃ or —C(═O)CH₂CH₂N(CH₃)₂. In some embodiments, R⁷ is acetyl (—C(═O)CH₃). In some embodiments, R⁷ is —C(═O)CH₂CH₂N(CH₃)₂. In some embodiments, R⁷ is —C(═O)OR^{a7}. In some embodiments, R⁷ is —COOH. In some embodiments, R⁷ is COOCH₃.

In some embodiments, R⁷ is —NR^{a7}C(═O)R^{a7} wherein R^{a7} is as defined in any of the embodiments described herein. In certain embodiments, R⁷ is —NHC(═O)R^{a7} (e.g., —NHC(═O)CH₃, —NHC(═O)CH₂CH₃, —NHC(═O)CH₂CH₂CH₃, —NHC(═O)ⁱPr, —NHC(═O)Bu, —NHC(═O)^{t}Bu, —NHC(═O)Cyclopropyl, —NHC(═O)Cyclobutyl, —C(═O)CH₂CH₂N(CH₃)₂.). In some embodiments, R⁷ is —NHC(═O)CH₃. In some embodiments, R⁷ is —C(═O)CH₂CH₂N(CH₃)₂. In some embodiments, R⁷ is —N(CH₃) C(═O)R^{a7} (e.g., —N(CH₃) C(═O)CH₃, —N(CH₃) C(═O)CH₂CH₃, —N(CH₃) C(═O)CH₂CH₂CH₃, —N(CH₃) C(═O)ⁱPr, —N(CH₃) C(═O)Bu, —N(CH₃) C(═O)^{t}Bu, —N(CH₃) C(═O)Cyclopropyl, —N(CH₃) C(═O)Cyclobutyl).

In some embodiments, R⁷ is —NR^{a7}C(═O)OR^{a7} wherein R^{a7} is as defined in any of the embodiments described herein. In certain embodiments, R⁷ is —NHC(═O)OR^{a7} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O'Bu, —NHC(═O)OCyclopropyl, —NHC(═O)OCyclobutyl). In some embodiments, R⁷ is —N(CH₃) C(═O)OR^{a7} (e.g., —N(CH₃) C(═O)OCH₃, —N(CH₃) C(═O)OCH₂CH₃, —N(CH₃) C(═O)OCH₂CH₂CH₃, —N(CH₃) C(═O)OⁱPr, —N(CH₃) C(═O)OBu, —N(CH₃) C(═O)O'Bu, —N(CH₃) C(═O)OCyclopropyl, —N(CH₃) C(═O)OCyclobutyl).

In some embodiments, R⁷ is —C(═O)N(R^{a7})₂ wherein R^{a7} is as defined in any of the embodiments described herein (e.g., —C(═O)NH₂, —C(═O)NHR^{a7}, —C(═O)N(CH₃)R^{a7}). In some embodiments, R⁷ is —C(═O)NH₂. In certain embodiments, R⁷ is —C(═O)NHR^{a7} (e.g., —C(═O)NHCH₃, —C(═O)NHCH₂CH₃, —C(═O)NHPr, —C(═O)NHⁱPr, —C(═O)NHBu, —C(═O)NH^{t}Bu, —C(═O)NHCyclopropyl, —C(═O)NHCyclobutyl). In some embodiments, R⁷ is —C(═O)NHCH₃. In certain embodiments, R⁷ is —C(═O)N(CH₃)R^{a7} (e.g., —C(═O)N(CH₃)₂, —C(═O)N(CH₃)CH₂CH₃, —C(═O)N(CH₃)CH₂CH₂CH₃, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R⁷ is —OC(═O)N(R^{a7})₂ wherein R^{a7} is as defined in any of the embodiments described herein. In certain embodiments, R⁷ is —OC(═O)NHR^{a7} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁷ is —OC(═O)N(CH₃)R^{a7} (e.g., —OC(═O)N(CH₃)₂, —OC(═O)N(CH₃)CH₂CH₃, —OC(═O)N(CH₃)CH₂CH₂CH₃, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁷ is —OC(═O)R^{a7} wherein R^{a7} is as defined in any of the embodiments described herein. (e.g., —OC(═O)CH₃, —OC(═O)CH₂CH₃, —OC(═O)CH₂CH₂CH₃, —OC(═O)CH₂CH₂CH₃, —OC(═O)Bu, —OC(═O)Bu, —OC(═O)Cyclopropyl, —OC(═O)Cyclobutyl). In some embodiments, R⁷ is —OC(═O)CH₃.

In some embodiments, R⁷ is —S(═O)R^{a7} wherein R^{a7} is as defined in any of the embodiments described herein. In certain embodiments, R⁷ is —S(═O)alkyl (e.g., —S(═O)CH₃, —S(═O)CH₂CH₃, —S(═O)CH₂CH₂CH₃, —S(═O)ⁱPr). In some embodiments R⁷ is —S(═O)CH₃. In certain embodiments, R⁷ is —S(═O)cycloalkyl (e.g., —S(═O)cyclopropyl, —S(═O)cyclobutyl, —S(═O)cyclopentyl, —S(═O)cyclohexyl).

In some embodiments, R⁷ is —S(═O)₂R^{a7} wherein R^{a7} is as defined in any of the embodiments described herein. In certain embodiments, R⁷ is —S(═O)₂alkyl (e.g., —S(═O)₂CH₃, —S(═O)₂CH₂CH₃, —S(═O)₂Pr, —S(═O)₂ⁱ Pr). In some embodiments R⁷ is —S(═O)₂CH₃. In certain embodiments, R⁷ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁷ is S(═O)₂aryl (e.g., —S(═O)₂phenyl).

In some embodiments, R⁷ is —SR^{a7} wherein R^{a7} is as defined in any of the embodiments described herein. In certain embodiments, R⁷ is -Salkyl (e.g., —SCH₃, —SCH₂CH₃, —SPr, —SⁱPr). In certain embodiments, R⁷ is -Scycloalkyl (e.g., -Scyclopropyl, -Scyclobutyl,-Scyclopentyl, -Scyclohexyl). In certain embodiments, R⁷ is -Saryl (e.g., -Sphenyl).

In some embodiments, R⁷ is —SF5.

In some embodiments, R⁷ is —S(═O)(═NR^{a7})R^{a7} wherein R^{a7} is as defined in any of the embodiments described herein. In certain embodiments, R⁷ is —S(═O)(═NH)R^{a7} (e.g., —S(═O)(═NH)CH₃, —S(═O)(═NH)CH₂CH₃, —S(═O)(═NH)CH₂CH₂CH₃, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R⁷ is —S(═O)(═NCH₃)R^{a7} (e.g., —S(═O)(═NCH₃)CH₃, —S(═O)(═NCH₃)CH₂CH₃, —S(═O)(═NCH₃)CH₂CH₂CH₃, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁷ is —NR^{a7}S(═O)₂R^{a7} wherein R^{a7} is as defined in any of the embodiments described herein. In certain embodiments, R⁷ is —NHS(═O)₂alkyl (e.g., —NHS(═O)₂CH₃, —NHS(═O)₂CH₂CH₃, —NHS(═O)₂Pr, —NHS(═O)₂/Pr). In certain embodiments, R⁷ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁷ is —N(CH₃)S(═O)₂alkyl (e.g., —N(CH₃)S(═O)₂CH₃, —N(CH₃)S(═O)₂CH₂CH₃, —N(CH₃)S(═O)₂Pr, —N(CH₃)S(═O)₂ⁱ Pr). In certain embodiments, R⁷ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁷ is —S(═O)₂N(R^{a7})₂ wherein R^{a7} is as defined in any of the embodiments described herein. (e.g., —S(═O)₂NH₂, —S(═O)₂NHR^{a7}, —S(═O)₂N(CH₃)R^{a7}). In some embodiments, R⁷ is —S(═O)₂NH₂. In some embodiments, R⁷ is —S(═O)₂NHR^{a7} (e.g., —S(═O)₂NHCH₃, —S(═O)₂NHCH₂CH₃, —S(═O)₂NHPr, —S(═O)₂NHⁱPr, —S(═O)₂NHcyclopropyl, —S(═O)₂NHcyclobutyl). In some embodiments, R⁷ is —S(═O)₂N(CH₃)R^{a7} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)ⁱPr, —S(═O)₂N(CH₃)cyclopropyl, —S(═O)₂N(CH₃)cyclobutyl).

As generally defined herein, each R^{a1}, R^{a2}, R^{a3}, R^{a4}, R^{a5}, R^{a6} and R^{a7} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is as defined in any of the embodiments described herein).

In some embodiments, each R^{a1}, R^{a2}, R^{a3}, R^{a4}, R^{a5}, R^{a6} and R^{a7} is independently selected from the group consisting of H; —C₁-C₆ alkyl; —C₁-C₆ haloalkyl; —C₁-C₆ heteroalkyl substituted with 0 or 1 instance of ═O; C₃-C₉ cycloalkyl; or 3-10 membered heterocyclyl substituted with 0 or 1 instances of ═O, -Me or a combination thereof.

In some embodiments, each R^{a1}, R^{a2}, R^{a3}, R^{a4}, R^{a5}, R^{a6} and R^{a7} is independently unsubstituted. In some embodiments, each R^{a1}, R^{a2}, R^{a3}, R^{a4}, R^{a5}, R^{a6} and R^{a7} is independently substituted with 1 instance of R⁹. In some embodiments, each R^{a1}, R^{a2}, R^{a3}, R^{a4}, R^{a5}, R^{a6} and R^{a7} is independently substituted with 2 instances of R⁹. In some embodiments, each R^{a1}, R^{a2}, R^{a3}, R^{a4}, R^{a5}, R^{a6} and R^{a7} is independently substituted with 3 instances of R⁹.

In some embodiments, each R^{a1}, R^{a2}, R^{a3}, R^{a4}, R^{a5}, R^{a6} and R^{a7} is independently selected from the group consisting of H, —C₁-C₆ alkyl, —C₁-C₆ haloalkyl, —C₁-C₆ heteroalkyl substituted with 0 or 1 instances of ═O, C₃-C₉ cycloalkyl substituted with 0 or 1 instances of ═O, -Me, —F, —Cl—, —CF₃, and 3-10 membered heterocyclyl substituted with 0 or 1 instances of ═O, -Me, —F, —Cl—, —CF₃ or a combination thereof.

In some embodiments, each R^{a1}, R^{a2}, R^{a3}, R^{a4}, R^{a5}, R^{a6} and R^{a7} is independently selected from the group consisting of H, —C₁-C₆ alkyl, (e.g., -Me, -Et, —Pr, -ⁱPr, -sec-Bu, -‘Bu), —C₁-C₆ haloalkyl (e.g., —CF₃, —CHF2, —CH₂CF₃) and —C₁-C₆ heteroalkyl substituted with 0 or 1 instances of ═O (e.g., —CH₂CH₂N(CH₃)₂, —CH₂C(═O)N(CH₃)₂, —CH(CH₃)CH₂N(CH₃)₂, —CH(CH₃) C(═O)N(CH₃)₂).

In some embodiments, each R^{a1}, R^{a2}, R^{a3}, R^{a4}, R^{a5}, R^{a6} and R^{a7} is independently selected from the group consisting of H, -Me, -Et, —Pr, -ⁱPr, -sec-Bu, -^{t}Bu, —CF₃, —CHF2, —CH₂CF₃ and —CH₂CH₂N(CH₃)₂.

In some embodiments, each R^{a1}, R^{a2}, R^{a3}, R^{a4}, R^{a5}, R^{a6} and R^{a7} is independently selected from the group consisting of H; —C₁-C₆ alkyl; —C₁-C₆ haloalkyl; —C₁-C₆ heteroalkyl substituted with 0 or 1 instance of ═O; C₃-C₉ cycloalkyl; or 3-10 membered heterocyclyl substituted with 0 or 1 instances of ═O, -Me or a combination thereof.

In some embodiments, each R^{a1}, R^{a2}, R^{a3}, R^{a4}, R^{a5}, R^{a6} and R^{a7} is independently selected from the group consisting of H, —C₁-C₆ alkyl, (e.g., -Me, -Et, —Pr, -ⁱPr, -sec-Bu, -“Bu), —C₁-C₆ haloalkyl (e.g., —CF₃, —CHF2, —CH₂CF₃), —C₁-C₆ heteroalkyl substituted with 0 or 1 instances of —O(e.g., —CH₂CH₂N(CH₃)₂, —CH₂C(═O)N(CH₃)₂, —CH(CH₃)CH₂N(CH₃)₂, —CH(CH₃) C(═O)N(CH₃)₂), C₃-C₉ cycloalkyl and 3-10 membered heterocyclyl substituted with 0 or 1 instances of ═O, -Me or a combination thereof (e.g. tetrahydrofuran-3-yl, tetrahydropyran-4-yl, oxetan-3-yl, N—CH₃-2-oxo-pyrrolidin-3-yl).

In some embodiments, each R^{a1}, R^{a2}, R^{a3}, R^{a4}, R^{a5}, R^{a6} and R^{a7} is independently selected from the group consisting of H, —F, —Cl, -Me, -Et, —Pr, -ⁱPr, -sec-Bu, -^{t}Bu, —CF₃, —CHF₂ and —CH₂CF₃.

In some embodiments, each R^{a1}, R^{a2}, R^{a3}, R^{a4}, R^{a5}, R^{a6} and R^{a7} is independently selected from the group consisting of H, —C₁-C₆ alkyl (e.g., -Me, -Et, —Pr, -ⁱPr, -ⁿBu, -^{t}Bu, -sec-Bu,-iso-Bu) and —C₁-C₆ haloalkyl (e.g., —CHF2, —CF₃). In some embodiments, each R^{a1}, R^{a2}, R^{a3}, R^{a4}, R^{a5}, R^{a6} and R^{a7} is independently selected from the group consisting of H and —C₁-C₆ alkyl (e.g., -Me, -Et, —Pr, -ⁱPr, -ⁿBu, -^{t}Bu, -sec-Bu, -iso-Bu).

In some embodiments, each R^{a1}, R^{a2}, R^{a3}, R^{a4}, R^{a5}, R^{a6} and R^{a7} is independently H.

In some embodiments, each R^{a1}, R^{a2}, R^{a3}, R^{a4}, R^{a5}, R^{a6} and R^{a7} is independently-C1-C6 alkyl (e.g., -Me, -Et, —Pr, -ⁱPr, -ⁿBu, -^{t}Bu, -sec-Bu, -iso-Bu). In some embodiments, each R^{a1}, R^{a2}, R^{a3}, R^{a4}, R^{a5}, R^{a6} and R^{a7} is independently-Me. In some embodiments, each R^{a1}, R^{a2}, R^{a3}, R^{a4}, R^{a5}, R^{a6} and R^{a7} is independently-Et. In some embodiments, each R^{a1}, R^{a2}, R^{a3}, R^{a4}, R^{a5}, R^{a6} and R^{a7} is independently-Pr or -ⁱPr.

In some embodiments, each R^{a1}, R^{a2}, R^{a3}, R^{a4}, R^{a5}, R^{a6} and R^{a7} is independently-C1-C6 heteroalkyl. In some embodiments, each R^{a1}, R^{a2}, R^{a3}, R^{a4}, R^{a5}, R^{a6} and R^{a7} is independently methoxymethyl (—CH₂OCH₃). In some embodiments, each R^{a1}, R^{a2}, R^{a3}, R^{a4}, R^{a5}, R^{a6} and R^{a7} is independently hydroxymethyl (—CH₂OH). In some embodiments, each Ral R^{a2}, R^{a3}, R^{a4}, R^{a5}, R^{a6} and R^{a7} is independently aminomethyl (e.g., —CH₂NH₂, —CH₂NHCH₃, —CH₂N(CH₃)₂, —CH₂CH₂N(CH₃)₂). In some embodiments, each R^{a1}, R^{a2}, R^{a3}, R^{a4}, R^{a5}, R^{a6} and R^{a7} is independently-CH₂CH₂N(CH₃)₂).

In some embodiments, each R^{a1}, R^{a2}, R^{a3}, R^{a4}, R^{a5}, R^{a6} and R^{a7} is independently-C1-C6 haloalkyl. In some embodiments, each R^{a1}, R^{a2}, R^{a3}, R^{a4}, R^{a5}, R^{a6} and R^{a7} is independently trifluoromethyl (—CF₃). In other embodiments, each R^{a1}, R^{a2}, R^{a3}, R^{a4}, R^{a5}, R^{a6} and R^{a7} is independently difluoromethyl (—CHF₂).

In some embodiments, each R^{a1}, R^{a2}, R^{a3}, R^{a4}, R^{a5}, R^{a6} and R^{a7} is independently C₃-C₉ cycloalkyl (e.g., cyclopropyl, cyclobutyl, cyclopentyl, cyclohexyl). In some embodiments, each R^{a1}, R^{a2}, R^{a3}, R^{a4}, R^{a5}, R^{a6} and R^{a7} is independently cyclopropyl. In some embodiments each R^{a1}, R^{a2}, R^{a3}, R^{a4}, R^{a5}, R^{a6} and R^{a7} is independently cyclobutyl. In some embodiments, each R^{a1}, R^{a2}, R^{a3}, R^{a4}, R^{a5}, R^{a6} and R^{a7} is independently cyclopentyl. In some embodiments, each R^{a1}, R^{a2}, R^{a3}, R^{a4}, R^{a5}, R^{a6} and R^{a7} is independently cyclohexyl. In some embodiments, the cycloalkyl is substituted with 0, 1, 2 or 3 instances of R⁹, wherein each R⁹ is as defined in any of the embodiments described herein.

In some embodiments, each R^{a1}, R^{a2}, R^{a3}, R^{a4}, R^{a5}, R^{a6} and R^{a7} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, each R^{a1}, R^{a2}, R^{a3}, R^{a4}, R^{a5}, R^{a6} and R^{a7} is independently is oxetanyl. In some embodiments, each R^{a1}, R^{a2}, R^{a3}, R^{a4}, R^{a5}, R^{a6} and R^{a7} is independently tetrahydropyranyl. In some embodiments, each R^{a1}, R^{a2}, R^{a3}, R^{a4}, R^{a5}, R^{a6} and R^{a7} is independently tetrahydrofuranyl. In some embodiments, each R^{a1}, R^{a2}, R^{a3}, R^{a4}, R^{a5}, R^{a6} and R^{a7} is independently azetidinyl. In some embodiments, each R^{a1}, R^{a2}, R^{a3}, R^{a4}, R^{a5}, R^{a6} and R^{a7} is independently pyrrolidinyl. In some embodiments, each R^{a1}, R^{a2}, R^{a3}, R^{a4}, R^{a5}, R^{a6} and R^{a7} is independently piperidinyl. In some embodiments, each R^{a1}, R^{a2}, R^{a3}, R^{a4}, R^{a5}, R^{a6} and R^{a7} is independently piperazinyl. In some embodiments, each R^{a1}, R^{a2}, R^{a3}, R^{a4}, R^{a5}, R^{a6} and R^{a7} is independently morpholinyl. In some embodiments, each R^{a1}, R^{a2}, R^{a3}, R^{a4}, R^{a5}, R^{a6} and R^{a7} is independently azepanyl. In some embodiments, each heterocyclyl is substituted with 0, 1, 2 or 3 instances of R⁹, wherein each R⁹ is as defined in any of the embodiments described herein.

In some embodiments, R^{a1}, R^{a2}, R^{a3}, R^{a4}, R^{a5}, R^{a6} and R^{a7} is independently a 5-10 membered heteroaryl (e.g., a 5-6 membered monocyclic heteroaryl or an 8-10 membered bicyclic heteroaryl containing 1-3 heteroatoms independently selected from the group consisting of N, O and S). In some embodiments, R^{a1}, R^{a2}, R^{a3}, R^{a4}, R^{a5}, R^{a6} and R^{a7} is independently a 5-6 membered monocyclic heteroaryl (e.g., a 5-membered monocyclic heteroaryl containing 1-3 heteroatoms independently selected from the group consisting of O, N and S, a 6-membered monocyclic heteroaryl containing 1-3 N heteroatoms). In some embodiments, R^{a1}, R^{a2}, R^{a3}, R^{a4}, R^{a5}, R^{a6} and R^{a7} is independently a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, R^{a1}, R^{a2}, R^{a3}, R^{a4}, R^{a5}, R^{a6} and R^{a7} is independently thiophenyl (e.g., thiophen-2-yl, thiophen-3-yl). In some embodiments, R^{a1}, R^{a2}, R^{a3}, R^{a4}, R^{a5}, R^{a6} and R^{a7} is independently pyrazolyl (e.g., pyrazol-1-yl, pyrazol-3-yl, pyrazol-5-yl). In some embodiments, R^{a1}, R^{a2}, R^{a3}, R^{a4}, R^{a5}, R^{a6} and R^{a7} is independently thiazolyl (e.g., thiazol-2-yl, thiazol-4-yl, thiazol-5-yl). In some embodiments, R^{a1}, R^{a2}, R^{a3}, R^{a4}, R^{a5}, R^{a6} and R^{a7} is independently a 6-membered monocyclic heteroaryl (e.g., pyridyl, pyrimidinyl, triazinyl, pyrazinyl, pyridazinyl). In some embodiments, R^{a1}, R^{a2}, R^{a3}, R^{a4}, R^{a5}, R^{a6} and R^{a7} is independently pyridinyl (e.g., pyridin-2-yl, pyridin-3-yl, pyridin-4-yl). In some embodiments, R^{a1}, R^{a2}, R^{a3}, R^{a4}, R^{a5}, R^{a6} and R^{a7} is independently pyrimidinyl(e.g, pyrimidin-2-yl, pyrimidin-4-yl, pyrimidin-5-yl). In some embodiments, the heteroaryl is substituted with 0, 1, 2 or 3 instances of R⁹, wherein each R⁹ is as defined in any of the embodiments described herein.

In some embodiments, R^{a1}, R^{a2}, R^{a3}, R^{a4}, R^{a5}, R^{a6} and R^{a7} is independently C₆-C₁₀ aryl. In some embodiments, R^{a1}, R^{a2}, R^{a3}, R^{a4}, R^{a5}, R^{a6} and R^{a7} is independently 6-10 membered mono or bicyclic aryl. In some embodiments, R^{a1}, R^{a2}, R^{a3}, R^{a4}, R^{a5}, R^{a6} and R^{a7} is independently phenyl. In some embodiments, the phenyl is substituted with 0, 1, 2 or 3 instances of R⁹ as defined in any of the embodiments described herein. In some embodiments, the aryl is substituted with 0, 1, 2 or 3 instances of R⁹, wherein each R⁹ is as defined in any of the embodiments described herein.

In some embodiments, each R^{a1}, R^{a2}, R^{a3}, R^{a4}, R^{a5}, R^{a6} and R^{a7} is independently cycloalkylalkyl (e.g., cyclopropylmethyl, cyclobutylmethyl, cyclopentylmethyl, cyclohexylmethyl, cycloheptylmethyl). In some embodiments, the cycloalkylalkyl is substituted with 0, 1, 2 or 3 instances of R⁹, wherein each R⁹ is as defined in any of the embodiments described herein.

In some embodiments, each R^{a1}, R^{a2}, R^{a3}, R^{a4}, R^{a5}, R^{a6} and R^{a7} is independently heterocyclylalkyl (e.g., oxetanylmethyl, aziridinylmethyl, tetrahydrofuranylmethyl, pyrolidinylmethyl, tetrahydropyranylmethyl, piperidinylmethyl, piperazinylmethyl, morpholinylmethyl, azepanylmethyl). In some embodiments, the heterocyclylalkyl is substituted with 0, 1, 2 or 3 instances of R⁹, wherein each R⁹ is as defined in any of the embodiments described herein.

In some embodiments, each R^{a1}, R^{a2}, R^{a3}, R^{a4}, R^{a5}, R^{a6} and R^{a7} is independently arylalkyl. In some embodiments, each R^{a1}, R^{a2}, R^{a3}, R^{a4}, R^{a5}, R^{a6} and R^{a7} is independently benzyl. In some embodiments, the arylalkyl is substituted with 0, 1, 2 or 3 instances of R⁹, wherein each R⁹ is as defined in any of the embodiments described herein.

In some embodiments, each R^{a1}, R^{a2}, R^{a3}, R^{a4}, R^{a5}, R^{a6} and R^{a7} is independently heteroarylalkyl (e.g., pyridinylmethyl, thiazolylmethyl, triazolylmethyl, pyrazolylmethyl). In some embodiments, the heteroarylalkyl is substituted with 0, 1, 2 or 3 instances of R⁹, wherein each R⁹ is as defined in any of the embodiments described herein.

As generally defined herein, each R⁸ is independently selected from the group consisting of halo, ═O, —CN, —OH, —NH₂, —C₁-C₆ alkyl, —C₁-C₆ haloalkyl, —O(C₁-C₆ alkyl), —O(C₁-C₆ haloalkyl), —NH(C₁-C₆ alkyl), —NH(C₁-C₆ haloalkyl), —N(C₁-C₆ alkyl)₂, —N(C₁-C₆ haloalkyl)₂, —C(O)NH₂, —NHC (O)(C₁-C₆ alkyl), C₃-C₉ cycloalkyl and C₁-C₆ heteroalkyl.

In some embodiments, each R⁸ is independently selected from the group consisting of halo, ═O, —CN, —OH, —NH₂, —C₁-C₆ alkyl, —C₁-C₆ haloalkyl, —O(C₁-C₆ alkyl), —O(C₁-C₆ haloalkyl), —NH(C₁-C₆ alkyl), —NH(C₁-C₆ haloalkyl), —N(C₁-C₆ alkyl)₂, —N(C₁-C₆ haloalkyl)₂, C₃-C₉ cycloalkyl and C₁-C₆ heteroalkyl.

In some embodiments, R⁸ is independently selected from the group consisting of halo, ═O, —CN, —OH, —NH₂, —C₁-C₆ alkyl, —O(C₁-C₆ alkyl), —O(C₁-C₆ haloalkyl), —NH(C₁-C₆ alkyl), —NH(C₁-C₆ haloalkyl), —N(C₁-C₆ alkyl)₂, —N(C₁-C₆ haloalkyl)₂, C₃-C₉ cycloalkyl and C₁-C₆ heteroalkyl.

In some embodiments, each R⁸ is independently selected from the group consisting of halo (e.g., —F, —Cl), ═O, —CN, —OH, —NH₂, —C₁-C₆ alkyl (e.g., -Me, -Et-, —Pr, -ⁱPr, -sec-Bu,-^{t}Bu), —C₁-C₆ haloalkyl (e.g., —CF₃), —O(C₁-C₆ alkyl)(e.g., —OCH₃, —OCH₂CH₃, —OCH(CH₃)₂), —O(C₁-C₆ haloalkyl)(e.g., —OCF₃, —OCHF2, —OCH₂CF₃), C₃-C₉ cycloalkyl (e.g., cyclopropyl, cyclobutyl, cyclopentyl, cyclohexyl) and C₁-C₆ heteroalkyl (e.g., -CH₂OCH₃, —CH₂CH₂OCH₃, —CH₂NHCH₃, —CH₂CH₂NHCH₃, —CH₂N(CH₃)₂, —CH₂CH₂N(CH₃)₂).

In some embodiments, each R⁸ is independently selected from the group consisting of halo (e.g., —F, —CI), —O, —CN, —OH, —NH₂, —C₁-C₆ alkyl (e.g., -Me, -Et-, —Pr, -ⁱPr, -sec-Bu,-^{t}Bu), —O(C₁-C₆ alkyl)(e.g., —OCH₃, —OCH₂CH₃, —OCH(CH₃)₂), —O(C₁-C₆ haloalkyl) (e.g., —OCF₃, —OCHF2, —OCH₂CF₃), C₃-C₉ cycloalkyl (e.g., cyclopropyl, cyclobutyl, cyclopentyl, cyclohexyl) and C₁-C₆ heteroalkyl (e.g., —CH₂OCH₃, —CH₂CH₂OCH₃, —CH₂NHCH₃, —CH₂CH₂NHCH₃, —CH₂N(CH₃)₂, —CH₂CH₂N(CH₃)₂).

In some embodiments, each R⁸ is independently selected from the group consisting of halo (e.g., —F, —Cl) and —C₁-C₆ alkyl (e.g., -Me, -Et-, —Pr, -ⁱPr, -sec-Bu, -^{t}Bu).

In some embodiments, each R⁸ is independently selected from the group consisting of —F, —Cl, —O, —CN, —OH, —NH₂, —NHCH₃, —N(CH₃)₂, —NHC (O)CH₃, —CONH₂, -Me, -Et-, —Pr, -ⁱPr, -sec-Bu, -^{t}Bu, —CF₃, —OCH₃, —OCH₂CH₃, —OCH(CH₃)₂, —OCF₃, —OCHF2, —OCH₂CF₃, cyclopropyl, cyclobutyl, cyclopentyl, cyclohexyl, —CH₂OCH₃, —CH₂CH₂OCH₃, —CH₂NHCH₃, —CH₂CH₂NHCH₃, —CH₂N(CH₃)₂ and —CH₂CH₂N(CH₃)₂.

In some embodiments, each R⁸ is independently selected from the group consisting of —F, —Cl, ═O, —CN, —OH, —NH₂, -Me, -Et-, —Pr, —Pr, -sec-Bu, -^{t}Bu, —CF₃, —OCH₃, —OCH₂CH₃, —OCH(CH₃)₂, —OCF₃, —OCHF2, —OCH₂CF₃, cyclopropyl, cyclobutyl, cyclopentyl, cyclohexyl, —CH₂OCH₃, —CH₂CH₂OCH₃, —CH₂NHCH₃, —CH₂CH₂NHCH₃, —CH₂N(CH₃)₂ and —CH₂CH₂N(CH₃)₂.

In some embodiments, each R⁸ is independently selected from the group consisting of —F, —Cl, —O, —CN, —OH, —NH₂, -Me, -Et-, —Pr, -ⁱPr, -sec-Bu, -^{t}Bu, —OCH₃, —OCH₂CH₃, —OCH(CH₃)₂, —OCF₃, —OCHF2, —OCH₂CF₃, cyclopropyl, cyclobutyl, cyclopentyl, cyclohexyl, —CH₂OCH₃, —CH₂CH₂OCH₃, —CH₂NHCH₃, —CH₂CH₂NHCH₃, —CH₂N(CH₃)₂ and —CH₂CH₂N(CH₃)₂.

In some embodiments, each R⁸ is independently selected from the group consisting of —F, —Cl, -Me, —NH₂, —NHCH₃, —N(CH₃)₂, —NHC (O)CH₃, —C(O)NH₂, —CF₃, —OCHF2, -cyclopropyl, and —CH₂OCH₃.

In some embodiments, each R⁸ is independently selected from the group consisting of —F, -Me, —CF₃, —OCHF2, -cyclopropyl, and —CH₂OCH₃.

In some embodiments, each R⁸ is independently selected from the group consisting of —F, -Me, —OCHF2, -cyclopropyl and —CH₂OCH₃.

In some embodiments, each R⁸ is independently selected from the group consisting of -Me and —OCHF₂.

In some embodiments, each R⁸ is independently selected from the group consisting of -Me, —F, —Cl and —CF₃.

In some embodiments, each R⁸ is independently halo (e.g., fluoro, chloro, bromo, iodo). In some embodiments, R⁸ is —Cl. In some embodiments, R⁸ is —F. In some embodiments, R⁸ is —Br. In some embodiments, R⁸ is —I.

In some embodiments, each R⁸ is independently-CN.

In some embodiments, each R⁸ is independently halo ═O.

In some embodiments, each R⁸ is independently-OH.

In some embodiments, each R⁸ is independently-NH2.

In some embodiments, each R⁸ is independently-C₁-C₆ haloalkyl. In some embodiments, R⁸ is —CF₃.

In some embodiments, each R⁸ is independently-C₁-C₆ alkyl. In some embodiments, R⁸ is -Me. In some embodiments, R⁸ is -Et. In some embodiments R8 is —Pr or-iPr.

In some embodiments, each R⁸ is independently-C₁-C₆ heteroalkyl. In some embodiments, R⁸ is methoxymethyl (—CH₂OCH₃). In some embodiments, R⁸ is hydroxymethyl (—CH₂OH). In some embodiments, R⁸ is aminomethyl (e.g., —CH₂NH₂, —CH₂NHCH₃, —CH₂N(CH₃)₂).

In some embodiments, each R⁸ is independently-O(C₁-C₆ alkyl)(e.g., methoxy, ethoxy, propoxy, isopropoxy, butoxy, tertbutoxy). In some embodiments, R⁸ is methoxy. In some embodiments, R⁸ is ethoxy. In some embodiments, R⁸ is propoxy. In some embodiments, R⁸ is isopropoxy.

In some embodiments, each R⁸ is independently-O(C₁-C₆ haloalkyl). In some embodiments, R⁸ is trifluoromethoxy (—OCF₃), In other embodiments, R⁸ is difluoromethoxy (—OCHF₂).

In some embodiments each R⁸ is independently-NH(C₁-C₆ alkyl)(e.g., —NHCH₃, —NHCH₂CH₃, —NHPr, —NHⁱPr).

In some embodiments each R⁸ is independently-N(C₁-C₆ alkyl)₂ (e.g., —N(CH₃)₂, —N(CH₃)CH₂CH₃, —N(CH₃)CH₂CH₂CH₃, —N(CH₃)ⁱPr).

In some embodiments each R⁸ is independently-NH(C₁-C₆ haloalkyl)(e.g., —NHCH₂CF₃).

In some embodiments each R⁸ is independently-N(C₁-C₆ haloalkyl)₂ (e.g., —N(CH₂CF₃)₂).

In some embodiments, each R⁸ is independently C₃-C₉ cycloalkyl (e.g., cyclopropyl, cyclobutyl, cyclopentyl, cyclohexyl). In some embodiments, R⁸ is cyclopropyl. In some embodiments R8 is cyclobutyl. In some embodiments, R⁸ is cyclopentyl. In some embodiments, R⁸ is cyclohexyl.

As generally defined herein, each R⁹ is independently selected from the group consisting of ═O, halo, —CN, —C₁-C₆ alkyl, —C₁-C₆ heteroalkyl, —C₁-C₆ hydroxyalkyl, -C1-C6 haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, -NRDC(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu, -iso-Bu).and C₃-C₉ cycloalkyl (e.g., cyclopropyl, cyclobutyl, cyclopentyl, cyclohexyl). In some embodiments, each R^{b} is H or -Me.

In some embodiments, each R⁹ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, -NRDC(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu)), and C₃-C₉ cycloalkyl (e.g., cyclopropyl, cyclobutyl, cyclopentyl, cyclohexyl). In some embodiments, each R^{b} is H or -Me.

In some embodiments, each R⁹ is independently selected from the group consisting of ═O, halo (e.g., —F, —Cl), —CN, —C₁-C₆ alkyl (e.g., -Me, -Et, iPr), —C₁-C₆ heteroalkyl, -C1-C6 hydroxyalkyl (e.g., —CH₂OH), —C₁-C₆ haloalkyl (e.g., —CF₃, —CH₂CF₃, —CF₂CH₃, —CHF2, —CH₂F), 3-10 membered heterocyclyl, —N(R^{b})₂ (e.g., —N(CH₃)₂), —OH and —OC₁—C₆ alkyl (e.g., —OCH₃).

In some embodiments, each R⁹ is independently selected from the group consisting of halo (e.g., —F, —Cl), —CN, —C₁-C₆ haloalkyl (e.g., —CF₃, —CH₂CF₃, —CF₂CH₃, —CHF2, —CH₂F), —N(R^{b})₂ (e.g., —N(CH₃)₂), and —OH.

In some embodiments, each R⁹ is independently selected from the group consisting of halo (e.g., —F, —Cl), —CN, —C₁-C₆ alkyl (e.g., -Me, -Et, iPr), —C₁-C₆ hydroxyalkyl (e.g., —CH₂OH), —C₁-C₆ haloalkyl (e.g., —CF₃, —CH₂CF₃, —CF₂CH₃, —CHF2, —CH₂F) and —OH.

In some embodiments, each R⁹ is independently selected from the group consisting of F, —CN, -Me, -Et, —CH₂OH, —OH, —CF₃, —CH₂CF₃, —CF₂CH₃, —CHF2, —CH₂F and —N(CH₃)₂.

In some embodiments, each R⁹ is independently selected from the group consisting of -Me, —CF₃ and —N(CH₃)₂.

In some embodiments, each R⁹ is independently selected from the group consisting of F, —Cl, —CN, -Me, -Et, iPr, —CH₂OH, —CF₃, —CH₂CF₃, —CF₂CH₃, —CHF2, —CH₂F, —OH, —OCH₃ and —N(CH₃)₂.

In some embodiments, R⁹ is -Me. In some embodiments, R⁹ is —CF₃. In some embodiments, R⁹ is —N(CH₃)₂. In some embodiments, R⁹ is —F. In some embodiments, R⁹ is —OH. In some embodiments, R⁹ is —OCH₃.

In one embodiment, provided is a compound selected from the group consisting of the compounds of Table 1, or pharmaceutically acceptable salts thereof.

Compounds described herein (e.g., a compound of Formula (A) to (A-VI) or a compound of Table 1, or pharmaceutically acceptable salts thereof) are useful as inhibitors of PRMT5 (e.g., MTA uncompetitive PRMT5 inhibitors).

Table 1 indicates IC₅₀ and IC₉₀ values in an MTAP-isogenic cell line pair for exemplary compounds in an SDMA in-cell western assay (described in Example 171) (columns 4-6). HAP1 MTAP-intact is a cell line in which endogenous levels of MTAP are expressed, and HAP1 MTAP-deleted is an MTAP-null cell line. For Table 1, “a” and “aa” indicates an IC₅₀ of <5 nM, “b” and “bb” indicates an IC₅₀ equal to or greater than 5 nM but less than 50 nM, and “c” and “cc” indicates an IC₅₀ of greater than or equal to 50 nM in the HAP1 MTAP-intact (column 4) and the HAP1 MTAP-deleted (column 5) assays, respectively. Similarly, “aaa” indicates an IC₉₀ of <75 nM, “bbb” indicates an IC₉₀ equal to or greater than 75 nM but less than 125 nM, and “ccc” indicates an IC₉₀ of greater than or equal to 125 nM in the HAP1 MTAP-deleted (column 6) assay.

In column 7, “A” indicates an IC₅₀ ratio greater than or equal to 30 fold between the IC₅₀ in the HAP1 MTAP-intact cell line and the HAP1 MTAP-deleted cell line; “B” indicates an IC₅₀ ratio greater than or equal to 15 fold but lower than 30 fold between the IC₅₀ in the HAP1 MTAP-intact cell line and the HAP1 MTAP-deleted cell line; “C” indicates an IC 50 ratio of less than 15 fold between the IC₅₀ in the HAP1 MTAP-intact cell line and the HAP1 MTAP-deleted cell line. Compounds with a ratio in the SDMA in-cell western assay of equal to or greater than 3 fold are considered MTAP-selective.

Table 1 additionally indicates IC₅₀ values in a viability assay for the MTAP-deleted cell line (described in Example 172)(column 8), indicating the effect of treatment with compound on cell survival. In column 10, a value of A* indicates an IC₅₀ of less than 100 nM, a value of B* indicates an IC₅₀ equal to or greater than 100 nM but less than 1 μM, and a value of C* indicates an IC₅₀ greater than or equal to 1 μM.

Unless otherwise indicated, the absolute stereochemistry of all chiral atoms is as depicted. Compounds marked with (or) or (rel) are single enantiomers wherein the absolute stereochemistry was arbitrarily assigned (e.g., based on chiral SFC elution as described in the Examples section). Compounds marked with (and) or (rac) are mixtures of enantiomers wherein the relative stereochemistry is as shown. Compounds that have a stereogenic center where the configuration is not indicated in the structure as depicted and that are not marked in the “stereochemistry” column are mixtures of enantiomers. Compounds marked with (abs) are single enantiomers wherein the absolute stereochemistry is as indicated. In some instances, different indicators selected from (abs)(or) and (and) apply to different portions of the molecule.

A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

TABLE 1
Exemplary compounds and biological data

| | | | | | | Hap1 | Hap1 |
|---|---|---|---|---|---|---|---|
| | | | Hap1 | Hap1 | Hap1 | MTAP | MTAP |
| | | | MTAP | MTAP | MTAP | intact/ | deleted |
| | | Stereo- | intact | deleted | deleted | deleted | viability |
| STRUCTURE | Nr | chemistry | IC50 | IC50 | IC90 | ratio | IC50 |
| 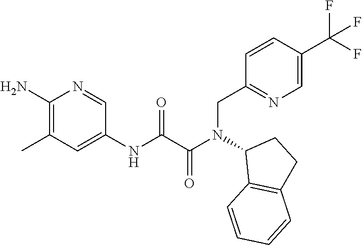 [SMILES: Cc1cc(NC(=O)C(=O)N(Cc2ccc(C(F)(F)F)cn2)[C@@H]2CCc3ccccc32)cnc1N] | 1 | (abs) | b | aa | aaa | C | A* |
| 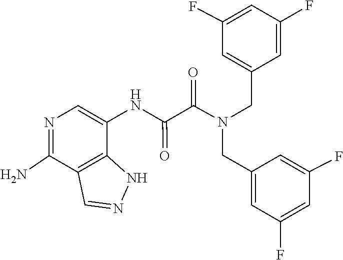 [SMILES: Nc1ncc(NC(=O)C(=O)N(Cc2cc(F)cc(F)c2)Cc2cc(F)cc(F)c2)c2[nH]ncc12] | 2 | | c | bb | ccc | A | B* |
| 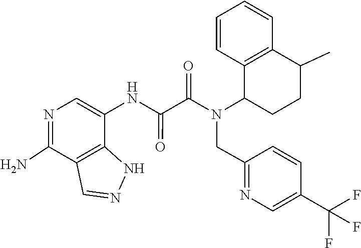 [SMILES: CC1CCC(N(Cc2ccc(C(F)(F)F)cn2)C(=O)C(=O)Nc2cnc(N)c3cn[nH]c23)c2ccccc21] | 3 | | b | bb | aaa | C | A* |
| 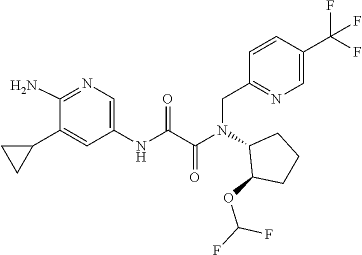 [SMILES: Nc1ncc(NC(=O)C(=O)N(Cc2ccc(C(F)(F)F)cn2)[C@@H]2CCC[C@H]2OC(F)F)cc1C1CC1] | 4 | (abs) | c | bb | bbb | A | B* |
| 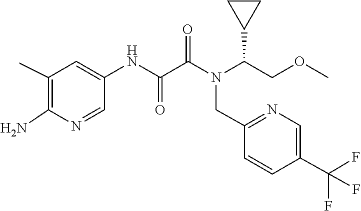 [SMILES: COC[C@@H](C1CC1)N(Cc1ccc(C(F)(F)F)cn1)C(=O)C(=O)Nc1cnc(N)c(C)c1] | 5 | (abs) | b | bb | aaa | C | A* |
| 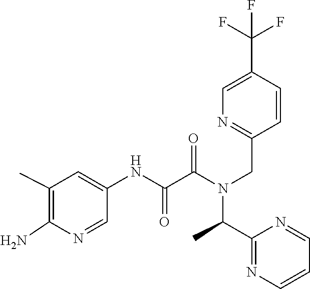 [SMILES: Cc1cc(NC(=O)C(=O)N(Cc2ccc(C(F)(F)F)cn2)[C@H](C)c2ncccn2)cnc1N] | 6 | (abs) | a | aa | ccc | C | B* |
| 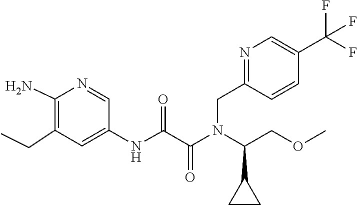 [SMILES: CCc1cc(NC(=O)C(=O)N(Cc2ccc(C(F)(F)F)cn2)[C@@H](COC)C2CC2)cnc1N] | 7 | (abs) | c | bb | ccc | B | B* |
| 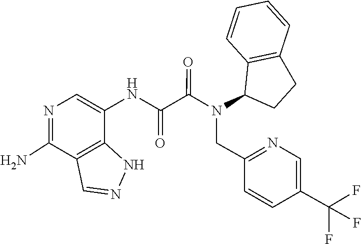 [SMILES: Nc1ncc(NC(=O)C(=O)N(Cc2ccc(C(F)(F)F)cn2)[C@@H]2CCc3ccccc32)c2[nH]ncc12] | 8 | (abs) | a | aa | aaa | C | A* |
| 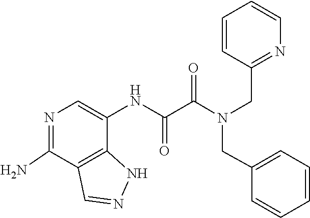 [SMILES: Nc1ncc(NC(=O)C(=O)N(Cc2ccccc2)Cc2ccccn2)c2[nH]ncc12] | 9 | | c | aa | aaa | A | A* |
| 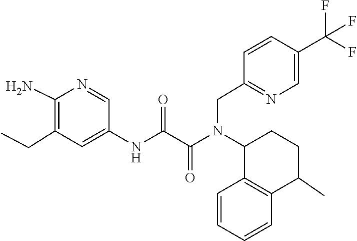 [SMILES: CCc1cc(NC(=O)C(=O)N(Cc2ccc(C(F)(F)F)cn2)C2CCC(C)c3ccccc32)cnc1N] | 10 | | c | bb | bbb | C | B* |
| 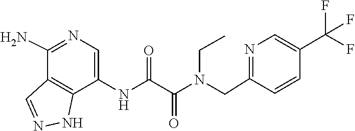 [SMILES: CCN(Cc1ccc(C(F)(F)F)cn1)C(=O)C(=O)Nc1cnc(N)c2cn[nH]c12] | 11 | | C | bb | aaa | A | A* |
| 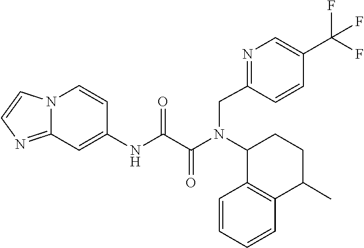 [SMILES: CC1CCC(N(Cc2ccc(C(F)(F)F)cn2)C(=O)C(=O)Nc2ccn3ccnc3c2)c2ccccc21] | 12 | | c | bb | ccc | C | B* |
| 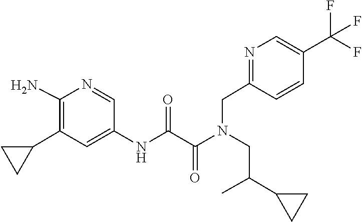 [SMILES: CC(CN(Cc1ccc(C(F)(F)F)cn1)C(=O)C(=O)Nc1cnc(N)c(C2CC2)c1)C1CC1] | 13 | | C | bb | ccc | A | B* |
| 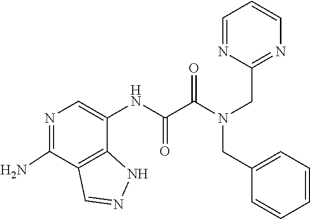 [SMILES: Nc1ncc(NC(=O)C(=O)N(Cc2ccccc2)Cc2ncccn2)c2[nH]ncc12] | 14 | | C | aa | bbb | A | A* |
| 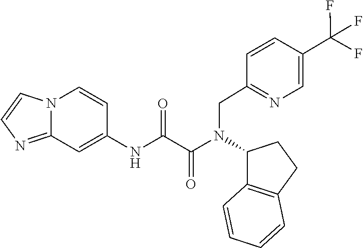 [SMILES: O=C(Nc1ccn2ccnc2c1)C(=O)N(Cc1ccc(C(F)(F)F)cn1)[C@@H]1CCc2ccccc21] | 15 | (abs) | c | bb | ccc | A | B* |
| 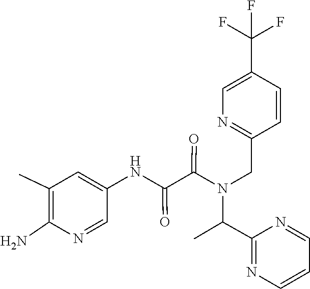 [SMILES: Cc1cc(NC(=O)C(=O)N(Cc2ccc(C(F)(F)F)cn2)C(C)c2ncccn2)cnc1N] | 16 | | b | bb | ccc | C | B* |
| 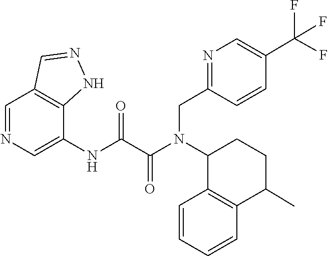 [SMILES: CC1CCC(N(Cc2ccc(C(F)(F)F)cn2)C(=O)C(=O)Nc2cncc3cn[nH]c23)c2ccccc21] | 17 | | c | bb | ccc | C | A* |
| 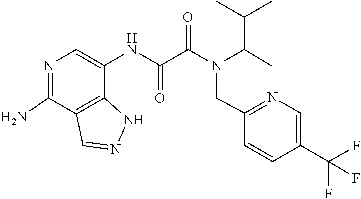 [SMILES: CC(C)C(C)N(Cc1ccc(C(F)(F)F)cn1)C(=O)C(=O)Nc1cnc(N)c2cn[nH]c12] | 18 | (abs) | b | aa | aaa | C | A* |
| 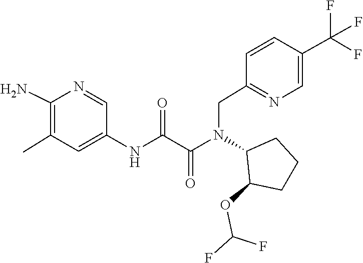 [SMILES: Cc1cc(NC(=O)C(=O)N(Cc2ccc(C(F)(F)F)cn2)[C@@H]2CCC[C@H]2OC(F)F)cnc1N] | 19 | (abs) | b | aa | ccc | C | B* |
| 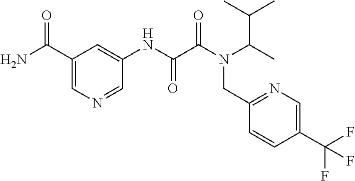 [SMILES: CC(C)C(C)N(Cc1ccc(C(F)(F)F)cn1)C(=O)C(=O)Nc1cncc(C(N)=O)c1] | 20 | (abs) | c | bb | ccc | B | B* |
| 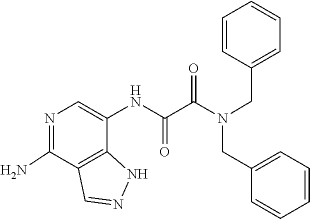 [SMILES: Nc1ncc(NC(=O)C(=O)N(Cc2ccccc2)Cc2ccccc2)c2[nH]ncc12] | 21 | | c | aa | aaa | A | A* |
| 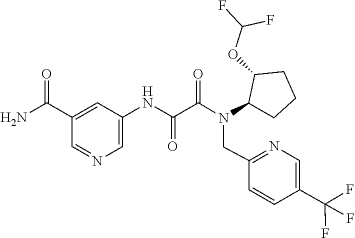 [SMILES: NC(=O)c1cncc(NC(=O)C(=O)N(Cc2ccc(C(F)(F)F)cn2)[C@@H]2CCC[C@H]2OC(F)F)c1] | 22 | (abs) | c | bb | bbb | A | B* |
| 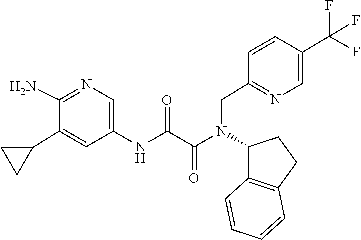 [SMILES: Nc1ncc(NC(=O)C(=O)N(Cc2ccc(C(F)(F)F)cn2)[C@@H]2CCc3ccccc32)cc1C1CC1] | 23 | (abs) | c | bb | bbb | A | A* |
| 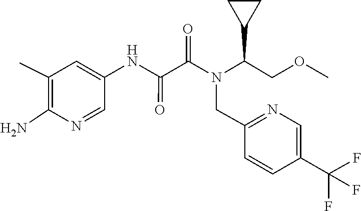 [SMILES: COC[C@H](C1CC1)N(Cc1ccc(C(F)(F)F)cn1)C(=O)C(=O)Nc1cnc(N)c(C)c1] | 24 | (abs) | b | bb | ccc | C | A* |
| 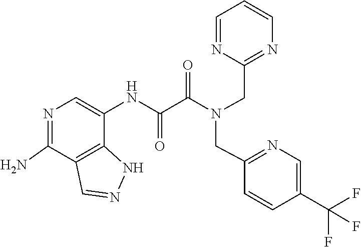 [SMILES: Nc1ncc(NC(=O)C(=O)N(Cc2ccc(C(F)(F)F)cn2)Cc2ncccn2)c2[nH]ncc12] | 25 | | b | aa | aaa | B | A* |
| 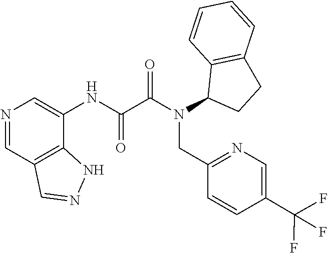 [SMILES: O=C(Nc1cncc2cn[nH]c12)C(=O)N(Cc1ccc(C(F)(F)F)cn1)[C@@H]1CCc2ccccc21] | 26 | | c | aa | bbb | B | B* |
| 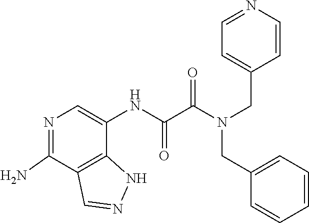 [SMILES: Nc1ncc(NC(=O)C(=O)N(Cc2ccccc2)Cc2ccncc2)c2[nH]ncc12] | 27 | | c | aa | aaa | A | A* |
| 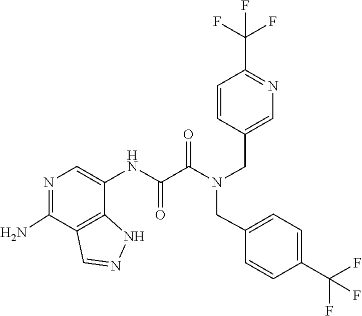 [SMILES: Nc1ncc(NC(=O)C(=O)N(Cc2ccc(C(F)(F)F)cc2)Cc2ccc(C(F)(F)F)nc2)c2[nH]ncc12] | 28 | | c | aa | ccc | A | B* |
| 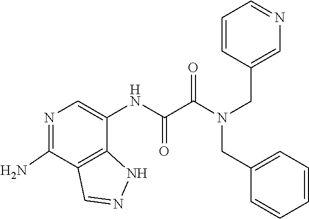 [SMILES: Nc1ncc(NC(=O)C(=O)N(Cc2ccccc2)Cc2cccnc2)c2[nH]ncc12] | 29 | | c | aa | aaa | A | A* |
| 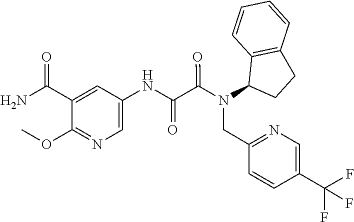 [SMILES: COc1ncc(NC(=O)C(=O)N(Cc2ccc(C(F)(F)F)cn2)[C@@H]2CCc3ccccc32)cc1C(N)=O] | 30 | (abs) | c | aa | aaa | A | A* |
| 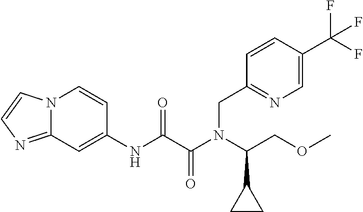 [SMILES: COC[C@@H](C1CC1)N(Cc1ccc(C(F)(F)F)cn1)C(=O)C(=O)Nc1ccn2ccnc2c1] | 31 | (abs) | c | bb | ccc | B | B* |
| 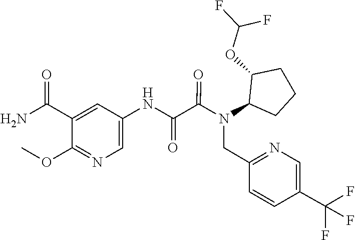 [SMILES: COc1ncc(NC(=O)C(=O)N(Cc2ccc(C(F)(F)F)cn2)[C@@H]2CCC[C@H]2OC(F)F)cc1C(N)=O] | 32 | (abs) | c | bb | ccc | A | B* |
| 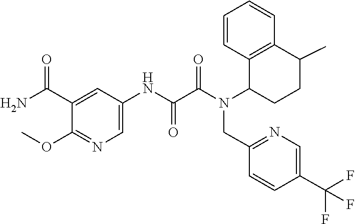 [SMILES: COc1ncc(NC(=O)C(=O)N(Cc2ccc(C(F)(F)F)cn2)C2CCC(C)c3ccccc32)cc1C(N)=O] | 33 | | c | bb | ccc | C | A* |
| 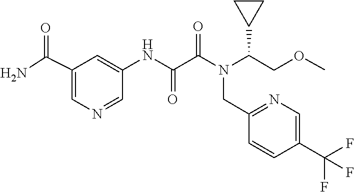 [SMILES: COC[C@@H](C1CC1)N(Cc1ccc(C(F)(F)F)cn1)C(=O)C(=O)Nc1cncc(C(N)=O)c1] | 34 | (abs) | c | bb | bbb | B | B* |
| 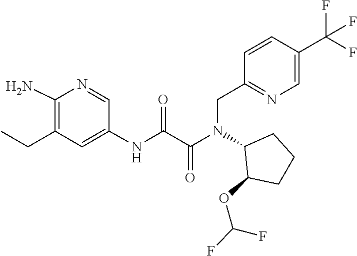 [SMILES: CCc1cc(NC(=O)C(=O)N(Cc2ccc(C(F)(F)F)cn2)[C@@H]2CCC[C@H]2OC(F)F)cnc1N] | 35 | (abs) | c | bb | ccc | A | B* |
| 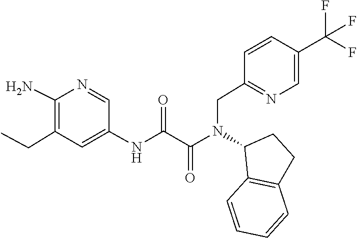 [SMILES: CCc1cc(NC(=O)C(=O)N(Cc2ccc(C(F)(F)F)cn2)[C@@H]2CCc3ccccc32)cnc1N] | 36 | (abs) | c | bb | bbb | A | A* |
| 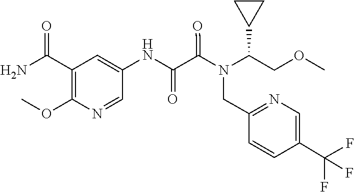 [SMILES: COC[C@@H](C1CC1)N(Cc1ccc(C(F)(F)F)cn1)C(=O)C(=O)Nc1cnc(OC)c(C(N)=O)c1] | 37 | (abs) | c | bb | aaa | B | A* |
| 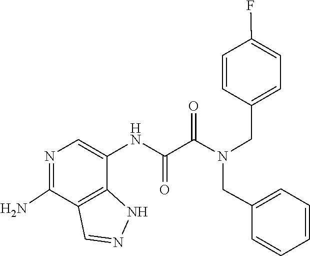 [SMILES: Nc1ncc(NC(=O)C(=O)N(Cc2ccccc2)Cc2ccc(F)cc2)c2[nH]ncc12] | 38 | | c | aa | aaa | A | A* |
| 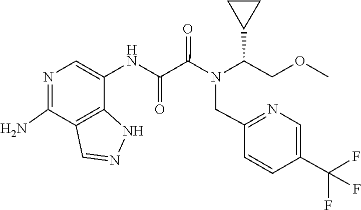 [SMILES: COC[C@@H](C1CC1)N(Cc1ccc(C(F)(F)F)cn1)C(=O)C(=O)Nc1cnc(N)c2cn[nH]c12] | 39 | (abs) | b | aa | aaa | B | A* |
| 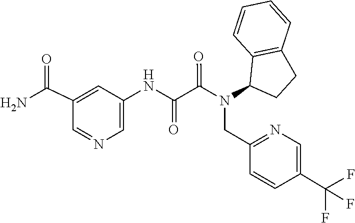 [SMILES: NC(=O)c1cncc(NC(=O)C(=O)N(Cc2ccc(C(F)(F)F)cn2)[C@@H]2CCc3ccccc32)c1] | 40 | (abs) | c | bb | ccc | B | A* |
| 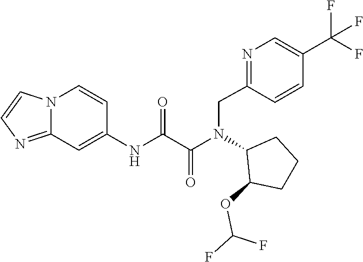 [SMILES: O=C(Nc1ccn2ccnc2c1)C(=O)N(Cc1ccc(C(F)(F)F)cn1)[C@@H]1CCC[C@H]1OC(F)F] | 41 | (and) | c | bb | ccc | A | B* |
| 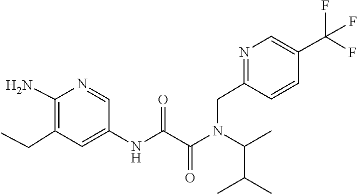 [SMILES: CCc1cc(NC(=O)C(=O)N(Cc2ccc(C(F)(F)F)cn2)C(C)C(C)C)cnc1N] | 42 | | c | bb | ccc | B | B* |
| 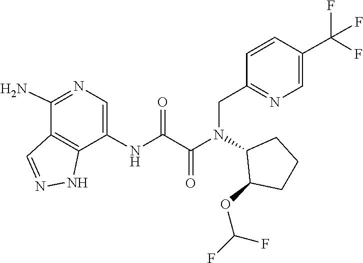 [SMILES: Nc1ncc(NC(=O)C(=O)N(Cc2ccc(C(F)(F)F)cn2)[C@@H]2CCC[C@H]2OC(F)F)c2[nH]ncc12] | 43 | (abs) | b | aa | aaa | C | A* |
| 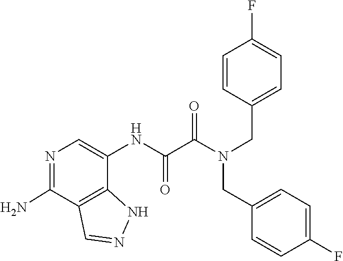 [SMILES: Nc1ncc(NC(=O)C(=O)N(Cc2ccc(F)cc2)Cc2ccc(F)cc2)c2[nH]ncc12] | 44 | | c | aa | aaa | A | |
| 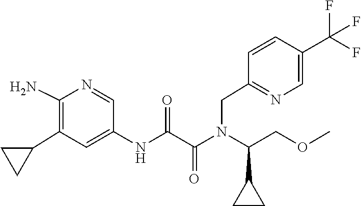 [SMILES: COC[C@@H](C1CC1)N(Cc1ccc(C(F)(F)F)cn1)C(=O)C(=O)Nc1cnc(N)c(C2CC2)c1] | 45 | (abs) | c | bb | ccc | A | B* |
| 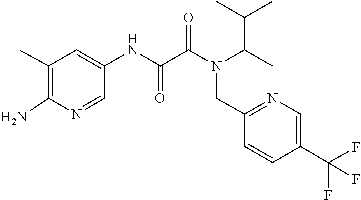 [SMILES: Cc1cc(NC(=O)C(=O)N(Cc2ccc(C(F)(F)F)cn2)C(C)C(C)C)cnc1N] | 46 | | b | bb | ccc | C | A* |
| 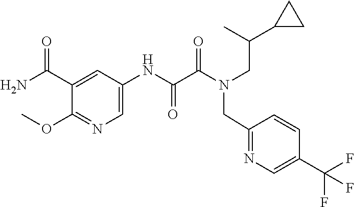 [SMILES: COc1ncc(NC(=O)C(=O)N(Cc2ccc(C(F)(F)F)cn2)CC(C)C2CC2)cc1C(N)=O] | 47 | (abs) | c | aa | ccc | A | B* |
| 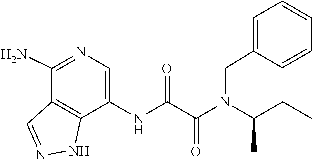 [SMILES: CC[C@@H](C)N(Cc1ccccc1)C(=O)C(=O)Nc1cnc(N)c2cn[nH]c12] | 48 | (abs) | c | aa | ccc | A | B* |
| 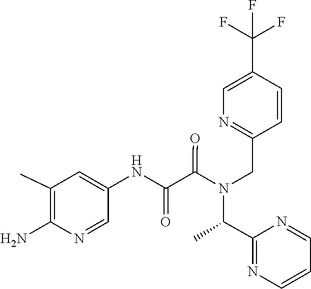 [SMILES: Cc1cc(NC(=O)C(=O)N(Cc2ccc(C(F)(F)F)cn2)[C@@H](C)c2ncccn2)cnc1N] | 49 | (abs) | b | bb | ccc | C | B* |
| 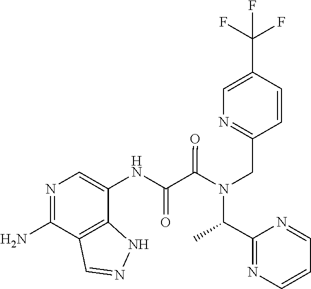 [SMILES: C[C@@H](c1ncccn1)N(Cc1ccc(C(F)(F)F)cn1)C(=O)C(=O)Nc1cnc(N)c2cn[nH]c12] | 50 | (abs) | b | aa | aaa | C | A* |
| 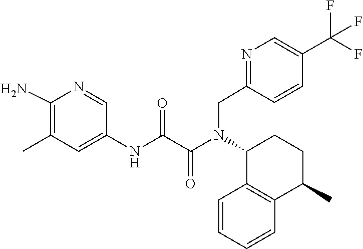 [SMILES: Cc1cc(NC(=O)C(=O)N(Cc2ccc(C(F)(F)F)cn2)[C@@H]2CC[C@@H](C)c3ccccc32)cnc1N] | 51 | (abs) | b | aa | aaa | C | A* |
| 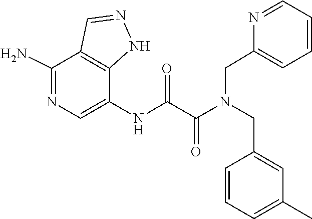 [SMILES: Cc1cccc(CN(Cc2ccccn2)C(=O)C(=O)Nc2cnc(N)c3cn[nH]c23)c1] | 52 | | b | aa | aaa | B | A* |
| 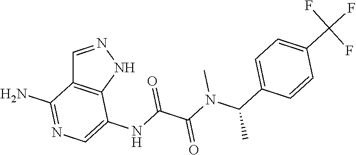 [SMILES: C[C@@H](c1ccc(C(F)(F)F)cc1)N(C)C(=O)C(=O)Nc1cnc(N)c2cn[nH]c12] | 53 | (abs) | c | bb | ccc | A | B* |
| 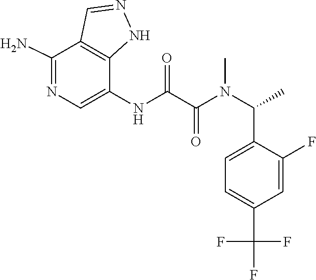 [SMILES: C[C@H](c1ccc(C(F)(F)F)cc1F)N(C)C(=O)C(=O)Nc1cnc(N)c2cn[nH]c12] | 54 | (abs) | b | aa | aaa | A | A* |
| 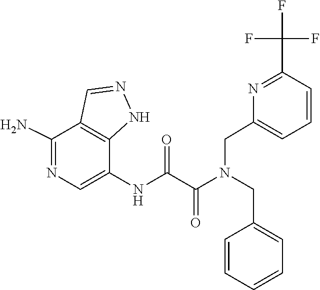 [SMILES: Nc1ncc(NC(=O)C(=O)N(Cc2ccccc2)Cc2cccc(C(F)(F)F)n2)c2[nH]ncc12] | 55 | | b | aa | aaa | B | A* |
| 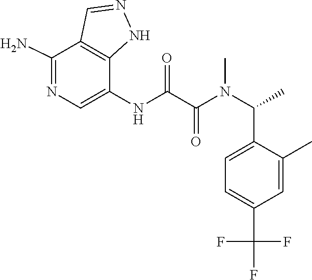 [SMILES: Cc1cc(C(F)(F)F)ccc1[C@@H](C)N(C)C(=O)C(=O)Nc1cnc(N)c2cn[nH]c12] | 56 | (abs) | b | aa | aaa | B | A* |
| 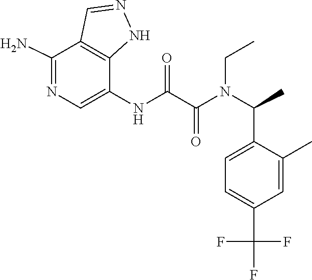 [SMILES: CCN(C(=O)C(=O)Nc1cnc(N)c2cn[nH]c12)[C@@H](C)c1ccc(C(F)(F)F)cc1C] | 57 | (abs) | c | aa | aaa | B | A* |
| 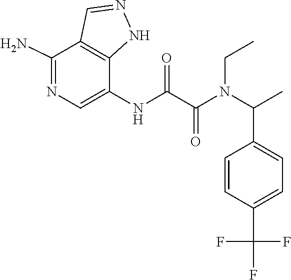 [SMILES: CCN(C(=O)C(=O)Nc1cnc(N)c2cn[nH]c12)C(C)c1ccc(C(F)(F)F)cc1] | 58 | (and) | c | aa | aaa | B | A* |
| 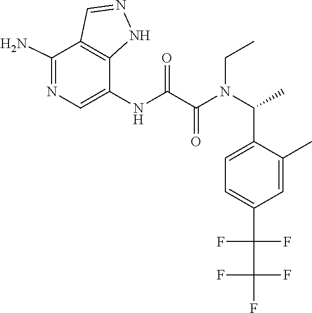 [SMILES: CCN(C(=O)C(=O)Nc1cnc(N)c2cn[nH]c12)[C@H](C)c1ccc(C(F)(F)C(F)(F)F)cc1C] | 59 | (abs) | b | aa | aaa | C | A* |
| 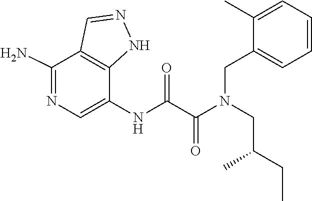 [SMILES: CC[C@H](C)CN(Cc1ccccc1C)C(=O)C(=O)Nc1cnc(N)c2cn[nH]c12] | 60 | (abs) | b | aa | aaa | A | A* |
| 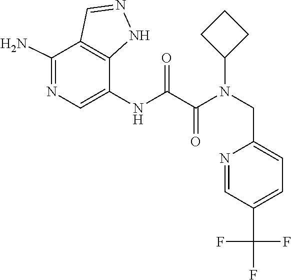 [SMILES: Nc1ncc(NC(=O)C(=O)N(Cc2ccc(C(F)(F)F)cn2)C2CCC2)c2[nH]ncc12] | 61 | | b | aa | aaa | B | A* |
| 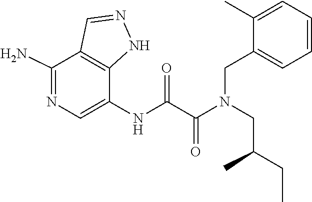 [SMILES: CC[C@@H](C)CN(Cc1ccccc1C)C(=O)C(=O)Nc1cnc(N)c2cn[nH]c12] | 62 | (abs) | b | aa | aaa | A | A* |
| 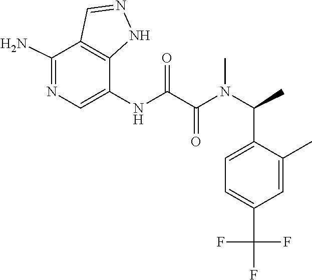 [SMILES: Cc1cc(C(F)(F)F)ccc1[C@H](C)N(C)C(=O)C(=O)Nc1cnc(N)c2cn[nH]c12] | 63 | (abs) | c | aa | aaa | A | A* |
| 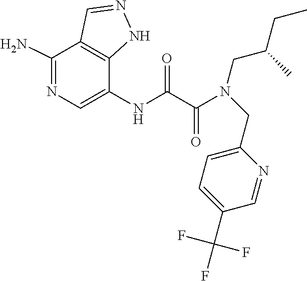 [SMILES: CC[C@H](C)CN(Cc1ccc(C(F)(F)F)cn1)C(=O)C(=O)Nc1cnc(N)c2cn[nH]c12] | 64 | (abs) | a | aa | aaa | C | A* |
| 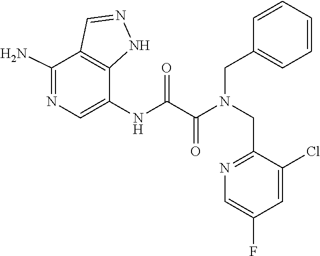 [SMILES: Nc1ncc(NC(=O)C(=O)N(Cc2ccccc2)Cc2ncc(F)cc2Cl)c2[nH]ncc12] | 65 | | b | aa | aaa | C | A* |
| 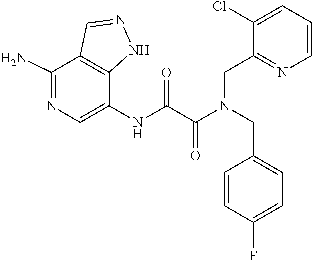 [SMILES: Nc1ncc(NC(=O)C(=O)N(Cc2ccc(F)cc2)Cc2ncccc2Cl)c2[nH]ncc12] | 66 | | b | aa | aaa | B | A* |
| 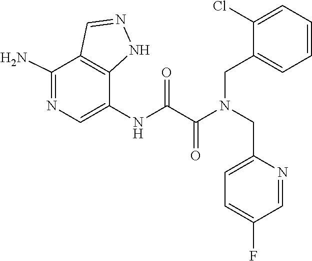 [SMILES: Nc1ncc(NC(=O)C(=O)N(Cc2ccc(F)cn2)Cc2ccccc2Cl)c2[nH]ncc12] | 67 | | C | aa | aaa | B | A* |
| 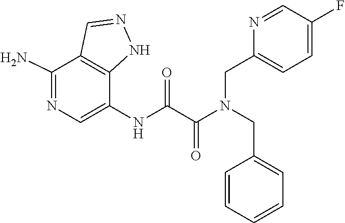 [SMILES: Nc1ncc(NC(=O)C(=O)N(Cc2ccccc2)Cc2ccc(F)cn2)c2[nH]ncc12] | 68 | | c | aa | aaa | A | A* |
| 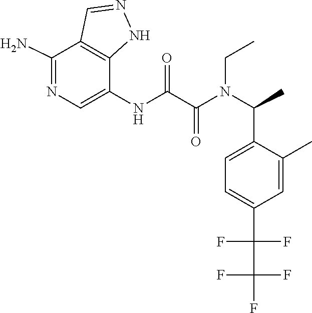 [SMILES: CCN(C(=O)C(=O)Nc1cnc(N)c2cn[nH]c12)[C@@H](C)c1ccc(C(F)(F)C(F)(F)F)cc1C] | 69 | (abs) | c | bb | aaa | B | A* |
| 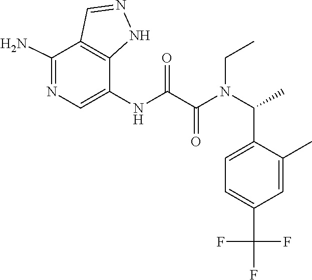 [SMILES: CCN(C(=O)C(=O)Nc1cnc(N)c2cn[nH]c12)[C@H](C)c1ccc(C(F)(F)F)cc1C] | 70 | (abs) | b | aa | aaa | C | A* |
| 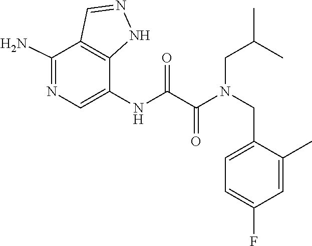 [SMILES: Cc1cc(F)ccc1CN(CC(C)C)C(=O)C(=O)Nc1cnc(N)c2cn[nH]c12] | 71 | | C | aa | aaa | A | A* |
| 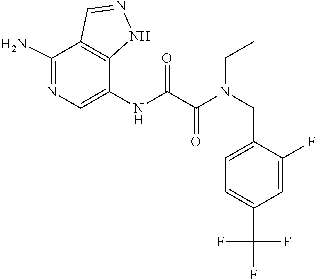 [SMILES: CCN(Cc1ccc(C(F)(F)F)cc1F)C(=O)C(=O)Nc1cnc(N)c2cn[nH]c12] | 72 | | b | aa | aaa | A | A* |
| 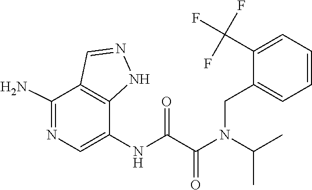 [SMILES: CC(C)N(Cc1ccccc1C(F)(F)F)C(=O)C(=O)Nc1cnc(N)c2cn[nH]c12] | 73 | | b | aa | aaa | B | A* |
| 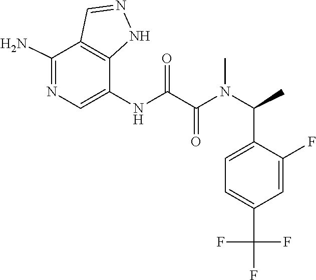 [SMILES: C[C@@H](c1ccc(C(F)(F)F)cc1F)N(C)C(=O)C(=O)Nc1cnc(N)c2cn[nH]c12] | 74 | (abs) | c | bb | ccc | A | B* |
| 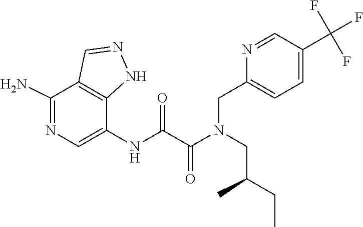 [SMILES: CC[C@@H](C)CN(Cc1ccc(C(F)(F)F)cn1)C(=O)C(=O)Nc1cnc(N)c2cn[nH]c12] | 75 | (abs) | b | aa | aaa | C | A* |
| 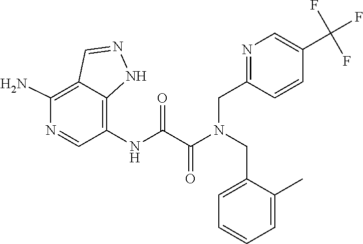 [SMILES: Cc1ccccc1CN(Cc1ccc(C(F)(F)F)cn1)C(=O)C(=O)Nc1cnc(N)c2cn[nH]c12] | 76 | | b | aa | aaa | C | A* |
| 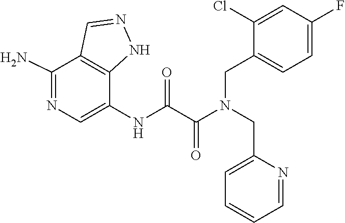 [SMILES: Nc1ncc(NC(=O)C(=O)N(Cc2ccccn2)Cc2ccc(F)cc2Cl)c2[nH]ncc12] | 77 | | b | aa | aaa | B | A* |
| 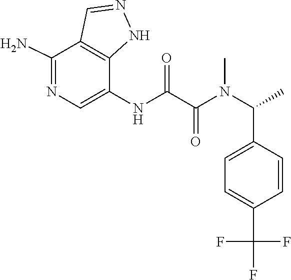 [SMILES: C[C@H](c1ccc(C(F)(F)F)cc1)N(C)C(=O)C(=O)Nc1cnc(N)c2cn[nH]c12] | 78 | (abs) | c | aa | aaa | A | A* |
| 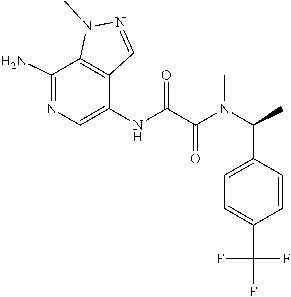 [SMILES: C[C@@H](c1ccc(C(F)(F)F)cc1)N(C)C(=O)C(=O)Nc1cnc(N)c2c1cnn2C] | 79 | (abs) | c | bb | ccc | A | B* |
| 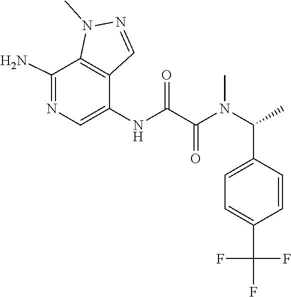 [SMILES: C[C@H](c1ccc(C(F)(F)F)cc1)N(C)C(=O)C(=O)Nc1cnc(N)c2c1cnn2C] | 80 | (abs) | c | aa | aaa | A | A* |
| 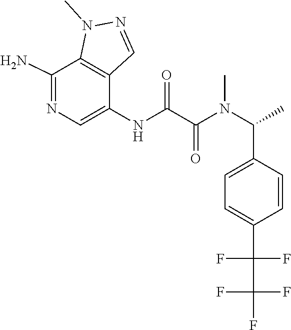 [SMILES: C[C@H](c1ccc(C(F)(F)C(F)(F)F)cc1)N(C)C(=O)C(=O)Nc1cnc(N)c2c1cnn2C] | 81 | (abs) | b | aa | aaa | A | A* |
| 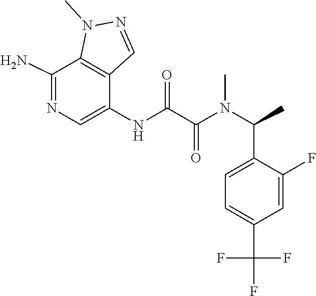 [SMILES: C[C@@H](c1ccc(C(F)(F)F)cc1F)N(C)C(=O)C(=O)Nc1cnc(N)c2c1cnn2C] | 82 | (abs) | c | bb | ccc | B | B* |
| 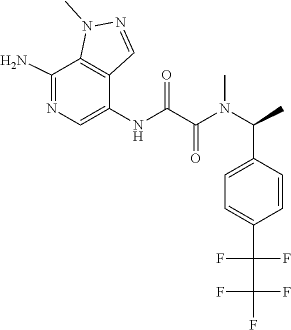 [SMILES: C[C@@H](c1ccc(C(F)(F)C(F)(F)F)cc1)N(C)C(=O)C(=O)Nc1cnc(N)c2c1cnn2C] | 83 | (abs) | c | bb | ccc | A | B* |
| 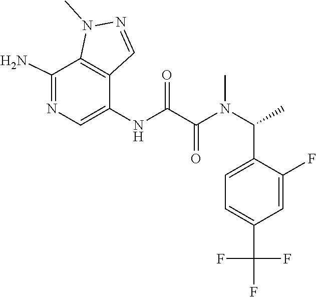 [SMILES: C[C@H](c1ccc(C(F)(F)F)cc1F)N(C)C(=O)C(=O)Nc1cnc(N)c2c1cnn2C] | 84 | (abs) | b | aa | aaa | A | A* |
| 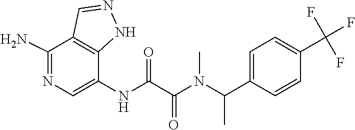 [SMILES: CC(c1ccc(C(F)(F)F)cc1)N(C)C(=O)C(=O)Nc1cnc(N)c2cn[nH]c12] | 85 | | c | aa | aaa | A | A* |
| 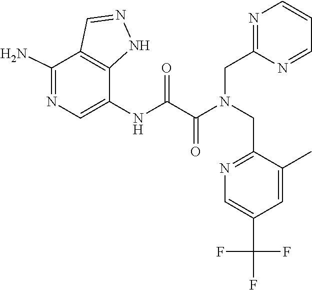 [SMILES: Cc1cc(C(F)(F)F)cnc1CN(Cc1ncccn1)C(=O)C(=O)Nc1cnc(N)c2cn[nH]c12] | 86 | | b | aa | aaa | C | A* |
| 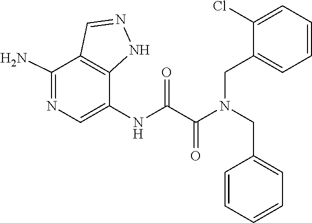 [SMILES: Nc1ncc(NC(=O)C(=O)N(Cc2ccccc2)Cc2ccccc2Cl)c2[nH]ncc12] | 87 | | b | aa | aaa | B | A* |
| 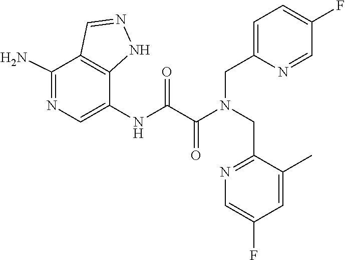 [SMILES: Cc1cc(F)cnc1CN(Cc1ccc(F)cn1)C(=O)C(=O)Nc1cnc(N)c2cn[nH]c12] | 88 | | b | aa | aaa | B | A* |
| 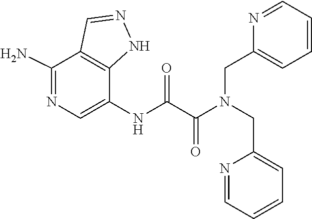 [SMILES: Nc1ncc(NC(=O)C(=O)N(Cc2ccccn2)Cc2ccccn2)c2[nH]ncc12] | 89 | | c | aa | aaa | A | A* |
| 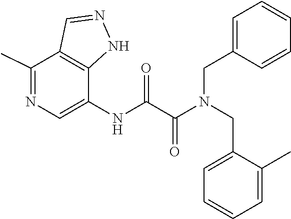 | 90 | | c | aa | ccc | A | B* |
| 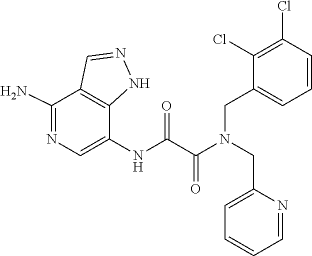 [SMILES: Nc1ncc(NC(=O)C(=O)N(Cc2ccccn2)Cc2cccc(Cl)c2Cl)c2[nH]ncc12] | 91 | | a | aa | aaa | C | A* |
| 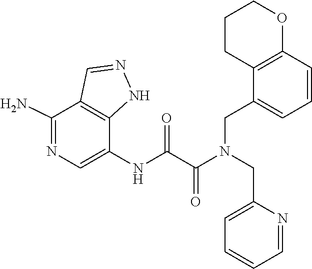 [SMILES: Nc1ncc(NC(=O)C(=O)N(Cc2ccccn2)Cc2cccc3c2CCCO3)c2[nH]ncc12] | 92 | | a | aa | aaa | C | A* |
| 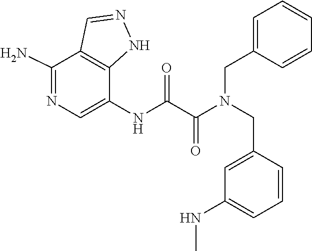 [SMILES: CNc1cccc(CN(Cc2ccccc2)C(=O)C(=O)Nc2cnc(N)c3cn[nH]c23)c1] | 93 | | b | aa | aaa | B | A* |
| 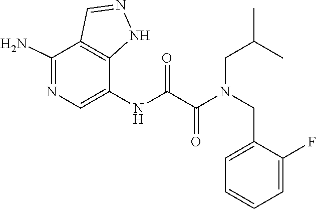 [SMILES: CC(C)CN(Cc1ccccc1F)C(=O)C(=O)Nc1cnc(N)c2cn[nH]c12] | 94 | | b | aa | aaa | B | A* |
| 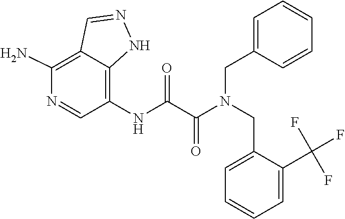 [SMILES: Nc1ncc(NC(=O)C(=O)N(Cc2ccccc2)Cc2ccccc2C(F)(F)F)c2[nH]ncc12] | 95 | | a | aa | aaa | C | A* |
| 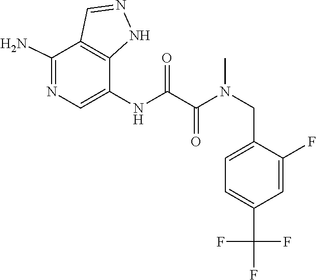 [SMILES: CN(Cc1ccc(C(F)(F)F)cc1F)C(=O)C(=O)Nc1cnc(N)c2cn[nH]c12] | 96 | | c | bb | ccc | A | B* |
| 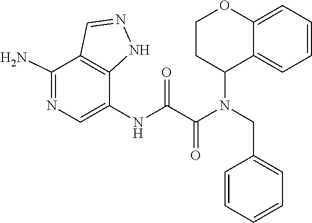 [SMILES: Nc1ncc(NC(=O)C(=O)N(Cc2ccccc2)C2CCOc3ccccc32)c2[nH]ncc12] | 97 | | b | aa | aaa | B | A* |
| 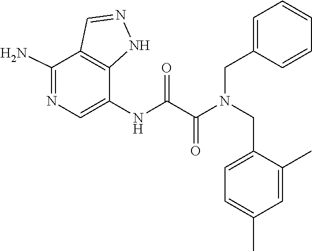 [SMILES: Cc1ccc(CN(Cc2ccccc2)C(=O)C(=O)Nc2cnc(N)c3cn[nH]c23)c(C)c1] | 98 | | b | aa | aaa | B | A* |
| 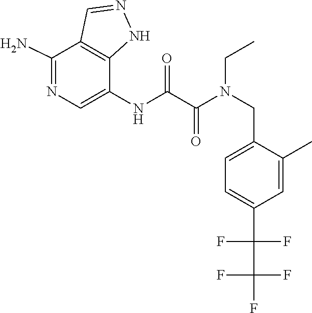 [SMILES: CCN(Cc1ccc(C(F)(F)C(F)(F)F)cc1C)C(=O)C(=O)Nc1cnc(N)c2cn[nH]c12] | 99 | | c | aa | aaa | A | A* |
| 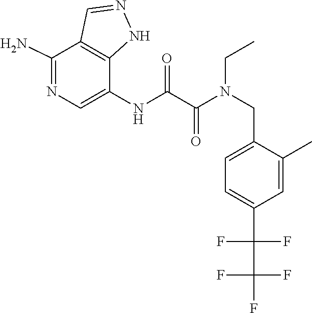 [SMILES: CCN(Cc1ccc(C(F)(F)C(F)(F)F)cc1C)C(=O)C(=O)Nc1cnc(N)c2cn[nH]c12] | 100 | | c | aa | aaa | A | A* |
| 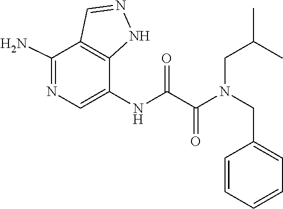 [SMILES: CC(C)CN(Cc1ccccc1)C(=O)C(=O)Nc1cnc(N)c2cn[nH]c12] | 101 | | c | aa | bbb | A | A* |
| 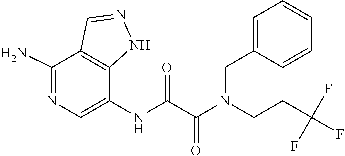 [SMILES: Nc1ncc(NC(=O)C(=O)N(CCC(F)(F)F)Cc2ccccc2)c2[nH]ncc12] | 102 | | c | bb | ccc | A | B* |
| 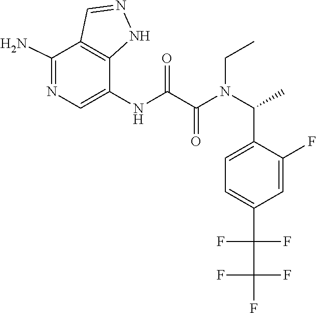 [SMILES: CCN(C(=O)C(=O)Nc1cnc(N)c2cn[nH]c12)[C@H](C)c1ccc(C(F)(F)C(F)(F)F)cc1F] | 103 | (abs) | b | aa | aaa | C | A* |
| 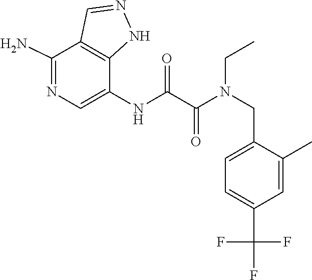 [SMILES: CCN(Cc1ccc(C(F)(F)F)cc1C)C(=O)C(=O)Nc1cnc(N)c2cn[nH]c12] | 104 | | c | aa | aaa | A | A* |
| 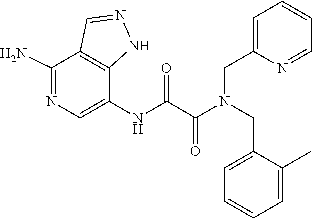 [SMILES: Cc1ccccc1CN(Cc1ccccn1)C(=O)C(=O)Nc1cnc(N)c2cn[nH]c12] | 105 | | | | | | |
| 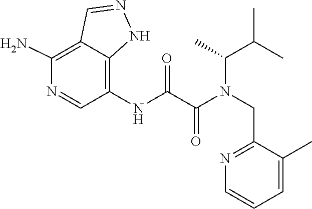 [SMILES: Cc1cccnc1CN(C(=O)C(=O)Nc1cnc(N)c2cn[nH]c12)[C@H](C)C(C)C] | 106 | (or) | b | aa | aaa | C | A* |
| 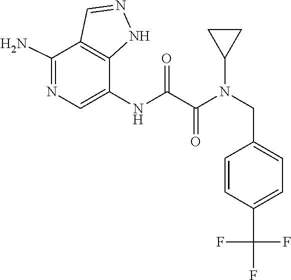 [SMILES: Nc1ncc(NC(=O)C(=O)N(Cc2ccc(C(F)(F)F)cc2)C2CC2)c2[nH]ncc12] | 107 | | b | aa | aaa | C | A* |
| 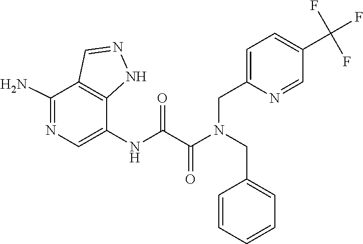 [SMILES: Nc1ncc(NC(=O)C(=O)N(Cc2ccccc2)Cc2ccc(C(F)(F)F)cn2)c2[nH]ncc12] | 108 | | b | aa | aaa | B | A* |
| 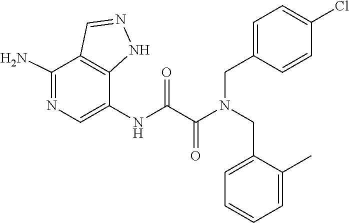 [SMILES: Cc1ccccc1CN(Cc1ccc(Cl)cc1)C(=O)C(=O)Nc1cnc(N)c2cn[nH]c12] | 109 | | b | aa | aaa | C | A* |
| 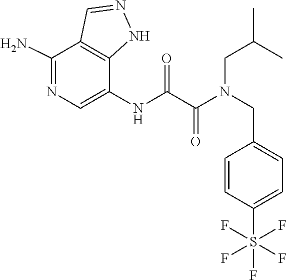 [SMILES: CC(C)CN(Cc1ccc(S(F)(F)(F)(F)F)cc1)C(=O)C(=O)Nc1cnc(N)c2cn[nH]c12] | 110 | | b | aa | aaa | C | A* |
| 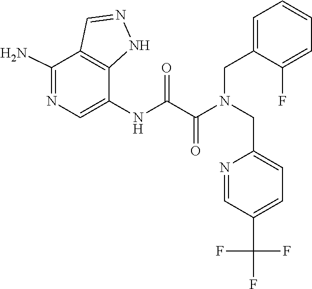 [SMILES: Nc1ncc(NC(=O)C(=O)N(Cc2ccc(C(F)(F)F)cn2)Cc2ccccc2F)c2[nH]ncc12] | 111 | | a | aa | aaa | C | A* |
| 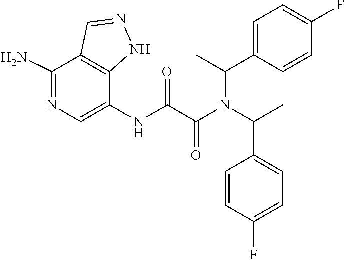 [SMILES: CC(c1ccc(F)cc1)N(C(=O)C(=O)Nc1cnc(N)c2cn[nH]c12)C(C)c1ccc(F)cc1] | 112 | | c | aa | aaa | B | A* |
| 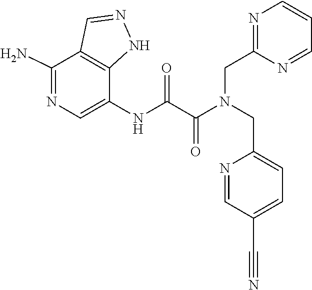 [SMILES: N#Cc1ccc(CN(Cc2ncccn2)C(=O)C(=O)Nc2cnc(N)c3cn[nH]c23)nc1] | 113 | | c | bb | ccc | B | B* |
| 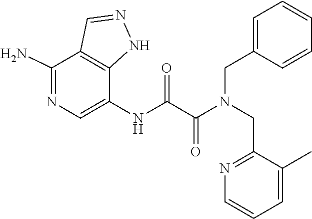 [SMILES: Cc1cccnc1CN(Cc1ccccc1)C(=O)C(=O)Nc1cnc(N)c2cn[nH]c12] | 114 | | b | aa | aaa | C | A* |
| 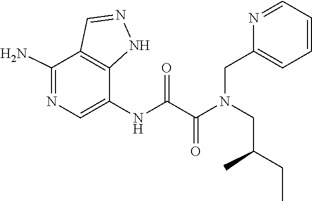 [SMILES: CC[C@@H](C)CN(Cc1ccccn1)C(=O)C(=O)Nc1cnc(N)c2cn[nH]c12] | 115 | (abs) | c | aa | aaa | A | A* |
| 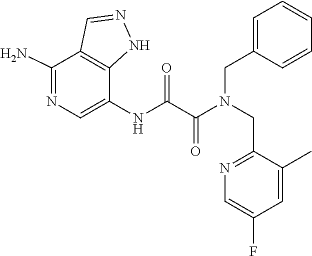 [SMILES: Cc1cc(F)cnc1CN(Cc1ccccc1)C(=O)C(=O)Nc1cnc(N)c2cn[nH]c12] | 116 | | b | aa | aaa | B | A* |
| 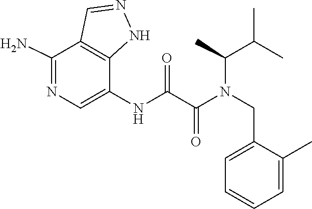 [SMILES: Cc1ccccc1CN(C(=O)C(=O)Nc1cnc(N)c2cn[nH]c12)[C@@H](C)C(C)C] | 117 | (abs) | c | aa | bbb | B | B* |
| 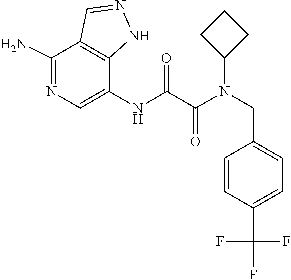 [SMILES: Nc1ncc(NC(=O)C(=O)N(Cc2ccc(C(F)(F)F)cc2)C2CCC2)c2[nH]ncc12] | 118 | | c | aa | bbb | B | A* |
| 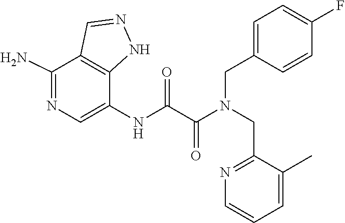 [SMILES: Cc1cccnc1CN(Cc1ccc(F)cc1)C(=O)C(=O)Nc1cnc(N)c2cn[nH]c12] | 119 | | b | aa | aaa | A | A* |
| 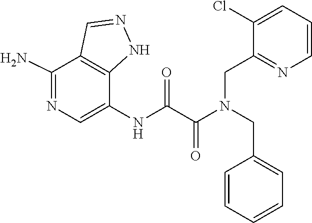 [SMILES: Nc1ncc(NC(=O)C(=O)N(Cc2ccccc2)Cc2ncccc2Cl)c2[nH]ncc12] | 120 | | b | aa | aaa | B | A* |
| 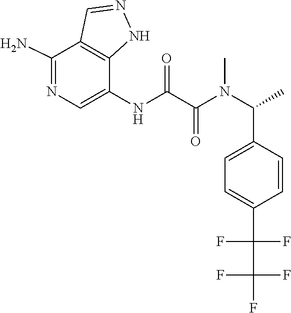 [SMILES: C[C@H](c1ccc(C(F)(F)C(F)(F)F)cc1)N(C)C(=O)C(=O)Nc1cnc(N)c2cn[nH]c12] | 121 | (abs) | b | aa | aaa | B | A* |
| 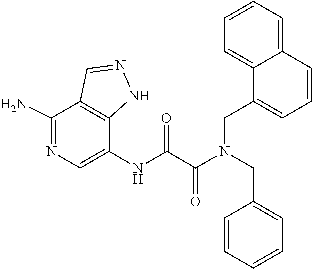 [SMILES: Nc1ncc(NC(=O)C(=O)N(Cc2ccccc2)Cc2cccc3ccccc23)c2[nH]ncc12] | 122 | | b | aa | aaa | B | A* |
| 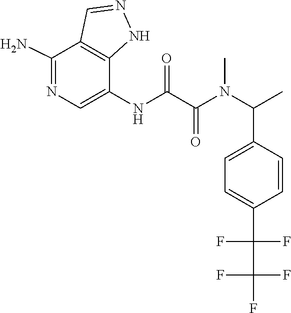 [SMILES: CC(c1ccc(C(F)(F)C(F)(F)F)cc1)N(C)C(=O)C(=O)Nc1cnc(N)c2cn[nH]c12] | 123 | | b | aa | aaa | B | A* |
| 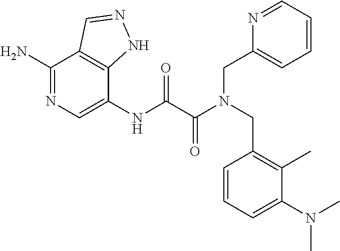 [SMILES: Cc1c(CN(Cc2ccccn2)C(=O)C(=O)Nc2cnc(N)c3cn[nH]c23)cccc1N(C)C] | 124 | | b | aa | aaa | C | A* |
| 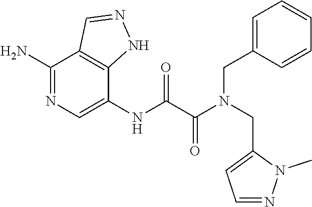 [SMILES: Cn1nccc1CN(Cc1ccccc1)C(=O)C(=O)Nc1cnc(N)c2cn[nH]c12] | 125 | | b | aa | aaa | B | A* |
| 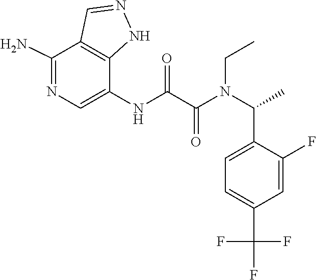 [SMILES: CCN(C(=O)C(=O)Nc1cnc(N)c2cn[nH]c12)[C@H](C)c1ccc(C(F)(F)F)cc1F] | 126 | (abs) | b | aa | aaa | B | A* |
| 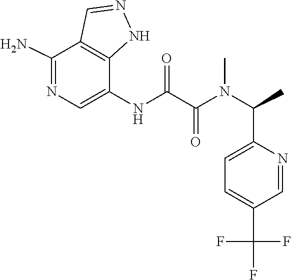 [SMILES: C[C@@H](c1ccc(C(F)(F)F)cn1)N(C)C(=O)C(=O)Nc1cnc(N)c2cn[nH]c12] | 127 | (abs) | c | bb | ccc | A | B* |
| 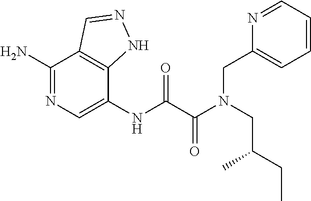 [SMILES: CC[C@H](C)CN(Cc1ccccn1)C(=O)C(=O)Nc1cnc(N)c2cn[nH]c12] | 128 | (abs) | c | aa | aaa | A | A* |
| 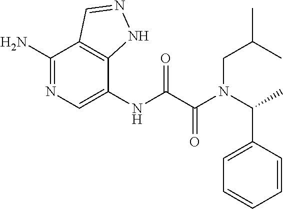 [SMILES: CC(C)CN(C(=O)C(=O)Nc1cnc(N)c2cn[nH]c12)[C@H](C)c1ccccc1] | 129 | (abs) | c | aa | aaa | A | A* |
| 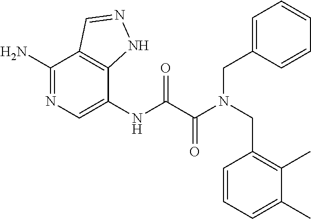 [SMILES: Cc1cccc(CN(Cc2ccccc2)C(=O)C(=O)Nc2cnc(N)c3cn[nH]c23)c1C] | 130 | | b | aa | aaa | B | A* |
| 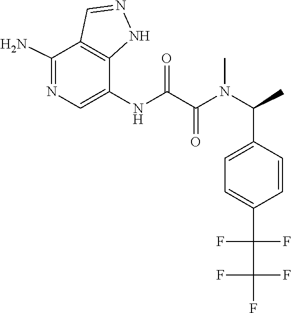 [SMILES: C[C@@H](c1ccc(C(F)(F)C(F)(F)F)cc1)N(C)C(=O)C(=O)Nc1cnc(N)c2cn[nH]c12] | 131 | (abs) | c | bb | ccc | A | A* |
| 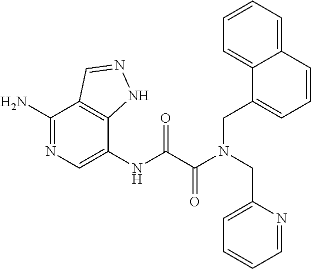 [SMILES: Nc1ncc(NC(=O)C(=O)N(Cc2ccccn2)Cc2cccc3ccccc23)c2[nH]ncc12] | 132 | | b | aa | aaa | A | A* |
| 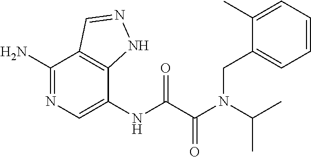 [SMILES: Cc1ccccc1CN(C(=O)C(=O)Nc1cnc(N)c2cn[nH]c12)C(C)C] | 133 | | c | aa | aaa | A | A* |
| 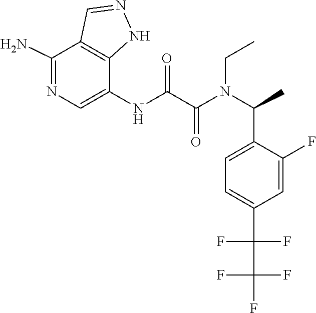 [SMILES: CCN(C(=O)C(=O)Nc1cnc(N)c2cn[nH]c12)[C@@H](C)c1ccc(C(F)(F)C(F)(F)F)cc1F] | 134 | (abs) | c | bb | ccc | A | B* |
| 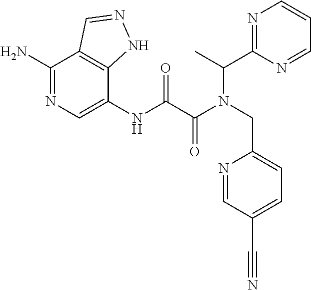 [SMILES: CC(c1ncccn1)N(Cc1ccc(C#N)cn1)C(=O)C(=O)Nc1cnc(N)c2cn[nH]c12] | 135 | | c | bb | ccc | C | B* |
| 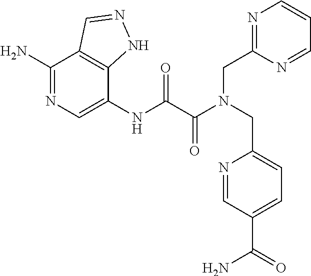 [SMILES: NC(=O)c1ccc(CN(Cc2ncccn2)C(=O)C(=O)Nc2cnc(N)c3cn[nH]c23)nc1] | 136 | | c | cc | ccc | C | C* |
| 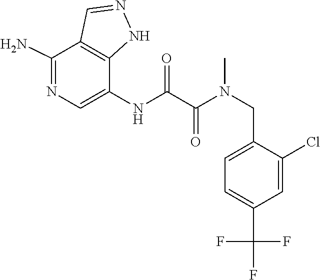 [SMILES: CN(Cc1ccc(C(F)(F)F)cc1Cl)C(=O)C(=O)Nc1cnc(N)c2cn[nH]c12] | 137 | | c | aa | bbb | A | B* |
| 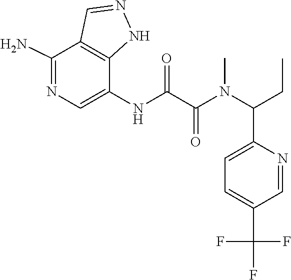 [SMILES: CCC(c1ccc(C(F)(F)F)cn1)N(C)C(=O)C(=O)Nc1cnc(N)c2cn[nH]c12] | 138 | | c | bb | bbb | A | B* |
| 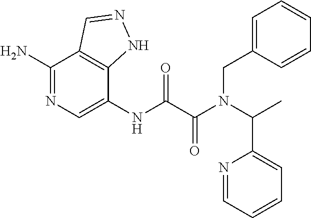 [SMILES: CC(c1ccccn1)N(Cc1ccccc1)C(=O)C(=O)Nc1cnc(N)c2cn[nH]c12] | 139 | | b | aa | aaa | A | A* |
| 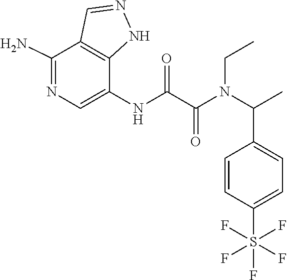 [SMILES: CCN(C(=O)C(=O)Nc1cnc(N)c2cn[nH]c12)C(C)c1ccc(S(F)(F)(F)(F)F)cc1] | 140 | | b | aa | aaa | C | A* |
| 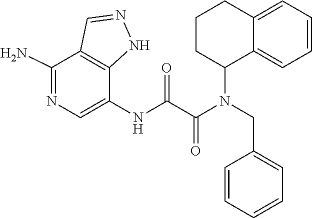 [SMILES: Nc1ncc(NC(=O)C(=O)N(Cc2ccccc2)C2CCCc3ccccc32)c2[nH]ncc12] | 141 | | b | aa | aaa | C | A* |
| 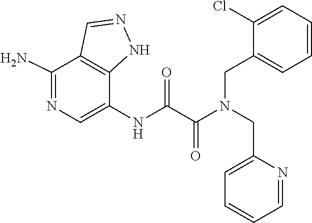 [SMILES: Nc1ncc(NC(=O)C(=O)N(Cc2ccccn2)Cc2ccccc2Cl)c2[nH]ncc12] | 142 | | b | aa | aaa | B | A* |
| 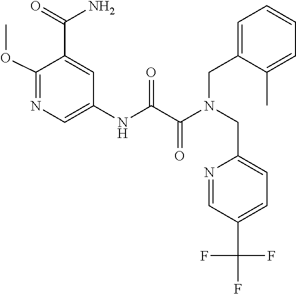 [SMILES: COc1ncc(NC(=O)C(=O)N(Cc2ccc(C(F)(F)F)cn2)Cc2ccccc2C)cc1C(N)=O] | 143 | | c | aa | aaa | A | A* |
| 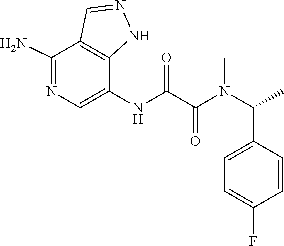 [SMILES: C[C@H](c1ccc(F)cc1)N(C)C(=O)C(=O)Nc1cnc(N)c2cn[nH]c12] | 144 | (abs) | c | aa | ccc | A | A* |
| 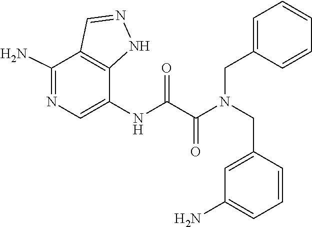 [SMILES: Nc1cccc(CN(Cc2ccccc2)C(=O)C(=O)Nc2cnc(N)c3cn[nH]c23)c1] | 145 | | c | aa | aaa | B | A* |
| 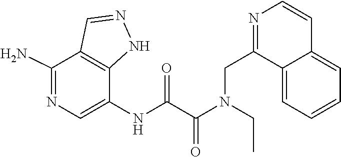 [SMILES: CCN(Cc1nccc2ccccc12)C(=O)C(=O)Nc1cnc(N)c2cn[nH]c12] | 146 | | c | aa | bbb | A | B* |
| 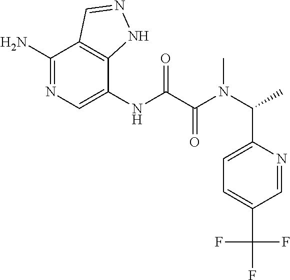 [SMILES: C[C@H](c1ccc(C(F)(F)F)cn1)N(C)C(=O)C(=O)Nc1cnc(N)c2cn[nH]c12] | 147 | (abs) | c | aa | aaa | A | A* |
| 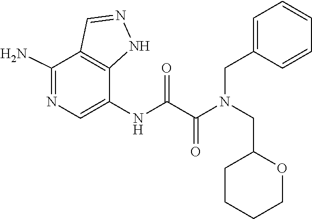 [SMILES: Nc1ncc(NC(=O)C(=O)N(Cc2ccccc2)CC2CCCCO2)c2[nH]ncc12] | 148 | | c | aa | bbb | A | B* |
| 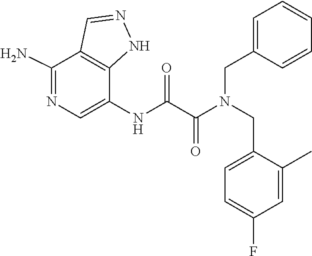 [SMILES: Cc1cc(F)ccc1CN(Cc1ccccc1)C(=O)C(=O)Nc1cnc(N)c2cn[nH]c12] | 149 | | b | aa | aaa | B | A* |
| 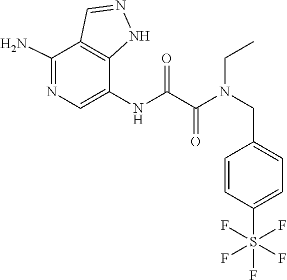 [SMILES: CCN(Cc1ccc(S(F)(F)(F)(F)F)cc1)C(=O)C(=O)Nc1cnc(N)c2cn[nH]c12] | 150 | | b | aa | aaa | C | A* |
| 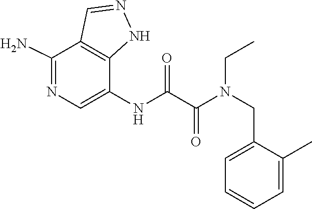 [SMILES: CCN(Cc1ccccc1C)C(=O)C(=O)Nc1cnc(N)c2cn[nH]c12] | 151 | | c | aa | bbb | A | A* |
| 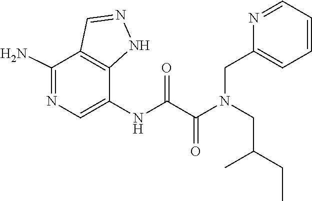 [SMILES: CCC(C)CN(Cc1ccccn1)C(=O)C(=O)Nc1cnc(N)c2cn[nH]c12] | 152 | | c | aa | aaa | A | A* |
| 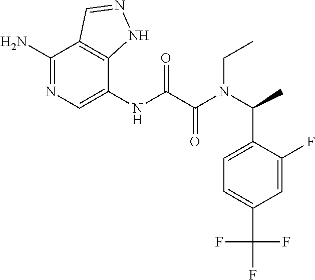 [SMILES: CCN(C(=O)C(=O)Nc1cnc(N)c2cn[nH]c12)[C@@H](C)c1ccc(C(F)(F)F)cc1F] | 153 | (abs) | c | bb | ccc | A | B* |
| 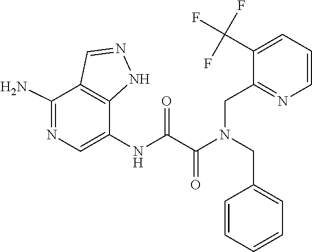 [SMILES: Nc1ncc(NC(=O)C(=O)N(Cc2ccccc2)Cc2ncccc2C(F)(F)F)c2[nH]ncc12] | 154 | | b | aa | aaa | A | A* |
| 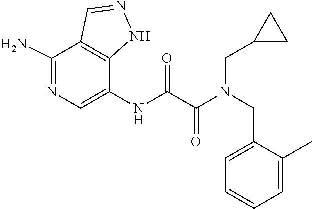 [SMILES: Cc1ccccc1CN(CC1CC1)C(=O)C(=O)Nc1cnc(N)c2cn[nH]c12] | 155 | | c | aa | aaa | A | A* |
| 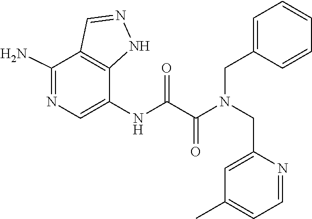 [SMILES: Cc1ccnc(CN(Cc2ccccc2)C(=O)C(=O)Nc2cnc(N)c3cn[nH]c23)c1] | 156 | | c | aa | aaa | A | A* |
| 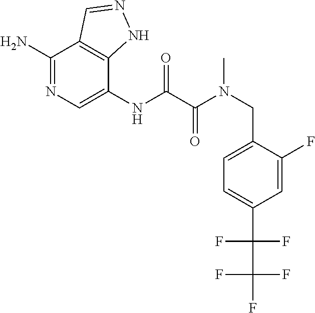 [SMILES: CN(Cc1ccc(C(F)(F)C(F)(F)F)cc1F)C(=O)C(=O)Nc1cnc(N)c2cn[nH]c12] | 157 | | c | aa | aaa | A | A* |
| 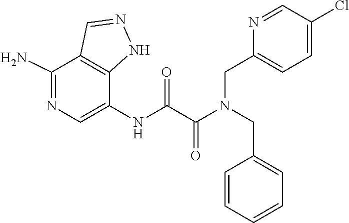 [SMILES: Nc1ncc(NC(=O)C(=O)N(Cc2ccccc2)Cc2ccc(Cl)cn2)c2[nH]ncc12] | 158 | | b | aa | aaa | C | A* |
| 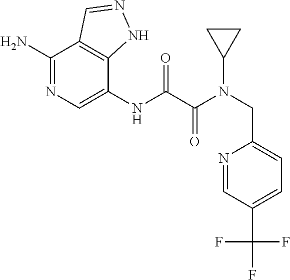 [SMILES: Nc1ncc(NC(=O)C(=O)N(Cc2ccc(C(F)(F)F)cn2)C2CC2)c2[nH]ncc12] | 159 | | c | aa | bbb | B | A* |
| 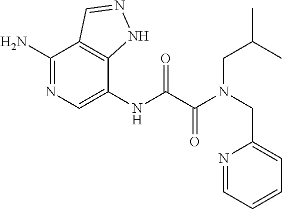 [SMILES: CC(C)CN(Cc1ccccn1)C(=O)C(=O)Nc1cnc(N)c2cn[nH]c12] | 160 | | c | aa | bbb | A | B* |
| 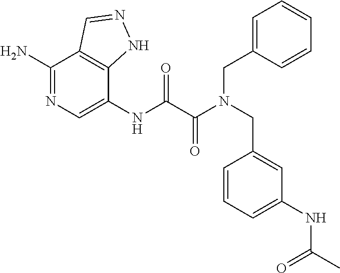 [SMILES: CC(=O)Nc1cccc(CN(Cc2ccccc2)C(=O)C(=O)Nc2cnc(N)c3cn[nH]c23)c1] | 161 | | c | aa | aaa | A | A* |
| 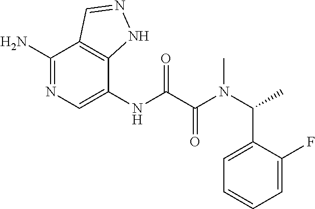 [SMILES: C[C@H](c1ccccc1F)N(C)C(=O)C(=O)Nc1cnc(N)c2cn[nH]c12] | 162 | (abs) | c | bb | bbb | A | B* |
| 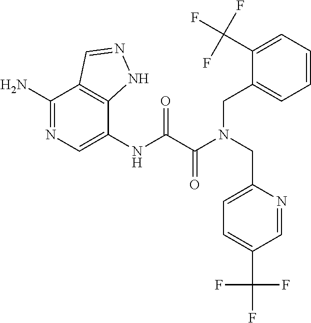 [SMILES: Nc1ncc(NC(=O)C(=O)N(Cc2ccc(C(F)(F)F)cn2)Cc2ccccc2C(F)(F)F)c2[nH]ncc12] | 163 | | b | bb | bbb | C | A* |
| 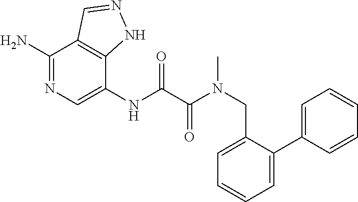 [SMILES: CN(Cc1ccccc1-c1ccccc1)C(=O)C(=O)Nc1cnc(N)c2cn[nH]c12] | 164 | | c | aa | ccc | A | B* |
| 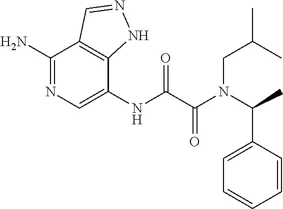 [SMILES: CC(C)CN(C(=O)C(=O)Nc1cnc(N)c2cn[nH]c12)[C@@H](C)c1ccccc1] | 165 | (abs) | c | bb | bbb | A | A* |
| 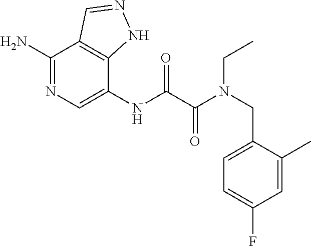 [SMILES: CCN(Cc1ccc(F)cc1C)C(=O)C(=O)Nc1cnc(N)c2cn[nH]c12] | 166 | | c | aa | ccc | A | A* |
| 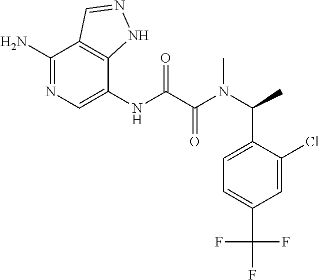 [SMILES: C[C@@H](c1ccc(C(F)(F)F)cc1Cl)N(C)C(=O)C(=O)Nc1cnc(N)c2cn[nH]c12] | 167 | (abs) | c | aa | aaa | A | A* |
| 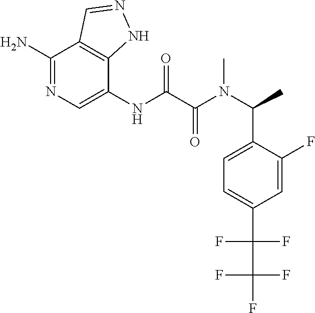 [SMILES: C[C@@H](c1ccc(C(F)(F)C(F)(F)F)cc1F)N(C)C(=O)C(=O)Nc1cnc(N)c2cn[nH]c12] | 168 | (abs) | c | bb | bbb | A | A* |
| 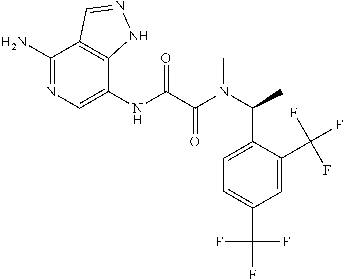 [SMILES: C[C@@H](c1ccc(C(F)(F)F)cc1C(F)(F)F)N(C)C(=O)C(=O)Nc1cnc(N)c2cn[nH]c12] | 169 | (abs) | c | bb | ccc | A | B* |
| 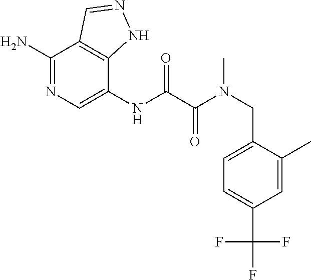 [SMILES: Cc1cc(C(F)(F)F)ccc1CN(C)C(=O)C(=O)Nc1cnc(N)c2cn[nH]c12] | 170 | | c | aa | aaa | A | A* |
| 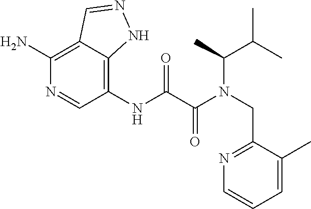 [SMILES: Cc1cccnc1CN(C(=O)C(=O)Nc1cnc(N)c2cn[nH]c12)[C@@H](C)C(C)C] | 171 | (or) | b | aa | aaa | C | A* |
| 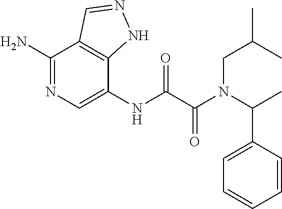 [SMILES: CC(C)CN(C(=O)C(=O)Nc1cnc(N)c2cn[nH]c12)C(C)c1ccccc1] | 172 | | c | aa | aaa | B | A* |
| 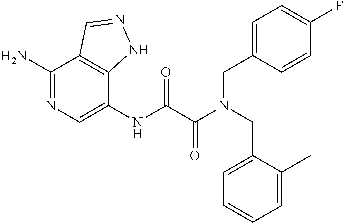 [SMILES: Cc1ccccc1CN(Cc1ccc(F)cc1)C(=O)C(=O)Nc1cnc(N)c2cn[nH]c12] | 173 | | b | aa | aaa | C | A* |
| 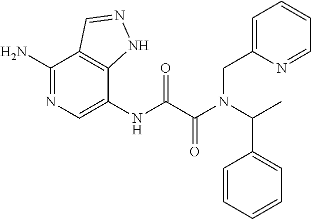 [SMILES: CC(c1ccccc1)N(Cc1ccccn1)C(=O)C(=O)Nc1cnc(N)c2cn[nH]c12] | 174 | | b | aa | aaa | B | A* |
| 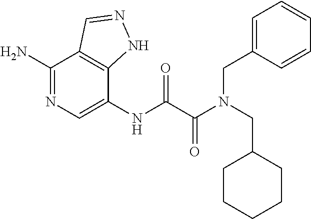 [SMILES: Nc1ncc(NC(=O)C(=O)N(Cc2ccccc2)CC2CCCCC2)c2[nH]ncc12] | 175 | | c | aa | aaa | A | A* |
| 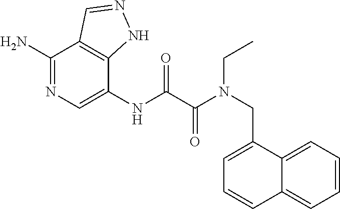 [SMILES: CCN(Cc1cccc2ccccc12)C(=O)C(=O)Nc1cnc(N)c2cn[nH]c12] | 176 | | c | bb | ccc | A | B* |
| 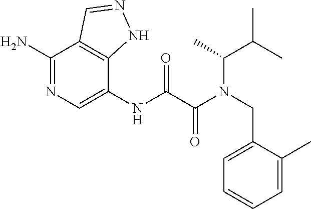 [SMILES: Cc1ccccc1CN(C(=O)C(=O)Nc1cnc(N)c2cn[nH]c12)[C@H](C)C(C)C] | 177 | (abs) | c | aa | aaa | B | A* |
| 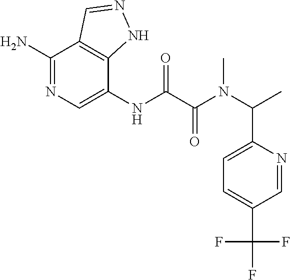 [SMILES: CC(c1ccc(C(F)(F)F)cn1)N(C)C(=O)C(=O)Nc1cnc(N)c2cn[nH]c12] | 178 | | c | aa | aaa | A | B* |
| 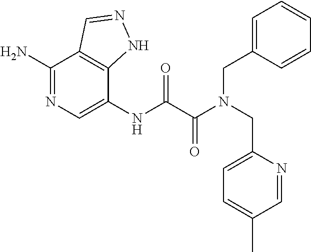 [SMILES: Cc1ccc(CN(Cc2ccccc2)C(=O)C(=O)Nc2cnc(N)c3cn[nH]c23)nc1] | 179 | | b | aa | aaa | A | A* |
| 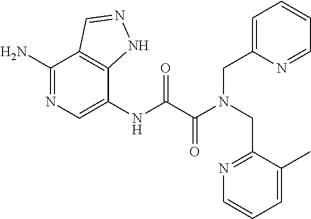 [SMILES: Cc1cccnc1CN(Cc1ccccn1)C(=O)C(=O)Nc1cnc(N)c2cn[nH]c12] | 180 | | b | aa | aaa | C | A* |
| 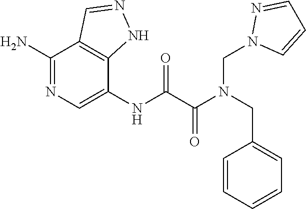 [SMILES: Nc1ncc(NC(=O)C(=O)N(Cc2ccccc2)Cn2cccn2)c2[nH]ncc12] | 181 | | c | aa | aaa | A | B* |
| 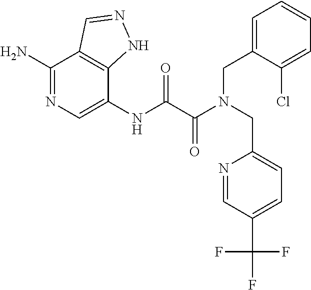 [SMILES: Nc1ncc(NC(=O)C(=O)N(Cc2ccc(C(F)(F)F)cn2)Cc2ccccc2Cl)c2[nH]ncc12] | 182 | | b | bb | aaa | C | A* |
| 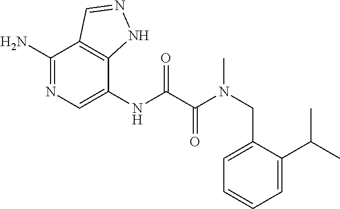 [SMILES: CC(C)c1ccccc1CN(C)C(=O)C(=O)Nc1cnc(N)c2cn[nH]c12] | 183 | | c | bb | ccc | A | B* |
| 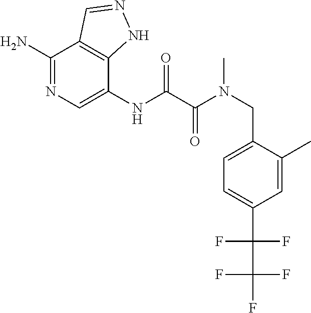 [SMILES: Cc1cc(C(F)(F)C(F)(F)F)ccc1CN(C)C(=O)C(=O)Nc1cnc(N)c2cn[nH]c12] | 184 | | c | aa | aaa | A | A* |
| 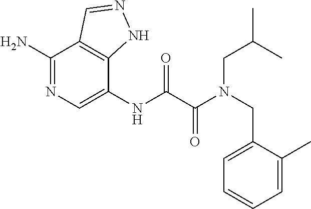 [SMILES: Cc1ccccc1CN(CC(C)C)C(=O)C(=O)Nc1cnc(N)c2cn[nH]c12] | 185 | | c | aa | aaa | A | A* |
| 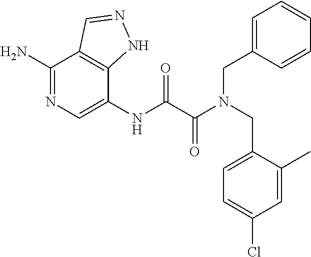 [SMILES: Cc1cc(Cl)ccc1CN(Cc1ccccc1)C(=O)C(=O)Nc1cnc(N)c2cn[nH]c12] | 186 | | b | aa | aaa | B | A* |
| 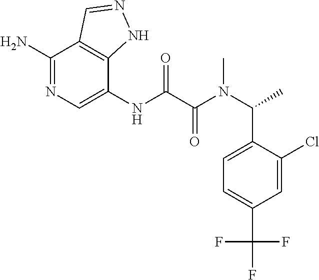 [SMILES: C[C@H](c1ccc(C(F)(F)F)cc1Cl)N(C)C(=O)C(=O)Nc1cnc(N)c2cn[nH]c12] | 187 | (abs) | b | aa | aaa | A | A* |
| 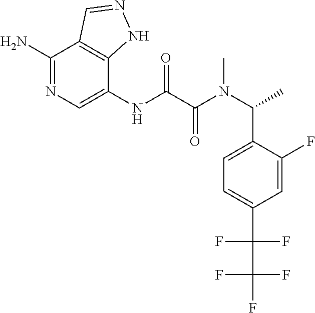 [SMILES: C[C@H](c1ccc(C(F)(F)C(F)(F)F)cc1F)N(C)C(=O)C(=O)Nc1cnc(N)c2cn[nH]c12] | 188 | (abs) | b | aa | aaa | B | A* |
| 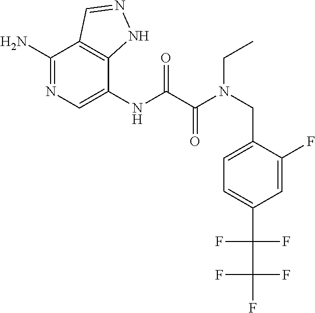 [SMILES: CCN(Cc1ccc(C(F)(F)C(F)(F)F)cc1F)C(=O)C(=O)Nc1cnc(N)c2cn[nH]c12] | 189 | | c | aa | aaa | B | A* |
| 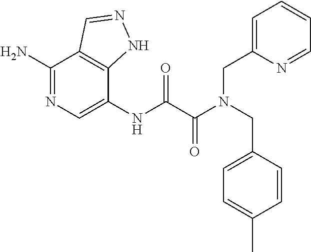 [SMILES: Cc1ccc(CN(Cc2ccccn2)C(=O)C(=O)Nc2cnc(N)c3cn[nH]c23)cc1] | 190 | | b | aa | aaa | A | A* |
| 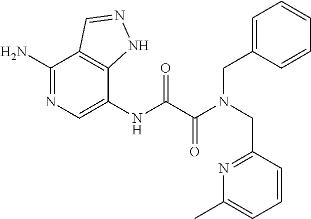 [SMILES: Cc1cccc(CN(Cc2ccccc2)C(=O)C(=O)Nc2cnc(N)c3cn[nH]c23)n1] | 191 | | b | aa | aaa | A | A* |
| 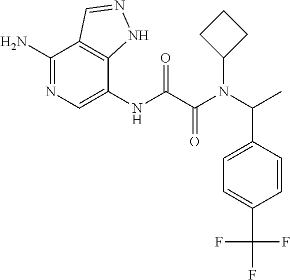 [SMILES: CC(c1ccc(C(F)(F)F)cc1)N(C(=O)C(=O)Nc1cnc(N)c2cn[nH]c12)C1CCC1] | 192 | | b | aa | aaa | B | A* |
| 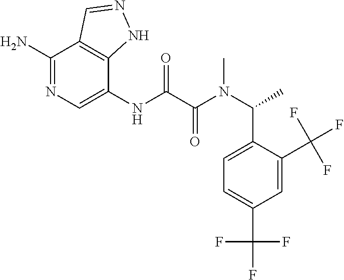 [SMILES: C[C@H](c1ccc(C(F)(F)F)cc1C(F)(F)F)N(C)C(=O)C(=O)Nc1cnc(N)c2cn[nH]c12] | 193 | (abs) | c | aa | aaa | A | A* |
| 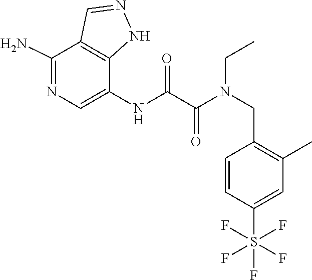 [SMILES: CCN(Cc1ccc(S(F)(F)(F)(F)F)cc1C)C(=O)C(=O)Nc1cnc(N)c2cn[nH]c12] | 194 | | b | aa | aaa | B | A* |
| 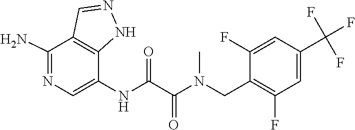 [SMILES: CN(Cc1c(F)cc(C(F)(F)F)cc1F)C(=O)C(=O)Nc1cnc(N)c2cn[nH]c12] | 195 | | C | aa | aaa | A | A* |
| 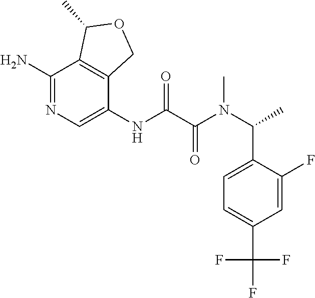 [SMILES: C[C@@H]1OCc2c(NC(=O)C(=O)N(C)[C@H](C)c3ccc(C(F)(F)F)cc3F)cnc(N)c21] | 196 | (abs) on benzyl, (or) on tetra- hydro- furan | | | | | B* |
| 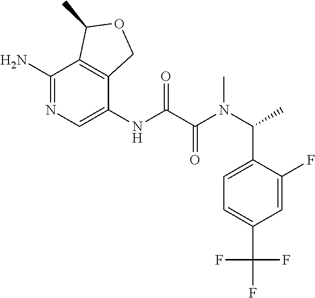 [SMILES: C[C@H](c1ccc(C(F)(F)F)cc1F)N(C)C(=O)C(=O)Nc1cnc(N)c2c1CO[C@@H]2C] | 197 | (abs) on benzyl, (or) on tetra- hydro- furan | | | | | C* |

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In another embodiment, provided is a pharmaceutical composition comprising a pharmaceutically acceptable carrier and an effective amount of a compound described herein (e.g., a compound of Formula (A) or a compound of Table 1), or a pharmaceutically acceptable salt thereof.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat. Cyclodextrins such as α-, β-, and γ-cyclodextrin, or chemically modified derivatives such as hydroxyalkylcyclodextrins, including 2 and 3 hydroxypropyl-β-cyclodextrins, or other solubilized derivatives may also be advantageously used to enhance delivery of compounds of the formulae described herein.

When employed as pharmaceuticals, the compounds provided herein are typically administered in the form of a pharmaceutical composition. Such compositions can be prepared in a manner well known in the pharmaceutical art and comprise at least one active compound.

In one embodiment, with respect to the pharmaceutical composition, the carrier is a parenteral carrier, oral or topical carrier.

Also provided is a compound described herein (e.g., a compound of Formula (A) or a compound of Table 1, or pharmaceutically acceptable salts thereof)(or pharmaceutical composition thereof) for use as a pharmaceutical or a medicament (e.g., a medicament for the treatment of an MTAP-deficient and/or an MTA-accumulating disease in a subject in need thereof). In one embodiment, the disease is a proliferating disease. In a further embodiment, the disease is an MTAP-deficient and/or MTA-accumulating cancer. In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Also provided is a compound described herein (e.g., a compound of Formula (A) or a compound of Table 1, or pharmaceutically acceptable salts thereof)(or pharmaceutical composition thereof) for use in the treatment of an MTAP-deficient and/or an MTA-accumulating disease in a subject in need thereof. In one embodiment, the disease is a proliferating disease. In a further embodiment, the disease is an MTAP-deficient and/or MTA-accumulating cancer. In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Also provided is a compound described herein (e.g., a compound of Formula (A) or a compound of Table 1, or pharmaceutically acceptable salts thereof)(or pharmaceutical composition thereof) for use in the manufacturing of a medicament (e.g., a medicament for the treatment of an MTAP-deficient and/or an MTA-accumulating disease in a subject in need thereof). In one embodiment, the disease is a proliferating disease. In a further embodiment, the disease is an MTAP-deficient and/or MTA-accumulating cancer. In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Generally, the compounds provided herein are administered in an effective amount (e.g., a therapeutically effective amount). The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional nontoxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The pharmaceutical compositions provided herewith may also be administered in the form of suppositories for rectal administration. These compositions can be prepared by mixing a compound provided herewith with a suitable non irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions provided herewith may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The above-described components for orally administrable, injectable or topically administrable, rectally administrable and nasally administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds described herein can also be administered in sustained release forms or from sustained release drug delivery systems. A description of representative sustained release materials can be found in Remington's Pharmaceutical Sciences.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

Also provided is the pharmaceutically acceptable acid addition salt of a compound described herein (e.g., compound of Formula (A) or a compound of Table 1).

The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

### Methods of Treatment and Use

Treatment of MTAP-Deficient and/or MTA-Accumulating Proliferation Disorders

5-Methylthioadenosine phosphorylase (MTAP) catalyzes the reversible phosphorylation of S-methyl-5’-thioadenosine (MTA) to adenine and 5-methylthioribose-1-phosphate. MTAP-deletion is a common genetic event in human cancer. MTAP deletion frequency in a subset of human cancers is described in Cerami et al., Cancer Discov. (2012);2 (5): 401-4; Gao et al., Sci Signal. (2013);6 (269): pl1; and Lee et al., Nat. Gen. (2014) 46 (11): 1227-32. For example, more than 50% of malignant peripheral nerve sheath tumor (MPNST) have deletions in MTAP(Lee et al., Nat. Gen. (2014)). Other cancers with high MTAP deletion frequencies are glioblastoma (GBM), mesothelioma, bladder cancer, pancreatic cancer, esophageal cancer, squamous lung cancer, melanoma, diffuse large B cell lymphoma (DLBCL), head and neck cancer, cholangiocarcinoma, lung adenoma, sarcoma, stomach cancer, glioma, adrenal carcinoma, thymoma, breast cancer, liver cancer, ovarian cancer, renal papillary cancer, uterine cancer, prostate cancer, and renal clear cell cancer. MTAP deletion in cells is one of the mechanisms that leads to MTAP-deficiency, increased intracellular MTA accumulation, and confers enhanced dependency on the protein arginine methyltransferase 5 (PRMT5) in cancer cells. Other mechanisms leading to MTAP deficiency include, inter alia, MTAP translocations and MTAP epigenetic silencing which could also lead to MTAP-null and/or MTAP deficient tumors. PRMT5 mediates the formation of symmetric dimethylarginine (SDMA); thus, the PRMT5 activity can be assessed by measuring the SDMA levels using the antibody against an SDMA or SDMA modified polypeptide.

In one embodiment, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of a compound of the present disclosure (e.g., a compound of Formula (A) or a compound of Table 1) or a pharmaceutically acceptable salt thereof.

In some embodiments, provided is a compound of the present disclosure (e.g., a compound of Formula (A) or a compound of Table 1), or a pharmaceutical composition comprising a compound of formula (A) of the present disclosure for use in a method of treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a compound of the present disclosure (e.g., a compound of Formula (A) or a compound of Table 1), or a pharmaceutical composition comprising a compound of formula (A) of the present disclosure for use in the manufacturing of a medicament for treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a use of a compound of the present disclosure (e.g., a compound of Formula (A) or a compound of Table 1), or of a pharmaceutical composition comprising a compound of formula (A) of the present disclosure in a method of treating human or animal subjects having or having been diagnosed with an MTAP-deficiency-related and/or MTA-accumulating proliferative disorder (e.g., cancer). In some embodiments, the use is of a therapeutically effective amount of the compound or composition.

In some embodiments, provided is use of a compound of the present disclosure (e.g., a compound of Formula (A) or a compound of Table 1), or of a pharmaceutical composition comprising a compound of formula (A) of the present disclosure in the manufacturing of a medicament for treating human or animal subjects having or having been diagnosed with an MTAP-deficiency-related and/or MTA-accumulating proliferative disorder (e.g., cancer). In some embodiments, the use is of a therapeutically effective amount of the compound or composition.

In one embodiment, provided are methods for treating an MTAP-deficiency-related and/or MTA-accumulating proliferative disorder (e.g., cancer) in a subject in need thereof comprising administering to the subject an effective amount (e.g., a therapeutically effective amount) of a compound of the present disclosure (e.g., compound of Formula (A) or a compound of Table 1) or a pharmaceutically acceptable salt thereof.

In one embodiment, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of pharmaceutical composition of the present disclosure (e.g., a composition comprising a compound of Formula (A) or a compound of Table 1, or pharmaceutically acceptable salts thereof and a pharmaceutically acceptable carrier). In one embodiment, the compound or composition is administered in combination with a second therapeutic agent.

In one embodiment, provided are methods of treating an MTAP-deficiency-related and/or MTA-accumulating proliferative disorder (e.g., cancer) in a subject in need thereof comprising administering to the subject a therapeutically effective amount of pharmaceutical composition of the present disclosure (e.g., a composition comprising a compound of Formula (A) or a compound of Table 1, or pharmaceutically acceptable salts thereof and a pharmaceutically acceptable carrier). In one embodiment, the compound or composition is administered in combination with a second therapeutic agent.

In some embodiments, the subject is human.

In certain embodiments, the disease is an MTAP-deficient and/or MTA-accumulating cancer.

In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment, the cancer is an MTAP-deficient and/or MTA-accumulating glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive or mixed mode PRMT5 inhibitor or an MTA cooperative binding agent, e.g., a compound of Formula (A) or a compound of Table 1, or pharmaceutically acceptable salts thereof) described herein can be used in a method of inhibiting proliferation of MTAP-deficient cells in a subject in need thereof, the method comprising the step of administering to the subject, a PRMT5 inhibitor (e.g., an MTA-uncompetitive, non-competitive or mixed mode PRMT5 inhibitor or an MTA cooperative binding agent, e.g., a compound of Formula (A) or a compound of Table 1, or pharmaceutically acceptable salts thereof) in an amount that is effective to inhibit proliferation of the MTAP-deficient cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive or mixed mode PRMT5 inhibitor or an MTA cooperative binding agent, e.g., a compound of Formula (A) or a compound of Table 1, or pharmaceutically acceptable salts thereof) described herein can be used in a method of inhibiting proliferation of MTA-accumulating cells in a subject in need thereof, the method comprising the step of administering to the subject, a PRMT5 inhibitor (e.g., an MTA-uncompetitive, non-competitive or mixed mode PRMT5 inhibitor or an MTA cooperative binding agent, e.g., a compound of Formula (A) or a compound of Table 1, or pharmaceutically acceptable salts thereof) in an amount that is effective to inhibit proliferation of the MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof) described herein can be used in a method of inhibiting proliferation of MTAP deficient and/or MTA-accumulating cells in a subject in need thereof, the method comprising the step of administering to the subject, a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof) in an amount that is effective to inhibit proliferation of the MTAP deficient and/or MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

### Combination Therapies

In some embodiments, provided are methods of treatment of MTAP-deficient and/or MTA accumulating proliferative disorders (e.g., cancers) with a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof) in combination with one or more therapeutic agent.

In some embodiments, provided are methods of treatment of MTAP-deficient and/or MTA accumulating proliferative disorders (e.g., cancers) with a PRMT5 inhibitor described herein (e.g., a compound of Formula (A) or a compound of Table 1, or pharmaceutically acceptable salts thereof) in combination with a second therapeutic agent. In some embodiments, provided are methods of treatment of MTAP-deficient and/or MTA accumulating proliferative disorders (e.g., cancers) with PRMT5 inhibitor described herein (e.g., a compound of Formula (A) or a compound of Table 1, or pharmaceutically acceptable salts thereof) in combination with a second therapeutic agent and a third therapeutic agent. In some embodiments, provided are methods of treatment of MTAP-deficient and/or MTA accumulating proliferative disorders (e.g., cancers) with PRMT5 inhibitor described herein (e.g., a compound of Formula (A) or a compound of Table 1, or pharmaceutically acceptable salts thereof) in combination with a second therapeutic agent, a third therapeutic agent, and a fourth therapeutic agent.

The term “Combination” refers to either a fixed combination in one dosage unit form, or a combined administration where a PRMT5 inhibitor described herein (e.g., a compound of Formula (A) or a compound of Table 1, or pharmaceutically acceptable salts thereof) and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., PRMT5 inhibitors described herein (e.g., a compound of Formula (A) or a compound of Table 1, or pharmaceutically acceptable salts thereof) and a combination partner, are both administered to a patient simultaneously in the form of a single entity or dosage. The term “non-fixed combination” means that the therapeutic agents, e.g., a PRMT5 inhibitor described herein (e.g., a compound of Formula (A) or a compound of Table 1, or pharmaceutically acceptable salts thereof) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In certain embodiments, PRMT5 inhibitors described herein are combined with other therapeutic agents, including, but not limited to, other anti-cancer agents, anti-allergic agents, anti-nausea agents (or anti-emetics), pain relievers, cytoprotective agents, and combinations thereof.

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and a general chemotherapeutic agents selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and an EGFR-inhibitor (e.g., cetuximab, panitumimab, erlotinib, gefitinib and EGFRi NOS). In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and a MAPK-pathway inhibitor (e.g., BRAFi, panRAFi, MEKi, ERKi). In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and a PI3K-mTOR pathway inhibitor (e.g., alpha-specific PI3Ki, pan-class I PI3Ki and mTOR/PI3Ki, particularly everolimus and analogues thereof).

MTAP-deletion can co-occur with mutations in the KRAS gene (e.g., KRASG12C). In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof), and a KRAS inhibitor (e.g., a pan-KRAS or a specific G12C, G12D, G13C inhibitor, e.g., adagrasib, sotorasib, LY3537982, RMC-6236, RMC-6291, RMC-9805, RMC-8839).

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof), and a Spliceosome inhibitor (e.g., SF3b1 inhibitors; e.g., E7107).

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and an HDAC inhibitor or DNA methyltransferase inhibitor. In some embodiments, the HDAC inhibitor is Trichostatin A. In some embodiments, the DNA methyltransferase inhibitor is 5-azacytidine.

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and a MAT2A inhibitor.

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and an inhibitor of a protein which interacts with or is required for PRMT5 function, including, but not limited to, pICIN, WDR77 or RIOK1.

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and an HDM2 inhibitor and/or 5-FU or other purine analogues (e.g., 6-thioguanine, 6-mercaptopurine).

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and a CDK4 inhibitor, including, but not limited to, LEE011 or a CDK 4/6 inhibitor (e.g., palbociclib (Ibrance®), ribociclib (Kisqali®), and abemaciclib (Verzenio®).

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and a targeted treatment contingent on the dependency of individual target tumors on relevant pathways as determined by suitable predictive markers, including but not limited to: inhibitors of HDM2i, PI3K/mTOR-I, MAPKi, RTKi (EGFRi, FGFRi, METi, IGFiRi, JAKi, and WNTi.

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and immunotherapy.

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and an immunotherapeutic agent.

In some embodiments, the immunotherapeutic agent is an anti-CTLA-4 antibody (e.g., ipilimumab, tremelimumab).

In some embodiments, the immunotherapeutic agent is an anti-PD-1 or anti-PD-L1 agent (e.g., an antibody). In some embodiments, the immunotherapeutic agent is an anti-PD-1 agent (e.g., an anti-PD-1 antibody, e.g., nivolumab (i.e., MDX-1106, BMS-936558, ONO-4538); CT-011; AMP-224; pembrolizumab (MK-3475); pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab). In some embodiments, the immunotherapeutic agent is an anti-PD-L1 agent (e.g., an anti-PD-L1 antibody, e.g., BMS936559 (i.e., MDX-1105); durvalumab (MEDI4736); avelumab (MSB0010718C); envafolimab; cosibelimab; sugemalimab, AUNP-12 or atezolizumab (MPDL-3280A) or an anti-PD-L1 small molecule (e.g., CA-170)).

In some embodiments, the immunotherapeutic agent is a checkpoint blocking antibody (e.g., anti-TIM3, anti-LAG3, anti-TIGIT including IMP321 and MGA271).

In some embodiments, the immunotherapeutic agent is a cell-based therapy. In some embodiments, the cell-based therapy is a CAR-T therapy.

In some embodiments, the immunotherapeutic agent is a co-stimulatory antibody (e.g., anti-4-1BB, anti-OX40, anti-GITR, anti-CD27, anti-CD40).

In some embodiments, the immunotherapeutic agent is a cancer vaccine such as a neoantigen. These vaccines can be developed using peptides or RNA (e.g., mRNA).

In some embodiments, the immunotherapeutic agent is an oncolytic virus.

In some embodiments, the immunotherapeutic agent is a STING pathway agonist. Exemplary STING agonists include MK-1454 and ADU-S100.

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and a disease-specific huMAB (e.g., an anti-HER³ huMAB).

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and an ADC/ADCC contingent on the expression of relevant surface targets on target tumors of interest.

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and one or more DNA damage pathway inhibitor. In some embodiments, a DNA damage pathway inhibitor is selected from the group consisting of bleomycin, an ATM inhibitor (e.g., AZD1390), a USP1 inhibitor, a WEE1 inhibitor (e.g., AZD1775), and a Chk1 inhibitor (e.g., AZD7762). In some embodiments, a DNA damage pathway inhibitor is a DNA alkylating agent.

In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and a PARP inhibitor. In some embodiments, a PARP inhibitor is selected from the group consisting of olaparib, rucaparib, niraparib, talazoparib, veliparib, pamiparib, CEP 9722, E7016, iniparib, and 3-aminobenzamide.

Some patients may experience allergic reactions to the PRMT5 inhibitors described herein and/or other anti-cancer agent(s) during or after administration; therefore, anti-allergic agents are often administered to minimize the risk of an allergic reaction. In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and an anti-allergic agent (e.g., a corticosteroid, including, but not limited to, dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); an antihistamine, such as diphenhydramine (e.g., Benadryl®), hydroxyzine, and cyproheptadine; a bronchodilator, such as the beta-adrenergic receptor agonists, albuterol (e.g., Proventil®), and terbutaline (Brethine®)

Some patients may experience nausea during and after administration of the PRMT5 inhibitors described herein and/or other anti-cancer agent(s); therefore, anti-emetics are used in preventing nausea (upper stomach) and vomiting. In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and an anti-emetic (e.g., aprepitant (Emend®), ondansetron (Zofran®), granisetron HCl (Kytril®), lorazepam (Ativan®), dexamethasone (Decadron®), prochlorperazine (Compazine®), casopitant (Rezonic® and Zunrisa®), and combinations thereof).

Medication to alleviate the pain experienced during the treatment period is often prescribed to make the patient more comfortable. In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and an analgesic (e.g., an over-the-counter analgesic (e.g., Tylenol®), an opioid analgesic (e.g., hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), fentanyl (e.g., Duragesic®))).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and a cytoprotective agent (e.g., Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid)).

The structure of the active compounds identified by code numbers, generic or trade names may be taken from the actual edition of the standard compendium “The Merck Index” or from databases, e.g., Patents International (e.g., IMS World Publications).

The above-mentioned compounds, which can be used in combination with a PRMT5 inhibitor as described herein, can be prepared and administered as described in the art, including, but not limited to, in the documents cited above.

In one embodiment, provided are pharmaceutical compositions comprising at least one compound of the present disclosure (e.g., a PRMT5 inhibitor, e.g., a compound of Formula (A) or a compound of Table 1) or a pharmaceutically acceptable salt thereof together with a pharmaceutically acceptable carrier suitable for administration to a human or animal subject, either alone or together with other anti-cancer agents.

In one embodiment, provided are methods of treating human or animal subjects having or having been diagnosed with an MTAP-deficient and/or MTA accumulating proliferative disorder (e.g., cancer) comprising administering to the subject in need thereof a pharmaceutical composition comprising a therapeutically effective amount of a compound of the present disclosure (e.g., a compound of Formula (A) or a compound of Table 1) or a pharmaceutically acceptable salt thereof in combination with one or more therapeutic agents as described herein.

In one embodiment, provided are methods of treating an MTAP-deficient and/or MTA accumulating proliferative disorder (e.g., cancer) in a subject in need thereof comprising administering to the subject a pharmaceutical composition comprising an effective amount (e.g., a therapeutically effective amount) of a compound of the present disclosure (e.g., a compound of Formula (A) or a compound of Table 1) or a pharmaceutically acceptable salt thereof in combination with one or more therapeutic agents as described herein.

In particular, compositions will either be formulated together as a combination therapeutic or administered separately.

In combination therapy, a PRMT5 inhibitor as described herein and other anti-cancer agent(s) may be administered either simultaneously, concurrently or sequentially with no specific time limits, wherein such administration provides therapeutically effective levels of the two compounds in the body of the patient.

In a preferred embodiment, the compound of the present disclosure (e.g., a compound of Formula (A) or a compound of Table 1, or pharmaceutically acceptable salts thereof) and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The PRMT5 inhibitor as described herein and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another embodiment, provided are kits that include one or more PRMT5 inhibitor(s) as described herein (e.g., a compound of Formula (A) or a compound of Table 1, or pharmaceutically acceptable salts thereof) and a second therapeutic agent as disclosed herein are provided. Representative kits include (a) a PRMT5 inhibitor as described herein or a pharmaceutically acceptable salt thereof (e.g., a compound of Formula (A) or a compound of Table 1, or pharmaceutically acceptable salts thereof), (b) at least one other therapeutic agent, e.g., as indicated above, whereby such kit may comprise a package insert or other labeling including directions for administration.

A PRMT5 inhibitor as described herein (e.g., a compound of Formula (A) or a compound of Table 1, or pharmaceutically acceptable salts thereof) may also be used in combination with known therapeutic processes, for example, the administration of hormones or especially radiation. A compound of the present disclosure may in particular be used as a radiosensitizer, especially for the treatment of tumors which exhibit poor sensitivity to radiotherapy. In some embodiments, provided is a method of treating a disease or disorder (e.g., cancer) comprising administering or coadministering, in any order, to a patient in need thereof a PRMT5 inhibitor described herein (e.g., a compound of Formula (A) or a compound of Table 1, or pharmaceutically acceptable salts thereof) and radiation.

### Patient Selection and Monitoring

In one embodiment, provided is a method of determining if a subject having or having been diagnosed with a cancer (e.g., a cancer patient) will respond to therapeutic treatment with a PRMT5 inhibitor (e.g., an MTA-uncompetitive PRMT5 inhibitor, e.g., a compound of Formula (A) or a compound of Table 1, or pharmaceutically acceptable salts thereof), comprising the steps of:
a) contacting a test sample obtained from said subject with a reagent capable of detecting human cancer cells that have MTAP deficiency and/or MTA accumulation; and
b) comparing the test sample with a reference (e.g., a reference sample taken from a non-cancerous or normal control subject),
wherein the presence of MTAP deficiency and/or MTA accumulation in said test sample indicates that the subject will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof).

In one embodiment, provided is a method of determining if a cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof), comprising the steps of:
a) contacting a test sample obtained from a subject having or having been diagnosed with said cancer with a reagent capable of detecting human cancer cells that have MTAP deficiency and/or MTA accumulation; and
b) comparing the test sample with a reference (e.g., a reference sample taken from a non-cancerous or normal control subject),
   wherein the presence of MTAP deficiency and/or MTA accumulation in said test sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof). In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma. In some embodiments, the method further comprises the step of determining the level of PRMT5 in the cancer cells. The level of expression of PRMT5 can be considered when determining the therapeutically effective dosage of a PRMT5 inhibitor.

In one embodiment, provided is a method of determining the sensitivity of a cancer cell to PRMT5 inhibition (e.g., inhibition with an MTA-uncompetitive PRMT5 inhibitor, e.g., a compound of Formula (A) or a compound of Table 1, or pharmaceutically acceptable salts thereof), comprising the steps of:
a) assaying the production, level, activity, expression or presence of MTAP), in said cancer cell;
b) comparing the production, level, activity, expression or presence of MTAP in the cancer cell with the production, level, activity, expression or presence of MTAP, respectively, in a non-cancerous or normal control cell,
   wherein a decreased level, activity or expression in the cancer cell indicates MTAP deficiency and wherein MTAP deficiency indicates that said cancer cell is sensitive to the PRMT5 inhibitor.

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment, provided is a method of determining the sensitivity of a cancer cell to a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof), comprising the steps of:
a) assaying for level, activity or expression of the MTAP gene or its gene product in both the cancer cell and a normal control cell, wherein a decreased level, activity or expression in the cancer cell indicates MTAP deficiency; b) assaying for PRMT5 expression in said cancer cell; c) comparing the PRMT5 expression with PRMT5 expression in the cancer cell and a normal control cell; wherein the similarity in PRMT5 expression, and the presence of said MTAP deficiency in said cancer cell, indicates said cell is sensitive to a PRMT5 inhibitor.

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment the provided is a therapeutic method of treating a subject having or having been diagnosed with a cancer (e.g., a cancer associated with MTAP deficiency and/or MTA accumulation) comprising the steps of:
a) assessing the level of MTAP and/or MTA in a test sample obtained from said subject (e.g., by contacting the sample with a reagent capable of detecting human MTAP-deficient and/or MTA-accumulating cancer cells in a test sample obtained from said subject), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor; and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof) to the subject identified in step b).

In one embodiment provided is a therapeutic method of treating a cancer (e.g., a cancer associated with MTAP deficiency and/or MTA accumulation) in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof) to the subject identified in step b).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

In one embodiment provided is a therapeutic method of treating a subject having or having been diagnosed with a cancer associated with MTAP deficiency and/or MTA accumulation comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof) to the subject identified in step b).

In one embodiment provided is a therapeutic method of treating cancer associated with MTAP deficiency and/or MTA accumulation in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive PRMT5 inhibitor e.g., a compound of Formula (A) or a compound of Table 1, or pharmaceutically acceptable salts thereof) to the subject identified in step b).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

In one embodiment provided is a method of determining if a subject having or having been diagnosed with a cancer associated with MTAP deficiency and/or MTA accumulation will respond to treatment with a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof) comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor (e.g., an MTA-uncompetitive, non-competitive, or mixed mode PRMT5 inhibitor or an MTA-cooperative binding agent).

In one embodiment provided is a method of determining if a cancer associated with MTAP deficiency and/or MTA accumulation will respond to treatment with a PRMT5 inhibitor (e.g., an MTA-uncompetitive PRMT5 inhibitor, e.g., a compound of Formula (A) or a compound of Table 1, or pharmaceutically acceptable salts thereof) comprising the steps of:
a) assessing the level of MTAP and/or MTA in a test sample obtained from a subject having or having been diagnosed with said cancer (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

### Sample Preparation

Further Provided are Assays for the Detection of MTAP Deficiency and/or MTA accumulation. They can include detecting a mutation related to MTAP deficiency and/or MTA accumulation, e.g., in a body fluid such as blood (e.g., serum or plasma) bone marrow, cerebral spinal fluid, peritoneal/pleural fluid, lymph fluid, ascites, serous fluid, sputum, lacrimal fluid, stool, and urine, or in a tissue such as a tumor tissue. The tumor tissue can be fresh tissue or preserved tissue (e.g., formalin fixed tissue, e.g., paraffin-embedded tissue).

Body fluid samples can be obtained from a subject using any of the methods known in the art. Methods for extracting cellular DNA from body fluid samples are well known in the art. Typically, cells are lysed with detergents. After cell lysis, proteins are removed from DNA using various proteases. DNA is then extracted with phenol, precipitated in alcohol, and dissolved in an aqueous solution. Methods for extracting acellular DNA from body fluid samples are also known in the art. Commonly, a cellular DNA in a body fluid sample is separated from cells, precipitated in alcohol, and dissolved in an aqueous solution.

### Detection of PRMT5 Selectivity

Samples, once prepared, can be tested for MTAP deficiency and/or MTA accumulation, either or both of which indicates that the sample is sensitive to treatment with a PRMT5 inhibitor. Cells can be determined to be MTA accumulating by techniques known in the art; methods for detecting MTA include, as a non-limiting example, liquid chromatography-electrospray ionization-tandem mass spectrometry (LC-ESI-MS/MS), as described in Stevens et al. 2010. J. Chromatogr. A. 1217:3282-3288; and Kirovski et al. 2011 Am. J. Pathol. 178:1145-1152; and references cited therein. The detection of MTAP deficiency can be done by any number of ways, for example: DNA sequencing, PCR based methods, including RT-PCR, microarray analysis, Southern blotting, Northern blotting, Next Generation Sequencing, and dip stick analysis. In some embodiments, MTAP deficiency is evaluated by any technique known in the art, for example, immunohistochemistry utilizing an anti-MTAP antibody or derivative thereof, and/or genomic sequencing, or nucleic acid hybridization, or amplification utilizing at least one probe or primer comprising a sequence of at least 12 contiguous nucleotides (nt) of the sequence of MTAP wherein the primer is no longer than about 30 nt.

The polymerase chain reaction (PCR) can be used to amplify and identify MTAP deficiency from either genomic DNA or RNA extracted from tumor tissue. PCR is well known in the art and is described in detail in Saiki et al., Science 1988, 239:487.

Methods of detecting MTAP deficiency by hybridization are provided. The method comprises identifying MTAP deficiency in a sample by its inability to hybridize to MTAP nucleic acid. The nucleic acid probe is detectably labeled with a label such as a radioisotope, a fluorescent agent or a chromogenic agent. Radioisotopes can include without limitation; 3H, 32P, 33P and 35S etc. Fluorescent agents can include without limitation: FITC, texas red, rhodamine, etc.

The probe used in detection that is capable of hybridizing to MTAP nucleic acid can be from about 8 nucleotides to about 100 nucleotides, from about 10 nucleotides to about 75 nucleotides, from about 15 nucleotides to about 50 nucleotides, or about 20 to about 30 nucleotides. The kit can also provide instructions for analysis of patient cancer samples, wherein the presence or absence of MTAP deficiency indicates if the subject is sensitive or insensitive to treatment with a PRMT5 inhibitor.

Single stranded conformational polymorphism (SSCP) can also be used to detect MTAP deficiency. This technique is well described in Orita et al., PNAS 1989, 86:2766-2770.

### Measurement of Gene Expression

Evaluation of MTAP deficiency and measurement of MTAP gene expression, and measurement of PRMT5 gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In one embodiment, gene expression is detected and quantitated by hybridization to a probe that specifically hybridizes to the appropriate probe for that biomarker. The probes also can be attached to a solid support for use in high throughput screening assays using methods known in the art.

In one embodiment, the expression level of a gene is determined through exposure of a nucleic acid sample to the probe-modified chip. Extracted nucleic acid is labeled, for example, with a fluorescent tag, preferably during an amplification step.

Hybridization of the labeled sample is performed at an appropriate stringency level. The degree of probe-nucleic acid hybridization is quantitatively measured using a detection device.

Alternatively, any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In one embodiment, the hybridized nucleic acids are detected by detecting one or more labels attached to the sample nucleic acids. The labels can be incorporated by any of a number of means well known to those of skill in the art. However, in one embodiment, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, poly A, mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

In one example, the gene expression can be measured through an in-situ hybridization protocol that can detect RNA molecules on a slide containing tissue sections or cells (e.g., through RNAscope®).

Detectable labels suitable for use in the methods disclosed herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

### Detection of Polypeptides

Protein levels of MTAP can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

### Adjacent Biomarkers

Near or adjacent to MTAP on chromosome 9 are several other biomarkers. CDKN2A is often, if not usually, deleted along with MTAP. Additional genes or pseudogenes in this region include: C9orf53, ERVFRD-3, TUBB8P1, KHSRPP1, MIR31, and MIR31HG.

In some embodiments of the methods, the cell that is MTAP-deficient is also deficient in CDKN2A. In some embodiments, the cell that is MTAP-deficient is also deficient in one or more of: CDKN2A, C9orf53, ERVFRD-3, TUBB8P1, KHSRPP1, MIR31, and MIR31HG.

Thus, in various methods involving a step of evaluating a cell for MTAP deficiency or determining if a cell is MTAP-deficient, this step can comprise the step of determining if the cell is deficient for one or more of these markers: CDKN2A, C9orf53, ERVFRD-3, TUBB8P1, KHSRPP1, MIR31, and MIR31HG.

Thus, in some embodiments, the disclosure encompasses: A method of determining if a subject having or having been diagnosed with a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from said subject for MTAP deficiency, and evaluating a reference sample from a non-cancerous or normal control subject for MTAP deficiency, wherein MTAP deficiency in the test sample relative to the reference sample indicates that the subject will respond to therapeutic treatment with a PRMT5 inhibitor (e.g., an MTA-uncompetitive PRMT5 inhibitor, e.g., a compound of Formula (A) or a compound of Table 1, or pharmaceutically acceptable salts thereof); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

In some embodiments, the disclosure encompasses: A method of determining if a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from a subject having or having been diagnosed with said cancer for MTAP deficiency, and evaluating a reference sample from a non-cancerous or normal control subject for MTAP deficiency, wherein MTAP deficiency in the test sample relative to the reference sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive PRMT5 inhibitor, e.g., a compound of Formula (A) or a compound of Table 1, or pharmaceutically acceptable salts thereof) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

### Assaying for Biomarkers and PRMT5 Inhibitor Treatment

A number of patient stratification strategies could be employed to find patients likely to be sensitive to PRMT5 inhibition with an MTA-uncompetitive, non-competitive, or mixed mode PRMT5 inhibitor or an MTA-cooperative binding agent (e.g., a PRMT5 inhibitor of the present disclosure, e.g., a compound of Formula (A) or a compound of Table 1, or pharmaceutically acceptable salts thereof), including but not limited to, testing for MTAP deficiency and/or MTA accumulation.

Once a patient has been assayed for MTAP deficiency and/or MTA accumulation and predicted to be sensitive to treatment with a PRMT5 inhibitor, administration of any PRMT5 inhibitor (e.g., an MTA-uncompetitive, non-competitive, or mixed mode PRMT5 inhibitor or an MTA-cooperative binding agent, e.g., a compound of Formula (A) or a compound of Table 1, or pharmaceutically acceptable salts thereof) to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

### Kits

In some embodiments provided are kits related to methods of use described herein.

In one embodiment, provided is a kit for predicting the sensitivity of a subject having or having been diagnosed with an MTAP-deficiency-related cancer for treatment with a PRMT5 inhibitor is provided. The kit comprises: i) reagents capable of detecting human MTAP-deficient and/or MTA-accumulating cancer cells; and ii) instructions for how to use said kit.

### Selected Embodiments

Embodiment 1. A compound of Formula (A) or a pharmaceutically acceptable salt thereof;
wherein:

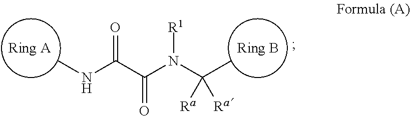

Ring A is selected from the group consisting of:

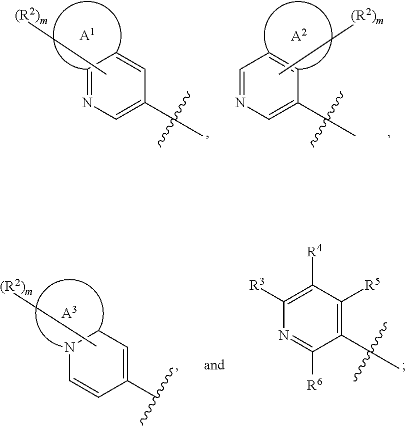

Ring B is selected from the group consisting of C₆-C₁₀ aryl and 5-10 membered heteroaryl, each optionally substituted at any available position;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₁-C₆ alkyl, —C₂-C₆ heteroalkyl, —C₂-C₆ haloalkyl, —C₃-C₁₀ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position;
each R² is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{af}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a} and R^{a}′ are independently selected from H and C₁-C₆ alkyl;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each Ro is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R²)₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu) and C₃-C₉ cycloalkyl (e.g., cyclopropyl, cyclobutyl, cyclopentyl, cyclohexyl); and
m is 0, 1, 2 or 3; wherein
(i) when R⁴ is —CH₃ then R³ is not H and;
(ii) the compound is not one of compounds a) to k) or a pharmaceutically acceptable salt thereof:
a)N1-benzyl-N2-(8-fluoroquinolin-3-yl)-N1-(2-(2-methoxyethoxy)ethyl)oxalamide:

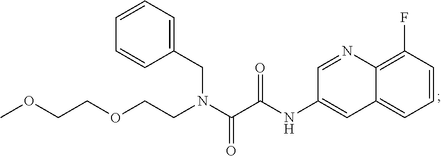 [SMILES: COCCOCCN(Cc1ccccc1)C(=O)C(=O)Nc1cnc2c(F)cccc2c1]

b)N1-(8-fluoroquinolin-3-yl)-N2-phenethyl-N2-(pyridin-4-ylmethyl)oxalamide:

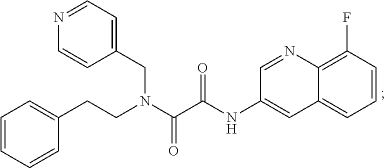 [SMILES: O=C(Nc1cnc2c(F)cccc2c1)C(=O)N(CCc1ccccc1)Cc1ccncc1]

c)N1-(6-amino-5,6,7,8-tetrahydroquinolin-3-yl)-N2-(3-fluoro-4-(pyridin-3-yl)benzyl)-N2-methyloxalamide:

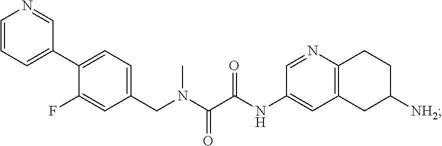 [SMILES: CN(Cc1ccc(-c2cccnc2)c(F)c1)C(=O)C(=O)Nc1cnc2c(c1)CC(N)CC2]

d)N1-cyclopentyl-N1-(3-fluorobenzyl)-N2-(8-fluoroquinolin-3-yl)oxalamide:

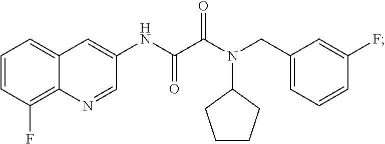 [SMILES: O=C(Nc1cnc2c(F)cccc2c1)C(=O)N(Cc1cccc(F)c1)C1CCCC1]

e)N1-(4-carbamoylbenzyl)-N2-(8-fluoroquinolin-3-yl)-N1-methyloxalamide:

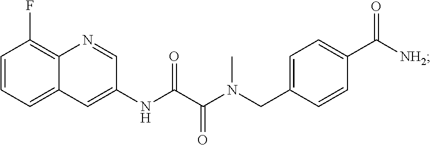 [SMILES: CN(Cc1ccc(C(N)=O)cc1)C(=O)C(=O)Nc1cnc2c(F)cccc2c1]

f)N1-benzyl-N1-methyl-N2-(quinolin-3-yl)oxalamide:

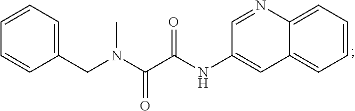 [SMILES: CN(Cc1ccccc1)C(=O)C(=O)Nc1cnc2ccccc2c1]

g)N1-([1,2,4]triazolo[4,3-a]pyridin-3-ylmethyl)-N2-(5-((dimethylamino)methyl)pyridin-3-yl)-N1-methyloxalamide:

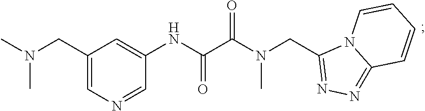 [SMILES: CN(C)Cc1cncc(NC(=O)C(=O)N(C)Cc2nnc3ccccn23)c1 ; ";"]

h)N1-(4-bromo-2-(3-chlorophenoxy)benzyl)-N2-(5-bromo-2-(4-methylpiperazin-1-yl)pyridin-3-yl)-N1-methyloxalamide:

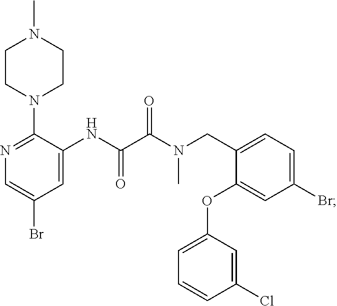 [SMILES: CN1CCN(c2ncc(Br)cc2NC(=O)C(=O)N(C)Cc2ccc(Br)cc2Oc2cccc(Cl)c2)CC1]

i) methyl 4-((N-methyl-2-oxo-2-((5-(trifluoromethyl)pyridin-3-yl)amino)acetamido)methyl)benzoate:

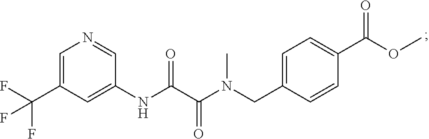 [SMILES: COC(=O)c1ccc(CN(C)C(=O)C(=O)Nc2cncc(C(F)(F)F)c2)cc1]

j)N1-methyl-N1-(2-methylbenzyl)-N2-(2-oxo-5-(trifluoromethyl)-1,2-dihydropyridin-3-yl)oxalamide:

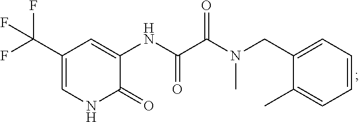 [SMILES: Cc1ccccc1CN(C)C(=O)C(=O)Nc1cc(C(F)(F)F)c[nH]c1=O ; ";"]

k)N1-(furan-2-ylmethyl)-N1-methyl-N2-(2-oxo-5-(trifluoromethyl)-1,2-dihydropyridin-3-yl)oxalamide:

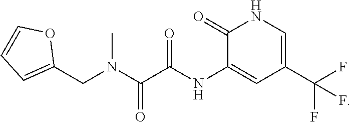 [SMILES: CN(Cc1ccco1)C(=O)C(=O)Nc1cc(C(F)(F)F)c[nH]c1=O]

Embodiment 2. A compound of Formula (A) or a pharmaceutically acceptable salt thereof;
wherein:

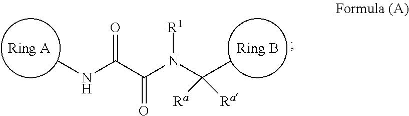

Ring A is selected from the group consisting of:

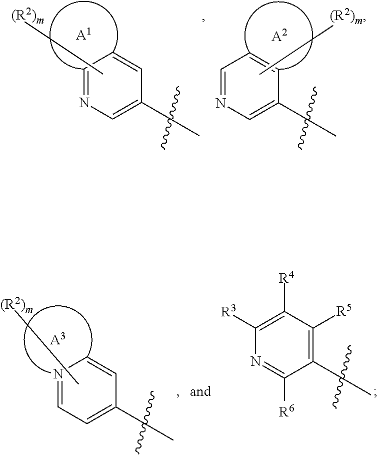

Ring B is selected from the group consisting of C₆-C₁₀ aryl and 5-10 membered heteroaryl, each optionally substituted at any available position;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₂-C₆ alkyl, —C₂-C₆ heteroalkyl, —C₂-C₆ haloalkyl, —C₃-C₉ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position;
each R² is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a} and R^{a}′ are independently selected from H and C₁-C₆ alkyl;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu) and C₃-C₉ cycloalkyl (e.g., cyclopropyl, cyclobutyl, cyclopentyl, cyclohexyl); and
m is 0, 1, 2 or 3; wherein
when R⁴ is —CH₃ then R³ is not H and;
the compound is not:
   a)N1-benzyl-N2-(8-fluoroquinolin-3-yl)-N1-(2-(2-methoxyethoxy)ethyl)oxalamide;
   b)N1-(8-fluoroquinolin-3-yl)-N2-phenethyl-N2-(pyridin-4-ylmethyl)oxalamide.

Embodiment 3. The compound of embodiment 1, wherein R^{a} and R^{a}′ are independently H and Me.

Embodiment 4. The compound of embodiment 1 or 2, wherein R^{a} is H.

Embodiment 5. The compound of embodiment 1 or 2, wherein R^{a} is Me.

Embodiment 6. The compound of any one of embodiments 1 to 5, wherein R^{a}′ is H.

Embodiment 7. The compound of any one of embodiments 1 to 5, wherein R^{a}′ is Me.

Embodiment 8. A compound of Formula (I) or a pharmaceutically acceptable salt thereof;
wherein:

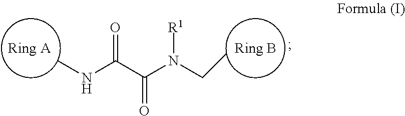

Ring A is selected from the group consisting of:

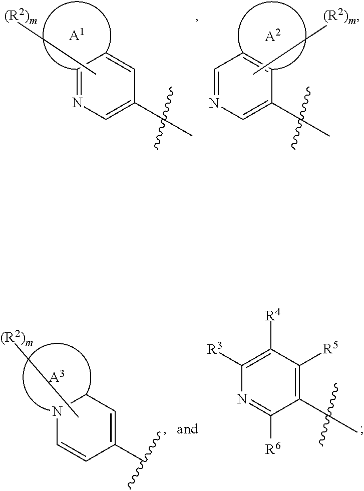

Ring B is selected from the group consisting of C₆-C₁₀ aryl and 5-10 membered heteroaryl, each optionally substituted at any available position;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₁-C₆ alkyl, —C₂-C₆ heteroalkyl, —C₂-C₆ haloalkyl, —C₃-C₁₀ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position;
each R² is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, -NRDC(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu) and C₃-C₉ cycloalkyl (e.g., cyclopropyl, cyclobutyl, cyclopentyl, cyclohexyl); and
m is 0, 1, 2 or 3; wherein
(i) when R⁴ is —CH₃ then R³ is not H and;
(ii) the compound is not one of compounds a) to k) or a pharmaceutically acceptable salt thereof:
a)N1-benzyl-N2-(8-fluoroquinolin-3-yl)-N1-(2-(2-methoxyethoxy)ethyl)oxalamide:

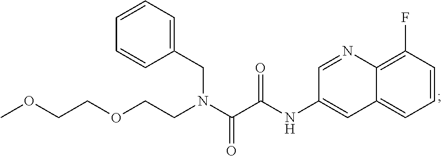 [SMILES: COCCOCCN(Cc1ccccc1)C(=O)C(=O)Nc1cnc2c(F)cccc2c1]

b)N1-(8-fluoroquinolin-3-yl)-N2-phenethyl-N2-(pyridin-4-ylmethyl)oxalamide:

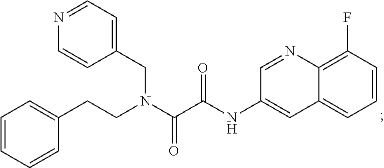 [SMILES: O=C(Nc1cnc2c(F)cccc2c1)C(=O)N(CCc1ccccc1)Cc1ccncc1]

c)N1-(6-amino-5,6,7,8-tetrahydroquinolin-3-yl)-N2-(3-fluoro-4-(pyridin-3-yl)benzyl)-N2-methyloxalamide:

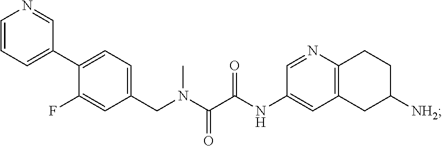 [SMILES: CN(Cc1ccc(-c2cccnc2)c(F)c1)C(=O)C(=O)Nc1cnc2c(c1)CC(N)CC2]

d)N1-cyclopentyl-N1-(3-fluorobenzyl)-N2-(8-fluoroquinolin-3-yl)oxalamide:

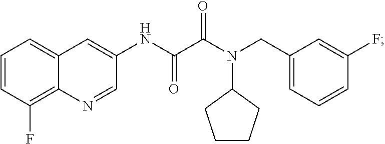 [SMILES: O=C(Nc1cnc2c(F)cccc2c1)C(=O)N(Cc1cccc(F)c1)C1CCCC1]

e)N1-(4-carbamoylbenzyl)-N2-(8-fluoroquinolin-3-yl)-N1-methyloxalamide:

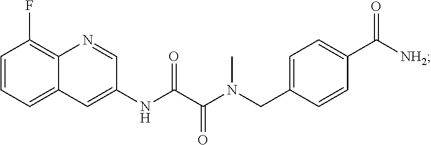 [SMILES: CN(Cc1ccc(C(N)=O)cc1)C(=O)C(=O)Nc1cnc2c(F)cccc2c1]

f)N1-benzyl-N1-methyl-N2-(quinolin-3-yl)oxalamide:

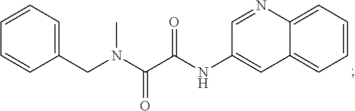 [SMILES: CN(Cc1ccccc1)C(=O)C(=O)Nc1cnc2ccccc2c1]

g)N1-([1,2,4]triazolo[4,3-a]pyridin-3-ylmethyl)-N2-(5-((dimethylamino)methyl)pyridin-3-yl)-N1-methyloxalamide:

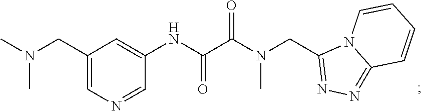 [SMILES: CN(C)Cc1cncc(NC(=O)C(=O)N(C)Cc2nnc3ccccn23)c1 ; ";"]

h)N1-(4-bromo-2-(3-chlorophenoxy)benzyl)-N2-(5-bromo-2-(4-methylpiperazin-1-yl)pyridin-3-yl)-N1-methyloxalamide;

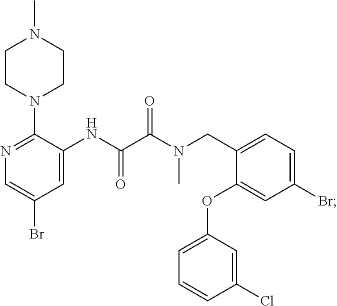 [SMILES: CN1CCN(c2ncc(Br)cc2NC(=O)C(=O)N(C)Cc2ccc(Br)cc2Oc2cccc(Cl)c2)CC1]

i) methyl 4-((N-methyl-2-oxo-2-((5-(trifluoromethyl)pyridin-3-yl)amino)acetamido)methyl)benzoate:

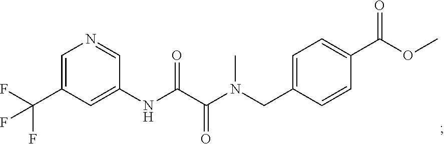 [SMILES: COC(=O)c1ccc(CN(C)C(=O)C(=O)Nc2cncc(C(F)(F)F)c2)cc1]

j)N1-methyl-N1-(2-methylbenzyl)-N2-(2-oxo-5-(trifluoromethyl)-1,2-dihydropyridin-3-yl)oxalamide:

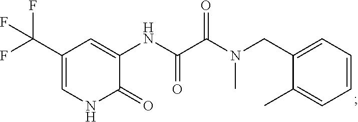 [SMILES: Cc1ccccc1CN(C)C(=O)C(=O)Nc1cc(C(F)(F)F)c[nH]c1=O ; ";"]

k)N1-(furan-2-ylmethyl)-N1-methyl-N2-(2-oxo-5-(trifluoromethyl)-1,2-dihydropyridin-3-yl)oxalamide:

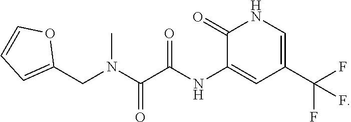 [SMILES: CN(Cc1ccco1)C(=O)C(=O)Nc1cc(C(F)(F)F)c[nH]c1=O]

Embodiment 9. A compound of Formula (I) or a pharmaceutically acceptable salt thereof;
wherein:

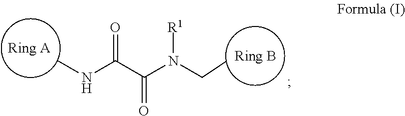

Ring A is selected from the group consisting of:

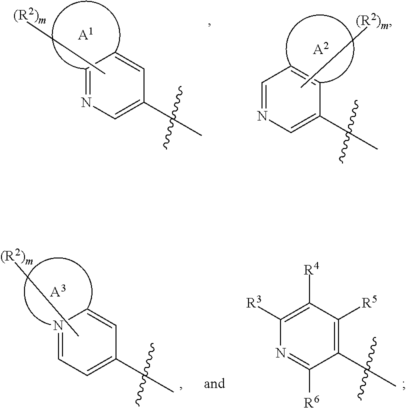

Ring B is selected from the group consisting of C₆-C₁₀ aryl and 5-10 membered heteroaryl, each optionally substituted at any available position;
each of rings A¹ and A² is independently 5-6 membered carbocyclyl, 5-6 membered heterocyclyl, 5-6 membered heteroaryl or phenyl;
each ring A³ is independently a 5-6 membered heterocyclyl or 5-6 membered heteroaryl, wherein the heterocyclyl and heteroaryl contain at least one nitrogen atom;
each R¹ is independently selected from the group consisting of —C₂-C₆ alkyl, —C₂-C₆ heteroalkyl, —C₂-C₆ haloalkyl, —C₃-C₉ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each optionally substituted at any available position;
each R² is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R², —SR^{a2}, —S(═O)(═NR²)R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a}C(═O)R^{a3}, —NR^{a}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, —Pr, -ⁿBu, -^{t}Bu, -sec-Bu,-iso-Bu) and C₃-C₉ cycloalkyl (e.g., cyclopropyl, cyclobutyl, cyclopentyl, cyclohexyl); and
m is 0, 1, 2 or 3; wherein
when R⁴ is —CH₃ then R³ is not H and;
the compound is not:
a)N1-benzyl-N2-(8-fluoroquinolin-3-yl)-N1-(2-(2-methoxyethoxy)ethyl)oxalamide;
b)N1-(8-fluoroquinolin-3-yl)-N2-phenethyl-N2-(pyridin-4-ylmethyl)oxalamide.

Embodiment 10. The compound of embodiment 1 or 2, wherein the compound is of Formula (I):

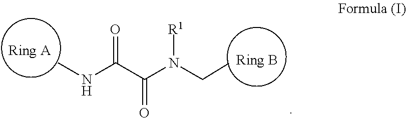

Embodiment 11. The compound of any one of embodiments 1 to 10, or a pharmaceutically acceptable salt thereof, wherein Ring A is selected from the group consisting of:

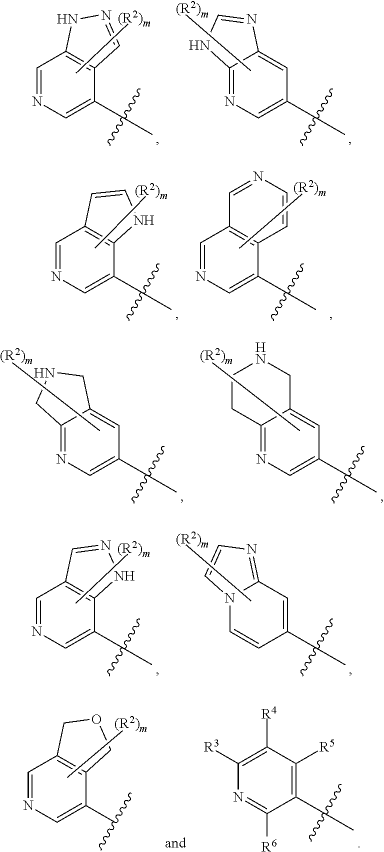

Embodiment 12. The compound of any one of embodiments 1 to 10, or a pharmaceutically acceptable salt thereof, wherein Ring A is selected from the group consisting of:

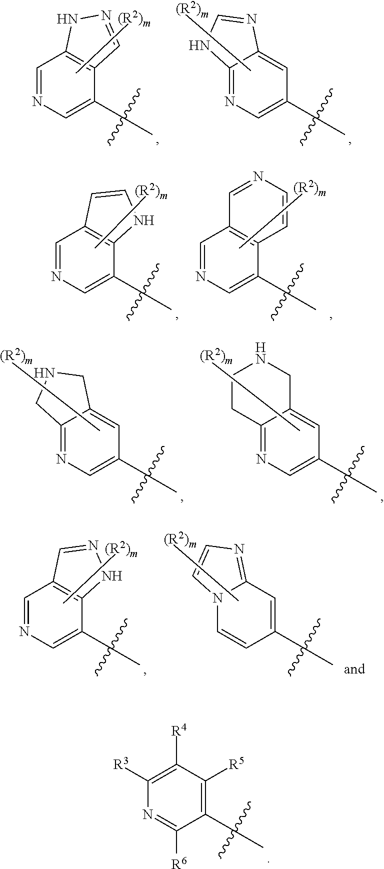

Embodiment 13. The compound of any one of embodiments 1 to 10, or a pharmaceutically acceptable salt thereof, wherein Ring A is selected from the group consisting of:

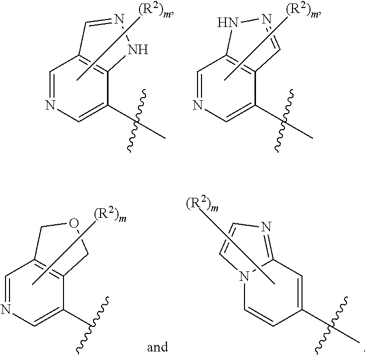

Embodiment 14. The compound of any one of embodiments 1 to 10, or a pharmaceutically acceptable salt thereof, wherein Ring A is selected from the group consisting of:

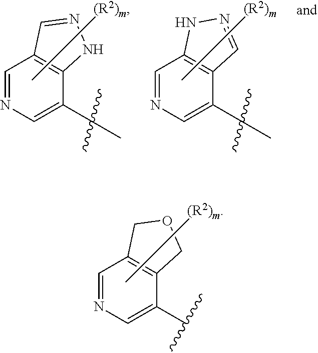

Embodiment 15. The compound of any one of embodiments 1 to 10, or a pharmaceutically acceptable salt thereof, wherein Ring A is selected from the group consisting of:

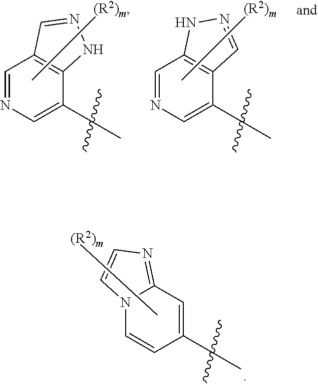

Embodiment 16. The compound of any one of embodiments 1 to 10, or a pharmaceutically acceptable salt thereof, wherein Ring A is selected from the group consisting of:

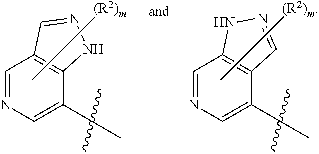

Embodiment 17. The compound of any one of embodiments 1 to 10, or a pharmaceutically acceptable salt thereof, wherein Ring A is selected from the group consisting of:

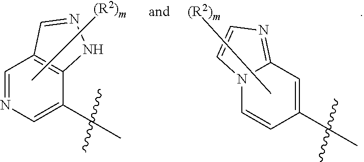

Embodiment 18. The compound of any one of embodiments 1 to 10, or a pharmaceutically acceptable salt thereof, wherein Ring A is

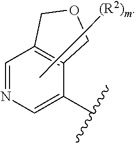

Embodiment 19. The compound of any one of embodiments 1 to 10, or a pharmaceutically acceptable salt thereof, wherein Ring A is

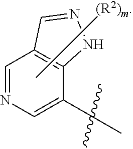

Embodiment 20. The compound of any one of embodiments 1 to 10, or a pharmaceutically acceptable salt thereof, wherein Ring A is

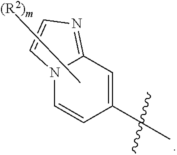

Embodiment 21. The compound of any one of embodiments 1 to 10, or a pharmaceutically acceptable salt thereof, wherein Ring A is

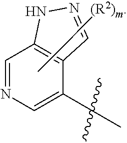

Embodiment 22. The compound of any one of embodiments 1 to 10, or a pharmaceutically acceptable salt thereof, wherein the compound is of Formula (A_1):

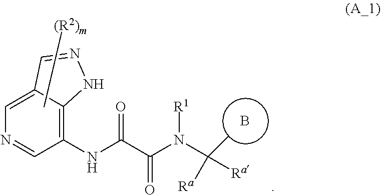

Embodiment 23. The compound of any one of embodiments 8 to 10, or a pharmaceutically acceptable salt thereof, wherein the compound is of Formula (I_1):

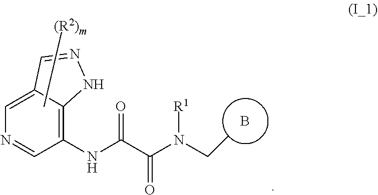

Embodiment 24. The compound of any one of embodiments 1 to 10, or a pharmaceutically acceptable salt thereof, wherein the compound is of Formula (A_3):

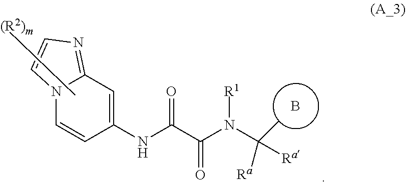

Embodiment 25. The compound of any one of embodiments 8 to 10, or a pharmaceutically acceptable salt thereof, wherein the compound is of Formula (I_3):

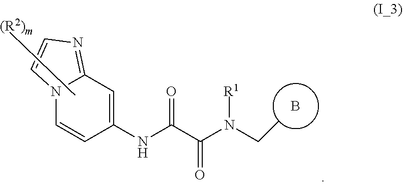

Embodiment 26. The compound of any one of embodiments 1 to 10, or a pharmaceutically acceptable salt thereof, wherein the compound is of Formula (A_4):

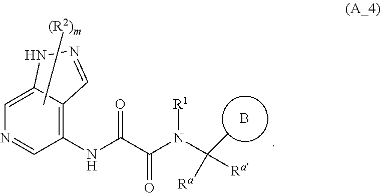

Embodiment 27. The compound of any one of embodiments 8 to 10, or a pharmaceutically acceptable salt thereof, wherein the compound is of Formula (I_4):

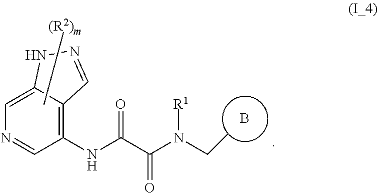

Embodiment 28. The compound of any one of embodiments 1 to 10, or a pharmaceutically acceptable salt thereof, wherein the compound is of Formula (A_5):

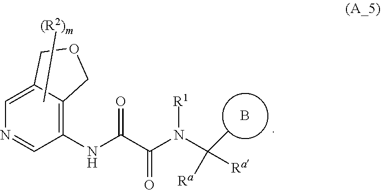

Embodiment 29. The compound of any one of embodiments 8 to 10, or a pharmaceutically acceptable salt thereof, wherein the compound is of Formula (1 5):

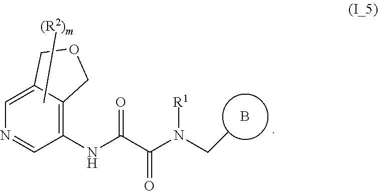

Embodiment 30. The compound of any one of embodiments 1 to 29, or a pharmaceutically acceptable salt thereof, wherein m is 0, 1 or 2.

Embodiment 31. The compound of any one of embodiments 1 to 29, or a pharmaceutically acceptable salt thereof, wherein m is 0 or 1.

Embodiment 32. The compound of any one of embodiments 1 to 29, or a pharmaceutically acceptable salt thereof, wherein m is 0.

Embodiment 33. The compound of any one of embodiments 1 to 29, or a pharmaceutically acceptable salt thereof, wherein m is 1.

Embodiment 34. The compound of any one of embodiments 1 to 29, or a pharmaceutically acceptable salt thereof, wherein m is 2.

Embodiment 35. The compound of any one of embodiments 1 to 29, or a pharmaceutically acceptable salt thereof, wherein m is 3.

Embodiment 36. The compound of any one of embodiments 1 to 10, or a pharmaceutically acceptable salt thereof, wherein Ring A is selected from the group consisting of:

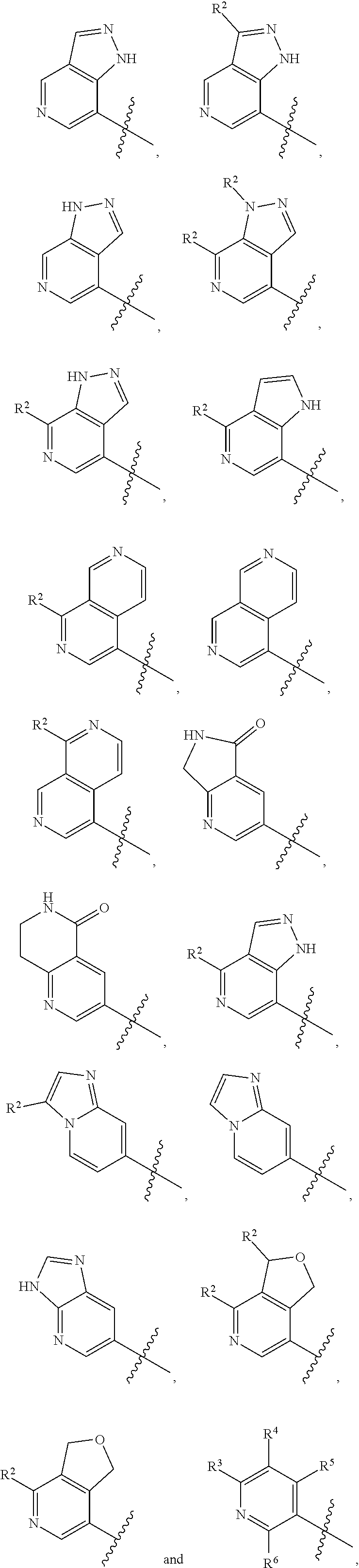

wherein
each R² is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, C(═O)OR^{a4}, —NR^{a4}C (═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of —O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, -NRDC(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu).and C₃-C₉ cycloalkyl (e.g., cyclopropyl, cyclobutyl, cyclopentyl, cyclohexyl).

Embodiment 37. The compound of any one of embodiments 1 to 10, or a pharmaceutically acceptable salt thereof, wherein Ring A is selected from the group consisting of:

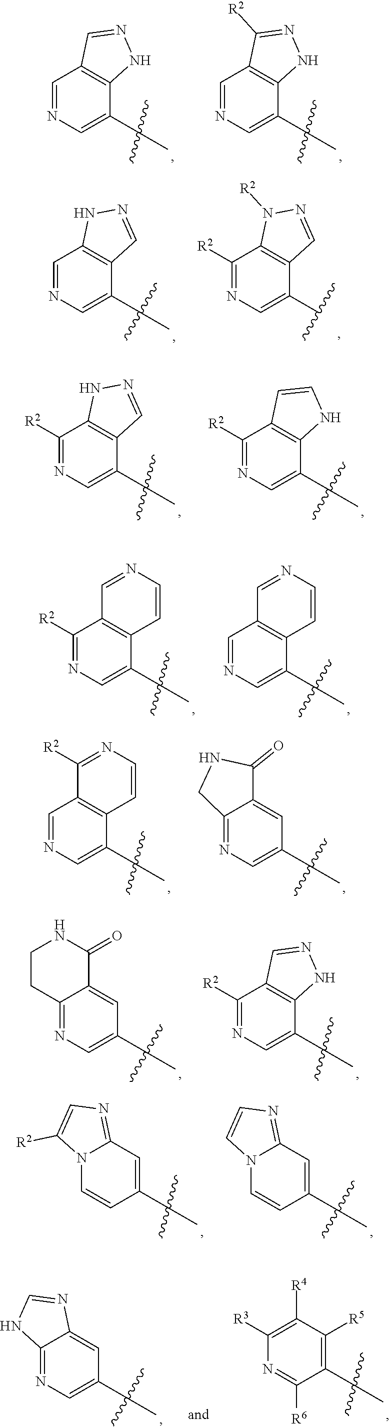

wherein
each R² is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR42, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)R^{a3}, —SR^{a3}, —S(═O)(═NR^{a3})R^{a3}, —NR^{a3}S(═O)₂R43 and —S(═O)₂N(R^{a3})₂;
each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, -—OR^{a4}, —N(R^{a4})₂, —C(═O)R^{a4}, —C(═O)OR^{a4}, —NR^{a4}C(═O)R⁴, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu). and C₃-C₉ cycloalkyl (e.g., cyclopropyl, cyclobutyl, cyclopentyl, cyclohexyl).

Embodiment 38. The compound of any one of embodiments 1 to 10, or a pharmaceutically acceptable salt thereof, wherein Ring A is selected from the group consisting of:

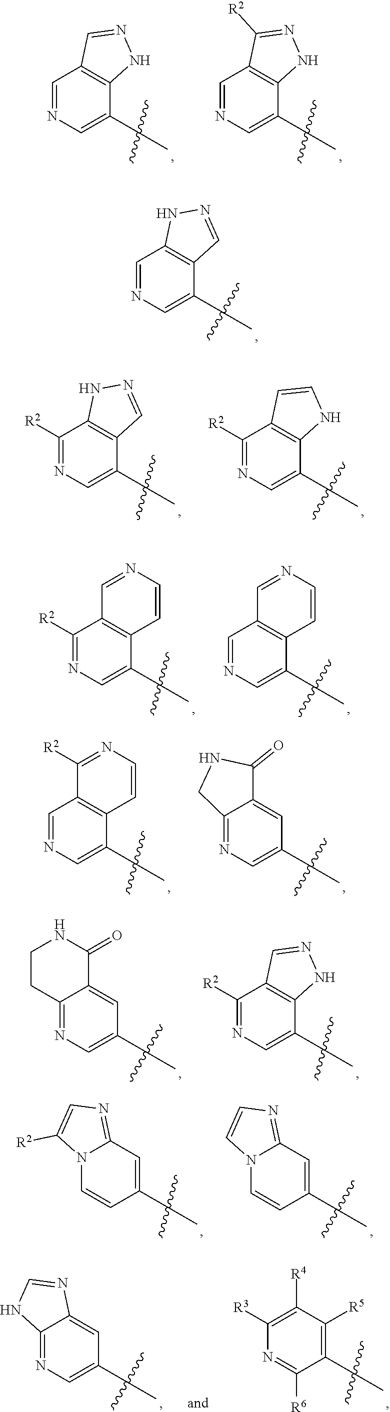

wherein
each R² is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C (═O)OR^{a2}, —C(═O)N(R^{a2})₂, —C(═O)N(OR^{a2})(R^{a2}), —OC(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂;
each R³ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a3}, —N(R^{a3})₂, —C(═O)R^{a3}, C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂;
each R⁴ is independently selected from the group consisting of D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O) ¿R^{a4}, —SR^{a4}, —S(═O)(═NR^{a4})R^{a4}, —NR^{a4}S(═O)>R^{a4} and —S(═O)₂N(R^{a4})₂;
each R⁵ is independently selected from the group consisting of H, D, halo, —CN, —C₁-C₆ alkyl, —C₁-C₆ heteroalkyl, —C₁-C₆ haloalkyl, -C3-Co cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C (═O)R^{a5}, —NR^{a5}C(═O)OR^{a5}, —C(O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂;
each R⁶ is independently selected from the group consisting of H, D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, —OC(═O)N(R^{a6})₂, —S(═O)R^{a6}, —S(═O)₂R^{a6}, —SR^{a6}, —S(═O)(═NR^{a6})R^{a6}, —NR^{a6}S(═O)₂R^{a6} and —S(═O)₂N(R^{a6})₂;
each R^{a2}, R^{a3}, R^{a4}, R^{a5} and R^{a6} is independently selected from the group consisting of H, —C₁-C₆ alkyl, —C₁-C₆ heteroalkyl, C₃-C₉ cycloalkyl, 3-7 membered heterocyclyl, cycloalkylalkyl, heterocyclylalkyl, aryl, 5-6 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁹, wherein each R⁹ is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, -NRDC(═O)R^{b}, -NRDC(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, -ⁱPr, -ⁿBu, -^{t}Bu, -sec-Bu,-iso-Bu).and C₃-C₉ cycloalkyl (e.g., cyclopropyl, cyclobutyl, cyclopentyl, cyclohexyl).

Embodiment 39. The compound of embodiment 36, or a pharmaceutically acceptable salt thereof, wherein Ring A is selected from the group consisting of:

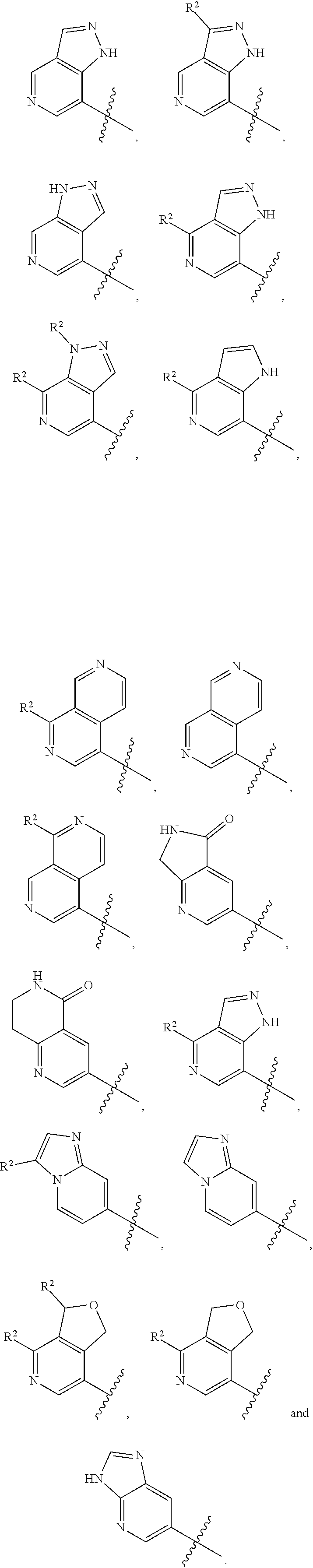

Embodiment 40. The compound of embodiment 36, or a pharmaceutically acceptable salt thereof, wherein Ring A is selected from the group consisting of:

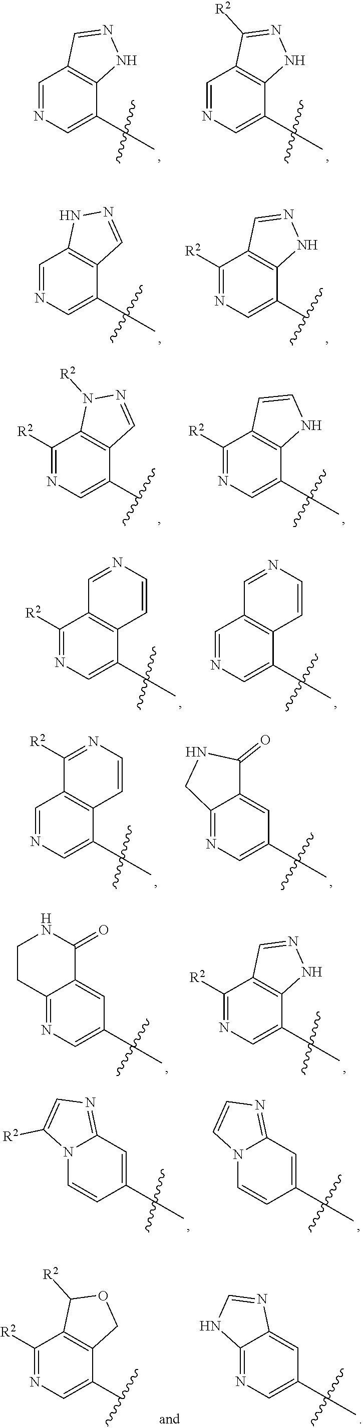

Embodiment 41. The compound of embodiment 36 or 37, or a pharmaceutically acceptable salt thereof, wherein Ring A is selected from the group consisting of:

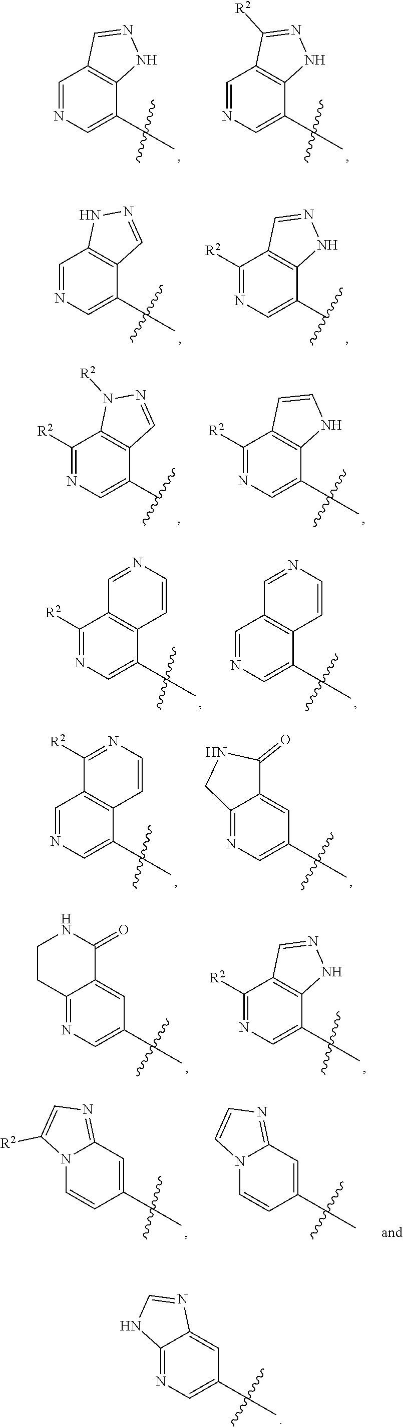

Embodiment 42. The compound of any one of embodiments 36-38, or a pharmaceutically acceptable salt thereof, wherein Ring A is selected from the group consisting of:

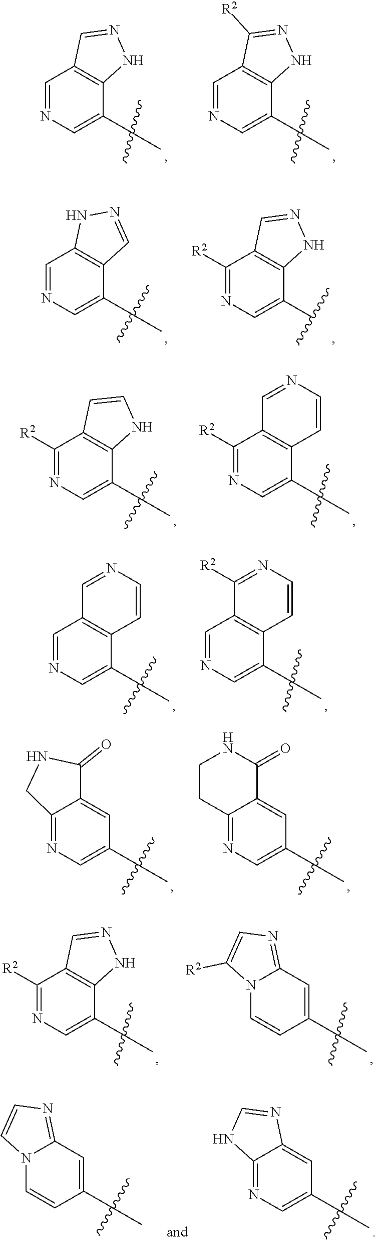

Embodiment 43. The compound of embodiment 36, or a pharmaceutically acceptable salt thereof, wherein Ring A is selected from the group consisting of:

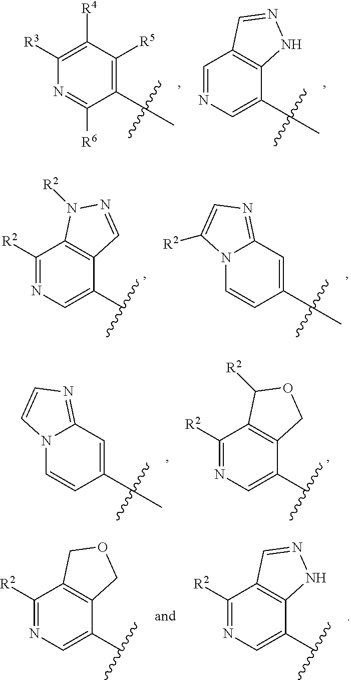

Embodiment 44. The compound of embodiment 36 or 37, or a pharmaceutically acceptable salt thereof, wherein Ring A is selected from the group consisting of:

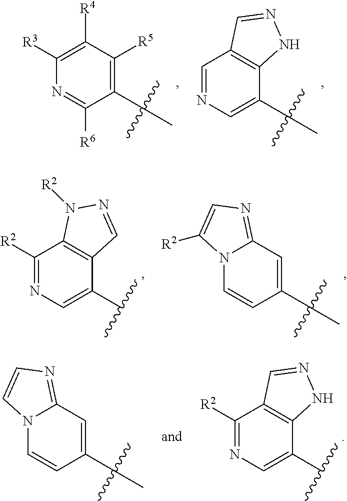

Embodiment 45. The compound of embodiment 36, or a pharmaceutically acceptable salt thereof, wherein Ring A is selected from the group consisting of:

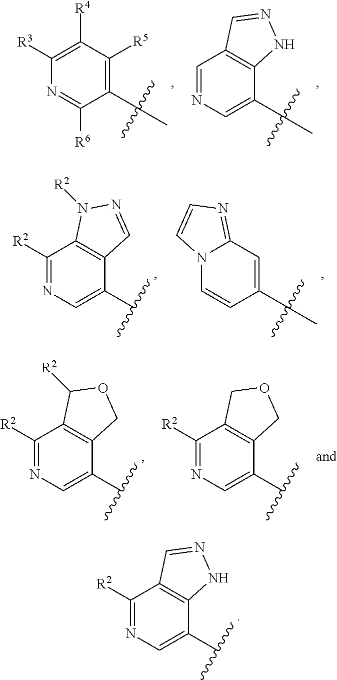

Embodiment 46. The compound of embodiment 36 or 37, or a pharmaceutically acceptable salt thereof, wherein Ring A is selected from the group consisting of:

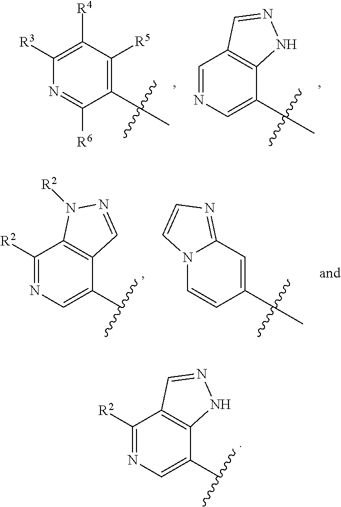

Embodiment 47. The compound of embodiment 37 or 38, or a pharmaceutically acceptable salt thereof, wherein Ring A is selected from the group consisting of:

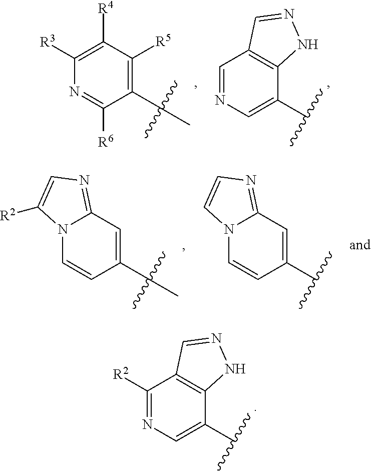

Embodiment 48. The compound of embodiment 36, or a pharmaceutically acceptable salt thereof, wherein Ring A is selected from the group consisting of:

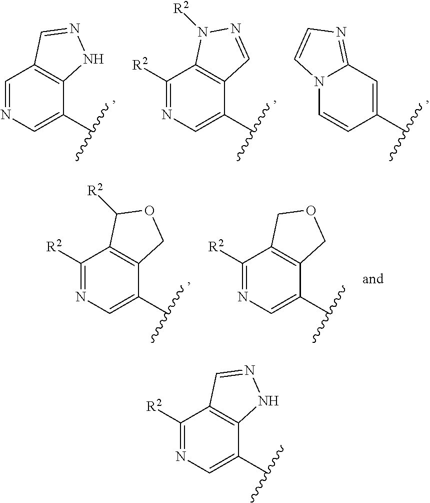

Embodiment 49. The compound of embodiment 36 or 37, or a pharmaceutically acceptable salt thereof, wherein Ring A is selected from the group consisting of:

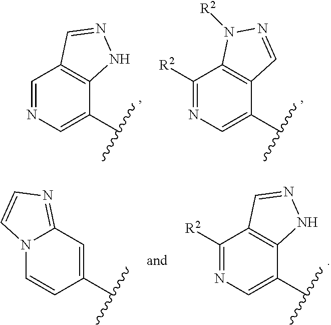

Embodiment 50. The compound of any one of embodiments 36-38, or a pharmaceutically acceptable salt thereof, wherein Ring A is selected from the group consisting of:

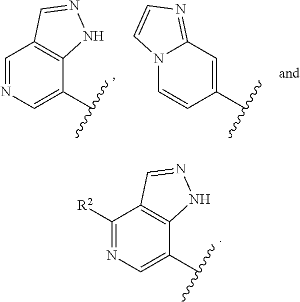

Embodiment 51. The compound of embodiment 36, or a pharmaceutically acceptable salt thereof, wherein Ring A is selected from the group consisting of:

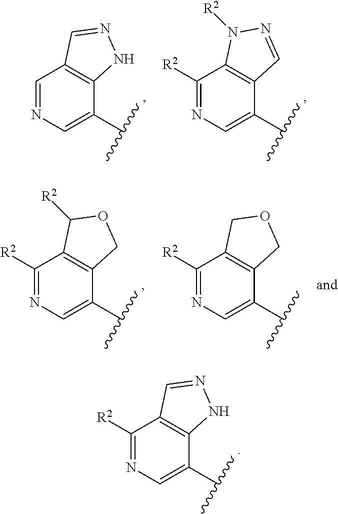

Embodiment 52. The compound of embodiment 36, or a pharmaceutically acceptable salt thereof, wherein Ring A is selected from the group consisting of:

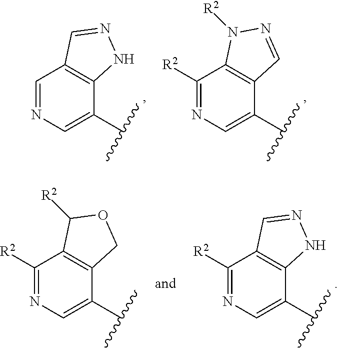

Embodiment 53. The compound of embodiment 36 or 37, or a pharmaceutically acceptable salt thereof, wherein Ring A is selected from the group consisting of:

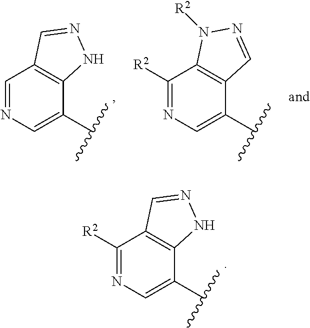

Embodiment 54. The compound of embodiment 36 or 37, or a pharmaceutically acceptable salt thereof, wherein Ring A is selected from the group consisting of:

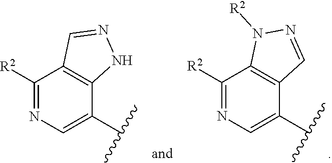

Embodiment 55. The compound of embodiment 36 or 37, or a pharmaceutically acceptable salt thereof, wherein Ring A is selected from the group consisting of:

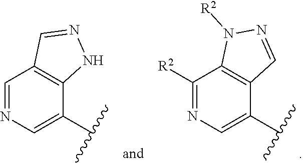

Embodiment 56. The compound of embodiment 36, or a pharmaceutically acceptable salt thereof, wherein Ring A is

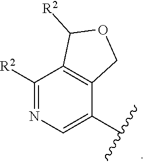

Embodiment 57. The compound of embodiment 36, or a pharmaceutically acceptable salt thereof, wherein Ring A is

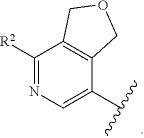

Embodiment 58. The compound of any one of embodiments 36-38, or a pharmaceutically acceptable salt thereof, wherein Ring A is

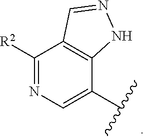

Embodiment 59. The compound of embodiment 36 or 37, or a pharmaceutically acceptable salt thereof, wherein Ring A is

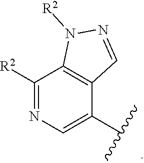

Embodiment 60. The compound of any one of embodiments 36-38, or a pharmaceutically acceptable salt thereof, wherein Ring A is

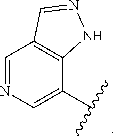 [SMILES: *c1cncc2cn[nH]c12]

Embodiment 61. The compound of embodiment 37 or 38, or a pharmaceutically acceptable salt thereof, wherein Ring A is

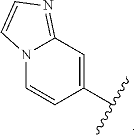 [SMILES: *c1ccn2ccnc2c1]

Embodiment 62. The compound of any one of embodiments 36-38, or a pharmaceutically acceptable salt thereof, wherein the compound is of Formula (A_1a):

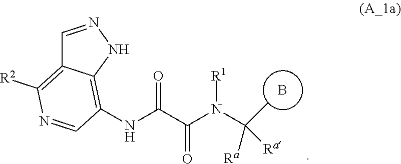

Embodiment 63. The compound of any one of embodiments 36-38, or a pharmaceutically acceptable salt thereof, wherein the compound is of Formula (I_1a):

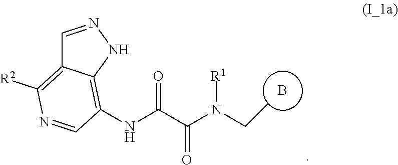

Embodiment 64. The compound of any one of embodiments 36-38, or a pharmaceutically acceptable salt thereof, wherein the compound is of Formula (A_1c):

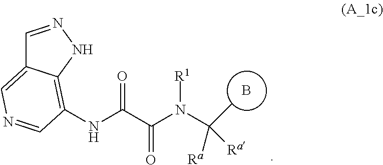

Embodiment 65. The compound of any one of embodiments 36-38, or a pharmaceutically acceptable salt thereof, wherein the compound is of Formula (I_1c):

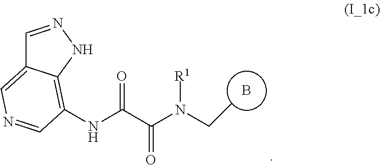

Embodiment 66. The compound of any one of embodiments 36-38, or a pharmaceutically acceptable salt thereof, wherein the compound is of Formula (A_3a):

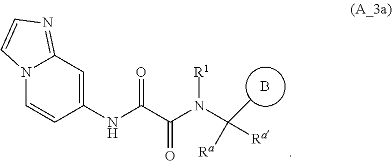

Embodiment 67. The compound of any one of embodiments 36-38, or a pharmaceutically acceptable salt thereof, wherein the compound is of Formula (I_3a):

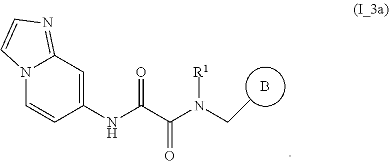

Embodiment 68. The compound of embodiment 36 or 37, or a pharmaceutically acceptable salt thereof, wherein the compound is of Formula (A_4a):

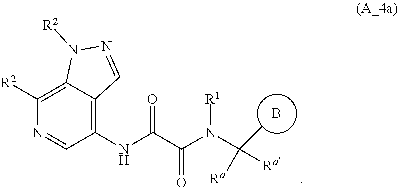

Embodiment 69. The compound of embodiment 36 or 37, or a pharmaceutically acceptable salt thereof, wherein the compound is of Formula (I_4a):

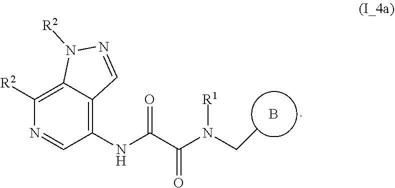

Embodiment 70. The compound of embodiment 36, or a pharmaceutically acceptable salt thereof, wherein the compound is of Formula (A_5a):

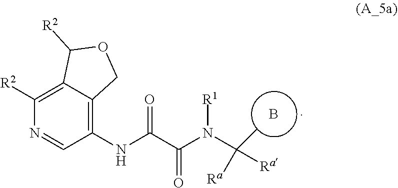

Embodiment 71. The compound of embodiment 36, or a pharmaceutically acceptable salt thereof, wherein the compound is of Formula (I_5a):

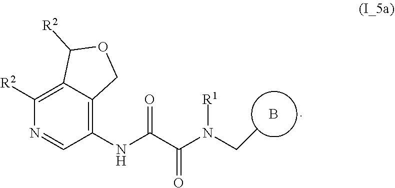

Embodiment 72. The compound of embodiment 36, or a pharmaceutically acceptable salt thereof, wherein the compound is of Formula (A_5b):

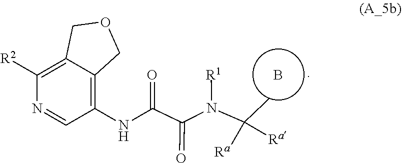

Embodiment 73. The compound of embodiment 36, or a pharmaceutically acceptable salt thereof, wherein the compound is of Formula (I_5b):

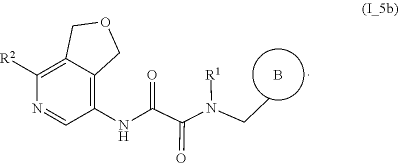

Embodiment 74. The compound of any one of embodiments 36-38, or a pharmaceutically acceptable salt thereof, wherein the compound is of Formula (A_2):

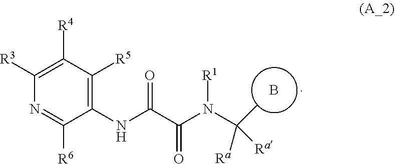

Embodiment 75. The compound of embodiment any one of embodiments 36-38, or a pharmaceutically acceptable salt thereof, wherein the compound is of Formula (I_2):

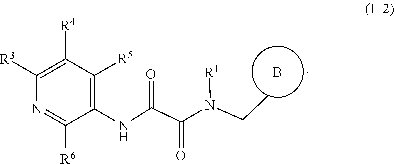

Embodiment 76. The compound of any one of embodiments 1 to 59, 62, 63 and 68-73, or a pharmaceutically acceptable salt thereof, wherein each R² is independently selected from the group consisting of -D, halo, ═O, —CN, —C₁-C₆ alkyl, —C₁-C₆ heteroalkyl, —C₁-C₆ haloalkyl, -C3-C, cycloalkyl, -OR$2, —N(R^{a2})₂, C(═O)R^{a2}, C(═O)OR^{a2}, —NR^{a2}C(═O)R^{a4}, —NR^{a2}C(═O)OR^{a2}, —C(═O)N(R^{a2})₂, —C(═O)N(OR^{a2})(R^{a2}) and —OC(═O)N(R^{a2})₂.

Embodiment 77. The compound of any one of embodiments 1 to 59, 62, 63, and 68-73, or a pharmaceutically acceptable salt thereof, wherein each R² is independently selected from the group consisting of -D, ═O, —C₁-C₆ alkyl, —C₁-C₆ haloalkyl, —C₃-C₉ cycloalkyl, —OR^{a2} and —N(R^{a2})₂.

Embodiment 78. The compound of any one of embodiments 1 to 59, 62, 63, and 68-73, or a pharmaceutically acceptable salt thereof, wherein each R² is independently selected from the group consisting of -D, ═O, —C₁-C₆ alkyl and —N(R^{a2})₂.

Embodiment 79. The compound of any one of embodiments 1 to 59, 62, 63, 68-73 and 76 to 78, or a pharmaceutically acceptable salt thereof, wherein each R^{a2} is independently selected from the group consisting of H and C₁-C₆ alkyl.

Embodiment 80. The compound of any one of embodiments 1 to 59, 62, 63, 68-73 and 76 to 78, or a pharmaceutically acceptable salt thereof, wherein each R^{a2} is H.

Embodiment 81. The compound of any one of embodiments 1 to 59, 62, 63 and 68-73, or a pharmaceutically acceptable salt thereof, wherein each R² is independently selected from the group consisting of -D, ═O, -Me, -Et, —Pr, -^{t}Bu, —NH₂, —NHCH₃ and —NH(CH₃)₂.

Embodiment 82. The compound of any one of embodiments 1 to 59, 62, 63 and 68-73, or a pharmaceutically acceptable salt thereof, wherein each R² is independently selected from —NH₂ and -Me.

Embodiment 83. The compound of any one of embodiments 1 to 59, 62, 63 and 68-73, or a pharmaceutically acceptable salt thereof, wherein each R² is —NH₂.

Embodiment 84. The compound of embodiment 36, or a pharmaceutically acceptable salt thereof, wherein Ring A is selected from the group consisting of:

Embodiment 85.

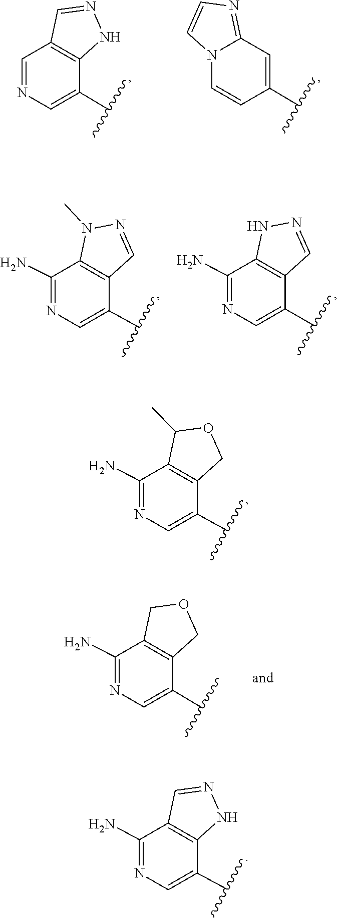

The compound of embodiment 36 or 37, or a pharmaceutically acceptable salt thereof, wherein Ring A is selected from the group consisting of:

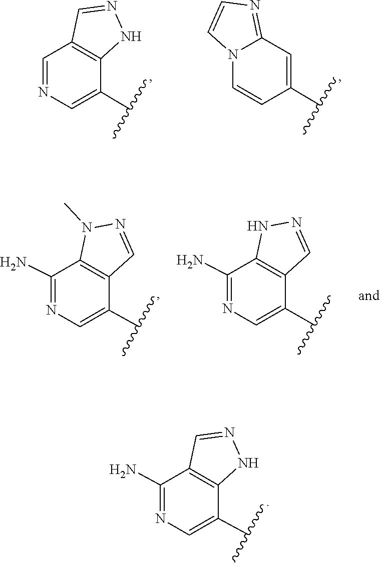 [SMILES: *c1cncc2cn[nH]c12 ; *c1ccn2ccnc2c1 ; *c1cnc(N)c2c1cnn2C ; *c1cnc(N)c2[nH]ncc12 ; *c1cnc(N)c2cn[nH]c12]

Embodiment 86. The compound of embodiment 36, or a pharmaceutically acceptable salt thereof, wherein Ring A is selected from the group consisting of:

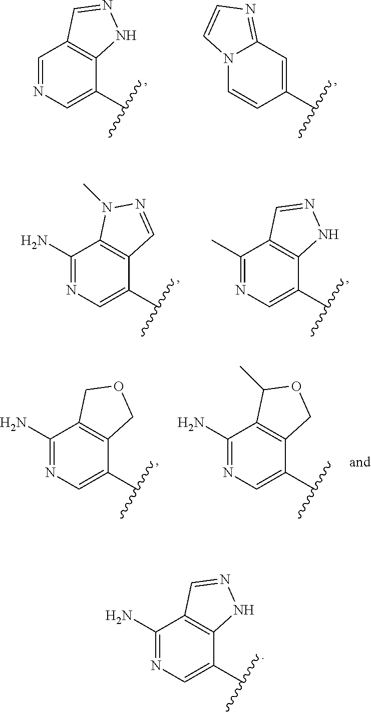

Embodiment 87. The compound of embodiment 36, or a pharmaceutically acceptable salt thereof, wherein Ring A is selected from the group consisting of:

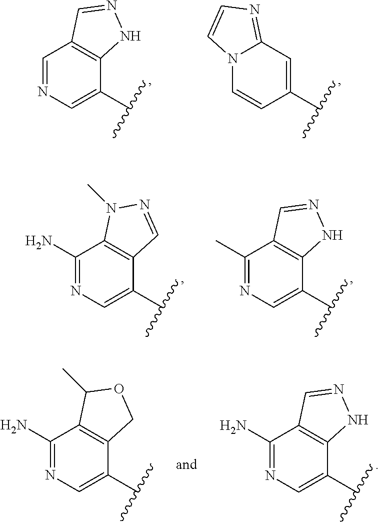 [SMILES: *c1cncc2cn[nH]c12 ; *c1ccn2ccnc2c1 ; *c1cnc(N)c2c1cnn2C ; *c1cnc(C)c2cn[nH]c12 ; *c1cnc(N)c2c1COC2C ; *c1cnc(N)c2cn[nH]c12]

Embodiment 88. The compound of embodiment 36 or 37, or a pharmaceutically acceptable salt thereof, wherein Ring A is selected from the group consisting of:

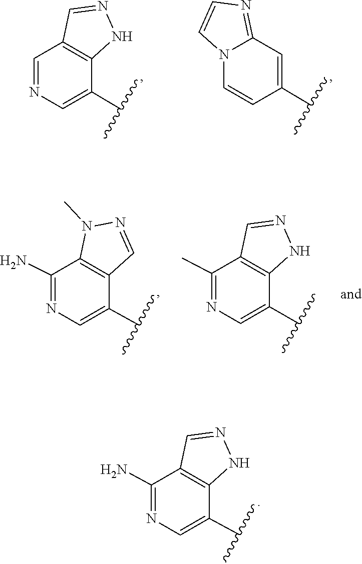 [SMILES: *c1cncc2cn[nH]c12 ; *c1ccn2ccnc2c1 ; *c1cnc(N)c2c1cnn2C ; *c1cnc(C)c2cn[nH]c12 ; *c1cnc(N)c2cn[nH]c12]

Embodiment 89. The compound of embodiment 36, or a pharmaceutically acceptable salt thereof, wherein Ring A is selected from the group consisting of:

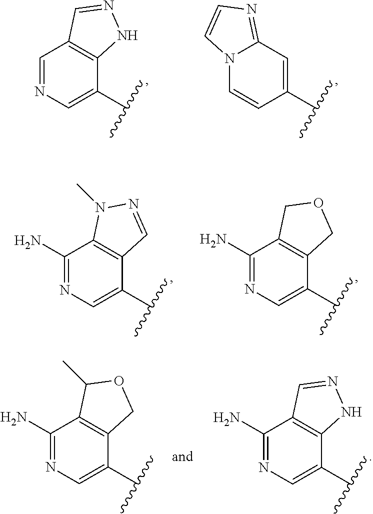 [SMILES: *c1cncc2cn[nH]c12 ; *c1ccn2ccnc2c1 ; *c1cnc(N)c2c1cnn2C ; *c1cnc(N)c2c1COC2 ; *c1cnc(N)c2c1COC2C ; *c1cnc(N)c2cn[nH]c12]

Embodiment 90. The compound of embodiment 36, or a pharmaceutically acceptable salt thereof, wherein Ring A is selected from the group consisting of:

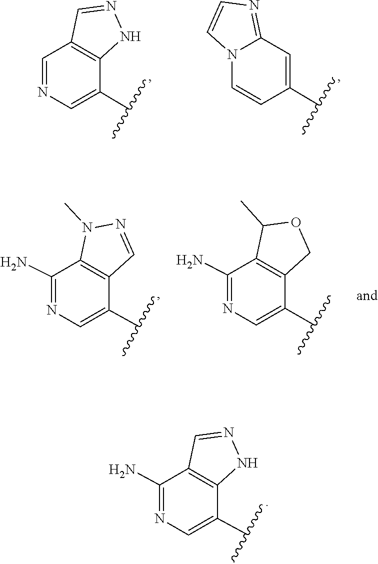 [SMILES: *c1cncc2cn[nH]c12 ; *c1ccn2ccnc2c1 ; *c1cnc(N)c2c1cnn2C ; *c1cnc(N)c2c1COC2C ; *c1cnc(N)c2cn[nH]c12]

Embodiment 91. The compound of embodiment 36 or 37, or a pharmaceutically acceptable salt thereof, wherein Ring A is selected from the group consisting of:

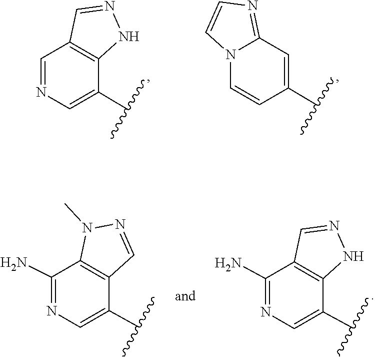 [SMILES: *c1cncc2cn[nH]c12 ; *c1ccn2ccnc2c1 ; *c1cnc(N)c2c1cnn2C ; *c1cnc(N)c2cn[nH]c12]

Embodiment 92. The compound of embodiment 36, or a pharmaceutically acceptable salt thereof, wherein Ring A is selected from the group consisting of:

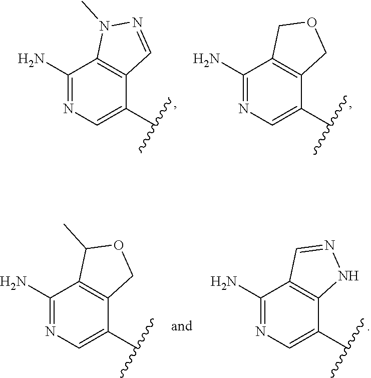 [SMILES: *c1cnc(N)c2c1cnn2C ; *c1cnc(N)c2c1COC2 ; *c1cnc(N)c2c1COC2C ; *c1cnc(N)c2cn[nH]c12]

Embodiment 93. The compound of embodiment 36, or a pharmaceutically acceptable salt thereof, wherein Ring A is selected from the group consisting of:

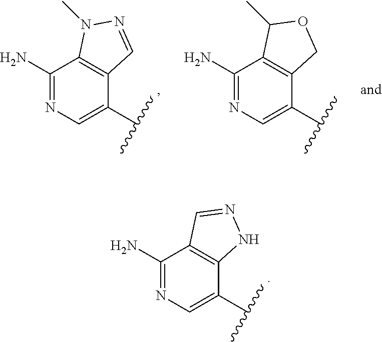 [SMILES: *c1cnc(N)c2c1cnn2C ; *c1cnc(N)c2c1COC2C ; *c1cnc(N)c2cn[nH]c12]

Embodiment 94. The compound of embodiment 36 or 37, or a pharmaceutically acceptable salt thereof, wherein Ring A is selected from the group consisting of:

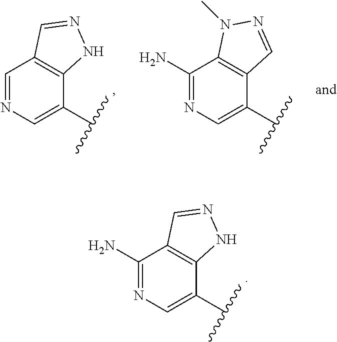 [SMILES: *c1cncc2cn[nH]c12 ; *c1cnc(N)c2c1cnn2C ; *c1cnc(N)c2cn[nH]c12]

Embodiment 95. The compound of any one of embodiments 36-38, or a pharmaceutically acceptable salt thereof, wherein Ring A is selected from the group consisting of:

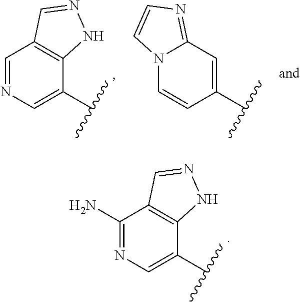

Embodiment 96. The compound of embodiment 36 or 37, or a pharmaceutically acceptable salt thereof, wherein Ring A is selected from the group consisting of:

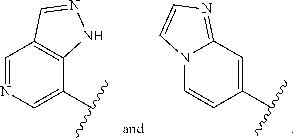 [SMILES: *c1cncc2cn[nH]c12 ; *c1ccn2ccnc2c1]

Embodiment 97. The compound of embodiment 36 or 37, or a pharmaceutically acceptable salt thereof, wherein Ring A is selected from the group consisting of:

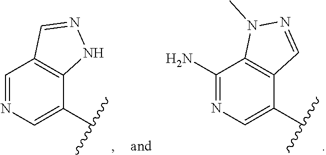 [SMILES: *c1cncc2cn[nH]c12 ; *c1cnc(N)c2c1cnn2C]

Embodiment 98. The compound of embodiment 36 or 37, or a pharmaceutically acceptable salt thereof, wherein Ring A is selected from the group consisting of:

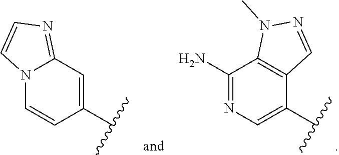 [SMILES: *c1ccn2ccnc2c1 ; *c1cnc(N)c2c1cnn2C]

Embodiment 99. The compound of any one of embodiments 36-38, or a pharmaceutically acceptable salt thereof, wherein Ring A is selected from the group consisting of:

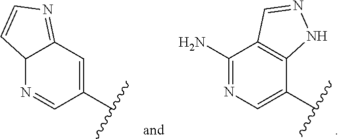 [SMILES: *C1=CC2=NC=CC2N=C1 ; *c1cnc(N)c2cn[nH]c12]

Embodiment 100. The compound of embodiment 36 or 37, or a pharmaceutically acceptable salt thereof, wherein Ring A is selected from the group consisting of:

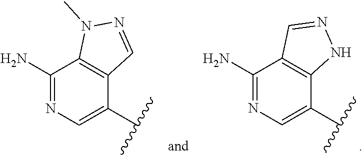 [SMILES: *c1cnc(N)c2c1cnn2C ; *c1cnc(N)c2cn[nH]c12]

Embodiment 101. The compound of any one of embodiments 36-38, or a pharmaceutically acceptable salt thereof, wherein Ring A is

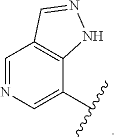 [SMILES: *c1cncc2cn[nH]c12]

Embodiment 102. The compound of any one of embodiments 36-38, or a pharmaceutically acceptable salt thereof, wherein Ring A is

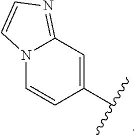 [SMILES: *c1ccn2ccnc2c1]

Embodiment 103. The compound of embodiment 36, or a pharmaceutically acceptable salt thereof, wherein Ring A is

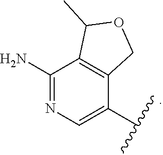 [SMILES: *c1cnc(N)c2c1COC2C]

Embodiment 104. The compound of embodiment 36, or a pharmaceutically acceptable salt thereof, wherein Ring A is

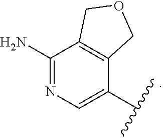 [SMILES: *c1cnc(N)c2c1COC2]

Embodiment 105. The compound of embodiment 37, or a pharmaceutically acceptable salt thereof, wherein Ring A is

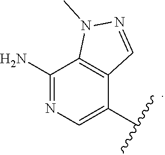 [SMILES: *c1cnc(N)c2c1cnn2C]

Embodiment 106. The compound of any one of embodiments 36-38, or a pharmaceutically acceptable salt thereof, wherein Ring A is

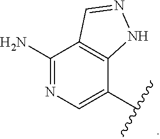 [SMILES: *c1cnc(N)c2cn[nH]c12]

Embodiment 107. The compound of any one of embodiments 36-38, or a pharmaceutically acceptable salt thereof, wherein the compound is of Formula (A_1b):

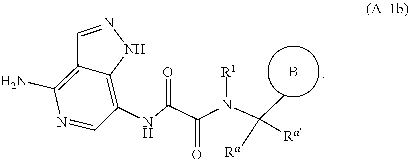

Embodiment 108. The compound of embodiment 37, or a pharmaceutically acceptable salt thereof, wherein the compound is of Formula (A_4b):

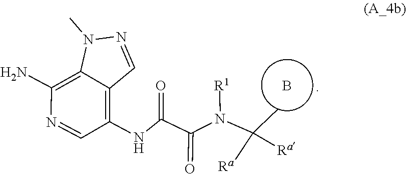

Embodiment 109. The compound of any one of embodiments 36-38, or a pharmaceutically acceptable salt thereof, wherein the compound is of Formula (I_1b):

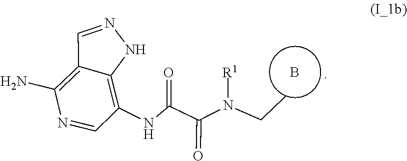

Embodiment 110. The compound of embodiment 37, or a pharmaceutically acceptable salt thereof, wherein the compound is of Formula I_4b):

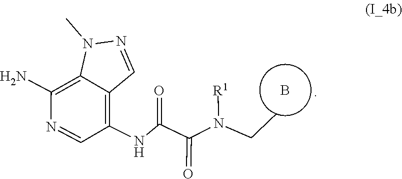

Embodiment 111. The compound of embodiment 36, or a pharmaceutically acceptable salt thereof, wherein the compound is of Formula (A_5c):

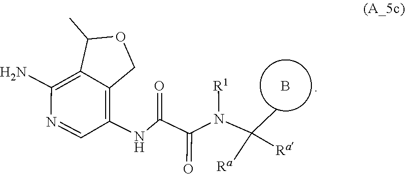

Embodiment 112. The compound of embodiment 36, or a pharmaceutically acceptable salt thereof, wherein the compound is of Formula (I_5c):

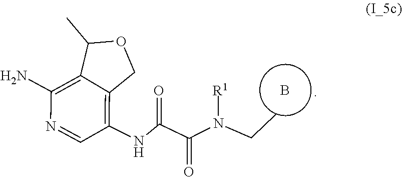

Embodiment 113. The compound of embodiment 36, or a pharmaceutically acceptable salt thereof, wherein the compound is of Formula (A_5d):

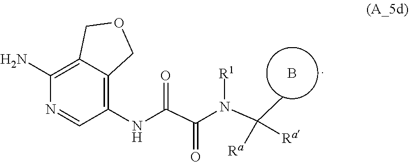

Embodiment 114. The compound of embodiment 36, or a pharmaceutically acceptable salt thereof, wherein the compound is of Formula (I_5d):

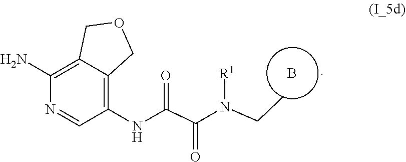

Embodiment 115. The compound of any one of embodiments 1-12, 36-38 and 43-47 or a pharmaceutically acceptable salt thereof, wherein Ring A is

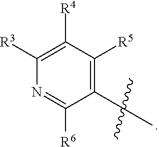

Embodiment 116. The compound of any one of embodiments 1-12, 36-38, 43-47 and 115, or a pharmaceutically acceptable salt thereof, wherein R⁵ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5}), and —OC(═O)N(R^{a5})₂.

Embodiment 117. The compound of embodiment 116, or a pharmaceutically acceptable salt thereof, wherein R⁵ is selected from the group consisting of H, halo, —CN, —C₁-C₆ alkyl, —C₁-C₆ haloalkyl and —N(R^{a5})₂.

Embodiment 118. The compound of embodiment 116 or 117, or a pharmaceutically acceptable salt thereof, wherein R^{a5} is selected from the group consisting of H and C₁-C₆ alkyl (e.g., -Me, -Et, —Pr, -iPr, -nBu, -tBu, -sec-Bu, -iso-Bu).

Embodiment 119. The compound of embodiment 116, or a pharmaceutically acceptable salt thereof, wherein R⁵ is selected from the group consisting of —C₁-C₆ alkyl (e.g., -Me, -Et, —Pr, -iPr, -nBu, -tBu, -sec-Bu, -iso-Bu), —OH, —O—(C₁-C₆ alkyl)(e.g., —OCH₃), —NH₂, —NH—(C₁-C₆ alkyl)(e.g., —NHCH₃) and —N—(C₁-C₆ alkyl)₂ (e.g, —N(CH₃)₂).

Embodiment 120. The compound of embodiment 116, or a pharmaceutically acceptable salt thereof, wherein R⁵ is selected from the group consisting of H and -Me.

Embodiment 121. The compound of embodiment 116, or a pharmaceutically acceptable salt thereof, wherein R⁵ is —H.

Embodiment 122. The compound of any one of embodiments 1-12, 36-38, 43-47 and 115-121, or a pharmaceutically acceptable salt thereof, wherein R⁶ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂ and —OC(═O)N(R^{a6})₂.

Embodiment 123. The compound of embodiment 122, or a pharmaceutically acceptable salt thereof, wherein R⁶ is selected from the group consisting of H, halo, —CN, —C₁-C₆ alkyl, —C₁-C₆ haloalkyl and —N(R^{a6})₂.

Embodiment 124. The compound of embodiment 122 or 123, or a pharmaceutically acceptable salt thereof, wherein each R^{a6} is independently selected from the group consisting of H and —C₁-C₆ alkyl (e.g., -Me, -Et, —Pr, -iPr, -nBu, -tBu, -sec-Bu, -iso-Bu).

Embodiment 125. The compound of embodiment 122, or a pharmaceutically acceptable salt thereof, wherein R⁶ is selected from the group consisting of —C₁-C₆ alkyl (e.g., -Me, -Et, —Pr, -iPr, -nBu, -tBu, -sec-Bu, -iso-Bu), —OH, —O—(C₁-C₆ alkyl)(e.g., —OCH₃), —NH₂, —NH—(C₁-C₆ alkyl)(e.g., —NHCH₃) and —N—(C₁-C₆ alkyl)₂ (e.g, —N(CH₃)₂).

Embodiment 126. The compound of embodiment 122, or a pharmaceutically acceptable salt thereof, wherein R⁶ is H.

Embodiment 127. The compound of any one of embodiments 1-12, 36-38, 43-47 and 115, or a pharmaceutically acceptable salt thereof, wherein Ring A is:

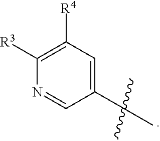

Embodiment 128. The compound of any one of embodiments 36-38, or a pharmaceutically acceptable salt thereof, wherein the compound is of Formula (A_2a):

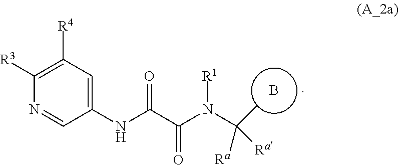

Embodiment 129. The compound of any one of embodiments 36-38, or a pharmaceutically acceptable salt thereof, wherein the compound is of Formula (1 2a):

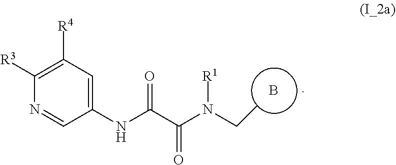

Embodiment 130. The compound of any one of embodiments 1-12, 36-38, 43-47 and 115-129, or a pharmaceutically acceptable salt thereof, wherein R³ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a3}, —N(R^{a3})₂, —C(═O)R^{a3}, —C(═O)OR^{a3}, -—NR^{a3}C(═O)R^{a3}, —NR^{a3}C(═O)OR^{a3}, —C(═O)N(R^{a3})₂ and —OC(═O)N(R^{a3})₂.

Embodiment 131. The compound of embodiment 130, or a pharmaceutically acceptable salt thereof, wherein R³ is selected from the group consisting of H, halo, —CN, —C₁-C₆ alkyl, —C₁-C₆ haloalkyl, —OR^{a3} and —N(R^{a3})₂.

Embodiment 132. The compound of embodiment 130, or a pharmaceutically acceptable salt thereof, wherein R³ is selected from the group consisting of H, —C₁-C₆ alkyl, —C₁-C₆ haloalkyl, —OR^{a3} and —N(R^{a3})₂.

Embodiment 133. The compound of embodiment 130, or a pharmaceutically acceptable salt thereof, wherein R³ is selected from the group consisting of H, —OR^{a3} and —N(R^{a3})₂.

Embodiment 134. The compound of any one of embodiments 130 to 133, or a pharmaceutically acceptable salt thereof, wherein each R^{a3} is independently selected from the group consisting of H, —C₁-C₆ alkyl (e.g., -Me, -Et, —Pr, —Pr, -nBu, -^{t}Bu, -sec-Bu, -iso-Bu) and —C₁-C₆ haloalkyl (e.g., —CHF₂, —CF₃).

Embodiment 135. The compound of embodiment 130, or a pharmaceutically acceptable salt thereof, wherein R³ is selected from the group consisting of H, —C₁-C₆ alkyl (e.g., -Me, -Et, —Pr, -iPr, -nBu, -tBu, -sec-Bu, -iso-Bu), —C₁-C₆ alkyl (e.g., —CF₃, —CHF₂), —OH, —O—(C₁-C₆ alkyl)(e.g., —OCH₃, -OEt), —O—(C₁-C₆ haloalkyl)(e.g., —OCF₃, —OCHF₂), —NH₂, —NH—(C₁-C₆ alkyl)(e.g., —NHCH₃) and —N—(C₁-C₆ alkyl)₂ (e.g, —N(CH₃)₂).

Embodiment 136. The compound of embodiment 130, or a pharmaceutically acceptable salt thereof, wherein R³ is selected from the group consisting of H, -Me, —CHF₂, —OCH₃ and —NH₂.

Embodiment 137. The compound of embodiment 130, or a pharmaceutically acceptable salt thereof, wherein R³ is selected from the group consisting of H, —NH₂ and —OCH₃.

Embodiment 138. The compound of embodiment 130, or a pharmaceutically acceptable salt thereof, wherein R³ is H.

Embodiment 139. The compound of embodiment 130, or a pharmaceutically acceptable salt thereof, wherein R³ is —OCH₃.

Embodiment 140. The compound of embodiment 130, or a pharmaceutically acceptable salt thereof, wherein R³ is —NH₂.

Embodiment 141. The compound of any one of embodiments 1-12, 36-38, 43-47 and 115-140, or a pharmaceutically acceptable salt thereof, wherein R⁴ is selected from the group consisting of halo, —CN, —C₁-C₆ alkyl, —C₁-C₆ heteroalkyl, —C₁-C₆ haloalkyl, —C₃-C₉ cycloalkyl (e.g., cyclopropyl), 3-10 membered heterocyclyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂.

Embodiment 142. The compound of embodiment 141, or a pharmaceutically acceptable salt thereof, wherein R⁴ is selected from the group consisting of halo, —CN, -C1-C6 alkyl, —C₁-C₆ haloalkyl, 3-10 membered heterocyclyl (e.g., oxetanyl), —C₃-C₉ cycloalkyl (e.g., cyclopropyl), —OR^{a4}, —N(R^{a4})₂, —C(═O)R^{a4} and —C(═O)N(R^{a4})₂.

Embodiment 143. The compound of embodiment 141, or a pharmaceutically acceptable salt thereof, wherein R⁴ is selected from the group consisting of halo, —C₁-C₆ alkyl, —C₁-C₆ haloalkyl, 3-10 membered heterocyclyl (e.g., oxetanyl), —C₃-C₉ cycloalkyl (e.g., cyclopropyl), —OR^{a4}, —C(═O)R^{a4} and —C(═O)N(R^{a4})₂.

Embodiment 144. The compound of embodiment 141, or a pharmaceutically acceptable salt thereof, wherein R⁴ is selected from the group consisting of —C₁-C₆ alkyl, 3-10 membered heterocyclyl (e.g., oxetanyl), —C₃-C₉ cycloalkyl (e.g., cyclopropyl) and —C(═O)N(R^{a4})₂.

Embodiment 145. The compound of any one of embodiments 141 to 144, or a pharmaceutically acceptable salt thereof, wherein each R^{a4} is independently selected from the group consisting of H and —C₁-C₆ alkyl (e.g., -Me, -Et, —Pr, -iPr, -nBu, -tBu, -sec-Bu, -iso-Bu).

Embodiment 146. The compound of embodiment 141, or a pharmaceutically acceptable salt thereof, wherein R⁴ is selected from the group consisting of —C₁-C₆ alkyl (e.g., -Me, -Et, —Pr, -iPr, -nBu, -tBu, -sec-Bu, -iso-Bu), 3-10 membered heterocyclyl (e.g., oxetan-3-yl), —C₃-C₉ cycloalkyl (e.g., cyclopropyl) and —C(═O)NH₂.

Embodiment 147. The compound of embodiment 141, or a pharmaceutically acceptable salt thereof, wherein R⁴ is selected from the group consisting of -Me, -Et, oxetan-3-yl, cyclopropyl and —C(═O)NH₂.

Embodiment 148. The compound of embodiment 141, or a pharmaceutically acceptable salt thereof, wherein R⁴ is selected from the group consisting of -Me, -Et, cyclopropyl and —C(═O)NH₂.

Embodiment 149. The compound of embodiment 141, or a pharmaceutically acceptable salt thereof, wherein R⁴ is —C(═O)NH₂.

Embodiment 150. The compound of embodiment 141, or a pharmaceutically acceptable salt thereof, wherein R⁴ is selected from the group consisting of -Me, -Et, oxetan-3-yl and cyclopropyl.

Embodiment 151. The compound of embodiment 141, or a pharmaceutically acceptable salt thereof, wherein R⁴ is selected from the group consisting of -Me, -Et and cyclopropyl.

Embodiment 152. The compound of any one of embodiments 1-12, 36-38, 43-47 and 115-127, or a pharmaceutically acceptable salt thereof, wherein R³ is —OCH₃ and R⁴ is —C(═O)NH₂.

Embodiment 153. The compound of any one of embodiments 1-12, 36-38, 43-47 and 115-127, or a pharmaceutically acceptable salt thereof, wherein R³ is —NH₂ and R⁴ is selected from the group consisting of -Me, -Et, oxetan-3-yl and cyclopropyl.

Embodiment 154. The compound of any one of embodiments 1-12, 36-38, 43-47 and 115-127, or a pharmaceutically acceptable salt thereof, wherein R³ is —NH₂ and R⁴ is selected from the group consisting of -Me, -Et and cyclopropyl.

Embodiment 155. The compound of embodiment 36 or 37, or a pharmaceutically acceptable salt thereof, wherein Ring A is selected from the group consisting of:

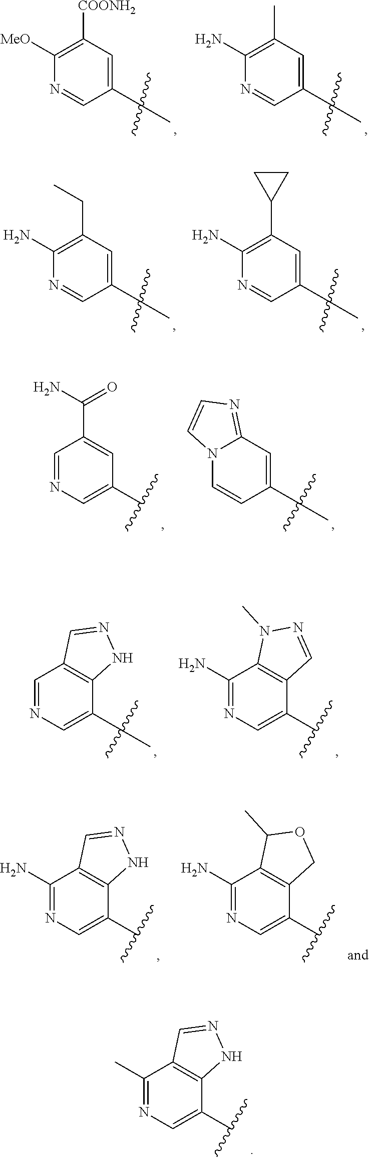

Embodiment 156. The compound of embodiment 36 or 37, or a pharmaceutically acceptable salt thereof, wherein Ring A is selected from the group consisting of:

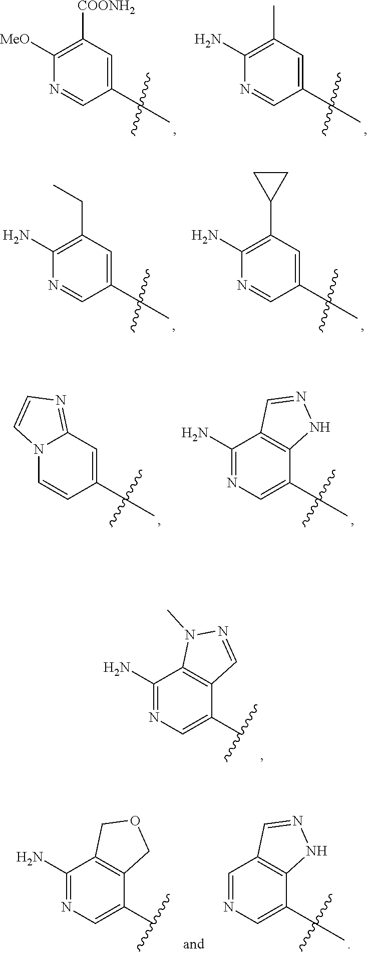

Embodiment 157. The compound of any one of embodiments 36-38, or a pharmaceutically acceptable salt thereof, wherein Ring A is selected from the group consisting of:

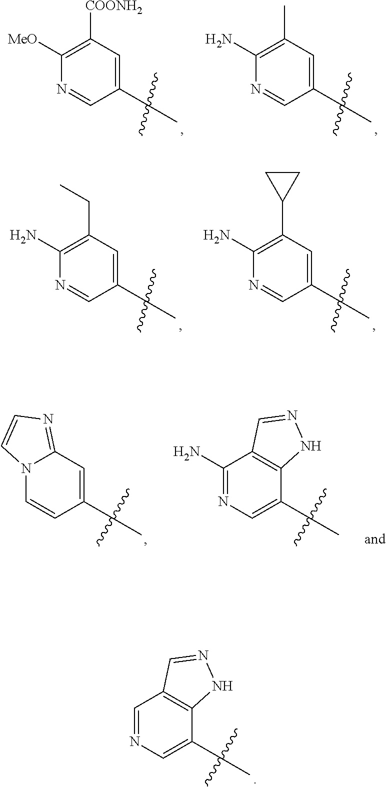

Embodiment 158. The compound of any one of embodiments 1 to 157, or a pharmaceutically acceptable salt thereof, wherein Ring B is selected from the group consisting of C₆-C₁₀ aryl (e.g., phenyl, naphthalenyl), 5-6 membered monocyclic heteroaryl, and 8-10 membered bicyclic heteroaryl, each optionally substituted at any available position.

Embodiment 159. The compound of any one of embodiments 1 to 157, or a pharmaceutically acceptable salt thereof, wherein Ring B is selected from the group consisting of C₆-C₁₀ aryl and 5-6 membered monocyclic heteroaryl wherein the aryl and heteroaryl are optionally substituted at any available position.

Embodiment 160. The compound of any one of embodiments 1 to 157, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl, chromanyl and 1,2,3,4-tetrahydro-1,8-naphthyridinyl), each optionally substituted.

Embodiment 161. The compound of any one of embodiments 1 to 157, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl), each optionally substituted.

Embodiment 162. The compound of any one of embodiments 1 to 157, or a pharmaceutically acceptable salt thereof, wherein Ring B is selected from the group consisting of pyridinyl, pyrimidinyl, isoquinolinyl, pyrazolyl, chromanyl and phenyl, each optionally substituted.

Embodiment 163. The compound of any one of embodiments 1 to 157, or a pharmaceutically acceptable salt thereof, wherein Ring B is selected from the group consisting of pyridinyl and phenyl, each optionally substituted.

Embodiment 164. The compound of any one of embodiments 1 to 157, or a pharmaceutically acceptable salt thereof, wherein Ring B is selected from the group consisting of pyridinyl, pyrimidinyl and phenyl, each optionally substituted.

Embodiment 165. The compound of any one of embodiments 1 to 157, or a pharmaceutically acceptable salt thereof, wherein Ring B is selected from the group consisting of pyrazol-5-yl, pyrazol-1-yl, thiophen-2-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, isoquinolin-1-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl, chroman-5-yl and thiazolo[5,4-b]pyridin-6-yl), each optionally substituted.

Embodiment 166. The compound of any one of embodiments 1 to 157, or a pharmaceutically acceptable salt thereof, wherein Ring B is selected from the group consisting of pyrazol-5-yl, thiophen-2-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted.

Embodiment 167. The compound of any one of embodiments 1 to 157, or a pharmaceutically acceptable salt thereof, wherein Ring B is selected from the group consisting of pyridin-2-yl, pyridin-3-yl, pyridin-4-yl, pyrimidin-2-yl, pyrazol-5-yl, pyrazol-1-yl, isoquinolin-1-yl, chroman-5-yl and phenyl, each optionally substituted.

Embodiment 168. The compound of any one of embodiments 1 to 157, or a pharmaceutically acceptable salt thereof, wherein Ring B is selected from the group consisting of pyridin-2-yl and phenyl, each optionally substituted.

Embodiment 169. The compound of any one of embodiments 1 to 157, or a pharmaceutically acceptable salt thereof, wherein Ring B is selected from the group consisting of pyridin-2-yl, pyridin-3-yl, pyridin-4-yl, pyrimidin-2-yl and phenyl, each optionally substituted.

Embodiment 170. The compound of any one of embodiments 1 to 157, or a pharmaceutically acceptable salt thereof, wherein Ring B is C₆-C₁₀ aryl (e.g., naphthalenyl, phenyl), wherein the aryl is optionally substituted at any available position.

Embodiment 171. The compound of any one of embodiments 1 to 157, or a pharmaceutically acceptable salt thereof, wherein Ring B is naphthalenyl or phenyl, each optionally substituted at any available position.

Embodiment 172. The compound of any one of embodiments 1 to 157, or a pharmaceutically acceptable salt thereof, wherein Ring B is phenyl, optionally substituted at any available position.

Embodiment 173. The compound of any one of embodiments 1 to 157, or a pharmaceutically acceptable salt thereof, wherein Ring B is 5-6 membered monocyclic heteroaryl wherein the heteroaryl is optionally substituted at any available position.

Embodiment 174. The compound of any one of embodiments 1 to 157, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, pyridinyl, pyrimidinyl, pyrazinyl and pyridazinyl, each optionally substituted.

Embodiment 175. The compound of any one of embodiments 1 to 157, or a pharmaceutically acceptable salt thereof, wherein Ring B is selected from the group consisting of pyridinyl and pyrimidinyl, each, optionally substituted.

Embodiment 176. The compound of any one of embodiments 1 to 157, or a pharmaceutically acceptable salt thereof, wherein Ring B is selected from the group consisting of pyrazol-5-yl, pyrazol-1-yl, thiophene-2-yl, thiophen-3-yl, oxazol-5-yl, thiazol-5-yl, pyridin-2-yl, pyridin-3-yl, pyridin-4-yl and pyrimidin-2-yl, each optionally substituted.

Embodiment 177. The compound of any one of embodiments 1 to 157, or a pharmaceutically acceptable salt thereof, wherein Ring B is selected from the group consisting of pyrazol-5-yl, thiophene-2-yl, thiophen-3-yl, oxazol-5-yl, thiazol-5-yl, pyridin-2-yl, pyridin-3-yl, pyridin-4-yl and pyrimidin-2-yl, each optionally substituted.

Embodiment 178. The compound of any one of embodiments 1 to 157, or a pharmaceutically acceptable salt thereof, wherein Ring B is selected from the group consisting of pyridin-2-yl, pyridin-3-yl, pyridin-4-yl, pyrimidin-2-yl, each optionally substituted.

Embodiment 179. The compound of any one of embodiments 1 to 157, or a pharmaceutically acceptable salt thereof, wherein Ring B is selected from the group consisting of pyridin-2-yl and pyrimidin-2-yl, each optionally substituted.

Embodiment 180. The compound of any one of embodiments 1 to 157, or a pharmaceutically acceptable salt thereof, wherein Ring B is optionally substituted pyridin-2-yl.

Embodiment 181. The compound of any one of embodiments 1 to 157, or a pharmaceutically acceptable salt thereof, wherein Ring B is optionally substituted pyridin-3-yl.

Embodiment 182. The compound of any one of embodiments 1 to 157, or a pharmaceutically acceptable salt thereof, wherein Ring B is optionally substituted pyridin-4-yl.

Embodiment 183. The compound of any one of embodiments 1 to 157, or a pharmaceutically acceptable salt thereof, wherein Ring B is optionally substituted pyrimidin-2-yl.

Embodiment 184. The compound of any one of embodiments 1 to 157, or a pharmaceutically acceptable salt thereof, wherein Ring B is an 8-10 membered bicyclic heteroaryl, wherein the bicyclic heteroaryl is optionally substituted at any available position.

Embodiment 185. The compound of any one of embodiments 1 to 157, or a pharmaceutically acceptable salt thereof, wherein Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl, chromanyl and 1,2,3,4-tetrahydro-1,8-naphthyridinyl, each optionally substituted.

Embodiment 186. The compound of any one of embodiments 1 to 157, or a pharmaceutically acceptable salt thereof, wherein Ring B is selected from the group consisting of isoquinolinyl and chromanyl, each optionally substituted.

Embodiment 187. The compound of any one of embodiments 1 to 157, or a pharmaceutically acceptable salt thereof, wherein Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each optionally substituted.

Embodiment 188. The compound of any one of embodiments 1 to 157, or a pharmaceutically acceptable salt thereof, wherein Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-1-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl, chroman-5-yl and thiazolo[5,4-b]pyridin-6-yl, each optionally substituted.

Embodiment 189. The compound of any one of embodiments 1 to 157, or a pharmaceutically acceptable salt thereof, wherein Ring B is selected from the group consisting of isoquinolin-1-yl and chroman-5-yl, each optionally substituted.

Embodiment 190. The compound of any one of embodiments 1 to 157, or a pharmaceutically acceptable salt thereof, wherein Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl, each optionally substituted.

Embodiment 191. The compound of any one of embodiments 1 to 190, or a pharmaceutically acceptable salt thereof, wherein each Ring B is substituted at any available position with 0, 1, 2 or 3 instances of R⁷, wherein:
each R⁷ is independently selected from the group consisting of -D, ═O, —CN, halo, —SF₅, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a7}, —N(R^{a7})₂, —C(═O)R^{a7}, —C(═O)OR^{a7}, —NR^{a7}C(═O)R^{a7}, —NR^{a7}C(═O)OR^{a7}, —C(═O)N(R^{a7})₂, —OC(═O)R^{a7}, —OC(═O)N(R^{a7})₂, —S(═O)R^{a7}, —S(═O)₂R^{a7}, —SR^{a7}, —S(═O)(═NR^{a7})R^{a7}, —NR^{a7}S(═O)₂R^{a7} and —S(═O)₂N(R^{a7})₂, wherein each alkyl, cycloalkyl, heterocyclyl, aryl, heteroaryl, cycloalkylalkyl, heterocyclylalkyl, arylalkyl and heteroarylalkyl of R⁷ is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof);
each R^{a7} is independently H; —C₁-C₆ alkyl; —C₁-C₆ haloalkyl; —C₁-C₆ heteroalkyl substituted with 0 or 1 instance of ═O; C₃-C₉ cycloalkyl; or 3-10 membered heterocyclyl substituted with 0 or 1 instances of ═O, -Me or a combination thereof.

Embodiment 192. The compound of any one of embodiments 1 to 190 wherein each Ring B is substituted at any available position with 0, 1, 2 or 3 instances of R⁷, wherein:
each R⁷ is independently selected from the group consisting of -D, ═O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a7}, —N(R^{a7})₂, —C(═O)R^{a7}, —C(═O)OR^{a7}, —NR^{a7}C(═O)R^{a7}, -NR^{a7}C(═O)OR^{a7}, —C(═O)N(R^{a7})₂, —OC(═O)R^{a7}, —OC(═O)N(R^{a7})₂, —S(═O)R^{a7}, —S(═O)₂R^{a7}, —SR^{a7}, —S(═O)(═NR^{a7})R^{a7}, —NR^{a7}S(═O)₂R^{a7} and —S(═O)₂N(R^{a7})₂, wherein each alkyl, cycloalkyl, heterocyclyl, aryl, heteroaryl, cycloalkylalkyl, heterocyclylalkyl, arylalkyl and heteroarylalkyl of R⁷ is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof); each R^{a7} is independently H; —C₁-C₆ alkyl; —C₁-C₆ haloalkyl; —C₁-C₆ heteroalkyl substituted with 0 or 1 instance of —O; C₃-C₉ cycloalkyl; or 3-10 membered heterocyclyl substituted with 0 or 1 instances of ═O, -Me or a combination thereof.

Embodiment 193. The compound of embodiment 191 or 192, or a pharmaceutically acceptable salt thereof, wherein Ring B is selected from the group consisting of C₆-C₁₀ aryl (e.g., phenyl, naphthalenyl), 5-6 membered monocyclic heteroaryl, and 8-10 membered bicyclic heteroaryl, each substituted at any available position with 0, 1, 2 or 3 instances of R⁷.

Embodiment 194. The compound of embodiment 191 or 192, or a pharmaceutically acceptable salt thereof, wherein Ring B is selected from the group consisting of C₆-C₁₀ aryl and 5-6 membered monocyclic heteroaryl wherein the aryl and heteroaryl are substituted at any available position with 0, 1, 2 or 3 instances of R⁷.

Embodiment 195. The compound of embodiment 191, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazoly], [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl, chromanyl and 1,2,3,4-tetrahydro-1,8-naphthyridinyl), each substituted at any available position with 0, 1, 2 or 3 instances of R⁷.

Embodiment 196. The compound of embodiment 191 or 192, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazoly], [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl), each substituted at any available position with 0, 1, 2 or 3 instances of R⁷.

Embodiment 197. The compound of embodiment 191, or a pharmaceutically acceptable salt thereof, wherein Ring B is selected from the group consisting of pyridinyl, pyrimidinyl, isoquinolinyl, pyrazolyl, chromanyl and phenyl, each substituted at any available position with 0, 1, 2 or 3 instances of R⁷.

Embodiment 198. The compound of embodiment 191 or 192, or a pharmaceutically acceptable salt thereof, wherein Ring B is selected from the group consisting of pyridinyl and phenyl, each substituted at any available position with 0, 1, 2 or 3 instances of R⁷.

Embodiment 199. The compound of embodiment 191 or 192, or a pharmaceutically acceptable salt thereof, wherein Ring B is selected from the group consisting of pyridinyl, pyrimidinyl and phenyl, each substituted at any available position with 0, 1, 2 or 3 instances of R⁷.

Embodiment 200. The compound of embodiment 191, or a pharmaceutically acceptable salt thereof, wherein Ring B is selected from the group consisting of pyrazol-5-yl, pyrazol-1-yl, thiophen-2-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, isoquinolin-1-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl, chroman-5-yl and thiazolo[5,4-b]pyridin-6-yl), each substituted at any available position with 0, 1, 2 or 3 instances of R⁷.

Embodiment 201. The compound of embodiment 191 or 192, or a pharmaceutically acceptable salt thereof, wherein Ring B is selected from the group consisting of pyrazol-5-yl, thiophen-2-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each substituted at any available position with 0, 1, 2 or 3 instances of R⁷.

Embodiment 202. The compound of embodiment 191, or a pharmaceutically acceptable salt thereof, wherein Ring B is selected from the group consisting of pyridin-2-yl, pyridin-3-yl, pyridin-4-yl, pyrimidin-2-yl, pyrazol-5-yl, pyrazol-1-yl, isoquinolin-1-yl, chroman-5-yl and phenyl, each substituted at any available position with 0, 1, 2 or 3 instances of R⁷.

Embodiment 203. The compound of embodiment 191 or 192, or a pharmaceutically acceptable salt thereof, wherein Ring B is selected from the group consisting of pyridin-2-yl and phenyl, each substituted at any available position with 0, 1, 2 or 3 instances of R⁷.

Embodiment 204. The compound of embodiment 191 or 192, or a pharmaceutically acceptable salt thereof, wherein Ring B is selected from the group consisting of pyridin-2-yl, pyridin-3-yl, pyridin-4-yl, pyrimidin-2-yl and phenyl, each substituted at any available position with 0, 1, 2 or 3 instances of R⁷.

Embodiment 205. The compound of embodiment 191 or 192, or a pharmaceutically acceptable salt thereof, wherein Ring B is C₆-C₁₀ aryl (e.g., naphthalenyl, phenyl), wherein the aryl is substituted at any available position with 0, 1, 2 or 3 instances of R⁷.

Embodiment 206. The compound of embodiment 191 or 192, or a pharmaceutically acceptable salt thereof, wherein Ring B is naphthalenyl or phenyl, each substituted at any available position with 0, 1, 2 or 3 instances of R⁷.

Embodiment 207. The compound of embodiment 191 or 192, or a pharmaceutically acceptable salt thereof, wherein Ring B is phenyl, substituted at any available position with 0, 1, 2 or 3 instances of R⁷.

Embodiment 208. The compound of embodiment 191 or 192, or a pharmaceutically acceptable salt thereof, wherein Ring B is 5-6 membered monocyclic heteroaryl wherein the heteroaryl is substituted at any available position with 0, 1, 2 or 3 instances of R⁷.

Embodiment 209. The compound of embodiment 191 or 192, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, pyridinyl, pyrimidinyl, pyrazinyl and pyridazinyl, each substituted at any available position with 0, 1, 2 or 3 instances of R⁷.

Embodiment 210. The compound of embodiment 191 or 192, or a pharmaceutically acceptable salt thereof, wherein Ring B is selected from the group consisting of pyridinyl and pyrimidinyl, each substituted at any available position with 0, 1, 2 or 3 instances of R⁷.

Embodiment 211. The compound of embodiment 191, or a pharmaceutically acceptable salt thereof, wherein Ring B is selected from the group consisting of pyrazol-5-yl, pyrazol-1-yl, thiophene-2-yl, thiophen-3-yl, oxazol-5-yl, thiazol-5-yl, pyridin-2-yl, pyridin-3-yl, pyridin-4-yl and pyrimidin-2-yl, each substituted at any available position with 0, 1, 2 or 3 instances of R⁷.

Embodiment 212. The compound of embodiment 191 or 192, or a pharmaceutically acceptable salt thereof, wherein Ring B is selected from the group consisting of pyrazol-5-yl, thiophene-2-yl, thiophen-3-yl, oxazol-5-yl, thiazol-5-yl, pyridin-2-yl, pyridin-3-yl, pyridin-4-yl and pyrimidin-2-yl, substituted at any available position with 0, 1, 2 or 3 instances of R⁷.

Embodiment 213. The compound of embodiment 191 or 192, or a pharmaceutically acceptable salt thereof, wherein Ring B is selected from the group consisting of pyridin-2-yl, pyridin-3-yl, pyridin-4-yl and pyrimidin-2-yl, each substituted at any available position with 0, 1, 2 or 3 instances of R⁷.

Embodiment 214. The compound of embodiment 191 or 192, or a pharmaceutically acceptable salt thereof, wherein Ring B is selected from the group consisting of pyridin-2-yl and pyrimidin-2-yl, each substituted at any available position with 0, 1, 2 or 3 instances of R⁷. Embodiment 215. The compound of embodiment 191, or a pharmaceutically

acceptable salt thereof, wherein Ring B is pyridin-2-yl substituted at any available position with 0, 1, 2 or 3 instances of R⁷.

Embodiment 216. The compound of embodiment 191 or 192, or a pharmaceutically acceptable salt thereof, wherein Ring B is pyridin-3-yl substituted at any available position with 0, 1, 2 or 3 instances of R⁷.

Embodiment 217. The compound of embodiment 191 or 192, or a pharmaceutically acceptable salt thereof, wherein Ring B is pyridin-4-yl substituted at any available position with 0, 1, 2 or 3 instances of R⁷.

Embodiment 218. The compound of embodiment 191 or 192, or a pharmaceutically acceptable salt thereof, wherein Ring B is pyrimidin-2-yl substituted at any available position with 0, 1, 2 or 3 instances of R⁷.

Embodiment 219. The compound of embodiment 191 or 192, or a pharmaceutically acceptable salt thereof, wherein Ring B is an 8-10 membered bicyclic heteroaryl, wherein the bicyclic heteroaryl is substituted at any available position with 0, 1, 2 or 3 instances of R⁷.

Embodiment 220. The compound of embodiment 191, or a pharmaceutically acceptable salt thereof, wherein Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl, chromanyl and 1,2,3,4-tetrahydro-1,8-naphthyridinyl, each substituted at any available position with 0, 1, 2 or 3 instances of R7

Embodiment 221. The compound of embodiment 191, or a pharmaceutically acceptable salt thereof, wherein Ring B is selected from the group consisting of isoquinolinyl and chromanyl, each substituted at any available position with 0, 1, 2 or 3 instances of R⁷.

Embodiment 222. The compound of embodiment 191 or 192, or a pharmaceutically acceptable salt thereof, wherein Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each substituted at any available position with 0, 1, 2 or 3 instances of R⁷.

Embodiment 223. The compound of embodiment 191, or a pharmaceutically acceptable salt thereof, wherein Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-1-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl, chroman-5-yl and thiazolo[5,4-b]pyridin-6-yl, each substituted at any available position with 0, 1, 2 or 3 instances of R⁷.

Embodiment 224. The compound of embodiment 191, or a pharmaceutically acceptable salt thereof, wherein Ring B is selected from the group consisting of isoquinolin-1-yl and chroman-5-yl, each substituted at any available position with 0, 1, 2 or 3 instances of R⁷.

Embodiment 225. The compound of embodiment 191 or 192, or a pharmaceutically acceptable salt thereof, wherein Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl, each substituted at any available position with 0, 1, 2 or 3 instances of R⁷.

Embodiment 226. The compound of embodiment 191 or 192, or a pharmaceutically acceptable salt thereof, wherein Ring B is substituted with 0, 1 or 2 instances of R⁷.

Embodiment 227. The compound of embodiment 191 or 192, or a pharmaceutically acceptable salt thereof, wherein Ring B is substituted with 0 or 1 instances of R⁷.

Embodiment 228. The compound of embodiment 191 or 192, or a pharmaceutically acceptable salt thereof, wherein Ring B is substituted with 1 or 2 instances of R⁷.

Embodiment 229. The compound of embodiment 191, or a pharmaceutically acceptable salt thereof, wherein Ring B is selected from the group consisting of:

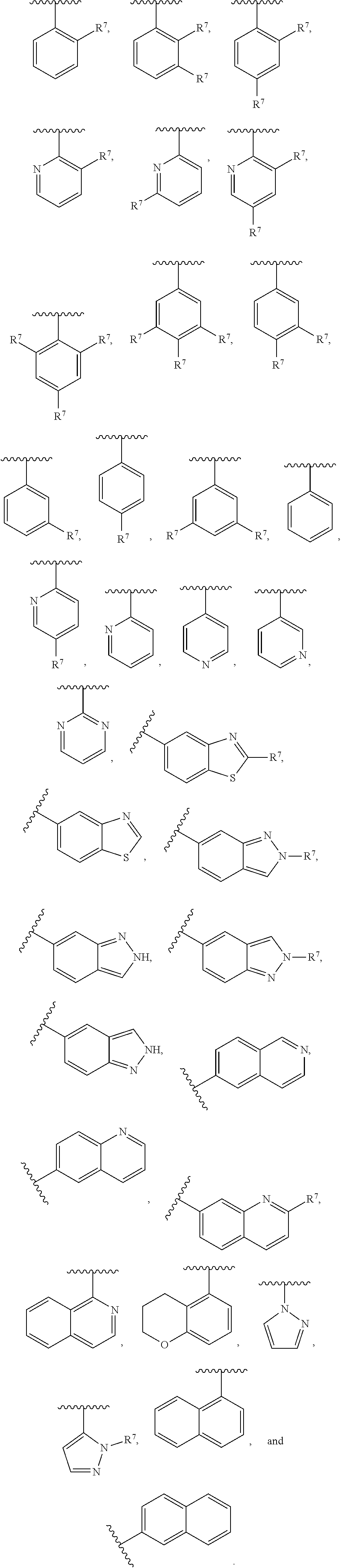

Embodiment 230. The compound of embodiment 191, or a pharmaceutically acceptable salt thereof, wherein Ring B is selected from the group consisting of:

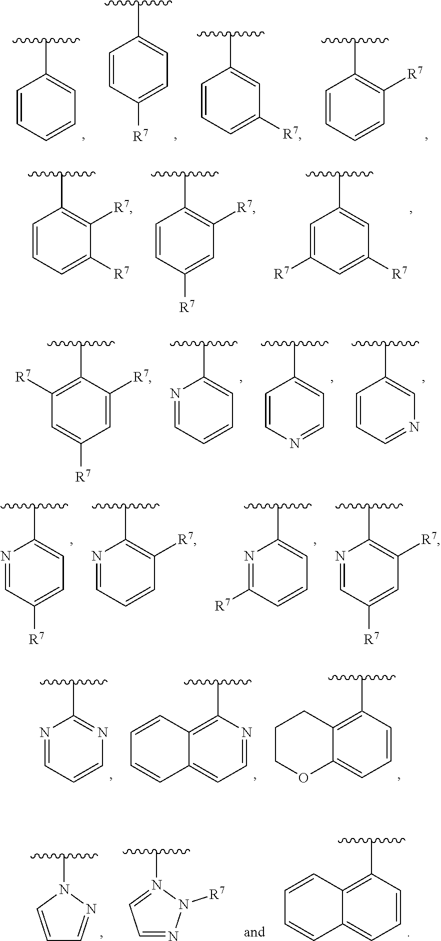

Embodiment 231. The compound of embodiment 191 or 192, or a pharmaceutically acceptable salt thereof, wherein Ring B is selected from the group consisting of:

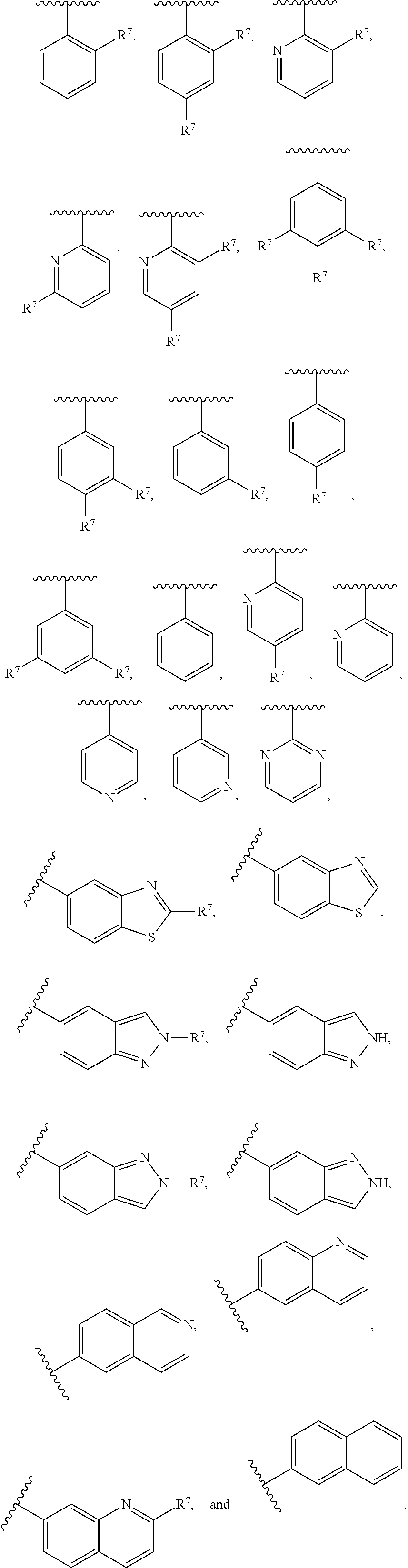

Embodiment 232. The compound of embodiment 191 or 192, or a pharmaceutically acceptable salt thereof, wherein Ring B is selected from the group consisting of:

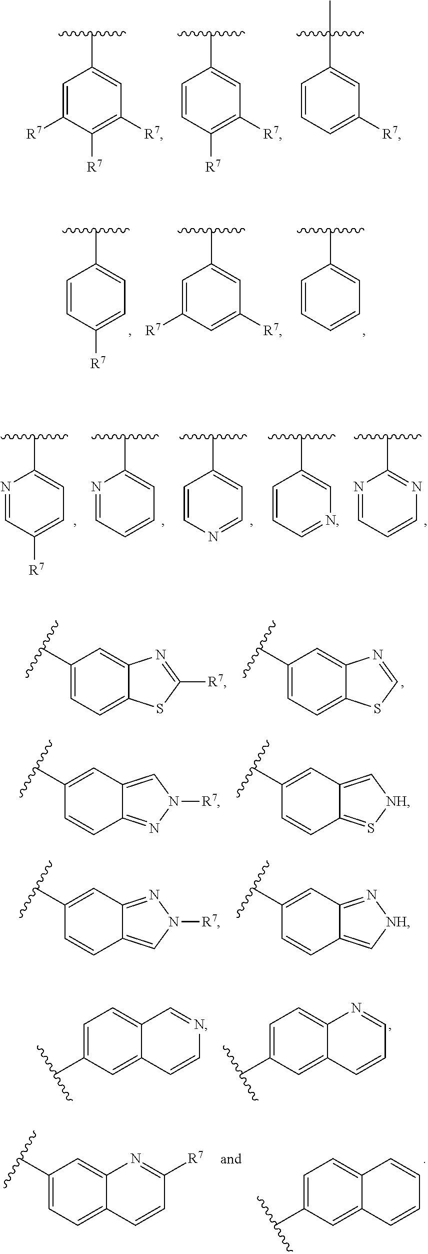

Embodiment 233. The compound of embodiment 191 or 192, or a pharmaceutically acceptable salt thereof, wherein Ring B is selected from the group consisting of:

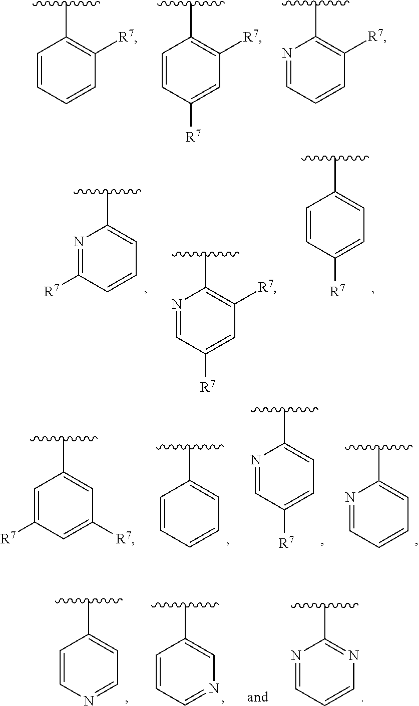

Embodiment 234. The compound of embodiment 191 or 192, or a pharmaceutically acceptable salt thereof, wherein Ring B is selected from the group consisting of:

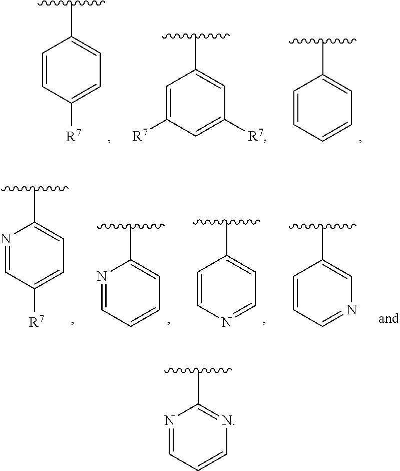

Embodiment 235. The compound of embodiment 191 or 192, or a pharmaceutically acceptable salt thereof wherein Ring B is selected from the group consisting of:

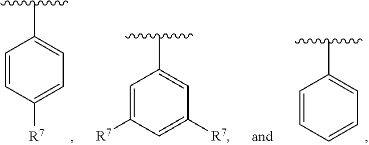

Embodiment 236. The compound of embodiment 191 or 192, or a pharmaceutically acceptable salt thereof, wherein Ring B is selected from the group consisting of:

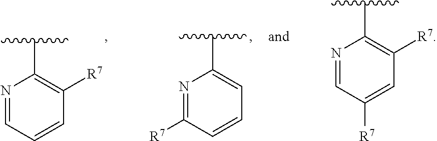

Embodiment 237. The compound of embodiment 191 or 192, or a pharmaceutically acceptable salt thereof, wherein Ring B is selected from the group consisting of:

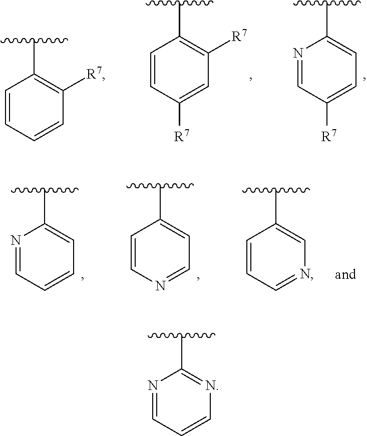

Embodiment 238. The compound of embodiment 191 or 192, or a pharmaceutically acceptable salt thereof, wherein Ring B is selected from the group consisting of:

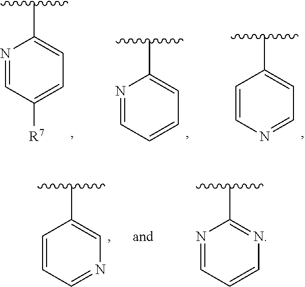

Embodiment 239. The compound of embodiment 191 or 192, or a pharmaceutically acceptable salt thereof, wherein Ring B is

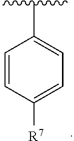

Embodiment 240. The compound of embodiment 191 or 192, or a pharmaceutically acceptable salt thereof, wherein Ring B is

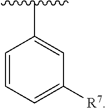

Embodiment 241. The compound of embodiment 191, or a pharmaceutically acceptable salt thereof, wherein Ring B is

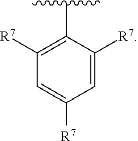

Embodiment 242. The compound of embodiment 191, or a pharmaceutically acceptable salt thereof, wherein Ring B is

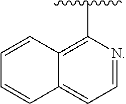 [SMILES: *c1nccc2ccccc12]

Embodiment 243. The compound of embodiment 191, or a pharmaceutically acceptable salt thereof, wherein Ring B is

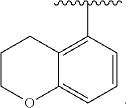 [SMILES: *c1cccc2c1CCCO2]

Embodiment 244. The compound of embodiment 191, or a pharmaceutically acceptable salt thereof, wherein Ring B is

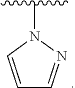 [SMILES: *n1cccn1 ; "."]

Embodiment 245. The compound of embodiment 191, or a pharmaceutically acceptable salt thereof, wherein Ring B is

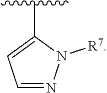

Embodiment 246. The compound of embodiment 191, or a pharmaceutically acceptable salt thereof, wherein Ring B is

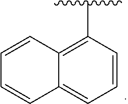 [SMILES: *c1cccc2ccccc12]

Embodiment 247. The compound of embodiment 191 or 192, or a pharmaceutically acceptable salt thereof, wherein Ring B is

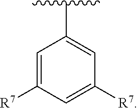

Embodiment 248. The compound of embodiment 191 or 192, or a pharmaceutically acceptable salt thereof, wherein Ring B is

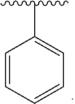 [SMILES: *c1ccccc1]

Embodiment 249. The compound of embodiment 191 or 192, or a pharmaceutically acceptable salt thereof, wherein Ring B is

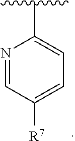

Embodiment 250. The compound of embodiment 191 or 192, or a pharmaceutically acceptable salt thereof, wherein Ring B is

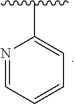 [SMILES: *c1ccccn1]

Embodiment 251. The compound of embodiment 191 or 192, or a pharmaceutically acceptable salt thereof, wherein Ring B is

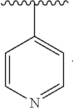 [SMILES: *c1ccncc1]

Embodiment 252. The compound of embodiment 191 or 192, or a pharmaceutically acceptable salt thereof, wherein Ring B is

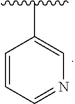 [SMILES: *c1cccnc1]

Embodiment 253. The compound of embodiment 191 or 192, or a pharmaceutically acceptable salt thereof, wherein Ring B is

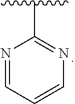 [SMILES: *c1ncccn1]

Embodiment 254. The compound of embodiment 191 or 192, or a pharmaceutically acceptable salt thereof, wherein Ring B is

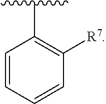

Embodiment 255. The compound of embodiment 191 or 192, or a pharmaceutically acceptable salt thereof, wherein Ring B is

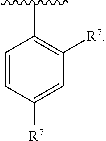

Embodiment 256. The compound of embodiment 191 or 192, or a pharmaceutically acceptable salt thereof, wherein Ring B is

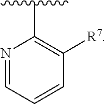

Embodiment 257. The compound of embodiment 191 or 192, or a pharmaceutically acceptable salt thereof, wherein Ring B is

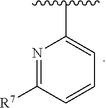

Embodiment 258. The compound of embodiment 191 or 192, or a pharmaceutically acceptable salt thereof, wherein Ring B is

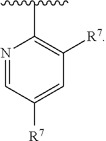

Embodiment 259. The compound of embodiment 191 or 192, or a pharmaceutically acceptable salt thereof, wherein the compound is of Formula (A-III):

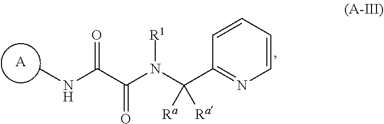

wherein the pyridinyl is substituted with 0, 1, 2 or 3 instances of R⁷.

Embodiment 260. The compound of embodiment 191 or 192, or a pharmaceutically acceptable salt thereof, wherein the compound is of Formula (III):

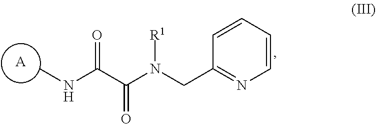

wherein the pyridinyl is substituted with 0, 1, 2 or 3 instances of R⁷.

Embodiment 261. The compound of embodiment 191 or 192, or a pharmaceutically acceptable salt thereof, wherein the compound is of Formula (A-III_1):

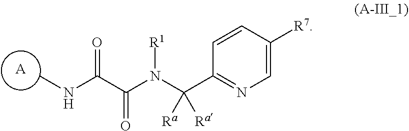

Embodiment 262. The compound of embodiment 191 or 192, or a pharmaceutically acceptable salt thereof, wherein the compound is of Formula (III_1):

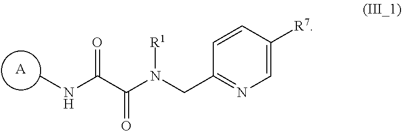

Embodiment 263. The compound of embodiment 191 or 192, or a pharmaceutically acceptable salt thereof, wherein the compound is of Formula (A-IV):

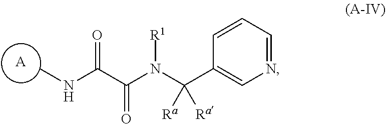

wherein the pyridinyl is substituted with 0, 1, 2 or 3 instances of R⁷.

Embodiment 264. The compound of embodiment 191 or 192, or a pharmaceutically acceptable salt thereof, wherein the compound is of Formula (IV):

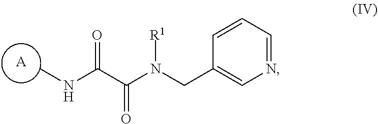

wherein the pyridinyl is substituted with 0, 1, 2 or 3 instances of R⁷.

Embodiment 265. The compound of embodiment 191 or 192, or a pharmaceutically acceptable salt thereof, wherein the compound is of Formula (A-V):

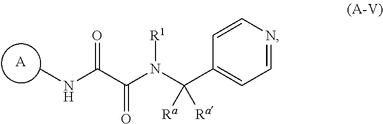

wherein the pyridinyl is substituted with 0, 1, 2 or 3 instances of R⁷.

Embodiment 266. The compound of embodiment 191 or 192, or a pharmaceutically acceptable salt thereof, wherein the compound is of Formula (V):

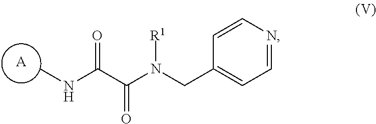

wherein the pyridinyl is substituted with 0, 1, 2 or 3 instances of R⁷.

Embodiment 267. The compound of embodiment 191 or 192, or a pharmaceutically acceptable salt thereof, wherein the compound is of Formula (A-VI):

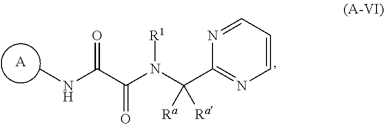

wherein the pyrimidinyl is substituted with 0, 1, 2 or 3 instances of R⁷.

Embodiment 268. The compound of embodiment 191 or 192, or a pharmaceutically acceptable salt thereof, wherein the compound is of Formula (VI):

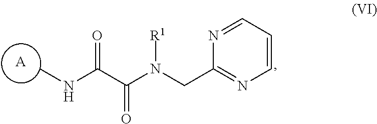

wherein the pyrimidinyl is substituted with 0, 1, 2 or 3 instances of R⁷.

Embodiment 269. The compound of any one of embodiments 1 to 268, or a pharmaceutically acceptable salt thereof, wherein each R⁷ is independently selected from the group consisting of -D, ═O, —SF₅, halo (e.g., —F, —Cl, —Br), —CN, —C₁-C₆ alkyl (e.g., -Me, -Et, —Pr, -ⁱPr, -sec-Bu, -^{t}Bu), —C₁-C₆ heteroalkyl (e.g., —CH₂OH, —CH(OH)(CH₃), —C (OH)(CH₃)₂, —CH₂NH₂), —C₁-C₆ haloalkyl (e.g., —CHF₂, —CH₂CF₃, —CF₃, —CF₂CF₃), —C₃-C₉ cycloalkyl (e.g., cyclopropyl, cyclobutyl, cyclopentyl, cyclohexyl), 3-10 membered heterocyclyl (e.g., oxetanyl, pyrolidinyl, piperidinyl, piperazinyl), phenyl, 5-10 membered heteroaryl (e.g., pyrazolyl, thiazolyl, thiophenyl, pyridinyl), cycloalkylalkyl (e.g.—CH₂-cyclopropyl), heterocyclylalkyl (e.g., —CH₂-morpholinyl), heteroarylalkyl (e.g., —CH₂-triazolyl, —CH₂-imidazolyl, —CH₂-pyrazolyl), —OR^{a7} (e.g., —OH, —OCH₃, —O-tetrahydrofuranyl, —O-tetrahydropyran-4-yl, —OCF₃, —OCHF₂), —N(R^{a7})₂, (e.g., —NH₂, —NHR^{a7}, —NHCH₃, —N(CH₃)₂), —NR^{a7}C(═O)R^{a7} (e.g., —NHC(═O)CH₃), —C(═O)N(R^{a7})₂, (e.g., —C(═O)NH₂, —C(═O)NHCH₃), OC(═O)R^{a7} (e.g., —OC(═O)CH₃), —S(═O)R^{a7} (e.g., —SO₂CH₃), —NR^{a7}S(═O)₂R^{a7} (e.g., —NHSO₂CH₃) and —S(═O)₂N(R^{a7})₂ (e.g., —SO₂NH₂, —SO₂NHCH₃), wherein each alkyl, cycloalkyl, heterocyclyl, phenyl, heteroaryl, cycloalkylalkyl, heterocyclylalkyl, arylalkyl and heteroarylalkyl is optionally substituted (e.g., substituted with 0, 1, 2 or 3 instances of -Me, —OH, —C(═O)CH₃, —NHC(═O)CH₃ or a combination thereof); and each R^{a7} is independently selected from the group consisting of H, —C₁-C₆ alkyl, (e.g., -Me,-Et, —Pr, -ⁱPr, -sec-Bu, -^{t}Bu), —C₁-C₆ haloalkyl (e.g., —CF₃, —CHF₂, —CF₂CF₃, —CH₂CF₃), —C₁-C₆ heteroalkyl substituted with 0 or 1 instances of ═O (e.g., —CH₂CH₂N(CH₃)₂, —CH₂C(═O)N(CH₃)₂, —CH(CH₃)CH₂N(CH₃)₂, —CH(CH₃) C(═O)N(CH₃)₂), C3-Co cycloalkyl and 3-10 membered heterocyclyl substituted with 0 or 1 instances of ═O, -Me or a combination thereof (e.g. tetrahydrofuran-3-yl, tetrahydropyran-4-yl, oxetan-3-yl, N—CH₃-2-oxo-pyrrolidin-3-yl).

Embodiment 270. The compound of any one of embodiments 1 to 268, or a pharmaceutically acceptable salt thereof, wherein each R⁷ is independently selected from the group consisting of -D, —O, halo (e.g., —F, —Cl, —Br), —CN, —C₁-C₆ alkyl (e.g., -Me, -Et, —Pr,-ⁱPr, -sec-Bu, -^{t}Bu), —C₁-C₆ heteroalkyl (e.g., —CH₂OH, —CH(OH)(CH₃), —C(OH)(CH₃)₂, —CH₂NH₂), —C₁-C₆ haloalkyl (e.g., —CHF₂, —CH₂CF₃, —CF₃, —CF₂CF₃), —C₃-C₉ cycloalkyl (e.g., cyclopropyl, cyclobutyl, cyclopentyl, cyclohexyl), 3-10 membered heterocyclyl (e.g., oxetanyl, pyrolidinyl, piperidinyl, piperazinyl), 5-10 membered heteroaryl (e.g., pyrazolyl, thiazolyl, thiophenyl, pyridinyl), cycloalkylalkyl (e.g.—CH₂-cyclopropyl), heterocyclylalkyl (e.g., —CH₂-morpholinyl), heteroarylalkyl (e.g., —CH₂-triazolyl, —CH₂-imidazolyl, —CH₂-pyrazolyl), —OR^{a7} (e.g., —OH, —OCH₃, —O-tetrahydrofuranyl, —O-tetrahydropyran-4-yl, —OCF₃, —OCHF₂), —N(R^{a7})₂, (e.g., —NH₂, —NHR^{a7}, —NHCH₃, —N(CH₃)₂), —NR^{a7}C(═O)R^{a7} (e.g., —NHC(═O)CH₃), —C(═O)N(R^{a7})₂, (e.g., —C(═O)NH₂, —C(═O)NHCH₃), —OC(═O)R^{a7} (e.g., —OC(═O)CH₃), —S(═O)R^{a7} (e.g., —SO₂CH₃), —NR^{a7}S(═O)₂R^{a7} (e.g., —NHSO₂CH₃) and —S(═O)₂N(R^{a7})₂ (e.g., —SO₂NH₂, —SO₂NHCH₃), wherein each alkyl, cycloalkyl, heterocyclyl, heteroaryl, cycloalkylalkyl, heterocyclylalkyl, arylalkyl and heteroarylalkyl is optionally substituted (e.g., substituted with 0, 1, 2 or 3 instances of -Me, —OH, —C(═O)CH₃, —NHC(═O)CH₃ or a combination thereof); and each R^{a7} is independently selected from the group consisting of H, —C₁-C₆ alkyl, (e.g., -Me,-Et, —Pr, -ⁱPr, -sec-Bu, -^{t}Bu), —C₁-C₆ haloalkyl (e.g., —CF₃, —CHF₂, —CF₂CF₃, —CH₂CF₃), —C₁-C₆ heteroalkyl substituted with 0 or 1 instances of ═O (e.g., —CH₂CH₂N(CH₃)₂, —CH₂C(═O)N(CH₃)₂, —CH(CH₃)CH₂N(CH₃)₂, —CH(CH₃) C(═O)N(CH₃)₂), C₃-C₉ cycloalkyl and 3-10 membered heterocyclyl substituted with 0 or 1 instances of ═O, -Me or a combination thereof (e.g. tetrahydrofuran-3-yl, tetrahydropyran-4-yl, oxetan-3-yl, N—CH₃-2-oxo-pyrrolidin-3-yl).

Embodiment 271. The compound of any one of embodiments 1 to 268, or a pharmaceutically acceptable salt thereof, wherein each R⁷ is independently selected from the group consisting of -D, halo (e.g., —F, —Cl, Br), —SF5, —CN, —C₁-C₆ alkyl (e.g., -Me, -Et, —Pr,-ⁱPr, -sec-Bu, -^{t}Bu), —C₁-C₆ heteroalkyl (e.g., —CH₂OH, —CH(OH)(CH₃), —C(OH)(CH₃)₂, —CH₂NH₂), —C₁-C₆ haloalkyl (e.g., —CHF₂, —CH₂CF₃, —CF₃), —C₃-C₉ cycloalkyl (e.g., cyclopropyl, cyclobutyl, cyclopentyl, cyclohexyl), phenyl, —OR^{a7} (e.g., —OH, —OCH₃, —O-tetrahydrofuranyl, —O-tetrahydropyran-4-yl, —OCF₃, —OCHF₂), —N(R^{a7})₂, (e.g., —NH₂, —NHR^{a7}, —NHCH₃, —N(CH₃)₂), —NR^{a7}C(═O)R^{a7} (e.g., —NHC(═O)CH₃) and —C(═O)N(R^{a7})₂, (e.g., —C(═O)NH₂, —C(═O)NHCH₃), wherein each alkyl and cycloalkyl is optionally substituted (e.g., substituted with 0, 1, 2 or 3 instances of -Me, —OH, —C(═O)CH₃, —NHC(═O)CH₃ or a combination thereof); and each R^{a7} is independently selected from the group consisting of H, —C₁-C₆ alkyl, (e.g., -Me,-Et, —Pr, -ⁱPr, -sec-Bu, -^{t}Bu) and —C₁-C₆ haloalkyl (e.g., —CF₃, —CHF₂, —CH₂CF₃).

Embodiment 272. The compound of any one of embodiments 1 to 268, or a pharmaceutically acceptable salt thereof, wherein each R⁷ is independently selected from the group consisting of -D, halo (e.g., —F, —Cl, Br), —CN, —C₁-C₆ alkyl (e.g., -Me, -Et, —Pr, -ⁱPr,-sec-Bu, -^{t}Bu), —C₁-C₆ heteroalkyl (e.g., —CH₂OH, —CH(OH)(CH₃), —C(OH)(CH₃)₂, —CH₂NH₂), —C₁-C₆ haloalkyl (e.g., —CHF₂, —CH₂CF₃, —CF₃), —C₃-C₉ cycloalkyl (e.g., cyclopropyl, cyclobutyl, cyclopentyl, cyclohexyl), —OR^{a7} (e.g., —OH, —OCH₃, —O-tetrahydrofuranyl, —O-tetrahydropyran-4-yl, —OCF₃, —OCHF₂) and —N(R^{a7})₂, (e.g., —NH₂, —NHR^{a7}, —NHCH₃, —N(CH₃)₂), wherein each alkyl and cycloalkyl is optionally substituted (e.g., substituted with 0, 1, 2 or 3 instances of -Me, —OH, —C(═O)CH₃, —NHC(═O)CH₃ or a combination thereof); and each R^{a7} is independently selected from the group consisting of H, —C₁-C₆ alkyl, (e.g., -Me,-Et, —Pr, -ⁱPr, -sec-Bu, -^{t}Bu) and —C₁-C₆ haloalkyl (e.g., —CF₃, —CHF₂, —CH₂CF₃).

Embodiment 273. The compound of any one of embodiments 1 to 268, or a pharmaceutically acceptable salt thereof, wherein each R⁷ is independently selected from the group consisting of -D, halo (e.g., —F, —Cl, Br), —SF5, —CN, —C₁-C₆ alkyl (e.g., -Me, -Et, —Pr,-ⁱPr, -sec-Bu, -^{t}Bu), —C₁-C₆ haloalkyl (e.g., —CHF₂, —CH₂CF₃, —CF₃), —N(R^{a7})₂, (e.g., —NH₂, —NHR^{a7}, —NHCH₃, —N(CH₃)₂), —NR^{a7}C(═O)R^{a7} (e.g., —NHC(═O)CH₃) and —C(═O)N(R^{a7})₂, (e.g., —C(═O)NH₂, —C(═O)NHCH₃, wherein each R^{a7} is independently selected from the group consisting of H and —C₁-C₆ alkyl, (e.g., -Me, -Et, —Pr, -ⁱPr, -sec-Bu,-^{t}Bu).

Embodiment 274. The compound of any one of embodiments 1 to 268, or a pharmaceutically acceptable salt thereof, wherein each R⁷ is independently selected from the group consisting of -D, halo (e.g., —F, —Cl, Br), —CN, —C₁-C₆ alkyl (e.g., -Me, -Et, —Pr, -ⁱPr,-sec-Bu, -^{t}Bu) and —C₁-C₆ haloalkyl (e.g., —CHF₂, —CH₂CF₃, —CF₃).

Embodiment 275. The compound of any one of embodiments 1 to 268, or a pharmaceutically acceptable salt thereof, wherein each R⁷ is independently selected from the group consisting of halo (e.g., —F, —Cl, Br), —C₁-C₆ alkyl (e.g., -Me, -Et, —Pr, -ⁱPr, -sec-Bu,-^{t}Bu), and —C₁-C₆ haloalkyl (e.g., —CHF₂, —CH₂CF₃, —CF₂CF₃, —CF₃).

Embodiment 276. The compound of any one of embodiments 1 to 268, or a pharmaceutically acceptable salt thereof, wherein each R⁷ is independently selected from the group consisting of halo (e.g., —F, —Cl, Br) and —C₁-C₆ haloalkyl (e.g., —CHF₂, —CH₂CF₃, —CF₂CF₃, —CF₃).

Embodiment 277. The compound of any one of embodiments 1 to 276, or a pharmaceutically acceptable salt thereof, wherein each R^{a7} is independently selected from the group consisting of H, -Me, -Et, —Pr, -ⁱPr, -sec-Bu, -^{t}Bu, —CF₃, —CHF₂ and —CH₂CF₃.

Embodiment 278. The compound of any one of embodiments 1 to 276, or a pharmaceutically acceptable salt thereof, wherein each R^{a7} is independently selected from the group consisting of H and -Me.

Embodiment 279. The compound of any one of embodiments 1 to 268, or a pharmaceutically acceptable salt thereof, wherein each R⁷ is independently selected from the group consisting of -D, —F, —Cl, Br, —CN, —SF5, -Me, -Et, —Pr, -ⁱPr, -sec-Bu, -^{t}Bu, —CHF₂, —CH₂CF₃, —CF₂CF₃, —CF₃, —CH₂OH, —CH(OH)(CH₃), —C(OH)(CH₃)₂, —CH₂NH₂, cyclopropyl, cyclobutyl, cyclopentyl, cyclohexyl, phenyl, pyrrolidin-1-yl, piperidin-4-yl, piperazin-4-yl, pyridin-4-yl, pyrazol-1-yl, pyrazol-3-yl, pyrazol-4-yl, pyrazol-5-yl, thiazol-2-yl, thiazol-5-yl, thiophen-2-yl, —CH₂-cyclopropyl, —CH₂-morpholin-4-yl, —CH₂-1,2,4-triazol-1-yl, —CH₂-imidazol-1-yl, —CH₂-pyrazol-1-yl, —OH, —OCH₃, —OCF₃, —OCHF₂, —O-tetrahydrofuran-3-yl, —O-tetrahydropyran-4-yl, —O—(N—CH₃-2-oxo-pyrrolidin-3-yl), —OCF₃, —OCHF₂, —NH₂, —NHCH₃, —NHCH₂CF₃, —NH-oxetan-3-yl, —NH—(N—CH₃-2-oxo-pyrrolidin-3-yl), —N(CH₃)₂, —NHC(═O)CH₃, —NHCH₂C(═O)N(CH₃)₂, —NHCH(CH₃) C(═O)N(CH₃)₂, —C(═O)NH₂, —C(═O)NHCH₃, —OC(═O)CH₃, —SO₂CH₃, —NHSO₂CH₃, —SO₂NH₂ and —SO₂NHCH₃, wherein each cyclopropyl, cyclobutyl, cyclopentyl, cyclohexyl, phenyl, pyrrolidin-1-yl, piperidin-4-yl, piperazin-4-yl, pyrazol-1-yl, pyrazol-3-yl, pyrazol-4-yl, pyrazol-5-yl, thiazol-2-yl, thiophen-2-yl, —CH₂-cyclopropyl, —CH₂-morpholin-4-yl, —CH₂-1,2,4-triazol-1-yl-CH₂-imidazol-1-yl and —CH₂-pyrazol-1-yl, can be independently substituted with 0, 1, 2 or 3 instances of -Me, —OH, —C(═O)CH₃, —NHC(═O)CH₃ or a combination thereof.

Embodiment 280. The compound of any one of embodiments 1 to 268, or a pharmaceutically acceptable salt thereof, wherein each R⁷ is independently selected from the group consisting of -D, —F, —Cl, Br, —CN, -Me, -Et, —Pr, -ⁱPr, -sec-Bu, -^{t}Bu, —CHF₂, —CH₂CF₃, —CF₂CF₃, —CF₃, —CH₂OH, —CH(OH)(CH₃), —C(OH)(CH₃)₂, —CH₂NH₂, cyclopropyl, cyclobutyl, cyclopentyl, cyclohexyl, pyrrolidin-1-yl, piperidin-4-yl, piperazin-4-yl, pyridin-4-yl, pyrazol-1-yl, pyrazol-3-yl, pyrazol-4-yl, pyrazol-5-yl, thiazol-2-yl, thiazol-5-yl, thiophen-2-yl, —CH₂-cyclopropyl, —CH₂-morpholin-4-yl, —CH₂-1,2,4-triazol-1-yl, —CH₂-imidazol-1-yl, —CH₂-pyrazol-1-yl, —OH, —OCH₃, —OCF₃, —OCHF₂, —O-tetrahydrofuran-3-yl, —O-tetrahydropyran-4-yl, —O—(N—CH₃-2-oxo-pyrrolidin-3-yl), —OCF₃, —OCHF₂, —NH₂, —NHCH₃, —NHCH₂CF₃, —NH-oxetan-3-yl, —NH—(N—CH₃-2-oxo-pyrrolidin-3-yl), —N(CH₃)₂, —NHC(═O)CH₃, —NHCH₂C(═O)N(CH₃)₂, —NHCH(CH₃) C(═O)N(CH₃)₂, —C(═O)NH₂, —C(═O)NHCH₃, —OC(═O)CH₃, —SO₂CH₃, —NHSO₂CH₃, —SO₂NH₂ and —SO₂NHCH₃, wherein each cyclopropyl, cyclobutyl, cyclopentyl, cyclohexyl, pyrrolidin-1-yl, piperidin-4-yl, piperazin-4-yl, pyrazol-1-yl, pyrazol-3-yl, pyrazol-4-yl, pyrazol-5-yl, thiazol-2-yl, thiophen-2-yl, —CH₂-cyclopropyl, —CH₂-morpholin-4-yl, —CH₂-1,2,4-triazol-1-yl-CH₂-imidazol-1-yl and —CH₂-pyrazol-1-yl, can be independently substituted with 0, 1, 2 or 3 instances of -Me, —OH, —C(═O)CH₃, —NHC(═O)CH₃ or a combination thereof.

Embodiment 281. The compound of any one of embodiments 1 to 268, or a pharmaceutically acceptable salt thereof, wherein each R⁷ is independently selected from the group consisting of -D, —F, —Cl, Br, —CN, -Me, -Et, —Pr, -ⁱPr, -sec-Bu, -^{t}Bu, —CHF₂, —CH₂CF₃, —CF₃, —CH₂OH, —CH(OH)(CH₃), —C(OH)(CH₃)₂, —CH₂NH₂, cyclopropyl, cyclobutyl, cyclopentyl, cyclohexyl, pyrrolidin-1-yl, piperidin-4-yl, piperazin-4-yl, pyridin-4-yl, pyrazol-1-yl, pyrazol-3-yl, pyrazol-4-yl, pyrazol-5-yl, thiazol-2-yl, thiazol-5-yl, thiophen-2-yl, —CH₂-cyclopropyl, —CH₂-morpholin-4-yl, —CH₂-1,2,4-triazol-1-yl, —CH₂-imidazol-1-yl, —CH₂-pyrazol-1-yl, —OH, —OCH₃, —OCF₃, —OCHF₂, —O-tetrahydrofuran-3-yl, —O-tetrahydropyran-4-yl, —O—(N—CH₃-2-oxo-pyrrolidin-3-yl), —OCF₃, —OCHF₂, —NH₂, —NHCH₃, —NHCH₂CF₃, —NH-oxetan-3-yl, —NH—(N—CH₃-2-oxo-pyrrolidin-3-yl), —N(CH₃)₂, —NHC(═O)CH₃, —NHCH₂C(═O)N(CH₃)₂, —NHCH(CH₃) C(═O)N(CH₃)₂, —C(═O)NH₂, —C(═O)NHCH₃, —OC(═O)CH₃, —SO₂CH₃, —NHSO₂CH₃, —SO₂NH₂ and —SO₂NHCH₃, wherein each cyclopropyl, cyclobutyl, cyclopentyl, cyclohexyl, pyrrolidin-1-yl, piperidin-4-yl, piperazin-4-yl, pyrazol-1-yl, pyrazol-3-yl, pyrazol-4-yl, pyrazol-5-yl, thiazol-2-yl, thiophen-2-yl, —CH₂-cyclopropyl, —CH₂-morpholin-4-yl, —CH₂-1,2,4-triazol-1-yl-CH₂-imidazol-1-yl and —CH₂-pyrazol-1-yl, can be independently substituted with 0, 1, 2 or 3 instances of -Me, —OH, —C(═O)CH₃, —NHC(═O)CH₃ or a combination thereof.

Embodiment 282. The compound of any one of embodiments 1 to 268, or a pharmaceutically acceptable salt thereof wherein each R⁷ is independently selected from the group consisting of -D, —O, —F, —Cl, -Me, -ⁱPr, —CHF₂, —CF₂CF₃, —CF₃, —CN, —SF5, cyclopropyl, piperidin-4-yl, piperazin-4-yl, phenyl, pyrazol-1-yl, pyrazol-3-yl, pyrazol-4-yl, pyrazol-5-yl, —OH, —OCH₃, —OCF₃, —OCHF₂, —NH₂, —NHCH₃, —N(CH₃)₂, —NHC (O)CH₃, —CONH₂, wherein each cyclopropyl, phenyl, piperazin-4-yl, pyrazol-1-yl, pyrazol-3-yl, pyrazol-4-yl and pyrazol-5-yl, can be independently substituted with 0, 1, 2 or 3 instances of-Me, —OH, —C(═O)CH₃, —NHC(═O)CH₃ or a combination thereof.

Embodiment 283. The compound of any one of embodiments 1 to 268, or a pharmaceutically acceptable salt thereof wherein each R⁷ is independently selected from the group consisting of -D, ═O, —F, —Cl, -Me, -ⁱPr, —CHF₂, —CF₃, cyclopropyl, piperidin-4-yl, piperazin-4-yl, pyrazol-1-yl, pyrazol-3-yl, pyrazol-4-yl, pyrazol-5-yl, —OH, —OCH₃, —OCF₃, —OCHF₂, wherein each cyclopropyl, piperazin-4-yl, pyrazol-1-yl, pyrazol-3-yl, pyrazol-4-yl and pyrazol-5-yl, can be independently substituted with 0, 1, 2 or 3 instances of -Me, —OH, —C(═O)CH₃, —NHC (═O)CH₃ or a combination thereof.

Embodiment 284. The compound of any one of embodiments 1 to 268, or a pharmaceutically acceptable salt thereof wherein each R⁷ is independently selected from the group consisting of —F, —Cl, -Me, —Pr, —CF₂CF₃, —CF₃, —CN, —SF5, phenyl, —NH₂, —NHCH₃, —N(CH₃)₂, —NHC (O)CH₃ and —CONH₂.

Embodiment 285. The compound of any one of embodiments 1 to 268, or a pharmaceutically acceptable salt thereof wherein each R⁷ is independently selected from the group consisting of —F, —Cl, -Me, —CF₂CF₃ and —CF₃.

Embodiment 286. The compound of any one of embodiments 1 to 268, or a pharmaceutically acceptable salt thereof wherein each R⁷ is independently selected from the group consisting of —F, —Cl, and —CF₃.

Embodiment 287. The compound of any one of embodiments 1 to 268, or a pharmaceutically acceptable salt thereof wherein each R⁷ is independently selected from the group consisting of —F and —CF₃.

Embodiment 288. The compound of any one of embodiments 1 to 268, or a pharmaceutically acceptable salt thereof wherein each R⁷ is independently —Cl.

Embodiment 289. The compound of any one of embodiments 1 to 268, or a pharmaceutically acceptable salt thereof wherein each R⁷ is independently —CH₃.

Embodiment 290. The compound of any one of embodiments 1 to 268, or a pharmaceutically acceptable salt thereof wherein each R⁷ is independently-CF₂CF₃.

Embodiment 291. The compound of any one of embodiments 1 to 268, or a pharmaceutically acceptable salt thereof wherein each R⁷ is independently —CF₃.

Embodiment 292. The compound of any one of embodiments 1 to 268, or a pharmaceutically acceptable salt thereof, wherein each R⁷ is independently-F.

Embodiment 293. The compound of any one of embodiments 1 to 190 or a pharmaceutically acceptable salt thereof wherein Ring B is selected from:

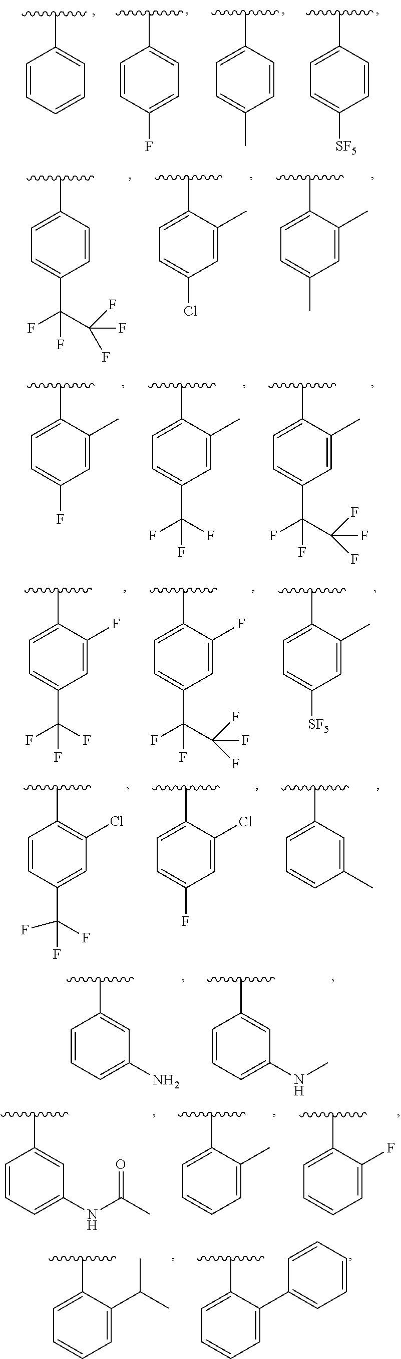

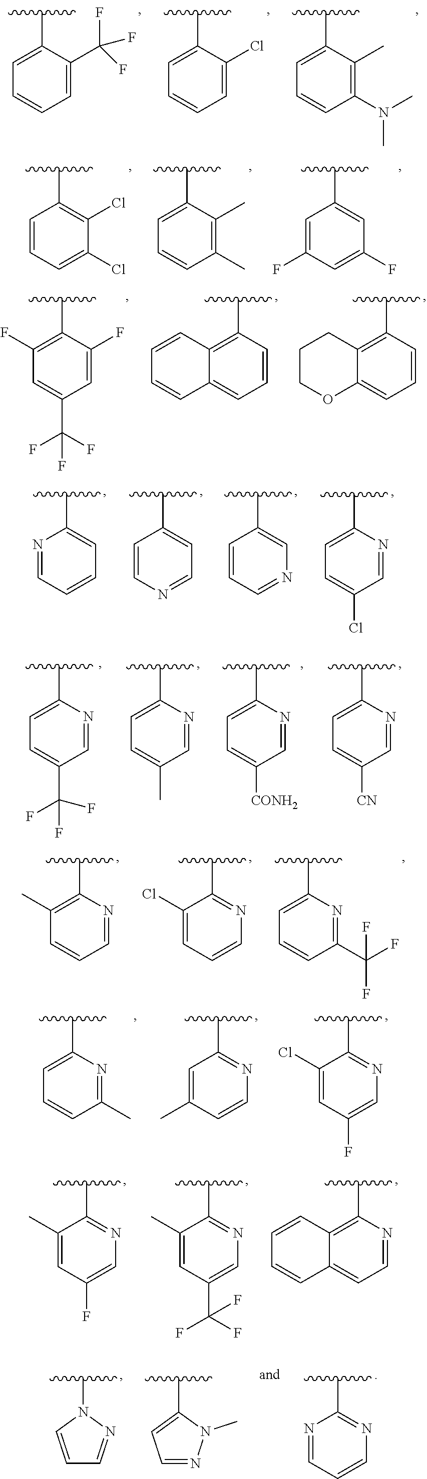

Embodiment 294. The compound of any one of embodiments 1 to 293, or a pharmaceutically acceptable salt thereof wherein each R¹ is independently selected from the group consisting of —C₁-C₆ alkyl, —C₂-C₆ heteroalkyl, —C₂-C₆ haloalkyl, —C₃-C₁₀ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each substituted at any available position with 0, 1, 2 or 3 instances of R⁸ wherein each R⁸ is independently selected from the group consisting of halo, —O, —CN, —OH, —NH₂, —C₁-C₆ alkyl, —C₁-C₆ haloalkyl, —O(C₁-C₆ alkyl), —O(C₁-C₆ haloalkyl), —NH(C₁-C₆ alkyl), —NH(C₁-C₆ haloalkyl), —N(C₁-C₆ alkyl)₂, —N(C₁-C₆ haloalkyl)₂, —C(O)NH₂, —NHC (O)(C₁-C₆ alkyl), C₃-C₉ cycloalkyl and C1-C6 heteroalkyl.

Embodiment 295. The compound of any one of embodiments 1 to 293, or a pharmaceutically acceptable salt thereof wherein each R¹ is independently selected from the group consisting of —C₂-C₆ alkyl, —C₂-C₆ heteroalkyl, —C₂-C₆ haloalkyl, —C₃-C₉ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each substituted at any available position with 0, 1, 2 or 3 instances of R⁸ wherein each R⁸ is independently selected from the group consisting of halo, —O, —CN, —OH, —NH₂, —C₁-C₆ alkyl, —C₁-C₆ haloalkyl, —O(C₁-C₆ alkyl), —O(C₁-C₆ haloalkyl), —NH(C₁-C₆ alkyl), —NH(C₁-C₆ haloalkyl), —N(C₁-C₆ alkyl)₂, —N(C₁-C₆ haloalkyl)₂, C₃-C₉ cycloalkyl and C₁-C₆ heteroalkyl.

Embodiment 296. The compound of any one of embodiments 1 to 293, or a pharmaceutically acceptable salt thereof wherein each R¹ is independently selected from the group consisting of —C₂-C₆ alkyl, —C₂-C₆ heteroalkyl, —C₂-C₆ haloalkyl, —C₃-C₉ carbocyclyl, C₆-C₁₀ aryl, 5-10 membered heteroaryl, 3-10 membered heterocyclyl, heterocyclylalkyl, heteroarylalkyl, arylalkyl and cycloalkylalkyl, each substituted at any available position with 0, 1, 2 or 3 instances of R⁸ wherein each R⁸ is independently selected from the group consisting of halo, —O, —CN, —OH, —NH₂, —C₁-C₆ alkyl, —O(C₁-C₆ alkyl), —O(C₁-C₆ haloalkyl), —NH(C₁-C₆ alkyl), —NH(C₁-C₆ haloalkyl), —N(C₁-C₆ alkyl)₂, —N(C₁-C₆ haloalkyl)₂, C₃-C₉ cycloalkyl and C₁-C₆ heteroalkyl.

Embodiment 297. The compound of any one of embodiments 1 to 293, or a pharmaceutically acceptable salt thereof wherein each R¹ is independently selected from the group consisting of —C₁-C₆ alkyl (e.g., -Me, -Et, —Pr, -ⁱPr, -sec-Bu, -^{t}Bu, —CH(CH₃)CH(CH₃)₂, —CH₂CH(CH₃)₂, —CH₂CH(CH₃)CH₂CH₃), —C₂-C₆ heteroalkyl (e.g., —CH₂CH₂OCH₃), —C₂-C₆ haloalkyl (e.g., —CH₂CH₂CF₃), —C₃-C₁₀ carbocyclyl (e.g., cyclopropyl, cyclobutyl, cyclopentyl, cyclohexyl, 2,3-dihydro-1H-indenyl, 1,2,3,4 tetrahydronaphthalenyl), 3-10 membered heterocyclyl (e.g., chromanyl), heteroarylalkyl (e.g., —CH₂-pyridinyl, —CH(CH₃)-pyridinyl-CH₂-pyrimidinyl, —CH(CH₃)-pyrimidinyl, —CH₂-pyrazolyl), arylalkyl (e.g., benzyl, —CH(CH₃)phenyl, —CH₂-naphthalenyl, —CH₂-chromanyl, —CH₂CH₂-phenyl), heterocyclylalkyl (e.g., CH₂-tetrahydropyranyl) and cycloalkylalkyl (e.g., —CH₂-cyclopropyl, —CH₂-cyclobutyl, —CH₂-cyclopentyl, —CH₂-cyclohexyl, —CH(CH₃)cyclopropyl, —CH₂CH₂-cyclopropyl), each substituted at any available position with 0, 1, 2 or 3 instances of R⁸ wherein each R⁸ is independently selected from the group consisting of halo (e.g., —F, —Cl), ═O, —CN, —OH, —NH₂, —C₁-C₆ alkyl (e.g., -Me, -Et-, —Pr, -ⁱPr, -sec-Bu, -^{t}Bu), —C₁-C₆ haloalkyl (e.g., —CF₃), —O(C₁-C₆ alkyl)(e.g., —OCH₃, —OCH₂CH₃, —OCH(CH₃)₂), —O(C₁-C₆ haloalkyl)(e.g., —OCF₃, —OCHF₂, —OCH₂CF₃), C₃-C₉ cycloalkyl (e.g., cyclopropyl, cyclobutyl, cyclopentyl, cyclohexyl) and C₁-C₆ heteroalkyl (e.g., —CH₂OCH₃, —CH₂CH₂OCH₃, —CH₂NHCH₃, —CH₂CH₂NHCH₃, —CH₂N(CH₃)₂, —CH₂CH₂N(CH₃)₂).

Embodiment 298. The compound of any one of embodiments 1 to 293, or a pharmaceutically acceptable salt thereof wherein each R¹ is independently selected from the group consisting of —C₂-C₆ alkyl (e.g., -Me, -Et, —Pr, —Pr, -sec-Bu, -^{t}Bu, —CH(CH₃)CH(CH₃)₂, —CH₂CH(CH₃)₂, —CH₂CH(CH₃)CH₂CH₃), —C₂-C₆ heteroalkyl (e.g., —CH₂CH₂OCH₃), —C₃-C₉ carbocyclyl (e.g., cyclopropyl, cyclobutyl, cyclopentyl, cyclohexyl, 2,3-dihydro-1H-indenyl, 1,2,3,4 tetrahydronaphthalenyl), heteroarylalkyl (e.g., —CH₂-pyridinyl, —CH(CH₃)-pyridinyl-CH₂-pyrimidinyl, —CH(CH₃)-pyrimidinyl), arylalkyl (e.g., benzyl, —CH(CH₃)phenyl, —CH₂CH₂-phenyl), and cycloalkylalkyl (e.g., —CH₂-cyclopropyl, —CH₂-cyclobutyl, —CH₂-cyclopentyl, —CH₂-cyclohexyl, —CH(CH₃)cyclopropyl, —CH₂CH₂-cyclopropyl), each substituted at any available position with 0, 1, 2 or 3 instances of R⁸ wherein each R⁸ is independently selected from the group consisting of halo (e.g., —F, —Cl), —O, —CN, —OH, —NH₂, —C₁-C₆ alkyl (e.g., -Me, -Et-, —Pr, -ⁱPr, -sec-Bu, -^{t}Bu), —C₁-C₆ haloalkyl (e.g., —CF₃), —O(C₁-C₆ alkyl)(e.g., —OCH₃, —OCH₂CH₃, —OCH(CH₃)₂), —O(C₁-C₆ haloalkyl)(e.g., —OCF₃, —OCHF₂, —OCH₂CF₃), C₃-C₉ cycloalkyl (e.g., cyclopropyl, cyclobutyl, cyclopentyl, cyclohexyl) and C₁-C₆ heteroalkyl (e.g., —CH₂OCH₃, —CH₂CH₂OCH₃, —CH₂NHCH₃, —CH₂CH₂NHCH₃, —CH₂N(CH₃)₂, —CH₂CH₂N(CH₃)₂).

Embodiment 299. The compound of any one of embodiments 1 to 293, or a pharmaceutically acceptable salt thereof wherein each R¹ is independently selected from the group consisting of —C₂-C₆ alkyl (e.g., -Et, —Pr, —Pr, -sec-Bu, -^{t}Bu, —CH(CH₃)CH(CH₃)₂), —C₂-C₆ heteroalkyl (e.g., —CH₂CH₂OCH₃), —C₃-C₉ carbocyclyl (e.g., cyclopropyl, cyclobutyl, cyclopentyl, cyclohexyl, 2,3-dihydro-1H-indenyl, 1,2,3,4 tetrahydronaphthalenyl), heteroarylalkyl (e.g., —CH₂-pyridinyl, —CH(CH₃)-pyridinyl-CH₂-pyrimidinyl, —CH(CH₃)-pyrimidinyl), arylalkyl (e.g., benzyl, —CH(CH₃)phenyl, —CH₂CH₂-phenyl), and cycloalkylalkyl (e.g., —CH₂-cyclopropyl, —CH₂-cyclobutyl, —CH₂-cyclopentyl, —CH₂-cyclohexyl, —CH(CH₃)cyclopropyl, —CH₂CH₂-cyclopropyl), each substituted at any available position with 0, 1, 2 or 3 instances of R⁸ wherein each R⁸ is independently selected from the group consisting of halo (e.g., —F, —Cl), ═O, —CN, —OH, —NH₂, —C₁-C₆ alkyl (e.g., -Me, -Et-, —Pr, -ⁱPr, -sec-Bu, -^{t}Bu), —O(C₁-C₆ alkyl)(e.g., —OCH₃, —OCH₂CH₃, —OCH(CH₃)₂), —O(C1-C6 haloalkyl)(e.g., —OCF₃, —OCHF₂, —OCH₂CF₃), C₃-C₉ cycloalkyl (e.g., cyclopropyl, cyclobutyl, cyclopentyl, cyclohexyl) and C₁-C₆ heteroalkyl (e.g., —CH₂OCH₃, —CH₂CH₂OCH₃, —CH₂NHCH₃, —CH₂CH₂NHCH₃, —CH₂N(CH₃)₂, —CH₂CH₂N(CH₃)₂).

Embodiment 300. The compound of any one of embodiments 1 to 293, or a pharmaceutically acceptable salt thereof wherein each R¹ is independently selected from the group consisting of —C₁-C₆ alkyl (e.g., -Me, -Et, —Pr, —Pr, -sec-Bu, -^{t}Bu, —CH(CH₃)CH(CH₃)₂, —CH₂CH(CH₃)₂, —CH₂CH(CH₃)CH₂CH₃), —C₂-C₆ heteroalkyl (e.g., —CH₂CH₂OCH₃) and arylalkyl (e.g., benzyl, —CH(CH₃)phenyl, —CH₂-naphthalenyl, —CH₂-chromanyl), each substituted at any available position with 0, 1 or 2 instances of R⁸ wherein each R8 is independently selected from the group consisting of halo (e.g., —F, —Cl) and -C1-C6 alkyl (e.g., -Me, -Et-, —Pr, -ⁱPr, -sec-Bu, -^{t}Bu).

Embodiment 301. The compound of any one of embodiments 1 to 293, or a pharmaceutically acceptable salt thereof wherein each R¹ is independently selected from the group consisting of —C₁-C₆ alkyl (e.g., -Me, -Et, —Pr, -ⁱPr, -sec-Bu, -^{t}Bu, —CH(CH₃)CH(CH₃)₂, —CH₂CH(CH₃)₂, —CH₂CH(CH₃)CH₂CH₃), —C₂-C₆ heteroalkyl (e.g., —CH₂CH₂OCH₃) and arylalkyl (e.g., benzyl, —CH(CH₃)phenyl, —CH₂-naphthalenyl, —CH₂-chromanyl) wherein the alkyl and the arylalkyl are not further substituted.

Embodiment 302. The compound of any one of embodiments 1 to 293, or a pharmaceutically acceptable salt thereof wherein each R¹ is independently selected from the group consisting of -Me, -Et, —Pr, -ⁱPr, -sec-Bu, -^{t}Bu, —CH(CH₃)CH(CH₃)₂, —CH₂CH(CH₃)₂, —CH₂CH(CH₃)CH₂CH₃, —CH₂CH₂OCH₃, —CH₂CH₂CF₃, cyclopropyl, cyclobutyl, cyclopentyl, cyclohexyl, 2,3-dihydro-1H-indenyl, 1,2,3,4 tetrahydronaphthalenyl, chromanyl, —CH₂-pyridinyl, —CH(CH₃)-pyridinyl-CH₂-pyrimidinyl, —CH(CH₃)-pyrimidinyl, —CH₂-pyrazolyl, benzyl, —CH(CH₃)phenyl, —CH₂CH₂-phenyl, CH₂-naphthyl, —CH₂-chromanyl, —CH₂-tetrahydropyranyl, —CH₂-cyclopropyl, —CH₂-cyclobutyl, —CH₂-cyclopentyl, —CH₂-cyclohexyl, —CH(CH₃)cyclopropyl, and —CH₂CH₂-cyclopropyl, each substituted at any available position with 0, 1, 2 or 3 instances of R⁸ wherein each R⁸ is independently selected from the group consisting of —F, —Cl, —O, —CN, —OH, —NH₂, —NHCH₃, —N(CH₃)₂, —NHC (O)CH₃, —CONH₂, -Me, -Et-, —Pr, -ⁱPr, -sec-Bu, -^{t}Bu, —CF₃, —OCH₃, —OCH₂CH₃, —OCH(CH₃)₂, —OCF₃, —OCHF₂, —OCH₂CF₃, cyclopropyl, cyclobutyl, cyclopentyl, cyclohexyl, —CH₂OCH₃, —CH₂CH₂OCH₃, —CH₂NHCH₃, —CH₂CH₂NHCH₃, —CH₂N(CH₃)₂ and —CH₂CH₂N(CH₃)₂.

Embodiment 303. The compound of any one of embodiments 1 to 293, or a pharmaceutically acceptable salt thereof wherein each R¹ is independently selected from the group consisting of -Me, -Et, —Pr, -ⁱPr, -sec-Bu, -^{t}Bu, —CH(CH₃)CH(CH₃)₂, —CH₂CH(CH₃)₂, —CH₂CH(CH₃)CH₂CH₃, —CH₂CH₂OCH₃, cyclopropyl, cyclobutyl, cyclopentyl, cyclohexyl, 2,3-dihydro-1H-indenyl, 1,2,3,4 tetrahydronaphthalenyl, —CH₂-pyridinyl, —CH(CH₃)-pyridinyl-CH₂-pyrimidinyl, —CH(CH₃)-pyrimidinyl, benzyl, —CH(CH₃)phenyl, —CH₂CH₂-phenyl, —CH₂-cyclopropyl, —CH₂-cyclobutyl, —CH₂-cyclopentyl, —CH₂-cyclohexyl, —CH(CH₃)cyclopropyl, and —CH₂CH₂-cyclopropyl, each substituted at any available position with 0, 1, 2 or 3 instances of R⁸ wherein each R⁸ is independently selected from the group consisting of —F, —Cl, —O, —CN, —OH, —NH₂, -Me, -Et-, —Pr, —Pr, -sec-Bu, -‘Bu, —OCH₃, —OCH₂CH₃, —OCH(CH₃)₂, —OCF₃, —OCHF₂, —OCH₂CF₃, cyclopropyl, cyclobutyl, cyclopentyl, cyclohexyl, —CH₂OCH₃, —CH₂CH₂OCH₃, —CH₂NHCH₃, —CH₂CH₂NHCH₃, —CH₂N(CH₃)₂ and —CH₂CH₂N(CH₃)₂.

Embodiment 304. The compound of any one of embodiments 1 to 293, or a pharmaceutically acceptable salt thereof wherein each R¹ is independently selected from the group consisting of -Et, —Pr, -ⁱPr, -sec-Bu, -Bu, —CH(CH₃)CH(CH₃)₂, —CH₂CH₂OCH₃, cyclopropyl, cyclobutyl, cyclopentyl, cyclohexyl, 2,3-dihydro-1H-indenyl, 1,2,3,4 tetrahydronaphthalenyl, —CH₂-pyridinyl, —CH(CH₃)-pyridinyl-CH₂-pyrimidinyl, —CH(CH₃)-pyrimidinyl, benzyl, —CH(CH₃)phenyl, —CH₂CH₂-phenyl, —CH₂-cyclopropyl, —CH₂-cyclobutyl, —CH₂-cyclopentyl, —CH₂-cyclohexyl, —CH(CH₃)cyclopropyl, and —CH₂CH₂-cyclopropyl, each substituted at any available position with 0, 1, 2 or 3 instances of R⁸ wherein each R⁸ is independently selected from the group consisting of —F, —C1, =0, —CN, —OH, —NH₂, -Me, -Et-, —Pr, -ⁱPr, -sec-Bu, -^{t}Bu, —OCH₃, —OCH₂CH₃, —OCH(CH₃)₂, —OCF₃, —OCHF₂, —OCH₂CF₃, cyclopropyl, cyclobutyl, cyclopentyl, cyclohexyl, —CH₂OCH₃, —CH₂CH₂OCH₃, —CH₂NHCH₃, —CH₂CH₂NHCH₃, —CH₂N(CH₃)₂ and —CH₂CH₂N(CH₃)₂.

Embodiment 305. The compound of any one of embodiments 1 to 293, or a pharmaceutically acceptable salt thereof wherein each R¹ is independently selected from the group consisting of -Me, -Et, —Pr, —CH(CH₃)CH(CH₃)₂, —CH₂CH(CH₃)₂, —CH₂CH(CH₃)CH₂CH₃, —CH₂CH₂OCH₃, —CH₂CH₂CF₃, cyclopropyl, cyclobutyl, cyclopentyl, 2,3-dihydro-1H-indenyl, 1,2,3,4 tetrahydronaphthalenyl, chromanyl, —CH₂-cyclopropyl, —CH₂-cyclohexyl, —CH₂CH₂-cyclopropyl, —CH₂-tetrahydropyranyl, —CH₂-pyridinyl, -CH₂-pyrimidinyl, —CH₂-pyrazolyl, -benzyl CH₂-chromanyl, CH₂-naphthyl, and —CH₂-cyclopropyl, each substituted at any available position with 0, 1 or 2 instances of R⁸ wherein each R8 is independently selected from the group consisting of —F, —Cl, -Me, —NH₂, —NHCH₃, —N(CH₃)₂, —NHC (O)CH₃, —C(O)NH₂, —CF₃, —OCHF₂, -cyclopropyl, and —CH₂OCH₃.

Embodiment 306. The compound of any one of embodiments 1 to 293, or a pharmaceutically acceptable salt thereof wherein each R¹ is independently selected from the group consisting of -Me, -Et, —Pr, —Pr, —CH(CH₃)CH(CH₃)₂, —CH₂CH(CH₃)₂, —CH₂CH(CH₃)CH₂CH₃, —CH₂CH₂OCH₃, cyclopentyl, 2,3-dihydro-1H-inden-1-yl, 1,2,3,4 tetrahydronaphthalen-1-yl, —CH₂-pyrimidinyl, —CH(CH₃)-pyrimidinyl, benzyl and —CH₂-cyclopropyl, each substituted at any available position with 0 or 1 instances of R⁸ wherein each R8 is independently selected from the group consisting of —F, -Me, —CF₃, —OCHF₂, -cyclopropyl, and —CH₂OCH₃.

Embodiment 307. The compound of any one of embodiments 1 to 293, or a pharmaceutically acceptable salt thereof wherein each R¹ is independently selected from the group consisting of -Et, —Pr, —CH(CH₃)CH(CH₃)₂, —CH₂CH₂OCH₃, cyclopentyl, 2,3-dihydro-1H-inden-1-yl, 1,2,3,4 tetrahydronaphthalen-1-yl, —CH₂-pyrimidinyl, —CH(CH₃)-pyrimidinyl, benzyl and —CH₂-cyclopropyl, each substituted at any available position with 0 or 1 instances of R⁸ wherein each R⁸ is independently selected from the group consisting of —F,-Me, —OCHF₂, -cyclopropyl, and —CH₂OCH₃.

Embodiment 308. The compound of any one of embodiments 1 to 293, or a pharmaceutically acceptable salt thereof wherein each R¹ is independently selected from the group consisting of -Me, -Et, —Pr, —CH(CH₃)CH(CH₃)₂, —CH₂CH(CH₃)₂, —CH₂CH(CH₃)CH₂CH₃, —CH₂CH₂CF₃, —CH₂CH₂OCH₃,

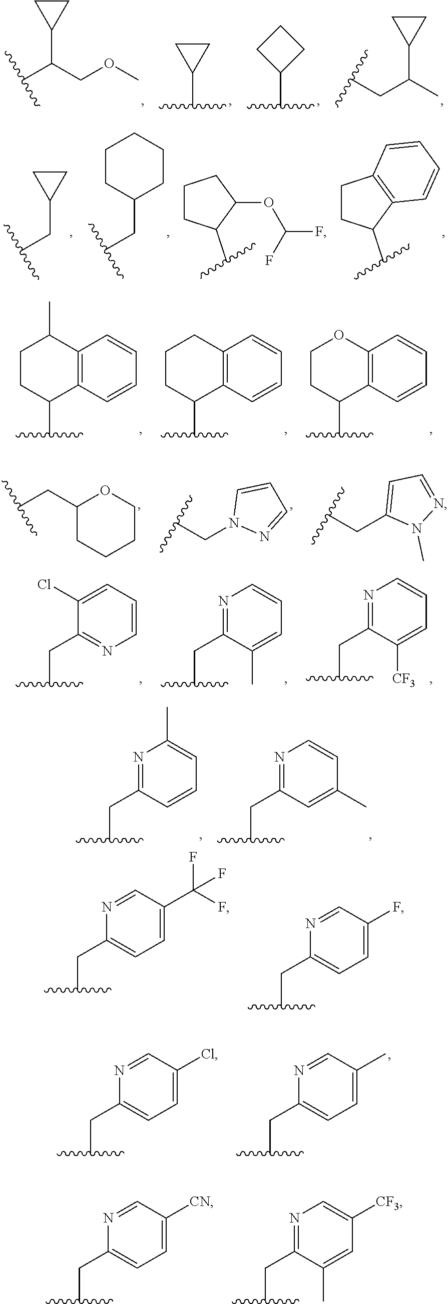

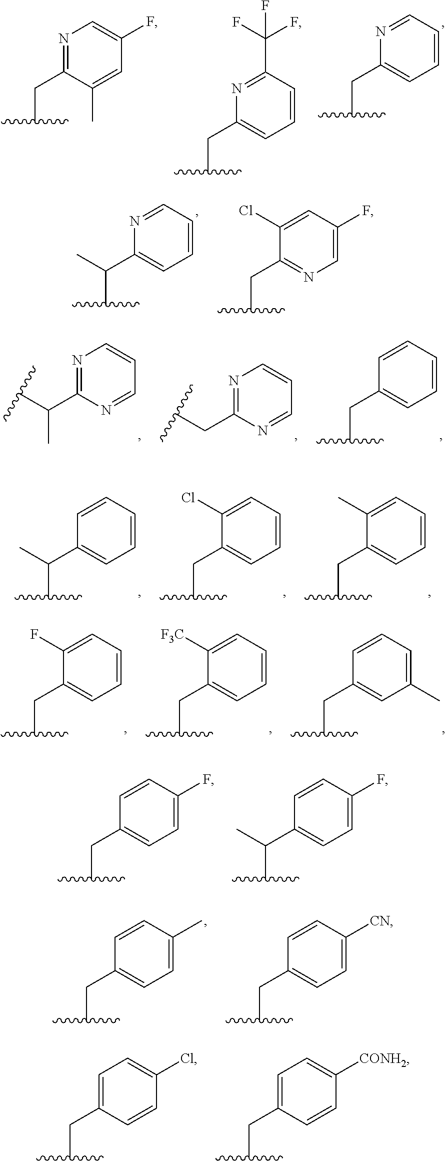

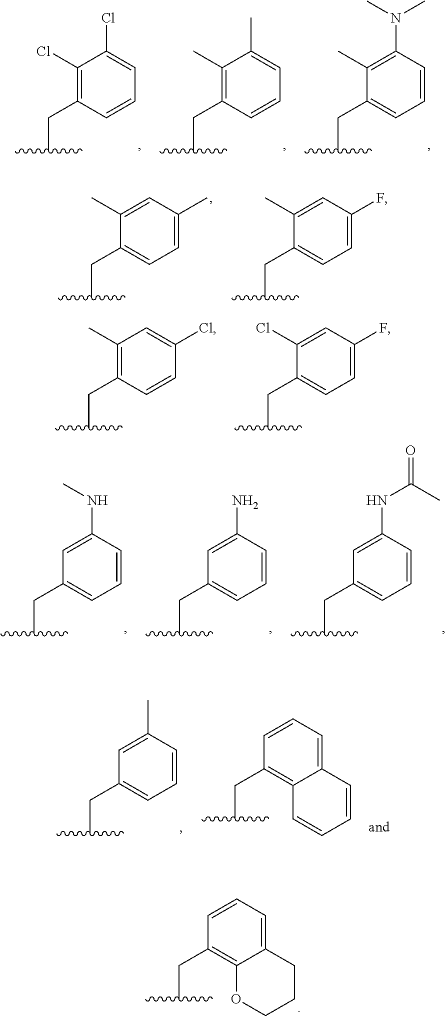

Embodiment 309. The compound of any one of embodiments 1 to 293, or a pharmaceutically acceptable salt thereof wherein each R¹ is independently selected from the group consisting of -Me, -Et, —Pr, —CH(CH₃)CH(CH₃)₂, —CH₂CH(CH₃)₂, —CH₂CH(CH₃)CH₂CH₃, —CH₂CH₂OCH₃,

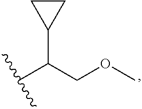 [SMILES: *C(COC)C1CC1]

cyclobutyl,

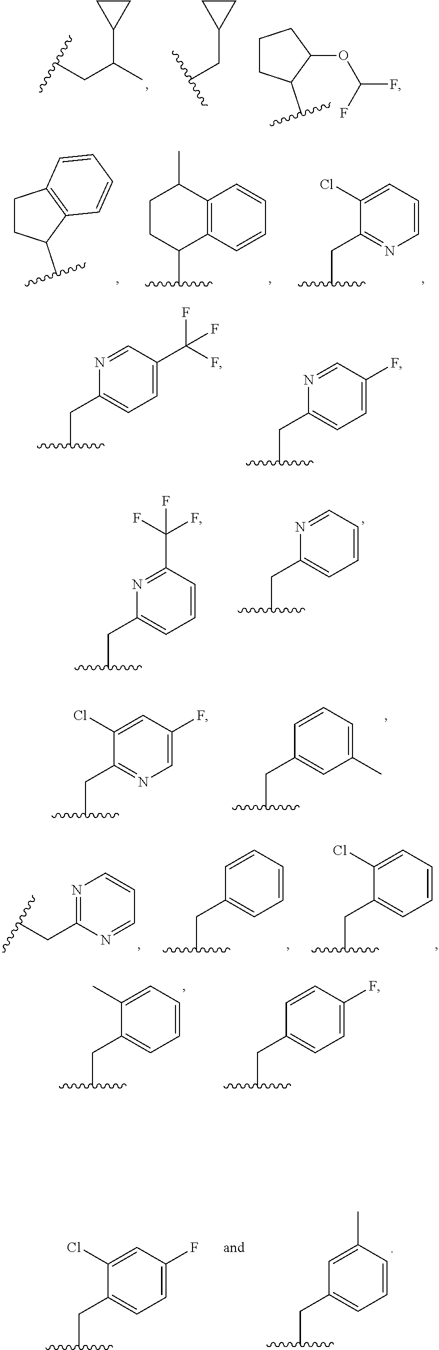

Embodiment 310. The compound of any one of embodiments 1 to 293, or a pharmaceutically acceptable salt thereof wherein each R¹ is independently selected from the group consisting of -Me, -Et, —Pr, —CH(CH₃)CH(CH₃)₂, —CH₂CH(CH₃)₂, —CH₂CH(CH₃)CH₂CH₃, —CH₂CH₂OCH₃,

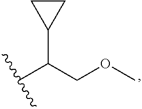 [SMILES: *C(COC)C1CC1]

cyclobutyl,

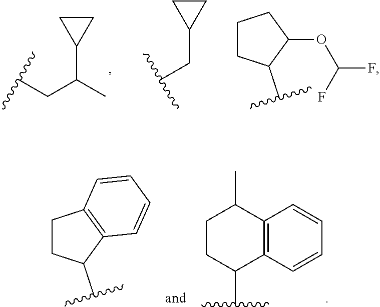

Embodiment 311. The compound of any one of embodiments 1 to 293, or a pharmaceutically acceptable salt thereof wherein each R¹ is independently selected from the group consisting of -Me, -Et, —Pr, —CH(CH₃)CH(CH₃)₂, —CH₂CH(CH₃)₂, —CH₂CH(CH₃)CH₂CH₃, —CH₂CH₂OCH₃, —CH₂CH₂CF₃, cyclopropyl, cyclobutyl, cyclopentyl, 2,3-dihydro-1H-indenyl, 1,2,3,4 tetrahydronaphthalenyl, chromanyl, —CH₂-cyclopropyl, -—CH₂-cyclohexyl, —CH(CH₃)cyclopropyl, and —CH₂CH₂-cyclopropyl, each substituted at any available position with 0 or 1 instances of R⁸ wherein each R⁸ is independently selected from the group consisting of -Me and —OCHF₂.

Embodiment 312. The compound of any one of embodiments 1 to 293, or a pharmaceutically acceptable salt thereof wherein each R¹ is independently selected from the group consisting of -Me, -Et, —Pr, —CH(CH₃)CH(CH₃)₂, —CH₂CH(CH₃)₂, —CH₂CH(CH₃)CH₂CH₃, —CH₂CH₂CF₃, —CH₂CH₂OCH₃.

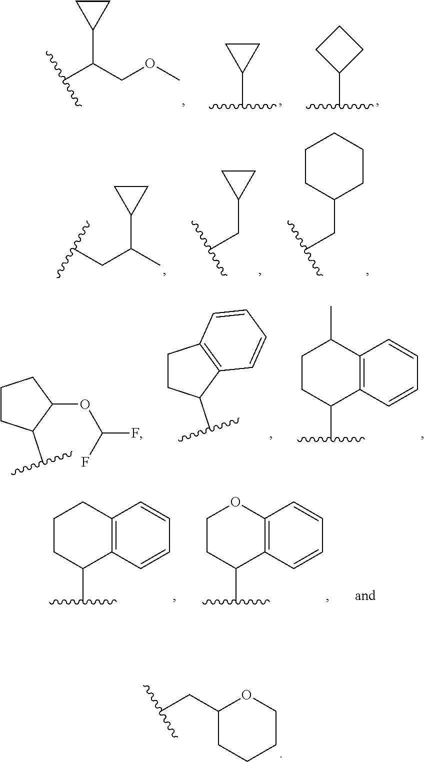

Embodiment 313. The compound of any one of embodiments 1 to 293, or a pharmaceutically acceptable salt thereof wherein each R¹ is independently selected from the group consisting of -Me, -Et, -ⁱPr, —CH(CH₃)CH(CH₃)₂, —CH₂CH(CH₃)₂ and —CH₂CH(CH₃)CH₂CH₃.

Embodiment 314. The compound of any one of embodiments 1 to 293, or a pharmaceutically acceptable salt thereof wherein each R¹ is independently selected from the group consisting of -Me, -Et, benzyl, —CH₂-pyridinyl and CH₂-pyrimidinyl, wherein the benzyl, —CH₂-pyridinyl and CH₂-pyrimidinyl are substituted at any available positions with 0, 1 or 2 substituents independently selected from -Me, —F, —Cl and —CF₃.

Embodiment 315. The compound of any one of embodiments 1 to 293, or a pharmaceutically acceptable salt thereof wherein each R¹ is independently selected from the group consisting of benzyl, —CH₂-pyridinyl and CH₂-pyrimidinyl, wherein the benzyl, —CH₂-pyridinyl and CH₂-pyrimidinyl are substituted at any available positions with 0, 1 or 2 substituents independently selected from -Me, —F, —Cl and —CF₃.

Embodiment 316. The compound of any one of embodiments 1 to 293, or a pharmaceutically acceptable salt thereof wherein each R¹ is independently selected from the group consisting of -Me, -Et and benzyl wherein the benzyl is substituted at any available positions with 0, 1 or 2 substituents independently selected from Me, —F, —Cl and —CF₃.

Embodiment 317. The compound of any one of embodiments 1 to 293, or a pharmaceutically acceptable salt thereof wherein each R¹ is independently selected from the group consisting of -Me, -Et, —CH₂-phenyl and —CH(CH₃)phenyl, wherein the phenyl is substituted at any available positions with 0, 1 or 2 substituents independently selected from Me, —F, —Cl and —CF₃.

Embodiment 318. The compound of any one of embodiments 1 to 293, or a pharmaceutically acceptable salt thereof wherein each R¹ is independently selected from the group consisting of -Me and -Et.

Embodiment 319. The compound of any one of embodiments 1 to 293, or a pharmaceutically acceptable salt thereof wherein R¹ is -Me.

Embodiment 320. The compound of any one of embodiments 1 to 293, or a pharmaceutically acceptable salt thereof wherein R¹ is -Et.

Embodiment 321. The compound of any one of embodiments 1 to 293, or a pharmaceutically acceptable salt thereof wherein R¹ is benzyl substituted at any available positions with 0, 1 or 2 substituents independently selected from Me, —F, —Cl and —CF ₃.

Embodiment 322. The compound of any one of embodiments 1 to 293, or a pharmaceutically acceptable salt thereof wherein each R¹ is —CH₂-pyridinyl substituted at any available positions with 0, 1 or 2 substituents independently selected from -Me, —F, —Cl and —CF₃.

Embodiment 323. The compound of any one of embodiments 1 to 293, or a pharmaceutically acceptable salt thereof wherein each R¹ is CH₂-pyrimidinyl substituted at any available positions with 0, 1 or 2 substituents independently selected from -Me, —F, —C1 and —CF₃.

Embodiment 324. The compound of any one of embodiments 1 to 323 wherein the compound is selected from the group consisting of Table 1, or a pharmaceutically acceptable salt thereof.

Embodiment 325.A pharmaceutical composition comprising a compound of any one of embodiments 1 to 324, or a pharmaceutically acceptable salt thereof, and a pharmaceutically acceptable carrier.

Embodiment 326. The pharmaceutical composition of embodiment 325, further comprising a second therapeutic agent.

Embodiment 327. A method of treating an MTAP-deficient and/or an MTA-accumulating disease in a subject in need thereof by administering to the subject an effective amount (e.g., a therapeutically effective amount) of a compound of any one of embodiments 1 to 324, or a pharmaceutically acceptable salt thereof, or a pharmaceutically acceptable composition of embodiment 325.

Embodiment 328. The method of embodiment 327 wherein the compound, or a pharmaceutically acceptable salt thereof, or composition is administered in combination with a second therapeutic agent.

Embodiment 329. A method of treating an MTAP-deficient and/or an MTA-accumulating disease in a subject in need thereof by administering to the subject an effective amount (e.g., a therapeutically effective amount) of a pharmaceutically acceptable composition of embodiment 326.

Embodiment 330. The method of any one of embodiments 327 to 329 wherein the disease is a proliferating disease.

Embodiment 331. The method of embodiment 330 wherein the disease is an MTAP-deficient and/or MTA-accumulating cancer.

Embodiment 332. The method of embodiment 331 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Embodiment 333.A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering a therapeutically effective amount of a compound of any one of embodiments 1 to 324, or a pharmaceutically acceptable salt thereof, or composition of embodiment 325 or 326 to the subject identified in step b).

Embodiment 334. Use of a compound of any one of embodiments 1 to 324, or a pharmaceutically acceptable salt thereof, or of a pharmaceutically acceptable composition of embodiment 325 for treating an MTAP-deficient and/or an MTA-accumulating disease in a subject in need thereof.

Embodiment 335. The use of embodiment 334 wherein the compound, or a pharmaceutically acceptable salt thereof, or composition is configured to be administered in combination with a second therapeutic agent.

Embodiment 336. Use of a pharmaceutically acceptable composition of embodiment 326 for treating an MTAP-deficient and/or an MTA-accumulating disease in a subject in need thereof.

Embodiment 337. The use of any one of embodiments 334 to 336 wherein the disease is a proliferating disease.

Embodiment 338. The use of embodiment 337 wherein the disease is an MTAP-deficient and/or MTA-accumulating cancer.

Embodiment 339. The use of embodiment 338 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Embodiment 340.A compound of any one of embodiments 1 to 324, or a pharmaceutically acceptable salt thereof, or a pharmaceutically acceptable composition of embodiment 325 for treating an MTAP-deficient and/or an MTA-accumulating disease in a subject in need thereof.

Embodiment 341. The compound or composition for use of embodiment 340 wherein the compound, or a pharmaceutically acceptable salt thereof, or composition is configured to be administered in combination with a second therapeutic agent.

Embodiment 342.A pharmaceutically acceptable composition of embodiment 326 for use in treating an MTAP-deficient and/or an MTA-accumulating disease in a subject in need thereof.

Embodiment 343. The compound or composition for use of any one of embodiments 340 to 342 wherein the disease is a proliferating disease.

Embodiment 344. The compound or composition for use of embodiment 343 wherein the disease is an MTAP-deficient and/or MTA-accumulating cancer.

Embodiment 345. The compound or composition for use of embodiment 344 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Embodiment 346. Use of a compound of any one of embodiments 1 to 324, or a pharmaceutically acceptable salt thereof, or of a pharmaceutically acceptable composition of embodiment 325 in the manufacturing of a medicament for treating an MTAP-deficient and/or an MTA-accumulating disease in a subject in need thereof.

Embodiment 347. The use of embodiment 346 wherein the medicament is configured to be administered in combination with a second therapeutic agent.

Embodiment 348. Use of a pharmaceutically acceptable composition of embodiment 326 in the manufacturing of a medicament for treating an MTAP-deficient and/or an MTA-accumulating disease in a subject in need thereof.

Embodiment 349. The use of any one of embodiments 346 to 348 wherein the disease is a proliferating disease.

Embodiment 350. The use of embodiment 349 wherein the disease is an MTAP-deficient and/or MTA-accumulating cancer.

Embodiment 351. The use of embodiment 350 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

### Examples

In order that the invention(s) described herein may be more fully understood, the following examples are set forth. The synthetic and biological examples described in this application are offered to illustrate the compounds, pharmaceutical compositions, and methods provided herein and are not to be construed in any way as limiting their scope. In the synthetic examples below, the descriptions of experimental procedures within a reaction sequence are listed in numerical order.

### Abbreviations

### General

ADDP 1,1′-(azodicarbonyl)dipiperidine
anhy. anhydrous
aq. aqueous
satd. saturated
min(s) minute(s)
hr(s) hour(s)
mL milliliter
mmolmillimole(s)
mol mole(s)
MS mass spectrometry
NMR nuclear magnetic resonance
TLC thin layer chromatography
HPLC high-performance liquid chromatography
Me methyl
i-Pr iso-propyl
t-Bu tert-butyl
^{t}BuXPhos 2-di-tert-butylphosphino-2’,4′,6′-triisopropylbiphenyl
Ph phenyl
Et ethyl
Bz benzoyl
RuPhos 2-dicyclohexylphosphino-2′,6′-diisopropoxy biphenyl
Spectrum
Hzhertz
δ chemical shift
J coupling constant
s singlet
d doublet
t triplet
q quartet
m multiplet
br broad
qd quartet of doublets
dquin doublet of quintets
dd doublet of doublets
dt doublet of triplets

### Solvents and Reagents

DAST Diethylaminosulfurtrifluoride
CHCl₃ chloroform
DCM dichloromethane
DMF dimethylformamide
Et₂O diethyl ether
EtOH ethyl alcohol
EtOAc ethyl acetate
MeOH methyl alcohol
MeCN acetonitrile
PE petroleum ether
THF tetrahydrofuran
DMSO dimethyl sulfoxide
t-BuOK potassium tert-butoxide
9-BBN 9-borabicyclo[3.3.1]nonane
AcOH acetic acid
HCl hydrochloric acid
H₂SO₄ sulfuric acid
NH₄Cl ammonium chloride
KOH potassium hydroxide
NaOH sodium hydroxide
K₂CO₃ potassium carbonate
Na₂CO₃ sodium carbonate
TFA trifluoroacetic acid
Na₂SO₄ sodium sulfate
NaBH₄ sodium borohydride
NaHCO₃sodium bicarbonate
LiHMDS lithium hexamethyldisilylamide
NaBH₄ sodium borohydride
Et₃N triethylamine
Py pyridine
PCC pyridinium chlorochromate
DMAP 4-(dimethylamino)pyridine
DIPEA N,N-diisopropylethylamine
BINAP 2,2′-bis(diphenylphosphanyl)-1,1′-binaphthyl
dppf 1,1′-bis(diphenylphosphino) ferrocene
PEP Phospho (enol) pyruvic acid
LDH Lactate Dehydrogenase
DTT DL-Dithiothreitol
BSA Bovine Serum Albumin
NADH β-Nicotinamide adenine dinucleotide, reduced
Pd(t-Bu₃P)₂ bis(tri-tert-butylphosphine) palladium (0)
AcCl acetyl chloride
i-PrMgCl Isopropylmagnesium chloride
TBSCI tert-Butyl(chloro) dimethylsilane
(i-PrO)₄Ti titanium tetraisopropoxide
BHT 2,6-di-t-butyl-4-methylphenoxide
BzCl benzoyl chloride
CsF cesium fluoride
DCC dicyclohexylcarbodiimide
DMP Dess-Martin periodinane
EtMgBr ethylmagnesium bromide
EtOAc ethyl acetate
TEA triethylamine
AlaOH alanine
TBAF tetra-n-butylammonium fluoride
TBS t-butyldimethylsilyl
TMS trimethylsilyl
TMSCF₃ (Trifluoromethyl)trimethylsilane
Ts p-toluenesulfonyl
Bu butyl
Ti(OⁱPr)₄ tetraisopropoxytitanium
LAH Lithium Aluminium Hydride
LDA lithium diisopropylamide
LiOH·H₂O lithium hydroxide hydrates
MAD methyl aluminum bis(2,6-di-t-butyl-4-methylphenoxide)
NBS N-bromosuccinimide
Na₂SO₄ sodium sulfate
Na₂S₂O₃ sodium thiosulfate
PE petroleum ether
MeCN acetonitrile
Boc t-butoxycarbonyl
MTBE methyl tert-butyl ether
DIAD diisopropyl azodicarboxylate

### General Experimental Notes:

In the following examples, the chemical reagents were purchased from commercial sources (such as Alfa, Acros, Sigma Aldrich, TCI and Shanghai Chemical Reagent Company), and used without further purification.

In some examples, purification of intermediates and final compounds was performed using HPLC (H₂O-MeOH; Agilent 1260 Infinity systems equipped with DAD and mass-detectors. Waters Sunfire C18 OBD Prep Column, 100 Å, 5 μm, 19 mm×100 mm with SunFire C18 Prep Guard Cartridge, 100 Å, 10 μm, 19 mm×10 mm) The material was dissolved in 0.7 mL DMSO. Flow: 30 mL/min. Purity of the obtained fractions was checked via the analytical LCMS. Spectra were recorded for each fraction as it was obtained straight after chromatography in the solution form. The solvent was evaporated under the N2 flow upon heating to 80° C. On the basis of post-chromatography LCMS analysis fractions were united. Solid fractions were dissolved in 0.5 mL MeOH and transferred into pre-weighted marked vials. Obtained solutions were again evaporated under the N₂ flow upon heating to 80° C. After drying, products were subjected to lyophilization using acetonitrile-water mixtures and finally characterized by LCMS and ¹H NMR.

Nuclear magnetic resonance (NMR) spectra were recorded using Brucker AVANCE DRX 500, Bruker 400 spectrometer or Varian UNITYplus 400. Chemical shifts for protons were reported as parts per million in δ scale using solvent residual peak (CHCl₃: 7.27 ppm) (methanol-d₄: 3.31 ppm)(DMSO-d₆: 2.50 ppm) or tetramethylsilane (0.00 ppm) as internal standards. Chemical shifts of ¹³C NMR spectra were reported in ppm from the central peak of CDCl₃ (77.00 ppm)(methanol-d₄: 49.15 ppm)(DMSO-d₆: 39.51 ppm) on the δ scale. Data are represented as follows: chemical shift, multiplicity (s=singlet, d=doublet, t=triplet, q=quartet, qn=quintuplet, sx=sextet, sp=septuplet, m=multiplet, br=broad), coupling constant (J, Hz) and integration.

In certain examples, mass spectra were recorded on an Agilent 1100 Series LC/MSD system with DAD\ELSD and Agilent LC\MSD VL (G1956A), SL (G1956B) mass-spectrometer or an Agilent 1200 Series LC/MSD system with DAD\ELSD and Agilent LC\MSD SL (G6130A), SL (G6140A) mass-spectrometer.

All the LC/MS data were obtained using positive/negative mode switching.

Column Zorbax SB-C18 1.8 μm 4.6×15 mm Rapid Resolution cartridge (PN 821975-932)
Mobile phase A—acetonitrile, 0.1% formic acid
   B—water (0.1% formic acid)
Flow rate 3 ml/min
Gradient 0 min—100% B
0.01 min—100% B
1.5 min—0% B
1.8 min—0% B
1.81 min—100% B
Injection volume 1 μl
Ionization mode atmospheric pressure chemical ionization (APCI)
Scan range m/z 80-1000.

### Other Exemplary Analytical LC/MS Instruments and Conditions are Described Below:

Instrument: Agilent LC1100-MS6100 series G1956B; Column: Xbridge Shield RP-18, 50*2.1 mm*5 μm; Mobile Phase A: H₂O with 0.05% NH₃—H₂O(v %); Mobile Phase B: MeCN; Flow rate: 1.0 mL/min; Wavelength: UV 220 nm, 254 nm; Column temperature: 30° C.; MS ionization: ESI.
0-30CD: Gradient: B from 0%~30% over 2 minutes and holding at 30% for 0.48 minutes;
0-60CD: Gradient: B from 0%~60% over 2 minutes and holding at 60% for 0.48 minutes;
10-80CD: Gradient: B from 10%~80% over 2 minutes and holding at 80% for 0.48 minutes;
30-90CD: Gradient: B from 30%~90% over 2 minutes and holding at 90% for 0.48 minutes;
50-100CD: Gradient: B from 50%~100% over 2 minutes and holding at 100% for 0.48 minutes.

Instrument: Agilent LC1100-MS6100 series G1956B; Column: Xtimate C18, 30*2.1 mm*3 μm; Mobile Phase A: H₂O with 0.0375% TFA (v %); Mobile Phase B: MeCN with 0.01875% TFA (v %): Flow rate: 0.8 mL/min; Wavelength: UV 220 nm, 254 nm; Column temperature: 50° C.; MS ionization: ESI.
0-30AB: Gradient: B from 0%~30% over 3 minutes and holding at 30% for 0.5 minutes;
0-60AB: Gradient: B from 0%~60% over 3 minutes and holding at 30% for 0.5 minutes;
10-80AB: Gradient: B from 10%~80% over 3 minutes and holding at 30% for 0.5 minutes;
30-90AB: Gradient: B from 0%~30% over 3 minutes and holding at 30% for 0.5 minutes;
50-100AB: Gradient: B from 50%~100% over 3 minutes and holding at 100% for 0.5 minutes.

Instrument: Shimadzu LC20-MS2010; Column: Agilent Pursit 5 C18 20*2.0 mm; Mobile Phase A: H₂O with 0.0375% of TFA (v %); Mobile Phase B: MeCN with 0.01875% of TFA (v %); Gradient: B from 5~95% over 0.7 minutes and holding at 95% for 0.4 minutes; Flow Rate: 1.5 mL/min; Wavelength: UV 220 nm, 254 nm, 215 nm; Column temperature: 50° C.; MS ionization: ESI.

Instrument: Shimadzu LC20-MS2020; Column: Agilent Pursit 5 C18 20*2.0 mm; Mobile Phase A: H₂O with 0.0375% of TFA (v %); Mobile Phase B: MeCN with 0.01875% of TFA (v %); Gradient: B from 5~95% over 0.7 minutes and holding at 95% for 0.4 minutes; Flow Rate: 1.5 mL/min; Wavelength: UV 220 nm, 254 nm; Column temperature: 50° C.; MS ionization: ESI.

### Exemplary HPLC Instruments and Conditions

Instrument: Shimadzu LC20; Column: YMC-Pack ODS-A 150*4.6 mm; Mobile Phase A: H₂O with 0.06875% TFA (v %); Mobile Phase B: MeCN with 0.0625% TFA (v %); Flow rate: 1.5 mL/min; Wavelength: UV 220 nm, 215 nm, 254 nm; Column temperature: 40° C.
0-30: Gradient: B from 0~30% over 10 minutes and holding at 30% for 5 minutes;
0~60: Gradient: B from 0~60% over 10 minutes and holding at 60% for 5 minutes;
0-95: Gradient: B from 0~95% over 10 minutes and holding at 95% for 5 minutes;
10-80: Gradient: B from 10~80% over 10 minutes and holding at 80% for 5 minutes;
30-90: Gradient: B from 30~90% over 10 minutes and holding at 90% for 5 minutes;
50-100: Gradient: B from 50~100% over 10 minutes and holding at 100% for 5 minutes.

Instrument: Shimadzu LC20; Column: Xbridge Shield RP-18 50*2.1 mm, 5 μm; Mobile Phase A: H₂O with 0.01% NH₃—H₂O; Mobile Phase B: MeCN; Flow Rate: 1.2 mL/min; Wavelength: UV 220 nm, 215 nm, 254 nm; Column temperature: 40° C.
0-30CD: Gradient: B from 0~30% over 6 minutes and holding at 30% for 2 minutes;
0-60CD: Gradient: B from 0~60% over 6 minutes and holding at 60% for 2 minutes;
10-80CD: Gradient: B from 10~80% over 6 minutes and holding at 80% for 2 minutes;
30-90CD: Gradient: B from 30~90% over 6 minutes and holding at 90% for 2 minutes;
50-100CD: Gradient: B from 10~80% over 6 minutes and holding at 100% for 2 minutes.

Instrument: Shimadzu LC20; Column: Ultimate C18 50*3 mm, 3 μm; Mobile Phase A: H₂O with 0.06875% TFA (v %); Mobile Phase B: MeCN with 0.0625% TFA (v %); Flow Rate: 1.2 mL/min; Wavelength: UV 220 nm, 215 nm, 254 nm; Column temperature: 40° C.
0-30AB: Gradient: B from 0~30% over 2.5 minutes and holding at 30% for 0.75 minutes;
0-60AB: Gradient: B from 0~60% over 2.5 minutes and holding at 60% for 0.75 minutes;
5-95AB: Gradient: B from 5~95% over 2.5 minutes and holding at 95% for 0.75 minutes.

Instrument: Shimadzu LC20; Column: Ultimate C18 50*3 mm, 3 μm; Mobile Phase A: H₂O with 0.06875% TFA (v %); Mobile Phase B: MeCN with 0.0625% TFA (v %); Flow Rate: 1.2 mL/min; Wavelength: UV 220 nm, 215 nm, 254 nm; Column temperature: 40° C.
10-80AB: Gradient: B from 10~80% over 4 minutes and holding at 80% for 2 minutes.

### Exemplary TLC, Concentration and Normal Phase Chromatography.

Analytical thin layer chromatography (TLC) was performed with silica gel 60 F254 aluminum plates. Visualization was done under a UV lamp (254 nm) and by iodine or immersion in ethanolic phosphomolybdic acid (PMA) or potassium permanganate (KMnO₄), followed by heating using a heat gun. Organic solutions were concentrated by rotary evaporation at 20~40° C. Purification of reaction products were generally done by flash column chromatography with 230-400 mesh silica gel or Agela flash silica column.

### Exemplary Chiral SFC Analytical Methods

Column: Chiralpak AD-3 150×4.6 mm I.D., 3 μm; Mobile phase: A: supercritical CO₂; Mobile phase B: EtOH (0.05% DEA); Gradient: from 5% to 40% of B in 5 min and hold 40% for 2.5 min, then 5% of B for 2.5 min; Flow rate: 2.5 mL/min; Column temperature: 35° C.; ABPR: 1500 psi.

Column: Chiralpak AD-3 100×4.6 mm I.D., 3 μm; Mobile phase: A: supercritical CO₂ Mobile phase B: EtOH (0.1% ethanolamine); Gradient: from 5% to 40% of B in 4.5 min and hold 40% for 2.5 min, then 5% of B for 1 min; Flow rate: 2.8 mL/min; Column temperature: 40° C.

### Exemplary Preparative HPLC Separation Methods

Basic condition (NH₃—H₂O): Instrument: Gilson GX-281 Liquid Handler, Gilson 322 Pump, Gilson 156 UV Detector; Column: Waters Xbridge 150×25 mm×5 μm; Mobile phase A: H₂O with 0.05% NH₃—H₂O(v %); Mobile phase B: MeCN; Gradient: B from 22% to 52% in 9.5 min, hold 100% B for 1 min; Flow Rate: 25 mL/min; Column Temperature: 30° C.; Wavelength: 220 nm, 254 nm.

Acid condition (HCOOH): Instrument: Gilson GX-281 Liquid Handler, Gilson 322 Pump, Gilson 156 UV Detector; Column: Agela Durashell C18 150*25 mm 5 μm; Mobile phase A: H₂O(0.0225% HCOOH); Mobile phase B: MeCN; Gradient: B from 7% to 37% in 9 min, hold 100% B for 0 min; Flow Rate: 25 mL/min; Column Temperature: 30° C.; Wavelength: 220 nm, 254 nm.

Acid condition (HCl): Gilson GX-281 Liquid Handler, Gilson 322 Pump, Gilson 156 UV Detector; Column: Xtimate C18 150*25 mm*5 μm; Mobile phase A: H₂O with 0.05% HCl (v %); Mobile phase B: MeCN; Gradient: B from 0% to 30% in 6.5 min, hold 100% B for 2.5 min; Flow Rate: 25 mL/min; Column Temperature: 30° C.; Wavelength: 220 nm, 254 nm).

Neutral condition (NH₄HCO₃):(Instrument: Gilson GX-281 Liquid Handler, Gilson 322 Pump, Gilson 156 UV Detector; Column: Waters Xbridge 150×25 mm×5 μm; Mobile phase A: H₂O with 10 mmol NH₄HCO₃; Mobile phase B: MeCN; Gradient: B from 39% to 69% in 10 min, hold 100% B for 2.5 min; Flow Rate: 25 mL/min; Column Temperature: 30° C.; Wavelength: 220 nm, 254 nm).

### Exemplary Large-Scale Separation

Basic condition: Instrument: Shimadzu LC-8A Pumps, Shimadzu SCL-10A VP System Controller, Shimadzu SPD-20AV UV/VIS Detector; Column: Phenomenex Gemini C18 250*50 mm*10 μm; Mobile phase A: water (0.04% NH₃-H₂O+10 mM NH₄HCO₃); Mobile phase B: MeCN; Gradient: B from 65% to 95% in 26 min, hold 100% B for 3 min; Flow Rate: 110 mL/min; Column Temperature: 30° C.; Wavelength: 220 nm, 254 nm.

Acid condition (TFA): Instrument: Shimadzu LC-20AP Pumps, Shimadzu CBM-20A System Controller Shimadzu SPD-20AV UV/VIS Detector; Column: Phenomenex luna C18 250×50 mm×10 μm; Mobile phase A: H₂O with 0.1% TFA (v %); Mobile phase B: MeCN; Gradient: B from 0% to 25% in 15 min, hold 100% B for 4 min; Flow Rate: 120 mL/min; Column Temperature: 30° C.; Wavelength: 220 nm, 254 nm.

### Exemplary Preparative Chiral SFC Method:

Exemplary chiral columns available for use in the separation/purification of the enantiomers/diastereomers provided herein include, but are not limited to, CHIRALPAK® AD-10, CHIRALCEL® OB, CHIRALCEL® OB—H, CHIRALCEL® OD, CHIRALCEL® OD-H, CHIRALCEL® OF, CHIRALCEL® OG, CHIRALCEL® OJ and CHIRALCEL® OK.

In certain examples, the chiral separation was performed under the following conditions: Instrument: Thar 80; Column: Daicel Chiralpak AD. 250×30 mm I.D. 10 μm; Mobile phase: supercritical CO₂/MeOH (0.1% NH₃—H₂O, v %)=60/40; Flow Rate: 70 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative pyrazoles that have been listed herein. The compounds provided herein may be prepared from known or commercially available starting materials and reagents by one skilled in the art of organic synthesis.

Exemplary general method for preparative HPLC: Column: Waters RBridge prep 10 μm C18, 19*250 mm. Mobile phase: acetonitrile, water (NH₄HCO₃)(30 L water, 24 g NH₄HCO₃, 30 mL NH₃·H₂O). Flow rate: 25 mL/min.

Exemplary general method for analytical HPLC: Mobile phase: A: water (10 mM NH₄HCO₃), B: acetonitrile Gradient: 5%- 95% B in 1.6 or 2 min Flow rate: 1.8 or 2 mL/min; Column: XBridge C18, 4.6*50 mm, 3.5 μm at 45° C.

### Intermediates

### Intermediate 1. (R)-1-cyclopropyl-2-methoxy-N-((5-(trifluoromethyl)pyridin-2-yl)methyl)ethanamine

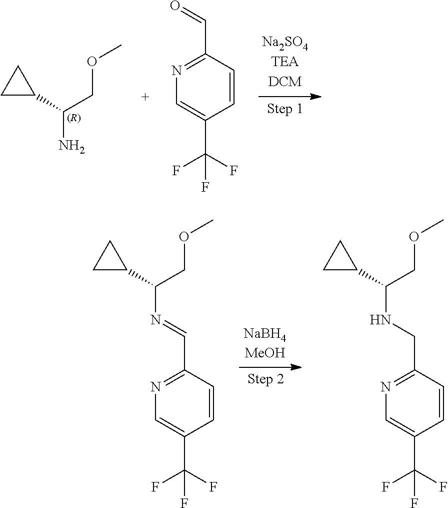

### Step 1:1-cyclopropyl-2-methoxy-N-((5-(trifluoromethyl)pyridin-2-yl)methylene)ethanamine

To a stirred solution of (1R)-1-cyclopropyl-2-methoxy-ethanamine (0.8 g, 5.28 mmol, HCl) and 5-(trifluoromethyl)pyridine-2-carbaldehyde (987.41 mg, 5.64 mmol) in CH₂Cl₂ (10 mL) were added Triethylamine (726.00 mg, 7.17 mmol, 1 mL) and Sodium sulfate anhydrous (3.80 g, 26.74 mmol, 1.42 mL). The resulting reaction mixture was stirred at 25° C. for 16 h. Upon completion, the reaction mixture was evaporated, quenched with water (20 mL). The aqueous phase was extracted with CHCl₃ (2×20 mL). The combined organic phase was dried over Na₂SO₄ and concentrated under reduced pressure. The desired product (E)-1-cyclopropyl-2-methoxy-N-((5-(trifluoromethyl)pyridin-2-yl)methylene)ethanamine (1.4 g, 5.14 mmol, 97.46% yield) was isolated.

### Step 2:1-cyclopropyl-2-methoxy-N-((5-(trifluoromethyl)pyridin-2-yl)methyl)ethanamine

To a stirred solution of (E)-N-[(1R)-1-cyclopropyl-2-methoxy-ethyl]-1-[5-(trifluoromethyl)-2-pyridyl]methanimine (1.4 g, 5.14 mmol) in MeOH (30 mL) was added Sodium Borohydride (0.6 g, 15.86 mmol, 558.66 μL). The resulting reaction mixture was stirred at 25° C. for 16 hr. Upon completion, the reaction mixture was evaporated, quenched with water (20 mL). The aqueous phase was extracted with CHCl₃ (2×20 mL). The combined organic phase was dried over Na₂SO₄ and concentrated under reduced pressure. The desired product 1-cyclopropyl-2-methoxy-N-((5-(trifluoromethyl)pyridin-2-yl)methyl)ethanamine (1.4 g, 5.10 mmol, 99.27% yield) was isolated.

### Intermediate 2. Synthesis of (1R)—N-[[5-(trifluoromethyl)-2-pyridyl]methyl]indan-1-amine

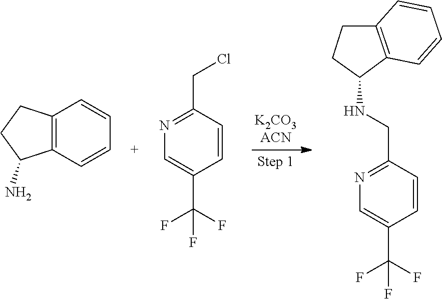 [SMILES: N[C@@H]1CCc2ccccc21 ; FC(F)(F)c1ccc(CCl)nc1 ; FC(F)(F)c1ccc(CN[C@@H]2CCc3ccccc32)nc1]

To a solution of (1R)-indan-1-amine (2.75 g, 16.21 mmol, HCl) and 2-(chloromethyl)-5-(trifluoromethyl)pyridine (3.17 g, 16.21 mmol) in ACN(50 mL) was added potassium carbonate (9.33 g, 40.52 mmol, 4.08 mL, 60% purity). The resulting mixture was stirred at 60° C. for 12 hr and evaporated in vacuo. The residue was diluted with water (100 mL) and extracted with dichloromethane (2*100 mL). The combined organic extracts were dried over sodium sulphate and evaporated in vacuo. The residue was purified by silica gel flash chromatography eluting with a 0 to 100 percent DCM-methanol gradient to afford N-[5-(trifluoromethyl)-2-pyridyl]methyl]indan-1-amine (3.2 g, 10.95 mmol, 67.54% yield).

LCMS (ESI): [M+H]⁺m/z: calcd 293.1; found 293.0; Rt=0.698 min.

### Intermediate 3. (1R,2R)-2-(difluoromethoxy)-N-((5-(trifluoromethyl)pyridin-2-yl)methyl)cyclopentanamine

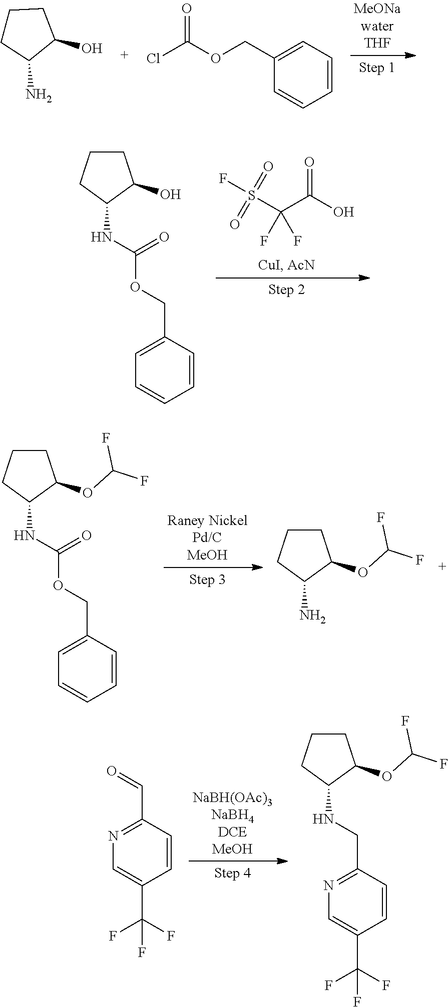

### Step 1: benzyl((1R,2R)-2-hydroxycyclopentyl)carbamate

(1R,2R)-2-aminocyclopentanol (35.30 g, 256.53 mmol, HCl) was dissolved in Water (300 mL) and Sodium hydrogen carbonate, 99% (53.87 g, 641.32 mmol, 24.95 mL) was added portionwise. THF (250 mL) was added to the previous mixture and the resulting mixture was cooled to 0° C. in an ice/methanol bath. A solution of benzyl carbonochloridate (45.95 g, 269.35 mmol, 38.45 mL) was added dropwise at 0° C. and the resulting mixture was allowed to warm to room temperature and stirred overnight. An organic layer was separated and the aqueous layer was extracted with EtOAc (2*200 ml). Combined organic layers were concentrated in vacuo and the residue was re-dissolved in MTBE (350 ml). The resulting solution was washed with water (2×200 ml), brine (150 ml), dried over Na₂SO₄, filtered and concentrated in vacuo to obtain benzyl((1R,2R)-2-hydroxycyclopentyl)carbamate (50.93 g, 216.47 mmol, 84.38% yield) which was used in the next step without purification.

LCMS (ESI): [M+H]⁺m/z: calcd 236.1; found 236.0; Rt=1.084 min.

### Step 2: benzyl((1R,2R)-2-(difluoromethoxy)cyclopentyl)carbamate

Benzyl N-[(1R,2R)-2-hydroxycyclopentyl]carbamate (50.93 g, 216.47 mmol) was dissolved in MeCN(500 mL) and Copper (I) iodide (8.25 g, 43.29 mmol, 1.47 mL) was added thereto. The resulting mixture was heated to 50° C. and a solution of 2,2-difluoro-2-fluorosulfonyl-acetic acid (57.82 g, 324.70 mmol, 33.55 mL) in MeCN(50 mL) was added dropwise at 50° C. After addition completed, the reaction mixture was heated at 50° C. for 30 min. The reaction mixture was concentrated in vacuo and the residue was dissolved in a mixture of hexane and EtOAc (1:1, 300 ml). The resulting mixture was filtered through a silica gel pad and the SiO₂ pad was rinsed with a mixture of hexane and EtOAc (1:1, 1000 ml). The filtrate was concentrated in vacuo and the residue was purified by column chromatography (hexane-EtOAc 5:1) to obtain benzyl((1R,2R)-2-(difluoromethoxy)cyclopentyl)carbamate (33.9 g, 118.83 mmol, 54.89% yield).

### Step 3: (1R,2R)-2-(difluoromethoxy)cyclopentanamine

benzyl N-[(1R,2R)-2-(difluoromethoxy)cyclopentyl]carbamate (33.90 g, 118.83 mmol) was dissolved in MeOH (350 mL) and Raney Nickel (20.92 g, 356.48 mmol)(fresh prepared, slurry in MeOH) was added thereto. The resulting suspension was stirred overnight. The catalyst was filtered and the filtrate was concentrated in vacuo. The residue was dissolved in MeOH (350 mL) and Palladium, 10% on carbon, Type 487, dry (6.32 g, 5.94 mmol, 10% purity) was added thereto. The resulting suspension was evacuated and backfilled three times with hydrogen and the resulting mixture was hydrogenated at 1 atm (balloon) over the weekend. The catalyst was filtered off and the filtrate was concentrated in vacuo. The residue was dissolved in MTBE (150 ml) and the resulting solution was washed with 15% NaOH solution (2×150 ml). The organic layer was dried over Na₂SO₄, filtered and concentrated in vacuo to obtain (1R,2R)-2-(difluoromethoxy)cyclopentanamine (12 g, 79.39 mmol, 66.81% yield).

### Step 4: (1R,2R)-2-(difluoromethoxy)-N-((5-(trifluoromethyl)pyridin-2-yl)methyl)cyclopentanamine

(1R,2R)-2-(difluoromethoxy)cyclopentanamine (500.00 mg, 3.31 mmol) was dissolved in DCE (10 mL) and 5-(trifluoromethyl)pyridine-2-carbaldehyde (579.24 mg, 3.31 mmol) was added thereto. The resulting mixture was stirred for 30 min and Sodium triacetoxyborohydride, 95% (1.40 g, 6.62 mmol) was added portionwise to the previous solution, the resulting mixture was stirred overnight. Aq. NaHCO₃ solution (20 mL) was added to the reaction mixture and an organic layer was separated. The aqueous layer was extracted with DCM (25 mL) and combined organic layers were washed with aq. NaHCO₃ solution (25 mL), dried over Na₂SO₄, filtered and concentrated in vacuo. The reaction mixture was dissolved in MeOH (20 mL) and Sodium Borohydride (375.44 mg, 9.92 mmol, 349.57 μL) was added thereto. The resulting mixture was stirred for 1 hr. The reaction mixture was concentrated in vacuo and water (30 mL) was added thereto. The resulting mixture was extracted with DCM (2×25 mL) and combined organic layers were dried over Na₂SO₄, filtered and concentrated in vacuo to obtain (1R,2R)-2-(difluoromethoxy)-N-((5-(trifluoromethyl)pyridin-2-yl)methyl)cyclopentanamine (1.06 g, crude) which was used further without purification.

LCMS (ESI): [M+H]⁺m/z: calcd 311.1; found 311.0; Rt=0.829 min.

### Intermediate 4. (rac)-4-methyl-N-[[5-(trifluoromethyl)-2-pyridyl]methyl]tetralin-1-amine

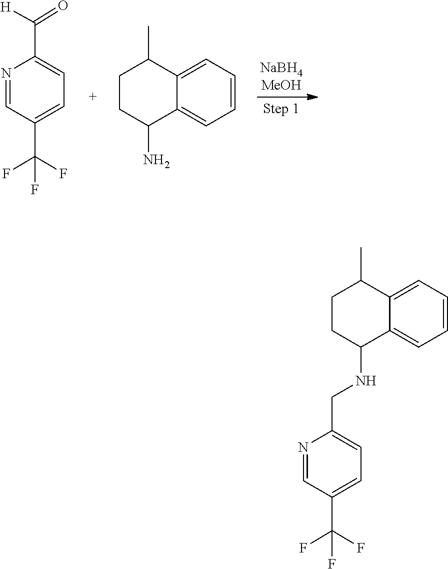 [SMILES: O=Cc1ccc(C(F)(F)F)cn1 ; CC1CCC(N)c2ccccc21 ; CC1CCC(NCc2ccc(C(F)(F)F)cn2)c2ccccc21]

5-(trifluoromethyl)pyridine-2-carbaldehyde (2.1 g, 11.99 mmol) 5-(trifluoromethyl)pyridine-2-carbaldehyde (2.1 g, 11.99 mmol) was added to a stirred solution of 4-methyltetralin-1-amine (1.93 g, 11.99 mmol) in Methanol (50 mL) stirred at 20° C. for 2 hr and Sodium Borohydride (226.86 mg, 6.00 mmol, 211.23 μL) added, reaction mixture stirred for 2 hr additional. The reaction mixture was evaporated in vacuo. The residue was dissolved in DCM (50 ml) and washed with water (2*15 ml). Organic layer was dried over Na₂SO₄ and filtered. DCM was evaporated under reduce pressure to give (rac)-4-methyl-N-[[5-(trifluoromethyl)-2-pyridyl]methyl]tetralin-1-amine (2.9 g, 9.05 mmol, 75.48% yield).

LCMS (ESI): [M+H]⁺m/z: calcd 321.2; found 321.2; Rt=0.981 min.

### Intermediate 5. 3-methyl-N-[[5-(trifluoromethyl)-2-pyridyl]methyl]butan-2-amine)

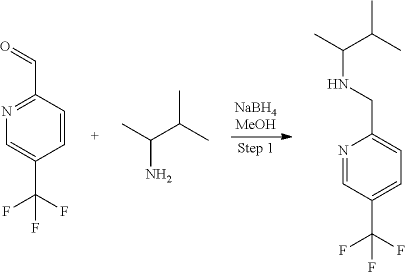

A solution of 5-(trifluoromethyl)pyridine-2-carbaldehyde (1 g, 5.71 mmol) and 3-methylbutan-2-amine (796.43 mg, 9.14 mmol) in Methanol (25 mL) was stirred at 25° C. for 12 h. To this solution, Sodium Borohydride (86.42 mg, 2.28 mmol, 80.47 μL) was added and the resulting mixture was stirred for 2 hr. The solvent was removed in vacuo, the residue was taken up with water (10 ml) and extracted with DCM (3*20 ml). The combined organic layer was washed with brine (10 ml), dried over Na₂SO₄ and evaporated to obtain 3-methyl-N-[5-(trifluoromethyl)-2-pyridyl]methyl]butan-2-amine (1.35 g, 5.48 mmol, 95.99% yield).

### Example 1. Synthesis of (R)-N1-(4-amino-1H-pyrazolo[4,3-c]pyridin-7-yl)-N2-(2,3-dihydro-1H-inden-1-yl)-N2-((5-(trifluoromethyl)pyridin-2-yl)methyl)oxalamide (Compound 8)

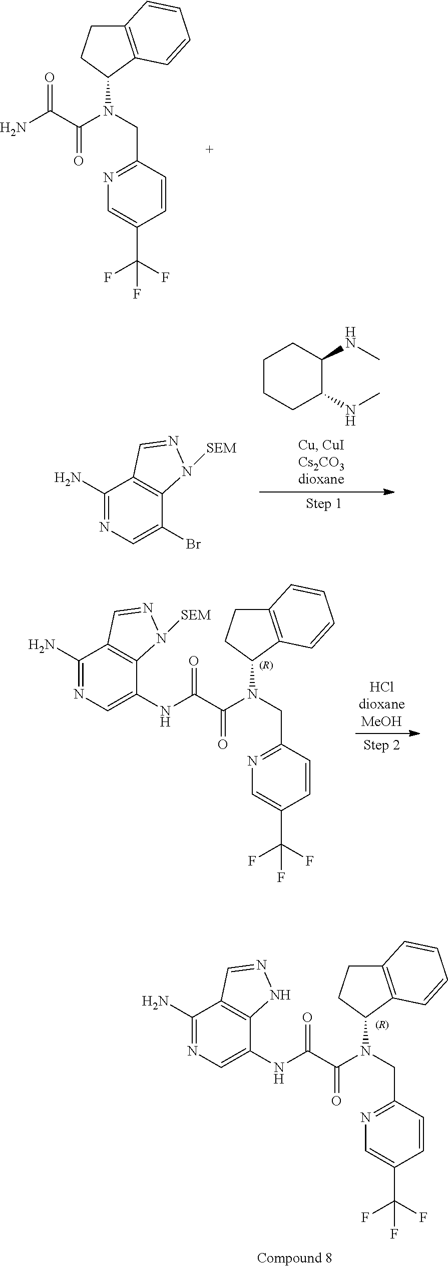

(R)—N¹-(2,3-dihydro-1H-inden-1-yl)-N1-((5-(trifluoromethyl)pyridin-2-yl)methyl)oxalamide is synthesized by the following procedure. To a solution of (IR)—N-[5-(trifluoromethyl)-2-pyridyl]methyl]indan-1-amine (2.3 g, 7.87 mmol) and TEA (1.59 g, 15.74 mmol, 2.19 mL) in THF (18.40 mL) was added 2,2,2-trifluoroethyl 2-chloro-2-oxo-acetate (2.25 g, 11.80 mmol) dropwise at 0° C. under argon. The reaction mixture was then stirred for 12 hr at r.t. Then ammonia (155.05 mg, 9.10 mmol) was bubbled through for 10 min at 0° C. The reaction mixture was then stirred for 12 hr at r.t. The reaction mixture was filtered off and the filtrate was evaporated in vacuo to give N′—[(IR)-indan-1-yl]-N′-[5-(trifluoromethyl)-2-pyridyl]methyl]oxamide (2 g, 5.50 mmol, 69.96% yield).

LCMS (ESI): [M+H]⁺m/z: calcd 364.1; found 364.0; Rt=1.286 min.

### Step 1: (R)—N¹-(4-amino-1-((2-(trimethylsilyl) ethoxy)methyl)-1H-pyrazolo[4,3-c]pyridin-7-yl)-N2-(2,3-dihydro-1H-inden-1-yl)-N2-((5-(trifluoromethyl)pyridin-2-yl)methyl)oxalamide

A mixture of N′—[(1R)-indan-1-yl]-N′-[5-(trifluoromethyl)-2-pyridyl]methyl]oxamide (380 mg, 836.70 μmol), 7-bromo-1-(2-trimethylsilylethoxymethyl)pyrazolo[4,3-c]pyridin-4-amine (300.00 mg, 873.89 μmol), copper (5 mg, 78.68 μmol), Copper (I) iodide (100 mg, 525.07 μmol, 17.79 μL), cesium carbonate (599.75 mg, 1.84 mmol) and rac-(1R,2R)—N1,N2-dimethylcyclohexane-1,2-diamine (100 mg, 703.04 μmol) in 1,4-dioxane (6.00 mL) was stirred in a sealed vial under argon at 105° C. for 48 hr. The resulting mixture was cooled down and submitted to reverse phase HPLC (column: Chromatorex 18 SMB100-5T 100×19 mm 5 μm; mobile phase: 40-40-90% 0-1-6 min H₂O/Acetonitrile, flow rate: 30 ml/min (loading pump 4 ml/min acetonitrile) to afford (R)—N¹-(4-amino-1-((2-(trimethylsilyl) ethoxy)methyl)-1H-pyrazolo[4,3-c]pyridin-7-yl)-N2-(2,3-dihydro-1H-inden-1-yl)-N2-((5-(trifluoromethyl)pyridin-2-yl)methyl)oxalamide (125 mg, 199.77 μmol, 23.88% yield) as a light-brown gum.

LCMS (ESI): [M+H]⁺m/z: calcd 626.3; found 626.2; Rt=1.286 min.

### Step 2: The synthesis of (R)—N¹-(4-amino-1H-pyrazolo[4,3-c]pyridin-7-yl)-N2-(2,3-dihydro-1H-inden-1-yl)-N2-((5-(trifluoromethyl)pyridin-2-yl)methyl)oxalamide

Hydrogen chloride solution 4.0M in dioxane (2.96 g, 11.28 mmol, 3.70 mL, 13.9% purity) was added to a stirred solution of N-[4-amino-1-(2-trimethylsilylethoxymethyl)pyrazolo[4,3-c]pyridin-7-yl]-N′—[(1R)-indan-1-yl]-N′-[5-(trifluoromethyl)-2-pyridyl]methyl]oxamide (125 mg, 199.77 μmol) in methanol (3.25 mL) at 25° C. The resulting mixture was stirred in a sealed vial at 25° C. for 15 hr, and then concentrated to dryness in vacuo. The residue was submitted to reverse phase HPLC (Chromatorex 18 SMB100-5T 100×19 mm 5 μm; mobile phase: 20-20-45% 0-1-5 min H₂O/ACN/0.1% FA; flow rate: 30 ml/min (loading pump 4 ml/min acetonitrile) to afford (R)—N¹-(4-amino-1H-pyrazolo[4,3-c]pyridin-7-yl)-N2-(2,3-dihydro-1H-inden-1-yl)-N2-((5-(trifluoromethyl)pyridin-2-yl)methyl)oxalamide (72 mg, 132.97 μmol, 66.56% yield, HCOOH) as a light-yellow solid.

LCMS (ESI): [M+H]⁺m/z: calcd 496.2; found 496.2; Rt=2.960 min.

### Example 2. Synthesis of N-(4-amino-1H-pyrazolo[4,3-c]pyridin-7-yl)-N′-(1,2-dimethylnonyl)-N′-115-(trifluoromethyl)-2-pyridyl]methyl]oxamide (Compound 18)

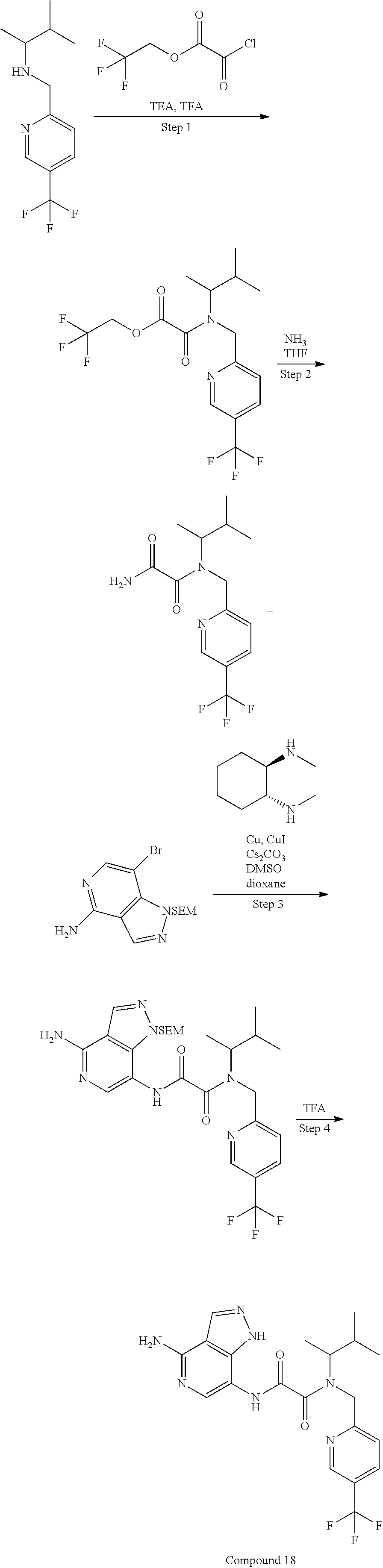

The synthesis of 3-methyl-N-((5-(trifluoromethyl)pyridin-2-yl)methyl) butan-2-amine is described in Intermediate 5.

### Step 1:2,2,2-trifluoroethyl 2-[1,2-dimethylpropyl-[[5-(trifluoromethyl)-2-pyridyl]methyl]amino]-2-oxo-acetate

To a solution of 3-methyl-N-[5-(trifluoromethyl)-2-pyridyl]methyl]butan-2-amine (0.6 g, 2.44 mmol) and Triethylamine (2.47 g, 24.36 mmol, 3.40 mL) in THF (28.30 mL), 2,2,2-trifluoroethyl 2-chloro-2-oxo-acetate (1.86 g, 9.75 mmol) was added portionwise at 0° C. The resulting mixture was stirred for 2 hr. LCMS showed full conversion of SM. The reaction mixture was directly used for the next step.

LCMS (ESI): [M+H]⁺m/z: calcd 401.1; found 401.0; Rt=1.543 min.

### Step 2: N′-(1,2-dimethylpropyl)-N′-[[5-(trifluoromethyl)-2-pyridyl]methyl]oxamide

Through a solution of 2,2,2-trifluoroethyl 2-[1,2-dimethylpropyl-[5-(trifluoromethyl)-2-pyridyl]methyl]amino]-2-oxo-acetate (0.97 g, 2.42 mmol) in THF (40 mL), ammonia was bubbled during 10 min at 0° C. The formed precipitate was filtered off, washed with THF (30 ml) and the solvent was evaporated in vacuo to give N′-(1,2-dimethylpropyl)-N′-[[5-(trifluoromethyl)-2-pyridyl]methyl]oxamide (0.7 g, 2.21 mmol, 91.04% yield).

LCMS (ESI): [M+H]⁺m/z: calcd 318.2; found 318.0; Rt=1.050 min.

### Step 3: N-[4-amino-1-(2-trimethylsilylethoxymethyl)pyrazolo[4,3-c]pyridin-7-yl]-N′-(1,2-dimethylpropyl)-N′-[[5-(trifluoromethyl)-2-pyridyl]methyl]oxamide

To a mixture of N′-(1,2-dimethylpropyl)-N′-[5-(trifluoromethyl)-2-pyridyl]methyl]oxamide (0.35 g, 1.10 mmol), 7-bromo-1-(2-trimethylsilylethoxymethyl)pyrazolo[4,3-c]pyridin-4-amine (454.40 mg, 1.32 mmol), Copper (14.02 mg, 220.61 μmol), Copper (I) iodide (210.07 mg, 1.10 mmol, 37.38 μL), Cesium carbonate (718.78 mg, 2.21 mmol) and rac-(1R,2R)—N1,N2-dimethylcyclohexane-1,2-diamine (235.34 mg, 1.65 mmol), Dioxane (6 mL) was added. The resulting mixture was evacuated, refiled with Argon three time, heated at 100° C. for 36 hr and cooled. The inorganic precipitate was filtered and solution was subjected to HPLC (Device (Mobile Phase, Column): SYSTEM 45-70% 0-5 min H₂O/ACN/0.1% NH₄OH, flow: 30 ml/min (loading pump 4 ml/min ACN) target mass 579.70 column: XBridge C18 100×19 mm, 5 μm) to give N-[4-amino-1-(2-trimethylsilylethoxymethyl)pyrazolo[4,3-c]pyridin-7-yl]-N′-(1,2-dimethylpropyl)-N′-[[5-(trifluoromethyl)-2-pyridyl]methyl]oxamide (140 mg, 241.51 μmol, 21.89% yield).

LCMS (ESI): [M+H]⁺m/z: calcd 580.3; found 580.4; Rt=1.397 min.

### Step 4: The synthesis of N-(4-amino-1H-pyrazolo[4,3-c]pyridin-7-yl)-N′-(1,2-dimethylpropyl)-N′-[[5-(trifluoromethyl)-2-pyridyl]methyl]oxamide

A solution of N-[4-amino-1-(2-trimethylsilylethoxymethyl)pyrazolo[4,3-c]pyridin-7-yl]-N′-(1,2-dimethylpropyl)-N′-[[5-(trifluoromethyl)-2-pyridyl]methyl]oxamide (140 mg, 241.51 μmol) in Trifluoroacetic acid (3 g, 26.31 mmol, 2.03 mL) was stirred at 25° C. for 12 hr. The solvent was evaporated in vacuo and the residue was purified by HPLC (Device (Mobile Phase, Column): SYSTEM 30-80% 0-5 min H₂O/MeOH/0.1% NH₄OH, flow: 30 ml/min (loading pump 4 ml/min ACN) target mass 449.44 column: XBridge C18 100×19 mm, 5 μm) to give N-(4-amino-1H-pyrazolo[4,3-c]pyridin-7-yl)-N′-(1,2-dimethylpropyl)-N′-[5-(trifluoromethyl)-2-pyridyl]methyl]oxamide (22 mg, 48.95 μmol, 20.27% yield) as a light-yellow solid.

¹H NMR (DMSO-d6, 600 MHz): δ (ppm) 0.74-1.21 (m, 9H), 1.85-1.96 (m, 1H), 3.92-4.12 (m, 1H), 4.50-4.64 (m, 1H), 4.79-4.84 (m, 1H), 6.59-6.67 (m, 2H), 7.51-7.76 (m, 2H), 8.11-8.20 (m, 2H), 8.72-8.90 (m, 1H), 10.25-10.46 (m, 1H), 12.64-12.75 (m, 1H).

LCMS (ESI): [M+H]⁺m/z: calcd 450.2; found 450.2; Rt=2.611 min.

### Example 3. Synthesis of N-(4-amino-1H-pyrazolo[4,3-c]pyridin-7-yl)-N′-benzyl-N′-(pyrimidin-2-ylmethyl)oxamide (Compound 27)

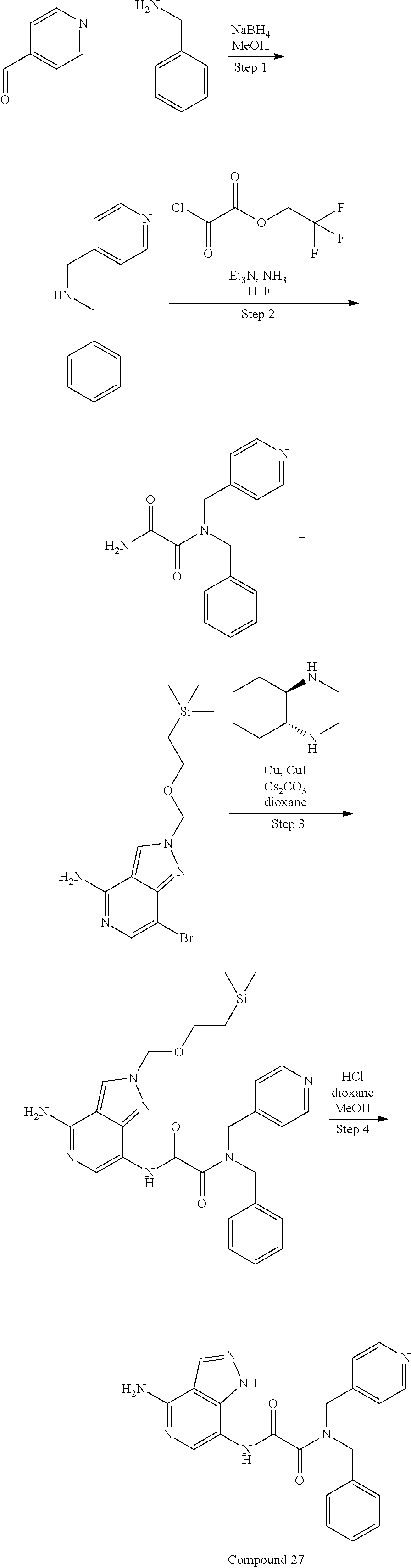

### Step 1:1-phenyl-N-(4-pyridylmethyl)methanamine

pyridine-4-carbaldehyde (1 g, 9.34 mmol, 883.39 μL) was added to a solution of phenylmethanamine (1.00 g, 9.34 mmol) in methanol (25 mL) at 25° C. The resulting mixture was stirred at 25° C. for 15 hr, then cooled to 0° C. and Sodium Borohydride (353.21 mg, 9.34 mmol, 328.88 μL) was added in one portion. The reaction mixture was allowed to warm to 25° C. and stirred for 1 hr, then concentrated in vacuo. The residue was diluted with water (25 ml) and extracted with dichloromethane (40 ml). The organic layer was separated, dried over sodium sulfate and concentrated in vacuo to afford crude 1-phenyl-N-(4-pyridylmethyl)methanamine (1.6 g, 8.07 mmol, 86.44% yield) as yellow oil, which was used directly in the next step.

LCMS (ESI): [M+H]⁺m/z: calcd 199.2; found 199.2; Rt=0.195 min.

### Step 2: N′-benzyl-N′-(4-pyridylmethyl)oxamide

2,2,2-trifluoroethyl 2-chloro-2-oxo-acetate (1.2 g, 6.30 mmol) was added slowly to a cooled to -10° C. mixture of 1-phenyl-N-(4-pyridylmethyl)methanamine (800 mg, 4.04 mmol) and triethyl amine (2 g, 19.76 mmol, 2.75 mL) in THF (50 mL). The resulting mixture was allowed to warm to 25° C. and stirred for 2 hr. Then gaseous ammonia (68.72 mg, 4.04 mmol) was vigorously bubbled through it at 25° C. for 1 hr. The resulting mixture was filtered to remove ammonium chloride, the filtercake was washed with THF (2*20 ml) and discarded. The combined filtrate was concentrated in vacuo to afford crude N′-benzyl-N′-(4-pyridylmethyl)oxamide (0.9 g, 3.34 mmol, 82.83% yield) as red solid, which was used directly in the next step.

LCMS (ESI): [M+H]⁺m/z: calcd 270.1; found 270.0; Rt=0.747 min.

### Step 3: N-[4-amino-2-(2-trimethylsilylethoxymethyl)pyrazolo[4,3-c]pyridin-7-yl]-N′-benzyl-N′-(4-pyridylmethyl)oxamide

A mixture of N′-benzyl-N′-(4-pyridylmethyl)oxamide (300 mg, 1.11 mmol), 7-bromo-2-(2-trimethylsilylethoxymethyl)pyrazolo[4,3-c]pyridin-4-amine (300 mg, 873.89 μmol), copper (5 mg, 78.68 μmol), Copper (I) iodide (100 mg, 525.07 μmol, 17.79 μL), cesium carbonate (725.93 mg, 2.23 mmol) and rac-(1R,2R)—N1,N2-dimethylcyclohexane-1,2-diamine (100 mg, 703.04 μmol) in 1,4-dioxane (6 mL) was stirred in a sealed vial under argon at 105° C. for 60 hr. The resulting mixture was cooled down and submitted to reverse phase HPLC (column: XBridge C18 100×19 mm, 5 μm; mobile phase: 40-90% 0-5 min H₂O/MeOH/0.1% NH₄OH, flow rate: 30 ml/min (loading pump 4 ml/min acetonitrile) to afford N-[4-amino-2-(2-trimethylsilylethoxymethyl)pyrazolo[4,3-c]pyridin-7-yl]-N′-benzyl-N′-(4-pyridylmethyl)oxamide (62 mg, 116.61 μmol, 10.47% yield) as a light-brown solid.

LCMS (ESI): [M+H]⁺m/z: calcd 532.3; found 532.2; Rt=0.923 min.

### Step 4: The synthesis of N-(4-amino-1H-pyrazolo[4,3-c]pyridin-7-yl)-N′-benzyl-N′-(pyrimidin-2-ylmethyl)oxamide

Hydrogen chloride solution 4.0M in dioxane (3.15 g, 12.01 mmol, 3 mL, 13.9% purity) was added to a stirred solution of N-[4-amino-2-(2-trimethylsilylethoxymethyl)pyrazolo[4,3-c]pyridin-7-yl]-N′-benzyl-N′-(4-pyridylmethyl)oxamide (62 mg, 116.61 μmol) in methanol (3 mL) at 25° C. The resulting solution was stirred at 25° C. for 15 hr, then concentrated to dryness in vacuo and the residue was submitted to reverse phase HPLC (column: Chromatorex 18 SMB100-5T 100×19 mm 5 μm; mobile phase: May 5, 2025% 0-1-5 min H₂O/MeOH/0.1% FA, flow: 30 ml/min (loading pump 4 ml/min acetonitrile)) to afford Compound 27 N-(4-amino-1H-pyrazolo[4,3-c]pyridin-7-yl)-N′-benzyl-N′-(4-pyridylmethyl)oxamide (20.7 mg, 46.26 μmol, 39.67% yield, HCOOH) as a light-yellow solid.

¹H NMR (600 MHz, dmso) 8 4.45-4.58 (m, 2H), 4.67-4.87 (m, 2H), 7.15-7.37 (m, 9H), 7.61-7.79 (m, 1H), 8.12 (s, 1H), 8.25-8.42 (m, 1H), 8.49-8.57 (m, 2H), 9.83-10.88 (m, 1H), 12.75-13.72 (m, 1H).

LCMS (ESI): [M+1]⁺m/z: calcd 402.2; found 402.2; Rt=1.305 min.

### Example 4. Synthesis of N-(4-amino-1H-pyrazolo[4,3-c]pyridin-7-yl)-N′-benzyl-N′-(pyrimidin-2-ylmethyl)oxamide (Compound 38)

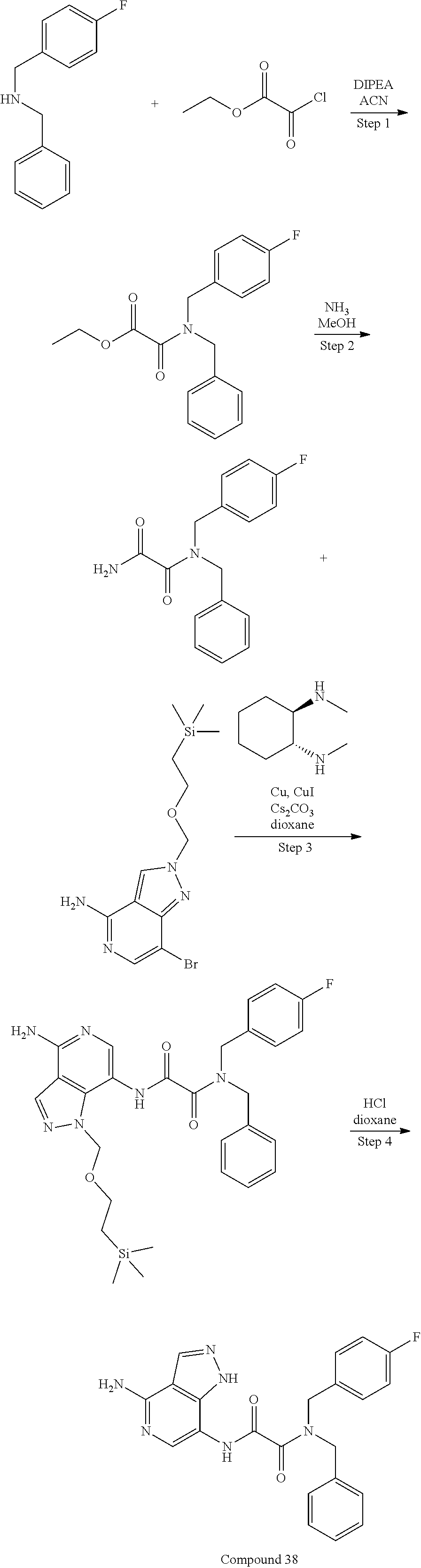

### Step 1: ethyl 2-[benzyl-[(4-fluorophenyl)methyl]amino]-2-oxo-acetate

ethyl 2-chloro-2-oxo-acetate (697.68 mg, 5.11 mmol, 570.93 μL) was added dropwise to an ice bath cold stirred solution of N-[(4-fluorophenyl)methyl]-1-phenyl-methanamine (1 g, 4.65 mmol) and DIPEA (780.49 mg, 6.04 mmol, 1.05 mL) in acetonitrile (14.71 mL). The reaction mixture was stirred overnight and concentrated on rotary evaporator. The residue was taken up in DCM and washed with water. The organic layer was separated, dried over Na₂SO₄ and concentrated under reduced pressure to afford ethyl 2-[benzyl-[(4-fluorophenyl)methyl]amino]-2-oxo-acetate (1.1 g, 3.49 mmol, 75.09% yield) which was used in the next step without purification.

LCMS (ESI): [M+H]⁺m/z: calcd 316.1; found 316.0; Rt=1.422 min.

### Step 2: N′-benzyl-N′-[(4-fluorophenyl)methyl]oxamide

ethyl 2-[benzyl-[(4-fluorophenyl)methyl]amino]-2-oxo-acetate (1.1 g, 3.49 mmol) was dissolved in methanol/NH₃ solution and stirred overnight. Upon completion, the reaction mixture was concentrated under reduced pressure to afford N′-benzyl-N′-[(4-fluorophenyl)methyl]oxamide (0.8 g, crude) which was used in the next step without purification.

LCMS (ESI): [M+H]⁺m/z: calcd 287.1; found 287.0; Rt=1.147 min.

### Step 3: N-[4-amino-1-(2-trimethylsilylethoxymethyl)pyrazolo[4,3-c]pyridin-7-yl]-N′-benzyl-N′-[(4-fluorophenyl)methyl]oxamide

N′-benzyl-N′-[(4-fluorophenyl)methyl]oxamide (0.3 g, 1.05 mmol), 7-bromo-1-(2-trimethylsilylethoxymethyl)pyrazolo[4,3-c]pyridin-4-amine (359.72 mg, 1.05 mmol), copper (0.05 g, 786.78 μmol), Copper (I) iodide (0.12 g, 630.09 μmol, 21.35 μL), (1S,2S)-N,N′-Bis -methyl-1,2-cyclohexane-diamine (149.05 mg, 1.05 mmol, 165.24 μL) were mixed in dioxane (6 mL). The reaction mixture was stirred under Ar atmosphere at 100° C. for 48 hr. Upon completion, the mixture was filtered and filtrate was concentrated under reduced pressure. The residue was purified by HPLC (0.6-6.5 min50-75% ACN+FA, 30 ml/min (loading pump 4 ml ACN) column: SunFire 100*19 mm, 5 microM) to afford N-[4-amino-1-(2-trimethylsilylethoxymethyl)pyrazolo[4,3-c]pyridin-7-yl]-N′-benzyl-N′-[(4-fluorophenyl)methyl]oxamide (0.05 g, crude).

LCMS (ESI): [M+H]⁺m/z: calcd 549.3; found 549.2; Rt=1.367 min.

### Step 4: The synthesis of N-(4-amino-1H-pyrazolo[4,3-c]pyridin-7-yl)-N′-benzyl-N′-[(4-fluorophenyl)methyl]oxamide

N-[4-amino-1-(2-trimethylsilylethoxymethyl)pyrazolo[4,3-c]pyridin-7-yl]-N′-benzyl-N′-[(4-fluorophenyl)methyl]oxamide (0.05 g, 91.13 μmol) was dissolved in HCl/dioxane solution (12%, 3 ml) and stirred at RT for 2 hr. Upon completion, the reaction mixture was concentrated under reduced pressure and the residue was submitted to reverse phase HPLC (2-10 min 0-70% acn+FA 30 ml/min) to afford N-(4-amino-1H-pyrazolo[4,3-c]pyridin-7-yl)-N′-benzyl-N′-[(4-fluorophenyl)methyl]oxamide (0.013 g, 31.07 μmol, 34.09% yield).

¹H NMR (600 MHz, dmso) δ 4.29-4.48 (m, 2H), 4.49-4.75 (m, 2H), 6.81-7.14 (m, 2H), 7.14-7.19 (m, 2H), 7.24-7.31 (m, 3H), 7.32-7.37 (m, 3H), 7.37-7.43 (m, 1H), 7.55-7.81 (m, 1H), 8.18-8.40 (m, 1H), 9.73-10.83 (m, 1H), 12.68-13.53 (m, 1H).

LCMS (ESI): [M+1]⁺m/z: calcd 419.2; found 419.2; Rt=2.067 min.

### Example 5. Synthesis of N1-(4-amino-1H-pyrazolo[4,3-c]pyridin-7-yl)-N2-((1R,2R)-2-(difluoromethoxy)cyclopentyl)-N2-((5-(trifluoromethyl)pyridin-2-yl)methyl)oxalamide (Compound 43)

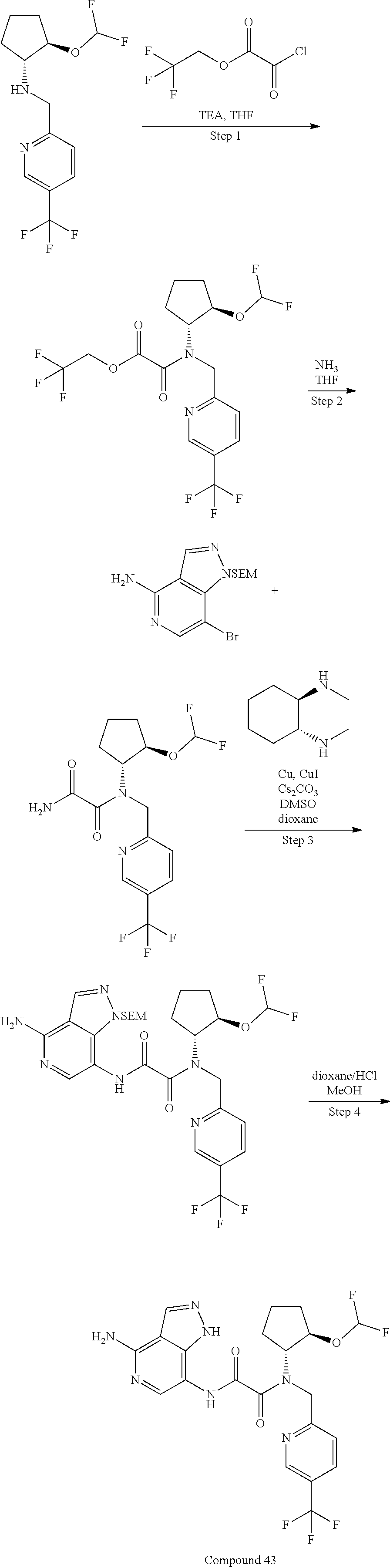

The synthesis of (1R,2R)-2-(difluoromethoxy)-N-((5-(trifluoromethyl)pyridin-2-yl)methyl)cyclopentan-1-amine is described in Intermediate 3.

### Step 1:2,2,2-trifluoroethyl 2-(((1R,2R)-2-(difluoromethoxy)cyclopentyl) ((5-(trifluoromethyl)pyridin-2-yl)methyl)amino)-2-oxoacetate

2,2,2-trifluoroethyl 2-chloro-2-oxo-acetate (429.81 mg, 2.26 mmol) was added dropwise to a stirred solution of (1S,2S)-2-(difluoromethoxy)-N-[[5-(trifluoromethyl)-2-pyridyl]methyl]cyclopentanamine (0.5 g, 1.61 mmol) and TEA (260.91 mg, 2.58 mmol, 359.39 μL) in THF (9.64 mL) at 0° C., stirred for 1 hr at 0° C. and 2 hr at 25° C. Reaction mixture was concentrated, triturated with water (20 mL), filtered, washed with TBME (10 mL), dried on air at 50° C. to give 2,2,2-trifluoroethyl 2-(((1R,2R)-2-(difluoromethoxy)cyclopentyl) ((5-(trifluoromethyl)pyridin-2-yl)methyl)amino)-2-oxoacetate (0.745 g, 1.60 mmol, 99.57% yield).

LCMS (ESI): [M+H]⁺m/z: calcd 465.1; found 465.4; Rt=4.313 min.

### Step 2: N1-((1R,2R)-2-(difluoromethoxy)cyclopentyl)-N1-((5-(trifluoromethyl)pyridin-2-yl)methyl)oxalamide

Ammonium was bubbled trough a suspension of 2,2,2-trifluoroethyl 2-oxo-2-[(1S,2S)-2-(difluoromethoxy)cyclopentyl]-[5-(trifluoromethyl)-2-pyridyl]methyl]amino]acetate (0.745 g, 1.60 mmol)(RM from previous step) at 0° C. for 0.25 hr and stirred for 1 hr additional at the same temperature. Reaction mixture was filtered, solid washed with THE (10 mL), combined filtrated concentrated to give pure N¹-((1R,2R)-2-(difluoromethoxy)cyclopentyl)-N1-((5-(trifluoromethyl)pyridin-2-yl)methyl)oxalamide (0.6 g, 1.57 mmol, 98.07% yield).

LCMS (ESI): [M+H]⁺m/z: calcd 382.1; found 382.0; Rt=1.241 min.

### Step 3: N1-(4-amino-1-((2-(trimethylsilyl) ethoxy)methyl)-1H-pyrazolo[4,3-c]pyridin-7-yl)-N2-((1R,2R)-2-(difluoromethoxy)cyclopentyl)-N2-((5-(trifluoromethyl)pyridin-2-yl)methyl)oxalamide

Copper (21.33 mg, 335.70 μmol), Copper (I) iodide (63.93 mg, 335.70 μmol, 11.38 μL), cesium carbonate (820.32 mg, 2.52 mmol) was added to a stirred solution of N′—[(1S,2S)-2-(difluoromethoxy)cyclopentyl]-N′-[5-(trifluoromethyl)-2-pyridyl]methyl]oxamide (0.32 g, 839.24 μmol), 7-bromo-1-(2-trimethylsilylethoxymethyl)pyrazolo[4,3-c]pyridin-4-amine (316.92 mg, 923.16 μmol), rac-(1R,2R)—N1,N2-dimethylcyclohexane-1,2-diamine (232.78 mg, 1.64 mmol) in DMSO(999.76 μL)/1,4-dioxane (5 mL) under Ar atmosphere and stirred at 90° C. for 48 hr. Reaction mixture was filtered, solid washed with dioxane (2×2 mL), filtrate concentrated to give crude N1-(4-amino-1-((2-(trimethylsilyl) ethoxy)methyl)-1H-pyrazolo[4,3-c]pyridin-7-yl)-N2-((1R,2R)-2-(difluoromethoxy)cyclopentyl)-N2-((5-(trifluoromethyl)pyridin-2-yl)methyl)oxalamide (0.54 g, 838.93 μmol, 99.96% yield).

LCMS (ESI): [M+H]⁺m/z: calcd 644.3; found 644.4; Rt=3.728 min.

### Step 4: The synthesis of N1-(4-amino-1H-pyrazolo[4,3-c]pyridin-7-yl)-N2-((1R,2R)-2-(difluoromethoxy)cyclopentyl)-N2-((5-(trifluoromethyl)pyridin-2-yl)methyl)oxalamide

Hydrogen chloride solution 4.0M in dioxane (800.00 mg, 21.94 mmol, 1 mL) was added to a stirred solution of N-[4-amino-1-(2-trimethylsilylethoxymethyl)pyrazolo[4,3-c]pyridin-7-yl]-N′—[(1S,2S)-2-(difluoromethoxy)cyclopentyl]-N′-[5-(trifluoromethyl)-2-pyridyl]methyl]oxamide (0.54 g, 838.93 μmol) in Methanol (1 mL) and stirred at 20° C. for 12 hr. Reaction mixture was concentrated and purified by HPLC (column: YMC Triart C18 100×20 mm, 5 μm; mobile phase: 20-45% 0-5 min H₂O/ACN/0.1% NH₄OH, flow rate: 30 ml/min) to give crude N-(4-amino-1H-pyrazolo[4,3-c]pyridin-7-yl)-N′—[(1S,2S)-2-(difluoromethoxy)cyclopentyl]-N′-[5-(trifluoromethyl)-2-pyridyl]methyl]oxamide (44 mg, 85.70 μmol, 10.22% yield). 1 fraction after first purification was purified by HPLC (column: YMC Triart C18 100×20 mm, 5 μm; mobile phase: 10-50% 0-5 min H₂O/ACN/0.1% NH₄OH, flow rate: 30 ml/min) to give pure N1-(4-amino-1H-pyrazolo[4,3-c]pyridin-7-yl)-N2-((1R,2R)-2-(difluoromethoxy)cyclopentyl)-N2-((5-(trifluoromethyl)pyridin-2-yl)methyl)oxalamide (44 mg, 85.70 μmol, 10.22% yield) as a yellow solid.

¹H NMR (DMSO-d6, 600 MHz): δ (ppm) 1.69 (m, 4H), 1.95 (m, 2H), 4.38 (m, 1H), 4.71 (m, 1H), 4.81 (m, 1H), 5.11 (m, 1H), 6.58 (m, 3H), 7.53 (m, 1H), 7.69 (m, 1H), 8.16 (m, 2H), 8.89 (m, 1H), 10.42 (m, 1H), 12.68 (m, 1H).

LCMS (ESI): [M+H]⁺m/z: calcd 514.2; found 514.0; Rt=1.961 min.

### Example 6. Synthesis of N-(4-amino-1H-pyrazolo[4,3-c]pyridin-7-yl)-N′-(1-pyrimidin-2-ylethyl)-N′-[[5-(trifluoromethyl)-2-pyridyl]methyl]oxamide (Compound 50)

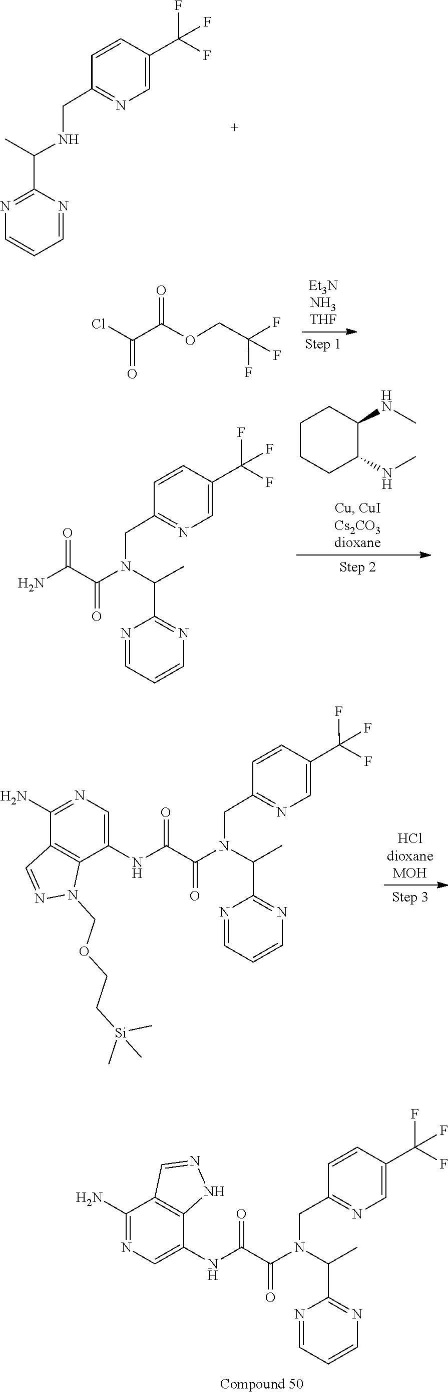

### Step 1: N′-(1-pyrimidin-2-ylethyl)-N′-[[5-(trifluoromethyl)-2-pyridyl]methyl]oxamide)

2,2,2-trifluoroethyl 2-chloro-2-oxo-acetate (877.39 mg, 4.61 mmol) was added slowly to a cooled to −10° C. mixture of 1-pyrimidin-2-yl-N-[[5-(trifluoromethyl)-2-pyridyl]methyl]ethanamine (1 g, 3.54 mmol) and triethyl amine (2.15 g, 21.26 mmol, 2.96 mL) in THF (60 mL). The resulting mixture was allowed to warm to 25° C. and stirred for 2 hr. Then gaseous ammonia (60.33 mg, 3.54 mmol) was vigorously bubbled through it at 25° C. for 1 hr. The resulting mixture was filtered to remove ammonium chloride, the filtercake was washed with THF (2*20 ml) and discarded. The combined filtrate was concentrated in vacuo to afford crude N′-(1-pyrimidin-2-ylethyl)-N′-[[5-(trifluoromethyl)-2-pyridyl]methyl]oxamide (1.4 g, crude) as red solid, which was used directly in the next step.

LCMS (ESI): [M+H]⁺m/z: calcd 354.1; found 354.0; Rt=0.977 min.

### Step 2: N-[4-amino-1-(2-trimethylsilylethoxymethyl)pyrazolo[4,3-c]pyridin-7-yl]-N′-(1-pyrimidin-2-ylethyl)-N′-[[5-(trifluoromethyl)-2-pyridyl]methyl]oxamide

A mixture of N′-(1-pyrimidin-2-ylethyl)-N′-[[5-(trifluoromethyl)-2-pyridyl]methyl]oxamide (300 mg, 849.14 μmol), 7-bromo-1-(2-trimethylsilylethoxymethyl)pyrazolo[4,3-c]pyridin-4-amine (336.00 mg, 978.75 μmol), copper (5.00 mg, 78.68 μmol), Copper (I) iodide (120.00 mg, 630.09 μmol, 21.35 μL), cesium carbonate (608.67 mg, 1.87 mmol) and rac-(1R,2R)—N1,N2-dimethylcyclohexane-1,2-diamine (132.00 mg, 928.01 μmol) in 1,4-dioxane (6.00 mL) was stirred in a sealed vial under argon at 105° C. for 48 hr. The resulting mixture was cooled down and submitted to reverse phase HPLC (column: XBridge C18 100×19 mm, 5 μm; mobile phase: 40-90% 0-5 min H₂O/MeOH/0.1% NH₄OH, flow rate: 30 ml/min (loading pump 4 ml/min methanol) to afford N-[4-amino-1-(2-trimethylsilylethoxymethyl)pyrazolo[4,3-c]pyridin-7-yl]-N′-(1-pyrimidin-2-ylethyl)-N′-[[5-(trifluoromethyl)-2-pyridyl]methyl]oxamide (136 mg, 220.89 μmol, 26.01% yield) as a light-brown solid.

LCMS (ESI): [M-t-Bu+1]⁺m/z: calcd 616.3; found 616.2; Rt=1.258 min.

### Step 3: The synthesis of N-(4-amino-1H-pyrazolo[4,3-c]pyridin-7-yl)-N′-(1-pyrimidin-2-ylethyl)-N′-[[5-(trifluoromethyl)-2-pyridyl]methyl]oxamide (Compound 50)

Hydrogen chloride solution 4.0M in dioxane (3.15 g, 12.01 mmol, 3 mL, 13.9% purity) was added to a stirred solution of N-[4-amino-1-(2-trimethylsilylethoxymethyl)pyrazolo[4,3-c]pyridin-7-yl]-N′-(1-pyrimidin-2-ylethyl)-N′-[5-(trifluoromethyl)-2-pyridyl]methyl]oxamide (131 mg, 212.77 μmol) in methanol (3 mL) at 25° C. The resulting mixture was stirred in a sealed vial at 25° C. for 15 hr, and then concentrated to dryness in vacuo. The residue was submitted to reverse phase HPLC (Chromatorex 18 SMB100-5T 100×19 mm 5 μm; mobile phase: May 5, 2030% 0-1-5 min H₂O/ACN/0.1% FA; flow rate: 30 ml/min (loading pump 4 ml/min acetonitrile) to afford Compound 50 N-(4-amino-1H-pyrazolo[4,3-c]pyridin-7-yl)-N′-(1-pyrimidin-2-ylethyl)-N′-[[5-(trifluoromethyl)-2-pyridyl]methyl]oxamide (90 mg, 169.35 μmol, 79.59% yield, HCOOH) as a light-yellow solid.

¹H NMR (DMSO-d6, 600 MHz): δ (ppm) 1.52-1.66 (d, 3H), 4.56-5.32 (m, 2H), 5.67-6.06 (q, 1H), 6.67-7.03 (m, 2H), 7.34-7.40 (m, 1H), 7.50-7.70 (m, 2H), 8.05-8.19 (m, 2H), 8.66-8.82 (m, 3H), 9.80 (m, 1H), 10.40-10.52 (m, 1H).

LCMS (ESI): [M+1]⁺m/z: calcd 486.2; found 486.2; Rt=1.945 min.

### Example 7. Synthesis of N-(4-amino-1H-pyrazolo[4,3-c]pyridin-7-yl)-N′,N′-dibenzyl-oxamide (Compound 21)

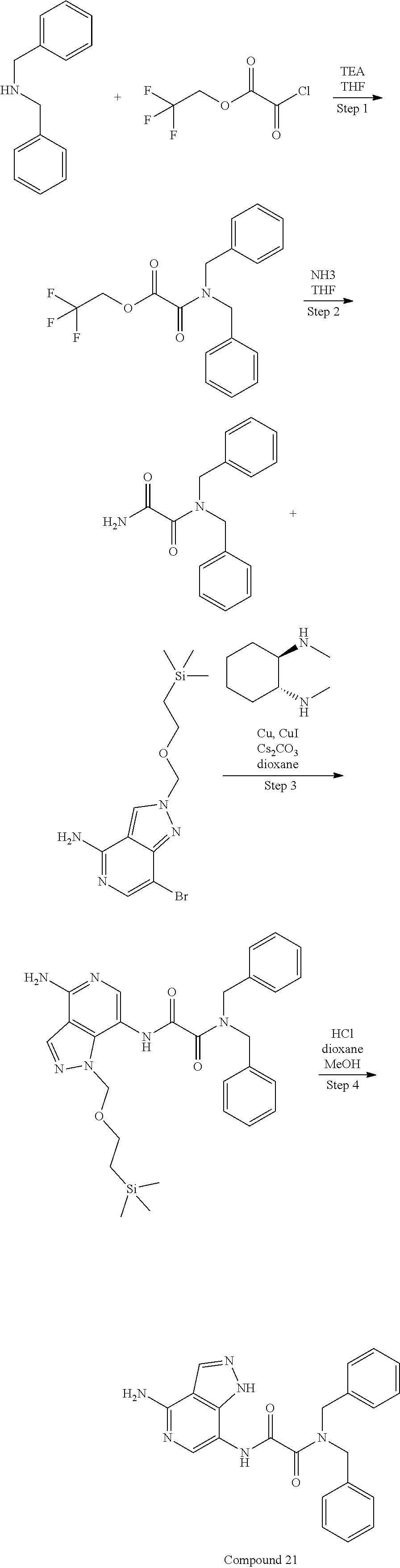

### Step 1:2,2,2-trifluoroethyl 2-(dibenzylamino)-2-oxo-acetate

To a solution of N-benzyl-1-phenyl-methanamine (1.5 g, 7.60 mmol, 1.46 mL) and TEA (1.15 g, 11.41 mmol, 1.59 mL) in THF (15 mL) was added 2,2,2-trifluoroethyl 2-chloro-2-oxo-acetate (1.74 g, 9.12 mmol) dropwise at 0° C. under argon. The reaction mixture was then stirred for 6 hr at r.t., then evaporated in vacuo to give 2,2,2-trifluoroethyl 2-(dibenzylamino)-2-oxo-acetate (1.6 g, 4.55 mmol, 59.90% yield).

LCMS (ESI): [M+H]⁺m/z: calcd 352.3; found 352.2; Rt=1.563 min.

### Step 2: N′,N′-dibenzyloxamide

2,2,2-trifluoroethyl 2-(dibenzylamino)-2-oxo-acetate (1.6 g, 4.55 mmol) was dissolved in THF (15 mL) and was blow ammonium (1.64 g, 91.09 mmol). Resulting solution was stirred at 0° C. for 5 hr. The resulting mixture was evaporated in vacuo and residue was triturated with THF (20 ml*2), filtered and combined organic was evaporated in vacuo to afford N′,N′-dibenzyloxamide (1.3 g, crude).

LCMS (ESI): [M+H]⁺m/z: calcd 270.1; found 270.0; Rt=0.747 min.

### Step 3: N-[4-amino-1-(2-trimethylsilylethoxymethyl)pyrazolo[4,3-c]pyridin-7-yl]-N′,N′-dibenzyl-oxamide

7-bromo-1-(2-trimethylsilylethoxymethyl)pyrazolo[4,3-c]pyridin-4-amine (460.61 mg, 1.34 mmol), N′,N′-dibenzyloxamide (0.3 g, 1.12 mmol), Cu(3.55 mg, 55.91 μmol), CuI (42.59 mg, 223.62 μmol, 7.58 μL), Potassium carbonate (309.07 mg, 2.24 mmol, 134.96 μL) and (1R,2R)—N1,N2-dimethylcyclohexane-1,2-diamine (15.90 mg, 111.81 μmol) were mixed in dioxane (4 mL), purged with Ar for 15 minutes and then heated in the sealed tube at 100° C. for 48 hr. Final mixture was filtered and dioxane was evaporated in vacuo. The crude product 0.4 g was purified by RP-HPLC (column: XBridge BEH C18 5 μm 130A; 65-65-80% 0-1-6 min H₂O/CH₃OH/0.1% NH₄OH, flow: 30 ml/min) to give N-[4-amino-1-(2-trimethylsilylethoxymethyl)pyrazolo[4,3-c]pyridin-7-yl]-N′,N′-dibenzyl-oxamide (78.60 mg, 148.11 μmol, 13.25% yield).

LCMS (ESI): [M+H]⁺m/z: calcd 531.3; found 531.0; Rt=1.416 min.

### Step 4: The synthesis of N-(4-amino-1H-pyrazolo[4,3-c]pyridin-7-yl)-N′,N′-dibenzyl-oxamide

Hydrogen chloride solution 4.0M in dioxane (800.00 mg, 21.94 mmol, 1 mL) was added to a solution of N-[4-amino-1-(2-trimethylsilylethoxymethyl)pyrazolo[4,3-c]pyridin-7-yl]-N′,N′-dibenzyl-oxamide (0.048 g, 90.45 μmol) in MeOH (2 mL). The reaction mixture was stirred at 20° C. for 48 hr, then evaporated was purified by RP-HPLC (column: Chromatorex 18 SMB100-BT 100*19 mm; 10-60% 0-5 min H₂O/CH₃CN/0.1% FA, flow: 30 ml/min) to give N-(4-amino-1H-pyrazolo[4,3-c]pyridin-7-yl)-N′,N′-dibenzyl-oxamide (0.033 g, 67.01 μmol, 74.08% yield, 2HCOOH).

¹H NMR (600 MHz, dmso) δ 3.96-4.72 (m, 4H), 6.27-7.22 (m, 3H), 7.26-7.31 (m, 3H), 7.31-8.24 (m, 8H), 9.79-10.69 (m, 1H), 12.66-13.43 (m, 1H).

LCMS (ESI): [M+H]⁺m/z: calcd 401.2; found 401.2; Rt=2.468 min.

### Example 8. Synthesis of N-(4-amino-1H-pyrazolo[4,3-c]pyridin-7-yl)-N′-benzyl-N′-(2-pyridylmethyl)oxamide (Compound 9)

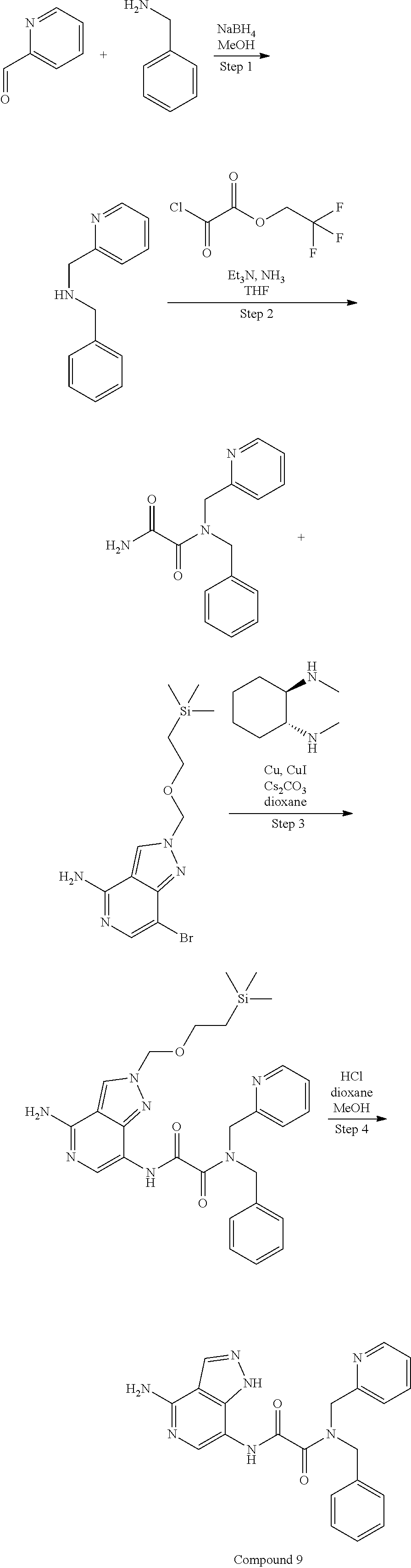

### Step 1:1-phenyl-N-(4-pyridylmethyl)methanamine

pyridine-2-carbaldehyde (1000.00 mg, 9.34 mmol, 889.68 μL) was added to a solution of phenylmethanamine (1.00 g, 9.34 mmol) in methanol (25 mL) at 25° C. The resulting mixture was stirred at 25° C. for 15 hr, then cooled to 0° C. and Sodium Borohydride (353.21 mg, 9.34 mmol) was added in one portion. The reaction mixture was allowed to warm to 25° C. and stirred for 1 hr, then concentrated in vacuo. The residue was diluted with water (25 ml) and extracted with dichloromethane (40 ml). The organic layer was separated, dried over sodium sulfate and concentrated in vacuo to afford crude 1-phenyl-N-(2-pyridylmethyl)methanamine (1.65 g, 8.32 mmol, 89.14% yield) as a yellow oil.

LCMS (ESI): [M+H]⁺m/z: calcd 199.2; found 199.2; Rt=0.568 min.

### Step 2: N′-benzyl-N′-(2-pyridylmethyl)oxamide

2,2,2-trifluoroethyl 2-chloro-2-oxo-acetate (1.2 g, 6.30 mmol) was added slowly to a cooled to -10° C. mixture of 1-phenyl-N-(2-pyridylmethyl)methanamine (800.00 mg, 4.04 mmol) and triethyl amine (2 g, 19.76 mmol, 2.75 mL) in THF (50 mL). The resulting mixture was allowed to warm to 25° C. and stirred for 2 hr. Then gaseous ammonia (68.72 mg, 4.04 mmol) was vigorously bubbled through it at 25° C. for 1 hr. The resulting mixture was filtered to remove ammonium chloride, the filtercake was washed with THF (2*20 ml) and discarded. The combined filtrate was concentrated in vacuo to afford crude N′-benzyl-N′-(2-pyridylmethyl)oxamide (1.1 g, crude) as red gum, which was used directly in the next step.

LCMS (ESI): [M+H]⁺m/z: calcd 270.1; found 270.0; Rt=0.924 min.

### Step 3: N-[4-amino-2-(2-trimethylsilylethoxymethyl)pyrazolo[4,3-c]pyridin-7-yl]-N′-benzyl-N′-(2-pyridylmethyl)oxamide

A mixture of N′-benzyl-N′-(2-pyridylmethyl)oxamide (300.00 mg, 1.11 mmol), 7-bromo-2-(2-trimethylsilylethoxymethyl)pyrazolo[4,3-c]pyridin-4-amine (300 mg, 873.89 μmol), copper (5 mg, 78.68 μmol), Copper (I) iodide (100 mg, 525.07 μmol, 17.79 μL), cesium carbonate (725.93 mg, 2.23 mmol) and rac-(1R,2R)—N1,N2-dimethylcyclohexane-1,2-diamine (100 mg, 703.04 μmol) in 1,4-dioxane (6 mL) was stirred in a sealed vial under argon at 105° C. for 60 hr. The resulting mixture was cooled down and submitted to reverse phase HPLC (column: XBridge C18 100×19 mm, 5 μm; mobile phase: 40-90% 0-5 min H₂O/MeOH/0.1% NH₄OH, flow rate: 30 ml/min (loading pump 4 ml/min acetonitrile) to afford two fractions of the N-[4-amino-2-(2-trimethylsilylethoxymethyl)pyrazolo[4,3-c]pyridin-7-yl]-N′-benzyl-N′-(2-pyridylmethyl)oxamide (117 mg, 220.06 μmol, 19.75% yield) as light-brown solids, which were combined and used in the next step (SEM-deprotection).

LCMS (ESI): [M+H]⁺m/z: calcd 532.3; found 532.2; Rt=0.937 min.

### Step 4: The synthesis of N-(4-amino-1H-pyrazolo[4,3-c]pyridin-7-yl)-N′-benzyl-N′-(2-pyridylmethyl)oxamide

Hydrogen chloride solution 4.0M in dioxane (3.68 g, 14.01 mmol, 3.5 mL, 13.9% purity) was added to a stirred solution of N-[4-amino-2-(2-trimethylsilylethoxymethyl)pyrazolo[4,3-c]pyridin-7-yl]-N′-benzyl-N′-(2-pyridylmethyl)oxamide (117 mg, 220.06 μmol) in methanol (3.5 mL) at 25° C. The resulting solution was stirred at 25° C. for 15 hr, then concentrated to dryness in vacuo and the residue was submitted to reverse phase HPLC (column: Chromatorex 18 SMB100-5T 100×19 mm 5 μm; mobile phase: May 5, 2025% 0-1-5 min H₂O/MeOH/0.1% FA, flow: 30 ml/min (loading pump 4 ml/min acetonitrile)) to afford Compound 9 N-(4-amino-1H-pyrazolo[4,3-c]pyridin-7-yl)-N′-benzyl-N′-(2-pyridylmethyl)oxamide (49 mg, 109.51 μmol, 49.76% yield, HCOOH) as a light-yellow solid.

¹H NMR (600 MHz, dmso) δ 4.29-4.61 (m, 2H), 4.82-4.93 (m, 2H), 6.34-6.99 (m, 2H), 7.27-7.41 (m, 7H), 7.56-7.68 (m, 1H), 7.72-7.81 (m, 1H), 8.11-8.21 (m, 2H), 8.29-8.59 (m, 1H), 9.63-10.65 (m, 1H), 12.73-13.61 (m, 1H).

LCMS (ESI): [M+1]⁺m/z: calcd 402.2; found 402.0; Rt=1.754 min.

### Example 9. Synthesis of (R)—N¹-(4-amino-1HI-pyrazolo[4,3-c]pyridin-7-yl)-N2-(1-cyclopropyl-2-methoxyethyl)-N2-((5-(trifluoromethyl)pyridin-2-yl)methyl)oxalamide (Compound 39)

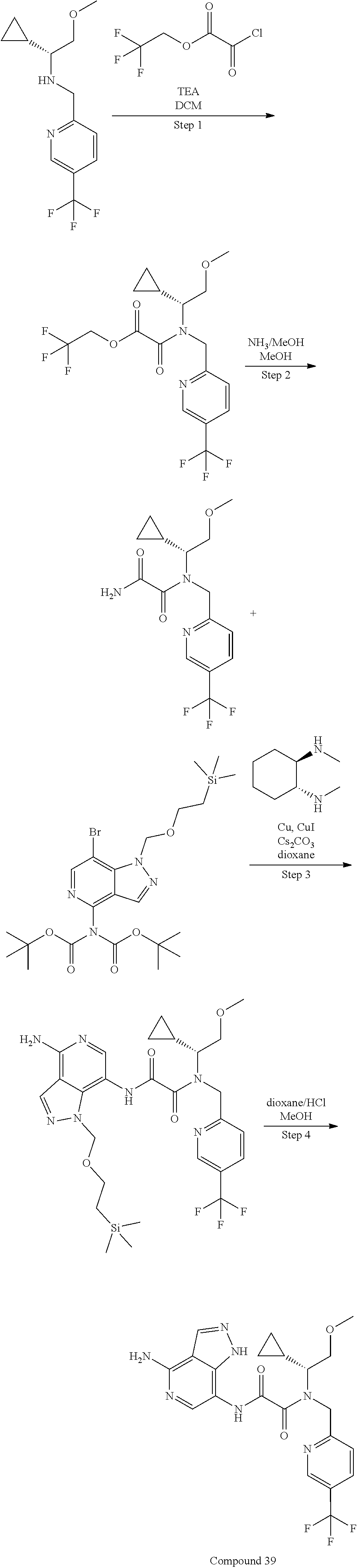

The synthesis of (R)-1-cyclopropyl-2-methoxy-N-((5-(trifluoromethyl)pyridin-2-yl)methyl) ethan-1-amine is described in Intermediate 1.

### Step 1: (R)-2,2,2-trifluoroethyl 2-((1-cyclopropyl-2-methoxyethyl)((5-(trifluoromethyl)pyridin-2-yl)methyl)amino)-2-oxoacetate

To a stirred solution of (1R)-1-cyclopropyl-2-methoxy-N-[[5-(trifluoromethyl)-2-pyridyl]methyl]ethanamine (0.2 g, 729.18 μmol) and Triethylamine (145.20 mg, 1.43 mmol, 0.2 mL) in CHCl₃ (5 mL) was added 2,2,2-trifluoroethyl 2-chloro-2-oxo-acetate (0.2 g, 1.05 mmol). The resulting reaction mixture was stirred at 25° C. for 16 hr. Upon completion, the reaction mixture was evaporated, quenched with water (20 mL). The aqueous phase was extracted with CHCl₃ (2×20 mL). The combined organic phase was dried over Na₂SO₄ and concentrated under reduced pressure. The desired product (R)-2,2,2-trifluoroethyl 2-((1-cyclopropyl-2-methoxyethyl)((5-(trifluoromethyl)pyridin-2-yl)methyl)amino)-2-oxoacetate (0.3 g, 700.40 μmol, 96.05% yield) was isolated.

LCMS (ESI): [M+H]⁺m/z: calcd 429.1; found 429.0; Rt=1.456 min.

### Step 2: (R)—N¹-(1-cyclopropyl-2-methoxyethyl)-N1-((5-(trifluoromethyl)pyridin-2-yl)methyl)oxalamide

To a stirred solution of 2,2,2-trifluoroethyl 2-oxo-2-[(1R)-1-cyclopropyl-2-methoxy-ethyl]-[[5-(trifluoromethyl)-2-pyridyl]methyl]amino]acetate (0.3 g, 700.40 μmol) in MeOH (3 mL) was added NH₃/MeOH (3 mL). The resulting reaction mixture was stirred at 25° C. for 16 hr. Upon completion, the reaction mixture was concentrated under reduced pressure. The desired product (R)—N¹-(1-cyclopropyl-2-methoxyethyl)-N1-((5-(trifluoromethyl)pyridin-2-yl)methyl)oxalamide (0.24 g, 695.02 μmol, 99.23% yield) was isolated.

LCMS (ESI): [M+H]⁺m/z: calcd 346.1; found 346.2; Rt=1.148 min.

### Step 3: (R)—N¹-(4-amino-1-((2-(trimethylsilyl) ethoxy)methyl)-1H-pyrazolo[4,3-c]pyridin-7-yl)-N2-(1-cyclopropyl-2-methoxyethyl)-N2-((5-(trifluoromethyl)pyridin-2-yl)methyl)oxalamide

N′—[(1R)-1-cyclopropyl-2-methoxy-ethyl]-N′-[5-(trifluoromethyl)-2-pyridyl]methyl]oxamide (0.15 g, 434.38 μmol), tert-butyl N-[7-bromo-1-(2-trimethylsilylethoxymethyl)pyrazolo[4,3-c]pyridin-4-yl]-N-tert-butoxycarbonyl-carbamate (0.25 g, 459.96 μmol), Copper (I) iodide (80 mg, 420.06 μmol, 14.23 μL), Cu(20 mg, 314.71 μmol) and Cesium carbonate (0.2 g, 613.84 μmol) were mixed together in DiOX (5 mL). The resulting suspension was degassed with argon at 25° C. for 0.1 hr. (1R,2R)—N1,N2-dimethylcyclohexane-1,2-diamine (158.00 mg, 1.11 mmol, 0.2 mL) was added thereto and the resulting mixture was stirred for 16 hr at 100° C. After completion the reaction mixture was filtered and the filtrate was concentrated in vacuum. The obtained crude product was purified by reverse phase HPLC chromatography (Device (Mobile Phase, Column): SYSTEM 40-90% 0-5 min H₂O/ACN/0.1% NH₄OH, flow: 30 ml/min (loading pump 4 ml/min methanol) target mass 607.71 column: XBridge BEH C18 100×19 mm, 5 μm) to afford (R)—N¹-(4-amino-1-((2-(trimethylsilyl) ethoxy)methyl)-1H-pyrazolo[4,3-c]pyridin-7-yl)-N2-(1-cyclopropyl-2-methoxyethyl)-N2-((5-(trifluoromethyl)pyridin-2-yl)methyl)oxalamide (59 mg, 97.09 μmol, 22.35% yield).

LCMS (ESI): [M+H]⁺m/z: calcd 608.3; found 608.2; Rt=1.230 min.

### Step 4: The synthesis of (R)—N¹-(4-amino-1H-pyrazolo[4,3-c]pyridin-7-yl)-N2-(1-cyclopropyl-2-methoxyethyl)-N2-((5-(trifluoromethyl)pyridin-2-yl)methyl)oxalamide

To a stirred solution of N-[4-amino-1-(2-trimethylsilylethoxymethyl)pyrazolo[4,3-c]pyridin-7-yl]-N′—[(1R)-1-cyclopropyl-2-methoxy-ethyl]-N′-[5-(trifluoromethyl)-2-pyridyl]methyl]oxamide (60 mg, 98.73 μmol) in MeOH (3 mL) was added DiOX/HCl (3 mL). The resulting reaction mixture was stirred at 25° C. for 2 hr. Upon completion, the reaction mixture was concentrated under reduced pressure. The obtained crude product was purified by reverse phase HPLC chromatography (Device (Mobile Phase, Column): SYSTEM 5-55% 0-5 min H₂O/ACN/0.1% FA, flow: 30 ml/min (loading pump 4 ml/min acetonitrile) target mass 477.45 column: Chromatorex 18 SMB100-5T 100×19 mm 5 μm) to afford (R)—N¹-(4-amino-1H-pyrazolo[4,3-c]pyridin-7-yl)-N2-(1-cyclopropyl-2-methoxyethyl)-N2-((5-(trifluoromethyl)pyridin-2-yl)methyl)oxalamide (10 mg, 19.10 μmol, 19.35% yield, HCOOH) as a white solid.

¹H NMR (600 MHz, dmso) 8 0.09-0.23 (m, 1H), 0.31-0.39 (m, 1H), 0.39-0.61 (m, 2H), 0.92-1.01 (m, 1H), 2.86-3.08 (m, 1H), 3.09-3.16 (m, 3H), 3.45-3.50 (m, 1H), 3.53-3.63 (m, 1H), 3.73-3.84 (m, 1H), 4.25-5.22 (m, 2H), 6.60-7.03 (m, 2H), 7.51-7.82 (m, 2H), 8.12-8.24 (m, 2H), 8.72-8.94 (m, 1H), 10.22-10.53 (m, 1H), 12.56-12.86 (m, 1H).

LCMS (ESI): [M+H]⁺m/z: calcd 478.2; found 478.2; Rt=2.562 min.

### Example 10. Synthesis of N-(4-amino-1H-pyrazolo[4,3-c]pyridin-7-yl)-N′-benzyl-N′-(3-pyridylmethyl)oxamide (Compound 29)

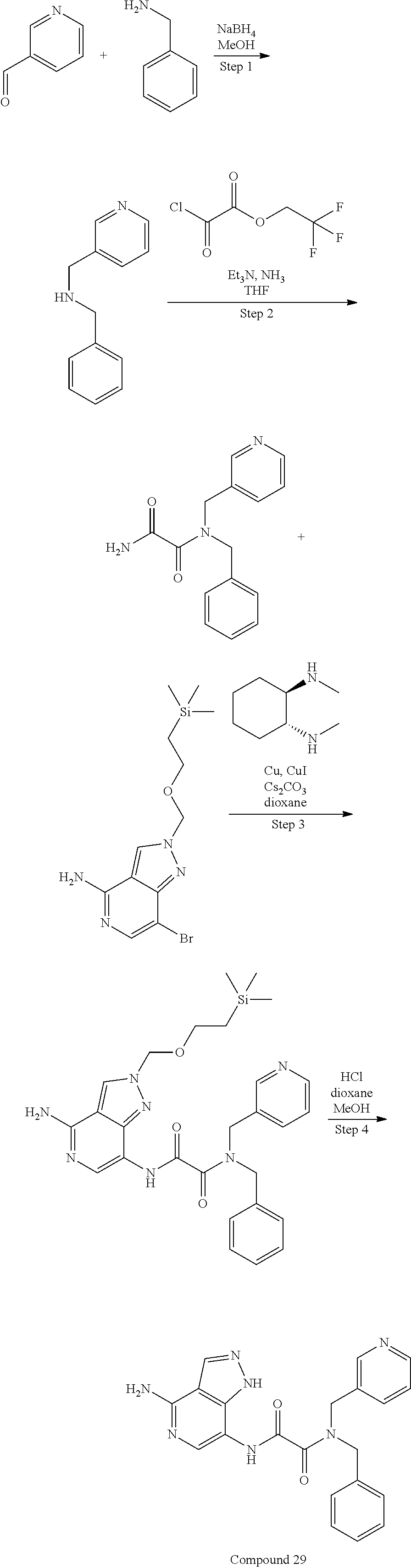

### Step 1:1-phenyl-N-(3-pyridylmethyl)methanamine

pyridine-3-carbaldehyde (1000.00 mg, 9.34 mmol, 876.42 μL) was added to a solution of phenylmethanamine (1.00 g, 9.34 mmol) in methanol (25 mL) at 25° C. The resulting mixture was stirred at 25° C. for 15 hr, then cooled to 0° C. and Sodium Borohydride (353.21 mg, 9.34 mmol) was added in one portion. The reaction mixture was allowed to warm to 25° C. and stirred for 1 hr, then concentrated in vacuo. The residue was diluted with water (25 ml) and extracted with dichloromethane (40 ml). The organic layer was separated, dried over sodium sulfate and concentrated in vacuo to afford 1-phenyl-N-(3-pyridylmethyl)methanamine (1.7 g, 8.57 mmol, 91.84% yield) as a yellow oil.

LCMS (ESI): [M+H]⁺m/z: calcd 199.1; found 199.2; Rt=0.258 min.

### Step 2: N′-benzyl-N′-(3-pyridylmethyl)oxamide

2,2,2-trifluoroethyl 2-chloro-2-oxo-acetate (1.2 g, 6.30 mmol) was added slowly to a cooled to −10° C. mixture of 1-phenyl-N-(3-pyridylmethyl)methanamine (800.00 mg, 4.04 mmol) and triethyl amine (2 g, 19.76 mmol, 2.75 mL) in THF (50 mL). The resulting mixture was allowed to warm to 25° C. and stirred for 2 hr. Then gaseous ammonia (68.72 mg, 4.04 mmol) was vigorously bubbled through it at 25° C. for 1 hr. The resulting mixture was filtered to remove ammonium chloride; the filtercake was washed with THF (2*20 ml) and discarded. The combined filtrate was concentrated in vacuo to afford crude N′-benzyl-N′-(3-pyridylmethyl)oxamide (1.1 g, crude) as red gum, which was used directly in the next step.

LCMS (ESI): [M+H]⁺m/z: calcd 270.1; found 270.2; Rt=0.719 min.

### Step 3: N-[4-amino-2-(2-trimethylsilylethoxymethyl)pyrazolo[4,3-c]pyridin-7-yl]-N′-benzyl-N′-(3-pyridylmethyl)oxamide

A mixture of N′-benzyl-N′-(3-pyridylmethyl)oxamide (300.00 mg, 1.11 mmol), 7-bromo-2-(2-trimethylsilylethoxymethyl)pyrazolo[4,3-c]pyridin-4-amine (300 mg, 873.89 μmol), copper (5 mg, 78.68 μmol), Copper (I) iodide (100 mg, 525.07 μmol, 17.79 μL), cesium carbonate (725.93 mg, 2.23 mmol) and rac-(1R,2R)—N1,N2-dimethylcyclohexane-1,2-diamine (100 mg, 703.04 μmol) in 1,4-dioxane (6 mL) was stirred in a sealed vial under argon at 105° C. for 60 hr. The resulting mixture was cooled down and submitted to reverse phase HPLC (column: XBridge BEH C18 100×19 mm, 5 μm; mobile phase: 40-90% 0-5 min H₂O/MeOH/0.1% NH₄OH, flow rate: 30 ml/min (loading pump 4 ml/min methanol) to afford N-[4-amino-2-(2-trimethylsilylethoxymethyl)pyrazolo[4,3-c]pyridin-7-yl]-N′-benzyl-N′-(3-pyridylmethyl)oxamide (96 mg, 180.56 μmol, 16.21% yield) as a light-brown solid.

LCMS (ESI): [M+H]⁺m/z: calcd 532.3; found 532.2; Rt=0.831 min.

### Step 4: The synthesis of N-(4-amino-1H-pyrazolo[4,3-c]pyridin-7-yl)-N′-benzyl-N′-(3-pyridylmethyl)oxamide

Hydrogen chloride solution 4.0M in dioxane (3.15 g, 12.01 mmol, 3 mL, 13.9% purity) was added to a stirred solution of N-[4-amino-2-(2-trimethylsilylethoxymethyl)pyrazolo[4,3-c]pyridin-7-yl]-N′-benzyl-N′-(3-pyridylmethyl)oxamide (96 mg, 180.56 μmol) in methanol (3 mL) at 25° C. The resulting solution was stirred at 25° C. for 15 hr, then concentrated to dryness in vacuo and the residue was submitted to reverse phase HPLC (column: Chromatorex 18 SMB100-5T 100×19 mm 5 μm; mobile phase: May 5, 2025% 0-1-5 min H₂O/MeOH/0.1% FA, flow: 30 ml/min (loading pump 4 ml/min acetonitrile)) to afford Compound 29 N-(4-amino-1H-pyrazolo[4,3-c]pyridin-7-yl)-N′-benzyl-N′-(3-pyridylmethyl)oxamide (43.7 mg, 97.67 μmol, 54.09% yield, HCOOH) as a light-yellow solid.

¹H NMR (600 MHz, dmso) 8 4.13-4.50 (m, 2H), 4.50-4.79 (m, 2H), 6.27-7.02 (m, 2H), 7.12-7.30 (m, 2H), 7.30-7.42 (m, 4H), 7.50-7.81 (m, 2H), 7.96-8.22 (m, 2H), 8.32-8.56 (m, 2H), 9.81-10.94 (m, 1H), 12.66-13.39 (m, 1H).

LCMS (ESI): [M+1]⁺m/z: calcd 402.2; found 402.0; Rt=1.403 min.

### Example 11. Synthesis of N-(4-amino-1H-pyrazolo[4,3-c]pyridin-7-yl)-N′-(pyrimidin-2-ylmethyl)-N′-[[5-(trifluoromethyl)-2-pyridyl]methyl]oxamide (Compound 25)

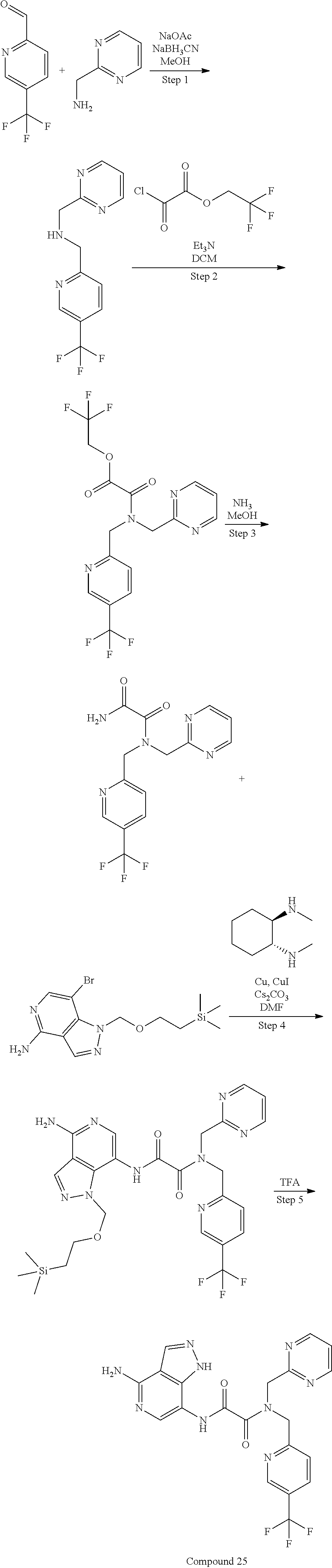

### Step 1: The synthesis of N-(pyrimidin-2-ylmethyl)-1-[5-(trifluoromethyl)-2-pyridyl]methanamine

pyrimidin-2-ylmethanamine (3.12 g, 21.40 mmol, HCl) was dissolved in MeOH (50 mL) and Sodium acetate, anhydrous (2.69 g, 32.84 mmol, 1.76 mL) was added thereto. The resulting mixture was stirred for 30 min and 5-(trifluoromethyl)pyridine-2-carbaldehyde (5 g, 28.55 mmol) was added followed by addition of Sodium cyanoborohydride (2.69 g, 42.83 mmol). The resulting mixture was stirred over weekend. The reaction mixture was concentrated in vacuo and aq·K₂CO₃ solution (50 ml) was added to the residue. The resulting mixture was extracted with DCM (2*50 ml) and combined organic layers were dried over Na₂SO₄, filtered and concentrated in vacuo. The residue was purified by column chromatography (gradient MeOH in MTBE from 0% to 100%) to obtain N-(pyrimidin-2-ylmethyl)-1-[5-(trifluoromethyl)-2-pyridyl]methanamine (2.72 g, 10.13 mmol, 35.49% yield).

LCMS (ESI): [M+H]⁺m/z: calcd 269.1; found 269.0; Rt=0.549 min.

### Step 2: The synthesis of 2,2,2-trifluoroethyl 2-oxo-2-[pyrimidin-2-ylmethyl-[[5-(trifluoromethyl)-2-pyridyl]methyl]amino]acetate

N-(pyrimidin-2-ylmethyl)-1-[5-(trifluoromethyl)-2-pyridyl]methanamine (500 mg, 1.86 mmol) and Triethylamine (216.91 mg, 2.14 mmol, 298.78 μL) were dissolved in DCM (5 mL) and the resulting mixture was cooled to −5° C. in an ice/methanol bath. A solution of 2,2,2-trifluoroethyl 2-chloro-2-oxo-acetate (390.62 mg, 2.05 mmol) in DCM (1.5 mL) was added dropwise to the previous solution and resulting mixture was allowed to warm to room temperature and stirred overnight. Aq. NaHCO₃ solution (15 ml) was added and an organic layer was separated. The aqueous layer was extracted with DCM (20 ml) and combined organic layers were washed with water (15 ml), dried over Na₂SO₄, filtered and concentrated in vacuo to obtain 2,2,2-trifluoroethyl 2-oxo-2-[pyrimidin-2-ylmethyl-[[5-(trifluoromethyl)-2-pyridyl]methyl]amino]acetate (753 mg, 1.78 mmol, 95.66% yield).

LCMS (ESI): [M+H]⁺m/z: calcd 423.1; found 423.0; Rt=1.313 min.

### Step 3: The synthesis of N′-(pyrimidin-2-ylmethyl)-N′-[[5-(trifluoromethyl)-2-pyridyl]methyl]oxamide

2,2,2-trifluoroethyl 2-oxo-2-[pyrimidin-2-ylmethyl-[5-(trifluoromethyl)-2-pyridyl]methyl]amino]acetate (753 mg, 1.78 mmol) was dissolved in NH3/MeOH (35 mL) and the resulting solution was stirred overnight. The reaction mixture was concentrated in vacuo to obtain N′-(pyrimidin-2-ylmethyl)-N′-[5-(trifluoromethyl)-2-pyridyl]methyl]oxamide (643 mg, crude).

LCMS (ESI): [M+H]⁺m/z: calcd 340.1; found 340.1; Rt=0.932 min.

### Step 4: The synthesis of N-[4-amino-1-(2-trimethylsilylethoxymethyl)pyrazolo[4,3-c]pyridin-7-yl]-N′-(pyrimidin-2-ylmethyl)-N′-[5-(trifluoromethyl)-2-pyridyl]methyl]oxamide

N′-(pyrimidin-2-ylmethyl)-N′-[5-(trifluoromethyl)-2-pyridyl]methyl]oxamide (207 mg, 610.13 μmol), 7-bromo-1-(2-trimethylsilylethoxymethyl)pyrazolo[4,3-c]pyridin-4-amine (209.45 mg, 610.13 μmol), Copper (I) iodide (116.20 mg, 610.13 μmol, 20.68 μL), Cesium carbonate (397.58 mg, 1.22 mmol), (1R,2R)—N1,N2-dimethylcyclohexane-1,2-diamine (130.18 mg, 915.19 μmol) and Copper (38.77 mg, 610.13 μmol) were mixed together in DMF (4 mL) and the resulting mixture was purged with argon for 10 min. The resulting mixture was heated at 100° C. overnight. The reaction mixture was filtered and submitted to HPLC and purified (2-10 min 30-60 MeCN+formic acid 30 ml/min) to obtain N-[4-amino-1-(2-trimethylsilylethoxymethyl)pyrazolo[4,3-c]pyridin-7-yl]-N′-(pyrimidin-2-ylmethyl)-N′-[5-(trifluoromethyl)-2-pyridyl]methyl]oxamide (17.6 mg, 29.25 μmol, 4.79% yield) and N-[4-amino-1-(2-trimethylsilylethoxymethyl)pyrazolo[4,3-c]pyridin-7-yl]-N′-(pyrimidin-2-ylmethyl)-N′-[5-(trifluoromethyl)-2-pyridyl]methyl]oxamide (19.8 mg, 32.91 μmol, 5.39% yield).

LCMS (ESI): [M+1]⁺m/z: calcd 602.3; found 602.2; Rt=1.081 min.

### Step 5: The synthesis of N-(4-amino-1H-pyrazolo[4,3-c]pyridin-7-yl)-N′-(pyrimidin-2-ylmethyl)-N′-[5-(trifluoromethyl)-2-pyridyl]methyl]oxamide

N-[4-amino-1-(2-trimethylsilylethoxymethyl)pyrazolo[4,3-c]pyridin-7-yl]-N′-(pyrimidin-2-ylmethyl)-N′-[5-(trifluoromethyl)-2-pyridyl]methyl]oxamide (17.6 mg, 29.25 μmol) was dissolved in TFA (0.5 mL) and the resulting mixture was stirred overnight. The reaction mixture was concentrated in vacuo and purified by HPLC (2-10 min 30-60 MeCN+NH3 30 ml/min) to obtain N-(4-amino-1H-pyrazolo[4,3-c]pyridin-7-yl)-N′-(pyrimidin-2-ylmethyl)-N′-[[5-(trifluoromethyl)-2-pyridyl]methyl]oxamide (4.8 mg, 10.18 μmol, 34.81% yield).

¹H NMR (600 MHz, dmso) 8 4.78-4.97 (m, 2H), 5.20-5.35 (m, 2H), 6.68 (s, 2H), 7.31-7.44 (m, 1H), 7.54-7.64 (m, 1H), 7.64-7.75 (m, 1H), 8.14 (s, 1H), 8.16-8.24 (m, 1H), 8.69-8.82 (m, 2H), 8.84-8.95 (m, 1H), 10.36-10.56 (m, 1H), 12.67-12.85 (m, 1H).

LCMS (ESI): [M+1]⁺m/z: calcd 472.2; found 472.2; Rt=0.921 min.

### Example 12. Synthesis of N-(6-amino-5-methyl-3-pyridyl)-N′-(4-methyltetralin-1-yl)-N′-[[5-(trifluoromethyl)-2-pyridyl]methyl]oxamide (Compound 51)

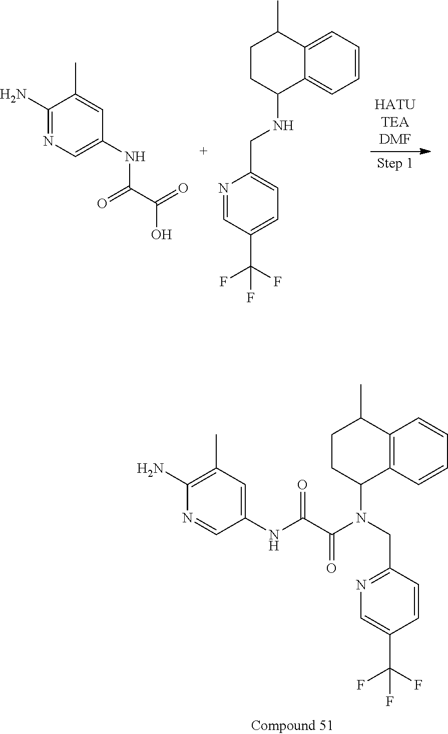

The synthesis of (rac)-4-methyl-N-((5-(trifluoromethyl)pyridin-2-yl)methyl)-1,2,3,4-tetrahydronaphthalen-1-amine is described in Intermediate 4.

### Step 1. The synthesis of N-(6-amino-5-methyl-3-pyridyl)-N′-(4-methyltetralin-1-yl)-N′-[[5-(trifluoromethyl)-2-pyridyl]methyl]oxamide

To the solution of (rac)-4-methyl-N-[5-(trifluoromethyl)-2-pyridyl]methyl]tetralin-1-amine (248.44 mg, 775.51 μmol), 2-[(6-amino-5-methyl-3-pyridyl)amino]-2-oxo-acetic acid (151.36 mg, 775.51 μmol) and Triethylamine (235.42 mg, 2.33 mmol, 324.27 μL) in DMF (3 mL) HATU (324.36 mg, 853.06 μmol) was added portionwise. Mixture was stirred at 25° C. for 2 hr. The reaction mixture was submitted for HPLC (SYSTEM 30-30-80% 0-1-5 min H₂O/ACN/0.1% NH₄OH, flow: 30 ml/min (loading pump 4 ml/min acetonitrile) target mass 497 column: XBridge BEH C18 100×19 mm, 5 μm) to give N-(6-amino-5-methyl-3-pyridyl)-N′-(4-methyltetralin-1-yl)-N′-[5-(trifluoromethyl)-2-pyridyl]methyl]oxamide (52 mg, 104.52 μmol, 13.48% yield) as a white solid.

¹H NMR (DMSO-d6, 600 MHz): δ (ppm) 1.16-1.23 (m, 3H), 1.35-1.78 (m, 3H), 1.74-2.02 (m, 5H), 2.81-2.89 (m, 1H), 3.92-3.95 (m, 1H), 4.66-5.79 (m, 3H), 7.11-7.3 (m, 4H), 7.51-7.62 (m, 2H), 7.87-8.20 (m, 2H), 8.78-8.85 (m, 1H), 10.32-10.70 (m, 1H).

LCMS (ESI): [M+H]⁺m/z: calcd 498.3; found 498.2; Rt=3.257 min.

### Example 13. Synthesis of N-(4-amino-1H-pyrazolo[4,3-c]pyridin-7-yl)-N′-ethyl-N′-[[5-(trifluoromethyl)-2-pyridyl]methyl]oxamide (Compound 11)

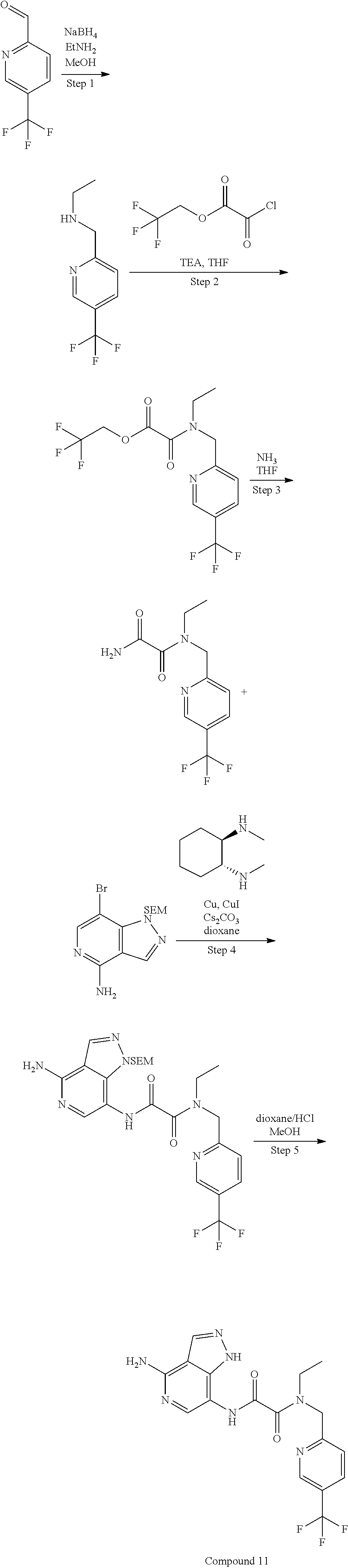

### Step 1: N-[[5-(trifluoromethyl)-2-pyridyl]methyl]ethanamine

ethylamine (1.03 g, 22.84 mmol, 1.28 mL) were added to the solution of 5-(trifluoromethyl)pyridine-2-carbaldehyde (0.2 g, 1.14 mmol) in MeOH (9.36 mL). Resulting mixture was stirred at 60° C. for 1 hour before Sodium Borohydride (86.42 mg, 2.28 mmol, 80.46 μL) was added portions thereto. After that, stirring was continued for 5 hr. Then, solvent was removed under reduced pressure and residue was partitioned between 10% aq. K₂CO₃ solution (20 ml) and DCM (10 ml). Organic layer was separated, dried over solid K₂CO₃ and concentrated under reduced pressure, leaving N-[[5-(trifluoromethyl)-2-pyridyl]methyl]ethanamine (0.12 g, 587.68 μmol, 51.45% yield).

### Step 2:2,2,2-trifluoroethyl 2-[ethyl-[[5-(trifluoromethyl)-2-pyridyl]methyl]amino]-2-oxo-acetate

To a solution of N-[[5-(trifluoromethyl)-2-pyridyl]methyl]ethanamine (0.25 g, 1.22 mmol) and TEA (161.06 mg, 1.59 mmol, 221.84 μL) in THF (9.78 mL) was added 2,2,2-trifluoroethyl 2-chloro-2-oxo-acetate (256.57 mg, 1.35 mmol) dropwise at 0° C. under argon. The reaction mixture was then stirred for 12 hr at r.t., then evaporated in vacuo to give 2,2,2-trifluoroethyl 2-[ethyl-[5-(trifluoromethyl)-2-pyridyl]methyl]amino]-2-oxo-acetate (0.36 g, 1.00 mmol, 82.08% yield).

LCMS (ESI): [M+H]⁺m/z: calcd 359.2; found 359.0; Rt=3.649 min.

### Step 3: N′-ethyl-N′-[[5-(trifluoromethyl)-2-pyridyl]methyl]oxamide

2,2,2-trifluoroethyl 2-[ethyl-[5-(trifluoromethyl)-2-pyridyl]methyl]amino]-2-oxo-acetate (0.36 g, 1.00 mmol) was dissolved in THF (10 mL) and was blow ammonium (17.11 mg, 1.00 mmol). Resulting solution was stirred at 20° C. for 8 hr. The resulting mixture was evaporated in vacuo and residue was triturated with THF (20 ml×2), filtered and combined organic was evaporated in vacuo N′-ethyl-N′-[5-(trifluoromethyl)-2-pyridyl]methyl]oxamide (0.34 g, crude).

LCMS (ESI): [M+H]⁺m/z: calcd 276.2; found 276.2; Rt=1.904 min.

### Step 4: N-[4-amino-1-(2-trimethylsilylethoxymethyl)pyrazolo[4,3-c]pyridin-7-yl]-N′-ethyl-N′-[[5-(trifluoromethyl)-2-pyridyl]methyl]oxamide

A mixture of N′-ethyl-N′-[[5-(trifluoromethyl)-2-pyridyl]methyl]oxamide (200 mg, 726.67 μmol), 7-bromo-1-(2-trimethylsilylethoxymethyl)pyrazolo[4,3-c]pyridin-4-amine (249.46 mg, 726.67 μmol), copper (5 mg, 78.68 μmol), Copper (I) iodide (100 mg, 525.07 μmol, 17.79 μL), cesium carbonate (473.53 mg, 1.45 mmol) and rac-(1R,2R)—N1,N2-dimethylcyclohexane-1,2-diamine (100 mg, 703.04 μmol) in 1,4-dioxane (6 mL) was stirred in a sealed vial under argon at 105° C. for 40 hr. The resulting mixture was cooled down and submitted to reverse phase HPLC (column: XBridge BEH C18 5 μm 130 A; mobile phase: 35-35-85% 0-1-6 min H₂O/MeOH/0.1% NH₄OH, flow rate: 30 ml/min (loading pump 4 ml/min methanol) to afford N-[4-amino-1-(2-trimethylsilylethoxymethyl)pyrazolo[4,3-c]pyridin-7-yl]-N′-ethyl-N′-[5-(trifluoromethyl)-2-pyridyl]methyl]oxamide (77 mg, 143.23 μmol, 19.71% yield) as a light-brown gum.

LCMS (ESI): [M+H]⁺m/z: calcd 538.3; found 538.2; Rt=1.261 min.

### Step 5: The synthesis of N-(4-amino-1H-pyrazolo[4,3-c]pyridin-7-yl)-N′-ethyl-N′-[[5-(trifluoromethyl)-2-pyridyl]methyl]oxamide

Hydrogen chloride solution 4.0M in dioxane (2.10 g, 8.01 mmol, 2 mL, 13.9% purity) was added to a stirred solution of N-[4-amino-1-(2-trimethylsilylethoxymethyl)pyrazolo[4,3-c]pyridin-7-yl]-N′-ethyl-N′-[5-(trifluoromethyl)-2-pyridyl]methyl]oxamide (77 mg, 143.23 μmol) in methanol (2 mL) at 25° C. The resulting solution was stirred at 25° C. for 15 hr, then concentrated to dryness in vacuo and the residue was submitted to reverse phase HPLC (column: Chromatorex 18 SMB100-5T 100×19 mm 5 μm; mobile phase: 30-70% 0-5 min H₂O/MeOH, flow: 30 ml/min (loading pump 4 ml/min methanol)) to afford N-(4-amino-1H-pyrazolo[4,3-c]pyridin-7-yl)-N′-ethyl-N′-[5-(trifluoromethyl)-2-pyridyl]methyl]oxamide (26 mg, 58.58 μmol, 40.90% yield, HCl) as a light-yellow solid.

¹H NMR (500 MHz, dmso) δ 1.06-1.21 (m, 3H), 3.44-3.71 (m, 2H), 4.70-5.11 (m, 2H), 7.59-7.68 (m, 1H), 7.83-8.09 (m, 1H), 8.15-8.27 (m, 1H), 8.30-9.58 (m, 4H), 10.76-11.09 (m, 1H), 12.66-14.31 (m, 1H).

LCMS (ESI): [M+H]⁺m/z: calcd 408.2; found 408.2; Rt=1.954 min.

### Example 14. Synthesis of N1-(4-amino-1H-pyrazolo[4,3-c]pyridin-7-yl)-N2-(4-methyl-1,2,3,4-tetrahydronaphthalen-1-yl)-N2-((5-(trifluoromethyl)pyridin-2-yl)methyl)oxalamide (Compound 3)

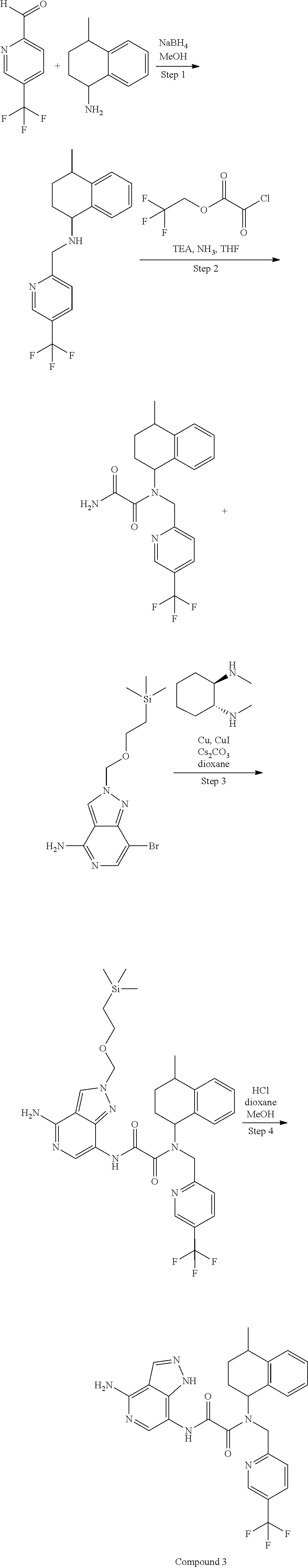

### Step 1: (rac)-4-methyl-N-[[5-(trifluoromethyl)-2-pyridyl]methyl]tetralin-1-amine

5-(trifluoromethyl)pyridine-2-carbaldehyde (2.1 g, 11.99 mmol) 5-(trifluoromethyl)pyridine-2-carbaldehyde (2.1 g, 11.99 mmol) was added to a stirred solution of 4-methyltetralin-1-amine (1.93 g, 11.99 mmol) in Methanol (50 mL) stirred at 20° C. for 2 hr and Sodium Borohydride (226.86 mg, 6.00 mmol, 211.23 μL) added, reaction mixture stirred for 2 hr additional. The reaction mixture was evaporated in vacuo. The residue was dissolved in DCM (50 ml) and washed with water (2*15 ml). Organic layer was dried over Na₂SO₄ and filtered. DCM was evaporated under reduce pressure to give (rac)-4-methyl-N-[[5-(trifluoromethyl)-2-pyridyl]methyl]tetralin-1-amine (2.9 g, 9.05 mmol, 75.48% yield).

LCMS (ESI): [M+H]⁺m/z: calcd 321.2; found 321.2; Rt=0.981 min.

### Step 2: N′-(4-methyltetralin-1-yl)-N′-[5-(trifluoromethyl)-2-pyridyl]methyl]oxamide

2,2,2-trifluoroethyl 2-chloro-2-oxo-acetate (446.01 mg, 2.34 mmol) was slowly added to a stirred solution of (rac)-4-methyl-N-[5-(trifluoromethyl)-2-pyridyl]methyl]tetralin-1-amine (0.5 g, 1.56 mmol) and Triethylamine (315.87 mg, 3.12 mmol, 435.09 μL) in dry THF (10 mL) at 25° C. The resulting mixture was stirred at 25° C. for 0.5 hr, then gaseous ammonia was bubbled through the reaction mixture at 25° C. for 0.5 hr. The resulting ammonium chloride precipitate was filtered and discarded, the filtrate was concentrated in vacuo to afford crude N′-(4-methyltetralin-1-yl)-N′-[5-(trifluoromethyl)-2-pyridyl]methyl]oxamide (0.6 g, 1.53 mmol, 98.22% yield).

LCMS (ESI): [M+H]⁺m/z: calcd 392.2; found 392.0; Rt=1.334 min.

### Step 3: N-[4-amino-2-(2-trimethylsilylethoxymethyl)pyrazolo[4,3-c]pyridin-7-yl]-N′-(4-methyltetralin-1-yl)-N′-[[5-(trifluoromethyl)-2-pyridyl]methyl]oxamide

A mixture of N′-(4-methyltetralin-1-yl)-N′-[5-(trifluoromethyl)-2-pyridyl]methyl]oxamide (0.25 g, 638.76 μmol), 7-bromo-2-(2-trimethylsilylethoxymethyl)pyrazolo[4,3-c]pyridin-4-amine (219.28 mg, 638.76 μmol), Copper (I) iodide (121.65 mg, 638.76 μmol, 21.65 μL), Cesium carbonate (416.24 mg, 1.28 mmol),

(1R,2R)—N1,N2-dimethylcyclohexane-1,2-diamine (136.29 mg, 958.13 μmol) and Cu(4.06 mg, 63.88 μmol) in Dioxane (5 mL) was stirred under argon at 90° C. for 14 hr. The reaction mixture was submitted for reverse phase HPLC (SYSTEM 60-100% 0-1-6 min H₂O/ACN/0.1% NH₄OH, flow: 30 ml/min (loading pump 4 ml/min methanol) target mass 564 column: XBridge BEH C18 100×19 mm, 5 μm) to give N-[4-amino-2-(2-trimethylsilylethoxymethyl)pyrazolo[4,3-c]pyridin-7-yl]-N′-(4-methyltetralin-1-yl)-N′-[5-(trifluoromethyl)-2-pyridyl]methyl]oxamide (34 mg, 52.01 μmol, 8.14% yield).

LCMS (ESI): [M+H]⁺m/z: calcd 654.3; found 654.0; Rt=1.448 min.

### Step 4: The synthesis of N1-(4-amino-1H-pyrazolo[4,3-c]pyridin-7-yl)-N2-(4-methyl-1,2,3,4-tetrahydronaphthalen-1-yl)-N2-((5-(trifluoromethyl)pyridin-2-yl)methyl)oxalamide

To the stirred solution of N-[4-amino-2-(2-trimethylsilylethoxymethyl)pyrazolo[4,3-c]pyridin-7-yl]-N′-(4-methyltetralin-1-yl)-N′-[5-(trifluoromethyl)-2-pyridyl]methyl]oxamide (34 mg, 52.01 μmol) in MeOH (0.5 mL) Hydrogen chloride solution 4.0M in dioxane (400.00 mg, 10.97 mmol, 0.5 mL) was added and the resulting mixture was stirred at 25° C. for 15 hr. The reaction mixture was concentrated under reduce pressure. The residue was purified by reverse phase HPLC (SYSTEM 40-90% 0-6 min H₂O/MeOH/0.1% NH₄OH, flow: 30 ml/min (loading pump 4 ml/min methanol) target mass 523.52 column: XBridge BEH C18 100×19 mm, 5 μm) to give N1-(4-amino-1H-pyrazolo[4,3-c]pyridin-7-yl)-N2-(4-methyl-1,2,3,4-tetrahydronaphthalen-1-yl)-N2-((5-(trifluoromethyl)pyridin-2-yl)methyl)oxalamide (14 mg, 26.74 μmol, 51.42% yield) as a white solid.

¹H NMR (600 MHz, dmso) δ 1.20 (dd, 3H), 1.34-1.62 (m, 1H), 1.61-1.84 (m, 1H), 1.84-2.26 (m, 2H), 2.80-2.94 (m, 1H), 3.38-4.02 (m, 1H), 4.42-4.82 (m, 1H), 5.00-5.68 (m, 1H), 5.69-6.76 (m, 2H), 7.05-7.15 (m, 1H), 7.15-7.25 (m, 2H), 7.25-7.38 (m, 1H), 7.43-7.79 (m, 2H), 7.78-8.25 (m, 2H), 8.62-8.90 (m, 1H), 9.75-10.83 (m, 1H), 12.58-13.57 (m, 1H).

LCMS (ESI): [M+H]⁺m/z: calcd 524.2; found 524.2; Rt=3.056 min.

### Example 15. Synthesis of (R)—N¹-(5-carbamoyl-6-methoxypyridin-3-yl)-N2-(1-cyclopropyl-2-methoxyethyl)-N2-((5-(trifluoromethyl)pyridin-2-yl)methyl)oxalamide (Compound 37)

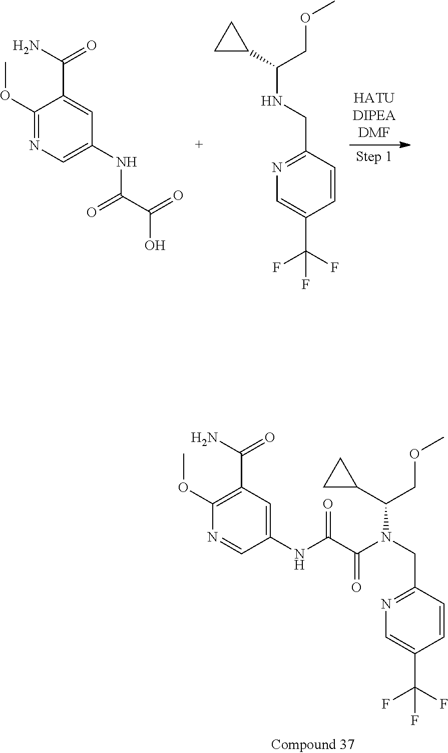 [SMILES: COc1ncc(NC(=O)C(=O)O)cc1C(N)=O ; COC[C@H](NCc1ccc(C(F)(F)F)cn1)C1CC1 ; COC[C@@H](C1CC1)N(Cc1ccc(C(F)(F)F)cn1)C(=O)C(=O)Nc1cnc(OC)c(C(N)=O)c1]

The synthesis of (R)-1-cyclopropyl-2-methoxy-N-((5-(trifluoromethyl)pyridin-2-yl)methyl) ethan-1-amine is described in Intermediate 1.

### Step 1. The synthesis of (R)—N¹-(5-carbamoyl-6-methoxypyridin-3-yl)-N2-(1-cyclopropyl-2-methoxyethyl)-N2-((5-(trifluoromethyl)pyridin-2-yl)methyl)oxalamide

To a stirred solution of (1R)-1-cyclopropyl-2-methoxy-N-[[5-(trifluoromethyl)-2-pyridyl]methyl]ethanamine (0.1 g, 364.59 μmol), 2-[(5-carbamoyl-6-methoxy-3-pyridyl)amino]-2-oxo-acetic acid (0.1 g, 418.09 μmol) and N,N-Diisopropylethylamine (148.40 mg, 1.15 mmol, 0.2 mL) in DMF (5 mL) was added HATU (0.15 g, 394.50 μmol) at 5-10° C. The resulting reaction mixture was stirred at 25° C. for 16 hr. Upon completion, the reaction mixture was concentrated under reduced pressure to obtain crude product. The obtained crude product was purified by reverse phase HPLC chromatography (Device (Mobile Phase, Column): SYSTEM 20-20-45% 0-1-5 min H₂O/ACN, flow: 30 ml/min (loading pump 4 ml/min acetonitrile) target mass 495 column: Chromatorex 18 SMB 100-5T 100×19 mm 5 μm) to afford (R)—N¹-(5-carbamoyl-6-methoxypyridin-3-yl)-N2-(1-cyclopropyl-2-methoxyethyl)-N2-((5-(trifluoromethyl)pyridin-2-yl)methyl)oxalamide (94 mg, 189.73 μmol, 52.04% yield).

¹H NMR (DMSO-d6, 600 MHz): δ (ppm) 0.11-0.54 (m, 4H), 0.95-0.96 (m, 1H), 3.09-3.14 (m, 3H), 3.43-3.59 (m, 3H), 3.79-3.94 (m, 3H), 4.77-5.11 (m, 2H), 7.58-7.77 (m, 3H), 8.15-8.22 (m, 1H), 8.38-8.53 (m, 2H), 8.80-8.90 (m, 1H), 10.83-10.91 (m, 1H).

LCMS (ESI): [M+H]⁺m/z: calcd 496.2; found 496.2; Rt=2.081 min.

### Example 16. Synthesis of (R)—N¹-(5-carbamoyl-6-methoxypyridin-3-yl)-N2-(2,3-dihydro-1H-inden-1-yl)-N2-((5-(trifluoromethyl)pyridin-2-yl)methyl)oxalamide (Compound 30)

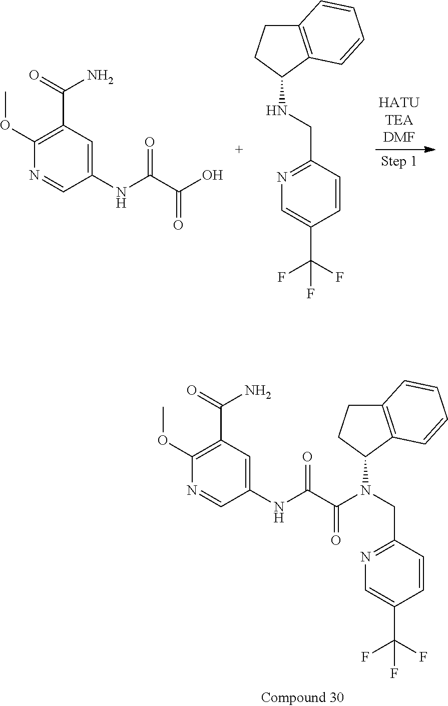 [SMILES: COc1ncc(NC(=O)C(=O)O)cc1C(N)=O ; FC(F)(F)c1ccc(CN[C@@H]2CCc3ccccc32)nc1 ; COc1ncc(NC(=O)C(=O)N(Cc2ccc(C(F)(F)F)cn2)[C@@H]2CCc3ccccc32)cc1C(N)=O]

The synthesis of (R)—N-((5-(trifluoromethyl)pyridin-2-yl)methyl)-2,3-dihydro-1H-inden-1-amine is above is described in Intermediate 2.

### Step 1. The synthesis of (R)—N¹-(5-carbamoyl-6-methoxypyridin-3-yl)-N2-(2,3-dihydro-1H-inden-1-yl)-N2-((5-(trifluoromethyl)pyridin-2-yl)methyl)oxalamide

HATU (214.64 mg, 564.49 μmol) was added portionwise at r.t. to a suspension of 2-[(5-carbamoyl-6-methoxy-3-pyridyl)amino]-2-oxo-acetic acid (135.02 mg, 564.49 μmol), (1R)—N-[[5-(trifluoromethyl)-2-pyridyl]methyl]indan-1-amine (150 mg, 513.17 μmol) and TEA (311.57 mg, 3.08 mmol, 429.16 μL) in DMF (6 mL). The clear solution was stirred at 20° C. for 18 hr and the solvents were evaporated in vacuo. The residue was purified by RP HPLC (column: XBridge C18 100×19 mm, 5 μm; mobile phase: 30-65% 0-5 min H₂O/ACN/0.1% NH₄OH, flow: 30 ml/min (loading pump 4 ml/min methanol)) to give 63 mg of crude product 91% purity by

LCMS, which was repurified by reverse phase HPLC (column: XBridge BEH C18 100×19 mm, 5 μm; mobile phase: 35-85% 0-6 min H₂O/MeOH/0.1% NH₄OH, flow: 30 ml/min (loading pump 4 ml/min acetonitrile) to afford (R)—N¹-(5-carbamoyl-6-methoxypyridin-3-yl)-N2-(2,3-dihydro-1H-inden-1-yl)-N2-((5-(trifluoromethyl)pyridin-2-yl)methyl)oxalamide (48 mg, 93.48 μmol, 18.22% yield) as a pink solid.

¹H NMR (DMSO-d6, 600 MHz): δ (ppm) 1.96 (m, 1H), 2.39 (m, 1H), 2.84 (m, 2H), 3.93 (m, 3H), 4.59 (m, 2H), 5.89 (m, 1H), 7.15 (m, 2H), 7.22 (m, 2H), 7.50 (m, 1H), 7.70 (dd, 2H), 8.12 (m, 1H), 8.48 (m, 2H), 8.80 (m, 1H), 11.05 (m, 1H).

LCMS (ESI): [M+H]⁺m/z: calcd 514.2; found 514.0; Rt=3.267 min.

### Example 17. Synthesis of (R)—N¹-(6-amino-5-methylpyridin-3-yl)-N2-(2,3-dihydro-1H-inden-1-yl)-N2-((5-(trifluoromethyl)pyridin-2-yl)methyl)oxalamide (Compound 1)

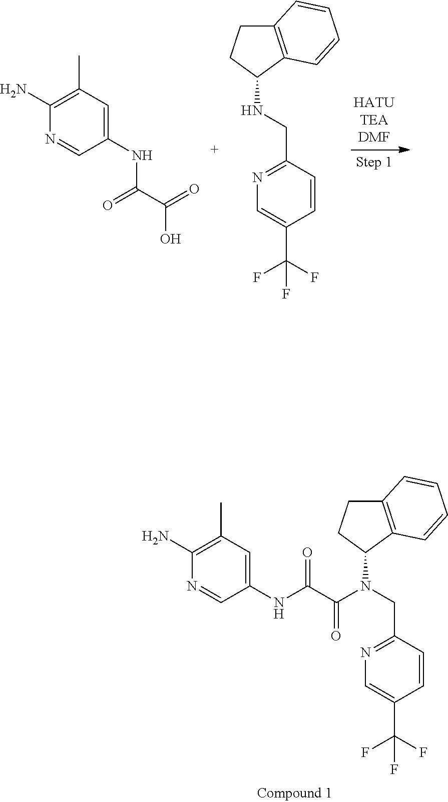 [SMILES: Cc1cc(NC(=O)C(=O)O)cnc1N ; FC(F)(F)c1ccc(CN[C@@H]2CCc3ccccc32)nc1 ; Cc1cc(NC(=O)C(=O)N(Cc2ccc(C(F)(F)F)cn2)[C@@H]2CCc3ccccc32)cnc1N]

The synthesis of (1R)—N-((5-(trifluoromethyl)pyridin-2-yl)methyl)-2,3-dihydro-1H-inden-1-amine is above is described in Intermediate 2.

### Step 1. The synthesis of (R)—N¹-(6-amino-5-methylpyridin-3-yl)-N2-(2,3-dihydro-1H-inden-1-yl)-N2-((5-(trifluoromethyl)pyridin-2-yl)methyl)oxalamide

HATU (214.64 mg, 564.49 μmol) was added portionwise at r.t. to a suspension of 2-[(6-amino-5-methyl-3-pyridyl)amino]-2-oxo-acetic acid (110.17 mg, 564.49 μmol), (1R)—N-[5-(trifluoromethyl)-2-pyridyl]methyl]indan-1-amine (150 mg, 513.17 μmol) and TEA (311.57 mg, 3.08 mmol, 429.16 μL) in DMF (6 mL). The clear solution was stirred at 20° C. for 18 hr and the solvents were evaporated in vacuo. The residue was purified by RP HPLC ((column: XBridge C18 100×19 mm, 5 μm; mobile phase: 30-55% 0-5 min H₂O/Acetonitrile/0.1% NH₄OH; flow: 30 ml/min (loading pump 4 ml/min acetonitrile)) to give 65 mg of crude product 85% purity by LCMS, which was repurified by reverse phase HPLC (column: XBridge C18 100×19 mm, 5 μm; mobile phase: 50-75% 0-5 min H₂O/MeOH/0.1% NH₄OH, flow: 30 ml/min (loading pump 4 ml/min methanol) to afford (R)—N¹-(6-amino-5-methylpyridin-3-yl)-N2-(2,3-dihydro-1H-inden-1-yl)-N2-((5-(trifluoromethyl)pyridin-2-yl)methyl)oxalamide (47 mg, 100.12 μmol, 19.51% yield) as a beige solid.

¹H NMR (DMSO-d6, 600 MHz): δ (ppm) 1.99 (m, 4H), 2.30 (m, 1H), 2.78 (m, 1H), 2.86 (m, 1H), 4.55 (m, 2H), 5.60 (m, 2H), 5.86 (m, 1H), 7.11 (m, 1H), 7.18 (m, 1H), 7.31 (m, 2H), 7.51 (m, 2H), 8.05 (m, 2H), 8.79 (m, 1H), 10.51 (m, 1H).

LCMS (ESI): [M+H]⁺m/z: calcd 470.2; found 470.2; Rt=3.003 min.

### Example 18. Synthesis of (R)—N¹-(6-amino-5-methylpyridin-3-yl)-N2-(1-cyclopropyl-2-methoxyethyl)-N2-((5-(trifluoromethyl)pyridin-2-yl)methyl)oxalamide (Compound 5)

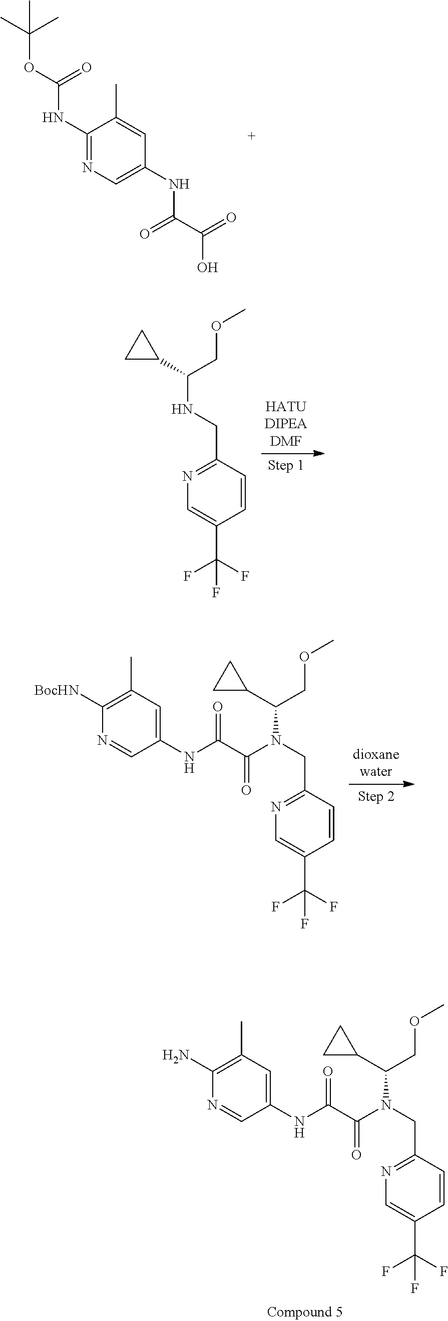 [SMILES: Cc1cc(NC(=O)C(=O)O)cnc1NC(=O)OC(C)(C)C ; COC[C@H](NCc1ccc(C(F)(F)F)cn1)C1CC1 ; COC[C@@H](C1CC1)N(Cc1ccc(C(F)(F)F)cn1)C(=O)C(=O)Nc1cnc(NC(=O)OC(C)(C)C)c(C)c1 ; COC[C@@H](C1CC1)N(Cc1ccc(C(F)(F)F)cn1)C(=O)C(=O)Nc1cnc(N)c(C)c1]

The synthesis of (R)-1-cyclopropyl-2-methoxy-N-((5-(trifluoromethyl)pyridin-2-yl)methyl) ethan-1-amine is described in Intermediate 1.

### Step 1: (R)-tert-butyl(5-(2-((1-cyclopropyl-2-methoxyethyl)((5-(trifluoromethyl)pyridin-2-yl)methyl)amino)-2-oxoacetamido)-3-methylpyridin-2-yl)carbamate

To a stirred solution of (1R)-1-cyclopropyl-2-methoxy-N-[5-(trifluoromethyl)-2-pyridyl]methyl]ethanamine (0.1 g, 364.59 μmol), 2-[6-(tert-butoxycarbonylamino)-5-methyl-3-pyridyl]amino]-2-oxo-acetic acid (0.12 g, 406.38 μmol) and N,N-Diisopropylethylamine (148.40 mg, 1.15 mmol, 0.2 mL) in DMF (5 mL) was added HATU (0.15 g, 394.50 μmol) at 5-10° C. The resulting reaction mixture was stirred at 25° C. for 16 hr. Upon completion, the reaction mixture was concentrated under reduced pressure to obtain crude product. The desired product (R)-tert-butyl(5-(2-((1-cyclopropyl-2-methoxyethyl)((5-(trifluoromethyl)pyridin-2-yl)methyl)amino)-2-oxoacetamido)-3-methylpyridin-2-yl)carbamate (0.2 g, 362.61 μmol, 99.46% yield) was isolated.

LCMS (ESI): [M+H]⁺m/z: calcd 552.3; found 552.2; Rt=1.459 min.

### Step 2: The synthesis of ((R)—N¹-(6-amino-5-methylpyridin-3-yl)-N2-(1-cyclopropyl-2-methoxyethyl)-N2-((5-(trifluoromethyl)pyridin-2-yl)methyl)oxalamide

To a stirred solution of (1R)-1-cyclopropyl-2-methoxy-N-[5-(trifluoromethyl)-2-pyridyl]methyl]ethanamine (0.1 g, 364.59 μmol), 2-[[6-(tert-butoxycarbonylamino)-5-methyl-3-pyridyl]amino]-2-oxo-acetic acid (0.12 g, 406.38 μmol) and N,N-Diisopropylethylamine (148.40 mg, 1.15 mmol, 0.2 mL) in DMF (5 mL) was added HATU (0.15 g, 394.50 μmol) at 5-10° C. The resulting reaction mixture was stirred at 25° C. for 16 hr. Upon completion, the reaction mixture was concentrated under reduced pressure to obtain crude product. The desired product (R)—N¹-(6-amino-5-methylpyridin-3-yl)-N2-(1-cyclopropyl-2-methoxyethyl)-N2-((5-(trifluoromethyl)pyridin-2-yl)methyl)oxalamide (0.2 g, 362.61 μmol, 99.46% yield) was isolated.

¹H NMR (DMSO-d6, 600 MHz): δ (ppm) 0.12-0.54 (m, 4H), 0.94-0.96 (m, 1H), 1.96-2.02 (m, 3H), 3.09-3.13 (m, 3H), 3.39-3.80 (m, 2H), 4.74-4.86 (m, 2H), 5.00-5.07 (m, 1H), 5.56-5.61 (m, 2H), 7.28-8.01 (m, 3H), 8.12-8.20 (m, 1H), 8.82-8.88 (m, 1H), 10.28-10.38 (m, 1H).

LCMS (ESI): [M+H]⁺m/z: calcd 552.3; found 552.2; Rt=1.459 min.

### Example 19. Synthesis of (R)—N¹-(5-carbamoylpyridin-3-yl)-N2-(1-cyclopropyl-2-methoxyethyl)-N2-((5-(trifluoromethyl)pyridin-2-yl)methyl)oxalamide (Compound 34)

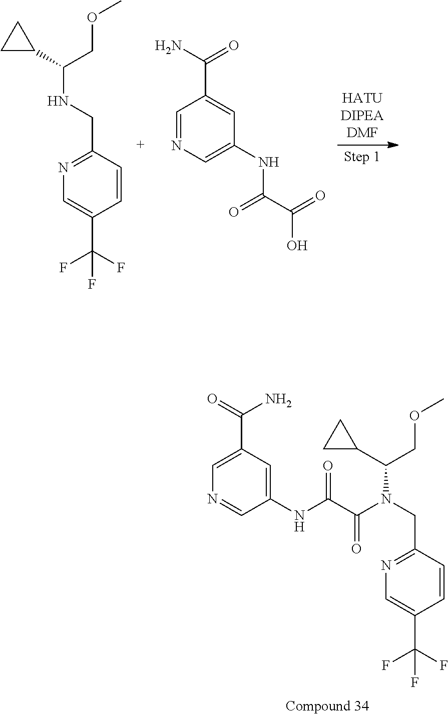 [SMILES: COC[C@H](NCc1ccc(C(F)(F)F)cn1)C1CC1 ; NC(=O)c1cncc(NC(=O)C(=O)O)c1 ; COC[C@@H](C1CC1)N(Cc1ccc(C(F)(F)F)cn1)C(=O)C(=O)Nc1cncc(C(N)=O)c1]

The synthesis of (R)-1-cyclopropyl-2-methoxy-N-((5-(trifluoromethyl)pyridin-2-yl)methyl)ethanamine is described in Intermediate 1.

### Step 1: The synthesis of (R)—N¹-(5-carbamoylpyridin-3-yl)-N2-(1-cyclopropyl-2-methoxyethyl)-N2-((5-(trifluoromethyl)pyridin-2-yl)methyl)oxalamide

To a stirred solution of (1R)-1-cyclopropyl-2-methoxy-N-[[5-(trifluoromethyl)-2-pyridyl]methyl]ethanamine (0.1 g, 364.59 μmol), 2-[(5-carbamoyl-3-pyridyl)amino]-2-oxo-acetic acid (0.1 g, 407.13 μmol, HCl) and N,N-Diisopropylethylamine (188.48 mg, 1.46 mmol, 254.02 μL) in DMF (5 mL) was added HATU (0.15 g, 394.50 μmol) at 5-10° C. The resulting reaction mixture was stirred at 25° C. for 16 hr. Upon completion, the reaction mixture was concentrated under reduced pressure to obtain crude product. The obtained crude product was purified by reverse phase HPLC chromatography (Device (Mobile Phase, Column): SYSTEM 20-20-50% 0-1-5 min H₂O/ACN, flow: 30 ml/min (loading pump 4 ml/min acetonitrile) target mass 465 column: Chromatorex 18 SMB100-5T 100×19 mm 5 μm) to afford (R)—N¹-(5-carbamoylpyridin-3-yl)-N2-(1-cyclopropyl-2-methoxyethyl)-N2-((5-(trifluoromethyl)pyridin-2-yl)methyl)oxalamide (70 mg, 150.40 μmol, 41.25% yield) as a white solid.

¹H NMR (DMSO-d6, 600 MHz): δ (ppm) 0.11-0.55 (m, 4H), 0.94-0.98 (m, 1H), 3.09-3.14 (m, 3H), 3.43-3.83 (m, 3H), 4.78-5.12 (m, 2H), 7.56-7.78 (m, 2H), 8.09-8.23 (m, 2H), 8.40-8.50 (m, 1H), 8.72-8.90 (m, 3H), 11.02-11.11 (m, 1H).

LCMS (ESI): [M+H]⁺m/z: calcd 466.2; found 466.2; Rt=2.611 min.

### Example 20. Synthesis of (R)—N¹-(6-amino-5-ethylpyridin-3-yl)-N2-(2,3-dihydro-1H-inden-1-yl)-N2-((5-(trifluoromethyl)pyridin-2-yl)methyl)oxalamide (Compound 36)

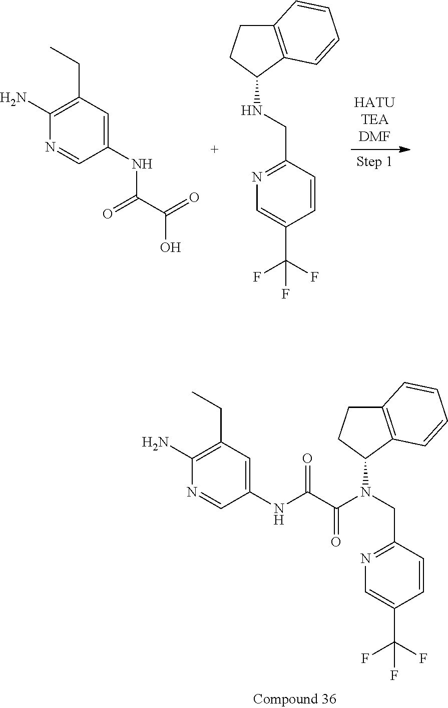 [SMILES: CCc1cc(NC(=O)C(=O)O)cnc1N ; FC(F)(F)c1ccc(CN[C@@H]2CCc3ccccc32)nc1 ; CCc1cc(NC(=O)C(=O)N(Cc2ccc(C(F)(F)F)cn2)[C@@H]2CCc3ccccc32)cnc1N]

The synthesis of (R)—N-((5-(trifluoromethyl)pyridin-2-yl)methyl)-2,3-dihydro-1H-inden-1-amine is above is described in Intermediate 2.

### Step 1. Synthesis of (R)—N¹-(6-amino-5-ethylpyridin-3-yl)-N2-(2,3-dihydro-1H-inden-1-yl)-N2-((5-(trifluoromethyl)pyridin-2-yl)methyl)oxalamide

HATU (214.64 mg, 564.49 μmol) was added portionwise at r.t. to a suspension of 2-[(6-amino-5-ethyl-3-pyridyl)amino]-2-oxo-acetic acid (118.09 mg, 564.49 μmol), (1R)—N-[[5-(trifluoromethyl)-2-pyridyl]methyl]indan-1-amine (150 mg, 513.17 μmol) and TEA (311.57 mg, 3.08 mmol, 429.16 μL) in DMF (6 mL). The clear solution was stirred at 20° C. for 18 hr and the solvents were evaporated in vacuo. The residue was purified by RP HPLC (column: XBridge C18 100×19 mm, 5 μm; mobile phase: 40-65% 0-5 min H₂O/ACN/0.1% NH₄OH; flow: 30 ml/min (loading pump 4 ml/min methanol)) to give 85 mg of crude product 92% purity by LCMS, which was repurified by reverse phase HPLC (column: Chromatorex 18 SMB 100-5T 100×19 mm 5 μm; mobile phase: 40-90% 0-6 min H₂O/MeOH/0.1% NH₄OH, flow: 30 ml/min (loading pump 4 ml/min acetonitrile) to afford (R)—N¹-(6-amino-5-ethylpyridin-3-yl)-N2-(2,3-dihydro-1H-inden-1-yl)-N2-((5-(trifluoromethyl)pyridin-2-yl)methyl)oxalamide (68 mg, 140.65 μmol, 27.41% yield) as a light-brown solid.

¹H NMR (DMSO-d6, 600 MHz): δ (ppm) 1.08 (m, 3H), 1.94 (m, 1H), 2.33 (m, 1H), 2.39 (m, 2H), 2.83 (m, 2H), 4.56 (m, 2H), 5.62 (m, 2H), 5.87 (m, 1H), 7.20 (m, 4H), 7.47 (m, 2H), 8.07 (m, 2H), 8.79 (m, 1H), 10.51 (m, 1H).

LCMS (ESI): [M+H]⁺m/z: calcd 484.2; found 484.2; Rt=3.109 min.

### Example 21. Synthesis of N1-(5-carbamoylpyridin-3-yl)-N2-((1R,2R)-2-(difluoromethoxy)cyclopentyl)-N2-((5-(trifluoromethyl)pyridin-2-yl)methyl)oxalamide (Compound 22)

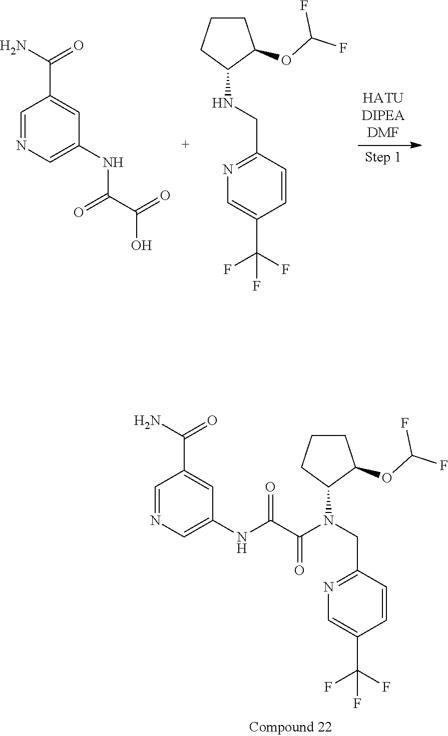 [SMILES: NC(=O)c1cncc(NC(=O)C(=O)O)c1 ; FC(F)O[C@@H]1CCC[C@H]1NCc1ccc(C(F)(F)F)cn1 ; NC(=O)c1cncc(NC(=O)C(=O)N(Cc2ccc(C(F)(F)F)cn2)[C@@H]2CCC[C@H]2OC(F)F)c1]

The synthesis of (1R,2R)-2-(difluoromethoxy)-N-((5-(trifluoromethyl)pyridin-2-yl)methyl)cyclopentan-1-amine is described in Intermediate 3.

### Step 1. The synthesis of N1-(5-carbamoylpyridin-3-yl)-N2-((1R,2R)-2-(difluoromethoxy)cyclopentyl)-N2-((5-(trifluoromethyl)pyridin-2-yl)methyl)oxalamide

DIPEA (368.33 mg, 2.85 mmol, 496.41 μL) was added to the solution of respective 2-[(5-carbamoyl-3-pyridyl)amino]-2-oxo-acetic acid (0.2 g, 814.27 μmol, HCl) and (1R,2R)-2-(difluoromethoxy)-N-[5-(trifluoromethyl)-2-pyridyl]methyl]cyclopentanamine (252.64 mg, 814.27 μmol) in DMF (4.50 mL). The resulting mixture was stirred for 5 min followed by the addition of HATU (340.57 mg, 895.69 μmol). Then, the reaction mixture was stirred overnight at rt. After the completion of the reaction, monitored by LCMS, the resulting suspension was concentrated under reduced pressure. It was dissolved in EtOAc (20 ml) and washed with water (5 ml), brine (5 ml), dried over Na₂SO₄ and evaporated. The obtained solid was subjected to HPLC (Waters Sunfire C18 20*100 5 mkm column and MeOH+NH3 as an eluent mixture) to afford N1-(5-carbamoylpyridin-3-yl)-N2-((1R,2R)-2-(difluoromethoxy)cyclopentyl)-N2-((5-(trifluoromethyl)pyridin-2-yl)methyl)oxalamide (29 mg, 57.84 μmol, 7.10% yield) as a white solid.

¹H NMR (DMSO-d6, 600 MHz): δ (ppm) 1.63-2.06 (m, 7H), 4.31-4.81 (m, 3H), 6.43-6.75 (m, 1H), 7.52-7.68 (m, 2H), 8.09-8.21 (m, 2H), 8.39-8.52 (m, 1H), 8.70-8.92 (m, 3H), 11.13 (br s, 1H).

LCMS (ESI): [M+H]⁺m/z: calcd 502.2; found 502.2; Rt=3.123 min.

### Example 22. Synthesis of (R)—N¹-(6-amino-5-cyclopropylpyridin-3-yl)-N2-(2,3-dihydro-1H-inden-1-yl)-N2-((5-(trifluoromethyl)pyridin-2-yl)methyl)oxalamide (Compound 23)

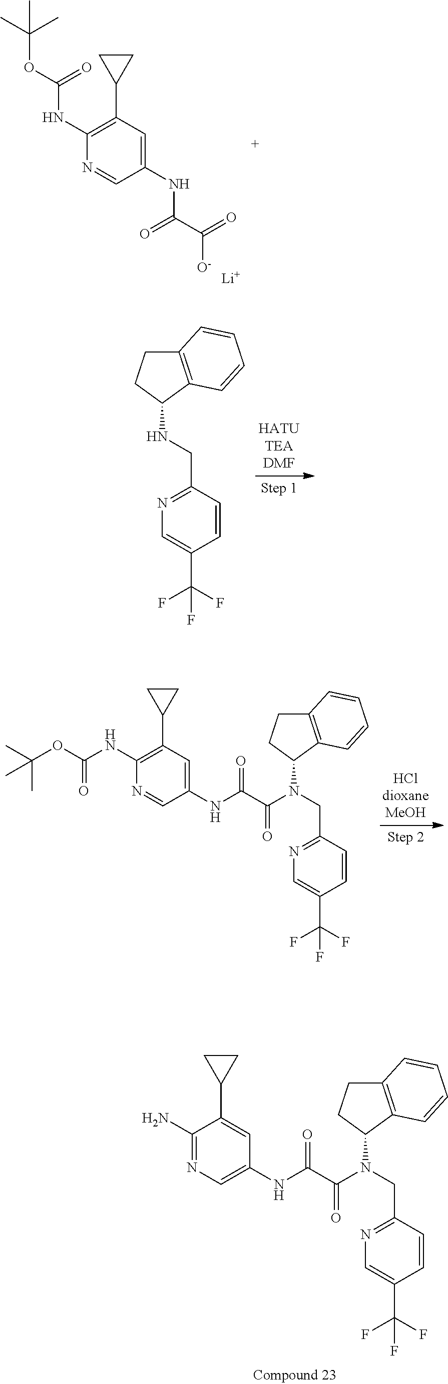 [SMILES: CC(C)(C)OC(=O)Nc1ncc(NC(=O)C(=O)[O-])cc1C1CC1 ; [Li+] ; FC(F)(F)c1ccc(CN[C@@H]2CCc3ccccc32)nc1 ; CC(C)(C)OC(=O)Nc1ncc(NC(=O)C(=O)N(Cc2ccc(C(F)(F)F)cn2)[C@@H]2CCc3ccccc32)cc1C1CC1 ; Nc1ncc(NC(=O)C(=O)N(Cc2ccc(C(F)(F)F)cn2)[C@@H]2CCc3ccccc32)cc1C1CC1]

The synthesis of (1R)—N-[[5-(trifluoromethyl)-2-pyridyl]methyl]indan-1-amine is described in Intermediate 2.

### Step 1: (R)-tert-butyl(3-cyclopropyl-5-(2-((2,3-dihydro-1H-inden-1-yl)((5-(trifluoromethyl)pyridin-2-yl)methyl)amino)-2-oxoacetamido)pyridin-2-yl)carbamate

HATU (214.64 mg, 564.49 μmol) was added portionwise at r.t. to a suspension of lithium;2-[[6-(tert-butoxycarbonylamino)-5-cyclopropyl-3-pyridyl]amino]-2-oxo-acetate (218.32 mg, 667.13 μmol), (1R)—N-[[5-(trifluoromethyl)-2-pyridyl]methyl]indan-1-amine (150 mg, 513.17 μmol) and TEA (311.57 mg, 3.08 mmol, 429.16 μL) in DMF (6 mL). The clear solution was stirred at 20° C. for 18 hr and the solvents were evaporated in vacuo. The residue was dissolved in EtOAc (30 mL), washed with water (5×10 mL) and evaporated in vacuo to give (R)-tert-butyl(3-cyclopropyl-5-(2-((2,3-dihydro-1H-inden-1-yl)((5-(trifluoromethyl)pyridin-2-yl)methyl)amino)-2-oxoacetamido)pyridin-2-yl)carbamate (0.3 g, crude) as a brown solid.

LCMS (ESI): [M+H]⁺m/z: calcd 596.3; found 596.2; Rt=1.484 min.

### Step 2: The synthesis of (R)—N¹-(6-amino-5-cyclopropylpyridin-3-yl)-N2-(2,3-dihydro-1H-inden-1-yl)-N2-((5-(trifluoromethyl)pyridin-2-yl)methyl)oxalamide

To a solution of tert-butyl N-[3-cyclopropyl-5-[[2-oxo-2-[(1R)-indan-1-yl]-[5-(trifluoromethyl)-2-pyridyl]methyl]amino]acetyl]amino]-2-pyridyl]carbamate (0.3 g, 141.03 μmol) in DCM (1.96 mL) was added Hydrogen chloride solution 4.0M in dioxane (734.59 mg, 2.82 mmol, 693.01 μL, 14% purity) at 20° C. The resulting mixture was left to stir for 18 hr. The resulting mixture was evaporated to dryness. The residue was purified by RP-HPLC (column: XBridge BEH C18 100×19 mm, 5 μm; 35-60% 0-5 min H₂O/ACN/0.1% NH₄OH, flow: 30 ml/min) to give (R)—N¹-(6-amino-5-cyclopropylpyridin-3-yl)-N2-(2,3-dihydro-1H-inden-1-yl)-N2-((5-(trifluoromethyl)pyridin-2-yl)methyl)oxalamide (11.6 mg, 23.41 μmol, 16.60% yield) as a yellow solid.

¹H NMR (600 MHz, dmso) 8 0.36-0.48 (m, 2H), 0.83-0.89 (m, 2H), 1.59-1.68 (m, 1H), 1.86-2.01 (m, 1H), 2.24-2.36 (m, 1H), 2.38-2.42 (m, 1H), 2.74-2.81 (m, 1H), 2.84-2.92 (m, 1H), 4.07-4.61 (m, 1H), 4.61-5.70 (m, 1H), 5.70-5.80 (m, 2H), 7.07-7.14 (m, 1H), 7.14-7.19 (m, 1H), 7.19-7.25 (m, 2H), 7.33-7.39 (m, 1H), 7.47-7.53 (m, 1H), 7.89-8.19 (m, 2H), 8.75-8.82 (m, 1H), 10.29-10.67 (m, 1H).

LCMS (ESI): [M+H]⁺m/z: calcd 496.2; found 496.2; Rt=2.598 min.

### Example 23. Synthesis of N-(6-amino-5-ethyl-3-pyridyl)-N′-(4-methyltetralin-1-yl)-N′-[[5-(trifluoromethyl)-2-pyridyl]methyl]oxamide (Compound 10)

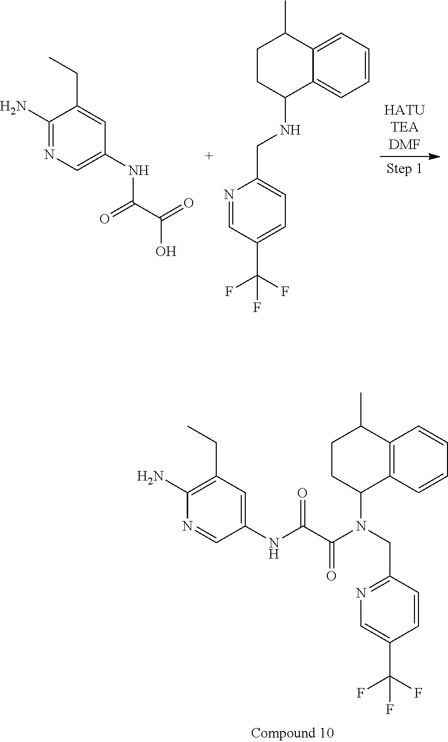

The synthesis of (rac)-4-methyl-N-((5-(trifluoromethyl)pyridin-2-yl)methyl)-1,2,3,4-tetrahydronaphthalen-1-amine is described in Intermediate 4.

### Step 1. The synthesis of N-(6-amino-5-ethyl-3-pyridyl)-N′-(4-methyltetralin-1-yl)-N′-[[5-(trifluoromethyl)-2-pyridyl]methyl]oxamide

To the solution of (rac)-4-methyl-N-[[5-(trifluoromethyl)-2-pyridyl]methyl]tetralin-1-amine (248.44 mg, 775.51 μmol), 2-[(6-amino-5-ethyl-3-pyridyl)amino]-2-oxo-acetic acid (162.24 mg, 775.51 μmol) and Triethylamine (235.42 mg, 2.33 mmol, 324.27 μL) in DMF (3 mL) HATU (324.36 mg, 853.06 μmol) was added portionwise. Mixture was stirred at 25° C. for 2 hr. The reaction mixture was submitted for HPLC (SYSTEM 30-30-80% 0-1-5 min H₂O/ACN/0.1% NH₄OH, flow: 30 ml/min (loading pump 4 ml/min acetonitrile) target mass 511 column: XBridge BEH C18 100×19 mm, 5 μm) to give N-(6-amino-5-ethyl-3-pyridyl)-N′-(4-methyltetralin-1-yl)-N′-[5-(trifluoromethyl)-2-pyridyl]methyl]oxamide (49 mg, 95.79 μmol, 12.35% yield) as a white solid.

LCMS (ESI): [M+H]⁺m/z: calcd 512.3; found 512.2; Rt=3.360 min.

### Example 24. Synthesis of N1-(6-amino-5-cyclopropylpyridin-3-yl)-N2-((1R,2R)-2-(difluoromethoxy)cyclopentyl)-N2-((5-(trifluoromethyl)pyridin-2-yl)methyl)oxalamide (Compound 4)

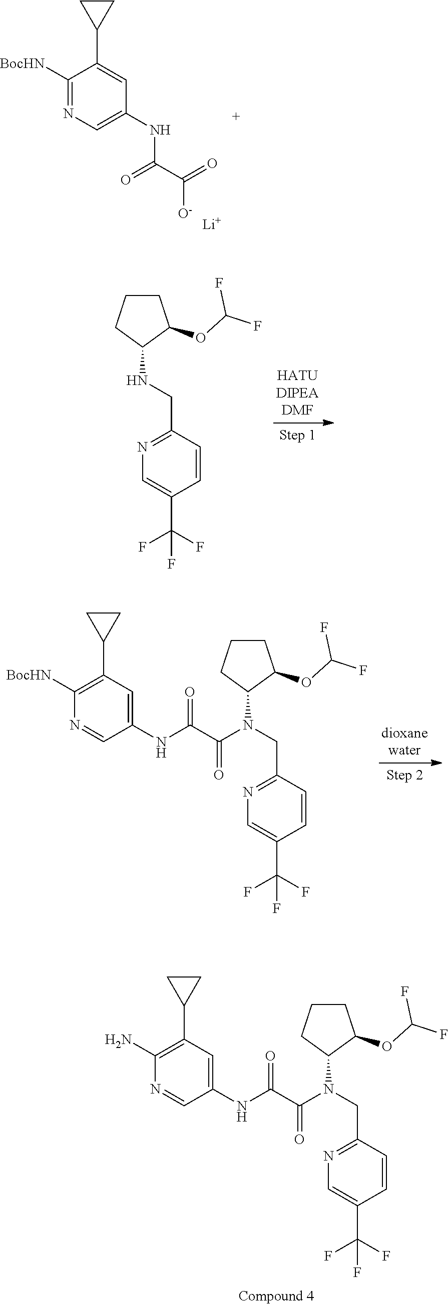 [SMILES: CC(C)(C)OC(=O)Nc1ncc(NC(=O)C(=O)[O-])cc1C1CC1 ; [Li+] ; FC(F)O[C@@H]1CCC[C@H]1NCc1ccc(C(F)(F)F)cn1 ; CC(C)(C)OC(=O)Nc1ncc(NC(=O)C(=O)N(Cc2ccc(C(F)(F)F)cn2)[C@@H]2CCC[C@H]2OC(F)F)cc1C1CC1 ; Nc1ncc(NC(=O)C(=O)N(Cc2ccc(C(F)(F)F)cn2)[C@@H]2CCC[C@H]2OC(F)F)cc1C1CC1]

The synthesis of (1R,2R)-2-(difluoromethoxy)-N-((5-(trifluoromethyl)pyridin-2-yl)methyl)cyclopentan-1-amine is described in Intermediate 3.

### Step 1: tert-butyl(3-cyclopropyl-5-(2-(((1R,2R)-2-(difluoromethoxy)cyclopentyl) ((5-(trifluoromethyl)pyridin-2-yl)methyl)amino)-2-oxoacetamido)pyridin-2-yl)carbamate

DIPEA (276.45 mg, 2.14 mmol, 372.57 μL) was added to the solution of respective 2-[[6-(tert-butoxycarbonylamino)-5-cyclopropyl-3-pyridyl]amino]-2-oxo-acetate (0.2 g, 611.13 μmol, Li+) and (1R,2R)-2-(difluoromethoxy)-N-[5-(trifluoromethyl)-2-pyridyl]methyl]cyclopentanamine (189.61 mg, 611.13 μmol) in DMF (9.63 mL). The resulting mixture was stirred for 5 min followed by the addition of HATU (255.61 mg, 672.25 μmol). Then, the reaction mixture was stirred overnight at rt. After the completion of the reaction, monitored by LCMS, the resulting suspension was concentrated under reduced pressure. It was dissolved in EtOAc (20 ml) and washed with water (5 ml), brine (5 ml), dried over Na₂SO₄ and evaporated. The obtained solid was subjected to HPLC (Waters Sunfire C18 20*100 5 mkm column and MeOH+NH3 as an eluent mixture) to afford tert-butyl(3-cyclopropyl-5-(2-(((1R,2R)-2-(difluoromethoxy)cyclopentyl) ((5-(trifluoromethyl)pyridin-2-yl)methyl)amino)-2-oxoacetamido)pyridin-2-yl)carbamate (73.9 mg, 120.44 μmol, 19.71% yield).

LCMS (ESI): [M+H]⁺m/z: calcd 614.3; found 614.2; Rt=4.120 min.

### Step 2: The synthesis of N1-(6-amino-5-cyclopropylpyridin-3-yl)-N2-((1R,2R)-2-(difluoromethoxy)cyclopentyl)-N2-((5-(trifluoromethyl)pyridin-2-yl)methyl)oxalamide

tert-butyl N-[3-cyclopropyl-5-[2-oxo-2-[(1R,2R)-2-(difluoromethoxy)cyclopentyl]-[5-(trifluoromethyl)-2-pyridyl]methyl]amino]acetyl]amino]-2-pyridyl]carbamate (73.9 mg, 120.44 μmol) was dissolved in dioxane (1 mL) and water (5 mL) mixture. Then reaction mixture was stirred for 16 hr at 95° C. After the completion of the reaction, monitored by LCMS, the resulting suspension was concentrated under reduced pressure. The obtained solid was subjected to HPLC (Waters Sunfire C18 20*100 5 mkm column and MeCN as an eluent mixture) to afford N¹-(6-amino-5-cyclopropylpyridin-3-yl)-N2-((1R,2R)-2-(difluoromethoxy)cyclopentyl)-N2-((5-(trifluoromethyl)pyridin-2-yl)methyl)oxalamide (19.8 mg, 38.56 μmol, 32.02% yield) as a beige solid.

¹H NMR (DMSO-d6, 600 MHz): δ (ppm) 0.37-0.89 (m, 4H), 1.61-2.06 (m, 7H), 4.28-4.98 (m, 4H), 5.72-5.77 (m, 2H), 6.43-6.73 (m, 1H), 7.16 (m, 1H), 7.48-7.64 (m, 1H), 7.90-8.07 (m, 1H), 8.14-8.19 (m, 1H), 8.85-8.90 (m, 1H), 10.26-10.41 (m, 1H).

LCMS (ESI): [M+H]⁺m/z: calcd 514.2; found 514.2; Rt=3.110 min.

### Example 25. Synthesis of (R)—N¹-(2,3-dihydro-1H-inden-1-yl)-N2-(1H-pyrazolo[4,3-c]pyridin-7-yl)-N1-((5-(trifluoromethyl)pyridin-2-yl)methyl)oxalamide (Compound 26)

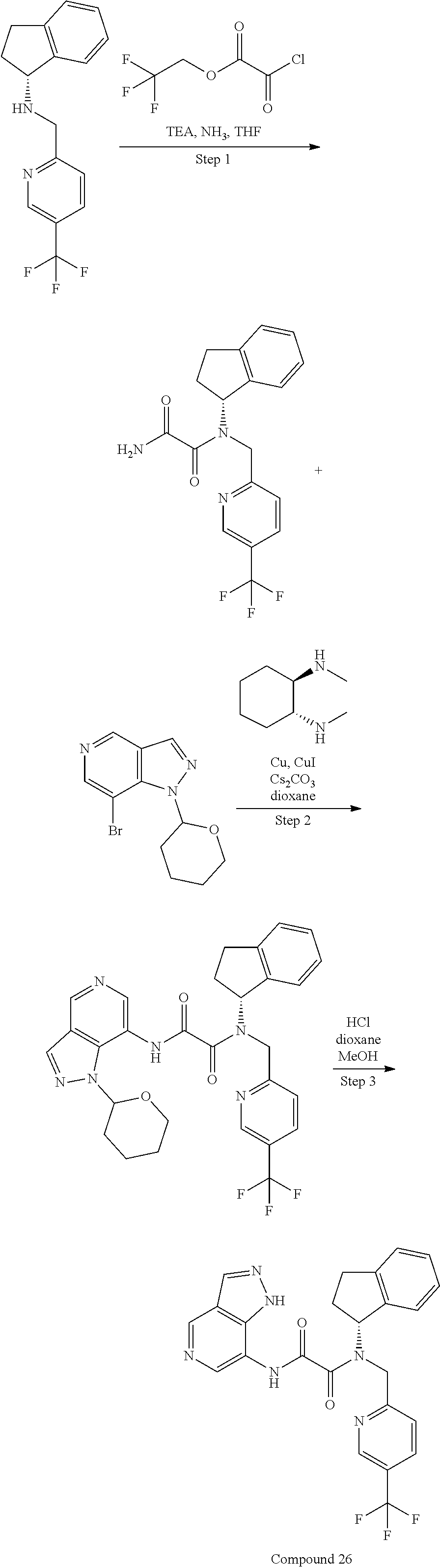

The synthesis of (1R)—N-[[5-(trifluoromethyl)-2-pyridyl]methyl]indan-1-amine is described in Intermediate 2.

### Step 1: N′—[(1R)-indan-1-yl]-N′-[5-(trifluoromethyl)-2-pyridyl]methyl]oxamide

To a solution of (1R)—N-[5-(trifluoromethyl)-2-pyridyl]methyl]indan-1-amine (2.3 g, 7.87 mmol) and TEA (1.59 g, 15.74 mmol, 2.19 mL) in THF (18.40 mL) was added 2,2,2-trifluoroethyl 2-chloro-2-oxo-acetate (2.25 g, 11.80 mmol) dropwise at 0° C. under argon. The reaction mixture was then stirred for 12 hr at r.t. Then ammonia (155.05 mg, 9.10 mmol) was bubbled through for 10 min at 0° C. The reaction mixture was then stirred for 12 hr at r.t. The reaction mixture was filtered off and the filtrate was evaporated in vacuo to give N′—[(1R)-indan-1-yl]-N′-[5-(trifluoromethyl)-2-pyridyl]methyl]oxamide (2 g, 5.50 mmol, 69.96% yield).

LCMS (ESI): [M+H]⁺m/z: calcd 364.1; found 364.0; Rt=1.286 min.

### Step 2: N′—[(1R)-indan-1-yl]-N-(1-tetrahydropyran-2-ylpyrazolo[4,3-c]pyridin-7-yl)-N′-[[5-(trifluoromethyl)-2-pyridyl]methyl]oxamide

A mixture of N′—[(1R)-indan-1-yl]-N′-[5-(trifluoromethyl)-2-pyridyl]methyl]oxamide (380 mg, 836.70 μmol), 7-bromo-1-tetrahydropyran-2-yl-pyrazolo[4,3-c]pyridine (250 mg, 886.10 μmol), copper (5 mg, 78.68 μmol), Copper (I) iodide (100 mg, 525.07 μmol, 17.79 μL), cesium carbonate (599.75 mg, 1.84 mmol) and rac-(1R,2R)—N1,N2-dimethylcyclohexane-1,2-diamine (100 mg, 703.04 μmol) in 1,4-dioxane (6.00 mL) was stirred in a sealed vial under argon at 105° C. for 48 hr. The resulting mixture was cooled down and submitted to reverse phase HPLC (column: Chromatorex 18 SMB100-5T 100×19 mm 5 μm; mobile phase: 40-40-75% 0-1-6 min H₂O/Acetonitrile, flow rate: 30 ml/min (loading pump 4 ml/min acetonitrile) to afford N′—[(1R)-indan-1-yl]-N-(1-tetrahydropyran-2-ylpyrazolo[4,3-c]pyridin-7-yl)-N′-[[5-(trifluoromethyl)-2-pyridyl]methyl]oxamide (62 mg, 109.82 μmol, 13.13% yield) as a light-brown solid.

LCMS (ESI): [M+H]⁺m/z: calcd 565.3; found 565.2; Rt=1.021 min.

### Step 3: The synthesis of (R)—N¹-(2,3-dihydro-1H-inden-1-yl)-N2-(1H-pyrazolo[4,3-c]pyridin-7-yl)-N1-((5-(trifluoromethyl)pyridin-2-yl)methyl)oxalamide

Hydrogen chloride solution 4.0M in dioxane (1.63 g, 6.20 mmol, 2.03 mL, 13.9% purity) was added to a stirred solution of N′—[(1R) indan-1-yl]-N-(1-tetrahydropyran-2-ylpyrazolo[4,3-c]pyridin-7-yl)-N′-[5-(trifluoromethyl)-2-pyridyl]methyl]oxamide (62 mg, 109.82 μmol) in methanol (4.91 mL) at 25° C. The resulting mixture was stirred in a sealed vial at 25° C. for 15 hr, and then concentrated to dryness in vacuo. The residue was submitted to reverse phase HPLC (Chromatorex 18 SMB100-5T 100×19 mm 5 μm; mobile phase: 25-50% 0-1-5 min H₂O/ACN/0.1% FA; flow rate: 30 ml/min (loading pump 4 ml/min acetonitrile) to afford (R)—N¹-(2,3-dihydro-1H-inden-1-yl)-N2-(1H-pyrazolo[4,3-c]pyridin-7-yl)-N1-((5-(trifluoromethyl)pyridin-2-yl)methyl)oxalamide (29 mg, 60.36 μmol, 54.96% yield) as a light-yellow solid.

¹H NMR (DMSO-d6, 600 MHz): δ (ppm) 1.91-2.36 (m, 2H), 2.80-2.94 (m, 2H), 4.22-5.10 (m, 2H), 6.05-6.12 (m, 1H), 7.12-7.24 (m, 3H), 7.37-7.55 (m, 2H), 7.99-8.48 (m, 3H), 8.78-8.96 (m, 2H), 10.98-11.25 (m, 1H), 13.07-13.13 (m, 1H).

LCMS (ESI): [M+H]⁺m/z: calcd 481.2; found 481.2; Rt=3.076 min.

### Example 26. Synthesis of N-(4-amino-1H-pyrazolo[4,3-c]pyridin-7-yl)-N′-benzyl-N′-(pyrimidin-2-ylmethyl)oxamide (Compound 14)

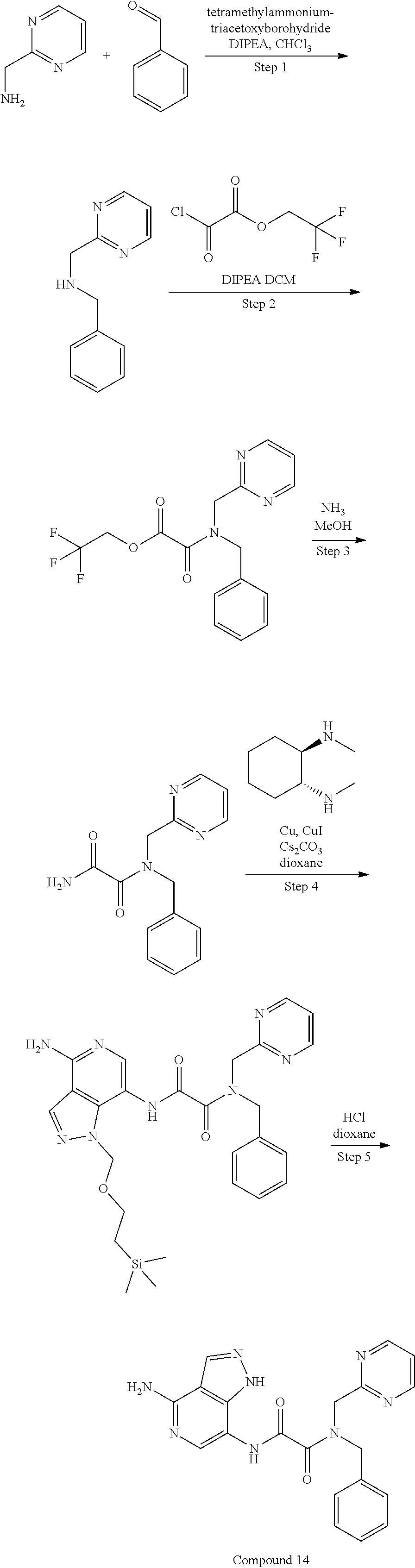

### Step 1: The synthesis of N-(pyrimidin-2-ylmethyl)-1-[5-(trifluoromethyl)-2-pyridyl]methanamine

pyrimidin-2-ylmethanamine (8 g, 54.95 mmol, HCl), benzaldehyde (5.83 g, 54.95 mmol),DIPEA (10.65 g, 82.42 mmol, 14.36 mL) were mixed in Chloroform (107.44 mL) followed by portionwise addition of tetramethylammonium triacetoxyborohydride (28.91 g, 109.90 mmol). Reaction mixture was stirred at RT overnight, washed with NaHCO₃ solution and concentrated under reduced pressure. The residue was submitted to flash column chromatography to afford 1-phenyl-N-(pyrimidin-2-ylmethyl)methanamine (3.5 g, 17.57 mmol, 31.97% yield).

Chromatography: Interchim; 120 g SiO₂,MTBE-MeOH from 0~100%, flow rate=70 mL/min, cv=7.2.

LCMS (ESI): [M+H]⁺m/z: calcd 200.12; found 200.2; Rt=0.689 min.

### Step 2: The synthesis of 2,2,2-trifluoroethyl 2-[benzyl(pyrimidin-2-ylmethyl)amino]-2-oxo-acetate

2,2,2-trifluoroethyl 2-chloro-2-oxo-acetate (956.10 mg, 5.02 mmol) was added dropwise to an ice bath cold stirred solution of 1-phenyl-N-(pyrimidin-2-ylmethyl)methanamine (1 g, 5.02 mmol) and DIPEA (843.22 mg, 6.52 mmol, 1.14 mL) in DCM (25 mL). The reaction mixture was stirred overnight and concentrated on rotary evaporator. Upon completion, the reaction mixture was washed with water. The organic layer was separated, dried over Na₂SO₄ and concentrated under reduced pressure to afford 2,2,2-trifluoroethyl 2-[benzyl(pyrimidin-2-ylmethyl)amino]-2-oxo-acetate (1.5 g, crude) which was used in the next step without purification.

LCMS (ESI): [M+H]⁺m/z: calcd 354.1; found 354.2; Rt=1.149 min.

### Step 3: The synthesis of N′-benzyl-N′-(pyrimidin-2-ylmethyl)oxamide

2,2,2-trifluoroethyl 2-[benzyl(pyrimidin-2-ylmethyl)amino]-2-oxo-acetate (1.5 g, 4.25 mmol) was dissolved in methanol/NH3 solution and stirred overnight. Upon completion, the reaction mixture was concentrated under reduced pressure to afford N′-benzyl-N′-(pyrimidin-2-ylmethyl)oxamide (1 g, crude) which was used in the next step without purification.

LCMS (ESI): [M+H]⁺m/z: calcd 271.1; found 271.0; Rt=0.757 min.

### Step 4: The synthesis of N-[4-amino-1-(2-trimethylsilylethoxymethyl)pyrazolo[4,3-c]pyridin-7-yl]-N′-benzyl-N′-(pyrimidin-2-ylmethyl)oxamide)

N′-benzyl-N′-(pyrimidin-2-ylmethyl)oxamide (0.3 g, 1.11 mmol), 7-bromo-1-(2-trimethylsilylethoxymethyl)pyrazolo[4,3-c]pyridin-4-amine (381.03 mg, 1.11 mmol), copper (5.30 mg, 83.34 μmol), Copper (I) iodide (127.11 mg, 667.42 μmol, 22.62 μL), (1S,2S)-N,N′-Bis -methyl-1,2-cyclohexane-diamine (157.88 mg, 1.11 mmol, 175.03 μL) were mixed in dioxane (6 mL). The reaction mixture was stirred under Ar atmosphere at 100° C. for 48 hr. Upon completion, the mixture concentrated under reduced pressure, treated with DMSO and filtered. The filtrate was submitted to HPLC to afford N-[4-amino-1-(2-trimethylsilylethoxymethyl)pyrazolo[4,3-c]pyridin-7-yl]-N′-benzyl-N′-(pyrimidin-2-ylmethyl)oxamide (0.054 g, 101.38 μmol, 9.13% yield).

HPLC: 2-10 min 20-40% acetonitrile+fa flo.

LCMS (ESI): [M+1]⁺m/z: calcd 533.3; found 533.0; Rt=1.082 min.

### Step 5: The synthesis of N-(4-amino-1H-pyrazolo[4,3-c]pyridin-7-yl)-N′-benzyl-N′-(pyrimidin-2-ylmethyl)oxamide

N-[4-amino-1-(2-trimethylsilylethoxymethyl)pyrazolo[4,3-c]pyridin-7-yl]-N′-benzyl-N′-(pyrimidin-2-ylmethyl)oxamide (0.054 g, 101.38 μmol) was dissolved in HCl/dioxane solution (13%, 2 ml) and stirred at 25° C. for 2 hr. Upon completion, the reaction mixture was concentrated under reduced pressure and residue was purified by reverse phase HPLC to afford N-(4-amino-1H-pyrazolo[4,3-c]pyridin-7-yl)-N′-benzyl-N′-(pyrimidin-2-ylmethyl)oxamide (0.016 g, 39.76 μmol, 39.22% yield).

HPLC: 2-10 min 0-70% acn+FA 30 ml/min.

¹H NMR (600 MHz, dmso) 8 4.53-4.75 (m, 2H), 4.87-5.12 (m, 2H), 6.61-6.78 (m, 2H), 7.25-7.29 (m, 1H), 7.31-7.35 (m, 3H), 7.35-7.44 (m, 2H), 7.57-7.72 (m, 1H), 8.13-8.23 (m, 1H), 8.71-8.82 (m, 2H), 10.34-10.64 (m, 1H), 12.79 (br s, 1H).

LCMS (ESI): [M+1]⁺m/z: calcd 403.2; found 403.2; Rt=1.473 min.

### Example 27. Synthesis of N1-(6-amino-5-methylpyridin-3-yl)-N2-((1R,2R)-2-(difluoromethoxy)cyclopentyl)-N2-((5-(trifluoromethyl)pyridin-2-yl)methyl)oxalamide (Compound 19)

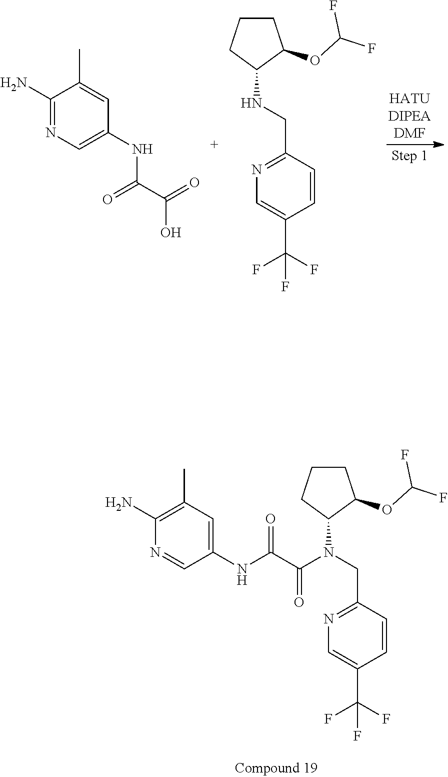 [SMILES: Cc1cc(NC(=O)C(=O)O)cnc1N ; FC(F)O[C@@H]1CCC[C@H]1NCc1ccc(C(F)(F)F)cn1 ; Cc1cc(NC(=O)C(=O)N(Cc2ccc(C(F)(F)F)cn2)[C@@H]2CCC[C@H]2OC(F)F)cnc1N]

The synthesis of (1R,2R)-2-(difluoromethoxy)-N-((5-(trifluoromethyl)pyridin-2-yl)methyl)cyclopentan-1-amine is described in Intermediate 3.

### Step 1. The synthesis of N1-(6-amino-5-methylpyridin-3-yl)-N2-((1R,2R)-2-(difluoromethoxy)cyclopentyl)-N2-((5-(trifluoromethyl)pyridin-2-yl)methyl)oxalamide

DIPEA (162.24 mg, 1.26 mmol, 218.65 μL) was added to the solution of respective 2-[(6-amino-5-methyl-3-pyridyl)amino]-2-oxo-acetic acid (0.07 g, 358.65 μmol) and (1R,2R)-2-(difluoromethoxy)-N-[5-(trifluoromethyl)-2-pyridyl]methyl]cyclopentanamine (111.28 mg, 358.65 μmol) in DMF (9.78 mL). The resulting mixture was stirred for 5 min followed by the addition of HATU (150.01 mg, 394.52 μmol). Then, the reaction mixture was stirred overnight at rt. After the completion of the reaction, monitored by LCMS, the resulting suspension was concentrated under reduced pressure. It was dissolved in EtOAc (20 ml) and washed with water (5 ml), brine (5 ml), dried over Na₂SO₄ and evaporated. The obtained solid was subjected to HPLC (Waters Sunfire C18 20*100 5 mkm column and MeOH+NH3 as an eluent mixture) to afford N1-(6-amino-5-methylpyridin-3-yl)-N2-((1R,2R)-2-(difluoromethoxy)cyclopentyl)-N2-((5-(trifluoromethyl)pyridin-2-yl)methyl)oxalamide (11.9 mg, 24.41 μmol, 6.81% yield) as a yellow solid.

¹H NMR (DMSO-d6, 600 MHz): δ (ppm) 1.64 (m, 3H), 1.76 (m, 2H), 1.99 (m, 4H), 4.62 (m, 3H), 4.96 (m, 1H), 5.62 (m, 2H), 6.58 (m, 1H), 7.49 (m, 2H), 7.95 (m, 1H), 8.17 (m, 1H), 8.88 (m, 1H), 10.37 (m, 1H).

LCMS (ESI): [M+H]⁺m/z: calcd 488.2; found 488.2; Rt=2.950 min.

### Example 28. Synthesis of N-(5-carbamoyl-6-methoxy-3-pyridyl)-N′-(4-methyltetralin-1-yl)-N′-[5-(trifluoromethyl)-2-pyridyl]methyl]oxamide (Compound 33)

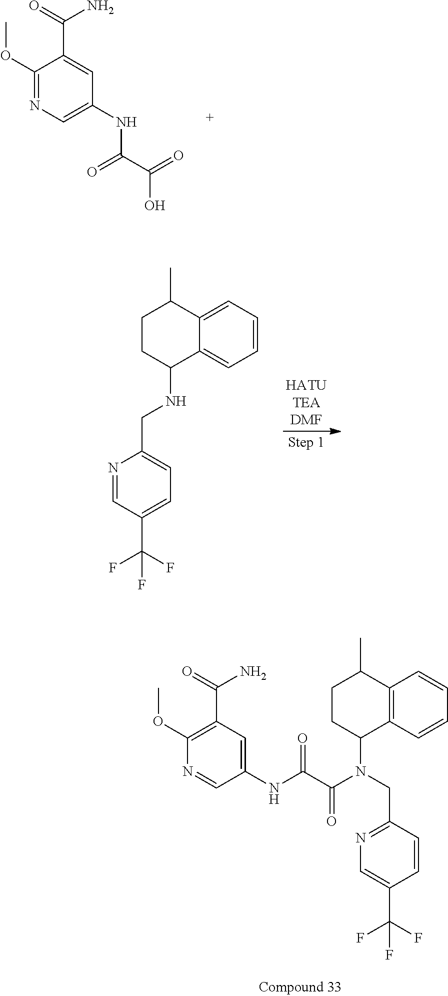 [SMILES: COc1ncc(NC(=O)C(=O)O)cc1C(N)=O ; CC1CCC(NCc2ccc(C(F)(F)F)cn2)c2ccccc21 ; COc1ncc(NC(=O)C(=O)N(Cc2ccc(C(F)(F)F)cn2)C2CCC(C)c3ccccc32)cc1C(N)=O]

The synthesis of (rac)-4-methyl-N-((5-(trifluoromethyl)pyridin-2-yl)methyl)-1,2,3,4-tetrahydronaphthalen-1-amine is described in Intermediate 4.

### Step 1. The synthesis of N-(5-carbamoyl-6-methoxy-3-pyridyl)-N′-(4-methyltetralin-1-yl)-N′-[[5-(trifluoromethyl)-2-pyridyl]methyl]oxamide

To the solution of (rac)-4-methyl-N-[5-(trifluoromethyl)-2-pyridyl]methyl]tetralin-1-amine (248.44 mg, 775.51 μmol), 2-[(5-carbamoyl-6-methoxy-3-pyridyl)amino]-2-oxo-acetic acid (185.49 mg, 775.51 μmol) and Triethylamine (235.42 mg, 2.33 mmol, 324.27 μL) in DMF (3 mL) HATU (324.36 mg, 853.06 μmol) was added portionwise. Mixture was stirred at 25° C. for 2 hr. The reaction mixture was submitted for HPLC (SYSTEM 30-30-80% 0-1-5 min H₂O/ACN/0.1% NH₄OH, flow: 30 ml/min (loading pump 4 ml/min acetonitrile) target mass 541 column: XBridge BEH C18 100×19 mm, 5 μm) to give N-(5-carbamoyl-6-methoxy-3-pyridyl)-N′-(4-methyltetralin-1-yl)-N′-[[5-(trifluoromethyl)-2-pyridyl]methyl]oxamide (16 mg, 29.55 μmol, 3.81% yield) as a white solid.

¹H NMR (DMSO-d6, 600 MHz): δ (ppm) 1.17-1.23 (m, 3H), 1.39-2.18 (m, 4H), 2.80-2.89 (m, 1H), 3.92-3.99 (m, 3H), 4.46-5.12 (m, 3H), 5.30-5.79 (m, 2H), 7.12-7.34 (m, 3H), 7.54-7.62 (m, 1H), 7.67-7.72 (m, 1H), 8.04-8.58 (m, 3H), 8.76-8.86 (m, 1H), 10.89-11.20 (m, 1H).

LCMS (ESI): [M+H]⁺m/z: calcd 542.2; found 542.0; Rt=3.902 min.

### Example 29. Synthesis of (R)—N¹-(1-cyclopropyl-2-methoxyethyl)-N2-(imidazo[1,2-a]pyridin-7-yl)-N1-((5-(trifluoromethyl)pyridin-2-yl)methyl)oxalamide (Compound 31)

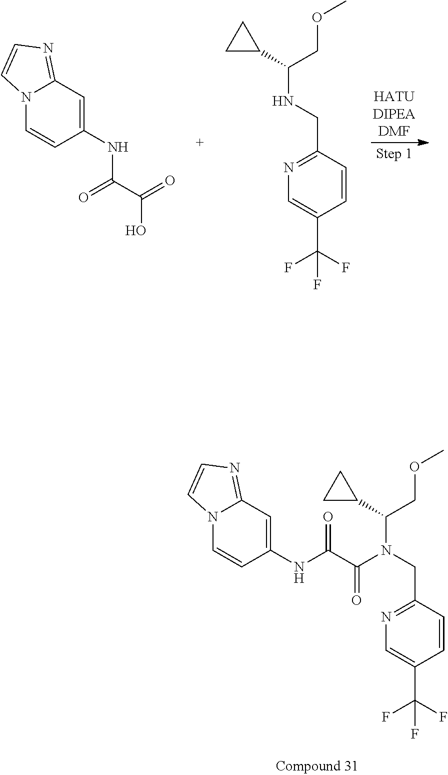 [SMILES: O=C(O)C(=O)Nc1ccn2ccnc2c1 ; COC[C@H](NCc1ccc(C(F)(F)F)cn1)C1CC1 ; COC[C@@H](C1CC1)N(Cc1ccc(C(F)(F)F)cn1)C(=O)C(=O)Nc1ccn2ccnc2c1]

The synthesis of (R)-1-cyclopropyl-2-methoxy-N-((5-(trifluoromethyl)pyridin-2-yl)methyl) ethan-1-amine is described in Intermediate 1.

### Step 1. The synthesis of (R)—N¹-(1-cyclopropyl-2-methoxyethyl)-N2-(imidazo[1,2-a]pyridin-7-yl)-N1-((5-(trifluoromethyl)pyridin-2-yl)methyl)oxalamide

To a mixture of (1R)-1-cyclopropyl-2-methoxy-N-[[5-(trifluoromethyl)-2-pyridyl]methyl]ethanamine (0.1 g, 364.59 μmol), 2-(imidazo[1,2-a]pyridin-7-ylamino)-2-oxo-acetic acid (74.80 mg, 364.59 μmol) and TEA (184.46 mg, 1.82 mmol, 254.08 μL) in DMF (3 mL),HATU (166.35 mg, 437.51 μmol) was added in one portion. The resulting mixture was allowed to stir at 24° C. for 3 hr. Upon completion, DMF was evaporated, the residue was subjected by HPLC (column: XBridge BEH C18 100×19 mm 5 μm; mobile phase: 30-60%, 0-1-6 min H₂O/MeCN/0.1% NH₄OH, flow rate: 30 ml/min (loading pump 4 ml/min MeCN)) to afford (R)—N¹-(1-cyclopropyl-2-methoxyethyl)-N2-(imidazo[1,2-a]pyridin-7-yl)-N1-((5-(trifluoromethyl)pyridin-2-yl)methyl)oxalamide (37.4 mg, 81.05 μmol, 22.23% yield) as a light-yellow solid.

¹H NMR (DMSO-d6, 600 MHz): δ (ppm) 0.12-0.56 (m, 4H), 0.94-0.99 (m, 1H), 3.09-3.14 (m, 3H), 3.43-383 (m, 2H), 4.77-5.12 (m, 2H), 7.05-7.12 (m, 1H), 7.45-7.49 (m, 1H), 7.59-7.60 (m, 1H), 7.76-7.98 (m, 3H), 8.15-8.24 (m, 1H), 8.40-8.47 (m, 1H), 8.79-8.90 (m, 1H), 10.86-10.97 (m, 1H).

LCMS (ESI): [M+H]⁺m/z: calcd 462.2; found 462.2; Rt=1.990 min.

### Example 30. Synthesis of N-(4-amino-1H-pyrazolo[4,3-c]pyridin-7-yl)-N′-[[4-(trifluoromethyl)phenyl]methyl]-N′-[[6-(trifluoromethyl)-3-pyridyl]methyl]oxamide (Compound 28)

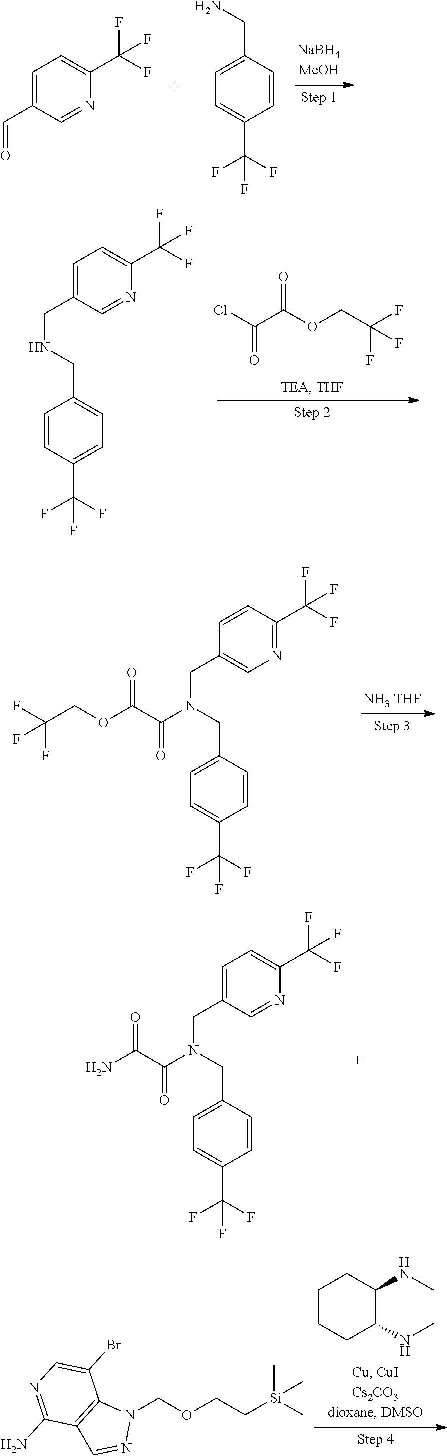

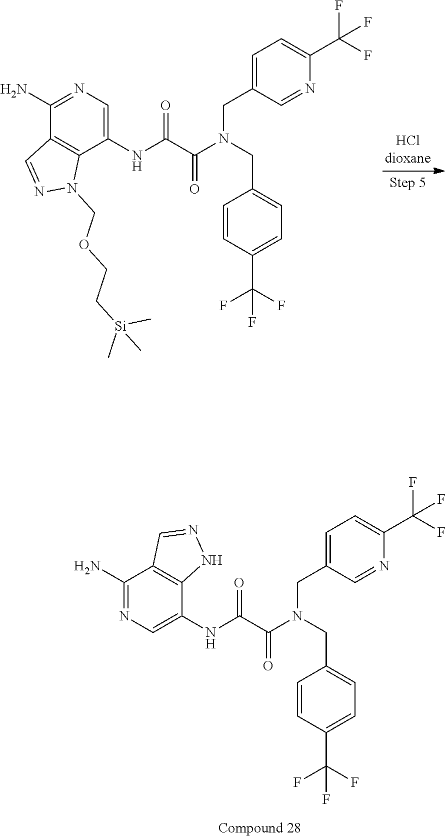 [SMILES: C[Si](C)(C)CCOCn1ncc2c(N)ncc(NC(=O)C(=O)N(Cc3ccc(C(F)(F)F)cc3)Cc3ccc(C(F)(F)F)nc3)c21 ; Nc1ncc(NC(=O)C(=O)N(Cc2ccc(C(F)(F)F)cc2)Cc2ccc(C(F)(F)F)nc2)c2[nH]ncc12]

### Step 1:1-[4-(trifluoromethyl)phenyl]-N-[[6-(trifluoromethyl)-3-pyridyl]methyl]methanamine

a6-(trifluoromethyl)pyridine-3-carbaldehyde (1 g, 5.71 mmol) was added to a stirred solution of [4-(trifluoromethyl)phenyl]methanamine (1.00 g, 5.71 mmol, 813.87 μL) in Methanol (29.19 mL) and stirred at 50° C. for 2 hr. Reaction mixture was concentrated, mixed with NaOH (15% 5 mL), extracted with DCM (2×15 mL), combined extract dried over Na₂SO₄ and evaporated in vacuo to give crude product which was purified by flash chromatography to give pure 1-[4-(trifluoromethyl)phenyl]-N-[[6-(trifluoromethyl)-3-pyridyl]methyl]methanamine (0.65 g, 1.94 mmol, 34.05% yield).

LCMS (ESI): [M+H]⁺m/z: calcd 335.1; found 335.0; Rt=0.783 min.

### Step 2:2,2,2-trifluoroethyl 2-oxo-2-[[4-(trifluoromethyl)phenyl]methyl-[[6-(trifluoromethyl)-3-pyridyl]methyl]amino]acetate

2,2,2-trifluoroethyl 2-chloro-2-oxo-acetate (388.98 mg, 2.04 mmol) was added dropwise to a stirred solution of 1-[4-(trifluoromethyl)phenyl]-N-[[6-(trifluoromethyl)-3-pyridyl]methyl]methanamine (650.00 mg, 1.94 mmol) and TEA (393.55 mg, 3.89 mmol, 542.08 μL) in THF (20 mL) at 0° C., stirred for 1 hr at 0° C. Reaction mixture was used in the next step.

LCMS (ESI): [M+H]⁺m/z: calcd 489.1; found 489.2; Rt=1.411 min.

### Step 3: N′-benzyl-N′-(pyrimidin-2-ylmethyl)oxamide

Ammonia (661.96 mg, 38.87 mmol) was bubbled trough a reaction mixture at 0° C., stirred for 1 hr at 0° C. and 2 hr at 25° C. Reaction mixture was filtered, solid washed with THF (2×5 mL), filtrate evaporated in vacuo to give N′-[4-(trifluoromethyl)phenyl]methyl]-N′-[6-(trifluoromethyl)-3-pyridyl]methyl]oxamide (0.75 g, 1.85 mmol, 95.22% yield).

LCMS (ESI): [M+H]⁺m/z: calcd 406.1; found 406.0; Rt=1.056 min.

### Step 4: N-[4-amino-1-(2-trimethylsilylethoxymethyl)pyrazolo[4,3-c]pyridin-7-yl]-N′-[[4-(trifluoromethyl)phenyl]methyl]-N′-[[6-(trifluoromethyl)-3-pyridyl]methyl]oxamide

Copper (1.96 mg, 30.84 μmol), Copper (I) iodide (58.74 mg, 308.42 μmol, 10.45 μL), cesium carbonate (602.93 mg, 1.85 mmol) was added to a stirred solution of N′-[[4-(trifluoromethyl)phenyl]methyl]-N′-[6-(trifluoromethyl)-3-pyridyl]methyl]oxamide (0.25 g, 616.84 μmol), 7-bromo-1-(2-trimethylsilylethoxymethyl)pyrazolo[4,3-c]pyridin-4-amine (232.93 mg, 678.52 μmol), rac-(1R,2R)—N1,N2-dimethylcyclohexane-1,2-diamine (171.09 mg, 1.20 mmol) in DMSO(1 mL)/1,4-dioxane (5 mL) under Ar atmosphere and stirred at 110° C. for 48 hr in closed vial. Reaction mixture was filtered, solid washed with dioxane (2×3 mL), filtrate concentrated to give crude N-[4-amino-1-(2-trimethylsilylethoxymethyl)pyrazolo[4,3-c]pyridin-7-yl]-N′-[4-(trifluoromethyl)phenyl]methyl]-N′-[6-(trifluoromethyl)-3-pyridyl]methyl]oxamide (0.4 g, 599.09 μmol, 97.12% yield).

LCMS (ESI): [M+H]⁺m/z: calcd 668.2; found 668.2; Rt=3.961 min.

### Step 5: The synthesis of N-(4-amino-1H-pyrazolo[4,3-c]pyridin-7-yl)-N′-[[4-(trifluoromethyl)phenyl]methyl]-N′-[[6-(trifluoromethyl)-3-pyridyl]methyl]oxamide

Hydrogen chloride solution 4.0M in dioxane (1.64 g, 44.93 mmol, 2.05 mL) was added to a stirred solution of N-[4-amino-1-(2-trimethylsilylethoxymethyl)pyrazolo[4,3-c]pyridin-7-yl]-N′-[4-(trifluoromethyl)phenyl]methyl]-N′-[6-(trifluoromethyl)-3-pyridyl]methyl]oxamide (0.4 g, 599.09 μmol) in Methanol (2 mL) and stirred at 20° C. for 48 hr. Reaction mixture was concentrated and purified by HPLC (column: XBridge BEH C18 100×19 mm, 5 μm; 30-80% 0-6 min H₂O/MeOH/0.1% NH₄OH, flow rate: 30 ml/min) to give two crude fractions.

Second HPLC purification (column: XBridge C18 100×19 mm, 5 μm; 35-85% 0-5 min H₂O/MeOH/0.1% NH₄OH, flow: 30 ml/min) afford N-(4-amino-1H-pyrazolo[4,3-c]pyridin-7-yl)-N′-[4-(trifluoromethyl)phenyl]methyl]-N′-[6-(trifluoromethyl)-3-pyridyl]methyl]oxamide (35 mg, 65.13 μmol, 10.87% yield).

¹H NMR (600 MHz, dmso) 8 4.54-5.03 (m, 4H), 6.69 (s, 2H), 7.45-7.49 (m, 1H), 7.57-7.65 (m, 4H), 7.76-7.83 (m, 1H), 7.89-8.05 (m, 1H), 8.13-8.21 (m, 1H), 8.60-8.74 (m, 1H), 9.88-10.74 (m, 1H), 12.77-13.39 (m, 1H).

LCMS (ESI): [M+H]⁺m/z: calcd 538.2; found 538.0; Rt=0.914 min.

### Example 31. Synthesis of N-imidazo[1,2-a]pyridin-7-yl-N′-(4-methyltetralin-1-yl)-N′-[[5-(trifluoromethyl)-2-pyridyl]methyl]oxamide (Compound 12)

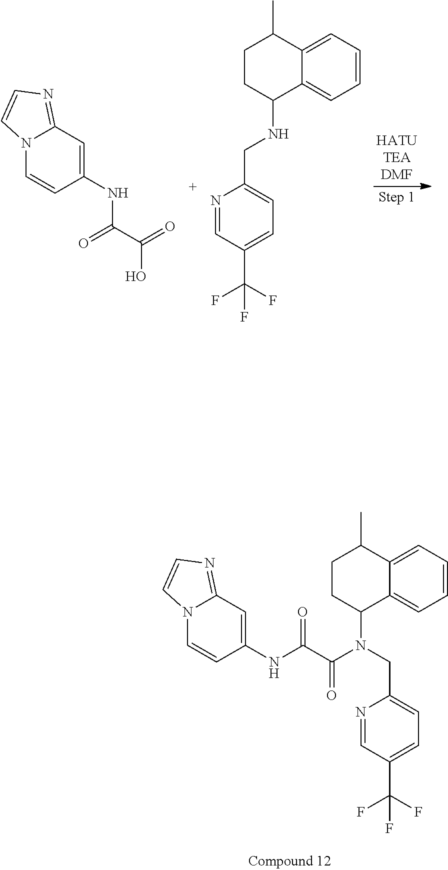

The synthesis of (rac)-4-methyl-N-((5-(trifluoromethyl)pyridin-2-yl)methyl)-1,2,3,4-tetrahydronaphthalen-1-amine is described in Intermediate 4.

### Step 1. The synthesis of N-imidazo[1,2-a]pyridin-7-yl-N′-(4-methyltetralin-1-yl)-N′-[[5-(trifluoromethyl)-2-pyridyl]methyl]oxamide

To the solution of (rac)-4-methyl-N-[[5-(trifluoromethyl)-2-pyridyl]methyl]tetralin-1-amine (248.44 mg, 775.51 μmol), 2-(imidazo[1,2-a]pyridin-7-ylamino)-2-oxo-acetic acid (159.11 mg, 775.51 μmol) and Triethylamine (235.42 mg, 2.33 mmol, 324.27 μL) in DMF (3 mL) HATU (324.36 mg, 853.06 μmol) was added portionwise. Mixture was stirred at 25° C. for 2 hr. The reaction mixture was submitted for reverse phase HPLC (SYSTEM 30-30-80% 0-1-5 min H₂O/ACN/0.1% NH₄OH, flow: 30 ml/min (loading pump 4 ml/min acetonitrile) target mass 507 column: XBridge BEH C18 100×19 mm, 5 μm) to give N-imidazo[1,2-a]pyridin-7-yl-N′-(4-methyltetralin-1-yl)-N′-[5-(trifluoromethyl)-2-pyridyl]methyl]oxamide (26 mg, 51.23 μmol, 6.61% yield) as a white solid.

¹H NMR (DMSO-d6, 600 MHz): δ (ppm) 1.17-1.24 (m, 3H), 1.36-2.20 (m, 4H), 2.82-2.91 (m, 1H), 4.48-4.80 (m, 1H), 5.03-5.80 (m, 2H), 7.03-7.24 (m, 3H), 7.31-7.62 (m, 3H), 7.80-8.22 (m, 3H), 8.40-8.49 (m, 1H), 8.76-8.87 (d, 1H), 10.91-11.27 (m, 2H).

LCMS (ESI): [M+H]⁺m/z: calcd 542.2; found 542.2; Rt=3.902 min.

### Example 32. Synthesis of N-(5-carbamoyl-6-methoxy-3-pyridyl)-N′-(2-cyclopropylpropyl)-N′-[[5-(trifluoromethyl)-2-pyridyl]methyl]oxamide (Compound 47)

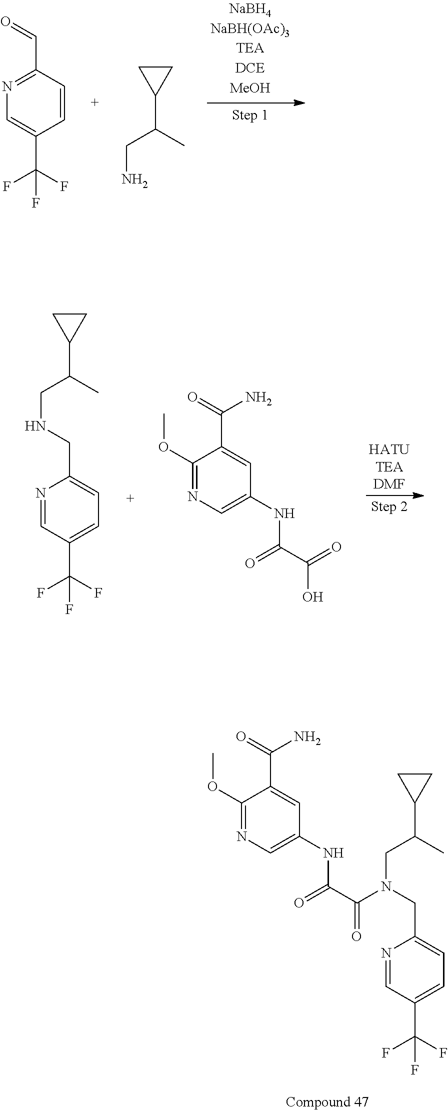

### Step 1: -cyclopropyl-N-[[5-(trifluoromethyl)-2-pyridyl]methyl]propan-1-amine

To a solution of 5-(trifluoromethyl)pyridine-2-carbaldehyde (1.65 g, 9.42 mmol),2-cyclopropylpropan-1-amine (1.92 g, 14.13 mmol, HCl) in DCE (24.72 mL) was added TEA (1.43 g, 14.13 mmol, 1.97 mL) and the resulting solution was cooled to −10° C. Then, Sodium triacetoxyborohydride (2.00 g, 9.42 mmol) was added and the resulting mixture was left to stir at rt for 12 hr. The reaction mixture was quenched with aq sat NaHCO₃, washed with brine, dried and evaporated to dryness to give 2-cyclopropyl-N-[[5-(trifluoromethyl)-2-pyridyl]methyl]propan-1-amine (2.2 g, 8.52 mmol, 90.40% yield) as a yellow oil. After work-up we realized that in the residue 35% of product and 65% of imine intermediate. This was further reduced with Sodium Borohydride (356.49 mg, 9.42 mmol, 331.93 μL) in MeOH (25 mL). The reaction mixture was evaporated, partitioned in mixture EtOAc/water. Water was extracted with EtOAc (10 ml). Combined organics were dried over Na₂SO₄ and evaporated. 2-cyclopropyl-N-[5-(trifluoromethyl)-2-pyridyl]methyl]propan-1-amine (2.2 g, 8.52 mmol, 90.40% yield) was obtained as a light-yellow oil.

### Step 2: The synthesis of N-(5-carbamoyl-6-methoxy-3-pyridyl)-N′-(2-cyclopropylpropyl)-N′-[[5-(trifluoromethyl)-2-pyridyl]methyl]oxamide

2-cyclopropyl-N-[5-(trifluoromethyl)-2-pyridyl]methyl]propan-1-amine (0.1 g, 387.17 μmol), HATU (147.22 mg, 387.17 μmol) and TEA (117.53 mg, 1.16 mmol, 161.89 μL) were mixed in dry DMF (4.89 mL) at rt and the resulting mixture was stirred for 15 min. 2-[(5-carbamoyl-6-methoxy-3-pyridyl)amino]-2-oxo-acetic acid (92.61 mg, 387.17 μmol) was added thereto and the resulting mixture was stirred at rt overnight. The resulting mixture was poured into water, extracted 3 times with EtOAc, combined organics were washed with water, brine, and evaporated. The residue was subjected to HPLC (25-50% 0.5-6.5 min; 30 ml/min water-acetonitrile+NH3 (loading pump 4 ml/min; acetonitrile); column XBridge 19*100 mm). N-(5-carbamoyl-6-methoxy-3-pyridyl)-N′-(2-cyclopropylpropyl)-N′-[5-(trifluoromethyl)-2-pyridyl]methyl]oxamide (102.4 mg, 213.58 μmol, 55.16% yield) was obtained as a white solid.

¹H NMR (DMSO-d6, 600 MHz): δ (ppm) 0.12 (m, 2H), 0.36 (m, 2H), 0.52 (m, 1H), 0.91 (m, 3H), 1.18 (m, 1H), 3.46 (m, 1H), 3.62 (m, 1H), 3.93 (m, 3H), 4.77 (m, 1H), 4.98 (m, 1H), 7.57 (m, 1H), 7.71 (m, 2H), 8.21 (m, 1H), 8.47 (m, 2H), 8.91 (m, 1H), 10.89 (m, 1H).

LCMS (ESI): [M+H]⁺m/z: calcd 480.2; found 480.0; Rt=3.078 min.

### Example 33. Synthesis of (R)—N¹-(5-carbamoylpyridin-3-yl)-N2-(2,3-dihydro-1H-inden-1-yl)-N2-((5-(trifluoromethyl)pyridin-2-yl)methyl)oxalamide (Compound 40)

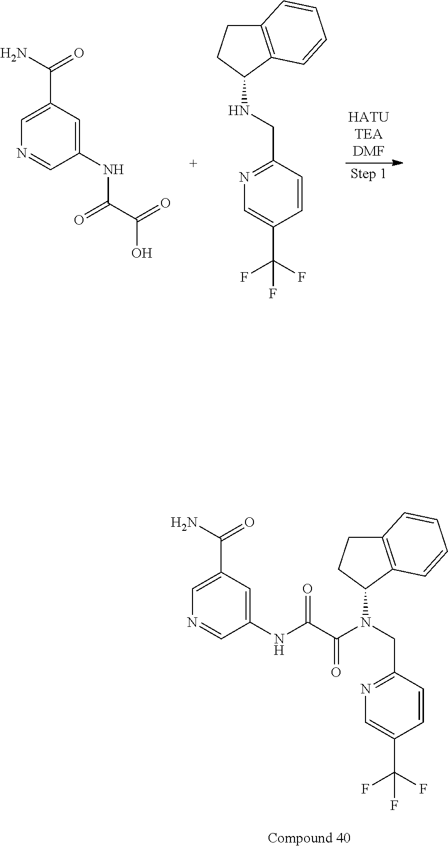 [SMILES: NC(=O)c1cncc(NC(=O)C(=O)O)c1 ; FC(F)(F)c1ccc(CN[C@@H]2CCc3ccccc32)nc1 ; NC(=O)c1cncc(NC(=O)C(=O)N(Cc2ccc(C(F)(F)F)cn2)[C@@H]2CCc3ccccc32)c1]

The synthesis of (R)—N-((5-(trifluoromethyl)pyridin-2-yl)methyl)-2,3-dihydro-1H-inden-1-amine is above is described in Intermediate 2.

### Step 1. The synthesis of (R)—N¹-(5-carbamoylpyridin-3-yl)-N2-(2,3-dihydro-1H-inden-1-yl)-N2-((5-(trifluoromethyl)pyridin-2-yl)methyl)oxalamide

HATU (214.64 mg, 564.49 μmol) was added portionwise at r.t. to a suspension of 2-[(5-carbamoyl-3-pyridyl)amino]-2-oxo-acetic acid (163.86 mg, 667.13 μmol, HCl), (1R)—N-[[5-(trifluoromethyl)-2-pyridyl]methyl]indan-1-amine (150 mg, 513.17 μmol) and TEA (311.57 mg, 3.08 mmol, 429.16 μL) in DMF (6 mL). The clear solution was stirred at 20° C. for 18 hr and the solvents were evaporated in vacuo. The residue was purified by RP HPLC (column: XBridge C18 100×19 mm, 5 μm; 20-70% 0-5 min H₂O/MeOH/0.1% NH₄OH, flow: 30 ml/min) to give (R)-N1-(5-carbamoylpyridin-3-yl)-N2-(2,3-dihydro-1H-inden-1-yl)-N2-((5-(trifluoromethyl)pyridin-2-yl)methyl)oxalamide (10.1 mg, 20.89 μmol, 4.07% yield) as a yellow solid.

¹H NMR (600 MHz, dmso) δ 1.87-2.05 (m, 1H), 2.32-2.41 (m, 1H), 2.71-2.85 (m, 1H), 2.87-2.99 (m, 1H), 3.32-4.28 (m, 1H), 4.56-4.96 (m, 1H), 5.67-6.14 (m, 1H), 7.08-7.21 (m, 2H), 7.22-7.39 (m, 2H), 7.46-7.55 (m, 1H), 7.55-7.67 (m, 1H), 7.98-8.22 (m, 2H), 8.36-8.56 (m, 1H), 8.68-8.99 (m, 3H), 11.01-11.53 (m, 1H).

LCMS (ESI): [M+H]⁺m/z: calcd 484.2; found 484.0; Rt=2.931 min.

### Example 34. Synthesis of N′-(4-methyltetralin-1-yl)-N-(1H-pyrazolo[4,3-c]pyridin-7-yl)-N′-[[5-(trifluoromethyl)-2-pyridyl]methyl]oxamide (Compound 17)

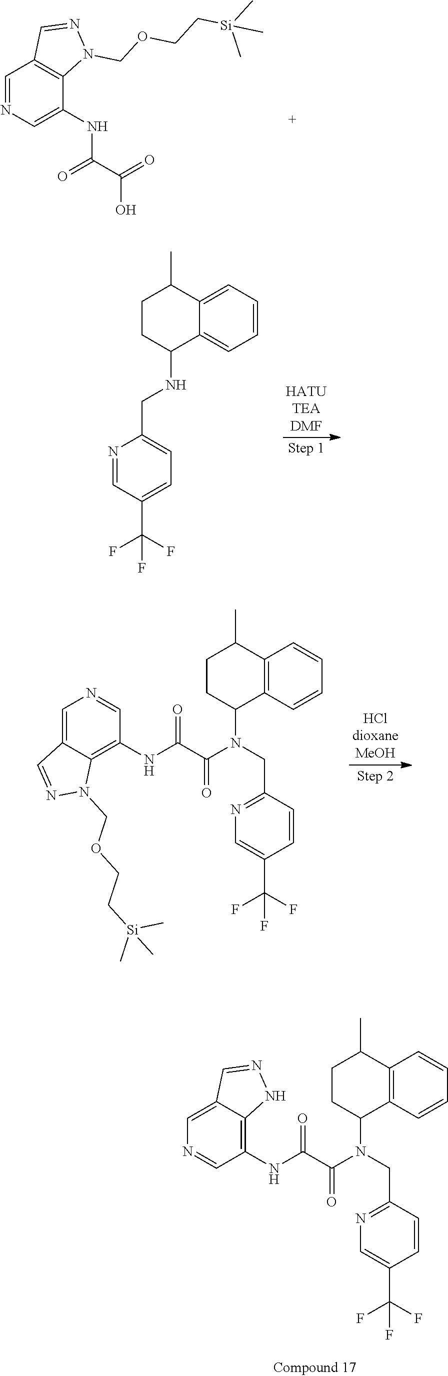 [SMILES: C[Si](C)(C)CCOCn1ncc2cncc(NC(=O)C(=O)O)c21 ; CC1CCC(NCc2ccc(C(F)(F)F)cn2)c2ccccc21 ; CC1CCC(N(Cc2ccc(C(F)(F)F)cn2)C(=O)C(=O)Nc2cncc3cnn(COCC[Si](C)(C)C)c23)c2ccccc21 ; CC1CCC(N(Cc2ccc(C(F)(F)F)cn2)C(=O)C(=O)Nc2cncc3cn[nH]c23)c2ccccc21]

The synthesis of (rac)-4-methyl-N-((5-(trifluoromethyl)pyridin-2-yl)methyl)-1,2,3,4-tetrahydronaphthalen-1-amine is described in Intermediate 4.

### Step 1: N′-(4-methyltetralin-1-yl)-N′-[[5-(trifluoromethyl)-2-pyridyl]methyl]-N-[1-(2-trimethylsilylethoxymethyl)pyrazolo[4,3-c]pyridin-7-yl]oxamide

To the solution of (rac)-4-methyl-N-[5-(trifluoromethyl)-2-pyridyl]methyl]tetralin-1-amine (248.44 mg, 775.51 μmol), 2-oxo-2-[1-(2-trimethylsilylethoxymethyl)pyrazolo[4,3-c]pyridin-7-yl]amino]acetic acid (260.90 mg, 775.51 μmol) and Triethylamine (235.42 mg, 2.33 mmol, 324.27 μL) in DMF (3 mL) HATU (324.36 mg, 853.06 μmol) was added portionwise. Mixture was stirred at 25° C. for 2 hr. The reaction mixture was submitted for reverse phase HPLC (SYSTEM 60-60-100% 0-1-6 min H₂O/MeOH/0.1% FA, flow 30 ml/min (loading pump 4 ml/min ACN) target mass 638 column: Chromatorex 18 SMB 100-ST 100*19 mm 5 μm) to give N′-(4-methyltetralin-1-yl)-N′-[[5-(trifluoromethyl)-2-pyridyl]methyl]-N-[1-(2-trimethylsilylethoxymethyl)pyrazolo[4,3-c]pyridin-7-yl]oxamide (37 mg, 57.93 μmol, 7.47% yield) as a brown solid.

LCMS (ESI): [M+H]⁺m/z: calcd 639.3; found 639.2; Rt=1.587 min.

### Step 2: The synthesis of N′-(4-methyltetralin-1-yl)-N-(1H-pyrazolo[4,3-c]pyridin-7-yl)-N′-[5-(trifluoromethyl)-2-pyridyl]methyl]oxamide

To the stirred solution of N′-(4-methyltetralin-1-yl)-N′-[5-(trifluoromethyl)-2-pyridyl]methyl]-N-[1-(2-trimethylsilylethoxymethyl)pyrazolo[4,3-c]pyridin-7-yl]oxamide (32 mg, 50.10 μmol) in MeOH (0.5 mL) Hydrogen chloride solution 4.0M in dioxane (400.00 mg, 10.97 mmol, 0.5 mL) was added. The resulting mixture was stirred at 25° C. for 14 hr. The reaction mixture was concentrated under reduce pressure. The residue was purified by reverse phase HPLC (SYSTEM 15-65% 0-5 min H₂O/ACN/0.1% FA, flow: 30 ml/min (loading pump 4 ml/min ACN) target mass 508.51 column: Chromatorex 18 SMB100-5T 100×19 mm 5 μm) to give N′-(4-methyltetralin-1-yl)-N-(1H-pyrazolo[4,3-c]pyridin-7-yl)-N′-[[5-(trifluoromethyl)-2-pyridyl]methyl]oxamide (16 mg, 29.36 μmol, 58.61% yield, HCl).

¹H NMR (600 MHz, dmso) δ 1.18-1.25 (m, 3H), 1.34-1.59 (m, 1H), 1.60-1.77 (m, 1H), 1.81-1.99 (m, 2H), 2.01-2.33 (m, 1H), 2.84-2.95 (m, 1H), 3.96-4.64 (m, 1H), 4.64-5.39 (m, 1H), 5.60-5.90 (m, 1H), 7.12-7.19 (m, 1H), 7.19-7.26 (m, 2H), 7.31-7.39 (m, 1H), 7.52-7.67 (m, 1H), 8.01-8.34 (m, 2H), 8.35-8.52 (m, 1H), 8.79-8.99 (m, 2H), 10.88-11.33 (m, 1H), 12.95-13.25 (m, 1H).

LCMS (ESI): [M+H]⁺m/z: calcd 509.2; found 509.2; Rt=3.324 min.

### Example 35. Synthesis of N-(6-amino-5-cyclopropyl-3-pyridyl)-N′-(2-cyclopropylpropyl)-N′-[5-(trifluoromethyl)-2-pyridyl]methyl]oxamide (Compound 13)

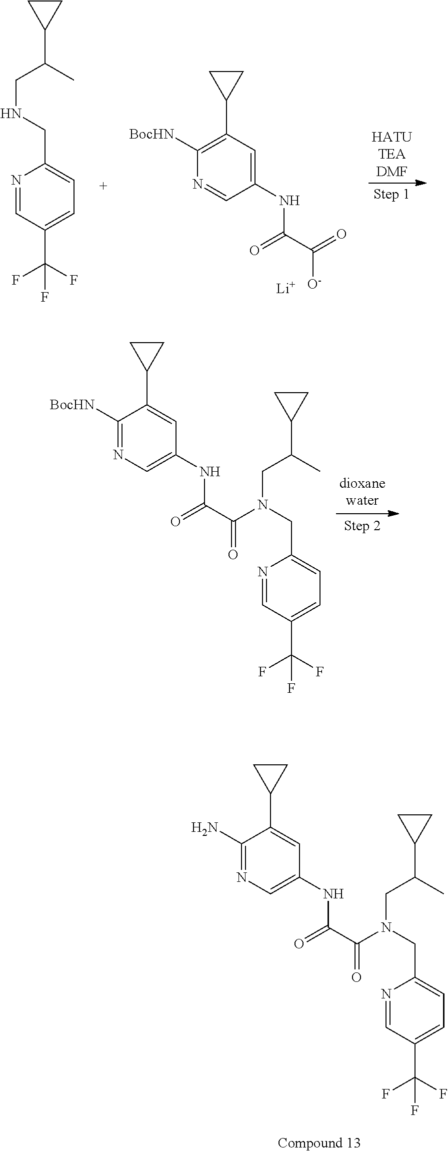 [SMILES: CC(CNCc1ccc(C(F)(F)F)cn1)C1CC1 ; CC(C)(C)OC(=O)Nc1ncc(NC(=O)C(=O)[O-])cc1C1CC1 ; [Li+] ; CC(CN(Cc1ccc(C(F)(F)F)cn1)C(=O)C(=O)Nc1cnc(NC(=O)OC(C)(C)C)c(C2CC2)c1)C1CC1 ; CC(CN(Cc1ccc(C(F)(F)F)cn1)C(=O)C(=O)Nc1cnc(N)c(C2CC2)c1)C1CC1]

The synthesis of 2-cyclopropyl-N-((5-(trifluoromethyl)pyridin-2-yl)methyl)propan-1-amine is described in the following. To a solution of 5-(trifluoromethyl)pyridine-2-carbaldehyde (1.65 g, 9.42 mmol), 2-cyclopropylpropan-1-amine (1.92 g, 14.13 mmol, HCl) in DCE (24.72 mL) was added TEA (1.43 g, 14.13 mmol, 1.97 mL) and the resulting solution was cooled to -10° C. Then, Sodium triacetoxyborohydride (2.00 g, 9.42 mmol) was added and the resulting mixture was left to stir at rt for 12 hr. The reaction mixture was quenched with aq sat NaHCO₃, washed with brine, dried and evaporated to dryness to give 2-cyclopropyl-N-[[5-(trifluoromethyl)-2-pyridyl]methyl]propan-1-amine (2.2 g, 8.52 mmol, 90.40% yield) as a yellow oil. After work-up we realized that in the residue 35% of product and 65% of imine intermediate. This was further reduced with Sodium Borohydride (356.49 mg, 9.42 mmol, 331.93 μL) in MeOH (25 mL). The reaction mixture was evaporated, partitioned in mixture EtOAc/water. Water was extracted with EtOAc (10 ml). Combined organics were dried over Na₂SO₄ and evaporated. 2-cyclopropyl-N-[[5-(trifluoromethyl)-2-pyridyl]methyl]propan-1-amine (2.2 g, 8.52 mmol, 90.40% yield) was obtained as a light-yellow oil.

### Step 1 tert-butyl N-[3-cyclopropyl-5-[[2-[2-cyclopropylpropyl-[[5-(trifluoromethyl)-2-pyridyl]methyl]amino]-2-oxo-acetyl]amino]-2-pyridyl]carbamate

2-cyclopropyl-N-[5-(trifluoromethyl)-2-pyridyl]methyl]propan-1-amine (0.1 g, 387.17 μmol), HATU (147.22 mg, 387.17 μmol) and TEA (117.53 mg, 1.16 mmol, 161.89 μL) were mixed in dry DMF (4.89 mL) at rt and the resulting mixture was stirred for 15 min. lithium 2-[6-(tert-butoxycarbonylamino)-5-cyclopropyl-3-pyridyl]amino]-2-oxo-acetate (126.71 mg, 387.17 μmol) was added thereto and the resulting mixture was stirred at rt overnight. The resulting mixture was poured into water, extracted 3 times with EtOAc, combined organics were washed with water, brine, and evaporated. tert-butyl N-[3-cyclopropyl-5-[2-[2-cyclopropylpropyl-[[5-(trifluoromethyl)-2-pyridyl]methyl]amino]-2-oxo-acetyl]amino]-2-pyridyl]carbamate (155 mg, crude) was used in the next step without further purification.

LCMS (ESI): [M+H]⁺m/z: calcd 562.3; found 562.2; Rt=4.224 min.

### Step 2: The synthesis of N-(6-amino-5-cyclopropyl-3-pyridyl)-N′-(2-cyclopropylpropyl)-N′-[5-(trifluoromethyl)-2-pyridyl]methyl]oxamide

tert-butyl N-[3-cyclopropyl-5-[[2-[2-cyclopropylpropyl-[5-(trifluoromethyl)-2-pyridyl]methyl]amino]-2-oxo-acetyl]amino]-2-pyridyl]carbamate (155 mg, 165.60 μmol) was dissolved in water (2 mL)/dioxane (2 mL) mixture and was heated at 100° C. overnight. The resulting mixture was evaporated to dryness and purified by HPLC (20-50% 0.5-5 min; 30 ml/min water-acetonitrile+NH3 (loading pump 4 ml/min water); column XBridge 19*100 mm (L)) to give N-(6-amino-5-cyclopropyl-3-pyridyl)-N′-(2-cyclopropylpropyl)-N′-[5-(trifluoromethyl)-2-pyridyl]methyl]oxamide (57.7 mg, 125.03 μmol, 75.50% yield) as a beige solid.

¹H NMR (DMSO-d6, 600 MHz): δ (ppm) 0.01-0.56 (m, 7H), 0.84-0.93 (m, 5H), 1.13-1.19 (m, 1H), 1.61-1.67 (m, 1H), 3.22-3.67 (m, 2H), 4.71-4.98 (m, 2H), 5.71-5.76 (m, 2H), 7.23-7.32 (m, 1H), 7.52-7.58 (m, 1H), 7.96-8.04 (m, 1H), 8.18-8.22 (m, 1H), 8.89-8.92 (m, 1H), 10.25-10.37 (m, 1H).

LCMS (ESI): [M+H]⁺m/z: calcd 462.2; found 462.2; Rt=2.448 min.

### Example 36. Synthesis of (R)—N¹-(6-amino-5-cyclopropylpyridin-3-yl)-N2-(1-cyclopropyl-2-methoxyethyl)-N2-((5-(trifluoromethyl)pyridin-2-yl)methyl)oxalamide (Compound 45)

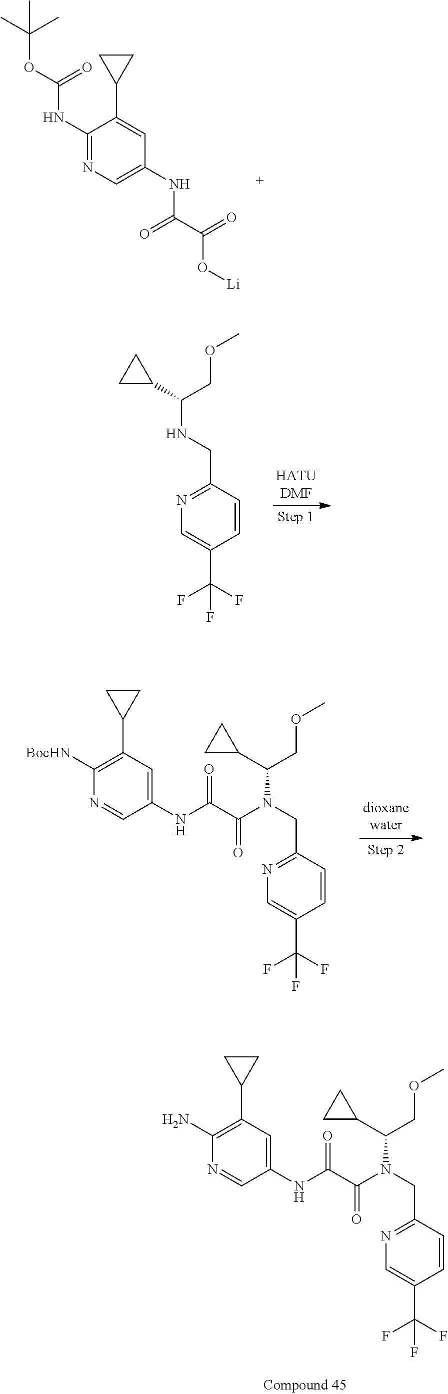

The synthesis of (R)-1-cyclopropyl-2-methoxy-N-((5-(trifluoromethyl)pyridin-2-yl)methyl) ethan-1-amine is described in Intermediate 1.

### Step 1: (R)-tert-butyl(3-cyclopropyl-5-(2-((1-cyclopropyl-2-methoxyethyl)((5-(trifluoromethyl)pyridin-2-yl)methyl)amino)-2-oxoacetamido)pyridin-2-yl)carbamate

To a stirred solution of (1R)-1-cyclopropyl-2-methoxy-N-[5-(trifluoromethyl)-2-pyridyl]methyl]ethanamine (0.1 g, 364.59 μmol) and [2-[6-(tert-butoxycarbonylamino)-5-cyclopropyl-3-pyridyl]amino]-2-oxo-acetyl]oxylithium (0.14 g, 427.79 μmol) in DMF (5 mL) was added HATU (0.15 g, 394.50 μmol) at 5-10° C. The resulting reaction mixture was stirred at 25° C. for 16 hr. Upon completion, the reaction mixture was concentrated under reduced pressure. The desired product (R)-tert-butyl(3-cyclopropyl-5-(2-((1-cyclopropyl-2-methoxyethyl)((5-(trifluoromethyl)pyridin-2-yl)methyl)amino)-2-oxoacetamido)pyridin-2-yl)carbamate (0.21 g, 363.58 μmol, 99.72% yield) was isolated.

LCMS (ESI): [M+H]⁺m/z: calcd 578.3; found 578.2; Rt=1.451 min.

### Step 2: The synthesis of (R)—N¹-(6-amino-5-cyclopropylpyridin-3-yl)-N2-(1-cyclopropyl-2-methoxyethyl)-N2-((5-(trifluoromethyl)pyridin-2-yl)methyl)oxalamide

The solution of tert-butyl N-[3-cyclopropyl-5-[[2-oxo-2-[(1R)-1-cyclopropyl-2-methoxy-ethyl]-[[5-(trifluoromethyl)-2-pyridyl]methyl]amino]acetyl]amino]-2-pyridyl]carbamate (0.21 g, 363.58 μmol) in Dioxane (3 mL) and Water (3 mL) was stirred at 90° C. for 16 hr. Upon completion, the reaction mixture was concentrated under reduced pressure to obtain crude product. The obtained crude product was purified by reverse phase HPLC chromatography (Device (Mobile Phase, Column): SYSTEM 20-20-40% 0-1-5 min H₂O/ACN/0.2% FA, flow: 30 ml/min (loading pump 4 ml/min acetonitrile) target mass 477 column: Chromatorex 18 SMB100-5T 100×19 mm 5 μm) to afford (R)—N¹-(6-amino-5-cyclopropylpyridin-3-yl)-N2-(1-cyclopropyl-2-methoxyethyl)-N2-((5-(trifluoromethyl)pyridin-2-yl)methyl)oxalamide (81 mg, 169.64 μmol, 46.66% yield) as a white solid.

### Example 37. Synthesis of N-(4-amino-1H-pyrazolo[4,3-c]pyridin-7-yl)-N′-benzyl-N′-sec-butyl-oxamide (Compound 48)

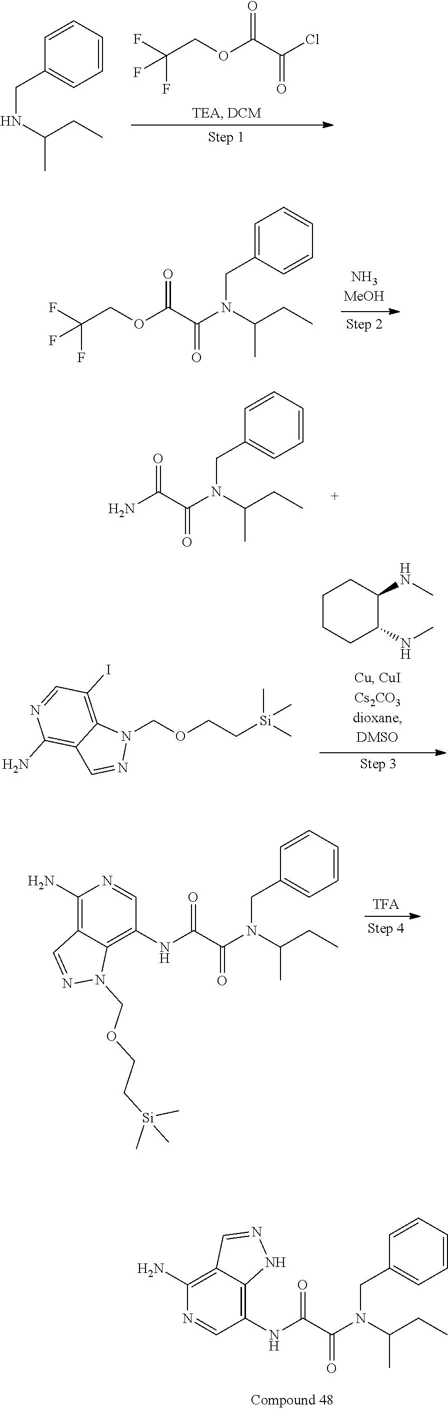

### Step 1:2,2,2-trifluoroethyl 2-[benzyl(sec-butyl)amino]-2-oxo-acetate

2,2,2-trifluoroethyl 2-chloro-2-oxo-acetate (3.19 g, 16.75 mmol) was added dropwise to the cooled to 5° C. solution of N-benzylbutan-2-amine (2.23 g, 11.17 mmol, HCl) in Dichloromethane and Triethylamine (5.65 g, 55.83 mmol, 7.78 mL). After addition was complete, cooling bath was removed and resulting mixture was stirred at 25° C. for 1 hr. Then, volatiles were removed under reduced pressure and residue was partitioned between water (40 ml) and MTBE (50 ml). Organic layer was separated, dried over Na₂SO₄ and concentrated under reduced pressure, leaving 2,2,2-trifluoroethyl 2-[benzyl(sec-butyl)amino]-2-oxo-acetate (3.3 g, 10.40 mmol, 93.14% yield).

LCMS (ESI): [M+H]⁺m/z: calcd 318.1; found 318.0; Rt=1.351 min.

### Step 2: N′-benzyl-N′-sec-butyl-oxamide

2,2,2-trifluoroethyl 2-[benzyl(sec-butyl)amino]-2-oxo-acetate (3.3 g, 10.40 mmol) was dissolved in Ammonia (17 wt. % in MeOH)(15.60 g, 155.72 mmol, 20 mL, 17% purity). Resulting solution was stirred at 25° C. for 16 hr. Then, it was concentrated under reduced pressure, leaving N′-benzyl-N′-sec-butyl-oxamide (2.78 g, crude).

LCMS (ESI): [M+H]⁺m/z: calcd 235.2; found 235.2; Rt=1.146 min.

### Step 3: N-[4-amino-1-(2-trimethylsilylethoxymethyl)pyrazolo[4,3-c]pyridin-7-yl]-N′-benzyl-N′-sec-butyl-oxamide

7-iodo-1-(2-trimethylsilylethoxymethyl)pyrazolo[4,3-c]pyridin-4-amine (310 mg, 794.27 μmol), N′-benzyl-N′-sec-butyl-oxamide (200 mg, 853.63 μmol), Copper (10.10 mg, 158.85 μmol), Copper (I) iodide (45.38 mg, 238.28 μmol, 8.07 μL), (S,S)-(+)—N,N′-Dimethyl-1,2-cyclohexanediamine (33.89 mg, 238.28 μmol, 37.58 μL) and Potassium carbonate (219.55 mg, 1.59 mmol, 95.87 μL) were mixed together in Dioxane (3 mL) and Dimethylsulfoxide (1 mL). Reaction flask was purged with argon and resulting mixture was stirred at 110° C. for 16 hr under inert atmosphere. Then, mixture was filtered and filtrate was subjected to HPLC (60-85% 0-5 min H₂O/MeOH/0.1% NH₄OH, flow: 30 ml/min; column: YMC Triart C18 100×20 mm, 5 μm), affording N-[4-amino-1-(2-trimethylsilylethoxymethyl)pyrazolo[4,3-c]pyridin-7-yl]-N′-benzyl-N′-sec-butyl-oxamide (57 mg, 114.76 μmol, 14.45% yield).

LCMS (ESI): [M+H]⁺m/z: calcd 497.3; found 497.4; Rt=2.878 min.

### Step 4: The synthesis of N-(4-amino-1H-pyrazolo[4,3-c]pyridin-7-yl)-N′-benzyl-N′-sec-butyl-oxamide

N-[4-amino-1-(2-trimethylsilylethoxymethyl)pyrazolo[4,3-c]pyridin-7-yl]-N′-benzyl-N′-sec-butyl-oxamide (57 mg, 114.76 μmol) was dissolved in Trifluoroacetic acid (1.48 g, 12.98 mmol, 1 mL). Resulting solution was stirred at 25° C. for 3 hr. Then, most of TFA was evaporated under reduced pressure and residue was subjected to HPLC (20-70% 0-5 min H₂O/MeOH/0.1% NH₄OH, flow: 30 ml/min; column: XBridge C18 100×19 mm, 5 μm), affording N-(4-amino-1H-pyrazolo[4,3-c]pyridin-7-yl)-N′-benzyl-N′-sec-butyl-oxamide (6 mg, 16.37 μmol, 14.27% yield).

¹H NMR (600 MHz, dmso) δ 0.60-0.79 (m, 3H), 0.99-1.17 (m, 3H), 1.39-1.56 (m, 1H), 1.56-1.71 (m, 1H), 3.68-4.47 (m, 2H), 4.59-4.78 (m, 1H), 6.42-6.81 (m, 2H), 6.92-7.25 (m, 2H), 7.26-7.31 (m, 1H), 7.32-7.43 (m, 2H), 7.48-7.75 (m, 1H), 8.06-8.23 (m, 1H), 9.49-10.54 (m, 1H), 12.55-13.36 (m, 1H).

LCMS (ESI): [M+H]⁺m/z: calcd 367.2; found 367.0; Rt=0.838 min.

### Example 38. Synthesis of N-(6-amino-5-methyl-3-pyridyl)-N′—[(1S)-1-pyrimidin-2-ylethyl]-N′-[5-(trifluoromethyl)-2-pyridyl]methyl]oxamide (Compound 49) and N-(6-amino-5-methyl-3-pyridyl)-N′—[(1R)-1-pyrimidin-2-ylethyl]-N′-[[5-(trifluoromethyl)-2-pyridyl]methyl]oxamide (Compound 6)

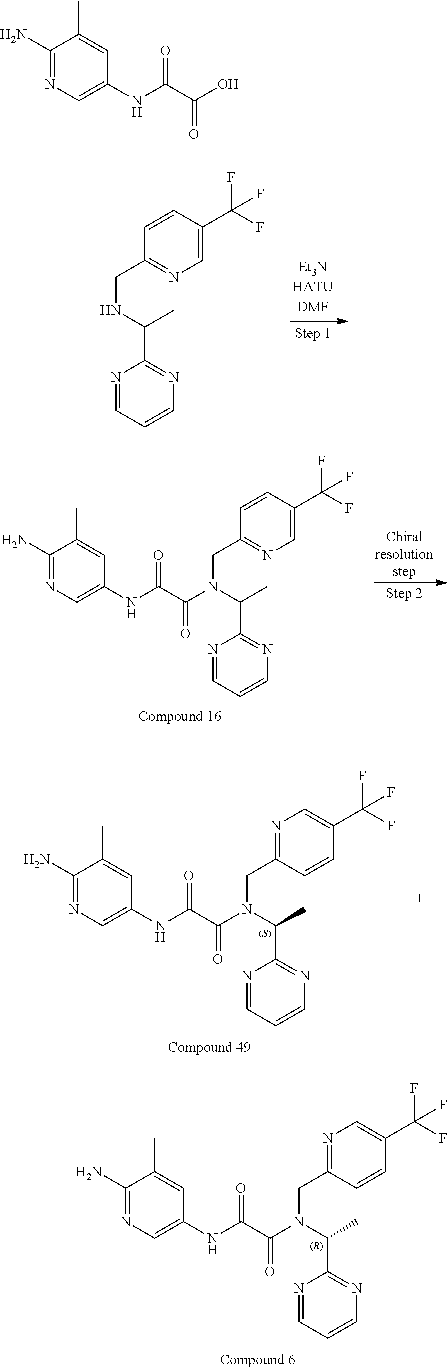

### Step 1: N-(6-amino-5-methyl-3-pyridyl)-N′-(1-pyrimidin-2-ylethyl)-N′-115-(trifluoromethyl)-2-pyridyl]methyl]oxamide (Compound 16)

HATU (232.37 mg, 611.13 μmol) was added in small portions over 0.5 hr period to a stirred mixture of 1-pyrimidin-2-yl-N-[[5-(trifluoromethyl)-2-pyridyl]methyl]ethanamine (150 mg, 531.42 μmol), 2-[(6-amino-5-methyl-3-pyridyl)amino]-2-oxo-acetic acid (124.46 mg, 637.70 μmol) and triethyl amine (322.64 mg, 3.19 mmol, 444.41 μL) in DMF (2.5 mL) at 25° C. The resulting mixture was stirred at 25° C. for 12 hr, and then submitted to reverse phase HPLC (column: XBridge BEH C18 100×19 mm, 5 μm; mobile phase: 20-70% 0-5 min H₂O/MeOH/0.1% NH₄OH; flow rate: 30 ml/min (loading pump 4 ml/min methanol)) to afford Compound 16 N-(6-amino-5-methyl-3-pyridyl)-N′-(1-pyrimidin-2-ylethyl)-N′-[[5-(trifluoromethyl)-2-pyridyl]methyl]oxamide (49 mg, 106.66 μmol, 20.07% yield) as a light-brown solid.

LCMS (ESI): [M+H]⁺m/z: calcd 460.2; found 460.2; Rt=1.977 min.

### Step 2: The synthesis of N-(6-amino-5-methyl-3-pyridyl)-N′—[(1R)-1-pyrimidin-2-ylethyl]-N′-[[5-(trifluoromethyl)-2-pyridyl]methyl]oxamide (Compound 6) and N-(6-amino-5-methyl-3-pyridyl)-N′—[(1S)-1-pyrimidin-2-yl]ethyl]-N′-[[5-(trifluoromethyl)-2-pyridyl]methyl]oxamide (Compound 49)

A racemic N-(6-amino-5-methyl-3-pyridyl)-N′-(1-pyrimidin-2-ylethyl)-N′-[[5-(trifluoromethyl)-2-pyridyl]methyl]oxamide (30 mg, 65.30 μmol) was submitted to preparative chiral HPLC (Chiralpak IB (250×20 mm, 5 mkm); Mobile Phase: CO₂: MeOH, 85:15; Flow Rate: 50.0 ml/min) to afford:

N-(6-amino-5-methyl-3-pyridyl)-N′—[(1R)-1-pyrimidin-2-ylethyl]-N′-[[5-(trifluoromethyl)-2-pyridyl]methyl]oxamide (7.9 mg, 17.20 μmol, 26.33% yield)(RT=6.57 min, Compound 6) as a yellow solid;

¹H NMR (DMSO-d6, 600 MHz): δ (ppm) 1.50-1.52 (m, 3H), 1.95-2.02 (m, 3H), 4.56-5.17 (m, 2H), 5.57-5.72 (m, 3H), 7.31-7.51 (m, 2H), 7.65-8.12 (m, 3H), 8.72-8.80 (m, 3H), 10.32-10.49 (m, 1H).

LCMS (ESI): [M+1]⁺m/z: calcd 460.2; found 460.0; Rt=1.914 min. and N-(6-amino-5-methyl-3-pyridyl)-N′—[(1S)-1-pyrimidin-2-ylethyl]-N′-[5-(trifluoromethyl)-2-pyridyl]methyl]oxamide (7.5 mg, 16.32 μmol, 25.00% yield)(RT=7.86 min, Compound 49) as a yellow solid.

The absolute stereochemistry of the two compounds was independently confirmed.

¹H NMR (DMSO-d6, 600 MHz): δ (ppm) 1.50-1.63 (m, 3H), 1.95-2.02 (m, 3H), 4.56-4.88 (m, 1H), 4.95-5.17 (m, 1H), 5.57-5.72 (m, 3H), 7.31-7.66 (m, 3H), 7.86-8.11 (m, 2H), 8.72-8.80 (m, 3H), 10.32-10.49 (m, 1H).

LCMS (ESI): [M+1]⁺m/z: calcd 460.2; found 460.0; Rt=1.914 min.

### Example 39. Synthesis of (R)—N¹-(6-amino-5-ethylpyridin-3-yl)-N2-(1-cyclopropyl-2-methoxyethyl)-N2-((5-(trifluoromethyl)pyridin-2-yl)methyl)oxalamide (Compound 7)

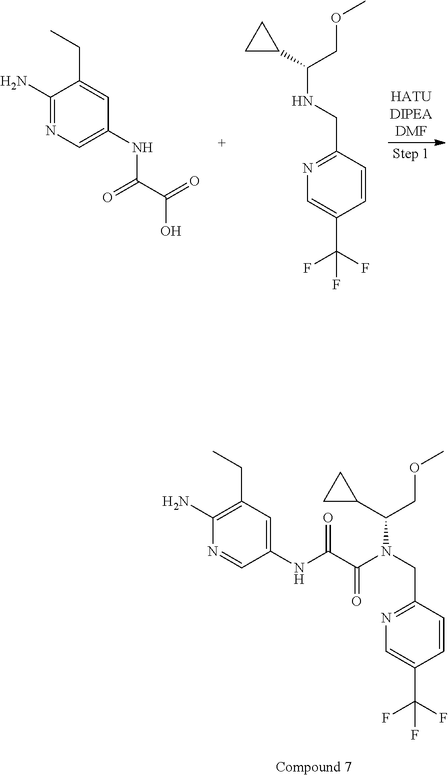 [SMILES: CCc1cc(NC(=O)C(=O)O)cnc1N ; COC[C@H](NCc1ccc(C(F)(F)F)cn1)C1CC1 ; CCc1cc(NC(=O)C(=O)N(Cc2ccc(C(F)(F)F)cn2)[C@@H](COC)C2CC2)cnc1N]

The synthesis of (R)-1-cyclopropyl-2-methoxy-N-((5-(trifluoromethyl)pyridin-2-yl)methyl) ethan-1-amine is described in Intermediate 1.

### Step 1. The synthesis of (R)—N¹-(6-amino-5-ethylpyridin-3-yl)-N2-(1-cyclopropyl-2-methoxyethyl)-N2-((5-(trifluoromethyl)pyridin-2-yl)methyl)oxalamide

To a stirred solution of (1R)-1-cyclopropyl-2-methoxy-N-[[5-(trifluoromethyl)-2-pyridyl]methyl]ethanamine (0.1 g, 364.59 μmol), 2-[(6-amino-5-ethyl-3-pyridyl)amino]-2-oxo-acetic acid (0.09 g, 430.21 μmol) and N,N-Diisopropylethylamine (148.40 mg, 1.15 mmol, 0.2 mL) in DMF (5 mL) was added HATU (0.15 g, 394.50 μmol) at 5-10° C. The resulting reaction mixture was stirred at 25° C. for 16 hr. Upon completion, the reaction mixture was concentrated under reduced pressure to obtain crude product. The obtained crude product was purified by reverse phase HPLC chromatography (Device (Mobile Phase, Column): SYSTEM 20-20-40% 0-1-5 min H₂O/ACN/0.2% FA, flow: 30 ml/min (loading pump 4 ml/min acetonitrile) target mass 465 column: Chromatorex 18 SMB100-5T 100×19 mm 5 μm) to afford (R)—N¹-(6-amino-5-ethylpyridin-3-yl)-N2-(1-cyclopropyl-2-methoxyethyl)-N2-((5-(trifluoromethyl)pyridin-2-yl)methyl)oxalamide (59 mg, 126.75 μmol, 34.77% yield) as a white solid.

¹H NMR (DMSO-d6, 600 MHz): δ (ppm) 0.13-0.53 (m, 4H), 0.95-1.11 (m, 4H), 2.30-2.40 (m, 2H), 3.09-3.13 (m, 3H), 3.43-3.80 (m, 3H), 4.75-5.07 (m, 2H), 5.58-5.63 (m, 2H), 7.28-7.47 (m, 1H), 7.57-7.78 (m, 1H), 7.88-8.04 (m, 1H), 8.15-8.20 (m, 1H), 8.82-8.88 (m, 1H), 10.28-10.38 (m, 1H).

LCMS (ESI): [M+H]⁺m/z: calcd 466.2; found 466.2; Rt=2.339 min.

### Example 40. Synthesis of N1-((1R,2R)-2-(difluoromethoxy)cyclopentyl)-N2-(imidazo[1,2-a]pyridin-7-yl)-N1-((5-(trifluoromethyl)pyridin-2-yl)methyl)oxalamide (Compound 41)

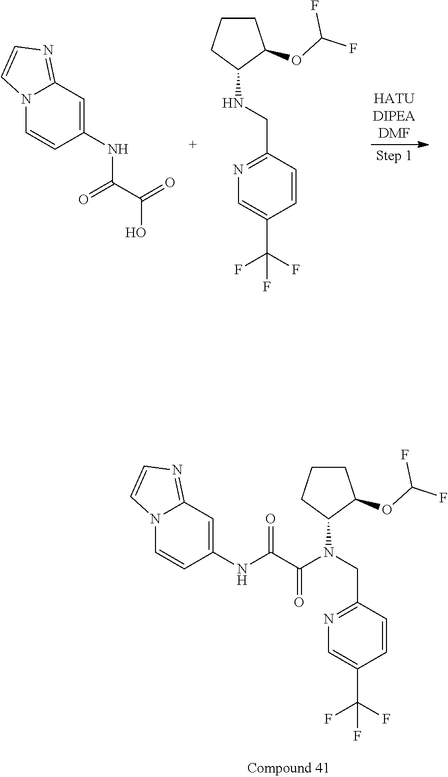 [SMILES: O=C(O)C(=O)Nc1ccn2ccnc2c1 ; FC(F)O[C@@H]1CCC[C@H]1NCc1ccc(C(F)(F)F)cn1 ; O=C(Nc1ccn2ccnc2c1)C(=O)N(Cc1ccc(C(F)(F)F)cn1)[C@@H]1CCC[C@H]1OC(F)F]

The synthesis of (1R,2R)-2-(difluoromethoxy)-N-((5-(trifluoromethyl)pyridin-2-yl)methyl)cyclopentan-1-amine is described in Intermediate 3.

### Step 1. The synthesis of rac N1-((1R,2R)-2-(difluoromethoxy)cyclopentyl)-N2-(imidazo[1,2-a]pyridin-7-yl)-N1-((5-(trifluoromethyl)pyridin-2-yl)methyl)oxalamide

DIPEA (154.33 mg, 1.19 mmol, 208.00 μL) was added to the solution of respective 2-(imidazo[1,2-a]pyridin-7-ylamino)-2-oxo-acetic acid (0.07 g, 341.18 μmol) and (1R,2R)-2-(difluoromethoxy)-N-[[5-(trifluoromethyl)-2-pyridyl]methyl]cyclopentanamine (105.86 mg, 341.18 μmol) in DMF (9.79 mL). The resulting mixture was stirred for 5 min followed by the addition of HATU (142.70 mg, 375.30 μmol). Then, the reaction mixture was stirred overnight at rt. After the completion of the reaction, monitored by LCMS, the resulting suspension was concentrated under reduced pressure. It was dissolved in EtOAc (20 ml) and washed with water (5 ml), brine (5 ml), dried over Na₂SO₄ and evaporated. The obtained solid was subjected to HPLC (Waters Sunfire C18 20*100 5 mkm column and MeOH+NH3 as an eluent mixture) to afford rac N1-((1R,2R)-2-(difluoromethoxy)cyclopentyl)-N2-(imidazo[1,2-a]pyridin-7-yl)-N1-((5-(trifluoromethyl)pyridin-2-yl)methyl)oxalamide (15.4 mg, 30.96 μmol, 9.07% yield) as a yellow solid.

¹H NMR (DMSO-d6, 600 MHz): δ (ppm) 1.65 (m, 4H), 1.95 (m, 2H), 4.48 (m, 1H), 4.75 (m, 2H), 5.01 (m, 1H), 6.59 (m, 1H), 7.08 (m, 1H), 7.55 (m, 2H), 7.88 (m, 2H), 8.17 (m, 1H), 8.45 (m, 1H), 8.88 (m, 1H), 10.96 (m, 1H).

LCMS (ESI): [M+H]⁺m/z: calcd 498.2; found 498.2; Rt=2.922 min.

### Example 41. Synthesis of N-(4-amino-1H-pyrazolo[4,3-c]pyridin-7-yl)-N′,N′-bis[(3,5-difluorophenyl)methyl]oxamide (Compound 2)

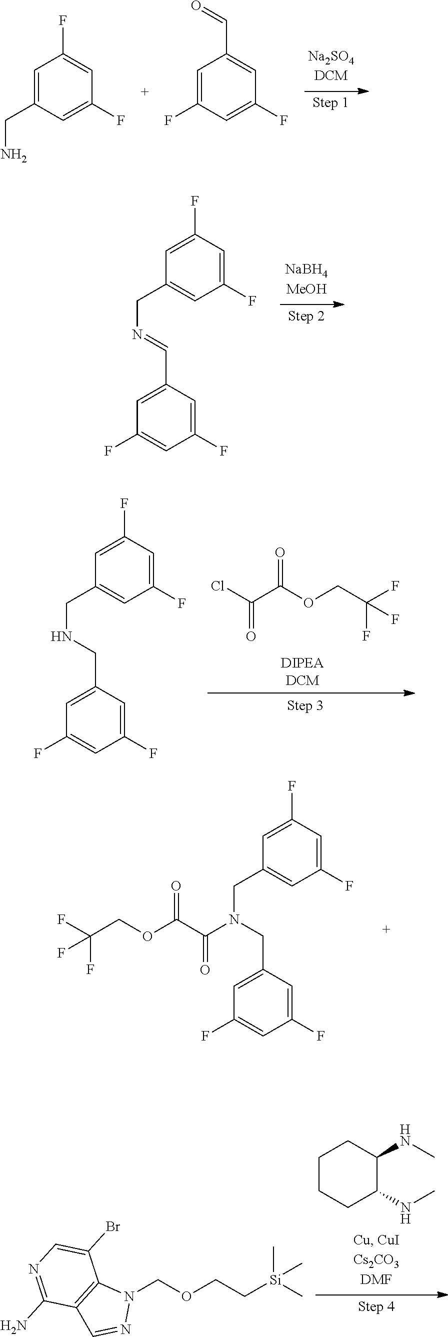

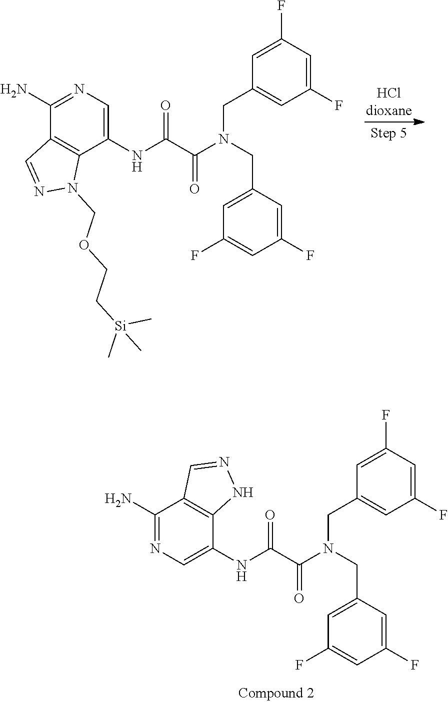 [SMILES: C[Si](C)(C)CCOCn1ncc2c(N)ncc(NC(=O)C(=O)N(Cc3cc(F)cc(F)c3)Cc3cc(F)cc(F)c3)c21 ; Nc1ncc(NC(=O)C(=O)N(Cc2cc(F)cc(F)c2)Cc2cc(F)cc(F)c2)c2[nH]ncc12]

### Step 1: (E)-1-(3,5-difluorophenyl)-N-[(3,5-difluorophenyl)methyl]methanimine

(3,5-difluorophenyl)methanamine (4.5 g, 31.44 mmol, 3.72 mL), 3,5-difluorobenzaldehyde (4.47 g, 31.44 mmol) and Sodium sulfate, anhydrous (10 g, 70.40 mmol, 3.73 mL) was mixed in DCM (80 mL) and stirred at RT overnight. Upon completion, the reaction mixture was faltered and filtrate was concentrated under reduced pressure to afford (E)-1-(3,5-difluorophenyl)-N-[(3,5-difluorophenyl)methyl]methanimine (7 g, crude) which was directly used in the next step without purification.

### Step 2:1-(3,5-difluorophenyl)-N-[(3,5-difluorophenyl)methyl]methanamine

(E)-1-(3,5-difluorophenyl)-N-[(3,5-difluorophenyl)methyl]methanimine (7 g, 26.20 mmol) was dissolved in methanol (100 mL) followed by portionwise addition of Sodium Borohydride (1.09 g, 28.82 mmol, 1.02 mL). The reaction mixture was stirred at RT overnight and concentrated under reduced pressure. The residue was partitioned between DCM and water. Organic layer was separated, dried over Na₂SO₄ and concentrated on rotary evaporator to afford 1-(3,5-difluorophenyl)-N-[(3,5-difluorophenyl)methyl]methanamine (7.9 g, crude).

LCMS (ESI): [M+H]⁺m/z: calcd 270.1; found 270.0; Rt=0.765 min.

### Step 3:2,2,2-trifluoroethyl 2-[bis[(3,5-difluorophenyl)methyl]amino]-2-oxo-acetate

2,2,2-trifluoroethyl 2-chloro-2-oxo-acetate (2.97 g, 15.60 mmol) was added dropwise to an ice bath cooled stirred solution of 1-(3,5-difluorophenyl)-N-[(3,5-difluorophenyl)methyl]methanamine (4 g, 14.86 mmol) and DIPEA (2.50 g, 19.31 mmol, 3.36 mL) in DCM (50 mL). The reaction mixture was stirred overnight and concentrated on rotary evaporator. Upon completion, the reaction mixture was washed with water. The organic layer was separated, dried over Na₂SO₄ and concentrated under reduced pressure to afford 2,2,2-trifluoroethyl 2-[bis[(3,5-difluorophenyl)methyl]amino]-2-oxo-acetate (5 g, crude) which was used in the next step without purification.

LCMS (ESI): [M+H]⁺m/z: calcd 424.1; found 423.9; Rt=1.464 min.

### Step 4: N-[4-amino-1-(2-trimethylsilylethoxymethyl)pyrazolo[4,3-c]pyridin-7-yl]-N′,N′-bis[(3,5-difluorophenyl)methyl]oxamide

N′,N′-bis[(3,5-difluorophenyl)methyl]oxamide (0.3 g, 881.65 μmol), 7-bromo-1-(2-trimethylsilylethoxymethyl)pyrazolo[4,3-c]pyridin-4-amine (302.66 mg, 881.65 μmol), copper (0.005 g, 78.68 μmol), Copper (I) iodide (167.91 mg, 881.65 μmol, 29.88 μL), (1S,2S)-N,N′-Bis -methyl-1,2-cyclohexane-diamine (125.41 mg, 881.65 μmol, 139.03 μL) were mixed in dioxane (10 mL). The reaction mixture was stirred under Ar atmosphere at 100° C. for 48 hr. Upon completion, the mixture concentrated under reduced pressure, treated with DMSO and filtered. The filtrate was submitted to HPLC to afford N-[4-amino-1-(2-trimethylsilylethoxymethyl)pyrazolo[4,3-c]pyridin-7-yl]-N′,N′-bis[(3,5-difluorophenyl)methyl]oxamide (0.085 g, 141.04 μmol, 16.00% yield).

LCMS (ESI): [M+H]⁺m/z: calcd 603.3; found 603.0; Rt=1.399 min.

### Step 5: The synthesis of N-(4-amino-1H-pyrazolo[4,3-c]pyridin-7-yl)-N′,N′-bis[(3,5-difluorophenyl)methyl]oxamide

N-[4-amino-1-(2-trimethylsilylethoxymethyl)pyrazolo[4,3-c]pyridin-7-yl]-N′,N′-bis[(3,5-difluorophenyl)methyl]oxamide (0.085 g, 141.04 μmol) was dissolved in HCl/dioxane solution (12%, 2 ml) and stirred at 25° C. for 2 hr. Upon completion, reaction mixture was concentrated under reduced pressure and residue was submitted to reverse phase HPLC to afford N-(4-amino-1H-pyrazolo[4,3-c]pyridin-7-yl)-N′,N′-bis[(3,5-difluorophenyl)methyl]oxamide (0.012 g, 25.40 μmol, 18.01% yield).

HPLC: 2-10 min 40-70% methanol flow 30 ml.

¹H NMR (600 MHz, dmso) δ 4.52-4.58 (m, 2H), 4.63-4.92 (m, 2H), 6.96-7.10 (m, 3H), 7.10-7.22 (m, 4H), 7.72-8.15 (m, 1H), 8.48-9.04 (m, 2H), 10.80-13.16 (m, 1H), 13.44-15.08 (m, 1H).

LCMS (ESI): [M+H]⁺m/z: calcd 473.2; found 473.0; Rt=1.499 min.

### Example 42. Synthesis of(S)-N1-(6-amino-5-methylpyridin-3-yl)-N2-(1-cyclopropyl-2-methoxyethyl)-N2-((5-(trifluoromethyl)pyridin-2-yl)methyl)oxalamide (Compound 24)

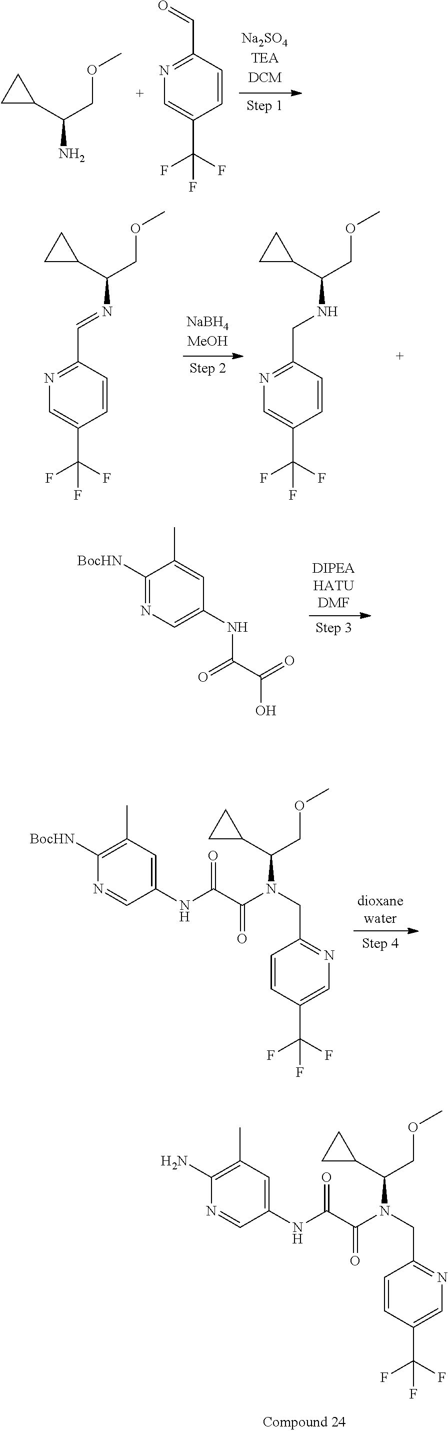

### Step 1: (S,E)-1-cyclopropyl-2-methoxy-N-((5-(trifluoromethyl)pyridin-2-yl)methylene)ethanamine

To a stirred solution of (1S)-1-cyclopropyl-2-methoxy-ethanamine (0.4 g, 2.64 mmol, HCl) and 5-(trifluoromethyl)pyridine-2-carbaldehyde (0.55 g, 3.14 mmol) in CH₂Cl₂ (10 mL) were added Triethylamine (290.40 mg, 2.87 mmol, 400.00 μL) and Sodium sulfate, anhydrous (2.00 g, 14.08 mmol, 746.27 μL). The resulting reaction mixture was stirred at 25° C. for 16 hr. Upon completion, the reaction mixture was evaporated, quenched with water (20 mL). The aqueous phase was extracted with CHCl₃ (2×20 mL). The combined organic phase was dried over Na₂SO₄ and concentrated under reduced pressure. The desired product (S,E)-1-cyclopropyl-2-methoxy-N-((5-(trifluoromethyl)pyridin-2-yl)methylene)ethanamine (0.7 g, 2.57 mmol, 97.46% yield) was isolated.

### Step 2: (S)-1-cyclopropyl-2-methoxy-N-((5-(trifluoromethyl)pyridin-2-yl)methyl)ethanamine

To a stirred solution of (E)-N-[(1S)-1-cyclopropyl-2-methoxy-ethyl]-1-[5-(trifluoromethyl)-2-pyridyl]methanimine (0.7 g, 2.57 mmol) in MeOH (15 mL) was added Sodium Borohydride (0.3 g, 7.93 mmol, 279.33 μL). The resulting reaction mixture was stirred at 25° C. for 16 hr. Upon completion, the reaction mixture was evaporated, quenched with water (20 mL). The aqueous phase was extracted with CHCl₃ (2×20 mL). The combined organic phase was dried over Na₂SO₄ and concentrated under reduced pressure. The desired product(S)-1-cyclopropyl-2-methoxy-N-((5-(trifluoromethyl)pyridin-2-yl)methyl)ethanamine (0.7 g, 2.55 mmol, 99.27% yield) was isolated.

### Step 3: (S)-tert-butyl(5-(2-((1-cyclopropyl-2-methoxyethyl)((5-(trifluoromethyl)pyridin-2-yl)methyl)amino)-2-oxoacetamido)-3-methylpyridin-2-yl)carbamate

To a stirred solution of (1S)-1-cyclopropyl-2-methoxy-N-[[5-(trifluoromethyl)-2-pyridyl]methyl]ethanamine (0.1 g, 364.59 μmol), 2-[[6-(tert-butoxycarbonylamino)-5-methyl-3-pyridyl]amino]-2-oxo-acetic acid (0.12 g, 406.38 μmol) and N,N-Diisopropylethylamine (148.40 mg, 1.15 mmol, 0.2 mL) in DMF (5 mL) was added HATU (0.15 g, 394.50 μmol) at 5-10° C. The resulting reaction mixture was stirred at 25° C. for 16 hr. Upon completion, the reaction mixture was concentrated under reduced pressure to obtain crude product. The desired product(S)-tert-butyl(5-(2-((1-cyclopropyl-2-methoxyethyl)((5-(trifluoromethyl)pyridin-2-yl)methyl)amino)-2-oxoacetamido)-3-methylpyridin-2-yl)carbamate (0.2 g, 362.61 μmol, 99.46% yield) was isolated.

LCMS (ESI): [M+H]⁺m/z: calcd 552.3; found 552.2; Rt=1.460 min.

### Step 4: The synthesis of(S)-N1-(6-amino-5-methylpyridin-3-yl)-N2-(1-cyclopropyl-2-methoxyethyl)-N2-((5-(trifluoromethyl)pyridin-2-yl)methyl)oxalamide

The solution of tert-butyl N-[3-methyl-5-[2-oxo-2-[[(1S)-1-cyclopropyl-2-methoxy-ethyl]-[5-(trifluoromethyl)-2-pyridyl]methyl]amino]acetyl]amino]-2-pyridyl]carbamate (0.2 g, 362.61 μmol) in Dioxane (3 mL) and Water (3 mL) was stirred at 90° C. for 16 hr. Upon completion, the reaction mixture was concentrated under reduced pressure to obtain crude product. The obtained crude product was purified by reverse phase HPLC chromatography (Device (Mobile Phase, Column): SYSTEM 30-80% 0-5 min H₂O/MeOH/0.1% NH₄OH, flow: 30 ml/min (loading pump 4 ml/min methanol) target mass 451.45 column: XBridge C18 100×19 mm, 5 μm) to afford(S)-N1-(6-amino-5-methylpyridin-3-yl)-N2-(1-cyclopropyl-2-methoxyethyl)-N2-((5-(trifluoromethyl)pyridin-2-yl)methyl)oxalamide (86 mg, 190.50 μmol, 52.54% yield) as a white solid.

¹H NMR (DMSO-d6, 600 MHz): δ (ppm) 0.14-0.51 (m, 4H), 0.94-0.96 (m, 1H), 1.95-2.01 (m, 3H), 3.09-3.13 (m, 3H), 3.43-3.81 (m, 3H), 4.74-4.85 (m, 1H), 5.00-5.07 (m, 1H), 5.56-5.61 (m, 2H), 7.28-8.01 (m, 3H), 8.15-8.20 (m, 1H), 8.82-8.88 (m, 1H), 10.28-10.38 (m, 1H).

LCMS (ESI): [M+H]⁺m/z: calcd 452.2; found 452.0; Rt=2.289 min.

### Example 43. Synthesis of (R)—N¹-(2,3-dihydro-1H-inden-1-yl)-N2-(imidazo[1,2-a]pyridin-7-yl)-N1-((5-(trifluoromethyl)pyridin-2-yl)methyl)oxalamide (Compound 15)

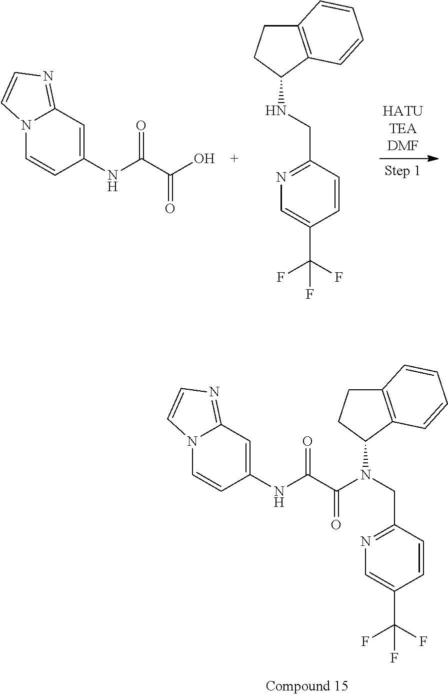 [SMILES: O=C(O)C(=O)Nc1ccn2ccnc2c1 ; FC(F)(F)c1ccc(CN[C@@H]2CCc3ccccc32)nc1 ; O=C(Nc1ccn2ccnc2c1)C(=O)N(Cc1ccc(C(F)(F)F)cn1)[C@@H]1CCc2ccccc21]

The synthesis of (R)—N-((5-(trifluoromethyl)pyridin-2-yl)methyl)-2,3-dihydro-1H-inden-1-amine is above is described in Intermediate 2.

### Step 1. The synthesis of (R)—N¹-(2,3-dihydro-1H-inden-1-yl)-N2-(imidazo[1,2-a]pyridin-7-yl)-N1-((5-(trifluoromethyl)pyridin-2-yl)methyl)oxalamide

HATU (214.64 mg, 564.49 μmol) was added portionwise at r.t. to a suspension of 2-(imidazo[1,2-a]pyridin-7-ylamino)-2-oxo-acetic acid (115.82 mg, 564.49 μmol), (1R)—N-[[5-(trifluoromethyl)-2-pyridyl]methyl]indan-1-amine (150 mg, 513.17 μmol) and TEA (311.57 mg, 3.08 mmol, 429.16 μL) in DMF (6 mL). The clear solution was stirred at 20° C. for 18 hr and the solvents were evaporated in vacuo. The residue was purified by RP HPLC (column: XBridge BEH C18 100×19 mm, 5 μm; mobile phase: 25-50% 0-5 min H₂O/Acetonitrile/0.1% NH₄OH, flow: 30 ml/min (loading pump 4 ml/min acetonitrile)) to give (R)—N¹-(2,3-dihydro-1H-inden-1-yl)-N2-(imidazo[1,2-a]pyridin-7-yl)-N1-((5-(trifluoromethyl)pyridin-2-yl)methyl)oxalamide (52 mg, 108.46 μmol, 21.13% yield) as a beige solid.

¹H NMR (DMSO-d6, 600 MHz): δ (ppm) 2.00 (m, 1H), 2.85 (m, 3H), 4.59 (m, 2H), 5.89 (dt, 1H), 7.17 (m, 4H), 7.38 (m, 1H), 7.50 (m, 2H), 7.84 (d, 1H), 8.02 (m, 1H), 8.18 (m, 1H), 8.44 (m, 1H), 8.79 (m, 1H), 11.09 (m, 1H).

LCMS (ESI): [M+H]⁺m/z: calcd 480.2; found 480.0; Rt=2.444 min.

### Example 44. Synthesis of N-(4-amino-1H-pyrazolo[4,3-c]pyridin-7-yl)-N′-(1,2-dimethylpropyl)-N′-[[5-(trifluoromethyl)-2-pyridyl]methyl]oxamide (Compound 46)

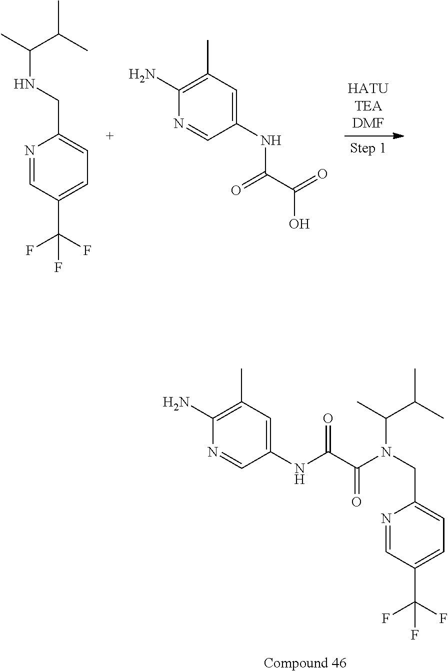 [SMILES: CC(C)C(C)NCc1ccc(C(F)(F)F)cn1 ; Cc1cc(NC(=O)C(=O)O)cnc1N ; Cc1cc(NC(=O)C(=O)N(Cc2ccc(C(F)(F)F)cn2)C(C)C(C)C)cnc1N]

The synthesis of 3-methyl-N-((5-(trifluoromethyl)pyridin-2-yl)methyl) butan-2-amine is described in Intermediate 5.

### Step 1. The synthesis of N-(6-amino-5-methyl-3-pyridyl)-N′-(1,2-dimethylpropyl)-N′-[[5-(trifluoromethyl)-2-pyridyl]methyl]oxamide

To a solution of 3-methyl-N-[[5-(trifluoromethyl)-2-pyridyl]methyl]butan-2-amine (190 mg, 771.51 μmol), 2-[(6-amino-5-methyl-3-pyridyl)amino]-2-oxo-acetic acid (180.69 mg, 925.81 μmol) and Triethylamine (390.34 mg, 3.86 mmol, 537.66 μL) in DMF (4 mL), HATU (396.02 mg, 1.04 mmol) was added. The resulting mixture was stirred at 40° C. for 4 hr. The reaction mixture was purified by HPLC (Device (Mobile Phase, Column): SYSTEM 30-80% 0-5 min H₂O/MeOH/0.1% NH₄OH, flow: 30 ml/min (loading pump 4 ml/min methanol) target mass 423.44 column: XBridge C18 100×19 mm, 5 μm) to give N-(6-amino-5-methyl-3-pyridyl)-N′-(1,2-dimethylpropyl)-N′-[5-(trifluoromethyl)-2-pyridyl]methyl]oxamide (132 mg, 311.74 μmol, 40.41% yield) as a light-yellow solid.

¹H NMR (600 MHz, dmso) 8 0.81-0.91 (m, 6H), 1.03-1.21 (m, 3H), 1.82-1.91 (m, 1H), 1.93-2.06 (m, 3H), 3.65-4.13 (m, 1H), 4.45-5.08 (m, 2H), 5.51-5.69 (m, 2H), 7.17-7.51 (m, 1H), 7.52-7.69 (m, 1H), 7.79-8.04 (m, 1H), 8.11-8.23 (m, 1H), 8.81-8.92 (m, 1H), 10.10-10.49 (m, 1H).

LCMS (ESI): [M+H]⁺m/z: calcd 424.2; found 424.2; Rt=2.325 min.

### Example 45. Synthesis of N-(5-carbamoyl-3-pyridyl)-N′-(1,2-dimethylpropyl)-N′-[5-(trifluoromethyl)-2-pyridyl]methyl]oxamide (Compound 20)

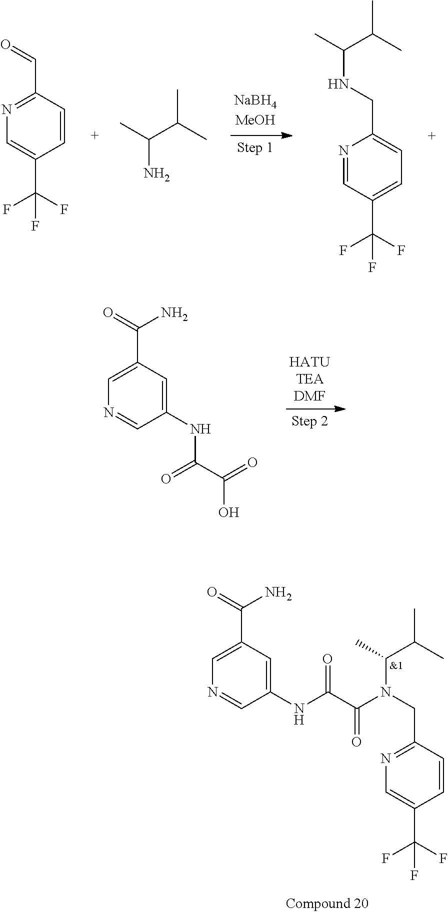

The synthesis of 3-methyl-N-((5-(trifluoromethyl)pyridin-2-yl)methyl) butan-2-amine is described in Intermediate 5.

### Step 1: The synthesis of N-(5-carbamoyl-3-pyridyl)-N′-(1,2-dimethylpropyl)-N′-[[5-(trifluoromethyl)-2-pyridyl]methyl]oxamide

To a solution of 3-methyl-N-[5-(trifluoromethyl)-2-pyridyl]methyl]butan-2-amine (190 mg, 771.51 μmol), 2-[(5-carbamoyl-3-pyridyl)amino]-2-oxo-acetic acid (227.40 mg, 925.81 μmol, HCl) and Triethylamine (390.34 mg, 3.86 mmol, 537.66 μL) in DMF (4 mL), HATU (396.02 mg, 1.04 mmol) was added. The resulting mixture was stirred at 40° C. for 4 hr. The reaction mixture was purified by HPLC (Device (Mobile Phase, Column): SYSTEM 15-15-65% 0-1-6 min H₂O/MeOH/0.1% NH₄OH, flow: 30 ml/min (loading pump 4 ml/min acetonitrile) target mass 437.43 column: XBridge C18 100×19 mm, 5 μm) to give N-(5-carbamoyl-3-pyridyl)-N′-(1,2-dimethylpropyl)-N′-[[5-(trifluoromethyl)-2-pyridyl]methyl]oxamide (35 mg, 80.02 μmol, 10.37% yield).

¹H NMR (600 MHz, dmso) 8 0.81-0.95 (m, 6H), 1.00-1.24 (m, 3H), 1.81-1.97 (m, 1H), 3.66-4.15 (m, 1H), 4.48-5.16 (m, 2H), 7.51-7.73 (m, 2H), 8.05-8.23 (m, 2H), 8.33-8.54 (m, 1H), 8.66-8.94 (m, 3H), 10.20-11.85 (m, 1H).

LCMS (ESI): [M+H]⁺m/z: calcd 438.2; found 438.2; Rt=2.996 min.

### Example 46. Synthesis of N1-(6-amino-5-ethylpyridin-3-yl)-N2-((1R,2R)-2-(difluoromethoxy)cyclopentyl)-N2-((5-(trifluoromethyl)pyridin-2-yl)methyl)oxalamide (Compound 35)

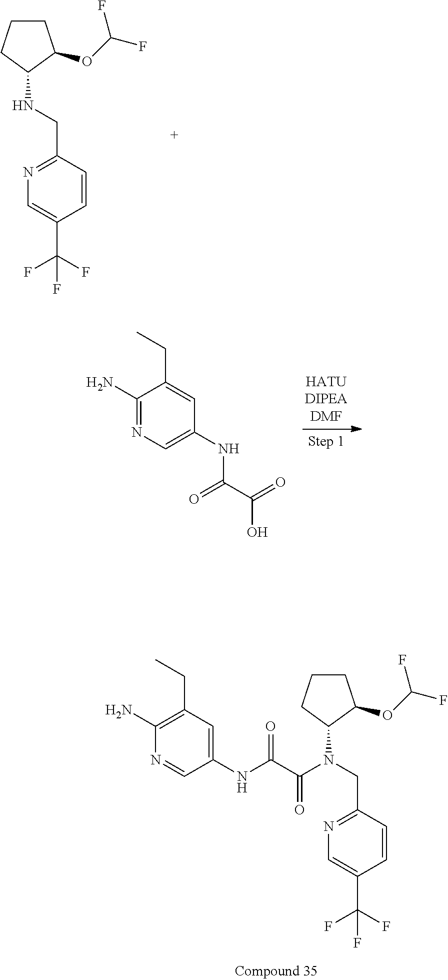 [SMILES: FC(F)O[C@@H]1CCC[C@H]1NCc1ccc(C(F)(F)F)cn1 ; CCc1cc(NC(=O)C(=O)O)cnc1N ; CCc1cc(NC(=O)C(=O)N(Cc2ccc(C(F)(F)F)cn2)[C@@H]2CCC[C@H]2OC(F)F)cnc1N]

(1R,2R)-2-(difluoromethoxy)-N-((5-(trifluoromethyl)pyridin-2-yl)methyl)cyclopentanamine is synthesized by Intermediate 3.

### Step 1: The synthesis of N1-(6-amino-5-ethylpyridin-3-yl)-N2-((1R,2R)-2-(difluoromethoxy)cyclopentyl)-N2-((5-(trifluoromethyl)pyridin-2-yl)methyl)oxalamide

DIPEA (151.36 mg, 1.17 mmol, 203.99 μL) was added to the solution of respective 2-[(6-amino-5-ethyl-3-pyridyl)amino]-2-oxo-acetic acid (0.07 g, 334.61 μmol) and (1R,2R)-2-(difluoromethoxy)-N-[[5-(trifluoromethyl)-2-pyridyl]methyl]cyclopentanamine (103.82 mg, 334.61 μmol) in DMF (9.80 mL). The resulting mixture was stirred for 5 min followed by the addition of HATU (139.95 mg, 368.07 μmol). Then, the reaction mixture was stirred overnight at rt. After the completion of the reaction, monitored by LCMS, the resulting suspension was concentrated under reduced pressure. It was dissolved in EtOAc (20 ml) and washed with water (5 ml), brine (5 ml), dried over Na₂SO₄ and evaporated. The obtained solid was subjected to HPLC (Waters Sunfire C18 20*100 5 mkm column and MeCN+FA as an eluent mixture) to afford N1-(6-amino-5-ethylpyridin-3-yl)-N2-((1R,2R)-2-(difluoromethoxy)cyclopentyl)-N2-((5-(trifluoromethyl)pyridin-2-yl)methyl)oxalamide (16.3 mg, 29.77 μmol, 8.90% yield, HCOOH).

¹H NMR (DMSO-d6, 600 MHz): δ (ppm) 1.08 (m, 3H), 1.67 (m, 4H), 1.92 (m, 2H), 2.33 (m, 2H), 4.44 (m, 1H), 4.72 (m, 2H), 4.97 (m, 1H), 5.67 (m, 2H), 6.58 (m, 1H), 7.49 (m, 2H), 7.99 (m, 1H), 8.17 (m, 1H), 8.88 (m, 1H), 10.38 (m, 1H).

LCMS (ESI): [M+H]⁺m/z: calcd 502.2; found 502.2; Rt=2.946 min.

### Example 47. Synthesis of N1-(5-carbamoyl-6-methoxypyridin-3-yl)-N2-((1R,2R)-2-(difluoromethoxy)cyclopentyl)-N2-((5-(trifluoromethyl)pyridin-2-yl)methyl)oxalamide (Compound 32)

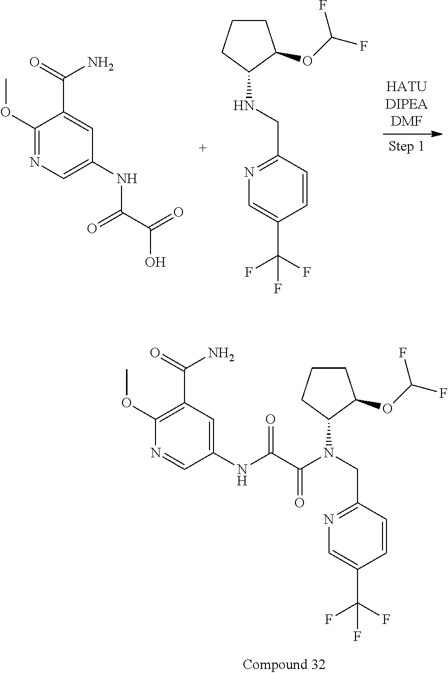 [SMILES: COc1ncc(NC(=O)C(=O)O)cc1C(N)=O ; FC(F)O[C@@H]1CCC[C@H]1NCc1ccc(C(F)(F)F)cn1 ; COc1ncc(NC(=O)C(=O)N(Cc2ccc(C(F)(F)F)cn2)[C@@H]2CCC[C@H]2OC(F)F)cc1C(N)=O]

The synthesis of (1R,2R)-2-(difluoromethoxy)-N-((5-(trifluoromethyl)pyridin-2-yl)methyl)cyclopentan-1-amine is described in Intermediate 3.

### Step 1. The synthesis of N1-(5-carbamoyl-6-methoxypyridin-3-yl)-N2-((1R,2R)-2-(difluoromethoxy)cyclopentyl)-N2-((5-(trifluoromethyl)pyridin-2-yl)methyl)oxalamide

DIPEA (151.30 mg, 1.17 mmol, 203.91 μL) was added to the solution of respective 2-[(5-carbamoyl-6-methoxy-3-pyridyl)amino]-2-oxo-acetic acid (0.08 g, 334.47 μmol) and (1R,2R)-2-(difluoromethoxy)-N-[[5-(trifluoromethyl)-2-pyridyl]methyl]cyclopentanamine (103.77 mg, 334.47 μmol) in DMF (10 mL). The resulting mixture was stirred for 5 min followed by the addition of HATU (139.89 mg, 367.92 μmol). Then, the reaction mixture was stirred overnight at rt. After the completion of the reaction, monitored by LCMS, the resulting suspension was concentrated under reduced pressure. It was dissolved in EtOAc (20 ml) and washed with water (5 ml), brine (5 ml), dried over Na₂SO₄ and evaporated. The obtained solid was subjected to HPLC (Waters Sunfire C18 20*100 5 mkm column and MeCN+FA as an eluent mixture) to afford N1-(5-carbamoyl-6-methoxypyridin-3-yl)-N2-((1R,2R)-2-(difluoromethoxy)cyclopentyl)-N2-((5-(trifluoromethyl)pyridin-2-yl)methyl)oxalamide (11.2 mg, 19.40 μmol, 5.80% yield, HCOOH) as a yellow solid.

¹H NMR (600 MHz, dmso) δ 1.60-2.06 (m, 6H), 3.88-3.96 (m, 3H), 4.28-5.07 (m, 4H), 6.41-6.83 (m, 1H), 7.50-7.77 (m, 3H), 8.12-8.24 (m, 1H), 8.37-8.42 (m, 1H), 8.51-8.57 (m, 1H), 8.81-8.95 (m, 1H), 10.84-11.03 (m, 1H).

LCMS (ESI): [M+H]⁺m/z: calcd 532.2; found 532.0; Rt=2.955 min.

### Example 48. Synthesis of N-(6-amino-5-ethyl-3-pyridyl)-N′-(1,2-dimethylpropyl)-N′-[[5-(trifluoromethyl)-2-pyridyl]methyl]oxamide (Compound 42)

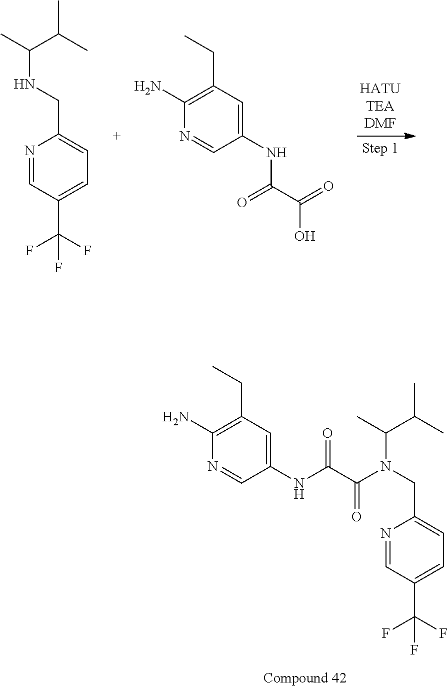 [SMILES: CC(C)C(C)NCc1ccc(C(F)(F)F)cn1 ; CCc1cc(NC(=O)C(=O)O)cnc1N ; CCc1cc(NC(=O)C(=O)N(Cc2ccc(C(F)(F)F)cn2)C(C)C(C)C)cnc1N]

The synthesis of 3-methyl-N-((5-(trifluoromethyl)pyridin-2-yl)methyl) butan-2-amine is described in Intermediate 5.

### Step 1. The synthesis of N-(6-amino-5-ethyl-3-pyridyl)-N′-(1,2-dimethylpropyl)-N′-[[5-(trifluoromethyl)-2-pyridyl]methyl]oxamide

To a solution of 3-methyl-N-[5-(trifluoromethyl)-2-pyridyl]methyl]butan-2-amine (190 mg, 771.51 μmol), 2-[(6-amino-5-ethyl-3-pyridyl)amino]-2-oxo-acetic acid (193.68 mg, 925.81 μmol) and Triethylamine (390.34 mg, 3.86 mmol, 537.66 μL) in DMF (4 mL), HATU (396.02 mg, 1.04 mmol) was added. The resulting mixture was stirred at 40° C. for 4 hr. The reaction mixture was purified by HPLC (Device (Mobile Phase, Column): SYSTEM 40-80% 0-5 min H₂O/MeOH/0.1% NH₄OH, flow: 30 ml/min (loading pump 4 ml/min acetonitrile) target mass 437.47 column: XBridge BEH C18 100×19 mm, 5 μm) to give N-(6-amino-5-ethyl-3-pyridyl)-N′-(1,2-dimethylpropyl)-N′-[5-(trifluoromethyl)-2-pyridyl]methyl]oxamide (162 mg, 370.32 μmol, 48.00% yield) as a light-yellow solid.

¹H NMR (600 MHz, dmso) 8 0.81-0.91 (m, 6H), 1.02-1.19 (m, 6H), 1.77-1.94 (m, 1H), 2.30-2.43 (m, 2H), 3.66-4.13 (m, 1H), 4.46-5.13 (m, 2H), 5.85 (s, 2H), 7.26-7.54 (m, 1H), 7.54-7.68 (m, 1H), 7.85-8.10 (m, 1H), 8.12-8.22 (m, 1H), 8.80-8.93 (m, 1H), 10.19-10.58 (m, 1H).

LCMS (ESI): [M+H]⁺m/z: calcd 438.3; found 438.2; Rt=3.044 min.

### Example 49. Synthesis of N-(4-amino-1H-pyrazolo[4,3-c]pyridin-7-yl)-N′-[(3-chloro-2-pyridyl)methyl]-N′-[(5-fluoro-2-pyridyl)methyl]oxamide (Compound 66)

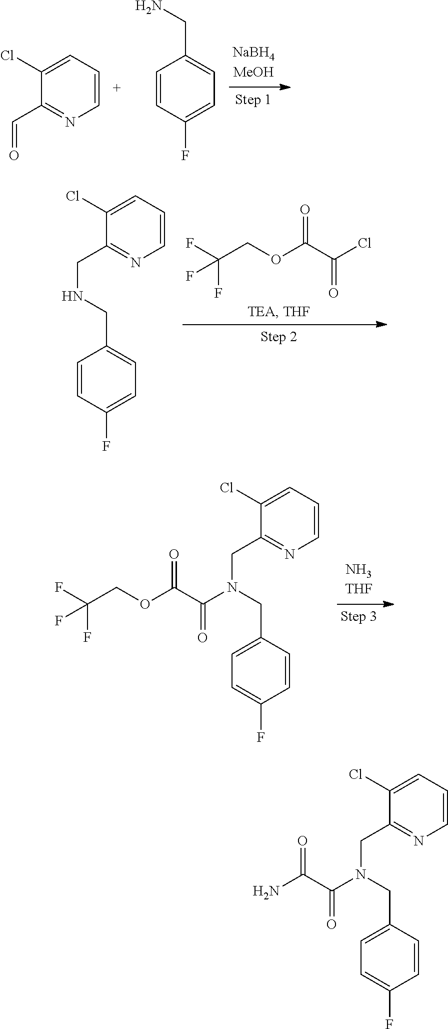

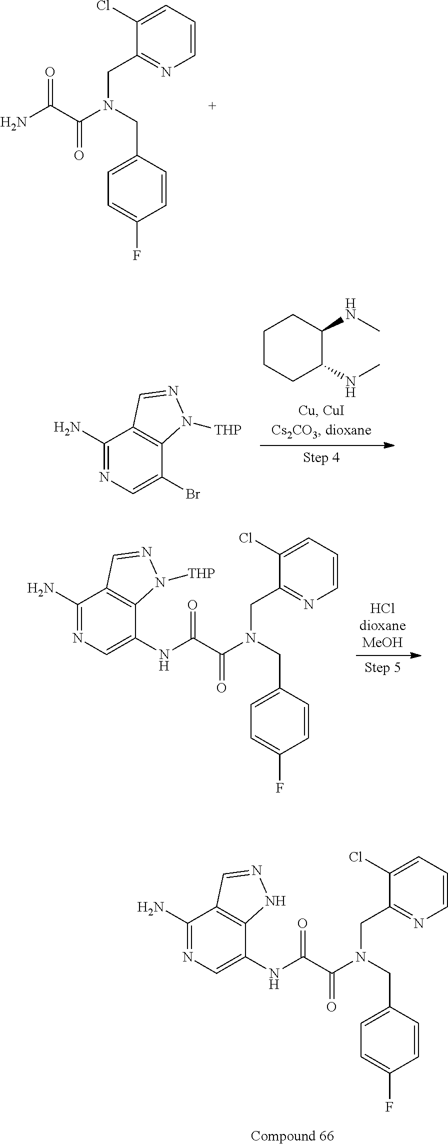

### Step 1: N-[(3-chloro-2-pyridyl)methyl]-1-(4-fluorophenyl)methanamine

A solution of (4-fluorophenyl)methanamine (1 g, 7.99 mmol, 913.24 μL) and 3-chloropyridine-2-carbaldehyde (1.13 g, 7.99 mmol) in Methanol (70 mL) was stirred at 25° C. for 12 h. To this solution, Sodium Borohydride (302.31 mg, 7.99 mmol, 281.48 μL) was added and the resulting mixture was stirred for 2 hr. The solvent was removed in vacuo, the residue was taken up with water (20 mL) and extracted with DCM (3×30 mL). The combined organic layer was washed with brine (20 mL), dried over anhydrous sodium sulfate and evaporated to obtain N-[(3-chloro-2-pyridyl)methyl]-1-(4-fluorophenyl)methanamine (2 g, 7.98 mmol, 99.84% yield) as a yellow oil.

LCMS (ESI): [M+H]⁺m/z: calcd 251.08; found 251.2; Rt=0.825 min.

### Step 2:2,2,2-trifluoroethyl 2-[(3-chloro-2-pyridyl)methyl-[(4-fluorophenyl)methyl]amino]-2-oxo-acetate

To a solution of N-[(3-chloro-2-pyridyl)methyl]-1-(4-fluorophenyl)methanamine (1 g, 3.99 mmol) and Triethylamine (2.02 g, 19.94 mmol, 2.78 mL) in THF (40 mL), 2,2,2-trifluoroethyl 2-chloro-2-oxo-acetate (1.90 g, 9.97 mmol) was added portionwise at 0° C. The resulting mixture was stirred for 2 hr. LCMS showed full conversion of SM. The reaction mixture was directly used for the next step.

LCMS (ESI): [M+H]⁺m/z: calcd 405.07; found 405.0; Rt=1.349 min.

### Step 3: N′-[(3-chloro-2-pyridyl)methyl]-N′-[(4-fluorophenyl)methyl]oxamide

Through a solution of 2,2,2-trifluoroethyl 2-[(3-chloro-2-pyridyl)methyl-[(4-fluorophenyl)methyl]amino]-2-oxo-acetate (1.5 g, 3.71 mmol) in THF (40 mL), ammonia was bubbled during 10 min at 0° C. The formed precipitate was filtered off, washed with THF (30 mL) and the solvent was evaporated in vacuo to give crude product (1.2 g), which was purified by HPLC (Device (Mobile Phase, Column): SYSTEM 25-25-75% 0-1-6 min H₂O/MeOH, flow: 30 mL/min (loading pump 4 mL/min MeOH) target mass 321 column: Chromatorex 18 SNB100-5T 100×19 mm 5 μm) to afford N′-[(3-chloro-2-pyridyl)methyl]-N′-[(4-fluorophenyl)methyl]oxamide (0.9 g, 2.80 mmol, 75.48% yield) as a light-yellow solid.

LCMS (ESI): [M+H]⁺m/z: calcd 322.09; found 322.2; Rt=2.969 min.

### Step 4: N-(4-amino-1-tetrahydropyran-2-yl-pyrazolo[4,3-c]pyridin-7-yl)-N′-[(3-chloro-2-pyridyl)methyl]-N′-[(5-fluoro-2-pyridyl)methyl]oxamide

Copper (1.19 mg, 18.65 μmol), Copper (I) iodide (35.52 mg, 186.49 μmol, 6.32 μL), cesium carbonate (243.05 mg, 745.96 μmol) was added to a stirred solution of N′-[(3-chloro-2-pyridyl)methyl]-N′-[(4-fluorophenyl)methyl]oxamide (0.12 g, 372.98 μmol), 7-bromo-1-tetrahydropyran-2-yl-pyrazolo[4,3-c]pyridin-4-amine (116.37 mg, 391.63 μmol), rac-(1R,2R)—N1,N2-dimethylcyclohexane-1,2-diamine (26.53 mg, 186.49 μmol) in 1,4-dioxane under Ar atmosphere and stirred at 110° C. for 48 hr in closed vial. Reaction mixture was filtered, solid washed with dioxane (2×3 mL), filtrate concentrated to give crude N-(4-amino-1-tetrahydropyran-2-yl-pyrazolo[4,3-c]pyridin-7-yl)-N′-[(3-chloro-2-pyridyl)methyl]-N′-[(5-fluoro-2-pyridyl)methyl]oxamide (0.2 g, 371.08 μmol, 99.49% yield) as a brown solid.

LCMS (ESI): [M+H]⁺m/z: calcd 538.18; found 538.2; Rt=1.244 min.

### Step 5: The synthesis of N-(4-amino-1H-pyrazolo[4,3-c]pyridin-7-yl)-N′-[(3-chloro-2-pyridyl)methyl]-N′-[(5-fluoro-2-pyridyl)methyl]oxamide

Hydrogen chloride solution 4.0M in dioxane (1.35 g, 37.11 mmol, 1.69 mL) was added to a solution of N-(4-amino-1-tetrahydropyran-2-yl-pyrazolo[4,3-c]pyridin-7-yl)-N′-[(3-chloro-2-pyridyl)methyl]-N′-[(5-fluoro-2-pyridyl)methyl]oxamide (0.2 g, 371.08 μmol) in Methanol (6.25 mL) and stirred at 20° C. for 18 hr. Volatiles was evaporated in vacuo, the residue triturated with IPA (5 mL), filtered, washed with IPA (5 mL), and submitted to HPLC (column: Chromatorex 18 SMB100-5T 100×19 mm 5 μm; 0-40% 0-5 min H₂O/ACN/0.1% FA, flow rate: 30 mL/min) to give N-(4-amino-1H-pyrazolo[4,3-c]pyridin-7-yl)-N′-[(3-chloro-2-pyridyl)methyl]-N′-[(5-fluoro-2-pyridyl)methyl]oxamide (26 mg, 51.91 μmol, 13.99% yield, HCOOH) as a yellow gum.

¹H NMR (600 MHz, DMSO-d₆) δ 4.49-4.71 (m, 2H), 4.89-5.17 (m, 2H), 6.90 (s, 2H), 7.10-7.18 (m, 2H), 7.29-7.47 (m, 3H), 7.57-7.73 (m, 1H), 7.80-7.92 (m, 1H), 8.17-8.27 (m, 1H), 8.45-8.55 (m, 1H), 10.23-10.63 (m, 1H), 12.89 (br s, 1H).

LCMS (ESI): [M+H]⁺m/z: calcd 454.13; found 454.2; Rt=2.797 min.

### Example 50. Synthesis of N-(4-amino-1H-pyrazolo[4,3-c]pyridin-7-yl)-N′-[(2-chloro-4-fluoro-phenyl)methyl]-N′-(2-pyridylmethyl)oxamide (Compound 77)

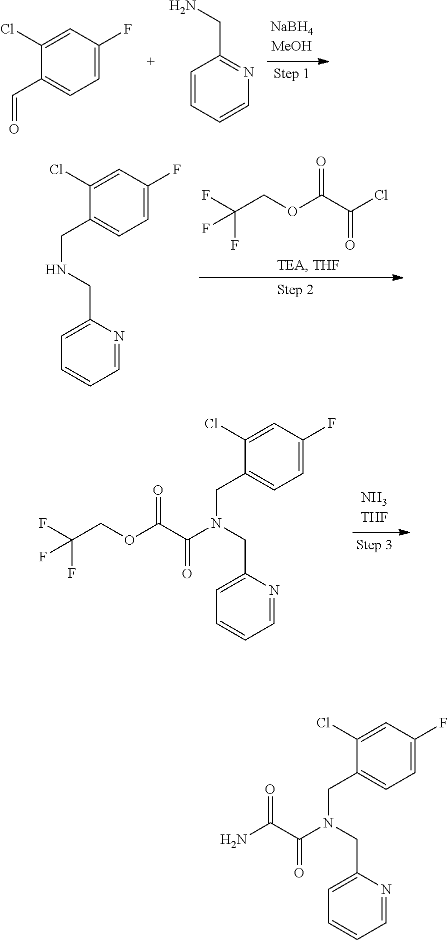

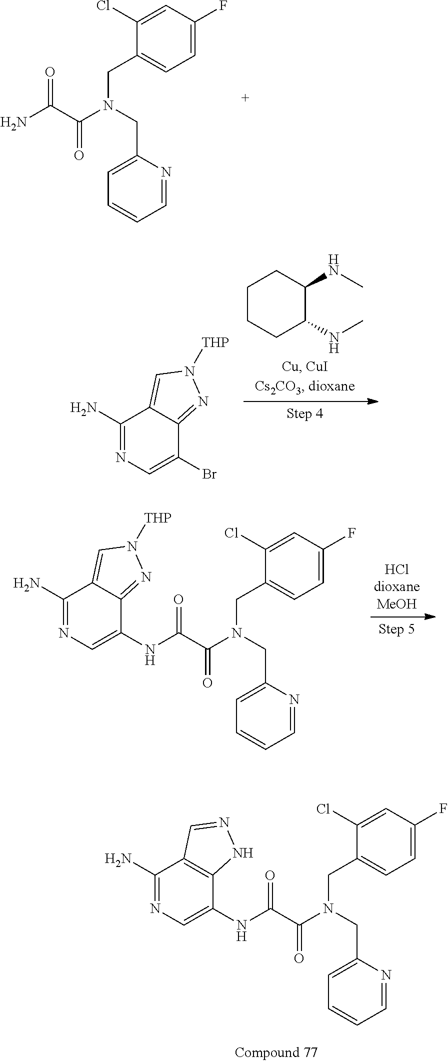

### Step 1:1-(2-chloro-4-fluoro-phenyl)-N-(2-pyridylmethyl)methanamine

A solution of pyridine-2-carbaldehyde (2 g, 18.67 mmol, 1.78 mL) and (2-chloro-4-fluoro-phenyl)methanamine (2.98 g, 18.67 mmol) in MeOH (40 mL) was stirred at 20° C. for 12 hr. To this solution, Sodium Borohydride (777.02 mg, 20.54 mmol, 723.48 μL) was added and the resulting mixture was stirred for 12 hr. The solvent was removed in vacuo, the residue was taken up with water (40 mL) and extracted with DCM (3×20 mL). The combined organic layer was washed with brine (20 mL), dried over anhydrous sodium sulfate and evaporated to obtain 1-(2-chloro-4-fluoro-phenyl)-N-(2-pyridylmethyl)methanamine (3.4 g, 13.56 mmol, 72.63% yield) as a yellow oil.

LCMS (ESI): [M+H]⁺m/z: calcd 251.08; found 251.0; Rt=1.755 min.

### Step 2:2,2,2-trifluoroethyl 2-[(2-chloro-4-fluoro-phenyl)methyl-(2-pyridylmethyl)amino]-2-oxo-acetate

To a solution of 1-(2-chloro-4-fluoro-phenyl)-N-(2-pyridylmethyl)methanamine (2 g, 7.98 mmol) and TEA (1.21 g, 11.97 mmol, 1.67 mL) in THF (40 mL) was added 2,2,2-trifluoroethyl 2-chloro-2-oxo-acetate (1.82 g, 9.57 mmol) dropwise at 0° C. under argon. The reaction mixture was then stirred for 15 hr at r.t., then evaporated in vacuo to give 2,2,2-trifluoroethyl 2-[(2-chloro-4-fluoro-phenyl)methyl-(2-pyridylmethyl)amino]-2-oxo-acetate (2.9 g, 7.17 mmol, 89.81% yield) as a yellow oil.

LCMS (ESI): [M+H]⁺m/z: calcd 405.07; found 405.0; Rt=3.997 min.

### Step 3: N′-[(2-chloro-4-fluoro-phenyl)methyl]-N′-(2-pyridylmethyl)oxamide

2,2,2-trifluoroethyl 2-[(2-chloro-4-fluoro-phenyl)methyl-(2-pyridylmethyl)amino]-2-oxo-acetate (2.9 g, 7.17 mmol) was dissolved in THF (40 mL) and was blow ammonium (2.44 g, 143.30 mmol). The resulting solution was stirred at 20° C. for 12 hr. The resulting mixture was evaporated in vacuo and residue was triturated with THF (2×20 mL), filtered and combined organic was evaporated in vacuo to leave 1.7 g of crude product which was purification by column chromatography on silica gel using CHCl₃/CH₃CN gradient (10-100% MTBE) to afford N′-[(2-chloro-4-fluoro-phenyl)methyl]-N′-(2-pyridylmethyl)oxamide (0.6 g, 1.86 mmol, 26.03% yield) as a yellow oil.

LCMS (ESI): [M+H]⁺m/z: calcd 322.08; found 322.0; Rt=0.926 min.

### Step 4: N-(4-amino-1-tetrahydropyran-2-yl-pyrazolo[4,3-c]pyridin-7-yl)-N′-[(2-chloro-4-fluoro-phenyl)methyl]-N′-(2-pyridylmethyl)oxamide

7-bromo-1-tetrahydropyran-2-yl-pyrazolo[4,3-c]pyridin-4-amine (277.08 mg, 932.45 μmol), N′-[(2-chloro-4-fluoro-phenyl)methyl]-N′-(2-pyridylmethyl)oxamide (0.3 g, 932.45 μmol), Cu(2.96 mg, 46.62 μmol), CuI (177.58 mg, 932.45 μmol, 31.60 μL), cesium carbonate (455.72 mg, 1.40 mmol) and (1R,2R)—N1,N2-dimethylcyclohexane-1,2-diamine (159.16 mg, 1.12 mmol) were mixed in dioxane (6 mL), purged with Ar for 15 minutes and then heated in the sealed tube at 110° C. for 48 hr. Final mixture was filtered and dioxane was evaporated in vacuo. The crude product 0.4 g was purified by RP-HPLC (column: YMC Triart C18 5 μm 130A; 35-35-50% 0-1.5-5 min H₂O/CH₃CN/0.1% NH₄OH, flow: 30 mL/min) to give N-(4-amino-1-tetrahydropyran-2-yl-pyrazolo[4,3-c]pyridin-7-yl)-N′-[(2-chloro-4-fluoro-phenyl)methyl]-N′-(2-pyridylmethyl)oxamide (0.0653 g, 121.38 μmol, 13.02% yield) as a yellow gum.

LCMS (ESI): [M+H]⁺m/z: calcd 538.2; found 538.2; Rt=1.469 min.

### Step 5: The synthesis of N-(4-amino-1H-pyrazolo[4,3-c]pyridin-7-yl)-N′-[(2-chloro-4-fluoro-phenyl)methyl]-N′-(2-pyridylmethyl)oxamide

Hydrogen chloride, 4M in 1,4-dioxane, 99% (2.40 g, 65.82 mmol, 3 mL) was added to a solution of N-(4-amino-1-tetrahydropyran-2-yl-pyrazolo[4,3-c]pyridin-7-yl)-N′-[(2-chloro-4-fluoro-phenyl)methyl]-N′-(2-pyridylmethyl)oxamide (67.56 mg, 125.58 μmol) in MeOH (7.99 mL). The reaction mixture was stirred at 20° C. for 8 hr, then evaporated. The residue was purified by RP-HPLC (column: XBridge BEH18 SMB100-BT 100×19 mm; 35-35-60% 0-1.5-5 min H₂O/CH₃CN/0.1% NH₄OH, flow: 30 mL/min) to give N-(4-amino-1H-pyrazolo[4,3-c]pyridin-7-yl)-N′-[(2-chloro-4-fluoro-phenyl)methyl]-N′-(2-pyridylmethyl)oxamide (33.30 mg, 73.37 μmol, 58.43% yield) as a white solid.

¹H NMR (600 MHz, DMSO-d₆) δ 4.42-5.02 (m, 4H), 6.64 (s, 2H), 6.76-7.62 (m, 6H), 7.62-7.81 (m, 2H), 8.12-8.21 (m, 1H), 8.48-8.53 (m, 1H), 10.16 (s, 1H).

LCMS (ESI): [M+H]⁺m/z: calcd 454.13; found 454.2; Rt=2.524 min.

### Example 51. Synthesis of N-(4-amino-1H-pyrazolo[4,3-c]pyridin-7-yl)-N′-benzyl-N′-[(3-chloro-5-fluoro-2-pyridyl)methyl]oxamide (Compound 65)

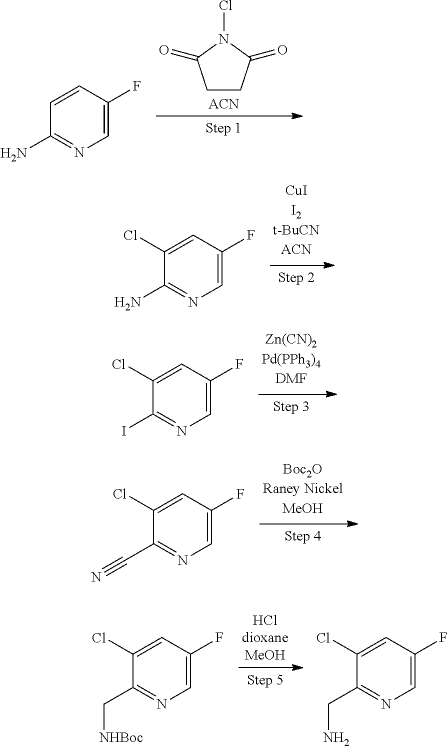

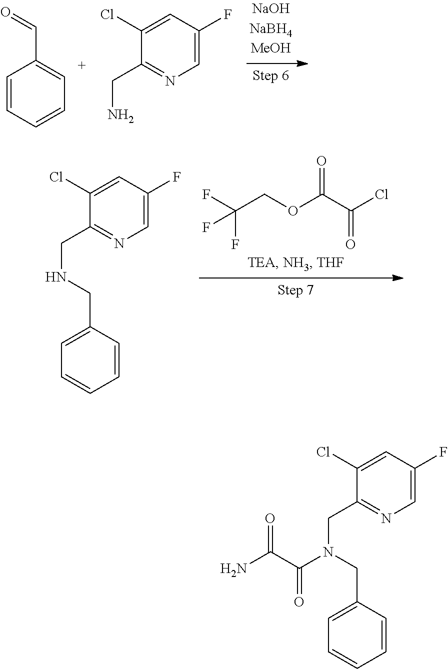

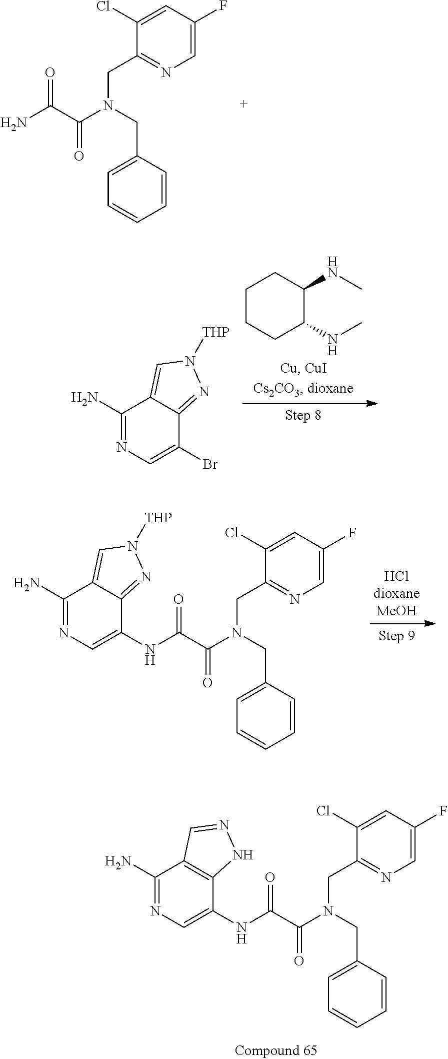

### Step 1:3-chloro-5-fluoro-pyridin-2-amine

A mixture of 5-fluoropyridin-2-amine (10 g, 89.20 mmol) and 1-chloropyrrolidine-2,5-dione (13.10 g, 98.12 mmol, 7.94 mL) in acetonitrile (200 mL) was stirred with a reflux condenser at 80° C. for 24 hr. The reaction mixture was cooled down and concentrated in vacuo. The residue was diluted with water (150 mL) and the precipitate was filtered, washed with water (5×20 mL) and air dried to afford 3-chloro-5-fluoro-pyridin-2-amine (8 g, 54.59 mmol, 61.20% yield) as a brown solid which was used directly in the next step.

LCMS (ESI): [M+H]⁺m/z: calcd 147.01; found 147.0; Rt=0.801 min.

### Step 2:3-chloro-5-fluoro-2-iodo-pyridine

A solution of 3-chloro-5-fluoro-pyridin-2-amine (8 g, 54.59 mmol) in acetonitrile (60 mL) was added dropwise over 0.5 hr period to a preheated to 60° C. mixture of Copper (I) iodide (12.48 g, 65.51 mmol, 2.22 mL), iodine (20.78 g, 81.88 mmol) and tert-butyl nitrite (22.52 g, 218.36 mmol, 25.97 mL) in acetonitrile (150 mL) with stirring. The resulting mixture was stirred with a reflux condenser at 80° C. for 24 hr, then cooled down and concentrated in vacuo. The residue was diluted with MTBE (500 mL) and 10% aqueous NaHSO₃ solution (400 mL). The resulting mixture was stirred vigorously for 0.5 hr and then filtered. The filtercake was washed with MTBE (3×50 mL) and discarded. The combined filtrate was transferred to a separatory funnel, the organic layer was separated, dried over sodium sulfate, and concentrated in vacuo to afford crude product as brown solid, which was purified by column chromatography on silica gel using chloroform/MTBE gradient (0-100% MTBE) to afford 3-chloro-5-fluoro-2-iodo-pyridine (2.6 g, 10.10 mmol, 18.50% yield) as a yellow solid.

### Step 3:3-chloro-5-fluoro-pyridine-2-carbonitrile

A mixture of 3-chloro-5-fluoro-2-iodo-pyridine (2.6 g, 10.10 mmol) and Zinc Cyanide (0.74 g, 6.30 mmol, 399.57 μL) in DMF (35 mL) was evacuated and then backfilled with argon. This operation was repeated two times, then Tetrakis(triphenylphosphine) palladium (0)(0.9 g, 776.14 μmol) was added under argon. The reaction mixture was stirred under argon at 100° C. for 18 hr, then cooled down and concentrated in vacuo. The residue was diluted with water (40 mL) and MTBE (60 mL). The resulting mixture was stirred vigorously for 15 min., and then filtered. The filtercake was washed with MTBE (3×20 mL) and discarded. The combined filtrate was transferred to a separatory funnel, the organic layer was separated, washed with water (2×25 mL), dried over anhydrous sodium sulfate and concentrated in vacuo to leave crude product (1.5 g), which was purified by column chromatography on silica gel using chloroform/acetonitrile gradient (0-100% acetonitrile) to afford 3-chloro-5-fluoro-pyridine-2-carbonitrile (600 mg, 3.83 mmol, 37.95% yield) as a beige solid.

### Step 4: tert-butyl N-[(3-chloro-5-fluoro-2-pyridyl)methyl]carbamate

A mixture of 3-chloro-5-fluoro-pyridine-2-carbonitrile (600 mg, 3.83 mmol), di-tert-butyl dicarbonate (1.25 g, 5.75 mmol, 1.32 mL) and Raney Nickel©800, slurry, in H₂O, active catalyst (0.4 g, 4.67 mmol) in methanol (40 mL) was hydrogenated under 10 atm. hydrogen pressure in autoclave at 50° C. for 12 hr. The catalyst was carefully filtered off, the filtrate was concentrated in vacuo to afford tert-butyl N-[(3-chloro-5-fluoro-2-pyridyl)methyl]carbamate (1.3 g, crude) as a yellow gum which was used directly in the next step.

LCMS (ESI): [M-Boc+H]⁺m/z: calcd 204.08; found 205.0; Rt=1.105 min.

### Step 5: (3-chloro-5-fluoro-2-pyridyl)methanamine

Hydrogen chloride solution 4.0M in dioxane (42.00 g, 160.12 mmol, 40 mL, 13.9% purity) was added in one portion to a stirred solution of tert-butyl N-[(3-chloro-5-fluoro-2-pyridyl)methyl]carbamate (1.3 g, 4.99 mmol) in methanol (38.98 mL) at 25° C. The resulting solution was stirred at 25° C. for 2 hr and then concentrated in vacuo to afford (3-chloro-5-fluoro-2-pyridyl)methanamine (700 mg, 3.00 mmol, 60.12% yield, 2HCl) as a light-yellow solid.

LCMS (ESI): [M+H]⁺m/z: calcd 161.03; found 161.0; Rt=0.310 min.

### Step 6: N-[(3-chloro-5-fluoro-2-pyridyl)methyl]-1-phenyl-methanamine

(3-chloro-5-fluoro-2-pyridyl)methanamine (700 mg, 3.00 mmol, 2HCl) was added to a stirred solution of Sodium hydroxide, pearl (251.80 mg, 6.30 mmol, 118.22 μL) in methanol (24.89 mL) at 25° C. After 5 min., benzaldehyde (477.21 mg, 4.50 mmol) was added at 25° C. and the reaction mixture was stirred at 25° C. for 6 hr, then cooled to −10° C. using crushed ice/NaCl bath, and Sodium Borohydride (170.13 mg, 4.50 mmol, 158.40 μL) was added in one portion. The reaction mixture was allowed to warm to r.t. and stirred for 0.5 hr. The reaction mixture was concentrated in vacuo, the residue was diluted with water (20 mL) and extracted with MTBE (2×20 mL). The combined organic extracts were dried over anhydrous sodium sulfate and concentrated in vacuo to afford N-[(3-chloro-5-fluoro-2-pyridyl)methyl]-1-phenyl-methanamine (1 g, crude) as a brown gum which was used directly in the next step.

LCMS (ESI): [M+H]⁺m/z: calcd 251.08; found 251.0; Rt=0.852 min.

### Step 7: N′-benzyl-N′-[(3-chloro-5-fluoro-2-pyridyl)methyl]oxamide

2,2,2-trifluoroethyl 2-chloro-2-oxo-acetate (1.14 g, 5.98 mmol) was added slowly to a cooled to −10° C. mixture of N-[(3-chloro-5-fluoro-2-pyridyl)methyl]-1-phenyl-methanamine (1 g, 3.99 mmol) and triethyl amine (2.02 g, 19.94 mmol, 2.78 mL) in THF (60 mL). The resulting mixture was allowed to warm to 25° C. and stirred for 1 hr. Then gaseous ammonia (1.70 g, 99.72 mmol) was vigorously bubbled through it at 25° C. for 0.5 hr. The resulting mixture was filtered to remove ammonium chloride, the filtercake was washed with THF (2×20 mL) and discarded. The combined filtrate was concentrated in vacuo to afford crude product (1.1 g), which was purified by reverse phase HPLC (column: Chromatorex 18 SMB100-5T 100×19 mm 5 μm; mobile phase: 45-45-70% 0-1-5 min H₂O/MeOH; flow rate: 30 mL/min (loading pump 4 mL/min MeOH) to afford N′-benzyl-N′-[(3-chloro-5-fluoro-2-pyridyl)methyl]oxamide (426 mg, 1.32 mmol, 33.19% yield) as a yellow gum.

LCMS (ESI): [M+H]⁺m/z: calcd 322.09; found 322.2; Rt=2.627 min.

### Step 8: N-(4-amino-2-tetrahydropyran-2-yl-pyrazolo[4,3-c]pyridin-7-yl)-N′-benzyl-N′-[(3-chloro-5-fluoro-2-pyridyl)methyl]oxamide

A mixture of N′-benzyl-N′-[(3-chloro-5-fluoro-2-pyridyl)methyl]oxamide (200 mg, 621.63 μmol), 7-bromo-2-tetrahydropyran-2-yl-pyrazolo[4,3-c]pyridin-4-amine (277.08 mg, 932.45 μmol), copper (5 mg, 78.68 μmol), Copper (I) iodide (150 mg, 787.61 μmol, 26.69 μL), cesium carbonate (324.06 mg, 994.61 μmol) and rac-(1R,2R)—N1,N2-dimethylcyclohexane-1,2-diamine (150 mg, 1.05 mmol) in 1,4-dioxane (7 mL) was stirred in a sealed vial under argon at 105° C. for 18 hr. The resulting mixture was cooled down and filtered. The filtercake was washed successively with THF (2×5 mL) and dichloromethane (3×5 mL). The combined filtrate was concentrated in vacuo to afford N-(4-amino-2-tetrahydropyran-2-yl-pyrazolo[4,3-c]pyridin-7-yl)-N′-benzyl-N′-[(3-chloro-5-fluoro-2-pyridyl)methyl]oxamide (1.1 g, crude) as a brown gum which was used directly in the next step.

LCMS (ESI): [M+H]⁺m/z: calcd 538.18; found 538.2; Rt=2.870 min.

### Step 9: The synthesis of N-(4-amino-1H-pyrazolo[4,3-c]pyridin-7-yl)-N′-benzyl-N′-[(3-chloro-5-fluoro-2-pyridyl)methyl]oxamide

Hydrogen chloride solution 4.0M in dioxane (5.25 g, 20.02 mmol, 5 mL, 13.9% purity) was added to a stirred solution of crude from previous step N-(4-amino-2-tetrahydropyran-2-yl-pyrazolo[4,3-c]pyridin-7-yl)-N′-benzyl-N′-[(3-chloro-5-fluoro-2-pyridyl)methyl]oxamide (1.1 g, 2.04 mmol) in methanol (5 mL) at 25° C. The resulting solution was stirred at 25° C. for 2 hr, then concentrated to dryness in vacuo and the residue was submitted to reverse phase HPLC (column: Chromatorex 18 SMB 100-5T 100×19 mm 5 μm; mobile phase: 20-45% 0-5 min H₂O/ACN/0.1% FA; flow: 30 mL/min (loading pump 4 mL/min water)) to afford N-(4-amino-1H-pyrazolo[4,3-c]pyridin-7-yl)-N′-benzyl-N′-[(3-chloro-5-fluoro-2-pyridyl)methyl]oxamide (67 mg, 134.03 μmol, 6.56% yield, HCOOH) as a light-brown solid.

¹H NMR (500 MHz, DMSO-d₆) δ 4.61-4.68 (m, 2H), 4.91-5.09 (m, 2H), 6.74-6.90 (m, 2H), 7.24-7.28 (m, 1H), 7.31-7.39 (m, 4H), 7.55-7.74 (m, 1H), 7.99-8.10 (m, 1H), 8.15-8.21 (m, 1H), 8.48-8.64 (m, 1H), 10.38-10.58 (m, 1H), 12.51-13.36 (m, 2H).

LCMS (ESI): [M+H]⁺m/z: calcd 454.13; found 454.2; Rt=2.253 min.

### Example 52. Synthesis of N-(4-amino-1HI-pyrazolo[4,3-c]pyridin-7-yl)-N′-[(2-chlorophenyl)methyl]-N′-[(5-fluoro-2-pyridyl)methyl]oxamide (Compound 67)

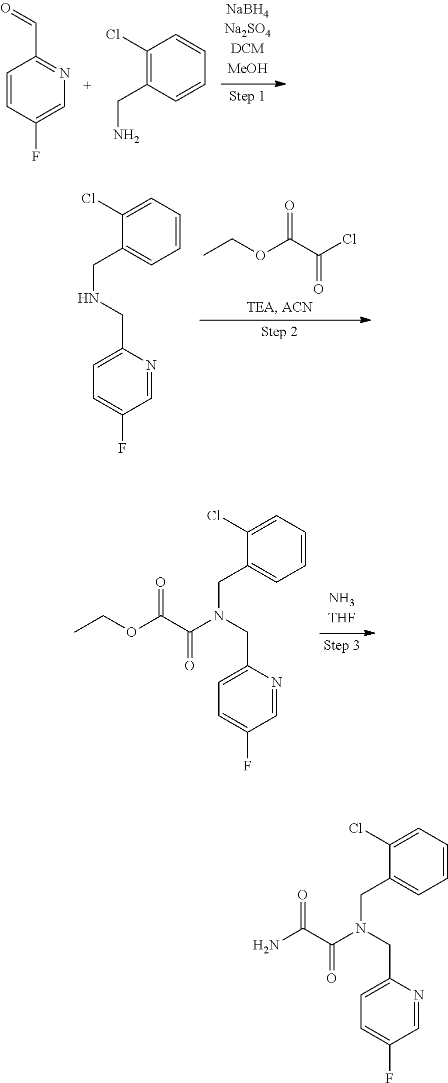

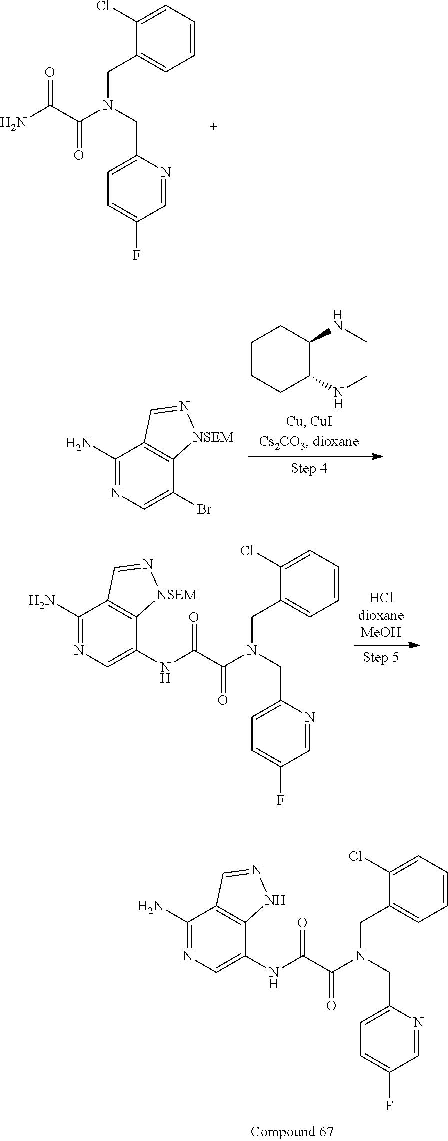

### Step 1:1-(2-chlorophenyl)-N-[(5-fluoro-2-pyridyl)methyl]methanamine

5-fluoropyridine-2-carbaldehyde (0.85 g, 6.79 mmol), Sodium sulfate, anhydrous (965.10 mg, 6.79 mmol, 360.11 μL) and (2-chlorophenyl)methanamine (962.09 mg, 6.79 mmol, 822.30 μL) were mixed in DCM at 20° C. The resulting mixture was stirred at 20° C. for 12 hr, then filtered and concentrated in vacuo. The residue was dissolved in methanol, cooled to 5° C. and Sodium Borohydride (282.74 mg, 7.47 mmol, 263.26 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford 1-(2-chlorophenyl)-N-[(5-fluoro-2-pyridyl)methyl]methanamine (1.5 g, crude) as a light-yellow liquid.

LCMS (ESI): [M+H]⁺m/z: calcd 251.08; found 251.0; Rt=0.708 min.

### Step 2: ethyl 2-[(2-chlorophenyl)methyl-[(5-fluoro-2-pyridyl)methyl]amino]-2-oxo-acetate

1-(2-chlorophenyl)-N-[(5-fluoro-2-pyridyl)methyl]methanamine (1.5 g, 4.79 mmol) and TEA (968.72 mg, 9.57 mmol, 1.33 mL) were dissolved in acetonitrile (20.06 mL), cooled with ice-water bath and then ethyl 2-chloro-2-oxoacetate (849.59 mg, 6.22 mmol, 695.25 μL) was added. Solution was stirred overnight at 20° C. The RM was concentrated in vacuo, redissolved in DCM (40 mL) and washed with water twice. Organic phase was dried over anhydrous sodium sulfate and concentrated in vacuo to give ethyl 2-[(2-chlorophenyl)methyl-[(5-fluoro-2-pyridyl)methyl]amino]-2-oxo-acetate (1.9 g, crude) as a brown liquid.

LCMS (ESI): [M+H]⁺m/z: calcd 351.09; found 351.0; Rt=1.236 min.

### Step 3: N′-[(2-chlorophenyl)methyl]-N′-[(5-fluoro-2-pyridyl)methyl]oxamide

Ethyl 2-[(2-chlorophenyl)methyl-[(5-fluoro-2-pyridyl)methyl]amino]-2-oxo-acetate (1.9 g, 4.06 mmol) was dissolved in MeOH (20 mL) saturated with NH3 (4.06 mmol) and stirred overnight. The RM was concentrated, redissolved in DCM, washed with water, dried over anhydrous sodium sulfate and concentrated in vacuo to give N′-[(2-chlorophenyl)methyl]-N′-[(5-fluoro-2-pyridyl)methyl]oxamide (1.4 g, crude) as a brown solid.

LCMS (ESI): [M+H]⁺m/z: calcd 322.08; found 322.0; Rt=1.105 min.

### Step 4: N-[4-amino-1-(2-trimethylsilylethoxymethyl)pyrazolo[4,3-c]pyridin-7-yl]-N′-[(2-chlorophenyl)methyl]-N′-[(5-fluoro-2-pyridyl)methyl]oxamide

Copper (3.04 mg, 47.87 μmol), Copper (I) iodide (27.35 mg, 143.60 μmol, 4.87 μL), cesium carbonate (311.91 mg, 957.32 μmol) were added to a stirred solution of N′-[(2-chlorophenyl)methyl]-N′-[(5-fluoro-2-pyridyl)methyl]oxamide (220 mg, 478.66 μmol), 7-bromo-1-(2-trimethylsilylethoxymethyl)pyrazolo[4,3-c]pyridin-4-amine (197.18 mg, 574.39 μmol) and rac-(1R,2R)—N1,N2-dimethylcyclohexane-1,2-diamine (34.04 mg, 239.33 μmol) in 1,4-dioxane (5.00 mL) under Ar atmosphere and stirred at 100° C. for 12 hr in closed vial. The reaction mixture was filtered and evaporated in vacuo to give N-[4-amino-1-(2-trimethylsilylethoxymethyl)pyrazolo[4,3-c]pyridin-7-yl]-N′-[(2-chlorophenyl)methyl]-N′-[(5-fluoro-2-pyridyl)methyl]oxamide (0.45 g, crude) as a brown solid.

LCMS (ESI): [M+H]⁺m/z: calcd 584.2; found 584.1; Rt=1.556 min.

### Step 5: The synthesis of N-(4-amino-1H-pyrazolo[4,3-c]pyridin-7-yl)-N′-[(2-chlorophenyl)methyl]-N′-[(5-fluoro-2-pyridyl)methyl]oxamide

N-[4-amino-1-(2-trimethylsilylethoxymethyl)pyrazolo[4,3-c]pyridin-7-yl]-N′-[(2-chlorophenyl)methyl]-N′-[(5-fluoro-2-pyridyl)methyl]oxamide (0.45 g, 346.68 μmol) was dissolved in Dioxane (4 mL) saturated with HCl (10% by weight). MeOH (4 mL) was added for better solubility. The RM was stirred for 12 hr at 20° C. then concentrated in vacuo. The residue was dissolved in 3 mL of MeOH and subjected to HPLC (Device (Mobile Phase, Column): 2-2-6 min 30-55% MeOH+FA flow 30 mL/min) to afford N-(4-amino-1H-pyrazolo[4,3-c]pyridin-7-yl)-N′-[(2-chlorophenyl)methyl]-N′-[(5-fluoro-2-pyridyl)methyl]oxamide (72 mg, 144.03 μmol, 41.55% yield, HCOOH) was obtained.

¹H NMR (600 MHz, DMSO-d₆) δ 4.47-4.76 (m, 2H), 4.76-5.15 (m, 2H), 7.23-7.36 (m, 3H), 7.37-7.53 (m, 3H), 7.56-7.81 (m, 3H), 8.30-8.45 (m, 1H), 8.46-8.52 (m, 1H), 10.72 (s, 1H), 13.37 (br s, 1H).

LCMS (ESI): [M+H]⁺m/z: calcd 454.13; found 454.2; Rt=2.725 min.

### Example 53. Synthesis of (R)—N¹-(4-amino-1H-pyrazolo[4,3-c]pyridin-7-yl)-N2-(2-methylbutyl)-N2-((5-(trifluoromethyl)pyridin-2-yl)methyl)oxalamide (Compound 75)

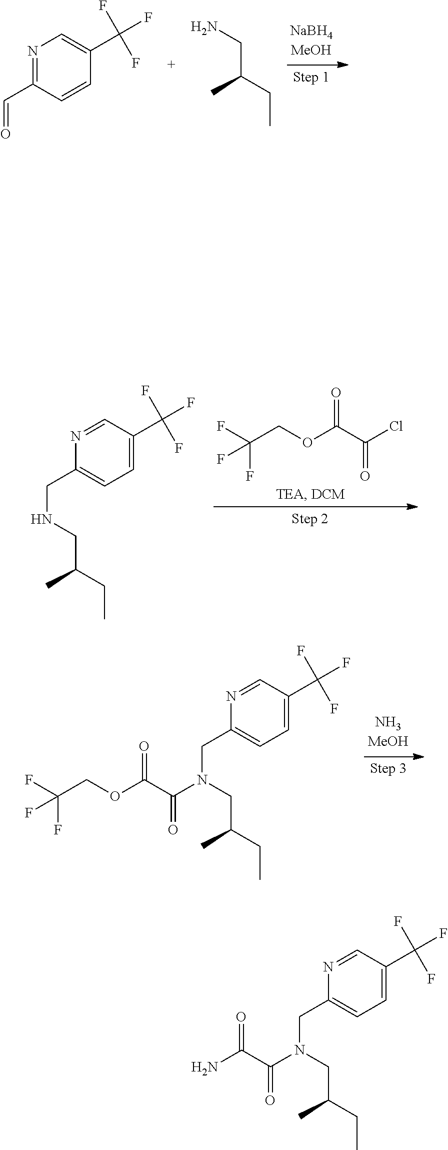

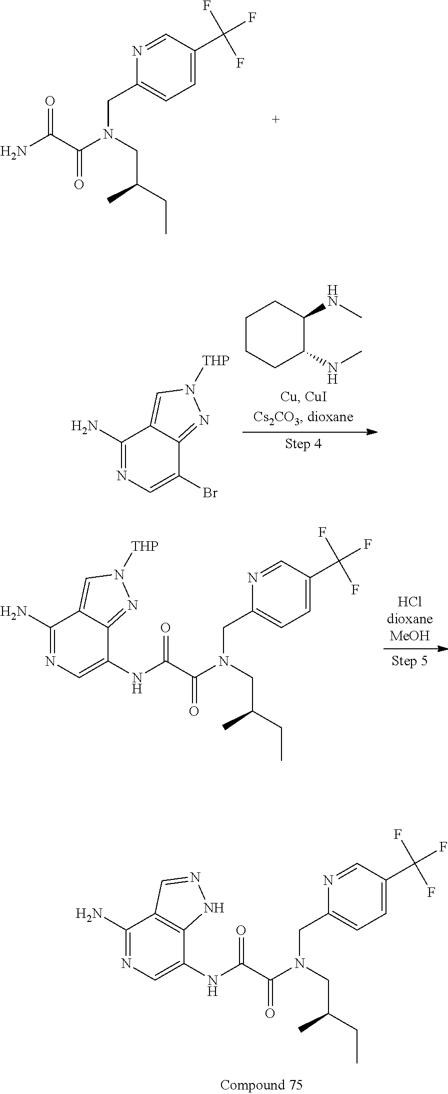

### Step 1: (R)-2-methyl-N-((5-(trifluoromethyl)pyridin-2-yl)methyl) butan-1-amine

(R)-2-methylbutan-1-amine (199.11 mg, 2.28 mmol) were added to the solution of 5-(trifluoromethyl)pyridine-2-carbaldehyde (0.4 g, 2.28 mmol) in Methanol (8 mL). The resulting mixture was stirred at 60° C. for 1 hour before Sodium Borohydride (129.62 mg, 3.43 mmol, 120.69 μL) was added portions thereto. After that, stirring was continued for 15 hr. Then, solvent was removed under reduced pressure and residue was partitioned between 10% aq. K₂CO₃ solution (20 mL) and DCM (20 mL). Organic layer was separated, dried over anhydrous sodium sulfate and concentrated under reduced pressure to afford (R)-2-methyl-N-((5-(trifluoromethyl)pyridin-2-yl)methyl) butan-1-amine (470 mg, crude) as a yellow oil.

LCMS (ESI): [M+H]⁺m/z: calcd 247.14; found 247.2; Rt=0.947 min.

### Step 2: (R)-2,2,2-trifluoroethyl 2-((2-methylbutyl)((5-(trifluoromethyl)pyridin-2-yl)methyl)amino)-2-oxoacetate

(R)-2-methyl-N-((5-(trifluoromethyl)pyridin-2-yl)methyl) butan-1-amine (470 mg, 1.91 mmol) and Triethylamine (289.68 mg, 2.86 mmol, 399.00 μL) were dissolved in DCM (10 mL) and the resulting mixture was cooled to −5° C. A solution of 2,2,2-trifluoroethyl 2-chloro-2-oxo-acetate (381.75 mg, 2.00 mmol) in DCM (2 mL) was added dropwise at −5° C. and the resulting mixture was allowed to warm to room temperature, and stirred overnight. Water (20 mL) was added and an organic layer was separated. The aqueous layer was extracted with DCM (20 mL) and combined organic layers were dried over anhydrous sodium sulfate, filtered, and concentrated in vacuo to obtain (R)-2,2,2-trifluoroethyl 2-((2-methylbutyl)((5-(trifluoromethyl)pyridin-2-yl)methyl)amino)-2-oxoacetate (725 mg, crude) as a light-yellow oil.

LCMS (ESI): [M+H]⁺m/z: calcd 401.13; found 401.2; Rt=1.412 min.

### Step 3: (R)—N¹-(2-methylbutyl)-N1-((5-(trifluoromethyl)pyridin-2-yl)methyl)oxalamide

A solution of (R)-2,2,2-trifluoroethyl 2-((2-methylbutyl)((5-(trifluoromethyl)pyridin-2-yl)methyl)amino)-2-oxoacetate (725 mg, 1.81 mmol) in Methanol/NH3 (20 mL) was stirred at 25° C. for 18 hr. The solvent was evaporated to obtain (R)—N¹-(2-methylbutyl)-N1-((5-(trifluoromethyl)pyridin-2-yl)methyl)oxalamide (602 mg, crude) as a yellow gum.

LCMS (ESI): [M+H]⁺m/z: calcd 318.15; found 318.2; Rt=1.288 min.

### Step 4: N1-(4-amino-2-(tetrahydro-2H-pyran-2-yl)-2H-pyrazolo[4,3-c]pyridin-7-yl)-N2-((R)-2-methylbutyl)-N2-((5-(trifluoromethyl)pyridin-2-yl)methyl)oxalamide

To an 8 ml vial (R)—N¹-(2-methylbutyl)-N1-((5-(trifluoromethyl)pyridin-2-yl)methyl)oxalamide (150 mg, 472.73 μmol), 7-bromo-2-tetrahydropyran-2-yl-pyrazolo[4,3-c]pyridin-4-amine (140.47 mg, 472.73 μmol), Copper (1.50 mg, 23.64 μmol), Copper (I) iodide (45.02 mg, 236.36 μmol, 8.01 μL), rac-(1R,2R)—N1,N2-dimethylcyclohexane-1,2-diamine (50.43 mg, 354.55 μmol), Cesium carbonate (308.05 mg, 945.46 μmol) and Dioxane (8.01 mL) were charged and the resulting mixture was splurged with argon for 5 min. The vial was sealed and heated at 100° C. for 48 hr. The reaction mixture was cooled, diluted with MeOH (5 mL), filtered and evaporated to obtain N1-(4-amino-2-(tetrahydro-2H-pyran-2-yl)-2H-pyrazolo[4,3-c]pyridin-7-yl)-N2-((R)-2-methylbutyl)-N2-((5-(trifluoromethyl)pyridin-2-yl)methyl)oxalamide (420 mg, crude) as a brown gum which was used in the next step without further purification.

LCMS (ESI): [M+H]⁺m/z: calcd 534.25; found 534.2; Rt=1.220 min.

### Step 5: The synthesis of (R)—N¹-(4-amino-1H-pyrazolo[4,3-c]pyridin-7-yl)-N2-(2-methylbutyl)-N2-((5-(trifluoromethyl)pyridin-2-yl)methyl)oxalamide

N1-(4-amino-2-(tetrahydro-2H-pyran-2-yl)-2H-pyrazolo[4,3-c]pyridin-7-yl)-N2-((R)-2-methylbutyl)-N2-((5-(trifluoromethyl)pyridin-2-yl)methyl)oxalamide (420 mg, 788.65 μmol) was dissolved in Methanol (2 mL) and Dioxane/HCl (2 mL). The resulting mixture was stirred overnight. The reaction mixture was concentrated in vacuo. The residue was purified by HPLC (40-70% 2-10 min H₂O/ACN/0.1NH4OH flow 30 mL/min ((loading pump 4 mL ACN); target mass 450; column: Chromatorex C18 SMB100-5T 100×19 mm, 5 microM) to obtain (R)—N¹-(4-amino-1H-pyrazolo[4,3-c]pyridin-7-yl)-N2-(2-methylbutyl)-N2-((5-(trifluoromethyl)pyridin-2-yl)methyl)oxalamide (9.2 mg, 20.52 μmol, 2.60% yield) as a light-yellow solid.

¹H NMR (600 MHz, DMSO-d₆) δ 0.71-0.90 (m, 7H), 0.98-1.14 (m, 1H), 1.31-1.44 (m, 1H), 1.70-1.86 (m, 1H), 3.22 (dd, 1H), 3.45-3.60 (m, 1H), 4.70-4.84 (m, 1H), 5.00-5.12 (m, 1H), 6.50-7.13 (m, 2H), 7.47-7.81 (m, 2H), 8.08-8.30 (m, 2H), 8.76-9.03 (m, 1H), 9.46-10.69 (m, 1H), 12.71 (s, 1H).

LCMS (ESI): [M+H]⁺m/z: calcd 450.21; found 450.0; Rt=1.135 min.

### Example 54. Synthesis of(S)-N1-(4-amino-1H-pyrazolo[4,3-c]pyridin-7-yl)-N2-(2-methylbutyl)-N2-((5-(trifluoromethyl)pyridin-2-yl)methyl)oxalamide (Compound 64)

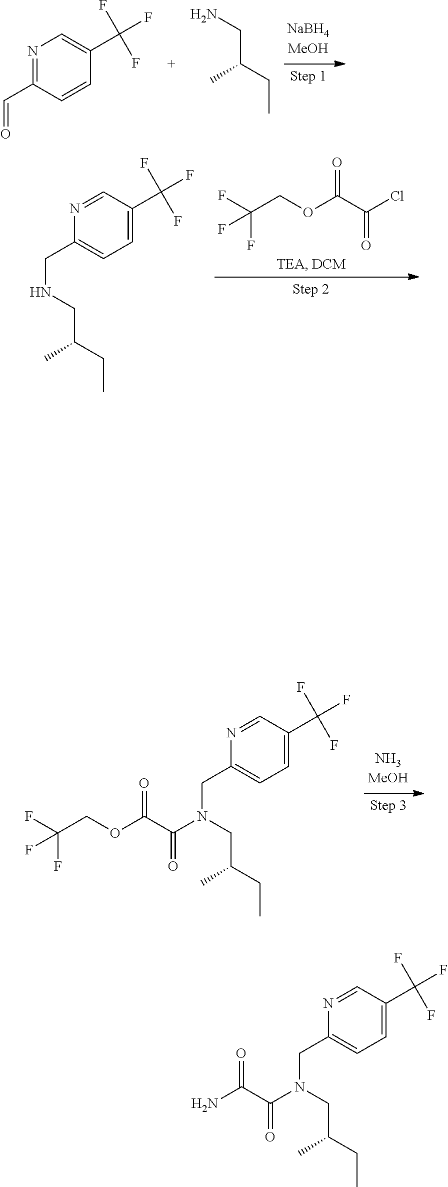

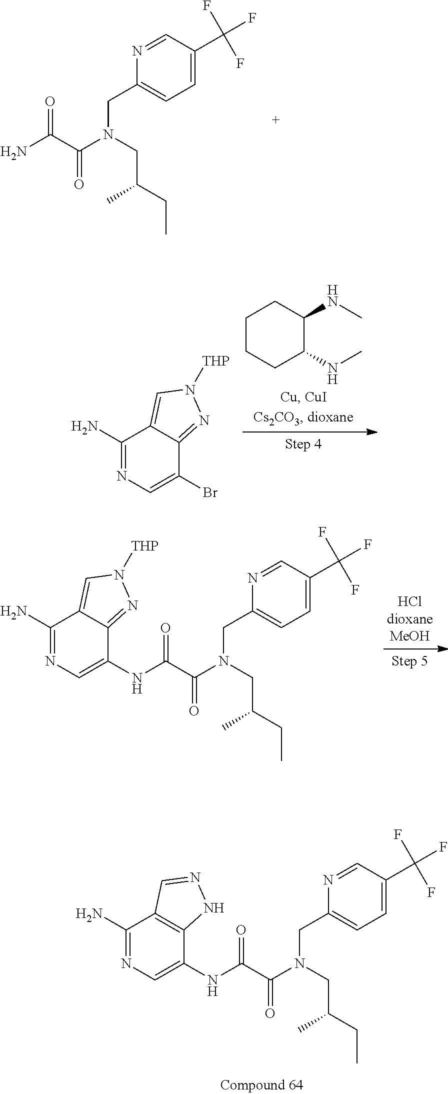

### Step 1: (S)-2-methyl-N-((5-(trifluoromethyl)pyridin-2-yl)methyl) butan-1-amine

(S)-2-methylbutan-1-amine (199.11 mg, 2.28 mmol) were added to the solution of 5-(trifluoromethyl)pyridine-2-carbaldehyde (0.4 g, 2.28 mmol) in Methanol (7.84 mL). The resulting mixture was stirred at 60° C. for 1 hour before Sodium Borohydride (172.83 mg, 4.57 mmol, 160.92 μL) was added portions thereto. After that, stirring was continued for 15 hr. Then, solvent was removed under reduced pressure and residue was partitioned between 10% aq. K₂CO₃ solution (20 mL) and DCM (20 mL). Organic layer was separated, dried over anhydrous sodium sulfate and concentrated under reduced pressure to afford(S)-2-methyl-N-((5-(trifluoromethyl)pyridin-2-yl)methyl) butan-1-amine (510 mg, crude) as a yellow oil.

LCMS (ESI): [M+H]⁺m/z: calcd 247.14; found 247.2; Rt=0.930 min.

### Step 2: (S)-2,2,2-trifluoroethyl 2-((2-methylbutyl)((5-(trifluoromethyl)pyridin-2-yl)methyl)amino)-2-oxoacetate

(S)-2-methyl-N-((5-(trifluoromethyl)pyridin-2-yl)methyl) butan-1-amine (510 mg, 2.07 mmol) and Triethylamine (314.33 mg, 3.11 mmol, 432.96 μL) were dissolved in DCM (10 mL) and the resulting mixture was cooled to −5° C. A solution of 2,2,2-trifluoroethyl 2-chloro-2-oxo-acetate (414.24 mg, 2.17 mmol) in DCM (2 mL) was added dropwise at −5° C. and the resulting mixture was allowed to warm to room temperature, and stirred overnight. Water (20 mL) was added and an organic layer was separated. The aqueous layer was extracted with DCM (20 mL) and combined organic layers were dried over anhydrous sodium sulfate, filtered, and concentrated in vacuo to obtain(S)-2,2,2-trifluoroethyl 2-((2-methylbutyl)((5-(trifluoromethyl)pyridin-2-yl)methyl)amino)-2-oxoacetate (735 mg, crude) as a light-yellow oil.

LCMS (ESI): [M+H]⁺m/z: calcd 401.13; found 401.2; Rt=1.407 min.

### Step 3: (S)-N1-(2-methylbutyl)-N1-((5-(trifluoromethyl)pyridin-2-yl)methyl)oxalamide

A solution of(S)-2,2,2-trifluoroethyl 2-((2-methylbutyl)((5-(trifluoromethyl)pyridin-2-yl)methyl)amino)-2-oxoacetate (735 mg, 1.84 mmol) in Methanol/NH3 (20 mL) was stirred at 25° C. for 18 hr. The solvent was evaporated to obtain(S)-N1-(2-methylbutyl)-N1-((5-(trifluoromethyl)pyridin-2-yl)methyl)oxalamide (565 mg, crude) as a yellow gum.

LCMS (ESI): [M+H]⁺m/z: calcd 318.15; found 318.2; Rt=1.288 min.

### Step 4: N1-(4-amino-2-(tetrahydro-2H-pyran-2-yl)-2H-pyrazolo[4,3-c]pyridin-7-yl)-N2-((S)-2-methylbutyl)-N2-((5-(trifluoromethyl)pyridin-2-yl)methyl)oxalamide

To an 8 mL vial(S)-N1-(2-methylbutyl)-N1-((5-(trifluoromethyl)pyridin-2-yl)methyl)oxalamide (0.15 g, 472.73 μmol), 7-bromo-2-tetrahydropyran-2-yl-pyrazolo[4,3-c]pyridin-4-amine (140.47 mg, 472.73 μmol), Copper (1.50 mg, 23.64 μmol), Copper (I) iodide (45.02 mg, 236.36 μmol, 8.01 μL), rac-(1R,2R)—N1,N2-dimethylcyclohexane-1,2-diamine (50.43 mg, 354.55 μmol), Cesium carbonate (308.05 mg, 945.46 μmol) and Dioxane (8.01 mL) were charged and the resulting mixture was spurred with argon for 5 min. The vial was sealed and heated at 100° C. for 48 hr. The reaction mixture was cooled, diluted with MeOH (5 mL), filtered and evaporated to obtain N1-(4-amino-2-(tetrahydro-2H-pyran-2-yl)-2H-pyrazolo[4,3-c]pyridin-7-yl)-N2-((S)-2-methylbutyl)-N2-((5-(trifluoromethyl)pyridin-2-yl)methyl)oxalamide (450 mg, crude) as a brown gum which was used in the next step without further purification.

LCMS (ESI): [M+H]⁺m/z: calcd 534.25; found 534.2; Rt=1.220 min.

### Step 5: The synthesis of(S)-N1-(4-amino-1H-pyrazolo[4,3-c]pyridin-7-yl)-N2-(2-methylbutyl)-N2-((5-(trifluoromethyl)pyridin-2-yl)methyl)oxalamide

N1-(4-amino-2-(tetrahydro-2H-pyran-2-yl)-2H-pyrazolo[4,3-c]pyridin-7-yl)-N2-((S)-2-methylbutyl)-N2-((5-(trifluoromethyl)pyridin-2-yl)methyl)oxalamide (450 mg, 844.98 μmol) was dissolved in Methanol (2 mL) and Dioxane/HCl (2 mL). The resulting mixture was stirred overnight. The reaction mixture was concentrated in vacuo. The residue was purified by HPLC (40-70% 2-10 min H₂O/ACN/0.1NH4OH flow 30 mL/min ((loading pump 4 mL ACN); target mass 450; column: Chromatorex C18 SMB100-5T 100×19 mm, 5 microM) and then repurified by HPLC (20-55% 2-10 min H₂O/MeOH/0.1FA flow 30 mL/min ((loading pump 4 mL MeOH); target mass 450; column: Chromatorex C18 SMB100-5T 100×19 mm, 5 microM) to obtain(S)-N1-(4-amino-1H-pyrazolo[4,3-c]pyridin-7-yl)-N2-(2-methylbutyl)-N2-((5-(trifluoromethyl)pyridin-2-yl)methyl)oxalamide (12.1 mg, 24.47 μmol, 2.90% yield, HCOOH) as a light-yellow solid.

¹H NMR (600 MHz, DMSO-d₆) δ 0.71-0.92 (m, 6H), 0.96-1.16 (m, 1H), 1.30-1.45 (m, 1H), 1.72-1.87 (m, 1H), 3.22 (dd, 1H), 3.47-3.61 (m, 1H), 4.71-4.88 (m, 1H), 5.02-5.12 (m, 1H), 6.55-7.00 (m, 2H), 7.42-7.74 (m, 2H), 8.15-8.30 (m, 2H), 8.69-8.97 (m, 1H), 10.25-10.51 (m, 1H), 12.72 (s, 1H).

LCMS (ESI): [M+H]⁺m/z: calcd 450.21; found 450.0; Rt=0.651 min.

### Example 55. Synthesis of (R)—N¹-(4-amino-1H-pyrazolo[4,3-c]pyridin-7-yl)-N2-(2-methylbenzyl)-N2-(2-methylbutyl)oxalamide (Compound 62)

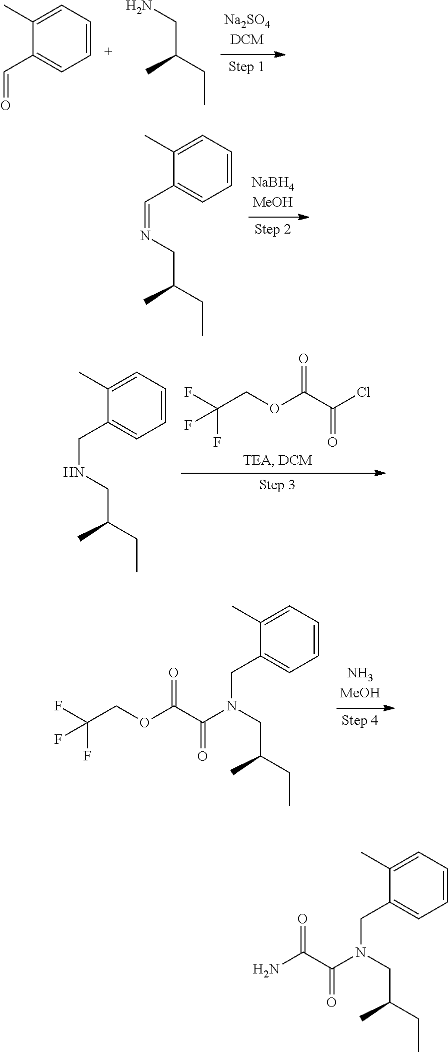

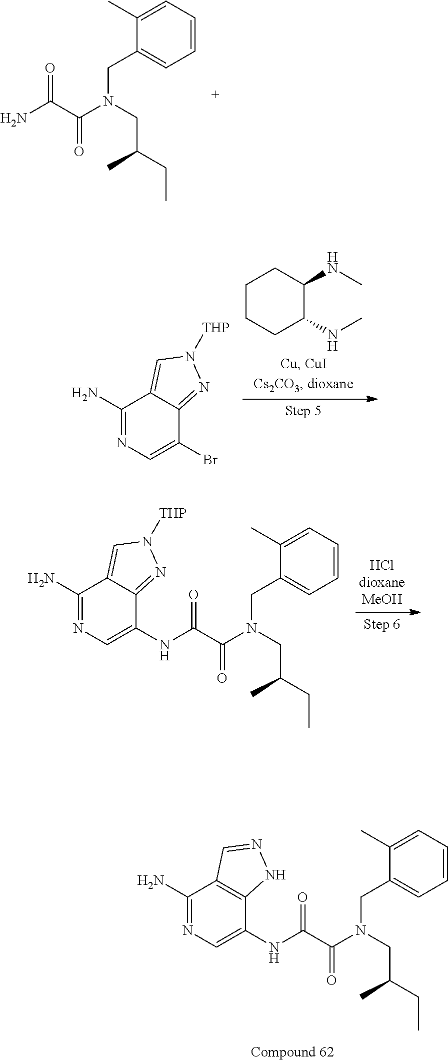

### Step 1: (R,Z)-2-methyl-N-(2-methylbenzylidene) butan-1-amine

2-methylbenzaldehyde (413.53 mg, 3.44 mmol, 398.01 μL) and (R)-2-methylbutan-1-amine (0.3 g, 3.44 mmol) was dissolved in DCM (25 mL), then Sodium sulfate, anhydrous (4.89 g, 34.42 mmol, 1.82 mL) was added and the reaction mixture was stirred overnight at 25° C. The reaction mixture was filtered, solid was washed with DCM and filtrate was concentrated on vacuo to give (R,Z)-2-methyl-N-(2-methylbenzylidene) butan-1-amine (655 mg, crude) as a yellow oil which was used in the next step without further purification.

### Step 2: (R)-2-methyl-N-(2-methylbenzyl) butan-1-amine

(R,Z)-2-methyl-N-(2-methylbenzylidene) butan-1-amine (655 mg, 3.46 mmol) was dissolved in Methanol (13 mL) and Sodium Borohydride (130.91 mg, 3.46 mmol, 121.89 μL) was added portionwise. After the addition was completed, the reaction mixture was stirred for 12 hr. Water (5 mL) was added to the reaction mixture and the resulting mixture was concentrated in vacuo. Water (20 mL) was added to the residue and the resulting mixture was extracted with DCM (2×25 mL). Combined organic layers were dried over anhydrous sodium sulfate, filtered, and concentrated in vacuo to obtain (R)-2-methyl-N-(2-methylbenzyl) butan-1-amine (280 mg, crude) as a colorless oil.

LCMS (ESI): [M+H]⁺m/z: calcd 192.18; found 192.2; Rt=0.889 min.

### Step 3: (R)-2,2,2-trifluoroethyl 2-((2-methylbenzyl)(2-methylbutyl)amino)-2-oxoacetate

(R)-2-methyl-N-(2-methylbenzyl) butan-1-amine (280 mg, 1.46 mmol) and Triethylamine (177.72 mg, 1.76 mmol, 244.79 μL) were dissolved in DCM (9.83 mL) and the resulting mixture was cooled to 25° C. in an ice/methanol bath. A solution of 2,2,2-trifluoroethyl 2-chloro-2-oxo-acetate (292.76 mg, 1.54 mmol, 199.16 μL) in DCM (1.97 mL) was added dropwise at 25° C. After the addition was completed, the resulting mixture was allowed to warm to room temperature and stirred overnight. Water (15 mL) was added to the reaction mixture and an organic layer was separated. The aqueous layer was extracted with DCM (25 mL) and combined organic layers were dried over anhydrous sodium sulfate, filtered and concentrated in vacuo to obtain (R)-2,2,2-trifluoroethyl 2-((2-methylbenzyl)(2-methylbutyl)amino)-2-oxoacetate (420 mg, crude) as a yellow oil.

LCMS (ESI): [M+H]⁺m/z: calcd 346.17; found 346.2; Rt=1.654 min.

### Step 4: (R)—N¹-(2-methylbenzyl)-N1-(2-methylbutyl)oxalamide

(R)-2,2,2-trifluoroethyl 2-((2-methylbenzyl)(2-methylbutyl)amino)-2-oxoacetate (420 mg, 1.22 mmol) was dissolved in Methanol/NH3 (7N)(25 mL) and the resulting solution was stirred overnight. The reaction mixture was concentrated in vacuo to obtain (R)—N¹-(2-methylbenzyl)-N1-(2-methylbutyl)oxalamide (327 mg, crude) as a yellow gum.

LCMS (ESI): [M+H]⁺m/z: calcd 263.18; found 263.2; Rt=1.150 min.

### Step 5: N1-(4-amino-2-(tetrahydro-2H-pyran-2-yl)-2H-pyrazolo[4,3-c]pyridin-7-yl)-N2-(2-methylbenzyl)-N2-((R)-2-methylbutyl)oxalamide

To an 8 ml vial (R)—N¹-(2-methylbenzyl)-N1-(2-methylbutyl)oxalamide (150 mg, 571.76 μmol), 7-bromo-2-tetrahydropyran-2-yl-pyrazolo[4,3-c]pyridin-4-amine (186.89 mg, 628.94 μmol), Copper (1.82 mg, 28.59 μmol), Copper (I) iodide (54.45 mg, 285.88 μmol, 9.69 μL), rac-(1R,2R)—N1,N2-dimethylcyclohexane-1,2-diamine (61.00 mg, 428.82 μmol), Cesium carbonate (372.58 mg, 1.14 mmol) and Dioxane (3 mL) were charged and the resulting mixture was splurged with argon for 5 min. The vial was sealed and heated at 110° C. for 38 hr. The reaction mixture was cooled, diluted with MeOH (5 mL), and filtered. The filtrate was concentrated in vacuo. The residue was purified by HPLC (0-2-10 min 43-50-70% H₂O/MeOH/0.1NH4OH, flow 30 mL/min (loading pump 4 mL MeOH, target mass 465) to obtain N1-(4-amino-2-(tetrahydro-2H-pyran-2-yl)-2H-pyrazolo[4,3-c]pyridin-7-yl)-N2-(2-methylbenzyl)-N2-((R)-2-methylbutyl)oxalamide (29.2 mg, crude) as a yellow gum.

LCMS (ESI): [M+H]⁺m/z: calcd 479.32; found 479.2; Rt=1.278 min.

### Step 6: The synthesis of (R)—N¹-(4-amino-1H-pyrazolo[4,3-c]pyridin-7-yl)-N2-(2-methylbenzyl)-N2-(2-methylbutyl)oxalamide

N1-(4-amino-2-(tetrahydro-2H-pyran-2-yl)-2H-pyrazolo[4,3-c]pyridin-7-yl)-N2-(2-methylbenzyl)-N2-((R)-2-methylbutyl)oxalamide (29.2 mg, 61.01 μmol) was dissolved in Methanol (1 mL) and HCl/Dioxane (1 mL) was added thereto. The resulting solution was stirred for 1 hr and the reaction mixture was concentrated in vacuo. The residue was purified by HPLC (0-2-10 min 0-85% H₂O/ACN/0.1FA flow 30 mL/min (loading pump 4 mL ACN), target mass 395; COLUMN: Chromatorex C18 SMB100-5T 100×19 mm, 5 microM) to obtain (R)—N¹-(4-amino-1H-pyrazolo[4,3-c]pyridin-7-yl)-N2-(2-methylbenzyl)-N2-(2-methylbutyl)oxalamide (8.5 mg, 21.55 μmol, 35.32% yield) as a light-yellow gum.

¹H NMR (600 MHz, DMSO-d₆) δ 0.71-0.92 (m, 6H), 0.96-1.16 (m, 1H), 1.30-1.45 (m, 1H), 1.72-1.87 (m, 1H), 3.22 (dd, 1H), 3.47-3.61 (m, 1H), 4.71-4.88 (m, 1H), 5.02-5.12 (m, 1H), 6.55-7.00 (m, 2H), 7.42-7.74 (m, 2H), 8.15-8.30 (m, 2H), 8.69-8.97 (m, 1H), 10.25-10.51 (m, 1H), 12.72 (s, 1H).

LCMS (ESI): [M+H]⁺m/z: calcd 395.25; found 395.4; Rt=1.595 min.

### Example 56. Synthesis of(S)-N1-(4-amino-1H-pyrazolo[4,3-c]pyridin-7-yl)-N2-(2-methylbenzyl)-N2-(2-methylbutyl)oxalamide (Compound 60)

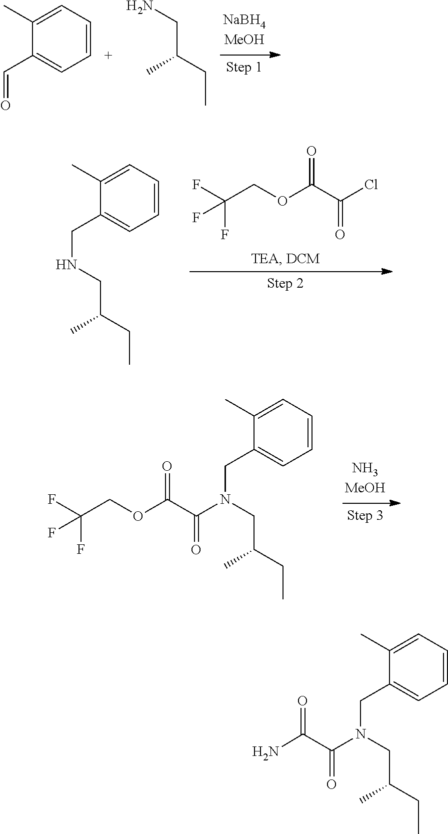

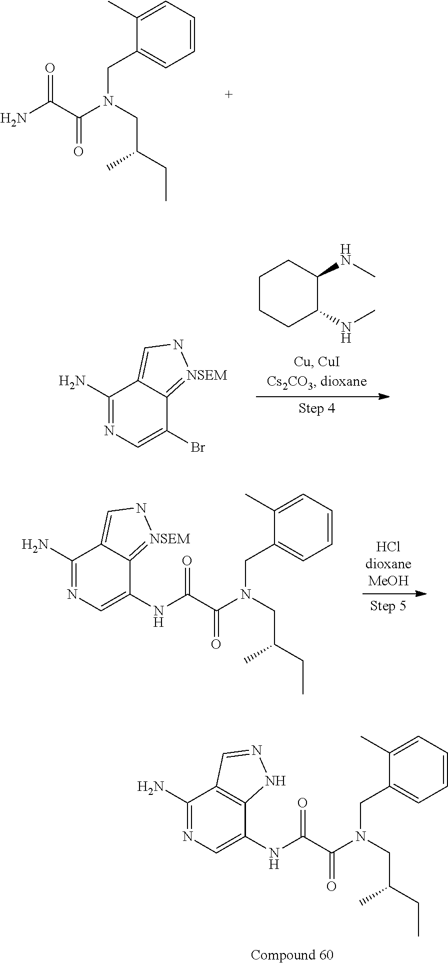

### Step 1: (S)-2-methyl-N-(2-methylbenzyl) butan-1-amine

(S)-2-methylbutan-1-amine (114.62 mg, 1.32 mmol) were added to the solution of 2-methylbenzaldehyde (0.158 g, 1.32 mmol, 152.07 μL) in MeOH (5 mL). The resulting mixture was stirred at 60° C. for 1 hour before. Sodium Borohydride (99.50 mg, 2.63 mmol, 92.64 μL) was added portions thereto. After that, stirring was continued for 16 hr. Then, solvent was removed under reduced pressure and residue was partitioned between 10% aq. K₂CO₃ solution (20 mL) and DCM (20 mL). Organic layer was separated, dried over anhydrous sodium sulfate and concentrated under reduced pressure, leaving(S)-2-methyl-N-(2-methylbenzyl) butan-1-amine (0.2 g, 1.05 mmol, 79.50% yield) as a light-yellow oil.

LCMS (ESI): [M+H]⁺m/z: calcd 192.18; found 192.2; Rt=0.770 min.

### Step 2: (S)-2,2,2-trifluoroethyl 2-((2-methylbenzyl)(2-methylbutyl)amino)-2-oxoacetate

2,2,2-trifluoroethyl 2-chloro-2-oxo-acetate (219.07 mg, 1.15 mmol) was added dropwise to a solution of(S)-2-methyl-N-(2-methylbenzyl) butan-1-amine (0.2 g, 1.05 mmol) and triethylamine (126.94 mg, 1.25 mmol, 174.85 μL) in DCM (5 mL) at 0° C. After addition was complete, cooling bath was removed and resulting mixture was allowed to warm up to 20° C. and stirred for 16 hr. Then, it was washed with water, dried over anhydrous sodium sulfate and concentrated under reduced pressure, affording(S)-2,2,2-trifluoroethyl 2-((2-methylbenzyl)(2-methylbutyl)amino)-2-oxoacetate (324 mg, 938.16 μmol, 89.74% yield) as a yellow oil which was used in the next step without further purification.

LCMS (ESI): [M+H]⁺m/z: calcd 346.17; found 346.0; Rt=1.366 min.

### Step 3: (S)-N1-(2-methylbenzyl)-N1-(2-methylbutyl)oxalamide

A solution of(S)-2,2,2-trifluoroethyl 2-((2-methylbenzyl)(2-methylbutyl)amino)-2-oxoacetate (324 mg, 938.16 μmol) in Methanol/NH3 (5N)(10 mL) was stirred at 20° C. for 14 hr. The solvent was evaporated to obtain(S)-N1-(2-methylbenzyl)-N1-(2-methylbutyl)oxalamide (0.25 g, crude) as a yellow solid.

### Step 4: (S)-N1-(4-amino-1-((2-(trimethylsilyl) ethoxy)methyl)-1H-pyrazolo[4,3-c]pyridin-7-yl)-N2-(2-methylbenzyl)-N2-(2-methylbutyl)oxalamide

(S)-N1-(2-methylbenzyl)-N1-(2-methylbutyl)oxalamide (0.11 g, 419.29 μmol), 7-bromo-1-(2-trimethylsilylethoxymethyl)pyrazolo[4,3-c]pyridin-4-amine (158.33 mg, 461.22 μmol), Copper (I) iodide (15.97 mg, 83.86 μmol, 2.84 μL), Cesium carbonate (273.23 mg, 838.59 μmol) and (1R,2R)—N1,N2-dimethylcyclohexane-1,2-diamine (71.57 mg, 503.15 μmol) were mixed in dioxane (5 mL) under argon, and then stirred overnight at 100° C. for 36 hr in vial. The reaction mixture was filtered and the filtrate was concentrated in vacuo and the residue was purified by HPLC (SYSTEM 0-2-10 min 43-50-80% MeOH/H₂O+FA 30 mL/min (loading pump 4 mL MeOH), target MI 525 column: Chromatorex C18 SMB100-5T 100×19, 5microM) to give(S)-N1-(4-amino-1-((2-(trimethylsilyl) ethoxy)methyl)-1H-pyrazolo[4,3-c]pyridin-7-yl)-N2-(2-methylbenzyl)-N2-(2-methylbutyl)oxalamide (33.5 mg, 63.84 μmol, 15.23% yield) as a brown gum.

LCMS (ESI): [M+H]⁺m/z: calcd 525.36; found 525.2; Rt=1.503 min.

### Step 5: The synthesis of(S)-N1-(4-amino-1H-pyrazolo[4,3-c]pyridin-7-yl)-N2-(2-methylbenzyl)-N2-(2-methylbutyl)oxalamide

To a solution of(S)-N1-(4-amino-1-((2-(trimethylsilyl) ethoxy)methyl)-1H-pyrazolo[4,3-c]pyridin-7-yl)-N2-(2-methylbenzyl)-N2-(2-methylbutyl)oxalamide (33.5 mg, 63.84 μmol) in MeOH (3 mL) was added Hydrogen chloride solution 4.0M in dioxane (800.00 mg, 21.94 mmol, 1 mL) at 20° C. The resulting mixture was left to stirred for 14 hr. The resulting mixture was evaporated to dryness and then submitted to reverse phase HPLC (SYSTEM 0-2-10 minMar. 10, 1955% H₂O/ACN/0.1FA, flow 30 mL/min (loading pump 4 mL ACN), target mass 394 column: Chromatorex C18 100×19 mm, 5 microM) to afford(S)-N1-(4-amino-1H-pyrazolo[4,3-c]pyridin-7-yl)-N2-(2-methylbenzyl)-N2-(2-methylbutyl)oxalamide (18.3 mg, 41.54 μmol, 65.07% yield, HCOOH) as a yellow gum.

¹H NMR (600 MHz, DMSO-d₆) δ 0.71-0.81 (m, 3H), 0.81-0.86 (m, 3H), 0.96-1.12 (m, 1H), 1.21-1.45 (m, 1H), 1.66-1.81 (m, 1H), 2.23-2.31 (m, 3H), 3.09-3.15 (m, 1H), 4.51-4.70 (m, 1H), 4.70-4.89 (m, 1H), 6.59-6.69 (m, 2H), 7.13-7.16 (m, 1H), 7.17-7.23 (m, 3H), 7.49-7.81 (m, 1H), 8.12-8.22 (m, 2H), 10.33-10.51 (m, 1H), 12.75 (br s, 1H).

LCMS (ESI): [M+H]⁺m/z: calcd 395.25; found 395.4; Rt=1.132 min.

### Example 57. Synthesis of N-(4-Amino-1H-pyrazolo[4,3-c]pyridin-7-yl)-N′-methyl-N′—[(1R)-1-[2-fluoro-4-(trifluoromethyl)phenyl]ethyl]oxamide (Compound 54) and N-(4-Amino-1H-pyrazolo[4,3-c]pyridin-7-yl)-N′-methyl-N′—[(1S)-1-[2-fluoro-4-(trifluoromethyl)phenyl]ethyl]oxamide (Compound 74)

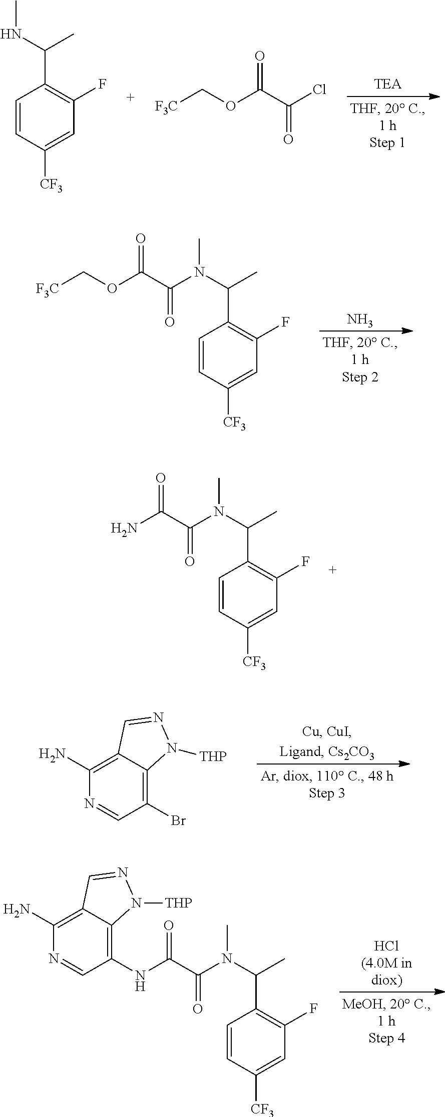

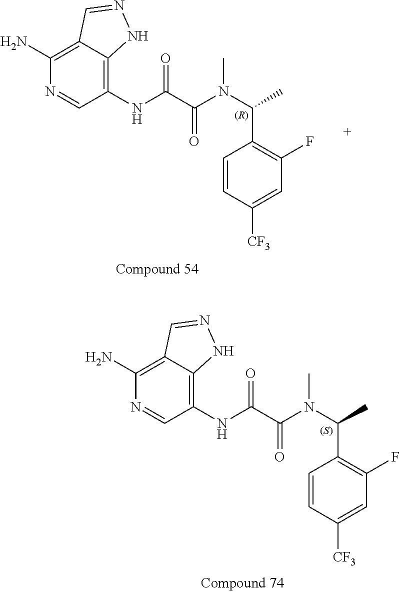

### Step 1: The Synthesis of 2,2,2-Trifluoroethyl 2-[1-[2-fluoro-4-(trifluoromethyl)phenyl]ethyl-methyl-amino]-2-oxo-acetate

2,2,2-Trifluoroethyl 2-chloro-2-oxo-acetate (378.95 mg, 1.99 mmol) was added dropwise to a stirred solution of 1-[2-fluoro-4-(trifluoromethyl)phenyl]-N-methyl-ethanamine (0.4 g, 1.81 mmol) and TEA (219.59 mg, 2.17 mmol, 302.46 μL) in THF (19.82 mL) at 20° C., stirred for 1 hr at 20° C. Reaction mixture was used in the next step.

LCMS (ESI): [M+H]⁺m/z: calcd 376.2; found 376.2; Rt=1.301 min.

### Step 2: The Synthesis of N′-[1-[2-Fluoro-4-(trifluoromethyl)phenyl]ethyl]-N′-methyl-oxamide

Ammonia (599.09 mg, 35.18 mmol) was bubbled through a reaction mixture from previous step at 20° C., stirred for 1 hr at 20° C. Reaction mixture was filtered, solid washed with THF (2×5 mL), filtrate concentrated in vacuo to give pure N′-[1-[2-fluoro-4-(trifluoromethyl)phenyl]ethyl]-N′-methyl-oxamide (0.35 g, 1.20 mmol, 68.09% yield).

### Step 3: The Synthesis of N-(4-Amino-2-tetrahydropyran-2-yl-pyrazolo[4,3-c]pyridin-7-yl)-N′-[1-[2-fluoro-4-(trifluoromethyl)phenyl]ethyl]-N′-methyl-oxamide

Copper (1.26 mg, 19.77 μmol), Copper (I) iodide (37.65 mg, 197.70 μmol, 6.70 μL), cesium carbonate (193.24 mg, 593.09 μmol) was added to a stirred solution of N′-[1-[2-fluoro-4-(trifluoromethyl)phenyl]ethyl]-N′-methyl-oxamide (115.55 mg, 395.40 μmol), 7-bromo-2-tetrahydropyran-2-yl-pyrazolo[4,3-c]pyridin-4-amine (117.49 mg, 395.40 μmol), rac-(1R,2R)—N1,N2-dimethylcyclohexane-1,2-diamine (28.12 mg, 197.70 μmol) in 1,4-dioxane (7.00 mL) under Ar atmosphere and stirred at 110° C. for 48 hr in closed vial. RM was cooled to r.t., filtered, solid washed with dioxane (2×3 mL), filtrate used in the next step.

LCMS (ESI): [M+H]⁺m/z: calcd 509.2; found 509.2; Rt=1.046 min.

### Step 4: The Synthesis of N-(4-Amino-1H-pyrazolo[4,3-c]pyridin-7-yl)-N′-methyl-N′—[(1R)-1-[2-fluoro-4-(trifluoromethyl)phenyl]ethyl]oxamide (Compound 54) and N-(4-Amino-1H-pyrazolo[4,3-c]pyridin-7-yl)-N′-methyl-N′—[(1S)-1-[2-fluoro-4-(trifluoromethyl)phenyl]ethyl]oxamide (Compound 74)

Hydrogen chloride solution 4.0M in dioxane (700.53 mg, 19.21 mmol, 875.66 μL) was added to a solution of N-(4-amino-2-tetrahydropyran-2-yl-pyrazolo[4,3-c]pyridin-7-yl)-N′-[1-[2-fluoro-4-(trifluoromethyl)phenyl]ethyl]-N′-methyl-oxamide (195.39 mg, 384.27 μmol) in Methanol (2 mL) and stirred at 20° C. for 1 hr. Volatiles was evaporated in vacuo, the residue triturated with IPA (5 mL), filtered, washed with IPA (5 mL), and submitted to HPLC (column: Chromatorex 18 SMB 100-5T 100×19 mm 5 μm; Oct. 10, 2030% 0-1.3-5.3 min H₂O/ACN/0.1% FA, flow rate: 30 mL/min) to give 16 mg of racemic product. Enantiomers was separated on preparative chiral HPLC (Column: CHIRALPAK IC (250×30 mm, 10 mkm); Mobile Phase: Hexane: IPA: MeOH: DEA, 70:15:15:0.2; Injection Volume: 900 mkL) to give N-(4-amino-1H-pyrazolo[4,3-c]pyridin-7-yl)-N′-methyl-N′—[(1R)-1-[2-fluoro-4-(trifluoromethyl)phenyl]ethyl]oxamide (6.7 mg, 15.79 μmol, 4.11% yield; RT (F1)=10.10 min) and N-(4-amino-1H-pyrazolo[4,3-c]pyridin-7-yl)-N′-methyl-N′—[(1S)-1-[2-fluoro-4-(trifluoromethyl)phenyl]ethyl]oxamide (8.5 mg, 20.03 μmol, 5.21% yield; RT (F2)=13.39 min).

### Compound 54:

Preparative: RT (Column: CHIRALPAK IC (250×30 mm, 10 mkm); Mobile Phase: Hexane: IPA: MeOH: DEA, 70:15:15:0.2; Flow Rate: 40 mL/min)=14.844 min.

Analytical: RT (Column: Chiralpak IC (250×4.6 mm, 5 mkm)-1; Mobile Phase: Hexane (0.1% EDA): IPA: MeOH, 50:25:25; Flow Rate: 0.6 mL/min)=10.104 min.

LCMS (ESI): [M+H]⁺m/z: calcd 425.2; found 425.2; Rt=2.641 min.

### Compound 74:

Preparative: RT (Column: CHIRALPAK IC (250×30 mm, 10 mkm); Mobile Phase: Hexane: IPA: MeOH: DEA, 70:15:15:0.2; Flow Rate: 40 mL/min)=22.571 min.

Analytical: RT (Column: Chiralpak IC (250×4.6 mm, 5 mkm)-1; Mobile Phase: Hexane (0.1% EDA): IPA: MeOH, 50:25:25; Flow Rate: 0.6 mL/min)=13.391 min.

LCMS (ESI): [M+H]⁺m/z: calcd 425.2; found 425.2; Rt=2.641 min.

The absolute stereochemistry of the two compound was independently confirmed.

### Example 58. Synthesis of N-(4-amino-1H-pyrazolo[4,3-c]pyridin-7-yl)-N′-(o-tolylmethyl)-N′-[5-(trifluoromethyl)-2-pyridyl]methyl]oxamide (Compound 76)

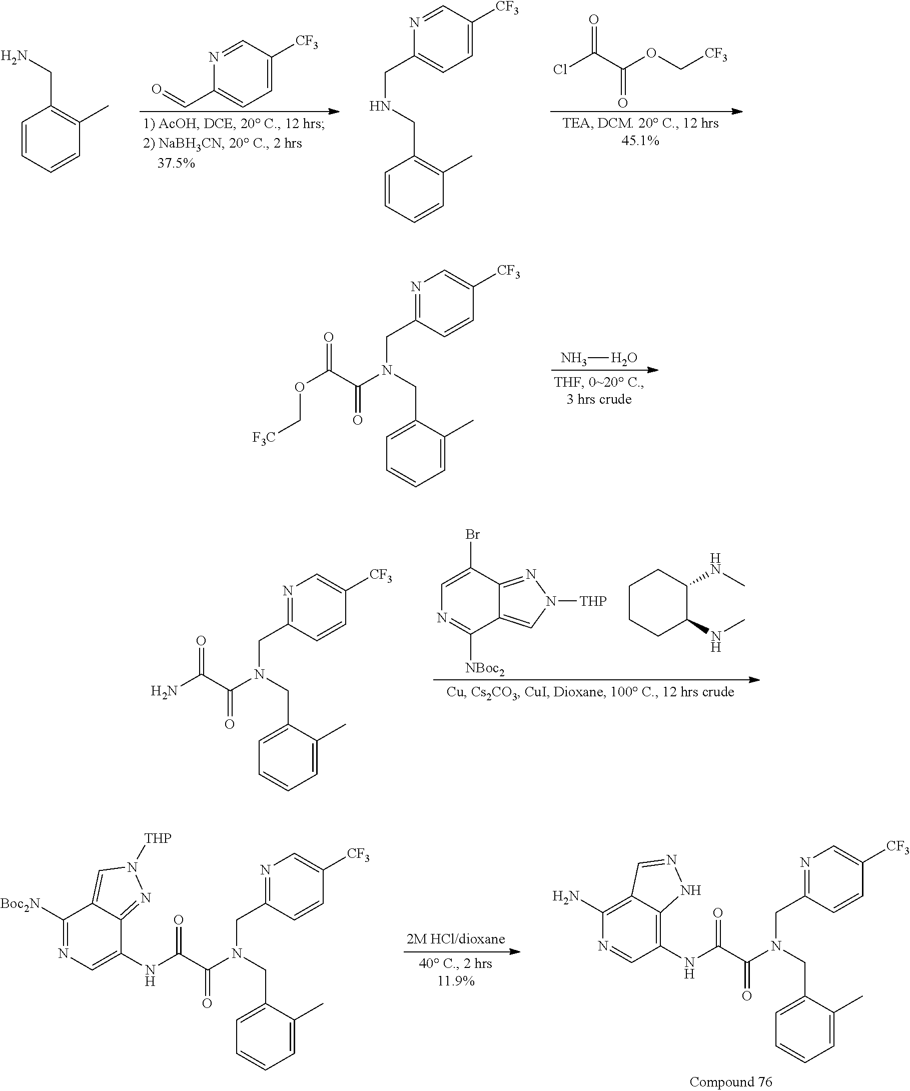

### Step 1: Synthesis of 1-(o-tolyl)-N-[[5-(trifluoromethyl)-2-pyridyl]methyl]methanamine

To a mixture of o-tolylmethanamine (0.8 mL, 6.85 mmol), 5-(trifluoromethyl)pyridine-2-carbaldehyde (1 g, 5.71 mmol) in DCE (10 mL) was added AcOH (0.3 mL, 5.23 mmol). The resulting mixture was stirred at 20° C. for 12 hours. The resulting mixture was added NaBH₃CN (538 mg, 8.56 mmol) and then stirred at 20° C. for 2 hours. The resulting mixture was extracted with H₂O(50 mL) and DCM (50 mL×2). The combined organic layer was dried over anhydrous Na₂SO₄, filtered and concentrated under reduced pressure. The residue was purified by flash chromatography (ISCO®; 12 g SepaFlash® Silica Flash Column, Petroleum Ether/EtOAc with EtOAc from 0~30%, flow rate=20 mL/min, 254 nm). The residue was purified by preparative TLC (silica, petroleum ether/EtOAc=1:1) to afford 1-(o-tolyl)-N-[5-(trifluoromethyl)-2-pyridyl]methyl]methanamine (600 mg, crude) as yellow oil. LCMS (ESI) [M+H]⁺m/z: calcd 281.1, found 281.2.

### Step 2: Synthesis of 2,2,2-trifluoroethyl 2-[o-tolylmethyl-[[5-(trifluoromethyl)-2-pyridyl]methyl]amino]-2-oxo-acetate

To a mixture of 1-(o-tolyl)-N-[[5-(trifluoromethyl)-2-pyridyl]methyl]methanamine (500 mg, 1.78 mmol), TEA (0.3 mL, 2.67 mmol) in DCM (5 mL) at 0° C. was added 2,2,2-trifluoroethyl 2-chloro-2-oxo-acetate (679 mg, 3.56 mmol) and then the resulting mixture stirred at 20° C. for 12 hours. The resulting mixture concentrated under reduced pressure. The residue was purified by flash chromatography (ISCO®; 12 g SepaFlash® Silica Flash Column, Petroleum Ether/EtOAc with EtOAc from 0~30%, flow rate=20 mL/min, 254 nm) to afford 2,2,2-trifluoroethyl 2-[o-tolylmethyl-[[5-(trifluoromethyl)-2-pyridyl]methyl]amino]-2-oxo-acetate (350 mg, 45.2% yield) as yellow liquid. ¹H NMR (400 MHz, DMSO-d₆) δ ppm 8.91 (s, 1 H), 8.16-8.20 (m, 1H), 7.47 (d, J=8.4 Hz, 1H), 7.04-7.21 (m, 4H), 4.90-5.04 (m, 2H), 4.63-4.73 (m, 4H), 2.18 (s, 3H); LCMS (ESI) [M+H]⁺m/z: calcd 435.1, found 435.2.

### Step 3: Synthesis of N′-(o-tolylmethyl)-N′-[[5-(trifluoromethyl)-2-pyridyl]methyl]oxamide

To a solution of 2,2,2-trifluoroethyl 2-[o-tolylmethyl-[5-(trifluoromethyl)-2-pyridyl]methyl]amino]-2-oxo-acetate (350 mg, 0.805 mmol) in THF (5 mL) was added NH3H₂O (282 mg, 8.05 mmol) at 0° C. The mixture was stirred at 20° C. for 3 hours. The residue was diluted with NH₄Cl (50 mL) and extracted with EtOAc (50 mL×3). The combined organic layers were washed with brine (50 mL), dried over anhydrous Na₂SO₄, filtered and concentrated under reduced pressure to afford N′-(o-tolylmethyl)-N′-[[5-(trifluoromethyl)-2-pyridyl]methyl]oxamide (310 mg, crude) as yellow oil. ¹H NMR (400 MHz, DMSO-d₆) & ppm 8.86-8.90 (m, 1H), 8.14-8.30 (m, 2H), 7.79 (br d, J=10.0 Hz, 1H), 7.57 (d, J=8.4 Hz, 1H), 7.05-7.22 (m, 4H), 4.74 (d, J=4.4 Hz, 2H), 4.55 (s, 2H), 2.17 (s, 3H); LCMS (ESI) [M+H]⁺m/z: calcd 352.1, found 351.9.

### Step 4: Synthesis of tert-butyl N-tert-butoxycarbonyl-N-[7-[[2-[o-tolylmethyl-[[5-(trifluoromethyl)-2-pyridyl]methyl]amino]-2-oxo-acetyl]amino]-2-tetrahydropyran-2-yl-pyrazolo[4,3-c]pyridin-4-yl]carbamate

To a mixture of N′-(o-tolylmethyl)-N′-[5-(trifluoromethyl)-2-pyridyl]methyl]oxamide (200 mg, 0.569 mmol), tert-butyl N-(7-bromo-2-tetrahydropyran-2-yl-pyrazolo[4,3-c]pyridin-4-yl)-N-tert-butoxycarbonyl-carbamate (284 mg, 0.570 mmol), rac-(1S,2S)-N1,N2-dimethylcyclohexane-1,2-diamine (120 mg, 0.843 mmol) and Cs₂CO₃ (370 mg, 1.14 mmol) in dioxane (5 mL) was added Cu(36 mg, 0.566 mmol) and CuI (108 mg, 567 mmol), the resulting mixture stirred at 100° C. for 12 hours. The reaction mixture was filtrated and washed with EtOAc (50 mL). The residue (The combined organic layers) was diluted with NH3.H₂O(50 mL) and extracted with EtOAc (50 mL×3). The combined organic layers were washed with brine (50 mL), dried over anhydrous Na₂SO₄, filtered and concentrated under reduced pressure to afford tert-butyl N-tert-butoxycarbonyl-N-[7-[2-[o-tolylmethyl-[[5-(trifluoromethyl)-2-pyridyl]methyl]amino]-2-oxo-acetyl]amino]-2-tetrahydropyran-2-yl-pyrazolo[4,3-c]pyridin-4-yl]carbamate (490 mg, crude) as yellow oil. LCMS (ESI) [M+H]⁺m/z: calcd 768.3, found 768.2.

### Step 5: Synthesis of N-(4-amino-1H-pyrazolo[4,3-c]pyridin-7-yl)-N′-(o-tolylmethyl)-N′-[[5-(trifluoromethyl)-2-pyridyl]methyl]oxami (Compound 76)

To a solution of tert-butyl N-tert-butoxycarbonyl-N-[7-[2-[o-tolylmethyl-[5-(trifluoromethyl)-2-pyridyl]methyl]amino]-2-oxo-acetyl]amino]-2-tetrahydropyran-2-yl-pyrazolo[4,3-c]pyridin-4-yl]carbamate (200 mg, 260 mmol) was added 2M HCl/dioxane (0.1 mL, 0.2 mmol) and dioxane (4 mL). The mixture was stirred at 40° C. for 2 hours. The reaction mixture was adjusted to pH~8 with saturated NaHCO₃/H₂O. The resultant mixture was dried over Na₂SO₄ and filtered. The filtrate was concentrated under reduced pressure. The residue was purified by preparative HPLC (Instrument: AD; Column: 2_Phenomenex Gemini C18 75× 40 mm×3 μm; Mobile phase A: water (NH3H₂O+NH₄HCO₃). B: ACN; Gradient: B from 35% to 65% in 9.5 min, hold 100% B for 4 min; Flow Rate: 30 ml/min; Column Temperature: 30° C.; Wavelength: 220 nm, 254 nm) to afford N-(4-amino-1H-pyrazolo[4,3-c]pyridin-7-yl)-N′-(o-tolylmethyl)-N′-[5-(trifluoromethyl)-2-pyridyl]methyl]oxamide (15 mg, 11.9% yield, 99.7% purity) was obtained as white solid.

Compound 76: 1H NMR (400 MHz, DMSO-d₆) δ ppm 12.86 (br s, 1H), 10.55-10.62 (m, 1H), 8.88-8.96 (m, 1H), 8.15-8.24 (m, 2H), 7.59-7.66 (m, 2H), 7.15-7.23 (m, 4H), 6.78 (br s, 2H), 5.00 (br d, J=7.2 Hz, 2H), 4.66-4.70 (m, 2H), 2.20-2.25 (m, 3H); LCMS (ESI) [M+H]⁺m/z: calcd 484.2, found 484.1; HPLC: 97.14% @220 nm, 99.70% @254 nm.

### Example 59. Synthesis of N-(4-amino-1HI-pyrazolo[4,3-c]pyridin-7-yl)-N′-[(4-fluoro-2-methyl-phenyl)methyl]-N′-isobutyl-oxamide (Compound 71)

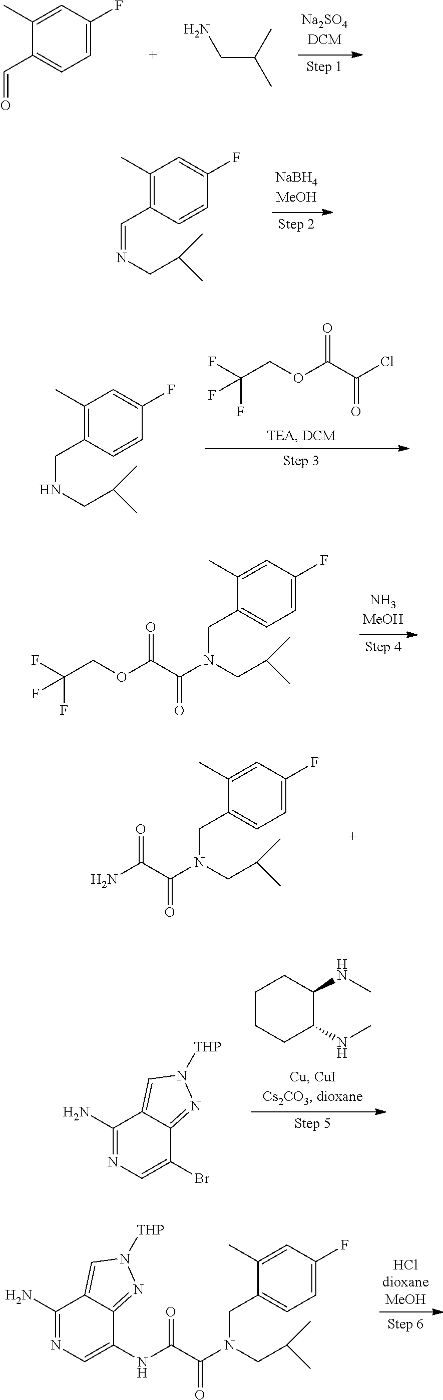

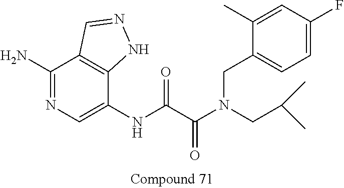 [SMILES: Cc1cc(F)ccc1CN(CC(C)C)C(=O)C(=O)Nc1cnc(N)c2cn[nH]c12]

### Step 1: (Z)-1-(4-fluoro-2-methyl-phenyl)-N-isobutyl-methanimine

4-fluoro-2-methyl-benzaldehyde (500 mg, 3.62 mmol, 437.06 μL) was dissolved in DCM (8 mL) and 2-methylpropan-1-amine (264.72 mg, 3.62 mmol, 359.68 μL) was added thereto followed by the addition of Sodium sulfate, anhydrous (2.06 g, 14.48 mmol, 767.36 μL). The resulting mixture was vigorously stirred overnight. The reaction mixture was filtered and concentrated in vacuo to obtain (Z)-1-(4-fluoro-2-methyl-phenyl)-N-isobutyl-methanimine (692 mg, 3.58 mmol, 98.93% yield) as a light-yellow liquid.

### Step 2: N-[(4-fluoro-2-methyl-phenyl)methyl]-2-methyl-propan-1-amine

(Z)-1-(4-fluoro-2-methyl-phenyl)-N-isobutyl-methanimine (692 mg, 3.58 mmol) was dissolved in MeOH (10 mL) and Sodium Borohydride (406.40 mg, 10.74 mmol, 378.40 μL) was added portionwise. After the addition was completed, the reaction mixture was stirred for 1 hr. Water (5 mL) was added to the reaction mixture and the resulting mixture was concentrated in vacuo. Water (20 mL) was added to the residue and the resulting mixture was extracted with DCM (2×25 mL). Combined organic layers were dried over anhydrous sodium sulfate, filtered, and concentrated in vacuo to obtain N-[(4-fluoro-2-methyl-phenyl)methyl]-2-methyl-propan-1-amine (687 mg, 3.52 mmol, 98.25% yield) as a colorless oil.

LCMS (ESI): [M+H]⁺m/z: calcd 196.15; found 196.2; Rt=0.712 min.

### Step 3:2,2,2-trifluoroethyl 2-[(4-fluoro-2-methyl-phenyl)methyl-isobutyl-amino]-2-oxo-acetate

N-[(4-fluoro-2-methyl-phenyl)methyl]-2-methyl-propan-1-amine (687 mg, 3.52 mmol) and Triethylamine (391.60 mg, 3.87 mmol, 539.39 μL) were dissolved in DCM (10 mL) and the resulting mixture was cooled to −5° C. in an ice/methanol bath. A solution of 2,2,2-trifluoroethyl 2-chloro-2-oxo-acetate (703.73 mg, 3.69 mmol) in DCM (3 mL) was added dropwise at −5° C. After the addition was completed, the resulting mixture was allowed to warm to room temperature and stirred overnight. Water (15 mL) was added to the reaction mixture and an organic layer was separated. The aqueous layer was extracted with DCM (25 mL) and combined organic layers were dried over anhydrous sodium sulfate, filtered and concentrated in vacuo to obtain 2,2,2-trifluoroethyl 2-[(4-fluoro-2-methyl-phenyl)methyl-isobutyl-amino]-2-oxo-acetate (1.02 g, 2.91 mmol, 82.75% yield) as a colorless gum.

LCMS (ESI): [M+H]⁺m/z: calcd 350.14; found 350.2; Rt=1.591 min.

### Step 4: N′-[(4-fluoro-2-methyl-phenyl)methyl]-N′-isobutyl-oxamide

2,2,2-trifluoroethyl 2-[(4-fluoro-2-methyl-phenyl)methyl-isobutyl-amino]-2-oxo-acetate (1.02 g, 2.92 mmol) was dissolved in MeOH (5 mL) and NH3/MeOH (20 mL) was added thereto. The resulting solution was stirred overnight. The reaction mixture was filtered and the filtrate was concentrated in vacuo to obtain N′-[(4-fluoro-2-methyl-phenyl)methyl]-N′-isobutyl-oxamide (785 mg, crude) as a yellow gum.

LCMS (ESI): [M+Na]⁺m/z: calcd 289.15; found 289.0; Rt=1.171 min.

### Step 5: N-(4-amino-2-tetrahydropyran-2-yl-pyrazolo[4,3-c]pyridin-7-yl)-N′-[(4-fluoro-2-methyl-phenyl)methyl]-N′-isobutyl-oxamide

To an 8 ml vial N′-[(4-fluoro-2-methyl-phenyl)methyl]-N′-isobutyl-oxamide (171 mg, 642.11 μmol), 7-bromo-2-tetrahydropyran-2-yl-pyrazolo[4,3-c]pyridin-4-amine (209.88 mg, 706.32 μmol), Copper (2.04 mg, 32.11 μmol), Copper (I) iodide (61.14 mg, 321.05 μmol, 10.88 μL), rac-(1R,2R)—N1,N2-dimethylcyclohexane-1,2-diamine (68.50 mg, 481.58 μmol), Cesium carbonate (418.42 mg, 1.28 mmol) and Dioxane (3.5 mL) were charged. The resulting mixture was splurged with argon for 5 min. The vial was sealed and heated at 110° C. for 65 hr. The reaction mixture was cooled, diluted with MeOH (5 mL), and filtered. The filtercake was rinsed with MeOH (5 mL) and the filtrate was concentrated in vacuo. The residue was submitted to HPLC and purified (0-2-10 min, 43-55-85% H₂O/MeOH/0.1% NH₄OH, flow 30 mL/min ((loading pump 4 mL MeOH/0.1% NH₄OH), column: XBridge BEH C18 100×19 mm, 5 microM) to obtain N-(4-amino-2-tetrahydropyran-2-yl-pyrazolo[4,3-c]pyridin-7-yl)-N′-[(4-fluoro-2-methyl-phenyl)methyl]-N′-isobutyl-oxamide (46.4 mg, 96.16 μmol, 14.98% yield) as a yellow gum.

LCMS (ESI): [M+H]⁺m/z: calcd 483.29; found 483.2; Rt=1.174 min.

### Step 6: The synthesis of N-(4-amino-1H-pyrazolo[4,3-c]pyridin-7-yl)-N′-[(4-fluoro-2-methyl-phenyl)methyl]-N′-isobutyl-oxamide

N-(4-amino-2-tetrahydropyran-2-yl-pyrazolo[4,3-c]pyridin-7-yl)-N′-[(4-fluoro-2-methyl-phenyl)methyl]-N′-isobutyl-oxamide (46.4 mg, 96.16 μmol) was dissolved in MeOH (1 mL) and HCl/dioxane (1 mL) was added thereto. The resulting solution was stirred for 1 hr and the reaction mixture was concentrated in vacuo. The residue was purified by HPLC (0-2-10 min, 0-75% H₂O/MeCN/0.1% FA, flow 30 mL/min ((loading pump 4 mL MeCN), target mass 380, column: Chromatorex SMB100-5T C18 100×19 mm, 5 microM) to obtain N-(4-amino-1H-pyrazolo[4,3-c]pyridin-7-yl)-N′-[(4-fluoro-2-methyl-phenyl)methyl]-N′-isobutyl-oxamide (15.2 mg, 34.20 μmol, 35.57% yield, HCOOH) as a light-yellow solid.

¹H NMR (600 MHz, DMSO-d₆) δ 0.74-0.92 (m, 6H), 1.93-2.01 (m, 1H), 2.19-2.33 (m, 3H), 3.07-3.10 (m, 1H), 3.26-3.27 (m, 1H), 4.23-4.83 (m, 2H), 6.59-6.91 (m, 2H), 6.94-7.11 (m, 2H), 7.17-7.29 (m, 1H), 7.48-7.74 (m, 1H), 8.14-8.20 (m, 1H), 9.53-10.53 (m, 1H), 12.41-13.52 (m, 1H).

LCMS (ESI): [M+H]⁺m/z: calcd 399.22; found 399.2; Rt=0.995 min.

### Example 60. Synthesis of N-(4-amino-1H-pyrazolo[4,3-c]pyridin-7-yl)-N′-isopropyl-N′-[[2-(trifluoromethyl)phenyl]methyl]oxamide (Compound 73)

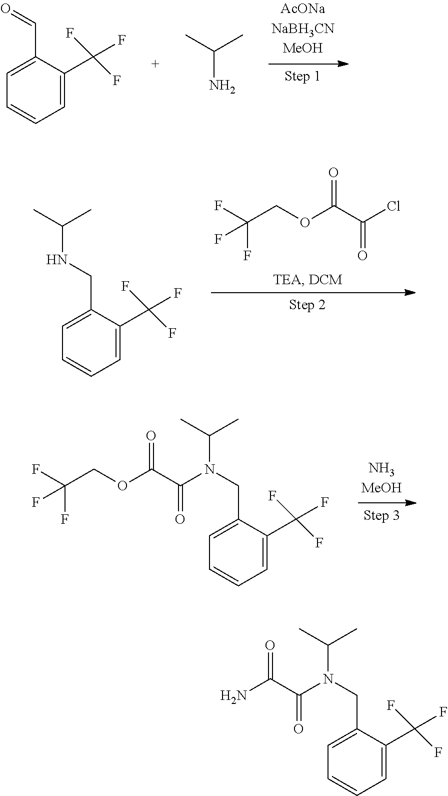

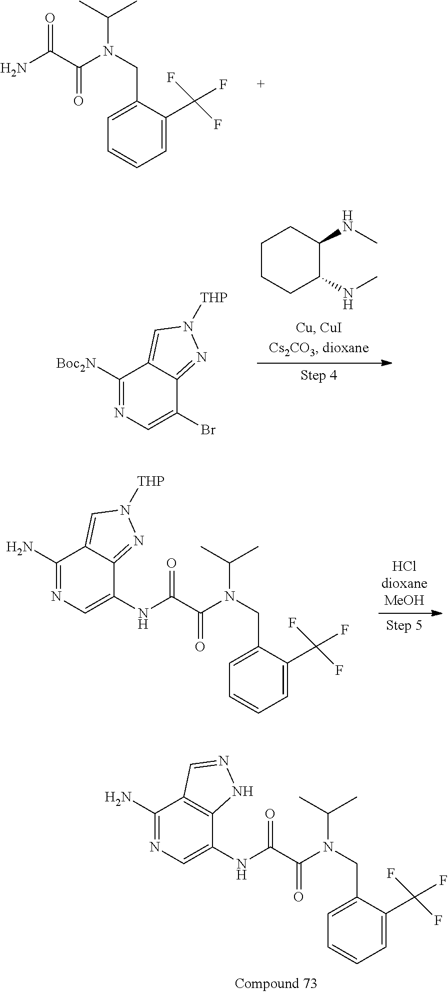

### Step 1: N-[[2-(trifluoromethyl)phenyl]methyl]propan-2-amine

2-(trifluoromethyl)benzaldehyde (2 g, 11.49 mmol, 1.51 mL) and propan-2-amine (678.96 mg, 11.49 mmol, 982.57 μL) were dissolved in MeOH (28.77 mL) and Sodium acetate (1.88 g, 22.97 mmol, 1.23 mL) was added thereto. The resulting mixture was stirred for 1 hr and Sodium cyanoborohydride (866.19 mg, 13.78 mmol) was added thereto. The resulting mixture was stirred overnight. The reaction mixture was concentrated in vacuo and aq. K₂CO₃ solution (15 mL) was added to the residue. The resulting mixture was extracted with DCM (2×10 mL) and combined organic layers were dried over anhydrous sodium sulfate, filtered and concentrated in vacuo to obtain N-[2-(trifluoromethyl)phenyl]methyl]propan-2-amine (2 g, 9.21 mmol, 80.15% yield) as a yellow oil.

LCMS (ESI): [M+H]⁺m/z: calcd 218.12; found 218.0; Rt=0.831 min.

### Step 2:2,2,2-trifluoroethyl 2-[isopropyl-[[2-(trifluoromethyl)phenyl]methyl]amino]-2-oxo-acetate

N-[[2-(trifluoromethyl)phenyl]methyl]propan-2-amine (2 g, 9.21 mmol) was dissolved in DCM (46.79 mL) and TEA (2.33 g, 23.02 mmol, 3.21 mL) was added. The reaction mixture was cooled and 2,2,2-trifluoroethyl 2-chloro-2-oxo-acetate (1.75 g, 9.21 mmol) was added dropwise. After that it was stirred at rt overnight. Then water (10 mL) was added, organic layer was stirred with brine and dried over anhydrous sodium sulfate, evaporated in vacuum to afford 2,2,2-trifluoroethyl 2-[isopropyl-[2-(trifluoromethyl)phenyl]methyl]amino]-2-oxo-acetate (3.2 g, 8.62 mmol, 93.61% yield) as a yellow oil.

LCMS (ESI): [M+H]⁺m/z: calcd 372.11; found 372.0; Rt=1.397 min.

### Step 3: N′-isopropyl-N′-[2-(trifluoromethyl)phenyl]methyl]oxamide

2,2,2-trifluoroethyl 2-[isopropyl-[2-(trifluoromethyl)phenyl]methyl]amino]-2-oxo-acetate (3.2 g, 8.62 mmol) was dissolved in NH3/MeOH (50 mL) and stirred overnight at rt. Then it was evaporated in vacuum and subjected to CC (CHCl₃-MeCN was used as an eluent mixture) to afford N′-isopropyl-N′-[2-(trifluoromethyl)phenyl]methyl]oxamide (1.7 g, 5.90 mmol, 68.42% yield) as a brown solid.

LCMS (ESI): [M+H]⁺m/z: calcd 289.12; found 289.0; Rt=1.226 min.

### Step 4: N-(4-amino-2-tetrahydropyran-2-yl-pyrazolo[4,3-c]pyridin-7-yl)-N′-isopropyl-N′-[[2-(trifluoromethyl)phenyl]methyl]oxamide

A mixture of tert-butyl N-(7-bromo-2-tetrahydropyran-2-yl-pyrazolo[4,3-c]pyridin-4-yl)-N-tert-butoxycarbonyl-carbamate (569.39 mg, 1.14 mmol), N′-isopropyl-N′-[2-(trifluoromethyl)phenyl]methyl]oxamide (0.3 g, 1.04 mmol), Cesium carbonate (508.62 mg, 1.56 mmol), rac-(1R,2R)—N1,N2-dimethylcyclohexane-1,2-diamine (118.43 mg, 832.57 μmol) and CuI (118.92 mg, 624.43 μmol, 21.16 μL) with a few mg of Cu(3.31 mg, 52.04 μmol) in dioxane (4.98 mL) was stirred in a sealed vial under argon at 105° C. for 48 hr. The resulting mixture was cooled down and filtered through silica gel to afford N-(4-amino-2-tetrahydropyran-2-yl-pyrazolo[4,3-c]pyridin-7-yl)-N′-isopropyl-N′-[[2-(trifluoromethyl)phenyl]methyl]oxamide (0.5 g, 991.07 μmol, 95.23% yield) as a brown solid.

LCMS (ESI): [M−H]⁻m/z: calcd 549.22; found 549.2; Rt=1.307 min.

### Step 5: The synthesis of N-(4-amino-1H-pyrazolo[4,3-c]pyridin-7-yl)-N′-isopropyl-N′-[[2-(trifluoromethyl)phenyl]methyl]oxamide

N-(4-amino-2-tetrahydropyran-2-yl-pyrazolo[4,3-c]pyridin-7-yl)-N′-isopropyl-N′-[2-(trifluoromethyl)phenyl]methyl]oxamide (0.5 g, 991.07 μmol) was dissolved in MeOH (10 mL) and dioxane/HCl (1.05 mmol, 5 mL) was added. Then mixture was stirred at rt overnight. The solution was evaporated and submitted to reverse phase HPLC (column: SunFire C18 100×19 mm, 5 μm; mobile phase: 20-45% 2-7.5 min water-MeCN+HCl, flow rate: 30 mL/min) to afford N-(4-amino-1H-pyrazolo[4,3-c]pyridin-7-yl)-N′-isopropyl-N′-[[2-(trifluoromethyl)phenyl]methyl]oxamide (16 mg, 3.8 μmol, 3.84% yield).

¹H NMR (500 MHz, DMSO-d₆) δ 0.69-1.17 (m, 6H), 4.16-4.96 (m, 3H), 5.59-7.33 (m, 3H), 7.38-7.59 (m, 2H), 7.63-7.77 (m, 2H), 8.07-8.23 (m, 1H), 9.32-14.15 (m, 1H).

LCMS (ESI): [M+H]⁺m/z: calcd 421.18; found 421.2; Rt=2.607 min.

### Example 61. Synthesis of N-(4-amino-1H-pyrazolo[4,3-c]pyridin-7-yl)-N′-benzyl-N′-[(5-fluoro-2-pyridyl)methyl]oxamide (Compound 68)

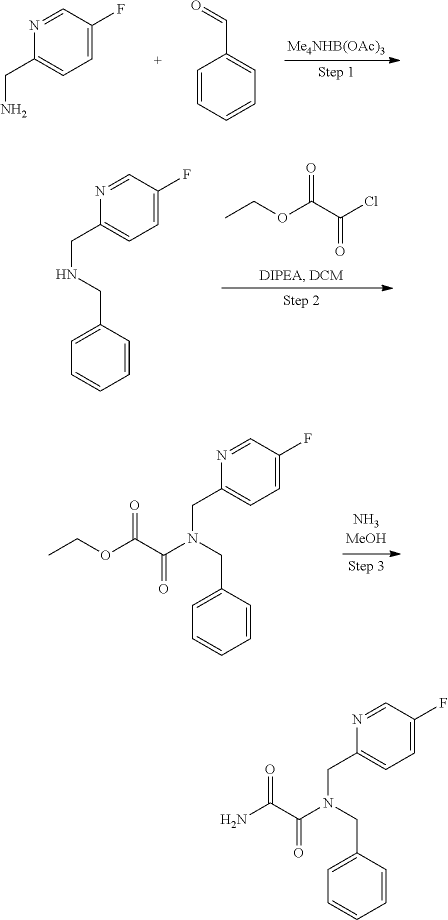

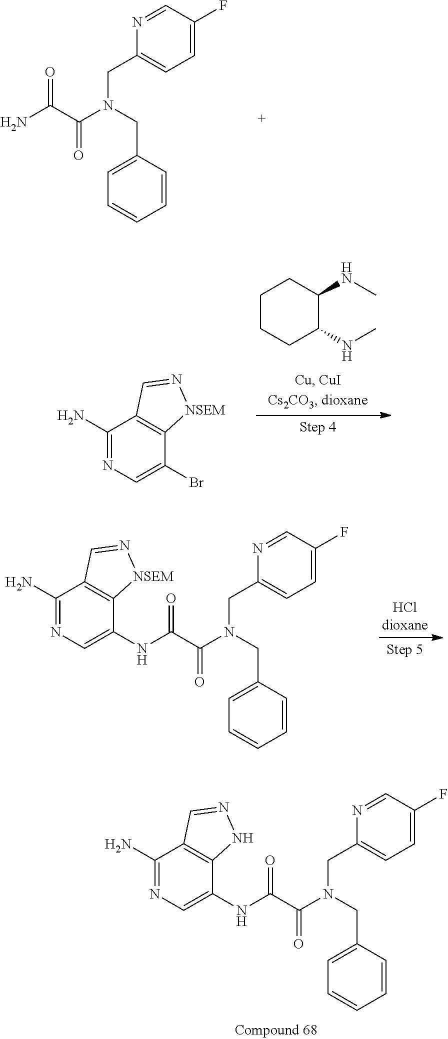

### Step 1: N-[(5-fluoro-2-pyridyl)methyl]-1-phenyl-methanamine

(5-fluoro-2-pyridyl)methanamine (4 g, 31.71 mmol), benzaldehyde (3.37 g, 31.71 mmol) and tetramethylammonium triacetoxyborohydride (16.69 g, 63.43 mmol) was mixed in DCM (50 mL) and stirred at RT overnight. Upon completion, the reaction mixture was washed with NaHCO₃ solution. Organic layer was dried over anhydrous sodium sulfate and concentrated under reduced pressure to afford N-[(5-fluoro-2-pyridyl)methyl]-1-phenyl-methanamine (5.3 g, crude) which was directly used in the next step without purification.

LCMS (ESI): [M+H]⁺m/z: calcd 217.14; found 217.0; Rt=0.470 min.

### Step 2: ethyl 2-[benzyl-[(5-fluoro-2-pyridyl)methyl]amino]-2-oxo-acetate

Ethyl 2-chloro-2-oxo-acetate (3.94 g, 28.83 mmol, 3.22 mL) was added dropwise to an ice bath cooled stirred solution of N-[(5-fluoro-2-pyridyl)methyl]-1-phenyl-methanamine (5.67 g, 26.21 mmol) and DIPEA (4.40 g, 34.08 mmol, 5.94 mL) in DCM (120 mL). The reaction mixture was stirred overnight. Upon completion, the reaction mixture was washed with water. The organic layer was separated, dried over anhydrous sodium sulfate, and concentrated under reduced pressure. The residue was purified by flash column chromatography (Interchim; 120 g SiO₂, CYCLOHEX-EtOAc from 0~100%, flow rate=70 mL/min, cv=9.7) to afford ethyl 2-[benzyl-[(5-fluoro-2-pyridyl)methyl]amino]-2-oxo-acetate (2.2 g, 6.95 mmol, 26.53% yield).

LCMS (ESI): [M+H]⁺m/z: calcd 317.13; found 317.0; Rt=1.339 min.

### Step 3: N′-benzyl-N′-[(5-fluoro-2-pyridyl)methyl]oxamide

Ethyl 2-[benzyl-[(5-fluoro-2-pyridyl)methyl]amino]-2-oxo-acetate (2.2 g, 6.95 mmol) was dissolved in saturated NH3/methanol solution and stirred overnight. Upon completion, the reaction mixture was concentrated under reduced pressure to afford N′-benzyl-N′-[(5-fluoro-2-pyridyl)methyl]oxamide (1.95 g, crude) which was used in the next step without purification.

LCMS (ESI): [M+H]⁺m/z: calcd 288.12; found 288.2; Rt=1.083 min.

### Step 4: N-[4-amino-1-(2-trimethylsilylethoxymethyl)pyrazolo[4,3-c]pyridin-7-yl]-N′-benzyl-N′-[(5-fluoro-2-pyridyl)methyl]oxamide

N′-benzyl-N′-[(5-fluoro-2-pyridyl)methyl]oxamide (0.3 g, 1.04 mmol), 7-bromo-1-(2-trimethylsilylethoxymethyl)pyrazolo[4,3-c]pyridin-4-amine (358.48 mg, 1.04 mmol), copper (39.65 mg, 623.88 μmol), Copper (I) iodide (198.88 mg, 1.04 mmol, 35.39 μL), (1S,2S)-N,N′-Bis -methyl-1,2-cyclohexane-diamine (148.53 mg, 1.04 mmol, 164.67 μL) were mixed in dioxane (10 mL). The reaction mixture was stirred under Ar atmosphere at 100° C. for 48 hr. Upon completion, the mixture was diluted with water and extracted with DCM. Organic layer was sashed with aq. ammonia, separated, dried over anhydrous sodium sulfate, and concentrated under reduced pressure. The residue was submitted to HPLC (2-10 min 40-100% MeOH+FA 30 mL/min) to afford N-[4-amino-1-(2-trimethylsilylethoxymethyl)pyrazolo[4,3-c]pyridin-7-yl]-N′-benzyl-N′-[(5-fluoro-2-pyridyl)methyl]oxamide (0.13 g, 236.51 μmol, 22.65% yield).

LCMS (ESI): [M+H]⁺m/z: calcd 550.28; found 550.0; Rt=1.420 min.

### Step 5: The synthesis of N-(4-amino-1H-pyrazolo[4,3-c]pyridin-7-yl)-N′-benzyl-N′-[(5-fluoro-2-pyridyl)methyl]oxamide

N-[4-amino-1-(2-trimethylsilylethoxymethyl)pyrazolo[4,3-c]pyridin-7-yl]-N′-benzyl-N′-[(5-fluoro-2-pyridyl)methyl]oxamide (0.13 g, 236.51 μmol) was dissolved in HCl/dioxane solution (2 mL) and stirred at 20° C. for 4 hr. Upon completion, the reaction mixture was concentrated under reduced pressure and the residue was purified by reverse phase chromatography (0-2-10 min 0-0-75% H₂O/ACN/0.1% FA, flow 30 mL/min ((loading pump 4 mL ACN) column: Chromatorex SMB100-5T C18 100×19 mm, 5 microM) to afford N-(4-amino-1H-pyrazolo[4,3-c]pyridin-7-yl)-N′-benzyl-N′-[(5-fluoro-2-pyridyl)methyl]oxamide (0.033 g, 71.51 μmol, 30.24% yield, HCCO—) as a yellow solid.

¹H NMR (600 MHz, DMSO-d₆) δ 4.48-4.61 (m, 2H), 4.80-4.97 (m, 2H), 6.66-6.98 (m, 2H), 7.24-7.30 (m, 2H), 7.30-7.35 (m, 2H), 7.36-7.38 (m, 1H), 7.38-7.51 (m, 1H), 7.62-7.67 (m, 1H), 7.67-7.76 (m, 1H), 8.16-8.30 (m, 1H), 8.46-8.55 (m, 1H), 9.66-10.69 (m, 1H), 12.48-13.39 (m, 1H).

LCMS (ESI): [M+H]⁺m/z: calcd 420.17; found 420.2; Rt=2.327 min.

### Example 62. Synthesis of N-(4-amino-1H-pyrazolo[4,3-c]pyridin-7-yl)-N′-benzyl-N′-[[6-(trifluoromethyl)-2-pyridyl]methyl]oxamide (Compound 55)

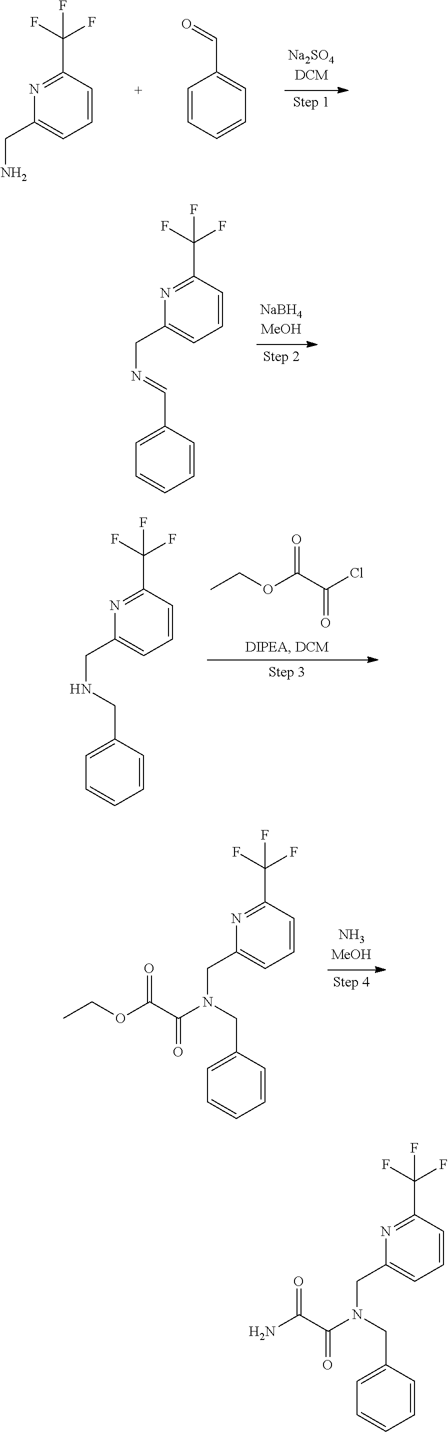

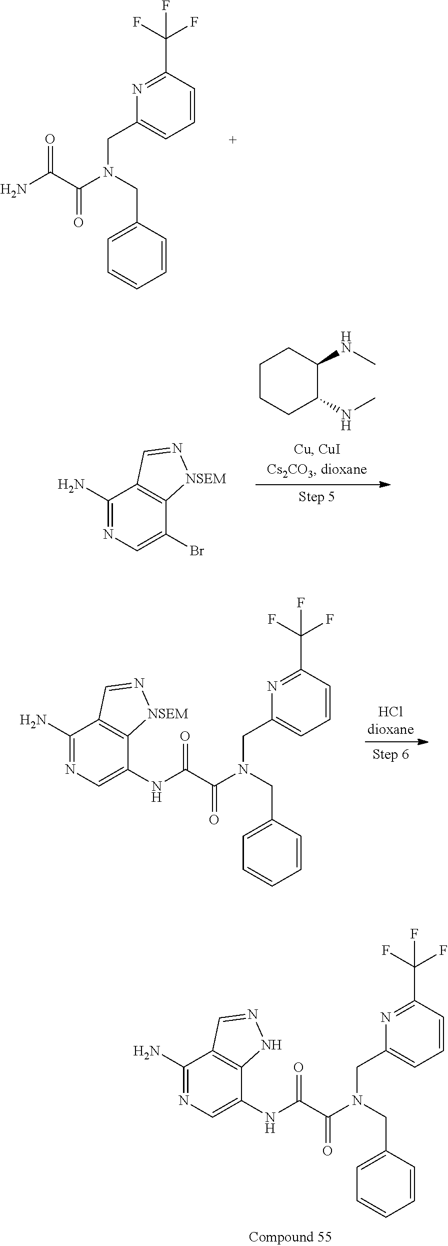

### Step 1: (E)-1-phenyl-N-[[6-(trifluoromethyl)-2-pyridyl]methyl]methanimine

[6-(trifluoromethyl)-2-pyridyl]methanamine (3.5 g, 19.87 mmol), benzaldehyde (2.11 g, 19.87 mmol) and Sodium sulfate, anhydrous (5.64 g, 39.74 mmol, 2.11 mL) was mixed in DCM (59.75 mL) and stirred at RT overnight. Upon completion, the reaction mixture was filtered and filtrate was concentrated under reduced pressure to afford (E)-1-phenyl-N-[6-(trifluoromethyl)-2-pyridyl]methyl]methanimine (4.67 g, crude) which was directly used in the next step without purification and analytical data collection.

### Step 2:1-phenyl-N-[[6-(trifluoromethyl)-2-pyridyl]methyl]methanamine

Sodium Borohydride (1.00 g, 26.51 mmol, 933.82 μL) was added portionwise to a stirred solution of (E)-1-phenyl-N-[6-(trifluoromethyl)-2-pyridyl]methyl]methanimine (4.67 g, 17.67 mmol) in methanol (50 mL). The reaction mixture was stirred overnight and concentrated under reduced pressure. The residue was taken up in DCM, washed with NaHCO₃ solution, dried over anhydrous sodium sulfate and concentrated to afford 1-phenyl-N-[6-(trifluoromethyl)-2-pyridyl]methyl]methanamine (4.2 g, crude) which was used in the next step without purification.

LCMS (ESI): [M+H]⁺m/z: calcd 267.11; found 267.2; Rt=0.785 min.

### Step 3: ethyl 2-[benzyl-[[6-(trifluoromethyl)-2-pyridyl]methyl]amino]-2-oxo-acetate

Ethyl 2-chloro-2-oxo-acetate (2.37 g, 17.35 mmol, 1.94 mL) was added dropwise to an ice bath cooled stirred solution of 1-phenyl-N-[[6-(trifluoromethyl)-2-pyridyl]methyl]methanamine (4.2 g, 15.77 mmol) and DIPEA (2.65 g, 20.51 mmol, 3.57 mL) in DCM (60 mL). The reaction mixture was stirred overnight. Upon completion, the reaction mixture was washed with water. The organic layer was separated, dried over anhydrous sodium sulfate and concentrated under reduced pressure to afford ethyl 2-[benzyl-[[6-(trifluoromethyl)-2-pyridyl]methyl]amino]-2-oxo-acetate (4.7 g, crude) which was used in the next step without purification.

LCMS (ESI): [M+H]⁺m/z: calcd 367.13; found 367.0; Rt=1.510 min.

### Step 4: N′-benzyl-N′-[[6-(trifluoromethyl)-2-pyridyl]methyl]oxamide

Ethyl 2-[benzyl-[[6-(trifluoromethyl)-2-pyridyl]methyl]amino]-2-oxo-acetate (4.1 g, 11.19 mmol) was dissolved in saturated NH3/methanol solution and stirred overnight. Upon completion, the reaction mixture was concentrated under reduced pressure to afford N′-benzyl-N′-[6-(trifluoromethyl)-2-pyridyl]methyl]oxamide (3.55 g, crude).

LCMS (ESI): [M−H]⁻m/z: calcd 338.11; found 338.2; Rt=1.259 min.

### Step 5: N-[4-amino-1-(2-trimethylsilylethoxymethyl)pyrazolo[4,3-c]pyridin-7-yl]-N′-benzyl-N′-[[6-(trifluoromethyl)-2-pyridyl]methyl]oxamide

N′-benzyl-N′-[6-(trifluoromethyl)-2-pyridyl]methyl]oxamide (500.00 mg, 1.48 mmol), 7-bromo-1-(2-trimethylsilylethoxymethyl)pyrazolo[4,3-c]pyridin-4-amine (508.89 mg, 1.48 mmol), copper (0.05 g, 786.78 μmol), Copper (I) iodide (282.32 mg, 1.48 mmol, 50.23 μL), (1S,2S)-N,N′-Bis -methyl-1,2-cyclohexane-diamine (210.85 mg, 1.48 mmol, 233.76 μL) were mixed in dioxane (15 mL). The reaction mixture was stirred under Ar atmosphere at 100° C. for 48 hr. Upon completion, the mixture was diluted with water and extracted with DCM. Organic layer was separated, dried over anhydrous sodium sulfate, and concentrated under reduced pressure. The residue was submitted to HPLC (2-10 min 45-75% MeOH+FA 30 mL/min) to afford N-[4-amino-1-(2-trimethylsilylethoxymethyl)pyrazolo[4,3-c]pyridin-7-yl]-N′-benzyl-N′-[[6-(trifluoromethyl)-2-pyridyl]methyl]oxamide (0.14 g, 233.46 μmol, 15.75% yield).

LCMS (ESI): [M+H]⁺m/z: calcd 600.28; found 600.3; Rt=1.393 min.

### Step 6: The synthesis of N-(4-amino-1H-pyrazolo[4,3-c]pyridin-7-yl)-N′-benzyl-N′-[[6-(trifluoromethyl)-2-pyridyl]methyl]oxamide

N-[4-amino-1-(2-trimethylsilylethoxymethyl)pyrazolo[4,3-c]pyridin-7-yl]-N′-benzyl-N′-[6-(trifluoromethyl)-2-pyridyl]methyl]oxamide (0.14 g, 233.46 μmol) was dissolved in HCl/dioxane solution (w (HCl)=13%) and stirred at 20° C. for 5 hr. Upon completion, the reaction mixture was concentrated under reduced pressure and the residue was submitted to HPLC (2-10 min 0-85% ACN+FA, 30 mL/min ((loading pump 4 mL ACN) column: Cromatorex C18 100×19, 5 microM) to afford N-(4-amino-1H-pyrazolo[4,3-c]pyridin-7-yl)-N′-benzyl-N′-[[6-(trifluoromethyl)-2-pyridyl]methyl]oxamide (0.06 g, 127.82 μmol, 54.75% yield) as a beige solid.

¹H NMR (600 MHz, DMSO-d₆) δ 4.18-5.10 (m, 4H), 6.05-7.02 (m, 2H), 7.05-7.41 (m, 5H), 7.55-7.71 (m, 2H), 7.72-7.86 (m, 1H), 7.97-8.11 (m, 1H), 8.14-8.24 (m, 1H), 9.77-10.70 (m, 1H), 12.59-13.47 (m, 1H).

LCMS (ESI): [M+H]⁺m/z: calcd 470.17; found 470.2; Rt=2.791 min.

### Example 63. Synthesis of N-(4-Amino-2H-pyrazolo[4,3-c]pyridin-7-yl)-N′-cyclobutyl-N′-[[5-(trifluoromethyl)-2-pyridyl]methyl]oxamide (Compound 61)

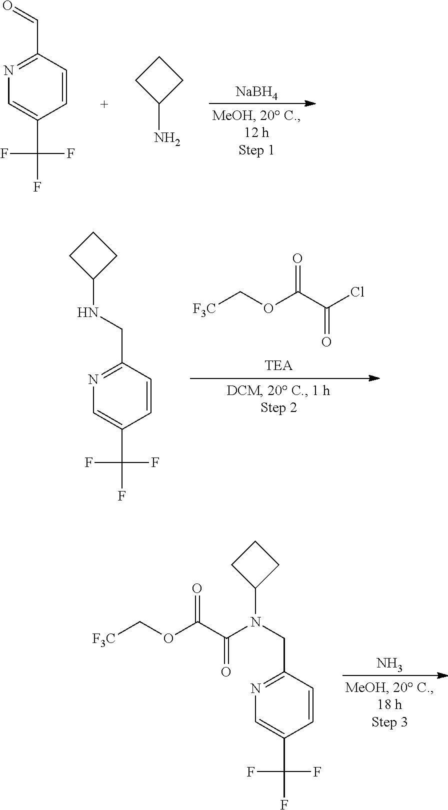

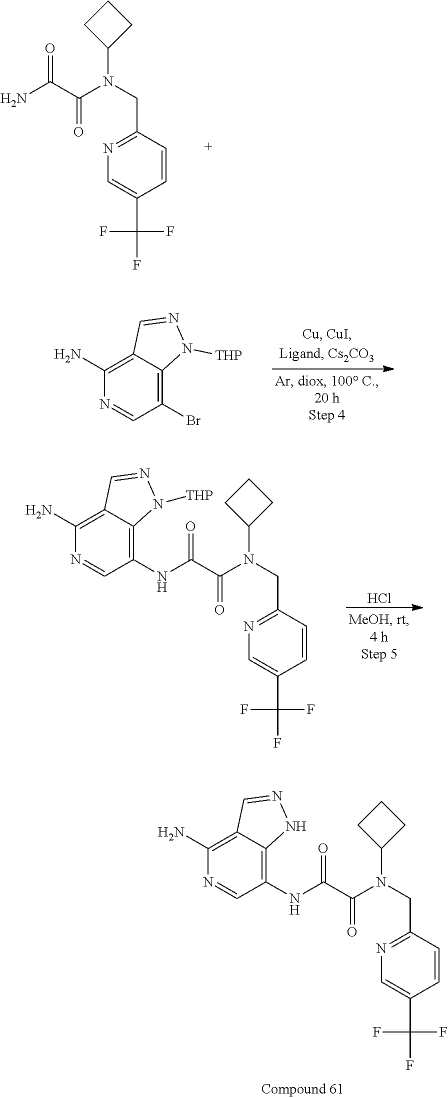

### Step 1: The Synthesis of N-[[5-(Trifluoromethyl)-2-pyridyl]methyl]cyclobutanamine

A solution of 5-(trifluoromethyl)pyridine-2-carbaldehyde (0.7 g, 4.00 mmol) and cyclobutanamine (284.31 mg, 4.00 mmol, 341.31 μL) in MeOH (19.42 mL) was stirred at 20° C. for 12 h. To this solution, Sodium Borohydride (302.45 mg, 8.00 mmol, 281.61 μL) was added and the resulting mixture was stirred for 5 hr. The solvent was removed in vacuo, the residue was taken up with water (40 mL) and extracted with DCM (3*20 mL). The combined organic layer was washed with brine (10 mL), dried over Na₂SO₄ and evaporated to obtain N-[[5-(trifluoromethyl)-2-pyridyl]methyl]cyclobutanamine (0.7 g, 3.04 mmol, 76.06% yield).

LCMS (ESI): [M+H]⁺m/z: calcd 231.2; found 231.2; Rt=0.331 min.

### Step 2: The Synthesis of 2,2,2-Trifluoroethyl 2-[cyclobutyl-[[5-(trifluoromethyl)-2-pyridyl]methyl]amino]-2-oxo-acetate

2,2,2-Trifluoroethyl 2-chloro-2-oxo-acetate (231.69 mg, 1.22 mmol) was added dropwise to the ice-cooled solution of N-[[5-(trifluoromethyl)-2-pyridyl]methyl]cyclobutanamine (200 mg, 868.70 μmol) and Triethylamine (175.81 mg, 1.74 mmol, 242.16 μL) in Dichloromethane (10 mL). After addition was complete, cooling bath was removed and resulting mixture was allowed to warm up to 20° C. and stirred for 1 hr. Then, 10% aq. NaHCO₃ solution (10 mL) was added and stirring was continued for 10 minutes. Then, organic layer was separated, dried over Na₂SO₄ and concentrated under reduced pressure, leaving 2,2,2-trifluoroethyl 2-[cyclobutyl-[5-(trifluoromethyl)-2-pyridyl]methyl]amino]-2-oxo-acetate (0.33 g, 858.76 μmol, 98.86% yield).

LCMS (ESI): [M+H]⁺m/z: calcd 385.2; found 385.2; Rt=1.066 min.

### Step 3: The Synthesis of N′-Cyclobutyl-N′-[[5-(trifluoromethyl)-2-pyridyl]methyl]oxamide

2,2,2-Trifluoroethyl 2-[cyclobutyl-[5-(trifluoromethyl)-2-pyridyl]methyl]amino]-2-oxo-acetate (0.33 g, 858.76 μmol) was dissolved in Ammonia (7N in methanol, 15,3% w/w)(7.79 g, 457.42 mmol, 10 mL). Resulting reaction mixture was stirred at 20° C. for 18 hr. Then, volatiles were removed under reduced pressure, leaving N′-cyclobutyl-N′-[5-(trifluoromethyl)-2-pyridyl]methyl]oxamide (0.26 g, crude).

LCMS (ESI): [M+H]⁺m/z: calcd 302.0; found 302.0; Rt=0.736 min.

### Step 4: The Synthesis of N-(4-Amino-2-tetrahydropyran-2-yl-pyrazolo[4,3-c]pyridin-7-yl)-N′-cyclobutyl-N′-[[5-(trifluoromethyl)-2-pyridyl]methyl]oxamide

N′-Cyclobutyl-N′-[5-(trifluoromethyl)-2-pyridyl]methyl]oxamide (260 mg, 863.03 μmol), 7-bromo-2-tetrahydropyran-2-yl-pyrazolo[4,3-c]pyridin-4-amine (307.74 mg, 1.04 mmol), Copper (5.48 mg, 86.30 μmol), Copper (I) iodide (82.18 mg, 431.52 μmol, 14.62 μL), (S,S)-(+)—N,N′-Dimethyl-1,2-cyclohexanediamine (61.38 mg, 431.52 μmol, 68.05 μL) and Potassium Carbonate (178.92 mg, 1.29 mmol, 78.13 μL) were mixed together in Dioxane (4 mL). Reaction flask was purged with argon and resulting mixture was stirred at 100° C. for 20 hr under inert atmosphere. Then, it was diluted with DCM (15 mL) and filtered. Filtrate was concentrated under reduced pressure, leaving N-(4-amino-2-tetrahydropyran-2-yl-pyrazolo[4,3-c]pyridin-7-yl)-N′-cyclobutyl-N′-[5-(trifluoromethyl)-2-pyridyl]methyl]oxamide (0.55 g, crude).

LCMS (ESI): [M+H]⁺m/z: calcd 518.0; found 518.0; Rt=1.187 min.

### Step 5: The Synthesis of N-(4-Amino-2H-pyrazolo[4,3-c]pyridin-7-yl)-N′-cyclobutyl-N′-[[5-(trifluoromethyl)-2-pyridyl]methyl]oxamide (Compound 61)

Hydrogen chloride solution 4.0M in dioxane (1.01 g, 2.77 mmol, 1 mL, 10% purity) was added to the solution of N-(4-amino-2-tetrahydropyran-2-yl-pyrazolo[4,3-c]pyridin-7-yl)-N′-cyclobutyl-N′-[[5-(trifluoromethyl)-2-pyridyl]methyl]oxamide (0.55 g, 425.12 μmol) in Methanol (3 mL). Resulting mixture was stirred at 25° C. for 4 hr. Then, volatiles were removed under reduced pressure and residue was subjected to HPLC (May 5, 2030% 0-1.3-5.3 min H₂O/ACN/0.1% FA, flow 30 mL/min, column: Chromatorex 18 SMB 100-ST 100*19 mm 5 μm), affording 105 mg of N-(4-amino-2H-pyrazolo[4,3-c]pyridin-7-yl)-N′-cyclobutyl-N′-[5-(trifluoromethyl)-2-pyridyl]methyl]oxamide (2 mg, 4.17 μmol, 9.81e-1% yield, HCOOH). 2 mg of this was shipped and the rest was re-purified (0-5-30% 0-1.3-7.3 min H₂O/ACN, flow: 30 ml/min, column: Chromatorex 18 SNB100-5T 100*19 mm 5 μm), affording N-(4-amino-2H-pyrazolo[4,3-c]pyridin-7-yl)-N′-cyclobutyl-N′-[5-(trifluoromethyl)-2-pyridyl]methyl]oxamide (19.7 mg, 45.46 μmol, 10.69% yield).

¹H NMR (600 MHz, dmso) δ 1.53-2.09 (m, 5H), 2.11-2.21 (m, 1H), 4.48-5.16 (m, 3H), 6.57-7.28 (m, 2H), 7.42-7.57 (m, 1H), 7.58-7.82 (m, 1H), 8.13-8.41 (m, 2H), 8.72-8.98 (m, 1H), 9.69-10.61 (m, 1H), 11.62-14.28 (m, 2H).

LCMS (ESI): [M+H]⁺m/z: calcd 434.0; found 434.0; Rt=2.237 min.

### Example 64. Synthesis of rac-N1-(4-amino-1H-pyrazolo[4,3-c]pyridin-7-yl)-N2-methyl-N2-(1-(4-(trifluoromethyl)phenyl)ethyl)oxalamide (Compound 85), (S)-N1-(4-amino-1H-pyrazolo[4,3-c]pyridin-7-yl)-N2-methyl-N2-(1-(4-(trifluoromethyl)phenyl)ethyl)oxalamide (Compound 53) & (R)—N¹-(4-amino-1HI-pyrazolo[4,3-c]pyridin-7-yl)-N2-methyl-N2-(1-(4-(trifluoromethyl)phenyl)ethyl)oxalamide (Compound 78)

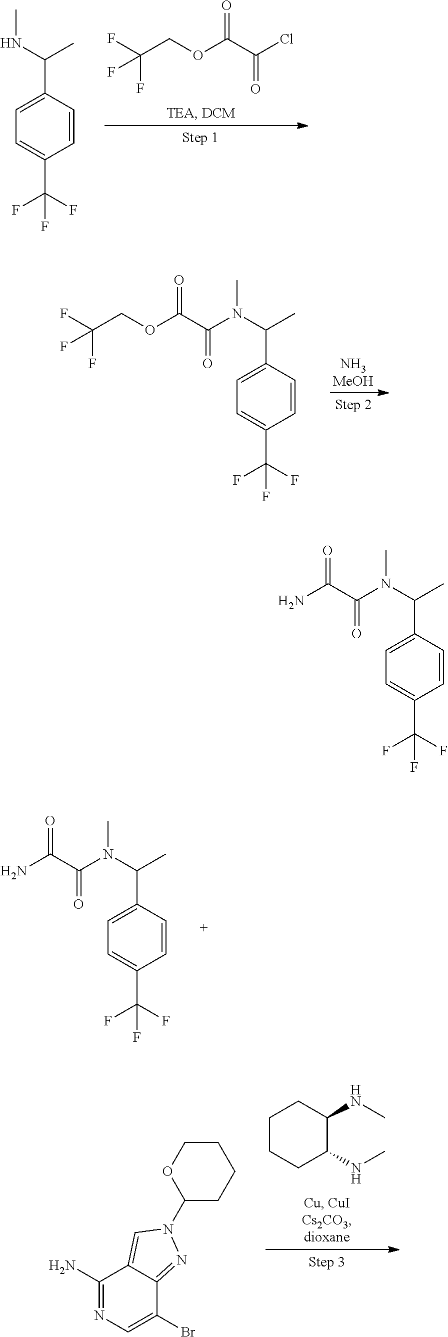

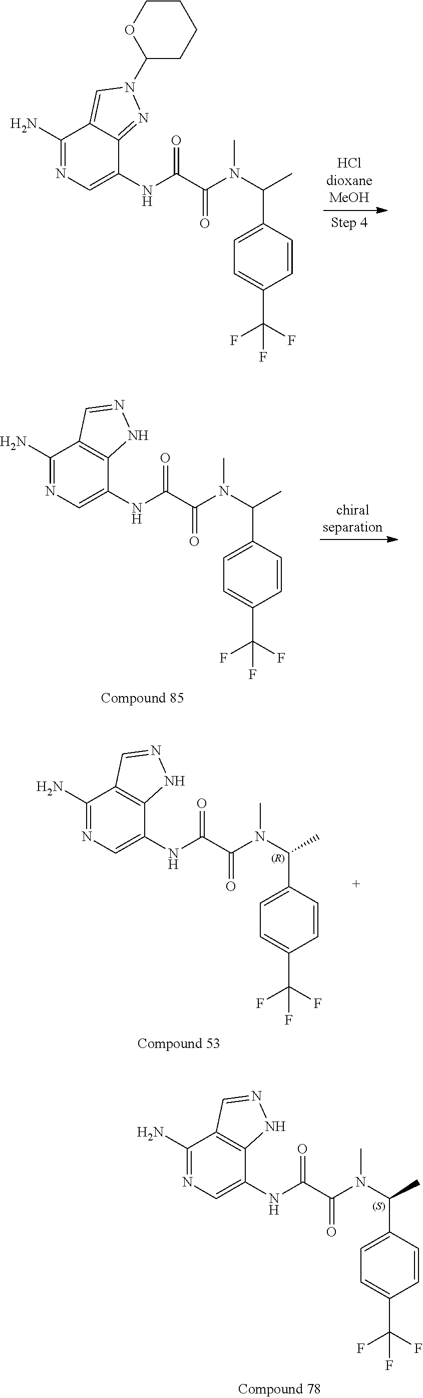

### Step 1:2,2,2-trifluoroethyl 2-[methyl-[1-[4-(trifluoromethyl)phenyl]ethyl]amino]-2-oxo-acetate

N-methyl-1-[4-(trifluoromethyl)phenyl]ethanamine (502.3 mg, 2.47 mmol) and Triethylamine (275.15 mg, 2.72 mmol, 378.99 μL) were dissolved in DCM (12 mL) and the resulting solution was cooled to −5° C. in an ice/methanol bath. 2,2,2-trifluoroethyl 2-chloro-2-oxo-acetate (494.46 mg, 2.60 mmol) was added dropwise and the resulting mixture was stirred overnight. Water (15 mL) was added to the reaction mixture and an organic layer was separated. The aqueous layer was extracted with DCM (15 mL) and combined organic layers were dried over anhydrous sodium sulfate, filtered, and concentrated in vacuo to obtain 2,2,2-trifluoroethyl 2-[methyl-[1-[4-(trifluoromethyl)phenyl]ethyl]amino]-2-oxo-acetate (811 mg, 2.27 mmol, 91.84% yield) as a light-yellow oil.

LCMS (ESI): [M+H]⁺m/z: calcd 358.09; found 358.2; Rt=1.344 min.

### Step 2: N′-methyl-N′-[1-[4-(trifluoromethyl)phenyl]ethyl]oxamide

2,2,2-trifluoroethyl 2-[methyl-[1-[4-(trifluoromethyl)phenyl]ethyl]amino]-2-oxo-acetate (811 mg, 2.27 mmol) was dissolved in MeOH (10 mL) and NH3/MeOH (15 mL) was added thereto. The resulting solution was stirred overnight. The reaction mixture was concentrated in vacuo to obtain N′-methyl-N′-[1-[4-(trifluoromethyl)phenyl]ethyl]oxamide (628 mg, crude).

LCMS (ESI): [M−H]⁻m/z: calcd 273.08; found 273.0; Rt=1.224 min.

### Step 3: N-(4-amino-2-tetrahydropyran-2-yl-pyrazolo[4,3-c]pyridin-7-yl)-N′-methyl-N′-[1-[4-(trifluoromethyl)phenyl]ethyl]oxamide

All components were partitioned between two 8 ml vials. N′-methyl-N′-[1-[4-(trifluoromethyl)phenyl]ethyl]oxamide (300 mg, 1.09 mmol), 7-bromo-2-tetrahydropyran-2-yl-pyrazolo[4,3-c]pyridin-4-amine (357.57 mg, 1.20 mmol), Copper (3.48 mg, 54.70 μmol), Copper (I) iodide (104.17 mg, 546.97 μmol, 18.54 μL, rac-(1R,2R)—N1,N2-dimethylcyclohexane-1,2-diamine (116.70 mg, 820.45 μmol) and Cesium carbonate (712.85 mg, 2.19 mmol) were mixed in Dioxane (8 mL). The resulting mixture was splurged with argon for 5 min. The vials were sealed and heated at 100° C. over weekend. The reaction mixture was cooled and filtered. The filter cake was washed with MeOH (10 mL) and the filtrate was concentrated in vacuo to obtain N-(4-amino-2-tetrahydropyran-2-yl-pyrazolo[4,3-c]pyridin-7-yl)-N′-methyl-N′-[1-[4-(trifluoromethyl)phenyl]ethyl]oxamide (1 g, crude) as a greenish solid which was used in the next step without purification.

LCMS (ESI): [M+H]⁺m/z: calcd 491.2; found 491.2; Rt=1.167 min.

### Step 4: The synthesis of N-(4-amino-1H-pyrazolo[4,3-c]pyridin-7-yl)-N′-methyl-N′-[1-[4-(trifluoromethyl)phenyl]ethyl]oxamide

N-(4-amino-2-tetrahydropyran-2-yl-pyrazolo[4,3-c]pyridin-7-yl)-N′-methyl-N′-[1-[4-(trifluoromethyl)phenyl]ethyl]oxamide (1 g, 2.04 mmol) was dissolved in MeOH (5 mL) and HCl/Dioxane (5 mL) was added thereto. The resulting mixture was stirred for 3 hr and then concentrated in vacuo. The residue was purified by HPLC (0-2-10 min, Aug. 15, 1955 H₂O/MeOH/0.1NH4OH, flow 30 mL/min ((loading pump 4 mL MeOH), target mass 407, column: XBridge BEH C18 100×19 mm, 5 microM) to obtain N-(4-amino-1H-pyrazolo[4,3-c]pyridin-7-yl)-N′-methyl-N′-[1-[4-(trifluoromethyl)phenyl]ethyl]oxamide (43.3 mg, 106.56 μmol, 5.23% yield) as a light-yellow solid.

¹H NMR (600 MHz, dmso) δ 1.29-1.67 (m, 3H), 2.57-2.92 (m, 3H), 5.36-5.87 (m, 1H), 6.61-6.91 (m, 2H), 7.49-7.59 (m, 1H), 7.61-7.65 (m, 1H), 7.65-7.71 (m, 1H), 7.72-7.78 (m, 2H), 8.13-8.21 (m, 1H), 9.65-10.73 (m, 1H), 12.47-13.25 (m, 1H).

LCMS (ESI): [M+H]⁺m/z: calcd 407.16; found 407.2; Rt=1.010 min.

### Step 5: Synthesis of(S)-N1-(4-amino-1H-pyrazolo[4,3-c]pyridin-7-yl)-N2-methyl-N2-(1-(4-(trifluoromethyl)phenyl)ethyl)oxalamide (Compound 53) & (R)—N¹-(4-amino-1H-pyrazolo[4,3-c]pyridin-7-yl)-N2-methyl-N2-(1-(4-(trifluoromethyl)phenyl)ethyl)oxalamide (Compound 78)

N-(4-amino-1H-pyrazolo[4,3-c]pyridin-7-yl)-N′-methyl-N′-[1-[4-(trifluoromethyl)phenyl]ethyl]oxamide (36.9 mg, 90.81 μmol) was chirally separated (Column: Chiralcel OJ-H (250×20 mm, 5 mkm); Mobile phase: Hexane (0.1% DEA)-IPA-MeOH, 80-10-10. Flow Rate: 12 mL/min; Column Temperature: 24′C; Wavelength: 205 nm; RetTime=59.37 min) to obtain(S)-N1-(4-amino-1H-pyrazolo[4,3-c]pyridin-7-yl)-N2-methyl-N2-(1-(4-(trifluoromethyl)phenyl)ethyl)oxalamide (14.59 mg, 35.90 μmol, 79.08% yield) as a light-yellow solid.

¹H NMR (600 MHz, DMSO-d₆) § 1.27-1.67 (m, 3H), 2.59-2.92 (m, 3H), 5.32-5.85 (m, 1H), 6.51-6.80 (m, 2H), 7.45-7.83 (m, 5H), 8.06-8.24 (m, 1H), 9.63-10.62 (m, 1H), 12.54-13.37 (m, 1H).

LCMS (ESI): [M+H]⁺m/z: calcd 407.16; found 407.0; Rt=2.265 min.

N-(4-amino-1H-pyrazolo[4,3-c]pyridin-7-yl)-N′-methyl-N′-[1-[4-(trifluoromethyl)phenyl]ethyl]oxamide (36.9 mg, 90.81 μmol) was chirally separated (Column: Chiralcel OJ-H (250×20 mm, 5 mkm); Mobile phase: Hexane (0.1% DEA)-IPA-MeOH, 80-10-10. Flow Rate: 12 mL/min; Column Temperature: 24′C; Wavelength: 205 nm; RetTime=39.73 min) to obtain (R)—N¹-(4-amino-1H-pyrazolo[4,3-c]pyridin-7-yl)-N2-methyl-N2-(1-(4-(trifluoromethyl)phenyl)ethyl)oxalamide (15.95 mg, 39.25 μmol, 86.45% yield) as a light-yellow solid.

¹H NMR (600 MHz, dmso) δ 1.25-1.71 (m, 3H), 2.74 (d, 3H), 5.36-5.84 (m, 1H), 6.60-6.85 (m, 2H), 6.89-7.68 (m, 3H), 7.69-7.78 (m, 2H), 8.12-8.22 (m, 1H), 9.65-10.67 (m, 1H), 12.62-13.36 (m, 1H).

LCMS (ESI): [M+H]⁺m/z: calcd 407.16; found 407.0; Rt=2.269 min.

The absolute stereochemistry of the two compounds was independently confirmed.

### Example 65. Synthesis of N-(4-amino-1H-pyrazolo[4,3-c]pyridin-7-yl)-N′-(m-tolylmethyl)-N′-(2-pyridylmethyl)oxamide (Compound 52)

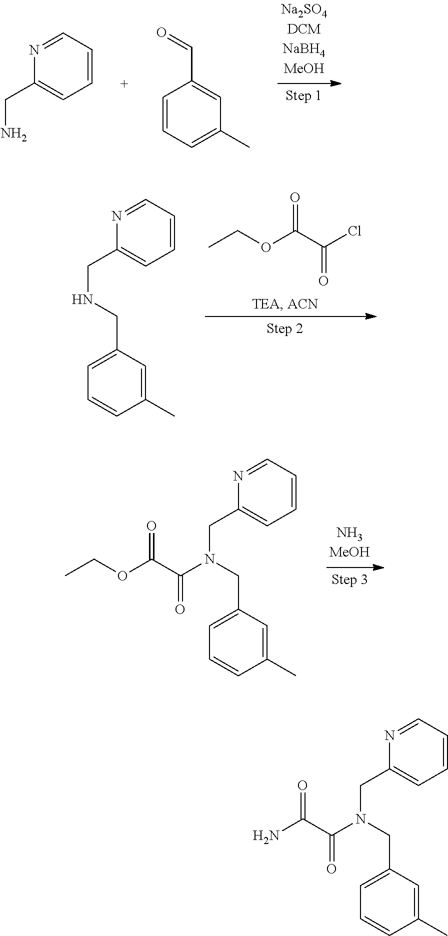

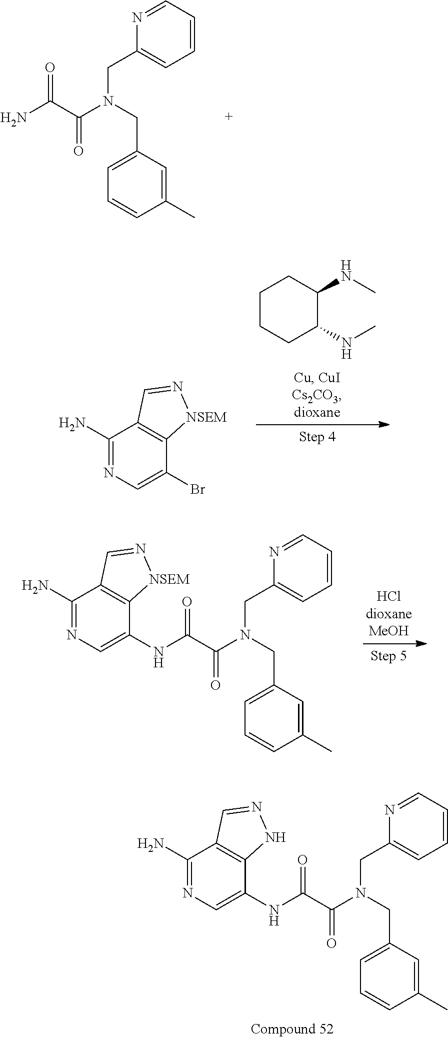

### Step 1:1-(m-tolyl)-N-(2-pyridylmethyl)methanamine

2-pyridylmethanamine (1 g, 9.25 mmol, 953.29 μL), Sodium sulfate, anhydrous (19.70 g, 138.71 mmol, 7.35 mL) and 3-methylbenzaldehyde (1.11 g, 9.25 mmol, 1.09 mL) were mixed in DCM (16.57 mL) at 20° C. The resulting mixture was stirred at 20° C. for 12 hr, then filtered and concentrated in vacuo. The residue was dissolved in methanol (16.57 mL), cooled to 5° C. and Sodium Borohydride (384.80 mg, 10.17 mmol, 358.29 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford 1-(m-tolyl)-N-(2-pyridylmethyl)methanamine (1.4 g, crude) as a brown oil.

LCMS (ESI): [M+H]⁺m/z: calcd 213.14; found 213.2; Rt=0.730 min.

### Step 2: ethyl 2-[m-tolylmethyl(2-pyridylmethyl)amino]-2-oxo-acetate

1-(m-tolyl)-N-(2-pyridylmethyl)methanamine (1.4 g, 3.96 mmol) and TEA (800.79 mg, 7.91 mmol, 1.10 mL) were dissolved in acetonitrile (20 mL), cooled with ice-water bath and then ethyl 2-chloro-2-oxoacetate (756.34 mg, 5.54 mmol, 618.94 μL) was added. Solution was stirred overnight at 20° C. The RM was concentrated in vacuo, redissolved in DCM (40 mL) and washed with water twice. Organic phase was dried over anhydrous sodium sulfate and concentrated in vacuo to give ethyl 2-[m-tolylmethyl(2-pyridylmethyl)amino]-2-oxo-acetate (2.2 g, crude) as a brown oil.

LCMS (ESI): [M+H]⁺m/z: calcd 313.16; found 313.0; Rt=1.183 min.

### Step 3: N′-(m-tolylmethyl)-N′-(2-pyridylmethyl)oxamide

Ethyl 2-[m-tolylmethyl(2-pyridylmethyl)amino]-2-oxo-acetate (2.2 g, 4.58 mmol) was dissolved in MeOH (20 mL) saturated with NH3 (4.58 mmol) and stirred overnight. The RM was concentrated, redissolved in DCM, washed with water, dried over anhydrous sodium sulfate and concentrated in vacuo to give N′-(m-tolylmethyl)-N′-(2-pyridylmethyl)oxamide (1.2 g, crude) as a brown solid.

LCMS (ESI): [M+H]⁺m/z: calcd 284.14; found 284.2; Rt=0.875 min.

### Step 4: N-[4-amino-1-(2-trimethylsilylethoxymethyl)pyrazolo[4,3-c]pyridin-7-yl]-N′-(m-tolylmethyl)-N′-(2-pyridylmethyl)oxamide

Copper (1.68 mg, 26.47 μmol), Copper (I) iodide (50.41 mg, 264.71 μmol, 8.97 μL), cesium carbonate (345.00 mg, 1.06 mmol) were added to a stirred solution of N′-(m-tolylmethyl)-N′-(2-pyridylmethyl)oxamide (300 mg, 529.43 μmol), 7-bromo-1-(2-trimethylsilylethoxymethyl)pyrazolo[4,3-c]pyridin-4-amine (181.75 mg, 529.43 μmol) and rac-(1R,2R)—N1,N2-dimethylcyclohexane-1,2-diamine (146.85 mg, 1.03 mmol) in 1,4-dioxane (5 mL) under Ar atmosphere and stirred at 100° C. for 12 hr in closed vial. The reaction mixture was treated with water, then oily residue on the filter was dissolved in DCM (30 mL), dried over anhydrous sodium sulfate and evaporated in vacuo to give N-[4-amino-1-(2-trimethylsilylethoxymethyl)pyrazolo[4,3-c]pyridin-7-yl]-N′-(m-tolylmethyl)-N′-(2-pyridylmethyl)oxamide (380 mg, crude) as a brown oil.

LCMS (ESI): [M+H]⁺m/z: calcd 546.27; found 546.4; Rt=1.273 min.

### Step 5: The synthesis of N-(4-amino-1H-pyrazolo[4,3-c]pyridin-7-yl)-N′-(m-tolylmethyl)-N′-(2-pyridylmethyl)oxamide

N-[4-amino-1-(2-trimethylsilylethoxymethyl)pyrazolo[4,3-c]pyridin-7-yl]-N′-(m-tolylmethyl)-N′-(2-pyridylmethyl)oxamide (380 mg, 243.72 μmol) was dissolved in Dioxane (4 mL) saturated with HCl (10% by weight). MeOH (4 mL) was added for better solubility. The RM was stirred for 12 hr at 20° C. then concentrated in vacuo. The residue was dissolved in 3 mL of MeOH and subjected to HPLC (Device (Mobile Phase, Column): 2-10 min 45-60% water-methanol+NH₃), then repurified by HPLC (Device (Mobile Phase, Column): 2-10 min 20-50% MeOH+FA flow 30 mL/min) to afford N-(4-amino-1H-pyrazolo[4,3-c]pyridin-7-yl)-N′-(m-tolylmethyl)-N′-(2-pyridylmethyl)oxamide (11.1 mg, 24.05 μmol, 9.87% yield, HCOOH) as a yellow solid.

¹H NMR (600 MHz, dmso) δ 2.21-2.31 (m, 3H), 4.50-4.58 (m, 2H), 4.79-4.91 (m, 2H), 6.62-6.91 (m, 2H), 7.05-7.11 (m, 2H), 7.11-7.26 (m, 2H), 7.26-7.42 (m, 2H), 7.54-7.70 (m, 1H), 7.71-7.82 (m, 1H), 8.14-8.31 (m, 1H), 8.45-8.56 (m, 1H), 9.72-10.63 (m, 1H), 12.67-13.41 (m, 1H).

LCMS (ESI): [M+H]⁺m/z: calcd 416.2; found 416.0; Rt=2.446 min.

### Example 66. Synthesis of N-(4-amino-1H-pyrazolo[4,3-c]pyridin-7-yl)-N′-ethyl-N′-[1-[4-(trifluoromethyl)phenyl]ethyl]oxamide (Compound 58)

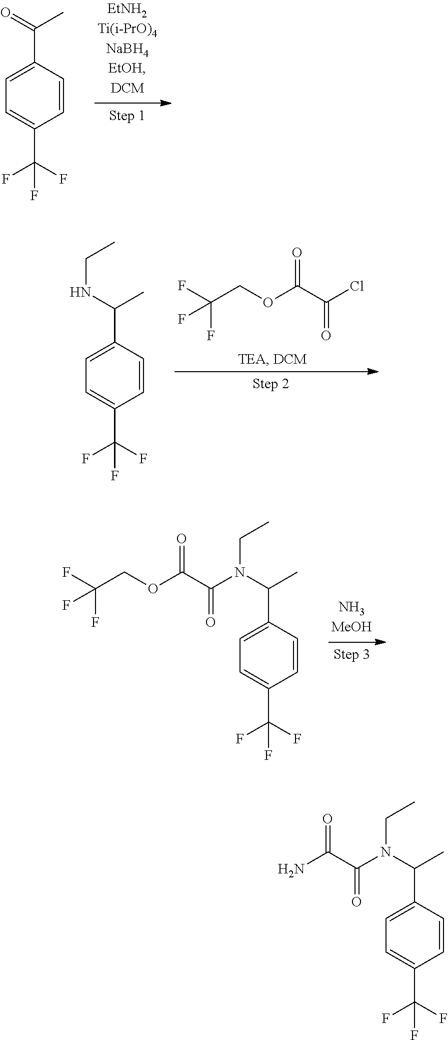

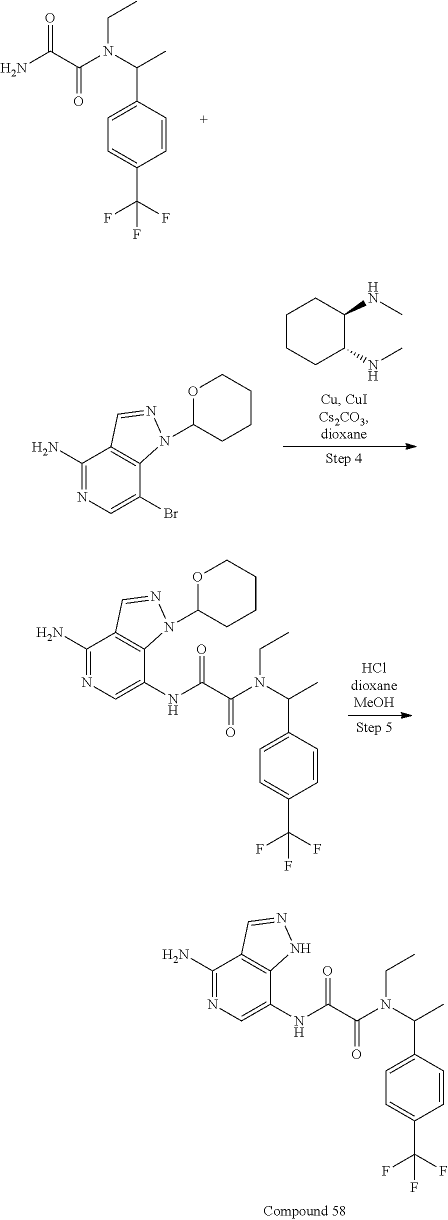

### Step 1: N-ethyl-1-[4-(trifluoromethyl)phenyl]ethanamine

A mixture of the 1-[4-(trifluoromethyl)phenyl]ethanone (187 mg, 993.91 μmol), titanium isopropoxide (423.72 mg, 1.49 mmol, 443.69 μL), ethanamine (121.57 mg, 1.49 mmol, 151.40 μL, HCl) and TEA (150.86 mg, 1.49 mmol, 207.80 μL) in Ethanol (5.06 mL) was stirred under Ar at ambient temperature for 15 hr. Sodium Borohydride (26.32 mg, 695.74 μmol, 24.51 μL) was then added and the resulting mixture was stirred for an additional 8 hr at ambient temperature. The reaction was then quenched by pouring into aqueous ammonia (10 mL), the resulting inorganic precipitate was filtered off, and washed with dichloromethane (10 mL). The organic layer was separated, and the remaining aqueous layer was extracted once with dichloromethane. Organic phase was combined, dried over anhydrous sodium sulfate, filtered and evaporated to obtain N-ethyl-1-[4-(trifluoromethyl)phenyl]ethanamine (187 mg, crude) as a yellow oil.

LCMS (ESI): [M+H]⁺m/z: calcd 218.12; found 218.2; Rt=0.700 min.

### Step 2:2,2,2-trifluoroethyl 2-[ethyl-[1-[4-(trifluoromethyl)phenyl]ethyl]amino]-2-oxo-acetate

2,2,2-trifluoroethyl 2-chloro-2-oxo-acetate (180.39 mg, 946.92 μmol, 122.72 μL) was added dropwise to a solution of N-ethyl-1-[4-(trifluoromethyl)phenyl]ethanamine (187 mg, 860.84 μmol) and triethylamine (130.66 mg, 1.29 mmol, 179.98 μL) in DCM (3.89 mL) at 0° C. After addition was complete, cooling bath was removed and resulting mixture was allowed to warm up to 25° C. and stirred for 16 hr. Then, it was washed with water, dried over anhydrous sodium sulfate and concentrated under reduced pressure, affording 2,2,2-trifluoroethyl 2-[ethyl-[1-[4-(trifluoromethyl)phenyl]ethyl]amino]-2-oxo-acetate (256 mg, crude) as a yellow oil which was used in the next step without further purification.

LCMS (ESI): [M+H]⁺m/z: calcd 372.11; found 372.2; Rt=1.341 min.

### Step 3: N′-ethyl-N′-[1-[4-(trifluoromethyl)phenyl]ethyl]oxamide

A mixture of 2,2,2-trifluoroethyl 2-[ethyl-[1-[4-(trifluoromethyl)phenyl]ethyl]amino]-2-oxo-acetate (256 mg, 689.52 μmol) in Methanol/NH3 (5 mL) was stirred at rt for 15 hr. The solvent was evaporated to obtain N′-ethyl-N′-[1-[4-(trifluoromethyl)phenyl]ethyl]oxamide (142 mg, crude) as a yellow gum.

LCMS (ESI): [M−H]⁻m/z: calcd 287.1; found 287.2; Rt=1.153 min.

### Step 4: N-(4-amino-1-tetrahydropyran-2-yl-pyrazolo[4,3-c]pyridin-7-yl)-N′-ethyl-N′-[1-[4-(trifluoromethyl)phenyl]ethyl]oxamide

Copper (1.57 mg, 24.63 μmol), Copper (I) iodide (46.91 mg, 246.30 μmol, 8.35 μL), cesium carbonate (240.75 mg, 738.90 μmol) was added to a stirred solution of N′-ethyl-N′-[1-[4-(trifluoromethyl)phenyl]ethyl]oxamide (142 mg, 492.60 μmol), 7-bromo-1-tetrahydropyran-2-yl-pyrazolo[4,3-c]pyridin-4-amine (146.38 mg, 492.60 μmol), rac-(1R,2R)—N1,N2-dimethylcyclohexane-1,2-diamine (35.03 mg, 246.30 μmol) in 1,4-dioxane (4 mL) under Ar atmosphere and stirred at 90° C. for 48 hr in closed vial. The reaction mixture was cooled and filtered. The filtercake was washed with MeOH (10 mL) and the filtrate was concentrated in vacuo to obtain N-(4-amino-1-tetrahydropyran-2-yl-pyrazolo[4,3-c]pyridin-7-yl)-N′-ethyl-N′-[1-[4-(trifluoromethyl)phenyl]ethyl]oxamide (215 mg, crude) as a brown solid which was used in the next step without purification.

LCMS (ESI): [M+H]⁺m/z: calcd 505.22; found 505.2; Rt=1.053 min.

### Step 5: The synthesis of N-(4-amino-1H-pyrazolo[4,3-c]pyridin-7-yl)-N′-ethyl-N′-[1-[4-(trifluoromethyl)phenyl]ethyl]oxamide

N-(4-amino-1-tetrahydropyran-2-yl-pyrazolo[4,3-c]pyridin-7-yl)-N′-ethyl-N′-[1-[4-(trifluoromethyl)phenyl]ethyl]oxamide (215 mg, 426.16 μmol) was dissolved in Methanol (2 mL) and Dioxane/HCl (2 mL) and resulting mixture was stirred overnight. The reaction mixture was concentrated in vacuo. The residue was purified by HPLC (0-2-10 min 23-30-45 H₂O/MeOH/0.1NH4OH flow 30 mL/min ((loading pump 4 mL MeOH) target mass 425; column: XBridge BEH C18 100×19 mm, 5 microM) to obtain N-(4-amino-1H-pyrazolo[4,3-c]pyridin-7-yl)-N′-ethyl-N′-[1-[4-(trifluoromethyl)phenyl]ethyl]oxamide (17.7 mg, 42.10 μmol, 9.88% yield) as a light-yellow solid.

¹H NMR (600 MHz, DMSO-d₆) δ 0.87-1.08 (m, 3H), 1.34-1.72 (m, 3H), 2.95-3.27 (m, 1H), 3.34-3.59 (m, 1H), 5.00-5.65 (m, 1H), 6.60-6.98 (m, 2H), 7.34-7.80 (m, 5H), 8.12-8.33 (m, 1H), 9.44-10.68 (m, 1H), 12.59-13.34 (m, 1H).

LCMS (ESI): [M+H]⁺m/z: calcd 421.18; found 421.2; Rt=0.853 min.

### Example 67. Synthesis of N-(4-amino-2H-pyrazolo[4,3-c]pyridin-7-yl)-N′-ethyl-N′-[[2-fluoro-4-(trifluoromethyl)phenyl]methyl]oxamide (Compound 72)

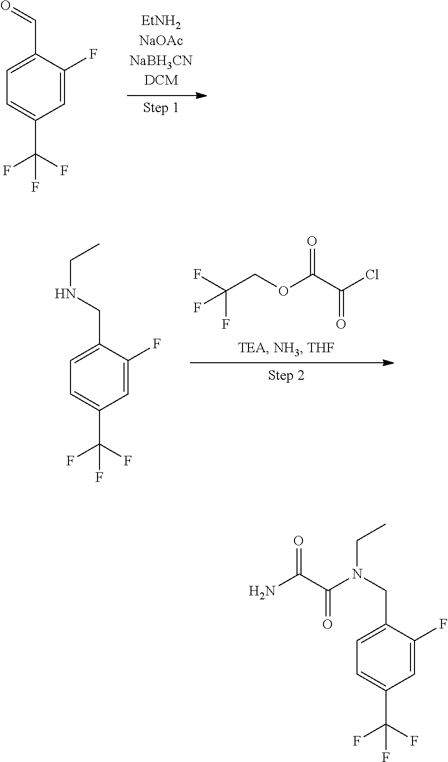 [SMILES: O=Cc1ccc(C(F)(F)F)cc1F ; CCNCc1ccc(C(F)(F)F)cc1F ; O=C(Cl)C(=O)OCC(F)(F)F ; CCN(Cc1ccc(C(F)(F)F)cc1F)C(=O)C(N)=O]

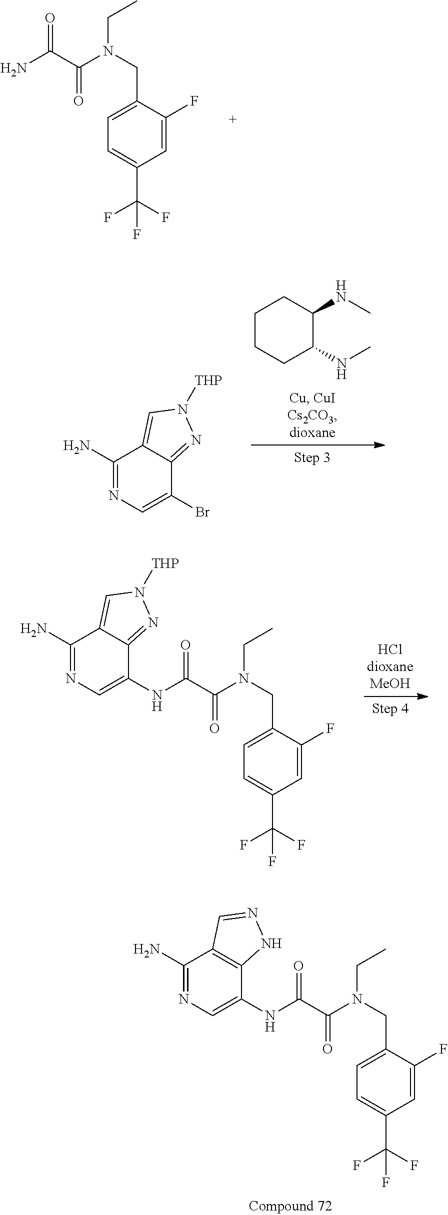

### Step 1: N-[[2-fluoro-4-(trifluoromethyl)phenyl]methyl]ethanamine

To a solution of ethanamine (1.02 g, 12.49 mmol, 1.27 mL, HCl) and Sodium acetate (1.33 g, 16.24 mmol, 871.87 μL) in Methanol (50 mL), 2-fluoro-4-(trifluoromethyl)benzaldehyde (1.2 g, 6.25 mmol) was added. After 30 min, Sodium cyanoborohydride (785.05 mg, 12.49 mmol, 1.08 mL) was added and the resulting mixture was stirred at 20° C. for 18 hr. The solvent was evaporated in vacuo and the residue was taken up with K₂CO₃ (25 mL, sat. aq.) and extracted with DCM (2×25 mL). The combined organic layers were dried over anhydrous sodium sulfate and evaporated in vacuo to obtain N-[[2-fluoro-4-(trifluoromethyl)phenyl]methyl]ethanamine (1.35 g, 6.10 mmol, 97.71% yield) as a yellow oil.

LCMS (ESI): [M+H]⁺m/z: calcd 222.09; found 222.0; Rt=0.554 min.

### Step 2: N′-ethyl-N′-[[2-fluoro-4-(trifluoromethyl)phenyl]methyl]oxamide

To a solution of N-[[2-fluoro-4-(trifluoromethyl)phenyl]methyl]ethanamine (1.37 g, 6.19 mmol) and TEA (1.25 g, 12.39 mmol, 1.73 mL) in THF (30 mL) was added 2,2,2-trifluoroethyl 2-chloro-2-oxo-acetate (1.77 g, 9.29 mmol) dropwise at 0° C. under argon. The reaction mixture was then stirred for 12 hr at r.t. Then ammonia (292.20 mg, 17.16 mmol) was bubbled through for 10 min at 0° C. The reaction mixture was then stirred for 12 hr at r.t. The reaction mixture was filtered off and the filtrate was evaporated in vacuo to give N′-ethyl-N′-[[2-fluoro-4-(trifluoromethyl)phenyl]methyl]oxamide (1.5 g, 5.13 mmol, 82.87% yield) as a brown oil.

LCMS (ESI): [M+H]⁺m/z: calcd 293.09; found 293.0; Rt=1.236 min.

### Step 3: N-(4-amino-2-tetrahydropyran-2-yl-pyrazolo[4,3-c]pyridin-7-yl)-N′-ethyl-N′-[[2-fluoro-4-(trifluoromethyl)phenyl]methyl]oxamide

N′-ethyl-N′-[2-fluoro-4-(trifluoromethyl)phenyl]methyl]oxamide (200 mg, 684.39 μmol), 7-bromo-2-tetrahydropyran-2-yl-pyrazolo[4,3-c]pyridin-4-amine (305.05 mg, 1.03 mmol), Cu(4.61 mg, 72.55 μmol), CuI (39.10 mg, 205.32 μmol, 6.96 μL), cesium carbonate (334.48 mg, 1.03 mmol, 146.06 μL) and (1R,2R)—N1,N2-dimethylcyclohexane-1,2-diamine (29.20 mg, 205.32 μmol) were mixed in dioxane (10 mL), purged with Ar for 2 minutes and then heated in the sealed tube at 100° C. for 18 hr. Final mixture was filtered and the filtrate was evaporated in vacuo to afford N-(4-amino-2-tetrahydropyran-2-yl-pyrazolo[4,3-c]pyridin-7-yl)-N′-ethyl-N′-[2-fluoro-4-(trifluoromethyl)phenyl]methyl]oxamide (0.5 g, crude) as a brown solid which was used in the next step without further purification.

LCMS (ESI): [M+H]⁺m/z: calcd 509.19; found 509.2; Rt=0.956 min.

### Step 4: The synthesis of N-(4-amino-2H-pyrazolo[4,3-c]pyridin-7-yl)-N′-ethyl-N′-[[2-fluoro-4-(trifluoromethyl)phenyl]methyl]oxamide

To N-(4-amino-2-tetrahydropyran-2-yl-pyrazolo[4,3-c]pyridin-7-yl)-N′-ethyl-N′-[[2-fluoro-4-(trifluoromethyl)phenyl]methyl]oxamide (0.5 g, 983.35 μmol) in methanol (15 mL) was added Hydrogen chloride solution 3.0M in dioxane (5.38 g, 14.75 mmol, 5.12 mL, 10% purity) at 21° C. The resulting mixture was left to stir for 18 hr. The resulting mixture was evaporated to dryness. The residue was purified by RP-HPLC (column: XBridge C18 OBD 100×30 mm 5 μm; 30-80% 0-5 min H₂O/MeOH/0.1% NH₄OH, flow: 30 ml/min) to give N-(4-amino-2H-pyrazolo[4,3-c]pyridin-7-yl)-N′-ethyl-N′-[2-fluoro-4-(trifluoromethyl)phenyl]methyl]oxamide (40 mg, 94.26 μmol, 9.59% yield) as a yellow solid.

¹H NMR (600 MHz, dmso) 8 1.04-1.25 (m, 3H), 3.33-3.42 (m, 1H), 3.57 (q, 1H), 4.35-5.05 (m, 2H), 6.56-7.23 (m, 2H), 7.47-7.79 (m, 4H), 8.17 (d, 1H), 9.66-10.58 (m, 1H), 12.63-13.42 (m, 1H).

LCMS (ESI): [M+H]⁺m/z: calcd 425.15; found 425.2; Rt=2.273 min.

### Example 68. Synthesis of (R)—N¹-(4-amino-1H-pyrazolo[4,3-c]pyridin-7-yl)-N2-methyl-N2-(1-(2-methyl-4-(trifluoromethyl)phenyl)ethyl)oxalamide (Compound 56) & (S)-N1-(4-amino-1H-pyrazolo[4,3-c]pyridin-7-yl)-N2-methyl-N2-(1-(2-methyl-4-(trifluoromethyl)phenyl)ethyl)oxalamide (Compound 63)

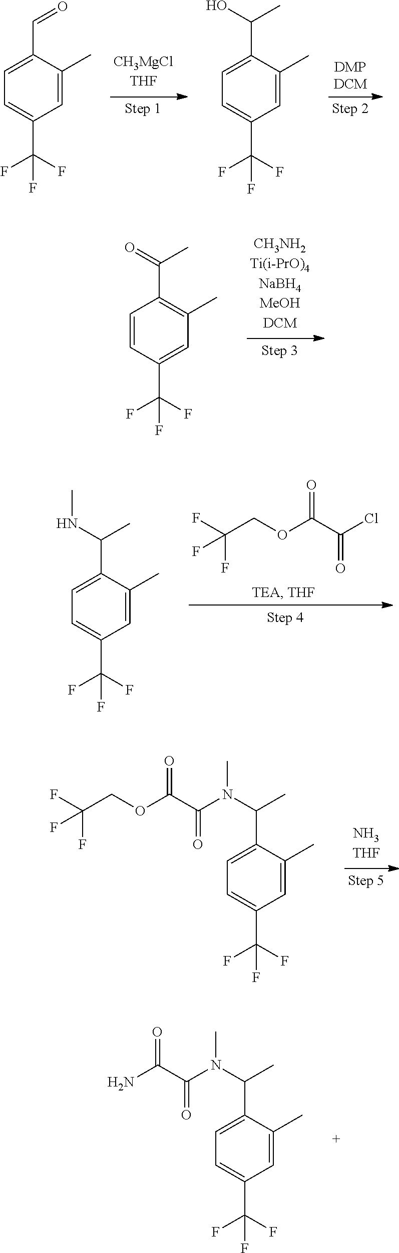

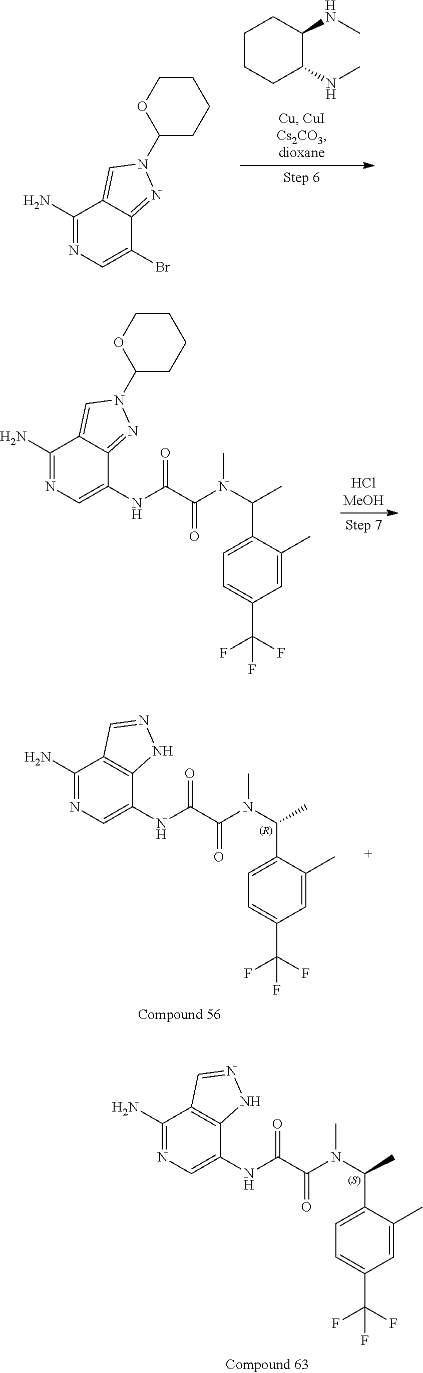

### Step 1:1-[2-methyl-4-(trifluoromethyl)phenyl]ethanol

Chloro(methyl) magnesium (1.94 g, 25.91 mmol) was added dropwise to a stirred solution of 2-methyl-4-(trifluoromethyl)benzaldehyde (3.25 g, 17.27 mmol) in THF (60 mL) at 0° C., slowly allowed to 20° C. and stirred for 2 hr. Water (5 mL) was added, RM, stirred for 10 min and filtered. Filtrate was concentrated in vacuo to give 1-[2-methyl-4-(trifluoromethyl)phenyl]ethanol (3.3 g, 16.16 mmol, 93.56% yield) as a white solid.

### Step 2:1-[2-methyl-4-(trifluoromethyl)phenyl]ethanone

To a solution of 1-[2-methyl-4-(trifluoromethyl)phenyl]ethanol (3.3 g, 16.16 mmol) in DCM (75 mL) was added Dess-Martin Periodinane (8.23 g, 19.39 mmol) in one portion. The resulting mixture was stirred 2 hr at rt. RM was concentrated, residue was triturated with MTBE (200 mL), filtered, filtrate was washed with NaHCO₃ (20 mL, 10% solutions), dried over anhydrous sodium sulfate and concentrated in vacuo to give 1-[2-methyl-4-(trifluoromethyl)phenyl]ethanone (1.5 g, 7.42 mmol, 45.91% yield) as a light-yellow solid.

### Step 3: N-methyl-1-[2-methyl-4-(trifluoromethyl)phenyl]ethanamine

A mixture of the 1-[2-methyl-4-(trifluoromethyl)phenyl]ethanone (0.5 g, 2.47 mmol), titanium isopropoxide (843.47 mg, 2.97 mmol, 883.21 μL), methanamine (2.50 g, 8.06 mmol, 2.79 mL) in DCM (14.72 mL) was stirred under Ar at ambient temperature for 8 hr. Sodium Borohydride (93.56 mg, 2.47 mmol, 87.11 μL) was added and the resulting mixture was stirred for an additional 1 hr at ambient temperature. Sodium hydroxide (5 mL, 20%) was added to a RM and stirred for 10 min, filtered, DCM layer separated, dried over anhydrous sodium sulfate, concentrated in vacuo to give crude N-methyl-1-[2-methyl-4-(trifluoromethyl)phenyl]ethanamine (0.4 g, 1.84 mmol, 74.45% yield) as a light-yellow oil.

LCMS (ESI): [M+H]⁺m/z: calcd 218.12; found 218.0; Rt=0.956 min.

### Step 4:2,2,2-trifluoroethyl 2-(methyl(1-(2-methyl-4-(trifluoromethyl)phenyl)ethyl)amino)-2-oxoacetate

2,2,2-trifluoroethyl 2-chloro-2-oxo-acetate (385.87 mg, 2.03 mmol, 262.50 μL) was added dropwise to a stirred solution of N-methyl-1-[2-methyl-4-(trifluoromethyl)phenyl]ethanamine (0.4 g, 1.84 mmol) and TEA (242.22 mg, 2.39 mmol, 333.64 μL) in THF (15.08 mL) at 25° C., stirred for 1 hr at 25° C. The reaction mixture was used in the next step directly.

### Step 5: N′-methyl-N′-[1-[2-methyl-4-(trifluoromethyl)phenyl]ethyl]oxamide

Ammonia (541.13 mg, 31.77 mmol) was bubbled trough a reaction mixture from previous step at 20° C., stirred for 1 hr at 20° C. The reaction mixture was filtered, solid washed with THE (2×5 mL), filtrate was concentrated in vacuo to give N′-methyl-N′-[1-[2-methyl-4-(trifluoromethyl)phenyl]ethyl]oxamide (0.5 g, 1.73 mmol, 94.70% yield) as a brown gum.

### Step 6: N1-(4-amino-2-(tetrahydro-2H-pyran-2-yl)-2H-pyrazolo[4,3-c]pyridin-7-yl)-N2-methyl-N2-(1-(2-methyl-4-(trifluoromethyl)phenyl)ethyl)oxalamide

Copper (1.32 mg, 20.81 μmol), Copper (I) iodide (39.64 mg, 208.14 μmol, 7.05 L), cesium carbonate (203.45 mg, 624.43 μmol) was added to a stirred solution of N′-methyl-N′-[1-[2-methyl-4-(trifluoromethyl)phenyl]ethyl]oxamide (120 mg, 416.28 μmol), 7-bromo-2-tetrahydropyran-2-yl-pyrazolo[4,3-c]pyridin-4-amine (123.70 mg, 416.28 μmol), rac-(1R,2R)—N1,N2-dimethylcyclohexane-1,2-diamine (29.61 mg, 208.14 μmol) in 1,4-dioxane (7 mL) under Ar atmosphere and stirred at 110° C. for 48 hr in closed vial. The reaction mixture was cooled and filtered. The filtercake was washed with DCM (3 ml) and filtrate was used in the next step directly.

LCMS (ESI): [M+H]⁺m/z: calcd 505.21; found 505.2; Rt=1.063 min.

### Step 7: The synthesis of (R)—N¹-(4-amino-1H-pyrazolo[4,3-c]pyridin-7-yl)-N2-methyl-N2-(1-(2-methyl-4-(trifluoromethyl)phenyl)ethyl)oxalamide & (S)-N1-(4-amino-1H-pyrazolo[4,3-c]pyridin-7-yl)-N2-methyl-N2-(1-(2-methyl-4-(trifluoromethyl)phenyl)ethyl)oxalamide

Hydrogen chloride (338.08 mg, 7.88 mmol, 277.11 μL, 85% purity) was added to a solution of N-(4-amino-2-tetrahydropyran-2-yl-pyrazolo[4,3-c]pyridin-7-yl)-N′-methyl-N′-[1-[2-methyl-4-(trifluoromethyl)phenyl]ethyl]oxamide (0.21 g, 416.25 μmol) in Methanol (2.04 mL) and stirred at 20° C. for 1 hr. Volatiles was evaporated in vacuo and residue submitted to HPLC (column: XBridge C18 100×19 mm, 5 μm; 30-80% 0-5 min H₂O/MeOH/0.1% NH₄OH, flow: 30 ml/min, flow rate: 30 ml/min), then pure racemic product was submitted to chiral HPLC (Column: CHIRALPAK AD-H (250×20 mm, 5 μm)—V; Mobile Phase: Hexane (0.1% DEA): MeOH: IPA, 60:20:20; Injection Volume: 900 μl; flow rate: 12 ml/min) to give (R)—N¹-(4-amino-1H-pyrazolo[4,3-c]pyridin-7-yl)-N2-methyl-N2-(1-(2-methyl-4-(trifluoromethyl)phenyl)ethyl)oxalamide (13 mg, 30.92 μmol, 7.43% yield, Rt=9.588 min) and(S)-N1-(4-amino-1H-pyrazolo[4,3-c]pyridin-7-yl)-N2-methyl-N2-(1-(2-methyl-4-(trifluoromethyl)phenyl)ethyl)oxalamide (11 mg, 26.17 μmol, 6.29% yield, RT=14.012 min) as light-brown solids.

The absolute stereochemistry of the two compounds was independently confirmed.

### Compound 63:

LCMS (ESI): [M+H]⁺m/z: calcd 420.18; found 421.2; Rt=2.570 min.

### Compound 56:

LCMS (ESI): [M+H]⁺m/z: calcd 420.18; found 421.2; Rt=2.599 min.

### Example 69. Synthesis of(S)-N1-(4-amino-1H-pyrazolo[4,3-c]pyridin-7-yl)-N2-ethyl-N2-(1-(2-methyl-4-(perfluoroethyl)phenyl)ethyl)oxalamide (Compound 69) & (R)—N¹-(4-amino-1H-pyrazolo[4,3-c]pyridin-7-yl)-N2-ethyl-N2-(1-(2-methyl-4-(perfluoroethyl)phenyl)ethyl)oxalamide (Compound 59)

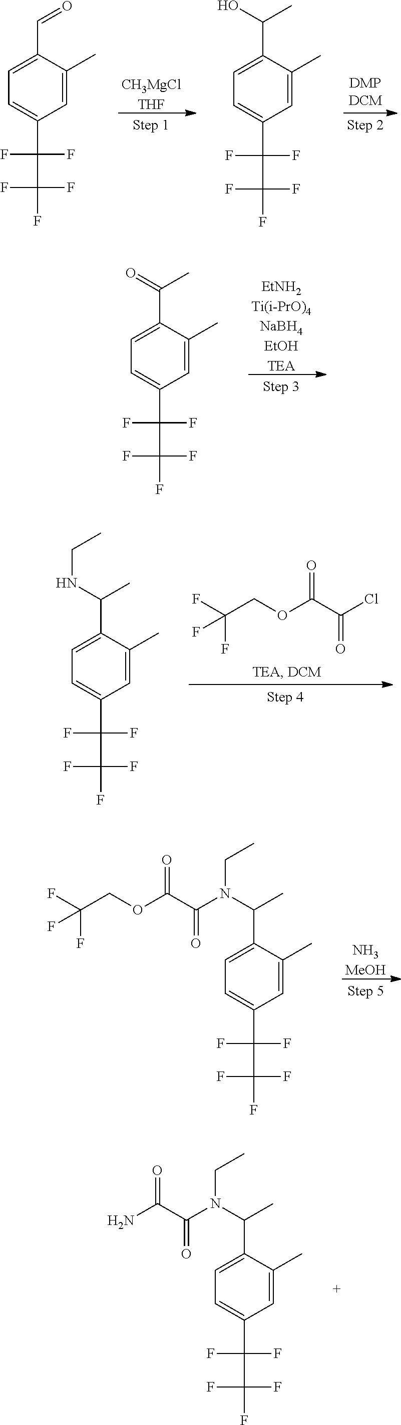

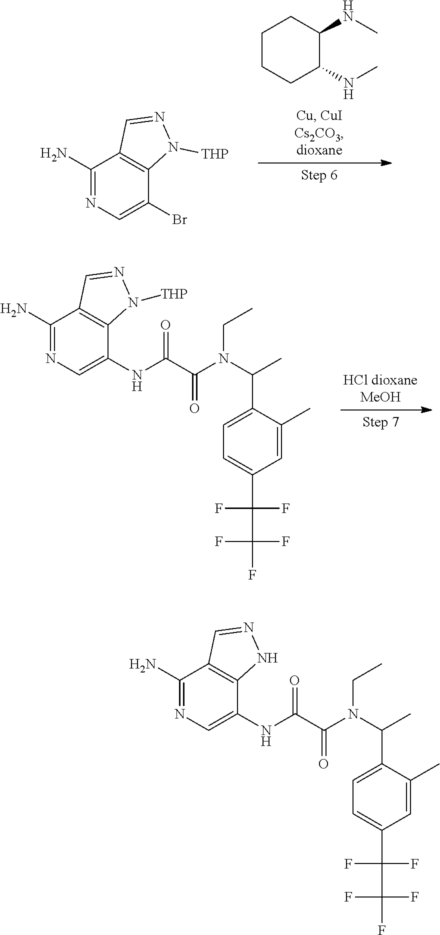

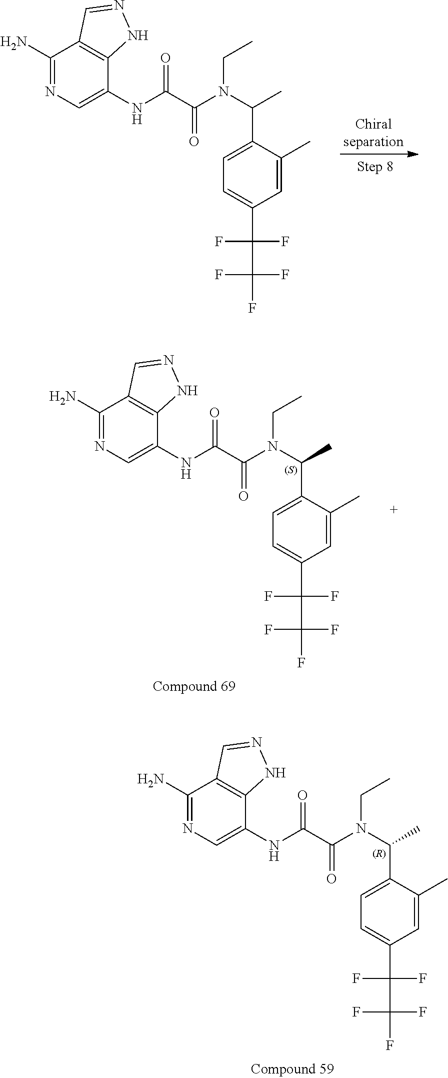

### Step 1:1-[2-methyl-4-(1,1,2,2,2-pentafluoroethyl)phenyl]ethanol

Chloro(methyl) magnesium (392.56 mg, 5.25 mmol) was added to the solution of 2-methyl-4-(1,1,2,2,2-pentafluoroethyl)benzaldehyde (1.25 g, 5.25 mmol) in THF (25 mL) maintaining the internal temperature below 25° C. The resulting reaction mixture was allowed to stir at room temperature for 12 hr, then quenched with saturated NH₄Cl solution (15 mL) and extracted with EtOAc (2×15 mL). The combined organic layers were dried over anhydrous sodium sulfate and filtered. The filtrate was concentrated under reduced pressure to obtain product 1-[2-methyl-4-(1,1,2,2,2-pentafluoroethyl)phenyl]ethanol (1.05 g, crude) as a light-yellow solid which was used in the next step reaction without further purification.

LCMS (ESI): [M−H]⁻m/z: calcd 253.06; found 253.0; Rt=1.313 min.

### Step 2: 1~ [2-methyl-4-(1,1,2,2,2-pentafluoroethyl)phenyl]ethanone

To a solution of 1-[2-methyl-4-(1,1,2,2,2-pentafluoroethyl)phenyl]ethanol (1.05 g, 4.13 mmol) in DCM (30 mL) was added Dess-Martin Periodinane (2.10 g, 4.96 mmol) in one portion. The resulting mixture was stirred 2 hr at rt. The reaction mixture was poured into a solution containing Na₂S₂O₃ and Na₂CO₃ (6 g: 3 g in 50 mL of H₂O), stirred for 30 min, DCM was dried and evaporated. 1-[2-methyl-4-(1,1,2,2,2-pentafluoroethyl)phenyl]ethanone (0.87 g, crude) was obtained as a light-yellow oil.

LCMS (ESI): [M+H]⁺m/z: calcd 253.07; found 253.0; Rt=1.445 min.

### Step 3: N-ethyl-1-[2-methyl-4-(1,1,2,2,2-pentafluoroethyl)phenyl]ethanamine

A mixture of the 1-[2-methyl-4-(1,1,2,2,2-pentafluoroethyl)phenyl]ethanone (0.76 g, 3.01 mmol), titanium isopropoxide (1.28 g, 4.52 mmol, 1.35 mL), ethanamine (368.63 mg, 4.52 mmol, 459.06 μL, HCl) and TEA (457.44 mg, 4.52 mmol, 630.08 μL) in Ethanol (79.90 mL) was stirred under Ar at ambient temperature for 12 hr. Sodium Borohydride (114.01 mg, 3.01 mmol, 106.15 μL) was then added and the resulting mixture was stirred for an additional 1 hr at ambient temperature. The reaction was then quenched by pouring into aqueous ammonia (10 mL), the resulting inorganic precipitate was filtered off, and washed with dichloromethane (10 mL). The organic layer was separated, and the remaining aqueous layer was extracted once with dichloromethane. Organic phase was combined, dried over anhydrous sodium sulfate, filtered and the filtrate was evaporated. The resulting crude product was poured into water (20 mL) and NaHSO₄ was added to pH=3. The resulting mixture was washed with DCM (2×20 mL). Water was separated and alkalized with K₂CO₃ to pH=10. The solution was extracted with DCM (2×20 mL). Organic phase was separated, dried over anhydrous sodium sulfate and filtered. The filtrate was evaporated to obtain N-ethyl-1-[2-methyl-4-(1,1,2,2,2-pentafluoroethyl)phenyl]ethanamine (140 mg, crude) as a light-yellow oil.

LCMS (ESI): [M+H]⁺m/z: calcd 282.13; found 282.2; Rt=0.800 min.

### Step 4:2,2,2-trifluoroethyl 2-[ethyl-[1-[2-methyl-4-(1,1,2,2,2-pentafluoroethyl)phenyl]ethyl]amino]-2-oxo-acetate

To a stirred solution of N-ethyl-1-[2-methyl-4-(1,1,2,2,2-pentafluoroethyl)phenyl]ethanamine (140 mg, 497.75 μmol) in Dichloromethane (2.92 mL) were added triethylamine (75.55 mg, 746.63 μmol, 104.07 μL) respectively at room temperature. The resulting reaction mixture was cooled to 0° C. Then 2,2,2-trifluoroethyl 2-chloro-2-oxo-acetate (113.79 mg, 597.30 μmol, 77.41 μL) was added dropwise. The reaction was stirred extra 30 minutes at 0° C., then allowed warmed to room temperature and stirred 12 hr. Upon completion, the reaction mixture was washed with water (2×20 mL). Organic phase was then dried over anhydrous sodium sulfate and filtered. The filtrate was concentrated under reduced pressure to obtain 2,2,2-trifluoroethyl 2-[ethyl-[1-[2-methyl-4-(1,1,2,2,2-pentafluoroethyl)phenyl]ethyl]amino]-2-oxo-acetate (164 mg, crude) as a brown gum which was used in the next step without further purification.

LCMS (ESI): [M+H]⁺m/z: calcd 436.12; found 436.0; Rt=1.608 min.

### Step 5: N′-ethyl-N′-[1-[2-methyl-4-(1,1,2,2,2-pentafluoroethyl)phenyl]ethyl]oxamide

A solution of 2,2,2-trifluoroethyl 2-[ethyl-[1-[2-methyl-4-(1,1,2,2,2-pentafluoroethyl)phenyl]ethyl]amino]-2-oxo-acetate (164 mg, 376.74 μmol) in Methanol/NH3 (7N, 2 mL) was stirred at 25° C. for 12 hr. The solvent was evaporated to obtain N′-ethyl-N′-[1-[2-methyl-4-(1,1,2,2,2-pentafluoroethyl)phenyl]ethyl]oxamide (132 g, crude) as a brown gum.

### Step 6: N-(4-amino-1-tetrahydropyran-2-yl-pyrazolo[4,3-c]pyridin-7-yl)-N′-ethyl-N′-[1-[2-methyl-4-(1,1,2,2,2-pentafluoroethyl)phenyl]ethyl]oxamide

Copper (1.19 mg, 18.73 μmol), copper (I) iodide (35.68 mg, 187.34 μmol, 6.35 μL), cesium carbonate (183.12 mg, 562.02 μmol) was added to a stirred solution of N′-ethyl-N′-[1-[2-methyl-4-(1,1,2,2,2-pentafluoroethyl)phenyl]ethyl]oxamide (132 mg, 374.68 μmol), 7-bromo-1-tetrahydropyran-2-yl-pyrazolo[4,3-c]pyridin-4-amine (111.34 mg, 374.68 μmol), rac-(1R,2R)—N1,N2-dimethylcyclohexane-1,2-diamine (26.65 mg, 187.34 μmol) in 1,4-dioxane (4 mL) under Ar atmosphere and stirred at 90° C. for 48 hr in closed vial. The reaction mixture was cooled and filtered. The filtercake was washed with MeOH (10 mL) and the filtrate was concentrated in vacuo to obtain N-(4-amino-1-tetrahydropyran-2-yl-pyrazolo[4,3-c]pyridin-7-yl)-N′-ethyl-N′-[1-[2-methyl-4-(1,1,2,2,2-pentafluoroethyl)phenyl]ethyl]oxamide (247 mg, crude) as a brown solid which was used in the next step without further purification.

LCMS (ESI): [M+H]⁺m/z: calcd 569.23; found 569.2; Rt=1.384 min.

### Step 7: N-(4-amino-1H-pyrazolo[4,3-c]pyridin-7-yl)-N′-ethyl-N′-[1-[2-methyl-4-(1,1,2,2,2-pentafluoroethyl)phenyl]ethyl]oxamide

N-(4-amino-1-tetrahydropyran-2-yl-pyrazolo[4,3-c]pyridin-7-yl)-N′-ethyl-N′-[1-[2-methyl-4-(1,1,2,2,2-pentafluoroethyl)phenyl]ethyl]oxamide (247 mg, 434.45 μmol) was dissolved in Dioxane/HCl (2 mL) and Methanol (2 mL). The resulting solution was stirred at rt for 12 hr. Solvents were evaporated. The resulting product was purified by HPLC (0-2-8 min Jul. 15, 1965% H₂O/ACN/0.1% FA flow 30 ml/min (loading pump 4 ml/min ACN), target mass 484; column: Chromatorex C18 SMB100-5T 100×19 mm, 5 μM) to obtain N-(4-amino-1H-pyrazolo[4,3-c]pyridin-7-yl)-N′-ethyl-N′-[1-[2-methyl-4-(1,1,2,2,2-pentafluoroethyl)phenyl]ethyl]oxamide (14.5 mg, crude, HCOOH) as a light-yellow solid.

LCMS (ESI): [M+H]⁺m/z: calcd 485.2; found 485.0; Rt=1.171 min.

### Step 8: The synthesis of (R)—N¹-(4-amino-1H-pyrazolo[4,3-c]pyridin-7-yl)-N2-ethyl-N2-(1-(2-methyl-4-(perfluoroethyl)phenyl)ethyl)oxalamide & (S)-N1-(4-amino-1H-pyrazolo[4,3-c]pyridin-7-yl)-N2-ethyl-N2-(1-(2-methyl-4-(perfluoroethyl)phenyl)ethyl)oxalamide

N-(4-amino-1H-pyrazolo[4,3-c]pyridin-7-yl)-N′-ethyl-N′-[1-[2-methyl-4-(1,1,2,2,2-pentafluoroethyl)phenyl]ethyl]oxamide (14.5 mg, 29.93 μmol) was chirally separated (Column: CHIRALPAK IC (250×20 mm, 5 μm)-II; Mobile phase: Hexane (0.1% DEA): IPA: MeOH, 60:20:20; flow rate: 12 mL/min) to obtain(S)-N1-(4-amino-1H-pyrazolo[4,3-c]pyridin-7-yl)-N2-ethyl-N2-(1-(2-methyl-4-(perfluoroethyl)phenyl)ethyl)oxalamide (4.51 mg, 9.31 μmol, 31.10% yield, Rt-9.046 min) and (R)—N¹-(4-amino-1H-pyrazolo[4,3-c]pyridin-7-yl)-N2-ethyl-N2-(1-(2-methyl-4-(perfluoroethyl)phenyl)ethyl)oxalamide (4.8 mg, 9.91 μmol, 33.10% yield, Rt=11.357 min) as light-yellow solids.

The absolute stereochemistry of the two compounds was independently confirmed.

### Compound 69:

¹H NMR (600 MHz, dmso) 8 0.49-0.74 (m, 3H), 1.44-1.72 (m, 3H), 2.32-2.43 (m, 3H), 2.88-3.26 (m, 2H), 5.35-5.87 (m, 1H), 6.57-6.85 (m, 2H), 7.36-7.60 (m, 2H), 7.59-7.69 (m, 1H), 7.70-7.81 (m, 1H), 8.11-8.21 (m, 1H), 9.54-10.59 (m, 1H), 12.62-13.28 (m, 1H).

LCMS (ESI): [M+H]⁺m/z: calcd 484.2; found 485.2; Rt=2.752 min.

### Compound 59:

¹H NMR (600 MHz, dmso) 8 0.49-0.75 (m, 3H), 1.47-1.73 (m, 3H), 2.32-2.43 (m, 3H), 2.83-3.17 (m, 2H), 5.35-5.87 (m, 1H), 6.56-6.85 (m, 2H), 7.39-7.69 (m, 3H), 7.70-7.83 (m, 1H), 8.02-8.23 (m, 1H), 9.48-10.62 (m, 1H), 12.52-13.36 (m, 1H).

LCMS (ESI): [M+H]⁺m/z: calcd 484.2; found 485.2; Rt=2.757 min.

### Example 70. Synthesis of (R)—N¹-(4-amino-1H-pyrazolo[4,3-c]pyridin-7-yl)-N2-ethyl-N2-(1-(2-methyl-4-(trifluoromethyl)phenyl)ethyl)oxalamide (Compound 70) & (S)-N1-(4-amino-1H-pyrazolo[4,3-c]pyridin-7-yl)-N2-ethyl-N2-(1-(2-methyl-4-(trifluoromethyl)phenyl)ethyl)oxalamide (Compound 57)

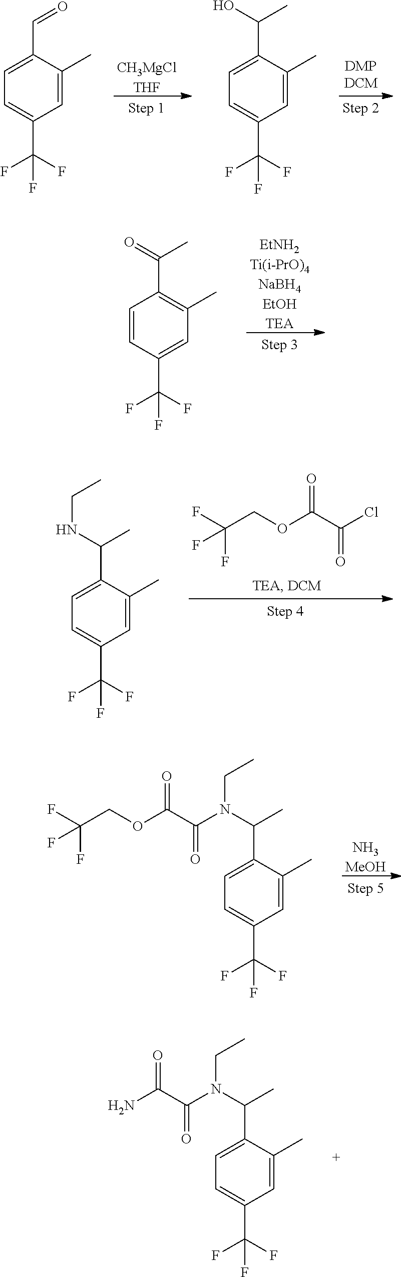

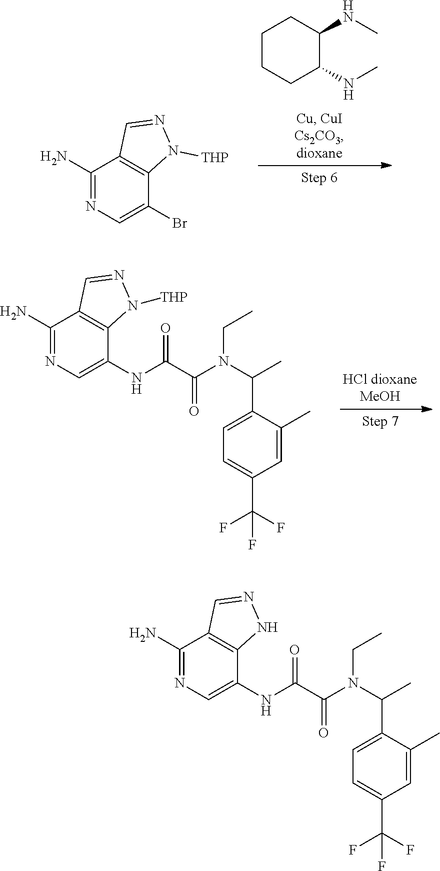

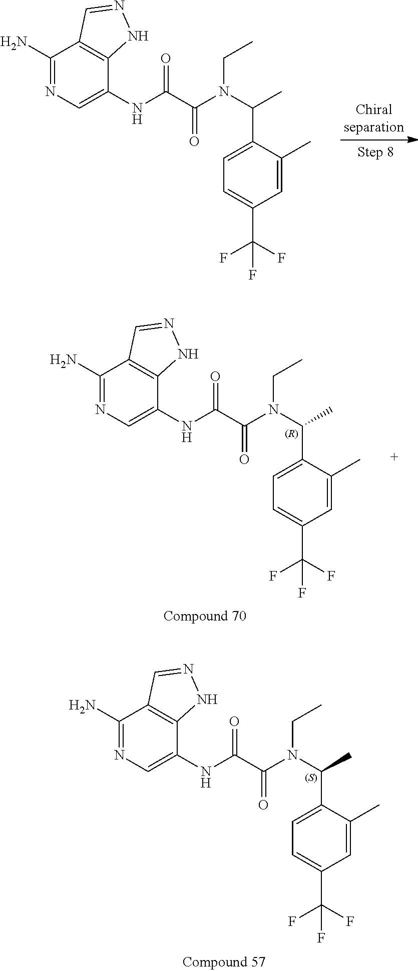

### Step 1:1-[2-methyl-4-(trifluoromethyl)phenyl]ethanol

Chloro(methyl) magnesium (447.21 mg, 5.98 mmol, 1.99 mL) was added to the solution of 2-methyl-4-(trifluoromethyl)benzaldehyde (750 mg, 3.99 mmol) in THF (15 mL) maintaining the internal temperature below 25° C. The resulting reaction mixture was allowed to stir at room temperature for 12 hr, then quenched with saturated NH₄Cl solution (25 mL) and extracted with EtOAc (2×25 mL). The combined organic layers were dried over anhydrous sodium sulfate and filtered. The filtrate was concentrated under reduced pressure to obtain product 1-[2-methyl-4-(trifluoromethyl)phenyl]ethanol (0.72 g, crude) as a light-yellow oil which was used in the next step without further purification.

LCMS (ESI): [M−H]⁻m/z: calcd 202.07; found 203.0; Rt=1.196 min.

### Step 2:1-[2-methyl-4-(trifluoromethyl)phenyl]ethanone

To a solution of 1-[2-methyl-4-(trifluoromethyl)phenyl]ethanol (0.75 g, 3.67 mmol) in DCM (30 mL) was added Dess-Martin Periodinane (1.87 g, 4.41 mmol) in one portion. The resulting mixture was stirred for 2 hr at rt. The reaction mixture was poured into a solution containing Na₂S₂O₃ and Na₂CO₃ (2 g/1 g in 10 mL of H₂O), stirred for 30 min, DCM was dried and evaporated. 1-[2-methyl-4-(trifluoromethyl)phenyl]ethanone (560 mg, crude) was obtained as a light-yellow oil.

LCMS (ESI): [M+H]⁺m/z: calcd 202.07; found 202.8; Rt=1.364 min.

### Step 3: N-ethyl-1-[2-methyl-4-(trifluoromethyl)phenyl]ethanamine

A mixture of the 1-[2-methyl-4-(trifluoromethyl)phenyl]ethanone (0.43 g, 2.13 mmol), titanium isopropoxide (906.73 mg, 3.19 mmol, 949.45 μL), ethanamine (260.15 mg, 3.19 mmol, 323.98 μL, HCl) and TEA (322.83 mg, 3.19 mmol, 444.67 μL) in Ethanol (30 mL) was stirred under Ar at ambient temperature for 13 hr. Sodium Borohydride (80.46 mg, 2.13 mmol, 74.92 μL) was added and the resulting mixture was stirred for an additional 1 hr at ambient temperature. The reaction was then quenched by pouring into aqueous ammonia (10 mL), the resulting inorganic precipitate was filtered off, and washed with dichloromethane (10 mL). The organic layer was separated, and the remaining aqueous layer was extracted once with dichloromethane. Organic phase was combined, dried over anhydrous sodium sulfate, filtered and evaporated. Resulting crude product was poured into water (20 mL) and NaHSO₄ was added to pH=3. Resulting mixture was washed with DCM (2×20 mL). Water was separated and alkalized with K₂CO₃ to pH=10. The solution was extracted with DCM (2×20 mL). Organic phase was separated, dried over anhydrous sodium sulfate, filtered and evaporated to obtain N-ethyl-1-[2-methyl-4-(trifluoromethyl)phenyl]ethanamine (132 mg, crude) as a light-yellow oil.

LCMS (ESI): [M+H]⁺m/z: calcd 231.13; found 232.2; Rt=0.693 min.

### Step 4:2,2,2-trifluoroethyl 2-[ethyl-[1-[2-methyl-4-(trifluoromethyl)phenyl]ethyl]amino]-2-oxo-acetate

To a stirred solution of N-ethyl-1-[2-methyl-4-(trifluoromethyl)phenyl]ethanamine (132 mg, 570.79 μmol) in Dichloromethane (2.91 mL) were added triethylamine (86.64 mg, 856.19 μmol, 119.34 μL) respectively at room temperature. The resulting reaction mixture was cooled to 0° C. Then 2,2,2-trifluoroethyl 2-chloro-2-oxo-acetate (130.49 mg, 684.95 μmol, 88.77 μL) was added dropwise. The reaction was stirred extra 30 minutes at 0° C., then allowed warmed to room temperature and stirred 12 hr. Upon completion, the reaction mixture was washed with water (2×20 mL). Organic phase was then dried over anhydrous sodium sulfate and filtered. The filtrate was concentrated under reduced pressure to obtain 2,2,2-trifluoroethyl 2-[ethyl-[1-[2-methyl-4-(trifluoromethyl)phenyl]ethyl]amino]-2-oxo-acetate (195 mg, crude) as a brown gum.

LCMS (ESI): [M+H]⁺m/z: calcd 385.12; found 386.0; Rt=1.596 min.

### Step 5: N′-ethyl-N′-[1-[2-methyl-4-(trifluoromethyl)phenyl]ethyl]oxamide

A solution of 2,2,2-trifluoroethyl 2-[ethyl-[1-[2-methyl-4-(trifluoromethyl)phenyl]ethyl]amino]-2-oxo-acetate (195 mg, 506.10 μmol) in Methanol/NH3 (7N, 2 mL) was stirred at 25° C. for 12 hr. The solvent was evaporated to obtain N′-ethyl-N′-[1-[2-methyl-4-(trifluoromethyl)phenyl]ethyl]oxamide (135 mg, crude) as a brown gum.

### Step 6: N-(4-amino-1-tetrahydropyran-2-yl-pyrazolo[4,3-c]pyridin-7-yl)-N′-ethyl-N′-[1-[2-methyl-4-(trifluoromethyl)phenyl]ethyl]oxamide

Copper (1.42 mg, 22.33 μmol), Copper (I) iodide (42.53 mg, 223.29 μmol, 7.57 μL), cesium carbonate (218.26 mg, 669.88 μmol) was added to a stirred solution of N′-ethyl-N′-[1-[2-methyl-4-(trifluoromethyl)phenyl]ethyl]oxamide (135 mg, 446.59 μmol), 7-bromo-1-tetrahydropyran-2-yl-pyrazolo[4,3-c]pyridin-4-amine (132.70 mg, 446.59 μmol), rac-(1R,2R)—N1,N2-dimethylcyclohexane-1,2-diamine (31.76 mg, 223.29 μmol) in 1,4-dioxane (4 mL) under Ar atmosphere and stirred at 90° C. for 48 hr in closed vial. The reaction mixture was cooled and filtered. The filtercake was washed with MeOH (10 mL) and the filtrate was concentrated in vacuo to obtain N-(4-amino-1-tetrahydropyran-2-yl-pyrazolo[4,3-c]pyridin-7-yl)-N′-ethyl-N′-[1-[2-methyl-4-(trifluoromethyl)phenyl]ethyl]oxamide (259 mg, crude) as a brown solid which was used in the next step without further purification.

LCMS (ESI): [M+H]⁺m/z: calcd 518.24; found 519.2; Rt=1.264 min.

### Step 7: N-(4-amino-1H-pyrazolo[4,3-c]pyridin-7-yl)-N′-ethyl-N′-[1-[2-methyl-4-(trifluoromethyl)phenyl]ethyl]oxamide

N-(4-amino-1-tetrahydropyran-2-yl-pyrazolo[4,3-c]pyridin-7-yl)-N′-ethyl-N′-[1-[2-methyl-4-(trifluoromethyl)phenyl]ethyl]oxamide (259 mg, 499.49 μmol) was dissolved in Dioxane/HCl (2 mL) and Methanol (2 mL). Resulting solution was stirred at rt for 12 hr. Solvents were evaporated. Resulting product was purified by HPLC (0-2-8 min 37-45-65 H₂O/R1/0.1% NH₄OH, flow 30 ml/min ((loading pump 4 ml MeOH) target mass 434; column: XBridge BEH C18 100×19 mm, 5 μM) to obtain N-(4-amino-1H-pyrazolo[4,3-c]pyridin-7-yl)-N′-ethyl-N′-[1-[2-methyl-4-(trifluoromethyl)phenyl]ethyl]oxamide (17 mg, 39.13 μmol, 7.83% yield) as a light-yellow solid.

LCMS (ESI): [M+H]⁺m/z: calcd 434.2; found 435.2; Rt=2.681 min.

### Step 8: The synthesis of (R)—N¹-(4-amino-1H-pyrazolo[4,3-c]pyridin-7-yl)-N2-ethyl-N2-(1-(2-methyl-4-(trifluoromethyl)phenyl)ethyl)oxalamide & (S)-N1-(4-amino-1H-pyrazolo[4,3-c]pyridin-7-yl)-N2-ethyl-N2-(1-(2-methyl-4-(trifluoromethyl)phenyl)ethyl)oxalamide

N-(4-amino-1H-pyrazolo[4,3-c]pyridin-7-yl)-N′-ethyl-N′-[1-[2-methyl-4-(trifluoromethyl)phenyl]ethyl]oxamide (17 mg, 39.13 μmol) was chirally separated (Column: CHIRALPAK IC (250×20 mm, 5 μm)-II; Mobile phase: Hexane (0.1% DEA): IPA: MeOH, 80:10:10; flow rate: 15 ml/min) to obtain (R)—N¹-(4-amino-1H-pyrazolo[4,3-c]pyridin-7-yl)-N2-ethyl-N2-(1-(2-methyl-4-(trifluoromethyl)phenyl)ethyl)oxalamide (4.43 mg, 10.20 μmol, Rt=18.894 min) and(S)-N1-(4-amino-1H-pyrazolo[4,3-c]pyridin-7-yl)-N2-ethyl-N2-(1-(2-methyl-4-(trifluoromethyl)phenyl)ethyl)oxalamide (5.22 mg, 12.02 μmol, Rt=27.582 min) as light-yellow solids.

### Compound 70:

¹H NMR (600 MHz, dmso) δ 0.51-0.76 (m, 3H), 1.46-1.75 (m, 3H), 2.26-2.43 (m, 3H), 2.96-3.27 (m, 2H), 5.20-5.85 (m, 1H), 6.57-6.84 (m, 2H), 7.37-7.80 (m, 4H), 8.01-8.26 (m, 1H), 9.46-10.58 (m, 1H), 12.62-13.31 (m, 1H).

LCMS (ESI): [M+H]⁺m/z: calcd 434.19; found 435.2; Rt=2.542 min.

### Compound 57:

¹H NMR (600 MHz, dmso) 8 0.53-0.75 (m, 3H), 1.48-1.71 (m, 3H), 2.33-2.40 (m, 3H), 2.99-3.25 (m, 2H), 5.27-5.91 (m, 1H), 6.51-6.96 (m, 2H), 7.44-7.82 (m, 4H), 8.03-8.25 (m, 1H), 9.49-10.59 (m, 1H), 12.57-13.31 (m, 1H).

LCMS (ESI): [M+H]⁺m/z: calcd 434.19; found 435.2; Rt=2.537 min.

### Example 71. Synthesis of N1-(4-amino-1H-pyrazolo[4,3-c]pyridin-7-yl)-N2-((5-cyanopyridin-2-yl)methyl)-N2-(pyrimidin-2-ylmethyl)oxalamide and M-(4-amino-1H-pyrazolo[4,3-c]pyridin-7-yl)-N2-((5-carbamoylpyridin-2-yl)methyl)-2-(pyrimidin-2-ylmethyl)oxalamide (Compound 113, Compound 136)

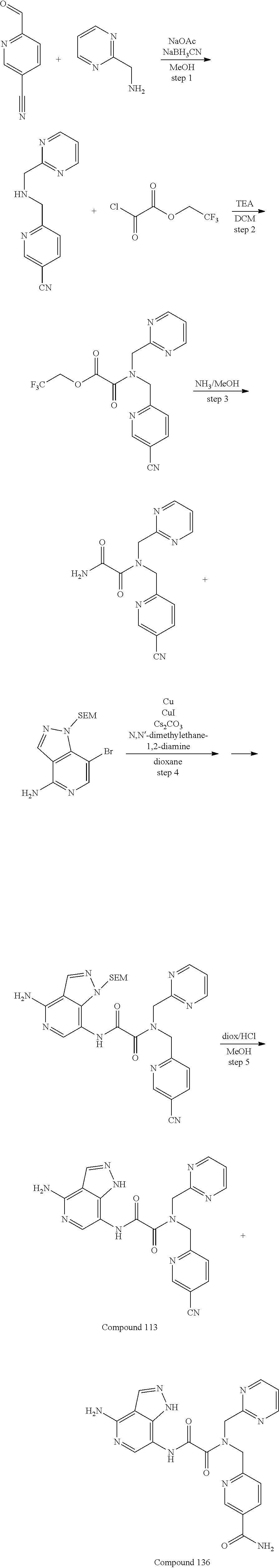

### Step 1: Synthesis of 6-(((pyrimidin-2-ylmethyl)amino)methyl) nicotinonitrile

Pyrimidin-2-ylmethanamine (5.51 g, 37.84 mmol, HCl) was dissolved in MeOH (50 mL) and sodium acetate, anhydrous (3.41 g, 41.63 mmol, 2.23 mL) was added thereto. The resulting mixture was stirred for 30 min and 6-formylpyridine-3-carbonitrile (5 g, 37.84 mmol) was added to the previous mixture followed by addition of sodium cyan borohydride (3.57 g, 56.77 mmol). The resulting mixture was stirred overnight. The reaction mixture was concentrated in vacuum and aq. NaHCO₃ was added to the residue. The resulting mixture was extracted with DCM (twice). Combined organic layers were washed with aq. NaHCO₃, dried over Na₂SO₄, filtered and concentrated in vacuum. The obtained product was used in the next step without further purification. 6-[(Pyrimidin-2-ylmethylamino)methyl]pyridine-3-carbonitrile (7.5 g, 33.30 mmol, 87.98% yield) was obtained as a red solid.

LCMS (ESI): [M]⁺m/z: calcd 225.2; found 226.2; Rt=0.343 min.

### Step 2: Synthesis of 2,2,2-trifluoroethyl 2-(((5-cyanopyridin-2-yl)methyl)(pyrimidin-2-ylmethyl)amino)-2-oxoacetate

2,2,2-Trifluoroethyl 2-chloro-2-oxo-acetate (1.26 g, 6.64 mmol) was added dropwise to a solution of 6-[(pyrimidin-2-ylmethylamino)methyl]pyridine-3-carbonitrile (1.3 g, 5.77 mmol) and TEA (671.61 mg, 6.64 mmol, 925.08 μL) in DCM (55 mL) at 0° C. After addition was complete, cooling bath was removed and resulting mixture was allowed to warm up to 20° C. and stirred for 12 hr. The reaction mixture was washed with water, dried over Na₂SO₄ and concentrated under reduced pressure. The obtained product was used in the next step without further purification. 2,2,2-Trifluoroethyl 2-[(5-cyano-2-pyridyl)methyl-(pyrimidin-2-ylmethyl)amino]-2-oxo-acetate (1.71 g, 4.51 mmol, 78.12% yield) was obtained as a red oil.

LCMS (ESI): [M]⁺m/z: calcd 379.2; found 380.2; Rt=1.064 min.

### Step 3: Synthesis of N1-((5-cyanopyridin-2-yl)methyl)-N1-(pyrimidin-2-ylmethyl)oxalamide

2,2,2-Trifluoroethyl 2-[(5-cyano-2-pyridyl)methyl-(pyrimidin-2-ylmethyl)amino]-2-oxo-acetate (1.7 g, 4.48 mmol) was dissolved in MeOH/NH3 (30 mL) as stirred overnight at 20° C. The reaction mixture was evaporated to dryness. The obtained product was used in the next step without further purification. N′-[(5-Cyano-2-pyridyl)methyl]-N′-(pyrimidin-2-ylmethyl)oxamide (1.3 g, 4.39 mmol, 97.90% yield) was obtained as a red solid.

LCMS (ESI): [M]⁺m/z: calcd 296.2; found 297.2; Rt=0.719 min.

### Step 4: Synthesis of N1-(4-amino-1-((2-(trimethylsilyl) ethoxy)methyl)-1H-pyrazolo[4,3-c]pyridin-7-yl)-N2-((5-cyanopyridin-2-yl)methyl)-N2-(pyrimidin-2-ylmethyl)oxalamide

7-Bromo-1-(2-trimethylsilylethoxymethyl)pyrazolo[4,3-c]pyridin-4-amine (380 mg, 1.11 mmol), N′-[(5-cyano-2-pyridyl)methyl]-N′-(pyrimidin-2-ylmethyl)oxamide (327.96 mg, 1.11 mmol), Cu(7.46 mg, 117.33 μmol), CuI (63.24 mg, 332.08 μmol, 11.25 μL), cesium carbonate (540.99 mg, 1.66 mmol, 236.24 μL) and (1R,2R)—N1,N2-dimethylcyclohexane-1,2-diamine (47.23 mg, 332.08 μmol) were mixed in dioxane (5 mL), purged with Ar for 5 minutes and then heated in the sealed tube at 100° C. for 18 hr. Final mixture was filtered and dioxane was evaporated in vacuum. The residue was purified by RP-HPLC (column: XBridge BEH C18 5 μm 130A; 25-25-40% 0-1-6 min H₂O/meCN/0.1% NH₄OH, flow: 30 ml/min) to afford N-[4-amino-1-(2-trimethylsilylethoxymethyl)pyrazolo[4,3-c]pyridin-7-yl]-N′-[(5-cyano-2-pyridyl)methyl]-N′-(pyrimidin-2-ylmethyl)oxamide (62 mg, 110.98 μmol, 10.03% yield) as light-brown solid (mixture of cis/trans), which was used directly in the next step.

LCMS (ESI): [M]⁺m/z: calcd 558.2; found 559.2; Rt=1.178 min.

### Step 5: Synthesis of N1-(4-amino-1H-pyrazolo[4,3-c]pyridin-7-yl)-N2-((5-cyanopyridin-2-yl)methyl)-N2-(pyrimidin-2-ylmethyl)oxalamide and N1-(4-amino-1H-pyrazolo[4,3-c]pyridin-7-yl)-N2-((5-carbamoylpyridin-2-yl)methyl)-N2-(pyrimidin-2-ylmethyl)oxalamide (Compound 113 and Compound 136)

To a solution of N-[4-amino-1-(2-trimethylsilylethoxymethyl)pyrazolo[4,3-c]pyridin-7-yl]-N′-[(5-cyano-2-pyridyl)methyl]-N′-(pyrimidin-2-ylmethyl)oxamide (62 mg, 110.98 μmol) in MeOH (4 mL) was added hydrogen chloride solution 4.0M in dioxane (606.94 mg, 1.66 mmol, 758.68 μL, 10% purity) at 21° C. The resulting mixture was left to stir for 18 hr. The resulting mixture was evaporated to dryness. The residue was purified by RP-HPLC (column: Chromatorex 18 SMB100-5T 100×19 mm 5 μm; 0-0-25% 0-2-5 min H₂O/MeCN/0.1% FA, flow: 30 ml/min) to give Compound 113 N-(4-amino-1H-pyrazolo[4,3-c]pyridin-7-yl)-N′-[(5-cyano-2-pyridyl)methyl]-N′-(pyrimidin-2-ylmethyl)oxamide (11.5 mg, 22.10 μmol, 19.91% yield, 2HCOOH) and Compound 136 N-(4-amino-1H-pyrazolo[4,3-c]pyridin-7-yl)-N′-[(5-carbamoyl-2-pyridyl)methyl]-N′-(pyrimidin-2-ylmethyl)oxamide (17.8 mg, 33.06 μmol, 2HCOOH) as by-product.

### Compound 113:

¹H NMR (600 MHz, DMSO-d₆) δ (ppm) 4.80-4.93 (m, 2H), 5.19-5.29 (m, 2H), 6.59-6.70 (m, 2H), 7.33-7.43 (m, 1H), 7.57-7.62 (m, 1H), 7.62-7.72 (m, 1H), 8.14-8.15 (m, 1H), 8.24-8.35 (m, 1H), 8.71-8.81 (m, 2H), 8.92-8.99 (m, 1H), 10.40-10.54 (m, 1H), 12.65-12.97 (m, 1H).

LCMS (ESI): [M]⁺m/z: calcd 428.2; found 429.2; Rt=1.878 min.

### Compound 136:

¹H NMR (600 MHz, DMSO-d₆) δ (ppm) 4.79-4.87 (m, 2H), 5.17-5.24 (m, 2H), 7.33-7.44 (m, 2H), 7.49-7.58 (m, 2H), 7.67-7.73 (m, 1H), 8.08-8.15 (m, 2H), 8.16-8.21 (m, 1H), 8.26-8.34 (m, 1H), 8.73-8.77 (m, 1H), 8.77-8.81 (m, 1H), 8.92-8.95 (m, 1H), 10.53-10.65 (m, 1H), 13.17 (s, 1H).

LCMS (ESI): [M]⁺m/z: calcd 446.2; found 447.2; Rt=1.445 min.

### Example 72. Synthesis of The synthesis of N1-(4-amino-1H-pyrazolo[4,3-c]pyridin-7-yl)-N2-((5-cyanopyridin-2-yl)methyl)-N2-(1-(pyrimidin-2-yl)ethyl)oxalamide (Compound 135)

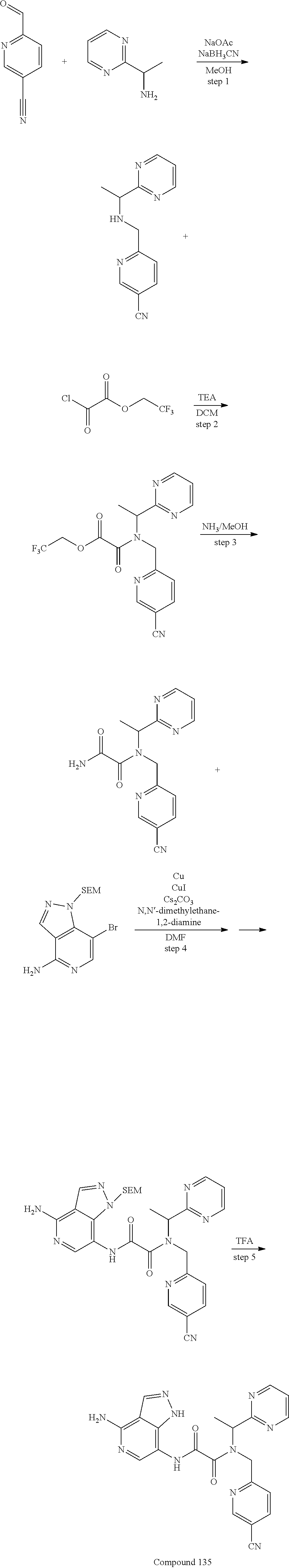

### Step 1: Synthesis of 6-(((1-(pyrimidin-2-yl)ethyl)amino)methyl) nicotinonitrile

1-Pyrimidin-2-ylethanamine (7.42 g, 37.84 mmol, 2HCl) was dissolved in MeOH (50 mL) and sodium acetate, anhydrous (6.52 g, 79.47 mmol, 4.27 mL) was added thereto. The resulting mixture was stirred for 30 min and 6-formylpyridine-3-carbonitrile (5 g, 37.84 mmol) was added to the previous mixture followed by addition of sodium cyan borohydride (3.57 g, 56.77 mmol). The resulting mixture was stirred overnight. The reaction mixture was concentrated in vacuum and aq. NaHCO₃ was added to the residue. The resulting mixture was extracted with DCM (twice). Combined organic layers were washed with aq. NaHCO₃, dried over Na₂SO₄, filtered and concentrated in vacuum. The obtained product was used in the next step without further purification. 6-[(1-pyrimidin-2-ylethylamino)methyl]pyridine-3-carbonitrile (8 g, 33.43 mmol, 88.35% yield) was obtained as a red oil.

LCMS (ESI): [M]⁺m/z: calcd 239.2; found 240.2; Rt=0.435 min.

### Step 2: Synthesis of 2,2,2-trifluoroethyl 2-(((5-cyanopyridin-2-yl)methyl)(1-(pyrimidin-2-yl)ethyl)amino)-2-oxoacetate

2,2,2-Trifluoroethyl 2-chloro-2-oxo-acetate (796.17 mg, 4.18 mmol) was added dropwise to a solution of 6-[(1-pyrimidin-2-ylethylamino)methyl]pyridine-3-carbonitrile (1 g, 4.18 mmol) and TEA (486.34 mg, 4.81 mmol, 669.89 μL) in DCM (40 mL) at 0° C. After addition was complete, cooling bath was removed and resulting mixture was allowed to warm up to 20° C. and stirred for 12 hr. The reaction mixture was washed with water, dried over Na₂SO₄ and concentrated under reduced pressure. The obtained product was used in the next step without further purification. 2,2,2-Trifluoroethyl 2-[(5-cyano-2-pyridyl)methyl-(1-pyrimidin-2-ylethyl)amino]-2-oxo-acetate (1.37 g, 3.48 mmol, 83.34% yield) was obtained as a red oil.

LCMS (ESI): [M]⁺m/z: calcd 393.2; found 394.2; Rt=1.194 min.

### Step 3: Synthesis of N1-((5-cyanopyridin-2-yl)methyl)-N1-(1-(pyrimidin-2-yl)ethyl)oxalamide

2,2,2-Trifluoroethyl 2-[(5-cyano-2-pyridyl)methyl-(1-pyrimidin-2-ylethyl)amino]-2-oxo-acetate (1.37 g, 3.48 mmol) was dissolved in NH3/MeOH (30 mL) as stirred overnight at 20° C. The reaction mixture was evaporated to dryness. The crude product was purified by FCC (Methanol in MTBE from 2% to 8%). N′-[(5-Cyano-2-pyridyl)methyl]-N′-(1-pyrimidin-2-ylethyl)oxamide (0.4 g, 1.29 mmol, 37.01% yield) was obtained as a light-yellow solid.

LCMS (ESI): [M]⁺m/z: calcd 310.2; found 311.2; Rt=0.669 min.

### Step 4: Synthesis of N1-(4-amino-1-((2-(trimethylsilyl) ethoxy)methyl)-1H-pyrazolo[4,3-c]pyridin-7-yl)-N2-((5-cyanopyridin-2-yl)methyl)-N2-(1-(pyrimidin-2-yl)ethyl)oxalamide

N′-[(5-Cyano-2-pyridyl)methyl]-N′-(1-pyrimidin-2-ylethyl)oxamide (0.4 g, 1.29 mmol), 7-bromo-1-(2-trimethylsilylethoxymethyl)pyrazolo[4,3-c]pyridin-4-amine (442.52 mg, 1.29 mmol), copper (I) iodide (245.50 mg, 1.29 mmol, 43.68 μL), cesium carbonate (839.98 mg, 2.58 mmol) and (1R,2R)—N1,N2-dimethylcyclohexane-1,2-diamine (275.03 mg, 1.93 mmol) were mixed in DMF (10 mL) under argon, and then stirred overnight at 100° C. for 12 hr in vial. The reaction mixture was submitted to HPLC (2-10 min 30-60% MeCN+FA 30 ml/min; loading pump 4 ml/min MeCN+FA column SunFire 19*100 mm). N-[4-Amino-1-(2-trimethylsilylethoxymethyl)pyrazolo[4,3-c]pyridin-7-yl]-N′-[(5-cyano-2-pyridyl)methyl]-N′-(1-pyrimidin-2-ylethyl)oxamide (0.005 g, 8.73 μmol, 6.77e-1% yield) was obtained as a brown solid.

LCMS (ESI): [M]⁺m/z: calcd 572.2; found 573.2; Rt=1.248 min.

### Step 5: Synthesis of N1-(4-amino-1H-pyrazolo[4,3-c]pyridin-7-yl)-N2-((5-cyanopyridin-2-yl)methyl)-N2-(1-(pyrimidin-2-yl)ethyl)oxalamide (Compound 135)

N-[4-Amino-1-(2-trimethylsilylethoxymethyl)pyrazolo[4,3-c]pyridin-7-yl]-N′-[(5-cyano-2-pyridyl)methyl]-N′-(1-pyrimidin-2-ylethyl)oxamide (0.005 g, 8.73 μmol) was dissolved in TFA (29.86 mg, 261.92 μmol, 20.18 μL) and stirred at 20° C. for 3 hr. The reaction mixture was submitted to HPLC (2-10 min 30-60% MeOH+NH3 30/min; loading pump 4 ml/min MeOH column SunFire 19*100 mm). N-(4-Amino-1H-pyrazolo[4,3-c]pyridin-7-yl)-N′-[(5-cyano-2-pyridyl)methyl]-N′-(1-pyrimidin-2-ylethyl)oxamide (0.0017 g, 3.84 μmol, 44.01% yield) was obtained as a light-yellow solid.

### Compound 135:

¹H NMR (600 MHz, DMSO-d₆) δ (ppm) 1.24-1.67 (m, 3H), 4.50-4.93 (m, 1H), 5.00-5.41 (m, 1H), 5.63-5.98 (m, 1H), 6.60-7.02 (m, 2H), 7.32-7.43 (m, 1H), 7.45-7.74 (m, 2H), 8.11-8.15 (m, 1H), 8.17-8.26 (m, 1H), 8.67-8.80 (m, 2H), 8.83-8.97 (m, 1H), 9.77-10.64 (m, 1H), 12.62-12.92 (m, 1H).

LCMS (ESI): [M]⁺m/z: calcd 442.2; found 443.2; Rt=1.629 min.

### Example 73. Synthesis of N-(4-amino-1H-pyrazolo[4,3-c]pyridin-7-yl)-N′-(p-tolylmethyl)-N′-(2-pyridylmethyl)oxamide (Compound 190)

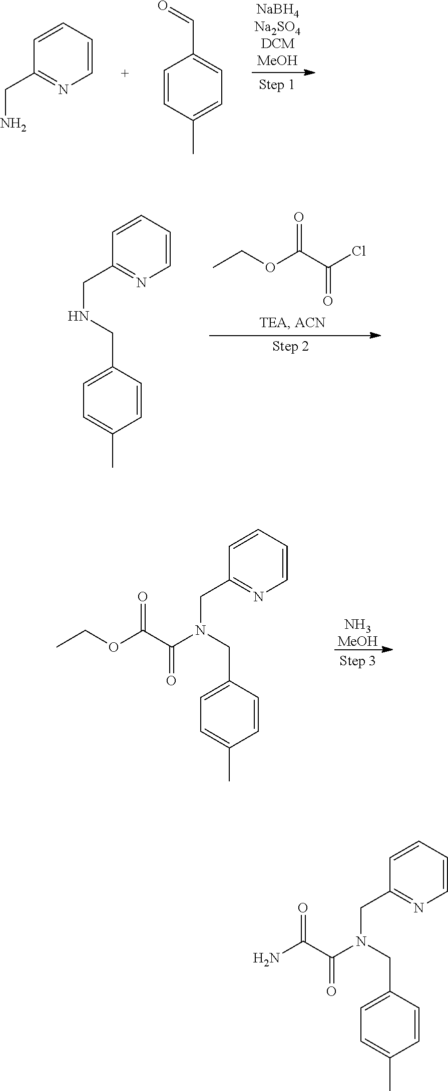

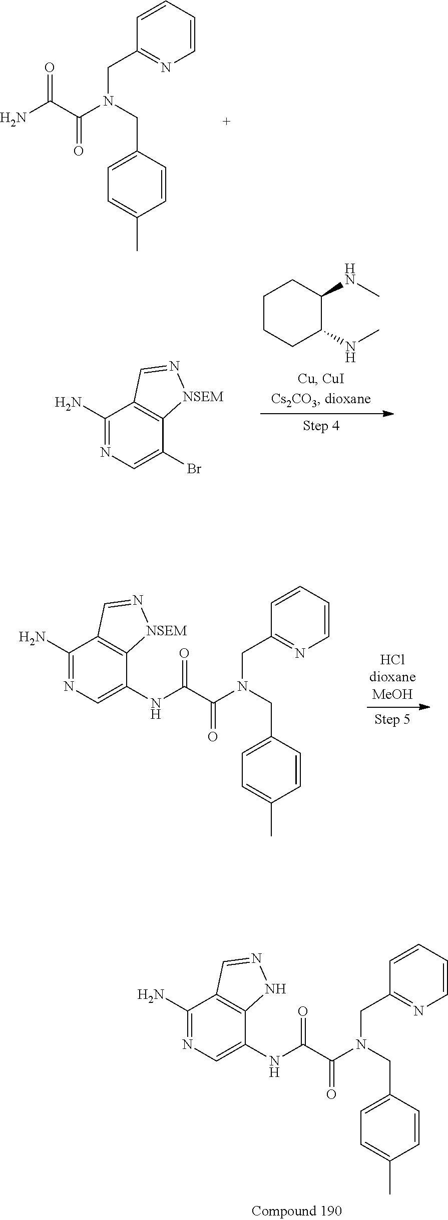

### Step 1:1-(p-tolyl)-N-(2-pyridylmethyl)methanamine

2-pyridylmethanamine (1 g, 9.25 mmol, 953.29 μL) and 4-methylbenzaldehyde (1.11 g, 9.25 mmol) were mixed in DCM (20 mL) at 20° C. The resulting mixture was stirred at 20° C. for 12 hr, then filtered and concentrated in vacuo. The residue was dissolved in methanol (20 mL), cooled to 5° C. and Sodium Borohydride (384.80 mg, 10.17 mmol, 358.29 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford 1-(p-tolyl)-N-(2-pyridylmethyl)methanamine (1.5 g, crude) as a yellow oil.

LCMS (ESI): [M+H]⁺m/z: calcd 213.14; found 213.2; Rt=0.587 min.

### Step 2: ethyl 2-oxo-2-[p-tolylmethyl(2-pyridylmethyl)amino]acetate

1-(p-tolyl)-N-(2-pyridylmethyl)methanamine (1.5 g, 5.30 mmol) and TEA (1.07 g, 10.60 mmol, 1.48 mL) were dissolved in acetonitrile (20 mL), cooled with ice-water bath and then ethyl 2-chloro-2-oxoacetate (1.09 g, 7.95 mmol, 888.14 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-oxo-2-[p-tolylmethyl(2-pyridylmethyl)amino]acetate (1.9 g, crude) as a red oil.

LCMS (ESI): [M+H]⁺m/z: calcd 313.16; found 313.0; Rt=1.182 min.

### Step 3: N′-(p-tolylmethyl)-N′-(2-pyridylmethyl)oxamide

Ethyl 2-oxo-2-[p-tolylmethyl(2-pyridylmethyl)amino]acetate (1.9 g, 5.47 mmol) was dissolved in MeOH (20 mL) saturated with NH3 (5.47 mmol) and stirred overnight. The RM was concentrated, redissolved in DCM, washed with water, dried over anhydrous sodium sulfate and concentrated in vacuo to give N′-(p-tolylmethyl)-N′-(2-pyridylmethyl)oxamide (1.65 g, crude) as a brown solid.

LCMS (ESI): [M+H]⁺m/z: calcd 284.14; found 284.2; Rt=0.910 min.

### Step 4: N-[4-amino-1-(2-trimethylsilylethoxymethyl)pyrazolo[4,3-c]pyridin-7-yl]-N′-(p-tolylmethyl)-N′-(2-pyridylmethyl)oxamide

Copper (2.02 mg, 31.77 μmol), Copper (I) iodide (60.50 mg, 317.66 μmol, 10.76 μL), cesium carbonate (414.00 mg, 1.27 mmol) were added to a stirred solution of N′-(p-tolylmethyl)-N′-(2-pyridylmethyl)oxamide (200 mg, 635.31 μmol), 7-bromo-1-(2-trimethylsilylethoxymethyl)pyrazolo[4,3-c]pyridin-4-amine (218.10 mg, 635.31 μmol) and rac-(1R,2R)—N1,N2-dimethylcyclohexane-1,2-diamine (176.22 mg, 1.24 mmol) in 1,4-dioxane (4.99 mL) under Ar atmosphere and stirred at 100° C. for 12 hr in closed vial. 105 mg of starting bromide was added additionally under argon and heated with stirring next 22 hr. Reaction mixture was filtered, solid washed with dioxane (2×3 mL), filtrate concentrated. The residue was treated with DCM (30 mL), washed with water twice, dried over anhydrous sodium sulfate and evaporated in vacuo to give N-[4-amino-1-(2-trimethylsilylethoxymethyl)pyrazolo[4,3-c]pyridin-7-yl]-N′-(p-tolylmethyl)-N′-(2-pyridylmethyl)oxamide (450 mg, crude) as a brown oil.

LCMS (ESI): [M+H]⁺m/z: calcd 546.27; found 546.2; Rt=1.072 min.

### Step 5: The synthesis of N-(4-amino-1H-pyrazolo[4,3-c]pyridin-7-yl)-N′-(p-tolylmethyl)-N′-(2-pyridylmethyl)oxamide

N-[4-amino-1-(2-trimethylsilylethoxymethyl)pyrazolo[4,3-c]pyridin-7-yl]-N′-(p-tolylmethyl)-N′-(2-pyridylmethyl)oxamide (450 mg, 338.09 μmol) was dissolved in Dioxane (4 mL) saturated with HCl (10% by weight). MeOH (4 mL) was added for better solubility. The RM was stirred for 12 hr at 20° C. then concentrated in vacuo. The residue was dissolved in 3 ml of MeOH and subjected to HPLC (Device (Mobile Phase, Column): 2-10 min 0-15% ACN+FA) to afford N-(4-amino-1H-pyrazolo[4,3-c]pyridin-7-yl)-N′-(p-tolylmethyl)-N′-(2-pyridylmethyl)oxamide (50.5 mg, 121.56 μmol, 35.95% yield) as a light-brown solid.

¹H NMR (600 MHz, dmso) 8 2.15-2.33 (m, 3H), 4.02-4.89 (m, 4H), 5.78-6.98 (m, 2H), 7.13-7.41 (m, 5H), 7.54-7.70 (m, 1H), 7.73-7.84 (m, 1H), 8.00-8.35 (m, 2H), 8.46-8.56 (m, 1H), 9.67-10.68 (m, 1H), 12.49-13.64 (m, 1H).

LCMS (ESI): [M+H]⁺m/z: calcd 416.2; found 416.2; Rt=1.029 min.

### Example 74. Synthesis of N-(4-amino-1H-pyrazolo[4,3-c]pyridin-7-yl)-N′-benzyl-N′-[(2-methylpyrazol-3-yl)methyl]oxamide (Compound 125)

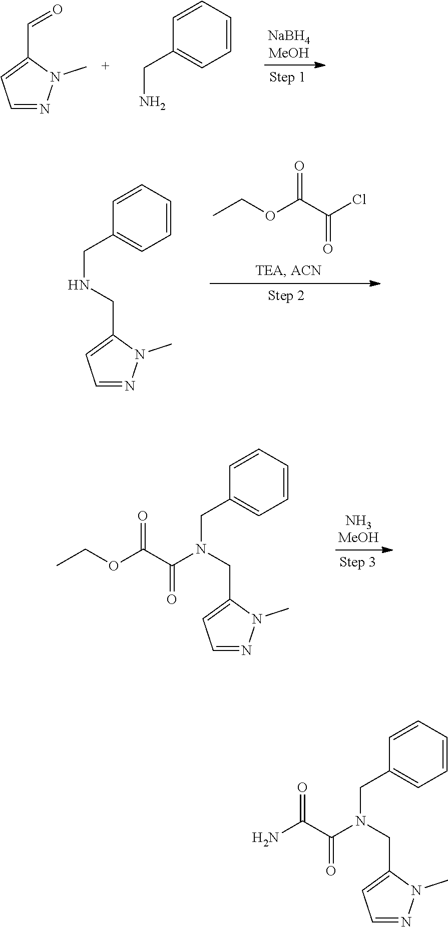

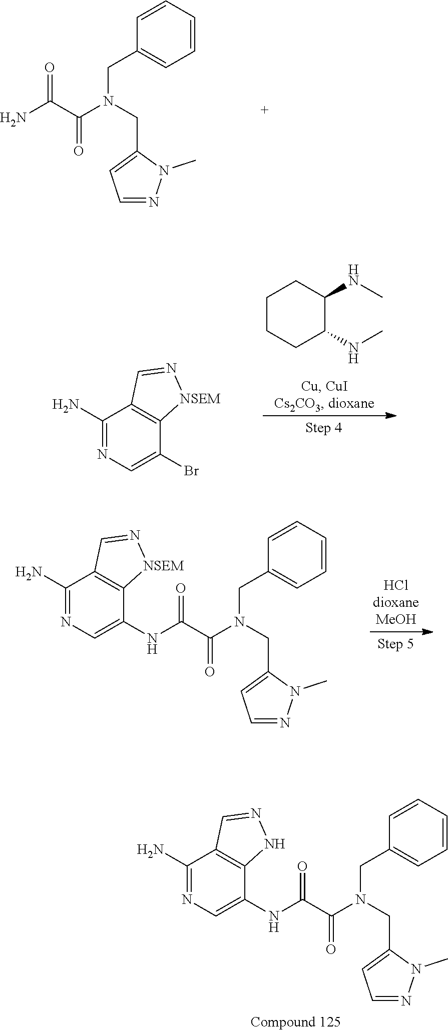

### Step 1: N-[(2-methylpyrazol-3-yl)methyl]-1-phenyl-methanamine

Phenylmethanamine (5 g, 46.66 mmol) phenylmethanamine (5 g, 46.66 mmol) was added to a stirred solution of 2-methylpyrazole-3-carbaldehyde (5.14 g, 46.66 mmol) in Methanol (50 mL) stirred at 20° C. for 10 hr. Sodium Borohydride (1.77 g, 46.66 mmol, 1.64 mL) was added, the reaction mixture was stirred for 2 hr additional. The reaction mixture was evaporated in vacuo. The residue was dissolved in DCM (50 mL) and washed with water (2×15 mL). The organic layer was dried over anhydrous sodium sulfate and filtered. DCM was evaporated under reduce pressure to give N-[(2-methylpyrazol-3-yl)methyl]-1-phenyl-methanamine (4.1 g, 20.37 mmol, 43.66% yield) as a brown oil.

LCMS (ESI): [M+H]⁺m/z: calcd 202.14; found 202.0; Rt=0.384 min.

### Step 2: ethyl 2-[benzyl-[(2-methylpyrazol-3-yl)methyl]amino]-2-oxo-acetate

N-[(2-methylpyrazol-3-yl)methyl]-1-phenyl-methanamine (2 g, 9.94 mmol) and TEA (1.01 g, 9.94 mmol, 1.39 mL) dissolved in DCM (50 mL), cooled with ice-water bath and then ethyl 2-chloro-2-oxo-acetate (1.36 g, 9.94 mmol, 1.11 mL) in 20 mL of DCM was added dropwise in 10 min. The solution was stirred overnight. The reaction mixture was washed with an aqueous solution of NaHCO₃, dried over anhydrous sodium sulfate and evaporated to give ethyl 2-[benzyl-[(2-methylpyrazol-3-yl)methyl]amino]-2-oxo-acetate (2.1 g, 6.97 mmol, 70.13% yield) as a yellow oil.

LCMS (ESI): [M+H]⁺m/z: calcd 302.15; found 302.2; Rt=1.078 min.

### Step 3: N′-benzyl-N′-[(2-methylpyrazol-3-yl)methyl]oxamide

Ethyl 2-[benzyl-[(2-methylpyrazol-3-yl)methyl]amino]-2-oxo-acetate (2.1 g, 6.97 mmol) was dissolved in NH3/methanol (30 mL) solution (10% by weight) and stirred overnight, then concentrated in vacuo to give N′-benzyl-N′-[(2-methylpyrazol-3-yl)methyl]oxamide (1.8 g, 6.61 mmol, 94.85% yield) as a yellow oil.

LCMS (ESI): [M+H]⁺m/z: calcd 273.14; found 273.2; Rt=0.814 min.

### Step 4: N-[4-amino-1-(2-trimethylsilylethoxymethyl)pyrazolo[4,3-c]pyridin-7-yl]-N′-benzyl-N′-[(2-methylpyrazol-3-yl)methyl]oxamide

7-bromo-1-(2-trimethylsilylethoxymethyl)pyrazolo[4,3-c]pyridin-4-amine (252.14 mg, 734.48 μmol), N′-benzyl-N′-[(2-methylpyrazol-3-yl)methyl]oxamide (0.2 g, 734.48 μmol), Copper (I) iodide (41.96 mg, 220.34 μmol, 7.47 μL), Cesium carbonate (478.62 mg, 1.47 mmol) and (1R,2R)—N1,N2-dimethylcyclohexane-1,2-diamine (47.01 mg, 330.52 μmol) were mixed in Dioxane under argon, and then stirred overnight at 100° C. for 12 hr in vial. The reaction mixture was filtered, then evaporated. The residue was dissolved in 15 mL of DCM washed with an aqueous ammonium solution, dried over anhydrous sodium sulfate and evaporated to give N-[4-amino-1-(2-trimethylsilylethoxymethyl)pyrazolo[4,3-c]pyridin-7-yl]-N′-benzyl-N′-[(2-methylpyrazol-3-yl)methyl]oxamide (0.35 g, 654.59 μmol, 89.12% yield).

LCMS (ESI): [M+H]⁺m/z: calcd 535.26; found 535.2; Rt=0.916 min.

### Step 5: The synthesis of N-(4-amino-1H-pyrazolo[4,3-c]pyridin-7-yl)-N′-benzyl-N′-[(2-methylpyrazol-3-yl)methyl]oxamide

N-[4-amino-1-(2-trimethylsilylethoxymethyl)pyrazolo[4,3-c]pyridin-7-yl]-N′-benzyl-N′-[(2-methylpyrazol-3-yl)methyl]oxamide (0.35 g, 654.59 μmol) was dissolved in MeOH (1 mL) and dioxane/HCl (13.09 mmol, 1 mL) was added thereto. Then it was stirred at rt for 2 hr. The reaction mixture was evaporated. Crude product was purified by HPLC (Device (Mobile Phase, Column): 2-10 min 30-60% methanol+NH3 30 mL) to afford N-(4-amino-1H-pyrazolo[4,3-c]pyridin-7-yl)-N′-benzyl-N′-[(2-methylpyrazol-3-yl)methyl]oxamide (0.039 g, 96.43 μmol, 14.73% yield) as a yellow solid.

¹H NMR (600 MHz, cd₃ od) δ 3.65-3.84 (m, 3H), 4.67-4.81 (m, 2H), 4.85-4.99 (m, 2H), 6.21-6.30 (m, 1H), 7.23-7.40 (m, 6H), 7.60-7.74 (m, 1H), 8.23-8.29 (m, 1H).

LCMS (ESI): [M+H]⁺m/z: calcd 405.19; found 405.2; Rt=1.801 min.

### Example 75. Synthesis of N-(4-amino-1H-pyrazolo[4,3-c]pyridin-7-yl)-N′-(2-methylbutyl)-N′-(2-pyridylmethyl)oxamide (Compound 152)

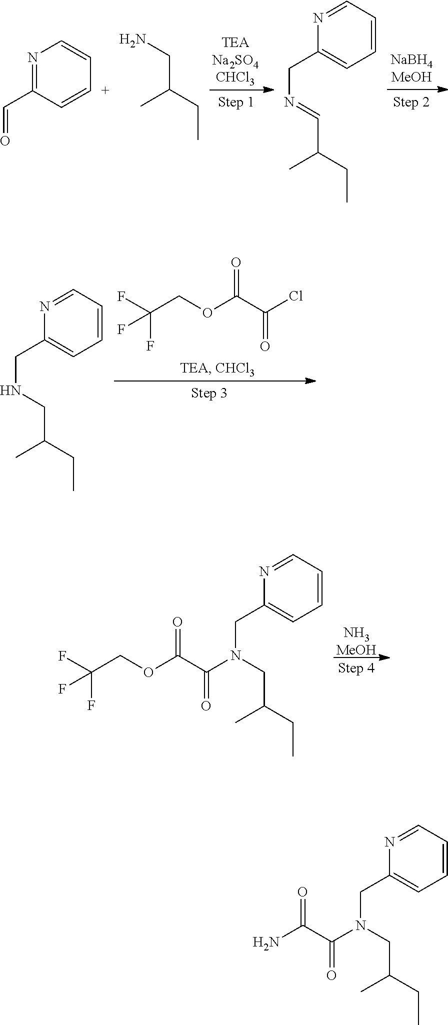

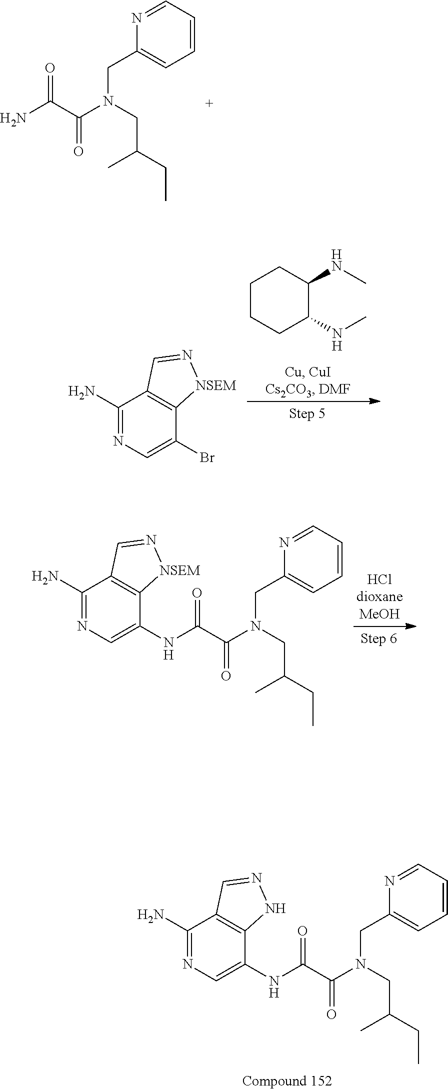

### Step 1: (E)-N-(2-methylbutyl)-1-(2-pyridyl) methanimine

To a stirred solution of pyridine-2-carbaldehyde (1 g, 9.34 mmol, 889.68 μL) and 2-methylbutan-1-amine (1.5 g, 12.13 mmol, 1.97 mL, HCl) in CHCl₃ (20 mL) were added Triethylamine (1.45 g, 14.35 mmol, 2 mL) and Sodium sulfate, anhydrous (7 g, 49.28 mmol, 2.61 mL). The resulting reaction mixture was stirred at 25° C. for 16 hr. Upon completion, the reaction mixture was filtered, the filtrate was quenched with water (20 mL). The aqueous phase was extracted with CHCl₃ (20 mL). The combined organic phase was dried over anhydrous sodium sulfate and concentrated under reduced pressure to afford (E)-N-(2-methylbutyl)-1-(2-pyridyl) methanimine (1.6 g, 9.08 mmol, 97.23% yield) as a colorless oil.

### Step 2:2-methyl-N-(2-pyridylmethyl) butan-1-amine

To a stirred solution of (E)-N-(2-methylbutyl)-1-(2-pyridyl) methanimine (1.6 g, 9.08 mmol) in MeOH (50 mL) was added Sodium Borohydride (0.5 g, 13.22 mmol, 465.55 μL). The resulting reaction mixture was stirred at 25° C. for 16 hr. Upon completion, the reaction mixture was evaporated, quenched with water (20 mL). The aqueous phase was extracted with CHCI3 (2×20 mL). The combined organic phase was dried over anhydrous sodium sulfate and concentrated under reduced pressure to afford 2-methyl-N-(2-pyridylmethyl) butan-1-amine (1.5 g, 8.41 mmol, 92.69% yield) as a yellow oil.

### Step 3:2,2,2-trifluoroethyl 2-[2-methylbutyl(2-pyridylmethyl)amino]-2-oxo-acetate

To a stirred solution of 2-methyl-N-(2-pyridylmethyl) butan-1-amine (0.5 g, 2.80 mmol) and Triethylamine (435.60 mg, 4.30 mmol, 0.6 mL) in CHCl₃ (10 mL) was added 2,2,2-trifluoroethyl 2-chloro-2-oxo-acetate (0.7 g, 3.67 mmol). The resulting reaction mixture was stirred at 25° C. for 16 hr. Upon completion, the reaction mixture was evaporated, quenched with water (20 mL). The aqueous phase was extracted with CHCl₃ (2×20 mL). The combined organic phase was dried over anhydrous sodium support and concentrated under reduced pressure to afford 2,2,2-trifluoroethyl 2-[2-methylbutyl(2-pyridylmethyl)amino]-2-oxo-acetate (0.85 g, 2.56 mmol, 91.20% yield) as a yellow oil.

LCMS (ESI): [M+H]⁺m/z: calcd 333.14; found 333.2; Rt=1.432 min.

### Step 4: N′-(2-methylbutyl)-N′-(2-pyridylmethyl)oxamide

To a stirred solution of 2,2,2-trifluoroethyl 2-[2-methylbutyl(2-pyridylmethyl)amino]-2-oxo-acetate (0.85 g, 2.56 mmol) in MeOH (10 mL) was added NH3/MeOH (5 mL). The resulting reaction mixture was stirred at 25° C. for 6 hr. Upon completion, the reaction mixture was concentrated under reduced pressure to afford N′-(2-methylbutyl)-N′-(2-pyridylmethyl)oxamide (0.6 g, 2.41 mmol, 94.09% yield) as a white solid.

LCMS (ESI): [M+H]⁺m/z: calcd 250.16; found 250.2; Rt=0.838 min.

### Step 5: N-[4-amino-1-(2-trimethylsilylethoxymethyl)pyrazolo[4,3-c]pyridin-7-yl]-N′-(2-methylbutyl)-N′-(2-pyridylmethyl)oxamide

N′-(2-methylbutyl)-N′-(2-pyridylmethyl)oxamide (0.15 g,601.67 μmol), 7-bromo-1-(2-trimethylsilylethoxymethyl)pyrazolo[4,3-c]pyridin-4-amine (0.2 g, 582.59 μmol), Cu(30 mg, 472.07 μmol), Cesium carbonate (0.4 g, 1.23 mmol) and Copper (I) iodide (110 mg, 577.58 μmol, 19.57 μL) were mixed together in DMF (5 mL). The resulting suspension was degassed with argon at 25° C. for 0.1 hr. (1R,2R)—N1,N2-dimethylcyclohexane-1,2-diamine (180.00 mg, 1.27 mmol, 0.2 mL) was added thereto and the resulting mixture was stirred for 16 hr at 100° C. After completion the reaction mixture was filtered and the filtrate was concentrated in vacuum. The crude product was purified by silica gel column chromatography using ethylacetate/MeOH (10:1, v: v) as eluent (Rt=0.5) to afford N-[4-amino-1-(2-trimethylsilylethoxymethyl)pyrazolo[4,3-c]pyridin-7-yl]-N′-(2-methylbutyl)-N′-(2-pyridylmethyl)oxamide (0.1 g, 195.43 μmol, 32.48% yield) as a brown oil.

LCMS (ESI): [M+H]⁺m/z: calcd 512.28; found 512.2; Rt=1.259 min.

### Step 6: The synthesis of N-(4-amino-1H-pyrazolo[4,3-c]pyridin-7-yl)-N′-(2-methylbutyl)-N′ (2-pyridylmethyl)oxamide

To a stirred solution of N-[4-amino-1-(2-trimethylsilylethoxymethyl)pyrazolo[4,3-c]pyridin-7-yl]-N′-(2-methylbutyl)-N′-(2-pyridylmethyl)oxamide (0.1 g, 195.43 μmol) in MeOH (4 mL) was added Dioxane/HCl (2 mL). The resulting reaction mixture was stirred at 25° C. for 3 hr. Upon completion, the reaction mixture was concentrated under reduced pressure. The obtained crude product was purified by reverse phase HPLC chromatography (Device (Mobile Phase, Column): SYSTEM May 5, 2030% 0-1-5 min H₂O/ACN/0.1% FA, flow: 30 mL/min (loading pump 4 mL/min acetonitrile) target mass 381.44 column: Chromatorex 18 SMB100-5T 100×19 mm 5 μm) to afford product N-(4-amino-1H-pyrazolo[4,3-c]pyridin-7-yl)-N′-(2-methylbutyl)-N′-(2-pyridylmethyl)oxamide (36 mg, 84.22 μmol, 43.09% yield, HCOOH) as a white solid.

¹H NMR (600 MHz, dmso) 8 0.71-0.85 (m, 6H), 0.91-1.14 (m, 1H), 1.22-1.41 (m, 1H), 1.62-1.82 (m, 1H), 3.14-3.26 (m, 1H), 3.41-3.54 (m, 1H), 4.42-5.04 (m, 2H), 6.58-6.93 (m, 2H), 7.27-7.52 (m, 2H), 7.58-7.81 (m, 2H), 8.03-8.63 (m, 3H), 9.54-10.54 (m, 1H), 12.53-13.44 (m, 1H).

LCMS (ESI): [M+H]⁺m/z: calcd 382.22; found 382.2; Rt=2.198 min.

### Example 76. The synthesis of (R)—N¹-(4-amino-1H-pyrazolo[4,3-c]pyridin-7-yl)-N2-(2-methylbutyl)-N2-(pyridin-2-ylmethyl)oxalamide (Compound 115)

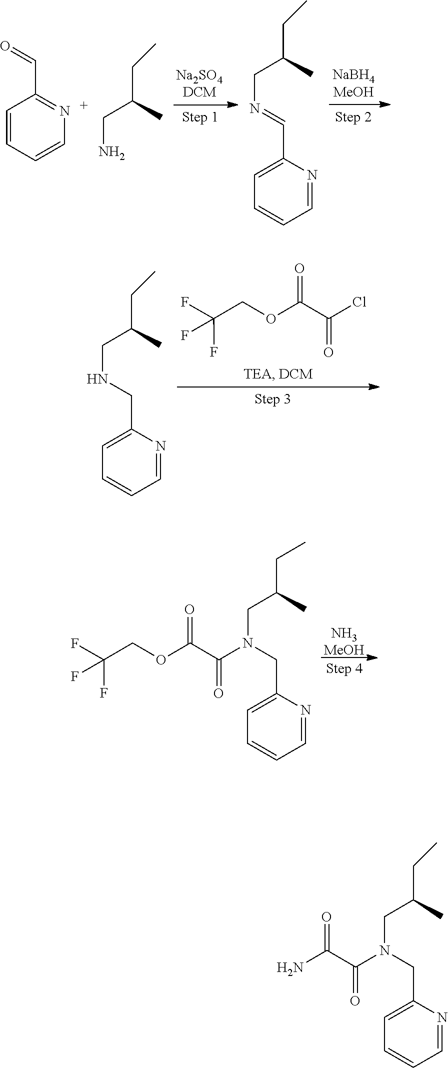

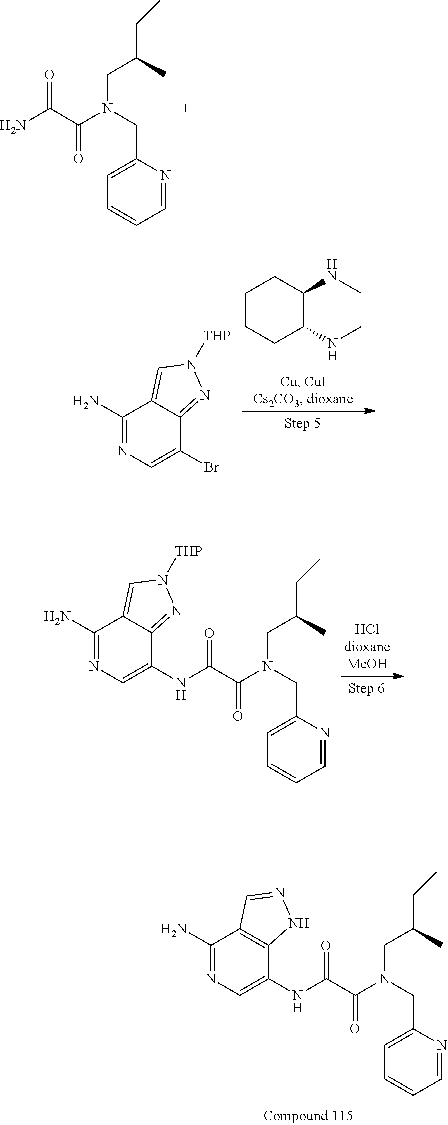

### Step 1: (R,E)-2-methyl-N-(pyridin-2-ylmethylene) butan-1-amine

pyridine-2-carbaldehyde (368.65 mg, 3.44 mmol, 327.98 μL) and (R)-2-methylbutan-1-amine (0.3 g, 3.44 mmol) were dissolved in DCM (3.57 mL), then Sodium sulfate, anhydrous (4.89 g, 34.42 mmol, 1.82 mL) was added and the reaction mixture was stirred overnight at 25° C. The reaction mixture was filtered, solid was washed with DCM and filtrate was concentrated on vacuo to (R,E)-2-methyl-N-(pyridin-2-ylmethylene) butan-1-amine (600 mg, crude) as a yellow oil which was used in the next step without further purification.

### Step 2: (R)-2-methyl-N-(pyridin-2-ylmethyl) butan-1-amine

(R,E)-2-methyl-N-(pyridin-2-ylmethylene) butan-1-amine (0.6 g, 3.40 mmol) was dissolved in Methanol (12 mL) and Sodium borohydride (128.79 mg, 3.40 mmol, 119.91 μL) was added portionwise. After the addition was completed, the reaction mixture was stirred for 12 hr. Water (5 mL) was added to the reaction mixture and the resulting mixture was concentrated in vacuo. Water (20 mL) was added to the residue and the resulting mixture was extracted with DCM (2×25 mL). Combined organic layers were dried over anhydrous sodium sulfate, filtered, and concentrated in vacuo to obtain (R)-2-methyl-N-(pyridin-2-ylmethyl) butan-1-amine (460 mg, crude) as a yellow oil.

LCMS (ESI): [M+H]⁺m/z: calcd 179.16; found 179.2; Rt=0.607 min.

### Step 3: (R)-2,2,2-trifluoroethyl 2-((2-methylbutyl)(pyridin-2-ylmethyl)amino)-2-oxoacetate

(R)-2-methyl-N-(pyridin-2-ylmethyl) butan-1-amine (460 mg, 2.58 mmol) and Triethylamine (313.32 mg, 3.10 mmol, 431.57 μL) were dissolved in DCM (9.71 mL) and the resulting mixture was cooled to 25° C. in an ice/methanol bath. A solution of 2,2,2-trifluoroethyl 2-chloro-2-oxo-acetate (516.14 mg, 2.71 mmol, 351.12 μL) in DCM (1.94 mL) was added dropwise at 25° C. After the addition was completed, the resulting mixture was allowed to warm to room temperature and stirred overnight. Water (15 mL) was added to the reaction mixture and an organic layer was separated. The aqueous layer was extracted with DCM (25 mL) and combined organic layers were dried over anhydrous sodium sulfate, filtered and concentrated in vacuo to obtain (R)-2,2,2-trifluoroethyl 2-((2-methylbutyl)(pyridin-2-ylmethyl)amino)-2-oxoacetate (762 mg, crude) as a yellow oil.

LCMS (ESI): [M+H]⁺m/z: calcd 333.14; found 333.2; Rt=1.117 min.

### Step 4: (R)—N¹-(2-methylbutyl)-N1-(pyridin-2-ylmethyl)oxalamide

A solution of (R)-2,2,2-trifluoroethyl 2-((2-methylbutyl)(pyridin-2-ylmethyl)amino)-2-oxoacetate (762 mg, 2.29 mmol) in NH3/MeOH (25 mL) was stirred at 25° C. for 16 hr. The solvent was evaporated to obtain (R)—N¹-(2-methylbutyl)-N1-(pyridin-2-ylmethyl)oxalamide (578 mg, crude) as a brown gum.

LCMS (ESI): [M+H]⁺m/z: calcd 250.16; found 250.2; Rt=0.757 min.

### Step 5: N1-(4-amino-2-(tetrahydro-2H-pyran-2-yl)-2H-pyrazolo[4,3-c]pyridin-7-yl)-N2-((R)-2-methylbutyl)-N2-(pyridin-2-ylmethyl)oxalamide

To an 8 ml vial (R)—N¹-(2-methylbutyl)-N1-(pyridin-2-ylmethyl)oxalamide (150 mg, 601.67 μmol), 7-bromo-2-tetrahydropyran-2-yl-pyrazolo[4,3-c]pyridin-4-amine (196.66 mg, 661.83 μmol), Copper (1.91 mg, 30.08 μmol), Copper (I) iodide (57.29 mg, 300.83 μmol, 10.19 μL), rac-(1R,2R)—N1,N2-dimethylcyclohexane-1,2-diamine (64.19 mg, 451.25 μmol), Cesium carbonate (392.07 mg, 1.20 mmol) and Dioxane (3 mL) were charged and the resulting mixture was splurged with argon for 5 min. The vial was sealed and heated at 110° C. for 38 hr. The reaction mixture was cooled, diluted with MeOH (5 mL), and filtered. The filtrate was concentrated in vacuo. The residue was purified by HPLC (0-2-10 min 23-40-70% H₂O/MeOH/0.1NH4OH, flow 30 mL/min (loading pump 4 mL MeOH, target mass 465; column: Chromatorex SMB100-5T C18 100×19 mm, 5 microM) to obtain N1-(4-amino-2-(tetrahydro-2H-pyran-2-yl)-2H-pyrazolo[4,3-c]pyridin-7-yl)-N2-((R)-2-methylbutyl)-N2-(pyridin-2-ylmethyl)oxalamide (28.4 mg, 61.00 μmol, 10.14% yield) as a yellow gum.

LCMS (ESI): [M+H]⁺m/z: calcd 466.29; found 466.2; Rt=1.158 min.

### Step 6: The synthesis of (R)—N¹-(4-amino-1H-pyrazolo[4,3-c]pyridin-7-yl)-N2-(2-methylbutyl)-N2-(pyridin-2-ylmethyl)oxalamide

N1-(4-amino-2-(tetrahydro-2H-pyran-2-yl)-2H-pyrazolo[4,3-c]pyridin-7-yl)-N2-((R)-2-methylbutyl)-N2-(pyridin-2-ylmethyl)oxalamide (28.4 mg, 61.00 μmol) was dissolved in Methanol (1 mL) and Dioxane/HCl (1 mL) was added thereto. The resulting solution was stirred for 1 hr and the reaction mixture was concentrated in vacuo. The residue was purified by HPLC (0-2-10 min 0-85% H₂O/ACN/0.1FA flow 30 mL/min (loading pump 4 mL ACN), COLUMN: Chromatorex C18 SMB 100-5T 100×19 mm, 5 microM) to obtain (R)—N¹-(4-amino-1H-pyrazolo[4,3-c]pyridin-7-yl)-N2-(2-methylbutyl)-N2-(pyridin-2-ylmethyl)oxalamide (11.4 mg, 25.67 μmol, 43.72% yield, HCOOH) as a light-yellow gum.

¹H NMR (600 MHz, DMSO-d₆) δ 0.72-0.82 (m, 3H), 0.83-0.86 (m, 3H), 1.00-1.12 (m, 1H), 1.31-1.41 (m, 1H), 1.69-1.81 (m, 1H), 3.12-3.21 (m, 1H), 3.42-3.54 (m, 1H), 4.61-4.78 (m, 1H), 4.83-4.98 (m, 1H), 6.60-6.79 (m, 2H), 7.26-7.31 (m, 1H), 7.34-7.44 (m, 1H), 7.61-7.75 (m, 1H), 7.74-7.84 (m, 1H), 8.13-8.20 (m, 1H), 8.48-8.57 (m, 1H), 10.32-10.47 (m, 1H), 12.59-12.83 (m, 1H).

LCMS (ESI): [M+H]⁺m/z: calcd 382.22; found 382.0; Rt=0.902 min.

### Example 77. The synthesis of(S)-N1-(4-amino-1H-pyrazolo[4,3-c]pyridin-7-yl)-N2-(2-methylbutyl)-N2-(pyridin-2-ylmethyl)oxalamide (Compound 128)

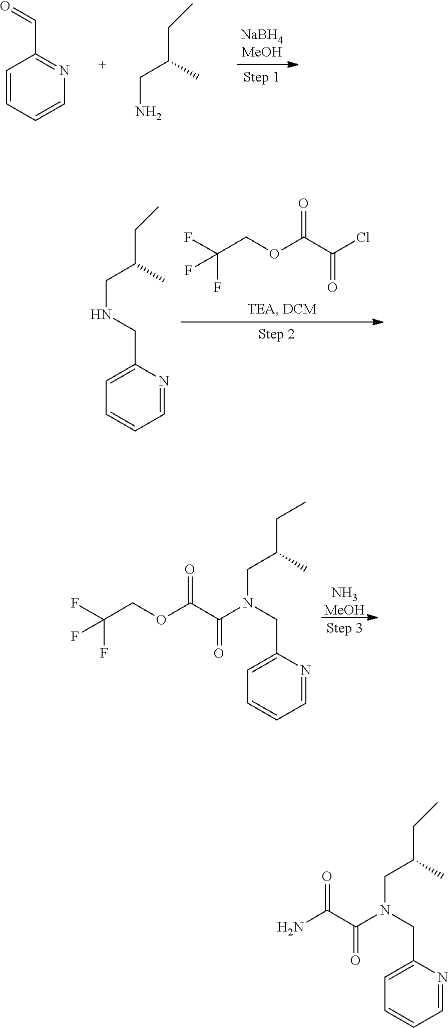

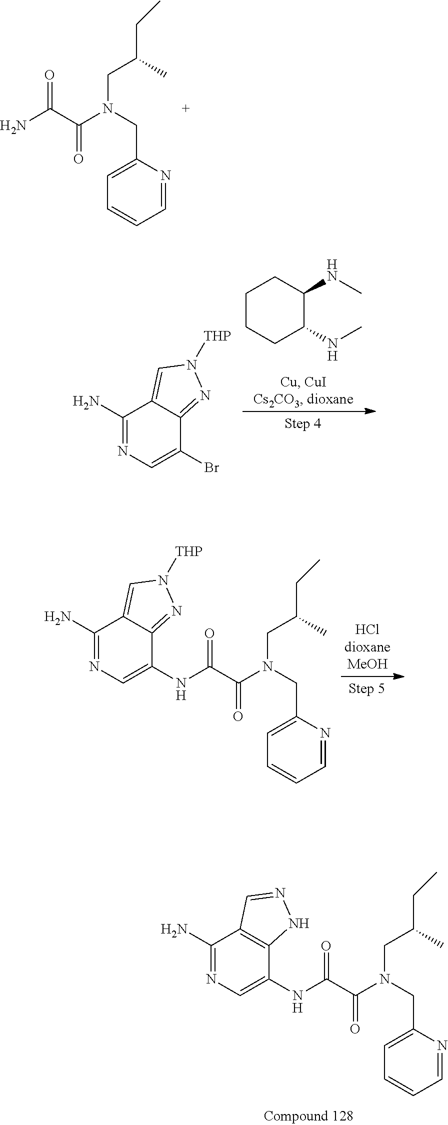

### Step 1: (S)-2-methyl-N-(pyridin-2-ylmethyl) butan-1-amine

(S)-2-methylbutan-1-amine (467.92 mg, 5.37 mmol) were added to the solution of pyridine-2-carbaldehyde (575 mg, 5.37 mmol, 511.57 μL) in MeOH (10 mL). The resulting mixture was stirred at 60° C. for 1 hour before Sodium Borohydride (406.17 mg, 10.74 mmol, 378.18 μL) was added portions thereto. After that, stirring was continued for 16 hr. Then, solvent was removed under reduced pressure and residue was partitioned between 10% aq. K₂CO₃ solution (20 mL) and DCM (20 mL). Organic layer was separated, dried over anhydrous sodium sulfate and concentrated under reduced pressure to afford(S)-2-methyl-N-(pyridin-2-ylmethyl) butan-1-amine (0.9 g, 5.05 mmol, 94.04% yield) as a yellow oil.

LCMS (ESI): [M+H]⁺m/z: calcd 179.16; found 179.2; Rt=0.619 min.

### Step 2: (S)-2,2,2-trifluoroethyl 2-((2-methylbutyl)(pyridin-2-ylmethyl)amino)-2-oxoacetate

2,2,2-trifluoroethyl 2-chloro-2-oxo-acetate (1.29 g, 6.79 mmol) was added dropwise to a solution of(S)-2-methyl-N-(pyridin-2-ylmethyl) butan-1-amine (1.1 g, 6.17 mmol) and triethylamine (749.25 mg, 7.40 mmol, 1.03 mL) in DCM (20 mL) at 0° C. After addition was complete, cooling bath was removed and resulting mixture was allowed to warm up to 20° C. and stirred for 16 hr. Then, it was washed with water, dried over anhydrous sodium sulfate and concentrated under reduced pressure, affording(S)-2,2,2-trifluoroethyl 2-((2-methylbutyl)(pyridin-2-ylmethyl)amino)-2-oxoacetate (1.96 g, 5.90 mmol, 95.59% yield) as a brown oil which was used in the next step without further purification.

LCMS (ESI): [M+H]⁺m/z: calcd 333.14; found 333.2; Rt=1.409 min.

### Step 3: (S)-N1-(2-methylbutyl)-N1-(pyridin-2-ylmethyl)oxalamide

A solution of(S)-2,2,2-trifluoroethyl 2-((2-methylbutyl)(pyridin-2-ylmethyl)amino)-2-oxoacetate (1.96 g, 5.90 mmol) in Methanol/NH3 (5N)(30 mL) was stirred at 20° C. for 16 hr. The solvent was evaporated to obtain(S)-N1-(2-methylbutyl)-N1-(pyridin-2-ylmethyl)oxalamide (0.9 g, 3.61 mmol, 61.21% yield) as a brown oil.

LCMS (ESI): [M+H]⁺m/z: calcd 250.16; found 250.0; Rt=0.752 min.

### Step 4: N1-(4-amino-2-(tetrahydro-2H-pyran-2-yl)-2H-pyrazolo[4,3-c]pyridin-7-yl)-N2-((S)-2-methylbutyl)-N2-(pyridin-2-ylmethyl)oxalamide

(S)-N1-(2-methylbutyl)-N1-(pyridin-2-ylmethyl)oxalamide (0.13 g, 521.44 μmol), 7-bromo-2-tetrahydropyran-2-yl-pyrazolo[4,3-c]pyridin-4-amine (185.94 mg, 625.73 μmol), Copper (I) iodide (19.86 mg, 104.29 μmol, 3.53 μL), Cesium carbonate (339.79 mg, 1.04 mmol) and (1R,2R)—N1,N2-dimethylcyclohexane-1,2-diamine (89.00 mg, 625.73 μmol) were mixed in dioxane (4 mL) under argon, and then stirred overnight at 100° C. for 36 hr in vial. The reaction mixture was filtered and the filtrate was concentrated in vacuo and the residue was purified by HPLC (0-2-10 min 2-30-55% H₂O/MeOH/0.1NH4OH, flow 30 mL/min ((loading pump 4 mL MeOH) target mass 465 column: XBridge BEH C18 100×19 mm, 5 microM) to give N1-(4-amino-2-(tetrahydro-2H-pyran-2-yl)-2H-pyrazolo[4,3-c]pyridin-7-yl)-N2-((S)-2-methylbutyl)-N2-(pyridin-2-ylmethyl)oxalamide (50.4 mg, 108.26 μmol, 20.76% yield) as a yellow solid.

LCMS (ESI): [M+H]⁺m/z: calcd 466.29; found 467.0; Rt=1.063 min.

### Step 5: The synthesis of(S)-N1-(4-amino-1H-pyrazolo[4,3-c]pyridin-7-yl)-N2-(2-methylbutyl)-N2-(pyridin-2-ylmethyl)oxalamide

To a solution of N1-(4-amino-2-(tetrahydro-2H-pyran-2-yl)-2H-pyrazolo[4,3-c]pyridin-7-yl)-N2-((S)-2-methylbutyl)-N2-(pyridin-2-ylmethyl)oxalamide (50.4 mg, 108.26 μmol) in MeOH (3.00 mL) was added Hydrogen chloride solution 4.0M in dioxane (800.00 mg, 21.94 mmol, 1.00 mL) at 20° C. The resulting mixture was left to stirred for 14 hr. The resulting mixture was evaporated to dryness and then submitted to reverse phase HPLC (SYSTEM 0-2-10 min 0-80% H₂O/ACN/0.1FA flow 30 mL/min (loading pump 4 mL MeOH), target mass 381 COLUMN: Chromatorex C18 SMB 100-5T 100×19 mm, 5 microM) to afford(S)-N1-(4-amino-1H-pyrazolo[4,3-c]pyridin-7-yl)-N2-(2-methylbutyl)-N2-(pyridin-2-ylmethyl)oxalamide (22.6 mg, 52.87 μmol, 48.84% yield, HCOOH) as a beige gum.

¹H NMR (600 MHz, DMSO-d₆) δ 0.70-0.88 (m, 6H), 0.95-1.16 (m, 1H), 1.27-1.41 (m, 1H), 1.70-1.86 (m, 1H), 3.19-3.23 (m, 1H), 3.45-3.51 (m, 1H), 4.41-4.81 (m, 1H), 4.87-4.96 (m, 1H), 6.59-6.91 (m, 2H), 7.25-7.33 (m, 1H), 7.33-7.45 (m, 1H), 7.59-7.73 (m, 1H), 7.75-7.81 (m, 1H), 8.15-8.20 (m, 1H), 8.47-8.77 (m, 1H), 9.59-10.47 (m, 1H), 12.61-13.42 (m, 1H).

LCMS (ESI): [M+H]⁺m/z: calcd 382.22; found 382.0; Rt=0.736 min.

### Example 78. Synthesis of N-(4-amino-1H-pyrazolo[4,3-c]pyridin-7-yl)-N′-(1-phenylethyl)-N′-(2-pyridylmethyl)oxamide (Compound 174)

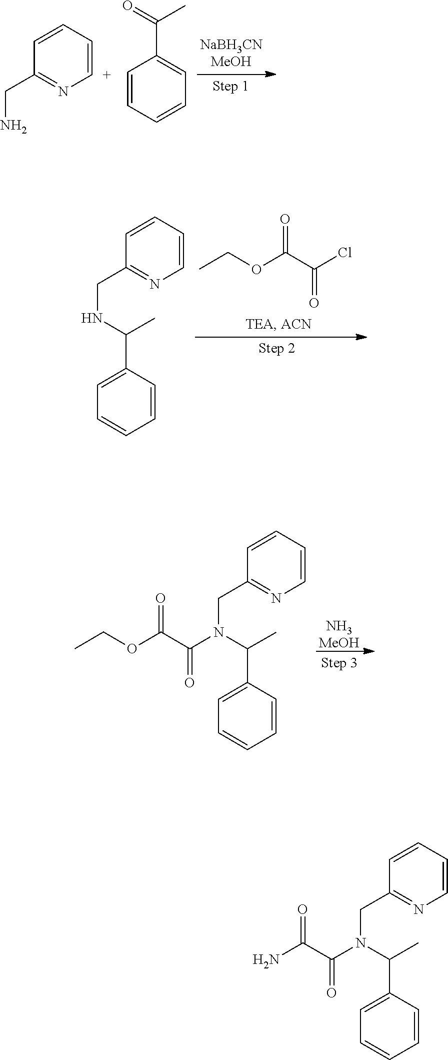

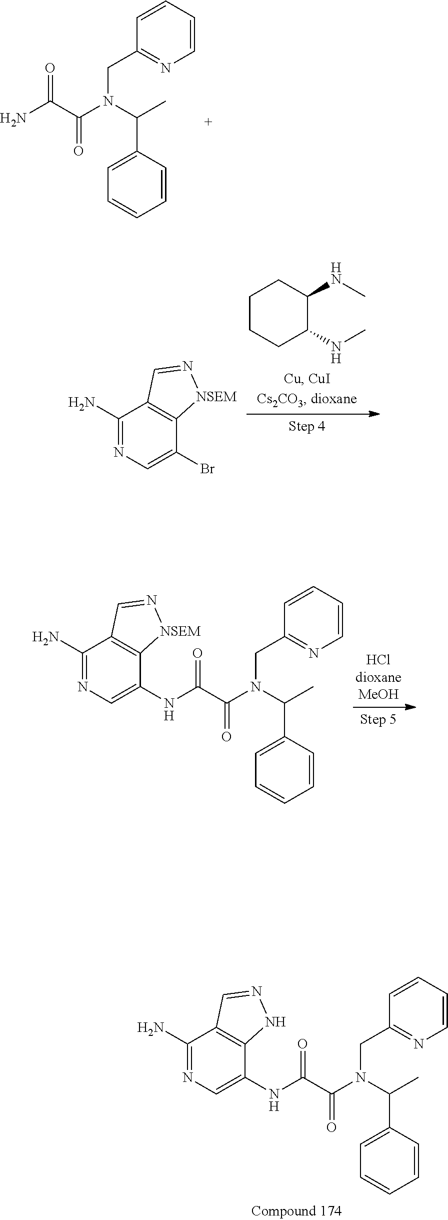

### Step 1:1-phenyl-N-(2-pyridylmethyl)ethanamine

1-phenylethanone (1 g, 8.32 mmol, 970.87 μL) and 2-pyridylmethanamine (900.06 mg, 8.32 mmol, 858.02 μL) were dissolved in MeOH (20 mL) and Sodium cyanoborohydride (784.53 mg, 12.48 mmol) was added thereto. The resulting mixture was stirred overnight. The reaction mixture was concentrated in vacuo and aq. K₂CO₃ solution (50 mL) was added to the residue. The resulting mixture was extracted with chloroform (2×50 mL) and combined organic layers were dried over anhydrous sodium sulfate, filtered and concentrated in vacuo to obtain 1-phenyl-N-(2-pyridylmethyl)ethanamine (1.3 g, crude) as a yellow oil.

LCMS (ESI): [M+H]⁺m/z: calcd 213.14; found 213.2; Rt=0.837 min.

### Step 2: ethyl 2-oxo-2-[1-phenylethyl(2-pyridylmethyl)amino]acetate

1-phenyl-N-(2-pyridylmethyl)ethanamine (1.3 g, 3.74 mmol) and TEA (755.98 mg, 7.47 mmol, 1.04 mL) were dissolved in acetonitrile (20 mL), cooled with ice-water bath and then ethyl 2-chloro-2-oxoacetate (765.02 mg, 5.60 mmol, 626.04 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-oxo-2-[1-phenylethyl(2-pyridylmethyl)amino]acetate (1.5 g, crude) as a red oil.

LCMS (ESI): [M+H]⁺m/z: calcd 313.16; found 313.2; Rt=1.142 min.

### Step 3: N′-(1-phenylethyl)-N′-(2-pyridylmethyl)oxamide

Ethyl 2-oxo-2-[1-phenylethyl(2-pyridylmethyl)amino]acetate (1.5 g, 3.36 mmol) was dissolved in MeOH (20 mL) saturated with NH3 (3.36 mmol) and stirred overnight. The RM was concentrated, redissolved in DCM, washed with water, dried over anhydrous sodium sulfate and concentrated in vacuo to give N′-(1-phenylethyl)-N′-(2-pyridylmethyl)oxamide (1 g, crude) as a brown solid.

LCMS (ESI): [M+H]⁺m/z: calcd 284.14; found 284.2; Rt=0.919 min Step 4: N-[4-amino-1-(2-trimethylsilylethoxymethyl)pyrazolo[4,3-c]pyridin-7-yl]-N′-(1-phenylethyl)-N′-(2-pyridylmethyl)oxamide

Copper (1.70 mg, 26.82 μmol), Copper (I) iodide (51.09 mg, 268.24 μmol, 9.09 μL), cesium carbonate (349.60 mg, 1.07 mmol) were added to a stirred solution of N′-(1-phenylethyl)-N′-(2-pyridylmethyl)oxamide (200 mg, 536.49 μmol), 7-bromo-1-(2-trimethylsilylethoxymethyl)pyrazolo[4,3-c]pyridin-4-amine (184.17 mg, 536.49 μmol) and rac-(1R,2R)—N1,N2-dimethylcyclohexane-1,2-diamine (148.80 mg, 1.05 mmol) in 1,4-dioxane (5 mL) under Ar atmosphere and stirred at 100° C. for 12 hr in closed vial. 90 mg of starting bromide was added additionally under argon and heated with stirring next 22 hr. Reaction mixture was filtered, solid washed with dioxane (2×3 mL), filtrate concentrated. The residue was treated with DCM (30 mL), washed with water twice, dried over anhydrous sodium sulfate and evaporated in vacuo to give N-[4-amino-1-(2-trimethylsilylethoxymethyl)pyrazolo[4,3-c]pyridin-7-yl]-N′-(1-phenylethyl)-N′-(2-pyridylmethyl)oxamide (450 mg, crude) as a brown solid.

LCMS (ESI): [M+H]⁺m/z: calcd 546.27; found 546.4; Rt=1.242 min.

### Step 5: The synthesis of N-(4-amino-1H-pyrazolo[4,3-c]pyridin-7-yl)-N′-(1-phenylethyl)-N′-(2-pyridylmethyl)oxamide

N-[4-amino-1-(2-trimethylsilylethoxymethyl)pyrazolo[4,3-c]pyridin-7-yl]-N′-(1-phenylethyl)-N′-(2-pyridylmethyl)oxamide (450 mg, 725.66 μmol) was dissolved in Dioxane (4 mL) saturated with HCl (10% by weight). MeOH (4 mL) was added for better solubility. The RM was stirred for 12 hr at 20° C. then concentrated in vacuo. The residue was dissolved in 3 mL of MeOH and subjected to HPLC (Device (Mobile Phase, Column): 2-10 min 0-15% ACN+FA) to afford N-(4-amino-1H-pyrazolo[4,3-c]pyridin-7-yl)-N′-(1-phenylethyl)-N′-(2-pyridylmethyl)oxamide (20.9 mg, 50.31 μmol, 6.93% yield) as a light-brown gum.

¹H NMR (600 MHz, dmso) δ 0.66-1.67 (m, 3H), 3.58-5.10 (m, 2H), 5.17-5.90 (m, 1H), 6.56-7.04 (m, 2H), 7.05-7.27 (m, 3H), 7.29-7.35 (m, 3H), 7.37-7.77 (m, 3H), 8.13-8.21 (m, 1H), 8.24-8.45 (m, 1H), 9.64-10.78 (m, 1H), 12.53-13.51 (m, 1H).

LCMS (ESI): [M+H]⁺m/z: calcd 416.2; found 416.2; Rt=0.884 min.

### Example 79. Synthesis of N-(4-amino-1H-pyrazolo[4,3-c]pyridin-7-yl)-N′-benzyl-N′-[1-(2-pyridyl)ethyl]oxamide (Compound 139)

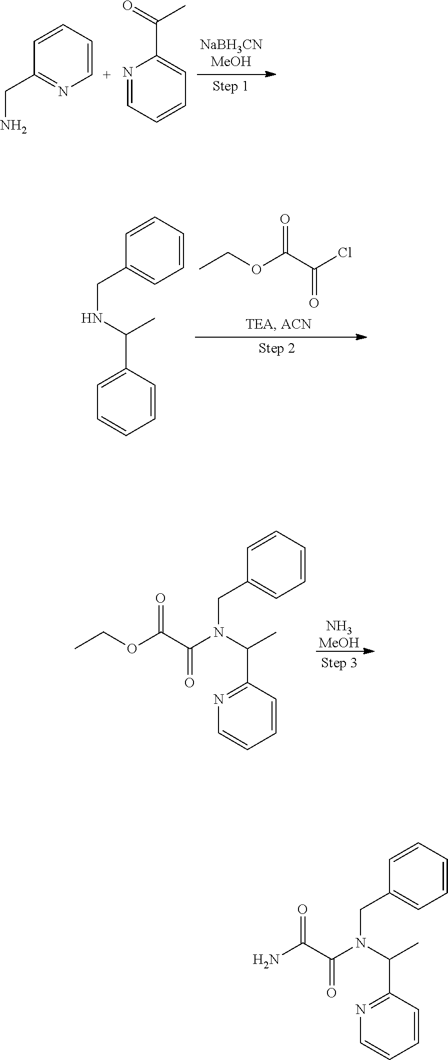

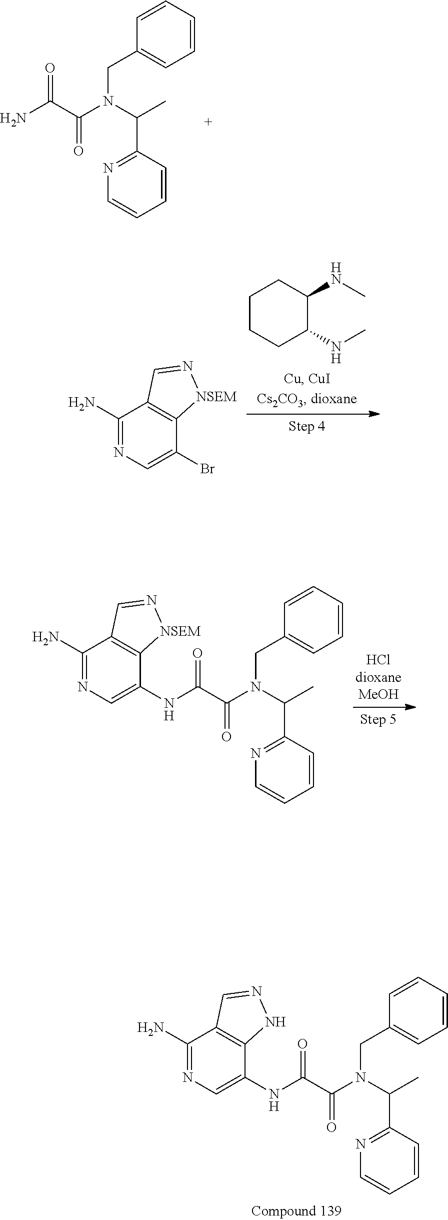

### Step 1: N-benzyl-1-(2-pyridyl)ethanamine

1-(2-pyridyl) ethanone (1 g, 8.26 mmol) and phenylmethanamine (884.56 mg, 8.26 mmol) were dissolved in MeOH (20 mL) and Sodium cyanoborohydride (778.13 mg, 12.38 mmol) was added thereto. The resulting mixture was stirred overnight. The reaction mixture was concentrated in vacuo and aq. K₂CO₃ solution (50 mL) was added to the residue. The resulting mixture was extracted with chloroform (2×50 mL) and combined organic layers were dried over anhydrous sodium sulfate, filtered and concentrated in vacuo to obtain N-benzyl-1-(2-pyridyl)ethanamine (1.7 g, crude) as a yellow oil.

LCMS (ESI): [M+H]⁺m/z: calcd 213.14; found 213.2; Rt=0.829 min.

### Step 2: ethyl 2-[benzyl-[1-(2-pyridyl)ethyl]amino]-2-oxo-acetate

N-benzyl-1-(2-pyridyl)ethanamine (1.7 g, 5.61 mmol) and TEA (1.13 g, 11.21 mmol, 1.56 mL) were dissolved in acetonitrile (20 mL), cooled with ice-water bath and then ethyl 2-chloro-2-oxoacetate (994.95 mg, 7.29 mmol, 814.20 μL) was added. Solution was stirred overnight at 20° C. The RM was concentrated in vacuo, redissolved in DCM (40 mL). and washed with water twice. Organic phase was dried over anhydrous sodium sulfate and concentrated in vacuo to give ethyl 2-[benzyl-[1-(2-pyridyl)ethyl]amino]-2-oxo-acetate (1.3 g, crude) as a yellow oil.

LCMS (ESI): [M+H]⁺m/z: calcd 313.16; found 313.2; Rt=1.349 min.

### Step 3: N′-benzyl-N′-[1-(2-pyridyl)ethyl]oxamide

Ethyl 2-[benzyl-[1-(2-pyridyl)ethyl]amino]-2-oxo-acetate (1.3 g, 2.91 mmol) was dissolved in MeOH (20 mL) saturated with NH3 (2.91 mmol) and stirred overnight. The RM was concentrated, redissolved in DCM, washed with water, dried over anhydrous sodium sulfate and concentrated in vacuo to give N′-benzyl-N′-[1-(2-pyridyl)ethyl]oxamide (1.2 g, crude) as a yellow solid.

LCMS (ESI): [M+H]⁺m/z: calcd 284.14; found 284.2; Rt=0.937 min.

### Step 4: N-[4-amino-1-(2-trimethylsilylethoxymethyl)pyrazolo[4,3-c]pyridin-7-yl]-N′-benzyl-N′-[1-(2-pyridyl)ethyl]oxamide

Copper (1.57 mg, 24.71 μmol), Copper (I) iodide (47.05 mg, 247.07 μmol, 8.37 μL), cesium carbonate (322.00 mg, 988.27 μmol) were added to a stirred solution of N′-benzyl-N′-[1-(2-pyridyl)ethyl]oxamide (200 mg, 494.13 μmol), 7-bromo-1-(2-trimethylsilylethoxymethyl)pyrazolo[4,3-c]pyridin-4-amine (169.63 mg, 494.13 μmol) and rac-(1R,2R)—N1,N2-dimethylcyclohexane-1,2-diamine (137.06 mg, 963.56 μmol) in 1,4-dioxane (5 mL) under Ar atmosphere and stirred at 100° C. for 12 hr in closed vial. Reaction mixture was filtered, solid washed with dioxane (2×3 mL), filtrate concentrated. The residue was dissolved in DCM (40 mL), washed with water twice, organic phase dried over anhydrous sodium sulfate and concentrated in vacuo to give N-[4-amino-1-(2-trimethylsilylethoxymethyl)pyrazolo[4,3-c]pyridin-7-yl]-N′-benzyl-N′-[1-(2-pyridyl)ethyl]oxamide (300 mg, crude) as a brown solid.

LCMS (ESI): [M+H]⁺m/z: calcd 546.27; found 546.0; Rt=1.253 min.

### Step 5: The synthesis of N-(4-amino-1H-pyrazolo[4,3-c]pyridin-7-yl)-N′-benzyl-N′-[1-(2-pyridyl)ethyl]oxamide

N-[4-amino-1-(2-trimethylsilylethoxymethyl)pyrazolo[4,3-c]pyridin-7-yl]-N′-benzyl-N′-[1-(2-pyridyl)ethyl]oxamide (300 mg, 192.41 μmol) was dissolved in Dioxane (4 mL) saturated with HCl (10% by weight). MeOH (4 mL) was added for better solubility. The RM was stirred for 12 hr at 20° C., then concentrated in vacuo. The residue was dissolved in 3 mL of MeOH and subjected to HPLC (Device (Mobile Phase, Column): 2-10 min 30-55% ACN flow 30 mL/min), then repurified by HPLC (Device (Mobile Phase, Column): 2-10 min 0-15% ACN+FA) to afford N-(4-amino-1H-pyrazolo[4,3-c]pyridin-7-yl)-N′-benzyl-N′-[1-(2-pyridyl)ethyl]oxamide (22.8 mg, 54.88 μmol, 28.52% yield) as a beige solid.

¹H NMR (600 MHz, dmso) δ 1.02-1.61 (m, 3H), 3.89-4.95 (m, 2H), 5.25-6.35 (m, 1H), 6.73-7.53 (m, 9H), 7.53-7.83 (m, 2H), 8.15-8.41 (m, 1H), 8.46-8.60 (m, 1H), 9.76-10.78 (m, 1H), 11.81-14.00 (m, 1H).

LCMS (ESI): [M+H]⁺m/z: calcd 416.2; found 416.4; Rt=2.263 min.

### Example 80. Synthesis of N-(4-amino-1H-pyrazolo[4,3-c]pyridin-7-yl)-N′-benzyl-N′-(pyrazol-1-ylmethyl)oxamide (Compound 181)

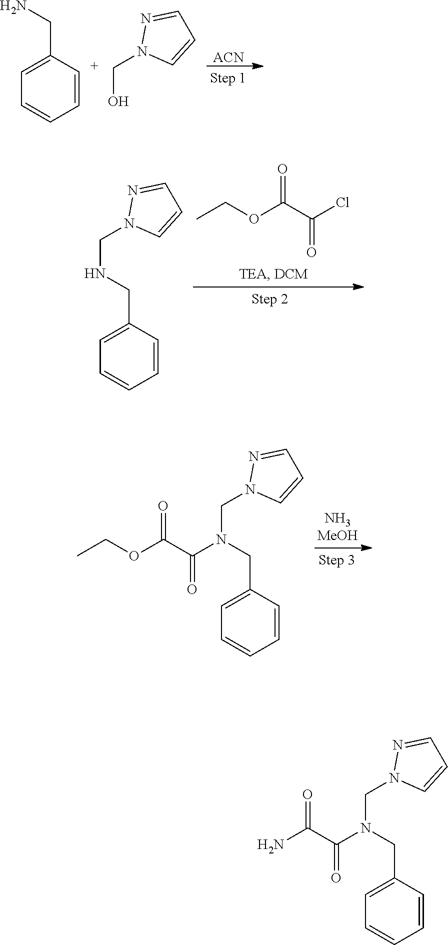

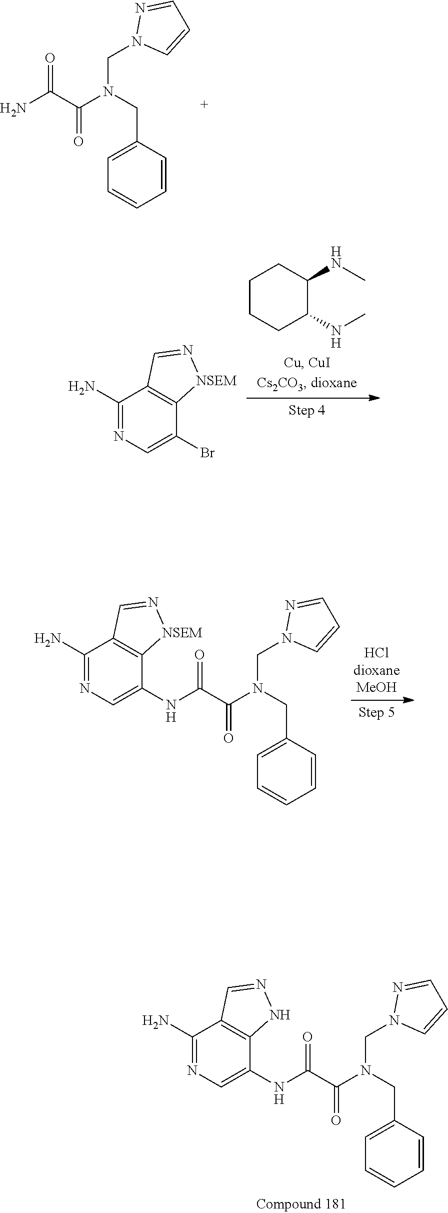

### Step 1:1-phenyl-N-(pyrazol-1-ylmethyl)methanamine

Phenylmethanamine (1.09 g, 10.19 mmol) was added to a stirred solution of pyrazol-1-ylmethanol (1 g, 10.19 mmol) in Acetonitrile (20 mL) and was stirred at 8° C. for 12 hr. The reaction mixture was evaporated in vacuo to give 1-phenyl-N-(pyrazol-1-ylmethyl)methanamine (1.1 g, 5.87 mmol, 57.63% yield) as a yellow oil.

### Step 2: ethyl 2-[benzyl(pyrazol-1-ylmethyl)amino]-2-oxo-acetate

1-phenyl-N-(pyrazol-1-ylmethyl)methanamine (1.1 g, 5.87 mmol) and TEA (594.47 mg, 5.87 mmol, 818.83 μL) dissolved in DCM (15 mL), cooled with ice-water bath and then ethyl 2-chloro-2-oxo-acetate (802.11 mg, 5.87 mmol, 656.39 μL) in DCM (20 mL) was added dropwise in 10 min. The solution was stirred overnight. The reaction mixture was washed with an aqueous solution of NaHCO₃, dried over anhydrous sodium sulfate and evaporated to give ethyl 2-[benzyl(pyrazol-1-ylmethyl)amino]-2-oxo-acetate (0.801 g, 2.79 mmol, 47.46% yield) as a yellow oil.

LCMS (ESI): [M+H]⁺m/z: calcd 288.14; found 288.2; Rt=1.320 min.

### Step 3: N′-benzyl-N′-(pyrazol-1-ylmethyl)oxamide

Ethyl 2-[benzyl(pyrazol-1-ylmethyl)amino]-2-oxo-acetate (0.801 g, 2.79 mmol) was dissolved in NH3/methanol (30 mL) solution (10% by weight) and stirred overnight. Then the reaction mixture was filtered and the clear solution was concentrated in vacuo to give N′-benzyl-N′-(pyrazol-1-ylmethyl)oxamide (0.6 g, 2.32 mmol, 83.33% yield) as a yellow gum.

LCMS (ESI): [M+H]⁺m/z: calcd 259.12; found 259.0; Rt=0.836 min.

### Step 4: N-[4-amino-1-(2-trimethylsilylethoxymethyl)pyrazolo[4,3-c]pyridin-7-yl]-N′-benzyl-N′-(pyrazol-1-ylmethyl)oxamide

N′-benzyl-N′-(pyrazol-1-ylmethyl)oxamide (0.2 g, 774.37 μmol), 7-bromo-1-(2-trimethylsilylethoxymethyl)pyrazolo[4,3-c]pyridin-4-amine (265.84 mg, 774.37 μmol), Copper (I) iodide (44.24 mg, 232.31 μmol, 7.87 μL), Cesium carbonate (504.61 mg, 1.55 mmol) and (1R,2R)—N1,N2-dimethylcyclohexane-1,2-diamine (49.57 mg, 348.47 μmol) were mixed in Dioxane (4 mL) under argon, and then stirred overnight at 100° C. for 12 hr in vial. The reaction mixture was filtered, then evaporated. The crude product was dissolved in DMSO(2 mL) and subjected to HPLC (Device (Mobile Phase, Column): 2-10 min 30-100% ACN+NH3) to give N-[4-amino-1-(2-trimethylsilylethoxymethyl)pyrazolo[4,3-c]pyridin-7-yl]-N′-benzyl-N′-(pyrazol-1-ylmethyl)oxamide (0.021 g, 40.33 μmol, 5.21% yield) as a light-yellow solid.

LCMS (ESI): [M+H]⁺m/z: calcd 521.28; found 521.2; Rt=1.267 min.

### Step 5: The synthesis of N-(4-amino-1H-pyrazolo[4,3-c]pyridin-7-yl)-N′-benzyl-N′-(pyrazol-1-ylmethyl)oxamide

N-[4-amino-1-(2-trimethylsilylethoxymethyl)pyrazolo[4,3-c]pyridin-7-yl]-N′-benzyl-N′-(pyrazol-1-ylmethyl)oxamide (0.021 g, 40.33 μmol) was dissolved in MeOH (1 mL) and diox/HCl (806.67 μmol, 1 mL) was added thereto. Then it was stirred at rt for 2 hr. The reaction mixture was evaporated. The crude product was purified by HPLC (2-10 min 20-60% methanol+FA 30 mL/min ((loading pump 4 mL methanol), column: SunFire 100×19, 5 microM) to afford N-(4-amino-1H-pyrazolo[4,3-c]pyridin-7-yl)-N′-benzyl-N′-(pyrazol-1-ylmethyl)oxamide (0.0081 g, 18.56 μmol, 46.02% yield, HCOOH) as a yellow solid.

¹H NMR (600 MHz, dmso) δ 4.56-4.89 (m, 2H), 5.52-6.04 (m, 2H), 6.29-6.38 (m, 1H), 6.86-7.11 (m, 2H), 7.24-7.36 (m, 5H), 7.52-7.73 (m, 2H), 7.78-7.99 (m, 1H), 8.19-8.31 (m, 1H), 10.57-10.83 (m, 1H), 12.99 (s, 1H).

LCMS (ESI): [M+H]⁺m/z: calcd 391.17; found 391.2; Rt=0.991 min.

### Example 81. Synthesis of N-(4-amino-1H-pyrazolo[4,3-c]pyridin-7-yl)-N′-benzyl-N′-isobutyl-oxamide (Compound 101)

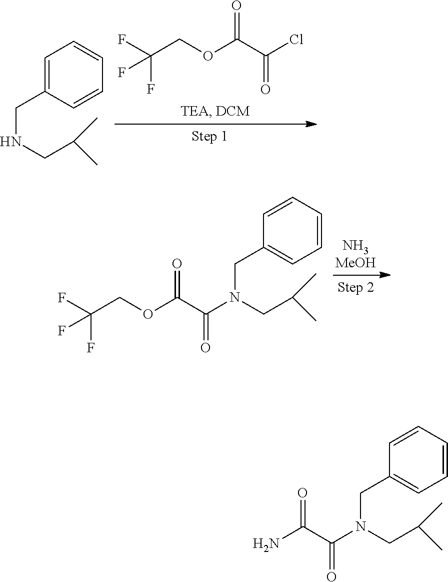

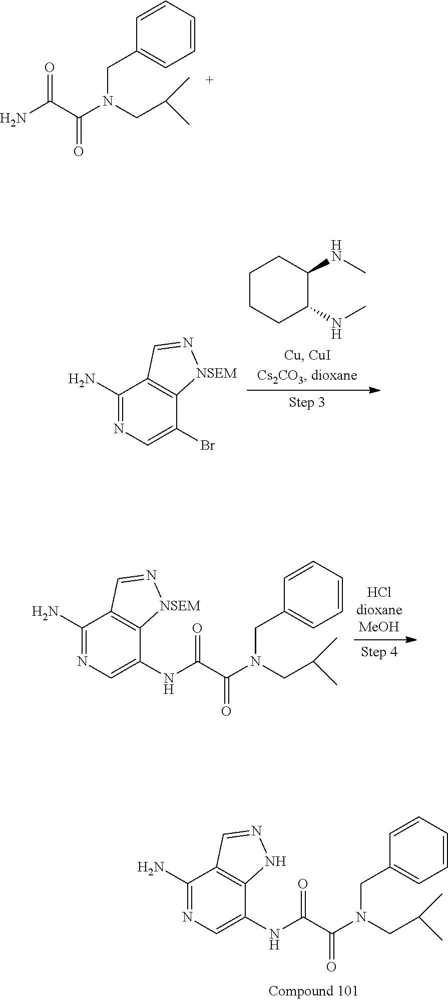

### Step 1:2,2,2-trifluoroethyl 2-[benzyl(isobutyl)amino]-2-oxo-acetate

To a solution of N-benzyl-2-methyl-propan-1-amine (0.5 g, 3.06 mmol) and TEA (371.89 mg, 3.68 mmol, 512.24 μL) in DCM (15 mL) was added 2,2,2-trifluoroethyl 2-chloro-2-oxo-acetate (641.79 mg, 3.37 mmol) at rt. After stirring at rt for 1 hr the resulting mixture was washed with water, dried, and evaporated to dryness to give 2,2,2-trifluoroethyl 2-[benzyl(isobutyl)amino]-2-oxo-acetate (0.95 g, 2.99 mmol, 97.76% yield) as a colorless gum and was used in the next step without further purification.

### Step 2: N′-benzyl-N′-isobutyl-oxamide

Ammonia (50.99 mg, 2.99 mmol) was bubbled through a solution of 2,2,2-trifluoroethyl 2-[benzyl(isobutyl)amino]-2-oxo-acetate (0.95 g, 2.99 mmol) in MeOH (25 mL) at rt. After stirring for 18 hr, the reaction mixture was evaporated to dryness to give N′-benzyl-N′-isobutyl-oxamide (0.65 g, 2.77 mmol, 92.66% yield) as a light-yellow solid.

### Step 3: N-[4-amino-1-(2-trimethylsilylethoxymethyl)pyrazolo[4,3-c]pyridin-7-yl]-N′-benzyl-N′-isobutyl-oxamide

N′-benzyl-N′-isobutyl-oxamide (150 mg, 448.16 μmol), 7-bromo-1-(2-trimethylsilylethoxymethyl)pyrazolo[4,3-c]pyridin-4-amine (153.85 mg, 448.16 μmol), Copper (I) iodide (42.68 mg, 224.08 μmol, 7.59 μL), Cesium carbonate (292.04 mg, 896.31 μmol) and (1R,2R)—N1,N2-dimethylcyclohexane-1,2-diamine (63.75 mg, 448.16 μmol) were mixed in dioxane (5.00 mL) under argon, and then stirred 72 hr at 100° C. for 18 hr in vial. The reaction mixture was filtered and the filtrate was concentrated in vacuo and the residue was purified by HPLC (40-70% 0.5-6.5 min; 30 mL/min water-acetonitrile+NH3 (loading pump 4 mL/min; acetonitrile); column XBridge C18 19×100 mm (L)) to give N-[4-amino-1-(2-trimethylsilylethoxymethyl)pyrazolo[4,3-c]pyridin-7-yl]-N′-benzyl-N′-isobutyl-oxamide (61 mg, 122.82 μmol, 27.40% yield) as a brown gum.

LCMS (ESI): [M+H]⁺m/z: calcd 497.32; found 497.2; Rt=3.801 min.

### Step 4: The synthesis of N-(4-amino-1H-pyrazolo[4,3-c]pyridin-7-yl)-N′-benzyl-N′-isobutyl-oxamide

To a solution of N-[4-amino-1-(2-trimethylsilylethoxymethyl)pyrazolo[4,3-c]pyridin-7-yl]-N′-benzyl-N′-isobutyl-oxamide (61 mg, 122.82 μmol) in MeOH (2 mL) was added Hydrogen chloride solution 4.0M in dioxane (1.60 g, 43.88 mmol, 2 mL) at 21° C. The resulting mixture was left to stir for 72 hr. The resulting mixture was evaporated to dryness and subjected to HPLC (10-35% 0.5-6.5 min; 30 mL/min water-acetonitrile+NH3 (loading pump 4 mL/min acetonitrile); column XBridge 19×100 mm (L)). N-(4-amino-1H-pyrazolo[4,3-c]pyridin-7-yl)-N′-benzyl-N′-isobutyl-oxamide (12.8 mg, 34.93 μmol, 28.44% yield) was obtained as a beige solid.

¹H NMR (600 MHz, dmso) δ 0.26-0.89 (m, 6H), 1.83-2.04 (m, 1H), 2.63-3.28 (m, 2H), 4.29-4.84 (m, 2H), 6.32-6.82 (m, 2H), 6.88-7.42 (m, 5H), 7.48-7.76 (m, 1H), 8.07-8.23 (m, 1H), 9.48-10.55 (m, 1H), 12.66-13.35 (m, 1H).

LCMS (ESI): [M+H]⁺m/z: calcd 367.21; found 367.2; Rt=1.207 min.

### Example 82. Synthesis of N-(4-amino-1H-pyrazolo[4,3-c]pyridin-7-yl)-N′-benzyl-N′-[(5-methyl-2-pyridyl)methyl]oxamide (Compound 179)

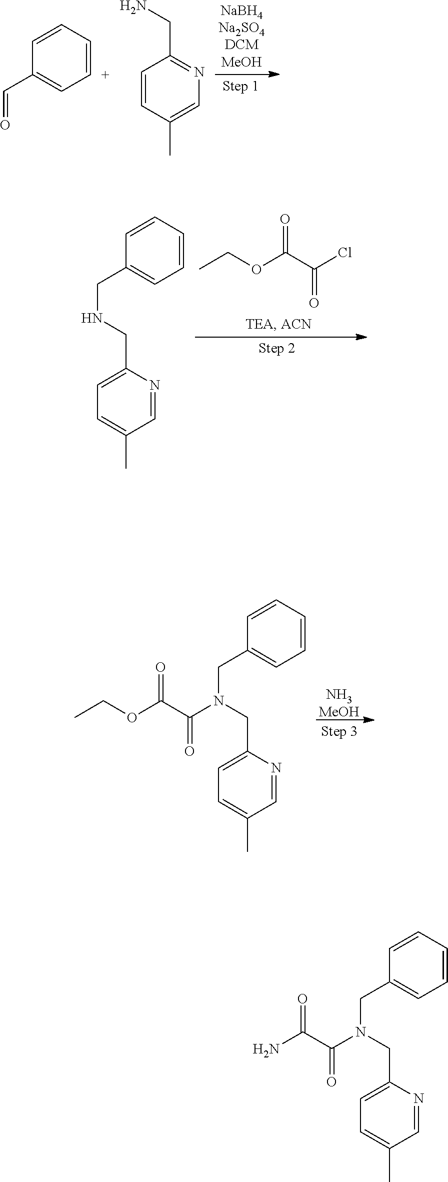

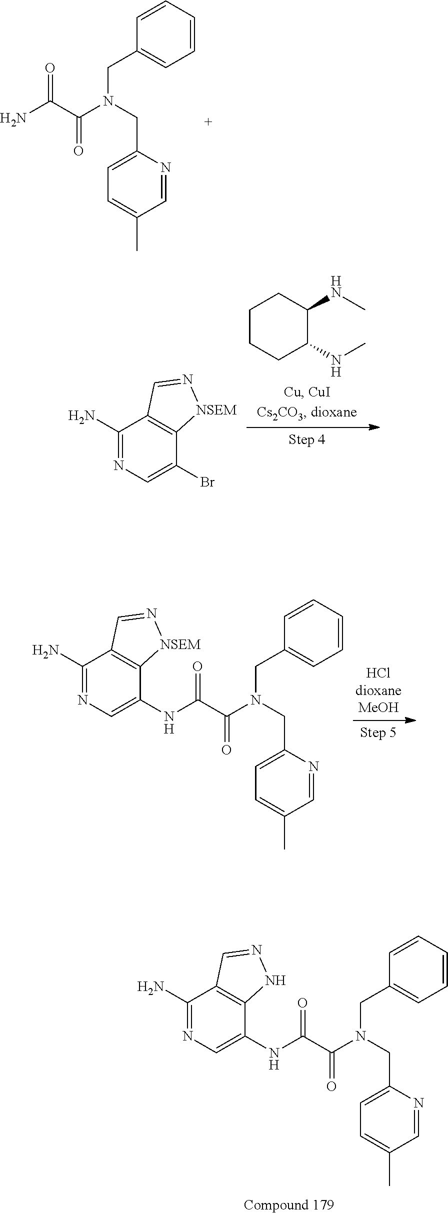

### Step 1: N-[(5-methyl-2-pyridyl)methyl]-1-phenyl-methanamine

(5-methyl-2-pyridyl)methanamine (1.00 g, 8.19 mmol), Sodium sulfate, anhydrous (1.16 g, 8.19 mmol, 433.84 μL) and benzaldehyde (868.66 mg, 8.19 mmol) were mixed in DCM (20 mL) at 20° C. The resulting mixture was stirred at 20° C. for 12 hr, then filtered and concentrated in vacuo. The residue was dissolved in methanol (20 mL), cooled to 5° and Sodium Borohydride (340.62 mg, 9.00 mmol, 317.15 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford N-[(5-methyl-2-pyridyl)methyl]-1-phenyl-methanamine (1.6 g, crude) as a brown oil.

LCMS (ESI): [M+H]⁺m/z: calcd 213.14; found 213.2; Rt=0.717 min.

### Step 2: ethyl 2-[benzyl-[(5-methyl-2-pyridyl)methyl]amino]-2-oxo-acetate

N-[(5-methyl-2-pyridyl)methyl]-1-phenyl-methanamine (1.6 g, 4.90 mmol) and TEA (991.45 mg, 9.80 mmol, 1.37 mL) were dissolved in acetonitrile (20 mL), cooled with ice-water bath and then ethyl 2-chloro-2-oxoacetate (869.54 mg, 6.37 mmol, 711.57 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-[benzyl-[(5-methyl-2-pyridyl)methyl]amino]-2-oxo-acetate (1.68 g, crude) as a brown oil.

LCMS (ESI): [M+H]⁺m/z: calcd 313.16; found 313.0; Rt=1.129 min.

### Step 3: N′-benzyl-N′-[(5-methyl-2-pyridyl)methyl]oxamide

Ethyl 2-[benzyl-[(5-methyl-2-pyridyl)methyl]amino]-2-oxo-acetate (1.68 g, 3.76 mmol) was dissolved in MeOH (20 mL) saturated with NH3 (3.76 mmol) and stirred overnight. The RM was concentrated, redissolved in DCM, washed with water, dried over anhydrous sodium sulfate and concentrated in vacuo to give N′-benzyl-N′-[(5-methyl-2-pyridyl)methyl]oxamide (1.27 g, crude) as a brown oil.

LCMS (ESI): [M+H]⁺m/z: calcd 284.14; found 284.2; Rt=0.806 min.

### Step 4: N-[4-amino-1-(2-trimethylsilylethoxymethyl)pyrazolo[4,3-c]pyridin-7-yl]-N′-benzyl-N′-[(5-methyl-2-pyridyl)methyl]oxamide

Copper (1.57 mg, 24.71 μmol), Copper (I) iodide (47.05 mg, 247.07 μmol, 8.37 μL), cesium carbonate (322.00 mg, 988.27 μmol) were added to a stirred solution of N′-benzyl-N′-[(5-methyl-2-pyridyl)methyl]oxamide (200 mg, 494.13 μmol), 7-bromo-1-(2-trimethylsilylethoxymethyl)pyrazolo[4,3-c]pyridin-4-amine (169.63 mg, 494.13 μmol) and rac-(1R,2R)—N1,N2-dimethylcyclohexane-1,2-diamine (137.06 mg, 963.56 μmol) in 1,4-dioxane (5 mL) under Ar atmosphere and stirred at 100° C. for 12 hr in closed vial. The reaction mixture was treated with water, then oily residue on the filter was dissolved in DCM (30 mL), dried over anhydrous sodium sulfate and evaporated in vacuo to give N-[4-amino-1-(2-trimethylsilylethoxymethyl)pyrazolo[4,3-c]pyridin-7-yl]-N′-benzyl-N′-[(5-methyl-2-pyridyl)methyl]oxamide (300 mg, crude) as a brown solid.

LCMS (ESI): [M+H]⁺m/z: calcd 546.27; found 546.5; Rt=0.960 min.

### Step 5: The synthesis of N-(4-amino-1H-pyrazolo[4,3-c]pyridin-7-yl)-N′-benzyl-N′-[(5-methyl-2-pyridyl)methyl]oxamide

N-[4-amino-1-(2-trimethylsilylethoxymethyl)pyrazolo[4,3-c]pyridin-7-yl]-N′-benzyl-N′-[(5-methyl-2-pyridyl)methyl]oxamide (300 mg, 280.37 μmol) was dissolved in Dioxane (4 mL) saturated with HCl (10% by weight). MeOH (4 mL) was added for better solubility. The RM was stirred for 12 hr at 20° C. then concentrated in vacuo. The residue was dissolved in 3 mL of MeOH and subjected to HPLC (Device (Mobile Phase, Column): 2-10 min 45-60% water-methanol+NH3), then repurified by HPLC (Device (Mobile Phase, Column): 2-10 min 20-50% MeOH+FA flow 30 mL/min) to afford N-(4-amino-1H-pyrazolo[4,3-c]pyridin-7-yl)-N′-benzyl-N′-[(5-methyl-2-pyridyl)methyl]oxamide (8.5 mg, 18.42 μmol, 6.57% yield, HCOOH) as a yellow solid.

¹H NMR (600 MHz, dmso) δ 2.24-2.29 (m, 3H), 4.35-4.57 (m, 2H), 4.57-4.87 (m, 2H), 6.53-7.01 (m, 2H), 7.21-7.37 (m, 6H), 7.53-7.69 (m, 2H), 8.15-8.23 (m, 1H), 8.25-8.38 (m, 1H), 9.76-10.61 (m, 1H), 12.67-13.38 (m, 1H).

LCMS (ESI): [M+H]⁺m/z: calcd 416.2; found 417.2; Rt=1.938 min.

### Example 83. Synthesis of N-(4-amino-1H-pyrazolo[4,3-c]pyridin-7-yl)-N′-isobutyl-N′-(2-pyridylmethyl)oxamide (Compound 160)

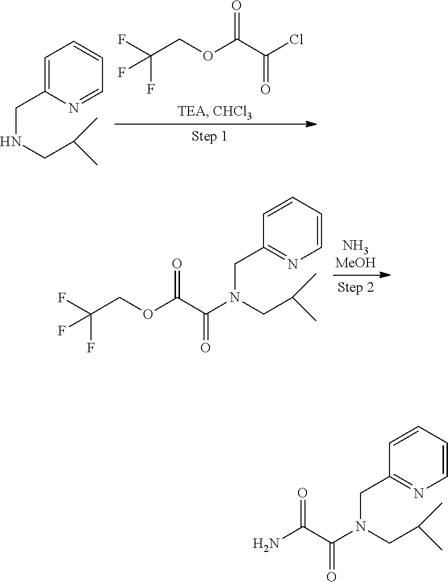

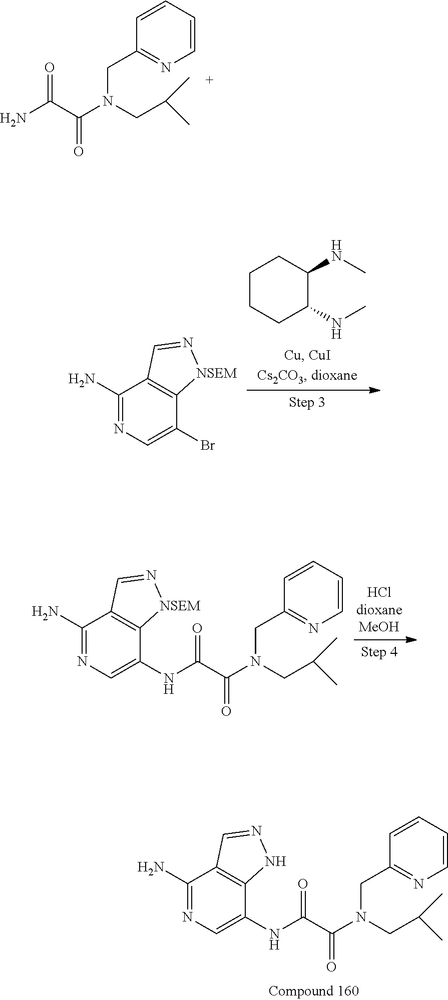

### Step 1:2,2,2-trifluoroethyl 2-[isobutyl(2-pyridylmethyl)amino]-2-oxo-acetate

To a stirred solution of 2-methyl-N-(2-pyridylmethyl)propan-1-amine (0.5 g, 3.04 mmol) and Triethylamine (435.60 mg, 4.30 mmol, 0.6 mL) in CHCl₃ (10 mL) was added 2,2,2-trifluoroethyl 2-chloro-2-oxo-acetate (0.7 g, 3.67 mmol). The resulting reaction mixture was stirred at 25° C. for 16 hr. Upon completion, the reaction mixture was evaporated, quenched with water (20 mL). The aqueous phase was extracted with CHCl₃ (2×20 mL). The combined organic phase was dried over anhydrous sodium sulfate and concentrated under reduced pressure. The reaction was successful. The desired product 2,2,2-trifluoroethyl 2-[isobutyl(2-pyridylmethyl)amino]-2-oxo-acetate (0.9 g, 2.83 mmol, 92.88% yield) was isolated as a yellow oil.

LCMS (ESI): [M+H]⁺m/z: calcd 319.13; found 319.2; Rt=1.067 min.

### Step 2: N′-isobutyl-N′-(2-pyridylmethyl)oxamide

To a stirred solution of 2,2,2-trifluoroethyl 2-[isobutyl(2-pyridylmethyl)amino]-2-oxo-acetate (0.9 g, 2.83 mmol) in MeOH (10 mL) was added NH3/MeOH (5 mL). The resulting reaction mixture was stirred at 25° C. for 6 hr. Upon completion, the reaction mixture was concentrated under reduced pressure. The reaction was successful. The desired product N′-isobutyl-N′-(2-pyridylmethyl)oxamide (0.6 g, 2.55 mmol, 90.19% yield) was isolated as a yellow solid.

LCMS (ESI): [M+H]⁺m/z: calcd 236.14; found 236.2; Rt=0.846 min.

### Step 3: N-[4-amino-1-(2-trimethylsilylethoxymethyl)pyrazolo[4,3-c]pyridin-7-yl]-N′-isobutyl-N′-(2-pyridylmethyl)oxamide

N′-isobutyl-N′-(2-pyridylmethyl)oxamide (0.15 g, 637.53 μmol), 7-bromo-1-(2-trimethylsilylethoxymethyl)pyrazolo[4,3-c]pyridin-4-amine (0.22 g, 640.85 μmol), Cu(40 mg, 629.43 μmol), Copper (I) iodide (120 mg, 630.09 μmol, 21.35 μL) and Cesium carbonate (0.4 g, 1.23 mmol) were mixed together in Dioxane (5 mL). The resulting suspension was degassed with argon at 25° C. for 0.1 hr. (1R,2R)—N1,N2-dimethylcyclohexane-1,2-diamine (180.00 mg, 1.27 mmol, 0.2 mL) was added thereto and the resulting mixture was stirred for 16 hr at 100° C. Upon completion, the reaction mixture was concentrated under reduced pressure, dissolved in CHCl₃, dried over anhydrous sodium sulfate, filtered through layer of silica and the filtrate was evaporated in vacuo. The reaction was successful. The desired product N-[4-amino-1-(2-trimethylsilylethoxymethyl)pyrazolo[4,3-c]pyridin-7-yl]-N′-isobutyl-N′-(2-pyridylmethyl)oxamide (0.15 g, 301.41 μmol, 47.28% yield) was isolated as a yellow oil.

LCMS (ESI): [M+H]⁺m/z: calcd 498.27; found 498.2; Rt=1.122 min.

### Step 4: The synthesis of N-(4-amino-1H-pyrazolo[4,3-c]pyridin-7-yl)-N′-isobutyl-N′-(2-pyridylmethyl)oxamide

To a stirred solution of N-[4-amino-1-(2-trimethylsilylethoxymethyl)pyrazolo[4,3-c]pyridin-7-yl]-N′-isobutyl-N′-(2-pyridylmethyl)oxamide (0.15 g, 301.41 μmol) in MeOH (4 mL) was added Dioxane/HCl (2 mL). The resulting reaction mixture was stirred at 25° C. for 3h. Upon completion, the reaction mixture was concentrated under reduced pressure. The obtained crude product was purified by reverse phase HPLC chromatography (Device (Mobile Phase, Column): SYSTEM 0-0-30% 0-1-6 min H₂O/ACN/0.1% FA, flow: 30 mL/min (loading pump 4 mL/min ACN) target mass 367 column: XBridge BEH C18 5 μm 130A) to afford product N-(4-amino-1H-pyrazolo[4,3-c]pyridin-7-yl)-N′-isobutyl-N′-(2-pyridylmethyl)oxamide (8 mg, 19.35 μmol, 6.42% yield, HCOOH) as a white solid.

¹H NMR (600 MHz, dmso) 8 0.81-0.92 (m, 6H), 1.94-2.07 (m, 1H), 3.16-3.46 (m, 2H), 4.65-4.96 (m, 2H), 6.58-6.72 (m, 2H), 7.27-7.32 (m, 1H), 7.34-7.46 (m, 1H), 7.59-7.74 (m, 1H), 7.74-7.82 (m, 1H), 8.15-8.23 (m, 1H), 8.44-8.61 (m, 1H), 10.28-10.52 (m, 1H), 12.62-12.86 (m, 1H).

LCMS (ESI): [M+H]⁺m/z: calcd 368.2; found 368.2; Rt=1.337 min.

### Example 84. Synthesis of N-(4-amino-1H-pyrazolo[4,3-c]pyridin-7-yl)-N′-benzyl-N′-[(3-methyl-2-pyridyl)methyl]oxamide (Compound 114)

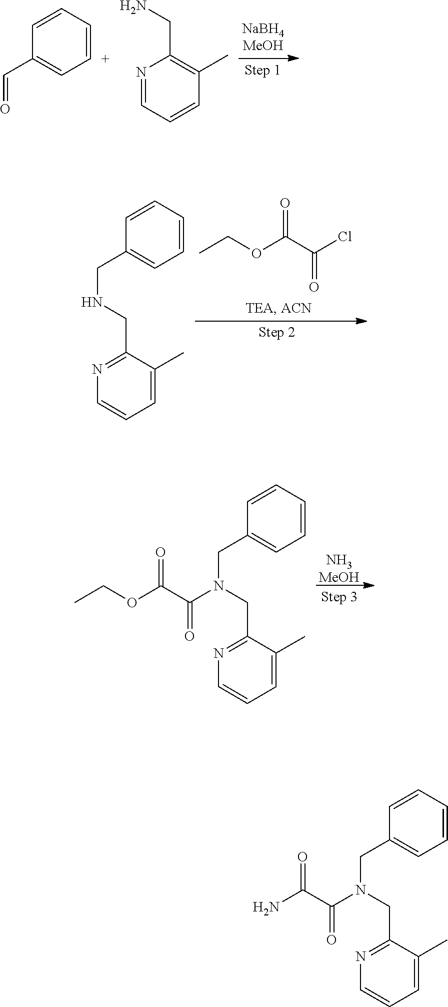

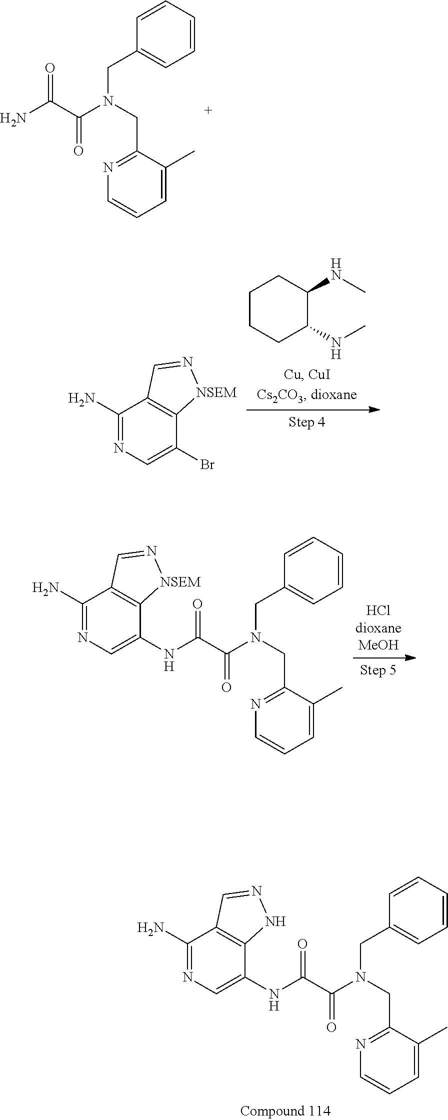

### Step 1: N-[(3-methyl-2-pyridyl)methyl]-1-phenyl-methanamine

(3-methyl-2-pyridyl)methanamine (2 g, 16.37 mmol) and benzaldehyde (1.74 g, 16.37 mmol) were mixed in methanol (20 mL) at 20° C. The resulting mixture was stirred at 20° C. for 12 hr, then cooled to 0° C. and Sodium Borohydride (681.25 mg, 18.01 mmol, 634.31 μL) was added. The reaction mixture was allowed to warm to 20° C. and stirred for 1 hr, then concentrated in vacuo. The residue was diluted with water (25 mL) and extracted with DCM (40 mL). The organic layer was separated, dried over anhydrous sodium sulfate and concentrated in vacuo to afford N-[(3-methyl-2-pyridyl)methyl]-1-phenyl-methanamine (3.4 g, crude) as a brown oil.

LCMS (ESI): [M+H]⁺m/z: calcd 213.14; found 213.0; Rt=0.539 min.

### Step 2: ethyl 2-[benzyl-[(3-methyl-2-pyridyl)methyl]amino]-2-oxo-acetate

N-[(3-methyl-2-pyridyl)methyl]-1-phenyl-methanamine (3.4 g, 4.80 mmol) and TEA (1.46 g, 14.41 mmol, 2.01 mL) were dissolved in acetonitrile (20 mL), cooled with ice-water bath and then ethyl 2-chloro-2-oxo-acetate (984.02 mg, 7.21 mmol, 805.25 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-[benzyl-[(3-methyl-2-pyridyl)methyl]amino]-2-oxo-acetate (3.45 g, crude) as a brown oil.

LCMS (ESI): [M+H]⁺m/z: calcd 313.16; found 313.2; Rt=0.986 min.

### Step 3: N′-benzyl-N′-[(3-methyl-2-pyridyl)methyl]oxamide

Ethyl 2-[benzyl-[(3-methyl-2-pyridyl)methyl]amino]-2-oxo-acetate (3.45 g, 2.76 mmol) was dissolved in MeOH (20 mL), saturated with NH3 (2.76 mmol) and stirred overnight. The RM was concentrated, redissolved in DCM, washed with water, dried over anhydrous sodium sulfate, and concentrated in vacuo to give crude material (2.35 g). Part of this amide (0.9 g) was subjected to HPLC (2-10 min 10-30% ACN+FA 30 mL/min) to afford N′-benzyl-N′-[(3-methyl-2-pyridyl)methyl]oxamide (180 mg, 635.31 μmol, 23.01% yield) as a brown solid.

LCMS (ESI): [M+H]⁺m/z: calcd 284.16; found 284.2; Rt=0.722 min.

### Step 4: N-[4-amino-1-(2-trimethylsilylethoxymethyl)pyrazolo[4,3-c]pyridin-7-yl]-N′-benzyl-N′-[(3-methyl-2-pyridyl)methyl]oxamide

Copper (1.41 mg, 22.24 μmol), Copper (I) iodide (42.35 mg, 222.36 μmol, 7.54 μL), cesium carbonate (289.80 mg, 889.44 μmol) were added to a stirred solution of N′-benzyl-N′-[(3-methyl-2-pyridyl)methyl]oxamide (180 mg, 444.72 μmol), 7-bromo-1-(2-trimethylsilylethoxymethyl)pyrazolo[4,3-c]pyridin-4-amine (152.67 mg, 444.72 μmol) and rac-(1R,2R)—N1,N2-dimethylcyclohexane-1,2-diamine (123.35 mg, 867.20 μmol) in 1,4-dioxane (5 mL) under Ar atmosphere and stirred at 100° C. for 12 hr in closed vial. The reaction mixture was treated with water, then oily residue on the filter was dissolved in DCM (30 mL), dried over anhydrous sodium sulfate and evaporated in vacuo to give N-[4-amino-1-(2-trimethylsilylethoxymethyl)pyrazolo[4,3-c]pyridin-7-yl]-N′-benzyl-N′-[(3-methyl-2-pyridyl)methyl]oxamide (165 mg, crude) as a brown oil.

LCMS (ESI): [M+H]⁺m/z: calcd 546.27; found 546.2; Rt=1.254 min.

### Step 5: The synthesis of N-(4-amino-1H-pyrazolo[4,3-c]pyridin-7-yl)-N′-benzyl-N′-[(3-methyl-2-pyridyl)methyl]oxamide

N-[4-amino-1-(2-trimethylsilylethoxymethyl)pyrazolo[4,3-c]pyridin-7-yl]-N′-benzyl-N′-[(3-methyl-2-pyridyl)methyl]oxamide (169.04 mg, 151.78 μmol) was dissolved in Dioxane (4 mL) saturated with HCl (10% by weight). MeOH (4 mL) was added for better solubility. The RM was stirred for 12 hr at 20° C., then concentrated in vacuo. The residue was dissolved in 3 mL of MeOH and subjected to HPLC (Device (Mobile Phase, Column): 2-10 min 45-60% water-methanol+NH3), then repurified by HPLC (Device (Mobile Phase, Column): 2-10 min 20-50% MeOH+FA flow 30 mL/min) to afford N-(4-amino-1H-pyrazolo[4,3-c]pyridin-7-yl)-N′-benzyl-N′-[(3-methyl-2-pyridyl)methyl]oxamide (16.8 mg, 36.41 μmol, 23.99% yield, HCOOH) as a yellow solid.

¹H NMR (600 MHz, dmso) δ 2.01-2.26 (m, 3H), 4.48-4.69 (m, 2H), 4.88-5.02 (m, 2H), 6.55-6.72 (m, 2H), 7.14-7.24 (m, 1H), 7.24-7.38 (m, 5H), 7.46-7.58 (m, 1H), 7.59-7.69 (m, 1H), 8.13-8.19 (m, 1H), 8.34-8.43 (m, 1H), 10.31-10.57 (m, 1H), 12.59-12.85 (m, 1H).

LCMS (ESI): [M+H]⁺m/z: calcd 416.2; found 416.0; Rt=2.298 min.

### Example 85. Synthesis of N-(4-amino-1H-pyrazolo[4,3-c]pyridin-7-yl)-N′-(o-tolylmethyl)-N′-(2-pyridylmethyl)oxamide (Compound 105)

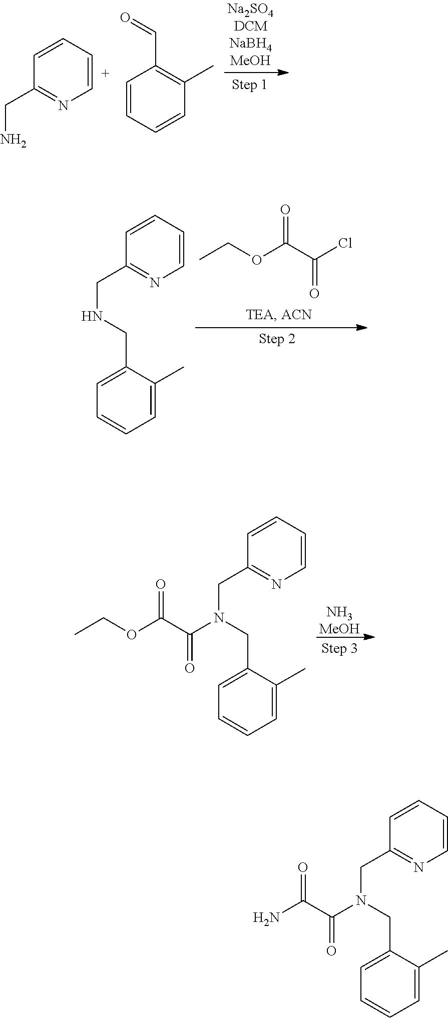

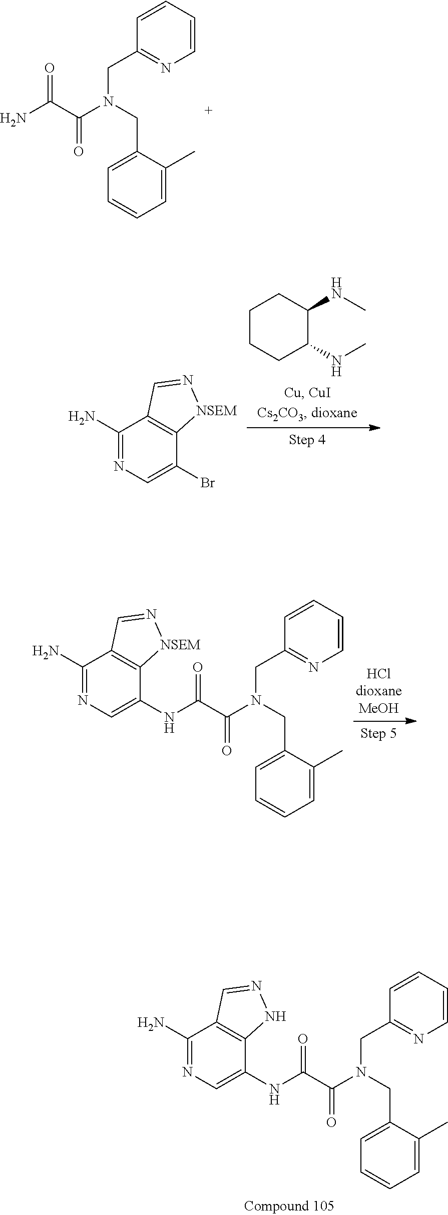

### Step 1:1-(o-tolyl)-N-(2-pyridylmethyl)methanamine

2-pyridylmethanamine (1 g, 9.25 mmol, 953.29 μL), Sodium sulfate, anhydrous (19.70 g, 138.71 mmol, 7.35 mL) and 2-methylbenzaldehyde (1.11 g, 9.25 mmol, 1.07 mL) were mixed in DCM (20 mL) at 20° C. The resulting mixture was stirred at 20° C. for 12 hr, then filtered and concentrated in vacuo. The residue was dissolved in methanol (20 mL), cooled to 5° C. and Sodium Borohydride (384.80 mg, 10.17 mmol, 358.29 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford 1-(o-tolyl)-N-(2-pyridylmethyl)methanamine (1.5 g, crude) as a brown oil.

LCMS (ESI): [M+H]⁺m/z: calcd 213.14; found 213.2; Rt=0.714 min.

### Step 2: ethyl 2-[o-tolylmethyl(2-pyridylmethyl)amino]-2-oxo-acetate

1-(o-tolyl)-N-(2-pyridylmethyl)methanamine (1.5 g, 4.95 mmol) and TEA (1.00 g, 9.89 mmol, 1.38 mL) were dissolved in acetonitrile (20 mL), cooled with ice-water bath and then ethyl 2-chloro-2-oxoacetate (945.43 mg, 6.92 mmol, 773.67 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-[o-tolylmethyl(2-pyridylmethyl)amino]-2-oxo-acetate (1.6 g, crude) as a brown oil.

LCMS (ESI): [M+H]⁺m/z: calcd 313.16; found 313.0; Rt=1.180 min.

### Step 3: N′-(o-tolylmethyl)-N′-(2-pyridylmethyl)oxamide

Ethyl 2-[o-tolylmethyl(2-pyridylmethyl)amino]-2-oxo-acetate (1.6 g, 3.33 mmol) was dissolved in MeOH (20 mL) saturated with NH3 (3.33 mmol) and stirred overnight. The RM was concentrated, redissolved in DCM, washed with water, dried over anhydrous sodium sulfate and concentrated in vacuo to give N′-(o-tolylmethyl)-N′-(2-pyridylmethyl)oxamide (1.2 g, crude) as a brown solid.

LCMS (ESI): [M+H]⁺m/z: calcd 284.14; found 284.2; Rt=0.872 min.

### Step 4: N-[4-amino-1-(2-trimethylsilylethoxymethyl)pyrazolo[4,3-c]pyridin-7-yl]-N′-(0-tolylmethyl)-N′-(2-pyridylmethyl)oxamide

Copper (1.46 mg, 22.94 μmol), Copper (I) iodide (43.69 mg, 229.42 μmol, 7.77 μL), cesium carbonate (299.00 mg, 917.68 μmol) were added to a stirred solution of N′-(o-tolylmethyl)-N′-(2-pyridylmethyl)oxamide (200 mg, 458.84 μmol), 7-bromo-1-(2-trimethylsilylethoxymethyl)pyrazolo[4,3-c]pyridin-4-amine (157.52 mg, 458.84 μmol) and rac-(1R,2R)—N1,N2-dimethylcyclohexane-1,2-diamine (127.27 mg, 894.73 μmol) in 1,4-dioxane (5 mL) under Ar atmosphere and stirred at 100° C. for 12 hr in closed vial. The reaction mixture was treated with water, then oily residue on the filter was dissolved in DCM (30 mL), dried over anhydrous sodium sulfate and evaporated in vacuo to give N-[4-amino-1-(2-trimethylsilylethoxymethyl)pyrazolo[4,3-c]pyridin-7-yl]-N′-(o-tolylmethyl)-N′-(2-pyridylmethyl)oxamide (270 mg, crude) as a brown oil.

LCMS (ESI): [M+H]⁺m/z: calcd 546.27; found 546.4; Rt=1.271 min.

### Step 5: The synthesis of N-(4-amino-1H-pyrazolo[4,3-c]pyridin-7-yl)-N′-(o-tolylmethyl)-N′-(2-pyridylmethyl)oxamide

N-[4-amino-1-(2-trimethylsilylethoxymethyl)pyrazolo[4,3-c]pyridin-7-yl]-N′-(o-tolylmethyl)-N′-(2-pyridylmethyl)oxamide (270 mg, 272.12 μmol) was dissolved in Dioxane (4 mL) saturated with HCl (10% by weight). MeOH (4 mL) was added for better solubility. The RM was stirred for 12 hr at 20° C., then concentrated in vacuo. The residue was dissolved in 3 mL of MeOH and subjected to HPLC (Device (Mobile Phase, Column): 2-10 min 45-60% water-methanol+NH3), then repurified by HPLC (Device (Mobile Phase, Column): 2-10 min 20-50% MeOH+FA flow 30 mL/min) to afford N-(4-amino-1H-pyrazolo[4,3-c]pyridin-7-yl)-N′-(o-tolylmethyl)-N′-(2-pyridylmethyl)oxamide (24 mg, 52.01 μmol, 19.11% yield, HCOOH) as a yellow solid.

¹H NMR (600 MHz, dmso) δ 1.81-2.22 (m, 3H), 4.48-4.63 (m, 2H), 4.81-4.93 (m, 2H), 6.66 (s, 2H), 7.10-7.30 (m, 5H), 7.33-7.40 (m, 1H), 7.58-7.66 (m, 1H), 7.73-7.84 (m, 1H), 8.15-8.30 (m, 1H), 8.45-8.56 (m, 1H), 9.73-10.61 (m, 1H), 12.72-13.42 (m, 1H).

LCMS (ESI): [M+H]⁺m/z: calcd 416.2; found 416.4; Rt=2.384 min.

### Example 86. Synthesis of N-(4-amino-1H-pyrazolo[4,3-c]pyridin-7-yl)-N′,N′-bis(2-pyridylmethyl)oxamide (Compound 89)

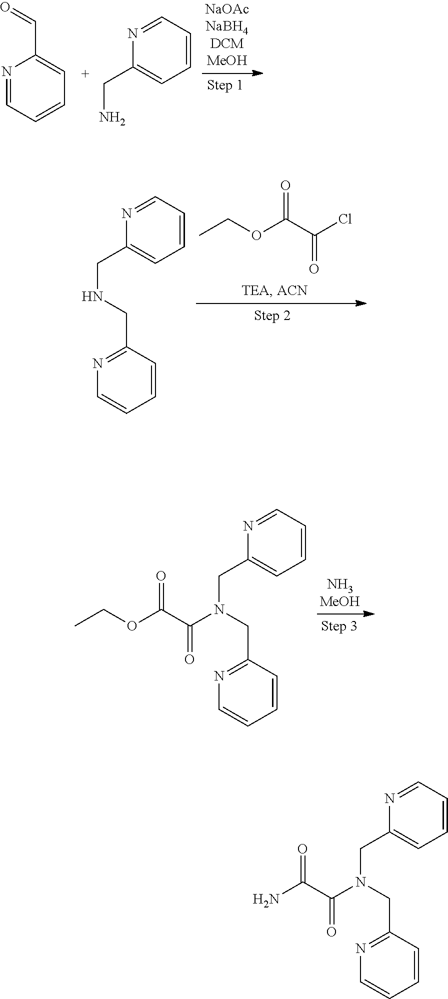

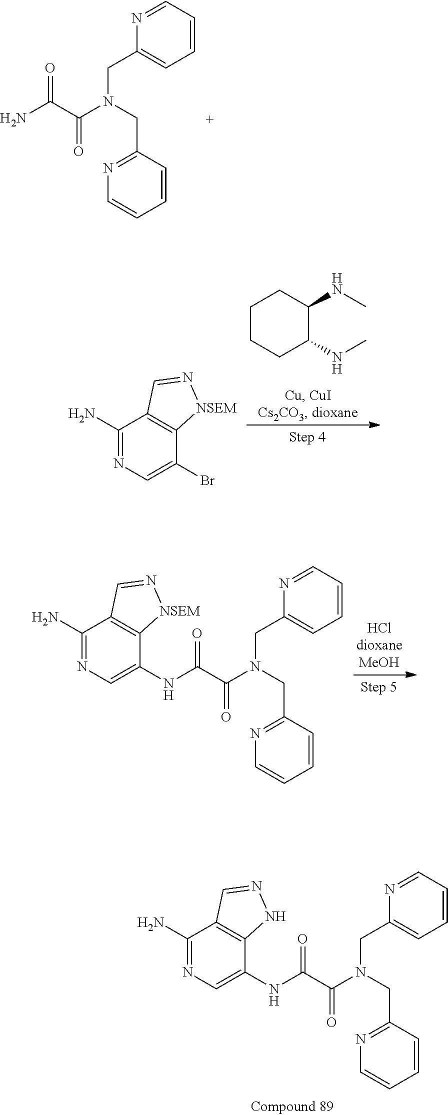

### Step 1:1-(2-pyridyl)-N-(2-pyridylmethyl)methanamine

2-pyridylmethanamine (1 g, 9.25 mmol, 953.29 μL), Sodium sulfate, anhydrous (19.70 g, 138.71 mmol, 7.35 mL) and pyridine-2-carbaldehyde (990.47 mg, 9.25 mmol, 881.20 μL) were mixed in DCM (15 mL) at 20° C. The resulting mixture was stirred at 20° C. for 12 hr, then filtered and concentrated in vacuo. The residue was dissolved in methanol (15 mL), cooled to 5° C. and Sodium Borohydride (384.80 mg, 10.17 mmol, 358.29 μL) was added. The reaction mixture was allowed to warm to 20° C. and stirred for 2 hr, then concentrated in vacuo. The residue was diluted with water (25 mL) and extracted with DCM (2×40 mL). The organic layer was separated, dried over anhydrous sodium sulfate and concentrated in vacuo to afford 1-(2-pyridyl)-N-(2-pyridylmethyl)methanamine (1.3 g, crude) as a brown oil.

LCMS (ESI): [M+H]⁺m/z: calcd 200.12; found 200.2; Rt=0.389 min.

### Step 2: ethyl 2-[bis(2-pyridylmethyl)amino]-2-oxo-acetate

1-(2-pyridyl)-N-(2-pyridylmethyl)methanamine (1.3 g, 4.57 mmol) and TEA (924.29 mg, 9.13 mmol, 1.27 mL) were dissolved in acetonitrile (20 mL), cooled with ice-water bath and then ethyl 2-chloro-2-oxoacetate (872.99 mg, 6.39 mmol, 714.39 μL) was added. Solution was stirred overnight at 20° C. The RM was concentrated in vacuo, redissolved in DCM (40 mL) and washed with water twice. Organic phase was dried over anhydrous sodium sulfate and concentrated in vacuo to give ethyl 2-[bis(2-pyridylmethyl)amino]-2-oxo-acetate (1.38 g, crude) as a brown oil.

LCMS (ESI): [M+H]⁺m/z: calcd 300.14; found 300.0; Rt=0.823 min.

### Step 3: N′,N′-bis(2-pyridylmethyl)oxamide

ethyl 2-[bis(2-pyridylmethyl)amino]-2-oxo-acetate (1.38 g, 3.46 mmol) was dissolved in MeOH (20 mL) saturated with NH3 (3.46 mmol) and stirred overnight. The RM was concentrated, redissolved in DCM, washed with water, dried over anhydrous sodium sulfate and concentrated in vacuo to give N′,N′-bis(2-pyridylmethyl)oxamide (0.63 g, crude) as a brown solid.

LCMS (ESI): [M+H]⁺m/z: calcd 271.12; found 271.2; Rt=0.503 min.

### Step 4: N-[4-amino-1-(2-trimethylsilylethoxymethyl)pyrazolo[4,3-c]pyridin-7-yl]-N′,N′-bis(2-pyridylmethyl)oxamide

Copper (1.65 mg, 25.90 μmol), Copper (I) iodide (49.32 mg, 258.98 μmol, 8.78 μL), cesium carbonate (337.53 mg, 1.04 mmol) were added to a stirred solution of N′,N′-bis(2-pyridylmethyl)oxamide (200 mg, 517.97 μmol), 7-bromo-1-(2-trimethylsilylethoxymethyl)pyrazolo[4,3-c]pyridin-4-amine (177.82 mg, 517.97 μmol) and rac-(1R,2R)—N1,N2-dimethylcyclohexane-1,2-diamine (143.67 mg, 1.01 mmol) in 1,4-dioxane (5 mL) under Ar atmosphere and stirred at 100° C. for 12 hr in closed vial. The reaction mixture was treated with water, then oily residue on the filter was dissolved in DCM (30 mL), dried over anhydrous sodium sulfate and evaporated in vacuo to give N-[4-amino-1-(2-trimethylsilylethoxymethyl)pyrazolo[4,3-c]pyridin-7-yl]-N′,N′-bis(2-pyridylmethyl)oxamide (150 mg, crude) as a brown solid.

LCMS (ESI): [M+H]⁺m/z: calcd 533.25; found 533.4; Rt=1.069 min.

### Step 5: The synthesis of N-(4-amino-1H-pyrazolo[4,3-c]pyridin-7-yl)-N′,N′-bis(2-pyridylmethyl)oxamide

N-[4-amino-1-(2-trimethylsilylethoxymethyl)pyrazolo[4,3-c]pyridin-7-yl]-N′,N′-bis(2-pyridylmethyl)oxamide (150 mg, 92.93 μmol) was dissolved in Dioxane (4 mL) saturated with HCl (10% by weight). MeOH (4 mL) was added for better solubility. The RM was stirred for 12 hr at 20° C., then concentrated in vacuo. The residue was dissolved in 3 mL of MeOH and subjected to HPLC. HPLC data: Device (Mobile Phase, Column): 2-10 min 45-60% water-methanol+NH3 to afford N-(4-amino-1H-pyrazolo[4,3-c]pyridin-7-yl)-N′,N′-bis(2-pyridylmethyl)oxamide (10.3 mg, 25.60 μmol, 27.54% yield) as a yellow solid.

¹H NMR (600 MHz, dmso) δ 4.25-5.12 (m, 4H), 6.57-6.94 (m, 2H), 7.24-7.31 (m, 2H), 7.31-7.36 (m, 1H), 7.38-7.44 (m, 1H), 7.57-7.66 (m, 1H), 7.73-7.82 (m, 2H), 8.11-8.18 (m, 1H), 8.47-8.55 (m, 2H), 9.79-10.58 (m, 1H), 12.70-13.36 (m, 1H).

LCMS (ESI): [M+H]⁺m/z: calcd 403.14; found 404.2; Rt=1.621 min.

### Example 87. Synthesis of N-(4-amino-1H-pyrazolo[4,3-c]pyridin-7-yl)-N′-benzyl-N′-[(6-methyl-2-pyridyl)methyl]oxamide (Compound 191)

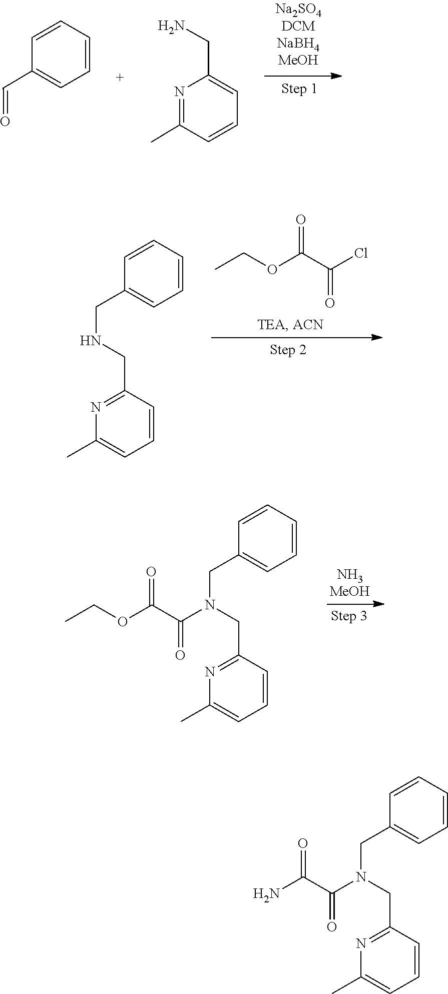

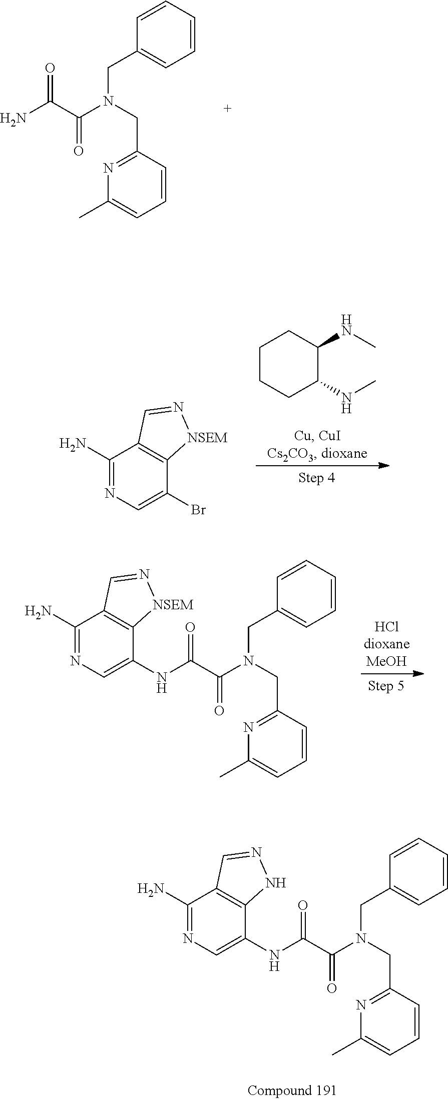

### Step 1: N-[(6-methyl-2-pyridyl)methyl]-1-phenyl-methanamine

(6-methyl-2-pyridyl)methanamine (1.00 g, 8.19 mmol), Sodium sulfate, anhydrous (17.44 g, 122.78 mmol, 6.51 mL) and benzaldehyde (868.66 mg, 8.19 mmol) were mixed in DCM (20 mL) at 20° C. The resulting mixture was stirred at 20° C. for 15 hr then filtered and concentrated in vacuo. The residue was dissolved in methanol (20 mL), cooled to 5° C. and Sodium Borohydride (340.62 mg, 9.00 mmol, 317.15 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford N-[(6-methyl-2-pyridyl)methyl]-1-phenyl-methanamine (1.26 g, crude) as a yellow oil.

LCMS (ESI): [M+H]⁺m/z: calcd 213.14; found 213.2; Rt=0.714 min.

### Step 2: ethyl 2-[benzyl-[(6-methyl-2-pyridyl)methyl]amino]-2-oxo-acetate

N-[(6-methyl-2-pyridyl)methyl]-1-phenyl-methanamine (1.26 g, 1.19 mmol) and TEA (240.24 mg, 2.37 mmol, 330.90 μL) were dissolved in acetonitrile (21.78 mL), cooled with ice-water bath and then ethyl 2-chloro-2-oxoacetate (324.15 mg, 2.37 mmol, 265.26 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-[benzyl-[(6-methyl-2-pyridyl)methyl]amino]-2-oxo-acetate (1.77 g, crude) as a red oil.

LCMS (ESI): [M+H]⁺m/z: calcd 313.16; found 313.0; Rt=1.049 min.

### Step 3: N′-benzyl-N′-[(6-methyl-2-pyridyl)methyl]oxamide

Ethyl 2-[benzyl-[(6-methyl-2-pyridyl)methyl]amino]-2-oxo-acetate (1.77 g, 1.70 mmol) was dissolved in MeOH (20 mL) saturated with NH3 (1.70 mmol) and stirred overnight. The RM was concentrated, redissolved in DCM, washed with water, dried over anhydrous sodium sulfate, and concentrated in vacuo. The residue was purified with FCC (gradient chloroform-acetonitrile) to give N′-benzyl-N′-[(6-methyl-2-pyridyl)methyl]oxamide (260 mg, crude) as a brown oil.

LCMS (ESI): [M+H]⁺m/z: calcd 284.14; found 284.2; Rt=0.836 min.

### Step 4: N-[4-amino-1-(2-trimethylsilylethoxymethyl)pyrazolo[4,3-c]pyridin-7-yl]-N′-benzyl-N′-[(6-methyl-2-pyridyl)methyl]oxamide

Copper (2.04 mg, 32.12 μmol), Copper (I) iodide (61.17 mg, 321.19 μmol, 10.88 μL), cesium carbonate (418.60 mg, 1.28 mmol) were added to a stirred solution of N′-benzyl-N′-[(6-methyl-2-pyridyl)methyl]oxamide (260 mg, 642.37 μmol), 7-bromo-1-(2-trimethylsilylethoxymethyl)pyrazolo[4,3-c]pyridin-4-amine (220.52 mg, 642.37 μmol) and rac-(1R,2R)—N1,N2-dimethylcyclohexane-1,2-diamine (178.17 mg, 1.25 mmol) in 1,4-dioxane (5 mL) under Ar atmosphere and stirred at 100° C. for 12 hr in closed vial. The reaction mixture was treated with water, then oily residue on the filter was dissolved in DCM (30 mL), dried over anhydrous sodium sulfate and evaporated in vacuo to give N-[4-amino-1-(2-trimethylsilylethoxymethyl)pyrazolo[4,3-c]pyridin-7-yl]-N′-benzyl-N′-[(6-methyl-2-pyridyl)methyl]oxamide (160 mg, crude) as a brown solid.

LCMS (ESI): [M+H]⁺m/z: calcd 546.27; found 546.0; Rt=1.128 min.

### Step 5: The synthesis of N-(4-amino-1H-pyrazolo[4,3-c]pyridin-7-yl)-N′-benzyl-N′-[(6-methyl-2-pyridyl)methyl]oxamide

N-[4-amino-1-(2-trimethylsilylethoxymethyl)pyrazolo[4,3-c]pyridin-7-yl]-N′-benzyl-N′-[(6-methyl-2-pyridyl)methyl]oxamide (93.91 mg, 92.93 μmol) was dissolved in Dioxane (4 mL) saturated with HCl (10% by weight). MeOH (4 mL) was added for better solubility. The RM was stirred for 12 hr at 20° C. then concentrated in vacuo. The residue was dissolved in 3 ml of MeOH and subjected to HPLC (Device (Mobile Phase, Column): 0.5-6.5 min 10-60% water-MeOH+FA 30 mL) to afford N-(4-amino-1H-pyrazolo[4,3-c]pyridin-7-yl)-N′-benzyl-N′-[(6-methyl-2-pyridyl)methyl]oxamide (18.9 mg, 40.96 μmol, 44.07% yield, HCOOH) as a yellow solid.

¹H NMR (600 MHz, dmso) δ 2.40-2.44 (m, 3H), 4.33-4.88 (m, 4H), 6.62-6.97 (m, 2H), 7.08-7.19 (m, 2H), 7.26-7.41 (m, 5H), 7.56-7.69 (m, 2H), 8.17 (d, 1H), 9.74-10.72 (m, 1H), 12.57-13.45 (m, 1H).

LCMS (ESI): [M+H]⁺m/z: calcd 416.2; found 416.2; Rt=1.715 min.

### Example 88. Synthesis of N-(4-amino-1H-pyrazolo[4,3-c]pyridin-7-yl)-N′-benzyl-N′-(cyclohexylmethyl)oxamide (Compound 175)

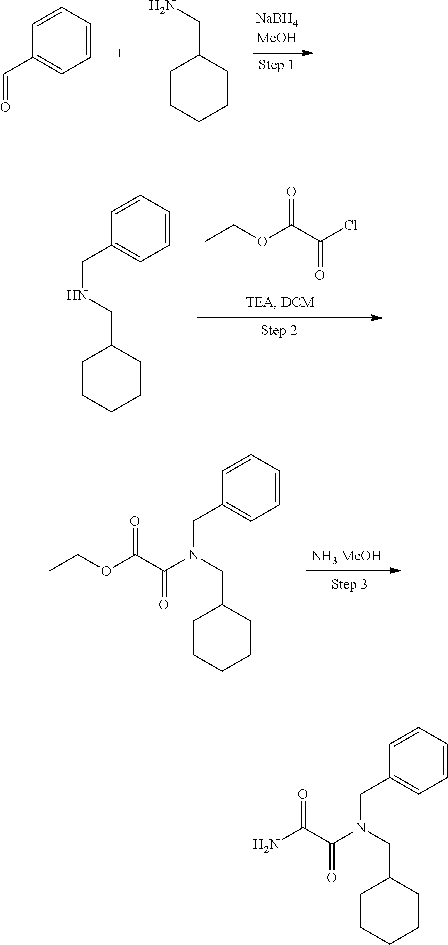

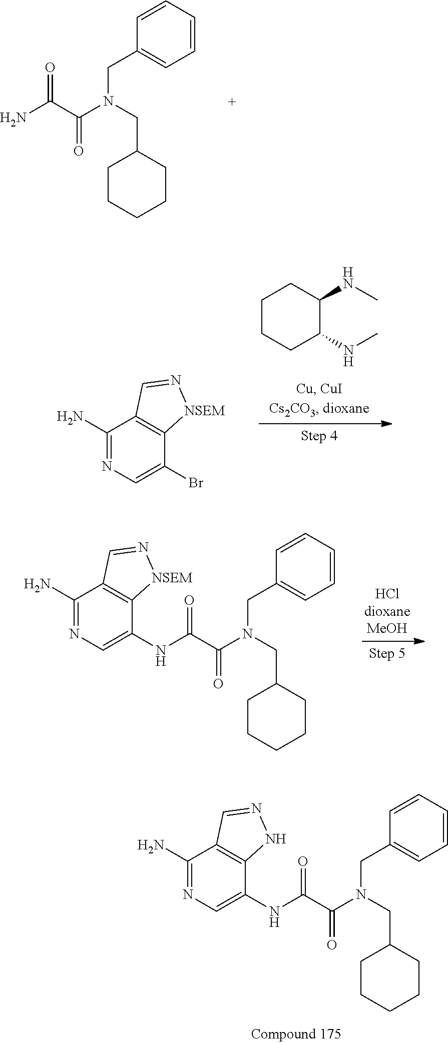

### Step 1: N-(cyclohexylmethyl)-1-phenyl-methanamine

Cyclohexylmethanamine (982.87 mg, 8.68 mmol, 1.13 mL)cyclohexylmethanamine (982.87 mg, 8.68 mmol, 1.13 mL) was added to a stirred solution of benzaldehyde (921.41 mg, 8.68 mmol) in Methanol (25 mL) stirred at 20° C. for 10 hr. Sodium Borohydride (328.46 mg, 8.68 mmol, 305.83 μL) was added, the reaction mixture was stirred for 2 hr additional. The reaction mixture was evaporated in vacuo. The residue was dissolved in DCM (50 mL) and washed with water (2×15 mL). The organic layer was dried over anhydrous sodium sulfate and filtered. DCM was evaporated under reduce pressure to give N-(cyclohexylmethyl)-1-phenyl-methanamine (1.6 g, 7.87 mmol, 90.63% yield) as a yellow oil.

LCMS (ESI): [M+H]⁺m/z: calcd 204.18; found 204.2; Rt=0.809 min.

### Step 2: ethyl 2-[benzyl(cyclohexylmethyl)amino]-2-oxo-acetate

N-(cyclohexylmethyl)-1-phenyl-methanamine (1.6 g, 7.87 mmol) and TEA (796.29 mg, 7.87 mmol, 1.10 mL) were dissolved in DCM (50.53 mL), cooled with ice-water bath and then ethyl 2-chloro-2-oxo-acetate (1.07 g, 7.87 mmol, 879.23 μL) in 20 mL of DCM was added dropwise in 10 min. The solution was stirred overnight. The reaction mixture was washed with an aqueous solution of NaHCO₃, dried over anhydrous sodium sulfate and evaporated to give ethyl 2-[benzyl(cyclohexylmethyl)amino]-2-oxo-acetate (1.91 g, 6.30 mmol, 80.00% yield) as a yellow oil.

LCMS (ESI): [M+H]⁺m/z: calcd 204.18; found 204.2; Rt=0.957 min.

### Step 3: N′-benzyl-N′-(cyclohexylmethyl)oxamide

Ethyl 2-[benzyl(cyclohexylmethyl)amino]-2-oxo-acetate (1.89 g, 6.22 mmol) was dissolved in NH3/methanol (30 mL) solution (10% by weight) and stirred overnight, then concentrated in vacuo to give N′-benzyl-N′-(cyclohexylmethyl)oxamide (1.5 g, 5.47 mmol, 87.87% yield) as a yellow oil.

LCMS (ESI): [M+H]⁺m/z: calcd 275.18; found 275.2; Rt=1.262 min.

### Step 4: N-[4-amino-1-(2-trimethylsilylethoxymethyl)pyrazolo[4,3-c]pyridin-7-yl]-N′-benzyl-N′-(cyclohexylmethyl)oxamide

7-bromo-1-(2-trimethylsilylethoxymethyl)pyrazolo[4,3-c]pyridin-4-amine (0.2 g, 582.59 μmol), N′-benzyl-N′-(cyclohexylmethyl)oxamide (201.51 mg, 734.48 μmol), Copper (I) iodide (41.96 mg, 220.34 μmol, 7.47 μL), Cesium carbonate (478.62 mg, 1.47 mmol) and (1R,2R)—N1,N2-dimethylcyclohexane-1,2-diamine (47.01 mg, 330.52 μmol) were mixed in Dioxane under argon, and then stirred overnight at 95° C. for 12 hr in vial. The reaction mixture was filtered, then evaporated. The residue was dissolved in 15 mL of DCM washed with an aqueous ammonium solution, dried over anhydrous sodium sulfate and evaporated to give crude N-[4-amino-1-(2-trimethylsilylethoxymethyl)pyrazolo[4,3-c]pyridin-7-yl]-N′-benzyl-N′-(cyclohexylmethyl)oxamide (0.35 g, 652.09 μmol, 88.78% yield)

LCMS (ESI): [M+H]⁺m/z: calcd 537.3; found 537.4; Rt=1.175 min.

### Step 5: The synthesis of N-(4-amino-1H-pyrazolo[4,3-c]pyridin-7-yl)-N′-benzyl-N′-(cyclohexylmethyl)oxamide

N-[4-amino-1-(2-trimethylsilylethoxymethyl)pyrazolo[4,3-c]pyridin-7-yl]-N′-benzyl-N′-(cyclohexylmethyl)oxamide (351.35 mg, 654.59 μmol) was dissolved in MeOH (1 mL) and dioxane/HCl (13.09 mmol, 1 mL) was added thereto. Then it was stirred at rt for 2 hr. The reaction mixture was evaporated. The crude product was purified by HPLC (2-10 min 30-50% MeOH+NH3, 30 mL/min ((loading pump 4 mL MeOH+NH3) column: XBridge BEH C18 100×20 5 microM) to afford N-(4-amino-1H-pyrazolo[4,3-c]pyridin-7-yl)-N′-benzyl-N′-(cyclohexylmethyl)oxamide (0.054 g, 132.85 μmol, 20.29% yield) as a yellow solid. ¹H NMR (600 MHz, dmso) 8 0.37-0.90 (m, 2H), 0.97-1.20 (m, 3H), 1.40-1.72 (m, 6H), 2.92-3.28 (m, 2H), 4.19-4.77 (m, 2H), 6.36-7.11 (m, 2H), 7.23-7.32 (m, 2H), 7.32-7.39 (m, 3H), 7.47-7.74 (m, 1H), 8.11-8.25 (m, 1H), 9.50-10.53 (m, 1H), 12.65-13.34 (m, 1H).

LCMS (ESI): [M+H]⁺m/z: calcd 407.25; found 407.4; Rt=3.133 min.

### Example 89. Synthesis of N-(4-amino-1H-pyrazolo[4,3-c]pyridin-7-yl)-N′-benzyl-N′-(tetrahydropyran-2-ylmethyl)oxamide (Compound 148)

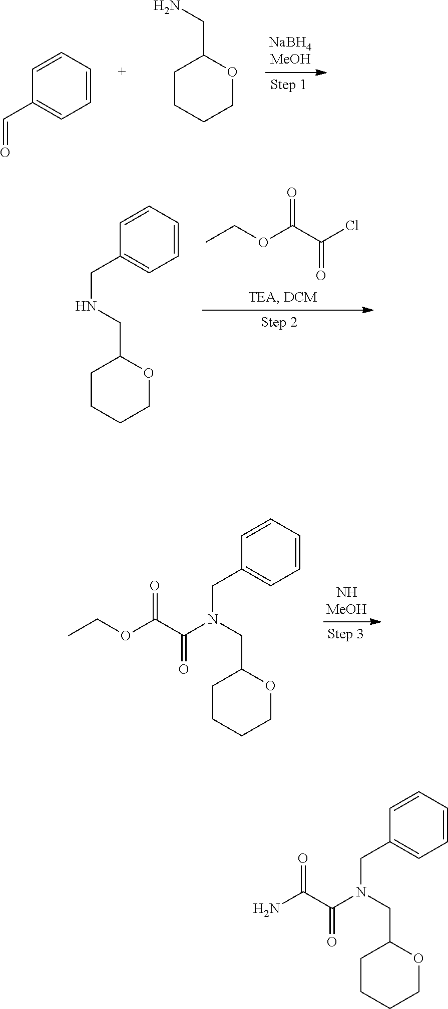

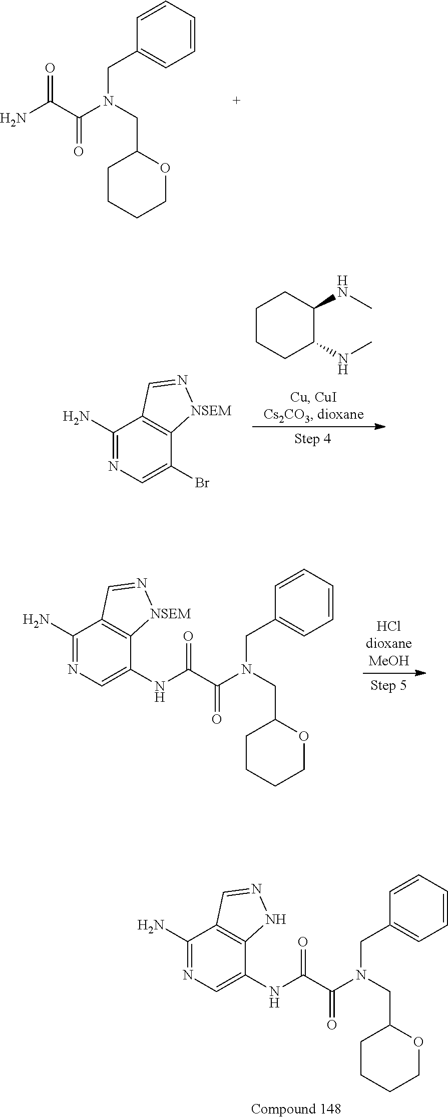

### Step 1:1-phenyl-N-(tetrahydropyran-2-ylmethyl)methanamine

Tetrahydropyran-2-ylmethanamine (1 g, 8.68 mmol) tetrahydropyran-2-ylmethanamine (1 g, 8.68 mmol) was added to a stirred solution of benzaldehyde (921.41 mg, 8.68 mmol) in Methanol (25 mL) stirred at 20° C. for 10 hr. Sodium Borohydride (328.46 mg, 8.68 mmol, 305.83 μL) was added, the reaction mixture was stirred for 2 hr additional. The reaction mixture was evaporated in vacuo. The residue was dissolved in DCM (50 mL) and washed with water (2×15 mL). The organic layer was dried over anhydrous sodium sulfate and filtered. DCM was evaporated under reduce pressure to give 1-phenyl-N-(tetrahydropyran-2-ylmethyl)methanamine (1.6 g, 7.79 mmol, 89.76% yield) as a yellow oil.

LCMS (ESI): [M+H]⁺m/z: calcd 206.16; found 206.2; Rt=0.697 min.

### Step 2: ethyl 2-[benzyl(tetrahydropyran-2-ylmethyl)amino]-2-oxo-acetate

1-phenyl-N-(tetrahydropyran-2-ylmethyl)methanamine (2.04 g, 9.94 mmol) and TEA (1.01 g, 9.94 mmol, 1.39 mL) were dissolved in DCM (50 mL), cooled with ice-water bath and then ethyl 2-chloro-2-oxo-acetate (1.36 g, 9.94 mmol, 1.11 mL) in 20 mL of DCM was added dropwise in 10 min. The solution was stirred overnight. The reaction mixture was washed with an aqueous solution of NaHCO₃, dried over anhydrous sodium sulfate and evaporated to give ethyl 2-[benzyl(tetrahydropyran-2-ylmethyl)amino]-2-oxo-acetate (1.9 g, 6.22 mmol, 62.61% yield) as a yellow oil.

LCMS (ESI): [M−H]⁻ m/z: calcd 537.26; found 537.0; Rt=1.121 min.

### Step 3: N′-benzyl-N′-(tetrahydropyran-2-ylmethyl)oxamide

Ethyl 2-[benzyl(tetrahydropyran-2-ylmethyl)amino]-2-oxo-acetate (1.9 g, 6.22 mmol) was dissolved in NH3/methanol (30 mL) solution (10% by weight) and stirred overnight, then concentrated in vacuo to give N′-benzyl-N′-(tetrahydropyran-2-ylmethyl)oxamide (1.1 g, 3.98 mmol, 63.98% yield) as a yellow oil.

LCMS (ESI): [M+H]⁺m/z: calcd 277.16; found 277.2; Rt=1.090 min.

### Step 4: N-[4-amino-1-(2-trimethylsilylethoxymethyl)pyrazolo[4,3-c]pyridin-7-yl]-N′-benzyl-N′-(tetrahydropyran-2-ylmethyl)oxamide

7-bromo-1-(2-trimethylsilylethoxymethyl)pyrazolo[4,3-c]pyridin-4-amine (252.14 mg, 734.48 μmol), N′-benzyl-N′-(tetrahydropyran-2-ylmethyl)oxamide (202.96 mg, 734.48 μmol), Copper (I) iodide (41.96 mg, 220.34 μmol, 7.47 μL), Cesium carbonate (478.62 mg, 1.47 mmol) and (1R,2R)—N1,N2-dimethylcyclohexane-1,2-diamine (47.01 mg, 330.52 μmol) were mixed in Dioxane under argon, and then stirred overnight at 95° C. for 12 hr in vial. The reaction mixture was filtered, then evaporated. The residue was dissolved in 15 mL of DCM, washed with an aqueous ammonium solution, dried over anhydrous sodium sulfate and evaporated to afford N-[4-amino-1-(2-trimethylsilylethoxymethyl)pyrazolo[4,3-c]pyridin-7-yl]-N′-benzyl-N′-(tetrahydropyran-2-ylmethyl)oxamide (0.37 g, 686.82 μmol, 93.51% yield).

LCMS (ESI): [M+H]⁺m/z: calcd 539.28; found 539.2; Rt=1.069 min.

### Step 5: The synthesis of N-(4-amino-1H-pyrazolo[4,3-c]pyridin-7-yl)-N′-benzyl-N′-(tetrahydropyran-2-ylmethyl)oxamide (Compound 148)

N-[4-amino-1-(2-trimethylsilylethoxymethyl)pyrazolo[4,3-c]pyridin-7-yl]-N′-benzyl-N′-(tetrahydropyran-2-ylmethyl)oxamide (352.64 mg, 654.59 μmol) was dissolved in MeOH (1 mL) and dioxane/HCl (13.09 mmol, 1 mL) was added thereto. Then it was stirred at rt for 2 hr. The reaction mixture was evaporated. The crude product was purified by HPLC (2-10 min 30-50% MeOH+NH3, 30 mL/min ((loading pump 4 mL MeOH+NH3) column: XBridge BEH C18 100×20, 5 microM) to afford N-(4-amino-1H-pyrazolo[4,3-c]pyridin-7-yl)-N′-benzyl-N′-(tetrahydropyran-2-ylmethyl)oxamide (74.5 mg, 182 μmol, 27.9% yield) as a yellow solid.

¹H NMR (600 MHz, dmso) 8 0.33-1.35 (m, 2H), 1.34-1.70 (m, 4H), 1.70-2.08 (m, 1H), 2.81-3.17 (m, 1H), 3.48-3.61 (m, 2H), 3.72-3.94 (m, 1H), 4.23-5.02 (m, 2H), 6.41-6.91 (m, 2H), 7.08-7.41 (m, 5H), 7.49-7.72 (m, 1H), 8.08-8.26 (m, 1H), 9.49-10.54 (m, 1H), 12.46-13.51 (m, 1H).

LCMS (ESI): [M+H]⁺m/z: calcd 408.22; found 409.4; Rt=2.711 min.

### Example 90. Synthesis of N-(4-amino-1H-pyrazolo[4,3-c]pyridin-7-yl)-N′,N′-bis [1-(4-fluorophenyl)ethyl]oxamide (Compound 112)

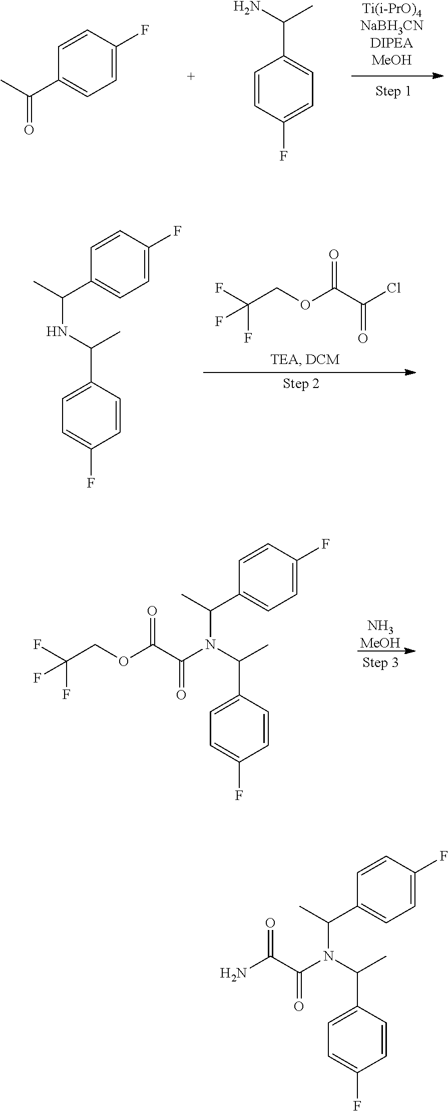

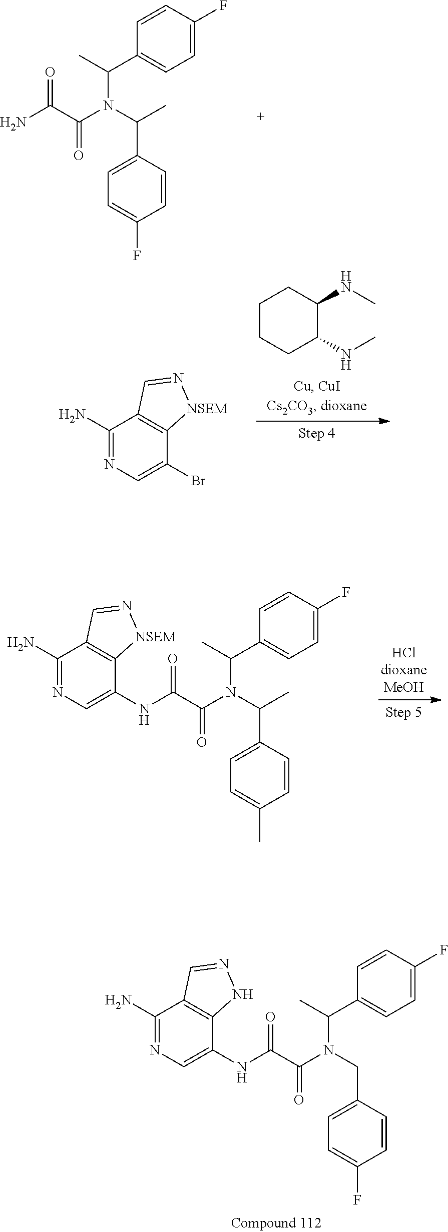

### Step 1:1-(4-fluorophenyl)-N-[1-(4-fluorophenyl)ethyl]ethanamine

To a mixture of 1-(4-fluorophenyl) ethanone (1 g, 7.24 mmol, 876.42 μL) and 1-(4-fluorophenyl)ethanamine (1.01 g, 7.24 mmol, 978.12 μL) in MeOH (50 mL) DIPEA (1.87 g, 14.48 mmol, 2.52 mL) and Titanium (IV) isopropoxide, 95% (4.11 g, 14.48 mmol, 4.31 mL) was added and stirred at 25° C. for 14 hr. Sodium cyanoborohydride (909.84 mg, 14.48 mmol) was added and reaction mixture was stirred at 25° C. overnight. Water (10 mL) was added to reaction mixture and was evaporated in vacuo. The residue was dissolved in EtOAc (50 mL), washed with water (50 mL), dried over anhydrous sodium sulfate and evaporated in vacuo to afford crude product (475 mg), which was purified by CC (Hexane-EtOAc+TEA as an eluent mixture) to afford 1-(4-fluorophenyl)-N-[1-(4-fluorophenyl)ethyl]ethanamine (0.2 g, 765.38 μmol, 10.57% yield) as a light-yellow solid.

LCMS (ESI): [M+H]⁺m/z: calcd 262.14; found 262.2; Rt=0.654 min.

### Step 2:2,2,2-trifluoroethyl 2-[bis [1-(4-fluorophenyl)ethyl]amino]-2-oxo-acetate

1-(4-fluorophenyl)-N-[1-(4-fluorophenyl)ethyl]ethanamine (0.2 g, 765.38 μmol) was dissolved in DCM (19.68 mL) and TEA (232.35 mg, 2.30 mmol, 320.04 μL) was added. The reaction mixture was cooled and 2,2,2-trifluoroethyl 2-chloro-2-oxo-acetate (174.97 mg, 918.45 μmol) was added dropwise. After that it was stirred at rt overnight. Then water (10 mL) was added, organic layer was stirred with brine and dried over anhydrous sodium sulfate, evaporated in vacuum to afford 2,2,2-trifluoroethyl 2-[bis [1-(4-fluorophenyl)ethyl]amino]-2-oxo-acetate (0.3 g, 722.28 μmol, 94.37% yield) as a yellow gum.

LCMS (ESI): [M+Na]⁺m/z: calcd 437.12; found 438.0; Rt=1.650 min.

### Step 3: N′,N′-bis [1-(4-fluorophenyl)ethyl]oxamide

2,2,2-trifluoroethyl 2-[bis [1-(4-fluorophenyl)ethyl]amino]-2-oxo-acetate (0.3 g, 722.28 μmol) was dissolved in MeOH/NH3 (10 mL) and stirred overnight at rt. Then it was evaporated in vacuum to afford N′,N′-bis [1-(4-fluorophenyl)ethyl]oxamide (0.2 g, 601.79 μmol, 83.32% yield) as a light-yellow solid.

LCMS (ESI): [M+Na]⁺m/z: calcd 355.14; found 355.0; Rt=1.014 min.

### Step 4: N-[4-amino-1-(2-trimethylsilylethoxymethyl)pyrazolo[4,3-c]pyridin-7-yl]-N′,N′-bis [1-(4-fluorophenyl)ethyl]oxamide

A mixture of 7-bromo-1-(2-trimethylsilylethoxymethyl)pyrazolo[4,3-c]pyridin-4-amine (113.62 mg, 330.98 μmol), N′,N′-bis [1-(4-fluorophenyl)ethyl]oxamide (0.1 g, 300.89 μmol), Cesium carbonate (147.06 mg, 451.34 μmol), rac-(1R,2R)—N1,N2-dimethylcyclohexane-1,2-diamine (34.24 mg, 240.71 μmol) and CuI (34.38 mg, 180.54 μmol, 6.12 μL) with a few mg of Cu(956.03 μg, 15.04 μmol) in dioxane (3.99 mL) was stirred in a sealed vial under argon at 110° C. for 48 hr. The resulting mixture was cooled down and filtered through silica gel to afford N-[4-amino-1-(2-trimethylsilylethoxymethyl)pyrazolo[4,3-c]pyridin-7-yl]-N′,N′-bis [1-(4-fluorophenyl)ethyl]oxamide (0.17 g, 285.85 μmol, 95.00% yield) as a brown gum.

LCMS (ESI): [M+H]⁺m/z: calcd 595.27; found 595.2; Rt=1.404 min.

### Step 5: The synthesis of N-(4-amino-1H-pyrazolo[4,3-c]pyridin-7-yl)-N′,N′-bis [1-(4-fluorophenyl)ethyl]oxamide (Compound 112)

N-[4-amino-1-(2-trimethylsilylethoxymethyl)pyrazolo[4,3-c]pyridin-7-yl]-N′,N′-bis [1-(4-fluorophenyl)ethyl]oxamide (0.17 g, 285.85 μmol) was dissolved in MeOH (5 mL) and diox/HCl (285.85 μmol, 1 mL) was added. Then mixture was stirred at rt 12 hr. The solution was evaporated and submitted to reverse phase HPLC (column: SunFire C18 100×19 mm, 5 μm; mobile phase: 15-40% 0.5-6.5 min water-MeCN+FA, flow rate: 30 mL/min) to afford N-(4-amino-1H-pyrazolo[4,3-c]pyridin-7-yl)-N′,N′-bis [1-(4-fluorophenyl)ethyl]oxamide (15.7 mg, 30.75 μmol, 10.76% yield, HCOOH) as a brown solid.

LCMS (ESI): [M+H]⁺m/z: calcd 465.21; found 465.2; Rt=1.494 min.

### Example 91. Synthesis of N-(4-amino-1H-pyrazolo[4,3-c]pyridin-7-yl)-N′-benzyl-N′-[[5-(trifluoromethyl)-2-pyridyl]methyl]oxamide (Compound 108)

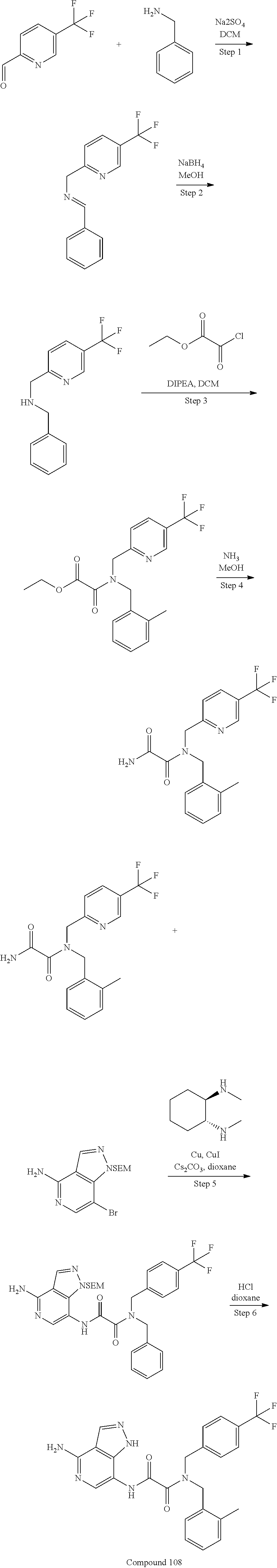

### Step 1: (E)-N-benzyl-1-[5-(trifluoromethyl)-2-pyridyl]methanimine

5-(trifluoromethyl)pyridine-2-carbaldehyde (3.5 g, 19.99 mmol), phenylmethanamine (2.14 g, 19.99 mmol) and Sodium sulfate, anhydrous (5.00 g, 35.22 mmol, 1.87 mL) was mixed in DCM (50 mL) and stirred at RT overnight. Upon completion, the reaction mixture was filtered and filtrate was concentrated under reduced pressure to afford (E)-N-benzyl-1-[5-(trifluoromethyl)-2-pyridyl]methanimine (4.5 g, crude) which was directly used in the next step without purification and analytical data collection.

### Step 2:1-phenyl-N-[[5-(trifluoromethyl)-2-pyridyl]methyl]methanamine

Sodium Borohydride (966.41 mg, 25.54 mmol, 899.83 μL) was added portionwise to a stirred solution of (E)-N-benzyl-1-[5-(trifluoromethyl)-2-pyridyl]methanimine (4.5 g, 17.03 mmol) in methanol (60 mL). The reaction mixture was stirred overnight and concentrated under reduced pressure. The residue was taken up in DCM, washed with NaHCO₃ solution, dried over anhydrous sodium sulfate and concentrated under reduced pressure to afford 1-phenyl-N-[[5-(trifluoromethyl)-2-pyridyl]methyl]methanamine (3.5 g, crude).

LCMS (ESI): [M+H]⁺m/z: calcd 267.11; found 267.2; Rt=0.744 min.

### Step 3: ethyl 2-[benzyl-[[5-(trifluoromethyl)-2-pyridyl]methyl]amino]-2-oxo-acetate

Ethyl 2-chloro-2-oxo-acetate (1.97 g, 14.46 mmol, 1.62 mL) was added dropwise to an ice bath cooled stirred solution of 1-phenyl-N-[5-(trifluoromethyl)-2-pyridyl]methyl]methanamine (3.5 g, 13.14 mmol) and DIPEA (2.21 g, 17.09 mmol, 2.98 mL) in DCM (40 mL). The reaction mixture was stirred overnight. Upon completion, the reaction mixture was washed with water. The organic layer was separated, dried over anhydrous sodium sulfate and concentrated under reduced pressure to afford ethyl 2-[benzyl-[5-(trifluoromethyl)-2-pyridyl]methyl]amino]-2-oxo-acetate (2.8 g, 7.64 mmol, 58.15% yield) which was used in the next step without purification.

LCMS (ESI): [M+H]⁺m/z: calcd 367.13; found 367.0; Rt=1.498 min.

### Step 4: N′-benzyl-N′-[[5-(trifluoromethyl)-2-pyridyl]methyl]oxamide

Ethyl 2-[benzyl-[[5-(trifluoromethyl)-2-pyridyl]methyl]amino]-2-oxo-acetate (2.8 g, 7.64 mmol) was dissolved in saturated NH3/methanol solution and stirred overnight. Upon completion, the reaction mixture was concentrated under reduced pressure to afford N′-benzyl-N′-[5-(trifluoromethyl)-2-pyridyl]methyl]oxamide (2.5 g, crude).

LCMS (ESI): [M−H]⁻m/z: calcd 338.11; found 338.2; Rt=1.236 min.

### Step 5: N-[4-amino-1-(2-trimethylsilylethoxymethyl)pyrazolo[4,3-c]pyridin-7-yl]-N′-benzyl-N′-[5-(trifluoromethyl)-2-pyridyl]methyl]oxamide

N′-benzyl-N′-[[5-(trifluoromethyl)-2-pyridyl]methyl]oxamide (0.5 g, 1.48 mmol), 7-bromo-1-(2-trimethylsilylethoxymethyl)pyrazolo[4,3-c]pyridin-4-amine (508.89 mg, 1.48 mmol), copper (0.05 g, 786.78 μmol), Copper (I) iodide (282.32 mg, 1.48 mmol, 50.23 μL), (1S,2S)-N,N′-Bis -methyl-1,2-cyclohexane-diamine (210.85 mg, 1.48 mmol, 233.76 μL) were mixed in dioxane (10 mL). The reaction mixture was stirred under Ar atmosphere at 100° C. for 48 hr. Upon completion, the mixture was diluted with water and extracted with DCM. Organic layer was separated, dried over anhydrous sodium sulfate, and concentrated under reduced pressure. The residue was submitted to HPLC (2-10 min 30-85% MeOH+FA 30 mL/min ((loading pump 4 mL MeOH), column: Chromatorex C18 100×19 5 microM) to afford N-[4-amino-1-(2-trimethylsilylethoxymethyl)pyrazolo[4,3-c]pyridin-7-yl]-N′-benzyl-N′-[5-(trifluoromethyl)-2-pyridyl]methyl]oxamide (0.055 g, 91.72 μmol, 6.19% yield).

LCMS (ESI): [M+H]⁺m/z: calcd 600.28; found 600.3; Rt=1.281 min.

### Step 6: The synthesis of N-(4-amino-1H-pyrazolo[4,3-c]pyridin-7-yl)-N′-benzyl-N′-[[5-(trifluoromethyl)-2-pyridyl]methyl]oxamide

N-[4-amino-1-(2-trimethylsilylethoxymethyl)pyrazolo[4,3-c]pyridin-7-yl]-N′-benzyl-N′-[[5-(trifluoromethyl)-2-pyridyl]methyl]oxamide (0.055 g, 91.72 μmol) was dissolved in HCl/dioxane solution and stirred at 20° C. for 4 hr. Upon completion, reaction mixture was concentrated under reduced pressure and the residue was submitted to HPLC (2-10 min 0-85% ACN+FA, 30 mL/min ((loading pump 4 mL ACN), column: Cromatorex C18 100×19, 5 microM) to afford N-(4-amino-1H-pyrazolo[4,3-c]pyridin-7-yl)-N′-benzyl-N′-[[5-(trifluoromethyl)-2-pyridyl]methyl]oxamide (0.02 g, 42.61 μmol, 46.45% yield) as a beige solid.

¹H NMR (600 MHz, DMSO-d₆) δ 4.17-5.08 (m, 4H), 6.06-7.02 (m, 2H), 7.24-7.42 (m, 5H), 7.50-7.69 (m, 2H), 8.14 (d, 1H), 8.18 (d, 1H), 8.89 (d, 1H), 9.68-10.78 (m, 1H), 12.66-13.52 (m, 1H).

LCMS (ESI): [M+H]⁺m/z: calcd 470.17; found 470.2; Rt=2.757 min.

### Example 92. Synthesis of N-(4-amino-1H-pyrazolo[4,3-c]pyridin-7-yl)-N′-benzyl-N′-[(5-chloro-2-pyridyl)methyl]oxamide (Compound 158)

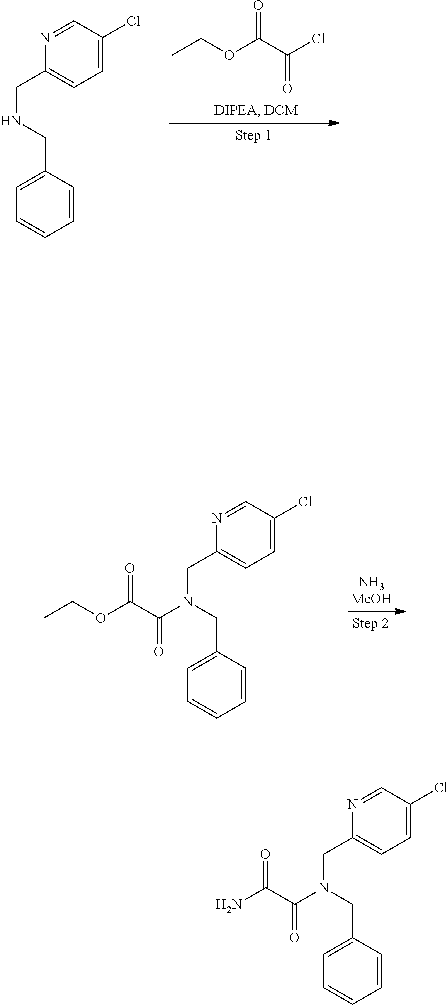

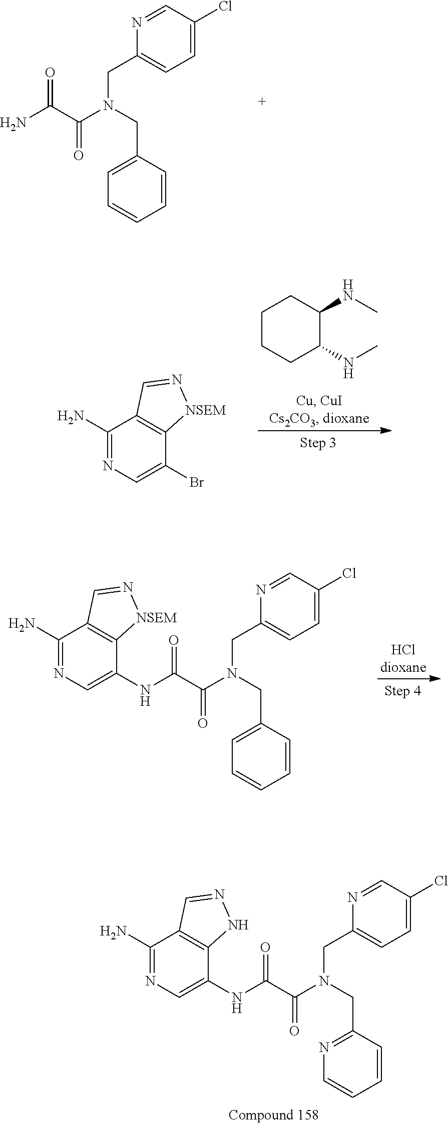

### Step 1: ethyl 2-[benzyl-[(5-chloro-2-pyridyl)methyl]amino]-2-oxo-acetate

Ethyl 2-chloro-2-oxo-acetate (3.94 g, 28.83 mmol, 3.22 mL) was added dropwise to an ice bath cooled stirred solution of N-[(5-chloro-2-pyridyl)methyl]-1-phenyl-methanamine (6.1 g, 26.21 mmol) and DIPEA (4.40 g, 34.08 mmol, 5.94 mL) in DCM (60 mL). The reaction mixture was stirred overnight. Upon completion, the reaction mixture was washed with water. The organic layer was separated, dried over anhydrous sodium sulfate, and concentrated under reduced pressure. The residue was purified by flash column chromatography (Interchim; 120 g SiO₂, HEX-MTBE from 0~100%, flow rate=70 mL/min, cv=7.2) to afford ethyl 2-[benzyl-[(5-chloro-2-pyridyl)methyl]amino]-2-oxo-acetate (4 g, 12.02 mmol, 45.85% yield).

LCMS (ESI): [M+H]⁺m/z: calcd 333.1; found 333.2; Rt=1.461 min.

### Step 2: N′-benzyl-N′-[(5-chloro-2-pyridyl)methyl]oxamide

Ethyl 2-[benzyl-[(5-chloro-2-pyridyl)methyl]amino]-2-oxo-acetate (2 g, 6.01 mmol) was dissolved in saturated NH₃/methanol solution and stirred overnight. Upon completion, the reaction mixture was concentrated under reduced pressure to afford N′-benzyl-N′-[(5-chloro-2-pyridyl)methyl]oxamide (1.8 g, 5.93 mmol, 98.60% yield) which was used in the next step without purification.

LCMS (ESI): [M+H]⁺m/z: calcd 304.09; found 304.2; Rt=1.147 min.

### Step 3: N-[4-amino-1-(2-trimethylsilylethoxymethyl)pyrazolo[4,3-c]pyridin-7-yl]-N′-benzyl-N′-[(5-chloro-2-pyridyl)methyl]oxamide

N′-benzyl-N′-[(5-chloro-2-pyridyl)methyl]oxamide (0.4 g, 1.32 mmol), 7-bromo-1-(2-trimethylsilylethoxymethyl)pyrazolo[4,3-c]pyridin-4-amine (452.09 mg, 1.32 mmol), copper (0.05 g, 786.78 μmol), Copper (I) iodide (250.80 mg, 1.32 mmol, 44.63 μL), (1S,2S)-N,N′-Bis -methyl-1,2-cyclohexane-diamine (187.32 mg, 1.32 mmol, 207.67 μL) were mixed in dioxane (12 mL). The reaction mixture was stirred under Ar atmosphere at 100° C. for 48 hr. Upon completion, the mixture was diluted with water and extracted with DCM. Organic layer was separated, dried over anhydrous sodium sulfate and concentrated under reduced pressure. The residue was submitted to HPLC (2-10 min 30-85% MeOH+FA 30 mL/min (loading pump 4 mL MeOH) column: Chromatorex C18 100×19 5 microM) to afford N-[4-amino-1-(2-trimethylsilylethoxymethyl)pyrazolo[4,3-c]pyridin-7-yl]-N′-benzyl-N′-[(5-chloro-2-pyridyl)methyl]oxamide (0.026 g, 45.93 μmol, 3.49% yield).

LCMS (ESI): [M+H]⁺m/z: calcd 566.25; found 566.2; Rt=1.259 min.

### Step 4: The synthesis of N-(4-amino-1H-pyrazolo[4,3-c]pyridin-7-yl)-N′-benzyl-N′-[(5-chloro-2-pyridyl)methyl]oxamide

N-[4-amino-1-(2-trimethylsilylethoxymethyl)pyrazolo[4,3-c]pyridin-7-yl]-N′-benzyl-N′-[(5-chloro-2-pyridyl)methyl]oxamide (0.026 g, 45.93 μmol) was dissolved in HCl/dioxane solution (2 mL) and stirred at 20° C. for 4 hr. Upon completion, the reaction mixture was concentrated under reduced pressure and the residue was purified by reverse phase chromatography (2-10 min 0-90% ACN+FA, 30 mL/min ((loading pump 4 mL ACN+FA) column: Cromatorex C18 100×19, 5 microM) to afford N-(4-amino-1H-pyrazolo[4,3-c]pyridin-7-yl)-N′-benzyl-N′-[(5-chloro-2-pyridyl)methyl]oxamide (0.009 g, 20.65 μmol, 44.96% yield) as a yellow solid.

¹H NMR (600 MHz, DMSO-d₆) δ 4.11-4.92 (m, 4H), 6.32-7.00 (m, 2H), 7.26-7.45 (m, 6H), 7.54-7.67 (m, 1H), 7.69-7.93 (m, 1H), 8.14-8.19 (m, 1H), 8.51-8.58 (m, 1H), 9.79-10.71 (m, 1H), 12.67-13.32 (m, 1H).

LCMS (ESI): [M+H]⁺m/z: calcd 436.14; found 436.2; Rt=2.583 min.

### Example 93. Synthesis of N-(4-amino-1H-pyrazolo[4,3-c]pyridin-7-yl)-N′-benzyl-N′-[(4-methyl-2-pyridyl)methyl]oxamide (Compound 156)

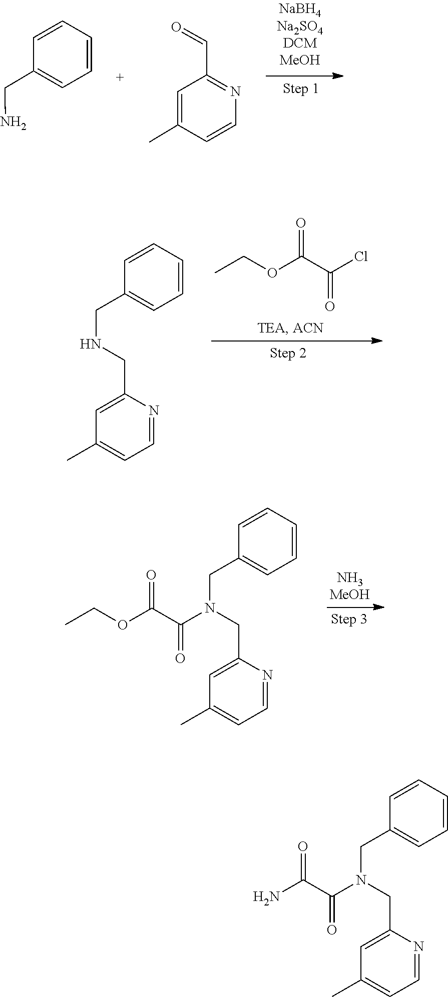

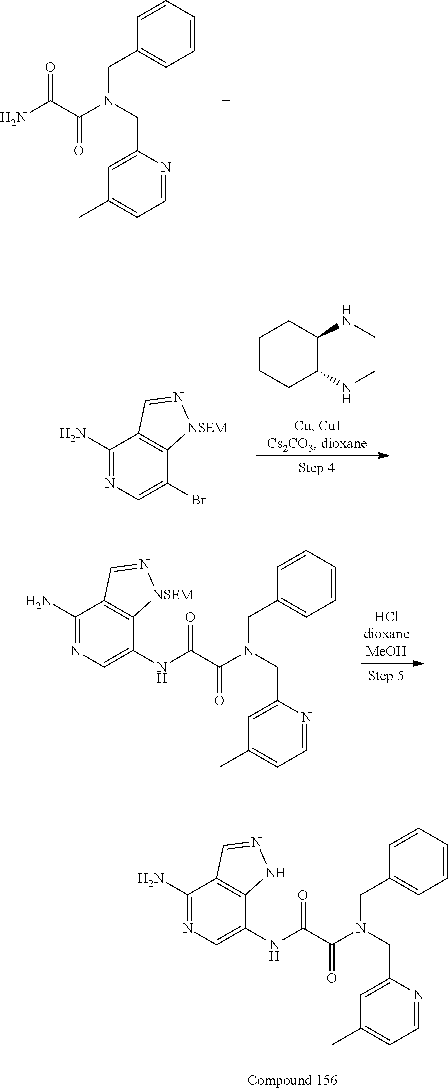

### Step 1: N-[(4-methyl-2-pyridyl)methyl]-1-phenyl-methanamine

4-methylpyridine-2-carbaldehyde (1 g, 8.26 mmol), Sodium sulfate, anhydrous (17.59 g, 123.83 mmol, 6.56 mL) and phenylmethanamine (884.56 mg, 8.26 mmol) were mixed in DCM (20 mL) at 20° C. and stirred for 12 hr, then filtered and concentrated in vacuo. The residue was dissolved in methanol (20 mL), cooled to 5° C. and Sodium Borohydride (343.52 mg, 9.08 mmol, 319.85 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford N-[(4-methyl-2-pyridyl)methyl]-1-phenyl-methanamine (1.45 g, crude) as a yellow liquid.

LCMS (ESI): [M+H]⁺m/z: calcd 213.14; found 213.2; Rt=0.781 min.

### Step 2: ethyl 2-[benzyl-[(4-methyl-2-pyridyl)methyl]amino]-2-oxo-acetate

N-[(4-methyl-2-pyridyl)methyl]-1-phenyl-methanamine (1.45 g, 6.15 mmol) and TEA (1.24 g, 12.29 mmol, 1.71 mL) were dissolved in acetonitrile (20 mL), cooled with ice-water bath and then ethyl 2-chloro-2-oxoacetate (1.01 g, 7.38 mmol, 824.20 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-[benzyl-[(4-methyl-2-pyridyl)methyl]amino]-2-oxo-acetate (1.8 g, 5.76 mmol, 93.74% yield) as a brown oil.

LCMS (ESI): [M+H]⁺m/z: calcd 313.16; found 313.2; Rt=1.083 min.

### Step 3: N′-benzyl-N′-[(4-methyl-2-pyridyl)methyl]oxamide

Ethyl 2-[benzyl-[(4-methyl-2-pyridyl)methyl]amino]-2-oxo-acetate (1.8 g, 5.76 mmol) was dissolved in MeOH (20 mL) saturated with NH3 (5.76 mmol) and stirred overnight. The RM was concentrated, redissolved in DCM, washed with water, dried over anhydrous sodium sulfate and concentrated in vacuo to give N′-benzyl-N′-[(4-methyl-2-pyridyl)methyl]oxamide (1.37 g, 4.84 mmol, 83.91% yield) as a brown oil.

LCMS (ESI): [M+H]⁺m/z: calcd 284.14; found 284.0; Rt=0.864 min.

### Step 4: N-[4-amino-1-(2-trimethylsilylethoxymethyl)pyrazolo[4,3-c]pyridin-7-yl]-N′-benzyl-N′-[(4-methyl-2-pyridyl)methyl]oxamide

Copper (4.49 mg, 70.59 μmol), Copper (I) iodide (40.33 mg, 211.77 μmol, 7.18 μL), cesium carbonate (460.00 mg, 1.41 mmol) were added to a stirred solution of N′-benzyl-N′-[(4-methyl-2-pyridyl)methyl]oxamide (200 mg, 705.90 μmol), 7-bromo-1-(2-trimethylsilylethoxymethyl)pyrazolo[4,3-c]pyridin-4-amine (290.80 mg, 847.09 μmol) and rac-(1R,2R)—N1,N2-dimethylcyclohexane-1,2-diamine (50.20 mg, 352.95 μmol) in 1,4-dioxane (5.00 mL) under Ar atmosphere and stirred at 100° C. for 12 hr in closed vial. The reaction mixture was filtered and evaporated in vacuo to give N-[4-amino-1-(2-trimethylsilylethoxymethyl)pyrazolo[4,3-c]pyridin-7-yl]-N′-benzyl-N′-[(4-methyl-2-pyridyl)methyl]oxamide (0.55 g, crude) as a brown gum.

LCMS (ESI): [M+H]⁺m/z: calcd 564.27; found 564.0; Rt=1.216 min.

### Step 5: The synthesis of N-(4-amino-1H-pyrazolo[4,3-c]pyridin-7-yl)-N′-benzyl-N′-[(4-methyl-2-pyridyl)methyl]oxamide

N-[4-amino-1-(2-trimethylsilylethoxymethyl)pyrazolo[4,3-c]pyridin-7-yl]-N′-benzyl-N′-[(4-methyl-2-pyridyl)methyl]oxamide (550 mg, 433.38 μmol) was dissolved in Dioxane (4 mL) saturated with HCl (10% by weight). MeOH (4 mL) was added for better solubility. The RM was stirred for 12 hr at 20° C. then concentrated in vacuo. The residue was dissolved in 3 mL of MeOH and subjected to HPLC (Device (Mobile Phase, Column): 0.6-6.6 min 30-80% H₂O-ACN+FA) to afford N-(4-amino-1H-pyrazolo[4,3-c]pyridin-7-yl)-N′-benzyl-N′-[(4-methyl-2-pyridyl)methyl]oxamide (60 mg, 130.02 μmol, 30.00% yield, HCOOH) was obtained as a yellow solid.

¹H NMR (600 MHz, DMSO-d₆) δ 2.10-2.32 (m, 3H), 4.11-4.89 (m, 4H), 6.04-7.04 (m, 2H), 7.07-7.22 (m, 2H), 7.26-7.66 (m, 5H), 7.66-8.14 (m, 1H), 8.14-8.20 (m, 1H), 8.30-8.39 (m, 1H), 9.71-10.68 (m, 1H), 12.47-13.47 (m, 1H).

LCMS (ESI): [M+H]⁺m/z: calcd 415.2; found 415.2; Rt=1.986 min.

### Example 94. Synthesis of N-(4-amino-1H-pyrazolo[4,3-c]pyridin-7-yl)-N′-benzyl-N′-[(4-chloro-2-methyl-phenyl)methyl]oxamide (Compound 186)

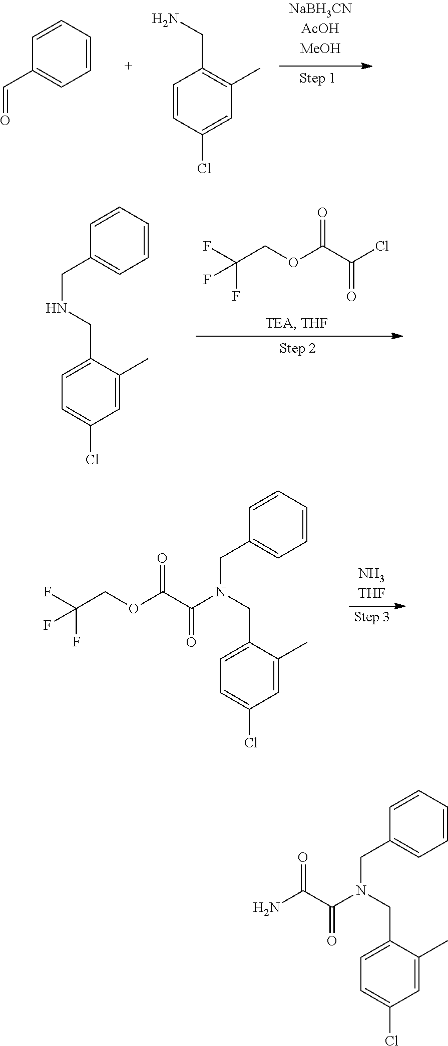

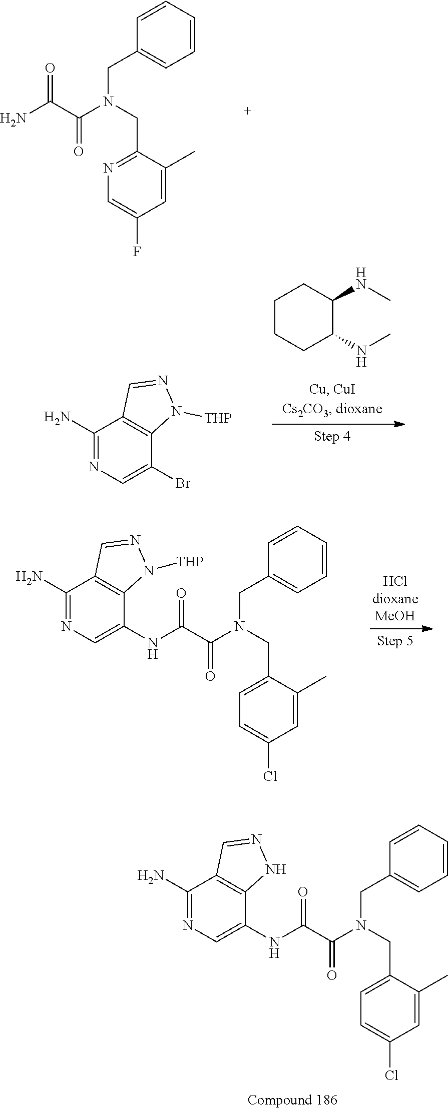

### Step 1: N-[(4-chloro-2-methyl-phenyl)methyl]-1-phenyl-methanamine

Potassium acetate (246.78 mg, 2.51 mmol, 157.18 μL) was added to a suspension of benzaldehyde (127.07 mg, 1.20 mmol) in MeOH (10 mL) and stirred for 30 min. (4-chloro-2-methyl-phenyl)methanamine (0.23 g, 1.20 mmol, 200.00 μL, HCl) was added and the reaction mixture was stirred for 30 min. Sodium cyanoborohydride (112.87 mg, 1.80 mmol) was added to the reaction mixture and stirred at 20° C. for 3 hr, Solvent was evaporated, NaOH (3 mL, 20%) was added to the residue and extracted with DCM (2×10 mL), combined extract was dried over anhydrous sodium sulfate, filtered and concentrated on vacuo to give N-[(4-chloro-2-methyl-phenyl)methyl]-1-phenyl-methanamine (0.21 g, 854.54 μmol, 71.37% yield) as a colorless liquid which was used in the next step without further purification.

LCMS (ESI): [M+H]⁺m/z: calcd 246.11; found 246.2; Rt=0.847 min.

### Step 2:2,2,2-trifluoroethyl 2-[benzyl-[(4-chloro-2-methyl-phenyl)methyl]amino]-2-oxo-acetate

2,2,2-trifluoroethyl 2-chloro-2-oxo-acetate (195.35 mg, 1.03 mmol) was added dropwise to a stirred solution of N-[(4-chloro-2-methyl-phenyl)methyl]-1-phenyl-methanamine (0.21 g, 854.54 μmol) and TEA (129.71 mg, 1.28 mmol, 178.66 μL) in THF (11.85 mL) at 0° C., stirred for 1 hr at 0° C. Reaction mixture was used in the next step.

LCMS (ESI): [M+H]⁺m/z: calcd 400.09; found 400.2; Rt=1.331 min.

### Step 3: N′-benzyl-N′-[(4-chloro-2-methyl-phenyl)methyl]oxamide

Ammonia (289.67 mg, 17.01 mmol) was bubbled trough a reaction mixture at 0° C. and stirred for 1 hr at 20° C. Reaction mixture was filtered, solid washed with THF (2×5 mL), filtrate concentrated in vacuo to give N′-benzyl-N′-[(4-chloro-2-methyl-phenyl)methyl]oxamide (0.26 g, 820.76 μmol, 96.51% yield) as a yellow gum.

LCMS (ESI): [M+Na]⁺m/z: calcd 339.10; found 339.0; Rt=1.073 min.

### Step 4: N-(4-amino-1-tetrahydropyran-2-yl-pyrazolo[4,3-c]pyridin-7-yl)-N′-benzyl-N′-[(4-chloro-2-methyl-phenyl)methyl]oxamide

Copper (2.61 mg, 41.04 μmol), Copper (I) iodide (78.16 mg, 410.38 μmol, 13.91 μL), cesium carbonate (534.84 mg, 1.64 mmol) was added to a stirred solution of N′-benzyl-N′-[(4-chloro-2-methyl-phenyl)methyl]oxamide (0.26 g, 820.76 μmol), 7-bromo-1-tetrahydropyran-2-yl-pyrazolo[4,3-c]pyridin-4-amine (280.47 mg, 943.87 μmol), rac-(1R,2R)—N1,N2-dimethylcyclohexane-1,2-diamine (58.37 mg, 410.38 μmol) in 1,4-dioxane (7 mL) under Ar atmosphere and stirred at 110° C. for 48 hr in closed vial. Reaction mixture was filtered, solid washed with dioxane (2×3 mL), filtrate concentrated to give crude N-(4-amino-1-tetrahydropyran-2-yl-pyrazolo[4,3-c]pyridin-7-yl)-N′-benzyl-N′-[(4-chloro-2-methyl-phenyl)methyl]oxamide (0.43 g, 806.72 μmol, 98.29% yield) as a brown solid.

LCMS (ESI): [M+H]⁺m/z: calcd 533.21; found 533.2; Rt=1.203 min.

### Step 5: The synthesis of N-(4-amino-1H-pyrazolo[4,3-c]pyridin-7-yl)-N′-benzyl-N′-[(4-chloro-2-methyl-phenyl)methyl]oxamide

Hydrogen chloride solution 4.0M in dioxane (2.94 g, 80.67 mmol, 3.68 mL) was added to a solution of N-(4-amino-1-tetrahydropyran-2-yl-pyrazolo[4,3-c]pyridin-7-yl)-N′-benzyl-N′-[(4-chloro-2-methyl-phenyl)methyl]oxamide (0.43 g, 806.72 μmol) in Methanol (4 mL) and stirred at 20° C. for 1 hr. Volatiles was evaporated in vacuo, the residue triturated with IPA (5 mL), filtered, washed with IPA (5 mL), and submitted to HPLC (column: Chromatorex 18 SMB100-5T 100×19 mm 5 μm; 10-25% 0-5 min H₂O/ACN/0.1% FA, flow rate: 30 mL/min) to give N-(4-amino-1H-pyrazolo[4,3-c]pyridin-7-yl)-N′-benzyl-N′-[(4-chloro-2-methyl-phenyl)methyl]oxamide (21 mg, 43.27 μmol, 5.36% yield, HCl) as a brown solid.

¹H NMR (600 MHz, DMSO-d₆) δ 1.77-2.16 (m, 3H), 4.36-4.52 (m, 2H), 4.70 (s, 2H), 6.20-7.00 (m, 2H), 7.00-7.14 (m, 1H), 7.21-7.29 (m, 4H), 7.31-7.36 (m, 3H), 7.57-7.65 (m, 1H), 8.04-8.28 (m, 2H), 9.69-10.72 (m, 1H), 12.66-13.45 (m, 1H).

LCMS (ESI): [M+H]⁺m/z: calcd 449.17; found 449.2; Rt=3.225 min.

### Example 95. Synthesis of N-(4-amino-1H-pyrazolo[4,3-c]pyridin-7-yl)-N′-benzyl-N′-[(4-fluoro-2-methyl-phenyl)methyl]oxamide (Compound 149)

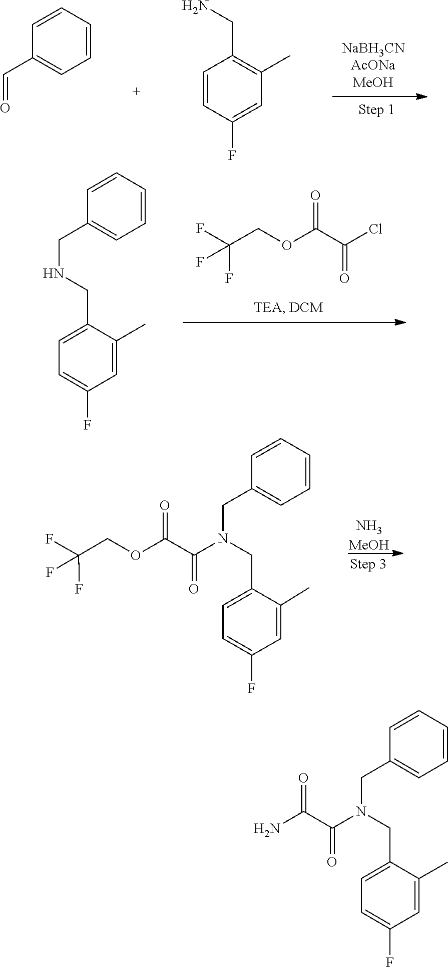

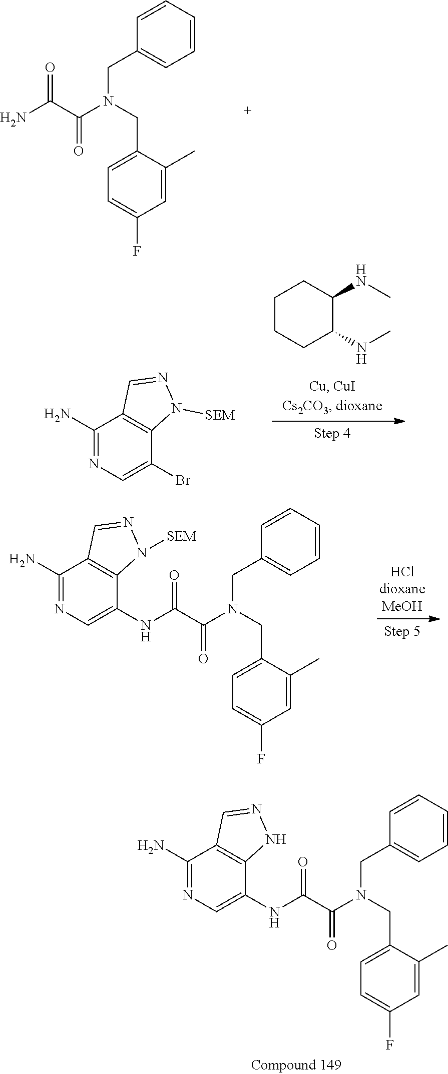

### Step 1: N-[(4-fluoro-2-methyl-phenyl)methyl]-1-phenyl-methanamine

4-fluoro-2-methyl-benzaldehyde (1 g, 7.24 mmol, 874.13 μL) and phenylmethanamine (775.69 mg, 7.24 mmol) were dissolved in MeOH (19.22 mL) and Sodium acetate (1.19 g, 14.48 mmol, 777.29 μL) was added thereto. The resulting mixture was stirred for 1 hr and Sodium cyanoborohydride (545.90 mg, 8.69 mmol) was added thereto. The resulting mixture was stirred overnight. The reaction mixture was concentrated in vacuo and aq. K₂CO₃ solution (15 mL) was added to the residue. The resulting mixture was extracted with DCM (2×10 mL) and combined organic layers were dried over anhydrous sodium sulfate, filtered and concentrated in vacuo to obtain N-[(4-fluoro-2-methyl-phenyl)methyl]-1-phenyl-methanamine (1.2 g, 5.23 mmol, 72.29% yield). as a light-yellow oil.

LCMS (ESI): [M+H]⁺m/z: calcd 230.14; found 230.2; Rt=0.812 min.

### Step 2:2,2,2-trifluoroethyl 2-[benzyl-[(4-fluoro-2-methyl-phenyl)methyl]amino]-2-oxo-acetate

N-[(4-fluoro-2-methyl-phenyl)methyl]-1-phenyl-methanamine (1.2 g, 5.23 mmol) was dissolved in DCM (28.18 mL) and TEA (1.32 g, 13.08 mmol, 1.82 mL) was added. The reaction mixture was cooled and 2,2,2-trifluoroethyl 2-chloro-2-oxo-acetate (1.20 g, 6.28 mmol) was added dropwise. After that it was stirred at rt overnight. Then water (10 mL) was added, organic layer was stirred with Brine and dried over anhydrous sodium sulfate, evaporated in vacuum to afford 2,2,2-trifluoroethyl 2-[benzyl-[(4-fluoro-2-methyl-phenyl)methyl]amino]-2-oxo-acetate (1.5 g, 3.91 mmol, 74.77% yield) as a light-yellow oil.

LCMS (ESI): [M+H]⁺m/z: calcd 384.12; found 384.2; Rt=1.528 min.

### Step 3: N′-benzyl-N′-[(4-fluoro-2-methyl-phenyl)methyl]oxamide

2,2,2-trifluoroethyl 2-[benzyl-[(4-fluoro-2-methyl-phenyl)methyl]amino]-2-oxo-acetate (1.5 g, 3.91 mmol) was dissolved in NH3/MeOH (20 mL) and stirred overnight at rt. Then it was evaporated in vacuum and subjected to CC (CHCl₃-MeCN was used as an eluent mixture) to afford N′-benzyl-N′-[(4-fluoro-2-methyl-phenyl)methyl]oxamide (0.7 g, 2.33 mmol, 59.57% yield) as a beige solid.

LCMS (ESI): [M+Na]⁺m/z: calcd 323.13; found 323.0; Rt=1.305 min.

### Step 4: N-[4-amino-1-(2-trimethylsilylethoxymethyl)pyrazolo[4,3-c]pyridin-7-yl]-N′-benzyl-N′-[(4-fluoro-2-methyl-phenyl)methyl]oxamide

A mixture of 7-bromo-1-(2-trimethylsilylethoxymethyl)pyrazolo[4,3-c]pyridin-4-amine (228.61 mg, 665.94 μmol), N′-benzyl-N′-[(4-fluoro-2-methyl-phenyl)methyl]oxamide (0.2 g, 665.94 μmol), Cesium carbonate (325.46 mg, 998.91 μmol), rac-(1R,2R)—N1,N2-dimethylcyclohexane-1,2-diamine (75.78 mg, 532.75 μmol) and CuI (76.10 mg, 399.56 μmol, 13.54 μL) with a few mg of Cu(2.12 mg, 33.30 μmol) in dioxane (3.99 mL) was stirred in a sealed vial under argon at 110° C. for 48 hr. The resulting mixture was cooled down and filtered through silica gel to afford N-[4-amino-1-(2-trimethylsilylethoxymethyl)pyrazolo[4,3-c]pyridin-7-yl]-N′-benzyl-N′-[(4-fluoro-2-methyl-phenyl)methyl]oxamide (0.37 g, 657.53 μmol, 98.74% yield) as a brown gum.

LCMS (ESI): [M+H]⁺m/z: calcd 563.26; found 563.2; Rt=1.491 min.

### Step 5: The synthesis of N-(4-amino-1H-pyrazolo[4,3-c]pyridin-7-yl)-N′-benzyl-N′-[(4-fluoro-2-methyl-phenyl)methyl]oxamide

N-[4-amino-1-(2-trimethylsilylethoxymethyl)pyrazolo[4,3-c]pyridin-7-yl]-N′-benzyl-N′-[(4-fluoro-2-methyl-phenyl)methyl]oxamide (0.37 g, 657.53 μmol) was dissolved in MeOH (5 mL) and dioxane/HCl (657.53 μmol, 3 mL) was added. Then mixture was stirred at rt 12 hr. The solution was evaporated and submitted to reverse phase HPLC (column: SunFire C18 100×19 mm, 5 μm; mobile phase: 20-45% 2-7.5 min water-MeCN+HCl, flow rate: 30 mL/min) to afford N-(4-amino-1H-pyrazolo[4,3-c]pyridin-7-yl)-N′-benzyl-N′-[(4-fluoro-2-methyl-phenyl)methyl]oxamide (37.9 mg, 80.83 μmol, 12.29% yield, HCl) as a beige solid.

¹H NMR (600 MHz, DMSO-d₆) δ 2.00-2.22 (m, 3H), 4.28-4.51 (m, 2H), 4.51-4.74 (m, 2H), 6.93-7.09 (m, 2H), 7.16-7.35 (m, 6H), 7.86-8.04 (m, 1H), 8.55-9.06 (m, 2H), 10.95-11.14 (m, 1H), 11.98-13.05 (m, 1H), 13.91-15.04 (m, 1H).

LCMS (ESI): [M+H]⁺m/z: calcd 433.2; found 433.2; Rt=2.692 min.

### Example 96. Synthesis of N-(4-amino-1H-pyrazolo[4,3-c]pyridin-7-yl)-N′-ethyl-N′-(o-tolylmethyl)oxamide (Compound 151)

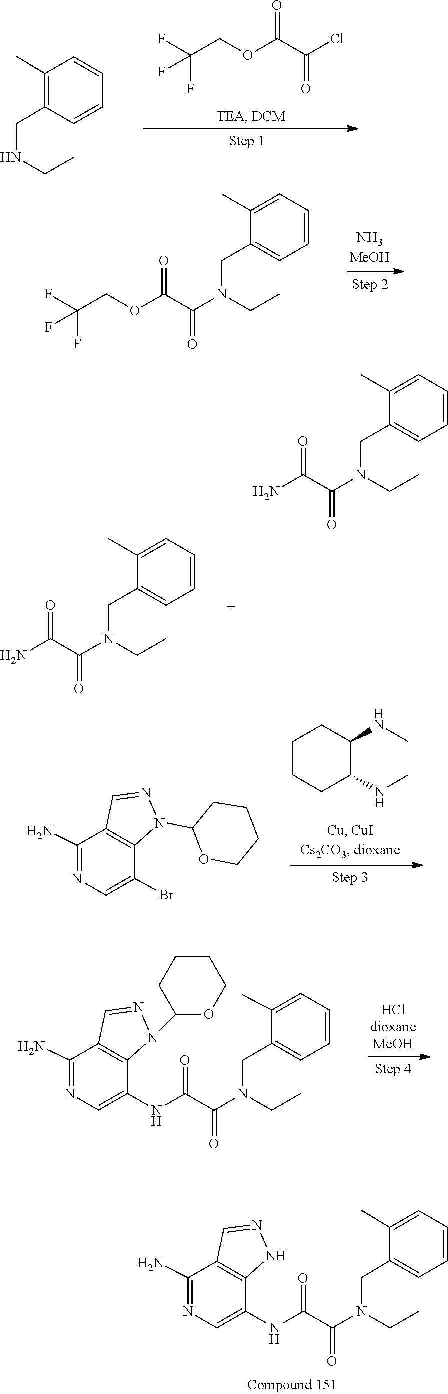

### Step 1:2,2,2-trifluoroethyl 2-[ethyl(o-tolylmethyl)amino]-2-oxo-acetate

N-(o-tolylmethyl)ethanamine (500 mg, 3.35 mmol) was dissolved in DCM (7 mL) and Triethylamine (372.94 mg, 3.69 mmol, 513.69 μL) was added thereto. The resulting mixture was cooled to −5° C. in an ice/methanol bath and a solution of 2,2,2-trifluoroethyl 2-chloro-2-oxo-acetate (670.20 mg, 3.52 mmol) in DCM (5 mL) was added dropwise at −5° C. After the addition was completed, the resulting mixture was allowed to warm to room temperature and stirred overnight. Water (25 mL) was added to the reaction mixture and an organic layer was separated. The aqueous layer was extracted with DCM (25 mL) and combined organic layers were dried over anhydrous sodium sulfate, filtered and concentrated in vacuo to obtain 2,2,2-trifluoroethyl 2-[ethyl(o-tolylmethyl)amino]-2-oxo-acetate (961 mg, 3.17 mmol, 94.58% yield) as a red oil.

LCMS (ESI): [M+H]⁺m/z: calcd 304.12; found 304.0; Rt=1.306 min.

### Step 2: N′-ethyl-N′-(o-tolylmethyl)oxamide

2,2,2-trifluoroethyl 2-[ethyl(o-tolylmethyl)amino]-2-oxo-acetate (961 mg, 3.17 mmol) was dissolved in MeOH (5 mL) and NH3/MeOH (20 mL) was added thereto. The resulting mixture was stirred overnight. The reaction mixture was concentrated in vacuo to obtain N′-ethyl-N′-(o-tolylmethyl)oxamide (753 mg, crude) as a yellow gum.

LCMS (ESI): [M+H]⁺m/z: calcd 221.13; found 221.1; Rt=1.074 min.

### Step 3: N-(4-amino-2-tetrahydropyran-2-yl-pyrazolo[4,3-c]pyridin-7-yl)-N′-ethyl-N′-(0-tolylmethyl)oxamide

To an 8 mL vial N′-ethyl-N′-(o-tolylmethyl)oxamide (155 mg, 703.69 μmol), 7-bromo-2-tetrahydropyran-2-yl-pyrazolo[4,3-c]pyridin-4-amine (230.01 mg, 774.06 μmol), Copper (2.24 mg, 35.18 μmol), Copper (I) iodide (67.01 mg, 351.85 μmol, 11.92 μL), rac-(1R,2R)—N1,N2-dimethylcyclohexane-1,2-diamine (75.07 mg, 527.77 μmol), Cesium carbonate (458.55 mg, 1.41 mmol) and Dioxane (3 mL) were charged. The resulting mixture was splurged with argon for 5 min. The vial was sealed and heated at 110° C. for 46 hr. The reaction mixture was cooled and filtered. The filter cake was rinsed with dioxane (10 mL) and the filtrate was concentrated in vacuo. The residue was submitted to HPLC and purified (0-2-10 min, 43-50-85% H₂O/MeOH/0.1% NH₄OH, flow 30 mL/min ((loading pump 4 mL MeOH), column: Chromatorex SMB100-5T C18 100×19 mm, 5 microM) to obtain N-(4-amino-2-tetrahydropyran-2-yl-pyrazolo[4,3-c]pyridin-7-yl)-N′-ethyl-N′-(o-tolylmethyl)oxamide (25 mg, 57.27 μmol, 8.14% yield). The filter cake was dissolved in DMSO(4 mL), filtered, the filtrate was submitted to HPLC and purified (0-2-10 min, 43-50-85% H₂O/MeOH/0.1% NH₄OH, flow 30 mL/min ((loading pump 4 mL MeOH), column: Chromatorex SMB100-5T C18 100×19 mm, 5 microM) to obtain N-(4-amino-2-tetrahydropyran-2-yl-pyrazolo[4,3-c]pyridin-7-yl)-N′-ethyl-N′-(o-tolylmethyl)oxamide (16.2 mg, 37.11 μmol, 5.27% yield) as a light-yellow solid.

LCMS (ESI): [M+H]⁺m/z: calcd 437.26; found 437.2; Rt=1.153 min.

### Step 4: The synthesis of N-(4-amino-1H-pyrazolo[4,3-c]pyridin-7-yl)-N′-ethyl-N′-(0-tolylmethyl)oxamide

N-(4-amino-2-tetrahydropyran-2-yl-pyrazolo[4,3-c]pyridin-7-yl)-N′-ethyl-N′-(o-tolylmethyl)oxamide (25 mg, 57.27 μmol) was dissolved in MeOH (1 mL) and HCl/dioxane (1 mL) was added thereto. The resulting mixture was stirred for 1 hr and then concentrated in vacuo. The residue was combined with the residue of a separate batch and purified by HPLC (0-2-10 min, 0-0-90% H₂O/MeOH/0.1% formic acid, flow 30 mL/min ((loading pump 4 mL MeOH), column: Chromatorex SMB100-5T C18 100×19 mm, 5 microM) to obtain N-(4-amino-1H-pyrazolo[4,3-c]pyridin-7-yl)-N′-ethyl-N′-(o-tolylmethyl)oxamide (15.8 mg, 39.66 μmol, 69.24% yield, HCOOH) as a light-yellow solid.

¹H NMR (600 MHz, DMSO-d₆) δ 0.90-1.17 (m, 3H), 2.01-2.31 (m, 3H), 3.27-3.29 (m, 1H), 3.43-3.45 (m, 1H), 4.26-4.83 (m, 2H), 6.58-6.88 (m, 2H), 7.15-7.24 (m, 3H), 7.47-7.75 (m, 1H), 8.08-8.83 (m, 2H), 9.42-10.61 (m, 1H), 12.35-13.63 (m, 1H).

LCMS (ESI): [M+H]⁺m/z: calcd 353.19; found 353.0; Rt=0.933 min.

### Example 97. Synthesis of N-(4-amino-1H-pyrazolo[4,3-c]pyridin-7-yl)-N′-ethyl-N′-[(4-fluoro-2-methyl-phenyl)methyl]oxamide (Compound 166)

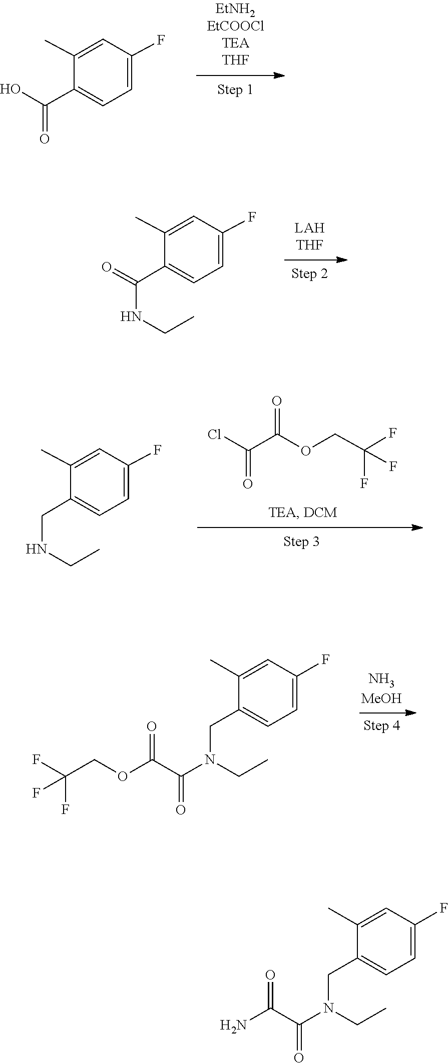

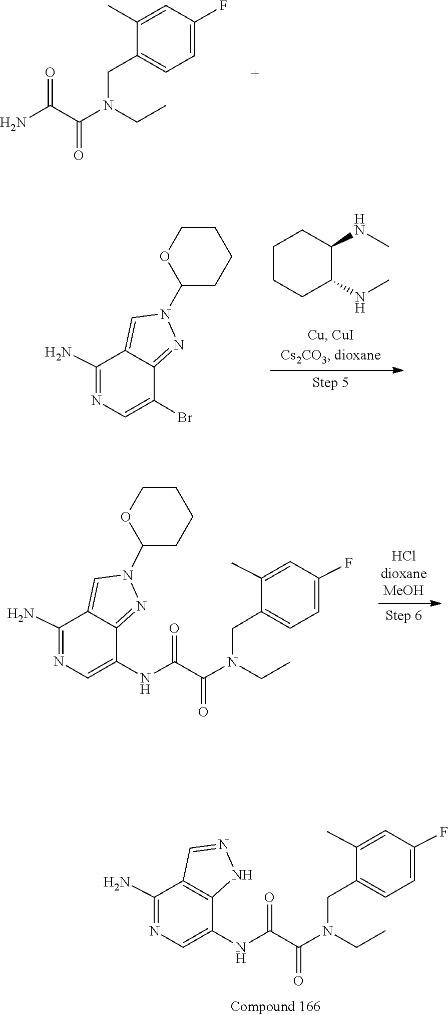

### Step 1: N-ethyl-4-fluoro-2-methyl-benzamide

4-fluoro-2-methyl-benzoic acid (1 g, 6.49 mmol) was dissolved in THF (7 mL) and Triethylamine (722.14 mg, 7.14 mmol, 994.68 μL) was added thereto. The resulting mixture was cooled to −5° C. and Ethyl chloroformate, 97% (739.27 mg, 6.81 mmol, 650.77 μL) was added dropwise to the previous solution. After the addition was completed, the resulting mixture was stirred for 15 min and Ethylamine, 70% aq. soln. (2.92 g, 45.41 mmol, 3.64 mL, 70% purity) was added. The resulting mixture was allowed to warm to room temperature and stirred overnight. The reaction mixture was concentrated in vacuo and the residue was diluted with water (25 mL). The resulting mixture was extracted with DCM (2×35 mL) and combined organic layers were washed with water (25 mL), dried over anhydrous sodium sulfate, filtered and concentrated in vacuo to obtain N-ethyl-4-fluoro-2-methyl-benzamide (0.65 g, 3.59 mmol, 55.29% yield) as a white solid which was used further without purification.

LCMS (ESI): [M+H]⁺m/z: calcd 182.1; found 182.2; Rt=0.827 min.

### Step 2: N-[(4-fluoro-2-methyl-phenyl)methyl]ethanamine

Lithium aluminum tetrahydride (680.72 mg, 17.94 mmol) was suspended in THF (20 mL) and the resulting suspension was heated to reflux. A solution of N-ethyl-4-fluoro-2-methyl-benzamide (0.65 g, 3.59 mmol) in THF (5 mL) was added dropwise to the previous suspension maintaining gentle reflux. after addition was completed, the resulting mixture was refluxed for 4 hr. The reaction mixture was cooled in an ice bath and water (0.68 mL) was carefully added dropwise followed by the addition of 15% KOH solution (0.68 mL) and water (1.36 mL). The resulting mixture was stirred for 20 min and filtered. The filtercake was rinsed with THF (20 mL) and the filtrate was concentrated in vacuo to obtain N-[(4-fluoro-2-methyl-phenyl)methyl]ethanamine (400 mg, 2.39 mmol, 66.68% yield) as a colorless liquid.

LCMS (ESI): [M+H]⁺m/z: calcd 168.12; found 168.2; Rt=0.409 min.

### Step 3:2,2,2-trifluoroethyl 2-[ethyl-[(4-fluoro-2-methyl-phenyl)methyl]amino]-2-oxo-acetate

N-[(4-fluoro-2-methyl-phenyl)methyl]ethanamine (400 mg, 2.39 mmol) and Triethylamine (266.25 mg, 2.63 mmol, 366.74 μL) were dissolved in DCM (7 mL) and the resulting mixture was cooled to −5° C. in an ice/methanol bath. A solution of 2,2,2-trifluoroethyl 2-chloro-2-oxo-acetate (478.48 mg, 2.51 mmol) in DCM (5 mL) was added dropwise at −5° C. After the addition was completed, the resulting mixture was allowed to warm to room temperature and stirred overnight. Water (15 mL) was added to the reaction mixture and an organic layer was separated. The aqueous layer was extracted with DCM (25 mL) and combined organic layers were dried over anhydrous sodium sulfate, filtered and concentrated in vacuo to obtain 2,2,2-trifluoroethyl 2-[ethyl-[(4-fluoro-2-methyl-phenyl)methyl]amino]-2-oxo-acetate (700 mg, 2.18 mmol, 91.09% yield) as a red oil.

LCMS (ESI): [M+H]⁺m/z: calcd 322.11; found 322.0; Rt=1.486 min.

### Step 4:2-[ethyl-[(4-fluoro-2-methyl-phenyl)methyl]amino]-2-oxo-acetic acid

2,2,2-trifluoroethyl 2-[ethyl-[(4-fluoro-2-methyl-phenyl)methyl]amino]-2-oxo-acetate (700 mg, 2.18 mmol) was dissolved in MeOH (5 mL) and NH3/MeOH (20 mL) was added thereto. The resulting mixture was stirred overnight. The reaction mixture was filtered and the filtrate was concentrated in vacuo to obtain 2-[ethyl-[(4-fluoro-2-methyl-phenyl)methyl]amino]-2-oxo-acetic acid (468 mg, 1.96 mmol, 89.78% yield) as a yellow gum.

LCMS (ESI): [M+Na]⁺m/z: calcd 261.11; found 261.0; Rt=1.016 min.

### Step 5: N-(4-amino-2-tetrahydropyran-2-yl-pyrazolo[4,3-c]pyridin-7-yl)-N′-ethyl-N′-[(4-fluoro-2-methyl-phenyl)methyl]oxamide

To an 8 ml vial N′-ethyl-N′-[(4-fluoro-2-methyl-phenyl)methyl]oxamide (162 mg, 679.94 μmol), 7-bromo-2-tetrahydropyran-2-yl-pyrazolo[4,3-c]pyridin-4-amine (222.25 mg, 747.93 μmol), Copper (2.16 mg, 34.00 μmol), Copper (I) iodide (64.75 mg, 339.97 μmol, 11.52 μL), rac-(1R,2R)—N1,N2-dimethylcyclohexane-1,2-diamine (72.54 mg, 509.95 μmol), Cesium carbonate (443.07 mg, 1.36 mmol) and Dioxane (3 mL) were charged and the resulting mixture was splurged with argon for 5 min. The vial was sealed and heated at 110° C. for 65 hr. The reaction mixture was cooled, diluted with MeOH (5 mL), and filtered. The filter cake was rinsed with MeOH (5 mL), and the filtrate was concentrated in vacuo. The residue was submitted to HPLC and purified (0-2-10 min, 43-55-85% H₂O/MeOH/0.1% NH₄OH, flow 30 mL/min ((loading pump 4 mL MeOH/0.1% NH₄OH), column: XBridge BEH C18 100×19 mm, 5 microM) to obtain N-(4-amino-2-tetrahydropyran-2-yl-pyrazolo[4,3-c]pyridin-7-yl)-N′-ethyl-N′-[(4-fluoro-2-methyl-phenyl)methyl]oxamide (19.3 mg, 42.46 μmol, 6.25% yield) as a yellow gum.

LCMS (ESI): [M+H]⁺m/z: calcd 455.25; found 455.2; Rt=1.079 min.

### Step 6: The synthesis of N-(4-amino-1H-pyrazolo[4,3-c]pyridin-7-yl)-N′-ethyl-N′-[(4-fluoro-2-methyl-phenyl)methyl]oxamide

N-(4-amino-2-tetrahydropyran-2-yl-pyrazolo[4,3-c]pyridin-7-yl)-N′-ethyl-N′-[(4-fluoro-2-methyl-phenyl)methyl]oxamide (19.7 mg, 43.34 μmol) was dissolved in MeOH (1 mL) and HCl/dioxane (1 mL) was added thereto. The resulting solution was stirred for 1 hr and the reaction mixture was concentrated in vacuo. The residue was purified by HPLC (0-2-10 min, 0-75% H₂O/MeCN/0.1% formic acid, flow 30 mL/min ((loading pump 4 mL MeCN), target mass 370, column: Chromatorex SMB100-5T C18 100×19 mm, 5 microM) to obtain N-(4-amino-1H-pyrazolo[4,3-c]pyridin-7-yl)-N′-ethyl-N′-[(4-fluoro-2-methyl-phenyl)methyl]oxamide (5.3 mg, 12.73 μmol, 29.36% yield, HCOOH) as a light-yellow gum.

LCMS (ESI): [M+H]⁺m/z: calcd 371.18; found 371.0; Rt=0.960 min.

### Example 98. Synthesis of N-(4-amino-1H-pyrazolo[4,3-c]pyridin-7-yl)-N′-[(4-fluorophenyl)methyl]-N′-[(3-methyl-2-pyridyl)methyl]oxamide (Compound 119)

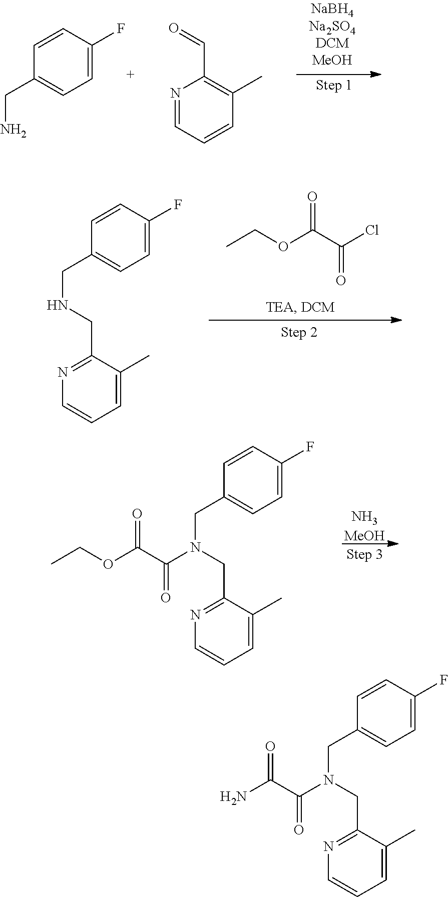

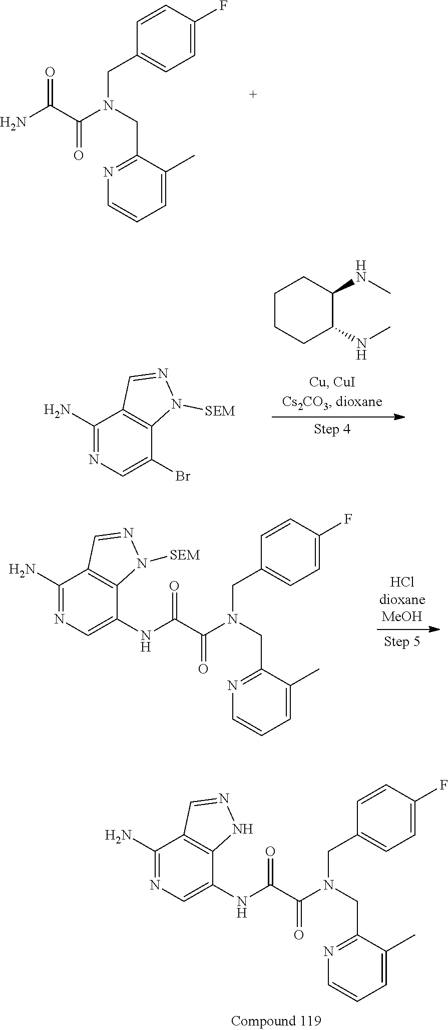

### Step 1:1-(4-fluorophenyl)-N-[(3-methyl-2-pyridyl)methyl]methanamine

(4-fluorophenyl)methanamine (0.95 g, 7.59 mmol, 867.58 μL), Sodium sulfate, anhydrous (1.08 g, 7.59 mmol, 402.34 μL) and 3-methylpyridine-2-carbaldehyde (919.58 mg, 7.59 mmol, 851.47 μL) were mixed in DCM at 20° C. The resulting mixture was stirred at 20° C. for 12 hr, then filtered and concentrated in vacuo. The residue was dissolved in methanol, cooled to 5° C. and Sodium Borohydride (315.90 mg, 8.35 mmol, 294.13 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford 1-(4-fluorophenyl)-N-[(3-methyl-2-pyridyl)methyl]methanamine (1.48 g, crude) as a brown liquid.

LCMS (ESI): [M+H]⁺m/z: calcd 231.13; found 231.0; Rt=0.723 min.

### Step 2: ethyl 2-[(4-fluorophenyl)methyl-[(3-methyl-2-pyridyl)methyl]amino]-2-oxo-acetate

1-(4-fluorophenyl)-N-[(3-methyl-2-pyridyl)methyl]methanamine (1.48 g, 3.86 mmol) and TEA (780.41 mg, 7.71 mmol, 1.07 mL) were dissolved in acetonitrile (21.09 mL), cooled with ice-water bath and then ethyl 2-chloro-2-oxoacetate (684.45 mg, 5.01 mmol, 560.10 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-[(4-fluorophenyl)methyl-[(3-methyl-2-pyridyl)methyl]amino]-2-oxo-acetate (1.93 g, crude) as a brown liquid.

LCMS (ESI): [M+H]⁺m/z: calcd 331.15; found 331.2; Rt=1.105 min.

### Step 3: N′-[(4-fluorophenyl)methyl]-N′-[(3-methyl-2-pyridyl)methyl]oxamide

Ethyl 2-[(4-fluorophenyl)methyl-[(3-methyl-2-pyridyl)methyl]amino]-2-oxo-acetate (1.93 g, 2.34 mmol) was dissolved in MeOH (20 mL) saturated with NH3 (2.34 mmol) and stirred overnight. The RM was concentrated, redissolved in DCM, washed with water, dried over anhydrous sodium sulfate and concentrated in vacuo to give N′-[(4-fluorophenyl)methyl]-N′-[(3-methyl-2-pyridyl)methyl]oxamide (1.05 g, crude) as a brown solid.

LCMS (ESI): [M+H]⁺m/z: calcd 302.13; found 302.0; Rt=0.891 min.

### Step 4: N-[4-amino-1-(2-trimethylsilylethoxymethyl)pyrazolo[4,3-c]pyridin-7-yl]-N′-[(4-fluorophenyl)methyl]-N′-[(3-methyl-2-pyridyl)methyl]oxamide

Copper (1.86 mg, 29.21 μmol), Copper (I) iodide (16.69 mg, 87.62 μmol, 2.97 μL), cesium carbonate (190.31 mg, 584.11 μmol) were added to a stirred solution of N′-[(4-fluorophenyl)methyl]-N′-[(3-methyl-2-pyridyl)methyl]oxamide (220 mg, 292.05 μmol), 7-bromo-1-(2-trimethylsilylethoxymethyl)pyrazolo[4,3-c]pyridin-4-amine (120.31 mg, 350.46 μmol) and rac-(1R,2R)—N1,N2-dimethylcyclohexane-1,2-diamine (20.77 mg, 146.03 μmol) in 1,4-dioxane (5.01 mL) under Ar atmosphere and stirred at 100° C. for 12 hr in closed vial. The reaction mixture was filtered and evaporated in vacuo to give N-[4-amino-1-(2-trimethylsilylethoxymethyl)pyrazolo[4,3-c]pyridin-7-yl]-N′-[(4-fluorophenyl)methyl]-N′-[(3-methyl-2-pyridyl)methyl]oxamide (0.45 g, crude) as a brown solid.

LCMS (ESI): [M+H]⁺m/z: calcd 564.26; found 564.2; Rt=1.510 min.

### Step 5: The synthesis of N-(4-amino-1H-pyrazolo[4,3-c]pyridin-7-yl)-N′-[(4-fluorophenyl)methyl]-N′-[(3-methyl-2-pyridyl)methyl]oxamide

N-[4-amino-1-(2-trimethylsilylethoxymethyl)pyrazolo[4,3-c]pyridin-7-yl]-N′-[(4-fluorophenyl)methyl]-N′-[(3-methyl-2-pyridyl)methyl]oxamide (0.45 g, 119.75 μmol) was dissolved in Dioxane (4 mL) saturated with HCl (10% by weight). MeOH (4 mL) was added for better solubility. The RM was stirred for 12 hr at 20° C. then concentrated in vacuo. The residue was dissolved in 3 mL of MeOH and subjected to HPLC (Device (Mobile Phase, Column): 2-2-6 min 25-55% MeOH+FA flow 30 mL/min) to afford N-(4-amino-1H-pyrazolo[4,3-c]pyridin-7-yl)-N′-[(4-fluorophenyl)methyl]-N′-[(3-methyl-2-pyridyl)methyl]oxamide (13.1 mg, 27.32 μmol, 22.82% yield, HCOOH) as a yellow solid.

¹H NMR (600 MHz, DMSO-d₆) δ 2.12-2.26 (m, 3H), 4.43-4.70 (m, 2H), 4.81-5.01 (m, 2H), 7.10-7.17 (m, 3H), 7.17-7.34 (m, 2H), 7.33-7.37 (m, 1H), 7.38-7.44 (m, 1H), 7.47-7.57 (m, 1H), 7.60-7.81 (m, 1H), 8.29 (s, 1H), 8.33-8.44 (m, 1H), 10.38-10.71 (m, 1H), 13.10 (br s, 1H).

LCMS (ESI): [M+H]⁺m/z: calcd 434.19; found 434.2; Rt=2.411 min.

### Example 99. Synthesis of N-(4-amino-1H-pyrazolo[4,3-c]pyridin-7-yl)-N′-isobutyl-N′-(0-tolylmethyl)oxamide (Compound 185)

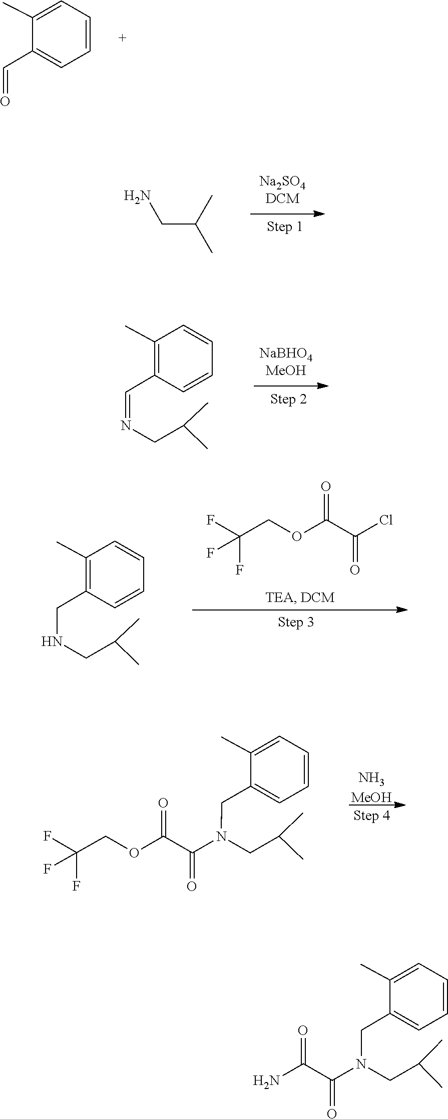

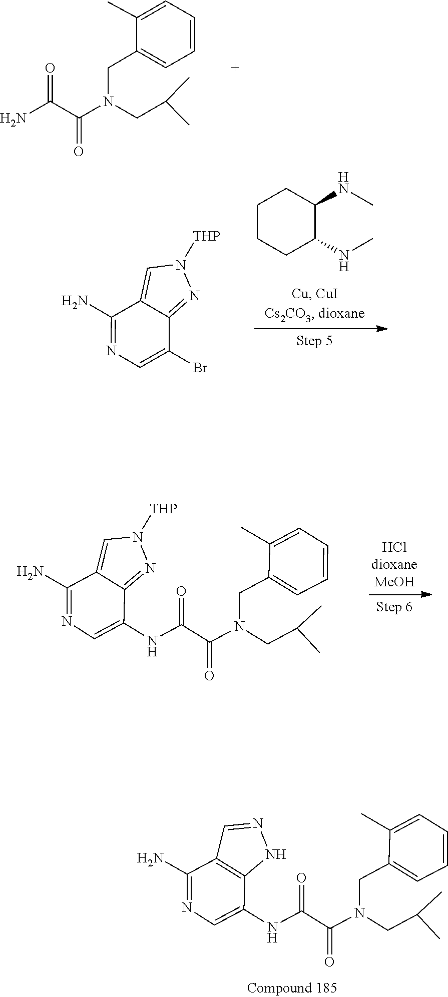

### Step 1: (Z)—N-isobutyl-1-(o-tolyl) methanimine

2-methylbenzaldehyde (500 mg, 4.16 mmol, 481.23 μL) was dissolved in DCM (8 mL) and 2-methylpropan-1-amine (304.36 mg, 4.16 mmol, 413.53 μL) was added thereto followed by the addition of Sodium sulfate, anhydrous (2.36 g, 16.65 mmol, 882.24 μL). The resulting mixture was vigorously stirred overnight. The reaction mixture was filtered and the filtrate was concentrated in vacuo to obtain (Z)—N-isobutyl-1-(o-tolyl) methanimine (723 mg, 4.13 mmol, 99.12% yield) as a colorless liquid.

### Step 2:2-methyl-N-(o-tolylmethyl)propan-1-amine

(Z)—N-isobutyl-1-(o-tolyl) methanimine (723 mg, 4.13 mmol) was dissolved in MeOH (10 mL) and Sodium Borohydride (468.19 mg, 12.38 mmol, 435.93 μL) was added portionwise. After the addition was completed, the reaction mixture was stirred for 1 hr. Water (5 mL) was added to the reaction mixture and the resulting mixture was concentrated in vacuo. Water (20 mL) was added to the residue and the resulting mixture was extracted with DCM (2×25 mL). Combined organic layers were dried over anhydrous sodium sulfate, filtered, and concentrated in vacuo to obtain 2-methyl-N-(o-tolylmethyl)propan-1-amine (578 mg, 3.26 mmol, 79.04% yield) as a colorless oil.

LCMS (ESI): [M+H]⁺m/z: calcd 178.16; found 178.2; Rt=0.700 min.

### Step 3:2,2,2-trifluoroethyl 2-[isobutyl(o-tolylmethyl)amino]-2-oxo-acetate

2-methyl-N-(o-tolylmethyl)propan-1-amine (578 mg, 3.26 mmol) and Triethylamine (362.90 mg, 3.59 mmol, 499.86 μL) were dissolved in DCM (10 mL) and the resulting solution was cooled to −5° C. in an ice/methanol bath. A solution of 2,2,2-trifluoroethyl 2-chloro-2-oxo-acetate (652.16 mg, 3.42 mmol) in DCM (3 mL) was added dropwise at −5° C. After the addition was completed, the resulting mixture was allowed to warm to room temperature and stirred overnight. Water (15 mL) was added to the reaction mixture and an organic layer was separated. The aqueous layer was extracted with DCM (25 mL) and combined organic layers were dried over anhydrous sodium sulfate, filtered and concentrated in vacuo to obtain 2,2,2-trifluoroethyl 2-[isobutyl(o-tolylmethyl)amino]-2-oxo-acetate (997 mg, 3.01 mmol, 92.30% yield) as a yellow gum.

LCMS (ESI): [M+H]⁺m/z: calcd 332.15; found 332.2; Rt=1.584 min.

### Step 4: N′-isobutyl-N′-(o-tolylmethyl)oxamide

2,2,2-trifluoroethyl 2-[isobutyl(o-tolylmethyl)amino]-2-oxo-acetate (997 mg, 3.01 mmol) was dissolved in MeOH (5 mL) and NH3/MeOH (20 mL) was added thereto. The resulting mixture was stirred overnight. The reaction mixture was filtered and the filtrate was concentrated in vacuo to obtain N′-isobutyl-N′-(o-tolylmethyl)oxamide (0.79 g, crude) as a yellow gum.

LCMS (ESI): [M+H]⁺m/z: calcd 249.16; found 249.0; Rt=1.151 min.

### Step 5: N-(4-amino-2-tetrahydropyran-2-yl-pyrazolo[4,3-c]pyridin-7-yl)-N′-isobutyl-N′-(0-tolylmethyl)oxamide

To an 8 ml vial N′-isobutyl-N′-(o-tolylmethyl)oxamide (154 mg, 620.17 μmol), 7-bromo-2-tetrahydropyran-2-yl-pyrazolo[4,3-c]pyridin-4-amine (202.71 mg, 682.18 μmol), Copper (1.97 mg, 31.01 μmol), Copper (I) iodide (59.06 mg, 310.08 μmol, 10.51 μL), rac-(1R,2R)—N1,N2-dimethylcyclohexane-1,2-diamine (66.16 mg, 465.13 μmol), Cesium carbonate (404.13 mg, 1.24 mmol) and Dioxane (3 mL) were charged and the resulting mixture was splurged with argon for 5 min. The vial was sealed and heated at 110° C. for 65 hr. The reaction mixture was cooled, diluted with MeOH (5 mL), and filtered. The filtercake was rinsed with MeOH (5 mL) and the filtrate was concentrated in vacuo. The residue was submitted to HPLC and purified (0-2-10 min, 43-55-85% H₂O/MeOH/0.1% NH₄OH, flow 30 mL/min ((loading pump 4 mL MeOH/0.1% NH₄OH), column: XBridge BEH C18 100×19 mm, 5 microM) to obtain N-(4-amino-2-tetrahydropyran-2-yl-pyrazolo[4,3-c]pyridin-7-yl)-N′-isobutyl-N′-(o-tolylmethyl)oxamide (39.7 mg, 85.46 μmol, 13.78% yield) as a yellow gum.

LCMS (ESI): [M+H]⁺m/z: calcd 465.3; found 465.2; Rt=1.163 min.

### Step 6: The synthesis of N-(4-amino-1H-pyrazolo[4,3-c]pyridin-7-yl)-N′-isobutyl-N′-(0-tolylmethyl)oxamide

N-(4-amino-2-tetrahydropyran-2-yl-pyrazolo[4,3-c]pyridin-7-yl)-N′-isobutyl-N′-(o-tolylmethyl)oxamide (39.7 mg, 85.46 μmol) was dissolved in MeOH (1 mL) and HCl/dioxane (1 mL) was added thereto. The resulting solution was stirred for 1 hr and the reaction mixture was concentrated in vacuo. The residue was purified by HPLC (0-2-10 min, 0-75% H₂O/MeCN/0.1% FA, flow 30 mL/min ((loading pump 4 mL MeCN), target mass 380, column: Chromatorex SMB100-5T C18 100×19 mm, 5 microM) to obtain N-(4-amino-1H-pyrazolo[4,3-c]pyridin-7-yl)-N′-isobutyl-N′-(o-tolylmethyl)oxamide (10.5 mg, 24.62 μmol, 28.81% yield, HCOOH) as a light-yellow gum.

¹H NMR (600 MHz, DMSO-d₆) δ 0.79-0.89 (m, 6H), 1.93-2.02 (m, 1H), 2.20-2.31 (m, 3H), 3.07-3.11 (m, 1H), 3.27-3.28 (m, 1H), 4.57-4.83 (m, 2H), 6.64 (s, 1H), 6.68 (s, 1H), 7.12-7.16 (m, 1H), 7.17-7.23 (m, 3H), 7.51-7.74 (m, 1H), 8.14-8.21 (m, 1H), 10.36-10.53 (m, 1H), 12.37-13.45 (m, 1H).

LCMS (ESI): [M+H]⁺m/z: calcd 381.23; found 381.2; Rt=1.114 min.

### Example 100. Synthesis of N-(4-amino-1H-pyrazolo[4,3-c]pyridin-7-yl)-N′-benzyl-N′-[[3-(trifluoromethyl)-2-pyridyl]methyl]oxamide (Compound 154)

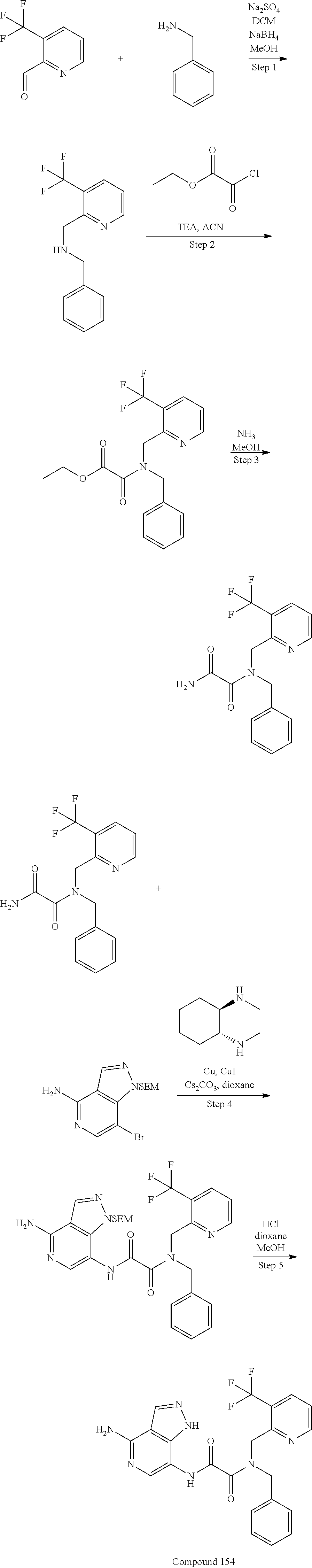

### Step 1:1-phenyl-N-[[3-(trifluoromethyl)-2-pyridyl]methyl]methanamine

Phenylmethanamine (0.7 g, 6.53 mmol), Sodium sulfate, anhydrous (927.91 mg, 6.53 mmol, 346.23 μL) and 3-(trifluoromethyl)pyridine-2-carbaldehyde (1.14 g, 6.53 mmol) were mixed in DCM at 20° C. The resulting mixture was stirred at 20° C. for 12 hr, then filtered and concentrated in vacuo. The residue was dissolved in methanol, cooled to 5° C. and Sodium Borohydride (271.85 mg, 7.19 mmol, 253.12 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford 1-phenyl-N-[[3-(trifluoromethyl)-2-pyridyl]methyl]methanamine (1.3 g, crude) as a brown oil.

LCMS (ESI): [M+H]⁺m/z: calcd 267.11; found 267.2; Rt=0.923 min.

### Step 2: ethyl 2-[benzyl-[[3-(trifluoromethyl)-2-pyridyl]methyl]amino]-2-oxo-acetate

1-phenyl-N-[[3-(trifluoromethyl)-2-pyridyl]methyl]methanamine (1.30 g, 4.90 mmol) and TEA (991.45 mg, 9.80 mmol, 1.37 mL) were dissolved in acetonitrile (20 mL), cooled with ice-water bath and then ethyl 2-chloro-2-oxoacetate (869.54 mg, 6.37 mmol, 711.57 μL) was added. Solution was stirred overnight at 20° C. The RM was concentrated in vacuo, dissolved in DCM (40 mL), and washed with water twice. Organic phase was dried over anhydrous sodium sulfate and concentrated in vacuo to give ethyl 2-[benzyl-[[3-(trifluoromethyl)-2-pyridyl]methyl]amino]-2-oxo-acetate (1.7 g, 4.64 mmol, 94.73% yield) as a dark-red liquid.

LCMS (ESI): [M+H]⁺m/z: calcd 367.13; found 367.2; Rt=1.279 min.

### Step 3: N′-benzyl-N′-[[3-(trifluoromethyl)-2-pyridyl]methyl]oxamide

Ethyl 2-[benzyl-[[3-(trifluoromethyl)-2-pyridyl]methyl]amino]-2-oxo-acetate (1.7 g, 4.64 mmol) was dissolved in MeOH (20 mL) saturated with NH3 (4.64 mmol) and stirred overnight. The RM was concentrated, dissolved in DCM, washed with water, dried over sodium sulphate and concentrated in vacuo to give N′-benzyl-N′-[[3-(trifluoromethyl)-2-pyridyl]methyl]oxamide (1.25 g, crude) as a brown solid.

LCMS (ESI): [M+H]⁺m/z: calcd 338.11; found 338.0; Rt=1.172 min.

### Step 4: N-[4-amino-1-(2-trimethylsilylethoxymethyl)pyrazolo[4,3-c]pyridin-7-yl]-N′-benzyl-N′-[[3-(trifluoromethyl)-2-pyridyl]methyl]oxamide

Copper (4.15 mg, 65.22 μmol, Copper (I) iodide (37.27 mg, 195.67 μmol, 6.63 μL), cesium carbonate (425.03 mg, 1.30 mmol) were added to a stirred solution of 7-bromo-1-(2-trimethylsilylethoxymethyl)pyrazolo[4,3-c]pyridin-4-amine (268.69 mg, 782.70 μmol), N′-benzyl-N′-[[3-(trifluoromethyl)-2-pyridyl]methyl]oxamide (220 mg, 652.25 μmol) and rac-(1R,2R)—N1,N2-dimethylcyclohexane-1,2-diamine (46.39 mg, 326.12 μmol) in 1,4-dioxane (5.00 mL) under Ar atmosphere and stirred at 100° C. for 12 hr in closed vial. The reaction mixture was filtered and evaporated in vacuo to give N-[4-amino-1-(2-trimethylsilylethoxymethyl)pyrazolo[4,3-c]pyridin-7-yl]-N′-benzyl-N′-[3-(trifluoromethyl)-2-pyridyl]methyl]oxamide (0.5 g, crude) as a dark-brown solid.

LCMS (ESI): [M−H]⁻ m/z: calcd 598.22; found 598.1; Rt=1.599 min.

### Step 5: The synthesis of N-(4-amino-1H-pyrazolo[4,3-c]pyridin-7-yl)-N′-benzyl-N′-[[3-(trifluoromethyl)-2-pyridyl]methyl]oxamide

N-[4-amino-1-(2-trimethylsilylethoxymethyl)pyrazolo[4,3-c]pyridin-7-yl]-N′-benzyl-N′-[[3-(trifluoromethyl)-2-pyridyl]methyl]oxamide (0.5 g, 283.49 μmol) was dissolved in Dioxane (4 mL) saturated with HCl (10% by weight). MeOH (4 mL) was added for better solubility. The RM was stirred for 12 hr at 20° C. then concentrated in vacuo. The residue was dissolved in 3 mL of MeOH and subjected to HPLC (Device (Mobile Phase, Column): 2-2-6 min 30-60% MeOH+FA, flow 30 mL/min) to afford N-(4-amino-1H-pyrazolo[4,3-c]pyridin-7-yl)-N′-benzyl-N′-[[3-(trifluoromethyl)-2-pyridyl]methyl]oxamide (63.6 mg, 123.39 μmol, 43.53% yield, HCOOH) as a yellow solid.

¹H NMR (600 MHz, DMSO-d₆) δ 4.51-4.76 (m, 2H), 4.96-5.25 (m, 2H), 6.93 (s, 2H), 7.27-7.41 (m, 5H), 7.46-7.58 (m, 1H), 7.59-7.75 (m, 1H), 8.07-8.12 (m, 1H), 8.18-8.26 (m, 1H), 8.77-8.88 (m, 1H), 10.36-10.67 (m, 1H), 12.92 (br s, 1H).

LCMS (ESI): [M+H]⁺m/z: calcd 470.17; found 470.2; Rt=2.948 min.

### Example 101. Synthesis of N-(4-amino-2H-pyrazolo[4,3-c]pyridin-7-yl)-N′-benzyl-N′-[(3-chloro-2-pyridyl)methyl]oxamid (Compound 120)

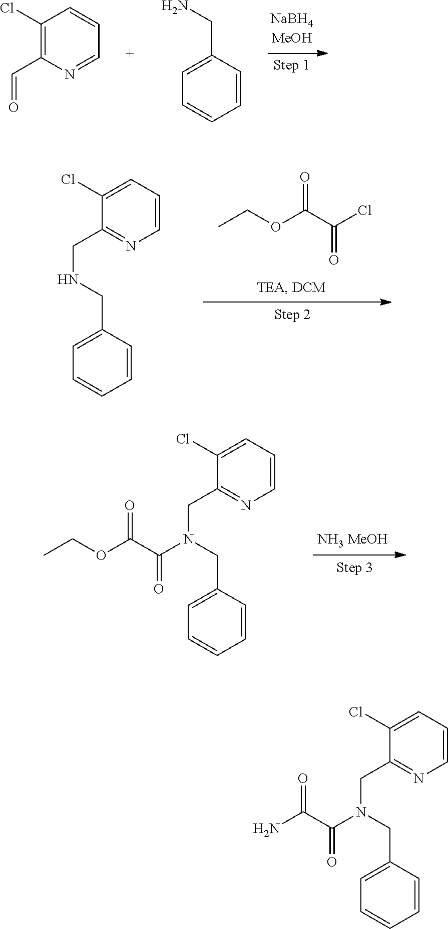

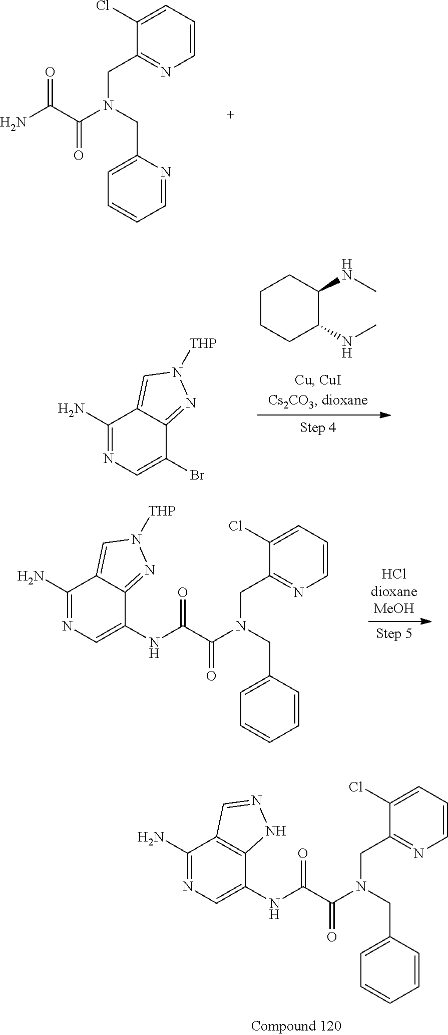

### Step 1: N-[(3-chloro-2-pyridyl)methyl]-1-phenyl-methanamine

Phenylmethanamine (1 g, 9.33 mmol) was added to a stirred solution of 3-chloropyridine-2-carbaldehyde (1.32 g, 9.33 mmol) in Methanol (50 mL) and was stirred at 20° C. for 10 hr. Then Sodium Borohydride (353.05 mg, 9.33 mmol, 328.72 μL) was added, the reaction mixture was stirred for 2 hr additional. The reaction mixture was evaporated in vacuo. The residue was dissolved in DCM (50 mL) and washed with water (2×15 mL). The organic layer was dried over anhydrous sodium sulfate and filtered. DCM was evaporated under reduce pressure to give N-[(3-chloro-2-pyridyl)methyl]-1-phenyl-methanamine (1.6 g, 6.88 mmol, 73.67% yield) as a yellow oil.

LCMS (ESI): [M+H]⁺m/z: calcd 233.09; found 233.0; Rt=0.862 min.

### Step 2: ethyl 2-[benzyl-[(3-chloro-2-pyridyl)methyl]amino]-2-oxo-acetate

N-[(3-chloro-2-pyridyl)methyl]-1-phenyl-methanamine (1.6 g, 6.88 mmol) and TEA (695.74 mg, 6.88 mmol, 958.32 μL) were dissolved in DCM (30 mL), cooled with ice-water bath and then ethyl 2-chloro-2-oxo-acetate (938.74 mg, 6.88 mmol, 768.20 μL) in 20 mL of DCM was added dropwise in 10 min. The solution was stirred overnight. The reaction mixture was washed with an aqueous solution of NaHCO₃, dried over anhydrous sodium sulfate and evaporated to give ethyl 2-[benzyl-[(3-chloro-2-pyridyl)methyl]amino]-2-oxo-acetate (2.1 g, 6.31 mmol, 91.78% yield) as a yellow oil.

LCMS (ESI): [M+H]⁺m/z: calcd 333.1; found 333.2; Rt=1.264 min.

### Step 3: N′-benzyl-N′-[(3-chloro-2-pyridyl)methyl]oxamide

Ethyl 2-[benzyl-[(3-chloro-2-pyridyl)methyl]amino]-2-oxo-acetate (2.1 g, 6.31 mmol) was dissolved in NH3/methanol (30 mL) solution (10% by weight) and stirred overnight, then concentrated in vacuo to give N′-benzyl-N′-[(3-chloro-2-pyridyl)methyl]oxamide (1.63 g, 5.37 mmol, 85.04% yield) as a yellow oil.

LCMS (ESI): [M+H]⁺m/z: calcd 304.09; found 304.2; Rt=1.161 min.

### Step 4: N-(4-amino-2-tetrahydropyran-2-yl-pyrazolo[4,3-c]pyridin-7-yl)-N′-benzyl-N′-[(3-chloro-2-pyridyl)methyl]oxamide

7-bromo-2-tetrahydropyran-2-yl-pyrazolo[4,3-c]pyridin-4-amine (293.49 mg, 987.68 μmol), N′-benzyl-N′-[(3-chloro-2-pyridyl)methyl]oxamide (0.2 g, 658.45 μmol), Copper (I) iodide (37.62 mg, 197.54 μmol, 6.69 μL), Cesium carbonate (429.07 mg, 1.32 mmol) and

(1R,2R)—N1,N2-dimethylcyclohexane-1,2-diamine (42.15 mg, 296.30 μmol) were mixed in Dioxane (4 mL) under argon, and then stirred overnight at 95° C. for 12 hr in vial. The reaction mixture was filtered, then evaporated. The residue was dissolved in 15 mL of DCM, washed with an aqueous ammonium solution, dried over anhydrous sodium sulfate and evaporated to give crude product which was purified by HPLC (Device (Mobile Phase, Column): SYSTEM 0-2-10 min 33-40-90% H₂O/MeOH/0.1% NH₄OH flow 30 mL/min ((loading pump 4 mL MeOH) column: Chromatorex SMB100-5T C18 100×19 mm, 5 microM) to afford N-(4-amino-2-tetrahydropyran-2-yl-pyrazolo[4,3-c]pyridin-7-yl)-N′-benzyl-N′-[(3-chloro-2-pyridyl)methyl]oxamide (0.028 g, 53.85 μmol, 8.18% yield) as a yellow gum.

LCMS (ESI): [M+H]⁺m/z: calcd 520.21; found 520.2; Rt=0.860 min.

### Step 5: The synthesis of N-(4-amino-2H-pyrazolo[4,3-c]pyridin-7-yl)-N′-benzyl-N′-[(3-chloro-2-pyridyl)methyl]oxamid

N-(4-amino-2-tetrahydropyran-2-yl-pyrazolo[4,3-c]pyridin-7-yl)-N′-benzyl-N′-[(3-chloro-2-pyridyl)methyl]oxamide (340.38 mg, 654.59 μmol) was dissolved in MeOH (1 mL) and dioxane/HCl (13.09 mmol, 1 mL) was added thereto. Then it was stirred at rt for 2 hr. The reaction mixture was evaporated. The crude product was purified by HPLC (Device (Mobile Phase, Column): SYSTEM 0-2-10 min 0-55% H₂O/ACN/0.1% FA, flow 30 mL/min (loading pump 4 mL water), target mass 435 column: Chromatorex SMB100-5T C18 100×19 mm, 5 microM) to afford N-(4-amino-2H-pyrazolo[4,3-c]pyridin-7-yl)-N′-benzyl-N′-[(3-chloro-2-pyridyl)methyl]oxamide (13.6 mg, 0.031 mmol, 4.77% yield) as a yellow solid ¹H NMR (600 MHz, DMSO-d₆) δ 4.58-4.74 (m, 2H), 4.94-5.16 (m, 2H), 6.57-6.68 (m, 2H), 7.24-7.29 (m, 1H), 7.29-7.36 (m, 4H), 7.36-7.38 (m, 1H), 7.53-7.69 (m, 1H), 7.80-7.93 (m, 1H), 8.14-8.19 (m, 1H), 8.47-8.56 (m, 1H), 10.37-10.56 (m, 1H), 12.65-12.82 (m, 1H).

LCMS (ESI): [M+H]⁺m/z: calcd 436.14; found 436.2; Rt=2.789 min.

### Example 102. Synthesis of N-(4-amino-1H-pyrazolo[4,3-c]pyridin-7-yl)-N′-[(2-chlorophenyl)methyl]-N′-(2-pyridylmethyl)oxamide (Compound 142)

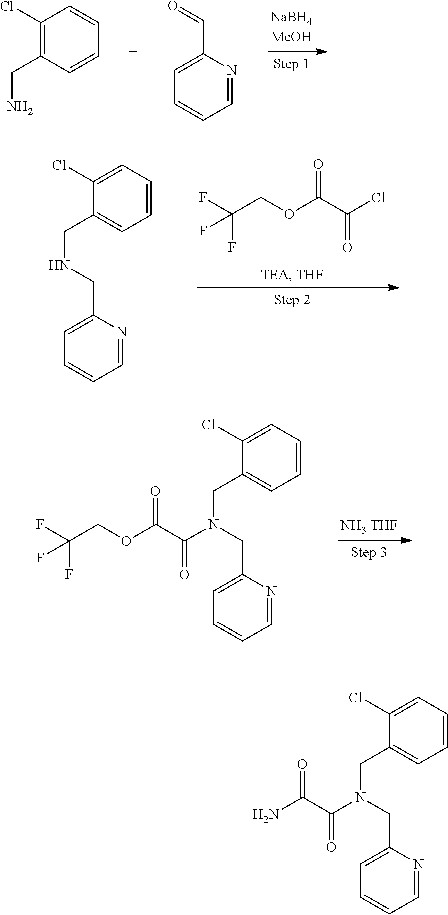

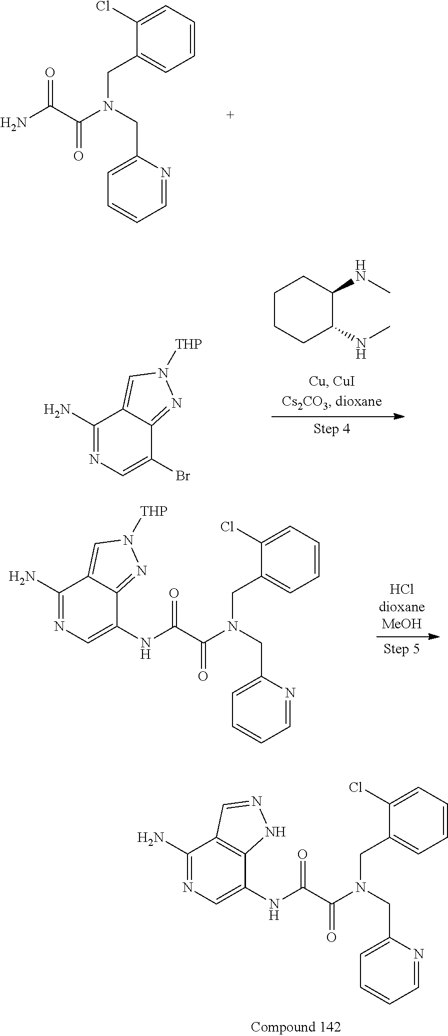

### Step 1:1-(2-chlorophenyl)-N-(2-pyridylmethyl)methanamine

A solution of pyridine-2-carbaldehyde (2 g, 18.67 mmol, 1.78 mL) and (2-chlorophenyl)methanamine (2.64 g, 18.67 mmol, 2.26 mL) in MeOH (39.93 mL) was stirred at 20° C. for 12 hr. To this solution, Sodium Borohydride (777.07 mg, 20.54 mmol, 723.53 μL) was added and the resulting mixture was stirred for 12 hr. The solvent was removed in vacuo, the residue was taken up with water (20 mL) and extracted with DCM (3×20 mL). The combined organic layer was washed with brine (10 mL), dried over anhydrous sodium sulfate and evaporated to obtain 1-(2-chlorophenyl)-N-(2-pyridylmethyl)methanamine (3.3 g, 14.18 mmol, 75.95% yield) as a yellow oil.

### Step 2:2,2,2-trifluoroethyl 2-[(2-chlorophenyl)methyl-(2-pyridylmethyl)amino]-2-oxo-acetate

To a solution of 1-(2-chlorophenyl)-N-(2-pyridylmethyl)methanamine (2 g, 8.59 mmol) and TEA (1.30 g, 12.89 mmol, 1.80 mL) in THF (25 mL) was added 2,2,2-trifluoroethyl 2-chloro-2-oxo-acetate (1.96 g, 10.31 mmol) dropwise at 0° C. under argon. The reaction mixture was then stirred for 12 hr at r.t., then evaporated in vacuo to give 2,2,2-trifluoroethyl 2-[(2-chlorophenyl)methyl-(2-pyridylmethyl)amino]-2-oxo-acetate (3 g, 7.76 mmol, 90.25% yield) as a yellow oil.

LCMS (ESI): [M+H]⁺m/z: calcd 387.07; found 387.0; Rt=3.913 min.

### Step 3: N′-[(2-chlorophenyl)methyl]-N′-(2-pyridylmethyl)oxamide

2,2,2-trifluoroethyl 2-[(2-chlorophenyl)methyl-(2-pyridylmethyl)amino]-2-oxo-acetate (3 g, 7.76 mmol) was dissolved in THF (20 mL) and was blow ammonium (2.64 g, 155.14 mmol). The resulting solution was stirred at 0° C. for 14 hr. The resulting mixture was evaporated in vacuo and residue was triturated with THF (2×20 mL), filtered and combined organic was evaporated in vacuity leave 1.8 g of crude product which was purification by column chromatography on silica gel using MTBE/CH₃OH gradient (10-100% MTBE) to afford N′-[(2-chlorophenyl)methyl]-N′-(2-pyridylmethyl)oxamide (0.6 g, 1.98 mmol, 25.47% yield) as a yellow oil.

LCMS (ESI): [M+H]⁺m/z: calcd 304.09; found 304.2; Rt=0.895 min.

### Step 4: N-(4-amino-1-tetrahydropyran-2-yl-pyrazolo[4,3-c]pyridin-7-yl)-N′-[(2-chlorophenyl)methyl]-N′-(2-pyridylmethyl)oxamide

7-bromo-1-tetrahydropyran-2-yl-pyrazolo[4,3-c]pyridin-4-amine (195.66 mg, 658.45 μmol), N′-[(2-chlorophenyl)methyl]-N′-(2-pyridylmethyl)oxamide (0.2 g, 658.45 μmol), Cu (2.09 mg, 32.92 μmol), CuI (125.40 mg, 658.45 μmol, 22.31 μL), cesium carbonate (321.80 mg, 987.68 μmol) and (1R,2R)—N1,N2-dimethylcyclohexane-1,2-diamine (112.39 mg, 790.14 μmol) were mixed in dioxane (6 mL), purged with Ar for 15 minutes and then heated in the sealed tube at 110° C. for 48 hr. Final mixture was filtered and dioxane was evaporated in vacuo. The crude product 0.4 g was purified by RP-HPLC (column: XBridge C18 5 μm 130A; 40-40-70% 0-1.5-5 min H₂O/CH₃CN/0.1% NH₄OH, flow: 30 mL/min) to give N-(4-amino-1-tetrahydropyran-2-yl-pyrazolo[4,3-c]pyridin-7-yl)-N′-[(2-chlorophenyl)methyl]-N′-(2-pyridylmethyl)oxamide (94.40 mg, 181.54 μmol, 27.57% yield) as a yellow gum.

LCMS (ESI): [M+H]⁺m/z: calcd 520.21; found 520.2; Rt=2.957 min.

### Step 5: The synthesis of N-(4-amino-1H-pyrazolo[4,3-c]pyridin-7-yl)-N′-[(2-chlorophenyl)methyl]-N′-(2-pyridylmethyl)oxamide

Hydrogen chloride, 4M in 1,4-dioxane, 99% (2.40 g, 65.82 mmol, 3 mL) was added to a solution of N-(4-amino-1-tetrahydropyran-2-yl-pyrazolo[4,3-c]pyridin-7-yl)-N′-[(2-chlorophenyl)methyl]-N′-(2-pyridylmethyl)oxamide (65.30 mg, 125.58 μmol) in MeOH (7.99 mL). The reaction mixture was stirred at 20° C. for 8 hr, then evaporated. The residue was purified by RP-HPLC (column: XBridge BEH18 SMB100-BT 100×19 mm; 35-35-60% 0-1.5-5 min H₂O/CH₃CN/0.1% NH₄OH, flow: 30 mL/min) to give N-(4-amino-1H-pyrazolo[4,3-c]pyridin-7-yl)-N′-[(2-chlorophenyl)methyl]-N′-(2-pyridylmethyl)oxamide (0.0316 g, 72.50 μmol, 57.73% yield) as a white solid.

¹H NMR (600 MHz, DMSO-d₆) δ 4.19-5.08 (m, 4H), 6.52-6.75 (m, 2H), 6.92-7.35 (m, 4H), 7.35-7.61 (m, 3H), 7.61-7.67 (m, 1H), 7.73-7.82 (m, 1H), 8.11-8.21 (m, 1H), 8.46-8.52 (m, 1H), 9.64-13.54 (m, 1H).

LCMS (ESI): [M+H]⁺m/z: calcd 436.14; found 436.2; Rt=2.393 min.

### Example 103. Synthesis of N-(4-amino-2HI-pyrazolo[4,3-c]pyridin-7-yl)-N′-benzyl-N′-[(5-fluoro-3-methyl-2-pyridyl)methyl]oxamide (Compound 116)

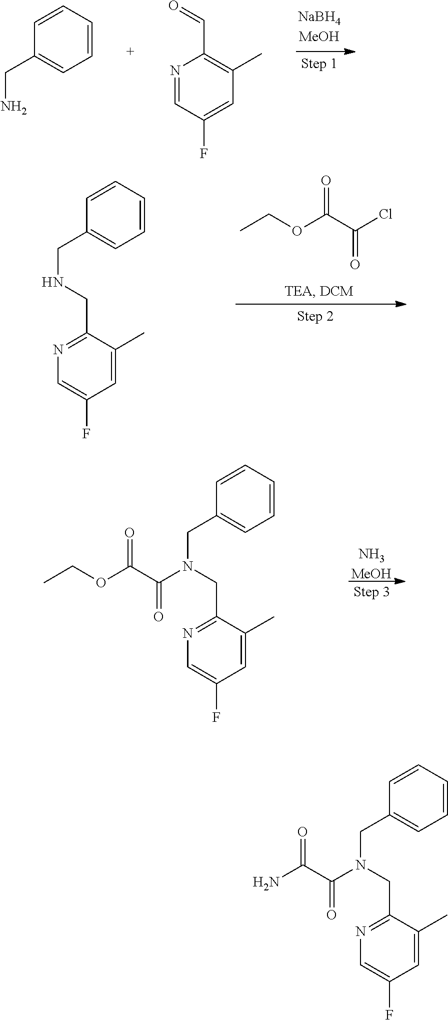

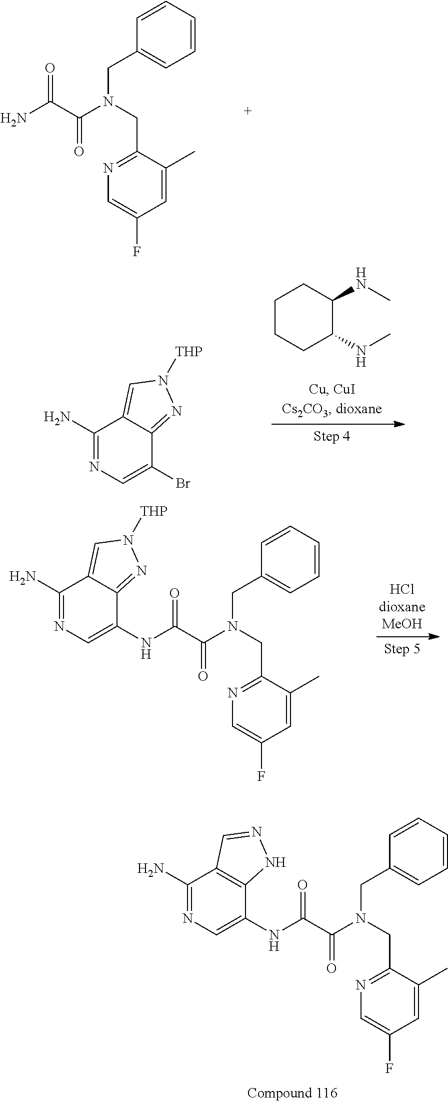

### Step 1: N-[(5-fluoro-3-methyl-2-pyridyl)methyl]-1-phenyl-methanamine

Phenylmethanamine (462.11 mg, 4.31 mmol) was added to a stirred solution of 5-fluoro-3-methyl-pyridine-2-carbaldehyde (0.6 g, 4.31 mmol) in Methanol (20 mL) and was stirred at 20° C. for 10 hr. Then Sodium Borohydride (163.15 mg, 4.31 mmol, 151.91 μL) was added, the reaction mixture was stirred for 2 hr additional. The reaction mixture was evaporated in vacuo. The residue was dissolved in DCM (50 mL) and washed with water (2×15 mL). The organic layer was dried over anhydrous sodium sulfate and filtered. DCM was evaporated under reduce pressure to give N-[(5-fluoro-3-methyl-2-pyridyl)methyl]-1-phenyl-methanamine (0.6 g, 2.61 mmol, 60.42% yield) as a yellow oil.

LCMS (ESI): [M+H]⁺m/z: calcd 231.13; found 231.2; Rt=0.894 min.

### Step 2: ethyl 2-[benzyl-[(5-fluoro-3-methyl-2-pyridyl)methyl]amino]-2-oxo-acetate

N-[(5-fluoro-3-methyl-2-pyridyl)methyl]-1-phenyl-methanamine (0.6 g, 2.61 mmol) and TEA (263.65 mg, 2.61 mmol, 363.16 μL) were dissolved in DCM (31.07 mL), cooled with ice-water bath and then ethyl 2-chloro-2-oxo-acetate (355.74 mg, 2.61 mmol, 291.11 μL) in 20 mL of DCM which was added dropwise in 10 min. The solution was stirred overnight. The reaction mixture was washed with an aqueous solution of NaHCO₃, dried over anhydrous sodium sulfate and evaporated to give ethyl 2-[benzyl-[(5-fluoro-3-methyl-2-pyridyl)methyl]amino]-2-oxo-acetate (1.1 g, 3.33 mmol, 127.80% yield) as a yellow oil.

LCMS (ESI): [M+H]⁺m/z: calcd 331.15; found 331.2; Rt=1.266 min.

### Step 3: N′-benzyl-N′-[(5-fluoro-3-methyl-2-pyridyl)methyl]oxamide

Ethyl 2-[benzyl-[(5-fluoro-3-methyl-2-pyridyl)methyl]amino]-2-oxo-acetate (1.1 g, 3.33 mmol) was dissolved in NH3/methanol (30 mL) solution (10% by weight) and stirred overnight, then concentrated in vacuo to give N′-benzyl-N′-[(5-fluoro-3-methyl-2-pyridyl)methyl]oxamide (0.75 g, 2.49 mmol, 74.75% yield) as a yellow oil.

LCMS (ESI): [M+H]⁺m/z: calcd 302.13; found 302.0; Rt=1.137 min.

### Step 4: N-(4-amino-2-tetrahydropyran-2-yl-pyrazolo[4,3-c]pyridin-7-yl)-N′-benzyl-N′-[(5-fluoro-3-methyl-2-pyridyl)methyl]oxamide

7-bromo-2-tetrahydropyran-2-yl-pyrazolo[4,3-c]pyridin-4-amine (293.49 mg, 987.68 μmol), N′-benzyl-N′-[(5-fluoro-3-methyl-2-pyridyl)methyl]oxamide (198.40 mg, 658.45 μmol), Copper (I) iodide (37.62 mg, 197.54 μmol, 6.69 μL), Cesium carbonate (429.07 mg, 1.32 mmol) and (1R,2R)—N1,N2-dimethylcyclohexane-1,2-diamine (42.15 mg, 296.30 μmol) were mixed in Dioxane (4 mL) under argon, and then stirred overnight at 95° C. for 12 hr in vial. The reaction mixture was filtered, then evaporated. The residue was dissolved in 15 mL of DCM, washed with an aqueous ammonium solution, dried over anhydrous sodium sulfate, and evaporated. The residue was subjected to HPLC (Device (Mobile Phase, Column): SYSTEM 0-2-10 min 33-40-90% H₂O/MeOH/0.1% NH₄OH flow 30 mL/min ((loading pump 4 mL MeOH), column: Chromatorex SMB100-5T C18 100×19 mm, 5 microM) to give N-(4-amino-2-tetrahydropyran-2-yl-pyrazolo[4,3-c]pyridin-7-yl)-N′-benzyl-N′-[(5-fluoro-3-methyl-2-pyridyl)methyl]oxamide (0.0108 g, 20.87 μmol, 3.17% yield) as a light-yellow gum.

LCMS (ESI): [M+H]⁺m/z: calcd 518.26; found 518.2; Rt=0.867 min.

### Step 5: The synthesis of N-(4-amino-2H-pyrazolo[4,3-c]pyridin-7-yl)-N′-benzyl-N′-[(5-fluoro-3-methyl-2-pyridyl)methyl]oxamide

N-(4-amino-2-tetrahydropyran-2-yl-pyrazolo[4,3-c]pyridin-7-yl)-N′-benzyl-N′-[(5-fluoro-3-methyl-2-pyridyl)methyl]oxamide (338.79 mg, 654.59 μmol) was dissolved in MeOH (1 mL) and dioxane/HCl (13.09 mmol, 1 mL) was added thereto. Then it was stirred at rt for 2 hr. The reaction mixture was evaporated. The crude product was purified by HPLC (Device (Mobile Phase, Column): SYSTEM 0-2-10 min 0-55% H₂O/ACN/0.1% FA, flow 30 mL/min (loading pump 4 mL water), target mass 433, column: Chromatorex SMB100-5T C18 100×19 mm, 5 microM) to afford N-(4-amino-2H-pyrazolo[4,3-c]pyridin-7-yl)-N′-benzyl-N′-[(5-fluoro-3-methyl-2-pyridyl)methyl]oxamide as a yellow solid.

¹H NMR (600 MHz, DMSO-d₆) δ 2.13-2.29 (m, 3H), 4.49-4.67 (m, 2H), 4.86-4.97 (m, 2H), 6.58-6.68 (m, 2H), 7.25-7.35 (m, 5H), 7.44-7.57 (m, 1H), 7.57-7.69 (m, 1H), 8.09-8.18 (m, 1H), 8.32-8.39 (m, 1H), 8.54-8.84 (m, 1H), 10.34-10.54 (m, 1H), 12.64-12.78 (m, 1H). (salt?)

LCMS (ESI): [M+H]⁺m/z: calcd 434.19; found 434.2; Rt=2.776 min.

### Example 104. Synthesis of N-(4-amino-2H-pyrazolo[4,3-c]pyridin-7-yl)-N′-benzyl-N′—[(2,3-dimethylphenyl)methyl]oxamide (Compound 130)

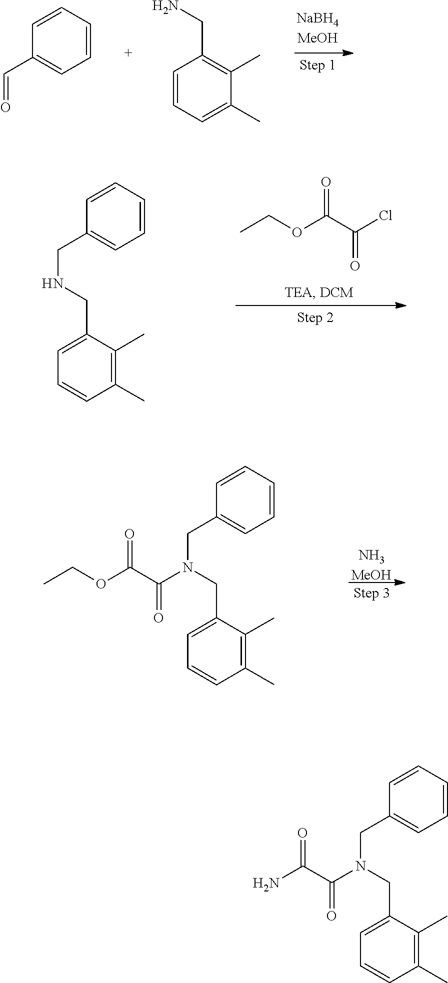

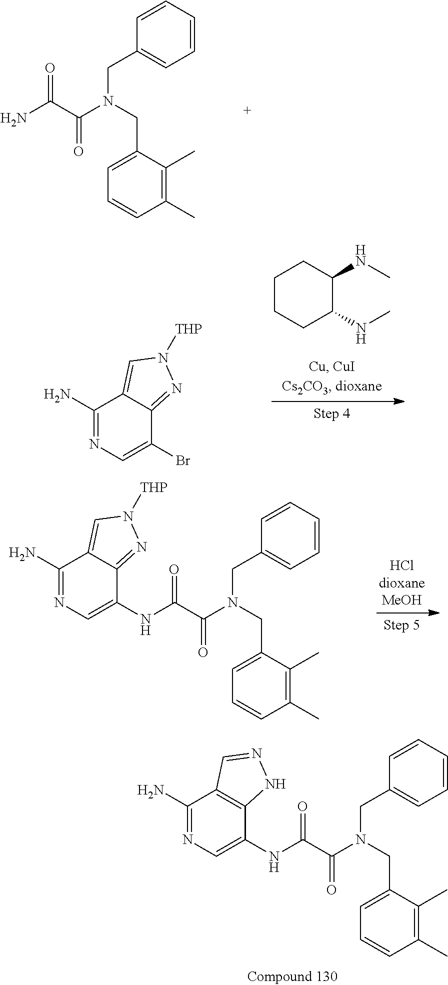

### Step 1: N-[(2,3-dimethylphenyl)methyl]-1-phenyl-methanamine

Benzaldehyde (1 g, 9.42 mmol) was added to a stirred solution of (2,3-dimethylphenyl)methanamine (1.27 g, 9.42 mmol) in Methanol (50 mL) and was stirred at 20° C. for 10 hr. Then Sodium Borohydride (356.48 mg, 9.42 mmol, 331.92 μL) was added, the reaction mixture was stirred for 2 hr additional. The reaction mixture was evaporated in vacuo. The residue was dissolved in DCM (50 mL) and washed with water (2×15 mL). The organic layer was dried over anhydrous sodium sulfate and filtered. DCM was evaporated under reduce pressure to give N-[(2,3-dimethylphenyl)methyl]-1-phenyl-methanamine (1.8 g, 7.99 mmol, 84.77% yield) as a yellow oil.

LCMS (ESI): [M+H]⁺m/z: calcd 226.16; found 226.2; Rt=0.802 min.

### Step 2: ethyl 2-[benzyl-[(2,3-dimethylphenyl)methyl]amino]-2-oxo-acetate

N-[(2,3-dimethylphenyl)methyl]-1-phenyl-methanamine (1.8 g, 7.99 mmol) and TEA (808.34 mg, 7.99 mmol, 1.11 mL) were dissolved in DCM (50 mL), cooled with ice-water bath and then ethyl 2-chloro-2-oxo-acetate (1.09 g, 7.99 mmol, 892.54 μL) in 20 mL of DCM was added dropwise in 10 min. The solution was stirred overnight. The reaction mixture was washed with an aqueous solution of NaHCO₃, dried over anhydrous sodium sulfate and evaporated to give ethyl 2-[benzyl-[(2,3-dimethylphenyl)methyl]amino]-2-oxo-acetate (2.3 g, 7.07 mmol, 88.48% yield) as a yellow oil.

LCMS (ESI): [M+H]⁺m/z: calcd 326.18; found 326.2; Rt=1.298 min.

### Step 3: N′-benzyl-N′—[(2,3-dimethylphenyl)methyl]oxamide

Ethyl 2-[benzyl-[(2,3-dimethylphenyl)methyl]amino]-2-oxo-acetate (2.3 g, 7.07 mmol) was dissolved in NH3/methanol (50 mL) solution (10% by weight) and stirred overnight, then concentrated in vacuo to give N′-benzyl-N′—[(2,3-dimethylphenyl)methyl]oxamide (1.81 g, 6.11 mmol, 86.41% yield) as a yellow oil.

### Step 4: N-(4-amino-2-tetrahydropyran-2-yl-pyrazolo[4,3-c]pyridin-7-yl)-N′-benzyl-N′—[(2,3-dimethylphenyl)methyl]oxamide

7-bromo-2-tetrahydropyran-2-yl-pyrazolo[4,3-c]pyridin-4-amine (0.3 g, 1.01 mmol), N′-benzyl-N′—[(2,3-dimethylphenyl)methyl]oxamide (199.47 mg, 673.06 μmol), Copper (I) iodide (38.46 mg, 201.92 μmol, 6.84 μL), Cesium carbonate (438.59 mg, 1.35 mmol) and (1R,2R)—N1,N2-dimethylcyclohexane-1,2-diamine (43.08 mg, 302.88 μmol) were mixed in Dioxane (4 mL) under argon, and then stirred overnight at 95° C. for 12 hr in vial. The reaction mixture was filtered, then evaporated. The residue was dissolved in 15 mL of DCM washed with an aqueous ammonium solution, dried over anhydrous sodium sulfate, and evaporated to give crude product. The crude product was purified by HPLC (Device (Mobile Phase, Column): SYSTEM 0-2-10 min 48-55-90% H₂O/MeOH/0.1% NH₄OH, flow 30 mL/min ((loading pump 4 mL MeOH/0.1% NH₄OH), target mass 512, column: XBridge BEH C18 100×19 mm, 5 microM) to afford N-(4-amino-2-tetrahydropyran-2-yl-pyrazolo[4,3-c]pyridin-7-yl)-N′-benzyl-N′—[(2,3-dimethylphenyl)methyl]oxamide (0.058 g, 113.15 μmol, 16.81% yield).

LCMS (ESI): [M+H]⁺m/z: calcd 513.3; found 513.4; Rt=1.189 min.

### Step 5: The synthesis of N-(4-amino-2H-pyrazolo[4,3-c]pyridin-7-yl)-N′-benzyl-N′—[(2,3-dimethylphenyl)methyl]oxamide

N-(4-amino-2-tetrahydropyran-2-yl-pyrazolo[4,3-c]pyridin-7-yl)-N′-benzyl-N′—[(2,3-dimethylphenyl)methyl]oxamide (0.058 g, 113.15 μmol) was dissolved in MeOH (1 mL) and dioxane/HCl (2.26 mmol, 1 mL) was added thereto. Then it was stirred at rt for 2 hr. The reaction mixture was evaporated. The crude product was purified by HPLC (Device (Mobile Phase, Column): SYSTEM 0-2-10 min 0-0-55% H₂O/ACN/0.1% FA, flow 30 mL/min (loading pump 4 mL ACN), target mass 428, column: Chromatorex SMB100-5T C18 100×19 mm, 5 microM) to afford N-(4-amino-2H-pyrazolo[4,3-c]pyridin-7-yl)-N′-benzyl-N′—[(2,3-dimethylphenyl)methyl]oxamide (0.0147 g, 34.31 μmol, 30.32% yield) as a beige solid.

¹H NMR (600 MHz, DMSO-d₆) § 1.59-2.26 (m, 6H), 4.05-4.81 (m, 5H), 5.62-6.75 (m, 2H), 6.89-7.38 (m, 8H), 7.53-7.68 (m, 1H), 8.15-8.84 (m, 1H), 9.73-10.73 (m, 1H), 12.47-13.56 (m, 1H).

LCMS (ESI): [M+H]⁺m/z: calcd 429.23; found 429.0; Rt=2.789 min.

### Example 105. Synthesis of N-(4-amino-1H-pyrazolo[4,3-c]pyridin-7-yl)-N′-benzyl-N′-(1-naphthylmethyl)oxamide (Compound 122)

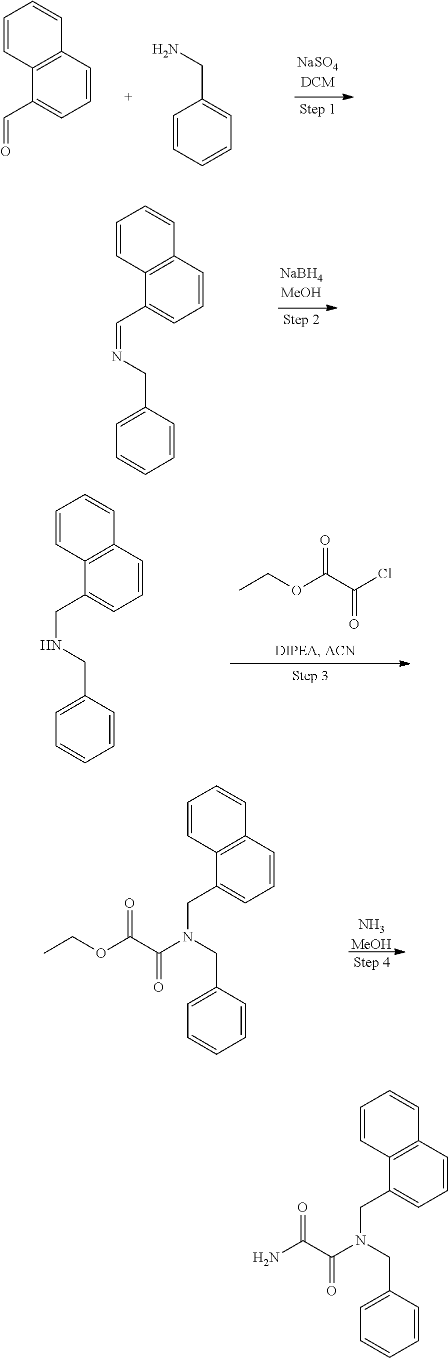

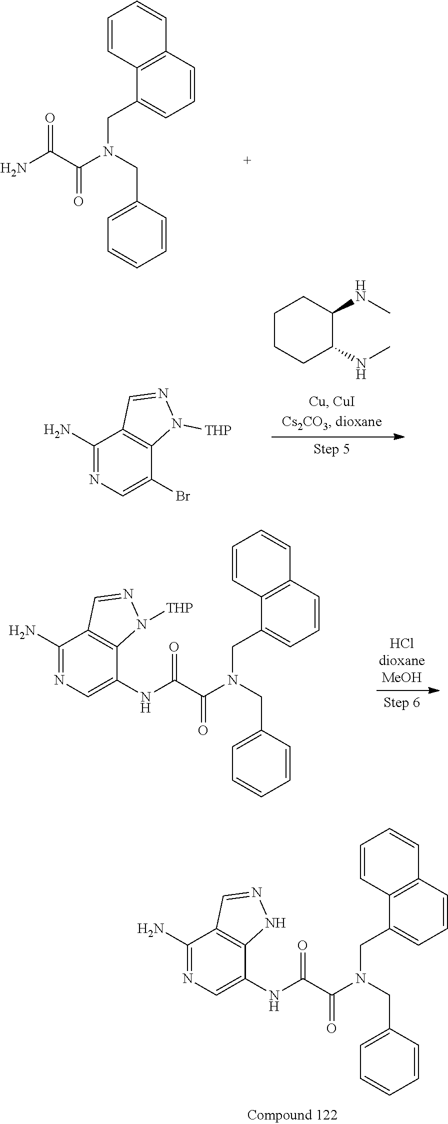

### Step 1: (E)-N-benzyl-1-(1-naphthyl) methanimine

Naphthalene-1-carbaldehyde (3 g, 19.21 mmol, 2.61 mL), phenylmethanamine (2.06 g, 19.21 mmol) and Sodium sulfate, anhydrous (10 g, 70.40 mmol, 3.73 mL) was mixed in DCM (100 mL) and stirred at RT overnight. Upon completion, the reaction mixture was filtered and filtrate was concentrated under reduced pressure to afford (E)-N-benzyl-1-(1-naphthyl) methanimine (4.6 g, crude) which was directly used in the next step without purification and analytical data collection.

### Step 2: N-(1-naphthylmethyl)-1-phenyl-methanamine

(E)-N-benzyl-1-(1-naphthyl) methanimine (4.6 g, 18.75 mmol) was dissolved in methanol (100. mL) and Sodium Borohydride (922.23 mg, 24.38 mmol, 858.69 μL) was added portionwise to a mixture with stirring. The resulting solution was stirred at RT overnight and concentrated in vacuo. Residue was taken up in DCM, washed with NaHCO₃ solution and concentrated under reduced pressure to afford N-(1-naphthylmethyl)-1-phenyl-methanamine (3.5 g, crude).

LCMS (ESI): [M+H]⁺m/z: calcd 248.15; found 248.2; Rt=0.883 min.

### Step 3: ethyl 2-[benzyl(1-naphthylmethyl)amino]-2-oxo-acetate

N-(1-naphthylmethyl)-1-phenyl-methanamine (1.5 g, 6.06 mmol) was dissolved in DCM (40 mL) and the solution was cooled with ice bath. DIPEA (1.18 g, 9.10 mmol, 1.58 mL) was added to the solution dropwise and reaction mixture was stirred at RT overnight. Upon completion, obtained DCM solution was washed with water several times, dried over anhydrous sodium sulfate and concentrated in vacuo to afford ethyl 2-[benzyl(1-naphthylmethyl)amino]-2-oxo-acetate (2 g, crude).

LCMS (ESI): [M+H]⁺m/z: calcd 348.16; found 348.2; Rt=1.346 min.

### Step 4: N′-benzyl-N′-(1-naphthylmethyl)oxamide

Ethyl 2-[benzyl(1-naphthylmethyl)amino]-2-oxo-acetate (2 g, 5.76 mmol) was added to saturated methanol/ammonia solution and the mixture was stirred at RT overnight. Upon completion, the reaction mixture was concentrated in vacuo to afford N′-benzyl-N′-(1-naphthylmethyl)oxamide (1.68 g, crude).

### Step 5: N-(4-amino-1-tetrahydropyran-2-yl-pyrazolo[4,3-c]pyridin-7-yl)-N′-benzyl-N′-(1-naphthylmethyl)oxamide

N′-benzyl-N′-(1-naphthylmethyl)oxamide (0.3 g, 942.30 μmol), 7-bromo-1-tetrahydropyran-2-yl-pyrazolo[4,3-c]pyridin-4-amine (308.01 mg, 1.04 mmol), copper (31.78 mg, 500.13 μmol), Copper (I) iodide (179.46 mg, 942.30 μmol, 31.93 μL), (1S,2S)-N,N′-Bis -methyl-1,2-cyclohexane-diamine (134.03 mg, 942.30 μmol, 148.60 μL) were mixed in dioxane (15 mL). The reaction mixture was stirred under Ar atmosphere at 100° C. for 16 hr. Upon completion, the mixture was diluted with water and extracted with DCM. Organic layer was separated, dried over anhydrous sodium sulfate and concentrated under reduced pressure to afford N-(4-amino-1-tetrahydropyran-2-yl-pyrazolo[4,3-c]pyridin-7-yl)-N′-benzyl-N′-(1-naphthylmethyl)oxamide (0.37 g, 692.10 μmol, 73.45% yield) which was used in the next step without purification.

LCMS (ESI): [M+H]⁺m/z: calcd 535.25; found 535.2; Rt=1.321 min.

### Step 6: The synthesis of N-(4-amino-1H-pyrazolo[4,3-c]pyridin-7-yl)-N′-benzyl-N′-(1-naphthylmethyl)oxamide

N-(4-amino-1-tetrahydropyran-2-yl-pyrazolo[4,3-c]pyridin-7-yl)-N′-benzyl-N′-(1-naphthylmethyl)oxamide (0.37 g, 692.10 μmol) was added to a mixture of HCl (4.0 M in 1,4-dioxane)(25.23 mg, 692.10 μmol, 2 mL) and methanol (0.5 mL) and stirred at 25° C. for 2 hr. Upon completion, the reaction mixture was concentrated in vacuo and the residue was submitted to HPLC (0-2-10 min 38-45-65% MeOH/H₂O+NH₄OH flow 30 mL/min (loading pump 4 mL MeOH), target MI 451, column: Chromatorex SMB100-5T 100×19, 5 microM), then repurified by HPLC (0-2-10 min 33-40-65% MeOH/H₂O+FA 30 mL/min (loading pump 4 mL MeOH), target MI 451, column: Chromatorex C18 SMB100-5T 100×19, 5microM) to afford N-(4-amino-1H-pyrazolo[4,3-c]pyridin-7-yl)-N′-benzyl-N′-(1-naphthylmethyl)oxamide (0.043 g, 86.60 μmol, 12.51% yield, HCOOH) as a yellow solid.

¹H NMR (600 MHz, DMSO-d₆) δ 4.32-4.74 (m, 2H), 4.74-5.31 (m, 2H), 5.91-6.74 (m, 2H), 6.99-7.26 (m, 1H), 7.26-7.34 (m, 4H), 7.38-7.53 (m, 2H), 7.53-7.56 (m, 1H), 7.56-7.67 (m, 1H), 7.67-7.88 (m, 1H), 7.88-8.06 (m, 2H), 8.13-8.85 (m, 2H), 9.79-10.74 (m, 1H), 12.54-13.69 (m, 1H).

LCMS (ESI): [M+H]⁺m/z: calcd 451.21; found 451.4; Rt=1.691 min.

### Example 106. Synthesis of N-(4-amino-2H-pyrazolo[4,3-c]pyridin-7-yl)-N′-methyl-N′-[(2-phenylphenyl)methyl]oxamide (Compound 164)

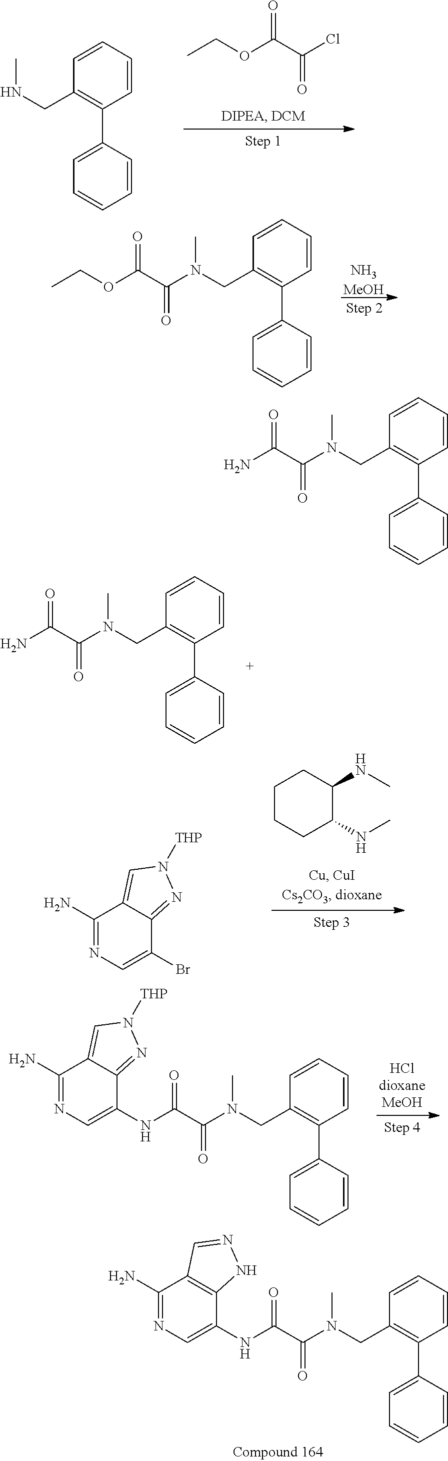

### Step 1: ethyl 2-[methyl-[(2-phenylphenyl)methyl]amino]-2-oxo-acetate

Ethyl 2-chloro-2-oxo-acetate (1.52 g, 11.15 mmol, 1.25 mL) was added dropwise to an ice bath cooled stirred solution of N-methyl-1-(2-phenylphenyl)methanamine (2 g, 10.14 mmol) and DIPEA (1.70 g, 13.18 mmol, 2.30 mL) in DCM (50 mL). The reaction mixture was stirred overnight. Upon completion, the reaction mixture was washed with water. The organic layer was separated, dried over anhydrous sodium sulfate and concentrated under reduced pressure to afford ethyl 2-[methyl-[(2-phenylphenyl)methyl]amino]-2-oxo-acetate (3.1 g, crude).

LCMS (ESI): [M+H]⁺m/z: calcd 298.15; found 298.2; Rt=1.315 min.

### Step 2: N′-methyl-N′-[(2-phenylphenyl)methyl]oxamide

Ethyl 2-[methyl-[(2-phenylphenyl)methyl]amino]-2-oxo-acetate (3.1 g, 10.43 mmol) was dissolved in methanol saturated with ammonia. The reaction mixture was stirred overnight and concentrated under reduced pressure to afford N′-methyl-N′-[(2-phenylphenyl)methyl]oxamide (2.7 g, crude).

LCMS (ESI): [M−H]⁻ m/z: calcd 267.11; found 267.2; Rt=1.218 min.

### Step 3: N-(4-amino-2-tetrahydropyran-2-yl-pyrazolo[4,3-c]pyridin-7-yl)-N′-methyl-N′-[(2-phenylphenyl)methyl]oxamide

N′-methyl-N′-[(2-phenylphenyl)methyl]oxamide (0.3 g, 1.12 mmol), 7-bromo-2-tetrahydropyran-2-yl-pyrazolo[4,3-c]pyridin-4-amine (332.25 mg, 1.12 mmol), copper (42.45 mg, 668.01 μmol), Copper (I) iodide (212.94 mg, 1.12 mmol, 37.89 μL), (1S,2S)-N,N′-Bis -methyl-1,2-cyclohexane-diamine (159.04 mg, 1.12 mmol, 176.32 μL) were mixed in dioxane (12.07 mL). The reaction mixture was stirred under Ar atmosphere at 100° C. for 48 hr. Upon completion, the mixture was diluted with water and extracted with DCM. Organic layer was washed with aq. ammonia, separated, dried over anhydrous sodium sulfate and concentrated under reduced pressure to afford N-(4-amino-2-tetrahydropyran-2-yl-pyrazolo[4,3-c]pyridin-7-yl)-N′-methyl-N′-[(2-phenylphenyl)methyl]oxamide (0.41 g, crude) which was used in the next step without purification.

LCMS (ESI): [M+H]⁺m/z: calcd 485.23; found 485.2; Rt=1.241 min.

### Step 4: The synthesis of N-(4-amino-2H-pyrazolo[4,3-c]pyridin-7-yl)-N′-methyl-N′-[(2-phenylphenyl)methyl]oxamide

N-(4-amino-2-tetrahydropyran-2-yl-pyrazolo[4,3-c]pyridin-7-yl)-N′-methyl-N′-[(2-phenylphenyl)methyl]oxamide (0.41 g, 846.15 μmol) was dissolved in HCl/dioxane solution and stirred at 20° C. for 4 hr. Upon completion the reaction mixture was concentrated under reduced pressure and the residue was submitted to HPLC (0-2-10 min 0-0-55% H₂O/ACN/0.1FA, flow 30 mL/min (loading pump 4 mL ACN)), then repurified by HPLC (0-2-10 min 2-30-55% H₂O/MeOH/0.1NH4OH, flow 30 mL/min (loading pump 4 mL MeOH)) to afford N-(4-amino-2H-pyrazolo[4,3-c]pyridin-7-yl)-N′-methyl-N′-[(2-phenylphenyl)methyl]oxamide (0.03 g, 67.20 μmol, 7.94% yield, HCOOH) as a yellow solid.

¹H NMR (600 MHz, DMSO-d₆) § 2.66-2.97 (m, 3H), 4.49-4.80 (m, 2H), 6.63-6.93 (m, 2H), 7.21-7.28 (m, 1H), 7.29-7.32 (m, 1H), 7.33-7.39 (m, 3H), 7.39-7.43 (m, 2H), 7.46-7.70 (m, 2H), 8.11-8.20 (m, 2H), 9.62-10.46 (m, 1H), 12.50-13.49 (m, 1H).

LCMS (ESI): [M+H]⁺m/z: calcd 401.19; found 401.4; Rt=1.449 min.

### Example 107. Synthesis of N-(4-amino-1H-pyrazolo[4,3-c]pyridin-7-yl)-N′-[(5-fluoro-3-methyl-2-pyridyl)methyl]-N′-[(5-fluoro-2-pyridyl)methyl]oxamide (Compound 88)

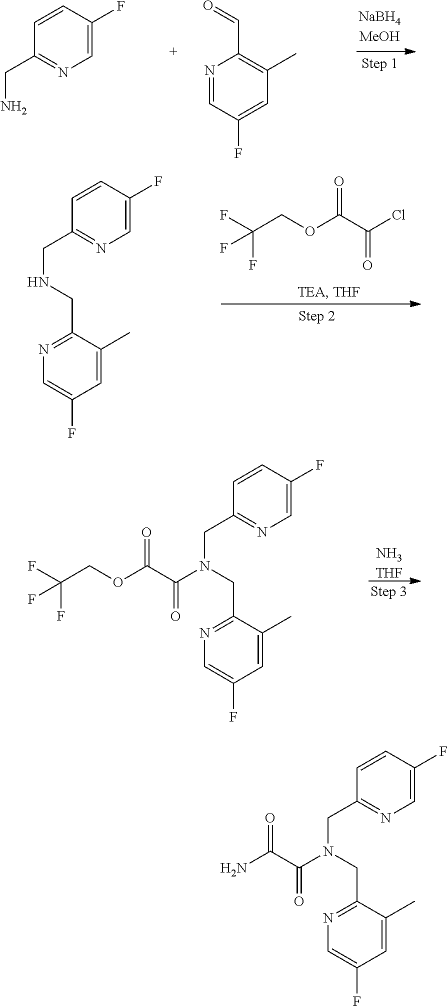

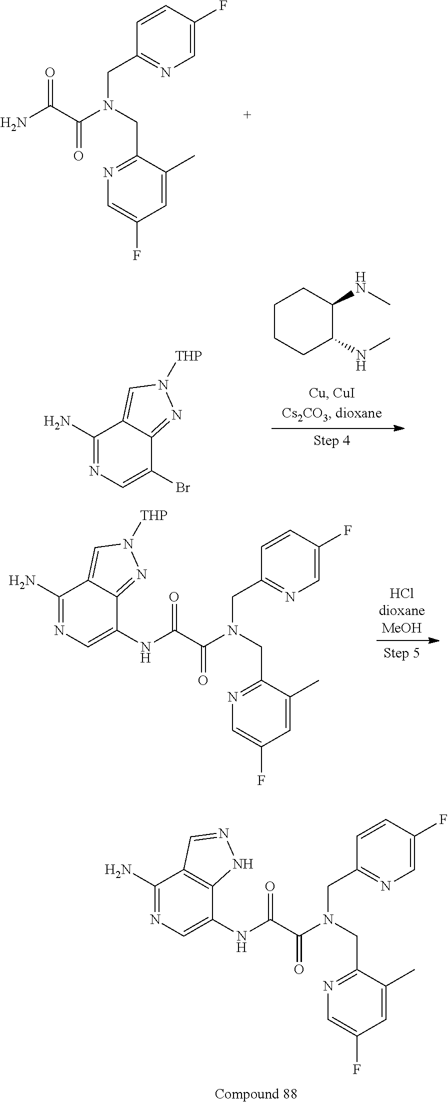

### Step 1: N-[(5-fluoro-3-methyl-2-pyridyl)methyl]-1-(5-fluoro-2-pyridyl)methanamine

A solution of (5-fluoro-2-pyridyl)methanamine (0.45 g, 3.57 mmol) and 5-fluoro-3-methyl-pyridine-2-carbaldehyde (496.36 mg, 3.57 mmol) in Methanol (50 mL) was stirred at 25° C. for 12 hr. To this solution, Sodium Borohydride (134.98 mg, 3.57 mmol, 125.68 μL) was added and the resulting mixture was stirred for 12 hr. This compound was combined with a separate batch before treatment. The solvent was removed in vacuo, the residue was taken up with water (20 mL) and extracted with DCM (3×25 mL). The combined organic layer was washed with brine (15 mL), dried over anhydrous sodium sulfate and evaporated to obtain N-[(5-fluoro-3-methyl-2-pyridyl)methyl]-1-(5-fluoro-2-pyridyl)methanamine (0.8 g, 3.21 mmol, 89.96% yield) as a yellow oil.

LCMS (ESI): [M+H]⁺m/z: calcd 250.12; found 250.2; Rt=0.760 min.

### Step 2:2,2,2-trifluoroethyl 2-[(5-fluoro-3-methyl-2-pyridyl)methyl-[(5-fluoro-2-pyridyl)methyl]amino]-2-oxo-acetate

To a solution of N-[(5-fluoro-3-methyl-2-pyridyl)methyl]-1-(5-fluoro-2-pyridyl)methanamine (0.5 g, 2.01 mmol) and Triethylamine (1.01 g, 10.03 mmol, 1.40 mL) in THF (30 mL), 2,2,2-trifluoroethyl 2-chloro-2-oxo-acetate (955.36 mg, 5.01 mmol) was added portionwise at 0° C. The resulting mixture was stirred for 3 hr. LCMS showed full conversion of SM. The reaction mixture was directly used for the next step.

LCMS (ESI): [M+H]⁺m/z: calcd 404.11; found 404.2; Rt=1.232 min.

### Step 3: N′-[(5-fluoro-3-methyl-2-pyridyl)methyl]-N′-[(5-fluoro-2-pyridyl)methyl]oxamide

Through a solution of 2,2,2-trifluoroethyl 2-[(5-fluoro-3-methyl-2-pyridyl)methyl-[(5-fluoro-2-pyridyl)methyl]amino]-2-oxo-acetate (0.8 g, 1.98 mmol) in THF (40 mL), ammonia was bubbled during 10 min at 0° C. The formed precipitate was filtered off, washed with THE (30 mL) and the solvent was evaporated in vacuo to give crude product (0.8 g), which was purified by HPLC (Device (Mobile Phase, Column): SYSTEM 25-25-75% 0-1-6 min H₂O/MeOH, flow: 30 mL/min (loading pump 4 mL/min MeOH) target mass 320 column: Chromatorex 18 SNB100-5T 100×19 mm 5 μm) to afford N′-[(5-fluoro-3-methyl-2-pyridyl)methyl]-N′-[(5-fluoro-2-pyridyl)methyl]oxamide (293 mg, 914.79 μmol, 46.12% yield) as a light-yellow solid.

LCMS (ESI): [M+H]⁺m/z: calcd 321.13; found 321.0; Rt=2.235 min.

### Step 4: N-(4-amino-2-tetrahydropyran-2-yl-pyrazolo[4,3-c]pyridin-7-yl)-N′-[(5-fluoro-3-methyl-2-pyridyl)methyl]-N′-[(5-fluoro-2-pyridyl)methyl]oxamide

A mixture of N′-[(5-fluoro-3-methyl-2-pyridyl)methyl]-N′-[(5-fluoro-2-pyridyl)methyl]oxamide (190 mg, 593.21 μmol), 7-bromo-2-tetrahydropyran-2-yl-pyrazolo[4,3-c]pyridin-4-amine (300 mg, 1.01 mmol), copper (1.92 mg, 30.25 μmol), Copper (I) iodide (100 mg, 525.07 μmol, 17.79 μL), cesium carbonate (309.25 mg, 949.13 μmol) and rac-(1R,2R)—N1,N2-dimethylcyclohexane-1,2-diamine (100 mg, 703.04 μmol) in 1,4-dioxane (6 mL) was stirred in a sealed vial under argon at 110° C. for 48 hr. The resulting mixture was cooled down, diluted with DMSO(1 mL) and submitted to reverse phase HPLC (column: XBridge C18 OBD 100×19 mm 5 μm; mobile phase: 35-50% 0-1-6 min H₂O/Acetonitrile; flow rate: 30 mL/min (loading pump 4 mL/min acetonitrile) to afford N-(4-amino-2-tetrahydropyran-2-yl-pyrazolo[4,3-c]pyridin-7-yl)-N′-[(5-fluoro-3-methyl-2-pyridyl)methyl]-N′-[(5-fluoro-2-pyridyl)methyl]oxamide (86 mg, 160.29 μmol, 27.02% yield) as a light-brown gum, which was used directly in the next step.

LCMS (ESI): [M+H]⁺m/z: calcd 537.24; found 538.2; Rt=2.890 min.

### Step 5: The synthesis of N-(4-amino-1H-pyrazolo[4,3-c]pyridin-7-yl)-N′-[(5-fluoro-3-methyl-2-pyridyl)methyl]-N′-[(5-fluoro-2-pyridyl)methyl]oxamide

Hydrogen chloride solution 4.0M in dioxane (1.58 g, 6.00 mmol, 1.5 mL, 13.9% purity) was added to a stirred solution of N-(4-amino-2-tetrahydropyran-2-yl-pyrazolo[4,3-c]pyridin-7-yl)-N′-[(5-fluoro-3-methyl-2-pyridyl)methyl]-N′-[(5-fluoro-2-pyridyl)methyl]oxamide (86 mg, 160.29 μmol) in methanol (1.5 mL) at 25° C. The resulting solution was stirred at 25° C. for 12 hr, then concentrated to dryness in vacuo and the residue was submitted to reverse phase HPLC (column: XBridge OBD C18 100×30 mm; mobile phase: 8-50% 0-1-5 min H₂O/ACN/FA; flow: 30 mL/min (loading pump 4 mL/min acetonitrile)) to afford 43 mg of crude product 91% purity by LCMS, which was repurified by reverse phase HPLC (column: XBridge C18 OBD 100×19 mm 5 μm; mobile phase: 20-60% 0-1-5 min H₂O/MeOH/NH₄OH; flow: 30 mL/min (loading pump 4 mL/min acetonitrile)) to afford N-(4-amino-1H-pyrazolo[4,3-c]pyridin-7-yl)-N′-[(5-fluoro-3-methyl-2-pyridyl)methyl]-N′-[(5-fluoro-2-pyridyl)methyl]oxamide (25 mg, 55.26 μmol, 34.47% yield) as a beige solid.

¹H NMR (600 MHz, DMSO-d₆) δ 2.16-2.33 (m, 3H), 4.61-4.74 (m, 2H), 4.99-5.11 (m, 2H), 6.50-6.71 (m, 2H), 7.40-7.58 (m, 2H), 7.59-7.65 (m, 1H), 7.65-7.74 (m, 1H), 8.10-8.16 (m, 1H), 8.28-8.36 (m, 1H), 8.42-8.51 (m, 1H), 10.30-10.46 (m, 1H), 12.70-12.86 (m, 1H).

LCMS (ESI): [M+H]⁺m/z: calcd 453.17; found 453.0; Rt=2.034 min.

### Example 108. Synthesis of N-(4-amino-1H-pyrazolo[4,3-c]pyridin-7-yl)-N′-benzyl-N′—[(2,4-dimethylphenyl)methyl]oxamide (Compound 98)

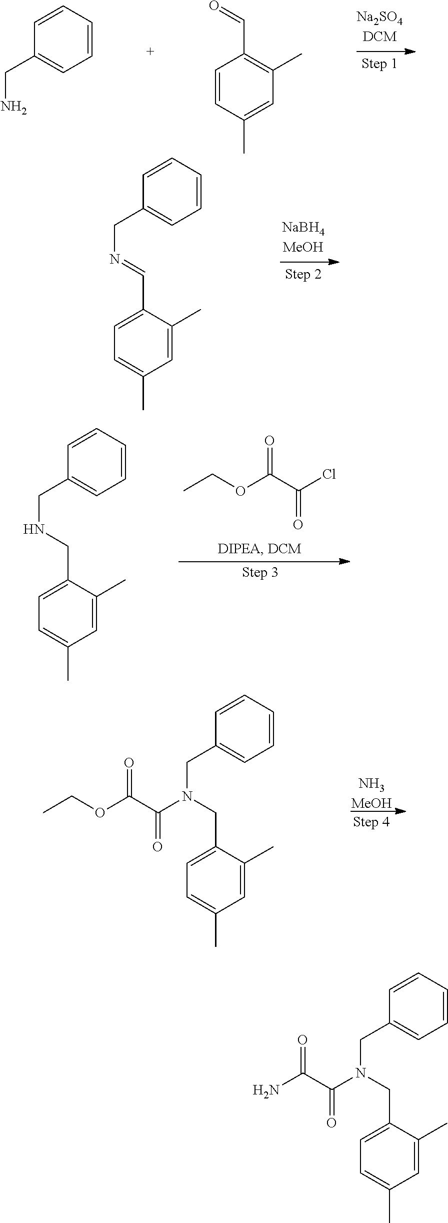

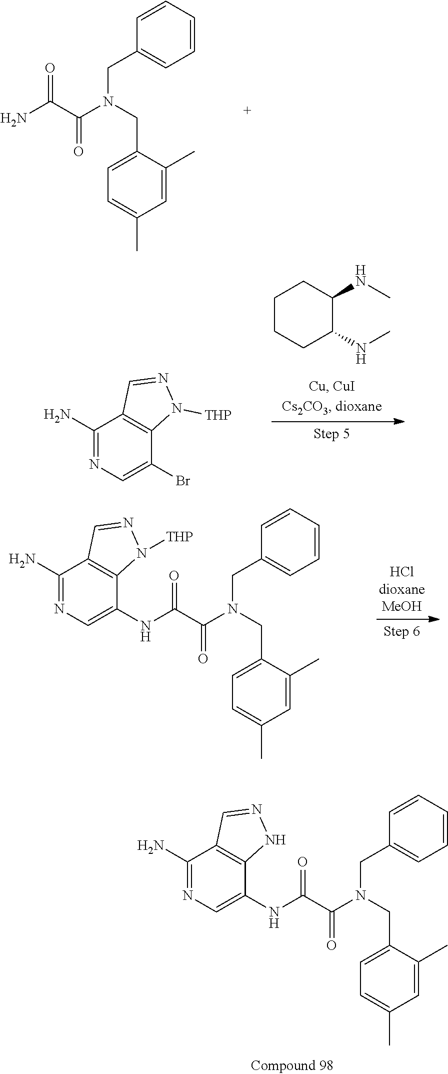

### Step 1: (E)-N-benzyl-1-(2,4-dimethylphenyl) methanimine

2,4-dimethylbenzaldehyde (5 g, 37.26 mmol), phenylmethanamine (3.99 g, 37.26 mmol) and Sodium sulfate, anhydrous (9.33 g, 65.66 mmol, 3.48 mL) was mixed in DCM (100.00 mL) and stirred at RT overnight. Upon completion, the reaction mixture was filtered and filtrate was concentrated under reduced pressure to afford (E)-N-benzyl-1-(2,4-dimethylphenyl) methanimine (8 g, crude) which was directly used in the next step without purification and analytical data collection.

### Step 2: N-[(2,4-dimethylphenyl)methyl]-1-phenyl-methanamine

(E)-N-benzyl-1-(2,4-dimethylphenyl) methanimine (8 g, 35.82 mmol) was dissolved in methanol (100 mL) followed by Sodium Borohydride (1.76 g, 46.57 mmol, 1.64 mL) addition portionwise. The reaction mixture was stirred at RT overnight and concentrated under reduced pressure. The residue was dissolved in DCM, washed with NaHCO₃ solution 3 times and concentrated to afford N-[(2,4-dimethylphenyl)methyl]-1-phenyl-methanamine (6.5 g, crude) as a yellow oil.

LCMS (ESI): [M+H]⁺m/z: calcd 226.16; found 226.2; Rt=0.879 min.

### Step 3: ethyl 2-[benzyl-[(2,4-dimethylphenyl)methyl]amino]-2-oxo-acetate

Ethyl 2-chloro-2-oxo-acetate (2.23 g, 16.31 mmol, 1.82 mL) was added dropwise to an ice bath cooled stirred solution of N-[(2,4-dimethylphenyl)methyl]-1-phenyl-methanamine (3.5 g, 15.53 mmol) and DIPEA (2.61 g, 20.19 mmol, 3.52 mL) in DCM (50 mL). The reaction mixture was stirred at RT overnight. Upon completion, the reaction mixture was washed with water. The organic layer was separated, dried over anhydrous sodium sulfate and concentrated under reduced pressure to afford ethyl 2-[benzyl-[(2,4-dimethylphenyl)methyl]amino]-2-oxo-acetate (4.5 g, crude) as a yellow oil.

LCMS (ESI): [M+H]⁺m/z: calcd 326.18; found 326.2; Rt=1.347 min.

### Step 4: N′-benzyl-N′—[(2,4-dimethylphenyl)methyl]oxamide

Ethyl 2-[benzyl-[(2,4-dimethylphenyl)methyl]amino]-2-oxo-acetate (4.5 g, 13.83 mmol) was dissolved in saturated ammonia solution and the reaction mixture was stirred at RT overnight. Upon completion, the reaction mixture was concentrated under reduced pressure to afford N′-benzyl-N′—[(2,4-dimethylphenyl)methyl]oxamide (4.34 g, crude) as a yellow oil.

### Step 5: N-(4-amino-1-tetrahydropyran-2-yl-pyrazolo[4,3-c]pyridin-7-yl)-N′-benzyl-N′—[(2,4-dimethylphenyl)methyl]oxamide

N′-benzyl-N′—[(2,4-dimethylphenyl)methyl]oxamide (0.4 g, 1.35 mmol), 7-bromo-1-tetrahydropyran-2-yl-pyrazolo[4,3-c]pyridin-4-amine (441.17 mg, 1.48 mmol), copper (45.52 mg, 716.36 μmol), Copper (I) iodide (257.05 mg, 1.35 mmol, 45.74 μL), (1S,2S)-N,N′-Bis -methyl-1,2-cyclohexane-diamine (191.98 mg, 1.35 mmol, 212.84 μL) were mixed in dioxane (20 mL). The reaction mixture was stirred under Ar atmosphere at 100° C. for 16 hr. Upon completion, the mixture was diluted with water and extracted with DCM. Organic layer was separated, dried over anhydrous sodium sulfate and concentrated under reduced pressure to afford N-(4-amino-1-tetrahydropyran-2-yl-pyrazolo[4,3-c]pyridin-7-yl)-N′-benzyl-N′—[(2,4-dimethylphenyl)methyl]oxamide (0.34 g, crude).

LCMS (ESI): [M+H]⁺m/z: calcd 513.26; found 513.4; Rt=1.010 min.

### Step 6: The synthesis of N-(4-amino-1H-pyrazolo[4,3-c]pyridin-7-yl)-N′-benzyl-N′—[(2,4-dimethylphenyl)methyl]oxamide (Compound 98)

N-(4-amino-1-tetrahydropyran-2-yl-pyrazolo[4,3-c]pyridin-7-yl)-N′-benzyl-N′—[(2,4-dimethylphenyl)methyl]oxamide (0.34 g, 663.28 μmol) was dissolved in a mixture of HCl (4.0 M in 1,4-dioxane)(24.18 mg, 663.28 μmol, 2 mL) and methanol (0.5 mL). The reaction mixture was stirred at 25° C. for 2 hr. Upon completion, obtained solution was concentrated in vacuo and the residue was submitted to HPLC (0-2-10 min 43-50-70% MeOH/H₂O+NH₄OH flow 30 mL/min (loading pump 4 mL MeOH), target mass 429, column: Chromatorex SMB100-5T 100×19, 5 microM), then repurified by HPLC (0-2-10 min 38-45-65% MeOH/H₂O+FA 30 mL/min (loading pump 4 mL MeOH), target mass 429, column: Chromatorex C18 SMB100-5T 100×19, 5microM) to afford N-(4-amino-1H-pyrazolo[4,3-c]pyridin-7-yl)-N′-benzyl-N′—[(2,4-dimethylphenyl)methyl]oxamide (0.013 g, 27.40 μmol, 4.13% yield, HCOOH) as a light-yellow solid.

LCMS (ESI): [M+H]⁺m/z: calcd 429.23; found 429.4; Rt=1.679 min.

### Example 109. Synthesis of N-(4-amino-1H-pyrazolo[4,3-c]pyridin-7-yl)-N′-[(4-fluorophenyl)methyl]-N′-(o-tolylmethyl)oxamide (Compound 173)

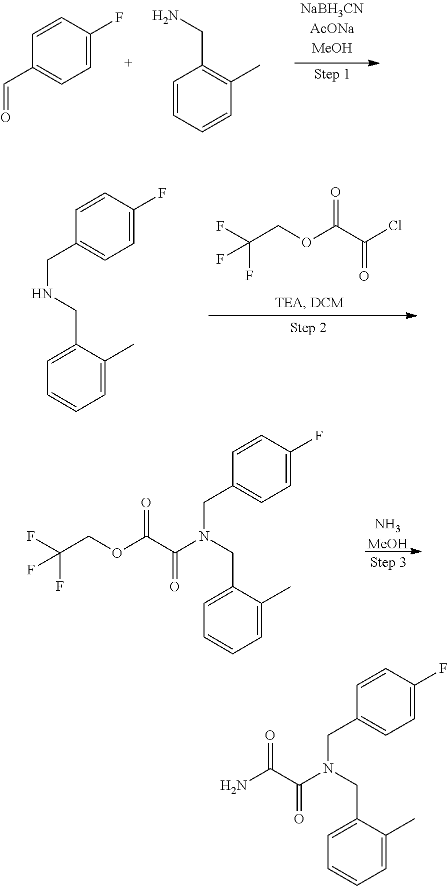

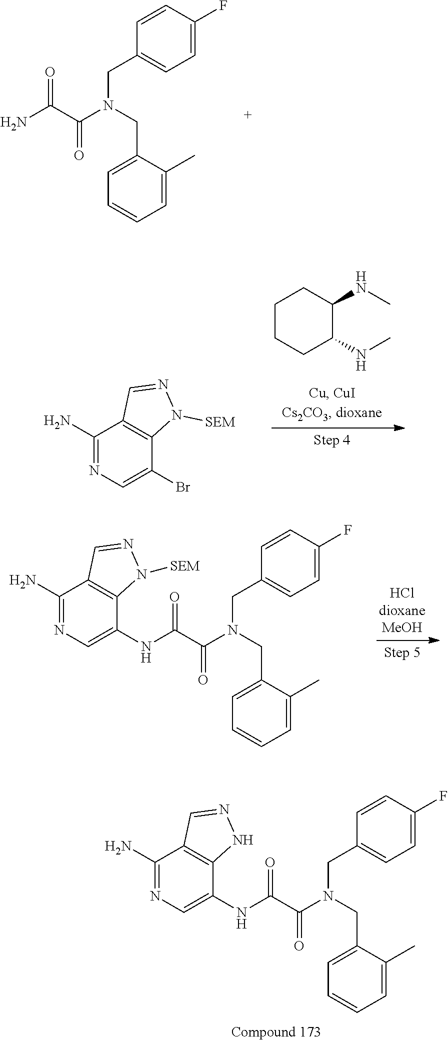

### Step 1:1-(4-fluorophenyl)-N-(o-tolylmethyl)methanamine

4-fluorobenzaldehyde (1 g, 8.06 mmol, 864.30 μL) and o-tolylmethanamine (976.37 mg, 8.06 mmol, 997.31 μL) were dissolved in MeOH (29.14 mL) and Sodium acetate (1.32 g, 16.11 mmol, 865.14 μL) was added thereto. The resulting mixture was stirred for 1 hr and Sodium cyanoborohydride (607.60 mg, 9.67 mmol) was added thereto. The resulting mixture was stirred overnight. The reaction mixture was concentrated in vacuo and aq. K₂CO₃ solution (15 mL) was added to the residue. The resulting mixture was extracted with DCM (2×10 mL) and combined organic layers were dried over anhydrous sodium sulfate, filtered and concentrated in vacuo to obtain 1-(4-fluorophenyl)-N-(o-tolylmethyl)methanamine (1.5 g, 6.54 mmol, 81.19% yield) as a light-yellow oil.

LCMS (ESI): [M+H]⁺m/z: calcd 230.14; found 230.2; Rt=0.806 min.

### Step 2:2,2,2-trifluoroethyl 2-[(4-fluorophenyl)methyl-(o-tolylmethyl)amino]-2-oxo-acetate

1-(4-fluorophenyl)-N-(o-tolylmethyl)methanamine (1.50 g, 6.54 mmol) was dissolved in DCM (27.72 mL) and TEA (1.65 g, 16.35 mmol, 2.28 mL) was added. The reaction mixture was cooled and 2,2,2-trifluoroethyl 2-chloro-2-oxo-acetate (1.50 g, 7.85 mmol) was added dropwise. After that it was stirred at rt overnight. Then water (10 mL) was added, organic layer was stirred with brine and dried over anhydrous sodium sulfate, evaporated in vacuum to afford 2,2,2-trifluoroethyl 2-[(4-fluorophenyl)methyl-(o-tolylmethyl)amino]-2-oxo-acetate (1.8 g, 4.70 mmol, 71.78% yield) as a light-yellow oil.

LCMS (ESI): [M+H]⁺m/z: calcd 384.12; found 384.2; Rt=1.526 min.

### Step 3: N′-[(4-fluorophenyl)methyl]-N′-(o-tolylmethyl)oxamide

2,2,2-trifluoroethyl 2-[(4-fluorophenyl)methyl-(o-tolylmethyl)amino]-2-oxo-acetate (1.8 g, 4.70 mmol) was dissolved in NH3/MeOH (20 mL) and stirred overnight at rt. Then it was evaporated in vacuum and subjected to CC (CHCl₃-MeCN was used as an eluent mixture) to afford N′-[(4-fluorophenyl)methyl]-N′-(o-tolylmethyl)oxamide (0.7 g, 2.33 mmol, 49.64% yield) as a beige solid.

LCMS (ESI): [M+Na]⁺m/z: calcd 323.13; found 323.0; Rt=1.298 min.

### Step 4: N-[4-amino-1-(2-trimethylsilylethoxymethyl)pyrazolo[4,3-c]pyridin-7-yl]-N′-[(4-fluorophenyl)methyl]-N′-(o-tolylmethyl)oxamide

A mixture of 7-bromo-1-(2-trimethylsilylethoxymethyl)pyrazolo[4,3-c]pyridin-4-amine (571.53 mg, 1.66 mmol), N′-[(4-fluorophenyl)methyl]-N′-(o-tolylmethyl)oxamide (0.5 g, 1.66 mmol)N′-[(4-fluorophenyl)methyl]-N′-(o-tolylmethyl)oxamide (0.5 g, 1.66 mmol) Cesium carbonate (813.66 mg, 2.50 mmol), rac-(1R,2R)—N1,N2-dimethylcyclohexane-1,2-diamine (189.45 mg, 1.33 mmol) and CuI (190.24 mg, 998.91 μmol, 33.85 μL) with a few mg of Cu(5.29 mg, 83.24 μmol) in dioxane (4.97 mL) was stirred in a sealed vial under argon at 105° C. for 48 hr. The resulting mixture was cooled down and filtered through silica gel to afford N-[4-amino-1-(2-trimethylsilylethoxymethyl)pyrazolo[4,3-c]pyridin-7-yl]-N′-[(4-fluorophenyl)methyl]-N′-(o-tolylmethyl)oxamide (0.9 g, 1.60 mmol, 96.07% yield) as a brown gum.

LCMS (ESI): [M+H]⁺m/z: calcd 563.26; found 563.4; Rt=1.447 min.

### Step 5: The synthesis of N-(4-amino-1H-pyrazolo[4,3-c]pyridin-7-yl)-N′-[(4-fluorophenyl)methyl]-N′-(o-tolylmethyl)oxamide

N-[4-amino-1-(2-trimethylsilylethoxymethyl)pyrazolo[4,3-c]pyridin-7-yl]-N′-[(4-fluorophenyl)methyl]-N′-(o-tolylmethyl)oxamide (0.9 g, 1.60 mmol) was dissolved in MeOH (10 mL) and diox/HCl (1.60 mmol, 5 mL) was added. Then mixture was stirred at rt 12 hr. The solution was evaporated and submitted to reverse phase HPLC (column: SunFire C18 100×19 mm, 5 μm; mobile phase: 30-40-65% 0-2-8 min water-MeOH+FA, flow rate: 30 mL/min) to afford N-(4-amino-1H-pyrazolo[4,3-c]pyridin-7-yl)-N′-[(4-fluorophenyl)methyl]-N′-(o-tolylmethyl)oxamide (153.5 mg, 354.95 μmol, 22.19% yield) as a beige solid.

¹H NMR (600 MHz, DMSO-d₆) δ 1.99-2.19 (m, 3H), 4.38-4.53 (m, 2H), 4.57-4.80 (m, 2H), 7.11-7.23 (m, 6H), 7.25-7.39 (m, 2H), 7.78-7.90 (m, 1H), 7.90-8.73 (m, 3H), 10.77-11.03 (m, 1H), 12.60-13.78 (m, 1H).

LCMS (ESI): [M+H]⁺m/z: calcd 434.0; found 434.2; Rt=2.665 min.

### Example 110. Synthesis of N-(4-amino-1H-pyrazolo[4,3-c]pyridin-7-yl)-N′-[(2-isopropylphenyl)methyl]-N′-methyl-oxamide (Compound 183)

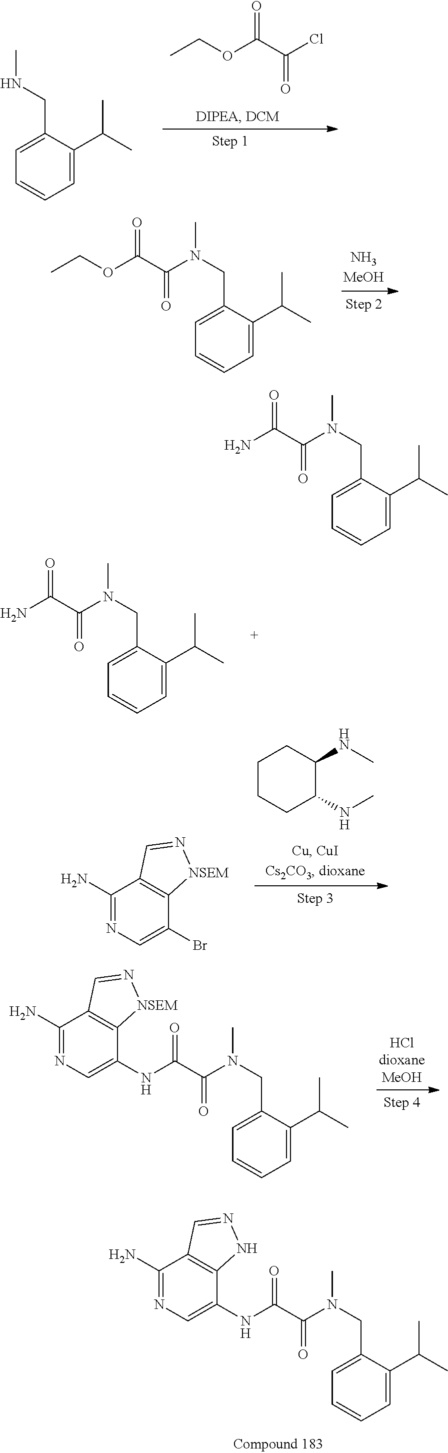

### Step 1: ethyl 2-[(2-isopropylphenyl)methyl-methyl-amino]-2-oxo-acetate

Ethyl 2-chloro-2-oxo-acetate (1.44 g, 10.51 mmol, 1.17 mL) was added dropwise to an ice bath cooled stirred solution of 1-(2-isopropylphenyl)-N-methyl-methanamine (2 g, 10.01 mmol, HCl) and DIPEA (3.24 g, 25.04 mmol, 4.36 mL) in DCM (60 mL). The reaction mixture was stirred at RT overnight. Upon completion, the reaction mixture was washed with water. The organic layer was separated, dried over anhydrous sodium sulfate and concentrated under reduced pressure to afford ethyl 2-[(2-isopropylphenyl)methyl-methyl-amino]-2-oxo-acetate (2.3 g, crude).

LCMS (ESI): [M+H]⁺m/z: calcd 264.16; found 264.2; Rt=1.065 min.

### Step 2: N′-[(2-isopropylphenyl)methyl]-N′-methyl-oxamide

Ethyl 2-[(2-isopropylphenyl)methyl-methyl-amino]-2-oxo-acetate (2.3 g, 8.73 mmol) was dissolved in saturated methanol/ammonia solution and the reaction mixture was stirred overnight at RT. Upon completion, the mixture was concentrated under reduced pressure to afford N′-[(2-isopropylphenyl)methyl]-N′-methyl-oxamide (2 g, crude) as a light-yellow solid.

LCMS (ESI): [M−H]⁻ m/z: calcd 233.13; found 233.0; Rt=0.983 min.

### Step 3: N-[4-amino-1-(2-trimethylsilylethoxymethyl)pyrazolo[4,3-c]pyridin-7-yl]-N′-[(2-isopropylphenyl)methyl]-N′-methyl-oxamide

N′-[(2-isopropylphenyl)methyl]-N′-methyl-oxamide (0.5 g, 2.13 mmol), 7-bromo-1-(2-trimethylsilylethoxymethyl)pyrazolo[4,3-c]pyridin-4-amine (732.62 mg, 2.13 mmol), copper (71.98 mg, 1.13 mmol), Copper (I) iodide (406.43 mg, 2.13 mmol, 72.32 μL), (1S,2S)-N,N′-Bis -methyl-1,2-cyclohexane-diamine (303.55 mg, 2.13 mmol, 336.53 μL) were mixed in dioxane (20 mL). The reaction mixture was stirred under Ar atmosphere at 100° C. for 16 hr. Upon completion, the mixture was diluted with water and extracted with DCM. Organic layer was separated, dried over anhydrous sodium sulfate and concentrated under reduced pressure. The residue was submitted to HPLC (0-2-10 min 33-40-65% H₂O/ACN/0.1% FA flow 30 mL/min ((loading pump 4 mL ACN) column: Chromatorex SMB100-5T C18 100×19 mm, 5 microM) to afford N-[4-amino-1-(2-trimethylsilylethoxymethyl)pyrazolo[4,3-c]pyridin-7-yl]-N′-[(2-isopropylphenyl)methyl]-N′-methyl-oxamide (0.13 g, 261.74 μmol, 12.26% yield).

LCMS (ESI): [M+H]⁺m/z: calcd 497.32; found 497.2; Rt=1.516 min.

### Step 4: The synthesis of N-(4-amino-1H-pyrazolo[4,3-c]pyridin-7-yl)-N′-[(2-isopropylphenyl)methyl]-N′-methyl-oxamide

N-[4-amino-1-(2-trimethylsilylethoxymethyl)pyrazolo[4,3-c]pyridin-7-yl]-N′-[(2-isopropylphenyl)methyl]-N′-methyl-oxamide (0.13 g, 261.74 μmol) was dissolved in a mixture of HCl/dioxane solution (2 mL) and methanol (0.5 mL) and stirred at 25° C. for 4 hr. Upon completion, the reaction mixture was concentrated and the residue was purified by reverse phase HPLC (0-2-10 min 0-5-50% MeOH/H₂O+FA, flow 30 mL/min (loading pump 4 mL/min MeOH), target MI 367 Column: Chromatorex C18 5 μm 100×19 mm) to afford N-(4-amino-1H-pyrazolo[4,3-c]pyridin-7-yl)-N′-[(2-isopropylphenyl)methyl]-N′-methyl-oxamide (0.06 g, 145.48 μmol, 55.58% yield, HCOOH) as a yellow solid.

¹H NMR (600 MHz, DMSO-d₆) § 1.06-1.19 (m, 6H), 2.79-3.04 (m, 3H), 3.14-3.18 (m, 1H), 4.65-4.94 (m, 2H), 6.64-6.73 (m, 2H), 7.16-7.22 (m, 2H), 7.23-7.37 (m, 2H), 7.60-7.73 (m, 1H), 8.15-8.21 (m, 1H), 9.68-10.50 (m, 1H), 12.80 (s, 1H).

LCMS (ESI): [M+H]⁺m/z: calcd 367.21; found 367.4; Rt=1.352 min.

### Example 111. Synthesis of N-(4-amino-2H-pyrazolo[4,3-c]pyridin-7-yl)-N′-(1-naphthylmethyl)-N′-(2-pyridylmethyl)oxamide (Compound 132)

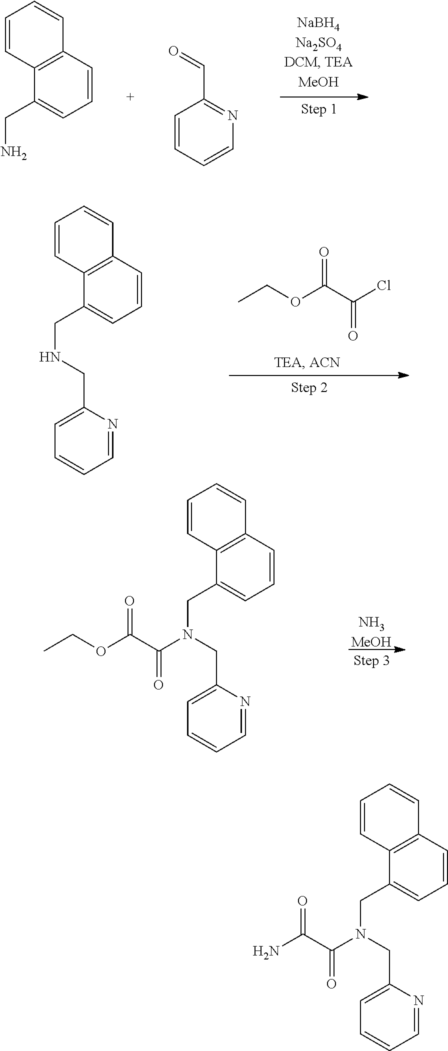

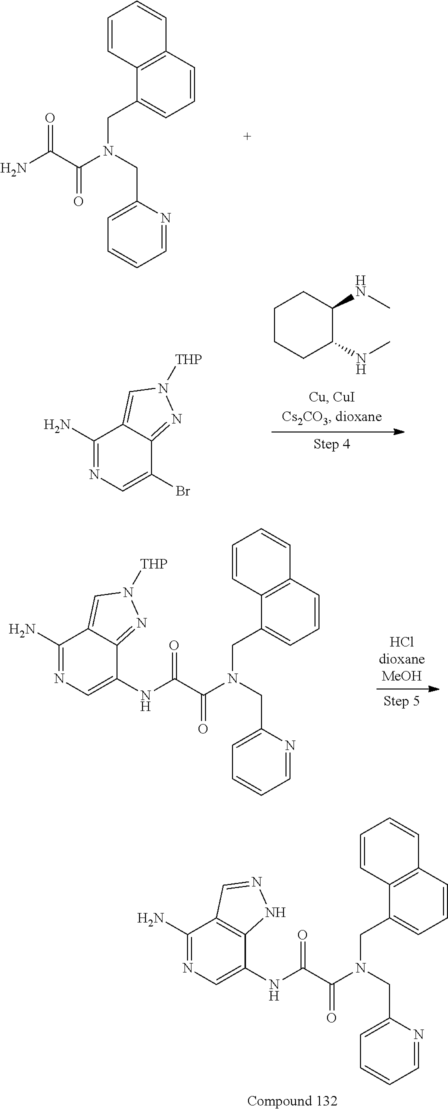

### Step 1:1-(1-naphthyl)-N-(2-pyridylmethyl)methanamine

1-naphthylmethanamine (1 g, 5.16 mmol, 930.23 μL, HCl), TEA (574.73 mg, 5.68 mmol, 791.64 μL), Sodium sulfate, anhydrous (733.40 mg, 5.16 mmol, 273.66 μL) and pyridine-2-carbaldehyde (553.05 mg, 5.16 mmol, 492.03 μL) were mixed in DCM (20 mL) at 20° C. The resulting mixture was stirred at 20° C. for 12 hr, then filtered and concentrated in vacuo. The residue was dissolved in methanol (20 mL), cooled to 5° C. and Sodium Borohydride (214.86 mg, 5.68 mmol, 200.06 μL) was added. The reaction mixture was allowed to warm to 20° C. and stirred for 2 hr, then concentrated in vacuo. The residue was diluted with water (25 mL) and extracted with dichloromethane (40 mL). The organic layer was separated, dried over anhydrous sodium sulfate and concentrated in vacuo to afford 1-(1-naphthyl)-N-(2-pyridylmethyl)methanamine (1.06 g, 4.27 mmol, 82.67% yield) as a brown liquid.

LCMS (ESI): [M+H]⁺m/z: calcd 249.14; found 249.2; Rt=0.794 min.

### Step 2: ethyl 2-[1-naphthylmethyl(2-pyridylmethyl)amino]-2-oxo-acetate

1-(1-naphthyl)-N-(2-pyridylmethyl)methanamine (1.06 g, 4.27 mmol) and TEA (863.89 mg, 8.54 mmol, 1.19 mL) were dissolved in acetonitrile (20.91 mL), cooled with ice-water bath and then ethyl 2-chloro-2-oxoacetate (757.66 mg, 5.55 mmol, 620.02 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-[1-naphthylmethyl(2-pyridylmethyl)amino]-2-oxo-acetate (1.3 g, crude) as a brown liquid.

LCMS (ESI): [M+H]⁺m/z: calcd 349.16; found 349.2; Rt=1.400 min.

### Step 3: N′-(1-naphthylmethyl)-N′-(2-pyridylmethyl)oxamide

Ethyl 2-[1-naphthylmethyl(2-pyridylmethyl)amino]-2-oxo-acetate (1.3 g, 3.73 mmol) was dissolved in MeOH (20 mL) saturated with NH3 (3.73 mmol) and stirred overnight. The RM was concentrated, redissolved in DCM, washed with water, dried over anhydrous sodium sulfate and concentrated in vacuo to give N′-(1-naphthylmethyl)-N′-(2-pyridylmethyl)oxamide (0.73 g, crude) as a brown solid.

LCMS (ESI): [M+H]⁺m/z: calcd 320.14; found 320.1; Rt=1.074 min.

### Step 4: N-(4-amino-2-tetrahydropyran-2-yl-pyrazolo[4,3-c]pyridin-7-yl)-N′-(1-naphthylmethyl)-N′-(2-pyridylmethyl)oxamide

Copper (3.98 mg, 62.63 μmol), Copper (I) iodide (35.78 mg, 187.88 μmol, 6.37 μL), cesium carbonate (408.10 mg, 1.25 mmol) were added to a stirred solution of N′-(1-naphthylmethyl)-N′-(2-pyridylmethyl)oxamide (200 mg, 626.26 μmol), 7-bromo-2-tetrahydropyran-2-yl-pyrazolo[4,3-c]pyridin-4-amine (223.31 mg, 751.51 μmol) and rac-(1R,2R)—N1,N2-dimethylcyclohexane-1,2-diamine (44.54 mg, 313.13 μmol) in 1,4-dioxane (5.00 mL) under Ar atmosphere and stirred at 100° C. for 12 hr in closed vial. The reaction mixture was filtered and evaporated in vacuo to give N-(4-amino-2-tetrahydropyran-2-yl-pyrazolo[4,3-c]pyridin-7-yl)-N′-(1-naphthylmethyl)-N′-(2-pyridylmethyl)oxamide (0.45 g, crude) as a brown oil.

LCMS (ESI): [M+H]⁺m/z: calcd 536.24; found 536.2; Rt=1.030 min.

### Step 5: The synthesis of N-(4-amino-2H-pyrazolo[4,3-c]pyridin-7-yl)-N′-(1-naphthylmethyl)-N′-(2-pyridylmethyl)oxamide

N-(4-amino-2-tetrahydropyran-2-yl-pyrazolo[4,3-c]pyridin-7-yl)-N′-(1-naphthylmethyl)-N′-(2-pyridylmethyl)oxamide (0.45 g, 197.44 μmol) was dissolved in Dioxane (4 mL) saturated with HCl (10% by weight). MeOH (4 mL) was added for better solubility. The RM was stirred for 12 hr at 20° C., then concentrated in vacuo. The residue was dissolved in 3 mL of MeOH and subjected to HPLC (Device (Mobile Phase, Column): SYSTEM 0-2-8 min May 15, 2045% H₂O+HCl/ACN flow 30 mL/min (loading pump 4 mL/min ACN) to afford N-(4-amino-2H-pyrazolo[4,3-c]pyridin-7-yl)-N′-(1-naphthylmethyl)-N′-(2-pyridylmethyl)oxamide (24.6 mg, 50.42 μmol, 25.53% yield, HCl) as a light-brown solid.

¹H NMR (600 MHz, DMSO-d₆) δ 4.58-4.89 (m, 2H), 4.89-5.50 (m, 2H), 6.55-7.08 (m, 2H), 7.25-7.30 (m, 1H), 7.33-7.40 (m, 2H), 7.45-7.52 (m, 2H), 7.54-7.56 (m, 1H), 7.59-7.67 (m, 1H), 7.71-7.78 (m, 1H), 7.83-7.89 (m, 1H), 7.92-7.99 (m, 1H), 8.03-8.09 (m, 1H), 8.12-8.18 (m, 1H), 8.48-8.53 (m, 1H), 9.70-10.78 (m, 1H), 12.63-13.01 (m, 1H).

LCMS (ESI): [M+H]⁺m/z: calcd 452.2; found 453.2; Rt=2.282 min.

### Example 112. Synthesis of N-(4-amino-1H-pyrazolo[4,3-c]pyridin-7-yl)-N′-ethyl-N′-(1-naphthylmethyl)oxamide (Compound 176)

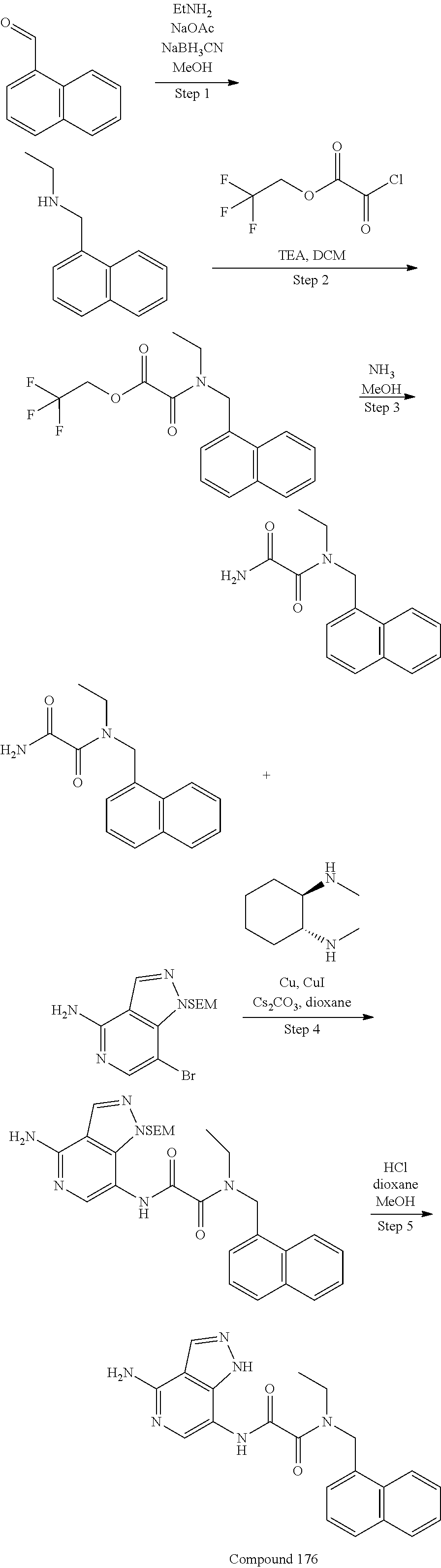

### Step 1: N-(1-naphthylmethyl)ethanamine

Naphthalene-1-carbaldehyde (1 g, 6.40 mmol, 869.57 μL) and ethanamine (522.12 mg, 6.40 mmol, 650.21 μL, HCl) were dissolved in MeOH (29.14 mL) and Sodium acetate (1.05 g, 12.81 mmol, 687.50 μL) was added thereto. The resulting mixture was stirred for 1 hr and Sodium cyanoborohydride (482.84 mg, 7.68 mmol) was added thereto. The resulting mixture was stirred overnight. The reaction mixture was concentrated in vacuo and aq. K₂CO₃ solution (15 mL) was added to the residue. The resulting mixture was extracted with DCM (2×10 mL) and combined organic layers were dried over anhydrous sodium sulfate, filtered and concentrated in vacuo to obtain N-(1-naphthylmethyl)ethanamine (1.1 g, 5.94 mmol, 92.73% yield) as a yellow oil.

LCMS (ESI): [M+H]⁺m/z: calcd 186.13; found 186.2; Rt=0.863 min.

### Step 2:2,2,2-trifluoroethyl 2-[ethyl(1-naphthylmethyl)amino]-2-oxo-acetate

N-(1-naphthylmethyl)ethanamine (1.1 g, 5.94 mmol) was dissolved in DCM (20 mL) and TEA (1.50 g, 14.84 mmol, 2.07 mL) was added. The reaction mixture was cooled and 2,2,2-trifluoroethyl 2-chloro-2-oxo-acetate (1.13 g, 5.94 mmol) was added dropwise. After that it was stirred at rt overnight. Then water (10 mL) was added, organic layer was stirred with brine and dried over anhydrous sodium sulfate, evaporated in vacuum to afford 2,2,2-trifluoroethyl 2-[ethyl(1-naphthylmethyl)amino]-2-oxo-acetate (1.8 g, 5.30 mmol, 89.35% yield) as a yellow oil.

LCMS (ESI): [M+H]⁺m/z: calcd 240.12; found 240.2; Rt=1.287 min.

### Step 3: N′-ethyl-N′-(1-naphthylmethyl)oxamide

2,2,2-trifluoroethyl 2-[ethyl(1-naphthylmethyl)amino]-2-oxo-acetate (1.8 g, 5.30 mmol) was dissolved in NH₃/MeOH (20 mL) and stirred overnight at rt. Then it was evaporated in vacuum and subjected to CC (CHCl₃-MeCN was used as an eluent mixture) to afford N′-ethyl-N′-(1-naphthylmethyl)oxamide (0.5 g, 1.95 mmol, 36.77% yield) as a yellow oil.

LCMS (ESI): [M+Na]⁺m/z: calcd 279.12; found 279.0; Rt=1.053 min.

### Step 4: N-[4-amino-1-(2-trimethylsilylethoxymethyl)pyrazolo[4,3-c]pyridin-7-yl]-N′-ethyl-N′-(1-naphthylmethyl)oxamide

A mixture of 7-bromo-1-(2-trimethylsilylethoxymethyl)pyrazolo[4,3-c]pyridin-4-amine (294.67 mg, 858.37 μmol), N′-ethyl-N′-(1-naphthylmethyl)oxamide (0.2 g, 780.34 μmol), Cesium carbonate (381.37 mg, 1.17 mmol), rac-(1R,2R)—N1,N2-dimethylcyclohexane-1,2-diamine (88.80 mg, 624.27 μmol) and CuI (89.17 mg, 468.20 μmol, 15.87 μL) with a few mg of Cu(2.48 mg, 39.02 μmol) in dioxane (3.98 mL) was stirred in a sealed vial under argon at 110° C. for 48 hr. The resulting mixture was cooled down and filtered through silica gel to afford N-[4-amino-1-(2-trimethylsilylethoxymethyl)pyrazolo[4,3-c]pyridin-7-yl]-N′-ethyl-N′-(1-naphthylmethyl)oxamide (0.4 g, 771.19 μmol, 98.83% yield) as a brown gum.

LCMS (ESI): [M+H]⁺m/z: calcd 519.26; found 519.2; Rt=1.576 min.

### Step 5: The synthesis of N-(4-amino-1H-pyrazolo[4,3-c]pyridin-7-yl)-N′-ethyl-N′-(1-naphthylmethyl)oxamide

N-[4-amino-1-(2-trimethylsilylethoxymethyl)pyrazolo[4,3-c]pyridin-7-yl]-N′-ethyl-N′-(1-naphthylmethyl)oxamide (0.4 g, 771.19 μmol) was dissolved in MeOH (10 mL) and dioxane/HCl (771.19 μmol, 1 mL) was added. Then mixture was stirred at rt 12 hr. The solution was evaporated and submitted to reverse phase HPLC (column: SunFire C18 100×19 mm, 5 μm; mobile phase: 20-45% 2-7.5 min water-MeCN+HCl, flow rate: 30 mL/min) to afford N-(4-amino-1H-pyrazolo[4,3-c]pyridin-7-yl)-N′-ethyl-N′-(1-naphthylmethyl)oxamide (12.9 mg, 30.36 μmol, 3.94% yield, HCl) as a light-yellow solid.

¹H NMR (600 MHz, DMSO-d₆) δ 0.77-1.19 (m, 3H), 3.37-3.59 (m, 2H), 5.04-5.76 (m, 2H), 7.24-7.54 (m, 2H), 7.54-7.60 (m, 1H), 7.74-7.90 (m, 1H), 7.90-8.01 (m, 1H), 8.01-8.17 (m, 1H), 8.23-10.07 (m, 4H), 10.83-11.20 (m, 1H), 12.95 (br s, 1H), 13.87-15.00 (m, 1H).

LCMS (ESI): [M+H]⁺m/z: calcd 389.19; found 389.0; Rt=2.372 min.

### Example 113. Synthesis of N-(4-amino-1H-pyrazolo[4,3-c]pyridin-7-yl)-N′-[(4-chlorophenyl)methyl]-N′-(o-tolylmethyl)oxamide (Compound 109)

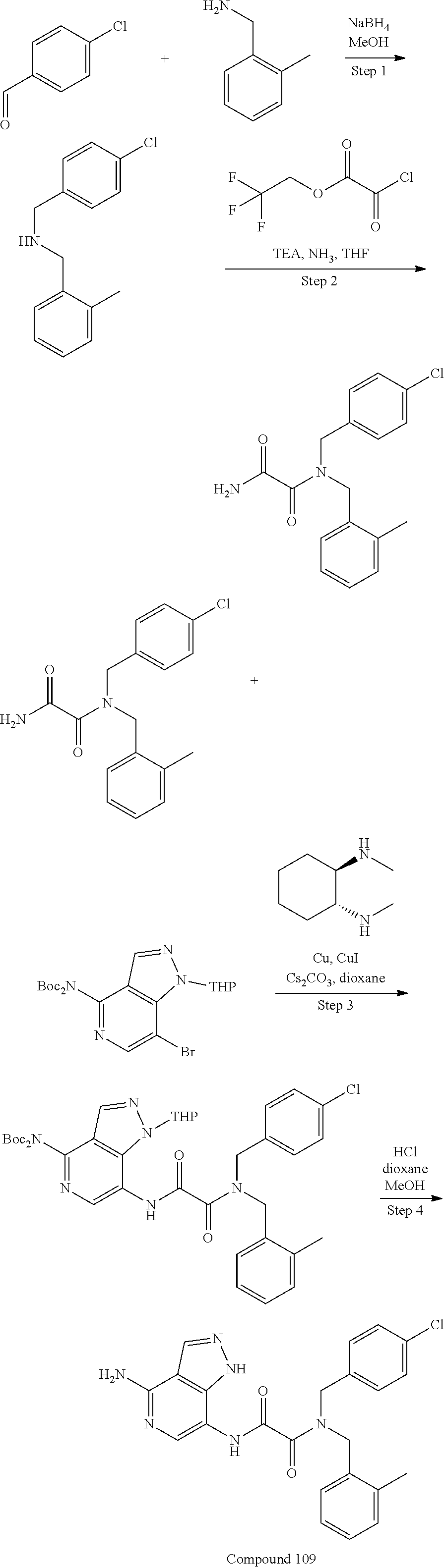

### Step 1:1-(4-chlorophenyl)-N-(o-tolylmethyl)methanamine

A solution of 4-chlorobenzaldehyde (2.32 g, 16.50 mmol) and o-tolylmethanamine (2 g, 16.50 mmol, 2.04 mL) in methanol (30 mL) was stirred at 25° C. for 12 hr. To this solution, Sodium Borohydride (624.36 mg, 16.50 mmol, 581.35 μL) was added and the resulting mixture was stirred for 12 hr. The solvent was removed in vacuo, the residue was taken up with water (20 mL) and extracted with DCM (3×30 mL). The combined organic layer was washed with brine (20 mL), dried over anhydrous sodium sulfate and evaporated to obtain 1-(4-chlorophenyl)-N-(o-tolylmethyl)methanamine (4 g, 16.28 mmol, 98.62% yield) as a yellow oil.

LCMS (ESI): [M+H]⁺m/z: calcd 246.11; found 246.2; Rt=1.034 min.

### Step 2: N′-[(4-chlorophenyl)methyl]-N′-(o-tolylmethyl)oxamide

To a solution of 1-(4-chlorophenyl)-N-(o-tolylmethyl)methanamine (2.95 g, 12.00 mmol) and TEA (2.43 g, 24.01 mmol, 3.35 mL) in THF (25 mL) was added 2,2,2-trifluoroethyl 2-chloro-2-oxo-acetate (3.43 g, 18.01 mmol) dropwise at 0° C. under argon. The reaction mixture was then stirred for 12 hr at r.t. Then ammonia (292.20 mg, 17.16 mmol) was bubbled through for 10 min at 0° C. The reaction mixture was then stirred for 12 hr at r.t. The reaction mixture was filtered off and the filtrate was evaporated in vacuo to give N′-[(4-chlorophenyl)methyl]-N′-(o-tolylmethyl)oxamide (2 g, 6.31 mmol, 52.59% yield) as a brown solid.

LCMS (ESI): [M−H]⁻ m/z: calcd 315.09; found 315.0; Rt=1.074 min.

### Step 3: tert-butyl N-tert-butoxycarbonyl-N-[7-[[2-[(4-chlorophenyl)methyl-(0-tolylmethyl)amino]-2-oxo-acetyl]amino]-1-tetrahydropyran-2-yl-pyrazolo[4,3-c]pyridin-4-yl]carbamate

N′-[(4-chlorophenyl)methyl]-N′-(o-tolylmethyl)oxamide (0.3 g, 947.03 μmol), tert-butyl N-(7-bromo-1-tetrahydropyran-2-yl-pyrazolo[4,3-c]pyridin-4-yl)-N-tert-butoxycarbonyl-carbamate (518.14 mg, 1.04 mmol), Cu(6.38 mg, 100.38 μmol), CuI (54.11 mg, 284.11 μmol, 9.63 μL), Cesium carbonate (462.84 mg, 1.42 mmol, 202.11 μL) and (1R,2R)—N1,N2-dimethylcyclohexane-1,2-diamine (40.41 mg, 284.11 μmol) were mixed in dioxane (4 mL), purged with Ar for 5 minutes and then heated in the closed reaction vial at 100° C. for 24 hr. Final mixture was filtered and dioxane was evaporated in vacuo to afford tert-butyl N-tert-butoxycarbonyl-N-[7-[2-[(4-chlorophenyl)methyl-(o-tolylmethyl)amino]-2-oxo-acetyl]amino]-1-tetrahydropyran-2-yl-pyrazolo[4,3-c]pyridin-4-yl]carbamate (1 g, crude) as a brown solid which was used directly in the next step.

LCMS (ESI): [M+H]⁺m/z: calcd 733.31; found 733.2; Rt=1.671 min.

### Step 4: The synthesis of N-(4-amino-1H-pyrazolo[4,3-c]pyridin-7-yl)-N′-[(4-chlorophenyl)methyl]-N′-(o-tolylmethyl)oxamide

To a solution of tert-butyl N-tert-butoxycarbonyl-N-[7-[2-[(4-chlorophenyl)methyl-(o-tolylmethyl)amino]-2-oxo-acetyl]amino]-1-tetrahydropyran-2-yl-pyrazolo[4,3-c]pyridin-4-yl]carbamate (648.00 mg, 883.73 μmol) in methanol (10 mL) was added Hydrogen chloride solution 4.0M in dioxane (4.83 g, 13.26 mmol, 4.60 mL, 10% purity) at 21° C. The resulting mixture was left to stir for 32 hr. The resulting mixture was evaporated to dryness. The residue was purified by RP-HPLC (column: Chromatorex 18 SMB 100-ST 100×19 mm 5 μm; 15-15-45% 0-1.3-5.3 min H₂O/ACN/0.1% FA, flow: 30 mL/min) to give N-(4-amino-1H-pyrazolo[4,3-c]pyridin-7-yl)-N′-[(4-chlorophenyl)methyl]-N′-(o-tolylmethyl)oxamide (53 mg, 107.09 μmol, 12.12% yield, HCOOH) as a yellow solid.

¹H NMR (500 MHz, DMSO-d₆) δ 1.80-2.21 (m, 3H), 4.00-4.88 (m, 4H), 6.72-7.24 (m, 6H), 7.26-8.15 (m, 5H), 8.18-8.35 (m, 1H), 9.59-10.86 (m, 1H), 12.82-13.55 (m, 1H).

LCMS (ESI): [M+H]⁺m/z: calcd 449.17; found 449.2; Rt=3.038 min.

### Example 114. Synthesis of N-(4-amino-2H-pyrazolo[4,3-c]pyridin-7-yl)-N′—[(2,3-dichlorophenyl)methyl]-N′-(2-pyridylmethyl)oxamide (Compound 91)

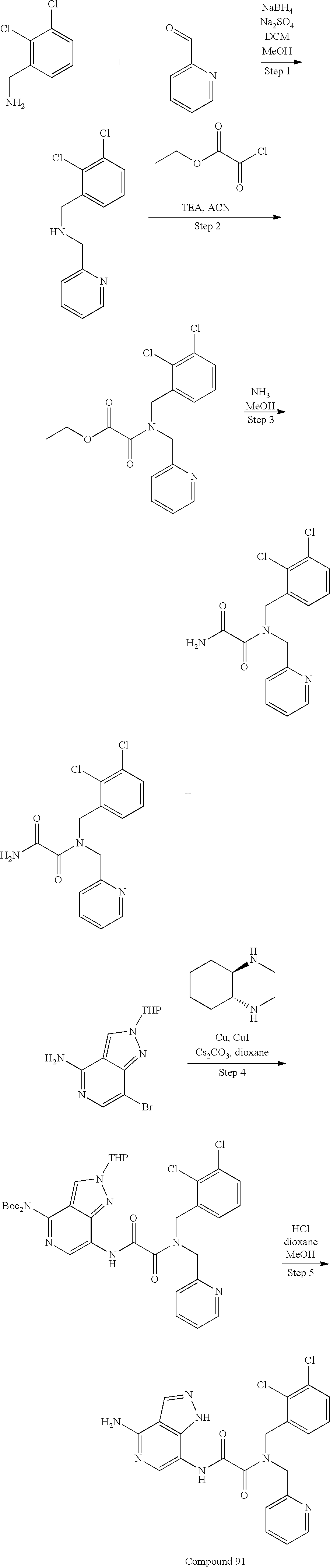

### Step 1:1-(2,3-dichlorophenyl)-N-(2-pyridylmethyl)methanamine

(2,3-dichlorophenyl)methanamine (1 g, 5.68 mmol, 757.00 μL), Sodium sulfate, anhydrous (806.85 mg, 5.68 mmol, 301.06 μL) and pyridine-2-carbaldehyde (608.43 mg, 5.68 mmol, 541.31 μL) were mixed in DCM (19.91 mL) at 20° C. The resulting mixture was stirred at 2° C. for 12 hr, then filtered and concentrated in vacuo. The residue was dissolved in methanol (19.91 mL), cooled to 5° C. and Sodium Borohydride (236.38 mg, 6.25 mmol, 220.09 μL) was added. The reaction mixture was allowed to warm to 20° C. and stirred for 2 hr, then concentrated in vacuo. The residue was diluted with water (25 mL) and extracted with DCM (40 mL). The organic layer was separated, dried over anhydrous sodium sulfate and concentrated in vacuo to afford 1-(2,3-dichlorophenyl)-N-(2-pyridylmethyl)methanamine (1.47 g, 5.50 mmol, 96.87% yield) as a brown liquid.

LCMS (ESI): [M+H]⁺m/z: calcd 267.05; found 267.0; Rt=0.781 min.

### Step 2: ethyl 2-[(2,3-dichlorophenyl)methyl-(2-pyridylmethyl)amino]-2-oxo-acetate

1-(2,3-dichlorophenyl)-N-(2-pyridylmethyl)methanamine (1.47 g, 5.50 mmol) and TEA (1.11 g, 11.00 mmol, 1.53 mL) were dissolved in acetonitrile (20 mL), cooled with ice-water bath and then ethyl 2-chloro-2-oxoacetate (976.65 mg, 7.15 mmol, 799.22 μL) was added. Solution was stirred overnight at 20° C. The RM was concentrated in vacuo, redissolved in DCM (40 mL), and washed with water twice. Organic phase was dried over anhydrous sodium sulfate and concentrated in vacuo to give ethyl 2-[(2,3-dichlorophenyl)methyl-(2-pyridylmethyl)amino]-2-oxo-acetate (1.8 g, crude) as a brown liquid.

LCMS (ESI): [M+H]⁺m/z: calcd 367.06; found 367.0; Rt=1.258 min.

### Step 3: N′—[(2,3-dichlorophenyl)methyl]-N′-(2-pyridylmethyl)oxamide

Ethyl 2-[(2,3-dichlorophenyl)methyl-(2-pyridylmethyl)amino]-2-oxo-acetate (1.8 g, 3.92 mmol) was dissolved in MeOH (20 mL) saturated with NH3 (3.92 mmol) and stirred overnight. The RM was concentrated, dissolved in DCM, washed with water, dried over anhydrous sodium sulfate and concentrated in vacuo to give N′—[(2,3-dichlorophenyl)methyl]-N′-(2-pyridylmethyl)oxamide (1.2 g, 3.55 mmol, 90.49% yield) as a brown solid.

LCMS (ESI): [M+H]⁺m/z: calcd 338.05; found 338.0; Rt=1.139 min.

### Step 4: tert-butyl N-tert-butoxycarbonyl-N-[7-[[2-[(2,3-dichlorophenyl)methyl-(2-pyridylmethyl)amino]-2-oxo-acetyl]amino]-2-tetrahydropyran-2-yl-pyrazolo[4,3-c]pyridin-4-yl]carbamate

Copper (16.91 mg, 266.12 μmol), Copper (I) iodide (168.94 mg, 887.08 μmol, 30.06 μL), cesium carbonate (867.09 mg, 2.66 mmol) were added to a stirred solution of N′—[(2,3-dichlorophenyl)methyl]-N′-(2-pyridylmethyl)oxamide (300 mg, 887.08 μmol), tert-butyl N-(7-bromo-2-tetrahydropyran-2-yl-pyrazolo[4,3-c]pyridin-4-yl)-N-tert-butoxycarbonyl-carbamate (441.22 mg, 887.08 μmol) and rac-(1R,2R)—N1,N2-dimethylcyclohexane-1,2-diamine (126.18 mg, 887.08 μmol) in 1,4-dioxane (15 mL) under Ar atmosphere and stirred at 100° C. for 12 hr in closed vial. The reaction mixture was filtered and evaporated in vacuo. Crude product was combined with material from a separate batch and dissolved in DCM (40 mL), washed with 10% aq. solution of ammonia. Organic phase dried over anhydrous sodium sulfate and evaporated to give tert-butyl N-tert-butoxycarbonyl-N-[7-[2-[(2,3-dichlorophenyl)methyl-(2-pyridylmethyl)amino]-2-oxo-acetyl]amino]-2-tetrahydropyran-2-yl-pyrazolo[4,3-c]pyridin-4-yl]carbamate (1.3 g, crude) as a brown oil.

LCMS (ESI): [M+H]⁺m/z: calcd 754.25; found 754.0; Rt=1.678 min.

### Step 5: The synthesis of N-(4-amino-2H-pyrazolo[4,3-c]pyridin-7-yl)-N′—[(2,3-dichlorophenyl)methyl]-N′-(2-pyridylmethyl)oxamide

Tert-butyl N-tert-butoxycarbonyl-N-[7-[2-[(2,3-dichlorophenyl)methyl-(2-pyridylmethyl)amino]-2-oxo-acetyl]amino]-2-tetrahydropyran-2-yl-pyrazolo[4,3-c]pyridin-4-yl]carbamate (1.3 g, 602.92 μmol) was dissolved in Dioxane (6 mL) saturated with HCl (10% by weight). MeOH (6 mL) was added for better solubility. The RM was stirred for 12 hr at 20° C. then concentrated in vacuo. The residue was dissolved in 4 mL of MeOH, filtered from insoluble impurities and subjected to HPLC (Mobile Phase, Column): SYSTEM 0.5-6.5 min 10-65% H₂O+FA/ACN flow 30 mL/min (loading pump 4 mL/min ACN), Column CROMATOREX Phenil C18 100×19 mm) to afford N-(4-amino-2H-pyrazolo[4,3-c]pyridin-7-yl)-N′—[(2,3-dichlorophenyl)methyl]-N′-(2-pyridylmethyl)oxamide (36.2 mg, 70.11 μmol, 11.63% yield, HCOOH) as a yellow solid.

¹H NMR (500 MHz, DMSO-d₆) δ 4.17-5.19 (m, 4H), 6.77 (s, 2H), 7.26-7.42 (m, 4H), 7.52-7.67 (m, 2H), 7.72-7.84 (m, 1H), 8.15-8.25 (m, 1H), 8.41-8.56 (m, 1H), 9.76-10.72 (m, 1H), 12.86 (s, 1H).

### Example 115. Synthesis of N-(4-amino-1H-pyrazolo[4,3-c]pyridin-7-yl)-N′-benzyl-N′-(3,3,3-trifluoropropyl)oxamide (Compound 102)

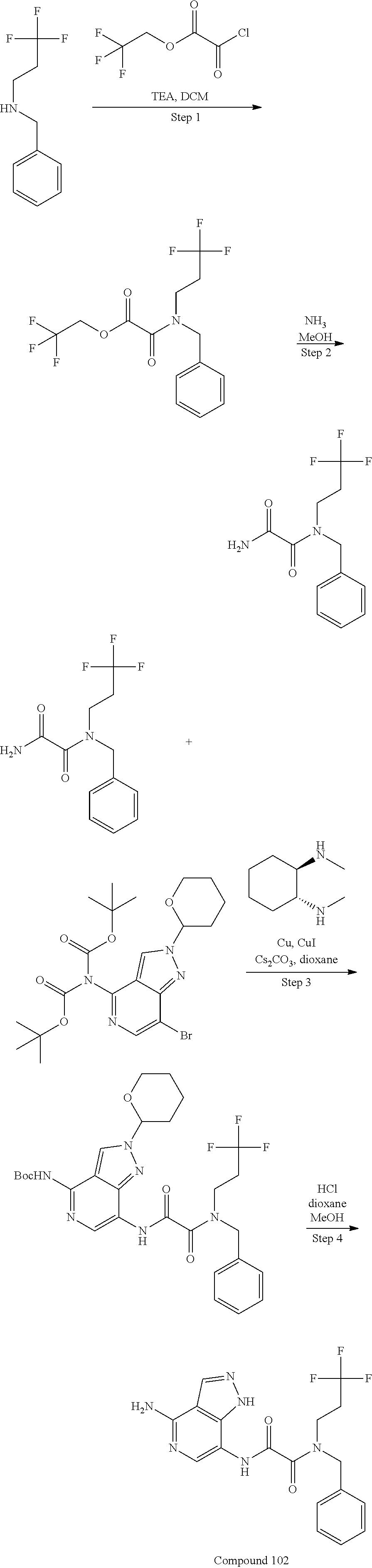

### Step 1:2,2,2-trifluoroethyl 2-[benzyl(3,3,3-trifluoropropyl)amino]-2-oxo-acetate

N-benzyl-3,3,3-trifluoro-propan-1-amine (1 g, 4.92 mmol) was dissolved in DCM (48.29 mL) and TEA (1.24 g, 12.30 mmol, 1.71 mL) was added. The reaction mixture was cooled and 2,2,2-trifluoroethyl 2-chloro-2-oxo-acetate (937.51 mg, 4.92 mmol) was added dropwise. After that it was stirred at rt overnight. Then water (10 mL) was added, organic layer was stirred with Brine and dried over anhydrous sodium sulfate evaporated in vacuum to afford 2,2,2-trifluoroethyl 2-[benzyl(3,3,3-trifluoropropyl)amino]-2-oxo-acetate (1.7 g, 4.76 mmol, 96.70% yield) as a yellow oil.

LCMS (ESI): [M+H]⁺m/z: calcd 358.09; found 358.0; Rt=1.491 min.

### Step 2: N′-benzyl-N′-(3,3,3-trifluoropropyl)oxamide

2,2,2-trifluoroethyl 2-[benzyl(3,3,3-trifluoropropyl)amino]-2-oxo-acetate (1.7 g, 4.76 mmol) was dissolved in NH3/MeOH (30 mL) and stirred overnight at rt. Then it was evaporated in vacuum and subjected to CC (CHCl₃-MeCN was used as an eluent mixture) to afford N′-benzyl-N′-(3,3,3-trifluoropropyl)oxamide (1 g, 3.65 mmol, 76.63% yield) as a light-yellow oil.

LCMS (ESI): [M+H]⁺m/z: calcd 275.1; found 275.0; Rt=1.159 min.

### Step 3: tert-butyl N-[7-[[2-[benzyl(3,3,3-trifluoropropyl)amino]-2-oxo-acetyl]amino]-2-tetrahydropyran-2-yl-pyrazolo[4,3-c]pyridin-4-yl]carbamate

A mixture of tert-butyl N-(7-bromo-2-tetrahydropyran-2-yl-pyrazolo[4,3-c]pyridin-4-yl)-N-tert-butoxycarbonyl-carbamate (598.52 mg, 1.20 mmol), N′-benzyl-N′-(3,3,3-trifluoropropyl)oxamide (0.3 g, 1.09 mmol), Cesium carbonate (534.64 mg, 1.64 mmol), rac-(1R,2R)—N1,N2-dimethylcyclohexane-1,2-diamine (124.48 mg, 875.15 μmol) and CuI (125.00 mg, 656.36 μmol, 22.24 μL) with a few mg of Cu(3.48 mg, 54.70 μmol) in dioxane (4.98 mL) was stirred in a sealed vial under argon at 105° C. for 48 hr. The resulting mixture was cooled down and filtered through silica gel to afford tert-butyl N-[7-[[2-[benzyl(3,3,3-trifluoropropyl)amino]-2-oxo-acetyl]amino]-2-tetrahydropyran-2-yl-pyrazolo[4,3-c]pyridin-4-yl]carbamate (0.6 g, 1.02 mmol, 92.87% yield) as a brown solid.

LCMS (ESI): [M−H]⁻ m/z: calcd 491.2; found 491.2; Rt=1.202 min.

### Step 4: The synthesis of N-(4-amino-1H-pyrazolo[4,3-c]pyridin-7-yl)-N′-benzyl-N′-(3,3,3-trifluoropropyl)oxamide

tert-butyl N-[7-[[2-[benzyl(3,3,3-trifluoropropyl)amino]-2-oxo-acetyl]amino]-2-tetrahydropyran-2-yl-pyrazolo[4,3-c]pyridin-4-yl]carbamate (0.6 g, 1.02 mmol) was dissolved in MeOH (10 mL) and diox/HCl (1.02 mmol, 5 mL) was added. Then mixture was stirred at rt overnight. The solution was evaporated and submitted to reverse phase HPLC (column: SunFire C18 100×19 mm, 5 μm; mobile phase: 20-45% 2-7.5 min water-MeCN+HCl, flow rate: 30 mL/min) to afford N-(4-amino-1H-pyrazolo[4,3-c]pyridin-7-yl)-N′-benzyl-N′-(3,3,3-trifluoropropyl)oxamide (65.4 mg, 0.16 mmol, 15.8% yield).

LCMS (ESI): [M+H]⁺m/z: calcd 407.16; found 407.2; Rt=1.193 min.

### Example 116. Synthesis of N1-(4-amino-1H-pyrazolo[4,3-c]pyridin-7-yl)-N2-(3-(dimethylamino)-2-methylbenzyl)-N2-(pyridin-2-ylmethyl)oxalamide (Compound 124)

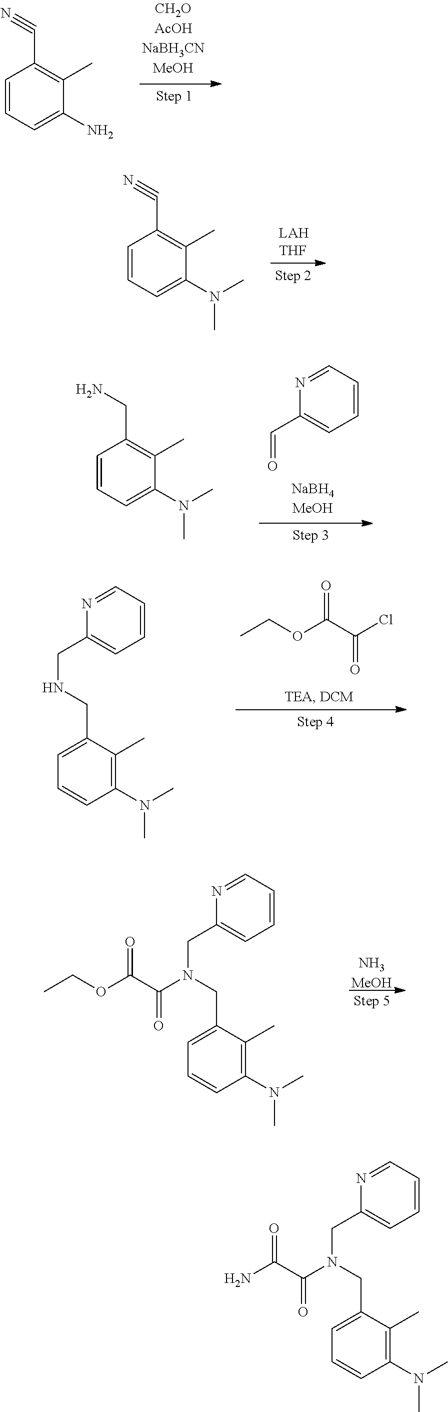

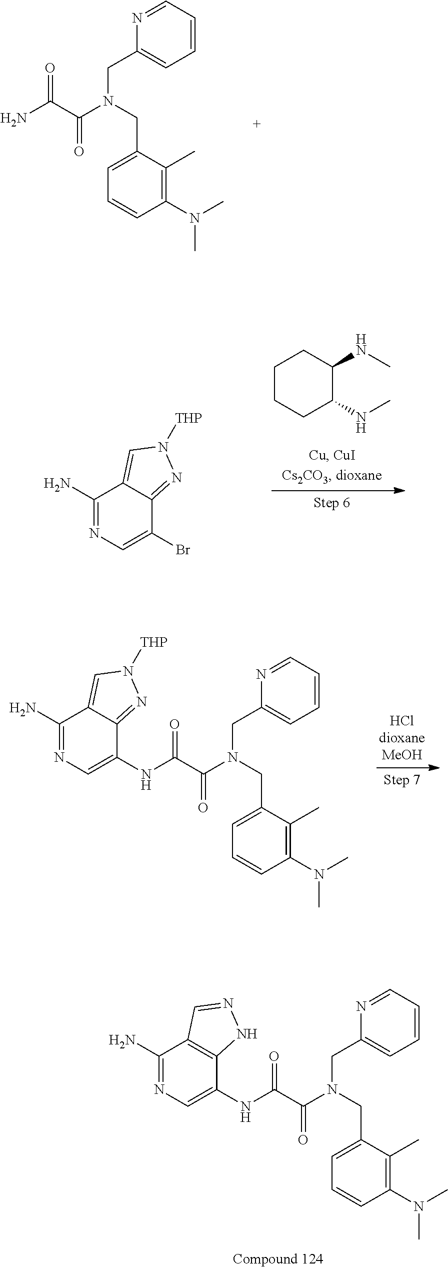

### Step 1:3-(dimethylamino)-2-methyl-benzonitrile

Formaldehyde, 37% w/w aq. soln., stab. with 7-8% methanol (12.90 g, 158.90 mmol, 11.91 mL, 37% purity) and acetic acid (12.72 g, 211.86 mmol, 12.13 mL) were added to a stirred solution of 3-amino-2-methyl-benzonitrile (7 g, 52.97 mmol) in methanol (206.87 mL) at 25° C. The resulting mixture was stirred at 25° C. for 1 hr, then Sodium cyanoborohydride (8.32 g, 132.41 mmol) was added in one portion at 25° C. (foaming!). The reaction mixture was stirred at 25° C. for 18 hr, and then concentrated in vacuo. The residue was diluted with 10% aqueous sodium hydroxide solution (50 mL) and extracted with dichloromethane (2×30 mL). The combined organic extracts were dried over anhydrous sodium sulfate and concentrated in vacuo to afford crude 3-(dimethylamino)-2-methyl-benzonitrile (6.5 g, 40.57 mmol, 76.60% yield) as a light-brown gum which was used directly in the next step.

LCMS (ESI): [M+H]⁺m/z: calcd 161.11; found 161.0; Rt=1.193 min.

### Step 2:3-(aminomethyl)-N,N,2-trimethyl-aniline

LiAlH₄ (355.34 mg, 9.36 mmol) was suspended in THF (25 mL) and solution of 3-(dimethylamino)-2-methyl-benzonitrile (1 g, 6.24 mmol) 3-(dimethylamino)-2-methyl-benzonitrile (1 g, 6.24 mmol) in THF (25 mL) was added dropwise to the previous suspension. After the addition was completed, the reaction mixture was stirred for 12 hr at 25° C. Water (10 mL) was carefully added dropwise to the precooled reaction mixture. The resulting mixture was stirred for 30 min and filtered. The filtercake was rinsed with THF (3×40 mL) and the filtrate was concentrated in a vacuum to obtain crude 3-(aminomethyl)-N,N,2-trimethyl-aniline (0.9 g, 5.48 mmol, 87.79% yield) as a yellow oil which was used in the next step without purification.

LCMS (ESI): [M+H]⁺m/z: calcd 165.14; found 165.2; Rt=0.188 min.

### Step 3: N,N,2-trimethyl-3-[(2-pyridylmethylamino)methyl]aniline

Pyridine-2-carbaldehyde (586.91 mg, 5.48 mmol, 522.17 μL) was added to a stirred solution of 3-(aminomethyl)-N,N,2-trimethyl-aniline (0.9 g, 5.48 mmol) in Methanol (9.99 mL) and was stirred at 20° C. for 10 hr. Then Sodium Borohydride (207.29 mg, 5.48 mmol, 193.01 μL) was added, the reaction mixture was stirred for 2 hr additional. The reaction mixture was evaporated in vacuo. The residue was dissolved in DCM (50 mL) and washed with water (2×15 mL). The organic layer was dried over anhydrous sodium sulfate and filtered. DCM was evaporated under reduce pressure to give N,N,2-trimethyl-3-[(2-pyridylmethylamino)methyl]aniline as a yellow oil.

LCMS (ESI): [M+H]⁺m/z: calcd 256.18; found 256.2; Rt=0.653 min.

### Step 4: ethyl 2-[[3-(dimethylamino)-2-methyl-phenyl]methyl-(2-pyridylmethyl)amino]-2-oxo-acetate

N,N,2-trimethyl-3-[(2-pyridylmethylamino)methyl]aniline (800.00 mg, 3.13 mmol) and TEA (317.01 mg, 3.13 mmol, 436.66 μL) were dissolved in DCM (20 mL), cooled with ice-water bath and then ethyl 2-chloro-2-oxo-acetate (427.74 mg, 3.13 mmol, 350.03 μL) in 20 mL of DCM was added dropwise in 10 min. The solution was stirred overnight. The reaction mixture was washed with an aqueous solution of NaHCO₃, dried over anhydrous sodium sulfate and evaporated to give ethyl 2-[[3-(dimethylamino)-2-methyl-phenyl]methyl-(2-pyridylmethyl)amino]-2-oxo-acetate (0.98 g, 2.76 mmol, 88.01% yield) as a yellow oil.

LCMS (ESI): [M+H]⁺m/z: calcd 356.2; found 356.2; Rt=0.744 min.

### Step 5: N′-[[3-(dimethylamino)-2-methyl-phenyl]methyl]-N′-(2-pyridylmethyl)oxamide

Ethyl 2-[3-(dimethylamino)-2-methyl-phenyl]methyl-(2-pyridylmethyl)amino]-2-oxo-acetate (0.98 g, 2.76 mmol) was dissolved in NH3/methanol (50 mL) solution (10% by weight) and stirred overnight, then concentrated in vacuo to give N′-[[3-(dimethylamino)-2-methyl-phenyl]methyl]-N′-(2-pyridylmethyl)oxamide (0.78 g, 2.39 mmol, 86.67% yield) as a yellow oil.

LCMS (ESI): [M+H]⁺m/z: calcd 327.18; found 327.0; Rt=0.635 min.

### Step 6: N-(4-amino-2-tetrahydropyran-2-yl-pyrazolo[4,3-c]pyridin-7-yl)-N′-[[3-(dimethylamino)-2-methyl-phenyl]methyl]-N′-(2-pyridylmethyl)oxamide

7-bromo-2-tetrahydropyran-2-yl-pyrazolo[4,3-c]pyridin-4-amine (300.00 mg, 1.01 mmol), N′-[3-(dimethylamino)-2-methyl-phenyl]methyl]-N′-(2-pyridylmethyl)oxamide (0.25 g, 765.95 μmol), Copper (I) iodide (38.46 mg, 201.92 μmol, 6.84 μL), Cesium carbonate (438.59 mg, 1.35 mmol) and (1R,2R)—N1,N2-dimethylcyclohexane-1,2-diamine (43.08 mg, 302.88 μmol) were mixed in Dioxane (4 mL) under argon, and then stirred overnight at 95° C. for 12 hr in vial. The reaction mixture was filtered, then evaporated. The residue was dissolved in 15 mL of DCM washed with an aqueous ammonium solution, dried over anhydrous sodium sulfate and evaporated to give crude N-(4-amino-2-tetrahydropyran-2-yl-pyrazolo[4,3-c]pyridin-7-yl)-N′-[[3-(dimethylamino)-2-methyl-phenyl]methyl]-N′-(2-pyridylmethyl)oxamide (0.33 g, 608.15 μmol, 90.36% yield).

LCMS (ESI): [M+H]⁺m/z: calcd 543.29; found 543.2; Rt=0.859 min.

### Step 7: The synthesis of N1-(4-amino-1H-pyrazolo[4,3-c]pyridin-7-yl)-N2-(3-(dimethylamino)-2-methylbenzyl)-N2-(pyridin-2-ylmethyl)oxalamide

N-(4-amino-2-tetrahydropyran-2-yl-pyrazolo[4,3-c]pyridin-7-yl)-N′-[[3-(dimethylamino)-2-methyl-phenyl]methyl]-N′-(2-pyridylmethyl)oxamide (0.35 g, 645.01 μmol) was dissolved in MeOH (1 mL) and dioxane/HCl (12.90 mmol, 1 mL) was added thereto. Then it was stirred at rt for 2 hr. The reaction mixture was evaporated. The crude product was purified by HPLC (Device (Mobile Phase, Column): SYSTEM 0-2-10 min 30-75% H₂O/MeOH, flow 30 mL/min ((loading pump 4 mL MeOH) target mass 328, column: Chromatorex C18 SMB100-5T 100×19 mm, 5 microM) to afford N1-(4-amino-1H-pyrazolo[4,3-c]pyridin-7-yl)-N2-(3-(dimethylamino)-2-methylbenzyl)-N2-(pyridin-2-ylmethyl)oxalamide (0.04 g, 87.3 μmol, 13.5% yield) as an orange solid.

¹H NMR (500 MHz, DMSO-d₆) & 2.07-2.15 (m, 3H), 2.54-2.58 (m, 6H), 4.50-4.62 (m, 2H), 4.78-4.93 (m, 2H), 6.65 (s, 2H), 6.85-6.95 (m, 1H), 6.97-7.03 (m, 1H), 7.09-7.17 (m, 1H), 7.22-7.30 (m, 1H), 7.33-7.44 (m, 1H), 7.55-7.66 (m, 1H), 7.73-7.80 (m, 1H), 8.13-8.19 (m, 1H), 8.47-8.52 (m, 1H), 10.53 (s, 1H), 12.20-13.62 (m, 1H).

LCMS (ESI): [M+H]⁺m/z: calcd 459.25; found 459.2; Rt=0.563 min.

### Example 117. Synthesis of N-(4-amino-1H-pyrazolo[4,3-c]pyridin-7-yl)-N′-isopropyl-N′-(0-tolylmethyl)oxamide (Compound 133)

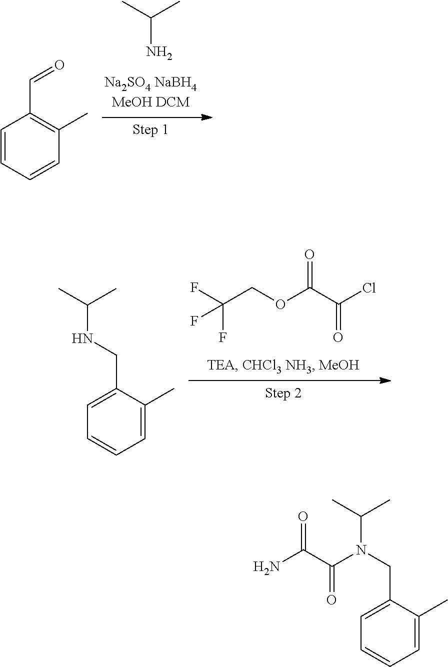

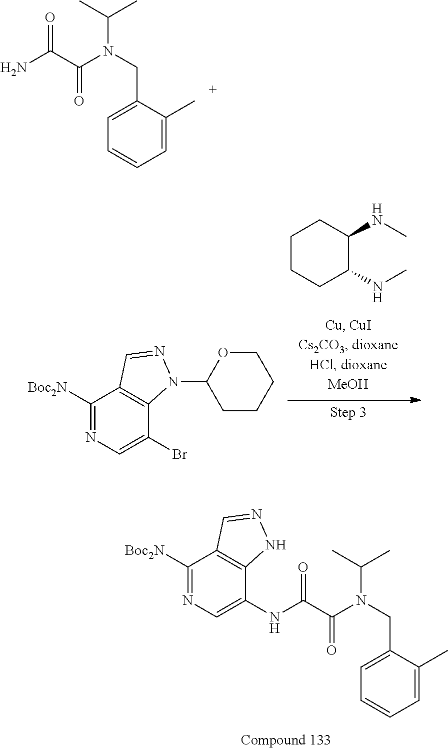

### Step 1: N-(o-tolylmethyl)propan-2-amine

To a stirred solution of 2-methylbenzaldehyde (0.3 g, 2.50 mmol, 288.74 μL) and propan-2-amine (172.75 mg, 2.92 mmol, 0.25 mL) in DCM (10 mL) was added Sodium Sulfate (3.5 g, 24.64 mmol, 1.31 mL). The resulting reaction mixture was stirred at 25° C. for 16 hr. Upon completion, the reaction mixture was filtered, the filtrate was quenched with water (20 mL). The aqueous phase was extracted with CHCl₃ (20 mL). The combined organic phase was dried over anhydrous sodium sulfate and concentrated under reduced pressure. To a stirred solution of residue in MeOH (10 mL) was added Sodium Borohydride (0.45 g, 11.90 mmol, 418.99 μL). The resulting reaction mixture was stirred at 25° C. for 16 hr. Upon completion, the reaction mixture was evaporated, quenched with water (20 mL). The aqueous phase was extracted with CHCl₃ (2×20 mL). The combined organic phase was dried over anhydrous sodium sulfate and concentrated under reduced pressure to afford N-(o-tolylmethyl)propan-2-amine (0.4 g, 2.45 mmol, 98.13% yield) as a white solid.

LCMS (ESI): [M+H]⁺m/z: calcd 164.15; found 164.2; Rt=0.781 min.

### Step 2: N′-isopropyl-N′-(o-tolylmethyl)oxamide

To a solution of N-(o-tolylmethyl)propan-2-amine (0.4 g, 2.45 mmol) and Triethylamine (326.70 mg, 3.23 mmol, 0.45 mL) in CHCl₃ (10 mL) was added 2,2,2-trifluoroethyl 2-chloro-2-oxo-acetate (0.5 g, 2.62 mmol) at 25° C. The resulting reaction mixture was allowed to warm to room temperature and stirred for 16 hr at 25° C. After 16 hr the reaction mixture was quenched with water (20 mL) and extracted with CHCl₃ (2×20 mL). The organic layer was dried over anhydrous sodium sulfate and evaporated under reduced pressure. To a stirred solution of residue in MeOH (5 mL) was added MeOH/NH3 (5 mL). The resulting reaction mixture was stirred at 25° C. for 3 hr. Upon completion, the reaction mixture was concentrated under reduced pressure to afford N′-isopropyl-N′-(o-tolylmethyl)oxamide (0.55 g, 2.35 mmol, 95.81% yield) as a yellow solid.

LCMS (ESI): [M+H]⁺m/z: calcd 235.15; found 235.0; Rt=1.042 min.

### Step 3: The synthesis of N-(4-amino-1H-pyrazolo[4,3-c]pyridin-7-yl)-N′-isopropyl-N′-(0-tolylmethyl)oxamide

N′-isopropyl-N′-(o-tolylmethyl)oxamide (0.1 g, 426.82 μmol), tert-butyl N-(7-bromo-2-tetrahydropyran-2-yl-pyrazolo[4,3-c]pyridin-4-yl)-N-tert-butoxycarbonyl-carbamate (212.29 mg, 426.82 μmol), Copper (20 mg, 314.73 μmol), CuI (80 mg, 420.06 μmol, 14.23 μL) and Cesium carbonate (0.3 g, 920.76 μmol) were mixed together in Dioxane (5 mL). The resulting suspension was degassed with argon at 25° C. for 0.1 hr. rac-(1R,2R)—N1,N2-dimethylcyclohexane-1,2-diamine (90.00 mg, 632.72 μmol, 0.1 mL) was added thereto and the resulting mixture was stirred for 24 hr at 100° C. Upon completion, the reaction mixture was concentrated under reduced pressure, dissolved in CHCl₃, dried over anhydrous sodium sulfate, filtered through a pad of silica gel and the filtrate was evaporated in vacuo. To a stirred solution of residue in MeOH (2 mL) was added Dioxane/HCl (2 mL). The resulting reaction mixture was stirred at 25° C. for 16 hr. Upon completion, the reaction mixture was concentrated under reduced pressure. The obtained crude product was purified by reverse phase HPLC chromatography (Device (Mobile Phase, Column): SYSTEM 20-20-70% 0-1.3-5.3 min H₂O/MeOH/0.1% NH₄OH, flow: 30 mL/min (loading pump 4 mL/min MeOH) target mass 366, 268 column: XBridge BEH C18 5 μm 130A) to afford product N-(4-amino-1H-pyrazolo[4,3-c]pyridin-7-yl)-N′-isopropyl-N′-(o-tolylmethyl)oxamide (11 mg, 30.02 μmol, 7.03% yield) as a white solid.

¹H NMR (600 MHz, CD₃OD) δ 0.89-1.34 (m, 6H), 2.17-2.45 (m, 3H), 4.48-4.60 (m, 1H), 4.60-4.80 (m, 2H), 6.98-7.40 (m, 4H), 7.78-8.48 (m, 2H).

LCMS (ESI): [M+H]⁺m/z: calcd 367.21; found 367.2; Rt=2.388 min.

### Example 118. Synthesis of N′-[(3-aminophenyl)methyl]-N-(4-amino-1H-pyrazolo[4,3-c]pyridin-7-yl)-N′-benzyl-oxamide (Compound 145)

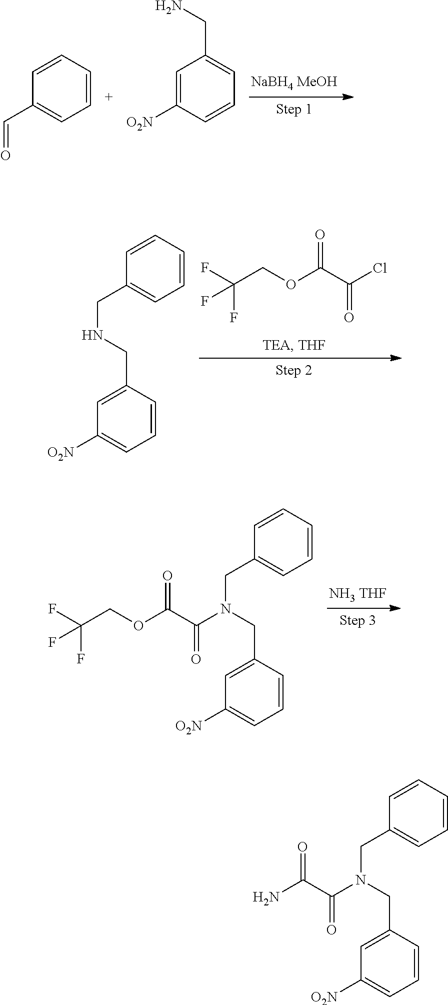

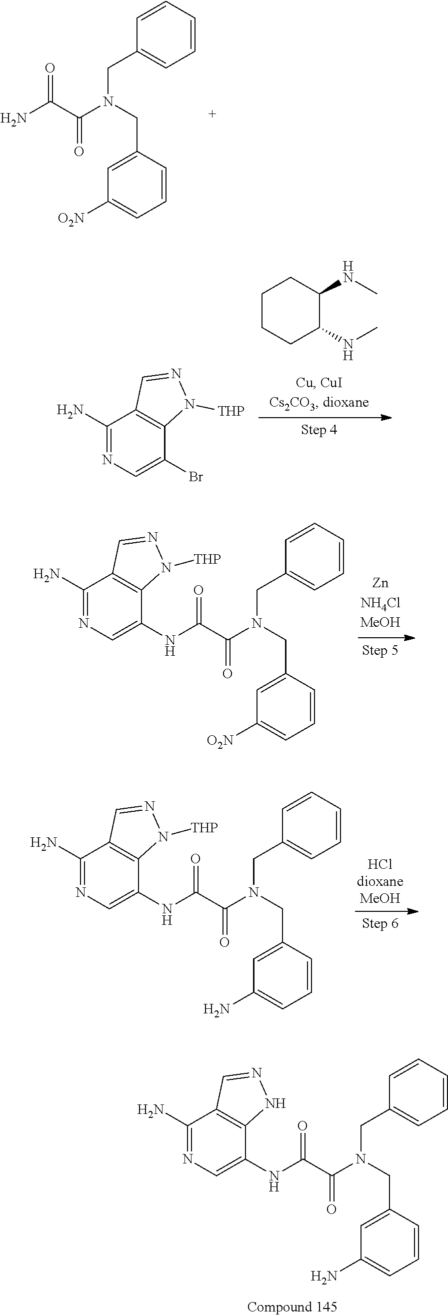

### Step 1: N-[(3-nitrophenyl)methyl]-1-phenyl-methanamine

A solution of benzaldehyde (1 g, 9.42 mmol) and (3-nitrophenyl)methanamine (1.43 g, 9.42 mmol) in MeOH (20.00 mL) was stirred at 20° C. for 12 hr. To this solution, Sodium Borohydride (392.13 mg, 10.37 mmol, 365.11 μL) was added and the resulting mixture was stirred for 12 hr. The solvent was removed in vacuo, the residue was taken up with water (20 mL) and extracted with DCM (3×20 mL). The combined organic layer was washed with brine (10 mL), dried over anhydrous sodium sulfate and evaporated to obtain N-[(3-nitrophenyl)methyl]-1-phenyl-methanamine (1.5 g, 6.19 mmol, 65.70% yield) as a yellow oil.

LCMS (ESI): [M+H]⁺m/z: calcd 243.12; found 243.2; Rt=1.537 min.

### Step 2:2,2,2-trifluoroethyl 2-[benzyl-[(3-nitrophenyl)methyl]amino]-2-oxo-acetate

To a solution of N-[(3-nitrophenyl)methyl]-1-phenyl-methanamine (1.5 g, 6.19 mmol) and TEA (939.76 mg, 9.29 mmol, 1.29 mL) in THF (25.51 mL) was added 2,2,2-trifluoroethyl 2-chloro-2-oxo-acetate (1.42 g, 7.43 mmol) dropwise at 0° C. under argon. The reaction mixture was then stirred for 12 hr at r.t., then evaporated in vacuo to give 2,2,2-trifluoroethyl 2-[benzyl-[(3-nitrophenyl)methyl]amino]-2-oxo-acetate (2.1 g, 5.30 mmol, 85.58% yield) as a yellow oil.

LCMS (ESI): [M+H]⁺m/z: calcd 397.1; found 397.2; Rt=3.760 min.

### Step 3: N′-benzyl-N′-[(3-nitrophenyl)methyl]oxamide

2,2,2-trifluoroethyl 2-[benzyl-[(3-nitrophenyl)methyl]amino]-2-oxo-acetate (2.1 g, 5.30 mmol) was dissolved in THF (20 mL) and was blow ammonium (2.80 g, 164.33 mmol). The resulting solution was stirred at 0° C. for 14 hr. The resulting mixture was evaporated in vacuo and residue was triturated with THF (2×20 mL), filtered and combined organic layers were evaporated in vacuo to leave 1.5 g of crude product which was purification by column chromatography on silica gel using CHCl₃/CH₃CN gradient (10-100% CH₃CN) to afford N′-benzyl-N′-[(3-nitrophenyl)methyl]oxamide (0.7 g, 2.23 mmol, 42.16% yield) as a yellow oil.

LCMS (ESI): [M−H]⁻ m/z: calcd 312.1; found 312.0; Rt=1.215 min.

### Step 4: N-(4-amino-1-tetrahydropyran-2-yl-pyrazolo[4,3-c]pyridin-7-yl)-N′-benzyl-N′-[(3-nitrophenyl)methyl]oxamide

7-bromo-1-tetrahydropyran-2-yl-pyrazolo[4,3-c]pyridin-4-amine (341.44 mg, 1.15 mmol), N′-benzyl-N′-[(3-nitrophenyl)methyl]oxamide (0.3 g, 957.53 μmol), Cu(3.04 mg, 47.88 μmol), CuI (0.15 g, 787.61 μmol, 26.69 μL), cesium carbonate (467.97 mg, 1.44 mmol) and

(1R,2R)—N1,N2-dimethylcyclohexane-1,2-diamine (163.44 mg, 1.15 mmol) were mixed in dioxane (6.00 mL), purged with Ar for 15 minutes and then heated in the sealed tube at 110° C. for 48 hr. Final mixture was filtered and dioxane was evaporated in vacuo to afford N-(4-amino-1-tetrahydropyran-2-yl-pyrazolo[4,3-c]pyridin-7-yl)-N′-benzyl-N′-[(3-nitrophenyl)methyl]oxamide (0.6 g, crude) as a black gum.

LCMS (ESI): [M+H]⁺m/z: calcd 530.22; found 530.0; Rt=3.316 min.

### Step 5: N′-[(3-aminophenyl)methyl]-N-(4-amino-1-tetrahydropyran-2-yl-pyrazolo[4,3-c]pyridin-7-yl)-N′-benzyl-oxamide

To a solution of N-(4-amino-1-tetrahydropyran-2-yl-pyrazolo[4,3-c]pyridin-7-yl)-N′-benzyl-N′-[(3-nitrophenyl)methyl]oxamide (0.6 g, 1.13 mmol) and Ammonium Chloride (969.73 mg, 18.13 mmol, 633.81 μL) in MeOH (9.47 mL) was added zinc (370.45 mg, 5.67 mmol) at 0° C. under argon. The reaction mixture was then stirred for 12 hr at r.t., the solid was filtered and the filtrate was evaporated under vacuo and poured in water (20 mL) and extracted with EtOAc (20 mL). The combined organic extracts dried over anhydrous sodium sulfate and evaporated in vacuo to afford N′-[(3-aminophenyl)methyl]-N-(4-amino-1-tetrahydropyran-2-yl-pyrazolo[4,3-c]pyridin-7-yl)-N′-benzyl-oxamide (0.4 g, 800.70 μmol, 70.67% yield) as a brown solid.

### Step 6: The synthesis of N′-[(3-aminophenyl)methyl]-N-(4-amino-1H-pyrazolo[4,3-c]pyridin-7-yl)-N′-benzyl-oxamide

Hydrogen chloride, 4M in 1,4-dioxane, 99% (4.02 g, 110.37 mmol, 5.03 mL) was added to a solution of N′-[(3-aminophenyl)methyl]-N-(4-amino-1-tetrahydropyran-2-yl-pyrazolo[4,3-c]pyridin-7-yl)-N′-benzyl-oxamide (0.29 g, 580.51 μmol) in MeOH (13.99 mL). The reaction mixture was stirred at 20° C. for 12 hr, then evaporated was purified by RP-HPLC (column: XBridge BEH18 SMB100-BT 100×19 mm; 0-0-25% 0-2-5 min H₂O/CH₃OH/0.1% FA, flow: 30 mL/min) to give N′-[(3-aminophenyl)methyl]-N-(4-amino-1H-pyrazolo[4,3-c]pyridin-7-yl)-N′-benzyl-oxamide (109.60 mg, 215.96 μmol, 37.20% yield, 2HCOOH) as a light-yellow solid.

¹H NMR (500 MHz, DMSO-d₆) δ 4.40-4.53 (m, 2H), 4.56-4.74 (m, 2H), 7.15-7.39 (m, 10H), 7.85-8.17 (m, 1H), 8.30-10.57 (m, 4H), 10.93-11.35 (m, 1H), 11.97-14.91 (m, 1H).

LCMS (ESI): [M+H]⁺m/z: calcd 416.2; found 416.2; Rt=1.961 min.

### Example 119. The synthesis of N′-[(3-acetamidophenyl)methyl]-N-(4-amino-1H-pyrazolo[4,3-c]pyridin-7-yl)-N′-benzyl-oxamide (Compound 161)

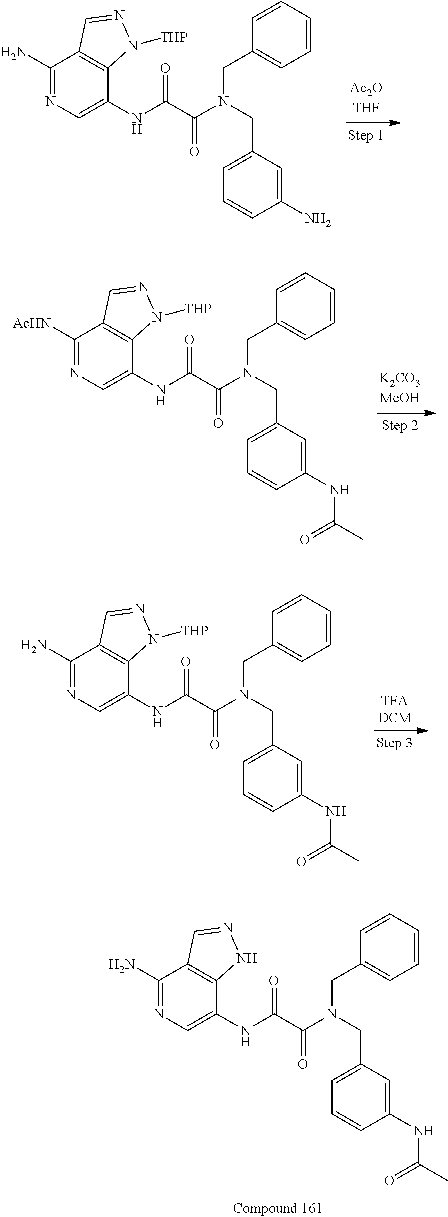

### Step 1: N′-[(3-acetamidophenyl)methyl]-N-(4-acetamido-1-tetrahydropyran-2-yl-pyrazolo[4,3-c]pyridin-7-yl)-N′-benzyl-oxamide

To a solution of N′-[(3-aminophenyl)methyl]-N-(4-amino-1-tetrahydropyran-2-yl-pyrazolo[4,3-c]pyridin-7-yl)-N′-benzyl-oxamide (0.4 g, 800.70 μmol) in THF (20 mL) was added Acetic anhydride (122.61 mg, 1.20 mmol, 113.32 μL) dropwise at 0° C. under argon. The reaction mixture was then stirred for 12 hr at r.t., then evaporated in vacuo to give N′-[(3-acetamidophenyl)methyl]-N-(4-acetamido-1-tetrahydropyran-2-yl-pyrazolo[4,3-c]pyridin-7-yl)-N′-benzyl-oxamide (0.4 g, 685.36 μmol, 85.59% yield) as a yellow oil.

LCMS (ESI): [M+H]⁺m/z: calcd 584.26; found 584.2; Rt=2.951 min.

### Step 2: N′-[(3-acetamidophenyl)methyl]-N-(4-amino-1-tetrahydropyran-2-yl-pyrazolo[4,3-c]pyridin-7-yl)-N′-benzyl-oxamide

A solution of N′-[(3-acetamidophenyl)methyl]-N-(4-acetamido-1-tetrahydropyran-2-yl-pyrazolo[4,3-c]pyridin-7-yl)-N′-benzyl-oxamide (0.4 g, 685.36 μmol) and Potassium carbonate, anhydrous, 99% (189.44 mg, 1.37 mmol, 82.73 μL) in MeOH (30 mL) was stirred at 70° C. for 12 hr. The solvent was removed in vacuo, the residue was taken up with water (20 mL) and extracted with DCM (3×20 mL). The combined organic layers were washed with brine (10 mL), dried over anhydrous sodium sulfate and evaporated to obtain N′-[(3-acetamidophenyl)methyl]—N-(4-amino-1-tetrahydropyran-2-yl-pyrazolo[4,3-c]pyridin-7-yl)-N′-benzyl-oxamide (0.25 g, 461.60 μmol, 67.35% yield) as a light-yellow solid.

### Step 3: The synthesis of N′-[(3-acetamidophenyl)methyl]-N-(4-amino-1H-pyrazolo[4,3-c]pyridin-7-yl)-N′-benzyl-oxamide

Trifluoroacetic acid (15 g, 131.55 mmol, 10.14 mL) was added to a solution of N′-[(3-acetamidophenyl)methyl]-N-(4-amino-1-tetrahydropyran-2-yl-pyrazolo[4,3-c]pyridin-7-yl)-N′-benzyl-oxamide (0.25 g, 461.60 μmol) in DCM (4.91 mL). The reaction mixture was stirred at 20° C. for 12 hr, then evaporated was purified by RP-HPLC (column: XBridge BEH18 SMB100-BT 100×19 mm; 20-70% 0-5 min H₂O/CH₃OH/0.1% NH₄OH, flow: 30 mL/min) to give N′-[(3-acetamidophenyl)methyl]-N-(4-amino-1H-pyrazolo[4,3-c]pyridin-7-yl)-N′-benzyl-oxamide (0.0096 g, 20.98 μmol, 4.55% yield) as a light-yellow solid.

¹H NMR (500 MHz, DMSO-d₆) δ 1.93-2.10 (m, 3H), 4.35-4.47 (m, 2H), 4.57-4.77 (m, 2H), 6.13-6.68 (m, 2H), 6.86-7.13 (m, 2H), 7.14-7.42 (m, 7H), 7.42-7.53 (m, 1H), 7.54-7.62 (m, 1H), 7.63-7.74 (m, 1H), 8.07-8.21 (m, 1H), 9.98 (s, 1H).

LCMS (ESI): [M+H]⁺m/z: calcd 458.1; found 458.2; Rt=2.013 min.

### Example 120. The synthesis of N-(4-amino-1H-pyrazolo[4,3-c]pyridin-7-yl)-N′-benzyl-N′-[[3-(methylamino)phenyl]methyl]oxamide (Compound 93)

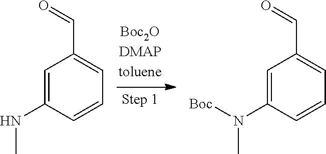

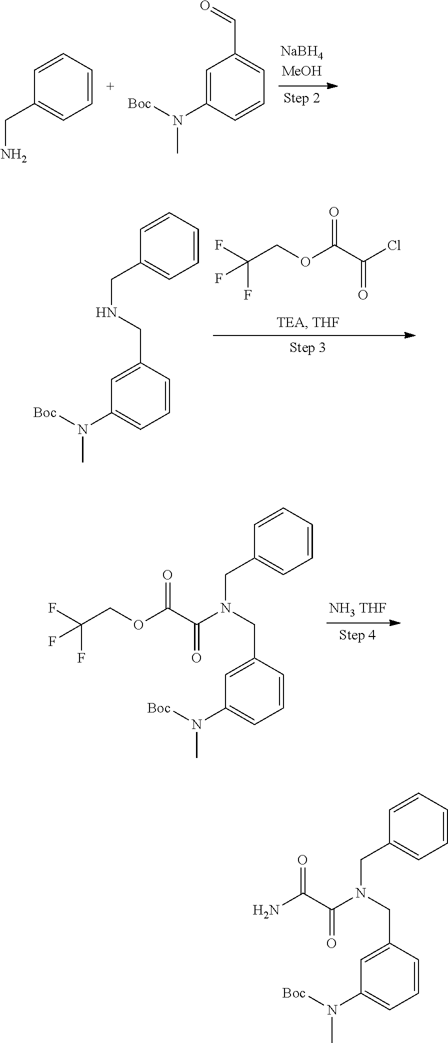

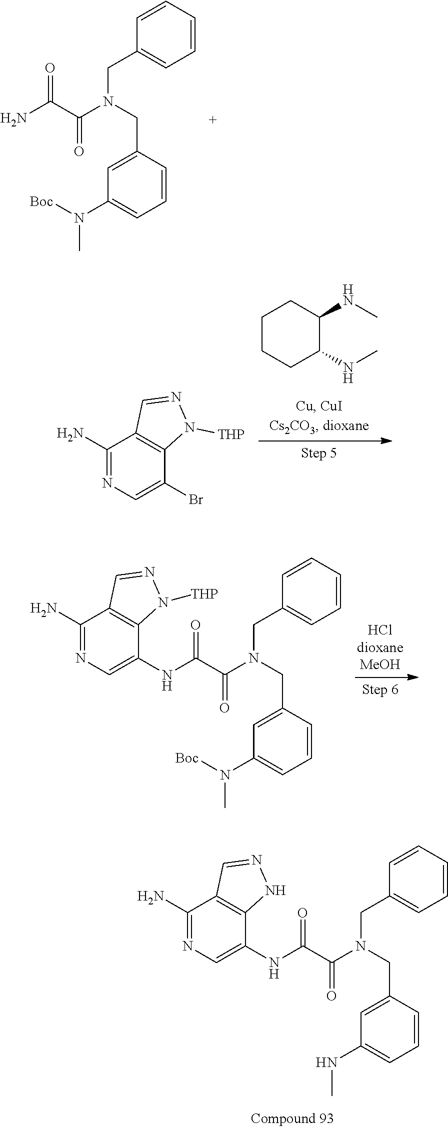

### Step 1: tert-butyl N-(3-formylphenyl)-N-methyl-carbamate

Di tert butyl dicarbonate (968.83 mg, 4.44 mmol, 1.02 mL) was added in one portion at 80° C. to a stirred slurry of 3-(methylamino)benzaldehyde (0.5 g, 3.70 mmol) and DMAP(45.19 mg, 369.92 μmol) in toluene (20 mL). The resulting mixture was stirred at 80° C. for 17 hr to form clear solution. The reaction mixture was transferred to a separatory funnel, washed with water (2×50 mL), dried over anhydrous sodium sulfate and concentrated in vacuo to afford tert-butyl N-(3-formylphenyl)-N-methyl-carbamate (0.6 g, 2.55 mmol, 68.94% yield) as a yellow oil.

### Step 2: tert-butyl N-[3-[(benzylamino)methyl]phenyl]-N-methyl-carbamate

A solution of tert-butyl N-(3-formylphenyl)-N-methyl-carbamate (0.6 g, 2.55 mmol) and phenylmethanamine (273.26 mg, 2.55 mmol) in MeOH (20 mL) was stirred at 20° C. for 12 hr. To this solution, Sodium Borohydride (106.12 mg, 2.81 mmol, 98.81 μL) was added and the resulting mixture was stirred for 12 hr. The solvent was removed in vacuo, the residue was taken up with water (20 mL) and extracted with DCM (3×20 mL). The combined organic layer was washed with brine (10 mL), dried over anhydrous sodium sulfate and evaporated to obtain tert-butyl N-[3-[(benzylamino)methyl]phenyl]-N-methyl-carbamate (0.8 g, 2.45 mmol, 96.10% yield) as a yellow oil.

LCMS (ESI): [M+H]⁺m/z: calcd 327.21; found 327.4; Rt=2.508 min.

### Step 3:2,2,2-trifluoroethyl 2-[benzyl-[[3-[tert-butoxycarbonyl(methyl)amino]phenylmethyl]amino]-2-oxo-acetate

To a solution of tert-butyl N-[3-[(benzylamino)methyl]phenyl]-N-methyl-carbamate (0.8 g, 2.45 mmol) and TEA (371.99 mg, 3.68 mmol, 512.38 μL) in THF (26.29 mL) was added 2,2,2-trifluoroethyl 2-chloro-2-oxo-acetate (560.25 mg, 2.94 mmol) dropwise at 0° C. under argon. The reaction mixture was then stirred for 12 hr at r.t., then evaporated in vacuo to give 2,2,2-trifluoroethyl 2-[benzyl-[[3-[tert-butoxycarbonyl(methyl)amino]phenyl]methyl]amino]-2-oxo-acetate (1 g, 2.08 mmol, 84.92% yield) as a yellow oil.

LCMS (ESI): [M+H]⁺m/z: calcd 458.2; found 458.4; Rt=3.002 min.

### Step 4: tert-butyl N-[3-[[benzyl(oxamoyl)amino]methyl]phenyl]-N-methyl-carbamate

2,2,2-trifluoroethyl 2-[benzyl-[[3-[tert-butoxycarbonyl(methyl)amino]phenyl]methyl]amino]-2-oxo-acetate (1 g, 2.08 mmol) was dissolved in THF (20 mL) and was blow ammonium (750.92 mg, 44.09 mmol). Resulting solution was stirred at 0° C. for 14 hr. The resulting mixture was evaporated in vacuo and residue was triturated with THF (2×20 mL), filtered and combined organic was evaporated in vacuo to leave 0.7 g of crude product which was purified by column chromatography on silica gel using CHCl₃/CH₃CN gradient (10-100% CH₃CN) to afford tert-butyl N-[3-[[benzyl(oxamoyl)amino]methyl]phenyl]-N-methyl-carbamate (0.25 g, 628.98 μmol, 30.22% yield) as a yellow oil.

LCMS (ESI): [M−H]⁻ m/z: calcd 396.19; found 396.2; Rt=1.347 min.

### Step 5: tert-butyl N-[3-[[[2-[(4-amino-1-tetrahydropyran-2-yl-pyrazolo[4,3-c]pyridin-7-yl)amino]-2-oxo-acetyl]-benzyl-amino methyl]phenyl]-N-methyl-carbamate

7-bromo-1-tetrahydropyran-2-yl-pyrazolo[4,3-c]pyridin-4-amine (242.97 mg, 817.68 μmol), tert-butyl N-[3-[[benzyl(oxamoyl)amino]methyl]phenyl]-N-methyl-carbamate (0.25 g, 628.98 μmol), Cu(0.01 g, 157.37 μmol), CuI (0.15 g, 787.61 μmol, 26.69 μL), cesium carbonate (307.40 mg, 943.48 μmol) and (1R,2R)—N1,N2-dimethylcyclohexane-1,2-diamine (0.15 g, 1.05 mmol) were mixed in dioxane (6 mL), purged with Ar for 15 minutes and then heated in the sealed tube at 105° C. for 48 hr. Final mixture was filtered and dioxane was evaporated in vacuo. The crude product 0.5 g was purified by RP-HPLC (column: XBridge C18 5 μm 130A; 45-45-55% 0-1.5-5 min H₂O/CH₃CN/0.1% NH₄OH, flow: 30 mL/min) to give tert-butyl N-[3-[[2-[(4-amino-1-tetrahydropyran-2-yl-pyrazolo[4,3-c]pyridin-7-yl)amino]-2-oxo-acetyl]-benzyl-amino]methyl]phenyl]-N-methyl-carbamate (0.1045 g, 170.28 μmol, 27.07% yield). as a brown solid.

LCMS (ESI): [M+H]⁺m/z: calcd 614.35; found 614.2; Rt=3.606 min.

### Step 6: The synthesis of N-(4-amino-1H-pyrazolo[4,3-c]pyridin-7-yl)-N′-benzyl-N′-[[3-(methylamino)phenyl]methyl]oxamide (Compound 93)

Hydrogen chloride solution 4.0M in dioxane (1.60 g, 43.88 mmol, 2 mL) was added to a solution of tert-butyl N-[3-[[2-[(4-amino-1-tetrahydropyran-2-yl-pyrazolo[4,3-c]pyridin-7-yl)amino]-2-oxo-acetyl]-benzyl-amino]methyl]phenyl]-N-methyl-carbamate (104.67 mg, 170.55 μmol) in MeOH (5 mL). The reaction mixture was stirred at 20° C. for 24 hr, then evaporated was purified by RP-HPLC (column: XBridge BEH18 SMB100-BT 100×19 mm; Oct. 10, 2040% 0-1.5-5 min H₂O/CH₃CN/0.1% NH₄OH, flow: 30 mL/min) to give N-(4-amino-1H-pyrazolo[4,3-c]pyridin-7-yl)-N′-benzyl-N′-[3-(methylamino)phenyl]methyl]oxamide (0.0121 g, 28.17 μmol, 16.52% yield) as a light-yellow solid.

LCMS (ESI): [M+H]⁺m/z: calcd 430.22; found 430.2; Rt=2.372 min.

### Example 121. Synthesis of N-(4-Amino-1HI-pyrazolo[4,3-c]pyridin-7-yl)-N′-benzyl-N′-chroman-4-yl-oxamide (Compound 97)

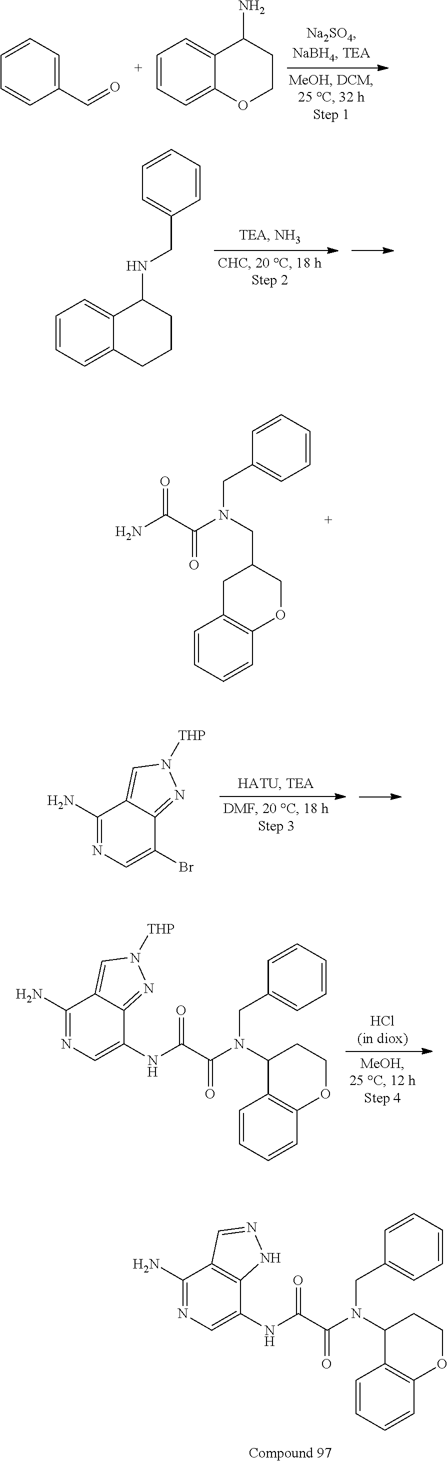

### Step 1: The Synthesis of N-Benzylchroman-4-amine

To a stirred solution of benzaldehyde (0.3 g, 2.83 mmol) and chroman-4-amine (0.53 g, 2.85 mmol, HCl) in DCM (10 mL) were added Triethylamine (435.60 mg, 4.30 mmol, 0.6 mL) and Sodium Sulfate (4 g, 28.16 mmol, 1.49 mL). The resulting reaction mixture was stirred at 25° C. for 16 h. Upon completion, the reaction mixture was filtered, the filtrate was quenched with water (20 mL). The aqueous phase was extracted with CHCl₃ (20 mL). The combined organic phase was dried over Na₂SO₄ and concentrated under reduced pressure. To a stirred solution of resulting intermediate in MeOH (10 mL) was added Sodium Borohydride (0.5 g, 13.22 mmol, 465.55 μL). The resulting reaction mixture was stirred at 25° C. for 16 h. Upon completion, the reaction mixture was evaporated, quenched with water (20 mL). The aqueous phase was extracted with CHCl₃ (2*20 mL). The combined organic phase was dried over Na₂SO₄ and concentrated under reduced pressure. The desired product N-benzylchroman-4-amine (0.45 g, 1.88 mmol, 66.52% yield) was isolated.

LCMS (ESI): [M+H]⁺m/z: calcd 240.2; found 240.2; Rt=0.922 min.

### Step 2: The Synthesis of N′-Benzyl-N′-chroman-4-yl-oxamide

To a solution of N-benzylchroman-4-amine (0.45 g, 1.88 mmol) and Triethylamine (254.10 mg, 2.51 mmol, 0.35 mL) in CHCl₃ (10 mL) was added 2,2,2-trifluoroethyl 2-chloro-2-oxo-acetate (0.4 g, 2.10 mmol) at 25° C. The resulting reaction mixture was allowed to warm to room temperature and stirred for 16 h at 25° C. After 16 hr the reaction mixture was quenched with water (20 mL) and extracted with CHCl₃ (2*20 mL). The organic layer was dried over Na₂SO₄ and evaporated under reduced pressure. To a stirred solution of resulting intermediate in MeOH (5 mL) was added MeOH/NH3 (5 mL). The resulting reaction mixture was stirred at 25° C. for 3h. Upon completion, the reaction mixture was concentrated under reduced pressure. The desired product N′-benzyl-N′-chroman-4-yl-oxamide (0.55 g, 1.77 mmol, 94.25% yield) was isolated.

### Step 3: The Synthesis of N-(4-Amino-2-tetrahydropyran-2-yl-pyrazolo[4,3-c]pyridin-7-yl)-N′-benzyl-N′-chroman-4-yl-oxamide

A mixture of N′-benzyl-N′-chroman-4-yl-oxamide (330 mg, 1.06 mmol), 7-bromo-2-tetrahydropyran-2-yl-pyrazolo[4,3-c]pyridin-4-amine (442.36 mg, 1.49 mmol), copper (5 mg, 78.68 μmol), Copper (I) iodide (140 mg, 735.10 μmol, 24.91 μL), cesium carbonate (519.68 mg, 1.59 mmol) and rac-(1R,2R)—N1,N2-dimethylcyclohexane-1,2-diamine (140 mg, 984.25 μmol) in 1,4-dioxane (7 mL) was stirred in a sealed vial under argon at 105° C. for 48 hr. The resulting mixture was cooled down and filtered. The filtercake was washed successively with THF (2*5 mL) and dichloromethane (3*5 ml). The combined filtrate was concentrated in vacuo to afford crude N-(4-amino-2-tetrahydropyran-2-yl-pyrazolo[4,3-c]pyridin-7-yl)-N′-benzyl-N′-chroman-4-yl-oxamide (1.2 g, crude) as light-brown gum, which was used directly in the next step.

LCMS (ESI): [M+H]⁺m/z: calcd 525.2; found 525.2; Rt=2.861 min.

### Step 4: The Synthesis of N-(4-Amino-1H-pyrazolo[4,3-c]pyridin-7-yl)-N′-benzyl-N′-chroman-4-yl-oxamide (Compound 97)

Hydrogen chloride solution 4.0M in dioxane (5.25 g, 20.02 mmol, 5 mL, 13.9% purity) was added to a stirred solution of crude from previous step N-(4-amino-2-tetrahydropyran-2-yl-pyrazolo[4,3-c]pyridin-7-yl)-N′-benzyl-N′-chroman-4-yl-oxamide (1.2 g, 2.28 mmol) in methanol (5 mL) at 25° C. The resulting solution was stirred at 25° C. for 12 hr, then concentrated to dryness in vacuo and the residue was submitted to reverse phase HPLC (column: Chromatorex 18 SMB 100-5T 100×19 mm 5 μm; mobile phase: 15-45% 0-5 min H₂O/Acetonitrile/0.1% FA; flow: 30 mL/min (loading pump 4 mL/min water)) to afford Compound 97 N-(4-amino-1H-pyrazolo[4,3-c]pyridin-7-yl)-N′-benzyl-N′-chroman-4-yl-oxamide (61 mg, 124.87 μmol, 5.48% yield, HCOOH) as light-yellow solid.

¹H NMR (600 MHz, DMSO-d₆) δ (ppm) 1.61-2.04 (m, 1H), 2.04-2.26 (m, 1H), 3.78-3.90 (m, 1H), 4.02-4.41 (m, 2H), 4.55-5.09 (m, 1H), 5.19-5.81 (m, 1H), 6.26-6.81 (m, 3H), 6.85-6.96 (m, 1H), 7.01-7.10 (m, 2H), 7.11-7.17 (m, 1H), 7.17-7.26 (m, 2H), 7.27-7.37 (m, 2H), 7.48-7.74 (m, 1H), 8.14-8.23 (m, 1H), 9.68-10.92 (m, 1H), 12.27-14.08 (m, 2H).

LCMS (ESI): [M+H]⁺m/z: calcd 443.2; found 443.2; Rt=2.260 min.

### Example 122. Synthesis of rac-N-(4-Amino-1H-pyrazolo[4,3-c]pyridin-7-yl)-N′-benzyl-N′-tetralin-1-yl-oxamide (Compound 141)

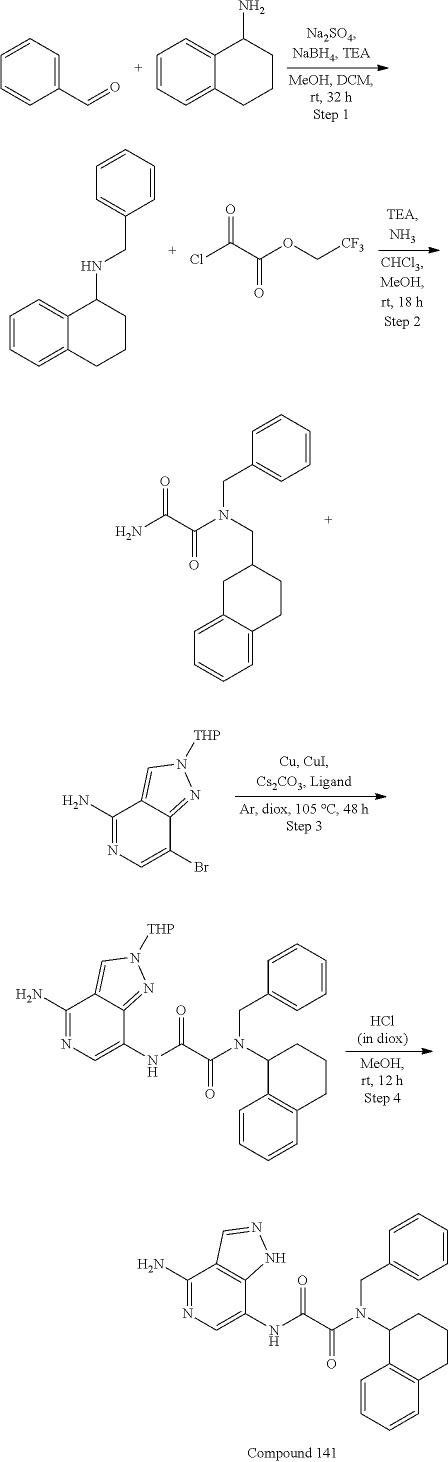

### Step 1: The Synthesis of N-Benzyltetralin-1-amine

To a stirred solution of benzaldehyde (0.3 g, 2.83 mmol) and tetralin-1-amine (0.42 g, 2.85 mmol) in DCM (10 mL), Sodium Sulfate (4 g, 28.16 mmol, 1.49 mL) was added. The resulting reaction mixture was stirred at 25° C. for 16 h. Upon completion, the reaction mixture was filtered, the filtrate was quenched with water (20 mL). The aqueous phase was extracted with CHCl₃ (20 mL). The combined organic phase was dried over Na₂SO₄ and concentrated under reduced pressure. To a stirred solution of resulting intermediate in MeOH (10 mL) was added Sodium Borohydride (0.5 g, 13.22 mmol, 465.55 μL). The resulting reaction mixture was stirred at 25° C. for 16 h. Upon completion, the reaction mixture was evaporated, quenched with water (20 mL). The aqueous phase was extracted with CHCl₃ (2*20 mL). The combined organic phase was dried over Na₂SO₄ and concentrated under reduced pressure. The desired product N-benzyltetralin-1-amine (0.45 g, 1.90 mmol, 67.07% yield) was isolated.

LCMS (ESI): [M+H]⁺m/z: calcd 238.2; found 238.2; Rt=1.011 min.

### Step 2: The Synthesis of N′-Benzyl-N′-tetralin-1-yl-oxamide

To a solution of N-benzyltetralin-1-amine (0.45 g, 1.90 mmol) and Triethylamine (254.10 mg, 2.51 mmol, 0.35 mL) in CHCl₃ (10 mL) was added 2,2,2-trifluoroethyl 2-chloro-2-oxo-acetate (0.4 g, 2.10 mmol) at 25° C. The resulting reaction mixture was allowed to warm to room temperature and stirred for 16 h at 25° C. After 16 hr the reaction mixture was quenched with water (20 mL) and extracted with CHCl₃ (2*20 mL). The organic layer was dried over Na₂SO₄ and evaporated under reduced pressure. To a stirred solution of resulting intermediate in MeOH (5 mL) was added MeOH/NH3 (5 mL). The resulting reaction mixture was stirred at 25° C. for 3h. Upon completion, the reaction mixture was concentrated under reduced pressure. The desired product N′-benzyl-N′-tetralin-1-yl-oxamide (0.55 g, 1.78 mmol, 94.07% yield) was isolated.

### Step 3: The Synthesis of N-(4-Amino-2-tetrahydropyran-2-yl-pyrazolo[4,3-c]pyridin-7-yl)-N′-benzyl-N′-tetralin-1-yl-oxamide

A mixture of N′-benzyl-N′-tetralin-1-yl-oxamide (300 mg, 972.85 μmol), 7-bromo-2-tetrahydropyran-2-yl-pyrazolo[4,3-c]pyridin-4-amine (433.62 mg, 1.46 mmol), copper (5 mg, 78.68 μmol), Copper (I) iodide (140 mg, 735.10 μmol, 24.91 μL), cesium carbonate (507.16 mg, 1.56 mmol) and rac-(1R,2R)—N1,N2-dimethylcyclohexane-1,2-diamine (140 mg, 984.25 μmol) in 1,4-dioxane (7 mL) was stirred in a sealed vial under argon at 105° C. for 48 hr. The resulting mixture was cooled down and filtered. The filtercake was washed successively with THF (2*5 mL) and dichloromethane (3*5 mL). The combined filtrate was concentrated in vacuo to afford crude N-(4-amino-2-tetrahydropyran-2-yl-pyrazolo[4,3-c]pyridin-7-yl)-N′-benzyl-N′-tetralin-1-yl-oxamide (1.1 g, crude) as brown gum, which was used directly in the next step.

LCMS (ESI): [M+H]⁺m/z: calcd 525.2; found 525.2; Rt=3.461 min.

### Step 4: The Synthesis of N-(4-Amino-1H-pyrazolo[4,3-c]pyridin-7-yl)-N′-benzyl-N′-tetralin-1-yl-oxamide (Compound 141)

Hydrogen chloride solution 4.0M in dioxane (5.25 g, 20.02 mmol, 5 mL, 13.9% purity) was added to a stirred solution of crude from previous step N-(4-amino-2-tetrahydropyran-2-yl-pyrazolo[4,3-c]pyridin-7-yl)-N′-benzyl-N′-tetralin-1-yl-oxamide (1.1 g, 2.10 mmol) in methanol (5 mL) at 25° C. The resulting solution was stirred at 25° C. for 12 hr, then concentrated to dryness in vacuo and the residue was submitted to reverse phase HPLC (column: Chromatorex 18 SMB100-5T 100×19 mm 5 μm; mobile phase: 10-35% 0-5 min H₂O/Acetonitrile/0.1% FA; flow: 30 mL/min (loading pump 4 ml/min acetonitrile)) to afford Compound 141 N-(4-amino-1H-pyrazolo[4,3-c]pyridin-7-yl)-N′-benzyl-N′-tetralin-1-yl-oxamide (20 mg, 41.11 μmol, 1.96% yield, HCOOH) as light-yellow solid.

¹H NMR (600 MHz, dmso) δ 1.52-1.74 (m, 2H), 1.76-2.19 (m, 2H), 2.59-2.76 (m, 2H), 3.53-3.82 (m, 1H), 4.26-4.76 (m, 1H), 4.94-5.60 (m, 1H), 6.30-6.80 (m, 2H), 7.03-7.08 (m, 1H), 7.10-7.16 (m, 2H), 7.17-7.22 (m, 2H), 7.22-7.25 (m, 1H), 7.26-7.29 (m, 1H), 7.30-7.35 (m, 1H), 7.44-7.73 (m, 1H), 8.06-8.23 (m, 2H), 9.57-10.81 (m, 1H), 12.15-13.69 (m, 2H).

LCMS (ESI): [M+H]⁺m/z: calcd 441.2; found 441.2; Rt=2.529 min.

### Example 123. Synthesis of N-(4-amino-2H-pyrazolo[4,3-c]pyridin-7-yl)-N′-ethyl-N′-(1-isoquinolylmethyl)oxamide (Compound 146)

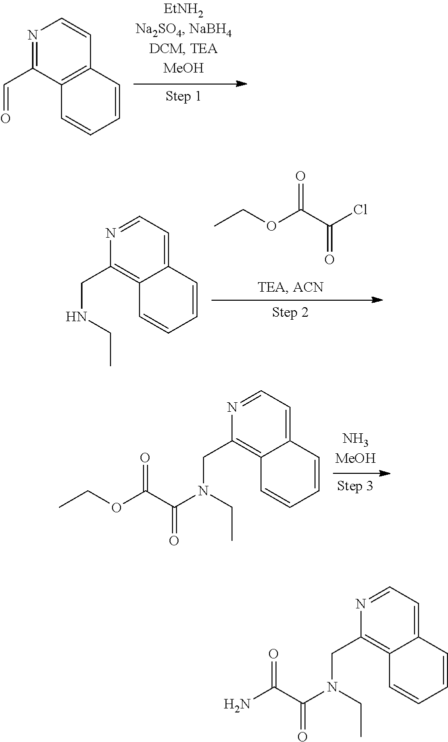

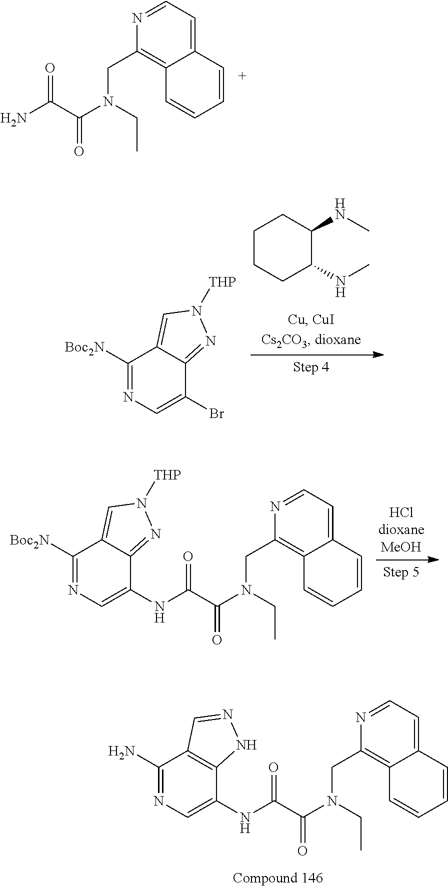

### Step 1: N-(1-isoquinolylmethyl)ethanamine

Isoquinoline-1-carbaldehyde (1 g, 6.36 mmol), Sodium sulfate, anhydrous (903.74 mg, 6.36 mmol, 337.22 μL) and ethanamine (570.72 mg, 7.00 mmol, 710.73 μL, HCl) were mixed in DCM at 20° C. The resulting mixture was stirred at 20° C. for 12 hr, then filtered and concentrated in vacuo. The residue was dissolved in methanol, cooled to 5° C. and Sodium Borohydride (264.77 mg, 7.00 mmol, 246.52 μL) was added. The reaction mixture was allowed to warm to 20° C. and stirred for 2 hr, then concentrated in vacuo. The residue was diluted with water (25 mL) and extracted with DCM (40 mL). The organic layer was separated, dried over anhydrous sodium sulfate and concentrated in vacuo to afford N-(1-isoquinolylmethyl)ethanamine (1.1 g, crude) as a red liquid.

LCMS (ESI): [M+H]⁺m/z: calcd 187.12; found 187.0; Rt=0.599 min.

### Step 2: ethyl 2-[ethyl(1-isoquinolylmethyl)amino]-2-oxo-acetate

N-(1-isoquinolylmethyl)ethanamine (1.1 g, 4.13 mmol) and TEA (836.67 mg, 8.27 mmol, 1.15 mL) were dissolved in acetonitrile (20.96 mL), cooled with ice-water bath and then ethyl 2-chloro-2-oxoacetate (733.79 mg, 5.37 mmol, 600.48 μL) was added. Solution was stirred overnight at 20° C. The RM was concentrated in vacuo, redissolved in DCM (40 mL) and washed with water twice. Organic phase was dried over anhydrous sodium sulfate and concentrated in vacuo to give ethyl 2-[ethyl(1-isoquinolylmethyl)amino]-2-oxo-acetate (1.25 g, crude) as a dark-red liquid.

LCMS (ESI): [M+H]⁺m/z: calcd 287.14; found 287.1; Rt=1.216 min.

### Step 3: N′-ethyl-N′-(1-isoquinolylmethyl)oxamide

Ethyl 2-[ethyl(1-isoquinolylmethyl)amino]-2-oxo-acetate (1.25 g, 3.06 mmol) was dissolved in MeOH (20 mL) saturated with NH3 (3.06 mmol) and stirred overnight. The RM was concentrated, redissolved in DCM, washed with water, dried over anhydrous sodium sulfate and concentrated in vacuo to give N′-ethyl-N′-(1-isoquinolylmethyl)oxamide (1.2 g, crude) as a red oil.

LCMS (ESI): [M+H]⁺m/z: calcd 258.13; found 258.2; Rt=0.538 min.

### Step 4: tert-butyl N-tert-butoxycarbonyl-N-[7-[[2-[ethyl(1-isoquinolylmethyl)amino]-2-oxo-acetyl]amino]-2-tetrahydropyran-2-yl-pyrazolo[4,3-c]pyridin-4-yl]carbamate

Copper (15.56 mg, 244.86 μmol), Copper (I) iodide (300.00 mg, 1.58 mmol, 53.38 μL), cesium carbonate (585.06 mg, 1.80 mmol) were added to a stirred solution of N′-ethyl-N′-(1-isoquinolylmethyl)oxamide (300 mg, 816.21 μmol), tert-butyl N-(7-bromo-2-tetrahydropyran-2-yl-pyrazolo[4,3-c]pyridin-4-yl)-N-tert-butoxycarbonyl-carbamate (487.16 mg, 979.45 μmol) and rac-(1R,2R)—N1,N2-dimethylcyclohexane-1,2-diamine (127.71 mg, 897.83 μmol) in 1,4-dioxane (15 mL) under Ar atmosphere and stirred at 100° C. for 12 hr in closed vial. The reaction mixture was filtered, the filtrate was combined with crude product from a separate batch and evaporated in vacuo to give tert-butyl N-tert-butoxycarbonyl-N-[7-[2-[ethyl(1-isoquinolylmethyl)amino]-2-oxo-acetyl]amino]-2-tetrahydropyran-2-yl-pyrazolo[4,3-c]pyridin-4-yl]carbamate (1 g, crude) as a brown oil.

LCMS (ESI): [M+H]⁺m/z: calcd 674.37; found 674.2; Rt=1.306 min.

### Step 5: The synthesis of N-(4-amino-2H-pyrazolo[4,3-c]pyridin-7-yl)-N′-ethyl-N′-(1-isoquinolylmethyl)oxamide

Tert-butyl N-tert-butoxycarbonyl-N-[7-[2-[ethyl(1-isoquinolylmethyl)amino]-2-oxo-acetyl]amino]-2-tetrahydropyran-2-yl-pyrazolo[4,3-c]pyridin-4-yl]carbamate (1.16 g, 602.92 μmol) was dissolved in Dioxane (6 mL) saturated with HCl (10% by weight). MeOH (6 mL) was added for better solubility. The RM was stirred for 12 hr at 20° C. then concentrated in vacuo. The residue was dissolved in 4 mL of MeOH, filtered from insoluble impurities and subjected to HPLC. HPLC data: Device (Mobile Phase, Column): SYSTEM 0.5-6.5 min 10-65% H₂O+FA/ACN flow 30 mL/min (loading pump 4 mL/min ACN), Target mass 434, Column CROMATOREX Phenil C18 100×19 mm to afford N-(4-amino-2H-pyrazolo[4,3-c]pyridin-7-yl)-N′-ethyl-N′-(1-isoquinolylmethyl)oxamide (2.3 mg, 5.28 μmol, 0.876% yield, HCOOH).as a yellow solid.

¹H NMR (500 MHz, DMSO-d₆) § 2.65 (d, 3H), 4.32-4. 56 (d, 2H), 4.62-5.68 (d, 2H), 5.62 (m, 1H), 6.24-6.37 (m, 3H), 6.41-6.50 (m, 2H), 7.02-7.05 (m, 1H), 7.07-7.09 (m, 2H), 7.28-7.31 (m, 3H), 7.65-7.68 (m, 1H), 8.16 (s, 1H), 10.58 (m, 1H), 12.76 (m, 1H).

LCMS (ESI): [M+H]⁺m/z: calcd 390.18; found 390.2; Rt=1.046 min.

### Example 124. Synthesis of N-(4-amino-1H-pyrazolo[4,3-c]pyridin-7-yl)-N′-benzyl-N′-[(2-chlorophenyl)methyl]oxamide (Compound 87)

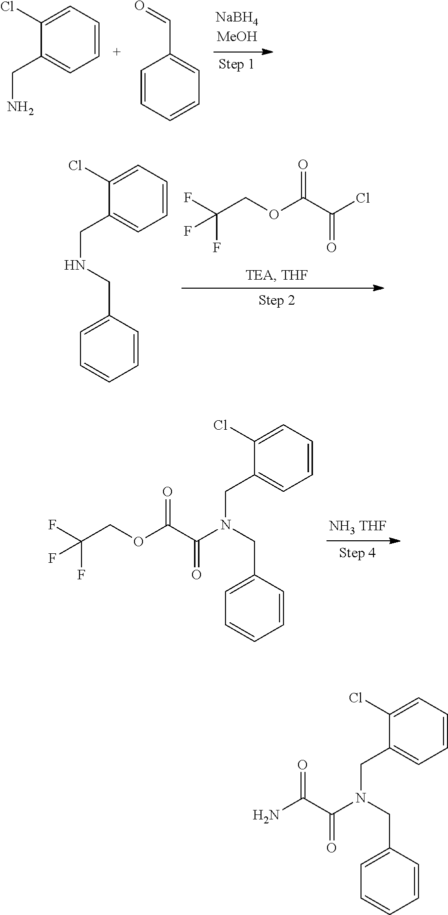

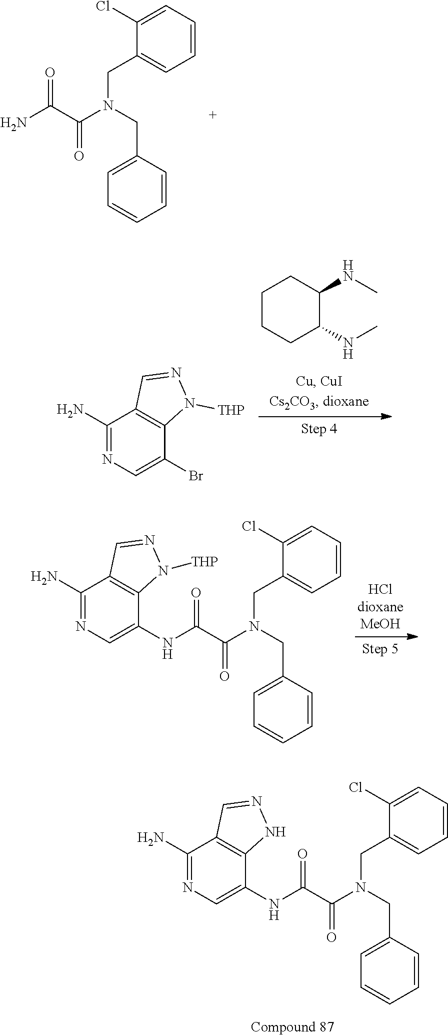

### Step 1: N-[(2-chlorophenyl)methyl]-1-phenyl-methanamine

A solution of benzaldehyde (1.2 g, 11.31 mmol) and (2-chlorophenyl)methanamine (1.60 g, 11.31 mmol, 1.37 mL) in MeOH (41.11 mL) was stirred at 20° C. for 12 hr. To this solution, Sodium Borohydride (470.55 mg, 12.44 mmol, 438.13 μL) was added and the resulting mixture was stirred for 12 hr. The solvent was removed in vacuo, the residue was taken up with water (20 mL) and extracted with DCM (3×20 mL). The combined organic layer was washed with brine (10 mL), dried over anhydrous sodium sulfate and evaporated to obtain N-[(2-chlorophenyl)methyl]-1-phenyl-methanamine (1.8 g, 7.77 mmol, 68.70% yield) as a yellow oil.

LCMS (ESI): [M+H]⁺m/z: calcd 232.09; found 232.2; Rt=2.156 min.

### Step 2:2,2,2-trifluoroethyl 2-[benzyl-[(2-chlorophenyl)methyl]amino]-2-oxo-acetate

To a solution of N-[(2-chlorophenyl)methyl]-1-phenyl-methanamine (1.8 g, 7.77 mmol) and TEA (1.18 g, 11.65 mmol, 1.62 mL) in THF (39.91 mL) was added 2,2,2-trifluoroethyl 2-chloro-2-oxo-acetate (1.78 g, 9.32 mmol) dropwise at 0° C. under argon. The reaction mixture was then stirred for 12 hr at r.t., then evaporated in vacuo to give 2,2,2-trifluoroethyl 2-[benzyl-[(2-chlorophenyl)methyl]amino]-2-oxo-acetate (2.2 g, 5.70 mmol, 73.42% yield) as a yellow oil.

LCMS (ESI): [M+H]⁺m/z: calcd 386.08; found 386.2; Rt=4.218 min.

### Step 3: N′-benzyl-N′-[(2-chlorophenyl)methyl]oxamide

2,2,2-trifluoroethyl 2-[benzyl-[(2-chlorophenyl)methyl]amino]-2-oxo-acetate (2.1 g, 5.44 mmol) was dissolved in THF (20 mL) and was blow ammonium (1.96 g, 115.33 mmol). The resulting solution was stirred at 0° C. for 14 hr. The resulting mixture was evaporated in vacuo and residue was triturated with THF (2×20 mL), filtered and combined organic layers were evaporated in vacuo to leave 1.7 g of crude product which was purification by column chromatography on silica gel using CHCl₃/MTBE gradient (10-100% MTBE) to afford N′-benzyl-N′-[(2-chlorophenyl)methyl]oxamide (1 g, 3.30 mmol, 60.68% yield) as a yellow oil.

LCMS (ESI): [M+H]⁺m/z: calcd 303.09; found 303.0; Rt=1.184 min.

### Step 4: N-(4-amino-1-tetrahydropyran-2-yl-pyrazolo[4,3-c]pyridin-7-yl)-N′-benzyl-N′-[(2-chlorophenyl)methyl]oxamide

7-bromo-1-tetrahydropyran-2-yl-pyrazolo[4,3-c]pyridin-4-amine (412.23 mg, 1.39 mmol), N′-benzyl-N′-[(2-chlorophenyl)methyl]oxamide (0.3 g, 990.90 μmol), Cu(3.15 mg, 49.55 μmol), CuI (0.15 g, 787.61 μmol, 26.69 μL), cesium carbonate (484.28 mg, 1.49 mmol) and (1R,2R)—N1,N2-dimethylcyclohexane-1,2-diamine (0.15 g, 1.05 mmol) were mixed in dioxane (6.00 mL), purged with Ar for 15 minutes and then heated in the sealed tube at 105° C. for 48 hr. Final mixture was filtered and dioxane was evaporated in vacuo. The crude product 0.55 g was purified by RP-HPLC (column: XBridge C18 5 μm 130A; 50-100% 0-5 min H₂O/CH₃OH/0.1% NH₄OH, flow: 30 mL/min) to give N-(4-amino-1-tetrahydropyran-2-yl-pyrazolo[4,3-c]pyridin-7-yl)-N′-benzyl-N′-[(2-chlorophenyl)methyl]oxamide (0.0846 g, 163.01 μmol, 16.45% yield) as a black gum.

LCMS (ESI): [M+H]⁺m/z: calcd 519.22; found 519.0; Rt=3.267 min.

### Step 5: The synthesis of N-(4-amino-1H-pyrazolo[4,3-c]pyridin-7-yl)-N′-benzyl-N′-[(2-chlorophenyl)methyl]oxamide

Hydrogen chloride, 4M in 1,4-dioxane, 99% (1.60 g, 43.88 mmol, 2 mL) was added to a solution of N-(4-amino-1-tetrahydropyran-2-yl-pyrazolo[4,3-c]pyridin-7-yl)-N′-benzyl-N′-[(2-chlorophenyl)methyl]oxamide (0.0846 g, 163.01 μmol) in MeOH (6 mL). The reaction mixture was stirred at 20° C. for 12 hr, then evaporated. The residue was purified by RP-HPLC (column: XBridge BEH18 SMB100-BT 100×19 mm; 5-55% 0-5 min H₂O/CH₃CN/0.1% FA, flow: 30 mL/min) to give N-(4-amino-1H-pyrazolo[4,3-c]pyridin-7-yl)-N′-benzyl-N′-[(2-chlorophenyl)methyl]oxamide (38.20 mg, 87.84 μmol, 53.89% yield) as a light-yellow solid.

¹H NMR (500 MHz, DMSO-d₆) δ 4.55 (s, 2H), 4.67-4.94 (m, 2H), 7.20-7.48 (m, 10H), 7.83-8.03 (m, 1H), 8.31-9.30 (m, 3H), 10.90-11.21 (m, 1H), 12.00-13.03 (m, 1H), 14.00-14.98 (m, 1H).

LCMS (ESI): [M+H]⁺m/z: calcd 435.15; found 435.2; Rt=2.828 min.

### Example 125. Synthesis of N-(4-amino-1H-pyrazolo[4,3-c]pyridin-7-yl)-N′-(cyclopropylmethyl)-N′-(o-tolylmethyl)oxamide (Compound 155)

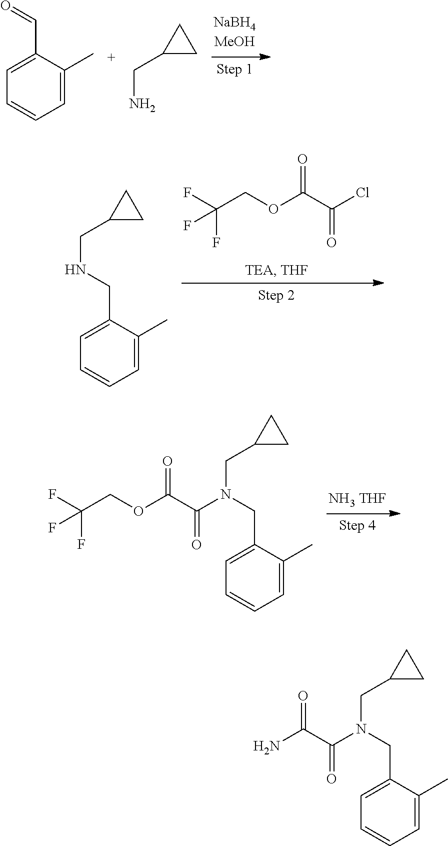

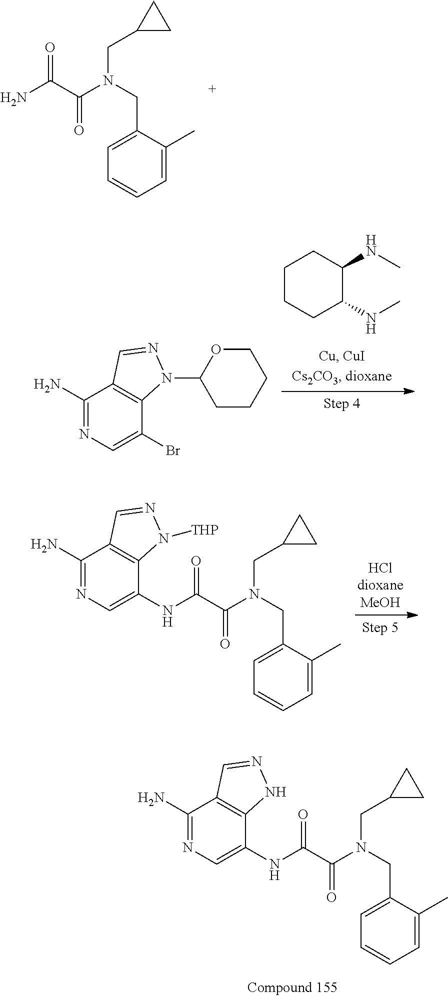

### Step 1: N-(cyclopropylmethyl)-1-(o-tolyl)methanamine

A solution of cyclopropylmethanamine (1.33 g, 18.73 mmol, 1.62 mL) and 2-methylbenzaldehyde (1.5 g, 12.48 mmol, 1.44 mL) in MeOH (40 mL) was stirred at 20° C. for 12 hr. To this solution, Sodium Borohydride (519.52 mg, 13.73 mmol, 483.73 μL) was added and the resulting mixture was stirred for 12 hr. The solvent was removed in vacuo, the residue was taken up with water (20 mL) and extracted with DCM (3×20 mL). The combined organic layer was washed with brine (10 mL), dried over anhydrous sodium sulfate and evaporated to obtain N-(cyclopropylmethyl)-1-(o-tolyl)methanamine (1.8 g, 10.27 mmol, 82.26% yield) as a yellow oil.

### Step 2:2,2,2-trifluoroethyl 2-[cyclopropylmethyl(o-tolylmethyl)amino]-2-oxo-acetate

To a solution of N-(cyclopropylmethyl)-1-(o-tolyl)methanamine (1.8 g, 10.27 mmol) and TEA (1.56 g, 15.40 mmol, 2.15 mL) in THF (50 mL) was added 2,2,2-trifluoroethyl 2-chloro-2-oxo-acetate (2.35 g, 12.32 mmol) dropwise at 0° C. under argon. The reaction mixture was then stirred for 8 hr at r.t., then evaporated in vacuo to give 2,2,2-trifluoroethyl 2-[cyclopropylmethyl(o-tolylmethyl)amino]-2-oxo-acetate (2.2 g, 6.68 mmol, 65.05% yield) as a light-yellow oil.

### Step 3: N′-(cyclopropylmethyl)-N′-(o-tolylmethyl)oxamide

2,2,2-trifluoroethyl 2-[cyclopropylmethyl(o-tolylmethyl)amino]-2-oxo-acetate (2.2 g, 6.68 mmol) was dissolved in THF (40 mL) and was blow ammonium (113.77 mg, 6.68 mmol). The resulting solution was stirred at 0° C. for 5 hr. The resulting mixture was evaporated in vacuo and residue was triturated with THF (2×20 mL), filtered and combined organic layer was evaporated in vacuo to leave 1.6 g of crude product which was purification by column chromatography on silica gel using CHCl₃/CH₃CN gradient (10-100% CH₃CN) to afford N′-(cyclopropylmethyl)-N′-(o-tolylmethyl)oxamide (1.1 g, 4.47 mmol, 66.85% yield) as a light-yellow oil.

LCMS (ESI): [M+H]⁺m/z: calcd 247.15; found 247.0; Rt=1.057 min.

### Step 4: N-(4-amino-1-tetrahydropyran-2-yl-pyrazolo[4,3-c]pyridin-7-yl)-N′-(cyclopropylmethyl)-N′-(o-tolylmethyl)oxamide

7-bromo-1-tetrahydropyran-2-yl-pyrazolo[4,3-c]pyridin-4-amine (242.97 mg, 817.68 μmol), N′-(cyclopropylmethyl)-N′-(o-tolylmethyl)oxamide (154.92 mg, 628.98 μmol), Cu(0.01 g, 157.37 μmol), CuI (0.15 g, 787.61 μmol, 26.69 μL), cesium carbonate (307.40 mg, 943.48 μmol) and (1R,2R)—N1,N2-dimethylcyclohexane-1,2-diamine (0.15 g, 1.05 mmol) were mixed in dioxane (6 mL), purged with Ar for 15 minutes and then heated in the sealed tube at 105° C. for 48 hr. Final mixture was filtered and dioxane was evaporated in vacuo. The crude product 0.45 g was purified by RP-HPLC (column: XBridge C18 5 μm 130A; 30-30-60% 0-1.5-5 min H₂O/CH₃CN/0.1% NH₄OH, flow: 30 mL/min) to give N-(4-amino-1-tetrahydropyran-2-yl-pyrazolo[4,3-c]pyridin-7-yl)-N′-(cyclopropylmethyl)-N′-(o-tolylmethyl)oxamide (0.0616 g, 133.18 μmol, 21.17% yield) as a brown solid.

LCMS (ESI): [M+H]⁺m/z: calcd 463.28; found 463.2; Rt=3.188 min.

### Step 5: The synthesis of N-(4-amino-1H-pyrazolo[4,3-c]pyridin-7-yl)-N′-(cyclopropylmethyl)-N′-(o-tolylmethyl)oxamide

Hydrogen chloride solution 4.0M in dioxane (4.00 g, 109.71 mmol, 5 mL) was added to a solution of N-(4-amino-1-tetrahydropyran-2-yl-pyrazolo[4,3-c]pyridin-7-yl)-N′-(cyclopropylmethyl)-N′-(o-tolylmethyl)oxamide (0.0616 g, 133.18 μmol) in MeOH (20 mL). The reaction mixture was stirred at 20° C. for 24 hr, then evaporated was purified by RP-HPLC (column: XBridge BEH18 SMB100-BT 100×19 mm; Oct. 10, 2040% 0-1.5-5 min H₂O/CH₃OH/0.1% NH₄OH, flow: 30 mL/min) to give N-(4-amino-1H-pyrazolo[4,3-c]pyridin-7-yl)-N′-(cyclopropylmethyl)-N′-(o-tolylmethyl)oxamide (27.90 mg, 73.73 μmol, 55.36% yield) as a light-yellow solid.

¹H NMR (500 MHz, DMSO-d₆) δ 0.07-0.24 (m, 2H), 0.32-0.49 (m, 2H), 0.74-1.18 (m, 1H), 1.99-2.30 (m, 3H), 2.94-3.37 (m, 2H), 4.28-4.96 (m, 2H), 6.50-6.92 (m, 2H), 6.94-7.25 (m, 4H), 7.45-7.87 (m, 1H), 8.06-8.25 (m, 1H), 9.54-10.56 (m, 1H), 12.42-13.51 (m, 1H).

LCMS (ESI): [M+H]⁺m/z: calcd 379.19; found 379.2; Rt=2.675 min.

### Example 126. Synthesis of N-(4-amino-2H-pyrazolo[4,3-c]pyridin-7-yl)-N′-[(3-methyl-2-pyridyl)methyl]-N′-(2-pyridylmethyl)oxamide (Compound 180)

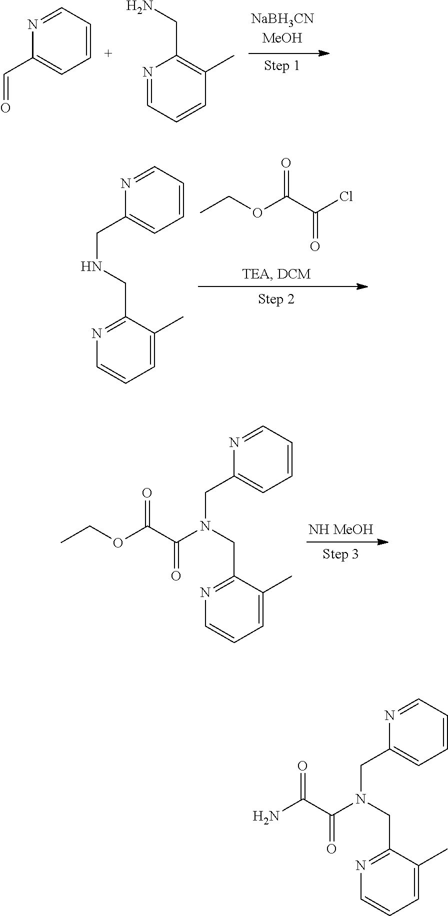

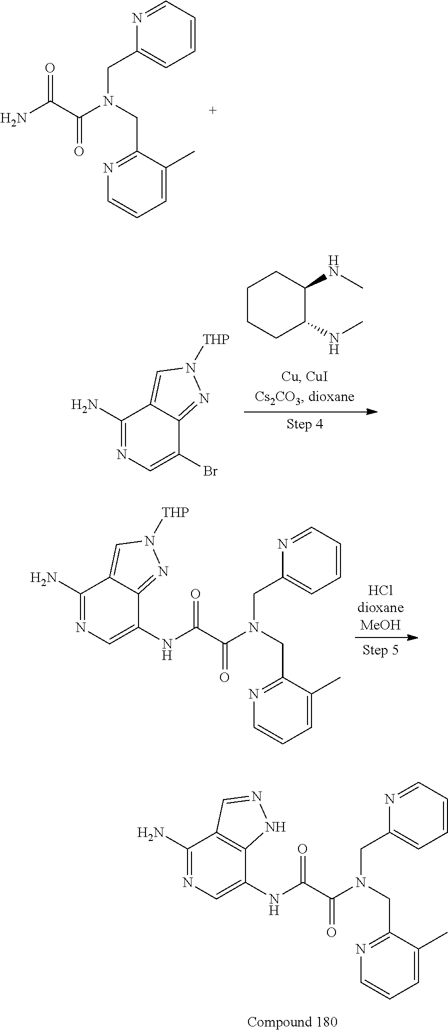

### Step 1: N-[(3-methyl-2-pyridyl)methyl]-1-(2-pyridyl)methanamine

Pyridine-2-carbaldehyde (0.6 g, 5.60 mmol, 533.81 μL) and (3-methyl-2-pyridyl)methanamine (684.35 mg, 5.60 mmol) were dissolved in Methanol (15 mL) and the resulting mixture was stirred for 30 min. Sodium cyanoborohydride (528.02 mg, 8.40 mmol) was added portionwise to the previous solution and the resulting mixture was stirred overnight. Then, solvent was removed under reduced pressure and residue was partitioned between 10% aq. K₂CO₃ solution (10 mL) and DCM (20 mL). Organic layer was separated, dried over anhydrous sodium sulfate and concentrated under reduced pressure to give N-[(3-methyl-2-pyridyl)methyl]-1-(2-pyridyl)methanamine (1.1 g, 5.16 mmol, 92.07% yield) as a red oil.

LCMS (ESI): [M+H]⁺m/z: calcd 214.14; found 214.2; Rt=0.724 min.

### Step 2: methyl 2-[(3-methyl-2-pyridyl)methyl-(2-pyridylmethyl)amino]-2-oxo-acetate

N-[(3-methyl-2-pyridyl)methyl]-1-(2-pyridyl)methanamine (250 mg, 1.17 mmol) and TEA (130.47 mg, 1.29 mmol, 179.72 μL) were dissolved in DCM (12 mL) and cooled to 0° C., following by the dropwise addition of methyl 2-chloro-2-oxo-acetate (143.60 mg, 1.17 mmol) and the reaction mixture was stirred for 14 hr at RT. The mixture was diluted with DCM, washed with water and brine. Organic layer was dried over anhydrous sodium sulfate and evaporated under reduced pressure to give methyl 2-[(3-methyl-2-pyridyl)methyl-(2-pyridylmethyl)amino]-2-oxo-acetate (310 mg, crude) as a brown oil which was used in the next step without further purification.

LCMS (ESI): [M+H]⁺m/z: calcd 300.14; found 300.2; Rt=0.591 min.

### Step 3: N′-[(3-methyl-2-pyridyl)methyl]-N′-(2-pyridylmethyl)oxamide

A solution of methyl 2-[(3-methyl-2-pyridyl)methyl-(2-pyridylmethyl)amino]-2-oxo-acetate (310 mg, 1.04 mmol) in Methanol/NH3 (5N)(5 mL) was stirred at 20° C. for 16 hr. The solvent was evaporated and the residue was purified by HPLC (2-10 min 30-50% MeOH+NH3, 30 mL/min ((loading pump 4 mL MeOH+NH3) column: XBridge BEH C18 100×20 5 microM) to give N′-[(3-methyl-2-pyridyl)methyl]-N′-(2-pyridylmethyl)oxamide (78 mg, 274.35 μmol, 26.49% yield) as a brown gum.

LCMS (ESI): [M+H]⁺m/z: calcd 285.16; found 285.2; Rt=0.350 min.

### Step 4: N-(4-amino-2-tetrahydropyran-2-yl-pyrazolo[4,3-c]pyridin-7-yl)-N′-[(3-methyl-2-pyridyl)methyl]-N′-(2-pyridylmethyl)oxamide

N′-[(3-methyl-2-pyridyl)methyl]-N′-(2-pyridylmethyl)oxamide (77 mg, 270.83 μmol), 7-bromo-2-tetrahydropyran-2-yl-pyrazolo[4,3-c]pyridin-4-amine (96.57 mg, 324.99 μmol), Copper (I) iodide (10.32 mg, 54.17 μmol, 1.84 μL), Cesium carbonate (176.48 mg, 541.66 μmol) and (1R,2R)—N1,N2-dimethylcyclohexane-1,2-diamine (46.23 mg, 324.99 μmol) were mixed in dioxane (4 mL) under argon, and then stirred overnight at 100° C. for 36 hr in vial. The reaction mixture was filtered and the filtrate was concentrated in vacuo to give N-(4-amino-2-tetrahydropyran-2-yl-pyrazolo[4,3-c]pyridin-7-yl)-N′-[(3-methyl-2-pyridyl)methyl]-N′-(2-pyridylmethyl)oxamide (210 mg, crude) as a brown gum.

LCMS (ESI): [M+H]⁺m/z: calcd 501.24; found 501.2; Rt=0.616 min.

### Step 5: The synthesis of N-(4-amino-2H-pyrazolo[4,3-c]pyridin-7-yl)-N′-[(3-methyl-2-pyridyl)methyl]-N′-(2-pyridylmethyl)oxamide

To a solution of N-(4-amino-2-tetrahydropyran-2-yl-pyrazolo[4,3-c]pyridin-7-yl)-N′-[(3-methyl-2-pyridyl)methyl]-N′-(2-pyridylmethyl)oxamide (210 mg, 419.54 μmol) in MeOH (3 mL) was added Hydrogen chloride solution 4.0M in dioxane (800.00 mg, 21.94 mmol, 1 mL) at 20° C. The resulting mixture was left to stirred for 16 hr. The resulting mixture was evaporated to dryness and then submitted to reverse phase HPLC (SYSTEM 15-40% 0-2-10 min H₂O/ACN/0.1NH4OH, flow 30 mL/min ((loading pump 4 mL ACN) target mass 416 column: Chromatorex C18 SMB100-5T 100×19 mm, 5 microM) to afford N-(4-amino-2H-pyrazolo[4,3-c]pyridin-7-yl)-N′-[(3-methyl-2-pyridyl)methyl]-N′-(2-pyridylmethyl)oxamide (14.2 mg, 34.10 μmol, 8.13% yield) as an orange solid.

¹H NMR (500 MHz, DMSO-d₆) δ 4.60-4.79 (m, 2H), 4.83-5.00 (m, 2H), 6.70 (s, 1H), 7.28-7.44 (m, 11H), 8.13 (s, 1H), 8.22-8.27 (m, 1H), 8.81-8.87 (m, 1H), 12.35 (s, 1H).

LCMS (ESI): [M+H]⁺m/z: calcd 417.19; found 417.2; Rt=0.327 min.

### Example 127. Synthesis of (R)—N¹-(4-amino-1H-pyrazolo[4,3-c]pyridin-7-yl)-N2-(2-methylbenzyl)-N2-(3-methylbutan-2-yl)oxalamide (Compound 177)

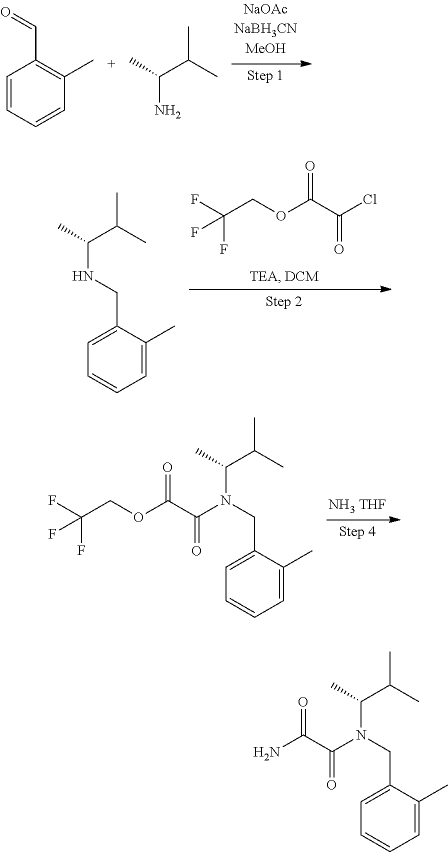

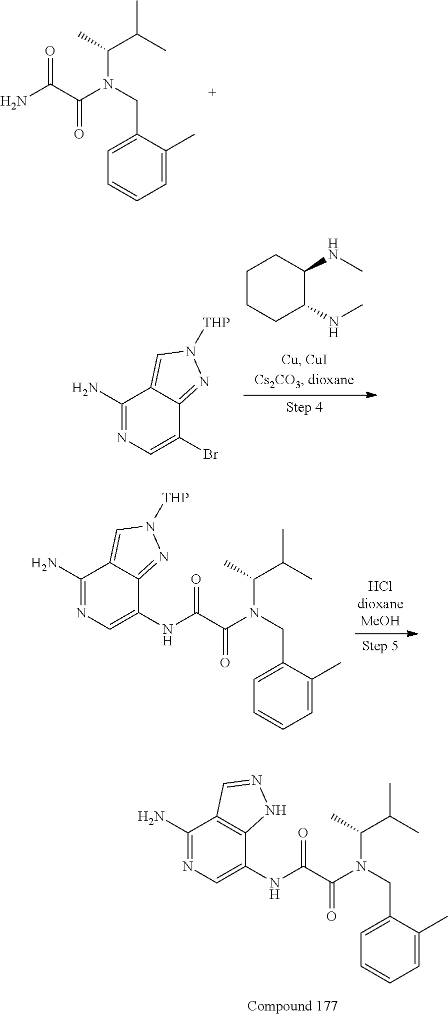

### Step 1: (R)-3-methyl-N-(2-methylbenzyl) butan-2-amine

(R)-3-methylbutan-2-amine (514.46 mg, 4.16 mmol, 689.63 μL, HCl) was dissolved in MeOH (5.5 mL) and Sodium acetate, anhydrous (375.51 mg, 4.58 mmol, 245.75 μL) was added. The resulting mixture was stirred for 15 min and 2-methylbenzaldehyde (0.5 g, 4.16 mmol, 484.12 μL) was added. The resulting mixture was stirred for 1.5 hr and Sodium cyanoborohydride (313.81 mg, 4.99 mmol) was added. The resulting mixture was stirred overnight. The reaction mixture was concentrated in vacuo and aq. NaHCO₃ solution (25 mL) was added. The resulting mixture was extracted with MTBE (2×30 mL). Combined organic layers were dried over anhydrous sodium sulfate, filtered, and concentrated in vacuo to obtain (R)-3-methyl-N-(2-methylbenzyl) butan-2-amine (739 mg, 3.86 mmol, 92.82% yield) as a light-yellow oil.

### Step 2: (R)-2,2,2-trifluoroethyl 2-((2-methylbenzyl)(3-methylbutan-2-yl)amino)-2-oxoacetate

(R)-3-methyl-N-(2-methylbenzyl) butan-2-amine (739 mg, 3.86 mmol) and Triethylamine (429.97 mg, 4.25 mmol, 592.24 μL) were dissolved in DCM (18 mL) and the resulting solution was cooled to −5° C. in an ice/methanol bath. A solution of 2,2,2-trifluoroethyl 2-chloro-2-oxo-acetate (772.68 mg, 4.06 mmol) in DCM (2 mL) was added dropwise to the previous solution. After the addition was completed, the resulting mixture was allowed to warm to room temperature and stirred overnight. Water (25 mL) was added to the reaction mixture and an organic layer was separated. The aqueous layer was extracted with DCM (20 mL) and combined organic layers were dried over anhydrous sodium sulfate, filtered, and concentrated in vacuo to obtain (R)-2,2,2-trifluoroethyl 2-((2-methylbenzyl)(3-methylbutan-2-yl)amino)-2-oxoacetate (1.33 g, crude) as a red oil.

LCMS (ESI): [M+H]⁺m/z: calcd 346.17; found 346.0; Rt=1.528 min.

### Step 3: (R)—N¹-(2-methylbenzyl)-N1-(3-methylbutan-2-yl)oxalamide

(R)-2,2,2-trifluoroethyl 2-((2-methylbenzyl)(3-methylbutan-2-yl)amino)-2-oxoacetate (1.33 g, 4.01 mmol) was dissolved in MeOH (10 mL) and NH3/MeOH (20 mL) was added thereto. The resulting mixture was stirred overnight. The reaction mixture was filtered and the filtrate was concentrated in vacuo to obtain (R)—N¹-(2-methylbenzyl)-N1-(3-methylbutan-2-yl)oxalamide (1.05 g, crude) as a yellow solid.

LCMS (ESI): [M+H]⁺m/z: calcd 263.18; found 263.2; Rt=1.085 min.

### Step 4: N1-(4-amino-2-(tetrahydro-2H-pyran-2-yl)-2H-pyrazolo[4,3-c]pyridin-7-yl)-N2-(2-methylbenzyl)-N2-((R)-3-methylbutan-2-yl)oxalamide

(R)—N¹-(2-methylbenzyl)-N1-(3-methylbutan-2-yl)oxalamide (150 mg, 571.76 μmol), 7-bromo-2-tetrahydropyran-2-yl-pyrazolo[4,3-c]pyridin-4-amine (186.89 mg, 628.94 μmol), Copper (1.82 mg, 28.59 μmol), Copper (I) iodide (54.45 mg, 285.88 μmol, 9.69 μL), rac-(1R,2R)—N1,N2-dimethylcyclohexane-1,2-diamine (61.00 mg, 428.82 μmol) and Cesium carbonate (372.58 mg, 1.14 mmol) were mixed in Dioxane (4 mL). The resulting mixture was splurged with argon for 5 min. The vial was sealed and heated at 100° C. for 45 hr. The reaction mixture was cooled and filtered. The filtercake was rinsed with MeOH (10 mL) and the filtrate was concentrated in vacuo to obtain N1-(4-amino-2-(tetrahydro-2H-pyran-2-yl)-2H-pyrazolo[4,3-c]pyridin-7-yl)-N2-(2-methylbenzyl)-N2-((R)-3-methylbutan-2-yl)oxalamide (680 mg, crude) as a greenish foam which was used in the next step without purification.

LCMS (ESI): [M+H]⁺m/z: calcd 479.28; found 479.2; Rt=0.955 min.

### Step 5: The synthesis of (R)—N¹-(4-amino-1H-pyrazolo[4,3-c]pyridin-7-yl)-N2-(2-methylbenzyl)-N2-(3-methylbutan-2-yl)oxalamide

N1-(4-amino-2-(tetrahydro-2H-pyran-2-yl)-2H-pyrazolo[4,3-c]pyridin-7-yl)-N2-(2-methylbenzyl)-N2-((R)-3-methylbutan-2-yl)oxalamide (680 mg, 1.42 mmol) was dissolved in MeOH (5 mL) and HCl/Dioxane (5 mL) was added thereto. The resulting mixture was stirred for 3 hr and then concentrated in vacuo. The residue was purified by HPLC (0-2-10 min, 30-55% H₂O/MeCN/0.1NH4OH, flow 30 mL/min ((loading pump 4 mL ACN), target mass 395, column: Chromatorex C18 SMB100-5T 100×19 mm, 5 microM) to obtain (R)—N¹-(4-amino-1H-pyrazolo[4,3-c]pyridin-7-yl)-N2-(2-methylbenzyl)-N2-(3-methylbutan-2-yl)oxalamide (71.3 mg, 180.75 μmol, 12.72% yield) as a light-yellow solid.

¹H NMR (500 MHz, DMSO-d₆) δ 0.43-0.91 (m, 6H), 0.92-1.25 (m, 3H), 1.69-2.09 (m, 1H), 2.12-2.35 (m, 3H), 3.57-3.94 (m, 1H), 4.01-4.91 (m, 2H), 6.53-6.84 (m, 2H), 6.90-7.08 (m, 1H), 7.08-7.23 (m, 3H), 7.33-7.77 (m, 1H), 7.78-8.24 (m, 1H), 9.42-10.61 (m, 1H), 11.99-13.64 (m, 1H).

LCMS (ESI): [M+H]⁺m/z: calcd 395.25; found 395.2; Rt=0.837 min.

### Example 128. The synthesis of(S)-N1-(4-amino-1H-pyrazolo[4,3-c]pyridin-7-yl)-N2-(2-methylbenzyl)-N2-(3-methylbutan-2-yl)oxalamide (Compound 117)

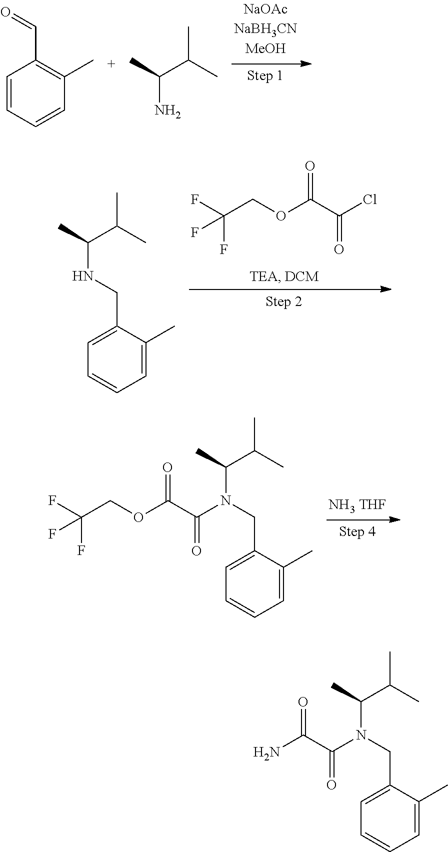

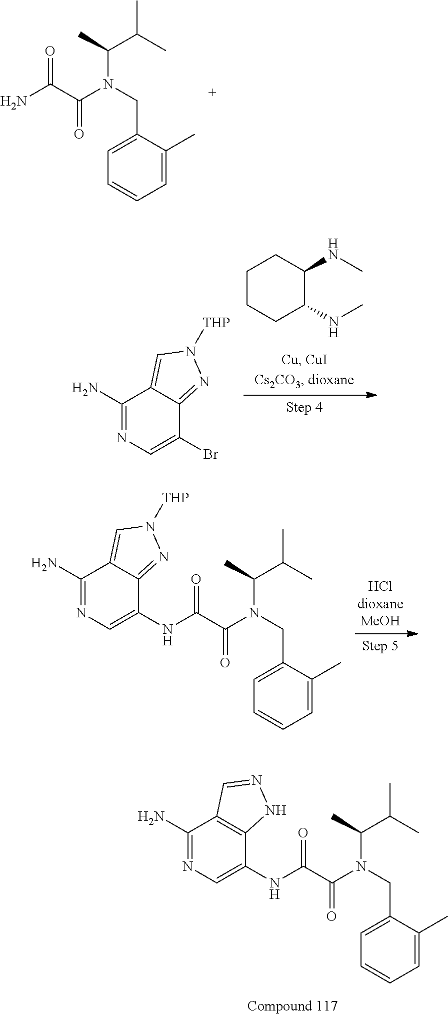

### Step 1: (S)-3-methyl-N-(2-methylbenzyl) butan-2-amine

(S)-3-methylbutan-2-amine (514.46 mg, 4.16 mmol, HCl) was dissolved in MeOH (5.5 mL) and Sodium acetate, anhydrous (375.51 mg, 4.58 mmol, 245.75 μL) was added. The resulting mixture was stirred for 15 min and 2-methylbenzaldehyde (500 mg, 4.16 mmol, 484.12 μL) was added. The resulting mixture was stirred for 1.5 hr and Sodium cyanoborohydride (313.81 mg, 4.99 mmol) was added thereto. The resulting mixture was stirred overnight. The reaction mixture was concentrated in vacuo and aq. NaHCO₃ solution (25 mL) was added. The resulting mixture was extracted with MTBE (2×30 mL). Combined organic layers were dried over anhydrous sodium sulfate, filtered, and concentrated in vacuo to obtain(S)-3-methyl-N-(2-methylbenzyl) butan-2-amine (734 mg, 3.84 mmol, 92.19% yield) as a light-yellow oil.

### Step 2: (S)-2,2,2-trifluoroethyl 2-((2-methylbenzyl)(3-methylbutan-2-yl)amino)-2-oxoacetate

(S)-3-methyl-N-(2-methylbenzyl) butan-2-amine (880 mg, 4.60 mmol) and Triethylamine (512.00 mg, 5.06 mmol, 705.24 μL) were dissolved in DCM (18 mL) and the resulting solution was cooled to -5° C. in an ice/methanol bath. A solution of 2,2,2-trifluoroethyl 2-chloro-2-oxo-acetate (920.10 mg, 4.83 mmol) in DCM (2 mL) was added dropwise to the previous solution. After the addition was completed, the resulting mixture was allowed to warm to room temperature and stirred overnight. Water (25 mL) was added to the reaction mixture and an organic layer was separated. The aqueous layer was extracted with DCM (20 mL) and combined organic layers were dried over anhydrous sodium sulfate, filtered, and concentrated in vacuo to obtain(S)-2,2,2-trifluoroethyl 2-((2-methylbenzyl)(3-methylbutan-2-yl)amino)-2-oxoacetate (1.59 g, 4.60 mmol, 100.00% yield) as a red oil.

LCMS (ESI): [M+H]⁺m/z: calcd 346.17; found 346.2; Rt=1.528 min.

### Step 3: (S)-N1-(2-methylbenzyl)-N1-(3-methylbutan-2-yl)oxalamide

(S)-2,2,2-trifluoroethyl 2-((2-methylbenzyl)(3-methylbutan-2-yl)amino)-2-oxoacetate (1.59 g, 4.60 mmol) was dissolved in MeOH (10 mL) and NH3/MeOH (20 mL) was added thereto. The resulting solution was stirred overnight. The reaction mixture was filtered and the filtrate was concentrated in vacuo to obtain(S)-N1-(2-methylbenzyl)-N1-(3-methylbutan-2-yl)oxalamide (1.20 g, 4.56 mmol, 99.02% yield) as a yellow solid.

LCMS (ESI): [M+H]⁺m/z: calcd 263.18; found 263.2; Rt=1.084 min.

### Step 4: N1-(4-amino-2-(tetrahydro-2H-pyran-2-yl)-2H-pyrazolo[4,3-c]pyridin-7-yl)-N2-(2-methylbenzyl)-N2-((S)-3-methylbutan-2-yl)oxalamide

(S)-N1-(2-methylbenzyl)-N1-(3-methylbutan-2-yl)oxalamide (150 mg, 571.76 μmol), 7-bromo-2-tetrahydropyran-2-yl-pyrazolo[4,3-c]pyridin-4-amine (186.89 mg, 628.94 μmol), Copper (1.82 mg, 28.59 μmol), Copper (I) iodide (54.45 mg, 285.88 μmol, 9.69 μL), rac-(1R,2R)—N1,N2-dimethylcyclohexane-1,2-diamine (61.00 mg, 428.82 μmol) and Cesium carbonate (372.58 mg, 1.14 mmol) were mixed in Dioxane (4 mL). The resulting mixture was splurged with argon for 5 min. The vial was sealed and heated at 100° C. for 45 hr. The reaction mixture was cooled and filtered. The filtercake was rinsed with MeOH (10 mL) and the filtrate was concentrated in vacuo to obtain N1-(4-amino-2-(tetrahydro-2H-pyran-2-yl)-2H-pyrazolo[4,3-c]pyridin-7-yl)-N2-(2-methylbenzyl)-N2-((S)-3-methylbutan-2-yl)oxalamide (708 mg, crude) as a greenish foam which was used in the next step without purification.

LCMS (ESI): [M+H]⁺m/z: calcd 479.28; found 479.2; Rt=0.954 min.

### Step 5: The synthesis of(S)-N1-(4-amino-1H-pyrazolo[4,3-c]pyridin-7-yl)-N2-(2-methylbenzyl)-N2-(3-methylbutan-2-yl)oxalamide

N1-(4-amino-2-(tetrahydro-2H-pyran-2-yl)-2H-pyrazolo[4,3-c]pyridin-7-yl)-N2-(2-methylbenzyl)-N2-((S)-3-methylbutan-2-yl)oxalamide (708 mg, 1.48 mmol) was dissolved in MeOH (5 mL) and HCl/Dioxane (5 mL) was added thereto. The resulting mixture was stirred for 3 hr and then concentrated in vacuo. The residue was purified by HPLC (0-2-10 min, 30-55% H₂O/MeCN/0.1NH4OH, flow 30 mL/min ((loading pump 4 mL MeCN), target mass 395, column: Chromatorex C18 SMB100-5T 100×19 mm, 5 microM) to obtain(S)-N1-(4-amino-1H-pyrazolo[4,3-c]pyridin-7-yl)-N2-(2-methylbenzyl)-N2-(3-methylbutan-2-yl)oxalamide (59.5 mg, 150.84 μmol, 10.20% yield) as a light-yellow solid.

¹H NMR (500 MHz, DMSO-d₆) δ 0.45-0.90 (m, 6H), 0.99-1.16 (m, 3H), 1.86-2.10 (m, 1H), 2.21-2.34 (m, 3H), 3.78-3.92 (m, 1H), 4.13-4.59 (m, 1H), 4.59-4.90 (m, 1H), 6.51-6.72 (m, 2H), 6.75-7.00 (m, 1H), 7.09-7.19 (m, 3H), 7.37-7.81 (m, 1H), 8.09-8.20 (m, 1H), 9.21-10.84 (m, 1H), 12.49-13.64 (m, 1H).

LCMS (ESI): [M+H]⁺m/z: calcd 395.25; found 395.2; Rt=1.115 min.

### Example 129. Synthesis of N-(4-Amino-1H-pyrazolo[4,3-c]pyridin-7-yl)-N′—[[2,6-difluoro-4-(trifluoromethyl)phenyl]methyl]-N′-methyl-oxamide (Compound 195)

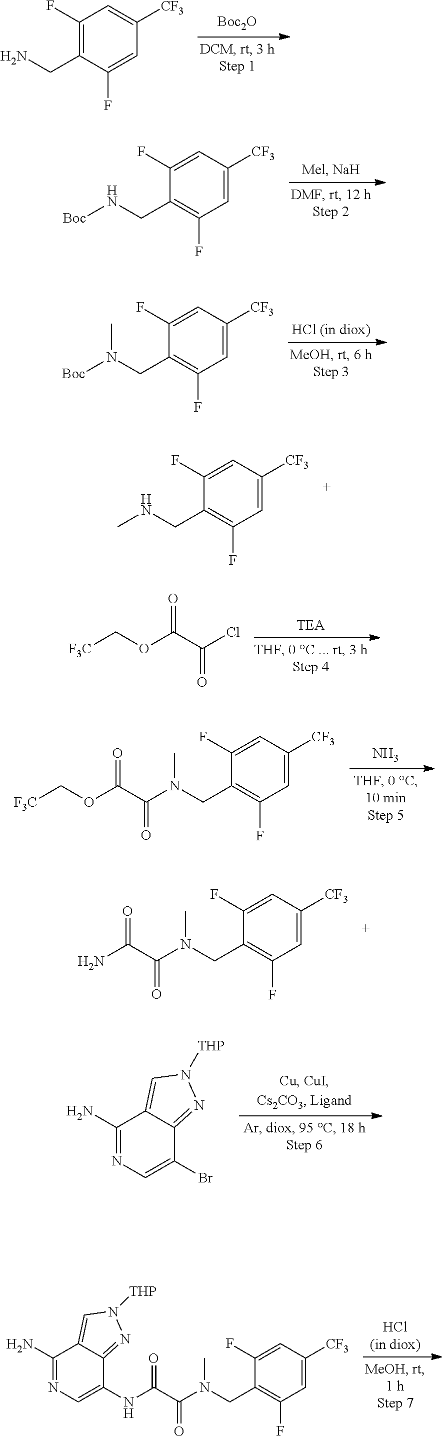

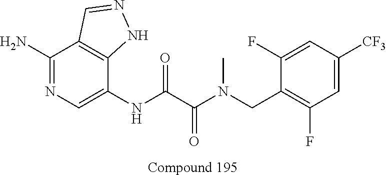 [SMILES: CN(Cc1c(F)cc(C(F)(F)F)cc1F)C(=O)C(=O)Nc1cnc(N)c2cn[nH]c12 ; "Compound 195"]

### Step 1: The Synthesis of tert-Butyl N-[[2,6-difluoro-4-(trifluoromethyl)phenyl methyl]carbamate

To a solution of [2,6-difluoro-4-(trifluoromethyl)phenyl]methanamine (2.2 g, 10.42 mmol) in DCM (25.11 mL), Di-tert-butyl dicarbonate (2.27 g, 10.42 mmol, 2.39 mL) was added. The resulting mixture was stirred at 25° C. for 3 hr and evaporated in vacuo to give tert-butyl N-[[2,6-difluoro-4-(trifluoromethyl)phenyl]methyl]carbamate (3.2 g, 10.28 mmol, 98.67% yield).

LCMS (ESI): [M-tBu]⁺m/z: calcd 256.2; found 256.0; Rt=1.466 min.

### Step 2: The Synthesis of tert-Butyl N-[[2,6-difluoro-4-(trifluoromethyl)phenyl methyl]-N-methyl-carbamate

To a stirred at 0° C. solution of tert-butyl N-[[2,6-difluoro-4-(trifluoromethyl)phenyl]methyl]carbamate (3.2 g, 10.28 mmol) in DMF (35 mL), Sodium Hydride (in oil dispersion) 60% dispersion in mineral oil (616.81 mg, 15.42 mmol, 60% purity) was added. After 30 min, Methyl iodide (2.92 g, 20.56 mmol, 1.28 mL) was added. The resulting mixture was stirred at 25° C. for 12 hr and poured into water (100 mL) and extracted with MTBE (3*15 mL), dried over Na₂SO₄ and evaporated in vacuo to obtain tert-butyl N-[[2,6-difluoro-4-(trifluoromethyl)phenyl]methyl]-N-methyl-carbamate (2 g, 6.15 mmol, 59.80% yield).

LCMS (ESI): [M-tBu]⁺m/z: calcd 270.2; found 270.0; Rt=1.465 min.

### Step 3: The Synthesis of 1-[2,6-Difluoro-4-(trifluoromethyl)phenyl]-N-methyl-methanamine

A solution of tert-butyl N-[[2,6-difluoro-4-(trifluoromethyl)phenyl]methyl]-N-methyl-carbamate (2 g, 6.15 mmol) in MeOH (20 mL) and Hydrogen chloride solution 4.0M in dioxane (10 g, 274.27 mmol) was stirred at 25° C. for 6 hr. The solvent was evaporated to give crude product, which was triturated with MTBE-hexane. The formed precipitate was filtered and dried to give 1-[2,6-difluoro-4-(trifluoromethyl)phenyl]-N-methyl-methanamine (1.5 g, 5.73 mmol, 93.25% yield, HCl).

LCMS (ESI): [M+H]⁺m/z: calcd 226.0; found 226.0; Rt=0.773 min.

### Step 4: The Synthesis of 2,2,2-Trifluoroethyl 2-[[2,6-difluoro-4-(trifluoromethyl)phenyl methyl-methyl-amino]-2-oxo-acetate

To a solution of 1-[2,6-difluoro-4-(trifluoromethyl)phenyl]-N-methyl-methanamine (1.5 g, 5.73 mmol, HCl) and Triethylamine (2.90 g, 28.67 mmol, 4.00 mL) in THF (50 mL), 2,2,2-trifluoroethyl 2-chloro-2-oxo-acetate (2.18 g, 11.47 mmol) was added portionwise at 0° C. The resulting mixture was stirred for 3 hr. LCMS showed full conversion of SM. The reaction mixture was directly used for the next step.

LCMS (ESI): [M+H]⁺m/z: calcd.380.2; found 380.0; Rt=1.310 min.

### Step 5: The Synthesis of N′—[[2,6-Difluoro-4-(trifluoromethyl)phenyl]methyl]-N′-methyl-oxamide

Through a solution of 2,2,2-trifluoroethyl 2-[[2,6-difluoro-4-(trifluoromethyl)phenyl]methyl-methyl-amino]-2-oxo-acetate (2 g, 5.27 mmol) in THF (50 mL), ammonia was bubbled during 10 min at 0° C. The formed precipitate was filtered off, washed with THF (30 mL) and the solvent was evaporated in vacuo to give crude product (1.5 g), which was purified by gradient chromatography (CHCl₃-ACN) to afford N′—[[2,6-difluoro-4-(trifluoromethyl)phenyl]methyl]-N′-methyl-oxamide (1.3 g, 4.39 mmol, 83.22% yield).

LCMS (ESI): [M+H]⁺m/z: calcd 297.2; found 297.0; Rt=1.130 min.

### Step 6: The Synthesis of N-(4-Amino-2-tetrahydropyran-2-yl-pyrazolo[4,3-c]pyridin-7-yl)-N′-[2,6-difluoro-4-(trifluoromethyl)phenyl]methyl]-N′-methyl-oxamide

To a mixture of N′-[2,6-difluoro-4-(trifluoromethyl)phenyl]methyl]-N′-methyl-oxamide (0.2 g, 675.24 μmol), 7-bromo-2-tetrahydropyran-2-yl-pyrazolo[4,3-c]pyridin-4-amine (250.81 mg, 844.04 μmol), Copper (8.58 mg, 135.05 μmol), Copper (I) iodide (77.16 mg, 405.14 μmol, 13.73 μL), Cesium carbonate (264.01 mg, 810.28 μmol) and rac-(1R,2R)—N1,N2-dimethylcyclohexane-1,2-diamine (57.63 mg, 405.14 μmol), Dioxane (5 mL) was added. The resulting mixture was evacuated, refiled with Argon three time, heated at 95° C. for 18 hr and cooled. The inorganic precipitate was filtered off and washed with DCM (50 mL) to give N-(4-amino-2-tetrahydropyran-2-yl-pyrazolo[4,3-c]pyridin-7-yl)-N′—[[2,6-difluoro-4-(trifluoromethyl)phenyl]methyl]-N′-methyl-oxamide (0.4 g, crude).

This substance was used for the next step without further purification.

LCMS (ESI): [M+H]⁺m/z: calcd 513.2; found 513.2; Rt=1.072 min.

### Step 7: The Synthesis of N-(4-Amino-1H-pyrazolo[4,3-c]pyridin-7-yl)-N′—[[2,6-difluoro-4-(trifluoromethyl)phenyl]methyl]-N′-methyl-oxamide (Compound 195)

To a solution of N-(4-amino-2-tetrahydropyran-2-yl-pyrazolo[4,3-c]pyridin-7-yl)-N′—[[2,6-difluoro-4-(trifluoromethyl)phenyl]methyl]-N′-methyl-oxamide (0.4 g, 780.59 μmol) in MeOH (20 mL), Hydrogen chloride solution 4.0M in dioxane (10 g, 274.27 mmol) was added. The resulting mixture was stirred at 25° C. for 1 hr and evaporated in vacuo. The residue was purified by HPLC (Device (Mobile Phase, Column): SYSTEM May 5, 2025% 0-1.3-6.3 min H₂O/MeCN/0.1% FA, flow: 30 mL/min (loading pump 4 mL/min MeCN); column: Chromatorex 18 SMB100-ST 100×19 mm 5 μm) to obtain N-(4-amino-1H-pyrazolo[4,3-c]pyridin-7-yl)-N′—[[2,6-difluoro-4-(trifluoromethyl)phenyl]methyl]-N′-methyl-oxamide (159 mg, 335.20 μmol, 42.94% yield, HCO₂H).

¹H NMR (600 MHz, dmso) δ 2.86-3.19 (m, 3H), 4.24-5.22 (m, 2H), 6.70 (s, 2H), 7.23-7.80 (m, 3H), 8.14-8.22 (m, 1H), 9.23-10.61 (m, 1H), 12.11-13.33 (m, 1H).

LCMS (ESI): [M+H]⁺m/z: calcd 429.2; found 429.2; Rt=2.157 min.

### Example 130. Synthesis of N-(4-Amino-1H-pyrazolo[4,3-c]pyridin-7-yl)-N′-ethyl-N′-[[2-methyl-4-(pentafluoro-sulfanyl)phenyl]methyl]oxamide (Compound 194)

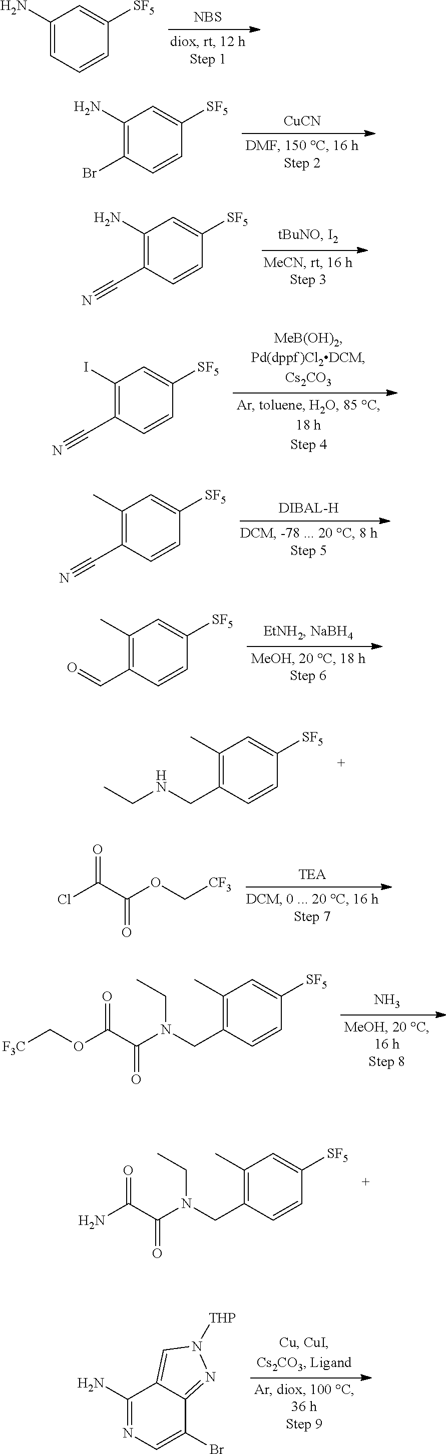

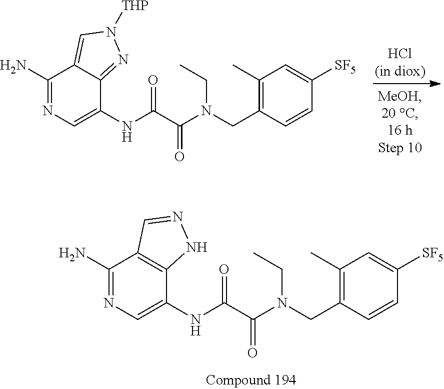

### Step 1: The Synthesis of 2-Bromo-5-(pentafluoro-sulfanyl) aniline

NBS (3.88 g, 21.80 mmol) was added portion-wise to a solution of 3-(pentafluoro-sulfanyl) aniline (4.55 g, 20.76 mmol) in dioxane (45 mL), and the reaction mixture was stirred at rt for 12 hr. Then, solvent was removed under reduced pressure and residue was partitioned between water and MTBE. Organic layer was separated, dried over Na₂SO₄ and concentrated under reduced pressure, leaving 2-bromo-5-(pentafluoro-sulfanyl) aniline (6.2 g, crude). ¹H NMR (400 MHz, CDCl₃) δ (ppm) 3.72 (br s, 2H), 6.98 (d, 1H), 7.14 (s, 1H), 7.49 (d, 1H).

### Step 2: The Synthesis of 2-Amino-4-(pentafluoro-sulfanyl)benzonitrile

A mixture of 2-bromo-5-(pentafluoro-sulfanyl) aniline (4 g, 13.42 mmol) and Copper (I) cyanide (6.01 g, 67.10 mmol, 2.06 mL) in DMF (40 mL) was heated at 150° C. for 16 hr. Then RM it was poured in water and extracted with MTBE. Combined organic extracts were washed with brine (3×10 mL), dried over anhydrous sodium sulfate, filtered and concentrated under reduced pressure to afford 2-amino-4-(pentafluoro-sulfanyl)benzonitrile (2.9 g, 11.88 mmol, 88.50% yield).

¹H NMR (500 MHz, CDCl₃) δ (ppm) 4.70 (m, 2H), 7.09 (d, 1H), 7.14 (s, 1H), 7.46 (d, 1H).

### Step 3: The Synthesis of 2-Iodo-4-(pentafluoro-sulfanyl)benzonitrile

To a solution of 2-amino-4-(pentafluoro-sulfanyl)benzonitrile (2.22 g, 9.09 mmol) in acetonitrile (50 mL) was added iodine (2.31 g, 9.09 mmol), stirred for 5 min, and then added dropwise tert-Butyl nitrite, tech. 90% (1.88 g, 18.18 mmol, 2.16 mL), stirred at room temperature for 16 hr. After the reaction was completed, the reaction solution was poured into saturated sodium thiosulfate solution, extracted with EA, the organic phase was washed with saturated brine, dried over anhydrous sodium sulfate, and concentrated in vacuo.

¹H NMR (400 MHz, CDCl₃) δ (ppm) 7.58 (d, 1H), 7.85 (d, 1H), 8.26 (s, 1H).

### Step 4: The Synthesis of 2-Methyl-4-(pentafluoro-sulfanyl)benzonitrile

2-Iodo-4-(pentafluoro-sulfanyl)benzonitrile (2.61 g, 7.35 mmol) and methylboronic acid (1.76 g, 29.40 mmol) were dissolved in toluene (50 mL) and a solution of Pd(dppf)Cl₂*DCM (537.86 mg, 735.07 μmol) in water (5 mL) was added thereto. The resulting mixture was evacuated and backfilled three times with argon. Cesium carbonate (7.19 g, 22.05 mmol) was added to the previous mixture and the resulting mixture was heated at 85° C. overnight. The reaction mixture was concentrated in vacuo and water (100 mL) was added to the residue. The resulting mixture was extracted with EtOAc (2*45 mL) and combined organic layers were washed with brine (25 mL), dried over Na₂SO₄, filtered, concentrated in vacuo and submitted to FCC to obtain 2-methyl-4-(pentafluoro-sulfanyl)benzonitrile (0.96 g, 3.95 mmol, 53.70% yield). Interchim 80 g SiO₂, CHC₁₃—CH₃CN from 0~100%, flow rate=70 mL/min, cv=1.1

¹H NMR (400 MHz, CDCl₃) δ (ppm) 2.62 (s, 3H), 7.68 (m, 3H).

### Step 5: The Synthesis of 2-Methyl-4-(pentafluoro-sulfanyl)benzaldehyde

To a solution of 2-methyl-4-(pentafluoro-sulfanyl)benzonitrile (410 mg, 1.69 mmol) in DCM (20 mL) was added DIBAL-H (287.72 mg, 2.02 mmol) dropwise at −78° C. and the reaction mixture was stirred for 8 hr at 20° C. The reaction mixture was poured into 10% H₂SO₄ (10 mL) and stirred for 1 h. The organic layer dried over sodium sulfate and concentrated under reduced pressure to obtain 2-methyl-4-(pentafluoro-sulfanyl)benzaldehyde (0.4 g, 1.62 mmol, 96.37% yield) that was used in the next step without further purification. ¹H NMR (400 MHz, CDCl₃) δ (ppm) 2.62 (s, 3H), 7.77 (m, 2H), 7.91 (m, 1H), 10.34 (s, 1H).1

### Step 6: The Synthesis of N-[[2-Methyl-4-(pentafluoro-sulfanyl)phenyl]methyl]ethanamine

2-Methyl-4-(pentafluoro-sulfanyl)benzaldehyde (242 mg, 982.95 μmol) was added to a solution of ethanamine (316.53 mg, 4.91 mmol, 394.19 μL) in methanol (10 mL) at 20° C. The resulting mixture was stirred at 20° C. for 18 hr, then cooled to 0° C. and Sodium Borohydride (74.37 mg, 1.97 mmol, 69.25 μL) was added in one portion. The reaction mixture was allowed to warm to rt and stirred for 1 hr, then concentrated in vacuo. The residue was diluted with water (25 mL) and extracted with dichloromethane (40 mL). The organic layer was separated, dried over sodium sulfate and concentrated in vacuo to afford N-[[2-methyl-4-(pentafluoro-sulfanyl)phenyl]methyl]ethanamine (215 mg, crude).

LCMS (ESI): [M+H]⁺m/z: calcd 276.2; found 276.2; Rt=1.023 min.

### Step 7: The Synthesis of 2,2,2-Trifluoroethyl 2-[ethyl-[[2-methyl-4-(pentafluoro-sulfanyl)phenyl]methyl]amino]-2-oxo-acetate

2,2,2-Trifluoroethyl 2-chloro-2-oxo-acetate (207.06 mg, 1.09 mmol) was added dropwise to a solution of N-[[2-methyl-4-(pentafluoro-sulfanyl)phenyl]methyl]ethanamine (272 mg, 988.08 μmol) and triethylamine (119.98 mg, 1.19 mmol, 165.26 μL) in DCM (10 mL) at 0° C. After addition was complete, cooling bath was removed and resulting mixture was allowed to warm up to 20° C. and stirred for 16 hr. Then, it was washed with water, dried over Na₂SO₄ and concentrated under reduced pressure, affording 2,2,2-trifluoroethyl 2-[ethyl-[[2-methyl-4-(pentafluoro-sulfanyl)phenyl]methyl]amino]-2-oxo-acetate (0.38 g, crude) which was used in the next step without further purification.

LCMS (ESI): [M+H]⁺m/z: calcd 430.0; found 430.0; Rt=1.520 min.

### Step 8: The Synthesis of N′-Ethyl-N′-[[2-methyl-4-(pentafluoro-sulfanyl)phenyl]methyl]oxamide

A solution of 2,2,2-trifluoroethyl 2-[ethyl-[2-methyl-4-(pentafluoro-sulfanyl)phenyl]methyl]amino]-2-oxo-acetate (0.38 g, 885.11 μmol) in Methanol/NH₃ (5N)(5 mL) was stirred at 20° C. for 16 hr. The solvent was evaporated to dryness and then submitted to reverse phase HPLC (0-2-10 min 23-30-70 H₂O/MeOH; flow 30 mL/min ((loading pump 4 mL MeOH); column: XBridge BEH C18 100*19 mm, 5 microM) to obtain N′-ethyl-N′-[[2-methyl-4-(pentafluoro-sulfanyl)phenyl]methyl]oxamide (84 mg, 242.55 μmol, 27.40% yield).

LCMS (ESI): [M+H]⁺m/z: calcd 347.0; found 347.0; Rt=1.279 min.

### Step 9: The Synthesis of N-(4-Amino-2-tetrahydropyran-2-yl-pyrazolo[4,3-c]pyridin-7-yl)-N′-ethyl-N′-[[2-methyl-4-(pentafluoro-sulfanyl)phenyl]methyl]oxamide

N′-Ethyl-N′-[[2-methyl-4-(pentafluoro-sulfanyl)phenyl]methyl]oxamide (84 mg, 242.55 μmol), 7-bromo-2-tetrahydropyran-2-yl-pyrazolo[4,3-c]pyridin-4-amine (79.28 mg, 266.81 μmol), Copper (I) iodide (9.24 mg, 48.51 μmol, 1.64 μL), Cesium carbonate (158.06 mg, 485.11 μmol) and (1R,2R)—N1,N2-dimethylcyclohexane-1,2-diamine (41.40 mg, 291.06 μmol) were mixed in dioxane (4 mL) under argon, and then stirred overnight at 100° C. for 36 hr in vial. The reaction mixture was filtered and the filtrate was concentrated in vacuo to give N-(4-amino-2-tetrahydropyran-2-yl-pyrazolo[4,3-c]pyridin-7-yl)-N′-ethyl-N′-[[2-methyl-4-(pentafluoro-sulfanyl)phenyl]methyl]oxamide (0.3 g, crude).

LCMS (ESI): [M+H]⁺m/z: calcd 563.2; found 563.2; Rt=1.112 min.

### Step 10: The Synthesis of N-(4-Amino-1H-pyrazolo[4,3-c]pyridin-7-yl)-N′-ethyl-N′-[[2-methyl-4-(pentafluoro-sulfanyl)phenyl]methyl]oxamide (Compound 194)

To a solution of N-(4-amino-2-tetrahydropyran-2-yl-pyrazolo[4,3-c]pyridin-7-yl)-N′-ethyl-N′-[[2-methyl-4-(pentafluoro-sulfanyl)phenyl]methyl]oxamide (0.3 g, 202.65 μmol) in MeOH (4 mL) was added Hydrogen chloride solution 4.0M in dioxane (7.39 mg, 202.65 μmol, 1 mL) at 20° C. The resulting mixture was left to stirred for 16 hr. The resulting mixture was evaporated to dryness and then submitted to reverse phase HPLC (0-2-9 min Aug. 15, 1975 H₂O/ACN/0.1FA flow 30 mL/min ((loading pump 4 mL ACN); column: XBridge BEH C18 100*19 mm, 5 microM) to afford N-(4-amino-1H-pyrazolo[4,3-c]pyridin-7-yl)-N′-ethyl-N′-[2-methyl-4-(pentafluoro-sulfanyl)phenyl]methyl]oxamide (21 mg, 40.04 μmol, 19.76% yield, HCOOH).

¹H NMR (600 MHz, dmso) 8 0.93-1.21 (m, 3H), 2.17-2.43 (m, 3H), 3.23-3.56 (m, 2H), 4.28-4.92 (m, 2H), 6.49-7.07 (m, 2H), 7.20-7.83 (m, 4H), 8.14-8.29 (m, 1H), 9.53-10.50 (m, 1H), 12.70-13.41 (m, 1H).

LCMS (ESI): [M+H]⁺m/z: calcd 479.2; found 479.0; Rt=1.033 min.

### Example 131. Synthesis of N-(4-Amino-1H-pyrazolo[4,3-c]pyridin-7-yl)-N′-methyl-N′—[(1R)-1-[2-fluoro-4-(1,1,2,2,2-pentafluoroethyl)phenyl]ethyl]oxamide (Compound 188) and N-(4-Amino-1H-pyrazolo[4,3-c]pyridin-7-yl)-N′-methyl-N′—[(1S)-1-[2-fluoro-4-(1,1,2,2,2-pentafluoroethyl)phenyl]ethyl]oxamide (Compound 168)

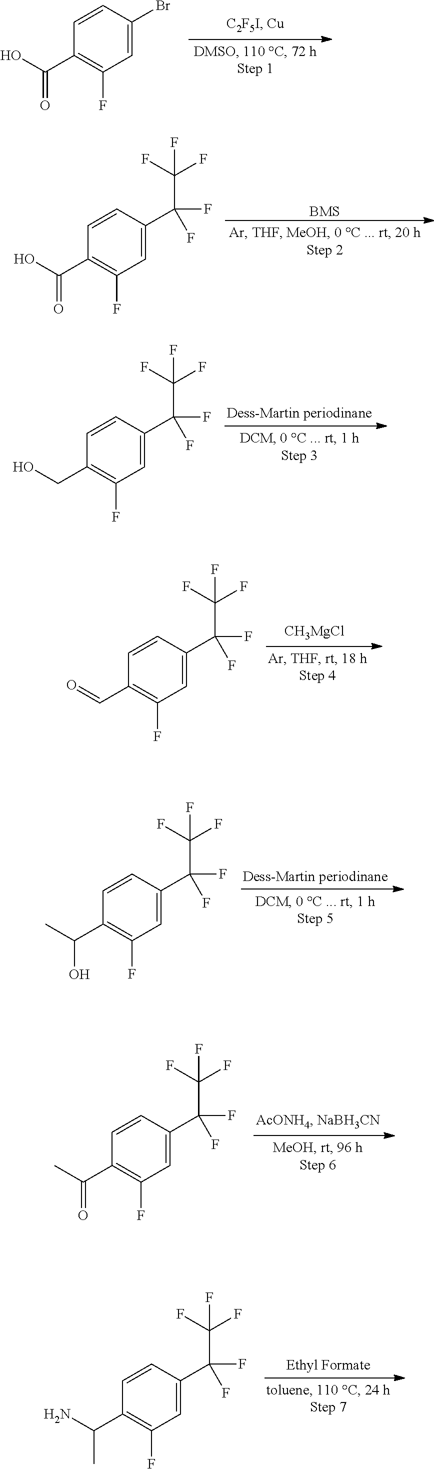

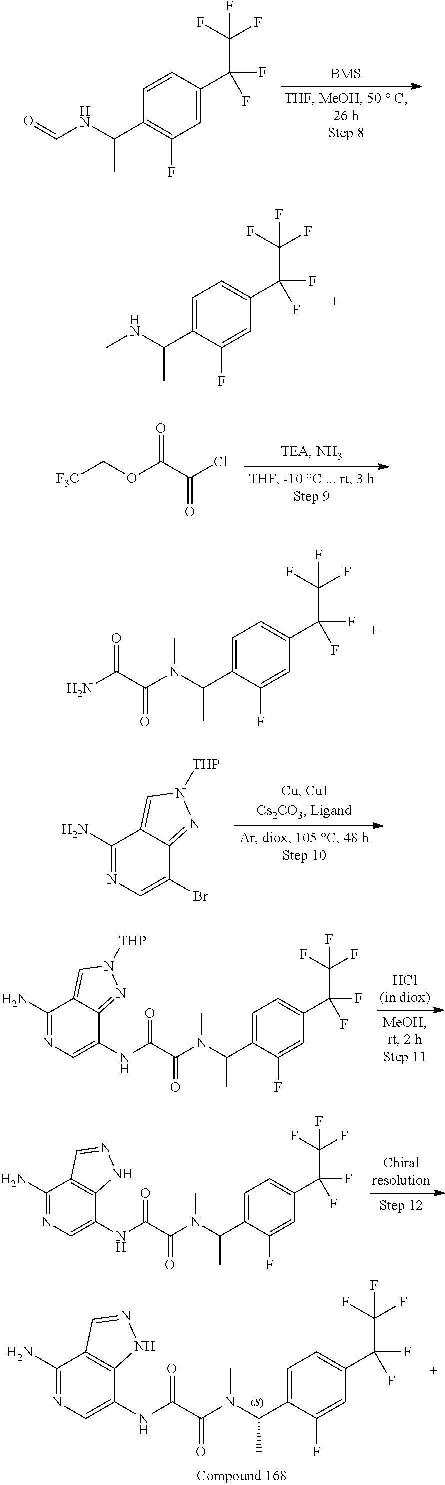

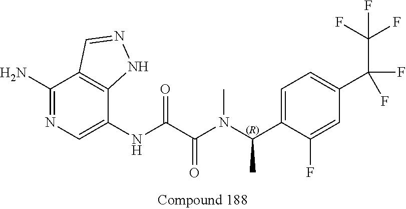

### Steps 1-6 were described for Compound 134, Compound 103

### Step 7: The Synthesis of N-[1-[2-Fluoro-4-(1,1,2,2,2-pentafluoroethyl)phenyl]ethyl]formamide

A mixture of 1-[2-fluoro-4-(1,1,2,2,2-pentafluoroethyl)phenyl]ethanamine (0.65 g, 2.53 mmol) and Ethyl Formate (1.87 g, 25.27 mmol, 2.03 mL) in toluene (18.98 mL) was stirred under reflux at 110° C. for 24 hr, then cooled down and concentrated in vacuo to afford crude N—[1-[2-fluoro-4-(1,1,2,2,2-pentafluoroethyl)phenyl]ethyl]formamide (0.75 g, 2.63 mmol, 104.05% yield) as light-brown gum.

LCMS (ESI): [M+H]⁺m/z: calcd 286.2; found 286.1; Rt=1.347 min.

### Step 8: The Synthesis of 1-[2-Fluoro-4-(1,1,2,2,2-pentafluoroethyl)phenyl]-N-methyl-ethanamine

Borane dimethyl sulfide complex (998.96 mg, 13.15 mmol, 1.25 mL) was added dropwise under argon to a cooled to 0° C. solution of N-[1-[2-fluoro-4-(1,1,2,2,2-pentafluoroethyl)phenyl]ethyl]formamide (750 mg, 2.63 mmol) in THF (25 mL). The resulting mixture was allowed to warm to 50° C. and stirred for 24 hr. The reaction mixture was again cooled to 0° C. and methanol (20 mL) was added dropwise over 0.5 hr. Then 2N aqueous hydrochloric acid (10 mL) was added slowly, and the resulting mixture was gradually warmed to 50° C. and stirred for 2 hr. The reaction mixture was cooled down and concentrated in vacuo, the residue was basified with 10% aqueous sodium hydroxide solution to pH 10-11 and extracted with MTBE (2*30 mL). The combined organic extracts were dried over sodium sulfate and concentrated in vacuo to afford crude 1-[2-fluoro-4-(1,1,2,2,2-pentafluoroethyl)phenyl]-N-methyl-ethanamine (600 mg, 2.21 mmol, 84.12% yield) as yellow oil, which was used directly in the next step.

LCMS (ESI): [M+H]⁺m/z: calcd 272.2; found 272.1; Rt=0.976 min.

### Step 9: The Synthesis of N′-[1-[2-Fluoro-4-(1,1,2,2,2-pentafluoroethyl)phenyl]ethyl]-N′-methyl-oxamide

2,2,2-Trifluoroethyl 2-chloro-2-oxo-acetate (547.91 mg, 2.88 mmol) was added slowly to a cooled to −10° C. mixture of 1-[2-fluoro-4-(1,1,2,2,2-pentafluoroethyl)phenyl]-N-methyl-ethanamine (600 mg, 2.21 mmol) and triethyl amine (1.10 g, 10.84 mmol, 1.51 mL) in THF (50 mL). The resulting mixture was allowed to warm to 25° C. and stirred for 2 hr. Then gaseous ammonia (37.68 mg, 2.21 mmol, 43.81 μL) was vigorously bubbled through it at 25° C. for 1 hr. The resulting mixture was filtered to remove ammonium chloride, the filtercake was washed with THF (2*20 mL) and discarded. The combined filtrate was concentrated in vacuo to afford crude N′-[1-[2-fluoro-4-(1,1,2,2,2-pentafluoroethyl)phenyl]ethyl]-N′-methyl-oxamide (760 mg, crude) as light-yellow solid, which was used directly in the next step.

### Step 10: The Synthesis of N-(4-Amino-2-tetrahydropyran-2-yl-pyrazolo[4,3-c]pyridin-7-yl)-N′-[1-[2-fluoro-4-(1,1,2,2,2-pentafluoroethyl)phenyl]ethyl]-N′-methyl-oxamide

A mixture of N′-[1-[2-fluoro-4-(1,1,2,2,2-pentafluoroethyl)phenyl]ethyl]-N′-methyl-oxamide (400 mg, 1.17 mmol), 7-bromo-2-tetrahydropyran-2-yl-pyrazolo[4,3-c]pyridin-4-amine (434.13 mg, 1.46 mmol), copper (5.20 mg, 81.90 μmol), Copper (I) iodide (162.03 mg, 850.76 μmol, 28.83 μL), cesium carbonate (571.22 mg, 1.75 mmol) and rac-(1R,2R)—N1,N2-dimethylcyclohexane-1,2-diamine (162.03 mg, 1.14 mmol) in 1,4-dioxane (7 mL) was stirred in a sealed vial under argon at 105° C. for 48 hr. The resulting mixture was cooled down and filtered. The filtercake was washed successively with THF (2*5 mL) and dichloromethane (3*5 mL). The combined filtrate was concentrated in vacuo to afford crude N-(4-amino-2-tetrahydropyran-2-yl-pyrazolo[4,3-c]pyridin-7-yl)-N′-[1-[2-fluoro-4-(1,1,2,2,2-pentafluoroethyl)phenyl]ethyl]-N′-methyl-oxamide (1.2 g, crude) as brown gum, which was used directly in the next step.

LCMS (ESI): [M+H]⁺m/z: calcd 559.2; found 559.2; Rt=3.511 min.

### Step 11: The Synthesis of N-(4-Amino-1H-pyrazolo[4,3-c]pyridin-7-yl)-N′-[1-[2-fluoro-4-(1,1,2,2,2-pentafluoroethyl)phenyl]ethyl]-N′-methyl-oxamide

Hydrogen chloride solution 4.0M in dioxane (7.40 g, 28.20 mmol, 7.05 mL, 13.9% purity) was added to a stirred solution of crude from previous step N-(4-amino-2-tetrahydropyran-2-yl-pyrazolo[4,3-c]pyridin-7-yl)-N′-[1-[2-fluoro-4-(1,1,2,2,2-pentafluoroethyl)phenyl]ethyl]-N′-methyl-oxamide (1.27 g, 2.27 mmol) in methanol (2.95 mL) at 25° C. The resulting solution was stirred at 25° C. for 2 hr, then concentrated to dryness in vacuo to afford crude product, which was purified by reverse phase HPLC (column: Agilent 5 PrepC18 100×30 mm 5 μm; mobile phase: 20-20-50% 0-1-5 min H₂O/MeCN/0.2% FA; flow: 30 mL/min; (loading pump 4 mL/min acetonitrile)) to afford N-(4-amino-1H-pyrazolo[4,3-c]pyridin-7-yl)-N′-[1-[2-fluoro-4-(1,1,2,2,2-pentafluoroethyl)phenyl]ethyl]-N′-methyl-oxamide (170 mg, 358.38 μmol, 15.78% yield) as light-brown solid.

LCMS (ESI): [M+H]⁺m/z: calcd 475.2; found 475.0; Rt=2.973 min.

### Step 12: The Synthesis of N-(4-Amino-1H-pyrazolo[4,3-c]pyridin-7-yl)-N′-methyl-N′—[(1R)-1-[2-fluoro-4-(1,1,2,2,2-pentafluoroethyl)phenyl]ethyl]oxamide (Compound 188) and N-(4-Amino-1H-pyrazolo[4,3-c]pyridin-7-yl)-N′-methyl-N′—[(1S)-1-[2-fluoro-4-(1,1,2,2,2-pentafluoroethyl)phenyl]ethyl]oxamide (Compound 168)

Racemic N-(4-amino-1H-pyrazolo[4,3-c]pyridin-7-yl)-N′-[1-[2-fluoro-4-(1,1,2,2,2-pentafluoroethyl)phenyl]ethyl]-N′-methyl-oxamide (170.00 mg, 358.38 μmol) was submitted to preparative chiral HPLC (Column: CHIRALPAK IC (250×30 mm, 10 mkm) Mobile Phase: Hexane (0.1% DEA): IPA: MeOH, 70:15:15; Flow Rate: 30 mL/min) to afford Compound 188-N-(4-amino-1H-pyrazolo[4,3-c]pyridin-7-yl)-N′-methyl-N′—[(1R)-1-[2-fluoro-4-(1,1,2,2,2-pentafluoroethyl)phenyl]ethyl]oxamide (69 mg, 145.46 μmol, 40.59% yield)(RetTime=14.636 min.) as light-yellow solid; and Compound 168-N-(4-amino-1H-pyrazolo[4,3-c]pyridin-7-yl)-N′-methyl-N′—[(1S)-1-[2-fluoro-4-(1,1,2,2,2-pentafluoroethyl)phenyl]ethyl]oxamide (66 mg, 139.14 μmol, 38.82% yield)(RetTime=20.346 min.) as light-yellow solid.

### Compound 188:

Yield: 69.0 mg (40.59%)

RT (Chiralpak IC (250×4.6 mm, 5 mkm); Hexane (0.1% EDA): IPA: MeOH, 70:15:15; Flow Rate: 0.6 mL/min)=14.926 min.

¹H NMR (600 MHz, dmso) δ 1.51-1.73 (m, 3H), 2.65-2.99 (m, 3H), 5.34-5.97 (m, 1H), 6.66 (s, 2H), 7.30-7.85 (m, 4H), 8.06-8.23 (m, 1H), 9.56-10.51 (m, 1H), 12.47-13.33 (m, 1H).

LCMS (ESI): [M+H]⁺m/z: calcd 475.2; found 475.4; Rt=1.838 min.

### Compound 168:

Yield: 66.0 mg (38.82%)

RT (Chiralpak IC (250×4.6 mm, 5 mkm); Hexane (0.1% EDA): IPA: MeOH, 70:15:15; Flow Rate: 0.6 mL/min)=20.134 min.

¹H NMR (600 MHz, dmso) 8 1.51-1.70 (m, 3H), 2.67-2.98 (m, 3H), 5.34-5.95 (m, 1H), 6.53-7.18 (m, 2H), 7.33-7.85 (m, 4H), 8.06-8.29 (m, 1H), 9.55-10.60 (m, 1H), 12.57-13.42 (m, 1H).

LCMS (ESI): [M+H]⁺m/z: calcd 475.2; found 475.4; Rt=1.839 min.

The absolute stereochemistry of the two compounds was independently confirmed.

### Example 132. Synthesis of The synthesis of N-(4-amino-1H-pyrazolo[4,3-c]pyridin-7-yl)-N′-benzyl-N′-[[2-(trifluoromethyl)phenyl]methyl]oxamide (Compound 95)

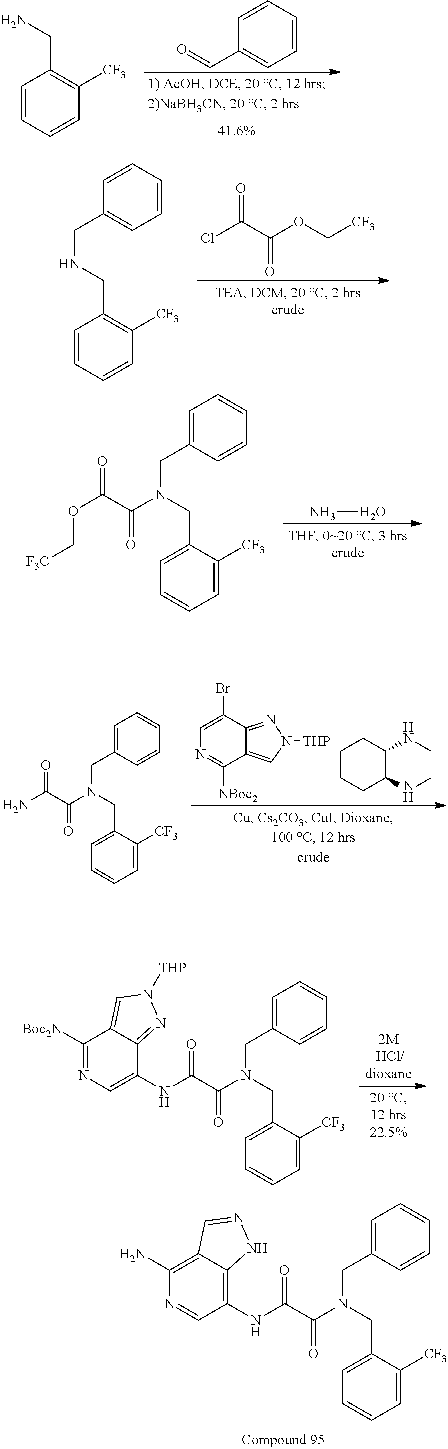

### Step 1: Synthesis of 1-phenyl-N-[[2-(trifluoromethyl)phenyl]methyl]methanamine

To a mixture of [2-(trifluoromethyl)phenyl]methanamine (990 mg, 5.65 mmol), benzaldehyde (500 mg, 4.71 mmol) in DCE (10 mL) was added acetic acid (283 mg, 4.71 mmol). The resulting mixture was stirred at 20° C. for 12 hours. The resulting mixture was added sodium; cyanoboranuide (444 mg, 7.07 mmol), and then stirred at 20° C. for 2 hours. The reaction was quenched by addition of saturated NH₄Cl aqueous solution (30 mL), and then extracted with EtOAc (50 mL×3). The combined organic layer was washed brine (50 mL), dried over anhydrous Na₂SO₄, filtered and concentrated under reduced pressure. The residue was purified by preparative HPLC (CP; Column: Boston Prime C18 150×30 mm×5 μm; Mobile phase A: H₂O with 10 mm NH₃H₂₀+NH₄HCO₃ (v %); Mobile phase B: ACN; Gradient: B from 60% to 80% in 10 min, hold 100% B for 2 min; Flow Rate: 30 mL/min; Column Temperature: 30° C.; Wavelength: 220 nm, 254 nm) to afford 1-phenyl-N-[[2-(trifluoromethyl)phenyl]methyl]methanamine (520 mg, 41.6% yield) as light-yellow oil.

¹H NMR (400 MHz, DMSO-d₆) δ ppm 7.86 (br d, J=7.3 Hz, 1H), 7.61-7.72 (m, 2H), 7.18-7.50 (m, 6H), 3.85 (br s, 2H), 3.72 (s, 2H), 2.74 (br s, 1H), 2.57-2.59 (m, 1H); LCMS (ESI) [M+H]⁺m/z: calcd 266.1, found 266.2.

### Step 2: Synthesis of N′-benzyl-N′-[[2-(trifluoromethyl)phenyl]methyl]oxamide

To a mixture of 1-phenyl-N-[2-(trifluoromethyl)phenyl]methyl]methanamine (470 mg, 1.77 mmol), TEA (273 mg, 2.70 mmol) in DCM (10 mL) at 0° C. was added 2,2,2-trifluoroethyl 2-chloro-2-oxo-acetate (675 mg, 3.54 mmol). and then the resulting mixture stirred at 20° C. for 2 hours. The reaction mixture was quenched by addition water (30 mL) at 20° C., extracted with DCM (50 mL×3). The combined organic layers were washed with brine (30 mL×3), dried over anhydrous Na₂SO₄, filtered and concentrated under reduced pressure to give a residue to afford 2,2,2-trifluoroethyl 2-[benzyl-[[2-(trifluoromethyl)phenyl]methyl]amino]-2-oxo-acetate (800 mg, crude) as yellow oil. LCMS (ESI) [M+H]⁺m/z: calcd 420.1, found 420.2.

### Step 3: Synthesis of N′-benzyl-N′-[[2-(trifluoromethyl)phenyl]methyl]oxamide

To a mixture of 2,2,2-trifluoroethyl 2-[benzyl-[[2-(trifluoromethyl)phenyl]methyl]amino]-2-oxo-acetate (800 mg, 1.91 mmol) in THF (8 mL) at 0° C. was added NH3-H₂O(670 mg, 19.1 mmol) the mixture stirred at 0° C. for 15 min, then the resulting mixture stirred at 20° C. for 3 hours. The reaction mixture was quenched by addition water (30 mL) at 20° C., extracted with DCM (50 mL×3). The combined organic layers were washed with brine (30 mL×3), dried over anhydrous Na₂SO₄, filtered and concentrated under reduced pressure to afford N′-benzyl-N′-[[2-(trifluoromethyl)phenyl]methyl]oxamide (700 mg, crude) as yellow oil. LCMS (ESI) [M+H]⁺m/z: calcd 337.1, found 337.2.

### Step 4: Synthesis of tert-butyl N-[7-[2-[benzyl-[[2-(trifluoromethyl)phenyl]methyl]amino]-2-oxo-acetyl]amino]-2-tetrahydropyran-2-yl-pyrazolo[4,3-c]pyridin-4-yl]-N-tert-butoxycarbonyl-carbamate

To a mixture of N′-benzyl-N′-[[2-(trifluoromethyl)phenyl]methyl]oxamide (650 mg, 1.93 mmol), tert-butyl N-(7-bromo-2-tetrahydropyran-2-yl-pyrazolo[4,3-c]pyridin-4-yl)-N-tert-butoxycarbonyl-carbamate (780 mg, 1.57 mmol), rac-(1S,2S)-N1,N2-dimethylcyclohexane-1,2-diamine (337 mg, 2.37 mmol), Cs₂CO₃ (1.02 g, 3.14 mmol) in dioxane (15 mL) at 20° C. was added Cu(103 mg, 1.62 mmol) and CuI (296 mg, 1.55 mmol), the resulting mixture stirred at 100° C. for 12 hours. The reaction mixture was quenched by addition water (30 mL) at 20° C., extracted with EtOAc (30 mL×3). The combined organic layers were washed with brine (30 mL×3), dried over anhydrous Na₂SO₄, filtered and concentrated under reduced pressure. The residue was purified by flash silica gel chromatography (ISCO®; 20g SepaFlash® Silica Flash Column, Petroleum ether/EtOAc with EtOAc from 0~30%, flow rate: 35 mL/min, 254 nm) to afford tert-butyl N-[7-[2-[benzyl-[[2-(trifluoromethyl)phenyl]methyl]amino]-2-oxo-acetyl]amino]-2-tetrahydropyran-2-yl-pyrazolo[4,3-c]pyridin-4-yl]-N-tert-butoxycarbonyl-carbamate (400 mg, crude) as yellow oil.

¹H NMR (400 MHz, dmso-d6) δ ppm 11.27 (s, 1H), 11.06 (s, 1H), 8.82 (s, 1H), 8.79 (s, 1H), 8.58 (s, 1H), 8.44 (s, 1H), 7.65-7.76 (m, 4H), 7.48-7.58 (m, 3H), 7.39-7.44 (m, 2H), 7.27-7.37 (m, 6H), 7.19 (br d, J=7.0 Hz, 1H), 5.82 (dd, J=9.8, 2.51 Hz, 1H), 5.74-5.79 (m, 1H), 4.90 (s, 1H), 4.79 (s, 1H), 4.64 (s, 1H), 4.55-4.60 (m, 1H), 4.52 (s, 1H), 4.46-4.54 (m, 1H), 4.47 (s, 1H), 4.34 (br s, 1H), 3.64-3.75 (m, 2H), 1.98 (s, 4H), 1.35-1.38 (m, 18H); LCMS (ESI) [M+H]⁺m/z: calcd 753.3, found 753.3.

### Step 5: Synthesis of N-(4-amino-1H-pyrazolo[4,3-c]pyridin-7-yl)-N′-benzyl-N′-[[2-(trifluoromethyl)phenylmethyl]oxamide (Compound 95)

To a mixture of tert-butyl N-[7-[2-[benzyl-[2-(trifluoromethyl)phenyl]methyl]amino]-2-oxo-acetyl]amino]-2-tetrahydropyran-2-yl-pyrazolo[4,3-c]pyridin-4-yl]-N-tert-butoxycarbonyl-carbamate (350 mg, 0.465 mmol) in dioxane (2 mL) was added 2M HCl in dioxane (2 mL, 4.0 mmol). The resulting mixture was stirred at 20° C. for 12 hours. The mixture was adjusted pH~8 with saturated NaHCO₃ aqueous solution. The reaction mixture was concentrated under reduced pressure. The residue was purified by preparative HPLC (Instrument: Gilson GX-281 Liquid Handler, Gilson 322 Pump, Gilson 156 UV Detector; Column: 2_Phenomenex Gemini C18 75 x 40 mm×3 μm; Mobile phase A: H₂O with10 mm NH3H₂O+NH₄HCO₃ (v %); Mobile phase B: ACN; Gradient: B from 38% to 68% in 7.8 min, hold 100% B for 3 min, Flow Rate: 30 mL/min; Column Temperature: 30° C.; Wavelength: 220 nm, 254 nm) to afford N-(4-amino-1H-pyrazolo[4,3-c]pyridin-7-yl)-N′-benzyl-N′-[2-(trifluoromethyl)phenyl]methyl]oxamide (49 mg, 22.5% yield) as a white solid.

Compound 95: 1H NMR (400 MHz, dmso-d6) δ ppm 12.32-13.22 (m, 1H), 10.44 (br s, 1H), 8.14-8.29 (m, 1H), 7.44-7.86 (m, 4H), 7.03-7.42 (m, 5H), 6.44 (br d, J=11.8 Hz, 2H), 5.04 (s, 1H), 4.87 (s, 1H), 4.78-5.16 (m, 1H), 4.71 (s, 1H), 4.59 (s, 1H); LCMS (ESI) [M+H]⁺m/z: calcd 469.1, found 469.1; HPLC: 99.49% @220 nm, 100.00% @254 nm.

### Example 133. Synthesis of N-(4-amino-1H-pyrazolo[4,3-c]pyridin-7-yl)-N′-[(2-fluorophenyl)methyl]-N′-[5-(trifluoromethyl)-2-pyridyl]methyl]oxamide (Compound 111)

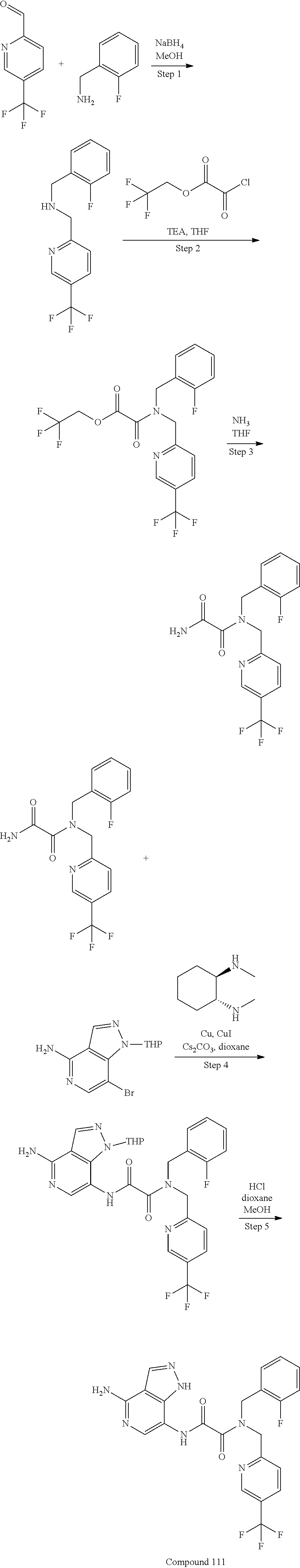

### Step 1:1-(2-fluorophenyl)-N-[5-(trifluoromethyl)-2-pyridyl]methyl]methanamine

A solution of 5-(trifluoromethyl)pyridine-2-carbaldehyde (0.7 g, 4.00 mmol) and (2-fluorophenyl)methanamine (500.26 mg, 4.00 mmol, 455.20 μL) in MeOH (20.45 mL) was stirred at 20° C. for 12 hr. To this solution, Sodium Borohydride (166.35 mg, 4.40 mmol, 154.89 μL) was added and the resulting mixture was stirred for 5 hr. The solvent was removed in vacuo, the residue was taken up with water (40 mL) and extracted with DCM (3×20 mL). The combined organic layer was washed with brine (10 mL), dried over anhydrous sodium sulfate and evaporated to obtain 1-(2-fluorophenyl)-N-[[5-(trifluoromethyl)-2-pyridyl]methyl]methanamine (1 g, 3.52 mmol, 88.00% yield) as a brown oil.

LCMS (ESI): [M+H]⁺m/z: calcd 285.1; found 286.2; Rt=2.383 min.

### Step 2:2,2,2-trifluoroethyl 2-[(2-fluorophenyl)methyl-[[5-(trifluoromethyl)-2-pyridyl]methyl]amino]-2-oxo-acetate

To a solution of 1-(2-fluorophenyl)-N-[5-(trifluoromethyl)-2-pyridyl]methyl]methanamine (1 g, 3.52 mmol) and TEA (533.98 mg, 5.28 mmol, 735.51 μL) in THF (30 mL) was added 2,2,2-trifluoroethyl 2-chloro-2-oxo-acetate (804.24 mg, 4.22 mmol) dropwise at 0° C. under argon. The reaction mixture was then stirred for 8 hr at r.t., then evaporated in vacuo to give 2,2,2-trifluoroethyl 2-[(2-fluorophenyl)methyl-[5-(trifluoromethyl)-2-pyridyl]methyl]amino]-2-oxo-acetate (1.25 g, 2.85 mmol, 81.07% yield) as a light-yellow oil.

LCMS (ESI): [M+H]⁺m/z: calcd 439.09; found 439.0; Rt=3.898 min.

### Step 3: N′-[(2-fluorophenyl)methyl]-N′-[[5-(trifluoromethyl)-2-pyridyl]methyl]oxamide

2,2,2-trifluoroethyl 2-[(2-fluorophenyl)methyl-[5-(trifluoromethyl)-2-pyridyl]methyl]amino]-2-oxo-acetate (1.25 g, 2.85 mmol) was dissolved in THF (40 mL) and was blow ammonium (1.03 g, 57.04 mmol). The resulting solution was stirred at 0° C. for 5 hr. The resulting mixture was evaporated in vacuo and residue was triturated with THF (2×20 mL), filtered and combined organic layers were evaporated in vacuo to leave 1 g of crude product which was purified by column chromatography on silica gel using CHCl₃/CH₃CN gradient (10-100% CH₃CN) to afford N′-[(2-fluorophenyl)methyl]-N′-[5-(trifluoromethyl)-2-pyridyl]methyl]oxamide (0.5 g, 1.41 mmol, 49.35% yield) as a light-yellow oil.

LCMS (ESI): [M+H]⁺m/z: calcd 356.1; found 356.2; Rt=1.034 min.

### Step 4: N-(4-amino-1-tetrahydropyran-2-yl-pyrazolo[4,3-c]pyridin-7-yl)-N′-[(2-fluorophenyl)methyl]-N′-[[5-(trifluoromethyl)-2-pyridyl]methyl]oxamide

7-bromo-1-tetrahydropyran-2-yl-pyrazolo[4,3-c]pyridin-4-amine (326.18 mg, 1.10 mmol), N′-[(2-fluorophenyl)methyl]-N′-[5-(trifluoromethyl)-2-pyridyl]methyl]oxamide (0.3 g, 844.39 μmol), Cu(13.42 mg, 211.26 μmol), CuI (0.15 g, 787.61 μmol, 26.69 μL), cesium carbonate (412.68 mg, 1.27 mmol) and (1R,2R)—N1,N2-dimethylcyclohexane-1,2-diamine (0.15 g, 1.05 mmol) were mixed in dioxane (6.00 mL), purged with Ar for 15 minutes and then heated in the sealed tube at 105° C. for 48 hr. Final mixture was filtered and dioxane was evaporated in vacuo. The crude product 0.6 g was purified by RP-HPLC (column: XBridge C18 5 μm 130A; 50-50-90% 0-1.3-5.3 min H₂O/CH₃OH/0.1% NH₄OH, flow: 30 mL/min) to give N-(4-amino-1-tetrahydropyran-2-yl-pyrazolo[4,3-c]pyridin-7-yl)-N′-[(2-fluorophenyl)methyl]-N′-[5-(trifluoromethyl)-2-pyridyl]methyl]oxamide (294.10 mg, 514.59 μmol, 60.94% yield) as a brown solid.

LCMS (ESI): [M+H]⁺m/z: calcd 572.23; found 572.2; Rt=3.260 min.

### Step 5: The synthesis of N-(4-amino-1H-pyrazolo[4,3-c]pyridin-7-yl)-N′-[(2-fluorophenyl)methyl]-N′-[[5-(trifluoromethyl)-2-pyridyl]methyl]oxamide

Hydrogen chloride solution 4.0M in dioxane (2.40 g, 65.83 mmol, 3 mL) was added to a solution of N-(4-amino-1-tetrahydropyran-2-yl-pyrazolo[4,3-c]pyridin-7-yl)-N′-[(2-fluorophenyl)methyl]-N′-[[5-(trifluoromethyl)-2-pyridyl]methyl]oxamide (363.39 mg, 635.83 μmol) in MeOH (3 mL). The reaction mixture was stirred at 20° C. for 24 hr, then evaporated. The residue was purified by RP-HPLC (column: XBridge BEH18 SMB100-BT 100×19 mm; 45-45-85% 0-1.5-5 min H₂O/CH₃OH/0.1% NH₄OH, flow: 3 0 mL/min) to give N-(4-amino-1H-pyrazolo[4,3-c]pyridin-7-yl)-N′-[(2-fluorophenyl)methyl]-N′-[5-(trifluoromethyl)-2-pyridyl]methyl]oxamide (0.0631 g, 129.46 μmol, 20.36% yield) as a light-yellow solid.

¹H NMR (600 MHz, DMSO-d₆) δ 4.24-5.14 (m, 4H), 6.60-6.99 (m, 2H), 6.99-7.22 (m, 2H), 7.27-7.36 (m, 1H), 7.37-7.49 (m, 1H), 7.50-7.69 (m, 2H), 8.11-8.23 (m, 2H), 8.61-8.92 (m, 1H), 9.75-10.64 (m, 1H), 12.62-13.41 (m, 1H).

LCMS (ESI): [M+H]⁺m/z: calcd 488.16; found 488.2; Rt=2.439 min.

### Example 134. Synthesis of N-(4-amino-2H-pyrazolo[4,3-c]pyridin-7-yl)-N′-(chroman-5-ylmethyl)-N′-(2-pyridylmethyl)oxamide (Compound 92)

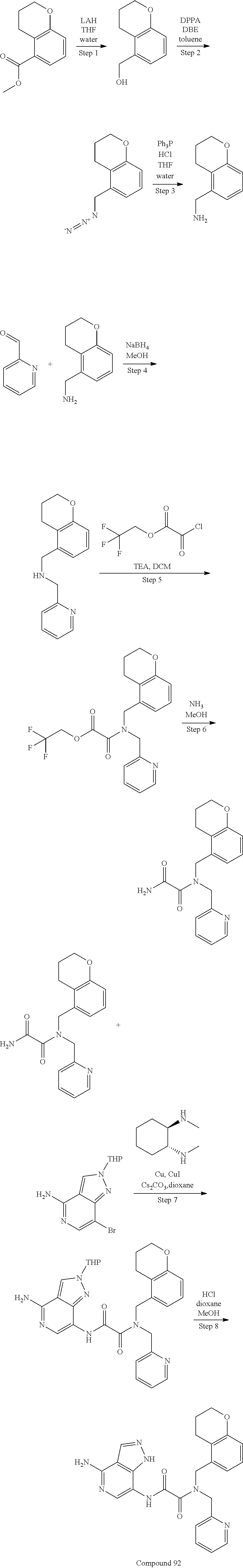

### Step 1: chroman-5-ylmethanol

Methyl chromane-5-carboxylate (900 mg, 4.68 mmol) was added dropwise to the ice-cooled solution of LAH (266.57 mg, 7.02 mmol) in Tetrahydrofuran (30 mL) under argon. After addition was complete, cooling bath was removed and resulting reaction mixture was stirred at 20° C. for 2 hr. Then, reaction was quenched by dropwise addition of Water (1.4 mL) dissolved in Tetrahydrofuran (5 mL). After that, reaction mixture was filtered and concentrated under reduced pressure, leaving chroman-5-ylmethanol (780 mg, crude) as a light-yellow oil.

LCMS (ESI): [M-OH]⁺m/z: calcd 147.07; found 147.2; Rt=0.939 min.

### Step 2:5-(azidomethyl)chromane

Diphenylphosphoryl azide (1.57 g, 5.70 mmol, 1.23 mL) was added to the solution of chroman-5-ylmethanol (780 mg, 4.75 mmol) and DBU (1.01 g, 6.65 mmol, 994.53 μL) in Toluene (25 mL). The resulting reaction mixture was stirred at 20° C. for 16 hr. Then, it was diluted with MTBE (25 mL) and washed successively with water (25 mL) and 10% aq. NH4Cl solution (25 mL). Organic layer was dried over anhydrous sodium sulfate and concentrated under reduced pressure to approximately 10 mL volume. The resulting solution of 5-(azidomethyl) chromane (0.898 g, 4.75 mmol, 99.91% yield) was used in the next step as is.

### Step 3: chroman-5-ylmethanamine

Triphenylphosphine (1.87 g, 7.13 mmol) was added to the solution of 5-(azidomethyl) chromane (0.9 g, 4.76 mmol) in Tetrahydrofuran (19.68 mL) and Water (19.68 mL). The resulting reaction mixture was stirred at 50° C. for 16 hr. Then, it was acidified with Hydrochloric acid, 36% w/w aq. soln. (400.00 mg, 10.97 mmol, 0.5 mL) and volatiles were removed under reduced pressure. The residue was partitioned between water (50 mL) and DCM (20 mL) and organic layer was separated and discarded. Aqueous layer was basified with K₂CO₃ to pH~10-11 and extracted with DCM (2×20 mL). Combined DCM solutions were dried over K₂CO₃ and evaporated under reduced pressure, leaving chroman-5-ylmethanamine (600 mg, 3.68 mmol, 77.29% yield) as a light-brown oil.

LCMS (ESI): [M+H]⁺m/z: calcd 164.11; found 164.0; Rt=0.386 min.

### Step 4:1-chroman-5-yl-N-(2-pyridylmethyl)methanamine

Pyridine-2-carbaldehyde (433.12 mg, 4.04 mmol, 385.34 μL) was added to the solution of chroman-5-ylmethanamine (600 mg, 3.68 mmol) in Methanol (20 mL). The resulting solution was stirred at 25° C. for 16 hr. Then, Sodium Borohydride (139.08 mg, 3.68 mmol, 129.49 μL) was added and stirring was continued for 1 hour. After that, volatiles were removed under reduced pressure and residue was partitioned between 10% aq. K₂CO₃ solution (15 mL) and DCM (25 mL). Organic layer was separated, dried over K₂CO₃ and evaporated under reduced pressure, affording 1-chroman-5-yl-N-(2-pyridylmethyl)methanamine (960 mg, crude) as a yellow oil.

LCMS (ESI): [M+H]⁺m/z: calcd 255.15; found 255.2; Rt=0.862 min.

### Step 5:2,2,2-trifluoroethyl 2-[chroman-5-ylmethyl(2-pyridylmethyl)amino]-2-oxo-acetate

2,2,2-trifluoroethyl 2-chloro-2-oxo-acetate (934.82 mg, 4.91 mmol) was added dropwise to the solution of 1-chroman-5-yl-N-(2-pyridylmethyl)methanamine (960 mg, 3.77 mmol) and Triethylamine (763.92 mg, 7.55 mmol, 1.05 mL) in Dichloromethane (30 mL) at 0° C. After addition was complete, cooling bath was removed and resulting mixture was allowed to warm up to 20° C. and stirred for 2 hr. Then, 10% aq. NaHCO₃ solution (20 mL) was added and stirring was continued for 5 min. After that, organic layer was separated, dried over K₂CO₃ and concentrated under reduced pressure, affording 2,2,2-trifluoroethyl 2-[chroman-5-ylmethyl(2-pyridylmethyl)amino]-2-oxo-acetate (1.54 g, 3.77 mmol, 100.00% yield) as a yellow oil.

LCMS (ESI): [M+H]⁺m/z: calcd 409.14; found 409.0; Rt=1.344 min.

### Step 6: N′-(chroman-5-ylmethyl)-N′-(2-pyridylmethyl)oxamide

2,2,2-trifluoroethyl 2-[chroman-5-ylmethyl(2-pyridylmethyl)amino]-2-oxo-acetate (1.54 g, 3.77 mmol) was dissolved in Ammonia (7N in methanol, 15,3% w/w)(7.79 g, 69.98 mmol, 10 mL, 15.3% purity). The resulting mixture was stirred at 25° C. for 16 hr. Then, volatiles were removed under reduced pressure, leaving N′-(chroman-5-ylmethyl)-N′-(2-pyridylmethyl)oxamide (1.24 g, crude) as a yellow solid.

LCMS (ESI): [M+H]⁺m/z: calcd 326.15; found 326.0; Rt=0.940 min.

### Step 7: N-(4-amino-2-tetrahydropyran-2-yl-pyrazolo[4,3-c]pyridin-7-yl)-N′-(chroman-5-ylmethyl)-N′-(2-pyridylmethyl)oxamide

N′-(chroman-5-ylmethyl)-N′-(2-pyridylmethyl)oxamide (228 mg, 700.76 μmol), 7-bromo-2-tetrahydropyran-2-yl-pyrazolo[4,3-c]pyridin-4-amine (249.88 mg, 840.91 μmol), Copper (4.45 mg, 70.08 μmol), Copper (I) iodide (66.73 mg, 350.38 μmol, 11.87 μL), (S,S)-(+)—N,N′-Dimethyl-1,2-cyclohexanediamine (49.84 mg, 350.38 μmol) and Potassium Carbonate (193.70 mg, 1.40 mmol, 84.58 μL) were mixed together in Dioxane (5 mL). The reaction flask was purged with argon and resulting mixture was stirred at 100° C. for 20 hr under inert atmosphere. Then, it was diluted with DCM (15 mL) and filtered. Filtrate was concentrated under reduced pressure, leaving N-(4-amino-2-tetrahydropyran-2-yl-pyrazolo[4,3-c]pyridin-7-yl)-N′-(chroman-5-ylmethyl)-N′-(2-pyridylmethyl)oxamide (560 mg, crude) as a brown gum.

LCMS (ESI): [M+H]⁺m/z: calcd 542.25; found 542.2; Rt=0.904 min.

### Step 8: The synthesis of N-(4-amino-2H-pyrazolo[4,3-c]pyridin-7-yl)-N′-(chroman-5-ylmethyl)-N′-(2-pyridylmethyl)oxamide

Hydrogen chloride solution 4.0M in dioxane (1.01 g, 2.77 mmol, 1 mL, 10% purity) was added to the solution of N-(4-amino-2-tetrahydropyran-2-yl-pyrazolo[4,3-c]pyridin-7-yl)-N′-(chroman-5-ylmethyl)-N′-(2-pyridylmethyl)oxamide (560 mg, 516.99 μmol) in Methanol (3 mL). The resulting mixture was stirred at 25° C. for 3 hr. Then, volatiles were removed under reduced pressure and residue was subjected to HPLC (10-35% 0-5 min H₂O/ACN/0.1% FA, flow: 30 mL/min, column: Chromatorex 18 SMB100-5T 100×19 mm 5 μm), affording N-(4-amino-2H-pyrazolo[4,3-c]pyridin-7-yl)-N′-(chroman-5-ylmethyl)-N′-(2-pyridylmethyl)oxamide (200 mg, 377.07 μmol, 72.94% yield, 2 HCl). ¹H NMR (600 MHz, DMSO-d₆) § 1.64-1.98 (m, 2H), 2.52-2.59 (m, 2H), 3.90-4.07 (m, 2H), 4.16-4.90 (m, 4H), 6.49-6.75 (m, 3H), 6.79-7.38 (m, 3H), 7.41-7.67 (m, 1H), 7.70-7.82 (m, 1H), 8.06-8.23 (m, 2H), 8.26-8.55 (m, 1H), 9.67-10.64 (m, 1H), 12.16-13.71 (m, 1H).

LCMS (ESI): [M+H]⁺m/z: calcd 458.21; found 459.2; Rt=2.268 min.

### Example 135. Synthesis of N-(4-amino-1H-pyrazolo[4,3-c]pyridin-7-yl)-N′-[(2-chlorophenyl)methyl]-N′-[5-(trifluoromethyl)-2-pyridyl]methyl]oxamide (Compound 182)

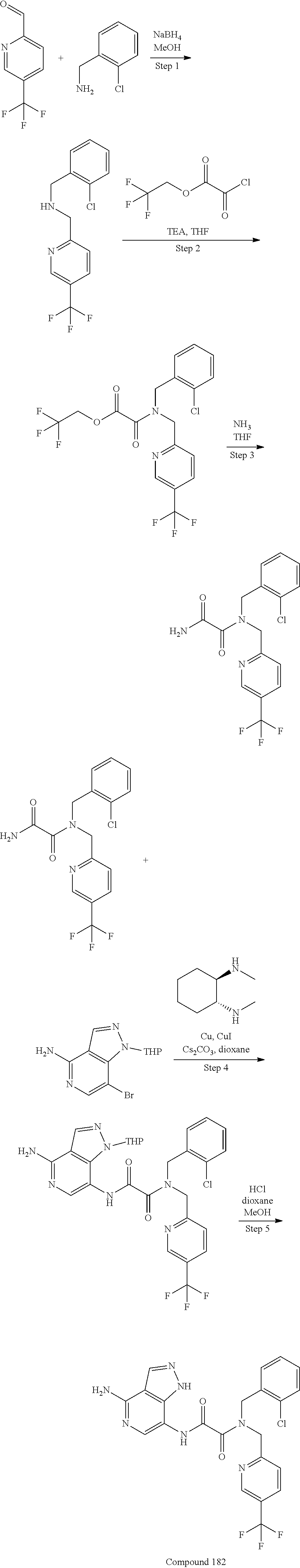

### Step 1:1-(2-chlorophenyl)-N-[[5-(trifluoromethyl)-2-pyridyl]methyl]methanamine

A solution of 5-(trifluoromethyl)pyridine-2-carbaldehyde (0.7 g, 4.00 mmol) and (2-chlorophenyl)methanamine (566.04 mg, 4.00 mmol, 483.80 μL) in MeOH (20 mL) was stirred at 20° C. for 12 hr. To this solution, Sodium Borohydride (166.35 mg, 4.40 mmol, 154.89 μL) was added and the resulting mixture was stirred for 5 hr. The solvent was removed in vacuo, the residue was taken up with water (40 mL) and extracted with DCM (3×20 mL). The combined organic layer was washed with brine (10 mL), dried over anhydrous sodium sulfate and evaporated to obtain 1-(2-chlorophenyl)-N-[5-(trifluoromethyl)-2-pyridyl]methyl]methanamine (1 g, 3.33 mmol, 83.19% yield) as a brown oil.

LCMS (ESI): [M+H]⁺m/z: calcd 301.07; found 301.2; Rt=2.476 min.

### Step 2:2,2,2-trifluoroethyl 2-[(2-chlorophenyl)methyl-[[5-(trifluoromethyl)-2-pyridyl]methyl]amino]-2-oxo-acetate

To a solution of 1-(2-chlorophenyl)-N-[5-(trifluoromethyl)-2-pyridyl]methyl]methanamine (1 g, 3.33 mmol) and TEA (504.76 mg, 4.99 mmol, 695.26 μL) in THF (30.04 mL) was added 2,2,2-trifluoroethyl 2-chloro-2-oxo-acetate (760.23 mg, 3.99 mmol) dropwise at 0° C. under argon. The reaction mixture was then stirred for 8 hr at r.t., then evaporated in vacuo to give 2,2,2-trifluoroethyl 2-[(2-chlorophenyl)methyl-[5-(trifluoromethyl)-2-pyridyl]methyl]amino]-2-oxo-acetate (1.3 g, 2.86 mmol, 85.96% yield) as a light-yellow oil.

### Step 3: N′-[(2-chlorophenyl)methyl]-N′-[[5-(trifluoromethyl)-2-pyridyl]methyl]oxamide

2,2,2-trifluoroethyl 2-[(2-chlorophenyl)methyl-[[5-(trifluoromethyl)-2-pyridyl]methyl]amino]-2-oxo-acetate (1.3 g, 2.86 mmol) was dissolved in THF (20 mL) and was blow ammonium (1.03 g, 57.17 mmol). The resulting solution was stirred at 20° C. for 5 hr. The resulting mixture was evaporated in vacuo and residue was triturated with THF (2×20 mL), filtered and combined organic layers were evaporated in vacuo to leave 1 g of crude product which was purification by column chromatography on silica gel using CHCl₃/CH₃CN gradient (10-100% CH₃CN) to afford N′-[(2-chlorophenyl)methyl]-N′-[[5-(trifluoromethyl)-2-pyridyl]methyl]oxamide (0.5 g, 1.35 mmol, 47.05% yield) as a brown oil.

LCMS (ESI): [M+H]⁺m/z: calcd 372.07; found 372.0; Rt=1.143 min.

### Step 4: N-(4-amino-1-tetrahydropyran-2-yl-pyrazolo[4,3-c]pyridin-7-yl)-N′-[(2-chlorophenyl)methyl]-N′-[5-(trifluoromethyl)-2-pyridyl]methyl]oxamide

7-bromo-1-tetrahydropyran-2-yl-pyrazolo[4,3-c]pyridin-4-amine (311.75 mg, 1.05 mmol), N′-[(2-chlorophenyl)methyl]-N′-[5-(trifluoromethyl)-2-pyridyl]methyl]oxamide (0.3 g, 807.01 μmol), Cu(1.54 mg, 24.21 μmol), CuI (0.15 g, 787.61 μmol, 26.69 μL), cesium carbonate (394.41 mg, 1.21 mmol) and (1R,2R)—N1,N2-dimethylcyclohexane-1,2-diamine (0.15 g, 1.05 mmol) were mixed in dioxane (6.00 mL), purged with Ar for 15 minutes and then heated in the sealed tube at 105° C. for 48 hr. Final mixture was filtered and dioxane was evaporated in vacuo. The crude product 0.6 g was purified by RP-HPLC (column: XBridge C18 5 μm 130A; 65-65-90% 0-2-5 min H₂O/CH₃OH/0.1% NH₄OH, flow: 30 mL/min) to give N-(4-amino-1-tetrahydropyran-2-yl-pyrazolo[4,3-c]pyridin-7-yl)-N′-[(2-chlorophenyl)methyl]-N′-[[5-(trifluoromethyl)-2-pyridyl]methyl]oxamide (0.1817 g, 309.02 μmol, 38.29% yield) as a brown solid.

LCMS (ESI): [M+H]⁺m/z: calcd 588.2; found 588.2; Rt=2.556 min.

### Step 5: The synthesis of N-(4-amino-1H-pyrazolo[4,3-c]pyridin-7-yl)-N′-[(2-chlorophenyl)methyl]-N′-[[5-(trifluoromethyl)-2-pyridyl]methyl]oxamide

Hydrogen chloride solution 4.0M in dioxane (9.28 g, 254.56 mmol, 11.60 mL) was added to a solution of N-(4-amino-1-tetrahydropyran-2-yl-pyrazolo[4,3-c]pyridin-7-yl)-N′-[(2-chlorophenyl)methyl]-N′-[5-(trifluoromethyl)-2-pyridyl]methyl]oxamide (0.1817 g, 309.02 μmol) in MeOH (13.39 mL). The reaction mixture was stirred at 20° C. for 24 hr, then evaporated. The residue was purified by RP-HPLC (column: XBridge BEH18 SMB100-BT 100×19 mm; 65-65-90% 0-2-5 min H₂O/CH₃OH/0.1% NH₄OH, flow: 30 mL/min) to give N-(4-amino-1H-pyrazolo[4,3-c]pyridin-7-yl)-N′-[(2-chlorophenyl)methyl]-N′-[5-(trifluoromethyl)-2-pyridyl]methyl]oxamide (0.0589 g, 116.90 μmol, 37.83% yield) as a light-yellow solid.

¹H NMR (600 MHz, DMSO-d₆) δ 4.59-4.79 (m, 2H), 4.85-5.21 (m, 2H), 6.51-7.08 (m, 2H), 7.26-7.68 (m, 6H), 8.08-8.33 (m, 2H), 8.79-8.96 (m, 1H), 9.76-10.69 (m, 1H), 12.56-13.51 (m, 1H).

LCMS (ESI): [M+H]⁺m/z: calcd 504.13; found 504.0; Rt=2.668 min.

### Example 136. Synthesis of N-(4-amino-1H-pyrazolo[4,3-c]pyridin-7-yl)-N′-[[2-(trifluoromethyl)phenylmethyl]-N′-[[5-(trifluoromethyl)-2-pyridyl]methyl]oxamide (Compound 163)

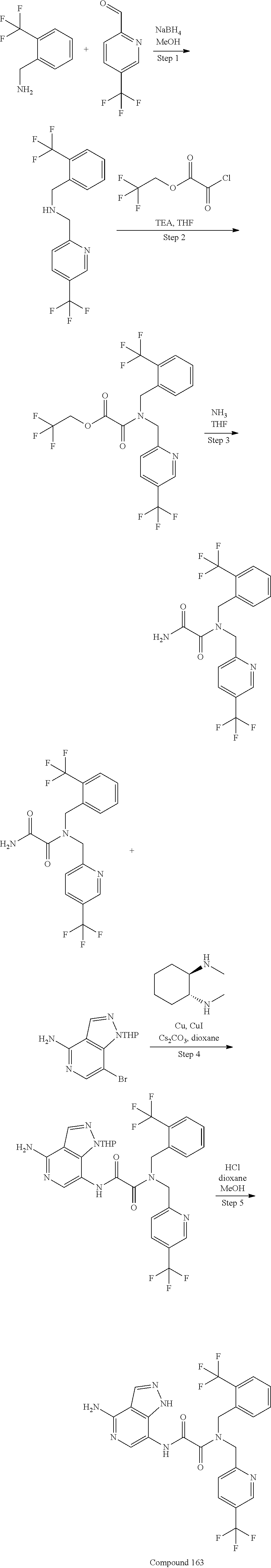

### Step 1:1-[2-(trifluoromethyl)phenyl]-N-[[5-(trifluoromethyl)-2-pyridyl]methyl]methanamine

A solution of 5-(trifluoromethyl)pyridine-2-carbaldehyde (0.7 g, 4.00 mmol) and [2-(trifluoromethyl)phenyl]methanamine (700.17 mg, 4.00 mmol, 560.59 μL) in MeOH (19.89 mL) was stirred at 20° C. for 12 hr. To this solution, Sodium Borohydride (166.35 mg, 4.40 mmol, 154.89 μL) was added and the resulting mixture was stirred for 5 hr. The solvent was removed in vacuo, the residue was taken up with water (40 mL) and extracted with DCM (3×20 mL). The combined organic layer was washed with brine (10 mL), dried over anhydrous sodium sulfate and evaporated to obtain 1-[2-(trifluoromethyl)phenyl]-N-[[5-(trifluoromethyl)-2-pyridyl]methyl]methanamine (1 g, 2.99 mmol, 74.84% yield) as a brown oil.

LCMS (ESI): [M−H]⁻ m/z: calcd 334.08; found 334.2; Rt=3.079 min.

### Step 2:2,2,2-trifluoroethyl 2-oxo-2-[[2-(trifluoromethyl)phenyl]methyl-[[5-(trifluoromethyl)-2-pyridyl]methyl]amino]acetate

To a solution of 1-[2-(trifluoromethyl)phenyl]-N-[5-(trifluoromethyl)-2-pyridyl]methyl]methanamine (1 g, 2.99 mmol) and TEA (454.09 mg, 4.49 mmol, 625.47 μL) in THF (30.11 mL) was added 2,2,2-trifluoroethyl 2-chloro-2-oxo-acetate (683.92 mg, 3.59 mmol) dropwise at 0° C. under argon. The reaction mixture was then stirred for 8 hr at r.t., then evaporated in vacuo to give 2,2,2-trifluoroethyl 2-oxo-2-[2-(trifluoromethyl)phenyl]methyl-[5-(trifluoromethyl)-2-pyridyl]methyl]amino]acetate (1,4 g, 2.87 mmol, 95.83% yield) as a light-yellow oil.

### Step 3: N′-[[2-(trifluoromethyl)phenyl]methyl]-N′-[[5-(trifluoromethyl)-2-pyridyl]methyl]oxamide

2,2,2-trifluoroethyl 2-oxo-2-[[2-(trifluoromethyl)phenyl]methyl-[[5-(trifluoromethyl)-2-pyridyl]methyl]amino]acetate (1.4 g, 2.87 mmol) was dissolved in THF (40 mL) and was blow ammonium (1.10 g, 60.74 mmol). The resulting solution was stirred at 0° C. for 5 hr. The resulting mixture was evaporated in vacuo and residue was triturated with THF (2×20 mL), filtered and combined organic was evaporated in vacuo to leave 1.1 g of crude product which was purification by column chromatography on silica gel using CHCl₃/CH₃CN gradient (10-100% CH₃CN) to afford N′-[2-(trifluoromethyl)phenyl]methyl]-N′-[5-(trifluoromethyl)-2-pyridyl]methyl]oxamide (0.6 g, 1.48 mmol, 51.63% yield) as a light-yellow oil.

LCMS (ESI): [M+H]⁺m/z: calcd 406.1; found 406.2; Rt=1.372 min.

### Step 4: N-(4-amino-1-tetrahydropyran-2-yl-pyrazolo[4,3-c]pyridin-7-yl)-N′-[[2-(trifluoromethyl)phenyl]methyl]-N′-[[5-(trifluoromethyl)-2-pyridyl]methyl]oxamide

7-bromo-1-tetrahydropyran-2-yl-pyrazolo[4,3-c]pyridin-4-amine (285.94 mg, 962.26 μmol), N′-[[2-(trifluoromethyl)phenyl]methyl]-N′-[5-(trifluoromethyl)-2-pyridyl]methyl]oxamide (0.3 g, 740.20 μmol), Cu(2.35 mg, 37.01 μmol), CuI (0.15 g, 787.61 μmol, 26.69 μL), cesium carbonate (361.76 mg, 1.11 mmol) and (1R,2R)—N1,N2-dimethylcyclohexane-1,2-diamine (0.15 g, 1.05 mmol) were mixed in dioxane (6.00 mL), purged with Ar for 15 minutes and then heated in the sealed tube at 105° C. for 48 hr. Final mixture was filtered and dioxane was evaporated in vacuo. The crude product 0.6 g was purified by RP-HPLC (column: XBridge C18 5 μm 130A; 50-50-90% 0-1.3-5.3 min H₂O/CH₃OH/0.1% NH₄OH, flow: 30 mL/min) to give N-(4-amino-1-tetrahydropyran-2-yl-pyrazolo[4,3-c]pyridin-7-yl)-N′-[[2-(trifluoromethyl)phenyl]methyl]-N′-[5-(trifluoromethyl)-2-pyridyl]methyl]oxamide (0.1467 g, 236.03 μmol, 31.89% yield) as a brown solid.

LCMS (ESI): [M+H]⁺m/z: calcd 622.22; found 622.0; Rt=3.516 min.

### Step 5: The synthesis of N-(4-amino-1H-pyrazolo[4,3-c]pyridin-7-yl)-N′-[[2-(trifluoromethyl)phenyl]methyl]-N′-[[5-(trifluoromethyl)-2-pyridyl]methyl]oxamide

Hydrogen chloride solution 4.0M in dioxane (4.00 g, 109.71 mmol, 5 mL) was added to a solution of N-(4-amino-1-tetrahydropyran-2-yl-pyrazolo[4,3-c]pyridin-7-yl)-N′-[2-(trifluoromethyl)phenyl]methyl]-N′-[5-(trifluoromethyl)-2-pyridyl]methyl]oxamide (0.1467 g, 236.03 μmol) in MeOH (8 mL). The reaction mixture was stirred at 20° C. for 24 hr, then evaporated was purified by RP-HPLC (column: XBridge BEH18 SMB100-BT 100×19 mm; 55-55-75% 0-1.5-5 min H₂O/CH₃OH/0.1% NH₄OH, flow: 30 mL/min) to give N-(4-amino-1H-pyrazolo[4,3-c]pyridin-7-yl)-N′-[[2-(trifluoromethyl)phenyl]methyl]-N′-[[5-(trifluoromethyl)-2-pyridyl]methyl]oxamide (0.0733 g, 136.39 μmol, 57.79% yield) as a light-yellow solid.

¹H NMR (600 MHz, DMSO-d₆) δ 4.60-4.88 (m, 2H), 5.07-5.25 (m, 2H), 6.59-7.08 (m, 2H), 7.46-7.59 (m, 2H), 7.61-7.64 (m, 1H), 7.69-7.76 (m, 2H), 8.06-8.25 (m, 3H), 8.83-8.95 (m, 1H), 10.57 (s, 1H), 12.80 (br s, 1H).

LCMS (ESI): [M+H]⁺m/z: calcd 538.16; found 538.2; Rt=2.688 min.

### Example 137. Synthesis of The Synthesis of rel-(S)-N1-(4-amino-1H-pyrazolo[4,3-c]pyridin-7-yl)-N2-(3-methylbutan-2-yl)-N2-((3-methylpyridin-2-yl)methyl)oxalamide (Compound 171) and rel-(R)—N¹-(4-amino-1H-pyrazolo[4,3-c]pyridin-7-yl)-N2-(3-methylbutan-2-yl)-N2-((3-methylpyridin-2-yl)methyl)oxalamide (Compound 106)

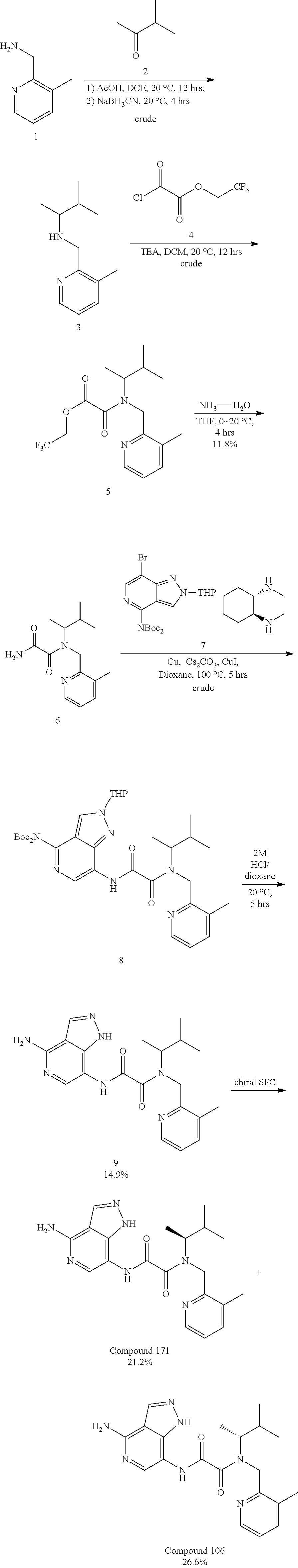

### Step 1: Synthesis of 3-methyl-N-((3-methylpyridin-2-yl)methyl) butan-2-amine

To a solution of (3-methyl-2-pyridyl)methanamine (500 mg, 4.09 mmol) in DCE (20 mL) was added acetic acid (125 mg, 2.08 mmol) and the mixture was stirred at 20° C. for 12 hours under N₂ atmosphere. NaBH₃CN(388 mg, 6.17 mmol) was added. The mixture was stirred at 20° C. for 4 hours. The reaction mixture was diluted with water 20 mL and extracted with EtOAc (50 mL*2). The combined organic layers were washed with brine (30 mL*3), dried over anhydrous Na₂SO₄, filtered and concentrated under reduced pressure to afford 3-methyl-N-[(3-methyl-2-pyridyl)methyl]butan-2-amine (900 mg, crude) as brown oil. LCMS (ESI) [M+H]⁺m/z: calcd 193.2, found 193.1

### Step 2: Synthesis of 2,2,2-trifluoroethyl 2-((3-methylbutan-2-yl)((3-methylpyridin-2-yl)methyl)amino)-2-oxoacetate

To a solution of 3-methyl-N-[(3-methyl-2-pyridyl)methyl]butan-2-amine (900 mg, 4.68 mmol) and 2,2,2-trifluoroethyl 2-chloro-2-oxo-acetate (1.46 g, 7.67 mmol) in DCM (20 mL) was added TEA (4.75 g, 47.0 mmol). The mixture was stirred at 20° C. for 12 hours. The resulting mixture was quenched by addition of water (10 mL) and extracted with DCM (100 mL*3). The combined organic layer was washed with brine (20 mL*3), dried over anhydrous Na₂SO₄, filtered and concentrated under reduced pressure to afford 2,2,2-trifluoroethyl 2-[1,2-dimethylpropyl-[(3-methyl-2-pyridyl)methyl]amino]-2-oxo-acetate (2.56 g, crude) as brown oil.

LCMS (ESI) [M+H]⁺m/z: calcd 347.2, found 347.1.

### Step 3: Synthesis of N1-(3-methylbutan-2-yl)-N1-((3-methylpyridin-2-yl)methyl)oxalamide

To a solution of 2,2,2-trifluoroethyl 2-[1,2-dimethylpropyl-[(3-methyl-2-pyridyl)methyl]amino]-2-oxo-acetate (2.56 g, 7.39 mmol) in THF (20 mL) was added Ammonium hydroxide, 28% solution (3 g, 85.6 mmol) at 0° C. . . . The mixture was stirred at 20° C. for 4 hours. The resulting mixture was quenched by addition of water (10 mL) and extracted with EtOAc (20 mL*3). The combined organic layer was washed with brine (20 mL*3), dried over anhydrous Na₂SO₄, filtered and concentrated under reduced pressure to afford a brown oil. The residue was purified by preparative HPLC (Instrument: Gilson GX-281 Liquid Handler, Gilson 322 Pump, Gilson 156 UV Detector; Column: 2 Phenomenex Gemini C18 75*40 mm*3 μm; Mobile phase A: H₂O with 0.05% NH₃—H₂O; Mobile phase B: ACN; Gradient: B from 20% to 50% in 7.8 min, hold 100% B for 3 min; Flow Rate: 25 mL/min; Column Temperature: 30° C.; Wavelength: 220 nm) to afford N′-(1,2-dimethylpropyl)-N′-[(3-methyl-2-pyridyl)methyl]oxamide (230 mg, 11.8% yield) as white solid. LCMS (ESI) [M+H]⁺m/z: calcd 264.2, found 264.1.

### Step 4: Synthesis of tert-butyl N-tert-butoxycarbonyl-N-[7-[2-[1,2-dimethylpropyl-[(3-methyl-2-pyridyl)methyl]amino]-2-oxo-acetyl]amino]-2-tetrahydropyran-2-yl-pyrazolo[4,3-c]pyridin-4-yl]carbamate

To a solution of N′-(1,2-dimethylpropyl)-N′-[(3-methyl-2-pyridyl)methyl]oxamide (230 mg, 0.873 mmol) in dioxane (20 mL) was added tert-butyl N-(7-bromo-2-tetrahydropyran-2-yl-pyrazolo[4,3-c]pyridin-4-yl)-N-tert-butoxycarbonyl-carbamate (436 mg, 0.877 mmol), Cu(61 mg, 0.960 mmol), CuI (170 mg, 0.893 mmol) and cesium carbonate (574 mg, 1.76 mmol). The mixture was stirred at 100° C. for 5 hours. The reaction mixture was diluted with EtOAc (100 mL) and extracted with NH3-H₂O(40 mL*3). The combined organic layers were washed with brine (30 mL*2), dried over anhydrous Na₂SO₄, filtered and concentrated under reduced pressure. The residue was purified by purified by flash chromatography (ISCO®; 20 g AgelaFlash® Silica Flash Column, petroleum ether/EtOAc with EtOAc from 0~50%, flow rate=45 mL/min) to afford tert-butyl N-tert-butoxycarbonyl-N-[7-[2-[1,2-dimethylpropyl-[(3-methyl-2-pyridyl)methyl]amino]-2-oxo-acetyl]amino]-2-tetrahydropyran-2-yl-pyrazolo[4,3-c]pyridin-4-yl]carbamate (810 mg, crude) as light yellow oil. LCMS (ESI) [M+H]⁺m/z: calcd 680.4, found 680.3.

### Step 5: Synthesis of N1-(4-amino-1H-pyrazolo[4,3-c]pyridin-7-yl)-N2-(3-methylbutan-2-yl)-N2-((3-methylpyridin-2-yl)methyl)oxalamide

To a solution of tert-butyl N-tert-butoxycarbonyl-N-[7-[2-[1,2-dimethylpropyl-[(3-methyl-2-pyridyl)methyl]amino]-2-oxo-acetyl]amino]-2-tetrahydropyran-2-yl-pyrazolo[4,3-c]pyridin-4-yl]carbamate (810 mg, 1.19 mmol) in HCl-dioxane (2M, 10 mL). The mixture was stirred at 20° C. for 5 hours. The reaction mixture was filtered and concentrated under reduced pressure. The residue was purified by preparative HPLC (Instrument: Gilson GX-281 Liquid Handler, Gilson 322 Pump, Gilson 156 UV Detector; Column: Welch Xtimate C18 100×40 mm×3 μm; Mobile phase A: H₂O with 0.05% FA (v %); Mobile phase B: ACN; Gradient: B from 5% to 35% in 8 min, hold 100% B for 2 min; Flow Rate: 30 mL/min; Column Temperature: 30° C.; Wavelength: 220 nm) to afford N-(4-amino-1H-pyrazolo[4,3-c]pyridin-7-yl)-N′-(1,2-dimethylpropyl)-N′-[(3-methyl-2-pyridyl)methyl]oxamide (70 mg, 177.01 μmol, 14.9% yield) as white solid. LCMS (ESI) [M+H]⁺m/z: calcd 396.2, found 396.0.

### Step 6: Synthesis of rel-(S)-N1-(4-amino-1H-pyrazolo[4,3-c]pyridin-7-yl)-N2-(3-methylbutan-2-yl)-N2-((3-methylpyridin-2-yl)methyl)oxalamide (Compound 171) and rel-(R)—N¹-(4-amino-1H-pyrazolo[4,3-c]pyridin-7-yl)-N2-(3-methylbutan-2-yl)-N2-((3-methylpyridin-2-yl)methyl)oxalamide (Compound 106)

N-(4-amino-1H-pyrazolo[4,3-c]pyridin-7-yl)-N′-(1,2-dimethylpropyl)-N′-[(3-methyl-2-pyridyl)methyl]oxamide (50 mg, 0.126 mmol) was separated by chiral SFC (Instrument: SFC-80Q; Column: DAICEL CHIRALPAK AD (250 mm*30 mm, 10 μm); Mobile phase: supercritical CO₂/EtOH (0.1% NH₃H₂O)=35/35; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm) to afford Compound 171 as white solid and Compound 106 as white solid.

Compound 171: (S)-N1-(4-amino-1H-pyrazolo[4,3-c]pyridin-7-yl)-N2-(3-methylbutan-2-yl)-N2-((3-methylpyridin-2-yl)methyl)oxalamide (10.6 mg, 21.2% yield, peak 1, retention time=1.920 min, white solid) ¹H NMR (400 MHz, DMSO-d₆) δ ppm 9.89 (br s, 1H), 8.34 (br d, J=4.27 Hz, 1H), 8.11-8.23 (m, 1H), 7.62-7.93 (m, 1H), 7.41-7.58 (m, 1H), 7.00-7.20 (m, 1H), 6.19-6.62 (m, 2H), 4.29-5.29 (m, 2H), 3.99-4.27 (m, 1H), 2.39 (s, 1H), 2.28 (s, 1H), 1.22-2.02 (m, 3H), 1.03 (br d, J=6.78 Hz, 2H), 0.71-1.20 (m, 8H); LCMS (ESI) [M+H]⁺m/z: calcd 396.2, found 396.2; HPLC: 97.4% @220 nm, 99.4% @254 nm.

Compound 106: (S)-N1-(4-amino-1H-pyrazolo[4,3-c]pyridin-7-yl)-N2-(3-methylbutan-2-yl)-N2-((3-methylpyridin-2-yl)methyl)oxalamide (13.3 mg, 26.6% yield, peak 2, retention time=2.067 min, white solid) ¹H NMR (400 MHz, DMSO-d₆) δ ppm 9.89 (br s, 1H), 8.34 (br d, J=4.27 Hz, 1H), 8.11-8.23 (m, 1H), 7.63-7.94 (m, 1H), 7.42-7.58 (m, 1H), 7.03-7.21 (m, 1H), 6.22-6.52 (m, 2H), 4.37-5.37 (m, 2H), 3.95-4.27 (m, 1H), 2.39 (br s, 1H), 1.89-2.07 (m, 1H), 1.17-1.56 (m, 3H), 0.78-1.14 (m, 9H); LCMS (ESI) [M+H]⁺m/z: calcd 396.2, found 396.2; HPLC: 97.9% @220 nm, 98.8% @254 nm.

### Example 138. Synthesis of N-(4-amino-1H-pyrazolo[4,3-c]pyridin-7-yl)-N′-[[3-methyl-5-(trifluoromethyl)-2-pyridyl]methyl]-N′-(pyrimidin-2-ylmethyl)oxamide (Compound 86)

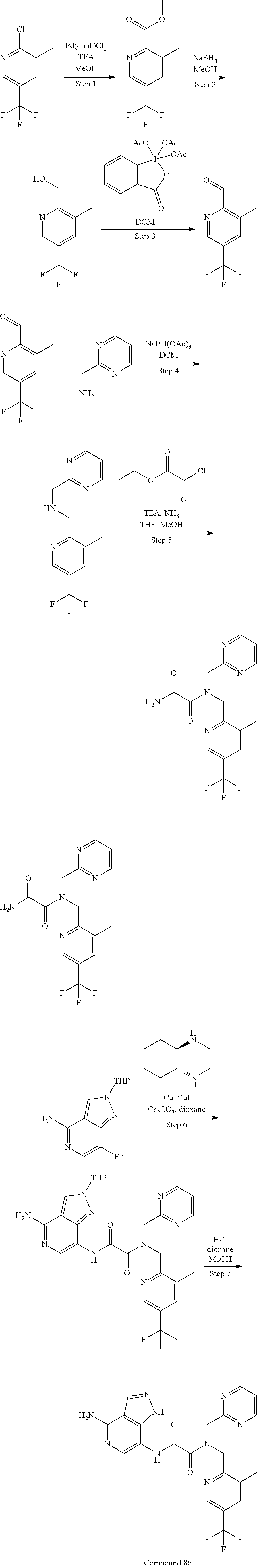

### Step 1: methyl 3-methyl-5-(trifluoromethyl)pyridine-2-carboxylate

A mixture of 2-chloro-3-methyl-5-(trifluoromethyl)pyridine (5 g, 25.57 mmol), Pd(dppf)Cl₂ (2.09 g, 2.56 mmol and triethyl amine (3.36 g, 33.24 mmol, 4.63 mL) in methanol (100 mL) was stirred under 10 atm. pressure of carbon monoxide in autoclave at 90° C. for 24 hr. The reaction mixture was cooled down and concentrated in vacuo. The residue was diluted with water (60 mL), extracted with MTBE (2×50 mL). The combined organic extracts were washed with water (20 mL), dried over anhydrous sodium sulfate and concentrated in vacuo to afford methyl 3-methyl-5-(trifluoromethyl)pyridine-2-carboxylate (5.1 g, 23.27 mmol, 91.02% yield) as a brown oil which was used directly in the next step.

LCMS (ESI): [M+H]⁺m/z: calcd 220.06; found 220.0; Rt=1.045 min.

### Step 2: [3-methyl-5-(trifluoromethyl)-2-pyridyl]methanol

Sodium Borohydride (1.76 g, 46.54 mmol, 1.64 mL) was added in small portions over 0.5 hr with stirring to a cooled to 0° C. solution of methyl 3-methyl-5-(trifluoromethyl)pyridine-2-carboxylate (5.1 g, 23.27 mmol) in methanol (75 mL). The resulting mixture was allowed to warm to 25° C. (cooling ice bath wasn't removed) and stirred for 12 hr. The reaction mixture was concentrated in vacuo, the residue was diluted with water (50 mL) and extracted with MTBE (2×50 mL). The combined organic extracts were dried over anhydrous sodium sulfate and concentrated in vacuo to afford [3-methyl-5-(trifluoromethyl)-2-pyridyl]methanol (3.6 g, 18.83 mmol, 80.93% yield) as a yellow gum.

LCMS (ESI): [M+H]⁺m/z: calcd 192.07; found 192.0; Rt=0.790 min.

### Step 3:3-methyl-5-(trifluoromethyl)pyridine-2-carbaldehyde

1,1-bis(acetyloxy)-3-oxo-3H-1-lambda-5,2-benziodaoxol-1-yl acetate (6.10 g, 14.39 mmol) was added in one portion to a cooled to 0° C. solution of [3-methyl-5-(trifluoromethyl)-2-pyridyl]methanol (2.5 g, 13.08 mmol) in dichloromethane (100 mL). The resulting mixture was allowed to warm to 25° C. over 1 hr period. The reaction mixture was carefully neutralized with 10% aqueous sodium carbonate solution. The resulting slurry was filtered, the filtercake was washed with dichloromethane (2×25 mL) and discarded. The combined filtrate was transferred to a separatory funnel, the dichloromethane layer was separated, dried over anhydrous sodium sulfate, and concentrated in vacuo. The residue was triturated with hexane (100 mL), stirred for 10 min, and then filtered. The filtercake was additionally washed with hexane (2×20 mL) and then discarded. The combined hexane filtrate was concentrated in vacuo to afford 3-methyl-5-(trifluoromethyl)pyridine-2-carbaldehyde (2.2 g, 11.63 mmol, 88.94% yield) as a yellow oil.

Step 4:1-[3-methyl-5-(trifluoromethyl)-2-pyridyl]-N-(pyrimidin-2-ylmethyl)methanamine

Pyrimidin-2-ylmethanamine (634.69 mg, 5.82 mmol) was added at 25° C. to a solution of 3-methyl-5-(trifluoromethyl)pyridine-2-carbaldehyde (1 g, 5.29 mmol) in dichloromethane (30 mL) followed by sodium triacetoxyborohydride (3.36 g, 15.86 mmol)(immediately!). The resulting mixture was stirred at 25° C. for 12 hr. The reaction mixture was carefully basified with 10% aqueous sodium hydroxide solution and then transferred to a separatory funnel. The organic layer was separated, dried over anhydrous sodium sulfate and concentrated in vacuo to afford 1-[3-methyl-5-(trifluoromethyl)-2-pyridyl]-N-(pyrimidin-2-ylmethyl)methanamine (1.6 g, crude) as a light-brown gum which was directly used in the next step.

LCMS (ESI): [M+H]⁺m/z: calcd 283.12; found 283.0; Rt=0.878 min.

### Step 5: N′-[[3-methyl-5-(trifluoromethyl)-2-pyridyl]methyl]-N′-(pyrimidin-2-ylmethyl)oxamide

Ethyl 2-chloro-2-oxo-acetate (503.06 mg, 3.68 mmol, 411.67 μL) was added slowly to a cooled to −10° C. mixture of 1-[3-methyl-5-(trifluoromethyl)-2-pyridyl]-N-(pyrimidin-2-ylmethyl)methanamine (800 mg, 2.83 mmol) and triethyl amine (1.15 g, 11.34 mmol, 1.58 mL) in THF (20 mL). The resulting mixture was allowed to warm to 25° C. and stirred for 1 hr. Then reaction mixture was diluted with methanol (40 mL) and gaseous ammonia (4.83 g, 283.42 mmol) was vigorously bubbled through it at 25° C. for 1 hr. The resulting mixture was stirred at 25° C. for 15 hr, and then concentrated in vacuo. The residue was diluted with water (30 mL) and extracted with MTBE/ethyl acetate mixture (1/1, 2×25 mL). The combined organic extracts were dried over anhydrous sodium sulfate and concentrated in vacuo to afford N′-[[3-methyl-5-(trifluoromethyl)-2-pyridyl]methyl]-N′-(pyrimidin-2-ylmethyl)oxamide (760 mg, 2.15 mmol, 75.90% yield) as a yellow gum which was used directly in the next step.

LCMS (ESI): [M+H]⁺m/z: calcd 354.12; found 354.0; Rt=1.007 min.

### Step 6: N-(4-amino-2-tetrahydropyran-2-yl-pyrazolo[4,3-c]pyridin-7-yl)-N′-[[3-methyl-5-(trifluoromethyl)-2-pyridyl]methyl]-N′-(pyrimidin-2-ylmethyl)oxamide

A mixture of N′-[3-methyl-5-(trifluoromethyl)-2-pyridyl]methyl]-N′-(pyrimidin-2-ylmethyl)oxamide (300 mg, 849.14 μmol), 7-bromo-2-tetrahydropyran-2-yl-pyrazolo[4,3-c]pyridin-4-amine (378.48 mg, 1.27 mmol), copper (5 mg, 78.68 μmol), Copper (I) iodide (150 mg, 787.61 μmol, 26.69 μL), cesium carbonate (442.67 mg, 1.36 mmol) and rac-(1R,2R)—N1,N2-dimethylcyclohexane-1,2-diamine (150 mg, 1.05 mmol) in 1,4-dioxane (7 mL) was stirred in a sealed vial under argon at 105° C. for 18 hr. The resulting mixture was cooled down and filtered. The filtercake was washed successively with THF (2×5 mL) and dichloromethane (3×5 mL). The combined filtrate was concentrated in vacuo to afford N-(4-amino-2-tetrahydropyran-2-yl-pyrazolo[4,3-c]pyridin-7-yl)-N′-[[3-methyl-5-(trifluoromethyl)-2-pyridyl]methyl]-N′-(pyrimidin-2-ylmethyl)oxamide (900 mg, crude) as a brown gum which was used directly in the next step.

LCMS (ESI): [M+H]⁺m/z: calcd 570.22; found 570.2; Rt=2.150 min.

### Step 7: The synthesis of N-(4-amino-1H-pyrazolo[4,3-c]pyridin-7-yl)-N′-[[3-methyl-5-(trifluoromethyl)-2-pyridyl]methyl]-N′-(pyrimidin-2-ylmethyl)oxamide

Hydrogen chloride solution 4.0M in dioxane (5.25 g, 20.02 mmol, 5 mL, 13.9% purity) was added to a stirred solution of crude from previous step N-(4-amino-2-tetrahydropyran-2-yl-pyrazolo[4,3-c]pyridin-7-yl)-N′-[[3-methyl-5-(trifluoromethyl)-2-pyridyl]methyl]-N′-(pyrimidin-2-ylmethyl)oxamide (0.9 g, 1.58 mmol) in methanol (5 mL) at 25 C. The resulting solution was stirred at 25° C. for 12 hr, then concentrated to dryness in vacuo and the residue was submitted to reverse phase HPLC (column: Chromatorex 18 SNB100-5T 100×9 mm 5 μm; mobile phase: 0-0-30% 0-1.3-5.3 min H₂O/ACN/0.1% FA; flow: 30 mL/min (loading pump 4 mL/min acetonitrile)) to afford N-(4-amino-1H-pyrazolo[4,3-c]pyridin-7-yl)-N′-[3-methyl-5-(trifluoromethyl)-2-pyridyl]methyl]-N′-(pyrimidin-2-ylmethyl)oxamide (48 mg, 90.32 μmol, 5.72% yield, HCOOH) as a light-brown solid.

¹H NMR (600 MHz, DMSO-d₆) δ 2.29-2.44 (m, 3H), 4.88-4.93 (m, 2H), 5.23-5.33 (m, 2H), 7.09-7.36 (m, 2H), 7.37-7.44 (m, 1H), 7.56-7.70 (m, 1H), 7.92-8.04 (m, 1H), 8.26 (s, 1H), 8.71-8.72 (m, 1H), 8.73-8.75 (m, 1H), 8.76-8.81 (m, 1H), 10.45 (s, 1H), 12.84-13.42 (m, 1H).

LCMS (ESI): [M+H]⁺m/z: calcd 486.17; found 486.2; Rt=2.343 min.

### Example 139. Synthesis of N-(4-amino-1H-pyrazolo[4,3-c]pyridin-7-yl)-N′-[(2-fluorophenyl)methyl]-N′-isobutyl-oxamide (Compound 94)

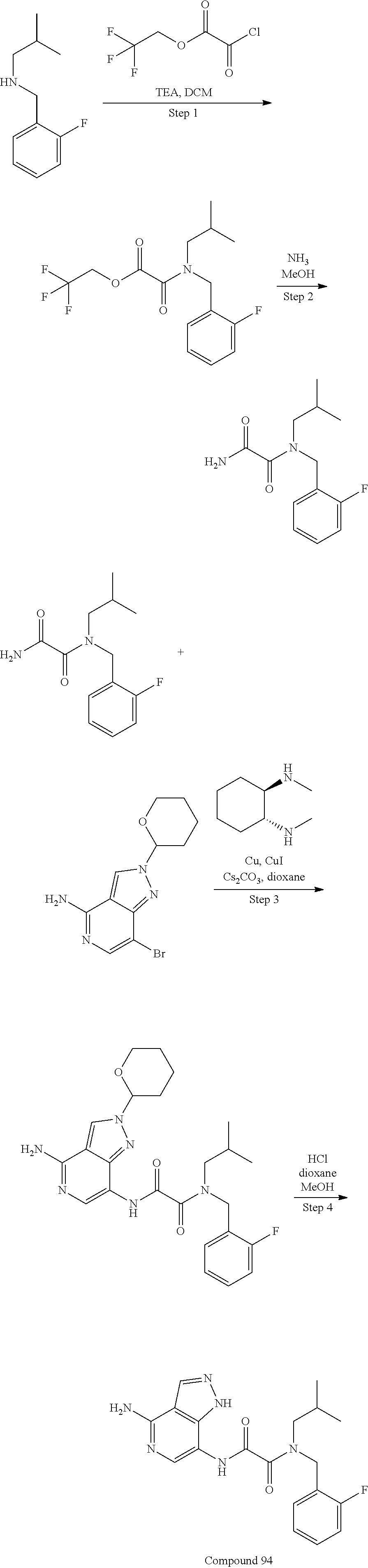

### Step 1:2,2,2-trifluoroethyl 2-[(2-fluorophenyl)methyl-isobutyl-amino]-2-oxo-acetate

N-[(2-fluorophenyl)methyl]-2-methyl-propan-1-amine (508.1 mg, 2.80 mmol) and Triethylamine (312.04 mg, 3.08 mmol, 429.80 μL) were dissolved in DCM (12 mL) and the resulting solution was cooled to −5° C. in an ice/methanol bath. 2,2,2-trifluoroethyl 2-chloro-2-oxo-acetate (560.75 mg, 2.94 mmol) was added dropwise and the resulting mixture was stirred overnight. Water (15 mL) was added to the reaction mixture and an organic layer was separated. The aqueous layer was extracted with DCM (15 mL) and combined organic layers were dried over anhydrous sodium sulfate, filtered, and concentrated in vacuo to obtain 2,2,2-trifluoroethyl 2-[(2-fluorophenyl)methyl-isobutyl-amino]-2-oxo-acetate (857 mg, 2.56 mmol, 91.18% yield) as a light-yellow oil.

LCMS (ESI): [M+H]⁺m/z: calcd 336.12; found 336.2; Rt=1.380 min.

### Step 2: N′-[(2-fluorophenyl)methyl]-N′-isobutyl-oxamide

2,2,2-trifluoroethyl 2-[(2-fluorophenyl)methyl-isobutyl-amino]-2-oxo-acetate (786 mg, 2.34 mmol) was dissolved in MeOH (10 mL) and NH3/MeOH (15 mL) was added thereto. The resulting mixture was stirred overnight. The reaction mixture was concentrated in vacuo to obtain N′-[(2-fluorophenyl)methyl]-N′-isobutyl-oxamide (540 mg, 2.14 mmol, 91.31% yield) as a light-yellow gum.

LCMS (ESI): [M+H]⁺m/z: calcd 253.14; found 253.0; Rt=1.027 min.

### Step 3: N-(4-amino-2-tetrahydropyran-2-yl-pyrazolo[4,3-c]pyridin-7-yl)-N′-[(2-fluorophenyl)methyl]-N′-isobutyl-oxamide

All components were partitioned between two 8 ml vials. N′-[(2-fluorophenyl)methyl]-N′-isobutyl-oxamide (300 mg, 1.19 mmol), 7-bromo-2-tetrahydropyran-2-yl-pyrazolo[4,3-c]pyridin-4-amine (388.69 mg, 1.31 mmol), Copper (3.78 mg, 59.46 μmol), Copper (I) iodide (113.24 mg, 594.57 μmol, 20.15 μL), rac-(1R,2R)—N1,N2-dimethylcyclohexane-1,2-diamine (126.86 mg, 891.85 μmol) and Cesium carbonate (774.89 mg, 2.38 mmol) were mixed in Dioxane (8 mL). The reaction mixture was splurged with argon for 5 min. The vials were sealed and heated at 100° C. for 40 hr. The reaction mixture was cooled and filtered. The filtercake was rinsed with MeOH (10 mL) and the filtrate was concentrated in vacuo to obtain N-(4-amino-2-tetrahydropyran-2-yl-pyrazolo[4,3-c]pyridin-7-yl)-N′-[(2-fluorophenyl)methyl]-N′-isobutyl-oxamide (1.02 g, crude) as a greenish solid which was used in the next step without purification.

LCMS (ESI): [M+H]⁺m/z: calcd 469.24; found 469.2; Rt=1.259 min.

### Step 4: The synthesis of N-(4-amino-1H-pyrazolo[4,3-c]pyridin-7-yl)-N′-[(2-fluorophenyl)methyl]-N′-isobutyl-oxamide

N-(4-amino-2-tetrahydropyran-2-yl-pyrazolo[4,3-c]pyridin-7-yl)-N′-[(2-fluorophenyl)methyl]-N′-isobutyl-oxamide (1.02 g, 2.18 mmol) was dissolved in MeOH (6 mL) and HCl/dioxane (6 mL) was added thereto. The resulting mixture was stirred for 3 hr and then concentrated in vacuo. The residue was purified by HPLC (0-2-10 min, 38-45-70 H₂O/MeOH/0.1NH4OH, flow 30 mL/min ((loading pump 4 mL MeCN), target mass 385, column: Chromatorex C18 SMB100-5T 100×19 mm, 5 microM) to obtain N-(4-amino-1H-pyrazolo[4,3-c]pyridin-7-yl)-N′-[(2-fluorophenyl)methyl]-N′-isobutyl-oxamide (125.1 mg, 325.44 μmol, 14.95% yield) as a light-yellow solid.

¹H NMR (600 MHz, DMSO-d₆) δ 0.76-0.89 (m, 6H), 1.87-2.10 (m, 1H), 3.07-3.12 (m, 1H), 3.36-3.41 (m, 1H), 4.34-4.96 (m, 2H), 6.62-6.88 (m, 2H), 7.15-7.25 (m, 2H), 7.31-7.50 (m, 2H), 7.61-7.71 (m, 1H), 8.10-8.21 (m, 1H), 9.87-10.90 (m, 1H), 12.55-13.48 (m, 1H).

LCMS (ESI): [M+H]⁺m/z: calcd 385.2; found 385.2; Rt=0.790 min.

### Example 140. Synthesis of N-(4-amino-1H-pyrazolo[4,3-c]pyridin-7-yl)-N′-isobutyl-N′-(1-phenylethyl)oxamide (Compound 172), (R)—N¹-(4-amino-1H-pyrazolo[4,3-c]pyridin-7-yl)-N2-isobutyl-N2-(1-phenylethyl)oxalamide (Compound 129) and(S)-N1-(4-amino-1H-pyrazolo[4,3-c]pyridin-7-yl)-N2-isobutyl-N2-(1-phenylethyl)oxalamide (Compound 165)

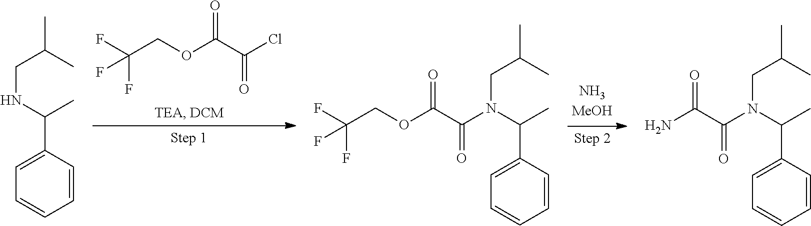

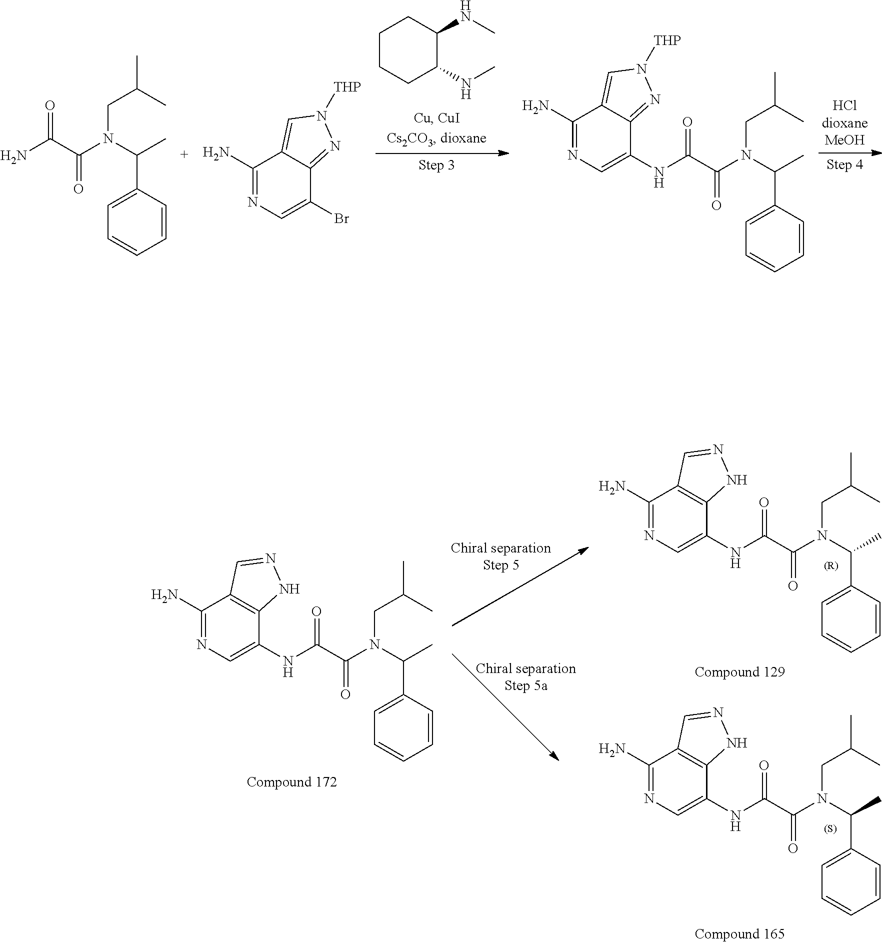

### Step 1:2,2,2-trifluoroethyl 2-[isobutyl(1-phenylethyl)amino]-2-oxo-acetate

2-methyl-N-(1-phenylethyl)propan-1-amine (507.7 mg, 2.38 mmol, HCl) was mixed in DCM (10 mL) and Triethylamine (600.88 mg, 5.94 mmol, 827.66 μL) was added. The resulting mixture was stirred for 30 min and the mixture was cooled to −5° C. in an ice/methanol bath. 2,2,2-trifluoroethyl 2-chloro-2-oxo-acetate (475.12 mg, 2.49 mmol) was added dropwise and the resulting mixture was stirred overnight. Water (15 mL) was added to the reaction mixture and an organic layer was separated. The aqueous layer was extracted with DCM (15 mL) and combined organic layers were dried over anhydrous sodium sulfate, filtered, and concentrated in vacuo to obtain 2,2,2-trifluoroethyl 2-[isobutyl(1-phenylethyl)amino]-2-oxo-acetate (717 mg, 2.16 mmol, 91.11% yield) as a yellow oil.

LCMS (ESI): [M+H]⁺m/z: calcd 332.15; found 332.2; Rt=1.408 min.

### Step 2: N′-isobutyl-N′-(1-phenylethyl)oxamide

2,2,2-trifluoroethyl 2-[isobutyl(1-phenylethyl)amino]-2-oxo-acetate (717 mg, 2.16 mmol) was dissolved in MeOH (10 mL) and NH3/MeOH (15 mL) was added thereto. The resulting solution was stirred overnight. The reaction mixture was concentrated in vacuo to obtain N′-isobutyl-N′-(1-phenylethyl)oxamide (570 mg, crude) as a light-yellow gum.

LCMS (ESI): [M−H]⁻ m/z: calcd 247.14; found 247.1; Rt=1.275 min.

### Step 3: N-(4-amino-2-tetrahydropyran-2-yl-pyrazolo[4,3-c]pyridin-7-yl)-N′-isobutyl-N′-(1-phenylethyl)oxamide

All components were partitioned between two 8 ml vials. N′-isobutyl-N′-(1-phenylethyl)oxamide (300 mg, 1.21 mmol), 7-bromo-2-tetrahydropyran-2-yl-pyrazolo[4,3-c]pyridin-4-amine (394.89 mg, 1.33 mmol), Copper (3.84 mg, 60.41 μmol), Copper (I) iodide (115.04 mg, 604.06 μmol, 20.47 μL), rac-(1R,2R)—N1,N2-dimethylcyclohexane-1,2-diamine (128.88 mg, 906.09 μmol) and Cesium carbonate (787.26 mg, 2.42 mmol) were mixed in Dioxane (8 mL). The resulting mixture was splurged with argon for 5 min. The vials were sealed and heated at 100° C. over weekend. The reaction mixture was cooled and filtered. The filtercake was washed with MeOH (10 mL) and the filtrate was concentrated in vacuo to obtain N-(4-amino-2-tetrahydropyran-2-yl-pyrazolo[4,3-c]pyridin-7-yl)-N′-isobutyl-N′-(1-phenylethyl)oxamide (1.4 g, crude) as a greenish solid which was used in the next step without purification.

LCMS (ESI): [M+H]⁺m/z: calcd 465.26; found 465.2; Rt=1.212 min.

### Step 4: The synthesis of N-(4-amino-1H-pyrazolo[4,3-c]pyridin-7-yl)-N′-isobutyl-N′-(1-phenylethyl)oxamide

N-(4-amino-2-tetrahydropyran-2-yl-pyrazolo[4,3-c]pyridin-7-yl)-N′-isobutyl-N′-(1-phenylethyl)oxamide (1.4 g, 3.01 mmol) was dissolved in MeOH (6 mL) and HCl/Dioxane (6 mL) was added thereto. The resulting mixture was stirred for 3 hr and then concentrated in vacuo. The residue was purified by HPLC (0-2-10 min, 33-40-55 H₂O/MeOH/0.1NH4OH, flow 30 mL/min ((loading pump 4 mL MeOH), target mass 380, column: XBridge BEH C18 100×19 mm, 5 microM) to obtain N-(4-amino-1H-pyrazolo[4,3-c]pyridin-7-yl)-N′-isobutyl-N′-(1-phenylethyl)oxamide (129.3 mg, 339.87 μmol, 11.28% yield) as a light-yellow solid.

¹H NMR (600 MHz, DMSO-d₆) § 0.30-0.76 (m, 6H), 1.60-1.65 (m, 3H), 1.65-2.01 (m, 1H), 2.73-3.19 (m, 2H), 4.99-5.53 (m, 1H), 6.63-6.82 (m, 2H), 7.21-7.30 (m, 1H), 7.30-7.38 (m, 2H), 7.38-7.50 (m, 2H), 7.50-7.77 (m, 1H), 8.12-8.22 (m, 1H), 10.47 (s, 1H), 12.73 (s, 1H).

LCMS (ESI): [M+H]⁺m/z: calcd 381.23; found 381.2; Rt=1.036 min.

### Step 5: The synthesis of (R)—N¹-(4-amino-1H-pyrazolo[4,3-c]pyridin-7-yl)-N2-isobutyl-N2-(1-phenylethyl)oxalamide (Compound 129)

N-(4-amino-1H-pyrazolo[4,3-c]pyridin-7-yl)-N′-isobutyl-N′-(1-phenylethyl)oxamide (57.5 mg, 151.14 μmol) was chirally separated (Column: CHIRALCEL OJ-H (250×20 mm, 5 mkm)-I, Mobile phase: Hexane: IPA: MeOH, 60:20:20. Flow Rate: 12 mL/min; Column Temperature: 24° C.; Wavelength: 205 nm. RetTime=12.52 min to obtain (R)—N¹-(4-amino-1H-pyrazolo[4,3-c]pyridin-7-yl)-N2-isobutyl-N2-(1-phenylethyl)oxalamide (23.52 mg, 61.82 μmol, 81.81% yield) as a light-yellow solid.

### Preparative:

Column: CHIRALCEL OJ-H (250×20 mm, 5 mkm)-I, Mobile phase: Hexane: IPA: MeOH, 60:20:20. Flow Rate: 12 mL/min; Column Temperature: 24° C.; Wavelength: 205 nm. RetTime=12.52 min;

### Analytical:

Column: Chiralcel OJ-H (250×4.6 mm, 5 mkm)-1, Mobile Phase: Hexane (0.1% EDA): IPA: MeOH, 50:25:25, Flow Rate: 0.6 mL/min. RetTime=10.476 min.

¹H NMR (600 MHz, DMSO-d₆) δ 0.28-0.75 (m, 6H), 1.28-1.72 (m, 4H), 2.72-3.21 (m, 2H), 4.98-5.53 (m, 1H), 6.59-6.83 (m, 2H), 7.19-7.49 (m, 5H), 7.51-7.76 (m, 1H), 8.10-8.21 (m, 1H), 9.36-10.70 (m, 1H), 12.59-13.39 (m, 1H).

LCMS (ESI): [M+H]⁺m/z: calcd 381.23; found 381.2; Rt=2.317 min

### Step 5a: The synthesis of(S)-N1-(4-amino-1H-pyrazolo[4,3-c]pyridin-7-yl)-N2-isobutyl-N2-(1-phenylethyl)oxalamide (Compound 165)

N-(4-amino-1H-pyrazolo[4,3-c]pyridin-7-yl)-N′-isobutyl-N′-(1-phenylethyl)oxamide (57.5 mg, 151.14 μmol) was chirally separated (Column: CHIRALCEL OJ-H (250×20 mm, 5 mkm)-I, Mobile phase: Hexane: IPA: MeOH, 60:20:20. Flow Rate: 12 mL/min; Column Temperature: 24° C.; Wavelength: 205 nm. RetTime=19.38 min to obtain(S)-N1-(4-amino-1H-pyrazolo[4,3-c]pyridin-7-yl)-N2-isobutyl-N2-(1-phenylethyl)oxalamide (23.4 mg, 61.51 μmol, 81.39% yield) as a light-yellow solid.

### Preparative:

Column: CHIRALCEL OJ-H (250×20 mm, 5 mkm)-I, Mobile phase: Hexane: IPA: MeOH, 60:20:20. Flow Rate: 12 mL/min; Column Temperature: 24° C.; Wavelength: 205 nm. RetTime=19.38 min;

### Analytical:

Column: Chiralcel OJ-H (250×4.6 mm, 5 mkm)-1, Mobile Phase: Hexane (0.1% EDA): IPA: MeOH, 50:25:25, Flow Rate: 0.6 mL/min. RetTime=17.543 min.

¹H NMR (600 MHz, DMSO-d₆) δ 0.26-1.05 (m, 6H), 1.55-1.68 (m, 3H), 2.71-3.20 (m, 2H), 4.98-5.53 (m, 1H), 6.60-6.82 (m, 2H), 6.85-7.50 (m, 5H), 7.52-7.78 (m, 1H), 8.12-8.22 (m, 1H), 9.36-10.55 (m, 1H), 12.59-13.36 (m, 1H).

LCMS (ESI): [M+H]⁺m/z: calcd 381.23; found 381.4; Rt=1.761 min.

### Example 141. Synthesis of N-(4-amino-1H-pyrazolo[4,3-c]pyridin-7-yl)-N′-methyl-N′-[[4-(pentafluoro-sulfanyl)phenyl]methyl]oxamide (Compound 100)

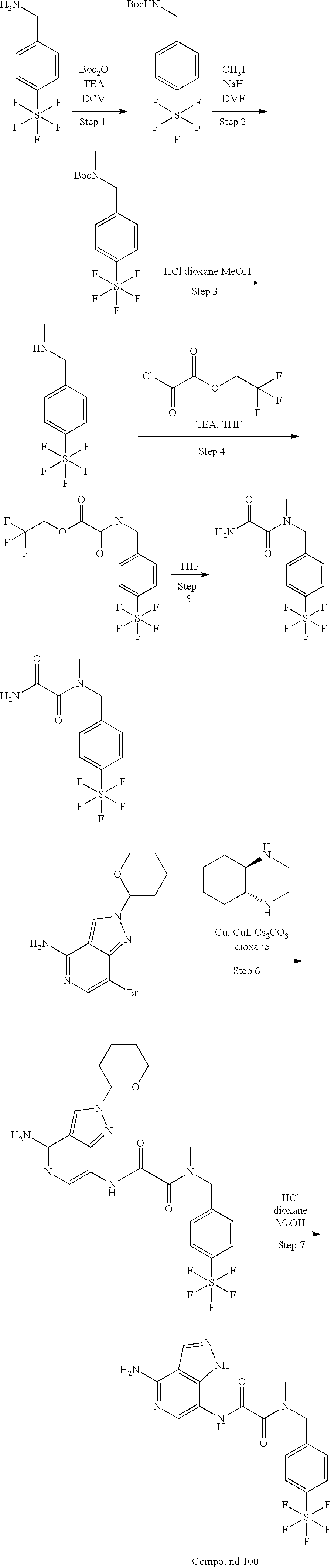

### Step 1: tert-butyl N-[[4-(pentafluoro-sulfanyl)phenyl]methyl]carbamate

To a solution of [4-(pentafluoro-sulfanyl)phenyl]methanamine (2 g, 7.42 mmol, HCl) and Triethylamine (1.50 g, 14.83 mmol, 2.07 mL) in DCM (50 mL), Di-tert-butyl dicarbonate (1.54 g, 7.05 mmol, 1.62 mL) was added. The resulting mixture was stirred at 25° C. for 12 hr, washed with water (2×20 mL) and NaHSO₄ solution (2×20 mL), dried over anhydrous sodium sulfate and evaporated in vacuo to obtain tert-butyl N-[4-(pentafluoro-sulfanyl)phenyl]methyl]carbamate (2.4 g, 7.20 mmol, 97.08% yield) as a light-yellow solid.

LCMS (ESI): [M-t-Bu]-m/z: calcd 278.0; found 278.0; Rt=1.544 min.

### Step 2: tert-butyl N-methyl-N-[[4-(pentafluoro-sulfanyl)phenyl]methyl]carbamate

To a stirred at 0° C. solution of tert-butyl N-[[4-(pentafluoro-sulfanyl)phenyl]methyl]carbamate (1.2 g, 3.60 mmol) in DMF (15 mL), Sodium Hydride (in oil dispersion) 60% dispersion in mineral oil (287.99 mg, 7.20 mmol, 60% purity) was added. After 30 min, Methyl iodide (1.02 g, 7.20 mmol, 448.25 μL) was added. The resulting mixture was stirred at 25° C. for 12 hr and poured into water (80 mL) and extracted with MTBE (3×15 mL), dried over anhydrous sodium sulfate and evaporated in vacuo to obtain tert-butyl N-methyl-N-[4-(pentafluoro-sulfanyl)phenyl]methyl]carbamate (1.1 g, 3.17 mmol, 87.96% yield) as a light-yellow oil.

LCMS (ESI): [M-t-Bu]-m/z: calcd 292.0; found 292.0; Rt=1.593 min.

### Step 3: N-methyl-1-[4-(pentafluoro-sulfanyl)phenyl]methanamine

A solution of tert-butyl N-methyl-N-[4-(pentafluoro-sulfanyl)phenyl]methyl]carbamate (1.1 g, 3.17 mmol) in MeOH (15 mL) and Hydrogen chloride solution 4.0M in dioxane (5 g, 137.13 mmol, 6.25 mL) was stirred at 25° C. for 4 hr. The solvent was evaporated to give N-methyl-1-[4-(pentafluoro-sulfanyl)phenyl]methanamine (890 mg, 3.14 mmol, 99.06% yield, HCl) as a light-yellow solid.

LCMS (ESI): [M+H]⁺m/z: calcd 248.06; found 248.0; Rt=0.938 min.

### Step 4:2,2,2-trifluoroethyl 2-[methyl-[[4-(pentafluoro-sulfanyl)phenyl]methyl]amino]-2-oxo-acetate

To a solution of N-methyl-1-[4-(pentafluoro-sulfanyl)phenyl]methanamine (890 mg, 3.14 mmol, HCl) and Triethylamine (1.59 g, 15.69 mmol, 2.19 mL) in THF (50 mL), 2,2,2-trifluoroethyl 2-chloro-2-oxo-acetate (1.49 g, 7.84 mmol) was added portionwise at 0° C. The resulting mixture was stirred for 3 hr. LCMS showed full conversion of SM. The reaction mixture was directly used for the next step.

LCMS (ESI): [M+H]⁺m/z: calcd 402.04; found 402.0; Rt=1.440 min.

### Step 5: N′-methyl-N′-[[4-(pentafluoro-sulfanyl)phenyl]methyl]oxamide

Through a solution of 2,2,2-trifluoroethyl 2-[methyl-[[4-(pentafluoro-sulfanyl)phenyl]methyl]amino]-2-oxo-acetate (1.25 g, 3.12 mmol) in THF (50 mL), ammonia was bubbled during 10 min at 0° C. The formed precipitate was filtered off, washed with THF (30 mL) and the solvent was evaporated in vacuo to give crude product (0.9 g), which was purified by gradient chromatography (CHCl₃-ACN) to afford N′-methyl-N′-[4-(pentafluoro-sulfanyl)phenyl]methyl]oxamide (0.25 g, 785.51 μmol, 25.22% yield) as a white solid.

LCMS (ESI): [M+H]⁺m/z: calcd 319.06; found 319.0; Rt=1.036 min.

### Step 6: N-(4-amino-2-tetrahydropyran-2-yl-pyrazolo[4,3-c]pyridin-7-yl)-N′-methyl-N′-[[4-(pentafluoro-sulfanyl)phenyl]methyl]oxamide

To a mixture of N′-methyl-N′-[4-(pentafluoro-sulfanyl)phenyl]methyl]oxamide (250 mg, 785.51 μmol), 7-bromo-2-tetrahydropyran-2-yl-pyrazolo[4,3-c]pyridin-4-amine (350.12 mg, 1.18 mmol), Copper (9.98 mg, 157.10 μmol), Copper (I) iodide (149.60 mg, 785.51 μmol, 26.62 μL), Cesium carbonate (511.87 mg, 1.57 mmol) and rac-(1R,2R)—N1,N2-dimethylcyclohexane-1,2-diamine (167.60 mg, 1.18 mmol), Dioxane (5 mL) was added. The resulting mixture was evacuated, refiled with Argon three time, heated at 100° C. for 18 hr and cooled. The inorganic precipitate was filtered off and washed with DCM (30 mL) to give N-(4-amino-2-tetrahydropyran-2-yl-pyrazolo[4,3-c]pyridin-7-yl)-N′-methyl-N′-[[4-(pentafluoro-sulfanyl)phenyl]methyl]oxamide (0.4 g, crude). This substance was used for the next step without further purification.

LCMS (ESI): [M+H]⁺m/z: calcd 535.16; found 535.1; Rt=1.256 min.

### Step 7: The synthesis of N-(4-amino-1H-pyrazolo[4,3-c]pyridin-7-yl)-N′-methyl-N′-[[4-(pentafluoro-sulfanyl)phenyl]methyl]oxamide

To a solution of N-(4-amino-2-tetrahydropyran-2-yl-pyrazolo[4,3-c]pyridin-7-yl)-N′-methyl-N′-[4-(pentafluoro-sulfanyl)phenyl]methyl]oxamide (0.4 g, 748.36 μmol) in MeOH (10 mL), Hydrogen chloride solution 4.0M in dioxane (4 g, 109.71 mmol, 5.00 mL) was added. The resulting mixture was stirred at 25° C. for 5 hr and evaporated in vacuo. The residue was purified by HPLC (Device (Mobile Phase, Column): SYSTEM 20-20-65% 0-1-5 min H₂O/ACN/0.2% FA, flow: 50 ml/min (loading pump 4 mL/min acetonitrile) target mass 450; 436 column: Agilent 5 PrepC18 100×30 mm, 5 μm) to obtain N-(4-amino-1H-pyrazolo[4,3-c]pyridin-7-yl)-N′-methyl-N′-[4-(pentafluoro-sulfanyl)phenyl]methyl]oxamide (85 mg, 171.23 μmol, 22.88% yield, HCO₂H) as a light-yellow solid.

LCMS (ESI): [M+H]⁺m/z: calcd 451.11; found 451.0; Rt=2.714 min.

### Example 142. Synthesis of N-(4-Amino-1H-pyrazolo[4,3-c]pyridin-7-yl)-N′-cyclopropyl-N′-[[5-(trifluoromethyl)-2-pyridyl]methyl]oxamide (Compound 159)

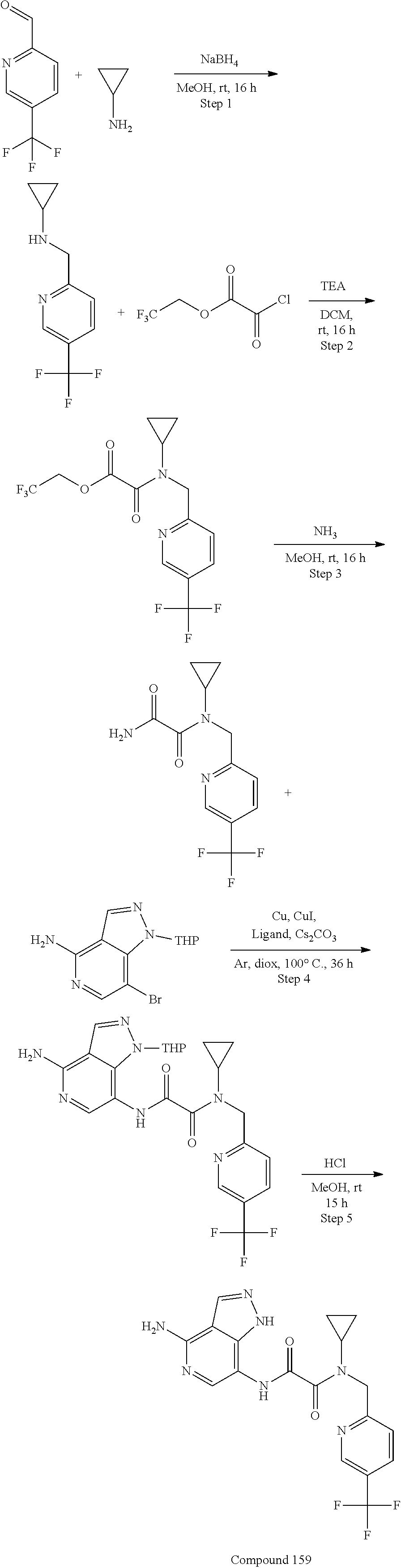

### Step 1: The Synthesis of N-[5-(Trifluoromethyl)-2-pyridyl]methyl]cyclopropanamine

Cyclopropanamine (586.89 mg, 10.28 mmol, 712.25 μL) were added to the solution of 5-(trifluoromethyl)pyridine-2-carbaldehyde (0.9 g, 5.14 mmol) in MeOH (10 mL). Resulting mixture was stirred at 60° C. for 1 hour before Sodium Borohydride (388.90 mg, 10.28 mmol, 362.10 μL) was added portions thereto. After that, stirring was continued for 16 hr at rt. Then, solvent was removed under reduced pressure and residue was partitioned between 10% aq. K₂CO₃ solution and DCM. Organic layer was separated, dried over solid Na₂SO₄ and concentrated under reduced pressure, leaving N-[5-(trifluoromethyl)-2-pyridyl]methyl]cyclopropanamine (0.5 g, crude).

LCMS (ESI): [M+3H]⁺m/z: calcd 219.2; found 219.2; Rt=0.438 min.

### Step 2: The Synthesis of 2-[Cyclopropyl-[5-(trifluoromethyl)-2-pyridyl]methyl]amino]-2-oxo-acetate

2,2,2-Trifluoroethyl 2-chloro-2-oxo-acetate (276.24 mg, 1.45 mmol) was added dropwise to a solution of N-[5-(trifluoromethyl)-2-pyridyl]methyl]cyclopropanamine (285 mg, 1.32 mmol) and triethylamine (160.07 mg, 1.58 mmol, 220.48 L) in DCM (10 mL) at 0° C. After addition was complete, cooling bath was removed and resulting mixture was allowed to warm up to 24° C. and stirred for 16 hr. Then, it was washed with water, dried over Na₂SO₄ and concentrated under reduced pressure, affording 2,2,2-trifluoroethyl 2-[cyclopropyl-[[5-(trifluoromethyl)-2-pyridyl]methyl]amino]-2-oxo-acetate (405 mg, crude) which was used in the next step without further purification.

LCMS (ESI): [M+H]⁺m/z: calcd 371.0; found 371.0; Rt=1.454 min.

### Step 3: The Synthesis of N′-Cyclopropyl-N′-[[5-(trifluoromethyl)-2-pyridyl]methyl]oxamide

A solution of 2,2,2-trifluoroethyl 2-[cyclopropyl-[[5-(trifluoromethyl)-2-pyridyl]methyl]amino]-2-oxo-acetate (405 mg, 1.09 mmol) in Methanol/NH3 (5N)(5 mL) was stirred at 25° C. for 16 hr. The solvent was evaporated to obtain N′-cyclopropyl-N′-[[5-(trifluoromethyl)-2-pyridyl]methyl]oxamide (330 mg, crude).

LCMS (ESI): [M+H]⁺m/z: calcd 288.0; found 288.0; Rt=1.028 min.

### Step 4: The Synthesis of N-(4-Amino-2-tetrahydropyran-2-yl-pyrazolo[4,3-c]pyridin-7-yl)-N′-cyclopropyl-N′-[5-(trifluoromethyl)-2-pyridyl]methyl]oxamide

N′-Cyclopropyl-N′-[[5-(trifluoromethyl)-2-pyridyl]methyl]oxamide (152 mg, 529.18 μmol), 7-bromo-2-tetrahydropyran-2-yl-pyrazolo[4,3-c]pyridin-4-amine (165.11 mg, 555.64 μmol), Copper (I) iodide (20.16 mg, 105.84 μmol, 3.59 μL), Cesium carbonate (344.83 mg, 1.06 mmol) and (1R,2R)—N1,N2-dimethylcyclohexane-1,2-diamine (90.32 mg, 635.02 μmol) were mixed in dioxane (5 mL) under argon, and then stirred overnight at 100° C. for 36 hr in vial. The reaction mixture was filtered and the filtrate was concentrated in vacuo to give N-(4-amino-2-tetrahydropyran-2-yl-pyrazolo[4,3-c]pyridin-7-yl)-N′-cyclopropyl-N′-[5-(trifluoromethyl)-2-pyridyl]methyl]oxamide (380 mg, crude).

LCMS (ESI): [M+H]⁺m/z: calcd 504.2; found 504.2; Rt=1.088 min.

### Step 5: The Synthesis of N-(4-Amino-1H-pyrazolo[4,3-c]pyridin-7-yl)-N′-cyclopropyl-N′-[[5-(trifluoromethyl)-2-pyridyl]methyl]oxamide (Compound 159)

To a solution of N-(4-amino-2-tetrahydropyran-2-yl-pyrazolo[4,3-c]pyridin-7-yl)-N′-cyclopropyl-N′-[5-(trifluoromethyl)-2-pyridyl]methyl]oxamide (380 mg, 754.75 μmol) in MeOH (4 mL) was added Hydrogen chloride solution 4.0M in dioxane (800.00 mg, 21.94 mmol, 1 mL) at 25° C. The resulting mixture was left to stirred for 15 hr. The resulting mixture was evaporated to dryness and then submitted to reverse phase HPLC (0-2-9 min 23-30-55% H₂O/MeOH/0.1NH4OH; flow 30 mL/min ((loading pump 4 mL MEOH); column: XBridge BEH C18 100*19 mm, 5 microM) to afford N-(4-amino-1H-pyrazolo[4,3-c]pyridin-7-yl)-N′-cyclopropyl-N′-[5-(trifluoromethyl)-2-pyridyl]methyl]oxamide (17.9 mg, 42.68 μmol, 5.66% yield)

¹H NMR (500 MHz, cd3od) δ 0.82-0.89 (m, 2H), 0.89-0.99 (m, 2H), 3.09-3.18 (m, 1H), 7.62 (d, 1H), 7.69 (s, 1H), 8.13 (dd, 1H), 8.18-8.25 (m, 1H), 8.86 (s, 1H).

LCMS (ESI): [M+H]⁺m/z: calcd 420.2; found 420.2; Rt=0.751 min.

### Example 143. Synthesis of N-(4-amino-1H-pyrazolo[4,3-c]pyridin-7-yl)-N′-ethyl-N′-[[4-(pentafluoro-sulfanyl)phenyl]methyl]oxamide (Compound 150)

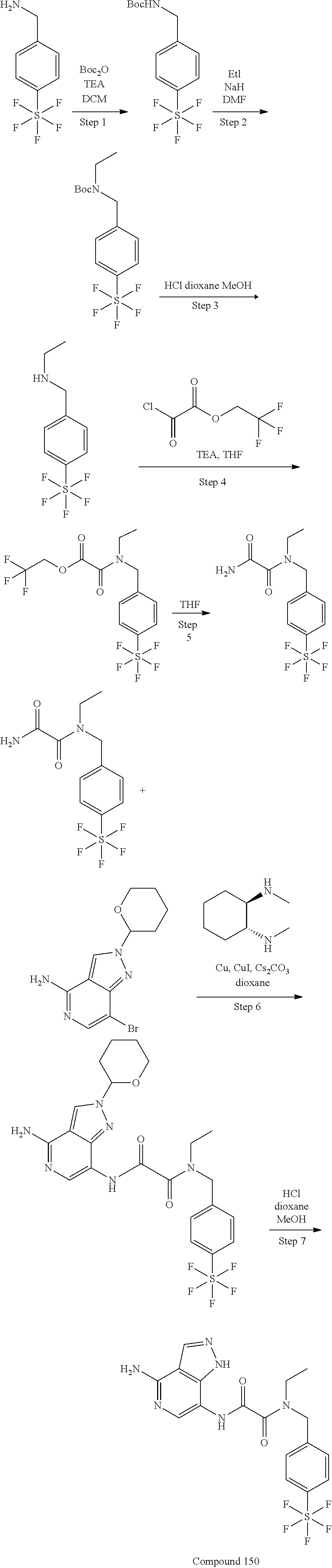

### Step 1: tert-butyl N-[4-(pentafluoro-sulfanyl)phenyl]methyl]carbamate

To a solution of [4-(pentafluoro-sulfanyl)phenyl]methanamine (2 g, 7.42 mmol, HCl) and Triethylamine (1.50 g, 14.83 mmol, 2.07 mL) in DCM (50 mL), Di-tert-butyl dicarbonate (1.54 g, 7.05 mmol, 1.62 mL) was added. The resulting mixture was stirred at 25° C. for 12 hr, washed with water (2×20 mL) and NaHSO₄ solution (2×20 mL), dried over anhydrous sodium sulfate and evaporated in vacuo to obtain tert-butyl N-[[4-(pentafluoro-sulfanyl)phenyl]methyl]carbamate (2.4 g, 7.20 mmol, 97.08% yield) as a light-yellow solid.

LCMS (ESI): [M-t-Bu]; m/z: calcd 278.0; found 278.0; Rt=1.544 min.

### Step 2: tert-butyl N-ethyl-N-[[4-(pentafluoro-sulfanyl)phenyl]methyl]carbamate

To a stirred at 0° C. solution of tert-butyl N-[[4-(pentafluoro-sulfanyl)phenyl]methyl]carbamate (1.2 g, 3.60 mmol) in DMF (15 mL), Sodium Hydride (in oil dispersion) 60% dispersion in mineral oil (288.01 mg, 7.20 mmol, 60% purity) was added. After 30 min, Ethyl iodide (1.12 g, 7.20 mmol, 578.87 μL) was added. The resulting mixture was stirred at 25° C. for 12 hr and poured into water (80 mL) and extracted with MTBE (3×15 mL), dried over anhydrous sodium sulfate and evaporated in vacuo to obtain tert-butyl N-ethyl-N-[[4-(pentafluoro-sulfanyl)phenyl]methyl]carbamate (1.2 g, 3.32 mmol, 92.24% yield) as a light-yellow oil.

LCMS (ESI): [M-t-Bu]-m/z: calcd 306.0; found 306.0; Rt=1.648 min.

### Step 3: N-[[4-(pentafluoro-sulfanyl)phenyl]methyl]ethanamine

A solution of tert-butyl N-ethyl-N-[[4-(pentafluoro-sulfanyl)phenyl]methyl]carbamate (1.2 g, 3.32 mmol) in MeOH (25 mL) and Hydrogen chloride solution 4.0M in dioxane (8 g, 219.41 mmol, 10.00 mL) was stirred at 25° C. for 3 hr. The solvent was evaporated to give N-[[4-(pentafluoro-sulfanyl)phenyl]methyl]ethanamine (980 mg, 3.29 mmol, 99.13% yield, HCl) as a light-yellow solid.

LCMS (ESI): [M+H]⁺m/z: calcd 262.07; found 262.0; Rt=0.969 min.

### Step 4:2,2,2-trifluoroethyl 2-[ethyl-[4-(pentafluoro-sulfanyl)phenyl]methyl]amino]-2-oxo-acetate

To a solution of N-[4-(pentafluoro-sulfanyl)phenyl]methyl]ethanamine (980 mg, 3.29 mmol, HCl) and Triethylamine (1.67 g, 16.46 mmol, 2.29 mL) in THF (50 mL), 2,2,2-trifluoroethyl 2-chloro-2-oxo-acetate (1.57 g, 8.23 mmol) was added portionwise at 0° C. The resulting mixture was stirred for 4 hr. LCMS showed full conversion of SM. The reaction mixture was directly used for the next step.

LCMS (ESI): [M+H]⁺m/z: calcd 416.06; found 416.0; Rt=1.478 min.

### Step 5: N′-ethyl-N′-[4-(pentafluoro-sulfanyl)phenyl]methyl]oxamide

Through a solution of 2,2,2-trifluoroethyl 2-[ethyl-[[4-(pentafluoro-sulfanyl)phenyl]methyl]amino]-2-oxo-acetate (1.35 g, 3.25 mmol) in THF (50 mL), ammonia was bubbled during 10 min at 0° C. The formed precipitate was filtered off, washed with THF (30 mL) and the solvent was evaporated in vacuo to give crude product (0.9 g), which was purified by gradient chromatography (CHCl₃-ACN) to afford N′-ethyl-N′-[4-(pentafluoro-sulfanyl)phenyl]methyl]oxamide (430 mg, 1.29 mmol, 39.81% yield) as a light-yellow solid.

LCMS (ESI): [M+H]⁺m/z: calcd 333.07; found 333.0; Rt=1.254 min.

### Step 6: N-(4-amino-2-tetrahydropyran-2-yl-pyrazolo[4,3-c]pyridin-7-yl)-N′-ethyl-N′-[[4-(pentafluoro-sulfanyl)phenyl]methyl]oxamide

To a mixture of N′-ethyl-N′-[4-(pentafluoro-sulfanyl)phenyl]methyl]oxamide (230 mg, 692.17 μmol), 7-bromo-2-tetrahydropyran-2-yl-pyrazolo[4,3-c]pyridin-4-amine (308.52 mg, 1.04 mmol), Copper (8.80 mg, 138.43 μmol), Copper (I) iodide (131.82 mg, 692.17 μmol, 23.46 μL), Cesium carbonate (451.04 mg, 1.38 mmol) and rac-(1R,2R)—N1,N2-dimethylcyclohexane-1,2-diamine (147.68 mg, 1.04 mmol), Dioxane (6 mL) was added. The resulting mixture was evacuated, refiled with Argon three time, heated at 100° C. for 18 hr and cooled. The inorganic precipitate was filtered off and washed with DCM (30 mL) to give N-(4-amino-2-tetrahydropyran-2-yl-pyrazolo[4,3-c]pyridin-7-yl)-N′-ethyl-N′-[[4-(pentafluoro-sulfanyl)phenyl]methyl]oxamide (0.35 g, crude).

This substance was used for the next step without further purification.

LCMS (ESI): [M+H]⁺m/z: calcd 549.17; found 549.0; Rt=1.298 min.

### Step 7: The synthesis of N-(4-amino-1H-pyrazolo[4,3-c]pyridin-7-yl)-N′-ethyl-N′-[[4-(pentafluoro-sulfanyl)phenyl]methyl]oxamide

To a solution of N-(4-amino-2-tetrahydropyran-2-yl-pyrazolo[4,3-c]pyridin-7-yl)-N′-ethyl-N′-[[4-(pentafluoro-sulfanyl)phenyl]methyl]oxamide (0.35 g, 638.07 μmol) in MeOH (10 mL), Hydrogen chloride solution 4.0M in dioxane (4 g, 109.71 mmol, 5.00 mL) was added. The resulting mixture was stirred at 25° C. for 5 hr and evaporated in vacuo. The residue was purified by HPLC (Device (Mobile Phase, Column): SYSTEM Oct. 10, 2040% 0-1-5 min H₂O/ACN/0.2% FA, flow: 50 mL/min (loading pump 4 mL/min acetonitrile) target mass 464 column: Agilent 5 PrepC18 100×30 mm 5 μm) to obtain N-(4-amino-1H-pyrazolo[4,3-c]pyridin-7-yl)-N′-ethyl-N′-[4-(pentafluoro-sulfanyl)phenyl]methyl]oxamide (109 mg, 213.54 μmol, 33.47% yield, HCO₂H) as a light-yellow solid.

LCMS (ESI): [M+H]⁺m/z: calcd 465.13; found 465.2; Rt=2.057 min.

### Example 144. Synthesis of N-(4-amino-1H-pyrazolo[4,3-c]pyridin-7-yl)-N′-methyl-N′-[1-[4-(1,1,2,2,2-pentafluoroethyl)phenyl]ethyl]oxamide (Compound 123), N-(4-amino-1H-pyrazolo[4,3-c]pyridin-7-yl)-N′-methyl-N′—[(1R)-1-[4-(1,1,2,2,2-pentafluoroethyl)phenyl]ethyl]oxamide (Compound 121) and N-(4-amino-1H-pyrazolo[4,3-c]pyridin-7-yl)-N′-methyl-N′—[(1S)-1-[4-(1,1,2,2,2-pentafluoroethyl)phenyl]ethyl]oxamide (Compound 131)

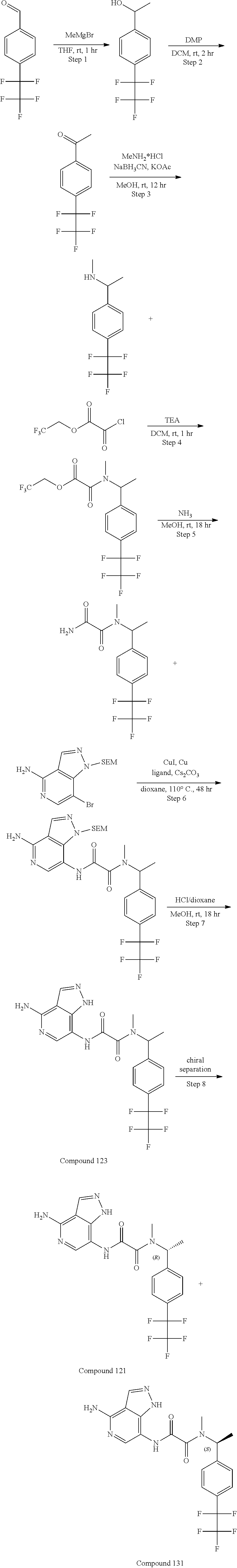

### Step 1: The Synthesis of 1-[4-(1,1,2,2,2-pentafluoroethyl)phenyl]ethanol

Methylmagnesium bromide (798.05 mg, 6.69 mmol, 2.1 mL) was added to the solution of 4-(1,1,2,2,2-pentafluoroethyl)benzaldehyde (1 g, 4.46 mmol) in THF (25 mL) maintaining the internal temperature below 25° C. The resulting reaction mixture was allowed to stir at room temperature for 1 hr, then quenched with saturated NH₄Cl solution and extracted with EtOAc (2×25 mL). The combined organic layer was dried over Na₂SO₄, filtered, and concentrated under reduced pressure to obtain product 1-[4-(1,1,2,2,2-pentafluoroethyl)phenyl]ethanol (1 g, 4.16 mmol, 93.32% yield) as a light-yellow oil, which was used in the next step reaction without any further purification.

### Step 2: The Synthesis of 1-[4-(1,1,2,2,2-pentafluoroethyl)phenyl]ethanone

To a solution of 1-[4-(1,1,2,2,2-pentafluoroethyl)phenyl]ethanol (1 g, 4.16 mmol) in DCM (25 mL) was added Dess-Martin Periodinane (1.94 g, 4.58 mmol) in one portion. The resulting mixture was stirred 2 h at rt. The reaction mixture was poured into a solution containing Na₂S₂O₃ and Na₂CO₃ (2:1 by weight), stirred for 30 min, DCM was dried and evaporated. 1-[4-(1,1,2,2,2-pentafluoroethyl)phenyl]ethanone (0.9 g, 3.78 mmol, 90.76% yield) was obtained as a white solid.

### Step 3: The Synthesis of N-methyl-1-[4-(1,1,2,2,2-pentafluoroethyl)phenyl]ethanamine

To a solution of 1-[4-(1,1,2,2,2-pentafluoroethyl)phenyl]ethanone (0.9 g, 3.78 mmol) in MeOH (50 mL) was added Sodium cyanoborohydride (356.23 mg, 5.67 mmol) and N-methylamine (2.55 g, 37.79 mmol, 2.84 mL, HCl). The mixture was stirred at 20° C. for 12 h. The reaction was quenched by adding water (100 mL) and was extracted with EtOAc (100 mL×2). The combined organic layers were washed with brine (2×50 mL), dried over anhydrous Na₂SO₄ and concentrated under reduced pressure to give N-methyl-1-[4-(1,1,2,2,2-pentafluoroethyl)phenyl]ethanamine (0.65 g, 2.57 mmol, 67.93% yield) as a white solid.

LCMS (ESI): [M+H]⁺m/z: calcd 254.1; found 254.2; RT=2.25 min.

### Step 4: The Synthesis of 2,2,2-trifluoroethyl 2-[methyl-[1-[4-(1,1,2,2,2-pentafluoroethyl)phenyl]ethyl]amino]-2-oxo-acetate

To a solution of N-methyl-1-[4-(1,1,2,2,2-pentafluoroethyl)phenyl]ethanamine (0.65 g, 2.57 mmol) and TEA (259.76 mg, 2.57 mmol, 357.79 μL) in DCM (25 mL) was added 2,2,2-trifluoroethyl 2-chloro-2-oxo-acetate (489.03 mg, 2.57 mmol) at rt. After stirring at rt for 1 hr the resulting mixture was washed with water, dried and evaporated to dryness to give 2,2,2-trifluoroethyl 2-[methyl-[1-[4-(1,1,2,2,2-pentafluoroethyl)phenyl]ethyl]amino]-2-oxo-acetate (0.9 g, 2.21 mmol, 86.09% yield) as a yellow gum and was used in the next step without further purification.

LCMS (ESI): [M+H]⁺m/z: calcd 408.1; found 408.0; RT=3.995 min.

### Step 5: The Synthesis of N′-methyl-N′-[1-[4-(1,1,2,2,2-pentafluoroethyl)phenyl]ethyl]oxamide

Ammonia (37.64 mg, 2.21 mmol) was bubbled through a solution of 2,2,2-trifluoroethyl 2-[methyl-[1-[4-(1, 1,2,2,2-pentafluoroethyl)phenyl]ethyl]amino]-2-oxo-acetate (0.9 g, 2.21 mmol) in MeOH (25 mL) at rt (while bubbling an exotherm was observed). After stirring for 18 hr, the reaction mixture was evaporated to dryness to give a residue which was purified by CC (Interchim; 40 g SiO₂, chloroform/acetonitrile with acetonitrile from 0~30%, flow rate=40 mL/min, Rv=11-13CV) to give N′-methyl-N′-[1-[4-(1,1,2,2,2-pentafluoroethyl)phenyl]ethyl]oxamide (0.5 g, 1.54 mmol, 69.78% yield) as a pale-yellow gum.

LCMS (ESI): [M+Na]⁺m/z: calcd 347.1; found 347.0; RT=1.324 min.

### Step 6: The Synthesis of N-[4-amino-1-(2-trimethylsilylethoxymethyl)pyrazolo[4,3-c]pyridin-7-yl]-N′-methyl-N′-[1-[4-(1,1,2,2,2-pentafluoroethyl)phenyl]ethyl]oxamide

N′-methyl-N′-[1-[4-(1,1,2,2,2-pentafluoroethyl)phenyl]ethyl]oxamide (0.2 g, 616.82 μmol), 7-bromo-1-(2-trimethylsilylethoxymethyl)pyrazolo[4,3-c]pyridin-4-amine (211.75 mg, 616.82 μmol), Copper (I) iodide (58.74 mg, 308.41 μmol, 10.45 μL), Cesium carbonate (401.94 mg, 1.23 mmol) and (1R,2R)—N1,N2-dimethylcyclohexane-1,2-diamine (87.74 mg, 616.82 μmol) were mixed in dioxane (5.00 mL) under argon, and then stirred for 48 h at 100° C. for 48 hr in vial. The reaction mixture was filtered and concentrated in vacuo. The residue was used in the next step without further purification.

LCMS (ESI): [M+H]⁺m/z: calcd 587.2; found 587.2; RT=3.705 min.

### Step 7: The Synthesis of N-(4-amino-1H-pyrazolo[4,3-c]pyridin-7-yl)-N′-methyl-N′-[1-[4-(1,1,2,2,2-pentafluoroethyl)phenyl]ethyl]oxamide (Compound 123)

To a solution of N-[4-amino-1-(2-trimethylsilylethoxymethyl)pyrazolo[4,3-c]pyridin-7-yl]-N′-methyl-N′-[1-[4-(1,1,2,2,2-pentafluoroethyl)phenyl]ethyl]oxamide (0.5 g, 136.37 μmol, 16% purity) in MeOH (3.07 mL) was added Hydrogen chloride solution 4.0M in dioxane (731.43 mg, 20.06 mmol, 914.29 μL) at 21° C. The resulting mixture was left to stir for 18 hr. The resulting mixture was evaporated to dryness and subjected to HPLC (25-50% 2-7 min; 30 ml/min water-acetonitrile+NH3 (loading pump 4 ml/min acetonitrile); target mass 457; column XBRIDGE19*100 mm (L)). N-(4-amino-1H-pyrazolo[4,3-c]pyridin-7-yl)-N′-methyl-N′-[1-[4-(1,1,2,2,2-pentafluoroethyl)phenyl]ethyl]oxamide (44.9 mg, 98.39 μmol, 72.14% yield) was obtained as a brown solid.

LCMS (ESI): [M+H]⁺m/z: calcd 457.2; found 457.2; RT=2.508 min.

### Step 8: The Synthesis of N-(4-amino-1H-pyrazolo[4,3-c]pyridin-7-yl)-N′-methyl-N′—[(1R)-1-[4-(1,1,2,2,2-pentafluoroethyl)phenyl]ethyl]oxamide (Compound 121) and N-(4-amino-1H-pyrazolo[4,3-c]pyridin-7-yl)-N′-methyl-N′—[(1S)-1-[4-(1,1,2,2,2-pentafluoroethyl)phenyl]ethyl]oxamide (Compound 131)

The enantiomers were separated by chiral HPLC:
Injection Volume: 900mkl
Column: CHIRALCEL OJ-H (250×20 mm, 5 mkm)
Mobile Phase: Hexane (0.1% DEA): IPA: MeOH, 80:10:10
Flow Rate: 16 ml/min
RT of R¹ Compound 121=16.7 min

LCMS (ESI): [M+H]⁺m/z: calcd 457.2; found 457.2; RT=2.15 min.

¹H NMR (600 MHz, DMSO-d₆) δ 13.24 (d, J=34.9 Hz, 1H), 12.74 (d, J=31.0 Hz, 1H), 9.70 (d, J=12.1 Hz, 1H), 8.16 (d, J=22.1 Hz, 1H), 7.95-7.23 (m, 3H), 7.08-6.46 (m, 2H), 5.91-5.22 (m, 2H), 2.90 (s, 1H), 2.61 (s, 1H), 2.30 (s, 1H), 1.62 (dd, J=41.3, 7.0 Hz, 2H), 1.48-0.75 (m, 3H).
RT of R² Compound 131=27.8 min (contaminated)
R² fr was purified additionally
Injection Volume: 900mkl
Column: CHIRALCEL OJ-H (250×20 mm, 5 mkm)
Mobile Phase: Hexane (0.1% DEA): IPA: MeOH, 80:10:10
Flow Rate: 16 ml/min
RT of Compound 131=28.52 min.

LCMS (ESI): [M+H]⁺m/z: calcd 457.2; found 457.2; RT=2.14 min.

¹H NMR (600 MHz, DMSO-d₆) δ 13.24 (d, J=35.3 Hz, 1H), 12.73 (d, J=30.4 Hz, 1H), 10.57 (s, 1H), 9.70 (d, J=12.6 Hz, 1H), 8.16 (d, J=20.3 Hz, 1H), 7.90-7.26 (m, 3H), 7.03-6.29 (m, 2H), 5.70 (dd, J=121.3, 7.1 Hz, 1H), 2.90 (s, 1H), 2.61 (s, 1H), 2.30 (s, 1H), 1.62 (dd, J=41.3, 7.0 Hz, 1H), 1.47-0.68 (m, 2H).

The absolute stereochemistry of the two compounds was independently confirmed

### Example 145. Synthesis of N-(4-amino-1H-pyrazolo[4,3-c]pyridin-7-yl)-N′-methyl-N′-[1-[5-(trifluoromethyl)-2-pyridyl]propyl]oxamide (Compound 138)

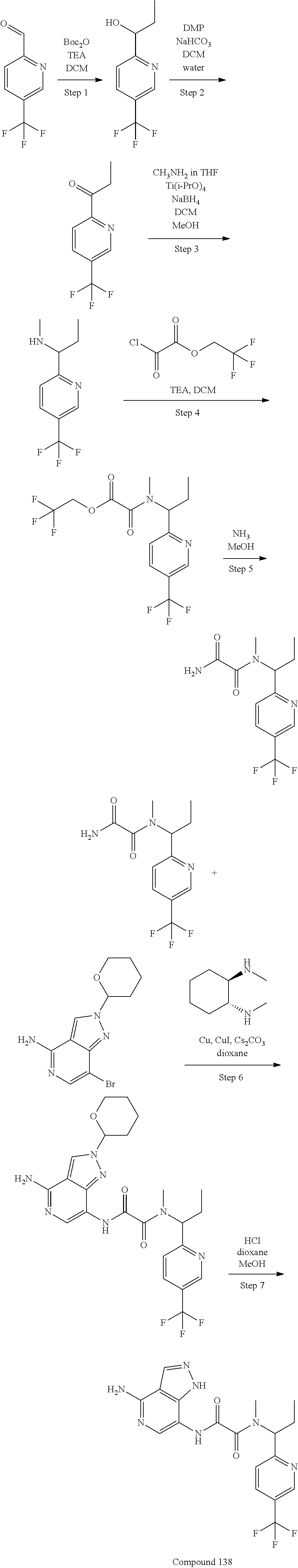

### Step 1:1-[5-(trifluoromethyl)-2-pyridyl]propan-1-ol

Ethylmagnesium chloride (2.7M in THF)(2.23 g, 6.28 mmol, 2.32 mL, 25% purity) was added dropwise to the stirred solution of 5-(trifluoromethyl)pyridine-2-carbaldehyde (1 g, 5.71 mmol) in Tetrahydrofuran (30 mL) at −60° C. under argon. After addition was complete, resulting reaction mixture was stirred for 30 min at the same temperature. Then, cooling bath was removed, and mixture was gradually warmed up to −20° C. At this point reaction was quenched with saturated aq. NH4Cl solution (30 mL). MTBE (30 mL) was added to the mixture and organic layer was separated, dried over anhydrous sodium sulfate and concentrated under reduced pressure, affording 1-[5-(trifluoromethyl)-2-pyridyl]propan-1-ol (1.06 g, 5.17 mmol, 90.47% yield) as a yellow oil.

LCMS (ESI): [M+H]⁺m/z: calcd 206.08; found 206.0; Rt=0.727 min.

### Step 2:1-[5-(trifluoromethyl)-2-pyridyl]propan-1-one

Dess-Martin Periodinane (2.85 g, 6.72 mmol) was added portionwise to the stirred solution of 1-[5-(trifluoromethyl)-2-pyridyl]propan-1-ol (1.06 g, 5.17 mmol) in Dichloromethane (30 mL). The resulting reaction mixture was stirred at 25° C. for 3 hr. Then, it was diluted with Water (40 mL) and Sodium bicarbonate (2.17 g, 25.83 mmol, 1.01 mL) was added portionwise. After addition was complete, resulting biphasic mixture was stirred for 16 hours at ambient temperature. Then, precipitate was filtered off and filtrate was transferred to separatory funnel. Organic layer was separated, and aqueous layer was extracted with DCM (30 mL). Combined organic layers was dried over anhydrous sodium sulfate and concentrated under reduced pressure, affording 1-[5-(trifluoromethyl)-2-pyridyl]propan-1-one (760 mg, 3.74 mmol, 72.41% yield) as a yellow oil.

### Step 3: N-methyl-1-[5-(trifluoromethyl)-2-pyridyl]propan-1-amine

To the stirred solution of 1-[5-(trifluoromethyl)-2-pyridyl]propan-1-one (760 mg, 3.74 mmol) in Dichloromethane (30 mL) was added in one portion Methylamine, 10% w/w in THF (3.49 g, 11.22 mmol, 4.10 mL, 10% purity) followed by Titanium (IV) isopropoxide (1.38 g, 4.86 mmol, 1.45 mL). The resulting mixture was stirred at 25° C. for 20 hr. Then, Sodium Borohydride (141.52 mg, 3.74 mmol, 131.77 μL) was added thereto followed by Methanol (4 mL) and stirring was continued for 30 min. After that, saturated aqueous K₂CO₃ solution (5 ml) was added. Resulting thick white precipitate was filtered off and filtrate was concentrated under reduced pressure, affording N-methyl-1-[5-(trifluoromethyl)-2-pyridyl]propan-1-amine (0.8 g, 3.67 mmol, 98.00% yield) as a yellow oil.

LCMS (ESI): [M+H]⁺m/z: calcd 219.11; found 219.2; Rt=0.507 min.

### Step 4:2,2,2-trifluoroethyl 2-[methyl-[1-[5-(trifluoromethyl)-2-pyridyl]propyl]amino]-2-oxo-acetate

2,2,2-trifluoroethyl 2-chloro-2-oxo-acetate (977.76 mg, 5.13 mmol) was added dropwise to the ice-cooled solution of N-methyl-1-[5-(trifluoromethyl)-2-pyridyl]propan-1-amine (0.8 g, 3.67 mmol) and Triethylamine (741.94 mg, 7.33 mmol, 1.02 mL) in Dichloromethane (25 mL). After addition was complete, cooling bath was removed and resulting mixture was allowed to warm up to 25° C. and stirred for 2 hr. Then, 10% aq. NaHCO₃ solution (15 mL) was added and stirring was continued for 10 minutes. Then, organic layer was separated, dried over anhydrous sodium sulfate and concentrated under reduced pressure, affording 2,2,2-trifluoroethyl 2-[methyl-[1-[5-(trifluoromethyl)-2-pyridyl]propyl]amino]-2-oxo-acetate (1.38 g, crude) as a yellow oil.

LCMS (ESI): [M+H]⁺m/z: calcd 373.1; found 373.0; Rt=1.241 min.

### Step 5: N′-methyl-N′-[1-[5-(trifluoromethyl)-2-pyridyl]propyl]oxamide

2,2,2-trifluoroethyl 2-[methyl-[1-[5-(trifluoromethyl)-2-pyridyl]propyl]amino]-2-oxo-acetate (1.38 g, 3.71 mmol) was dissolved in Ammonia (7N in methanol, 15,3% w/w)(11.68 g, 686.13 mmol, 15 mL). Resulting reaction mixture was stirred at 25° C. for 18 hr. Then, volatiles were removed under reduced pressure and residue was purified by gradient column chromatography (SiO₂, CHCl₃/ACN), affording N′-methyl-N′-[1-[5-(trifluoromethyl)-2-pyridyl]propyl]oxamide (560 mg, 1.94 mmol, 52.23% yield) as a light-yellow solid.

LCMS (ESI): [M+H]⁺m/z: calcd 290.11; found 290.2; Rt=1.050 min.

### Step 6: N-(4-amino-2-tetrahydropyran-2-yl-pyrazolo[4,3-c]pyridin-7-yl)-N′-methyl-N′-[1-[5-(trifluoromethyl)-2-pyridyl]propyl]oxamide

N′-methyl-N′-[1-[5-(trifluoromethyl)-2-pyridyl]propyl]oxamide (190 mg, 656.86 μmol), 7-bromo-2-tetrahydropyran-2-yl-pyrazolo[4,3-c]pyridin-4-amine (253.74 mg, 853.92 μmol), Copper (4.17 mg, 65.69 μmol), Copper (I) iodide (62.55 mg, 328.43 μmol, 11.13 μL), (S,S)-(+)—N,N′-Dimethyl-1,2-cyclohexanediamine (46.72 mg, 328.43 μmol, 51.79 μL) and Cesium carbonate (321.03 mg, 985.30 μmol) were mixed together in Dioxane (4 mL). Reaction flask was purged with argon and resulting mixture was stirred at 100° C. for 18 hr under inert atmosphere. Then, it was diluted with DCM (15 mL) and filtered. Filtrate was concentrated under reduced pressure, affording N-(4-amino-2-tetrahydropyran-2-yl-pyrazolo[4,3-c]pyridin-7-yl)-N′-methyl-N′-[1-[5-(trifluoromethyl)-2-pyridyl]propyl]oxamide (450 mg, crude) as a brown gum.

LCMS (ESI): [M+H]⁺m/z: calcd 506.21; found 506.2; Rt=0.896 min.

### Step 7: The synthesis of N-(4-amino-1H-pyrazolo[4,3-c]pyridin-7-yl)-N′-methyl-N′-[1-[5-(trifluoromethyl)-2-pyridyl]propyl]oxamide

Hydrogen chloride solution 4.0M in dioxane (1.60 g, 4.39 mmol, 2 mL, 10% purity) was added to the solution of N-(4-amino-2-tetrahydropyran-2-yl-pyrazolo[4,3-c]pyridin-7-yl)-N′-methyl-N′-[1-[5-(trifluoromethyl)-2-pyridyl]propyl]oxamide (450 mg, 498.52 μmol) in Methanol (4 mL). The resulting reaction mixture was stirred at 25° C. for 4 hr. Then, it was concentrated under reduced pressure and residue was purified by HPLC (5-55% 0-5 min H₂O/ACN/0.1% FA, flow: 30 ml/min, column: Chromatorex 18 SMB 100-5T 100×19 mm 5 μm), affording N-(4-amino-1H-pyrazolo[4,3-c]pyridin-7-yl)-N′-methyl-N′-[1-[5-(trifluoromethyl)-2-pyridyl]propyl]oxamide (105 mg, 224.65 μmol, 45.06% yield, HCOOH).

¹H NMR (600 MHz, DMSO-d₆) δ 0.44-0.98 (m, 3H), 1.92-2.09 (m, 1H), 2.12-2.28 (m, 1H), 2.62-3.04 (m, 3H), 5.18-5.63 (m, 1H), 6.64-7.00 (m, 2H), 7.57-7.79 (m, 2H), 8.13-8.31 (m, 2H), 8.70-9.01 (m, 1H), 9.62-10.64 (m, 1H), 12.42-13.46 (m, 1H).

LCMS (ESI): [M+H]⁺m/z: calcd 422.17; found 422.0; Rt=2.241 min.

### Example 146. Synthesis of N-(4-Amino-1H-pyrazolo[4,3-c]pyridin-7-yl)-N′-cyclopropyl-N′-[[4-(trifluoromethyl)phenyl]methyl]oxamide (Compound 107)

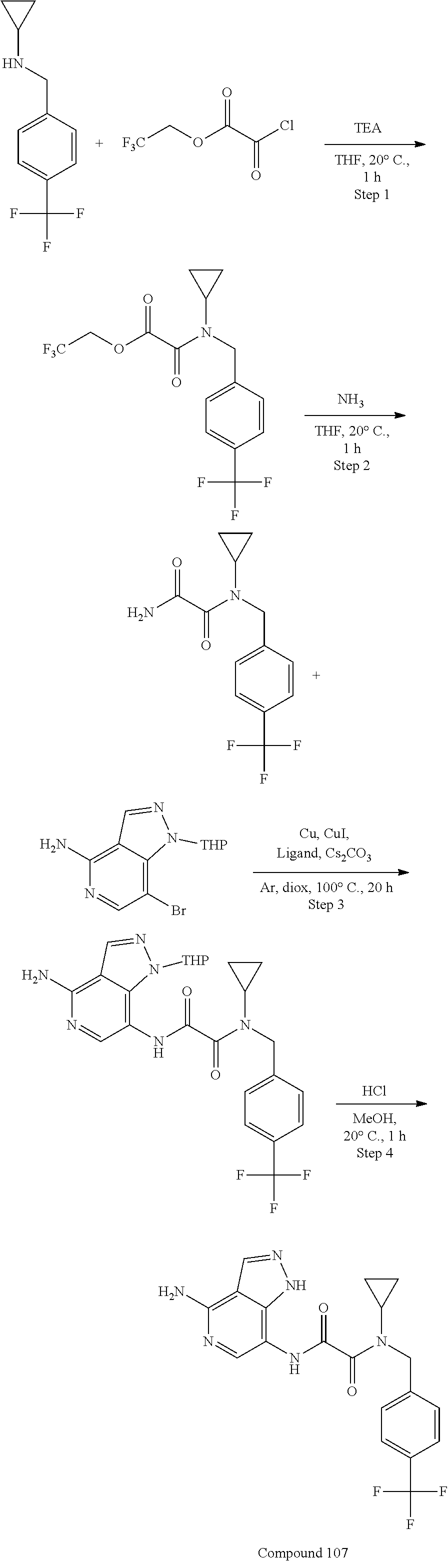

### Step 1: The Synthesis of 2,2,2-Trifluoroethyl 2-[cyclopropyl-[[4-(trifluoromethyl)phenyl]methyl]amino]-2-oxo-acetate

2,2,2-Trifluoroethyl 2-chloro-2-oxo-acetate (0.15 g, 787.38 μmol) was added dropwise to a stirred solution of N-[[4-(trifluoromethyl)phenyl]methyl]cyclopropanamine (154.05 mg, 715.80 μmol) and TEA (86.92 mg, 858.96 μmol, 119.72 μL) in THF (15.23 mL) at 0° C., stirred for 1 hr at 0° C. Reaction mixture was used in the next step.

### Step 2: The Synthesis of N′-Cyclopropyl-N′-[[4-(trifluoromethyl)phenyl]methyl]oxamide

Ammonia (230.60 mg, 13.54 mmol) was bubbled trough a reaction mixture from previous step at 20° C., stirred for 1 hr at 20° C. Reaction mixture was filtered, solid washed with THF (2×5 mL), filtrate concentrated in vacuo to give pure N′-cyclopropyl-N′-[[4-(trifluoromethyl)phenyl]methyl]oxamide (0.18 g, 628.82 μmol, 92.88% yield).

LCMS (ESI): [M+H]⁺m/z: calcd 287.0; found 287.0; Rt=0.958 min.

### Step 3: The Synthesis of N-(4-Amino-2-tetrahydropyran-2-yl-pyrazolo[4,3-c]pyridin-7-yl)-N′-cyclopropyl-N′-[[4-(trifluoromethyl)phenyl]methyl]oxamide

Copper (2.00 mg, 31.44 μmol), Copper (I) iodide (59.88 mg, 314.41 μmol, 10.65 μL), cesium carbonate (307.32 mg, 943.23 μmol) was added to a stirred solution of N′-cyclopropyl-N′-[[4-(trifluoromethyl)phenyl]methyl]oxamide (0.18 g, 628.82 μmol), 7-bromo-2-tetrahydropyran-2-yl-pyrazolo[4,3-c]pyridin-4-amine (186.86 mg, 628.82 μmol), rac-(1R,2R)—N1,N2-dimethylcyclohexane-1,2-diamine (44.72 mg, 314.41 μmol) in 1,4-dioxane (7.00 mL) under Ar atmosphere and stirred at 110° C. for 48 hr in closed vial. RM was cooled to r.t., filtered, solid washed with dioxane (2×3 mL), filtrate used in the next step.

LCMS (ESI): [M+H]⁺m/z: calcd 503.4; found 503.4; Rt=1.173 min.

### Step 4: The Synthesis of N-(4-Amino-1H-pyrazolo[4,3-c]pyridin-7-yl)-N′-cyclopropyl-N′-[[4-(trifluoromethyl)phenyl]methyl]oxamide

Hydrogen chloride solution 4.0M in dioxane (1.09 g, 29.85 mmol, 1.36 mL) was added to a solution of N-(4-amino-2-tetrahydropyran-2-yl-pyrazolo[4,3-c]pyridin-7-yl)-N′-cyclopropyl-N′-[[4-(trifluoromethyl)phenyl]methyl]oxamide (0.3 g, 597.03 μmol) in Methanol (2 mL) and stirred at 20° C. for 1 hr. Volatiles was evaporated in vacuo, the residue triturated with IPA (5 mL), filtered, washed with IPA (5 mL), and submitted to HPLC (column: Chromatorex 18 SMB100-5T 100×19 mm 5 μm; Oct. 10, 2030% 0-1.3-5.3 min H₂O/ACN/0.1% FA, flow rate: 30 mL/min) to give N-(4-amino-1H-pyrazolo[4,3-c]pyridin-7-yl)-N′-cyclopropyl-N′-[4-(trifluoromethyl)phenyl]methyl]oxamide (28 mg, 60.29 μmol, 10.10% yield, HCOOH).

¹H NMR (600 MHz, dmso) 8 0.62-0.87 (m, 4H), 2.81-2.88 (m, 1H), 4.66-4.85 (m, 2H), 6.59-7.01 (m, 2H), 7.43-7.72 (m, 3H), 7.73-7.79 (m, 2H), 8.17-8.24 (m, 1H), 9.69-10.54 (m, 1H), 12.52-13.31 (m, 1H).

LCMS (ESI): [M+H]⁺m/z: calcd 419.0; found 419.0; Rt=2.384 min.

### Example 147. Synthesis of (R)—N¹-(4-amino-1H-pyrazolo[4,3-c]pyridin-7-yl)-N2-(1-(2-fluorophenyl)ethyl)-N2-methyloxalamide (Compound 162)

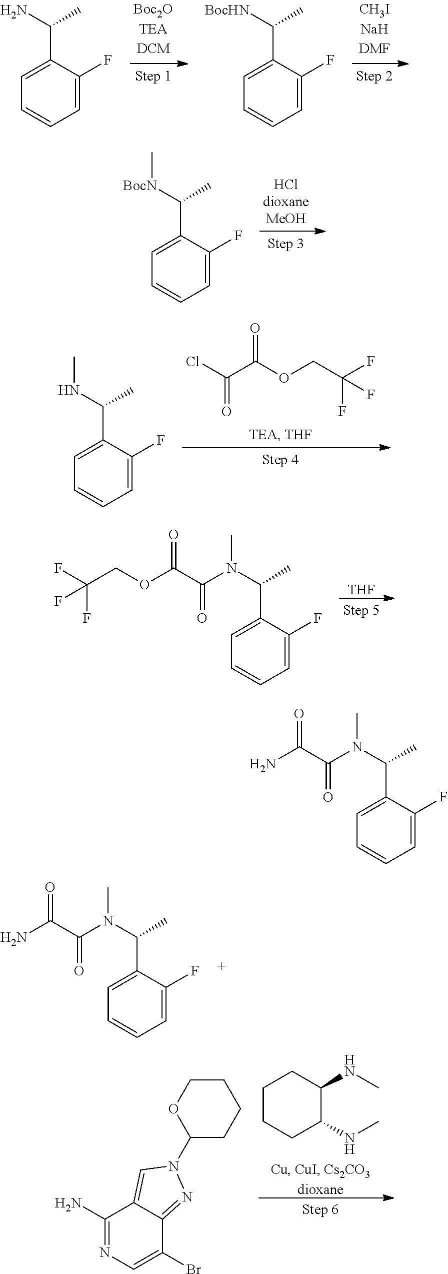

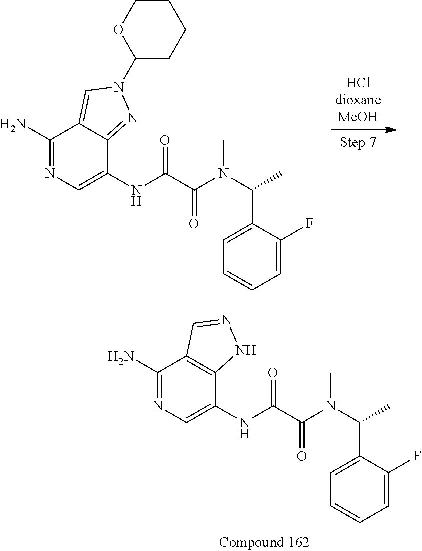

### Step 1: (R)-tert-butyl(1-(2-fluorophenyl)ethyl)carbamate

To a solution of (R)-1-(2-fluorophenyl)ethanamine (0.7 g, 3.99 mmol, HCl) and Triethylamine (604.96 mg, 5.98 mmol, 833.28 μL) in DCM (20 mL), Di-tert-butyl dicarbonate (826.37 mg, 3.79 mmol, 868.95 μL) was added. The resulting mixture was stirred at 25° C. for 3 hr, washed with water (3×20 mL), dried over anhydrous sodium sulfate and evaporated in vacuo to give (R)-tert-butyl(1-(2-fluorophenyl)ethyl)carbamate (0.92 g, 3.84 mmol, 96.47% yield) as a light-yellow oil.

LCMS (ESI): [M-t-Bu]; m/z: calcd 184.1; found 184.1; Rt=1.492 min.

### Step 2: (R)-tert-butyl(1-(2-fluorophenyl)ethyl)(methyl)carbamate

To a stirred at 0° C. solution of (R)-tert-butyl(1-(2-fluorophenyl)ethyl)carbamate (920 mg, 3.84 mmol) in DMF (15 mL), Sodium Hydride (in oil dispersion) 60% dispersion in mineral oil (307.58 mg, 7.69 mmol, 60% purity) was added. After 30 min, Methyl iodide (1.09 g, 7.69 mmol, 478.71 μL) was added. The resulting mixture was stirred at 25° C. for 6 hr and poured into water (80 mL) and extracted with MTBE (3×15 mL), dried over anhydrous sodium sulfate and evaporated in vacuo to obtain (R)-tert-butyl(1-(2-fluorophenyl)ethyl)(methyl)carbamate (0.9 g, 3.55 mmol, 92.41% yield) as a light-yellow oil.

LCMS (ESI): [M-t-Bu]-m/z: calcd 198.2; found 198.2; Rt=1.579 min.

### Step 3: (R)-1-(2-fluorophenyl)-N-methylethanamine

A solution of (R)-tert-butyl(1-(2-fluorophenyl)ethyl)(methyl)carbamate (900 mg, 3.55 mmol) in MeOH (20 mL) and Hydrogen chloride solution 4.0M in dioxane (8.00 g, 219.41 mmol, 10 mL) was stirred at 25° C. for 3 hr. The solvent was evaporated to (R)-1-(2-fluorophenyl)-N-methylethanamine (0.65 g, 3.43 mmol, 96.46% yield, HCl) as a light-yellow solid.

LCMS (ESI): [M+H]⁺m/z: calcd 154.11; found 154.0; Rt=0.501 min.

### Step 4: (R)-2,2,2-trifluoroethyl 2-((1-(2-fluorophenyl)ethyl)(methyl)amino)-2-oxoacetate

To a solution of (R)-1-(2-fluorophenyl)-N-methylethanamine (0.5 g, 2.64 mmol, HCl) and Triethylamine (1.07 g, 10.55 mmol, 1.47 mL) in THF (30.44 mL), 2,2,2-trifluoroethyl 2-chloro-2-oxo-acetate (1.26 g, 6.59 mmol) was added portionwise at 0° C. The resulting mixture was stirred for 3 hr. LCMS showed full conversion of SM. The reaction mixture was directly used for the next step.

LCMS (ESI): [M+H]⁺m/z: calcd 308.09; found 308.0; Rt=1.281 min.

### Step 5: (R)—N¹-(1-(2-fluorophenyl)ethyl)-N1-methyloxalamide

Through a solution of (R)-2,2,2-trifluoroethyl 2-((1-(2-fluorophenyl)ethyl)(methyl)amino)-2-oxoacetate (0.8 g, 2.60 mmol) in THF (30 mL), ammonia was bubbled during 10 min at 0° C. The formed precipitate was filtered off, washed with THE (30 mL) and the solvent was evaporated in vacuo to give crude product (1 g), which was purified by gradient chromatography (CHCl₃-ACN) to afford (R)—N¹-(1-(2-fluorophenyl)ethyl)-N1-methyloxalamide (0.3 g, 1.34 mmol, 51.38% yield) as a light-yellow solid.

LCMS (ESI): [M−H]⁻ m/z: calcd 223.09; found 223.8; Rt=1.142 min.

### Step 6: N1-(4-amino-2-(tetrahydro-2H-pyran-2-yl)-2H-pyrazolo[4,3-c]pyridin-7-yl)-N2-((R)-1-(2-fluorophenyl)ethyl)-N2-methyloxalamide

To a mixture of (R)—N¹-(1-(2-fluorophenyl)ethyl)-N1-methyloxalamide (0.2 g, 891.94 μmol), 7-bromo-2-tetrahydropyran-2-yl-pyrazolo[4,3-c]pyridin-4-amine (331.30 mg, 1.11 mmol), Copper (11.34 mg, 178.39 μmol), Copper (I) iodide (169.87 mg, 891.94 μmol, 30.23 μL), Cesium carbonate (581.22 mg, 1.78 mmol) and rac-(1R,2R)—N1,N2-dimethylcyclohexane-1,2-diamine (190.30 mg, 1.34 mmol), Dioxane (4 mL) was added. The resulting mixture was evacuated, refiled with Argon three time, heated at 100° C. for 18 hr and cooled. The inorganic precipitate was filtered off and washed with DCM (30 mL) to give N1-(4-amino-2-(tetrahydro-2H-pyran-2-yl)-2H-pyrazolo[4,3-c]pyridin-7-yl)-N2-((R)-1-(2-fluorophenyl)ethyl)-N2-methyloxalamide (0.4 g, crude).

This substance was used for the next step without further purification.

LCMS (ESI): [M+H]⁺m/z: calcd 441.21; found 441.2; Rt=1.066 min.

### Step 7: The synthesis of (R)—N¹-(4-amino-1H-pyrazolo[4,3-c]pyridin-7-yl)-N2-(1-(2-fluorophenyl)ethyl)-N2-methyloxalamide

To a solution of N1-(4-amino-2-(tetrahydro-2H-pyran-2-yl)-2H-pyrazolo[4,3-c]pyridin-7-yl)-N2-((R)-1-(2-fluorophenyl)ethyl)-N2-methyloxalamide (0.4 g, 908.12 μmol) in MeOH (10 mL), Hydrogen chloride solution 4.0M in dioxane (3 g, 82.28 mmol) was added. The resulting mixture was stirred at 25° C. for 3 hr and evaporated in vacuo. The residue was purified by HPLC (Device (Mobile Phase, Column): SYSTEM 5-30% 0-5 min H₂O/ACN/0.1% FA, flow: 30 mL/min (loading pump 4 mL/min H₂O) target mass 356.36 column: Chromatorex 18 SMB100-5T 100×19 mm 5 μm) to obtain (R)—N¹-(4-amino-1H-pyrazolo[4,3-c]pyridin-7-yl)-N2-(1-(2-fluorophenyl)ethyl)-N2-methyloxalamide (235 mg, 659.46 μmol, 72.62% yield) as a light-yellow solid.

¹H NMR (600 MHz, DMSO-d₆) § 1.08-1.67 (m, 3H), 2.58-2.91 (m, 3H), 5.42-5.93 (m, 1H), 6.61-6.84 (m, 2H), 7.14-7.29 (m, 2H), 7.33-7.43 (m, 1H), 7.52 (td, 1H), 7.64-7.82 (m, 1H), 8.19 (d, 1H), 9.46-10.59 (m, 1H), 12.17-13.54 (m, 1H).

LCMS (ESI): [M+H]⁺m/z: calcd 357.16; found 357.2; Rt=1.739 min.

### Example 148. Synthesis of N-(4-Amino-1H-pyrazolo[4,3-c]pyridin-7-yl)-N′-cyclobutyl-N′-[[4-(trifluoromethyl)phenyl]methyl]oxamide (Compound 118)

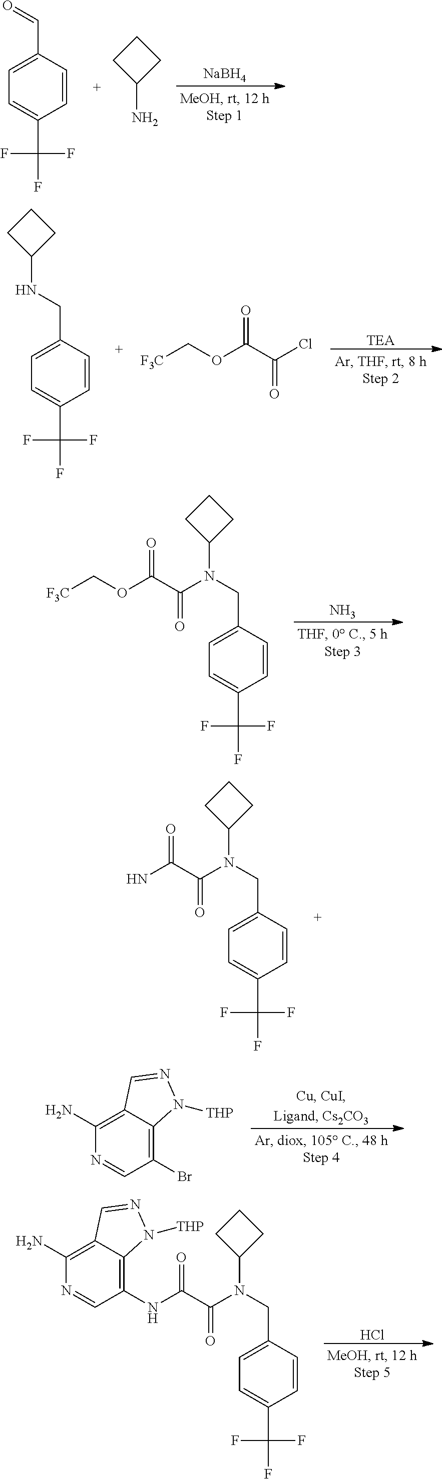

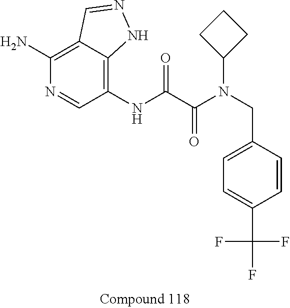 [SMILES: Nc1ncc(NC(=O)C(=O)N(Cc2ccc(C(F)(F)F)cc2)C2CCC2)c2[nH]ncc12]

### Step 1: The Synthesis of N-[[4-(Trifluoromethyl)phenyl]methyl]cyclobutanamine

A solution of 4-(trifluoromethyl)benzaldehyde (0.8 g, 4.59 mmol, 627.45 μL) and cyclobutanamine (392.12 mg, 5.51 mmol, 470.73 μL) in MeOH (9.40 mL) was stirred at 20° C. for 12 h. To this solution, Sodium Borohydride (347.62 mg, 9.19 mmol, 323.67 μL) was added and the resulting mixture was stirred for 5 hr. The solvent was removed in vacuo, the residue was taken up with water (30 mL) and extracted with DCM (2*20 mL). The combined organic layer was washed with brine (10 mL), dried over Na₂SO₄ and evaporated to obtain N-[4-(trifluoromethyl)phenyl]methyl]cyclobutanamine (0.32 g, 1.40 mmol, 30.38% yield). ¹H NMR (400 MHz, CDCl₃) δ (ppm) 1.67 (m, 7H), 2.22 (m, 2H), 3.28 (m, 1H), 7.42 (d, 2H), 7.54 (d, 2H).

### Step 2: The Synthesis of 2,2,2-Trifluoroethyl 2-[cyclobutyl-[[4-(trifluoromethyl)phenyl]methyl]amino]-2-oxo-acetate

To a solution of N-[4-(trifluoromethyl)phenyl]methyl]cyclobutanamine (0.38 g, 1.66 mmol) and TEA (251.61 mg, 2.49 mmol, 346.56 μL) in THF (15 mL) was added 2,2,2-trifluoroethyl 2-chloro-2-oxo-acetate (378.95 mg, 1.99 mmol) dropwise at 0° C. under argon. The reaction mixture was then stirred for 8 hr at r.t., then evaporated in vacuo, the residue was taken up with water (30 mL) and extracted with DCM (3*20 mL). The combined organic layer was washed with brine (10 mL), dried over Na₂SO₄ and evaporated to obtain 2,2,2-trifluoroethyl 2-[cyclobutyl-[4-(trifluoromethyl)phenyl]methyl]amino]-2-oxo-acetate (0.6 g, 1.57 mmol, 94.44% yield).

### Step 3: The Synthesis of N′-Cyclobutyl-N′-[[4-(trifluoromethyl)phenylmethyl]oxamide

2,2,2-Trifluoroethyl 2-[cyclobutyl-[[4-(trifluoromethyl)phenyl]methyl]amino]-2-oxo-acetate (0.6 g, 1.57 mmol) was dissolved in THF (20 mL) and was blow ammonium (598.28 mg, 35.13 mmol). Resulting solution was stirred at 0° C. for 5 hr. The resulting mixture was evaporated in vacuo and residue was triturated with THF (20 mL*2), filtered and combined organic was evaporated in vacuo to leave 0.4 g of crude product, 0.4 g of which was purification by column chromatography on silica gel using CHCl₃/CH₃CN gradient (10-100% CH₃CN) to afford N′-cyclobutyl-N′-[4-(trifluoromethyl)phenyl]methyl]oxamide (0.2 g, 666.05 μmol, 42.55% yield).

LCMS (ESI): [M+H]⁺m/z: calcd 301.0; found 301.0; Rt=1.141 min.

### Step 4: The Synthesis of N-(4-Amino-1-tetrahydropyran-2-yl-pyrazolo[4,3-c]pyridin-7-yl)-N′-cyclobutyl-N′-[[4-(trifluoromethyl)phenyl]methyl]oxamide

7-Bromo-1-tetrahydropyran-2-yl-pyrazolo[4,3-c]pyridin-4-amine (148.44 mg, 499.54 μmol), N′-cyclobutyl-N′-[4-(trifluoromethyl)phenyl]methyl]oxamide (0.1 g, 333.03 μmol), Cul (97.08 mg, 509.73 μmol, 17.27 μL), cesium carbonate (162.76 mg, 499.54 μmol), (1R,2R)—N1,N2-dimethylcyclohexane-1,2-diamine (97.08 mg, 682.49 μmol) and Cu(1.06 mg, 16.65 μmol) were mixed in dioxane (6.01 mL), purged with Ar for 15 minutes and then heated in the sealed tube at 105° C. for 48 hr. Final mixture was filtered and dioxane was evaporated in vacuo. The crude product 0.3 g was purified by RP-HPLC (column: XBridge C18 5 μm 130 A; 40-90% 0-1-5 min H₂O/CH₃OH/0.1% NH₄OH, flow: 30 mL/min) to give N-(4-amino-1-tetrahydropyran-2-yl-pyrazolo[4,3-c]pyridin-7-yl)-N′-cyclobutyl-N′-[4-(trifluoromethyl)phenyl]methyl]oxamide (0.052 g, 100.67 μmol, 30.23% yield).

LCMS (ESI): [M+H]⁺m/z: calcd 517.2; found 517.2; Rt=2.978 min.

### Step 5: The Synthesis of N-(4-Amino-1H-pyrazolo[4,3-c]pyridin-7-yl)-N′-cyclobutyl-N′-[4-(trifluoromethyl)phenyl]methyl]oxamide (Compound 118)

Hydrogen chloride solution 4.0M in dioxane (7.45 mg, 204.33 μmol, 5 mL) was added to a solution of N-(4-amino-1-tetrahydropyran-2-yl-pyrazolo[4,3-c]pyridin-7-yl)-N′-cyclobutyl-N′-[4-(trifluoromethyl)phenyl]methyl]oxamide (105.54 mg, 204.33 μmol) in MeOH (15 mL). The reaction mixture was stirred at 20° C. for 12 hr, then evaporated was purified by RP-HPLC (column: XBridge BEH18 SMB100-BT 100*19 mm; 55-55-100% 0-1-6 min H₂O/CH₃OH/0.1% NH₄OH, flow: 30 mL/min) to give N-(4-amino-1H-pyrazolo[4,3-c]pyridin-7-yl)-N′-cyclobutyl-N′-[4-(trifluoromethyl)phenyl]methyl]oxamide (9.70 mg, 22.43 μmol, 10.98% yield).

¹H NMR (600 MHz, dmso) δ 1.52-1.63 (m, 2H), 1.83-2.05 (m, 2H), 2.09-2.27 (m, 2H), 4.41-5.13 (m, 3H), 6.43-6.70 (m, 2H), 7.00-7.43 (m, 1H), 7.47-7.56 (m, 2H), 7.64-7.74 (m, 2H), 8.09-8.24 (m, 1H), 9.67-10.60 (m, 1H), 12.32-13.67 (m, 1H).

LCMS (ESI): [M+H]⁺m/z: calcd 433.0; found 433.0; Rt=2.473 min.

### Example 149. Synthesis of N-(4-amino-1H-pyrazolo[4,3-c]pyridin-7-yl)-N′-isobutyl-N′-[[4-(pentafluoro-sulfanyl)phenyl]methyl]oxamide (Compound 110)

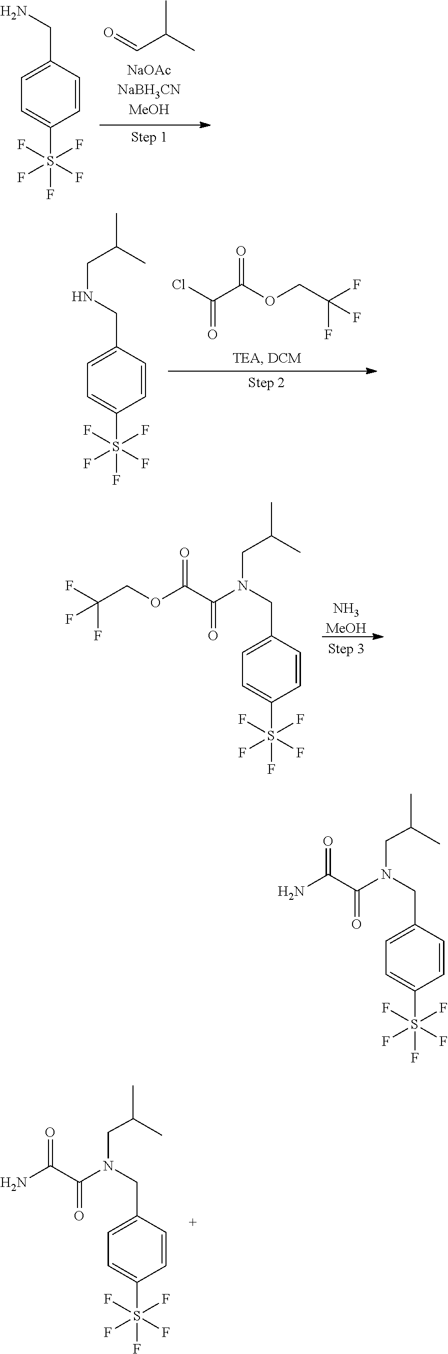

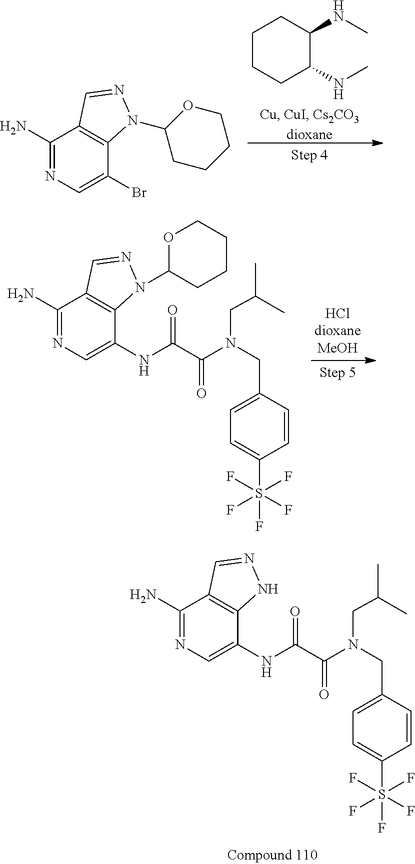

### Step 1:2-methyl-N-[[4-(pentafluoro-sulfanyl)phenyl]methyl]propan-1-amine

To a solution of [4-(pentafluoro-sulfanyl)phenyl]methanamine (0.3 g, 1.11 mmol, HCl) and 2-methylpropanal (96.26 mg, 1.34 mmol, 121.24 μL) in Methanol (10 mL) was added Potassium Acetate (163.77 mg, 1.67 mmol, 104.31 μL) and Sodium cyanoborohydride (104.86 mg, 1.67 mmol). The mixture was stirred at 20° C. for 12 hr. The reaction was quenched by adding water (10 mL) and was extracted with EtOAc (2×10 mL). The combined organic layers were washed with brine (2×50 mL), dried over anhydrous sodium sulfate and concentrated under reduced pressure to give 2-methyl-N-[[4-(pentafluoro-sulfanyl)phenyl]methyl]propan-1-amine (301 mg, crude) as a light-yellow oil.

LCMS (ESI): [M+H]⁺m/z: calcd 290.1; found 290.2; Rt=0.879 min.

### Step 2:2,2,2-trifluoroethyl 2-[isobutyl-[4-(pentafluoro-sulfanyl)phenyl]methyl]amino]-2-oxo-acetate

To a stirred solution of 2-methyl-N-[4-(pentafluoro-sulfanyl)phenyl]methyl]propan-1-amine (340 mg, 1.18 mmol) in Dichloromethane (3.5 mL) were added triethylamine (178.38 mg, 1.76 mmol, 245.70 μL) respectively at room temperature. The resulting reaction mixture was cooled to 0° C. Then 2,2,2-trifluoroethyl 2-chloro-2-oxo-acetate (223.89 mg, 1.18 mmol) was added dropwise. The reaction was stirred extra 30 minutes at 0° C., then allowed warmed to room temperature and stirred 15 hr. Upon completion, the reaction mixture was washed with water (2×10 mL). Organic phase was then dried over anhydrous sodium sulfate, filtered and concentrated under reduced pressure to obtain 2,2,2-trifluoroethyl 2-[isobutyl-[[4-(pentafluoro-sulfanyl)phenyl]methyl]amino]-2-oxo-acetate (355 mg, crude) was isolated as a brown gum.

LCMS (ESI): [M+H]⁺m/z: calcd 444.09; found 444.2; Rt=1.537 min.

### Step 3: N′-isobutyl-N′-[4-(pentafluoro-sulfanyl)phenyl]methyl]oxamide

A solution of 2,2,2-trifluoroethyl 2-[isobutyl-[[4-(pentafluoro-sulfanyl)phenyl]methyl]amino]-2-oxo-acetate (355 mg, 800.72 μmol) in Methanol/NH3 (7N)(10 mL) was stirred at 25° C. for 15 hr. The solvent was evaporated to obtain N′-isobutyl-N′-[[4-(pentafluoro-sulfanyl)phenyl]methyl]oxamide (256 mg, crude) as a yellow gum.

LCMS (ESI): [M+H]⁺m/z: calcd 361.1; found 361.1; Rt=1.436 min.

### Step 4: N-(4-amino-1-tetrahydropyran-2-yl-pyrazolo[4,3-c]pyridin-7-yl)-N′-isobutyl-N′-[[4-(pentafluoro-sulfanyl)phenyl]methyl]oxamide

Copper (2.26 mg, 35.52 μmol), Copper (I) iodide (67.65 mg, 355.22 μmol, 12.04 μL), cesium carbonate (347.21 mg, 1.07 mmol) was added to a stirred solution of N′-isobutyl-N′-[[4-(pentafluoro-sulfanyl)phenyl]methyl]oxamide (256 mg, 710.44 μmol), 7-bromo-1-tetrahydropyran-2-yl-pyrazolo[4,3-c]pyridin-4-amine (211.11 mg, 710.44 μmol), rac-(1R,2R)—N1,N2-dimethylcyclohexane-1,2-diamine (50.53 mg, 355.22 μmol) in 1,4-dioxane (5 mL) under Ar atmosphere and stirred at 90° C. for 48 hr in closed vial. The reaction mixture was cooled and filtered. The filtercake was washed with MeOH (10 mL) and the filtrate was concentrated in vacuo to obtain N-(4-amino-1-tetrahydropyran-2-yl-pyrazolo[4,3-c]pyridin-7-yl)-N′-isobutyl-N′-[[4-(pentafluoro-sulfanyl)phenyl]methyl]oxamide (580 mg, crude) as a brown solid which was used in the next step without purification.

LCMS (ESI): [M+H]⁺m/z: calcd 577.2; found 577.4; Rt=1.148 min.

### Step 5: The synthesis of N-(4-amino-1H-pyrazolo[4,3-c]pyridin-7-yl)-N′-isobutyl-N′-[[4-(pentafluoro-sulfanyl)phenyl]methyl]oxamide

N-(4-amino-1-tetrahydropyran-2-yl-pyrazolo[4,3-c]pyridin-7-yl)-N′-isobutyl-N′-[4-(pentafluoro-sulfanyl)phenyl]methyl]oxamide (580 mg, 1.01 mmol) was dissolved in Dioxane/HCl (2 mL) and Methanol (2 mL). The resulting solution was stirred at rt for 15 hr. Solvents were evaporated. Resulting crude product was purified by HPLC (Device (Mobile Phase, Column): SYSTEM; 0-2-10 min0-55% H₂O/ACN/0.1FA, flow 30 mL/min ((loading pump 4 mL ACN); target mass 492; column: Chromatorex C18 SMB100-5T 100×19 mm, 5 microM) to obtain N-(4-amino-1H-pyrazolo[4,3-c]pyridin-7-yl)-N′-isobutyl-N′-[4-(pentafluoro-sulfanyl)phenyl]methyl]oxamide (37.6 mg, 69.82 μmol, 6.94% yield, HCOOH) as a light-yellow solid.

¹H NMR (600 MHz, DMSO-d₆) δ 0.32-0.88 (m, 6H), 1.81-2.06 (m, 1H), 3.10-3.13 (m, 1H), 3.38-3.39 (m, 1H), 4.45-4.93 (m, 2H), 6.51-6.85 (m, 2H), 7.55-7.70 (m, 2H), 7.81-7.92 (m, 2H), 8.10-8.20 (m, 2H), 8.53-10.62 (m, 1H), 12.45-13.39 (m, 1H).

LCMS (ESI): [M+H]⁺m/z: calcd 493.17; found 493.0; Rt=3.115 min.

### Example 150. Synthesis of (R)—N¹-(4-amino-1H-pyrazolo[4,3-c]pyridin-7-yl)-N2-(1-(4-fluorophenyl)ethyl)-N2-methyloxalamide (Compound 144)

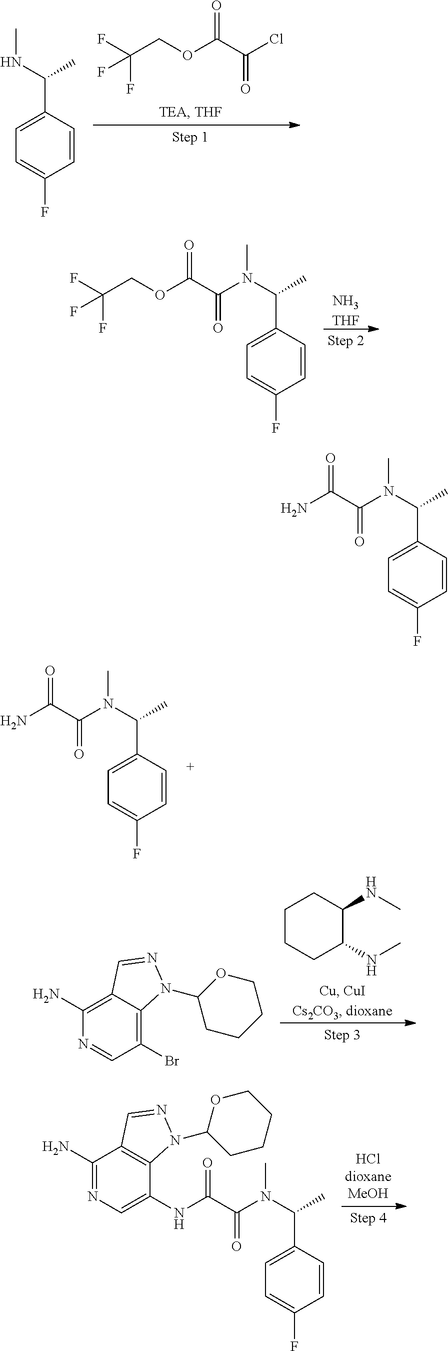

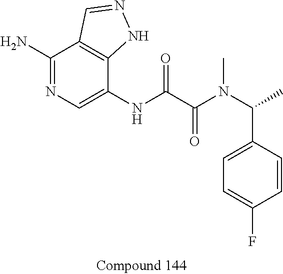 [SMILES: C[C@H](c1ccc(F)cc1)N(C)C(=O)C(=O)Nc1cnc(N)c2cn[nH]c12]

### Step 1: (R)-2,2,2-trifluoroethyl 2-((1-(4-fluorophenyl)ethyl)(methyl)amino)-2-oxoacetate

To a solution of (R)-1-(4-fluorophenyl)-N-methylethanamine (0.5 g, 3.26 mmol) and TEA (495.39 mg, 4.90 mmol, 682.36 μL) in THE (30.05 mL) was added 2,2,2-trifluoroethyl 2-chloro-2-oxo-acetate (746.12 mg, 3.92 mmol) dropwise at 0° C. under argon. The reaction mixture was then stirred for 8 hr at r.t., then evaporated in vacuo, the residue was taken up with water (40 mL) and extracted with DCM (3×20 mL). The combined organic layer was washed with brine (10 mL), dried over anhydrous sodium sulfate and evaporated to obtain (R)-2,2,2-trifluoroethyl 2-((1-(4-fluorophenyl)ethyl)(methyl)amino)-2-oxoacetate (0.65 g, 2.12 mmol, 64.82% yield) as a light-yellow oil.

### Step 2: (R)—N¹-(1-(4-fluorophenyl)ethyl)-N1-methyloxalamide

(R)-2,2,2-trifluoroethyl 2-((1-(4-fluorophenyl)ethyl)(methyl)amino)-2-oxoacetate (0.65 g, 2.12 mmol) was dissolved in THF (20 mL) and was blow ammonium (763.31 mg, 44.82 mmol). Resulting solution was stirred at 0° C. for 5 hr. The resulting mixture was evaporated in vacuo and residue was triturated with THF (2×20 mL), filtered and combined organic was evaporated in vacuo to leave 0.6 g of crude product, 0.6 g of which was purification by column chromatography on silica gel using CHCl₃/MTBE gradient (10-100% MTBE) to afford (R)—N¹-(1-(4-fluorophenyl)ethyl)-N1-methyloxalamide (0.2 g, 891.94 μmol, 42.16% yield) as a light-yellow solid.

### Step 3: N1-(4-amino-1-(tetrahydro-2H-pyran-2-yl)-1H-pyrazolo[4,3-c]pyridin-7-yl)-N2-((R)-1-(4-fluorophenyl)ethyl)-N2-methyloxalamide

7-bromo-1-tetrahydropyran-2-yl-pyrazolo[4,3-c]pyridin-4-amine (198.78 mg, 668.95 μmol), (R)—N¹-(1-(4-fluorophenyl)ethyl)-N1-methyloxalamide (0.1 g, 445.97 μmol), CuI (0.130 g, 682.59 μmol, 23.13 μL), cesium carbonate (217.96 mg, 668.95 μmol), (1R,2R)—N1,N2-dimethylcyclohexane-1,2-diamine (0.13 g, 913.95 μmol) and Cu(1.42 mg, 22.30 μmol) were mixed in dioxane (6 mL), purged with Ar for 15 minutes and then heated in the sealed tube at 105° C. for 48 hr. Final mixture was filtered and dioxane was evaporated in vacuo. The crude product 0.3 g was purified by RP-HPLC (column: XBridge C18 5 μm 130A; 40-40-60% 0-1.5-5 min H₂O/CH₃OH/0.1% NH₄OH, flow: 30 mL/min) to give N1-(4-amino-1-(tetrahydro-2H-pyran-2-yl)-1H-pyrazolo[4,3-c]pyridin-7-yl)-N2-((R)-1-(4-fluorophenyl)ethyl)-N2-methyloxalamide (0.09 g, 204.33 μmol, 45.82% yield) as a brown solid.

LCMS (ESI): [M+H]⁺m/z: calcd 441.23; found 441.2; Rt=2.743 min.

### Step 4: The synthesis of (R)—N¹-(4-amino-1H-pyrazolo[4,3-c]pyridin-7-yl)-N2-(1-(4-fluorophenyl)ethyl)-N2-methyloxalamide

Hydrogen chloride solution 4.0M in dioxane (7.45 mg, 204.33 μmol, 5 mL) was added to a solution of N1-(4-amino-1-(tetrahydro-2H-pyran-2-yl)-1H-pyrazolo[4,3-c]pyridin-7-yl)-N2-((R)-1-(4-fluorophenyl)ethyl)-N2-methyloxalamide (0.09 g, 204.33 μmol) in MeOH (15 mL). The reaction mixture was stirred at 20° C. for 12 hr, then evaporated was purified by RP-HPLC (column: XBridge BEH18 SMB100-BT 100×19 mm; 10-45% 0-1-5 min H₂O/CH₃CN/0.1% NH₄OH, flow: 30 mL/min) to give (R)—N¹-(4-amino-1H-pyrazolo[4,3-c]pyridin-7-yl)-N2-(1-(4-fluorophenyl)ethyl)-N2-methyloxalamide (31.10 mg, 87.27 μmol, 42.71% yield) as a light-yellow solid.

¹H NMR (500 MHz, DMSO-d₆) δ 1.23-1.62 (m, 3H), 2.52-2.86 (m, 3H), 4.94-5.82 (m, 1H), 6.61-6.92 (m, 2H), 6.96-7.24 (m, 2H), 7.37-7.44 (m, 1H), 7.44-7.51 (m, 1H), 7.64-7.76 (m, 1H), 8.11-8.25 (m, 1H), 9.46-10.88 (m, 1H), 12.47-13.32 (m, 1H).

LCMS (ESI): [M+H]⁺m/z: calcd 357.16; found 357.2; Rt=1.111 min.

### Example 151. Synthesis of N-(4-amino-1H-pyrazolo[4,3-c]pyridin-7-yl)-N′-ethyl-N′-[1-[4-(pentafluoro-sulfanyl)phenyl]ethyl]oxamide (Compound 140)

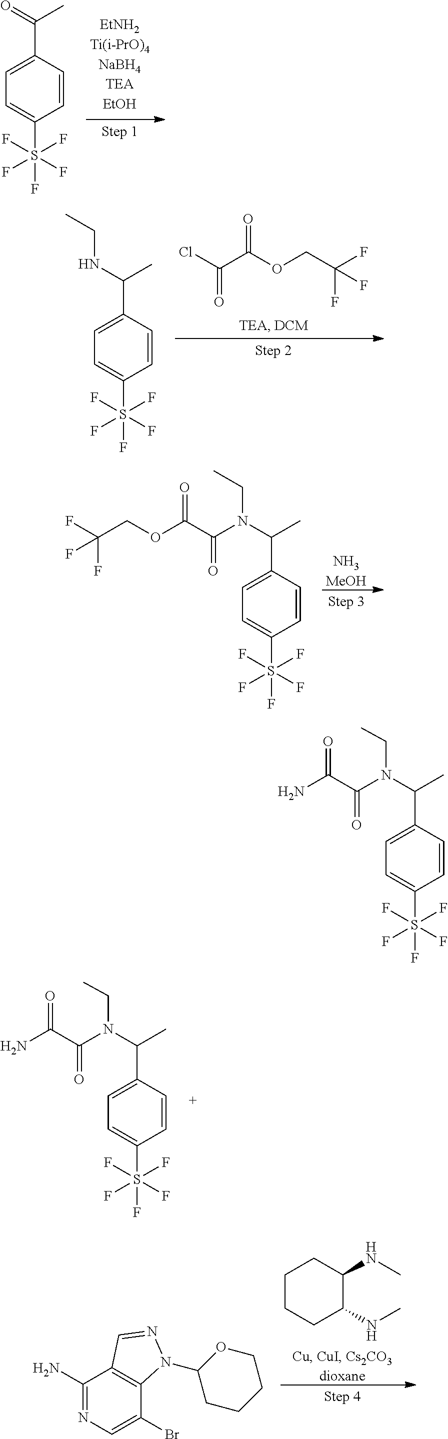

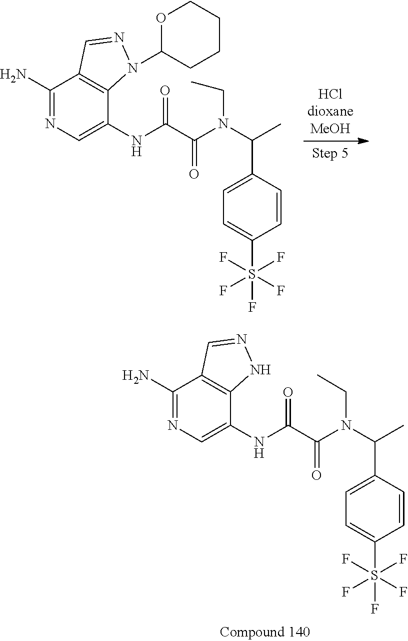

### Step 1: N-ethyl-1-[4-(pentafluoro-sulfanyl)phenyl]ethanamine

A mixture of the 1-[4-(pentafluoro-sulfanyl)phenyl]ethanone (150 mg, 609.27 μmol), titanium isopropoxide (259.74 mg, 913.90 μmol, 271.98 μL), ethanamine (74.52 mg, 913.90 μmol, 92.81 μL, HCl) and TEA (92.48 mg, 913.90 μmol, 127.38 μL) in Ethanol (9 mL) was stirred under Ar at ambient temperature for 15 hr. Sodium Borohydride (23.05 mg, 609.27 μmol, 21.46 μL) was then added and the resulting mixture was stirred for an additional 1 hr at ambient temperature. The reaction was then quenched by pouring into aqueous ammonia (10 mL), the resulting inorganic precipitate was filtered off, and washed with dichloromethane (10 mL). The organic layer was separated, and the remaining aqueous layer was extracted once with dichloromethane. Organic phase was combined, dried over anhydrous sodium sulfate, filtered and evaporated to obtain N-ethyl-1-[4-(pentafluoro-sulfanyl)phenyl]ethanamine (67 mg, crude) as a yellow oil.

LCMS (ESI): [M+H]⁺m/z: calcd 276.09; found 276.0; Rt=1.058 min.

### Step 2:2,2,2-trifluoroethyl 2-[ethyl-[1-[4-(pentafluoro-sulfanyl)phenyl]ethyl]amino]-2-oxo-acetate

To a stirred solution of N-ethyl-1-[4-(pentafluoro-sulfanyl)phenyl]ethanamine (0.101 g, 366.90 μmol) in Dichloromethane (1 mL) were added triethylamine (55.69 mg, 550.35 μmol, 76.71 μL) respectively at room temperature. The resulting reaction mixture was cooled to 0° C. Then 2,2,2-trifluoroethyl 2-chloro-2-oxo-acetate (69.90 mg, 366.90 μmol) was added dropwise. The reaction was stirred extra 30 minutes at 0° C., then allowed warmed to room temperature and stirred 15 hr. Upon completion, the reaction mixture was washed with water (2×20 mL). Organic phase was then dried over anhydrous sodium sulfate, filtered and concentrated under reduced pressure to obtain 2,2,2-trifluoroethyl 2-[ethyl-[1-[4-(pentafluoro-sulfanyl)phenyl]ethyl]amino]-2-oxo-acetate (132 mg, crude) as a brown gum.

LCMS (ESI): [M+H]⁺m/z: calcd 430.07; found 430.2; Rt=1.481 min.

### Step 3: N′-ethyl-N′-[1-[4-(pentafluoro-sulfanyl)phenyl]ethyl]oxamide

A solution of 2,2,2-trifluoroethyl 2-[ethyl-[1-[4-(pentafluoro-sulfanyl)phenyl]ethyl]amino]-2-oxo-acetate (132 mg, 307.46 μmol) in Methanol/NH3 (7N)(3 mL) was stirred at 25° C. for 15 hr. The solvent was evaporated to obtain N′-ethyl-N′-[1-[4-(pentafluoro-sulfanyl)phenyl]ethyl]oxamide (105 mg, crude) as a yellow gum.

LCMS (ESI): [M+Na]⁺m/z: calcd 369.09; found 369.0; Rt=1.350 min.

### Step 4: N-(4-amino-1-tetrahydropyran-2-yl-pyrazolo[4,3-c]pyridin-7-yl)-N′-ethyl-N′-[1-[4-(pentafluoro-sulfanyl)phenyl]ethyl]oxamide

Copper (963.39 μg, 15.16 μmol), Copper (I) iodide (28.87 mg, 151.60 μmol, 5.14 μL), cesium carbonate (148.18 mg, 454.79 μmol) was added to a stirred solution of N′-ethyl-N′-[1-[4-(pentafluoro-sulfanyl)phenyl]ethyl]oxamide (105 mg, 303.19 μmol), 7-bromo-1-tetrahydropyran-2-yl-pyrazolo[4,3-c]pyridin-4-amine (90.09 mg, 303.19 μmol), rac-(1R,2R)—N1,N2-dimethylcyclohexane-1,2-diamine (21.56 mg, 151.60 μmol) in 1,4-dioxane (3 mL) under Ar atmosphere and stirred at 90° C. for 48 hr in closed vial. The reaction mixture was cooled and filtered. The filtercake was washed with MeOH (10 mL) and the filtrate was concentrated in vacuo to obtain N-(4-amino-1-tetrahydropyran-2-yl-pyrazolo[4,3-c]pyridin-7-yl)-N′-ethyl-N′-[1-[4-(pentafluoro-sulfanyl)phenyl]ethyl]oxamide (239 mg, crude) as a brown solid which was used in the next step without purification.

LCMS (ESI): [M+H]⁺m/z: calcd 563.19; found 563.2; Rt=1.080 min.

### Step 5: The synthesis of N-(4-amino-1H-pyrazolo[4,3-c]pyridin-7-yl)-N′-ethyl-N′-[1-[4-(pentafluoro-sulfanyl)phenyl]ethyl]oxamide

N-(4-amino-1-tetrahydropyran-2-yl-pyrazolo[4,3-c]pyridin-7-yl)-N′-ethyl-N′-[1-[4-(pentafluoro-sulfanyl)phenyl]ethyl]oxamide (239 mg, 424.85 μmol) was dissolved in Methanol (2 mL) and Dioxane/HCl (2 mL). The resulting solution was stirred at rt for 15 hr. Solvents were evaporated. Resulting crude product was purified by HPLC (0-2-9 min 18-25-80%

H₂O/MEOH/0.1% fa, flow 30 mL/min ((loading pump 4 mL MEOH/0.1% NH₄OH) target mass 478 column: Chromatorex C18 SMB100-5T 100×19 mm, 5 microM) to obtain N-(4-amino-1H-pyrazolo[4,3-c]pyridin-7-yl)-N′-ethyl-N′-[1-[4-(pentafluoro-sulfanyl)phenyl]ethyl]oxamide (22.3 mg, 42.52 μmol, 10.01% yield, HCOOH) as a light-yellow solid.

¹H NMR (500 MHz, DMSO-d₆) δ 0.89-1.11 (m, 3H), 1.30-1.72 (m, 3H), 2.98-3.04 (m, 1H), 3.57-3.63 (m, 1H), 4.97-5.64 (m, 1H), 6.62-6.83 (m, 2H), 7.39-7.63 (m, 1H), 7.63-7.72 (m, 2H), 7.75-7.96 (m, 2H), 8.17-8.23 (m, 1H), 9.48-10.61 (m, 1H), 11.69-13.57 (m, 1H).

LCMS (ESI): [M+H]⁺m/z: calcd 479.15; found 479.0; Rt=2.666 min.

### Example 152. Synthesis of N-(4-Amino-1H-pyrazolo[4,3-c]pyridin-7-yl)-N′-[[2-fluoro-4-(trifluoromethyl)phenyl]methyl]-N′-methyl-oxamide (Compound 96)

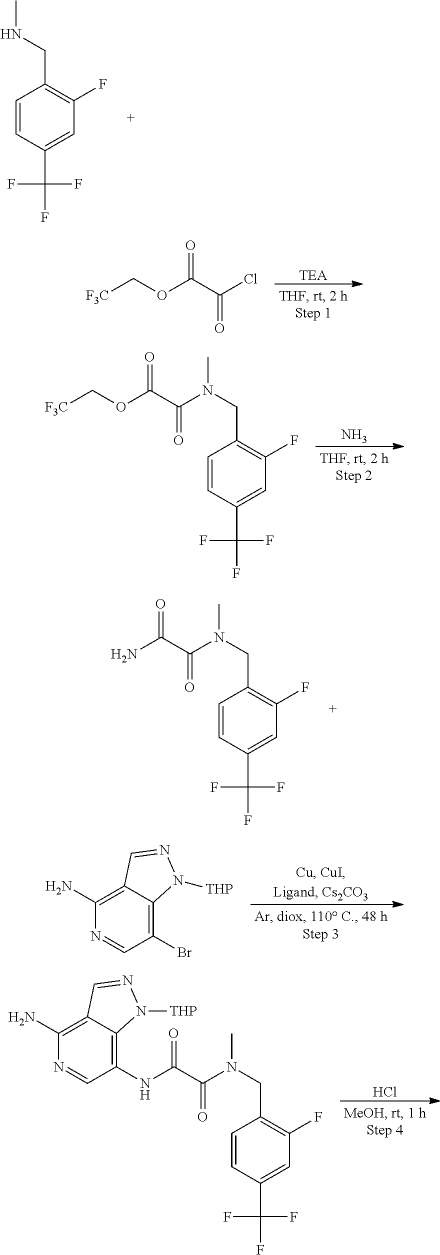

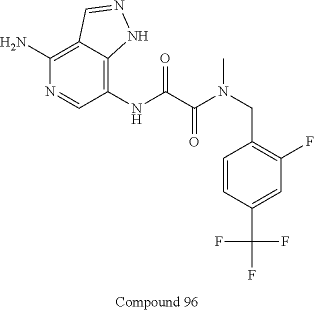 [SMILES: CN(Cc1ccc(C(F)(F)F)cc1F)C(=O)C(=O)Nc1cnc(N)c2cn[nH]c12]

### Step 1: The Synthesis of 2,2,2-Trifluoroethyl 2-[[2-fluoro-4-(trifluoromethyl)phenyl]methyl-methyl-amino]-2-oxo-acetate

2,2,2-Trifluoroethyl 2-chloro-2-oxo-acetate (404.61 mg, 2.12 mmol) was added dropwise to a stirred solution of 1-[2-fluoro-4-(trifluoromethyl)phenyl]-N-methyl-methanamine (0.4 g, 1.93 mmol) and TEA (234.45 mg, 2.32 mmol, 322.94 μL) in THF (19.80 mL) at 20° C., stirred for 1 hr at 20° C. Reaction mixture was used in the next step.

LCMS (ESI): [M+H]⁺m/z: calcd 362.0; found 362.0; Rt=1.262 min.

### Step 2: The Synthesis of N′-[[2-Fluoro-4-(trifluoromethyl)phenyl]methyl]-N′-methyl-oxamide

Ammonia (622.35 mg, 36.54 mmol) was bubbled trough a reaction mixture from previous step at 20° C., stirred for 1 hr at 20° C. Reaction mixture was filtered, solid washed with THF (2×5 mL), filtrate concentrated in vacuo to give pure N′-[[2-fluoro-4-(trifluoromethyl)phenyl]methyl]-N′-methyl-oxamide (0.3 g, 1.08 mmol, 59.02% yield).

### Step 3: The Synthesis of N-(4-Amino-2-tetrahydropyran-2-yl-pyrazolo[4,3-c]pyridin-7-yl)-N′-[2-fluoro-4-(trifluoromethyl)phenyl]methyl]-N′-methyl-oxamide

Copper (1.26 mg, 19.77 μmol), Copper (I) iodide (37.65 mg, 197.70 μmol, 6.70 μL), cesium carbonate (193.24 mg, 593.09 μmol) was added to a stirred solution of N′-[[2-fluoro-4-(trifluoromethyl)phenyl]methyl]-N′-methyl-oxamide (110.00 mg, 395.40 μmol), 7-bromo-2-tetrahydropyran-2-yl-pyrazolo[4,3-c]pyridin-4-amine (117.49 mg, 395.40 μmol), rac-(1R,2R)—N1,N2-dimethylcyclohexane-1,2-diamine (28.12 mg, 197.70 μmol) in 1,4-dioxane (7.00 mL) under Ar atmosphere and stirred at 110° C. for 48 hr in closed vial. RM was cooled to r.t., filtered, solid washed with dioxane (2×3 mL), filtrate used in the next step.

### Step 4: The Synthesis of N-(4-Amino-1H-pyrazolo[4,3-c]pyridin-7-yl)-N′-[[2-fluoro-4-(trifluoromethyl)phenyl]methyl]-N′-methyl-oxamide (Compound 96)

Hydrogen chloride (164.27 mg, 3.83 mmol, 134.65 μL, 85% purity) was added to a solution of N-(4-amino-2-tetrahydropyran-2-yl-pyrazolo[4,3-c]pyridin-7-yl)-N′-[[2-fluoro-4-(trifluoromethyl)phenyl]methyl]-N′-methyl-oxamide (0.1 g, 202.25 μmol) in Methanol (1.62 mL) and stirred at 20° C. for 1 hr. Volatiles was evaporated in vacuo and residue submitted to HPLC (column: Chromatorex 18 SMB100-5T 100×19 mm 5 μm; 5-55% 0-5 min H₂O/ACN/0.1% FA, flow rate: 30 mL/min) to give N-(4-amino-1H-pyrazolo[4,3-c]pyridin-7-yl)-N′-[2-fluoro-4-(trifluoromethyl)phenyl]methyl]-N′-methyl-oxamide (59 mg, 129.29 μmol, 63.92% yield, HCOOH).

LCMS (ESI): [M+H]⁺m/z: calcd 411.2; found 411.2; Rt=1.693 min.

### Example 153. Synthesis of N-(4-amino-1H-pyrazolo[4,3-c]pyridin-7-yl)-N′-methyl-N′—[(1R)-1-[5-(trifluoromethyl)-2-pyridyl]ethyl]oxamide (Compound 147), and N-(4-amino-1H-pyrazolo[4,3-c]pyridin-7-yl)-N′-methyl-N′—[(1S)-1-[5-(trifluoromethyl)-2-pyridyl]ethyl]oxamide (Compound 127)

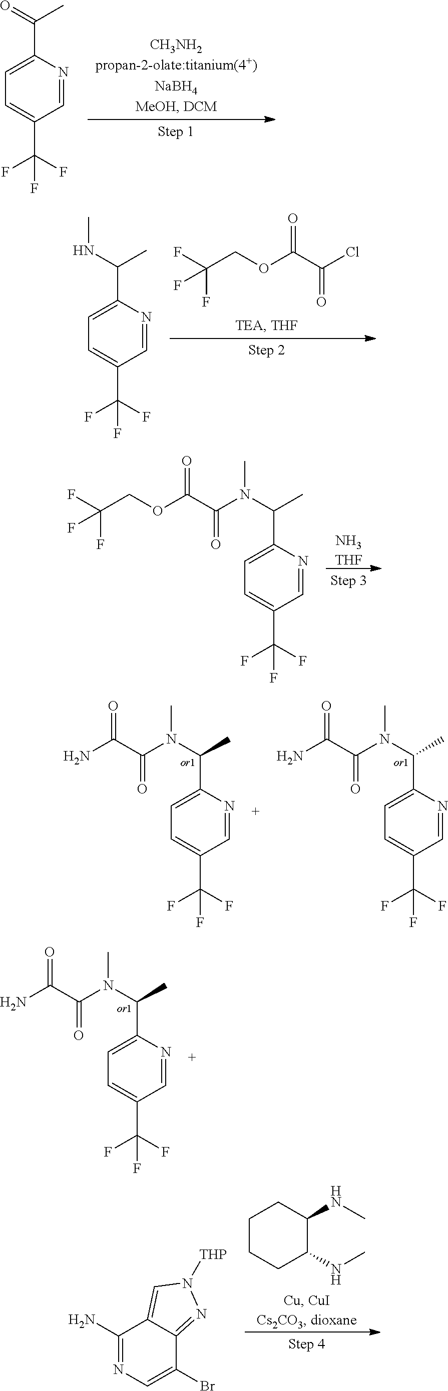

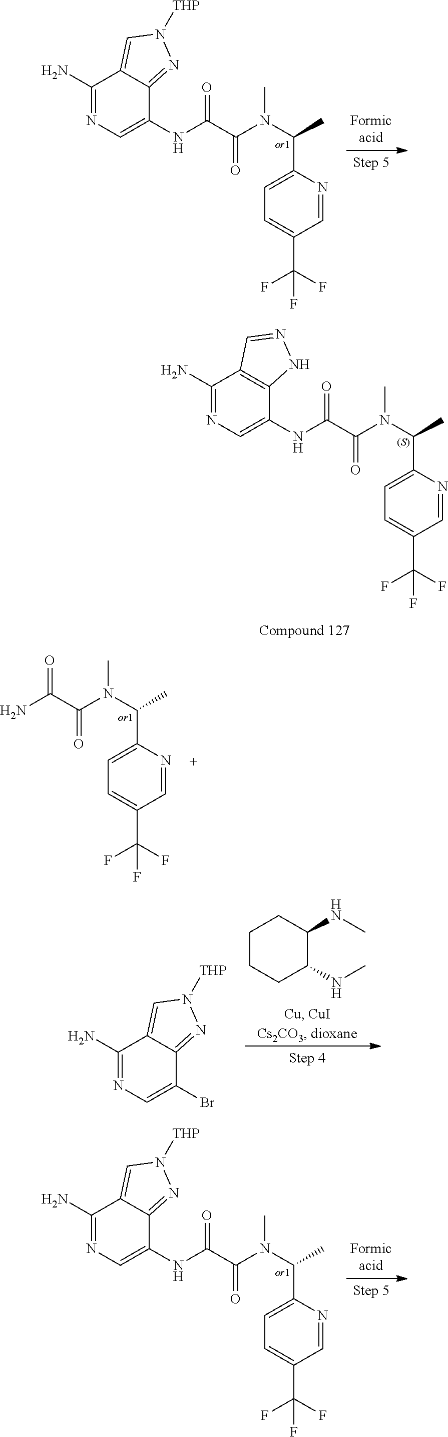

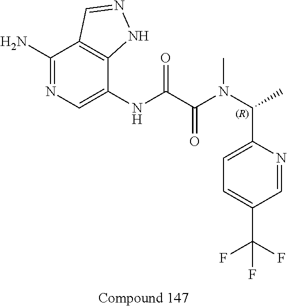

### Step 1: N-methyl-1-[5-(trifluoromethyl)-2-pyridyl]ethanamine

Propan-2-olate; titanium (4+)(1.80 g, 6.34 mmol, 1.89 mL) was added in one portion to a solution of 1-[5-(trifluoromethyl)-2-pyridyl]ethanone (1 g, 5.29 mmol), methanamine (4.93 g, 15.86 mmol, 5.48 mL) in DCM (30 mL) and the resulting mixture was stirred for 12 hr at 25° C. After 12 hr Methanol (3 mL) was added to the RM, stirred for 10 min and Sodium Borohydride (140.01 mg, 3.70 mmol, 130.36 μL) was added and stirred for 30 min. The reaction mixture was quenched with K₂CO₃ (5 mL, sat. aq.) and extracted with DCM (2×100 mL). The combined organic layer was washed with water (50 mL), dried over Na₂SO₄ and evaporated in vacuo to give N-methyl-1-[5-(trifluoromethyl)-2-pyridyl]ethanamine (0.9 g, 4.41 mmol, 83.36% yield).as a yellow oil.

LCMS (ESI): [M+H]⁺m/z: calcd 205.1; found 205.0; Rt=0.553 min.

### Step 2:2,2,2-trifluoroethyl 2-oxo-2-[1-[5-(trifluoromethyl)-2-pyridyl]ethylamino]acetate

2,2,2-trifluoroethyl 2-chloro-2-oxo-acetate (821.01 mg, 4.31 mmol) was added dropwise to a stirred solution of N-methyl-1-[5-(trifluoromethyl)-2-pyridyl]ethanamine (0.8 g, 3.92 mmol) and TEA (594.68 mg, 5.88 mmol, 819.11 μL) in THF (30 mL) at 0° C., stirred for 1 hr at 0° C. Reaction mixture was used in the next step.

LCMS (ESI): [M+H]⁺m/z: calcd 359.09; found 359.0; Rt=1.314 min.

### Step 3: N′-methyl-N′—[(1S)-1-[5-(trifluoromethyl)-2-pyridyl]ethyl]oxamide, N′-methyl-N′—[(1R)-1-[5-(trifluoromethyl)-2-pyridyl]ethyl]oxamide

Ammonia (148.43 mg, 8.72 mmol) was bubbled trough a reaction mixture at 20° C., stirred for 1 hr at 20° C. and 8 hr at 25° C. Reaction mixture was filtered, solid washed with THF (2×5 mL), filtrate concentrated in vacuo to give crude N′-[1-[5-(trifluoromethyl)-2-pyridyl]ethyl]oxamide (450 mg, 1.635 mmol, 57.10% yield). Racemic product was submitted to chiral HPLC (column: XBridge BEH C18 5 μm 130A; Oct. 10, 2045% 0-1-5 min H₂O/ACN, flow rate: 30 mL/min) to give N′-methyl-N′—[(1S)-1-[5-(trifluoromethyl)-2-pyridyl]ethyl]oxamide (0.17 g, 617.67 μmol, 16.35% yield), N′-methyl-N′—[(1R)-1-[5-(trifluoromethyl)-2-pyridyl]ethyl]oxamide (0.17 g, 617.67 μmol, 16.35% yield) as a yellow gum.

F1 RT (P1, S isomer)=11.746 min.

LCMS (ESI): [M+H]⁺m/z: calcd 276.11; found 276.0; Rt=2.160 min.

F2 RT (P2, R isomer)=19.317 min.

LCMS (ESI): [M+H]⁺m/z: calcd 276.11; found 276.0; Rt=2.160 min.

### Step 4: N-(4-amino-1-tetrahydropyran-2-yl-pyrazolo[4,3-c]pyridin-7-yl)-N′-methyl-N′—[(1S)-1-[5-(trifluoromethyl)-2-pyridyl]ethyl]oxamide

Copper (2.07 mg, 32.54 μmol), Copper (I) iodide (61.98 mg, 325.42 μmol, 11.03 μL), cesium carbonate (318.09 mg, 976.26 μmol) was added to a stirred solution of N′-methyl-N′—[(1S)-1-[5-(trifluoromethyl)-2-pyridyl]ethyl]oxamide (179.13 mg, 650.84 μmol, F1 step 3), 7-bromo-1-tetrahydropyran-2-yl-pyrazolo[4,3-c]pyridin-4-amine (203.07 mg, 683.38 μmol), rac-(1R,2R)—N1,N2-dimethylcyclohexane-1,2-diamine (46.29 mg, 325.42 μmol) in 1,4-dioxane (7.01 mL) under Ar atmosphere and stirred at 110° C. for 48 hr in closed vial. RM was filtered, solid washed with DCM (3 mL), combined filtrate used in the next step.

LCMS (ESI): [M+H]⁺m/z: calcd 492.2; found 492.0; Rt=1.147 min.

### Step 5: The synthesis of N-(4-amino-1H-pyrazolo[4,3-c]pyridin-7-yl)-N′-methyl-N′—[(1S)-1-[5-(trifluoromethyl)-2-pyridyl]ethyl]oxamide (Compound 127)

Formic acid (165.26 mg, 3.05 mmol, 135.46 μL, 85% purity) was added to a stirred solution of N-(4-amino-1-tetrahydropyran-2-yl-pyrazolo[4,3-c]pyridin-7-yl)-N′-methyl-N′—[(1S)-1-[5-(trifluoromethyl)-2-pyridyl]ethyl]oxamide (100.00 mg, 203.47 μmol, F1 step 3) in Methanol (1 mL) and stirred at 20° C. for 1 hr. Volatiles was evaporated in vacuo, the residue was submitted to HPLC (column: Chromatorex 18 SMB100-5T 100×19 mm 5 μm; 15-30% 0-5 min H₂O/ACN/0.1% NH₄OH, flow rate: 30 ml/min) to give N-(4-amino-1H-pyrazolo[4,3-c]pyridin-7-yl)-N′-methyl-N′—[(1S)-1-[5-(trifluoromethyl)-2-pyridyl]ethyl]oxamide (12 mg, 26.47 μmol, 13.01% yield, HCOOH)) as a yellow solid.

¹H NMR (600 MHz, DMSO-d₆) δ 1.60-1.68 (m, 3H), 2.73-3.03 (m, 3H), 5.69-5.83 (m, 1H), 6.69 (s, 2H), 7.57-7.73 (m, 2H), 8.18 (s, 1H), 8.24 (dd, 1H), 8.98 (d, 1H), 10.20-10.54 (m, 1H), 12.62-12.90 (m, 1H).

LCMS (ESI): [M+H]⁺m/z: calcd 408.15; found 408.2; Rt=1.903 min.

### Step 4: N-(4-amino-1-tetrahydropyran-2-yl-pyrazolo[4,3-c]pyridin-7-yl)-N′-methyl-N′—[(1R)-1-[5-(trifluoromethyl)-2-pyridyl]ethyl]oxamide

copper (2.07 mg, 32.54 μmol), Copper (I) iodide (61.98 mg, 325.42 μmol, 11.03 μL), cesium carbonate (318.09 mg, 976.26 μmol) was added to a stirred solution of N′-methyl-N′—[(1R)-1-[5-(trifluoromethyl)-2-pyridyl]ethyl]oxamide (179.13 mg, 650.84 μmol, F2 step 3), 7-bromo-1-tetrahydropyran-2-yl-pyrazolo[4,3-c]pyridin-4-amine (203.07 mg, 683.38 μmol), rac-(1R,2R)—N1,N2-dimethylcyclohexane-1,2-diamine (46.29 mg, 325.42 μmol) in 1,4-dioxane (7.01 mL) under Ar atmosphere and stirred at 110° C. for 48 hr in closed vial. RM was filtered, solid washed with Dioxane (3 mL), combined filtrate used in the next step.

LCMS (ESI): [M+H]⁺m/z: calcd 492.2; found 492.0; Rt=1.147 min.

### Step 5: The synthesis of N-(4-amino-1H-pyrazolo[4,3-c]pyridin-7-yl)-N′-methyl-N′—[(1R)-1-[5-(trifluoromethyl)-2-pyridyl]ethyl]oxamide (Compound 147)

Formic acid (165.26 mg, 3.05 mmol, 135.46 μL, 85% purity) was added to a stirred solution of N-(4-amino-1-tetrahydropyran-2-yl-pyrazolo[4,3-c]pyridin-7-yl)-N′-methyl-N′—[(1R)-1-[5-(trifluoromethyl)-2-pyridyl]ethyl]oxamide (100.00 mg, 203.47 μmol, F2 step 3) in Methanol (1 mL) and stirred at 20° C. for 1 hr. Volatiles was evaporated in vacuo, the residue was submitted to HPLC (column: XBridge C18 OBD 100×30 mm 5 μm; 0-5-20% 0-1.3-5.3 min H20/ACN/0.1% NH₄OH, flow rate: 30 ml/min) to give N-(4-amino-1H-pyrazolo[4,3-c]pyridin-7-yl)-N′-methyl-N′—[(1R)-1-[5-(trifluoromethyl)-2-pyridyl]ethyl]oxamide (12 mg, 29.46 μmol, 14.48% yield) as a yellow solid.

¹H NMR (600 MHz, DMSO-d₆) δ 1.60-1.68 (m, 3H), 2.73-3.03 (m, 3H), 5.69-5.83 (m, 1H), 6.69 (s, 2H), 7.57-7.73 (m, 2H), 8.18 (s, 1H), 8.24 (dd, 1H), 8.98 (d, 1H), 10.20-10.54 (m, 1H), 12.62-12.90 (m, 1H).

LCMS (ESI): [M+H]⁺m/z: calcd 408.15; found 408.2; Rt=1.903 min.

### Example 154. Synthesis of N-(4-Amino-2-tetrahydropyran-2-yl-pyrazolo[4,3-c]pyridin-7-yl)-N′-ethyl-N′-[[2-methyl-4-(1,1,2,2,2-pentafluoroethyl)phenyl]methyl]oxamide (Compound 99)

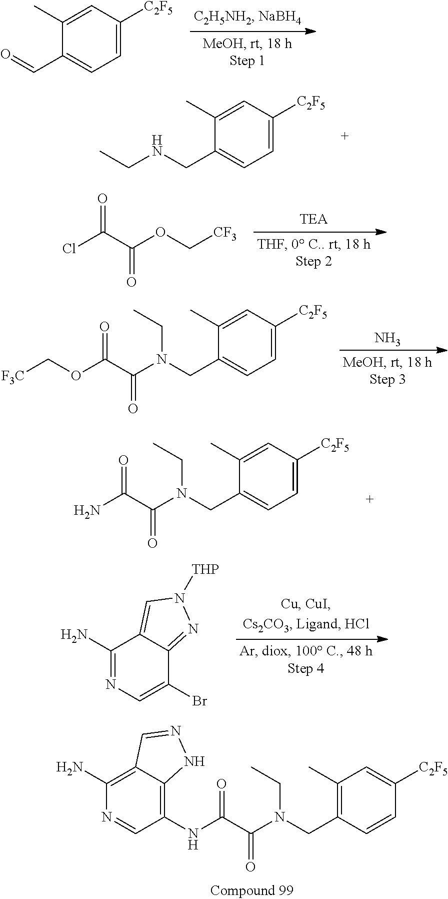

### Step 1: The Synthesis of N-[[2-Methyl-4-(1,1,2,2,2-pentafluoroethyl)phenylmethyl]ethanamine

Ethanamine (188.32 mg, 2.31 mmol, 234.52 μL, HCl) was dissolved in MeOH (3.90 mL) and Sodium acetate, anhydrous (378.89 mg, 4.62 mmol, 247.96 μL) was added. The resulting mixture was stirred for 15 min and 2-methyl-4-(1,1,2,2,2-pentafluoroethyl)benzaldehyde (500 mg, 2.10 mmol) was added. The resulting mixture was stirred for 1 hr and Sodium cyanoborohydride (197.90 mg, 3.15 mmol) was added. The resulting mixture was stirred overnight. The reaction mixture was concentrated in vacuo, and aq·K₂CO₃ (10 mL) was added. The resulting mixture was extracted with DCM (2*20 mL) and combined organic layers were dried over Na₂SO₄, filtered, and concentrated in vacuo. The residue was dissolved in MTBE (15 mL) and HCl/Et₂O (5 mL) was added. The resulting suspension was filtered. The filter cake was rinsed with MTBE and air-died to obtain N-[[2-methyl-4-(1,1,2,2,2-pentafluoroethyl)phenyl]methyl]ethanamine (458 mg, 1.51 mmol, 71.83% yield, HCl).

LCMS (ESI): [M+H]⁺m/z: calcd 268.0; found 268.0; Rt=0.874 min.

### Step 2: The Synthesis of 2,2,2-Trifluoroethyl 2-[ethyl-[[2-methyl-4-(1,1,2,2,2-pentafluoroethyl)phenyl]methyl]amino]-2-oxo-acetate

N-[[2-methyl-4-(1,1,2,2,2-pentafluoroethyl)phenyl]methyl]ethanamine (458 mg, 1.51 mmol, HCl) was suspended in DCM (10 mL) and Triethylamine (335.72 mg, 3.32 mmol, 462.43 μL) was added. The resulting mixture was cooled to −5° C. in an ice/methanol bath and 2,2,2-trifluoroethyl 2-chloro-2-oxo-acetate (301.66 mg, 1.58 mmol) was added dropwise, the resulting mixture was allowed to warm to room temperature and stirred overnight. Water (10 mL) was added and an organic layer was separated. The aqueous layer was extracted with DCM (10 mL) and combined organic layers were dried over Na₂SO₄, filtered and concentrated in vacuo to obtain 2,2,2-trifluoroethyl 2-[ethyl-[[2-methyl-4-(1,1,2,2,2-pentafluoroethyl)phenyl]methyl]amino]-2-oxo-acetate (510 mg, 1.21 mmol, 80.27% yield).

LCMS (ESI): [M+H]⁺m/z: calcd 422.1; found 422.1; Rt=1.647 min.

### Step 3: The Synthesis of N′-Ethyl-N′-[[2-methyl-4-(1,1,2,2,2-pentafluoroethyl)phenyl]methyl]oxamide

2,2,2-Trifluoroethyl 2-[ethyl-[[2-methyl-4-(1,1,2,2,2-pentafluoroethyl)phenyl]methyl]amino]-2-oxo-acetate (510 mg, 1.21 mmol) was dissolved in MeOH (10 mL) and NH3/MeOH (20 mL) was added. The resulting mixture was stirred overnight. The reaction mixture was concentrated in vacuo to obtain N′-ethyl-N′-[[2-methyl-4-(1,1,2,2,2-pentafluoroethyl)phenyl]methyl]oxamide (465 mg, crude)

LCMS (ESI): [M+H]⁺m/z: calcd 339.0; found 339.0; Rt=1.223 min.

### Step 4: The Synthesis of N-(4-Amino-2-tetrahydropyran-2-yl-pyrazolo[4,3-c]pyridin-7-yl)-N′-ethyl-N′-[[2-methyl-4-(1,1,2,2,2-pentafluoroethyl)phenyl]methyl]oxamide (Compound 99)

N′-Ethyl-N′-[2-methyl-4-(1,1,2,2,2-pentafluoroethyl)phenyl]methyl]oxamide (200 mg, 591.24 μmol), 7-bromo-2-tetrahydropyran-2-yl-pyrazolo[4,3-c]pyridin-4-amine (193.26 mg, 650.36 μmol), Copper (1.88 mg, 29.56 μmol), Copper (I) iodide (56.30 mg, 295.62 μmol, 10.02 μL), rac-(1R,2R)—N1,N2-dimethylcyclohexane-1,2-diamine (63.07 mg, 443.43 μmol) and Cesium carbonate (385.28 mg, 1.18 mmol) were mixed in Dioxane (4 mL) and the resulting mixture was purged with argon for 5 min. The reaction mixture was heated at 100° C. over the weekend. The reaction mixture was cooled and filtered. The filter cake was rinsed with MeOH (7 ml), and the filtrate was concentrated in vacuo. The residue was dissolved in MeOH (5 mL), and HCl/dioxane (5 mL) was added. The resulting mixture was stirred for 3 hr and filtered. The filtrate was concentrated in vacuo, and the residue was purified by HPLC (0-2-10 min, 38-45-60 H₂O/MeOH/0.1NH4OH, flow 30 mL/min ((loading pump 4 mL MeOH), column: XBridge BEH C18 100*19 mm, 5 microM) to obtain N-(4-amino-2-tetrahydropyran-2-yl-pyrazolo[4,3-c]pyridin-7-yl)-N′-ethyl-N′-[[2-methyl-4-(1,1,2,2,2-pentafluoroethyl)phenyl]methyl]oxamide (40.6 mg, 73.22 μmol, 12.38% yield).

¹H NMR (600 MHz, dmso) δ 1.01-1.19 (m, 3H), 2.12-2.40 (m, 3H), 3.34-3.57 (m, 2H), 4.31-4.88 (m, 2H), 6.58-7.31 (m, 2H), 7.31-7.54 (m, 3H), 7.54-7.76 (m, 1H), 8.07-8.23 (m, 1H), 9.52-10.76 (m, 1H), 12.58-13.51 (m, 1H).

LCMS (ESI): [M+H]⁺m/z: calcd 471.2; found 471.2; Rt=1.214 min.

### Example 155. Synthesis of N-(4-Amino-1H-pyrazolo[4,3-c]pyridin-7-yl)-N′-methyl-N′-[[2-methyl-4-(1,1,2,2,2-pentafluoroethyl)phenyl]methyl]oxamide (Compound 184)

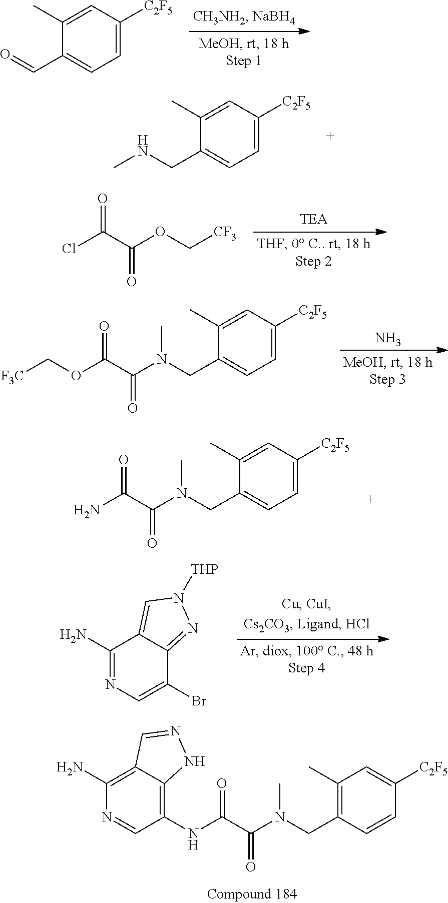

### Step 1: The Synthesis of N-Methyl-1-[2-methyl-4-(1,1,2,2,2-pentafluoroethyl)phenyl]methanamine

2-Methyl-4-(1,1,2,2,2-pentafluoroethyl)benzaldehyde (416 mg, 1.75 mmol) was dissolved in MeOH (4 mL) and methanamine (352.62 mg, 2.27 mmol, 449.20 μL) was added thereto. The resulting mixture was stirred for 2 hr and Sodium Borohydride (198.24 mg, 5.24 mmol, 184.58 μL) was added portionwise. The resulting mixture was stirred overnight. The reaction mixture was concentrated in vacuo and water (10 mL) was added. The resulting mixture was extracted with DCM (2*20 mL) and combined organic layers were dried over Na₂SO₄, filtered and concentrated in vacuo to obtain N-methyl-1-[2-methyl-4-(1,1,2,2,2-pentafluoroethyl)phenyl]methanamine (385 mg, 1.52 mmol, 87.04% yield).

LCMS (ESI): [M+H]⁺m/z: calcd 254.0; found 254.0; Rt=0.867 min.

### Step 2: The Synthesis of 2,2,2-Trifluoroethyl 2-[methyl-[[2-methyl-4-(1,1,2,2,2-pentafluoroethyl)phenyl]methyl]amino]-2-oxo-acetate

N-Methyl-1-[2-methyl-4-(1,1,2,2,2-pentafluoroethyl)phenyl]methanamine (455 mg, 1.80 mmol) and Triethylamine (200.01 mg, 1.98 mmol, 275.50 μL) were dissolved in DCM (10 mL) and the resulting solution was cooled to −5° C. in an ice/methanol bath. 2,2,2-trifluoroethyl 2-chloro-2-oxo-acetate (359.44 mg, 1.89 mmol) was added dropwise and the resulting mixture was allowed to warm to room temperature and stirred overnight. Water (10 mL) was added and an organic layer was separated. The aqueous layer was extracted with DCM (10 mL) and combined organic layers were dried over Na₂SO₄, filtered and concentrated in vacuo to obtain 2,2,2-trifluoroethyl 2-[methyl-[2-methyl-4-(1,1,2,2,2-pentafluoroethyl)phenyl]methyl]amino]-2-oxo-acetate (610 mg, 1.50 mmol, 83.36% yield)

LCMS (ESI): [M+H]⁺m/z: calcd 408.0; found 408.0; Rt=1.605 min.

### Step 3: The Synthesis of N′-Methyl-N′-[[2-methyl-4-(1,1,2,2,2-pentafluoroethyl)phenylmethyl]oxamide

2,2,2-Trifluoroethyl 2-[methyl-[[2-methyl-4-(1,1,2,2,2-pentafluoroethyl)phenyl]methyl]amino]-2-oxo-acetate (610 mg, 1.50 mmol) was dissolved in MeOH (10 mL) and NH3/MeOH (20 mL) was added. The resulting mixture was stirred overnight. The reaction mixture was concentrated in vacuo to obtain N′-methyl-N′-[[2-methyl-4-(1,1,2,2,2-pentafluoroethyl)phenyl]methyl]oxamide (484 mg, 1.49 mmol, 99.66% yield).

LCMS (ESI): [M+H]⁺m/z: calcd 325.1; found 325.1; Rt=1.062 min.

### Step 4: The Synthesis of N-(4-Amino-1H-pyrazolo[4,3-c]pyridin-7-yl)-N′-methyl-N′-[[2-methyl-4-(1,1,2,2,2-pentafluoroethyl)phenyl]methyl]oxamide (Compound 184)

N′-Methyl-N′-[2-methyl-4-(1,1,2,2,2-pentafluoroethyl)phenyl]methyl]oxamide (200 mg, 616.82 μmol), 7-bromo-2-tetrahydropyran-2-yl-pyrazolo[4,3-c]pyridin-4-amine (201.62 mg, 678.50 μmol), Copper (1.96 mg, 30.84 μmol), Copper (I) iodide (58.74 mg, 308.41 μmol, 10.45 μL), rac-(1R,2R)—N1,N2-dimethylcyclohexane-1,2-diamine (65.80 mg, 462.61 μmol) and Cesium carbonate (401.94 mg, 1.23 mmol) were mixed in Dioxane (4 mL). The resulting mixture was purged with argon for 5 min and the resulting mixture was heated at 100° C. over the weekend. The reaction mixture was cooled and filtered. The filter cake was rinsed with MeOH (7 mL) and the filtrate was concentrated in vacuo. The residue was dissolved in MeOH (5 mL) and HCl/dioxane (5 mL) was added. The resulting mixture was stirred for 3 hr and filtered. The filtrate was concentrated in vacuo and the residue was purified by HPLC (0-2-10 min, 33-40-65 H₂O/MeOH/0.1NH4OH, flow 30 mL/min ((loading pump 4 mL MeOH), column: XBridge BEH C18 100*19 mm, 5 microM) to obtain N-(4-amino-1H-pyrazolo[4,3-c]pyridin-7-yl)-N′-methyl-N′-[2-methyl-4-(1,1,2,2,2-pentafluoroethyl)phenyl]methyl]oxamide (118.1 mg, 258.78 μmol, 41.95% yield).

¹H NMR (600 MHz, dmso) 8 2.15-2.40 (m, 3H), 2.85-3.14 (m, 3H), 4.64-4.94 (m, 2H), 6.60-6.95 (m, 2H), 7.35-7.42 (m, 1H), 7.47-7.57 (m, 2H), 7.58-7.72 (m, 1H), 8.12-8.20 (m, 1H), 9.71-10.51 (m, 1H), 12.66-13.38 (m, 1H).

LCMS (ESI): [M+H]⁺m/z: calcd 457.0; found 457.0; Rt=0.938 min.

### Example 156. Synthesis of N-(4-Amino-1H-pyrazolo[4,3-c]pyridin-7-yl)-N′-ethyl-N′-[[2-fluoro-4-(1,1,2,2,2-pentafluoroethyl)phenyl]methyl]oxamide (Compound 157)

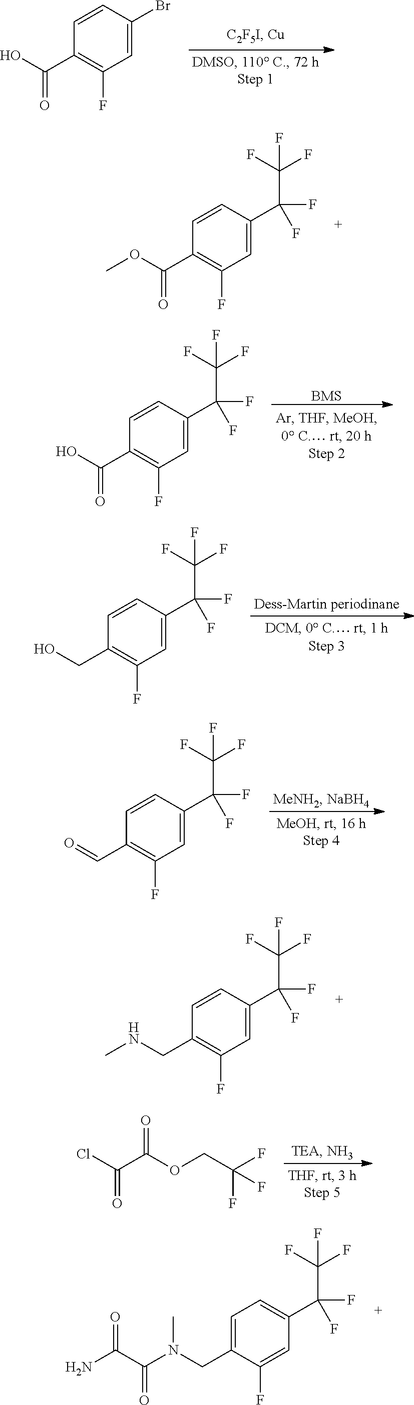

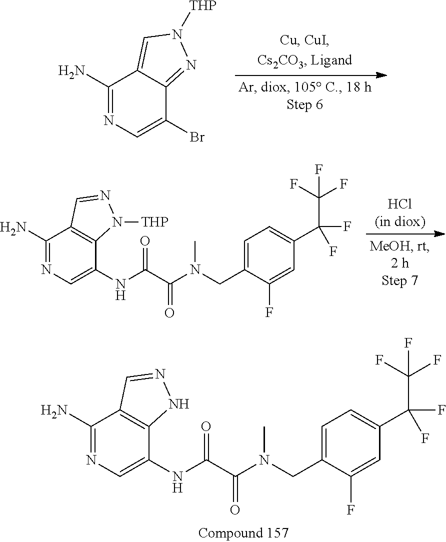

Steps 1-3 were described for Compound 189

### Step 4: The Synthesis of 1-[2-Fluoro-4-(1,1,2,2,2-pentafluoroethyl)phenyl]-N-methyl-methanamine

2-Fluoro-4-(1,1,2,2,2-pentafluoroethyl)benzaldehyde (0.7 g, 2.89 mmol) was added to a solution of methanamine (6.73 g, 43.37 mmol, 7.49 mL) in methanol (15 mL) at 25° C. The resulting mixture was stirred at 25° C. for 15 hr, then cooled to 0° C. and Sodium Borohydride (218.74 mg, 5.78 mmol, 203.67 μL) was added in one portion. The reaction mixture was allowed to warm to 25° C. and stirred for 1 hr, then concentrated in vacuo. The residue was diluted with water (25 mL) and extracted with dichloromethane (40 mL). The organic layer was separated, dried over sodium sulfate and concentrated in vacuo to afford 1-[2-fluoro-4-(1,1,2,2,2-pentafluoroethyl)phenyl]-N-methyl-methanamine (0.3 g, 1.17 mmol, 40.35% yield) as yellow oil.

LCMS (ESI): [M+H]⁺m/z: calcd 258.2; found 258.2; Rt=0.776 min.

### Step 5: The Synthesis of N′-[[2-Fluoro-4-(1,1,2,2,2-pentafluoroethyl)phenyl]methyl]-N′-methyl-oxamide

2,2,2-Trifluoroethyl 2-chloro-2-oxo-acetate (346.92 mg, 1.82 mmol) was added slowly to a cooled to −10° C. mixture of 1-[2-fluoro-4-(1,1,2,2,2-pentafluoroethyl)phenyl]-N-methyl-methanamine (0.3 g, 1.17 mmol) and triethyl amine (578.19 mg, 5.71 mmol, 796.41 μL) in THF (51.97 mL). The resulting mixture was allowed to warm to 25° C. and stirred for 2 hr. Then, gaseous ammonia (19.87 mg, 1.17 mmol, 23.10 μL) was vigorously bubbled through it at 25° C. for 1 hr. The resulting mixture was filtered to remove ammonium chloride, the filtercake was washed with THF (2*20 mL) and discarded. The combined filtrate was concentrated in vacuo to afford crude N′-[[2-fluoro-4-(1,1,2,2,2-pentafluoroethyl)phenyl]methyl]-N′-methyl-oxamide (0.36 g, 1.10 mmol, 94.03% yield) as red gum, which was used directly in the next step.

LCMS (ESI): [M+H]⁺m/z: calcd 329.2; found 329.0; Rt=1.007 min.

### Step 6: The Synthesis of N-(4-Amino-2-tetrahydropyran-2-yl-pyrazolo[4,3-c]pyridin-7-yl)-N′-[2-fluoro-4-(1,1,2,2,2-pentafluoroethyl)phenyl]methyl]-N′-methyl-oxamide

A mixture of N′-[[2-fluoro-4-(1,1,2,2,2-pentafluoroethyl)phenyl]methyl]-N′-methyl-oxamide (360 mg, 1.10 mmol), 7-bromo-2-tetrahydropyran-2-yl-pyrazolo[4,3-c]pyridin-4-amine (300 mg, 1.01 mmol), copper (5.85 mg, 92.03 μmol), Copper (I) iodide (120 mg, 630.09 μmol, 21.35 μL), cesium carbonate (500.33 mg, 1.54 mmol) and rac-(1R,2R)—N1,N2-dimethylcyclohexane-1,2-diamine (120 mg, 843.64 μmol) in 1,4-dioxane (6 mL) was stirred in a sealed vial under argon at 105° C. for 18 hr. The resulting mixture was cooled down and filtered. The filtercake was washed successively with THF (2*5 mL) and dichloromethane (3*5 mL). The combined filtrate was concentrated in vacuo to afford crude N-(4-amino-2-tetrahydropyran-2-yl-pyrazolo[4,3-c]pyridin-7-yl)-N′-[2-fluoro-4-(1,1,2,2,2-pentafluoroethyl)phenyl]methyl]-N′-methyl-oxamide (0.9 g, crude) as brown gum, which was used directly in the next step.

LCMS (ESI): [M+H]⁺m/z: calcd 545.0; found 545.0; Rt=2.832 min.

### Step 7: The Synthesis of N-(4-Amino-1H-pyrazolo[4,3-c]pyridin-7-yl)-N′-ethyl-N′-[[2-fluoro-4-(1,1,2,2,2-pentafluoroethyl)phenyl]methyl]oxamide (Compound 157)

Hydrogen chloride solution 4.0M in dioxane (5.25 g, 20.02 mmol, 5 mL, 13.9% purity) was added to a stirred solution of crude from previous step N-(4-amino-2-tetrahydropyran-2-yl-pyrazolo[4,3-c]pyridin-7-yl)-N′-ethyl-N′-[[2-fluoro-4-(1,1,2,2,2-pentafluoroethyl)phenyl]methyl]oxamide (0.9 g, 1.61 mmol) in methanol (5 mL) at 25° C. The resulting solution was stirred at 25° C. for 2 hr, then concentrated to dryness in vacuo and the residue was submitted to reverse phase HPLC (column: Chromatorex 18 SNB100-5T 100*19 mm 5 μm; mobile phase: Oct. 10, 2045% 0-1-6 min H₂O/MeCN/0.1% FA; flow: 30 mL/min (loading pump 4 mL/min acetonitrile)) to afford N-(4-amino-1H-pyrazolo[4,3-c]pyridin-7-yl)-N′-ethyl-N′-[2-fluoro-4-(1,1,2,2,2-pentafluoroethyl)phenyl]methyl]oxamide (122 mg, 234.44 μmol, 14.55% yield, HCOOH) as light-yellow solid.

¹H NMR (600 MHz, dmso) & 2.86-3.22 (m, 3H), 4.36-5.06 (m, 2H), 6.02-7.35 (m, 2H), 7.45-7.77 (m, 4H), 8.15-8.39 (m, 1H), 9.73-10.63 (m, 1H), 12.66-13.53 (m, 1H).

LCMS (ESI): [M+H]⁺m/z: calcd 461.2; found 461.2; Rt=2.419 min.

### Example 157. Synthesis of N-(4-Amino-1H-pyrazolo[4,3-c]pyridin-7-yl)-N′-[[2-fluoro-4-(trifluoromethyl)phenyl]methyl]-N′-methyl-oxamide (Compound 137)

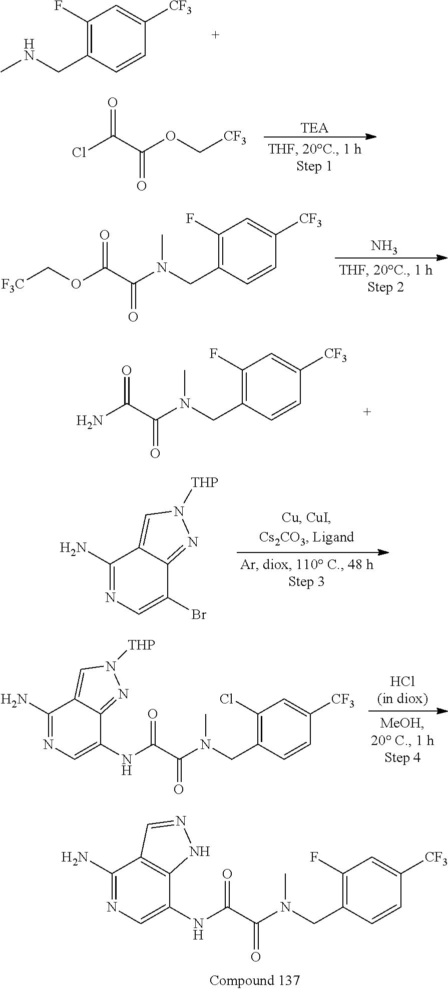

### Step 1: The Synthesis of 2,2,2-Trifluoroethyl 2-[[2-fluoro-4-(trifluoromethyl)phenyl methyl-methyl-amino]-2-oxo-acetate

2,2,2-Trifluoroethyl 2-chloro-2-oxo-acetate (404.61 mg, 2.12 mmol) was added dropwise to a stirred solution of 1-[2-fluoro-4-(trifluoromethyl)phenyl]-N-methyl-methanamine (0.4 g, 1.93 mmol) and TEA (234.45 mg, 2.32 mmol, 322.94 μL) in THF (19.80 mL) at 20° C., stirred for 1 hr at 20° C. Reaction mixture was used in the next step.

LCMS (ESI): [M+H]⁺m/z: calcd 362.0; found 362.0; Rt=1.262 min.

### Step 2: The Synthesis of N′-[[2-Fluoro-4-(trifluoromethyl)phenyl]methyl]-N′-methyl-oxamide

Ammonia (622.35 mg, 36.54 mmol) was bubbled trough a reaction mixture from previous step at 20° C., stirred for 1 hr at 20° C. Reaction mixture was filtered, solid washed with THF (2×5 mL), filtrate concentrated in vacuo to give pure N′-[[2-fluoro-4-(trifluoromethyl)phenyl]methyl]-N′-methyl-oxamide (0.3 g, 1.08 mmol, 59.02% yield).

LCMS (ESI): [M+H]⁺m/z: calcd 279.2; found 279.2; Rt=1.055 min.

### Step 3: The Synthesis of N-(4-Amino-2-tetrahydropyran-2-yl-pyrazolo[4,3-c]pyridin-7-yl)-N′-[[2-chloro-4-(trifluoromethyl)phenyl]methyl]-N′-methyl-oxamide

Copper (1.29 mg, 20.36 μmol), Copper (I) iodide (38.78 mg, 203.63 μmol, 6.90 μL), cesium carbonate (199.04 mg, 610.88 μmol) was added to a stirred solution of N′-[[2-chloro-4-(trifluoromethyl)phenyl]methyl]-N′-methyl-oxamide (0.12 g, 407.25 μmol), 7-bromo-2-tetrahydropyran-2-yl-pyrazolo[4,3-c]pyridin-4-amine (121.02 mg, 407.25 μmol), rac-(1R,2R)—N1,N2-dimethylcyclohexane-1,2-diamine (28.96 mg, 203.63 μmol) in 1,4-dioxane (7.00 mL) under Ar atmosphere and stirred at 110° C. for 48 hr in closed vial. RM was cooled to r.t., filtered, solid washed with dioxane (2×3 mL), filtrate used in the next step.

LCMS (ESI): [M+H]⁺m/z: calcd 511.0; found 511.0; Rt=1.040 min.

### Step 4: The Synthesis of N-(4-Amino-1H-pyrazolo[4,3-c]pyridin-7-yl)-N′-[[2-fluoro-4-(trifluoromethyl)phenyl]methyl]-N′-methyl-oxamide (Compound 137)

Hydrogen chloride solution 4.0M in dioxane (147.48 mg, 4.04 mmol) was added to a solution of N-(4-amino-2-tetrahydropyran-2-yl-pyrazolo[4,3-c]pyridin-7-yl)-N′-[[2-fluoro-4-(trifluoromethyl)phenyl]methyl]-N′-methyl-oxamide (0.2 g, 404.50 μmol) in Methanol (1.49 mL) and stirred at 20° C. for 1 hr. Volatiles was evaporated in vacuo and residue submitted to HPLC (column: Chromatorex 18 SMB100-5T 100×19 mm 5 μm; May 5, 2045% 0-1-5 min H₂O/ACN/0.1% FA, flow: 30 mL/min, flow rate: 30 mL/min) to give N-(4-amino-1H-pyrazolo[4,3-c]pyridin-7-yl)-N′-[[2-fluoro-4-(trifluoromethyl)phenyl]methyl]-N′-methyl-oxamide (56 mg, 122.71 μmol, 30.34% yield, HCOOH).

¹H NMR (600 MHz, dmso) 8 2.86-3.24 (m, 3H), 4.38-5.02 (m, 2H), 6.62-7.06 (m, 2H), 7.30-7.58 (m, 1H), 7.58-7.72 (m, 1H), 7.72-7.80 (m, 1H), 7.85-7.96 (m, 1H), 8.11-8.24 (m, 2H), 9.68-10.60 (m, 1H), 12.65-13.44 (m, 1H).

LCMS (ESI): [M+H]⁺m/z: calcd 427.0; found 427.0; Rt=2.287 min.

### Example 158. Synthesis of N-(4-Amino-1H-pyrazolo[4,3-c]pyridin-7-yl)-N′-ethyl-N′-[2-fluoro-4-(1,1,2,2,2-pentafluoroethyl)phenyl]methyl]oxamide (Compound 189)

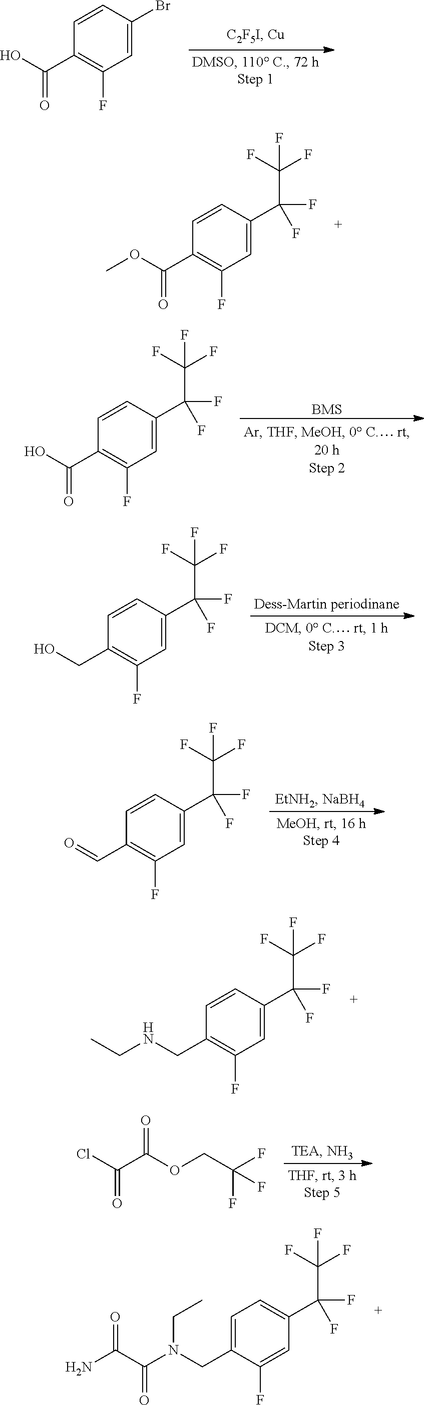

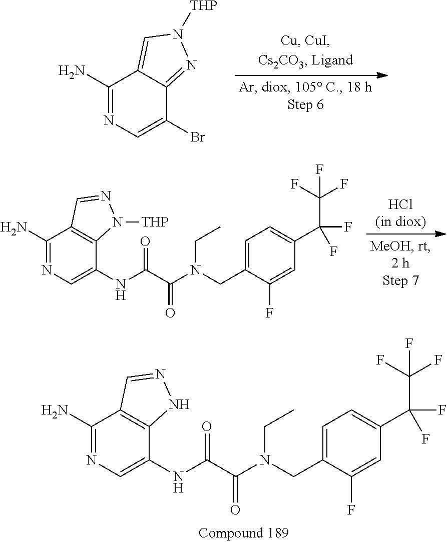

### Step 1: The Synthesis of Methyl 2-fluoro-4-(1,1,2,2,2-pentafluoroethyl)benzoate and 2-Fluoro-4-(1,1,2,2,2-pentafluoroethyl)benzoic acid

4-Bromo-2-fluoro-benzoic acid (10 g, 45.66 mmol) and Copper (29.02 g, 456.60 mmol) were added in one portion at 25° C. to a solution of 1,1,1,2,2-pentafluoro-2-iodo-ethane (78.60 g, 319.62 mmol) in DMSO(200 mL). The resulting mixture was stirred in autoclave at (gradually warmed during 2-3 hr) 110° C. for 72 hr, then cooled down and poured in water (800 mL) and MTBE\EtOAc mixture (1\1, 600 mL)(CAUTION: huge evolution of gaseous products!). The resulting slurry was stirred for 15 min, and filtered. The filtercake was washed with EtOAc (5* 100 mL) and discarded. The filtrate was transferred to a separatory funnel, the organic layer was separated, washed with water (3*100 mL), dried over sodium sulfate and concentrated in vacuo to afford crude product, which was purified by column chromatography on silica gel using chloroform\acetonitrile gradient (0-100% acetonitrile) to afford methyl 2-fluoro-4-(1,1,2,2,2-pentafluoroethyl)benzoate (1.5 g, 5.51 mmol, 12.07% yield) as yellow oil and Cu-salt of the target acid (5.5 g) as green powder. It was dissolved in a mixture of dichloromethane (100 mL) and 6N aqueous hydrochloric acid (50 mL), the organic layer was separated, dried over sodium sulfate and concentrated in vacuo to afford 2-fluoro-4-(1,1,2,2,2-pentafluoroethyl)benzoic acid (4.8 g, 18.60 mmol, 40.73% yield) as light-yellow solid.

LCMS (ESI): [M+H]⁺m/z: calcd 259.0; found 259.0; Rt=1.295 min.

### Step 2: The Synthesis of [2-Fluoro-4-(1,1,2,2,2-pentafluoroethyl)phenyl]methanol

Borane dimethyl sulfide complex (3.11 g, 40.91 mmol, 3.88 mL) was added dropwise under argon to a cooled to 0° C. solution of 2-fluoro-4-(1,1,2,2,2-pentafluoroethyl)benzoic acid (4.8 g, 18.60 mmol) in THF (99.43 mL). The resulting mixture was allowed to warm to 25° C. and stirred for 18 hr. The reaction mixture was again cooled to 0° C. and methanol (50 mL) was added dropwise over 0.5 hr. The resulting mixture was allowed to warm to 25° C. and stirred for 2 hr. The reaction mixture was concentrated in vacuo, the residue was diluted with water (40 mL) and extracted with MTBE (2*80 mL). The combined organic extracts were dried over sodium sulfate and concentrated in vacuo to afford crude [2-fluoro-4-(1,1,2,2,2-pentafluoroethyl)phenyl]methanol (4.5 g, 18.43 mmol, 99.12% yield) as yellow oil, which was used directly in the next step.

¹H NMR (500 MHz, CDCl₃) δ (ppm) 4.85 (s, 2H), 7.29 (m, 1H), 7.44 (m, 1H), 7.62 (m, 1H)

### Step 3: The Synthesis of 2-Fluoro-4-(1,1,2,2,2-pentafluoroethyl)benzaldehyde

1,1-Bis(acetyloxy)-3-oxo-3H-1 lambda 5,2-benziodaoxol-1-yl acetate (9.38 g, 22.12 mmol) was added in one portion to a cooled to 0° C. solution of [2-fluoro-4-(1,1,2,2,2-pentafluoroethyl)phenyl]methanol (4.5 g, 18.43 mmol) in dichloromethane (100 mL). The resulting mixture was allowed to warm to 25° C. over 1 hr period. The reaction mixture was carefully neutralized with 5% aqueous sodium hydrogen carbonate solution. The resulting slurry was filtered, the filtercake was washed with dichloromethane (2*20 mL) and discarded. The combined filtrate was transferred to a separatory funnel, the dichloromethane layer was separated, dried over sodium sulfate and concentrated in vacuo. The residue was triturated with hexane (100 mL), stirred for 10 min, and then filtered. The filtercake was additionally washed with hexane (2*20 mL) and then discarded. The combined hexane filtrate was concentrated in vacuo to afford 2-fluoro-4-(1,1,2,2,2-pentafluoroethyl)benzaldehyde (3.5 g, 14.46 mmol, 78.43% yield) as yellow oil.

¹H NMR (500 MHz, CDCl₃) δ (ppm) 7.54 (m, 2H), 8.04 (m, 1H), 10.44 (m, 1H).

### Step 4: The Synthesis of N-[[2-Fluoro-4-(1,1,2,2,2-pentafluoroethyl)phenyl methyl]ethanamine

2-Fluoro-4-(1,1,2,2,2-pentafluoroethyl)benzaldehyde (0.7 g, 2.89 mmol) was added to a solution of ethanamine (931.02 mg, 14.46 mmol, 1.16 mL) in methanol (19.07 mL) at 25° C. The resulting mixture was stirred at 25° C. for 15 hr, then cooled to 0° C. and Sodium Borohydride (218.74 mg, 5.78 mmol, 203.67 μL) was added in one portion. The reaction mixture was allowed to warm to 25° C. and stirred for 1 hr, then concentrated in vacuo. The residue was diluted with water (25 mL) and extracted with dichloromethane (40 mL). The organic layer was separated, dried over sodium sulfate and concentrated in vacuo to afford N-[[2-fluoro-4-(1,1,2,2,2-pentafluoroethyl)phenyl]methyl]ethanamine (0.7 g, 2.58 mmol, 89.28% yield) as yellow oil.

LCMS (ESI): [M+H]⁺m/z: calcd 272.2; found 272.2; Rt=0.798 min.

### Step 5: The Synthesis of N′-Ethyl-N′-[[2-fluoro-4-(1,1,2,2,2-pentafluoroethyl)phenyl]methyl]oxamide

2,2,2-Trifluoroethyl 2-chloro-2-oxo-acetate (548.29 mg, 2.88 mmol) was added slowly to a cooled to −10° C. mixture of N-[[2-fluoro-4-(1, 1,2,2,2-pentafluoroethyl)phenyl]methyl]ethanamine (500 mg, 1.84 mmol) and triethyl amine (913.82 mg, 9.03 mmol, 1.26 mL) in THF (51.49 mL). The resulting mixture was allowed to warm to 25° C. and stirred for 2 hr. Then, gaseous ammonia (31.40 mg, 1.84 mmol) was vigorously bubbled through it at 25° C. for 1 hr. The resulting mixture was filtered to remove ammonium chloride, the filtercake was washed with THF (2*20 mL) and discarded. The combined filtrate was concentrated in vacuo to afford crude N′-ethyl-N′-[2-fluoro-4-(1,1,2,2,2-pentafluoroethyl)phenyl]methyl]oxamide (600 mg, 1.75 mmol, 95.09% yield) as red gum, which was used directly in the next step.

LCMS (ESI): [M+H]⁺m/z: calcd 343.2; found 343.0; Rt=1.327 min.

### Step 6: The Synthesis of N-(4-Amino-2-tetrahydropyran-2-yl-pyrazolo[4,3-c]pyridin-7-yl)-N′-ethyl-N′-[[2-fluoro-4-(1,1,2,2,2-pentafluoroethyl)phenyl]methyl]oxamide

A mixture of N′-ethyl-N′-[[2-fluoro-4-(1,1,2,2,2-pentafluoroethyl)phenyl]methyl]oxamide (300 mg, 876.59 μmol), 7-bromo-2-tetrahydropyran-2-yl-pyrazolo[4,3-c]pyridin-4-amine (300 mg, 1.01 mmol), copper (4.67 mg, 73.55 μmol), Copper (I) iodide (121.52 mg, 638.07 μmol, 21.62 μL), cesium carbonate (428.41 mg, 1.31 mmol) and rac-(1R,2R)—N1,N2-dimethylcyclohexane-1,2-diamine (121.52 mg, 854.34 μmol) in 1,4-dioxane (6 mL) was stirred in a sealed vial under argon at 105° C. for 18 hr. The resulting mixture was cooled down and filtered. The filtercake was washed successively with THF (2*5 mL) and dichloromethane (3*5 mL). The combined filtrate was concentrated in vacuo to afford crude N-(4-amino-2-tetrahydropyran-2-yl-pyrazolo[4,3-c]pyridin-7-yl)-N′-ethyl-N′-[2-fluoro-4-(1,1,2,2,2-pentafluoroethyl)phenyl]methyl]oxamide (0.9 g, crude) as brown gum, which was used directly in the next step.

LCMS (ESI): [M+H]⁺m/z: calcd 559.0; found 559.0; Rt=2.963 min.

### Step 7: The Synthesis of N-(4-Amino-1H-pyrazolo[4,3-c]pyridin-7-yl)-N′-ethyl-N′-[[2-fluoro-4-(1,1,2,2,2-pentafluoroethyl)phenyl]methyl]oxamide (Compound 189)

Hydrogen chloride solution 4.0M in dioxane (5.25 g, 20.02 mmol, 5 mL, 13.9% purity) was added to a stirred solution of crude from previous step N-(4-amino-2-tetrahydropyran-2-yl-pyrazolo[4,3-c]pyridin-7-yl)-N′-ethyl-N′-[[2-fluoro-4-(1,1,2,2,2-pentafluoroethyl)phenyl]methyl]oxamide (0.9 g, 1.61 mmol) in methanol (5 mL) at 25° C. The resulting solution was stirred at 25° C. for 2 hr, then concentrated to dryness in vacuo to afford 800 mg of crude product, 100 mg of which was purified by reverse phase HPLC (column: XBridge BEH C18 5 μm 130 A; mobile phase: 35-40-90% 0-1.3-5.3 min H₂O/MeOH/0.1% NH₄OH; flow: 30 mL/min; (loading pump 4 mL/min MeOH)) to afford Compound 189-N-(4-amino-1H-pyrazolo[4,3-c]pyridin-7-yl)-N′-ethyl-N′-[2-fluoro-4-(1,1,2,2,2-pentafluoroethyl)phenyl]methyl]oxamide (8.7 mg, 18.34 μmol, 1.14% yield) as grey solid. The remaining 700 mg of crude product was then purified by reverse phase HPLC (column: Chromatorex 18 SNB100-5T 100*19 mm 5 μm; mobile phase: Oct. 15, 2045% 0-1.3-5.3 min H₂O/MeCN/0.1% FA; flow: 30 mL/min (loading pump 4 mL/min MeCN)) to afford a second batch of Compound 189-N-(4-amino-1H-pyrazolo[4,3-c]pyridin-7-yl)-N′-ethyl-N′-[[2-fluoro-4-(1, 1,2,2,2-pentafluoroethyl)phenyl]methyl]oxamide (69 mg, 132.59 μmol, 8.23% yield, HCOOH) as light-brown solid.

LCMS (ESI): [M+H]⁺m/z: calcd 475.0; found 475.0; Rt=2.586 min.

### Example 159. Synthesis of N-(4-Amino-1H-pyrazolo[4,3-c]pyridin-7-yl)-N′-ethyl-N′—[(1S)-1-[2-fluoro-4-(trifluoromethyl)phenyl]ethyl]oxamide (Compound 153) and N-(4-Amino-1H-pyrazolo[4,3-c]pyridin-7-yl)-N′-ethyl-N′—[(1R)-1-[2-fluoro-4-(trifluoromethyl)phenyl]ethyl]oxamide (Compound 126)

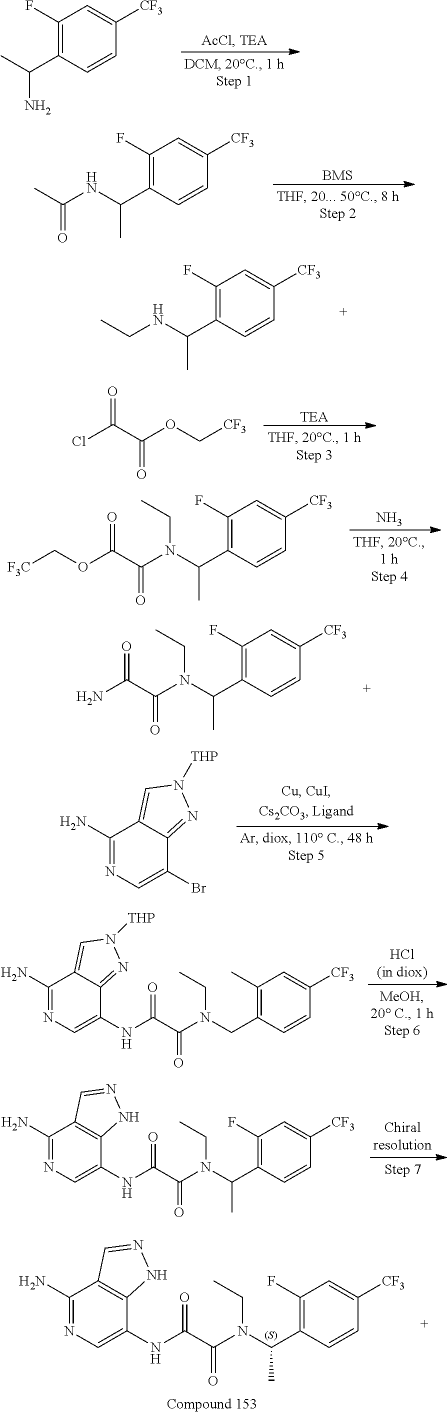

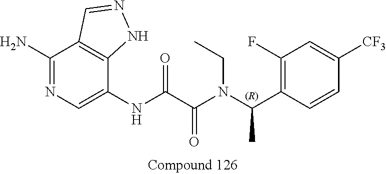

### Step 1: The Synthesis of N-[1-[2-Fluoro-4-(trifluoromethyl)phenyl]ethyl]acetamide

Acetyl chloride (458.48 mg, 5.84 mmol, 354.31 μL) was added dropwise to a stirred solution of 1-[2-fluoro-4-(trifluoromethyl)phenyl]ethanamine (1.1 g, 5.31 mmol) and TEA (698.48 mg, 6.90 mmol, 962.09 μL) in DCM (18.68 mL) at 20° C., stirred for 1 hr at 20° C. Reaction mixture washed with water (2×10 mL), DCM dried over sodium sulfate, concentrated in vacuo to give N-[1-[2-fluoro-4-(trifluoromethyl)phenyl]ethyl]acetamide (1.1 g, 4.41 mmol, 83.13% yield).

LCMS (ESI): [M+H]⁺m/z: calcd 250.0; found 250.0; Rt=1.142 min.

### Step 2: The Synthesis of N-Ethyl-1-[2-fluoro-4-(trifluoromethyl)phenyl]ethanamine

Borane dimethyl sulfide complex (1.01 g, 13.24 mmol, 1.26 mL) was added to a stirred solution of N-[1-[2-fluoro-4-(trifluoromethyl)phenyl]ethyl]acetamide (1.1 g, 4.41 mmol) in THF (20 mL) at 20° C. and stirred at 50° C. for 8 hr. Methanol (3 mL) was added dropwise and volatiles was evaporated in vacuo to give crude N-ethyl-1-[2-fluoro-4-(trifluoromethyl)phenyl]ethanamine (0.8 g, 3.40 mmol, 77.05% yield).

LCMS (ESI): [M+H]⁺m/z: calcd 236.2; found 236.2; Rt=0.718 min.

### Step 3: The Synthesis of 2,2,2-Trifluoroethyl 2-[ethyl-[1-[2-fluoro-4-(trifluoromethyl)phenyl]ethyl]amino]-2-oxo-acetate

2,2,2-Trifluoroethyl 2-chloro-2-oxo-acetate (213.81 mg, 1.12 mmol) was added dropwise to a stirred solution of N-ethyl-1-[2-fluoro-4-(trifluoromethyl)phenyl]ethanamine (0.24 g, 1.02 mmol) and TEA (123.90 mg, 1.22 mmol, 170.65 μL) in THF (15 mL) at 20° C., stirred for 1 hr at 20° C. Reaction mixture was used in the next step.

LCMS (ESI): [M+H]⁺m/z: calcd 390.0; found 390.0; Rt=1.318 min.

### Step 4: The Synthesis of N′-Ethyl-N′-[1-[2-fluoro-4-(trifluoromethyl)phenyl]ethyl]oxamide

Ammonia (341.25 mg, 20.04 mmol) was bubbled trough a reaction mixture from previous step at 20° C., stirred for 1 hr at 20° C. Reaction mixture was filtered, solid washed with THF (2×5 mL), filtrate concentrated in vacuo to give pure N′-ethyl-N′-[1-[2-fluoro-4-(trifluoromethyl)phenyl]ethyl]oxamide (0.2 g, 653.05 μmol, 65.18% yield).

### Step 5: The Synthesis of N-(4-Amino-2-tetrahydropyran-2-yl-pyrazolo[4,3-c]pyridin-7-yl)-N′-ethyl-N′-[1-[2-fluoro-4-(trifluoromethyl)phenyl]ethyl]oxamide

Copper (1.56 mg, 24.49 μmol), Copper (I) iodide (46.64 mg, 244.89 μmol, 8.30 μL), cesium carbonate (239.37 mg, 734.68 μmol) was added to a stirred solution of N′-ethyl-N′-[1-[2-fluoro-4-(trifluoromethyl)phenyl]ethyl]oxamide (0,15 g, 489.79 μmol), 7-bromo-2-tetrahydropyran-2-yl-pyrazolo[4,3-c]pyridin-4-amine (145.54 mg, 489.79 μmol), rac-(1R,2R)—N1,N2-dimethylcyclohexane-1,2-diamine (34.83 mg, 244.89 μmol) in 1,4-dioxane (7.00 mL) under Ar atmosphere and stirred at 110° C. for 48 hr in closed vial. RM was cooled to r.t., filtered, solid washed with dioxane (2×3 mL), filtrate used in the next step.

### Step 6: The Synthesis of N-(4-Amino-1H-pyrazolo[4,3-c]pyridin-7-yl)-N′-ethyl-N′-[1-[2-fluoro-4-(trifluoromethyl)phenyl]ethyl]oxamide

Hydrogen chloride (388.61 mg, 9.06 mmol, 318.54 μL, 85% purity) was added to a solution of N-(4-amino-2-tetrahydropyran-2-yl-pyrazolo[4,3-c]pyridin-7-yl)-N′-ethyl-N′-[1-[2-fluoro-4-(trifluoromethyl)phenyl]ethyl]oxamide (0.25 g, 478.47 μmol) in Methanol (2 mL) and stirred at 20° C. for 1 hr. Volatiles was evaporated in vacuo and residue submitted to HPLC (column: XBridge C18 100×19 mm, 5 μm; 30-80% 0-5 min H₂O/MeOH/0.1% NH₄OH, flow rate: 30 ml/min) to give N-(4-amino-1H-pyrazolo[4,3-c]pyridin-7-yl)-N′-ethyl-N′-[1-[2-fluoro-4-(trifluoromethyl)phenyl]ethyl]oxamide (18 mg, 41.06 μmol, 8.58% yield). Pure racemic product was submitted to chiral HPLC.

LCMS (ESI): [M+H]⁺m/z: calcd 439.2; found 439.2; Rt=2.320 min.

### Step 7: The Synthesis of N-(4-Amino-1H-pyrazolo[4,3-c]pyridin-7-yl)-N′-ethyl-N′—[(1S)-1-[2-fluoro-4-(trifluoromethyl)phenyl]ethyl]oxamide (Compound 153) and N-(4-Amino-1H-pyrazolo[4,3-c]pyridin-7-yl)-N′-ethyl-N′—[(1R)-1-[2-fluoro-4-(trifluoromethyl)phenyl]ethyl]oxamide (Compound 126)

Racemic N-(4-amino-1H-pyrazolo[4,3-c]pyridin-7-yl)-N′-ethyl-N′-[1-[2-fluoro-4-(trifluoromethyl)phenyl]ethyl]oxamide (18 mg, 41.06 μmol) was separated into enantiomers by chiral HPLC (Column: CHIRALPAK IC (250×30 mm, 10 mkm)-II; Injection Volume: 500mkl; Mobile Phase: Hexane: IPA: MeOH, 50:25:25; Flow Rate: 28 ml/min) to give Compound 153-N-(4-amino-1H-pyrazolo[4,3-c]pyridin-7-yl)-N′-ethyl-N′—[(1S)-1-[2-fluoro-4-(trifluoromethyl)phenyl]ethyl]oxamide (8.2 mg, 18.71 μmol, 45.56% yield) F1 (RT=8.818 min) and Compound 126-N-(4-amino-1H-pyrazolo[4,3-c]pyridin-7-yl)-N′-ethyl-N′—[(1R)-1-[2-fluoro-4-(trifluoromethyl)phenyl]ethyl]oxamide (8.7 mg, 19.85 μmol, 48.33% yield) F2 (RT=12.014 min).

The absolute stereochemistry of the two compounds was independently confirmed.

### Compound 153:

Yield: 8.2 mg (45.56%)

RT (Chiralpak IC (250×4.6 mm, 5 mkm)-1; Hexane (0.1% EDA): IPA: MeOH, 50:25:25; Flow Rate: 0.6 mL/min)=8.818 min.

¹H NMR (600 MHz, dmso) 8 0.80-1.00 (m, 3H), 1.57-1.72 (m, 3H), 3.08-3.25 (m, 1H), 3.41-3.53 (m, 1H), 5.60-5.96 (m, 1H), 6.59-6.83 (m, 2H), 7.58-7.84 (m, 4H), 8.10-8.22 (m, 1H), 9.48-10.61 (m, 1H), 12.49-13.41 (m, 1H).

LCMS (ESI): [M+H]⁺m/z: calcd 439.2; found 439.0; Rt=2.373 min.

### Compound 126:

Yield: 8.7 mg (48.33%)

RT (Chiralpak IC (250×4.6 mm, 5 mkm)-1; Hexane (0.1% EDA): IPA: MeOH, 50:25:25; Flow Rate: 0.6 mL/min)=12.014 min.

¹H NMR (600 MHz, dmso) 8 0.81-1.03 (m, 3H), 1.24-1.70 (m, 3H), 3.09-3.24 (m, 1H), 3.42-3.52 (m, 1H), 5.55-6.01 (m, 1H), 6.65 (s, 2H), 7.39-7.75 (m, 3H), 7.77-7.84 (m, 1H), 8.09-8.20 (m, 1H), 9.51-10.50 (m, 1H), 12.55-13.26 (m, 1H).

LCMS (ESI): [M+H]⁺m/z: calcd 439.2; found 439.0; Rt=2.376 min.

### Example 160. Synthesis of N-(4-Amino-1H-pyrazolo[4,3-c]pyridin-7-yl)-N′-ethyl-N′-[[2-methyl-4-(trifluoromethyl)phenyl]methyl]oxamide (Compound 104)

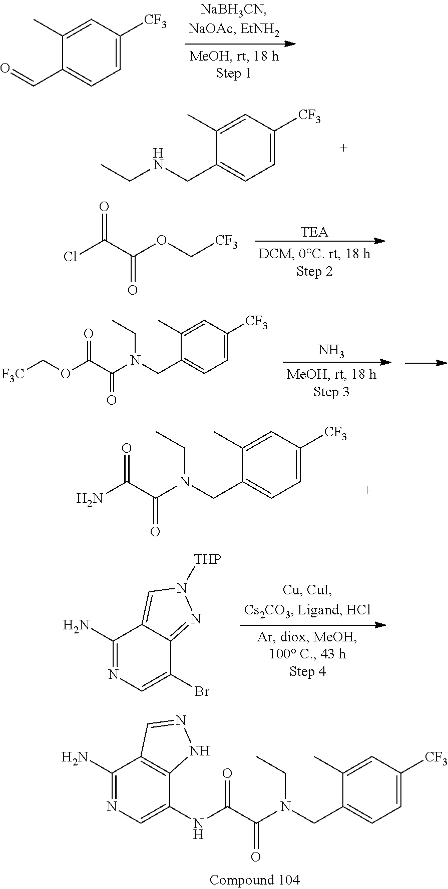

### Step 1: The Synthesis of N-[2-Methyl-4-(trifluoromethyl)phenyl]methyl]ethanamine

Ethanamine (238.38 mg, 2.92 mmol, 296.86 μL, HCl) was dissolved in MeOH (5 mL) and Sodium acetate, anhydrous (457.81 mg, 5.58 mmol, 299.61 μL) was added. The resulting mixture was stirred for 15 min and 2-methyl-4-(trifluoromethyl)benzaldehyde (500 mg, 2.66 mmol) was added. The resulting mixture was stirred for 1.5 hr and Sodium cyanoborohydride (250.50 mg, 3.99 mmol) was added. The resulting mixture was stirred overnight. The reaction mixture was concentrated in vacuo, and aq·K₂CO₃ (10 mL) was added to the residue. The resulting mixture was extracted with DCM (2*15 mL). Combined organic layers were dried over Na₂SO₄, filtered, and concentrated in vacuo. The residue was dissolved in DCM (10 mL) and HCl/Et₂O (5 mL) was added. The resulting mixture was concentrated in vacuo and the residue was triturated with MTBE, filtered, and air-dried to obtain N-[[2-methyl-4-(trifluoromethyl)phenyl]methyl]ethanamine (387 mg, 1.53 mmol, 57.40% yield, HCl).

LCMS (ESI): [M+H]⁺m/z: calcd 218.2; found 218.2; Rt=0.629 min.

### Step 2: The Synthesis of 2,2,2-Trifluoroethyl 2-[ethyl-[[2-methyl-4-(trifluoromethyl)phenyl]methyl]amino]-2-oxo-acetate

N-[2-Methyl-4-(trifluoromethyl)phenyl]methyl]ethanamine (387 mg, 1.53 mmol, HCl) was suspended in DCM (10 mL) and Triethylamine (355.03 mg, 3.51 mmol, 489.70 μL) was added. The resulting mixture was stirred for 10 min and then cooled to 0° C. in an ice bath. A solution of 2,2,2-trifluoroethyl 2-chloro-2-oxo-acetate (305.14 mg, 1.60 mmol) in DCM (5 mL) was added dropwise and the resulting mixture was allowed to warm to room temperature and stirred overnight. Water (20 mL) was added to the reaction mixture and an organic layer was separated. The aqueous layer was extracted with DCM (15 mL) and combined organic layers were dried over Na₂SO₄, filtered and concentrated in vacuo to obtain 2,2,2-trifluoroethyl 2-[ethyl-[[2-methyl-4-(trifluoromethyl)phenyl]methyl]amino]-2-oxo-acetate (502 mg, 1.35 mmol, 88.63% yield).

LCMS (ESI): [M+H]⁺m/z: calcd 372.0; found 372.0; Rt=1.484 min.

### Step 3: The Synthesis of N′-Ethyl-N′-[[2-methyl-4-(trifluoromethyl)phenylmethyl]oxamide

2,2,2-Trifluoroethyl 2-[ethyl-[[2-methyl-4-(trifluoromethyl)phenyl]methyl]amino]-2-oxo-acetate (502 mg, 1.35 mmol) was dissolved in MeOH (10 mL) and NH3/MeOH (15 mL) was added. The resulting mixture was stirred overnight. The reaction mixture was concentrated in vacuo to obtain N′-ethyl-N′-[2-methyl-4-(trifluoromethyl)phenyl]methyl]oxamide (407 mg, crude).

LCMS (ESI): [M+H]⁺m/z: calcd 289.2; found 289.2; Rt=1.241 min.

### Step 4: The Synthesis of N-(4-Amino-1H-pyrazolo[4,3-c]pyridin-7-yl)-N′-ethyl-N′-[[2-methyl-4-(trifluoromethyl)phenyl]methyl]oxamide (Compound 104)

To an 8 mL vial N′-ethyl-N′-[[2-methyl-4-(trifluoromethyl)phenyl]methyl]oxamide (150 mg, 520.35 μmol), 7-bromo-2-tetrahydropyran-2-yl-pyrazolo[4,3-c]pyridin-4-amine (170.09 mg, 572.39 μmol), Copper (1.65 mg, 26.02 μmol), Copper (I) iodide (49.55 mg, 260.18 μmol, 8.82 μL), rac-(1R,2R)—N1,N2-dimethylcyclohexane-1,2-diamine (55.51 mg, 390.27 μmol), Cesium carbonate (339.08 mg, 1.04 mmol) and Dioxane (4 mL) were charged. The resulting mixture was purged with argon for 5 min and the vial was capped and heated at 100° C. for 40 hr. The reaction mixture was cooled to room temperature and filtered. The filter cake was rinsed with MeOH (5 mL) and the filtrate was concentrated in vacuo. The residue was re-dissolved in MeOH (5 mL) and HCl/dioxane (5 mL) was added thereto. The resulting mixture was stirred for 3 hr. The reaction mixture was concentrated in vacuo and the residue was purified by HPLC (0-2-9 min, 13-20-40% MeCN/water+NH₄OH, 30 mL/min (loading pump 4 mL/min MeCN), target MI 421, column: XBridge BEH C18 100*19 mm, 5 microM) to obtain N-(4-amino-1H-pyrazolo[4,3-c]pyridin-7-yl)-N′-ethyl-N′-[2-methyl-4-(trifluoromethyl)phenyl]methyl]oxamide (87.6 mg, 208.38 μmol, 40.05% yield).

¹H NMR (500 MHz, dmso) 8 0.93-1.17 (m, 3H), 2.22-2.41 (m, 3H), 3.46-3.50 (m, 2H), 4.24-4.94 (m, 2H), 6.47-7.04 (m, 2H), 7.08-7.78 (m, 4H), 8.08-8.58 (m, 1H).

LCMS (ESI): [M+H]⁺m/z: calcd 421.0; found 421.0; Rt=1.094 min.

### Example 161. Synthesis of N-(4-Amino-1H-pyrazolo[4,3-c]pyridin-7-yl)-N′-methyl-N′-[[2-methyl-4-(trifluoromethyl)phenyl]methyl]oxamide (Compound 170)

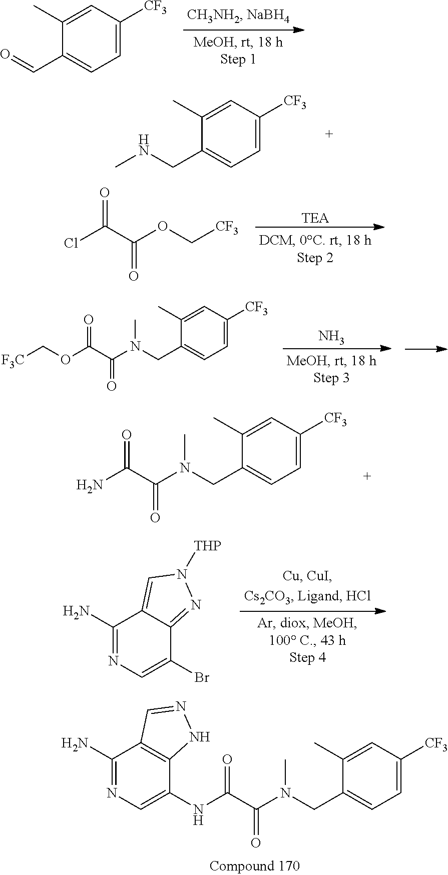

### Step 1: The Synthesis of N-methyl-1-[2-methyl-4-(trifluoromethyl)phenyl]methanamine

2-Methyl-4-(trifluoromethyl)benzaldehyde (500 mg, 2.66 mmol) was dissolved in MeOH (5 mL) and methanamine (536.47 mg, 3.45 mmol, 683.40 μL) was added. The resulting mixture was stirred for 1.5 hr and Sodium Borohydride (301.62 mg, 7.97 mmol, 280.84 μL) was added. The resulting mixture was stirred overnight. The reaction mixture was concentrated in vacuo, and water (10 mL) was added to the residue. The resulting mixture was extracted with DCM (2*15 mL). Combined organic layers were dried over Na₂SO₄, filtered, and concentrated in vacuo to obtain N-methyl-1-[2-methyl-4-(trifluoromethyl)phenyl]methanamine (480 mg, 2.36 mmol, 88.89% yield).

¹H NMR (500 MHz, CDCl₃) δ (ppm) 2.41 (s, 3H), 2.52 (s, 3H), 3.79 (s, 2H), 7.44 (m, 3H).

### Step 2: The Synthesis of 2,2,2-Trifluoroethyl 2-[methyl-[[2-methyl-4-(trifluoromethyl)phenyl]methyl]amino]-2-oxo-acetate

N-Methyl-1-[2-methyl-4-(trifluoromethyl)phenyl]methanamine (480 mg, 2.36 mmol) and Triethylamine (262.93 mg, 2.60 mmol, 362.66 μL) were dissolved in DCM (10 mL) and the resulting solution was cooled to 0° C. in an ice bath. A solution of 2,2,2-trifluoroethyl 2-chloro-2-oxo-acetate (472.50 mg, 2.48 mmol) in DCM (5 mL) was added dropwise and the resulting mixture was allowed to warm to room temperature and stirred overnight. Water (20 mL) was added to the reaction mixture and an organic layer was separated. The aqueous layer was extracted with DCM (15 mL) and combined organic layers were dried over Na₂SO₄, filtered and concentrated in vacuo to obtain 2,2,2-trifluoroethyl 2-[methyl-[[2-methyl-4-(trifluoromethyl)phenyl]methyl]amino]-2-oxo-acetate (750 mg, 2.10 mmol, 88.88% yield).

LCMS (ESI): [M+H]⁺m/z: calcd 358.0; found 358.0; Rt=1.434 min.

### Step 3: The Synthesis of N′-Methyl-N′-[[2-methyl-4-(trifluoromethyl)phenyl]methyl]oxamide

2,2,2-Trifluoroethyl 2-[methyl-[2-methyl-4-(trifluoromethyl)phenyl]methyl]amino]-2-oxo-acetate (750 mg, 2.10 mmol) was dissolved in MeOH (10 mL) and NH3/MeOH (15 mL) was added. The resulting solution was stirred overnight. The reaction mixture was concentrated in vacuo to obtain N′-methyl-N′-[2-methyl-4-(trifluoromethyl)phenyl]methyl]oxamide (590 mg, crude).

LCMS (ESI): [M+H]⁺m/z: calcd 275.0; found 275.0; Rt=1.178 min.

### Step 4: The Synthesis of N-(4-Amino-1H-pyrazolo[4,3-c]pyridin-7-yl)-N′-methyl-N′-[[2-methyl-4-(trifluoromethyl)phenyl]methyl]oxamide (Compound 170)

To an 8 mL vial N′-methyl-N′-[2-methyl-4-(trifluoromethyl)phenyl]methyl]oxamide (150 mg, 546.97 μmol), 7-bromo-2-tetrahydropyran-2-yl-pyrazolo[4,3-c]pyridin-4-amine (178.79 mg, 601.67 μmol), Copper (1.74 mg, 27.35 μmol), Copper (I) iodide (52.09 mg, 273.48 μmol, 9.27 μL), rac-(1R,2R)—N1,N2-dimethylcyclohexane-1,2-diamine (58.35 mg, 410.23 μmol), Cesium carbonate (356.43 mg, 1.09 mmol) and Dioxane (4 mL) were charged. The resulting mixture was purged with argon for 5 min and the vial was capped and heated at 100° C. for 40 hr. The reaction mixture was cooled to room temperature and filtered. The filter cake was rinsed with MeOH (5 mL) and the filtrate was concentrated in vacuo. The residue was re-dissolved in MeOH (5 mL) and HCl/dioxane (5 mL) was added thereto. The resulting mixture was stirred for 3 hr. The reaction mixture was concentrated in vacuo and the residue was purified by HPLC (0-2-9 min, Aug. 15, 2030% MeCN/H₂O+NH₄OH, flow 30 mL/min (loading pump 4 mL/min MeCN), column: XBridge BEH C18 100*19 mm, 5 microM) to obtain N-(4-amino-1H-pyrazolo[4,3-c]pyridin-7-yl)-N′-methyl-N′-[2-methyl-4-(trifluoromethyl)phenyl]methyl]oxamide (65.4 mg, 160.94 μmol, 29.42% yield).

¹H NMR (500 MHz, DMSO-d₆) δ 2.11-2.39 (m, 3H), 2.80-3.13 (m, 3H), 4.29-4.93 (m, 2H), 6.59-7.03 (m, 2H), 7.16-7.39 (m, 1H), 7.41-7.70 (m, 3H), 8.05-8.22 (m, 1H), 9.70-13.65 (m, 1H).

LCMS (ESI): [M+H]⁺m/z: calcd 407.0; found 407.0; Rt=1.050 min.

### Example 162. Synthesis of(S)-N1-(4-amino-1H-pyrazolo[4,3-c]pyridin-7-yl)-N2-(1-(2-chloro-4-(trifluoromethyl)phenyl)ethyl)-N2-methyloxalamide (Compound 167) and (R)—N¹-(4-amino-1H-pyrazolo[4,3-c]pyridin-7-yl)-N2-(1-(2-chloro-4-(trifluoromethyl)phenyl)ethyl)-N2-methyloxalamide (Compound 187)

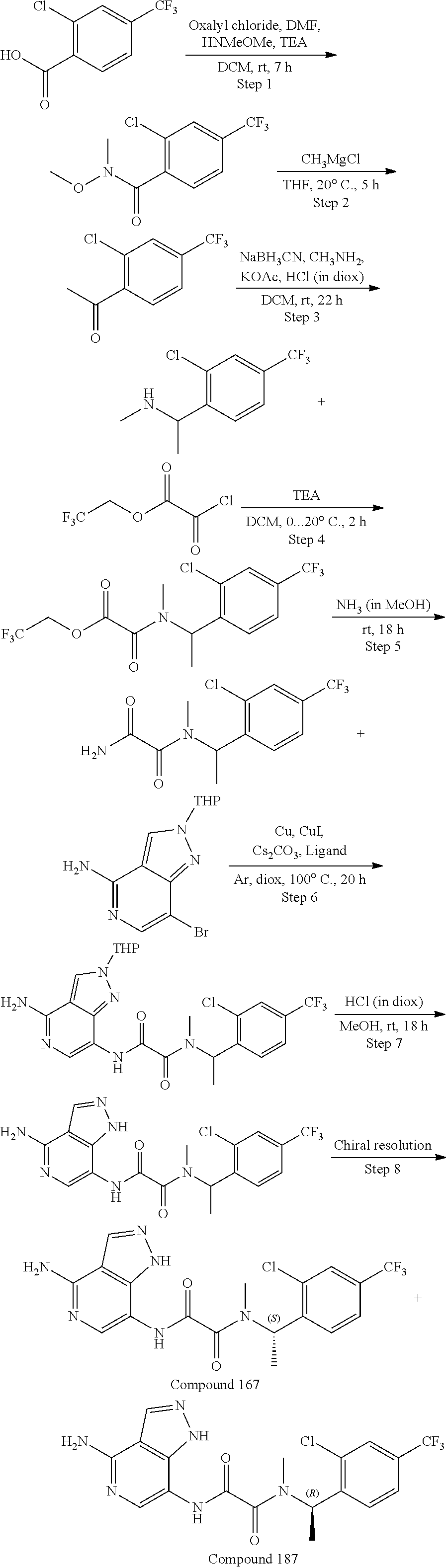

### Step 1: The Synthesis of 2-Chloro-N-methoxy-N-methyl-4-(trifluoromethyl)benzamide

Oxalyl chloride (1.70 g, 13.36 mmol, 1.17 mL) was added to the solution of 2-chloro-4-(trifluoromethyl)benzoic acid (2 g, 8.91 mmol) in Dichloromethane (30 mL) followed by Dimethylformamide (32.55 mg, 445.31 μmol, 34.48 μL). Resulting reaction mixture was stirred at 25° C. for 2.5 hr. After gas evolution ceased and solution became clear volatiles were removed under reduced pressure and residue was redissolved in Dichloromethane (10 mL). This solution was added dropwise to the suspension of N,O-Dimethylhydroxylamine hydrochloride (1.13 g, 11.58 mmol) and Triethylamine (2.70 g, 26.72 mmol, 3.72 mL) in Dichloromethane (40 mL). Resulting reaction mixture was stirred at 25° C. for 4 hr. Then, K₂CO₃ soln. (30 mL, 20% aq. soln.) was added and stirring was continued for 10 minutes. Then, organic layer was separated, dried over solid K₂CO₃ and concentrated under reduced pressure, leaving 2-chloro-N-methoxy-N-methyl-4-(trifluoromethyl)benzamide (2.28 g, 8.52 mmol, 95.65% yield).

LCMS (ESI): [M+H]⁺m/z: calcd 268.2; found 268.0; Rt=1.176 min.

### Step 2: The Synthesis of 1-[2-Chloro-4-(trifluoromethyl)phenyl]ethenone

Methylmagnesium chloride, 3M in THF (4.34 g, 12.78 mmol, 4.30 mL, 22% purity) was added dropwise to the solution of 2-chloro-N-methoxy-N-methyl-4-(trifluoromethyl)benzamide (2.28 g, 8.52 mmol) in Tetrahydrofuran (40 mL). Resulting reaction mixture was stirred at 20° C. for 5 hr. Then, it was quenched with NH₄Cl (30 mL, saturated aqueous solution) and stirring was continued for 10 minutes. Organic layer was separated, dried over solid K₂CO₃ and concentrated under reduced pressure, leaving 1-[2-chloro-4-(trifluoromethyl)phenyl]ethanone (1.8 g, 8.09 mmol, 94.92% yield).

¹H NMR (500 MHz, CDCl₃) δ (ppm) 2.65 (s, 3H), 7.58-7.60 (m, 2H), 7.68 (s, 1H).

### Step 3: The Synthesis of 1-[2-Chloro-4-(trifluoromethyl)phenyl]-N-methyl-ethanamine

To the stirred suspension of Methylamine hydrochloride (5.46 g, 80.87 mmol) and Potassium Acetate (7.94 g, 80.87 mmol, 5.05 mL) in Methanol (34.16 mL) was added 1-[2-chloro-4-(trifluoromethyl)phenyl]ethanone (1.8 g, 8.09 mmol) followed by Sodium cyanoborohydride (762.26 mg, 12.13 mmol). Resulting reaction mixture was stirred at 25° C. for 20 hr. Then, solvent was removed under reduced pressure and residue was partitioned between MTBE (40 mL) and K₂CO₃ (20% aqueous solution)(50 mL). Organic layer was separated, dried over solid K₂CO₃ and filtered. Then, filtrate was acidified with Hydrogen chloride solution 4.0M in dioxane (5.90 g, 16.17 mmol, 5.84 mL, 10% purity) and resulting solution was leaved for 2 hours for crystallization. Resulting crystalline precipitate was filtered and dried, affording 1-[2-chloro-4-(trifluoromethyl)phenyl]-N-methyl-ethanamine (1.07 g, 3.90 mmol, 48.27% yield, HCl).

¹H NMR (500 MHz, CDCl₃) δ (ppm) 1.59 (d, 3H), 4.71 (q, 1H), 7.90 (d, 1H), 8.00 (s, 1H), 8.16 (d, 1H), 9.75 (br s, 1H), 10.17 (br s, 1H).

### Step 4: The Synthesis of 2,2,2-Trifluoroethyl 2-[1-[2-chloro-4-(trifluoromethyl)phenyl]ethyl-methyl-amino]-2-oxo-acetate

2,2,2-Trifluoroethyl 2-chloro-2-oxo-acetate (816.27 mg, 4.28 mmol) was added dropwise to the solution of 1-[2-chloro-4-(trifluoromethyl)phenyl]-N-methyl-ethanamine (870 mg, 3.17 mmol, HCl) and Triethylamine (642.33 mg, 6.35 mmol, 884.75 μL) in Dichloromethane (20 mL) at 0° C. After addition was complete, cooling bath was removed and resulting mixture was allowed to warm up to 20° C. and stirred for 2 hr. Then, NaHCO₃ (20 mL, 10% aqueous solution) was added and stirring was continued for 5 min. After that, organic layer was separated, dried over Na₂SO₄ and concentrated under reduced pressure, affording 2,2,2-trifluoroethyl 2-[1-[2-chloro-4-(trifluoromethyl)phenyl]ethyl-methyl-amino]-2-oxo-acetate (1.26 g, crude).

LCMS (ESI): [M+H]⁺m/z: calcd 392.2; found 392.0; Rt=1.284 min.

### Step 5: The Synthesis of N′-[1-[2-Chloro-4-(trifluoromethyl)phenyl]ethyl]-N′-methyl-oxamide

2,2,2-Trifluoroethyl 2-[1-[2-chloro-4-(trifluoromethyl)phenyl]ethyl-methyl-amino]-2-oxo-acetate (1.26 g, 3.22 mmol) was dissolved in Ammonia (7N in methanol, 15,3% w/w)(19.48 g, 174.96 mmol, 25 mL, 15.3% purity). Resulting reaction mixture was stirred at 25° C. for 18 hr. Then, it was evaporated to dryness under reduced pressure, leaving N′-[1-[2-chloro-4-(trifluoromethyl)phenyl]ethyl]-N′-methyl-oxamide (1.04 g, crude).

### Step 6: The Synthesis of N-(4-Amino-2-tetrahydropyran-2-yl-pyrazolo[4,3-c]pyridin-7-yl)-N′-[1-[2-chloro-4-(trifluoromethyl)phenyl]ethyl]-N′-methyl-oxamide

N′-[1-[2-Chloro-4-(trifluoromethyl)phenyl]ethyl]-N′-methyl-oxamide (1.04 g, 3.37 mmol), 7-bromo-2-tetrahydropyran-2-yl-pyrazolo[4,3-c]pyridin-4-amine (1.15 g, 3.87 mmol), Copper (21.41 mg, 336.91 μmol), Copper (I) iodide (320.83 mg, 1.68 mmol, 57.09 μL), (S,S)-(+)—N,N′-Dimethyl-1,2-cyclohexanediamine (239.62 mg, 1.68 mmol) and Cesium carbonate (1.65 g, 5.05 mmol) were mixed together in Dioxane (25 mL). Reaction flask was purged with argon and resulting mixture was stirred at 100° C. for 20 hr under inert atmosphere. Then, it was diluted with DCM (30 mL) and filtered. Filtrate was concentrated under reduced pressure, affording N-(4-amino-2-tetrahydropyran-2-yl-pyrazolo[4,3-c]pyridin-7-yl)-N′-[1-[2-chloro-4-(trifluoromethyl)phenyl]ethyl]-N′-methyl-oxamide (2.5 g, crude).

LCMS (ESI): [M+H]⁺m/z: calcd 525.2; found 525.2; Rt=1.064 min.

### Step 7: The Synthesis of N-(4-Amino-1H-pyrazolo[4,3-c]pyridin-7-yl)-N′-[1-[2-chloro-4-(trifluoromethyl)phenyl]ethyl]-N′-methyl-oxamide

Hydrogen chloride solution 4.0M in dioxane (10.10 g, 27.70 mmol, 10 mL, 10% purity) was added to the solution of N-(4-amino-2-tetrahydropyran-2-yl-pyrazolo[4,3-c]pyridin-7-yl)-N′-[1-[2-chloro-4-(trifluoromethyl)phenyl]ethyl]-N′-methyl-oxamide (2.5 g, 4.76 mmol) in Methanol (25 mL). Resulting reaction mixture was stirred at 25° C. for 18 hr. Then, volatiles were removed under reduced pressure and residue was subjected to HPLC (5-30% 0-5 min H₂O/MeCN/0.1% FA, flow: 30 mL/min, column: Chromatorex 18 SMB100-5T 100×19 mm 5 μm), affording N-(4-amino-1H-pyrazolo[4,3-c]pyridin-7-yl)-N′-[1-[2-chloro-4-(trifluoromethyl)phenyl]ethyl]-N′-methyl-oxamide (0.86 g, 1.95 mmol, 40.96% yield).

LCMS (ESI): [M+H]⁺m/z: calcd 441.2; found 441.0; Rt=1.083 min.

### Step 8: The Synthesis of(S)-N1-(4-amino-1H-pyrazolo[4,3-c]pyridin-7-yl)-N2-(1-(2-chloro-4-(trifluoromethyl)phenyl)ethyl)-N2-methyloxalamide (Compound 167) and (R)—N¹-(4-amino-1H-pyrazolo[4,3-c]pyridin-7-yl)-N2-(1-(2-chloro-4-(trifluoromethyl)phenyl)ethyl)-N2-methyloxalamide (Compound 187)

N-(4-Amino-1H-pyrazolo[4,3-c]pyridin-7-yl)-N′-[1-[2-chloro-4-(trifluoromethyl)phenyl]ethyl]-N′-methyl-oxamide (0.86 g, 1.95 mmol) was divided into enantiomers by Chiral HPLC (Column: CHIRALPAK IC (250×30 mm, 10 mkm); Mobile phase: Hexane (0.1% DEA): IPA: MeOH, 60:20:20, Flow Rate: 30 mL/min; Column Temperature: 24° C., RetTime (isomer A)=12.7 min; RetTime (isomer B)=21.98 min), affording: Compound 167-(S)-N1-(4-amino-1H-pyrazolo[4,3-c]pyridin-7-yl)-N2-(1-(2-chloro-4-(trifluoromethyl)phenyl)ethyl)-N2-methyloxalamide (255 mg, 578.49 μmol, 59.30% yield) (ret.time=11.0 min (analytical), 12.70 min (preparative)) and Compound 187-(R)—N¹-(4-amino-1H-pyrazolo[4,3-c]pyridin-7-yl)-N2-(1-(2-chloro-4-(trifluoromethyl)phenyl)ethyl)-N2-methyloxalamide (303 mg, 687.38 μmol, 70.47% yield)(ret.time=15.7 min (analytical), 21.98 min (preparative)).

The absolute stereochemistry of the two compounds was independently confirmed.

### Compound 167:

Yield: 255.0 mg (59.30%)

RT (Chiralpak AD-H (250*4.6 mm, 5 mkm); Hexane (0.1% EDTA): IPA: MeOH, 50:25:25, Flow Rate: 0.6 mL/min)=10.474 min.

¹H NMR (600 MHz, dmso) 8 1.13-1.75 (m, 3H), 2.56-2.94 (m, 3H), 5.31-5.87 (m, 1H), 6.54-6.86 (m, 2H), 7.18-7.93 (m, 4H), 8.09-8.22 (m, 1H), 9.60-10.56 (m, 1H), 12.57-13.28 (m, 1H).

LCMS (ESI): [M+H]⁺m/z: calcd 441.2; found 441.2; Rt=2.328 min.

### Compound 187:

Yield: 303.0 mg (70.47%)

RT (Chiralpak AD-H (250*4.6 mm, 5 mkm); Hexane (0.1% EDTA): IPA: MeOH, 50:25:25, Flow Rate: 0.6 mL/min)=14.493 min.

[α]²¹D=+91.10 deg (c=0.2 g/100 mL, MeOH).

¹H NMR (600 MHz, dmso) δ 1.16-1.72 (m, 3H), 2.58-2.92 (m, 3H), 5.29-5.89 (m, 1H), 6.50-6.97 (m, 2H), 7.18-7.93 (m, 4H), 8.10-8.29 (m, 1H), 9.60-10.54 (m, 1H), 12.46-13.36 (m, 1H).

LCMS (ESI): [M+H]⁺m/z: calcd 441.2; found 441.2; Rt=2.313 min.

### Example 163. Synthesis of N-(4-Amino-1H-pyrazolo[4,3-c]pyridin-7-yl)-N′-cyclobutyl-N′-[1-[4-(trifluoromethyl)phenyl]ethyl]oxamide (Compound 192)

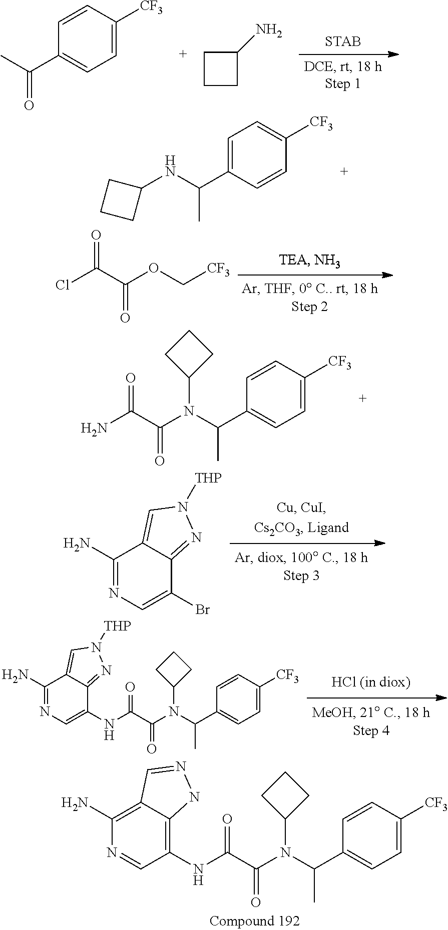

### Step 1: The Synthesis of N-[1-[4-(trifluoromethyl)phenyl]ethyl]cyclobutanamine

A solution of 1-[4-(trifluoromethyl)phenyl]ethanone (0.5 g, 2.66 mmol), cyclobutanamine (378.01 mg, 5.32 mmol, 453.79 μL) in DCE (30 mL) was stirred at room temperature for 30 min, then Sodium triacetoxyborohydride (1.69 g, 7.97 mmol, 1.57 mL) was added in one portion and the resulting mixture was stirred for 18 hr at room temperature. The reaction mixture was taken up with NaOH (15 mL, 5% aq.) and extracted with DCM (2*15 mL). The combined organic layers were dried over Na₂SO₄ and evaporated in vacuo to obtain N-[1-[4-(trifluoromethyl)phenyl]ethyl]cyclobutanamine (0.5 g, 2.06 mmol, 77.34% yield).

LCMS (ESI): [M+H]⁺m/z: calcd 244.0; found 244.0; Rt=0.962 min.

### Step 2: The Synthesis of N′-Cyclobutyl-N′-[1-[4-(trifluoromethyl)phenyl]ethyl]oxamide

To a solution of N-[1-[4-(trifluoromethyl)phenyl]ethyl]cyclobutanamine (0.5 g, 2.06 mmol) and TEA (415.96 mg, 4.11 mmol, 572.95 μL) in THF (30 mL) was added 2,2,2-trifluoroethyl 2-chloro-2-oxo-acetate (587.33 mg, 3.08 mmol) dropwise at 0° C. under argon. The reaction mixture was then stirred for 12 hr at r.t. Then ammonia (292.20 mg, 17.16 mmol, 339.77 μL) was bubbled through for 10 min at 0° C. The reaction mixture was then stirred for 12 hr at r.t. The reaction mixture was filtered off and the filtrate was evaporated in vacuo to give N′-cyclobutyl-N′-[1-[4-(trifluoromethyl)phenyl]ethyl]oxamide (0.4 g, 1.27 mmol, 61.92% yield).

LCMS (ESI): [M−H]⁺m/z: calcd 313.0; found 313.0; Rt=1.095 min.

### Step 3: The Synthesis of N-(4-Amino-2-tetrahydropyran-2-yl-pyrazolo[4,3-c]pyridin-7-yl)-N′-cyclobutyl-N′-[1-[4-(trifluoromethyl)phenyl]ethyl]oxamide

N′-Cyclobutyl-N′-[1-[4-(trifluoromethyl)phenyl]ethyl]oxamide (246.80 mg, 785.24 μmol), 7-bromo-2-tetrahydropyran-2-yl-pyrazolo[4,3-c]pyridin-4-amine (350 mg, 1.18 mmol), Cu(5.29 mg, 83.24 μmol), CuI (44.86 mg, 235.57 μmol, 7.98 μL), Cesium carbonate (383.77 mg, 1.18 mmol, 167.58 μL) and (1R,2R)—N1,N2-dimethylcyclohexane-1,2-diamine (33.51 mg, 235.57 μmol) were mixed in dioxane (8.28 mL), purged with Ar for 2 minutes and then heated in the sealed tube at 100° C. for 18 hr. Final mixture was filtered and the filtrate was evaporated in vacuo to afford N-(4-amino-2-tetrahydropyran-2-yl-pyrazolo[4,3-c]pyridin-7-yl)-N′-cyclobutyl-N′-[1-[4-(trifluoromethyl)phenyl]ethyl]oxamide (0.5 g, crude) as brown solid, which was used directly in the next step.

LCMS (ESI): [M+H]⁺m/z: calcd 531.2; found 531.2; Rt=1.184 min.

### Step 4: The Synthesis of N-(4-Amino-1H-pyrazolo[4,3-c]pyridin-7-yl)-N′-cyclobutyl-N′-[1-[4-(trifluoromethyl)phenyl]ethyl]oxamide (Compound 192)

To a solution of N-(4-amino-2-tetrahydropyran-2-yl-pyrazolo[4,3-c]pyridin-7-yl)-N′-cyclobutyl-N′-[1-[4-(trifluoromethyl)phenyl]ethyl]oxamide (0.5 g, 376.97 μmol) in methanol (10 mL) was added Hydrogen chloride solution 3.0M in dioxane (2.75 g, 7.54 mmol, 2.62 mL, 10% purity) at 21° C. The resulting mixture was left to stir for 18 hr. The resulting mixture was evaporated to dryness. The residue was purified by RP-HPLC (column: Chromatorex 18 SNB100-5T 100×19 5 μm; May 10, 2040% 0-1.3-6.3 min H₂O/MeCN/0.1% FA, flow: 30 mL/min) to give Compound 192 N-(4-amino-1H-pyrazolo[4,3-c]pyridin-7-yl)-N′-cyclobutyl-N′-[1-[4-(trifluoromethyl)phenyl]ethyl]oxamide (108 mg, 241.92 μmol, 64.17% yield).

¹H NMR (600 MHz, dmso) δ 1.44-1.60 (m, 2H), 1.62-2.13 (m, 4H), 2.15-2.34 (m, 1H), 2.76-2.86 (m, 1H), 3.61-3.65 (m, 1H), 4.38-4.82 (m, 1H), 4.86-5.40 (m, 1H), 6.60-7.00 (m, 2H), 7.17-7.77 (m, 4H), 8.09-8.23 (m, 2H), 9.46-10.63 (m, 1H), 12.35-13.55 (m, 1H).

LCMS (ESI): [M+H]⁺m/z: calcd 447.2; found 447.2; Rt=2.922 min.

### Example 164. Synthesis of N-(4-Amino-2H-pyrazolo[4,3-c]pyridin-7-yl)-N′-methyl-N′—[(1S)-1-[2,4-bis(trifluoromethyl)phenyl]ethyl]oxamide (Compound 169) and N-(4-Amino-2H-pyrazolo[4,3-c]pyridin-7-yl)-N′-methyl-N′—[(1R)-1-[2,4-bis(trifluoromethyl)phenyl]ethyl]oxamide (Compound 193)

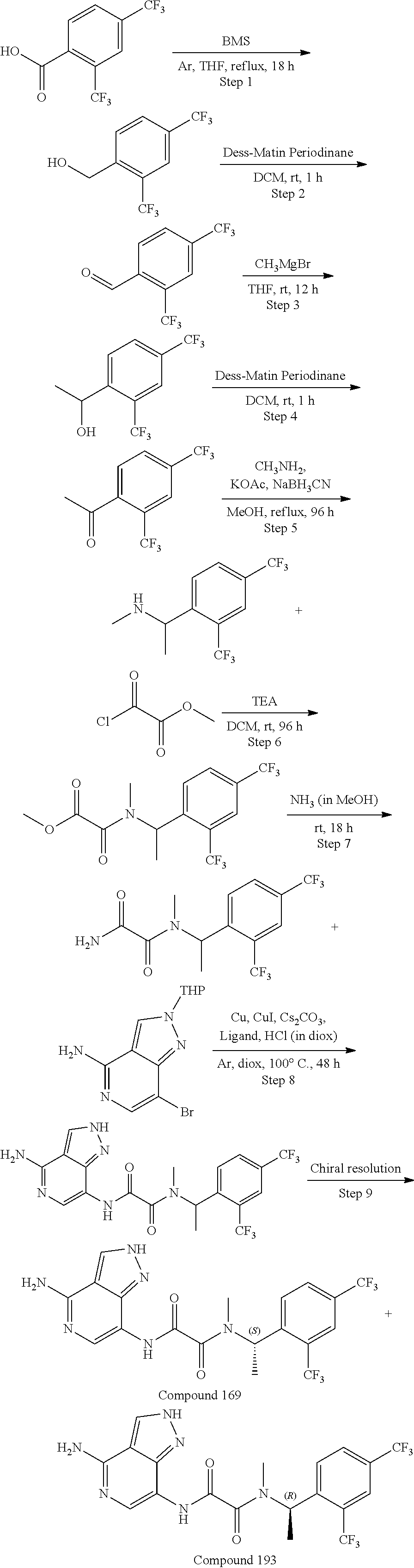

### Step 1: The Synthesis of [2,4-Bis(trifluoromethyl)phenyl]methanol

To a solution of 2,4-bis(trifluoromethyl)benzoic acid (10 g, 38.74 mmol) in THF (250 mL) was added Borane dimethyl sulfide complex (12.42 g, 163.43 mmol, 15.5 mL) under Ar atmosphere. After stirring at reflux for 18 hr the resulting mixture was quenched with MeOH, refluxed for 1 hr, evaporated to dryness, and reevaporated with MeOH to afford [2,4-bis(trifluoromethyl)phenyl]methanol (9.95 g, crude) as a light-yellow gum.

¹H NMR (500 MHz, CDCl₃) δ (ppm) 2.03 (s, 1H), 4.96 (s, 2H), 7.83-7.94 (m, 3H).

### Step 2: The Synthesis of 2,4-Bis(trifluoromethyl)benzaldehyde

To a solution of [2,4-bis(trifluoromethyl)phenyl]methanol (9.95 g, 36.68 mmol) in DCM (250 mL) was added Dess-Martin Periodinane (18.67 g, 44.02 mmol) in one portion. The resulting mixture was stirred for 1 h at rt. The reaction mixture was poured into a solution containing Na₂S₂O₃ and Na₂CO₃ (2:1 by weight), stirred for 30 min, and DCM was dried and evaporated. 2,4-Bis (trifluoromethyl)benzaldehyde (8.5 g, 35.11 mmol, 95.71% yield) was obtained as a yellow oil.

¹H NMR (500 MHz, CDCl₃) δ (ppm) 7.99 (d, 1H), 8.04 (s, 1H), 8.26 (d, 1H), 10.44 (s, 1H).

### Step 3: The Synthesis of 1-[2,4-Bis(trifluoromethyl)phenyl]ethanol

Bromo (methyl) magnesium (6.28 g, 52.66 mmol, 2.1 mL) was added to the solution of 2,4-bis(trifluoromethyl)benzaldehyde (8.5 g, 35.11 mmol, 5.74 mL) in THF (250 mL) maintaining the internal temperature below 25° C. The resulting reaction mixture was allowed to stir at room temperature for 12 hr, then quenched with saturated NH₄Cl solution and extracted with EtOAc (2×125 mL). The combined organic layer was dried over Na₂SO₄, filtered, and concentrated under reduced pressure to obtain product 1-[2,4-bis(trifluoromethyl)phenyl]ethanol (8.5 g, 32.93 mmol, 93.79% yield) as a light-yellow oil, which was used in the next step reaction without any further purification.

¹H NMR (500 MHz, CDCl₃) δ (ppm) 1.48 (d, 1H), 2.14 (br s, 1H), 5.35 (q, 1H), 7.81 (d, 1H), 7.84 (s, 1H), 7.99 (d, 1H).

### Step 4: The Synthesis of 1-[2,4-Bis(trifluoromethyl)phenyl]ethenone

To a solution of 1-[2,4-bis(trifluoromethyl)phenyl]ethanol (8.5 g, 32.93 mmol) in DCM (125 mL) was added Dess-Martin Periodinane (16.76 g, 39.51 mmol) in one portion. The resulting mixture was stirred for 1 h at rt. The reaction mixture was poured into a solution containing Na₂S₂O₃ and Na₂CO₃ (2:1 by weight), stirred for 30 min, and DCM was dried and evaporated. 1-[2,4-Bis(trifluoromethyl)phenyl]ethanone (8 g, 31.23 mmol, 94.86% yield) was obtained as a light-yellow oil.

¹H NMR (400 MHz, CDCl₃) δ (ppm) 2.57 (s, 3H), 7.55 (d, 1H), 7.86 (d, 1H), 7.93 (s, 1H).

### Step 5: The Synthesis of 1-[2,4-Bis(trifluoromethyl)phenyl]-N-methyl-ethanamine

To a solution of 1-[2,4-bis(trifluoromethyl)phenyl]ethanone (1 g, 3.90 mmol) in MeOH (49.83 mL) was added Sodium cyanoborohydride (368.00 mg, 5.86 mmol), Potassium Acetate (3.83 g, 39.04 mmol, 2.44 mL) and N-methylamine (2.64 g, 39.04 mmol, 2.93 mL, HCl). The mixture was refluxed for 96 h. The reaction was quenched by adding water (100 mL) and was extracted with EtOAc (100 mL×2). The combined organic layers were washed with brine (2×50 mL), dried over anhydrous Na₂SO₄ and concentrated under reduced pressure to give 1-[2,4-bis(trifluoromethyl)phenyl]-N-methyl-ethanamine (0,2 g, 737.46 μmol, 18.89% yield) as a yellow oil.

LCMS (ESI): [M+H]⁺m/z: calcd 272.2; found 272.2; Rt=1.996 min.

### Step 6: The Synthesis of Methyl 2-[1-[2,4-bis(trifluoromethyl)phenyl]ethyl-methyl-amino]-2-oxo-acetate

To a solution of 1-[2,4-bis(trifluoromethyl)phenyl]-N-methyl-ethanamine (0,2 g, 737.46 μmol) and TEA (111.94 mg, 1.11 mmol, 154.18 μL) in DCM (14.95 mL) was added methyl 2-chloro-2-oxo-acetate (90.34 mg, 737.46 μmol) at rt. After stirring at rt for 1 hr the resulting mixture was washed with water, dried, and evaporated to dryness to give methyl 2-[1-[2,4-bis(trifluoromethyl)phenyl]ethyl-methyl-amino]-2-oxo-acetate (0.2 g, 559.84 μmol, 75.91% yield) as an off-white solid and was used in the next step without further purification.

LCMS (ESI): [M+H]⁺m/z: calcd 358.2; found 358.2; Rt=3.422 min.

### Step 7: The Synthesis of N′-[1-[2,4-Bis(trifluoromethyl)phenyl]ethyl]-N′-methyl-oxamide

Ammonia (9.53 mg, 559.84 μmol, 11.09 μL) was bubbled through a solution of methyl 2-[1-[2,4-bis(trifluoromethyl)phenyl]ethyl-methyl-amino]-2-oxo-acetate (0.2 g, 559.84 μmol) in MeOH (15 mL) at rt. After stirring for 18 hr, the reaction mixture was evaporated to dryness, the residue was purified by HPLC (13-20-45-100% 0-2-7-7.1 min; 30 mL/min water-acetonitrile (loading pump 4 mL/min acetonitrile); column SunFireC18 19*100 mm (L)) to give N′-[1-[2,4-bis(trifluoromethyl)phenyl]ethyl]-N′-methyl-oxamide (47 mg, 137.33 μmol, 24.53% yield) as a yellow solid.

LCMS (ESI): [M−H]⁺m/z: calcd 341.2; found 341.2; Rt=2.936 min.

### Step 8: The Synthesis of N-(4-Amino-2H-pyrazolo[4,3-c]pyridin-7-yl)-N′-[1-[2,4-bis(trifluoromethyl)phenyl]ethyl]-N′-methyl-oxamide

N′-[1-[2,4-Bis(trifluoromethyl)phenyl]ethyl]-N′-methyl-oxamide (0.047 g, 109.87 μmol), 7-bromo-2-tetrahydropyran-2-yl-pyrazolo[4,3-c]pyridin-4-amine (32.65 mg, 109.87 μmol), Copper (349.10 μg, 5.49 μmol), Copper (I) iodide (10.46 mg, 54.93 μmol, 1.86 μL), rac-(1R,2R)—N1,N2-dimethylcyclohexane-1,2-diamine (11.72 mg, 82.40 μmol) and Cesium carbonate (71.59 mg, 219.73 μmol) were mixed in Dioxane (4 mL). The resulting mixture was purged with argon for 30 sec. The vials were sealed and heated at 100° C. for 48 hr. The reaction mixture was cooled and filtered. The filter cake was washed with MeOH (10 mL) and the filtrate was concentrated in vacuo and redissolved in MeOH (5 mL)/Hydrogen chloride solution 4.0M in dioxane (4.01 mg, 109.87 μmol, 4 mL) mixture. After 1 hr the reaction mixture was filtered, rinsed with MeOH, and evaporated. The residue was purified by HPLC (1st run: 33-40-80% 0-2-7-7.1 min; 30 mL/min water-MeOH+NH3 (loading pump 4 mL/min MeOH+NH3); column Xbridge C18 5 μM 19*100 mm; 2nd run: 53-60-90-100% 0-2-7-7.1 min; 30 mL/min water-MeOH (loading pump 4 mL/min MeOH); column Kinetex PFP 5 μM 21.2*100 mm (R)) to give N-(4-amino-2H-pyrazolo[4,3-c]pyridin-7-yl)-N′-[1-[2,4-bis(trifluoromethyl)phenyl]ethyl]-N′-methyl-oxamide (6.4 mg, 13.49 μmol, 12.28% yield) as a beige solid.

LCMS (ESI): [M+H]⁺m/z: calcd 475.2; found 475.2; Rt=1.599 min.

### Step 9: The Synthesis of N-(4-Amino-2H-pyrazolo[4,3-c]pyridin-7-yl)-N′-methyl-N′—[(1S)-1-[2,4-bis(trifluoromethyl)phenyl]ethyl]oxamide (Compound 169) and N-(4-Amino-2H-pyrazolo[4,3-c]pyridin-7-yl)-N′-methyl-N′—[(1R)-1-[2,4-bis(trifluoromethyl)phenyl]ethyl]oxamide (Compound 193)

The mixture of diastereomers was separated by chiral chromatography (Column: CHIRALPAK AD (250×30 mm, 10 mkm)-II; Mobile Phase: Hexane (0.1% DEA): IPA: MeOH, 50:25:25 Flow Rate: 30 ml/min) to obtain N-(4-amino-2H-pyrazolo[4,3-c]pyridin-7-yl)-N′-methyl-N′—[(1S)-1-[2,4-bis(trifluoromethyl)phenyl]ethyl]oxamide (2.31 mg, 4.87 μmol, 36.09% yield) and N-(4-amino-2H-pyrazolo[4,3-c]pyridin-7-yl)-N′-methyl-N′—[(1R)-1-[2,4-bis(trifluoromethyl)phenyl]ethyl]oxamide (3.29 mg, 6.94 μmol, 51.41% yield)

### Compound 169:

Yield: 2.31 mg (36.09%)

Analytical: RT (Chiralpak AD-H (250×4.6 mm, 5 mkm); Hexane (0.1% EDA): IPA: MeOH, 50:25:25 Flow Rate: 0.6 mL/min)=7.1372 min;

Preparative: RT (CHIRALPAK AD (250×30 mm, 10 mkm); Hexane (0.1% DEA): IPA: MeOH, 50:25:25 Flow Rate: 30 mL/min)=7.1562 min;

LCMS (ESI): [M+H]⁺m/z: calcd 475.0; found 475.0; Rt=3.977 min.

### Compound 167:

Yield: 3.29 mg (51.41%)

Analytical: RT (Chiralpak AD-H (250×4.6 mm, 5 mkm); Hexane (0.1% EDA): IPA: MeOH, 50:25:25 Flow Rate: 0.6 mL/min)=24.532 min.

Preparative: RT (CHIRALPAK AD (250×30 mm, 10 mkm); Hexane (0.1% DEA): IPA: MeOH, 50:25:25 Flow Rate: 30 mL/min)=14.237 min.

LCMS (ESI): [M+H]⁺m/z: calcd 475.0; found 475.0; Rt=4.068 min.

The absolute stereochemistry of the two compounds was independently confirmed.

### Example 165. Synthesis of N-(4-Amino-1H-pyrazolo[4,3-c]pyridin-7-yl)-N′-ethyl-N′—[(1R)-1-[2-fluoro-4-(1,1,2,2,2-pentafluoroethyl)phenyl]ethyl]oxamide (Compound 103) and N-(4-Amino-1H-pyrazolo[4,3-c]pyridin-7-yl)-N′-ethyl-N′—[(1S)-1-[2-fluoro-4-(1,1,2,2,2-pentafluoroethyl)phenyl]ethyl]oxamide (Compound 134)

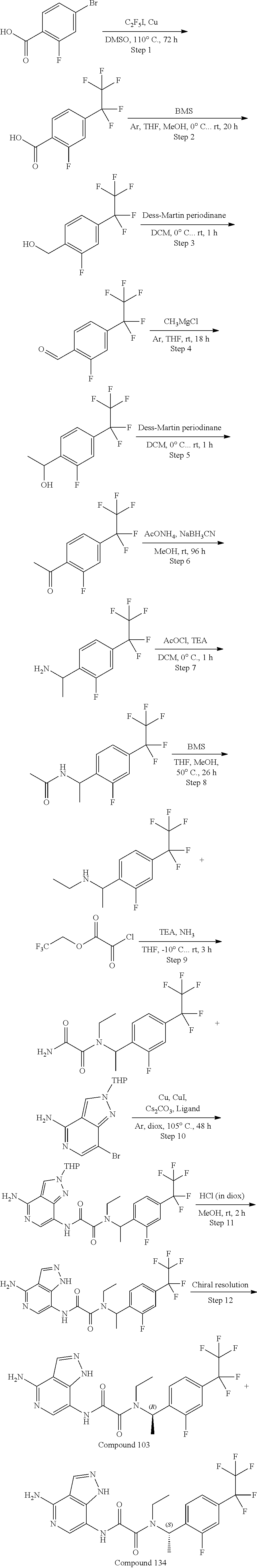

Steps 1-3 were Described for Compound 189

### Step 4: The Synthesis of 1-[2-Fluoro-4-(1,1,2,2,2-pentafluoroethyl)phenyl]ethanol

Methylmagnesium chloride (12.64 g, 37.17 mmol, 12.27 mL, 22% purity) was added slowly under argon at 0° C. to a stirred solution of 2-fluoro-4-(1,1,2,2,2-pentafluoroethyl)benzaldehyde (6 g, 24.78 mmol) in THF (100 mL). The resulting mixture was allowed to warm to 25° C. and stirred for 18 hr. The reaction mixture was quenched with saturated aqueous ammonium chloride solution. THF was removed in vacuo, the residual aqueous phase was extracted with MTBE (2*70 mL). The combined organic extracts were washed successively with water (20 mL) and brine (20 mL), dried over sodium sulfate and concentrated in vacuo to afford 1-[2-fluoro-4-(1,1,2,2,2-pentafluoroethyl)phenyl]ethanol (6.4 g, 24.79 mmol, 100.04% yield) as light-yellow oil.

¹H NMR (500 MHz, CDCl₃) δ (ppm) 1.52 (s, 3H), 5.24 (q, 1H), 7.26 (s, 1H), 7.41 (m, 1H), 7.67 (m, 1H).

### Step 5: The Synthesis of 1-[2-Fluoro-4-(1,1,2,2,2-pentafluoroethyl)phenyl]ethenone

1, 1-Bis(acetyloxy)-3-oxo-3H-1 lambda 5,2-benziodaoxol-1-yl acetate (5.91 g, 13.94 mmol) was added in one portion to a cooled to 0° C. solution of 1-[2-fluoro-4-(1,1,2,2,2-pentafluoroethyl)phenyl]ethanol (3 g, 11.62 mmol) in dichloromethane (100 mL). The resulting mixture was allowed to warm to 25° C. over 1 hr period. The reaction mixture was carefully neutralized with 5% aqueous sodium hydrogen carbonate solution. The resulting slurry was filtered, the filtercake was washed with dichloromethane (2*20 ml) and discarded. The combined filtrate was transferred to a separatory funnel, the dichloromethane layer was separated, dried over sodium sulfate and concentrated in vacuo. The residue was triturated with hexane (100 mL), stirred for 10 min, and then filtered. The filtercake was additionally washed with hexane (2*20 mL) and then discarded. The combined hexane filtrate was concentrated in vacuo to afford 1-[2-fluoro-4-(1,1,2,2,2-pentafluoroethyl)phenyl]ethanone (2.7 g, 10.54 mmol, 90.71% yield) as yellow oil.

¹H NMR (400 MHz, CDCl₃) δ (ppm) 2.67 (s, 3H), 7.38-7.47 (m, 2H), 7.98 (t, 1H).

### Step 6: The Synthesis of 1-[2-Fluoro-4-(1,1,2,2,2-pentafluoroethyl)phenyl]ethanamine

Sodium cyanoborohydride (932.28 mg, 14.84 mmol) was added in one portion to a stirred mixture of 1-[2-fluoro-4-(1,1,2,2,2-pentafluoroethyl)phenyl]ethanone (1.9 g, 7.42 mmol) and Ammonium acetate (5.72 g, 74.18 mmol, 4.89 mL) in methanol (40 mL) at 25° C. The reaction mixture was stirred at 25° C. for 96 hr, and then concentrated in vacuo. The residue was diluted with 2N aqueous hydrochloric acid (20 mL). The resulting mixture was filtered through a cotton pad to remove oily impurities. The filtrate was basified with 10% aqueous sodium hydroxide solution to pH 10-11 and then extracted with dichloromethane (2*30 mL). The combined organic extracts were dried over sodium sulfate and concentrated in vacuo to afford 1-[2-fluoro-4-(1,1,2,2,2-pentafluoroethyl)phenyl]ethanamine (700 mg, 2.72 mmol, 36.69% yield) as light-brown oil.

LCMS (ESI): [M+H]⁺m/z: calcd 258.2; found 258.2; Rt=0.925 min.

### Step 7: The Synthesis of N-[1-[2-Fluoro-4-(1,1,2,2,2-pentafluoroethyl)phenyl]ethyl]acetamide

Acetyl chloride (299.13 mg, 3.81 mmol, 231.16 μL) was added in one portion to a stirred solution of 1-[2-fluoro-4-(1, 1,2,2,2-pentafluoroethyl)phenyl]ethanamine (700 mg, 2.72 mmol) and triethyl amine (826.27 mg, 8.17 mmol, 1.14 mL) in dichloromethane (25 mL) at 0° C. The reaction mixture was stirred at 0° C. for 1 hr, then transferred to a separatory funnel and washed successively with water (20 mL) and brine (15 mL). The organic layer was separated, dried over sodium sulfate and concentrated in vacuo to afford crude N-[1-[2-fluoro-4-(1,1,2,2,2-pentafluoroethyl)phenyl]ethyl]acetamide (750 mg, 2.51 mmol, 92.09% yield) as light-brown gum, which was used directly in the next step.

LCMS (ESI): [M+H]⁺m/z: calcd 300.2; found 300.0; Rt=1.150 min.

### Step 8: The Synthesis of N-Ethyl-1-[2-fluoro-4-(1,1,2,2,2-pentafluoroethyl)phenyl]ethanamine

Borane dimethyl sulfide complex (952.13 mg, 12.53 mmol, 1.19 mL) was added dropwise under argon to a cooled to 0° C. solution of N-[1-[2-fluoro-4-(1,1,2,2,2-pentafluoroethyl)phenyl]ethyl]acetamide (750 mg, 2.51 mmol) in THF (25 mL). The resulting mixture was allowed to warm to 50° C. and stirred for 24 hr. The reaction mixture was again cooled to 0° C. and methanol (20 mL) was added dropwise over 0.5 hr. Then 2N aqueous hydrochloric acid (10 mL) was added slowly, and the resulting mixture was gradually warmed to 50° C. and stirred for 2 hr. The reaction mixture was cooled down and concentrated in vacuo, the residue was basified with 10% aqueous sodium hydroxide solution to pH 10-11 and extracted with MTBE (2*80 mL). The combined organic extracts were dried over sodium sulfate and concentrated in vacuo to afford crude N-ethyl-1-[2-fluoro-4-(1,1,2,2,2-pentafluoroethyl)phenyl]ethanamine (700 mg, 2.45 mmol, 97.91% yield) as yellow oil, which was used directly in the next step.

LCMS (ESI): [M+H]⁺m/z: calcd 286.2; found 286.2; Rt=0.743 min.

### Step 9: The Synthesis of N′-Ethyl-N′-[1-[2-fluoro-4-(1,1,2,2,2-pentafluoroethyl)phenyl]ethyl]oxamide

2,2,2-Trifluoroethyl 2-chloro-2-oxo-acetate (729.86 mg, 3.83 mmol) was added slowly to a cooled to −10° C. mixture of N-ethyl-1-[2-fluoro-4-(1,1,2,2,2-pentafluoroethyl)phenyl]ethanamine (700 mg, 2.45 mmol) and triethyl amine (1.22 g, 12.02 mmol, 1.68 mL) in THF (51.06 mL). The resulting mixture was allowed to warm to 25° C. and stirred for 2 hr. Then gaseous ammonia (41.79 mg, 2.45 mmol, 48.60 μL) was vigorously bubbled through it at 25° C. for 1 hr. The resulting mixture was filtered to remove ammonium chloride, the filtercake was washed with THF (2*20 mL) and discarded. The combined filtrate was concentrated in vacuo to afford crude N′-ethyl-N′-[1-[2-fluoro-4-(1,1,2,2,2-pentafluoroethyl)phenyl]ethyl]oxamide (800 mg, 2.25 mmol, 91.50% yield) as red gum, which was used directly in the next step.

### Step 10: The Synthesis of N-(4-Amino-2-tetrahydropyran-2-yl-pyrazolo[4,3-c]pyridin-7-yl)-N′-ethyl-N′-[1-[2-fluoro-4-(1,1,2,2,2-pentafluoroethyl)phenyl]ethyl]oxamide

A mixture of N′-ethyl-N′-[1-[2-fluoro-4-(1,1,2,2,2-pentafluoroethyl)phenyl]ethyl]oxamide (400 mg, 1.12 mmol), 7-bromo-2-tetrahydropyran-2-yl-pyrazolo[4,3-c]pyridin-4-amine (417.04 mg, 1.40 mmol), copper (5 mg, 78.68 μmol), Copper (I) iodide (155.65 mg, 817.27 μmol, 27.70 μL), cesium carbonate (548.73 mg, 1.68 mmol) and rac-(1R,2R)—N1,N2-dimethylcyclohexane-1,2-diamine (155.65 mg, 1.09 mmol) in 1,4-dioxane (7 mL) was stirred in a sealed vial under argon at 105° C. for 48 hr. The resulting mixture was cooled down and filtered. The filtercake was washed successively with THF (2*5 mL) and dichloromethane (3*5 mL). The combined filtrate was concentrated in vacuo to afford crude N-(4-amino-2-tetrahydropyran-2-yl-pyrazolo[4,3-c]pyridin-7-yl)-N′-ethyl-N′-[1-[2-fluoro-4-(1,1,2,2,2-pentafluoroethyl)phenyl]ethyl]oxamide (1.3 g, crude) as brown gum, which was used directly in the next step.

LCMS (ESI): [M+H]⁺m/z: calcd 573.2; found 573.2; Rt=3.172 min.

### Step 11: The Synthesis of N-(4-Amino-1H-pyrazolo[4,3-c]pyridin-7-yl)-N′-ethyl-N′-[1-[2-fluoro-4-(1,1,2,2,2-pentafluoroethyl)phenyl]ethyl]oxamide

Hydrogen chloride solution 4.0M in dioxane (7.40 g, 28.20 mmol, 7.05 mL, 13.9% purity) was added to a stirred solution of crude from previous step N-(4-amino-2-tetrahydropyran-2-yl-pyrazolo[4,3-c]pyridin-7-yl)-N′-ethyl-N′-[1-[2-fluoro-4-(1, 1,2,2,2-pentafluoroethyl)phenyl]ethyl]oxamide (1.3 g, 2.27 mmol) in methanol (2.95 mL) at 25° C. The resulting solution was stirred at 25° C. for 2 hr, then concentrated to dryness in vacuo to afford crude product, which was purified by reverse phase HPLC (column: Agilent 5 PrepC18 100x30 mm 5 μm; mobile phase: 20-20-65% 0-1-5 min H₂O/MeCN/0.2% FA; flow: 30 mL/min; (loading pump 4 mL/min acetonitrile)) to afford N-(4-amino-1H-pyrazolo[4,3-c]pyridin-7-yl)-N′-ethyl-N′-[1-[2-fluoro-4-(1, 1,2,2,2-pentafluoroethyl)phenyl]ethyl]oxamide (150 mg, 307.13 μmol, 13.53% yield) as grey solid.

LCMS (ESI): [M+H]⁺m/z: calcd 489.2; found 489.0; Rt=0.974 min.

### Step 12: The Synthesis of N-(4-Amino-1H-pyrazolo[4,3-c]pyridin-7-yl)-N′-ethyl-N′—[(1R)-1-[2-fluoro-4-(1,1,2,2,2-pentafluoroethyl)phenyl]ethyl]oxamide (Compound 103) and N-(4-Amino-1H-pyrazolo[4,3-c]pyridin-7-yl)-N′-ethyl-N′—[(1S)-1-[2-fluoro-4-(1,1,2,2,2-pentafluoroethyl)phenyl]ethyl]oxamide (Compound 134)

Racemic N-(4-amino-1H-pyrazolo[4,3-c]pyridin-7-yl)-N′-ethyl-N′-[1-[2-fluoro-4-(1,1,2,2,2-pentafluoroethyl)phenyl]ethyl]oxamide (150 mg, 307.13 μmol) was submitted to preparative chiral HPLC (column: CHIRALPAK IC (250×20 mm, 5 mkm)-II; Mobile phase: Hexane-IPA-MeOH, 70-15-15;

Flow rate: 14 ml/min) to afford Compound 103-N-(4-amino-1H-pyrazolo[4,3-c]pyridin-7-yl)-N′-ethyl-N′—[-(1R)-1-[2-fluoro-4-(1,1,2,2,2-pentafluoroethyl)phenyl]ethyl]oxamide (83 mg, 169.95 μmol, 55.33% yield)(RetTime=11.514 min.) as light-yellow solid; and Compound 134 —N-(4-amino-1H-pyrazolo[4,3-c]pyridin-7-yl)-N′-ethyl-N′—[(1S)-1-[2-fluoro-4-(1,1,2,2,2-pentafluoroethyl)phenyl]ethyl]oxamide (91 mg, 186.33 μmol, 60.67% yield)(RetTime=16.982 min.) as light-yellow solid.

The absolute stereochemistry of the two compounds was independently confirmed.

### Compound 103:

Yield: 83.0 mg (55.33%)

RT (Chiralpak IC (250*4.6 mm, 5 mkm); Hexane (0.1% EDTA): IPA: MeOH, 70:15:15, Flow Rate: 0.6 mL/min)=11.656 min.

¹H NMR (600 MHz, dmso) 8 0.85-1.12 (m, 3H), 1.60-1.68 (m, 3H), 2.80-2.85 (m, 1H), 3.47-3.51 (m, 1H), 5.60-5.96 (m, 1H), 6.60-6.72 (m, 2H), 7.54-7.60 (m, 1H), 7.60-7.68 (m, 1H), 7.68-7.75 (m, 1H), 7.77-7.88 (m, 1H), 8.18-8.29 (m, 1H), 10.21-10.60 (m, 1H), 12.55-13.18 (m, 1H).

LCMS (ESI): [M+H]⁺m/z: calcd 489.2; found 489.0; Rt=3.213 min.

### Compound 134:

Yield: 91.0 mg (60.67%)

RT (Chiralpak IC (250*4.6 mm, 5 mkm); Hexane (0.1% EDTA): IPA: MeOH, 70:15:15, Flow Rate: 0.6 mL/min)=17.266 min

¹H NMR (600 MHz, dmso) 8 0.84-1.14 (m, 3H), 1.62-1.73 (m, 3H), 2.86-2.98 (m, 1H), 3.46-3.53 (m, 1H), 5.62-5.95 (m, 1H), 6.74 (s, 2H), 7.56-7.61 (m, 1H), 7.62-7.66 (m, 1H), 7.66-7.76 (m, 1H), 7.79-7.89 (m, 1H), 8.16-8.24 (m, 1H), 10.21-10.58 (m, 1H), 12.53-13.33 (m, 1H).

LCMS (ESI): [M+H]⁺m/z: calcd 489.2; found 489.0; Rt=3.182 min.

### Example 166. Synthesis of N-(7-Amino-1-methyl-pyrazolo[3,4-c]pyridin-4-yl)-N′-methyl-N′—[(1S)-1-[4-(trifluoromethyl)phenyl]ethyl]oxamide (Compound 79)

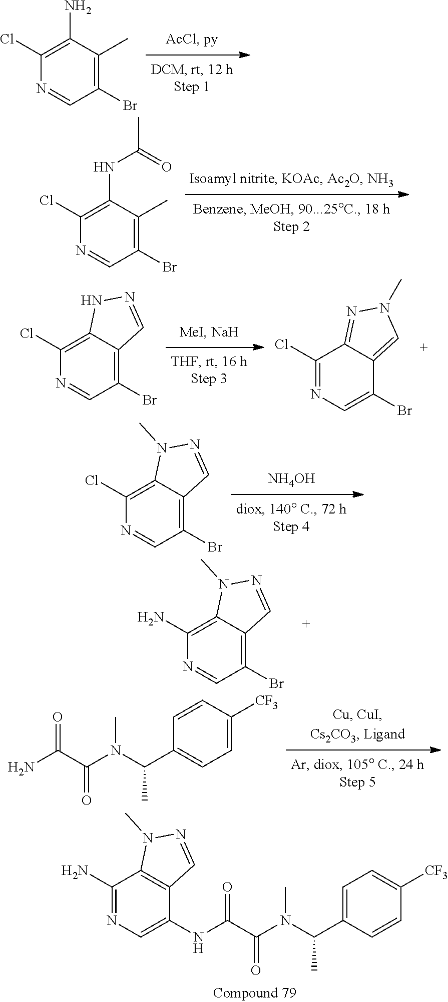

### Step 1: The Synthesis of N-(5-Bromo-2-chloro-4-methyl-3-pyridyl)acetamide

A solution of Acetyl chloride (7.44 g, 94.82 mmol, 5.75 mL) in dichloromethane (30 mL) was added dropwise to a stirred slurry of 5-bromo-2-chloro-4-methyl-pyridin-3-amine (20 g, 90.30 mmol) and Pyridine (7.50 g, 94.82 mmol, 7.64 mL) in dichloromethane (300 mL) at 0° C. The resulting mixture was allowed to warm to 25° C. and stirred for 12 hr. The resulting mixture (clear solution) was diluted with water (200 mL) and dichloromethane (500 mL, product started to precipitate from the solution after addition of water). The organic layer was separated, washed with water one more time (100 mL), dried over sodium sulfate and concentrated in vacuo to afford N-(5-bromo-2-chloro-4-methyl-3-pyridyl)acetamide (23 g, 87.28 mmol, 96.66% yield) as light-yellow solid.

LCMS (ESI): [M+H]⁺m/z: calcd 262.2; found 262.8; Rt=0.940 min.

### Step 2: The Synthesis of 4-Bromo-7-chloro-1H-pyrazolo[3,4-c]pyridine

Potassium Acetate (17.13 g, 174.56 mmol, 10.91 mL) and Acetic anhydride (26.73 g, 261.84 mmol, 24.71 mL) were added to a slurry of the N-(5-bromo-2-chloro-4-methyl-3-pyridyl)acetamide (23 g, 87.28 mmol) in dry benzene (776.90 mL). The reaction mixture was warmed to 70° C. and Isoamyl nitrite (20.45 g, 174.56 mmol, 23.37 mL) was added. The resulting mixture was stirred at 90° C. (oil bath temp.) for 16 hr.

The insoluble material was then filtered off while hot, and the filtrate was concentrated in vacuo to afford crude N-acylated product (34 g), it was dissolved in methanol (388.45 mL) and Ammonia, anhydrous (29.73 g, 1.75 mol, 34.57 mL) was vigorously bubbled through it for 1 hr. The resulting mixture was stirred at 25° C. for 1 hr, and then concentrated in vacuo. The residue was diluted with water and the precipitate was filtered, washed with water (4*25 mL), and the dissolved in ethyl acetate (700 mL). The small quantity of insoluble material was filtered off, the filtrate was dried over sodium sulfate and concentrated in vacuo to afford 4-bromo-7-chloro-1H-pyrazolo[3,4-c]pyridine (16 g, 68.83 mmol, 78.86% yield) as yellow solid.

LCMS (ESI): [M+H]⁺m/z: calcd 232.0; found 232.0; Rt=0.880 min.

### Step 3: The Synthesis of 4-Bromo-7-chloro-1-methyl-pyrazolo[3,4-c]pyridine and 4-Bromo-7-chloro-2-methyl-pyrazolo[3,4-c]pyridine

Sodium hydride (in oil dispersion) 60% dispersion in mineral oil (3.30 g, 82.59 mmol, 60% purity) was added portionwise to the solution of 4-bromo-7-chloro-1H-pyrazolo[3,4-c]pyridine (16 g, 68.83 mmol) in dry THF (295.68 mL). After 30 min reaction mixture was cooled to 0° C. and iodomethane (12.70 g, 89.48 mmol, 5.57 mL) was added in one portion and the rection mixture was allowed to warm to 25° C. and stirred for 16 hr. The reaction mixture was quenched with saturated aqueous ammonium chloride solution (30 mL) and concentrated in vacuo. The residue was diluted with water (100 mL) and extracted with dichloromethane (2*200 mL). The combined organic extracts were dried over sodium sulfate and concentrated in vacuo to afford crude mixture of isomeric products, which was separated by column chromatography on silica gel using chloroform/MTBE gradient (0-100% MTBE) to afford 4-bromo-7-chloro-1-methyl-pyrazolo[3,4-c]pyridine (4.5 g, 18.26 mmol, 26.52% yield) as light-yellow solid (1st eluting isomer); and 4-bromo-7-chloro-2-methyl-pyrazolo[3,4-c]pyridine (8.9 g, 36.11 mmol, 52.46% yield) as light-yellow solid (2nd eluting isomer).

### 1^{st} Eluting Isomer:

¹H NMR (400 MHz, CDCl₃) δ (ppm) 4.46 (s, 3H), 8.07 (s, 1H), 8.12 (s, 1H).

### 2^{nd} Eluting Isomer:

¹H NMR (400 MHz, CDCl₃) δ (ppm) 4.35 (s, 3H), 8.04 (s, 1H), 8.06 (s, 1H).

### Step 4: The Synthesis of 4-Bromo-1-methyl-pyrazolo[3,4-c]pyridin-7-amine

A mixture of 4-bromo-7-chloro-1-methyl-pyrazolo[3,4-c]pyridine (3 g, 12.17 mmol), Ammonia aqueous (63.00 g, 449.41 mmol, 70 mL, 25% purity) and 1.4-dioxane (40 mL) was stirred in autoclave at 140° C. (inner temp.) for 72 hr. The HNMR of the aliquot showed only traces of product. The reaction mixture was stirred at 140-155° C. for further 72 hr. The reaction mixture was concentrated to dryness and purified by column chromatography on silica gel using chloroform/acetonitrile gradient (0-100% acetonitrile) to afford 4-bromo-1-methyl-pyrazolo[3,4-c]pyridin-7-amine (1.6 g, 7.05 mmol, 57.90% yield) as pink solid.

LCMS (ESI): [M+H]⁺m/z: calcd 227.0; found 227.0; Rt=0.755 min.

### Step 5: The Synthesis of N-(7-Amino-1-methyl-pyrazolo[3,4-c]pyridin-4-yl)-N′-methyl-N′—[(1S)-1-[4-(trifluoromethyl)phenyl]ethyl]oxamide (Compound 79)

A mixture of N′-methyl-N′—[(1S)-1-[4-(trifluoromethyl)phenyl]ethyl]oxamide (140.77 mg, 513.30 μmol), 4-bromo-1-methyl-pyrazolo[3,4-c]pyridin-7-amine (100 mg, 440.41 μmol), copper (5 mg, 78.68 μmol), Copper (I) iodide (70.00 mg, 367.55 μmol, 12.46 μL), cesium carbonate (200.89 mg, 616.57 μmol) and (1R,2R)—N1,N2-dimethylcyclohexane-1,2-diamine (70.00 mg, 492.13 μmol) in 1,4-dioxane (6 mL) was stirred in a sealed vial under argon at 105° C. for 24 hr. The reaction mixture was cooled down and submitted to reverse phase HPLC (column: XBridge BEH C18 100×19 mm, 5 μm; mobile phase: 40-40-65% 0-1-5 min H₂O/MeOH/0.1% NH₄OH; flow rate: 30 mL/min (loading pump 4 mL/min methanol)) to afford Compound 79-N-(7-amino-1-methyl-pyrazolo[3,4-c]pyridin-4-yl)-N′-methyl-N′—[(1S)-1-[4-(trifluoromethyl)phenyl]ethyl]oxamide (55 mg, 130.83 μmol, 29.71% yield) as light-yellow solid.

¹H NMR (600 MHz, DMSO-d₆) δ (ppm) 1.56-1.70 (m, 3H), 2.57-2.81 (m, 3H), 4.22-4.32 (m, 3H), 5.15-5.83 (m, 1H), 6.09-6.59 (m, 2H), 7.53-7.60 (m, 1H), 7.63-7.67 (m, 1H), 7.72-7.78 (m, 2H), 7.79-7.96 (m, 2H), 10.50-10.92 (m, 1H).

LCMS (ESI): [M+H]⁺m/z: calcd 421.2; found 421.4; Rt=2.718 min.

### Example 167. Synthesis of N-(7-Amino-1-methyl-pyrazolo[3,4-c]pyridin-4-yl)-N′-methyl-N′—[(1R)-1-[4-(trifluoromethyl)phenyl]ethyl]oxamide (Compound 80)

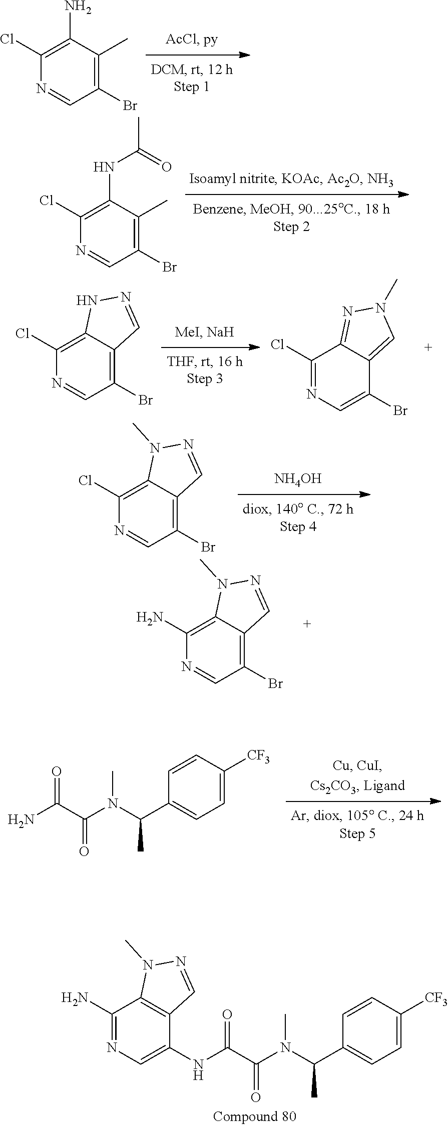

### The synthesis of 4-bromo-1-methyl-pyrazolo[3,4-c]pyridin-7-amine (Steps 1-4) was described for Compound 79

### Step 5: The Synthesis of N-(7-Amino-1-methyl-pyrazolo[3,4-c]pyridin-4-yl)-N′-methyl-N′—[(1R)-1-[4-(trifluoromethyl)phenyl]ethyl]oxamide (Compound 80)

A mixture of N′-methyl-N′—[(1R)-1-[4-(trifluoromethyl)phenyl]ethyl]oxamide (140.77 mg, 513.30 μmol), 4-bromo-1-methyl-pyrazolo[3,4-c]pyridin-7-amine (100 mg, 440.41 μmol), copper (5 mg, 78.68 μmol), Copper (I) iodide (70.00 mg, 367.55 μmol, 12.46 μL), cesium carbonate (200.89 mg, 616.57 μmol) and (1R,2R)—N1,N2-dimethylcyclohexane-1,2-diamine (70.00 mg, 492.13 μmol) in 1,4-dioxane (6 mL) was stirred in a sealed vial under argon at 105° C. for 24 hr. The reaction mixture was cooled down and submitted to reverse phase HPLC (column: XBridge BEH C18 100×19 mm, 5 μm; mobile phase: 40-40-65% 0-1-5 min H₂O/MeOH/0.1% NH₄OH; flow rate: 30 mL/min (loading pump 4 mL/min methanol)) to afford crude product (64 mg, 70% purity by LCMS), which was re-purified by reverse phase HPLC (column: XSelectCSH PFP 100×19 mm, 5 μm; mobile phase: Oct. 10, 1965% 0-1-5 min H₂O/ACN, flow rate: 30 mL/min (loading pump 4 mL/min acetonitrile)) to afford Compound 80-N-(7-amino-1-methyl-pyrazolo[3,4-c]pyridin-4-yl)-N′-methyl-N′—[(1R)-1-[4-(trifluoromethyl)phenyl]ethyl]oxamide (23 mg, 54.71 μmol, 12.42% yield) as light-yellow solid.

¹H NMR (600 MHz, DMSO-d₆) δ (ppm) 1.55-1.69 (m, 3H), 2.58-2.81 (m, 3H), 4.22-4.28 (m, 3H), 5.15-5.82 (m, 1H), 6.22 (s, 2H), 7.53-7.60 (m, 1H), 7.62-7.68 (m, 1H), 7.73-7.78 (m, 2H), 7.79-7.97 (m, 2H), 10.52-10.84 (m, 1H).

LCMS (ESI): [M+H]⁺m/z: calcd 421.2; found 421.2; Rt=2.742 min.

### Example 168. Synthesis of N-(7-Amino-1-methyl-pyrazolo[3,4-c]pyridin-4-yl)-N′-methyl-N′—[(1S)-1-[4-(1,1,2,2,2-pentafluoroethyl)phenyl]ethyl]oxamide (Compound 83) and N-(7-Amino-1-methyl-pyrazolo[3,4-c]pyridin-4-yl)-N′-methyl-N′—[(1R)-1-[4-(1,1,2,2,2-pentafluoroethyl)phenyl]ethyl]oxamide (Compound 81)

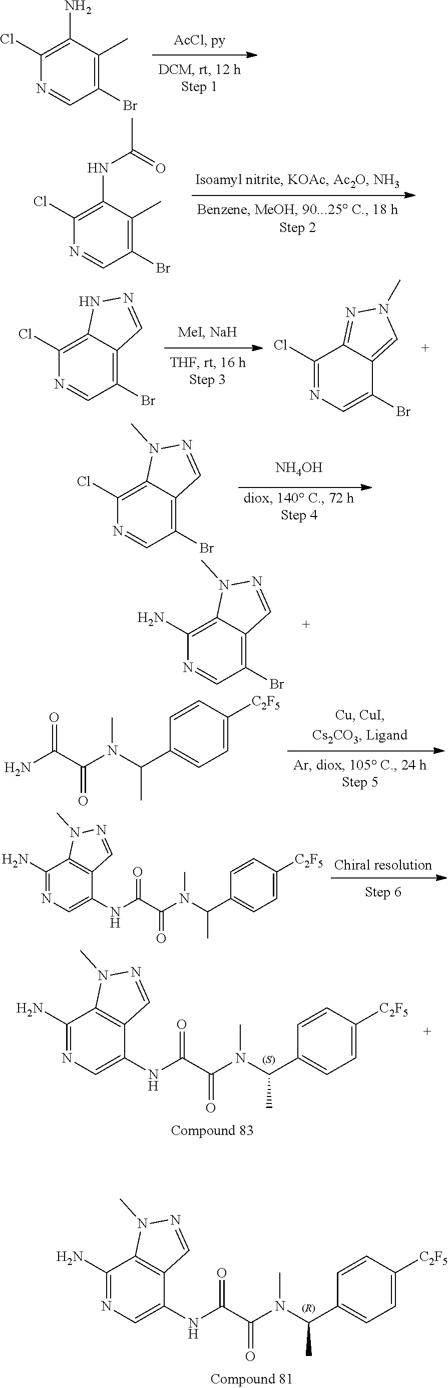

### The synthesis of 4-bromo-1-methyl-pyrazolo[3,4-c]pyridin-7-amine (Steps 1-4) was described for Compound 79

### The synthesis description for N′-methyl-N′-[1-[4-(1,1,2,2,2-pentafluoroethyl)phenyl]ethyl]oxamide is described for Compound 123, Compound 121, Compound 131

### Step 5: The Synthesis of N-(7-Amino-1-methyl-pyrazolo[3,4-c]pyridin-4-yl)-N′-methyl-N′-[1-[4-(1,1,2,2,2-pentafluoroethyl)phenyl]ethyl]oxamide

A mixture of N′-methyl-N′-[1-[4-(1,1,2,2,2-pentafluoroethyl)phenyl]ethyl]oxamide (240 mg, 740.18 μmol), 4-bromo-1-methyl-pyrazolo[3,4-c]pyridin-7-amine (150 mg, 660.62 μmol), copper (5 mg, 78.68 μmol), Copper (I) iodide (105.00 mg, 551.33 μmol, 18.68 μL), cesium carbonate (301.34 mg, 924.86 μmol) and rac-(1R,2R)—N1,N2-dimethylcyclohexane-1,2-diamine (105.00 mg, 738.19 μmol) in 1,4-dioxane (7 mL) was stirred in a sealed vial under argon at 105° C. for 24 hr. The resulting mixture was cooled down and submitted to reverse phase HPLC (column: XBridge BEH C18 100×19 mm, 5 μm; mobile phase: 40-40-65% 0-1-5 min H₂O/MeOH/0.1% NH₄OH, flow rate: 30 mL/min (loading pump 4 mL/min methanol)) to afford crude product (200 mg 54% purity by LCMS), which was re-purified by reverse phase HPLC (column: XSelectCSH PFP 100×19 mm, 5 μm; mobile phase: 20-20-65% 0-1-5 min H₂O/ACN, flow rate: 30 mL/min (loading pump 4 mL/min acetonitrile)) to afford N-(7-amino-1-methyl-pyrazolo[3,4-c]pyridin-4-yl)-N′-methyl-N′-[1-[4-(1,1,2,2,2-pentafluoroethyl)phenyl]ethyl]oxamide (43 mg, 91.41 μmol, 13.84% yield) as yellow solid.

LCMS (ESI): [M+H]⁺m/z: calcd 471.0; found 471.0; Rt=3.042 min.

### Step 6: The Synthesis of N-(7-Amino-1-methyl-pyrazolo[3,4-c]pyridin-4-yl)-N′-methyl-N′—[(1S)-1-[4-(1,1,2,2,2-pentafluoroethyl)phenyl]ethyl]oxamide (Compound 83) and N-(7-Amino-1-methyl-pyrazolo[3,4-c]pyridin-4-yl)-N′-methyl-N′—[(1R)-1-[4-(1,1,2,2,2-pentafluoroethyl)phenyl]ethyl]oxamide (Compound 81)

Racemic N-(7-amino-1-methyl-pyrazolo[3,4-c]pyridin-4-yl)-N′-methyl-N′-[1-[4-(1,1,2,2,2-pentafluoroethyl)phenyl]ethyl]oxamide (43 mg, 91.41 μmol) was submitted to preparative chiral HPLC (Column: Chiralpak AD-H—VI (250*21 mm, 5mkm); Mobile phase: Hexane-IPA-MeOH, 50-25-25 Flow Rate: 12 mL/min) to afford Compound 83-N-(7-amino-1-methyl-pyrazolo[3,4-c]pyridin-4-yl)-N′-methyl-N′—[(1S)-1-[4-(1,1,2,2,2-pentafluoroethyl)phenyl]ethyl]oxamide (20 mg, 42.52 μmol, 46.51% yield)(RetTime=21.744 min.) as beige solid; and Compound 81-N-(7-amino-1-methyl-pyrazolo[3,4-c]pyridin-4-yl)-N′-methyl-N′—[(1R)-1-[4-(1,1,2,2,2-pentafluoroethyl)phenyl]ethyl]oxamide (21 mg, 44.64 μmol, 48.84% yield)(RetTime=49.563 min.) as beige solid.

### Compound 83:

Analytical RT (Chiralpak AD-H (250×4.6 mm, 5 mkm)-8; Hexane: IPA: MeOH, 50:25:25; Flow Rate: 0.6 mL/min)=22.345 min.

¹H NMR (600 MHz, dmso) δ 1.56-1.70 (m, 3H), 2.59-2.86 (m, 3H), 4.23-4.29 (m, 3H), 5.14-5.89 (m, 1H), 6.21 (s, 2H), 7.61 (d, 1H), 7.67 (d, 1H), 7.72 (d, 2H), 7.79-7.95 (m, 2H), 10.63-10.80 (m, 1H).

LCMS (ESI): [M+H]⁺m/z: calcd 471.2; found 471.2; Rt=2.665 min.

### Compound 81:

Analytical RT (Chiralpak AD-H (250×4.6 mm, 5 mkm)-8; Hexane: IPA: MeOH, 50:25:25; Flow Rate: 0.6 mL/min)=48.902 min.

¹H NMR (600 MHz, dmso) δ 1.55-1.73 (m, 3H), 2.58-2.86 (m, 3H), 4.18-4.28 (m, 3H), 5.14-5.82 (m, 1H), 6.21 (s, 2H), 7.61 (d, 1H), 7.67 (d, 1H), 7.70-7.75 (m, 2H), 7.80-7.94 (m, 2H), 10.56-10.82 (m, 1H).

LCMS (ESI): [M+H]⁺m/z: calcd 471.2; found 471.2; Rt=2.673 min.

### Example 169. Synthesis of N-(7-Amino-1-methyl-pyrazolo[3,4-c]pyridin-4-yl)-N′-methyl-N′—[(1R)-1-[2-fluoro-4-(trifluoromethyl)phenyl]ethyl]oxamide (Compound 84) and N-(7-Amino-1-methyl-pyrazolo[3,4-c]pyridin-4-yl)-N′-methyl-N′—[(1S)-1-[2-fluoro-4-(trifluoromethyl)phenyl]ethyl]oxamide (Compound 82)

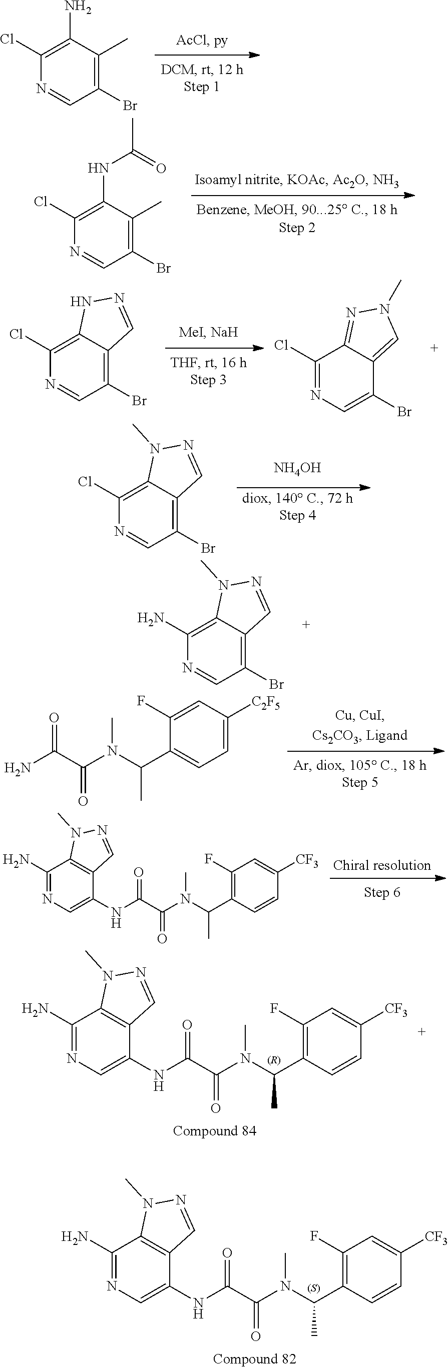

### The synthesis of 4-bromo-1-methyl-pyrazolo[3,4-c]pyridin-7-amine (Steps 1-4) was described for Compound 79

### The synthesis description for N′-[1-[2-fluoro-4-(trifluoromethyl)phenyl]ethyl]-N′-methyl-oxamide is described for Compound 54 and Compound 74

### Step 5: The Synthesis of N-(7-Amino-1-methyl-pyrazolo[3,4-c]pyridin-4-yl)-N′-[1-[2-fluoro-4-(trifluoromethyl)phenyl]ethyl]-N′-methyl-oxamide

A mixture of N′-[1-[2-fluoro-4-(trifluoromethyl)phenyl]ethyl]-N′-methyl-oxamide (300 mg, 1.03 mmol), 4-bromo-1-methyl-pyrazolo[3,4-c]pyridin-7-amine (200 mg, 880.82 μmol), copper (5 mg, 78.68 μmol), Copper (I) iodide (140 mg, 735.10 μmol, 24.91 μL), cesium carbonate (401.78 mg, 1.23 mmol) and rel-(1R,2R)—N1,N2-dimethylcyclohexane-1,2-diamine (140 mg, 984.25 μmol) in 1,4-dioxane (7 mL) was stirred in a sealed vial under argon at 105° C. for 18 hr. The resulting mixture was cooled down and submitted to reverse phase HPLC (column: XBridge BEH C18 100×19 mm, 5 μm; mobile phase: 40-40-65% 0-1-5 min H₂O/MeOH/0.1% NH₄OH, flow rate: 30 mL/min (loading pump 4 mL/min methanol)) to afford crude product (200 mg 88% purity by LCMS), which was re-purified by reverse phase HPLC (column: XSelectCSH PFP 100×19 mm, 5 μm; mobile phase: Oct. 10, 1965% 0-1-5 min H₂O/ACN, flow rate: 30 mL/min (loading pump 4 mL/min acetonitrile)) to afford N-(7-amino-1-methyl-pyrazolo[3,4-c]pyridin-4-yl)-N′-[1-[2-fluoro-4-(trifluoromethyl)phenyl]ethyl]-N′-methyl-oxamide (144 mg, 328.48 μmol, 37.29% yield) as beige solid.

LCMS (ESI): [M+H]⁺m/z: calcd 439.2; found 439.2; Rt=2.742 min.

### Step 6: The Synthesis of N-(7-Amino-1-methyl-pyrazolo[3,4-c]pyridin-4-yl)-N′-methyl-N′—[(1R)-1-[2-fluoro-4-(trifluoromethyl)phenyl]ethyl]oxamide (Compound 84) and N-(7-Amino-1-methyl-pyrazolo[3,4-c]pyridin-4-yl)-N′-methyl-N′—[(1S)-1-[2-fluoro-4-(trifluoromethyl)phenyl]ethyl]oxamide (Compound 82)

Racemic N-(7-amino-1-methyl-pyrazolo[3,4-c]pyridin-4-yl)-N′-[1-[2-fluoro-4-(trifluoromethyl)phenyl]ethyl]-N′-methyl-oxamide (144 mg, 328.48 μmol) was submitted to preparative chiral HPLC (Column: CHIRALPAK IC (250×21 mm, 5 mkm)-II; Mobile Phase: Hexane (0.1% DEA): IPA: MeOH, 50:25:25 Flow Rate: 12 mL/min) to afford Compound 84-N-(7-amino-1-methyl-pyrazolo[3,4-c]pyridin-4-yl)-N′-methyl-N′—[(1R)-1-[2-fluoro-4-(trifluoromethyl)phenyl]ethyl]oxamide (52 mg, 118.62 μmol, 36.11% yield)(RetTime=14.598 min) as beige solid; and Compound 82-N-(7-amino-1-methyl-pyrazolo[3,4-c]pyridin-4-yl)-N′-methyl-N′—[(1S)-1-[2-fluoro-4-(trifluoromethyl)phenyl]ethyl]oxamide (55 mg, 125.46 μmol, 38.19% yield)(RetTime=20.480 min) as beige solid.

### Compound 84:

Analytical RT (Chiralpak IC (250×4.6 mm, 5 mkm)-2; Hexane (0.1% EDA): IPA: MeOH, 50:25:2; Flow Rate: 0.6 mL/min)=14.491 min.

¹H NMR (600 MHz, dmso) δ 1.49-1.71 (m, 3H), 2.67-2.94 (m, 3H), 4.25 (s, 3H), 5.53-5.92 (m, 1H), 6.21 (s, 2H), 7.61-7.65 (m, 1H), 7.67-7.77 (m, 2H), 7.77-7.84 (m, 1H), 7.88-7.92 (m, 1H), 10.50-10.79 (m, 1H).

LCMS (ESI): [M+H]⁺m/z: calcd 439.2; found 439.2; Rt=2.366 min.

### Compound 82:

Analytical RT (Chiralpak IC (250×4.6 mm, 5 mkm)-2; Hexane (0.1% EDA): IPA: MeOH, 50:25:2; Flow Rate: 0.6 mL/min)=19.339 min.

¹H NMR (600 MHz, dmso) 8 1.52-1.71 (m, 3H), 2.66-2.92 (m, 3H), 4.25 (s, 3H), 5.49-5.89 (m, 1H), 6.21 (s, 2H), 7.60-7.65 (m, 1H), 7.68-7.77 (m, 2H), 7.77-7.84 (m, 1H), 7.87-7.93 (m, 1H), 10.53-10.69 (m, 1H).

LCMS (ESI): [M+H]⁺m/z: calcd 439.2; found 439.2; Rt=2.275 min.

The absolute stereochemistry was determined independently

### Example 170. Synthesis of N-(4-amino-1H-pyrazolo[4,3-c]pyridin-7-yl)-N′-methyl-N′-[1-[5-(trifluoromethyl)-2-pyridyl]ethyl]oxamide (Compound 178)

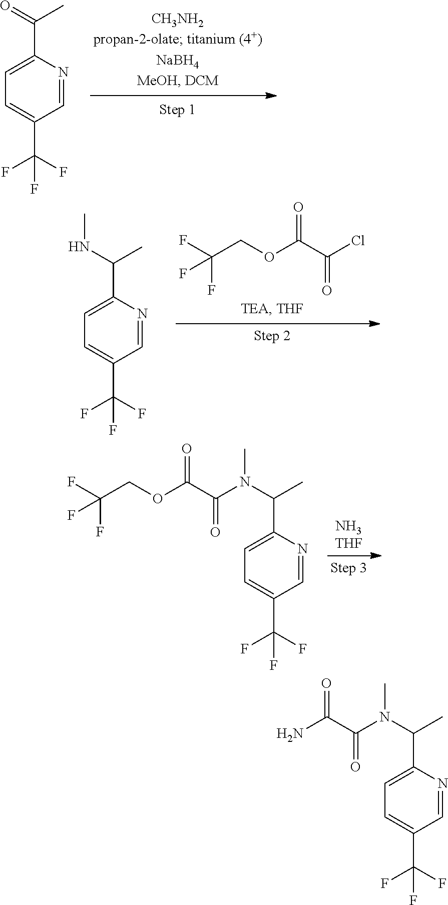

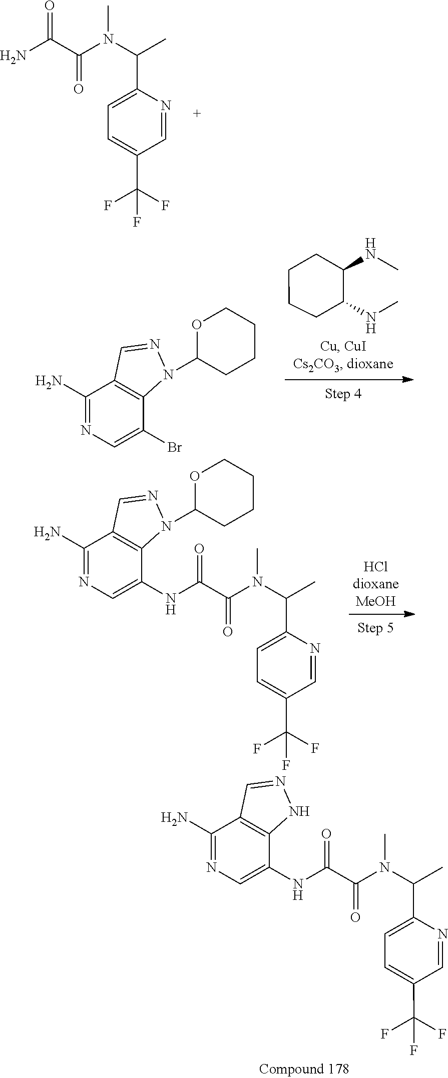

### Step 1: N-methyl-1-[5-(trifluoromethyl)-2-pyridyl]ethanamine

Propan-2-olate; titanium (4+)(180.33 mg, 634.47 μmol, 188.82 μL) was added in one portion to a solution of 1-[5-(trifluoromethyl)-2-pyridyl]ethanone (100.00 mg, 528.72 μmol), methanamine (428.40 mg, 1.38 mmol, 476.53 μL) in DCM (5 mL) and the resulting mixture was stirred for 12 hr at 25° C. After 12 hr Methanol (1 mL) was added to the RM, stirred for 10 min and Sodium Borohydride (14.00 mg, 370.11 μmol, 13.04 μL) was added and stirred for 30 min. The reaction mixture was quenched with K₂CO₃ (5 mL, sat. aq.) and extracted with DCM (2×10 mL). The combined organic layer was washed with water (5 mL), dried over anhydrous sodium sulfate and evaporated in vacuo to give N-methyl-1-[5-(trifluoromethyl)-2-pyridyl]ethanamine (90 mg, 440.76 μmol, 83.36% yield) as a yellow oil.

LCMS (ESI): [M+H]⁺m/z: calcd 205.1; found 205.0; Rt=0.553 min.

### Step 2:2,2,2-trifluoroethyl 2-oxo-2-[1-[5-(trifluoromethyl)-2-pyridy]ethylamino]acetate

2,2,2-trifluoroethyl 2-chloro-2-oxo-acetate (100.76 mg, 528.91 μmol) was added dropwise to a stirred solution of N-methyl-1-[5-(trifluoromethyl)-2-pyridyl]ethanamine (90 mg, 440.76 μmol) and TEA (156.10 mg, 1.54 mmol, 215.02 μL) in THF (9.94 mL) at 0° C., stirred for 1 hr at 0° C. The reaction mixture was used in the next step.

LCMS (ESI): [M+H]⁺m/z: calcd 359.09; found 359.0; Rt=1.314 min.

### Step 3: N′-[1-[5-(trifluoromethyl)-2-pyridyl]ethyl]oxamide

Ammonia (148.43 mg, 8.72 mmol) was bubbled trough a reaction mixture at 20° C., stirred for 1 hr at 20° C. and 8 hr at 25° C. Reaction mixture was filtered, solid washed with THF (2×5 mL), filtrate concentrated in vacuo to give crude N′-[1-[5-(trifluoromethyl)-2-pyridyl]ethyl]oxamide (65 mg, 248.85 μmol, 57.10% yield). Crude product was submitted to HPLC (column: XBridge BEH C18 5 μm 130A; Oct. 10, 2045% 0-1-5 min H₂O/ACN, flow rate: 30 mL/min) to give pure N′-[1-[5-(trifluoromethyl)-2-pyridyl]ethyl]oxamide (65 mg, 248.85 μmol, 57.10% yield) as a yellow gum.

LCMS (ESI): [M+H]⁺m/z: calcd 276.11; found 276.0; Rt=2.160 min.

### Step 4: N-(4-amino-1-tetrahydropyran-2-yl-pyrazolo[4,3-c]pyridin-7-yl)-N′-methyl-N′-[1-[5-(trifluoromethyl)-2-pyridyl]ethyl]oxamide

Copper (790.73 μg, 12.44 μmol), Copper (I) iodide (23.70 mg, 124.43 μmol, 4.22 μL), cesium carbonate (121.62 mg, 373.28 μmol) was added to a stirred solution of N′-[1-[5-(trifluoromethyl)-2-pyridyl]ethyl]oxamide (0.065 g, 248.85 μmol), 7-bromo-1-tetrahydropyran-2-yl-pyrazolo[4,3-c]pyridin-4-amine (77.64 mg, 261.29 μmol), rac-(1R,2R)—N1,N2-dimethylcyclohexane-1,2-diamine (17.70 mg, 124.43 μmol) in 1,4-dioxane (7.01 mL) under Ar atmosphere and stirred at 110° C. for 48 hr in closed vial. RM was filtered, solid washed with DCM (3 mL), combined filtrate was used in the next step.

LCMS (ESI): [M+H]⁺m/z: calcd 492.2; found 492.0; Rt=1.147 min.

### Step 5: The synthesis of N-(4-amino-1H-pyrazolo[4,3-c]pyridin-7-yl)-N′-methyl-N′-[1-[5-(trifluoromethyl)-2-pyridyl]ethyl]oxamide

Hydrogen chloride solution 4.0M in dioxane (44.51 mg, 1.22 mmol, 55.64 μL) was added to a stirred solution of N-(4-amino-1-tetrahydropyran-2-yl-pyrazolo[4,3-c]pyridin-7-yl)-N′-methyl-N′-[1-[5-(trifluoromethyl)-2-pyridyl]ethyl]oxamide (0.12 g, 244.17 μmol) in Methanol (5 mL) and stirred at 20° C. for 1 hr. Volatiles was evaporated in vacuo, the residue was submitted to HPLC (column: Chromatorex 18 SMB100-5T 100×19 mm 5 μm; 15-30% 0-5 min H₂O/ACN/0.1% NH₄OH, flow rate: 30 mL/min) to give racemic N-(4-amino-1H-pyrazolo[4,3-c]pyridin-7-yl)-N′-methyl-N′-[1-[5-(trifluoromethyl)-2-pyridyl]ethyl]oxamide (8 mg, 19.64 μmol, 8.04% yield) as a beige solid.

Enantiomers was not separated on analytical chiral HPLC.

¹H NMR (600 MHz, DMSO-d₆) § 1.59-1.68 (m, 3H), 2.72-3.03 (m, 3H), 5.68-5.81 (m, 1H), 6.66 (s, 2H), 7.56-7.74 (m, 2H), 8.17 (s, 1H), 8.22-8.29 (m, 1H), 8.92-9.02 (m, 1H), 10.33-10.58 (m, 1H), 12.58-12.84 (m, 1H).

LCMS (ESI): [M+H]⁺m/z: calcd 408.15; found 408.2; Rt=1.903 min.

### Example 171. Cellular Assay-SDMA in—Cell Western Protocol

A HAP1 MTAP-isogenic cell line pair was acquired from Horizon Discovery (HZGHC004894c005) and maintained in DMEM (ThermoFisher 11965)+10% FBS (Gemini 100-500) in a humidified, 10% CO₂ tissue culture incubator. The SAM-cooperative PRMT5 inhibitor, GSK3326595, was sourced from SelleckChem and maintained as a 10 mM DMSO stock. All test compounds are maintained as 10 mM DMSO stocks.

On Day 0, MTAP-intact or MTAP-deleted cells are seeded in a 384-well plate, and incubated in a humidified, 5% CO₂ tissue culture incubator for 16-24 hours. On Day 1, the test compounds are dispensed to wells at defined concentrations using a Tecan D300e digital dispenser (n=4), and the volume of DMSO is normalized to highest class volume. Each plate includes wells dosed with defined concentrations of GSK33226595 as a plate control. The compounds are incubated with cells for 24 hours in a humidified, 5% CO₂ tissue culture incubator.

On Day 2, the compound-treated cells are fixed with a final concentration of 4% formaldehyde. The cells are then washed/permeabilized with 1×PBS+0.1% Triton X-100, and then blocked with 5% goat serum/1X TBS. The fixed cells are then incubated overnight at 4° C. with a primary SDMA antibody cocktail (Cell Signaling 13222).

On Day 3, the cells are washed with 1×PBS+0.1% Triton X-100, and then incubated at room temperature for 1 hour with a NIR fluorescent secondary antibody cocktail that also contains DRAQ5 (LiCor 926-32211 and VWR 10761-508). The cells are washed with 1×PBS+0.1% Triton X-100, and then washed again with ddH₂O. The plates are then imaged using a NIR fluorescent imager (LiCor Odyssey).

For data analysis, the SDMA signal is normalized to the DRAQ5 signal. Assay background is determined by the signal from wells treated with 1 μM GSK3326595, and subtracted from every well. The data are plotted as % of the DMSO control wells for the MTAP-intact and the MTAP-deleted cell lines independently, and fitted to the 4-parameter logistic (4-PL) Hill equation with maximal effect constrained to 0. The fit was performed using GraphPad Prism or the default IC₅₀ fitting procedure in Dotmatics Studies 5.4 as part of a customized data analysis protocol.

The data obtained in this experiment is presented in Table 1, columns 4-6.

### Example 172. Viability Assay Protocol

A HAP1 MTAP-isogenic cell line pair was acquired from Horizon Discovery (HZGHC004894c005) and maintained in DMEM (ThermoFisher 11965)+10% FBS (Gemini 100-500) in a humidified, 5 or 10% CO₂ tissue culture incubator. All test compounds are maintained as 10 mM DMSO stocks.

On Day 0, MTAP-intact and MTAP-deleted cells are seeded in a 96-well plate, and incubated in a humidified, 5 or 10% CO₂ tissue culture incubator for 16-24 hours. On Day 1, the test compounds are dispensed to wells at defined concentrations using a Tecan D300e digital dispenser (n=3), and the volume of DMSO is normalized to highest class volume (0.2%). The compound-treated plates are incubated for 7 days in a humidified, 5 or 10% CO₂ tissue culture incubator.

On Day 7, the plates are removed from the tissue culture incubator and allowed to equilibrate to room temperature. Then either a 1/2 volume CellTiter-Glo Luminescent Cell Viability Assay reagent (Promega G7572) is added to each well, or the media is removed from every well and a 1:3 dilution of CellTiter-Glo 2.0 Cell Viability Assay reagent (Promega G9241) in 1×PBS is added. Ten minutes after addition, the luminescent signal is detected by an Envision plate reader. The data are plotted as % of the DMSO control wells for the MTAP-intact and the MTAP-deleted cell lines independently, and fitted to the 4-parameter logistic (4-PL) Hill equation with maximal effect constrained to 0. The fit was performed using GraphPad Prism or the default IC₅₀ fitting procedure in Dotmatics Studies 5.4 as part of a customized data analysis protocol.

The data obtained in this experiment is presented in Table 1, column 8.

### Example 173. Combination Viability Assay Protocol

A SW1573 MTAP-isogenic cell line pair can be generated by either reconstituting MTAP gene expression, or by introducing an empty control vector, in the MTAP-deleted SW1573 parental cell line. The cell lines can be maintained in DMEM+10% FBS in a humidified, 5% CO₂ tissue culture incubator. All test compounds can be maintained as 10 mM DMSO stocks.

On Day 0, MTAP-intact and MTAP-deleted cells can be seeded in a 384-well plate, and incubated in a humidified, 5% CO₂ tissue culture incubator for 16-24 hours. On Day 1, the test compounds can be dispensed to wells at defined concentrations (n=2), and the volume of DMSO can be normalized to highest class volume. The compound-treated plates can be incubated for 7 days in a humidified, 5% CO₂ tissue culture incubator.

On Day 7, the plates can be removed from the tissue culture incubator and allowed to equilibrate to room temperature. Relative viability can be assessed by addition of CellTiter-Glo reagent, and data can be plotted as % of DMSO control for each compound in each cell line, with a 4-parameter fit non-linear regression model (GraphPad Prism). Synergy can be determined according to the HSA model by the Combenefit software package (Version 2.021). PRMT5 inhibitors and MAT2A inhibitors represents a potential clinical combination in MTAP-deleted tumors

Marjon et al (Cell Reports 2016) and Kalev et al (Cancer Cell 2021) identify MAT2A as a therapeutic target in MTAP-deleted tumors. The combination of a MAT2A inhibitor with an inhibitor that selectively targets PRMT5 in MTAP-null cells can be assessed to determine whether this would present a rational therapeutic strategy. Combination of a MAT2A inhibitor (e.g., AG-270) with an exemplar MTAP^{null}-selective PRMT5 inhibitor in a 7-day viability assay in the MTAP-null SW1573 cancer cell line can demonstrate enhanced cellular viability defects. PRMT5 inhibitors and MAPK or KRASG12C inhibitors represent a potential clinical combination in MTAP-deleted, KRAS-mutated tumors

MTAP-deletion can co-occur with mutations in the KRAS gene (e.g., KRASG12C). Therapies targeting KRAS or other members of the MAPK pathway (e.g., MAPK3, MAPK1, MEK1 and MEK2) exist. The combination of these inhibitors with an inhibitor that selectively targets PRMT5 in MTAP-null cells can be assessed to determine whether this would present a therapeutic strategy.

Combination of a KRASG12C inhibitor (e.g., AMG-510), with an exemplar MTAP^{null}-selective PRMT5 inhibitor in a 7-day viability assay in the MTAP-null SW1573 cancer cell line can demonstrate enhanced cellular viability defects.

Combination of MAPK1/MAPK3 inhibitors (e.g., ulixertinib and SCH772984), with an exemplar MTAP^{null}-selective PRMT5 inhibitor in a 7-day viability assay in the MTAP-null SW1573 cancer cell line can demonstrate enhanced cellular viability defects.

Combination of MEK inhibitors (e.g., trametinib) with an exemplar MTAP^{null}-selective PRMT5 inhibitor in a 7-day viability assay in the MTAP-null SW1573 cancer cell line can demonstrate enhanced cellular viability defects.

What is claimed is:

1. A compound of Formula 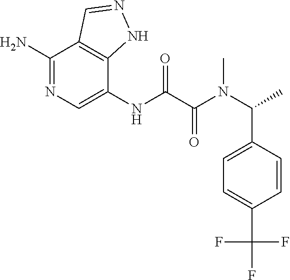 [SMILES: C[C@H](c1ccc(C(F)(F)F)cc1)N(C)C(=O)C(=O)Nc1cnc(N)c2cn[nH]c12]
or a pharmaceutically acceptable salt thereof.

2. A composition comprising the compound of claim 1, or a pharmaceutically acceptable salt thereof, and one or more pharmaceutically acceptable excipients.

3. A compound of Formula

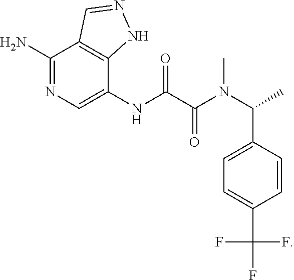 [SMILES: C[C@H](c1ccc(C(F)(F)F)cc1)N(C)C(=O)C(=O)Nc1cnc(N)c2cn[nH]c12]

4. A composition comprising the compound of claim 3, and one or more pharmaceutically acceptable excipients.
